(19) United States
(12) Patent Application Publication
(10) Pub. No.: US 2021/0317134 A1
(43) Pub. Date: Oct. 14, 2021

(54) UBIQUITIN-SPECIFIC-PROCESSING PROTEASE 7 (USP7) MODULATORS AND USES THEREOF

(71) Applicant: RAPT THERAPEUTICS, INC., South San Francisco, CA (US)

(72) Inventors: Jacob Bradley Schwarz, South San Francisco, CA (US); John Michael Ketcham, San Mateo, CA (US); Maureen Kay Reilly, San Francisco, CA (US); Berenger Biannic, San Mateo, CA (US); Minna Hue Thanh Bui, Oakland, CA (US); Dennis X. Hu, San Mateo, CA (US); David Juergen Wustrow, Los Gatos, CA (US); Emily Karbarz, Burlingame, CA (US); Xinping Han, Daly City, CA (US); Grant Shibuya, South San Francisco, CA (US); Jack Maung, Daly City, CA (US); Maksim Osipov, Redwood City, CA (US)

(21) Appl. No.: 16/810,376

(22) Filed: Mar. 5, 2020

Related U.S. Application Data
(60) Provisional application No. 62/815,177, filed on Mar. 7, 2019.

(51) Int. Cl. C07D 495/04; C07D 491/048; C07D 405/14; C07D 471/04; C07D 519/00; C07D 498/04

(52) U.S. Cl. CPC C07D 495/04; C07D 491/048; C07D 498/04; C07D 471/04; C07D 519/00; C07D 405/14

(57) ABSTRACT

Disclosed herein, inter alia, compounds and methods of use thereof for the modulation of USP7 activity.

### CROSS-REFERENCES TO RELATED APPLICATIONS

This application claims the benefit of U.S. Provisional application No. 62/815,177, filed Mar. 7, 2019, which is incorporated herein by reference in its entirety and for all purposes.

### BACKGROUND

Ubiquitination is an important form of post-translational modification that can determine protein's fate. While ubiquitin itself is a small and conserved protein, its covalent conjugation to protein substrates and to other ubiquitin molecules is a tightly controlled process involving complex cellular machinery. Perhaps, the most prominent and well-known function of ubiquitin is to target a protein for degradation by the 26S proteasome. This is done via isopeptide bond formation between the carboxy-terminal Gly on the ubiquitin and ε-amino group of lysine side chains of the protein substrate.

The ubiquitin-substrate system is further diversified via the process of polyubiquitination, during which a ubiquitin molecule's C-terminal Gly is conjugated with one of the seven Lys residues on another ubiquitin (Lys6, Lys11, Lys27, Lys29, Lys33, Lys48, or Lys63) or with the N-terminus to form linear chains. While Lys48-linked and Lys11-linked polyubiquitination has been shown mostly to target protein substrates for 26S-mediated degradation, other functions of ubiquitination continue to unfold. For example, Lys63-polyubiquitination is involved in DNA repair and endocytosis, while both Lys63-linked and linear polyubiquitination has been demonstrated to regulate immunity via NF-κB activation. Accordingly, the biological complexity of ubiquitination is complex and suggests that enzymes involved in this process must have remarkable specificity to correctly carry out their unique functions. E1, E2, and E3 are enzymes that together facilitate the multistep process of ubiquitin-substrate conjugation, and deubiquitinat-ing enzymes (deubiquitinases or DUBs) carry out the reverse steps of breaking the isopeptide bond.

There are approximately 100 DUBs known in the human genome, any of which could be playing a key role in the ubiquitin-proteasome system as well as other biological processes. The main functions for which DUBs are responsible include: (a) liberation of ubiquitin from protein substrates (e.g., to remove degradation signal), (b) editing of polyubiquitin signal on protein substrates to change the fate of the protein, (c) disassembling polyubiquitin chains to free up ubiquitin monomers, (d) cleaving ubiquitin precursors or adducts to regenerate active ubiquitin.

The DUBs are subdivided into five families: ubiquitin C-terminal hydrolases (UCHs), ubiquitin-specific proteases (USPs), ovarian tumor proteases (OTUs), Josephins and JAB 1/MPN/MOV34 metalloenzymes (JAMM/MPN+). The first four families (UCH, USP, OTU, and Josephin) are cysteine proteases, while JAMM/MPN+ are zinc metalloproteases. Among these, UCHs and USPs are the best characterized, and USPs represent more than half of the known human DUBs. The reaction mechanism of the cysteine protease DUB families is the same as that of the cysteine protease superfamily. Each enzyme active site requires the interplay between three conserved residues forming the catalytic triad: a cysteine, a histidine, and an aspartic acid. The initial attack creates a negatively charged transition state stabilized by the oxy-anion hole. The intermediate is a thiohemiacetal stabilized through interactions with the active site residues as an incoming water molecule liberates the lysine side chain from the conjugated ubiquitin or substrate. A nucleophilic attack of the water creates another negatively charged transition state, which rearranges to free up the carboxylate terminus of the N-terminal ubiquitin and restores the enzyme to its apo form.

Recently, increased biological understanding has led to numerous DUBs being implicated in various diseases spanning oncology, neurodegeneration, hematology, and infectious diseases. Most recently, Bingol et al. carried out elegant experiments in vitro and in vivo to illustrate the role of USP30 as an antagonist of Parkin-mediated mitophagy, suggesting the inhibition of USP30 as a potential therapy for Parkinson's disease (PD). The hunt for DUB antagonists is thus actively carried out by academic and pharmaceutical companies alike. This is illustrated through chemically diverse small molecules that have been reported to inhibit one or more of the UCH and USP family members.

Indeed, the covalent attachment of ubiquitin to proteins is an important step in the degradation of proteins via the 26S proteasome. See e.g., Metzger M B et. al. [J Cell Sci. 2012; 125:531-7] Deubiquitinating enzymes (DUBs), also known as deubiquitinating peptidases, deubiquitinating isopeptidases, deubiquitinases, ubiquitin proteases, ubiquitin hydrolases, ubiquitin isopeptidases, are a large group of proteases that cleave ubiquitin from proteins and other molecules. [Nijman S M, et al. Cell. 2005; 123:773-86] DUBs can prevent the degradation of proteins by cleaving the peptide or isopeptide bond between ubiquitin and its substrate protein. In humans there are nearly 100 DUB genes, which can be classified into two main classes: cysteine proteases and metalloproteases. The cysteine proteases comprise ubiquitin-specific proteases (USPs), ubiquitin C-terminal hydrolases (UCHs), Machado-Josephin domain proteases (MJDs) and ovarian tumour proteases (OTU). The metalloprotease group contains only the Jab1/Mov34/Mprl Padl N-terminal+(MPN+) (JAMM) domain proteases.

Ubiquitin-specific-processing protease 7 (USP7), also known as ubiquitin carboxyl-terminal hydrolase 7 or herpesvirus-associated ubiquitin-specific protease (HAUSP), is an enzyme that in humans is encoded by the USP7 gene. USP7 or HAUSP is a DUB enzyme that cleaves ubiquitin from its substrates. Since ubiquitylation (polyubiquitination) is most commonly associated with the stability and degradation of cellular proteins, USP7 activity generally stabilizes its substrate proteins. [Shi D et. al. Cancer Biology and Therapy 2010 10:8 737-747]

USP7 is most popularly known as a direct antagonist of Mdm2, the E3 ubiquitin ligase for the tumor suppressor protein, p53. Normally, p53 levels are kept low in part due to Mdm2-mediated ubiquitylation and degradation of p53. In response to oncogenic insults, USP7 can deubiquitinate p53 and protect p53 from Mdm2-mediated degradation, indicating that it may possess a tumor suppressor function for the immediate stabilization of p53 in response to stress.

In addition, USP7 also plays a key role in the immunoregulatory transcription factor protein FOXP3. By de-ubiquitylating and preserving FOXP3, USP7 increases T regulatory cell (Treg) mediated suppression of tumor-infiltrating T effector cells, the latter being associated with improved clinical outcome for many solid tumors. Thus, USP7 functions to limit immune cell-mediated antitumor defenses. The observation that the accumulation of FOXP3+ Treg cells at the tumor or in draining lymph nodes signals poor prognosis further highlights the significance of this recently described second oncogenic mechanism of USP7. Thus, inhibitors of USP7 can exert in vivo antitumor activity by: 1) directly inhibiting tumor cell proliferation via Hdm2 and other targets; and 2) suppressing T regulatory cells via FOXP3, thereby facilitating the antitumor function of T effector cells.

Another important role of USP7 function involves the oncogenic stabilization of p53. Oncogenes such as Myc and E1A are thought to activate p53 through a p19 alternative reading frame (p19ARF, also called ARF)-dependent pathway, although some evidence suggests ARF is not essential in this process. A possibility is that USP7 provides an alternative pathway for safeguarding the cell against oncogenic insults.

USP7 can deubiquitinate histone H2B and this activity is associated with gene silencing in Drosophila. USP7 associates with a metabolic enzyme, GMP synthetase (GMPS) and this association stimulates USP7 deubiquitinase activity towards H2B. The USP7-GMPS complex is recruited to the polycomb (Pc) region in Drosophila and contributes to epigenetic silecing of homeotic genes.

USP7 was originally identified as a protein associated with the ICP0 protein of herpes simplex virus (HSV), hence the alternate name Herpesvirus Associated USP (HAUSP). ICP0 is an E3-ubiquitin ligase that is involved in ubiquitination and subsequent degradation of itself and certain cellular proteins. USP7 has been shown to regulate the auto-ubiquitination and degradation of ICP0.

More recently, an interaction between USP7 and the EBNA1 protein of Epstein-Barr virus (EBV) (another herpes virus) was also discovered. This interaction is particularly interesting given the oncogenic potential (potential to cause cancer) of EBV, which is associated with several human cancers. EBNA1 can compete with p53 for binding USP7. Stabilization by USP7 is important for the tumor suppressor function of p53. In cells, EBNA1 can sequester USP7 from p53 and thus attenuate stabilization of p53, rendering the cells predisposed to turning cancerous. Compromising the function of p53 by sequestering USP7 is one way EBNA1 can contribute to the oncogenic potential of EBV. Additionally, human USP7 was also shown to form a complex with GMPS and this complex is recruited to EBV genome sequences. USP7 was shown to be important for histone H2B deubiquitination in human cells and for deubiquitination of histone H2B incorporated in the EBV genome. Thus, USP7 may also be important for regulation of viral gene expression. The fact that viral proteins have evolved so as to target USP7, underscores the significance of USP7 in tumor suppression and other cellular processes.

### BRIEF SUMMARY

Disclosed herein are compounds that modulate USP7 function and methods of using the same.

In an aspect is provided a compound of structural Formula (VII) or a pharmaceutically acceptable salt thereof:

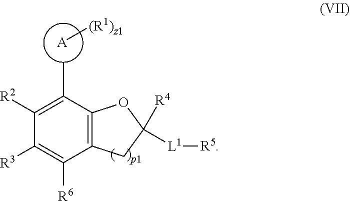

### Ring A is

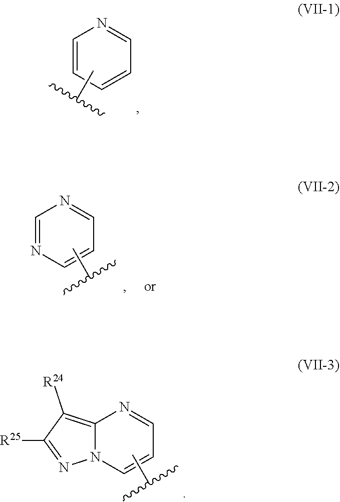

The symbol p1 is 1 or 2. L¹ is a bond, —O—, —S—, —NH—, —C(O)—, —C(O)NH, —C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R¹ is independently halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein two R¹ substituents bonded to adjacent carbon atoms may optionally be joined to form a substituted or unsubstituted cycloalkyl, substituted or unsubstituted heterocycloalkyl, substituted or unsubstituted aryl, or substituted or unsubstituted heteroaryl. R² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₂H, —SOᵥ₂NH₂, —NHNH₂, —ONH₂, —NHC(O)NHNH₂, —NHC(O)NH₂, —N(O)ₘ₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₃H, —SOᵥ₃NH₂, —NHNH₂, —ONH₂, —NHC(O)NHNH₂, —NHC(O)NH₂, —N(O)ₘ₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₄H, —SOᵥ₄NH₂, —NHNH₂, —ONH₂, —NHC(O)NHNH₂, —NHC(O)NH₂, —N(O)ₘ₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁵ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₅R^{5A}, —SOᵥ₅NR^{5B}R^{5C}, —NHNR^{5B}R^{5C}, —ONRS^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₆H, —SOᵥ₆NH₂, —NHNH₂, —ONH₂, —NHC(O)NHNH₂, —NHC(O)NH₂, —N(O)ₘ₆, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₄H, —SOᵥ24NH₂, —NHNH₂, —ONH₂, —NHC(O)NHNH₂, —NHC(O)NH₂, —N(O)ₘ₂₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁵ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₅R^{25A}, —SOᵥ₂₅NR^{25B}R^{25C}, —NHNR^{25B}R^{25C}, —ONR^{25B}R^{25C}, —NHC(O)NHNR^{25B}R^{25C}, —NHC(O)NR^{25B}R^{25C}, —N(O)ₘ₂₅, —NR^{25B}R^{25C}, —C(O)R^{25D}, —C(O)OR^{25D}, —C(O)NR^{25B}R^{25C}, —OR^{25A}, —NR^{25B}SO₂R^{25A}, —NR^{25B}C(O)R^{25D}, —NR^{25B}C(O)OR^{25D}, —NR^{25B}OR^{25D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R^{1A}, R^{1B}, R^{1C}, R^{1D}, R^{5A}, R^{5B}, R^{5C}, R^{5D}, R^{25A}, R^{25B}, R^{25C}, and R^{25D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C}, R^{5B} and R^{5C}, or R^{25B} and R^{25C} substituents bonded to the same nitrogen atom may optionally be joined to form a substituted or unsubstituted heterocycloalkyl or substituted or unsubstituted heteroaryl. The symbols n1, n2, n3, n4, n5, n6, n24, and n25 are independently an integer from 0 to 4. The symbols m1, m2, m3, m4, m5, m6, m24, m25, v1, v2, v3, v4, v5, v6, v24, and v25 are independently 1 or 2. The symbol z1 is an integer from 0 to 4.

In an aspect is provided a pharmaceutical composition, including a compound as described herein, including embodiments (e.g., structural Formulae (I), (II), (III), (IV), (V), (VI), (VII), (VII-1A), (VII-2A), (VII-3A), (VIII), (VIII-1), (VIII-2), (VIII-3), (VIII-4), (VIII-5), (VIII-6), (VIII-7), (VIII-8), (VIII-9), (VIII-10), (VIII-11), (VIII-12), (IX), (X), (X-A1), (XI) or (XII)) and a pharmaceutically acceptable excipient.

In another aspect is provided a method of inhibiting ubiquitin-specific-processing protease 7 (USP7), the method including contacting USP7 with a compound as described herein, including embodiments (e.g., structural Formulae (I), (II), (III), (IV), (V), (VI), (VII), (VII-1A), (VII-2A), (VII-3A), (VIII), (VIII-1), (VIII-2), (VIII-3), (VIII-4), (VIII-5), (VIII-6), (VIII-7), (VIII-8), (VIII-9), (VIII-10), (VIII-11), (VIII-12), (IX), (X), (X-A1), (XI) or (XII)), or a pharmaceutically acceptable salt thereof.

In an aspect, there is provided a method of treating or preventing a USP7-mediated disease or disorder, including administering to a patient in need thereof a therapeutically effective amount of a compound or pharmaceutical composition as described herein, including embodiments (e.g., structural Formulae (I), (II), (III), (IV), (V), (VI), (VII), (VII-1A), (VII-2A), (VII-3A), (VIII), (VIII-1), (VIII-2), (VIII-3), (VIII-4), (VIII-5), (VIII-6), (VIII-7), (VIII-8), (VIII-9), (VIII-10), (VIII-11), (VIII-12), (IX), (X), (X-A1), (XI) or (XII)), or a pharmaceutically acceptable salt thereof.

In another aspect, there is provided a method of treating a USP7-mediated cancer, including administering to a patient in need thereof a chemotherapeutic agent in combination with a therapeutically effective amount of a compound or pharmaceutical composition as described herein, including embodiments (e.g., structural Formulae (I), (II), (III), (IV), (V), (VI), (VII), (VII-1A), (VII-2A), (VII-3A), (VIII), (VIII-1), (VIII-2), (VIII-3), (VIII-4), (VIII-5), (VIII-6), (VIII-7), (VIII-8), (VIII-9), (VIII-10), (VIII-11), (VIII-12), (IX), (X), (X-A1), (XI) or (XII)), or a pharmaceutically acceptable salt thereof. In embodiments, the method further includes administering to the patient a chemotherapeutic agent in combination with a compound as described herein.

In another aspect, provided herein is a kit including a compound described herein (e.g., a USP7 inhibitor) or pharmaceutical compositions thereof. The kits are generally in the form of a physical structure housing various components, as described below, and may be utilized, for example, in practicing the methods described herein.

### DETAILED DESCRIPTION

Provided herein are, for example, compounds and compositions for inhibition of ubiquitin-specific-processing protease 7 (USP7), and pharmaceutical compositions comprising the same. Also provided herein are, for example, methods of treating or preventing a disease, disorder or condition, or a symptom thereof, mediated by modulation (e.g., inhibition) of USP7.

### I. DEFINITIONS

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3 dioxanyl, 1,3 dioxolanyl, 1,3 dithiolanyl, 1,3 dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1 dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydr-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen. Other non-limiting examples of heteroaryl include:

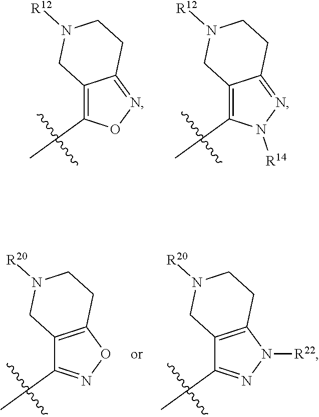

wherein R¹², R¹⁴, R²⁰ and R²² are as described herein, including embodiments.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker. In embodiments, the alkylarylene group has the formula;

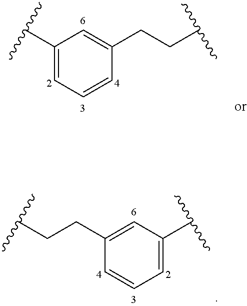 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₈ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (e.g., a floating substituent on a single ring), may be a substituent on any of the fused rings or spirocyclic rings (e.g., a floating substituent on multiple rings). When a substituent is attached to a ring, but not a specific atom (e.g., a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}-U-, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl, heteroalkyl, cycloalkyl, heterocycloalkyl, aryl, heteroaryl, substituted with at least one substituent selected from: oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds may exist in tautomeric forms, all such tautomeric forms of the compounds being within the scope hereof.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope hereof.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope hereof.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}R^{13B}, R^{13C}, R^{13D}, etc., wherein each of R^{13A}, R^{13B}, R^{13C}, R^{13D}, etc. is defined within the scope of the definition of R¹³ and optionally differently.

A “detectable moiety” as used herein refers to a moiety that can be covalently or noncovalently attached to a compound or biomolecule that can be detected for instance, using techniques known in the art. In embodiments, the detectable moiety is covalently attached. The detectable moiety may provide for imaging of the attached compound or biomolecule. The detectable moiety may indicate the contacting between two compounds. Exemplary detectable moieties are fluorophores, antibodies, reactive dies, radio-labeled moieties, magnetic contrast agents, and quantum dots. Exemplary fluorophores include fluorescein, rhodamine, GFP, coumarin, FITC, Alexa fluor, Cy3, Cy5, BODIPY, and cyanine dyes. Exemplary radionuclides include Fluorine-18, Gallium-68, and Copper-64. Exemplary magnetic contrast agents include gadolinium, iron oxide and iron platinum, and manganese.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents. In embodiments, compounds of the present disclosure contain both basic and acidic functionalities that allow the compounds to be converted into either base or acid addition salts. The neutral forms of the compounds may be regenerated by contacting the salt with a base or acid and isolating the parent compound in a conventional manner. The parent form of the compounds differs from the various salt forms in certain physical properties, such as solubility in polar solvents, but, unless specifically indicated, the salts disclosed herein are equivalent to the parent form of the compound for the purposes of the present disclosure.

In addition to salt forms, the present disclosure provides compounds which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of a compound to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

A “USP7 inhibitor” refers to a compound (e.g., a compound described herein) that reduces the function or activity of USP7 when compared to a control, such as, for example, the absence of the compound or a compound with known inactivity. As used herein, the terms “USP7 inhibitor” and “USP7 antagonist” and all other related art-accepted terms, many of which are set forth herein, refer to a compound capable of reducing (e.g., reducing relative to the absence of the inhibitor), either directly or indirectly, the activity and/or function USP7 receptor in an in vitro assay, an in vivo model, and/or other means indicative of therapeutic efficacy. The terms also refer to a compound that exhibits at least some therapeutic benefit in a human subject.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. In embodiments, the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences, with or without N-terminus methionine residues; immunologically tagged proteins; and the like.

A polypeptide, or a cell is “recombinant” when it is artificial or engineered, or derived from or contains an artificial or engineered protein or nucleic acid (e.g. non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway (e.g., USP7, p53, or Foxp3 pathway).

As defined herein, the term “activation”, “activate”, “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein (e.g., decreased in a disease).

The terms “agonist,” “activator,” “upregulator,” etc. refer to a substance capable of detectably increasing the expression or activity of a given gene or protein elative to the activity or function of the protein in the absence of the agonist. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist. In embodiments, an agonist is a molecule that interacts with a target to cause or promote an increase in the activation of the target. In embodiments, activators are molecules that increase, activate, facilitate, enhance activation, sensitize, or up-regulate, e.g., a gene, protein, ligand, receptor, or cell.

As defined herein, the term “inhibition,” “inhibit,” “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g., an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist. An antagonist prevents, reduces, inhibits, or neutralizes the activity of an agonist, and an antagonist can also prevent, inhibit, or reduce constitutive activity of a target, e.g., a target receptor, even where there is no identified agonist. In embodiments, inhibitors are molecules that decrease, block, prevent, delay activation, inactivate, desensitize, or down-regulate, e.g., a gene, protein, ligand, receptor, or cell. An inhibitor may also be defined as a molecule that reduces, blocks, or inactivates a constitutive activity. An “antagonist” is a molecule that opposes the action(s) of an agonist.

The terms “ubiquitin-specific-processing protease 7” or “USP7” or the like refer, in the usual and customary sense, to a protein (including homologs, isoforms, and functional fragments thereof) which can cleave ubiquitin from a substrate, e.g., a ubiquitinated protein. Exemplary substrates include FOXO4, p53/TP53, MDM2, ERCC6, DNMT1, UHRF1, PTEN, and DAXX, as known in the art. USP7 catalyzes the thiol-dependent hydrolysis of ester, thioester, amide, peptide and isopeptide bonds formed by the C-terminal Gly of ubiquitin (a 76-residue protein attached to proteins as an intracellular targeting signal). USP7 can be referred to by a number of different names in the scientific literature, including “uniquitin specific peptidase 7,” “ubiquitin-specific processing protease 7,” “deubiquitinating enzyme 7,” “unibuitin thioesterase 7,” “HAUSP,” “Herpes Virus-associated uniquitin specific protease,” “ubiquitincarboxyl terminal hydroxylase 7,” “EC 3.4.19.12,” “EC 3.1.2.15” or “TEF1.” The term includes any recombinant or naturally-occurring form of USP7 variants thereof that maintain USP7 activity (e.g. within at least 30%, 40%, 50%, 60%, 70%, 80%, 90%, 95%, or 100% activity compared to wildtype USP7). The term includes any mutant form of USP7 variants (e.g., frameshift mutations) thereof that maintain USP& activity (e.g. within at least 30%, 40%, 50%, 60%, 70%, 80%, 90%, 95%, or 100% activity compared to wildtype USP7). In embodiments, the USP7 protein encoded by the USP7 gene has the amino acid sequence set forth in or corresponding to Entrez 7874, UniProt Q93009, or NCBI Accession NP:_001308787.1, NP01273386.1, NP003461.2, NP001273387.1, or EAW85194.1. In embodiments, the amino acid sequence or nucleic acid sequence is the sequence known at the time of filing of the present application.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease.

As used herein, the term “cancer” refers to all types of cancer, neoplasm or malignant tumors found in mammals (e.g. humans), including leukemia, lymphoma, carcinoma and sarcoma. Exemplary cancers that may be treated with a compound or method provided herein include brain cancer, glioma, glioblastoma, neuroblastoma, prostate cancer, colorectal cancer, pancreatic cancer, cervical cancer, gastric cancer, ovarian cancer, lung cancer, cancer of the head, Hodgkin's Disease, and non-Hodgin's Lymphoma. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum. In some further instances, “cancer” refers to human cancers and carcinomas, sarcomas, adenocarcinomas, lymphomas, leukemias, etc., including solid and lymphoid cancers like kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including MDS, AML, ALL, ATLL and CML), or multiple myeloma.

As used herein, the term “inflammatory disease” refers to a disease or condition characterized by aberrant inflammation (e.g. an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis. Such conditions are frequently inextricably intertwined with other diseases, disorders and conditions. A non-limiting list of inflammatory-related diseases, disorders and conditions which may, for example, be caused by inflammatory cytokines, include, arthritis, kidney failure, lupus, asthma, psoriasis, colitis, pancreatitis, allergies, fibrosis, surgical complications (e.g., where inflammatory cytokines prevent healing), anemia, and fibromyalgia. Other diseases and disorders, which may be associated with chronic inflammation include Alzheimer's disease, congestive heart failure, stroke, aortic valve stenosis, arteriosclerosis, osteoporosis, Parkinson's disease, infections, inflammatory bowel disease (IBD), allergic contact dermatitis and other eczemas, systemic sclerosis, transplantation and multiple sclerosis. Some of the aforementioned diseases, disorders and conditions for which a compound (e.g., USP7 inhibitor) of the present disclosure may be particularly efficacious (due to, for example, limitations of current therapies) are described in more detail hereafter.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

The terms “treating” or “treatment” refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of a compound described herein. The administering step may consist of a single administration or may include a series of administrations. The length of the treatment period depends on a variety of factors, such as the severity of the condition, the age of the patient, the concentration of the compound, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “prevent” refers to a decrease in the occurrence of disease symptoms in a patient. As indicated above, the prevention may be complete (no detectable symptoms) or partial, such that fewer symptoms are observed than would likely occur absent treatment. In embodiments, prevent refers to slowing the progression of the disease, disorder or condition or inhibiting progression thereof to a harmful or otherwise undesired state.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). The therapeutically effective amount can be ascertained by measuring relevant physiological effects, and it can be adjusted in connection with the dosing regimen and diagnostic analysis of the subject's condition, and the like. By way of example, measurement of the serum level of a USP7 inhibitor (or, e.g., a metabolite thereof) at a particular time post-administration may be indicative of whether a therapeutically effective amount has been administered.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan. Adjusting the dose to achieve maximal therapeutic window efficacy or toxicity in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages, which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal) compatible with the preparation. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, administration includes contact (e.g., in vitro or ex vivo) of a compound to the cell, as well as contact of a compound to a fluid, where the fluid is in contact with the cell.

“Co-administer” means that a composition described herein is administered at the same time, just prior to, or just after the administration of one or more additional therapies. The compounds of the disclosure can be administered alone or can be coadministered to the patient. Co-administration is meant to include simultaneous or sequential administration of the compounds individually or in combination (more than one compound). The compositions of the present disclosure can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “modulator” refers to a composition that increases or decreases the level of a target molecule or the function of a target molecule or the physical state of the target of the molecule elative to the activity or function of the protein in the absence of the modulator. In some embodiments, a USP7 associated disease modulator is a compound that reduces the severity of one or more symptoms of a disease associated with USP7 (e.g. cancer, inflammatory disease, autoimmune disease, or infectious disease). A USP7 modulator is a compound that increases or decreases the activity or function or level of activity or level of function of USP7. A modulator may act alone, or it may use a cofactor, e.g., a protein, metal ion, or small molecule. Examples of modulators include small molecule compounds and other bioorganic molecules. Numerous libraries of small molecule compounds (e.g., combinatorial libraries) are commercially available and can serve as a starting point for identifying a modulator. The skilled artisan is able to develop one or more assays (e.g., biochemical or cell-based assays) in which such compound libraries can be screened in order to identify one or more compounds having the desired properties; thereafter, the skilled medicinal chemist is able to optimize such one or more compounds by, for example, synthesizing and evaluating analogs and derivatives thereof. Synthetic and/or molecular modeling studies can also be utilized in the identification of an activator

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule. In embodiments, the terms “modulate,” “modulation” and the like refer to the ability of a molecule (e.g., an activator or an inhibitor) to increase or decrease the function or activity of USP7, either directly or indirectly, relative to the absence of the molecule.

The term “associated” or “associated with” in the context of a substance or substance activity or function associated with a disease (e.g., a protein associated disease, a cancer associated with USP7 activity, USP7 associated cancer, USP7 associated disease (e.g., cancer, inflammatory disease, autoimmune disease, or infectious disease)) means that the disease (e.g., cancer, inflammatory disease, autoimmune disease, or infectious disease) is caused by (in whole or in part), or a symptom of the disease is caused by (in whole or in part) the substance or substance activity or function. For example, a cancer associated with USP7 activity or function may be a cancer that results (entirely or partially) from aberrant USP7 function (e.g. enzyme activity, protein-protein interaction, signaling pathway) or a cancer wherein a particular symptom of the disease is caused (entirely or partially) by aberrant USP7 activity or function. As used herein, what is described as being associated with a disease, if a causative agent, could be a target for treatment of the disease. For example, a cancer associated with USP7 activity or function or a USP7 associated disease (e.g., cancer, inflammatory disease, autoimmune disease, or infectious disease), may be treated with a compound described herein (e.g., USP7 modulator or USP7 inhibitor), in the instance where increased USP7 activity or function (e.g. signaling pathway activity) causes the disease (e.g., cancer, inflammatory disease, autoimmune disease, or infectious disease). For example, an inflammatory disease associated with USP7 activity or function or an USP7 associated inflammatory disease, may be treated with an USP7 modulator or USP7 inhibitor, in the instance where increased USP7 activity or function (e.g. signaling pathway activity) causes the disease.

The term “mediated” in the context of a substance or substance activity or function causing a disease (e.g., a cancer mediated by USP7 activity, USP7-mediated cancer, USP7 mediated disease (e.g., cancer, inflammatory disease, autoimmune disease, or infectious disease)) means that the disease (e.g., cancer, inflammatory disease, autoimmune disease, or infectious disease) is caused by (in whole or in part), or a symptom of the disease is caused by (in whole or in part) the substance or substance activity or function. For example, a cancer mediated by USP7 activity or function may be a cancer that results (entirely or partially) from aberrant USP7 function (e.g. enzyme activity, protein-protein interaction, signaling pathway) or a cancer wherein a particular symptom of the disease is caused (entirely or partially) by aberrant USP7 activity or function. As used herein, what is described as being a disease mediated by a substance or substance activity, if a causative agent, could be a target for treatment of the disease. For example, a cancer mediated by USP7 activity or function or a USP7 mediated disease (e.g., cancer, inflammatory disease, autoimmune disease, or infectious disease), may be treated with a compound described herein (e.g., USP7 modulator or USP7 inhibitor), in the instance where increased USP7 activity or function (e.g. signaling pathway activity) causes the disease (e.g., cancer, inflammatory disease, autoimmune disease, or infectious disease). For example, an inflammatory disease mediated by USP7 activity or function may be treated with an USP7 modulator or USP7 inhibitor, in the instance where increased USP7 activity or function (e.g. signaling pathway activity) causes the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propogated to other signaling pathway components. For example, binding of a USP7 with a compound as described herein may reduce the level of a product of the USP7 catalyzed reaction or the level of a downstream derivative of the product or binding may reduce the interactions between the USP7 or a reaction product and downstream effectors or signaling pathway components (e.g., USP7, p53, or Foxp3 pathways), resulting in changes in cell growth, proliferation, or survival.

The phrase “in a sufficient amount to affect a change” means that there is a detectable difference between a level of an indicator measured before (e.g., a baseline level) and after administration of a particular therapy. Indicators include any objective parameter (e.g., serum concentration) or subjective parameter (e.g., a subject's feeling of well-being).

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like. The term “proliferative activity” encompasses an activity that promotes, that is necessary for, or that is specifically associated with, for example, normal cell division, as well as cancer, tumors, dysplasia, cell transformation, metastasis, and angiogenesis.

“Substantially pure” indicates that a component makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition (percentage in a weight per weight basis).

The terms “specifically binds” and “selectively binds,” when referring to a ligand/receptor, antibody/antigen, or other binding pair, indicates a binding reaction which is determinative of the presence of the protein in a heterogeneous population of proteins and other biologics. Thus, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant or mutein thereof, with an affinity that is at least two-fold greater, at least 10-times greater, at least 20-times greater, or at least 100-times greater than the affinity with any other antibody, or binding composition derived therefrom. In embodiments, the antibody will have an affinity that is greater than about 10⁹ liters/mol, as determined by, e.g., Scatchard analysis (Munsen, et al. (1980) Analyt. Biochem. 107:220-239).

The terms “nucleic acid,” “nucleic acid molecule,” “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, DNA, RNA, messenger RNA (mRNA), complementary DNA (cDNA), recombinant polynucleotides, vectors, probes, primers and the like.

As used herein, the terms “variants” and “homologs” are used interchangeably to refer to amino acid or nucleic acid sequences that are similar to reference amino acid or nucleic acid sequences, respectively. The term encompasses naturally-occurring variants and non-naturally-occurring variants. Naturally-occurring variants include homologs (polypeptides and nucleic acids that differ in amino acid or nucleotide sequence, respectively, from one species to another), and allelic variants (polypeptides and nucleic acids that differ in amino acid or nucleotide sequence, respectively, from one individual to another within a species). Thus, variants and homologs encompass naturally occurring amino acid and nucleic acid sequences encoded thereby and their isoforms, as well as splice variants of a protein or gene. The terms also encompass nucleic acid sequences that vary in one or more bases from a naturally-occurring nucleic acid sequence but still translate into an amino acid sequence that corresponds to the naturally-occurring protein due to degeneracy of the genetic code. Non-naturally-occurring variants and homologs include polypeptides and nucleic acids that comprise a change in amino acid or nucleotide sequence, respectively, where the change in sequence is artificially introduced (e.g., muteins); for example, the change is generated in the laboratory by human intervention (“hand of man”). Therefore, non-naturally occurring variants and homologs may also refer to those that differ from the naturally-occurring sequences by one or more conservative substitutions and/or tags and/or conjugates.

The term “muteins” as used herein refers broadly to mutated recombinant proteins. These proteins usually carry single or multiple amino acid substitutions and are frequently derived from cloned genes that have been subjected to site-directed or random mutagenesis, or from completely synthetic genes. In embodiments, a mutein is a protein with one amino acid substitution relative to the known amino acid sequence. In embodiments, a mutein is a protein with one amino acid substitution relative to the known amino acid sequence.

### II. COMPOUNDS

In an aspect is provided a compound of structural Formula (VII) or a pharmaceutically acceptable salt thereof:

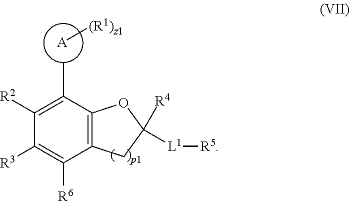

### Ring A is

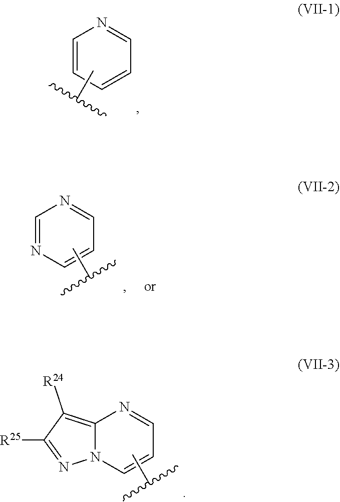

The symbol p is 1 or 2. L¹ is a bond, —O—, —S—, —NH—, —C(O)—, —C(O)NH, —C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R¹ is independently halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein two R¹ substituents bonded to adjacent carbon atoms may optionally be joined to form a substituted or unsubstituted cycloalkyl, substituted or unsubstituted heterocycloalkyl, substituted or unsubstituted aryl, or substituted or unsubstituted heteroaryl. R² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₂H, —SOᵥ₂NH₂, —NHNH₂, —ONH₂, —NHC(O)NHNH₂, —NHC(O)NH₂, —N(O)ₘ₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₃H, —SOᵥ₃NH₂, —NHNH₂, —ONH₂, —NHC(O)NHNH₂, —NHC(O)NH₂, —N(O)ₘ₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₄H, —SOᵥ₄NH₂, —NHNH₂, —ONH₂, —NHC(O)NHNH₂, —NHC(O)NH₂, —N(O)ₘ₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁵ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₆H, —SOᵥ₆NH₂, —NHNH₂, —ONH₂, —NHC(O)NHNH₂, —NHC(O)NH₂, —N(O)ₘ₆, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₄H, —SOᵥ₂₄NH₂, —NHNH₂, —ONH₂, —NHC(O)NHNH₂, —NHC(O)NH₂, —N(O)ₘ₂₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁵ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₅R^{25A}, —SOᵥ₂₅NR^{25B}R^{25C}, —NHNR^{25B}R^{25C}, —ONR^{25B}R^{25C}, —NHC(O)NHNR^{25B}R^{25C}, —NHC(O)NR^{25B}R^{25C}, —N(O)ₘ₂₅, —NR^{25B}R^{25C}, —C(O)R^{25D}, —C(O)OR^{25D}, —C(O)NR^{25B}R^{25C}, —OR^{25A}, —NR^{25B}SO₂R^{25A}, —NR^{25B}C(O)R^{25D}, —NR^{25B}C(O)OR^{25D}, —NR^{25B}OR^{25D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R^{1A}, R^{1B}, R^{1C}, R^{1D}, R^{5A}, R^{5B}, R^{5C}, R^{5D}, R^{25A}, R^{25B}, R^{25C}, and R^{25D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C}, R^{5B} and R^{5C}, or R^{25B} and R^{25C} substituents bonded to the same nitrogen atom may optionally be joined to form a substituted or unsubstituted heterocycloalkyl or substituted or unsubstituted heteroaryl. The symbols n1, n2, n3, n4, n5, n6, n24, and n25 are independently an integer from 0 to 4. The symbols m1, m2, m3, m4, m5, m6, m24, m25, v1, v2, v3, v4, v5, v6, v24, and v25 are independently 1 or 2. The symbol z1 is an integer from 0 to 4.

In embodiments, the compound has structural Formula (IX):

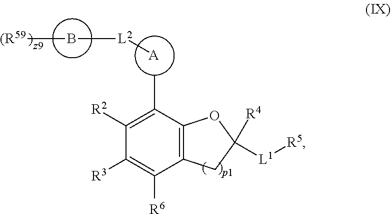

wherein ring A, R², R³, R⁴, R⁵, R⁶, L¹, and p1 are as described herein, including embodiments.

Ring B is substituted or unsubstituted cycloalkyl, substituted or unsubstituted heterocycloalkyl, substituted or unsubstituted aryl, or substituted or unsubstituted heteroaryl. It is understood when z9 is equal to or greater than 1, Ring B is substituted (e.g., independently substituted with a R⁵⁹ substituent). When z9 is 0, it is understood Ring B is unsubstituted. L² is a bond, —O—, —S—, —NH—, —C(O)—, —C(O)NH, —C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R⁵⁹ is independently halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —N₃, —SOₙ₅₉H, —SOᵥ₅₉NH₂, —NHNH₂, —ONH₂, —NHC(O)NHNH₂, —NHC(O)NH₂, —N(O)ₘ₅₉, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein two R⁵⁹ substituents bonded to adjacent atoms may optionally be joined to form a substituted or unsubstituted cycloalkyl, substituted or unsubstituted heterocycloalkyl, substituted or unsubstituted aryl, or substituted or unsubstituted heteroaryl. The symbol n59 is an integer from 0 to 4. The symbols m59 and v59 are independently 1 or 2. The symbol z9 is an integer from 0 to 6.

In embodiments, the compound has structural Formula (X):

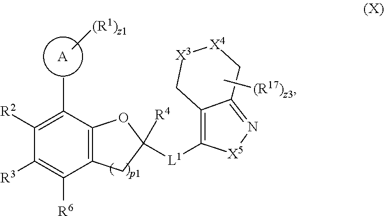

wherein ring A, R¹, R², R³, R⁴, R⁶, L¹, and p1 are as described herein, including embodiments.

### X³ is

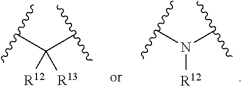

### X⁴ is

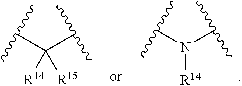

### X⁵ is —O— or

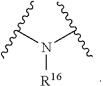

R¹² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —N₃, —CN, —SO₂CH₃, —SOₙ₁₂H, —SOᵥ₁₂NH₂, —NHNH₂, —ONH₂, —NHC(O)NHNH₂, —NHC(O)NH₂, —N(O)ₘ₁₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹³ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₃H, —SOᵥ₁₃NH₂, —NHNH₂, —ONH₂, —NHC(O)NHNH₂, —NHC(O)NH₂, —N(O)ₘ₁₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹² and R¹³ are optionally together oxo. R¹⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₄H, —SOᵥ₁₄NH₂, —NHNH₂, —ONH₂, —NHC(O)NHNH₂, —NHC(O)NH₂, —N(O)ₘ₁₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹⁵ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₁₅H, —SOᵥ₁₅NH₂, —NHNH₂, —ONH₂, —NHC(O)NHNH₂, —NHC(O)NH₂, —N(O)ₘ₁₅, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹⁴ and R¹⁵ are optionally together oxo. R¹⁶ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₆H, —SOᵥ₁₆NH₂, —NHNH₂, —ONH₂, —NHC(O)NHNH₂, —NHC(O)NH₂, —N(O)ₘ₁₆, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹⁷ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₁₇H, —SOᵥ₁₇NH₂, —NHNH₂, —ONH₂, —NHC(O)NHNH₂, —NHC(O)NH₂, —N(O)ₘ₁₇, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbols n12, n13, n14, n15, n16 and n17 are independently an integer from 0 to 4. The symbols m12, m13, m14, m15, m16, m17, v12, v13, v14, v15, v16 and v17 are independently 1 or 2. The symbol z3 is an integer from 0 to 4.

In embodiments, the compound has structural Formula (XI):

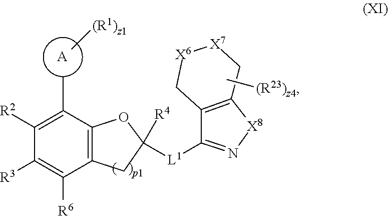

wherein ring A, R¹, R², R³, R⁴, R⁶, L¹, and p1 are as described herein, including embodiments.

### X⁶ is

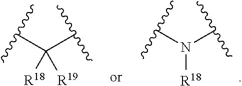

### X⁷ is

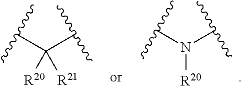

### X⁸ is —O— or

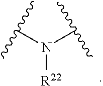

R¹⁸ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₈H, —SOᵥ₁₈NH₂, —NHNH₂, —ONH₂, —NHC(O)NHNH₂, —NHC(O)NH₂, —N(O)ₘ₁₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹⁹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₉H, —SOᵥ₁₉NH₂, —NHNH₂, —ONH₂, —NHC(O)NHNH₂, —NHC(O)NH₂, —N(O)ₘ₁₉, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹⁸ and R¹⁹ are optionally together oxo. R²⁰ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₂₀H, —SOᵥ₂₀NH₂, —NHNH₂, —ONH₂, —NHC(O)NHNH₂, —NHC(O)NH₂, —N(O)ₘ₂₀, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²¹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₂₁H, —SOᵥ₂₁NH₂, —NHNH₂, —ONH₂, —NHC(O)NHNH₂, —NHC(O)NH₂, —N(O)ₘ₂₁, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁰ and R²¹ are optionally together oxo. R²² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₂H, —SOᵥ₂₂NH₂, —NHNH₂, —ONH₂, —NHC(O)NHNH₂, —NHC(O)NH₂, —N(O)ₘ₂₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²³ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₂₃H, —SOᵥ₂₃NH₂, —NHNH₂, —ONH₂, —NHC(O)NHNH₂, —NHC(O)NH₂, —N(O)ₘ₂₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbols n18, n19, n20, n21, n22 and n23 are independently an integer from 0 to 4. The symbols m18, m19, m20, m21, m22, m23, v18, v19, v20, v21, v22 and v23 are independently 1 or 2. The symbol z4 is an integer from 0 to 4.

In embodiments, the compound has structural Formula (X-1A):

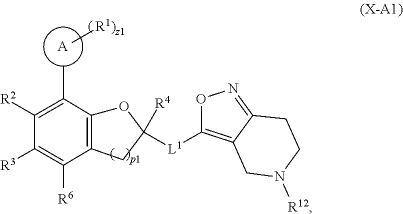

wherein ring A, R¹, z1, Ring A, R², R³, R⁴, R⁶, p1, L¹, and R¹² are as described herein, including embodiments.

In embodiments, the compound has structural Formula (XII):

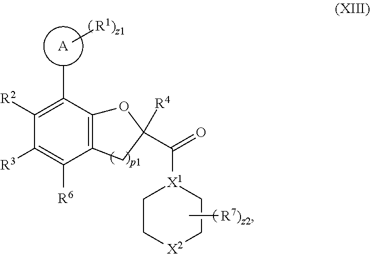

wherein ring A, R¹, R², R³, R⁴, R⁶, and p1 are as described herein, including embodiments.

### X¹ is

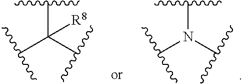

### X² is

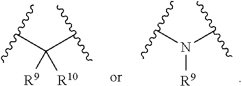

R⁷ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₇H, —SOᵥ₇NH₂, —NHNH₂, —ONH₂, —NHC(O)NHNH₂, —NHC(O)NH₂, —N(O)ₘ₇, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁸ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₈H, —SOᵥ₈NH₂, —NHNH₂, —ONH₂, —NHC(O)NHNH₂, —NHC(O)NH₂, —N(O)ₘ₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₉R^{9A}, —SOᵥ₉NR^{9B}R^{9C}, —NHNR^{9B}R^{9C}, —ONR^{9B}R^{9C}, —NHC(O)NHNR^{9B}R^{9C}, —NHC(O)NR^{9B}R^{9C}, —N(O)ₘ₉, —NR^{9B}R^{9C}, —C(O)R^{9D}, —C(O)OR^{9D}, —C(O)NR^{9B}R^{9C}, —OR^{9A}, —NR^{9B}SO₂R^{9A}, —NR^{9B}C(O)R^{9D}, —NR^{9B}C(O)OR^{9D}, —NR^{9B}OR^{9D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹⁰ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₁₀R^{10A}, —SOᵥ₁₀NR^{10B}R^{10C}, —NHNR^{10B}R^{10C}, —ONR^{10B}R^{10C}, —NHC(O)NHNR^{10B}R^{10C}, —NHC(O)NR^{10B}R^{10C}, —N(O)ₘ₁₀, —NR^{10B}R^{1C}, —C(O)R^{10D}, —C(O)OR^{10D}, —C(O)NR^{1B}R^{1C}, —OR^{10A}, —NR^{10B}SO₂R^{10A}, —NR^{10B}C(O)R^{10D}, —NR^{10B}C(O)OR^{10D}, —NR^{10B}OR^{10D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁹ and R¹⁰ are optionally together oxo. R⁹ and R¹⁰ substituents may optionally be joined to form a substituted or unsubstituted cycloalkyl, substituted or unsubstituted heterocycloalkyl, substituted or unsubstituted aryl, or substituted or unsubstituted heteroaryl. R^{9A}, R^{9B}, R^{9C}, R^{9D}, R^{10A}, R^{10B}, R^{10C} and R^{10D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{9B} and R^{9C} or R^{10B} and R^{10C} substituents bonded to the same nitrogen atom may optionally be joined to form a substituted or unsubstituted heterocycloalkyl or substituted or unsubstituted heteroaryl. The symbols n7, n8, n9 and n10 are independently an integer from 0 to 4. The symbols m7, m8, m9, m10, v7, v8, v9 and v10 are independently 1 or 2. The symbol z2 is an integer from 0 to 8.

In embodiments, the compound has structural Formula (XII):

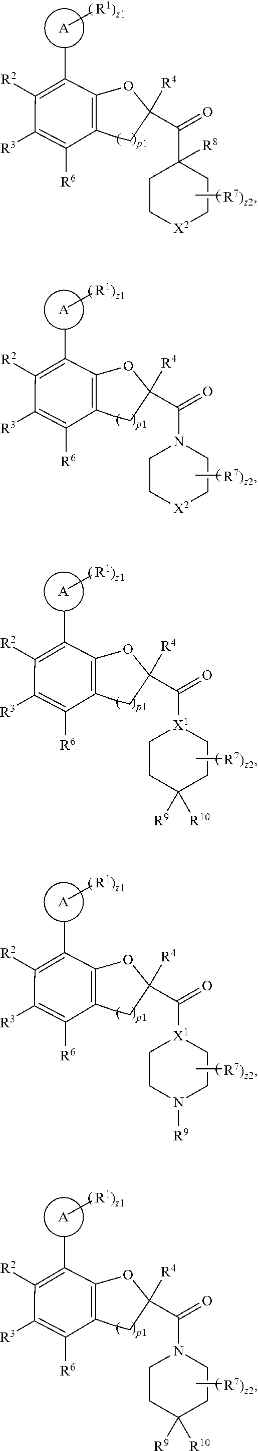

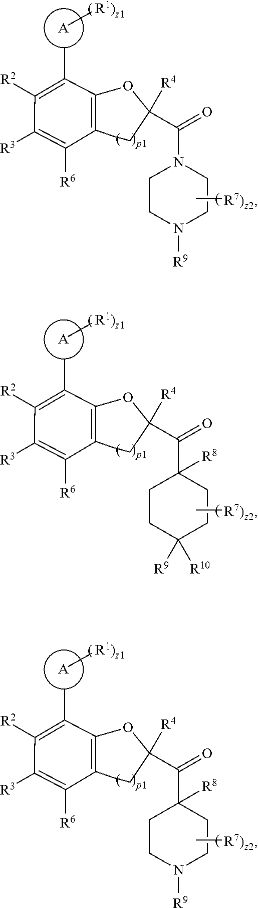

wherein R¹, z1, Ring A, R², R³, R⁶, p1, R⁴, R⁷, R⁸, X¹, X², z2, R⁹, and R¹⁰ are as described herein, including embodiments.

In embodiments, the compound has the structural Formula (I):

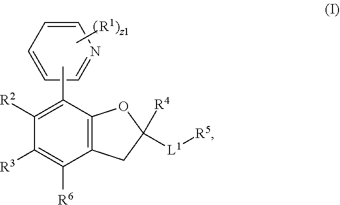

wherein R¹, z1, R², R³, R⁴, R⁶, L¹, and R⁵ are as described herein, including embodiments.

In embodiments, the compound has structural Formula (II):

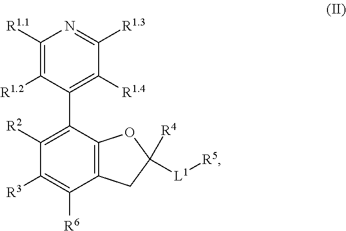

wherein R², R³, R⁴, R⁶, L¹, and R⁵ are as described herein, including embodiments. R^{1.1}, R^{1.2}, R^{1.3}, and R^{1.4} are each hydrogen or R¹ at a fixed position on the attached ring. R^{1.1}, R^{1.2}, R^{1.3}, and R^{1.4} may be hydrogen or any R¹ moiety described herein, including in any aspect, embodiment, example, figure, or claim.

R^{1.1} is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹², R¹³, and R¹⁴ are independently hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁H, —SOᵥ₁NH₂, —NHNH₂, —ONH₂, —NHC(O)NHNH₂, —NHC(O)NH₂, —N(O)ₘ₁, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{1.1} and R^{1.2} or R^{1.3} and R^{1.4} may optionally be joined to form a substituted or unsubstituted cycloalkyl, substituted or unsubstituted heterocycloalkyl, substituted or unsubstituted aryl, or substituted or unsubstituted heteroaryl. In embodiments, at least one of R^{1.1}, R^{1.2}, R^{1.3} and R^{1.4} is not hydrogen.

In embodiments, the compound has structural Formula (III):

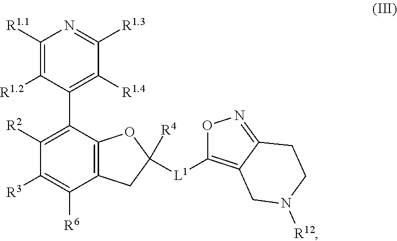

wherein R^{1.1}, R^{1.2}, R^{1.3}, R^{1.4}, R², R³, R⁴, R⁶, L¹, and R¹² are as described herein, including embodiments.

In embodiments, the compound has structural Formula (IV):

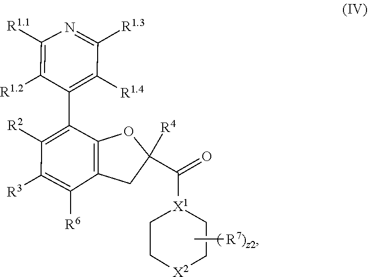

wherein X, X², R^{1.1}, R^{1.2}, R^{1.3}, R^{1.4}, R², R³, R⁴, R⁶, R⁷, and z2 are as described herein, including embodiments.

In embodiments, the compound has structural Formula (V):

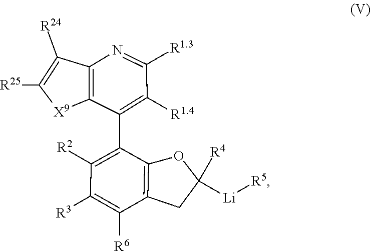

wherein R²⁴, R²⁵, R^{1.3}, R^{1.4}, R², R³, R⁴, R⁶, L¹, and R⁵ are as described herein, including embodiments. R^{1.3} and R^{1.4} can be joined together to form a substituted or unsubstituted cycloalkyl, substituted or unsubstituted heterocycloalkyl, substituted or unsubstituted aryl, or substituted or unsubstituted heteroaryl. X⁹ is —O—, —S—, or —N(R³³)—. R³³ is independently hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃₃H, —SOᵥ₃₃NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the compound has structural Formula (VI):

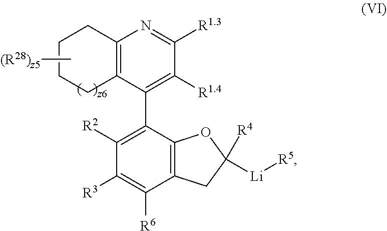

wherein R^{1.3}, R^{1.4}, R², R³, R⁴, R⁶, L¹, and R⁵ are as described herein, including embodiments. z⁵ is an integer from 0 to 8. z6 is 0 or 1. R²⁸ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₈H, —SOᵥ₂₈NH₂, —NHNH₂, —ONH₂, —NHC(O)NHNH₂, —NHC(O)NH₂, —N(O)ₘ₂₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, L¹ is a bond, —O—, —S—, —NH—, —C(O)—, —C(O)NH, —C(O)O—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene). In embodiments, L¹ is a bond, —S—, —NH—, —C(O)—, —C(O)NH, —C(O)O—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene). In embodiments, L¹ is a bond. In embodiments, L¹ is —O—. In embodiments, L¹ is —S—. In embodiments, L¹ is —NH—. In embodiments, L¹ is —C(O)—. In embodiments, L¹ is —C(O)NH. In embodiments, L¹ is —C(O)O—.

In embodiments, L¹ is substituted or unsubstituted alkylene (e.g., C₁-C₈ alkylene, C₁-C₆ alkylene, or C₁-C₄ alkylene). In embodiments, L¹ is substituted alkylene (e.g., C₁-C₈ alkylene, C₁-C₆ alkylene, or C₁-C₄ alkylene). In embodiments, L¹ is an unsubstituted alkylene (e.g., C₁-C₈ alkylene, C₁-C₆ alkylene, or C₁-C₄ alkylene). In embodiments, L¹ is substituted or unsubstituted heteroalkylene (e.g., 2 to 8 membered heteroalkylene, 2 to 6 membered heteroalkylene, or 2 to 4 membered heteroalkylene). In embodiments, L¹ is substituted heteroalkylene (e.g., 2 to 8 membered heteroalkylene, 2 to 6 membered heteroalkylene, or 2 to 4 membered heteroalkylene). In embodiments, L¹ is an unsubstituted heteroalkylene (e.g., 2 to 8 membered heteroalkylene, 2 to 6 membered heteroalkylene, or 2 to 4 membered heteroalkylene). In embodiments, L¹ is substituted or unsubstituted cycloalkylene (e.g., C₃-C₈ cycloalkylene, C₃-C₆ cycloalkylene, or C₅-C₆ cycloalkylene). In embodiments, L¹ is substituted cycloalkylene (e.g., C₃-C₈ cycloalkylene, C₃-C₆ cycloalkylene, or C₅-C₆ cycloalkylene). In embodiments, L¹ is an unsubstituted cycloalkylene (e.g., C₃-C₈ cycloalkylene, C₃-C₆ cycloalkylene, or C₅-C₆ cycloalkylene). In embodiments, L¹ is substituted or unsubstituted heterocycloalkylene (e.g., 3 to 8 membered heterocycloalkylene, 3 to 6 membered heterocycloalkylene, or 5 to 6 membered heterocycloalkylene). In embodiments, L¹ is substituted heterocycloalkylene (e.g., 3 to 8 membered heterocycloalkylene, 3 to 6 membered heterocycloalkylene, or 5 to 6 membered heterocycloalkylene). In embodiments, L¹ is an unsubstituted heterocycloalkylene (e.g., 3 to 8 membered heterocycloalkylene, 3 to 6 membered heterocycloalkylene, or 5 to 6 membered heterocycloalkylene). In embodiments, L¹ is substituted or unsubstituted arylene (e.g., C₆-C₁₀ arylene, C₁₀ arylene, or phenylene). In embodiments, L¹ is substituted arylene (e.g., C₆-C₁₀ arylene, C₁₀ arylene, or phenylene). In embodiments, L¹ is an unsubstituted arylene (e.g., C₆-C₁₀ arylene, C₁₀ arylene, or phenylene). In embodiments, L¹ is substituted or unsubstituted heteroarylene (e.g., 5 to 10 membered heteroarylene, 5 to 9 membered heteroarylene, or 5 to 6 membered heteroarylene). In embodiments, L¹ is substituted heteroarylene (e.g., 5 to 10 membered heteroarylene, 5 to 9 membered heteroarylene, or 5 to 6 membered heteroarylene). In embodiments, L¹ is an unsubstituted heteroarylene (e.g., 5 to 10 membered heteroarylene, 5 to 9 membered heteroarylene, or 5 to 6 membered heteroarylene).

In embodiments, L¹ is a bond or —C(O)—. In embodiments, L¹ is a bond. In embodiments, L¹ is —C(O)—. In embodiments, L¹ is a substituted C₁-C₄ alkylene. In embodiments, L¹ is a substituted methylene. In embodiments, L¹ is a substituted C₂ alkylene. In embodiments, L¹ is a substituted C₃ alkylene. In embodiments, L¹ is a substituted C₄ alkylene. In embodiments, L¹ is an unsubstituted C₁-C₄ alkylene. In embodiments, L¹ is an unsubstituted methylene. In embodiments, L¹ is an unsubstituted C₂ alkylene. In embodiments, L¹ is an unsubstituted C₃ alkylene. In embodiments, L¹ is an unsubstituted C₄ alkylene.

In embodiments, L¹ is a bond or —C(O)—; two R¹ substituents bonded to adjacent carbon atoms are joined to form a 5-membered substituted or unsubstituted heteroaryl or a 6-membered substituted or unsubstituted aryl; R² and R³ are independently hydrogen or halogen; R⁴ is hydrogen or an unsubstituted alkyl; and R⁶ is hydrogen.

In embodiments, L² is a bond, —O—, —S—, —NH—, —C(O)—, —C(O)NH, —C(O)O—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene). In embodiments, L² is a bond. In embodiments, L² is —O—. In embodiments, L² is —S—. In embodiments, L² is —NH—. In embodiments, L² is —C(O)—. In embodiments, L² is —C(O)NH. In embodiments, L² is —C(O)O—.

In embodiments, L² is substituted or unsubstituted alkylene (e.g., C₁-C₈ alkylene, C₁-C₆ alkylene, or C₁-C₄ alkylene). In embodiments, L² is substituted alkylene (e.g., C₁-C₈ alkylene, C₁-C₆ alkylene, or C₁-C₄ alkylene). In embodiments, L² is an unsubstituted alkylene (e.g., C₁-C₈ alkylene, C₁-C₆ alkylene, or C₁-C₄ alkylene). In embodiments, L² is substituted or unsubstituted heteroalkylene (e.g., 2 to 8 membered heteroalkylene, 2 to 6 membered heteroalkylene, or 2 to 4 membered heteroalkylene). In embodiments, L² is substituted heteroalkylene (e.g., 2 to 8 membered heteroalkylene, 2 to 6 membered heteroalkylene, or 2 to 4 membered heteroalkylene). In embodiments, L² is an unsubstituted heteroalkylene (e.g., 2 to 8 membered heteroalkylene, 2 to 6 membered heteroalkylene, or 2 to 4 membered heteroalkylene). In embodiments, L² is substituted or unsubstituted cycloalkylene (e.g., C₃-C₈ cycloalkylene, C₃-C₆ cycloalkylene, or C₅-C₆ cycloalkylene). In embodiments, L² is substituted cycloalkylene (e.g., C₃-C₈ cycloalkylene, C₃-C₆ cycloalkylene, or C₅-C₆ cycloalkylene). In embodiments, L² is an unsubstituted cycloalkylene (e.g., C₃-C₈ cycloalkylene, C₃-C₆ cycloalkylene, or C₅-C₆ cycloalkylene). In embodiments, L² is substituted or unsubstituted heterocycloalkylene (e.g., 3 to 8 membered heterocycloalkylene, 3 to 6 membered heterocycloalkylene, or 5 to 6 membered heterocycloalkylene). In embodiments, L² is substituted heterocycloalkylene (e.g., 3 to 8 membered heterocycloalkylene, 3 to 6 membered heterocycloalkylene, or 5 to 6 membered heterocycloalkylene). In embodiments, L² is an unsubstituted heterocycloalkylene (e.g., 3 to 8 membered heterocycloalkylene, 3 to 6 membered heterocycloalkylene, or 5 to 6 membered heterocycloalkylene). In embodiments, L² is substituted or unsubstituted arylene (e.g., C₆-C₁₀ arylene, C₁₀ arylene, or phenylene). In embodiments, L² is substituted arylene (e.g., C₆-C₁₀ arylene, C₁₀ arylene, or phenylene). In embodiments, L² is an unsubstituted arylene (e.g., C₆-C₁₀ arylene, C₁₀ arylene, or phenylene). In embodiments, L² is substituted or unsubstituted heteroarylene (e.g., 5 to 10 membered heteroarylene, 5 to 9 membered heteroarylene, or 5 to 6 membered heteroarylene). In embodiments, L² is substituted heteroarylene (e.g., 5 to 10 membered heteroarylene, 5 to 9 membered heteroarylene, or 5 to 6 membered heteroarylene). In embodiments, L² is an unsubstituted heteroarylene (e.g., 5 to 10 membered heteroarylene, 5 to 9 membered heteroarylene, or 5 to 6 membered heteroarylene).

In embodiments, Ring A-(R¹)_{z1} is

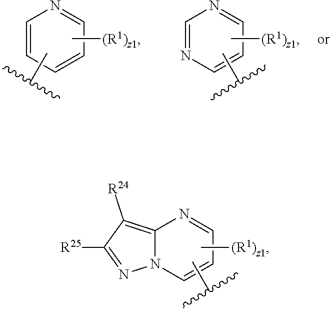

wherein R¹, z1, R²⁴, and R²⁵ are as described herein. In embodiments, Ring A-(R¹)_{z1} is

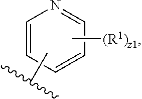

wherein R¹ and z1 are as described herein. In embodiments, Ring A-(R¹)_{z1} is

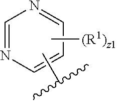

wherein R¹ and z1 are as described herein. In embodiments, Ring A-(R¹)_{z1} is

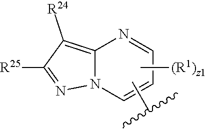

wherein R¹, z1, R²⁴, and R²⁵ are as described herein.

In embodiments, Ring A is

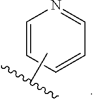

In embodiments, Ring A is

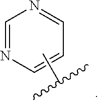

In embodiments, Ring A is

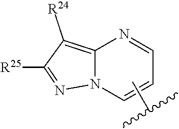

wherein R²⁴ and R²⁵ are as described herein.

In embodiments, Ring A is

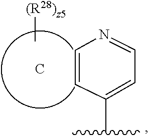

wherein R²⁸ and z5 are as described herein, including embodiments. Ring C is cycloalkyl, heterocycloalkyl, aryl, or heteroaryl. It is understood when z5 is equal or greater than 1, Ring C is substituted (e.g., independently substituted with a R²⁸ substituent). When z5 is 0, it is understood Ring C is unsubstituted.

In embodiments, Ring C is aziridinyl, 2H-azirinyl, oxiranyl, or thiiranyl. In embodiments, Ring C is azetidinyl, azetyl, oxetanyl, or thietanyl. In embodiments, Ring C is pyrrolidinyl, pyrrolyl, pyrazolidinyl, imidazolidinyl, pyrazolyl, imidazolyl, triazolyl, or tetrazolyl. In embodiments, Ring C is tetrahydrofuran, furan, 1,3-dioxolanyl, tetrahydrothiofuranyl, thiophenyl, oxazolyl, isoxazolyl, isothiazolyl, thiazolyl, or oathiolanyl. In embodiments, Ring C is sulfolanyl, thiazolidinyldionyl, succinimidyl, 2-oxazolidone, or hydantoin. In embodiments, Ring C is piperidinyl, pyridinyl, piperazinyl, pyridazinyl, pyrimidinyl, pyrazinyl, triazinyl, tetrahydropyran, pyran, pyrylium, 1,4-dioxanyl, or 1,4 dioxinyl. In embodiments, Ring C is thianyl, thiopyran, dithianyl, trithianyl, morpholinyl, or oxazinyl. In embodiments, Ring C is indenyl, indolinyl, indolyl, isoindolyl, indolizinyl, benzimidazolyl, or azaindolyl.

In embodiments, -Ring A-(R)_{z1} is

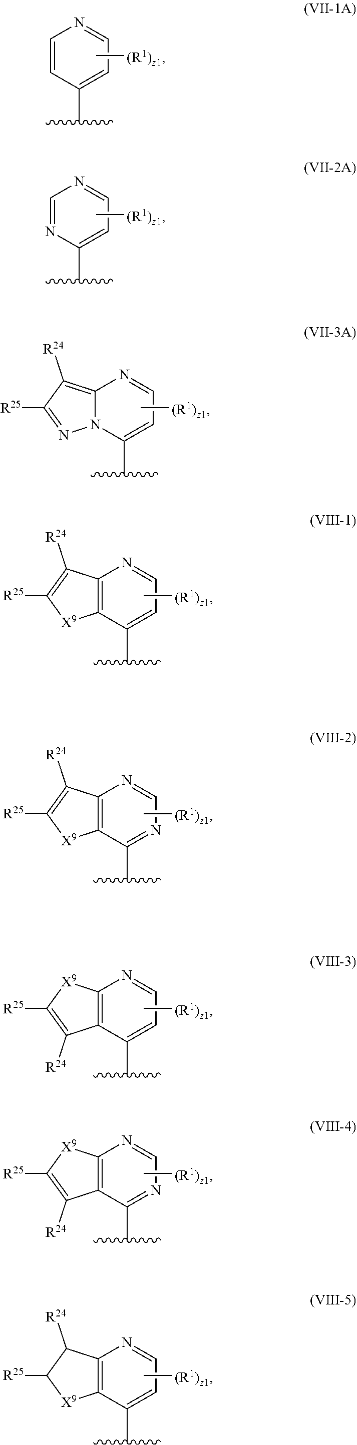

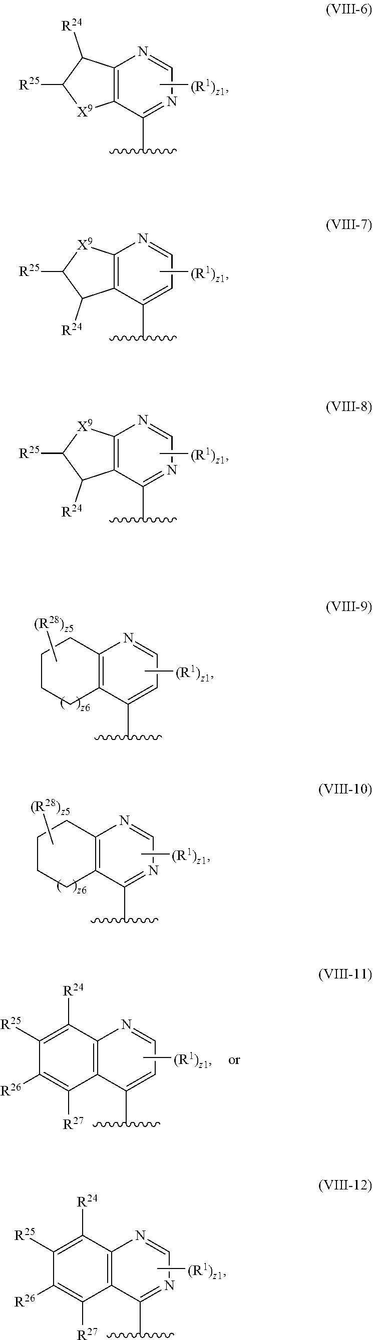

wherein R¹, z1, R²⁴, and R²⁵ are as described herein.

In embodiments, -Ring A-(R¹)_{z1} is:

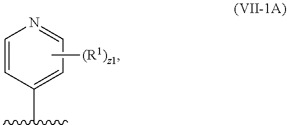

wherein R¹ and z1 are as described herein. In embodiments, -Ring A-(R¹)_{z1} is:

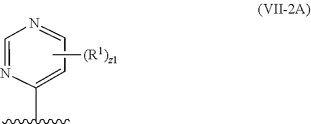

wherein R¹ and z1 are as described herein. In embodiments, -Ring A-(R¹)_{z1} is:

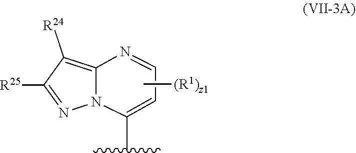

wherein R¹, z1, R²⁴, and R²⁵ are as described herein. In embodiments, -Ring A-(R¹)_{z1} is:

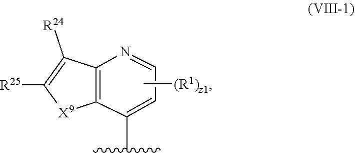

wherein R¹, z1, X⁹, R²⁴, and R²⁵ are as described herein. In embodiments, -Ring A-(R¹)_{z1} is:

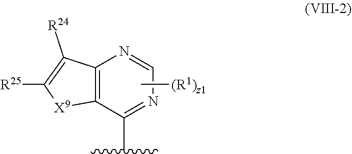

wherein R¹, z1, X⁹, R²⁴, and R²⁵ are as described herein. In embodiments, -Ring A-(R¹)_{z1} is:

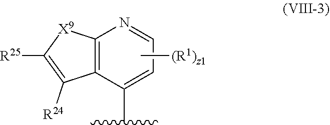

wherein R¹, z1, X⁹, R²⁴, and R²⁵ are as described herein. In embodiments, -Ring A-(R¹)_{z1} is:

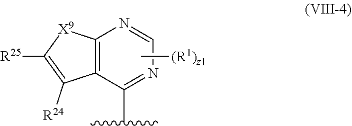

wherein R¹, z1, X⁹, R²⁴, and R²⁵ are as described herein. In embodiments, -Ring A-(R)_{z1} is:

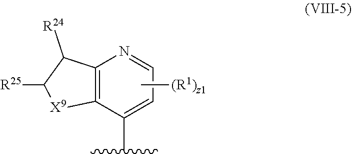

wherein R¹, z1, X⁹, R²⁴, and R²⁵ are as described herein. In embodiments, -Ring A-(R¹)_{z1} is:

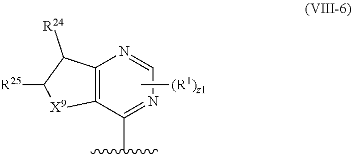

wherein R¹, z1, X⁹, R²⁴, and R²⁵ are as described herein. In embodiments, -Ring A-(R¹)_{z1} is:

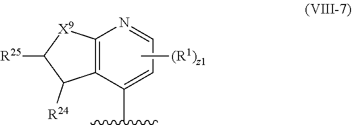

wherein R¹, z1, X⁹, R²⁴, and R²⁵ are as described herein. In embodiments, -Ring A-(R¹)_{z1} is:

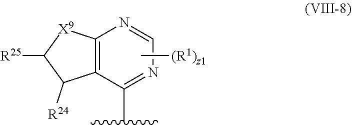

wherein R¹, z1, X⁹, R²⁴, and R²⁵ are as described herein. In embodiments, -Ring A-(R¹)_{z1} is:

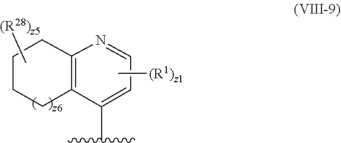

wherein R¹, z1, z6, z5, and R²⁸ are as described herein. In embodiments, -Ring A-(R¹)_{z1} is:

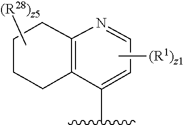

wherein R¹, z1, z5, and R²⁸ are as described herein. In embodiments, -Ring A-(R¹)_{z1} is:

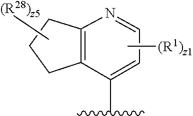

wherein R¹, z1, z5, and R²⁸ are as described herein. In embodiments, -Ring A-(R¹)_{z1} is:

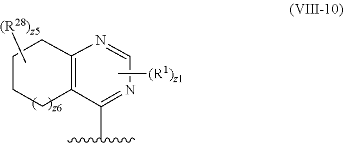

wherein R¹, z1, z6, z5, and R²⁸ are as described herein. In embodiments, -Ring A-(R¹)_{z1} is:

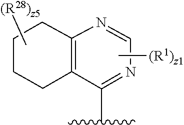

wherein R¹, z1, z5, and R²⁸ are as described herein. In embodiments, -Ring A-(R¹)_{z1} is:

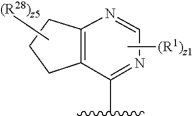

wherein R¹, z1, z5, and R²⁸ are as described herein. In embodiments, -Ring A-(R¹)_{z1} is:

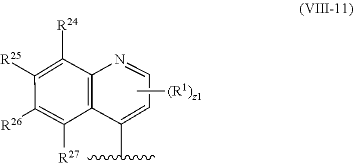

wherein R¹, z1, R²⁴, and R²⁵ are as described herein. In embodiments, -Ring A-(R¹)_{z1} is:

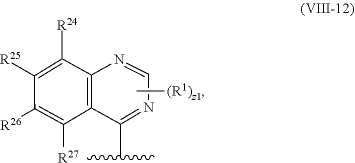

wherein R¹, z1, R²⁴, and R²⁵ are as described herein.

The symbol X⁹ is —O—, —S— or —N(R³³)—. R²⁶ is hydrogen, halogen, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SOₙ₂₆R^{26A}, —SOᵥ₂₆NR^{26B}R^{26C}, —NHNR^{26B}R^{26C}, —ONR^{26B}R^{26C}, —NHC(O)NHNR^{26B}R^{26C}, —NHC(O)NR^{26B}R^{26C}, —N(O)ₘ₂₆, —NR^{26B}R^{26C}, —C(O)R^{26D}, —C(O)OR^{26D}, —C(O)NR^{26B}R^{26C}, —OR^{26A}, —NR^{26B}SO₂R^{26A}, —NR^{26B}C(O)R^{26D}, —NR^{26B}C(O)OR^{26D}, —NR^{26B}OR^{26D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R²⁷ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₇R^{27A}, —SOᵥ₂₇NR^{27B}R^{27C}, —NHNR^{27B}R^{27C}, —ONR^{27B}R^{27C}, —NHC(O)NHNR^{27B}R^{27C}, —NHC(O)NR^{27B}R^{27C}, —N(O)ₘ₂₇, —NR^{27B}R^{27C}, —C(O)R^{27D}, —C(O)OR^{27D}, —C(O)NR^{27B}R^{27C}, —OR^{27A}, —NR^{27B}SO₂R^{27A}, —NR²⁷BC(O)R^{27D}, —NR²⁷BC(O)OR^{27D}, —NR^{27B}OR^{27D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. R²⁸ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₈H, —SOᵥ₂₈NH₂, —NHNH₂, —ONH₂, —NHC(O)NHNH₂, —NHC(O)NH₂, —N(O)ₘ₂₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³³ is independently hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃₃H, —SOᵥ₃₃NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{26A}, R^{26B}, R^{26C}, R^{26D}, R^{27A}, R^{27B}, R^{27C} and R^{27D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{26B} and R^{26C}, or R^{27B} and R^{27C} substituents bonded to the same nitrogen atom may optionally be joined to form a substituted or unsubstituted heterocycloalkyl or substituted or unsubstituted heteroaryl. The symbol z5 is an integer from 0 to 8. The symbol z6 is 0 or 1. The symbols n26, n27, n28, and n33 are independently an integer from 0 to 4. The symbols m26, m27, m28, m33, v26, v27, v28, and v33 are independently 1 or 2.

In embodiments, -Ring A-(R¹)_{z1} is:

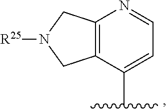

wherein R²⁵ is as described herein. In embodiments, -Ring A-(R¹)_{z} is:

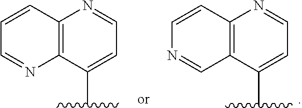 [SMILES: *c1ccnc2cccnc12 ; *c1ccnc2ccncc12]

In embodiments, -Ring A-(R¹)_{z1} is:

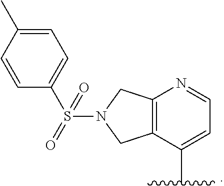 [SMILES: *c1ccnc2c1CN(S(=O)(=O)c1ccc(C)cc1)C2]

In embodiments, -Ring A-(R¹)_{z1} is:

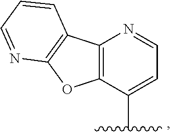 [SMILES: *c1ccnc2c1oc1ncccc12]

In embodiments, -Ring A-(R¹)_{z1} is:

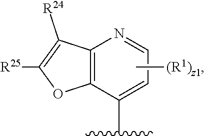

wherein R¹, z1, R²⁴, and R²⁵ are as described herein, including embodiments. In embodiments, -Ring A-(R¹)_{z1} is:

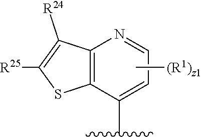

wherein R¹, z1, R²⁴, and R²⁵ are as described herein, including embodiments. In embodiments, -Ring A-(R¹)_{z1} is:

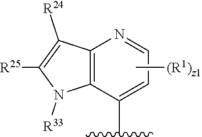

wherein R¹, z1, R³³, R²⁴, and R²⁵ are as described herein, including embodiments.

In embodiments, -Ring A-(R¹)_{z1} is:

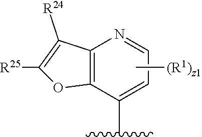

wherein R¹, z1, R²⁴, and R²⁵ are as described herein, including embodiments. In embodiments, -Ring A-(R¹)_{z1} is:

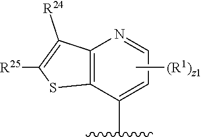

wherein R¹, R²⁴, and R²⁵ are as described herein, including embodiments. In embodiments, -Ring A-(R¹)_{z1} is:

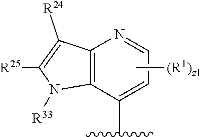

wherein R, z1, R³³, R²⁴, and R²⁵ are as described herein, including embodiments.

In embodiments, -Ring A-(R)_{z1} is

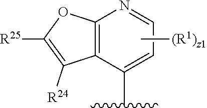

wherein R¹, z1, R²⁴, and R²⁵ are as described herein, including embodiments. In embodiments, -Ring A-(R¹)_{z1} is:

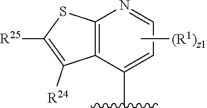

wherein R¹, z1, R²⁴, and R²⁵ are as described herein, including embodiments. In embodiments, -Ring A-(R¹)_{z1} is:

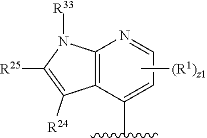

wherein R, z1, R³³, R²⁴, and R²⁵ are as described herein, including embodiments. In embodiments, -Ring A-(R)_{z1} is:

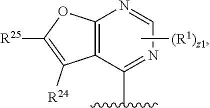

wherein R¹, z1, R²⁴, and R²⁵ are as described herein, including embodiments. In embodiments, -Ring A-(R)_{z} is:

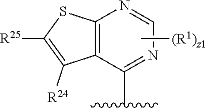

wherein R¹, z1, R²⁴, and R²⁵ are as described herein, including embodiments. In embodiments, -Ring A-(R¹)_{z1} is:

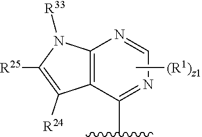

wherein R¹, z1, R³³, R²⁴, and R²⁵ are as described herein, including embodiments.

In embodiments, -Ring A-(R¹)_{z1} is:

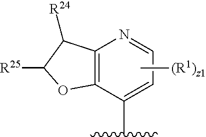

wherein R¹, z1, R²⁴, and R²⁵ are as described herein, including embodiments. In embodiments, -Ring A-(R¹)_{z1} is:

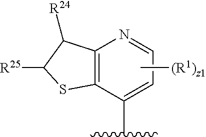

wherein R¹, z1, R²⁴, and R²⁵ are as described herein, including embodiments. In embodiments, -Ring A-(R)_{z1} is

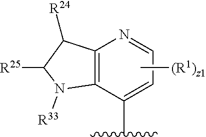

wherein R¹, z1, R³³, R²⁴, and R²⁵ are as described herein, including embodiments.

In embodiments, -Ring A-(R)_{z1} is:

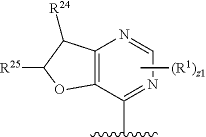

wherein R¹, z1, R²⁴, and R²⁵ are as described herein, including embodiments. In embodiments, -Ring A-(R¹)_{z1} is:

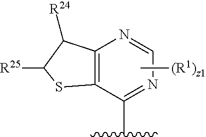

wherein R¹, z1, R²⁴, and R²⁵ are as described herein, including embodiments. In embodiments, -Ring A-(R)_{z1} is:

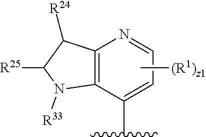

wherein R¹, z1, R³³, R²⁴, and R²⁵ are as described herein, including embodiments.

In embodiments, -Ring A-(R¹)_{z1} is:

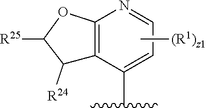

wherein R¹, z1, R²⁴, and R²⁵ are as described herein, including embodiments. In embodiments, -Ring A-(R¹)_{z1} is:

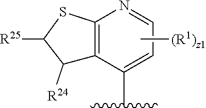

wherein R¹, z1, R²⁴, and R²⁵ are as described herein, including embodiments. In embodiments, -Ring A-(R)_{z1} is:

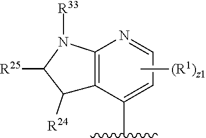

wherein R¹, z1, R³³, R²⁴, and R²⁵ are as described herein, including embodiments. In embodiments, -Ring A-(R¹)_{z1} is

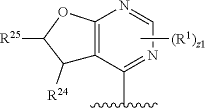

wherein R¹, z1, R²⁴, and R²⁵ are as described herein, including embodiments. In embodiments, -Ring A-(R¹)_{z1} is:

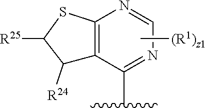

wherein R¹, z1, R²⁴, and R²⁵ are as described herein, including embodiments. In embodiments, -Ring A-(R¹)_{z1} is:

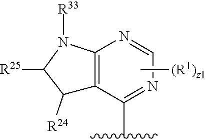

wherein R¹, z1, R³³, R²⁴, and R²⁵ are as described herein, including embodiments.

In embodiments, R^{1.1} and R^{1.2} are joined together and with the pyridyl ring (i.e., Ring A) to which they are attached form: R

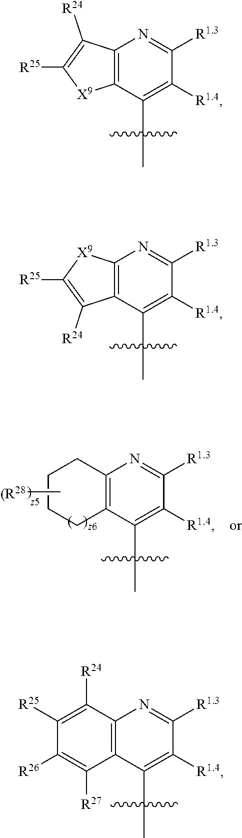

wherein R²⁴, R²⁵, X⁹, R^{1.3}, R^{1.4}, R²⁸, z5, z6, R²⁶, and R²⁷ are as described herein, including embodiments.

In embodiments, R^{1.1} and R^{1.2} are joined together and with the pyridyl ring to which they are attached form:

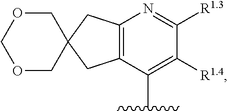

wherein R^{1.3} and R^{1.4} are as described herein, including embodiments. In embodiments, R^{1.1} and R^{1.2} are joined together and with the pyridyl ring to which they are attached form:

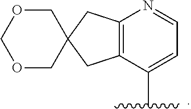 [SMILES: *c1ccnc2c1CC1(COCOC1)C2]

In embodiments, R^{1.1} and R^{1.2} are joined together and with the pyridyl ring to which they are attached form:

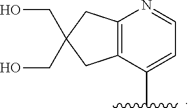 [SMILES: *c1ccnc2c1CC(CO)(CO)C2]

In embodiments, R^{1.1} and R^{1.2} are joined together and with the pyridyl ring to which they are attached form:

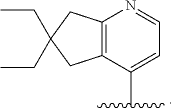 [SMILES: *c1ccnc2c1CC(CC)(CC)C2]

In embodiments, R^{1.1} and R^{1.2} are joined together and with the pyridyl ring to which they are attached form:

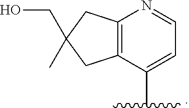 [SMILES: *c1ccnc2c1CC(C)(CO)C2]

In embodiments, R^{1.1} and R^{1.2} are joined together and with the pyridyl ring to which they are attached form:

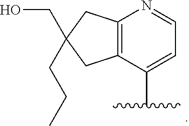 [SMILES: *c1ccnc2c1CC(CO)(CCC)C2]

In embodiments, Ring B is substituted or unsubstituted heterocycloalkyl, substituted or unsubstituted aryl, or substituted or unsubstituted heteroaryl.

In embodiments, Ring B is a substituted or unsubstituted monovalent succinimide moiety, substituted or unsubstituted monovalent hydantoin moiety, substituted or unsubstituted monovalent oxohydantoin moiety, substituted or unsubstituted monovalent thiohydantoin moiety, substituted or unsubstituted monovalent pyrrolidone moiety, substituted or unsubstituted monovalent phthalimide moiety, substituted or unsubstituted monovalent oxazolidinone moiety, or substituted or unsubstituted monovalent cyclic urea moiety.

In embodiments, Ring B is

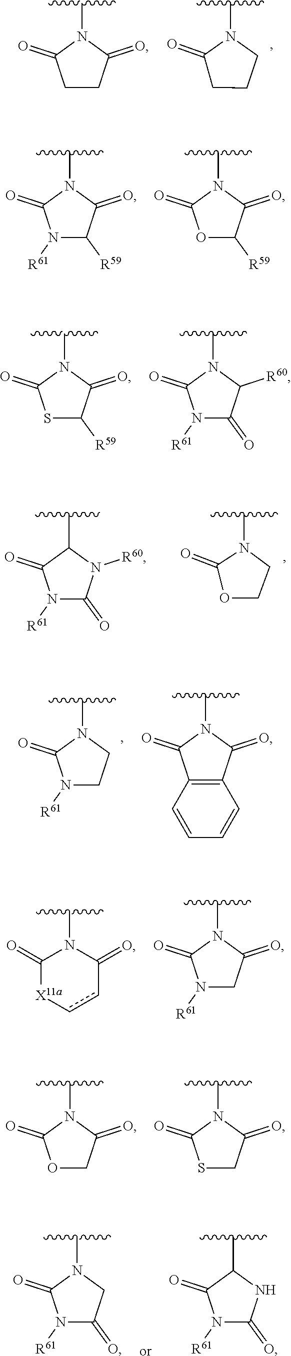

wherein R⁵⁹ is as described herein, including embodiments. X¹¹ is —C(R⁶¹)(R⁶²)— or —N(R⁶¹)—. The symbol [symbol] is a single bond or double bond. R⁶⁰ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₆₀R^{60A}, —SOᵥ₆₀NR^{60B}R^{60C}, —NHNR^{60B}R^{60C}, —ONR^{60B}R^{60C}, —NHC(O)NHNR^{60B}R^{60C}, —NHC(O)NR^{60B}R^{60C}, —N(O)ₘ₆₀, —NR^{60B}R^{60C}, —C(O)R^{60D}, —C(O)OR^{60D}, —C(O)NR^{60B}R^{60C}, —OR^{60A}, —NR^{60B}SO₂R^{60A}, —NR^{60B}C(O)R^{60D}, —NR^{60B}C(O)OR^{60D}, —NR^{60B}OR^{60D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶¹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₆₁R^{61A}, —SOᵥ₆₁NR^{61B}R^{61C}, —NHNR^{61B}R^{61C}, —ONR^{61B}R^{61C}, —NHC(O)NHNR^{61B}R^{61C}, —NHC(O)NR^{61B}R^{61C}, —N(O)ₘ₆₁, —NR^{61B}R^{61C}, —C(O)R^{61D}, —C(O)OR^{61D}, —C(O)NR^{61B}R^{61C}, —OR^{61A}, —NR^{61B}SO₂R^{61A}, —NR^{61B}C(O)R^{61D}, —NR^{61B}C(O)OR^{61D}, —NR^{61B}OR^{61D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶² is independently hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₆₂H, —SOᵥ₆₂NH₂, —NHNH₂, —ONH₂, —NHC(O)NHNH₂, —NHC(O)NH₂, —N(O)ₘ₆₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶¹ and R⁶² are optionally together oxo. R^{60A}, R^{6B}, R^{60C}, and R^{60D}, R^{61A}, R^{61B}, R^{61C} and R^{61D} are independently hydrogen, halogen, —CF₃, —CCl₃, —CBr₃, —Cl₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{60B} and R^{60C} substituents bonded to the same nitrogen atom may optionally be joined to form a substituted or unsubstituted heterocycloalkyl or substituted or unsubstituted heteroaryl. R^{61B} and R^{61C} substituents bonded to the same nitrogen atom may optionally be joined to form a substituted or unsubstituted heterocycloalkyl or substituted or unsubstituted heteroaryl. The symbols n60, n61 and n62 are independently an integer from 0 to 4. The symbols m60, m61, m62, v60, v61 and v62 are independently 1 or 2.

In embodiments, -Ring B—(R⁵⁹)z9 is:

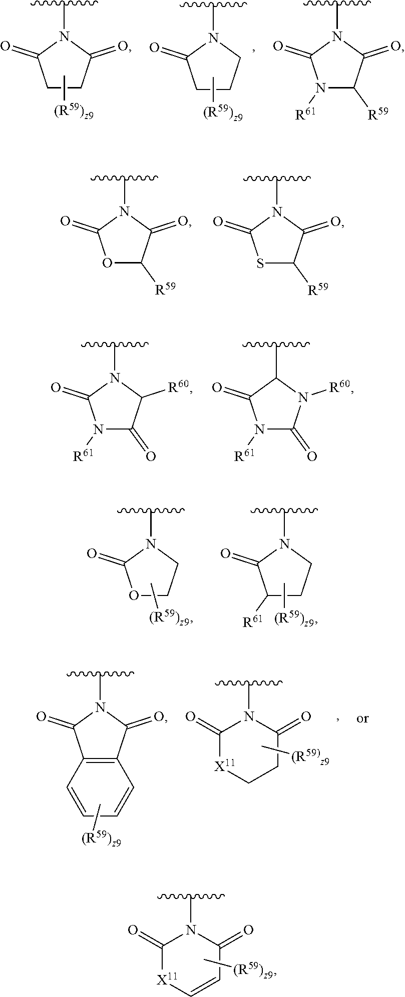

wherein R⁵⁹, R⁶⁰, and R⁶¹ is as described herein, including embodiments.

In embodiments, -Ring B—(R⁵⁹)z9 is:

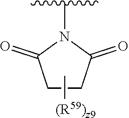

wherein R⁵⁹ is as described herein, including embodiments. In embodiments, -Ring B—(R⁵⁹)z9 is:

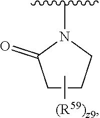

wherein R⁵⁹ is as described herein, including embodiments. In embodiments, -Ring B—(R⁵⁹)z9 is

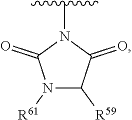

wherein R⁵⁹ and R⁶¹ are as described herein, including embodiments. In embodiments, -Ring B—(R⁵⁹)z9 is:

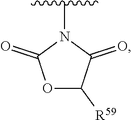

wherein R⁵⁹ is as described herein, including embodiments. In embodiments, -Ring B—(R⁵⁹)z9 is:

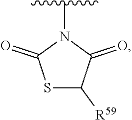

wherein R⁵⁹ is as described herein, including embodiments. In embodiments, -Ring B—(R⁵⁹)z9 is:

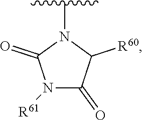

wherein R⁶⁰ and R⁶¹ are as described herein, including embodiments. In embodiments, -Ring B—(R⁵⁹)z9 is:

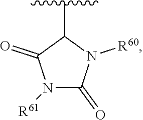

wherein R⁶⁰ and R⁶¹ are as described herein, including embodiments. In embodiments, -Ring B—(R⁵⁹)z9 is:

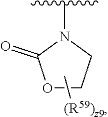

wherein R⁵⁹ is as described herein, including embodiments. In embodiments, -Ring B—(R⁵⁹)z9 is:

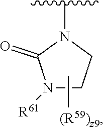

wherein R⁵⁹ and R⁶¹ are as described herein, including embodiments. In embodiments, -Ring B-(R⁵⁹)z9 is:

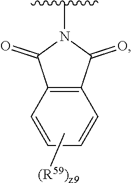

wherein R⁵⁹ is as described herein, including embodiments. In embodiments, -Ring B—(R⁵⁹)z9 is:

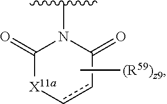

wherein R⁵⁹ is as described herein, including embodiments. In embodiments, -Ring B—(R⁵⁹)z9 is:

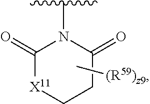

wherein R⁵⁹ is as described herein, including embodiments. In embodiments, -Ring B—(R⁵⁹)z9 is:

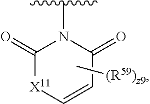

wherein R⁵⁹ as described herein, including embodiments. In embodiments, -Ring B is:

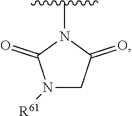

wherein R⁶¹ is as described herein, including embodiments. In embodiments, -Ring B is:

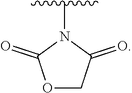

In embodiments, -Ring B is:

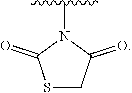

In embodiments, -Ring B is

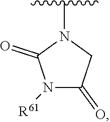

wherein R⁶¹ is as described herein, including embodiments. In embodiments, -Ring B is or

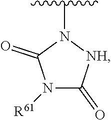

wherein R⁶¹ is as described herein, including embodiments.

In embodiments, Ring B is

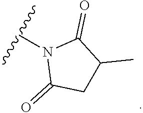 [SMILES: *N1C(=O)CC(C)C1=O]

In embodiments, Ring B is

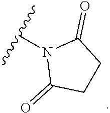 [SMILES: *N1C(=O)CCC1=O]

In embodiments, Ring B is

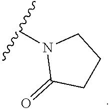 [SMILES: *N1CCCC1=O]

In embodiments, Ring B is

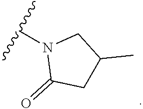 [SMILES: *N1CC(C)CC1=O]

In embodiments, Ring B is

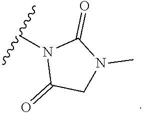 [SMILES: *N1C(=O)CN(C)C1=O]

In embodiments, Ring B is

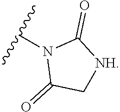 [SMILES: *N1C(=O)CNC1=O]

In embodiments, Ring B is

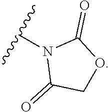 [SMILES: *N1C(=O)COC1=O]

In embodiments, Ring B is

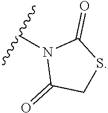 [SMILES: *N1C(=O)CSC1=O]

In embodiments, Ring B is

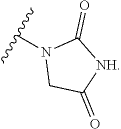 [SMILES: *N1CC(=O)NC1=O]

In embodiments, Ring B is

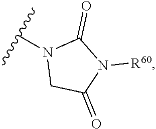

wherein R⁶⁰ is as described herein, including embodiments. In embodiments, Ring B is

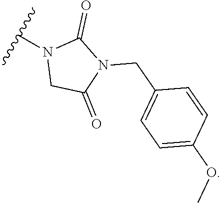 [SMILES: *N1CC(=O)N(Cc2ccc(OC)cc2)C1=O]

In embodiments, Ring B is

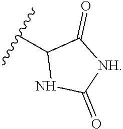 [SMILES: *C1NC(=O)NC1=O]

In embodiments, Ring B is

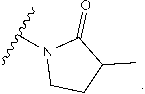 [SMILES: *N1CCC(C)C1=O]

In embodiments, Ring B is

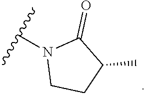 [SMILES: *N1CC[C@@H](C)C1=O]

In embodiments, Ring B is

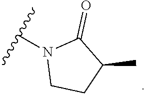 [SMILES: *N1CC[C@H](C)C1=O]

In embodiments, Ring B is

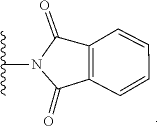 [SMILES: *N1C(=O)c2ccccc2C1=O]

In embodiments, Ring B is

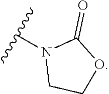 [SMILES: *N1CCOC1=O]

In embodiments, Ring B is

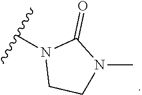 [SMILES: *N1CCN(C)C1=O]

In embodiments, Ring B is

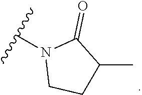 [SMILES: *N1CCC(C)C1=O]

In embodiments, Ring B is

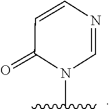 [SMILES: *n1cnccc1=O]

In embodiments, Ring B is

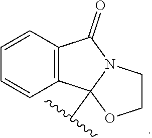 [SMILES: *C12OCCN1C(=O)c1ccccc12]

In embodiments, Ring B is

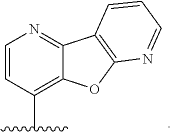 [SMILES: *c1ccnc2c1oc1ncccc12]

In embodiments, Ring B is

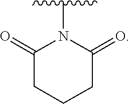 [SMILES: *N1C(=O)CCCC1=O]

In embodiments, Ring B is

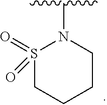 [SMILES: *N1CCCCS1(=O)=O]

In embodiments, Ring B is

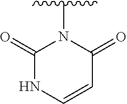 [SMILES: *n1c(=O)cc[nH]c1=O]

In embodiments, Ring B is

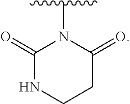 [SMILES: *N1C(=O)CCNC1=O]

In embodiments, Ring B is

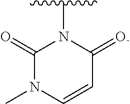 [SMILES: *n1c(=O)ccn(C)c1=O]

In embodiments, Ring B is

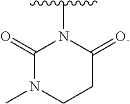 [SMILES: *N1C(=O)CCN(C)C1=O]

In embodiments, Ring B is unsubstituted phenyl. In embodiments, Ring B is unsubstituted pyridyl. In embodiments, Ring B is substituted phenyl. In embodiments, Ring B is

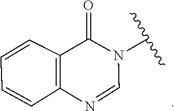 [SMILES: *n1cnc2ccccc2c1=O]

In embodiments, Ring B is

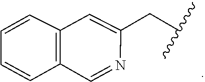 [SMILES: *Cc1cc2ccccc2cn1]

In embodiments, Ring B is

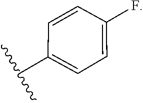 [SMILES: *c1ccc(F)cc1]

In embodiments, R¹ is independently halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —OCF₃, —OCCl₃, —OCBr₃, —OCI₃, —OCHF₂, —OCHCl₂, —OCHBr₂, —OCHI₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R¹ is independently halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁H, —SOᵥ₁NH₂, —NHNH₂, —ONH₂, —NHC(O)NHNH₂, —NHC(O)NH₂, —N(O)ₘ₁, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, or —CH₂I.

In embodiments, R¹ is independently halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁H, —SOᵥ₁NH₂, —NHNH₂, —ONH₂, —NHC(O)NHNH₂, —NHC(O)NH₂, —N(O)ₘ₁, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, two R¹ substituents bonded to adjacent carbon atoms may optionally be joined to form a substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, two R¹ substituents bonded to adjacent carbon atoms are joined to form a 5-membered substituted or unsubstituted heteroaryl or a 6-membered substituted or unsubstituted aryl. In embodiments, two R¹ substituents (e.g., R^{1.1} and R^{1.2} or R^{1.3} and R^{1.4}) bonded to adjacent carbon atoms may optionally be joined to form a substituted cycloalkyl (e.g., C₃-C₈ cycloalkyl, C₃-C₆ cycloalkyl, or C₅-C₆ cycloalkyl), substituted heterocycloalkyl (e.g., 3 to 8 membered heterocycloalkyl, 3 to 6 membered heterocycloalkyl, or 5 to 6 membered heterocycloalkyl), substituted aryl (e.g., C₆-C₁₀ aryl, C₁₀ aryl, or phenyl), or substituted heteroaryl (e.g., 5 to 10 membered heteroaryl, 5 to 9 membered heteroaryl, or 5 to 6 membered heteroaryl). In embodiments, two R¹ substituents (e.g., R^{1.1} and R^{1.2} or R^{1.3} and R^{1.4}) bonded to adjacent carbon atoms may optionally be joined to form an unsubstituted cycloalkyl (e.g., C₃-C₈ cycloalkyl, C₃-C₆ cycloalkyl, or C₅-C₆ cycloalkyl), an unsubstituted heterocycloalkyl (e.g., 3 to 8 membered heterocycloalkyl, 3 to 6 membered heterocycloalkyl, or 5 to 6 membered heterocycloalkyl), an unsubstituted aryl (e.g., C₆-C₁₀ aryl, C₁₀ aryl, or phenyl), or an unsubstituted heteroaryl (e.g., 5 to 10 membered heteroaryl, 5 to 9 membered heteroaryl, or 5 to 6 membered heteroaryl).

In embodiments, two R¹ substituents (e.g., R^{1.1} and R^{1.2} or R^{1.3} and R^{1.4}) bonded to adjacent carbon atoms may optionally be joined to form a substituted cycloalkyl (e.g., C₃-C₈ cycloalkyl, C₃-C₆ cycloalkyl, or C₅-C₆ cycloalkyl). In embodiments, two R¹ substituents (e.g., R^{1.1} and R^{1.2} or R^{1.3} and R^{1.4}) bonded to adjacent carbon atoms may optionally be joined to form a substituted heterocycloalkyl (e.g., 3 to 8 membered heterocycloalkyl, 3 to 6 membered heterocycloalkyl, or 5 to 6 membered heterocycloalkyl). In embodiments, two R¹ substituents (e.g., R^{1.1} and R^{1.2} or R^{1.3} and R^{1.4}) bonded to adjacent carbon atoms may optionally be joined to form a substituted aryl (e.g., C₆-C₁₀ aryl, C₁₀ aryl, or phenyl). In embodiments, two R¹ substituents (e.g., R^{1.1} and R^{1.2} or R^{1.3} and R^{1.4}) bonded to adjacent carbon atoms may optionally be joined to form a substituted heteroaryl (e.g., 5 to 10 membered heteroaryl, 5 to 9 membered heteroaryl, or 5 to 6 membered heteroaryl).

In embodiments, two R¹ substituents (e.g., R^{1.1} and R^{1.2} or R^{1.3} and R^{1.4}) bonded to adjacent carbon atoms may optionally be joined to form an unsubstituted cycloalkyl (e.g., C₃-C₈ cycloalkyl, C₃-C₆ cycloalkyl, or C₅-C₆ cycloalkyl). In embodiments, two R¹ substituents (e.g., R^{1.1} and R^{1.2} or R^{1.3} and R^{1.4}) bonded to adjacent carbon atoms may optionally be joined to form an unsubstituted heterocycloalkyl (e.g., 3 to 8 membered heterocycloalkyl, 3 to 6 membered heterocycloalkyl, or 5 to 6 membered heterocycloalkyl). In embodiments, two R¹ substituents (e.g., R^{1.1} and R^{1.2} or R^{1.3} and R^{1.4}) bonded to adjacent carbon atoms may optionally be joined to form an unsubstituted aryl (e.g., C₆-C₁₀ aryl, C₁₀ aryl, or phenyl). In embodiments, two R¹ substituents (e.g., R^{1.1} and R^{1.2} or R^{1.3} and R^{1.4}) bonded to adjacent carbon atoms may optionally be joined to form an unsubstituted heteroaryl (e.g., 5 to 10 membered heteroaryl, 5 to 9 membered heteroaryl, or 5 to 6 membered heteroaryl).

In embodiments, R¹ is R³⁵-substituted or unsubstituted alkyl (e.g., C₁-C₈ alkyl, C₁-C₆ alkyl, or C₁-C₄ alkyl). In embodiments, R¹ is R³⁵-substituted alkyl (e.g., C₁-C₈ alkyl, C₁-C₆ alkyl, or C₁-C₄ alkyl). In embodiments, R¹ is an unsubstituted alkyl (e.g., C₁-C₈ alkyl, C₁-C₆ alkyl, or C₁-C₄ alkyl).

In embodiments, R¹ is R³⁵-substituted or unsubstituted heteroalkyl (e.g., 2 to 8 membered heteroalkyl, 2 to 6 membered heteroalkyl, or 2 to 4 membered heteroalkyl). In embodiments, R¹ is R³⁵-substituted heteroalkyl (e.g., 2 to 8 membered heteroalkyl, 2 to 6 membered heteroalkyl, or 2 to 4 membered heteroalkyl). In embodiments, R¹ is an unsubstituted heteroalkyl (e.g., 2 to 8 membered heteroalkyl, 2 to 6 membered heteroalkyl, or 2 to 4 membered heteroalkyl).

In embodiments, R¹ is R³⁵-substituted or unsubstituted cycloalkyl (e.g., C₃-C₈ cycloalkyl, C₃-C₆ cycloalkyl, or C₅-C₆ cycloalkyl). In embodiments, R¹ is R³⁵-substituted cycloalkyl (e.g., C₃-C₈ cycloalkyl, C₃-C₆ cycloalkyl, or C₅-C₆ cycloalkyl). In embodiments, R¹ is an unsubstituted cycloalkyl (e.g., C₃-C₈ cycloalkyl, C₃-C₆ cycloalkyl, or C₅-C₆ cycloalkyl).

In embodiments, R¹ is R³⁵-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹ is R³⁵-substituted heterocycloalkyl (e.g., 3 to 8 membered heterocycloalkyl, 3 to 6 membered heterocycloalkyl, or 5 to 6 membered heterocycloalkyl). In embodiments, R¹ is an unsubstituted heterocycloalkyl (e.g., 3 to 8 membered heterocycloalkyl, 3 to 6 membered heterocycloalkyl, or 5 to 6 membered heterocycloalkyl).

In embodiments, R¹ is R³⁵-substituted or unsubstituted aryl (e.g., C₆-C₁₀ aryl, C₁₀ aryl, or phenyl). In embodiments, R¹ is R³⁵-substituted aryl (e.g., C₆-C₁₀ aryl, C₁₀ aryl, or phenyl). In embodiments, R¹ is an unsubstituted aryl (e.g., C₆-C₁₀ aryl, C₁₀ aryl, or phenyl).

In embodiments, R¹ is R³⁵-substituted or unsubstituted heteroaryl (e.g., 5 to 10 membered heteroaryl, 5 to 9 membered heteroaryl, or 5 to 6 membered heteroaryl). In embodiments, R¹ is R³⁵-substituted heteroaryl (e.g., 5 to 10 membered heteroaryl, 5 to 9 membered heteroaryl, or 5 to 6 membered heteroaryl). In embodiments, R¹ is an unsubstituted heteroaryl (e.g., 5 to 10 membered heteroaryl, 5 to 9 membered heteroaryl, or 5 to 6 membered heteroaryl).

In embodiments, R¹ is independently halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —CH₂I, R³⁵-substituted or unsubstituted alkyl (e.g., C₁-C₈ alkyl, C₁-C₆ alkyl, or C₁-C₄ alkyl), R³⁵-substituted or unsubstituted heteroalkyl (e.g., 2 to 8 membered heteroalkyl, 2 to 6 membered heteroalkyl, or 2 to 4 membered heteroalkyl), R³⁵-substituted or unsubstituted cycloalkyl (e.g., C₃-C₈ cycloalkyl, C₃-C₆ cycloalkyl, or C₅-C₆ cycloalkyl), R³⁵-substituted or unsubstituted heterocycloalkyl (e.g., 3 to 8 membered heterocycloalkyl, 3 to 6 membered heterocycloalkyl, or 5 to 6 membered heterocycloalkyl), R³⁵-substituted or unsubstituted aryl (e.g., C₆-C₁₀ aryl, C₁₀ aryl, or phenyl), or R³⁵-substituted or unsubstituted heteroaryl (e.g., 5 to 10 membered heteroaryl, 5 to 9 membered heteroaryl, or 5 to 6 membered heteroaryl).

R³⁵ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂F, —OCH₂Cl, —OCH₂Br, —CH₂I, R³⁶-substituted or unsubstituted alkyl (e.g., C₁-C₈ alkyl, C₁-C₆ alkyl, or C₁-C₄ alkyl), R³⁶-substituted or unsubstituted heteroalkyl (e.g., 2 to 8 membered heteroalkyl, 2 to 6 membered heteroalkyl, or 2 to 4 membered heteroalkyl), R³⁶-substituted or unsubstituted cycloalkyl (e.g., C₃-C₈ cycloalkyl, C₃-C₆ cycloalkyl, or C₅-C₆ cycloalkyl), R³⁶-substituted or unsubstituted heterocycloalkyl (e.g., 3 to 8 membered heterocycloalkyl, 3 to 6 membered heterocycloalkyl, or 5 to 6 membered heterocycloalkyl), R³⁶-substituted or unsubstituted aryl (e.g., C₆-C₁₀ aryl, C₁₀ aryl, or phenyl), or R³⁶-substituted or unsubstituted heteroaryl (e.g., 5 to 10 membered heteroaryl, 5 to 9 membered heteroaryl, or 5 to 6 membered heteroaryl).

In embodiments, R³⁵ is R³⁶-substituted or unsubstituted alkyl (e.g., C₁-C₈ alkyl, C₁-C₆ alkyl, or C₁-C₄ alkyl). In embodiments, R³⁵ is R³⁶-substituted alkyl (e.g., C₁-C₈ alkyl, C₁-C₆ alkyl, or C₁-C₄ alkyl). In embodiments, R³⁵ is an unsubstituted alkyl (e.g., C₁-C₈ alkyl, C₁-C₆ alkyl, or C₁-C₄ alkyl). In embodiments, R³⁵ is R³⁶-substituted or unsubstituted heteroalkyl (e.g., 2 to 8 membered heteroalkyl, 2 to 6 membered heteroalkyl, or 2 to 4 membered heteroalkyl). In embodiments, R³⁵ is R³⁶-substituted heteroalkyl (e.g., 2 to 8 membered heteroalkyl, 2 to 6 membered heteroalkyl, or 2 to 4 membered heteroalkyl). In embodiments, R³⁵ is an unsubstituted heteroalkyl (e.g., 2 to 8 membered heteroalkyl, 2 to 6 membered heteroalkyl, or 2 to 4 membered heteroalkyl). In embodiments, R³⁵ is R³⁶-substituted or unsubstituted cycloalkyl (e.g., C₃-C₈ cycloalkyl, C₃-C₆ cycloalkyl, or C₅-C₆ cycloalkyl). In embodiments, R³⁵ is R³⁶-substituted cycloalkyl (e.g., C₃-C₈ cycloalkyl, C₃-C₆ cycloalkyl, or C₅-C₆ cycloalkyl). In embodiments, R³⁵ is an unsubstituted cycloalkyl (e.g., C₃-C₈ cycloalkyl, C₃-C₆ cycloalkyl, or C₅-C₆ cycloalkyl). In embodiments, R³⁵ is R³⁶-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³⁵ is R³⁶-substituted heterocycloalkyl (e.g., 3 to 8 membered heterocycloalkyl, 3 to 6 membered heterocycloalkyl, or 5 to 6 membered heterocycloalkyl). In embodiments, R³⁵ is an unsubstituted heterocycloalkyl (e.g., 3 to 8 membered heterocycloalkyl, 3 to 6 membered heterocycloalkyl, or 5 to 6 membered heterocycloalkyl). In embodiments, R³⁵ is R³⁶-substituted or unsubstituted aryl (e.g., C₆-C₁₀ aryl, C₁₀ aryl, or phenyl). In embodiments, R³⁵ is R³⁶-substituted aryl (e.g., C₆-C₁₀ aryl, C₁₀ aryl, or phenyl). In embodiments, R³⁵ is an unsubstituted aryl (e.g., C₆-C₁₀ aryl, C₁₀ aryl, or phenyl). In embodiments, R³⁵ is R³⁶-substituted or unsubstituted heteroaryl (e.g., 5 to 10 membered heteroaryl, 5 to 9 membered heteroaryl, or 5 to 6 membered heteroaryl). In embodiments, R³⁵ is R³⁶-substituted heteroaryl (e.g., 5 to 10 membered heteroaryl, 5 to 9 membered heteroaryl, or 5 to 6 membered heteroaryl). In embodiments, R³⁵ is an unsubstituted heteroaryl (e.g., 5 to 10 membered heteroaryl, 5 to 9 membered heteroaryl, or 5 to 6 membered heteroaryl).

R³⁶ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂F, —OCH₂Cl, —OCH₂Br, —CH₂I, R³⁷-substituted or unsubstituted alkyl (e.g., C₁-C₈ alkyl, C₁-C₆ alkyl, or C₁-C₄ alkyl), R³⁷-substituted or unsubstituted heteroalkyl (e.g., 2 to 8 membered heteroalkyl, 2 to 6 membered heteroalkyl, or 2 to 4 membered heteroalkyl), R³⁷-substituted or unsubstituted cycloalkyl (e.g., C₃-C₈ cycloalkyl, C₃-C₆ cycloalkyl, or C₅-C₆ cycloalkyl), R³⁷-substituted or unsubstituted heterocycloalkyl (e.g., 3 to 8 membered heterocycloalkyl, 3 to 6 membered heterocycloalkyl, or 5 to 6 membered heterocycloalkyl), R³⁷-substituted or unsubstituted aryl (e.g., C₆-C₁₀ aryl, C₁₀ aryl, or phenyl), or R³⁷-substituted or unsubstituted heteroaryl (e.g., 5 to 10 membered heteroaryl, 5 to 9 membered heteroaryl, or 5 to 6 membered heteroaryl).

In embodiments, R³⁶ is R³⁷-substituted or unsubstituted alkyl (e.g., C₁-C₈ alkyl, C₁-C₆ alkyl, or C₁-C₄ alkyl). In embodiments, R³⁶ is R³⁷-substituted alkyl (e.g., C₁-C₈ alkyl, C₁-C₆ alkyl, or C₁-C₄ alkyl). In embodiments, R³⁶ is an unsubstituted alkyl (e.g., C₁-C₈ alkyl, C₁-C₆ alkyl, or C₁-C₄ alkyl). In embodiments, R³⁶ is R³⁷-substituted or unsubstituted heteroalkyl (e.g., 2 to 8 membered heteroalkyl, 2 to 6 membered heteroalkyl, or 2 to 4 membered heteroalkyl). In embodiments, R³⁶ is R³⁷-substituted heteroalkyl (e.g., 2 to 8 membered heteroalkyl, 2 to 6 membered heteroalkyl, or 2 to 4 membered heteroalkyl). In embodiments, R³⁶ is an unsubstituted heteroalkyl (e.g., 2 to 8 membered heteroalkyl, 2 to 6 membered heteroalkyl, or 2 to 4 membered heteroalkyl). In embodiments, R³⁶ is R³⁷-substituted or unsubstituted cycloalkyl (e.g., C₃-C₈ cycloalkyl, C₃-C₆ cycloalkyl, or C₅-C₆ cycloalkyl). In embodiments, R³⁶ is R³⁷-substituted cycloalkyl (e.g., C₃-C₈ cycloalkyl, C₃-C₆ cycloalkyl, or C₅-C₆ cycloalkyl). In embodiments, R³⁶ is an unsubstituted cycloalkyl (e.g., C₃-C₈ cycloalkyl, C₃-C₆ cycloalkyl, or C₅-C₆ cycloalkyl). In embodiments, R³⁶ is R³⁷-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³⁶ is R³⁷-substituted heterocycloalkyl (e.g., 3 to 8 membered heterocycloalkyl, 3 to 6 membered heterocycloalkyl, or 5 to 6 membered heterocycloalkyl). In embodiments, R³⁶ is an unsubstituted heterocycloalkyl (e.g., 3 to 8 membered heterocycloalkyl, 3 to 6 membered heterocycloalkyl, or 5 to 6 membered heterocycloalkyl). In embodiments, R³⁶ is R³⁷-substituted or unsubstituted aryl (e.g., C₆-C₁₀ aryl, C₁₀ aryl, or phenyl). In embodiments, R³⁶ is R³⁷-substituted aryl (e.g., C₆-C₁₀ aryl, C₁₀ aryl, or phenyl). In embodiments, R³⁶ is an unsubstituted aryl (e.g., C₆-C₁₀ aryl, C₁₀ aryl, or phenyl). In embodiments, R³⁶ is R³⁷-substituted or unsubstituted heteroaryl (e.g., 5 to 10 membered heteroaryl, 5 to 9 membered heteroaryl, or 5 to 6 membered heteroaryl). In embodiments, R³⁶ is R³⁷-substituted heteroaryl (e.g., 5 to 10 membered heteroaryl, 5 to 9 membered heteroaryl, or 5 to 6 membered heteroaryl). In embodiments, R³⁶ is an unsubstituted heteroaryl (e.g., 5 to 10 membered heteroaryl, 5 to 9 membered heteroaryl, or 5 to 6 membered heteroaryl).

In embodiments, R¹ is halogen, —CN, substituted or unsubstituted alkyl or substituted or unsubstituted heteroalkyl.

In embodiments, R^{1A} is hydrogen, halogen, —CF₃, —CCl₃, —CBr₃, —CI₃, —COOH, —CONH₂, R³⁵-substituted or unsubstituted alkyl (e.g., C₁-C₈ alkyl, C₁-C₆ alkyl, or C₁-C₄ alkyl), R³⁵-substituted or unsubstituted heteroalkyl (e.g., 2 to 8 membered heteroalkyl, 2 to 6 membered heteroalkyl, or 2 to 4 membered heteroalkyl), R³⁵-substituted or unsubstituted cycloalkyl (e.g., C₃-C₈ cycloalkyl, C₃-C₆ cycloalkyl, or C₅-C₆ cycloalkyl), R³⁵-substituted or unsubstituted heterocycloalkyl (e.g., 3 to 8 membered heterocycloalkyl, 3 to 6 membered heterocycloalkyl, or 5 to 6 membered heterocycloalkyl), R³⁵-substituted or unsubstituted aryl (e.g., C₆-C₁₀ aryl, C₁₀ aryl, or phenyl), or R³⁵-substituted or unsubstituted heteroaryl (e.g., 5 to 10 membered heteroaryl, 5 to 9 membered heteroaryl, or 5 to 6 membered heteroaryl).

In embodiments, R^{1A} is R³⁵-substituted or unsubstituted alkyl (e.g., C₁-C₈ alkyl, C₁-C₆ alkyl, or C₁-C₄ alkyl). In embodiments, R^{1A} is R³⁵-substituted alkyl (e.g., C₁-C₈ alkyl, C₁-C₆ alkyl, or C₁-C₄ alkyl). In embodiments, R^{1A} is an unsubstituted alkyl (e.g., C₁-C₈ alkyl, C₁-C₆ alkyl, or C₁-C₄ alkyl). In embodiments, R^{1A} is R³⁵-substituted or unsubstituted heteroalkyl (e.g., 2 to 8 membered heteroalkyl, 2 to 6 membered heteroalkyl, or 2 to 4 membered heteroalkyl). In embodiments, R^{1A} is R³⁵-substituted heteroalkyl (e.g., 2 to 8 membered heteroalkyl, 2 to 6 membered heteroalkyl, or 2 to 4 membered heteroalkyl). In embodiments, R^{1A} is an unsubstituted heteroalkyl (e.g., 2 to 8 membered heteroalkyl, 2 to 6 membered heteroalkyl, or 2 to 4 membered heteroalkyl). In embodiments, R^{1A} is R³⁵-substituted or unsubstituted cycloalkyl (e.g., C₃-C₈ cycloalkyl, C₃-C₆ cycloalkyl, or C₅-C₆ cycloalkyl). In embodiments, R^{1A} is R³⁵-substituted cycloalkyl (e.g., C₃-C₈ cycloalkyl, C₃-C₆ cycloalkyl, or C₅-C₆ cycloalkyl). In embodiments, R^{1A} is an unsubstituted cycloalkyl (e.g., C₃-C₈ cycloalkyl, C₃-C₆ cycloalkyl, or C₅-C₆ cycloalkyl). In embodiments, R^{1A} is R³⁵-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{1A} is R³⁵-substituted heterocycloalkyl (e.g., 3 to 8 membered heterocycloalkyl, 3 to 6 membered heterocycloalkyl, or 5 to 6 membered heterocycloalkyl). In embodiments, R^{1A} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{1A} is R³⁵-substituted or unsubstituted aryl (e.g., C₆-C₁₀ aryl, C₁₀ aryl, or phenyl). In embodiments, R^{1A} is R³⁵-substituted aryl (e.g., C₆-C₁₀ aryl, C₁₀ aryl, or phenyl). In embodiments, R^{1A} is an unsubstituted aryl (e.g., C₆-C₁₀ aryl, C₁₀ aryl, or phenyl). In embodiments, R^{1A} is R³⁵-substituted or unsubstituted heteroaryl (e.g., 5 to 10 membered heteroaryl, 5 to 9 membered heteroaryl, or 5 to 6 membered heteroaryl). In embodiments, R^{1A} is R³⁵-substituted heteroaryl (e.g., 5 to 10 membered heteroaryl, 5 to 9 membered heteroaryl, or 5 to 6 membered heteroaryl). In embodiments, R^{1A} is an unsubstituted heteroaryl (e.g., 5 to 10 membered heteroaryl, 5 to 9 membered heteroaryl, or 5 to 6 membered heteroaryl).

In embodiments, R^{1B} is hydrogen, halogen, —CF₃, —CCl₃, —CBr₃, —CI₃, —COOH, —CONH₂, R³⁵-substituted or unsubstituted alkyl (e.g., C₁-C₈ alkyl, C₁-C₆ alkyl, or C₁-C₄ alkyl), R³⁵-substituted or unsubstituted heteroalkyl (e.g., 2 to 8 membered heteroalkyl, 2 to 6 membered heteroalkyl, or 2 to 4 membered heteroalkyl), R³⁵-substituted or unsubstituted cycloalkyl (e.g., C₃-C₈ cycloalkyl, C₃-C₆ cycloalkyl, or C₅-C₆ cycloalkyl), R³⁵-substituted or unsubstituted heterocycloalkyl (e.g., 3 to 8 membered heterocycloalkyl, 3 to 6 membered heterocycloalkyl, or 5 to 6 membered heterocycloalkyl), R³⁵-substituted or unsubstituted aryl (e.g., C₆-C₁₀ aryl, C₁₀ aryl, or phenyl), or R³⁵-substituted or unsubstituted heteroaryl (e.g., 5 to 10 membered heteroaryl, 5 to 9 membered heteroaryl, or 5 to 6 membered heteroaryl). In embodiments, R^{1B} and R^{1C} substituents bonded to the same nitrogen atom may optionally be joined to form a R³⁵-substituted or unsubstituted heterocycloalkyl (e.g., 3 to 8 membered heterocycloalkyl, 3 to 6 membered heterocycloalkyl, or 5 to 6 membered heterocycloalkyl) or R³⁵-substituted or unsubstituted heteroaryl (e.g., 5 to 10 membered heteroaryl, 5 to 9 membered heteroaryl, or 5 to 6 membered heteroaryl).

In embodiments, R^{1B} is R³⁵-substituted or unsubstituted alkyl (e.g., C₁-C₈ alkyl, C₁-C₆ alkyl, or C₁-C₄ alkyl). In embodiments, R^{1B} is R³⁵-substituted alkyl (e.g., C₁-C₈ alkyl, C₁-C₆ alkyl, or C₁-C₄ alkyl). In embodiments, R^{1B} is an unsubstituted alkyl (e.g., C₁-C₈ alkyl, C₁-C₆ alkyl, or C₁-C₄ alkyl). In embodiments, R^{1B} is R³⁵-substituted or unsubstituted heteroalkyl (e.g., 2 to 8 membered heteroalkyl, 2 to 6 membered heteroalkyl, or 2 to 4 membered heteroalkyl). In embodiments, R^{1B} is R³⁵-substituted heteroalkyl (e.g., 2 to 8 membered heteroalkyl, 2 to 6 membered heteroalkyl, or 2 to 4 membered heteroalkyl). In embodiments, R^{1B} is an unsubstituted heteroalkyl (e.g., 2 to 8 membered heteroalkyl, 2 to 6 membered heteroalkyl, or 2 to 4 membered heteroalkyl). In embodiments, R^{1B} is R³⁵-substituted or unsubstituted cycloalkyl (e.g., C₃-C₈ cycloalkyl, C₃-C₆ cycloalkyl, or C₅-C₆ cycloalkyl). In embodiments, R^{1B} is R³⁵-substituted cycloalkyl (e.g., C₃-C₈ cycloalkyl, C₃-C₆ cycloalkyl, or C₅-C₆ cycloalkyl). In embodiments, R^{1B} is an unsubstituted cycloalkyl (e.g., C₃-C₈ cycloalkyl, C₃-C₆ cycloalkyl, or C₅-C₆ cycloalkyl). In embodiments, R^{1B} is R³⁵-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{1B} is R³⁵-substituted heterocycloalkyl (e.g., 3 to 8 membered heterocycloalkyl, 3 to 6 membered heterocycloalkyl, or 5 to 6 membered heterocycloalkyl). In embodiments, R^{1B} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{1B} is R³⁵-substituted or unsubstituted aryl (e.g., C₆-C₁₀ aryl, C₁₀ aryl, or phenyl). In embodiments, R^{1B} is R³⁵-substituted aryl (e.g., C₆-C₁₀ aryl, C₁₀ aryl, or phenyl). In embodiments, R^{1B} is an unsubstituted aryl (e.g., C₆-C₁₀ aryl, C₁₀ aryl, or phenyl). In embodiments, R^{1B} is R³⁵-substituted or unsubstituted heteroaryl (e.g., 5 to 10 membered heteroaryl, 5 to 9 membered heteroaryl, or 5 to 6 membered heteroaryl). In embodiments, R^{1B} is R³⁵-substituted heteroaryl (e.g., 5 to 10 membered heteroaryl, 5 to 9 membered heteroaryl, or 5 to 6 membered heteroaryl). In embodiments, R^{1B} is an unsubstituted heteroaryl (e.g., 5 to 10 membered heteroaryl, 5 to 9 membered heteroaryl, or 5 to 6 membered heteroaryl).

In embodiments, R^{1C} is hydrogen, halogen, —CF₃, —CCl₃, —CBr₃, —CI₃, —COOH, —CONH₂, R³⁵-substituted or unsubstituted alkyl (e.g., C₁-C₈ alkyl, C₁-C₆ alkyl, or C₁-C₄ alkyl), R³⁵-substituted or unsubstituted heteroalkyl (e.g., 2 to 8 membered heteroalkyl, 2 to 6 membered heteroalkyl, or 2 to 4 membered heteroalkyl), R³⁵-substituted or unsubstituted cycloalkyl (e.g., C₃-C₈ cycloalkyl, C₃-C₆ cycloalkyl, or C₅-C₆ cycloalkyl), R³⁵-substituted or unsubstituted heterocycloalkyl (e.g., 3 to 8 membered heterocycloalkyl, 3 to 6 membered heterocycloalkyl, or 5 to 6 membered heterocycloalkyl), R³⁵-substituted or unsubstituted aryl (e.g., C₆-C₁₀ aryl, C₁₀ aryl, or phenyl), or R³⁵-substituted or unsubstituted heteroaryl (e.g., 5 to 10 membered heteroaryl, 5 to 9 membered heteroaryl, or 5 to 6 membered heteroaryl). In embodiments, R^{1B} and R^{1C} substituents bonded to the same nitrogen atom may optionally be joined to form a R³⁵⁻substituted or unsubstituted heterocycloalkyl (e.g., 3 to 8 membered heterocycloalkyl, 3 to 6 membered heterocycloalkyl, or 5 to 6 membered heterocycloalkyl) or R³⁵-substituted or unsubstituted heteroaryl (e.g., 5 to 10 membered heteroaryl, 5 to 9 membered heteroaryl, or 5 to 6 membered heteroaryl).

In embodiments, R^{1C} is R³⁵-substituted or unsubstituted alkyl (e.g., C₁-C₈ alkyl, C₁-C₆ alkyl, or C₁-C₄ alkyl). In embodiments, R^{1C} is R³⁵-substituted alkyl (e.g., C₁-C₈ alkyl, C₁-C₆ alkyl, or C₁-C₄ alkyl). In embodiments, R^{1C} is an unsubstituted alkyl (e.g., C₁-C₈ alkyl, C₁-C₆ alkyl, or C₁-C₄ alkyl). In embodiments, R^{1C} is R³⁵-substituted or unsubstituted heteroalkyl (e.g., 2 to 8 membered heteroalkyl, 2 to 6 membered heteroalkyl, or 2 to 4 membered heteroalkyl). In embodiments, R^{1C} is R³⁵-substituted heteroalkyl (e.g., 2 to 8 membered heteroalkyl, 2 to 6 membered heteroalkyl, or 2 to 4 membered heteroalkyl). In embodiments, R^{1C} is an unsubstituted heteroalkyl (e.g., 2 to 8 membered heteroalkyl, 2 to 6 membered heteroalkyl, or 2 to 4 membered heteroalkyl). In embodiments, R^{1C} is R³⁵-substituted or unsubstituted cycloalkyl (e.g., C₃-C₈ cycloalkyl, C₃-C₆ cycloalkyl, or C₅-C₆ cycloalkyl). In embodiments, R^{1C} is R³⁵-substituted cycloalkyl (e.g., C₃-C₈ cycloalkyl, C₃-C₆ cycloalkyl, or C₅-C₆ cycloalkyl). In embodiments, R^{1C} is an unsubstituted cycloalkyl (e.g., C₃-C₈ cycloalkyl, C₃-C₆ cycloalkyl, or C₅-C₆ cycloalkyl). In embodiments, R^{1C} is R³⁵-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{1C} is R³⁵-substituted heterocycloalkyl (e.g., 3 to 8 membered heterocycloalkyl, 3 to 6 membered heterocycloalkyl, or 5 to 6 membered heterocycloalkyl). In embodiments, R^{1C} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{1C} is R³⁵-substituted or unsubstituted aryl (e.g., C₆-C₁₀ aryl, C₁₀ aryl, or phenyl). In embodiments, R^{1C} is R³⁵-substituted aryl (e.g., C₆-C₁₀ aryl, C₁₀ aryl, or phenyl). In embodiments, R^{1C} is an unsubstituted aryl (e.g., C₆-C₁₀ aryl, C₁₀ aryl, or phenyl). In embodiments, R^{1C} is R³⁵-substituted or unsubstituted heteroaryl (e.g., 5 to 10 membered heteroaryl, 5 to 9 membered heteroaryl, or 5 to 6 membered heteroaryl). In embodiments, R^{1C} is R³⁵-substituted heteroaryl (e.g., 5 to 10 membered heteroaryl, 5 to 9 membered heteroaryl, or 5 to 6 membered heteroaryl). In embodiments, R^{1C} is an unsubstituted heteroaryl (e.g., 5 to 10 membered heteroaryl, 5 to 9 membered heteroaryl, or 5 to 6 membered heteroaryl).

In embodiments, R^{1D} is hydrogen, halogen, —CF₃, —CCl₃, —CBr₃, —CI₃, —COOH, —CONH₂, R³⁵-substituted or unsubstituted alkyl (e.g., C₁-C₈ alkyl, C₁-C₆ alkyl, or C₁-C₄ alkyl), R³⁵-substituted or unsubstituted heteroalkyl (e.g., 2 to 8 membered heteroalkyl, 2 to 6 membered heteroalkyl, or 2 to 4 membered heteroalkyl), R³⁵-substituted or unsubstituted cycloalkyl (e.g., C₃-C₈ cycloalkyl, C₃-C₆ cycloalkyl, or C₅-C₆ cycloalkyl), R³⁵-substituted or unsubstituted heterocycloalkyl (e.g., 3 to 8 membered heterocycloalkyl, 3 to 6 membered heterocycloalkyl, or 5 to 6 membered heterocycloalkyl), R³⁵-substituted or unsubstituted aryl (e.g., C₆-C₁₀ aryl, C₁₀ aryl, or phenyl), or R³⁵-substituted or unsubstituted heteroaryl (e.g., 5 to 10 membered heteroaryl, 5 to 9 membered heteroaryl, or 5 to 6 membered heteroaryl).

In embodiments, R^{1D} is R³⁵-substituted or unsubstituted alkyl (e.g., C₁-C₈ alkyl, C₁-C₆ alkyl, or C₁-C₄ alkyl). In embodiments, R^{1D} is R³⁵-substituted alkyl (e.g., C₁-C₈ alkyl, C₁-C₆ alkyl, or C₁-C₄ alkyl). In embodiments, R^{1D} is an unsubstituted alkyl (e.g., C₁-C₈ alkyl, C₁-C₆ alkyl, or C₁-C₄ alkyl). In embodiments, R^{1D} is R³⁵-substituted or unsubstituted heteroalkyl (e.g., 2 to 8 membered heteroalkyl, 2 to 6 membered heteroalkyl, or 2 to 4 membered heteroalkyl). In embodiments, R^{1D} is R³⁵-substituted heteroalkyl (e.g., 2 to 8 membered heteroalkyl, 2 to 6 membered heteroalkyl, or 2 to 4 membered heteroalkyl). In embodiments, R^{1D} is an unsubstituted heteroalkyl (e.g., 2 to 8 membered heteroalkyl, 2 to 6 membered heteroalkyl, or 2 to 4 membered heteroalkyl). In embodiments, R^{1D} is R³⁵-substituted or unsubstituted cycloalkyl (e.g., C₃-C₈ cycloalkyl, C₃-C₆ cycloalkyl, or C₅-C₆ cycloalkyl). In embodiments, R^{1D} is R³⁵-substituted cycloalkyl (e.g., C₃-C₈ cycloalkyl, C₃-C₆ cycloalkyl, or C₅-C₆ cycloalkyl). In embodiments, R^{1D} is an unsubstituted cycloalkyl (e.g., C₃-C₈ cycloalkyl, C₃-C₆ cycloalkyl, or C₅-C₆ cycloalkyl). In embodiments, R^{1D} is R³⁵-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{1D} is R³⁵-substituted heterocycloalkyl (e.g., 3 to 8 membered heterocycloalkyl, 3 to 6 membered heterocycloalkyl, or 5 to 6 membered heterocycloalkyl). In embodiments, R^{1D} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{1D} is R³⁵-substituted or unsubstituted aryl (e.g., C₆-C₁₀ aryl, C₁₀ aryl, or phenyl). In embodiments, R^{1D} is R³⁵-substituted aryl (e.g., C₆-C₁₀ aryl, C₁₀ aryl, or phenyl). In embodiments, R^{1D} is an unsubstituted aryl (e.g., C₆-C₁₀ aryl, C₁₀ aryl, or phenyl). In embodiments, R^{1D} is R³⁵-substituted or unsubstituted heteroaryl (e.g., 5 to 10 membered heteroaryl, 5 to 9 membered heteroaryl, or 5 to 6 membered heteroaryl). In embodiments, R^{1D} is R³⁵-substituted heteroaryl (e.g., 5 to 10 membered heteroaryl, 5 to 9 membered heteroaryl, or 5 to 6 membered heteroaryl). In embodiments, R^{1D} is an unsubstituted heteroaryl (e.g., 5 to 10 membered heteroaryl, 5 to 9 membered heteroaryl, or 5 to 6 membered heteroaryl)

In embodiments, R^{1.1}, R^{1.2}, R^{1.3} and R^{1.4} are independently hydrogen, halogen, —CN, substituted or unsubstituted alkyl or substituted or unsubstituted heteroalkyl. In embodiments, R^{1.1} is hydrogen, halogen, —CN, substituted or unsubstituted alkyl or substituted or unsubstituted heteroalkyl. In embodiments, R^{1.2} is hydrogen, halogen, —CN, substituted or unsubstituted alkyl or substituted or unsubstituted heteroalkyl. In embodiments, R^{1.3} is hydrogen, halogen, —CN, substituted or unsubstituted alkyl or substituted or unsubstituted heteroalkyl. In embodiments, R^{1.4} is hydrogen, halogen, —CN, substituted or unsubstituted alkyl or substituted or unsubstituted heteroalkyl. In embodiments, R^{1.1} is hydrogen. In embodiments, R^{1.4} is —CH₂NH₂.

In embodiments, at least two of R^{1.1}, R^{1.2}, R^{1.3} and R^{1.4} are not hydrogen. In embodiments, R^{1.1} is not hydrogen. In embodiments, R^{1.2} is not hydrogen. In embodiments, R^{1.3} is not hydrogen. In embodiments, R^{1.4} is not hydrogen.

In embodiments, R^{1.1} and R^{1.2} are joined to form a substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂H, —SOᵥ₂NH₂, —NHNH₂, —ONH₂, —NHC(O)NHNH₂, —NHC(O)NH₂, —N(O)ₘ₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R² is a substituted or unsubstituted alkyl (e.g., C₁-C₈ alkyl, C₁-C₆ alkyl, or C₁-C₄ alkyl). In embodiments, R² is a substituted alkyl (e.g., C₁-C₈ alkyl, C₁-C₆ alkyl, or C₁-C₄ alkyl). In embodiments, R² is an unsubstituted alkyl (e.g., C₁-C₈ alkyl, C₁-C₆ alkyl, or C₁-C₄ alkyl). In embodiments, R² is a substituted or unsubstituted heteroalkyl (e.g., 2 to 8 membered heteroalkyl, 2 to 6 membered heteroalkyl, or 2 to 4 membered heteroalkyl). In embodiments, R² is a substituted heteroalkyl (e.g., 2 to 8 membered heteroalkyl, 2 to 6 membered heteroalkyl, or 2 to 4 membered heteroalkyl). In embodiments, R² is an unsubstituted heteroalkyl (e.g., 2 to 8 membered heteroalkyl, 2 to 6 membered heteroalkyl, or 2 to 4 membered heteroalkyl). In embodiments, R² is a substituted or unsubstituted cycloalkyl (e.g., C₃-C₈ cycloalkyl, C₃-C₆ cycloalkyl, or C₅-C₆ cycloalkyl). In embodiments, R² is a substituted cycloalkyl (e.g., C₃-C₈ cycloalkyl, C₃-C₆ cycloalkyl, or C₅-C₆ cycloalkyl). In embodiments, R² is an unsubstituted cycloalkyl (e.g., C₃-C₈ cycloalkyl, C₃-C₆ cycloalkyl, or C₅-C₆ cycloalkyl). In embodiments, R² is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R² is a substituted heterocycloalkyl (e.g., 3 to 8 membered heterocycloalkyl, 3 to 6 membered heterocycloalkyl, or 5 to 6 membered heterocycloalkyl). In embodiments, R² is an unsubstituted heterocycloalkyl (e.g., 3 to 8 membered heterocycloalkyl, 3 to 6 membered heterocycloalkyl, or 5 to 6 membered heterocycloalkyl). In embodiments, R² is a substituted or unsubstituted aryl (e.g., C₆-C₁₀ aryl, C₁₀ aryl, or phenyl). In embodiments, R² is a substituted aryl (e.g., C₆-C₁₀ aryl, C₁₀ aryl, or phenyl). In embodiments, R² is an unsubstituted aryl (e.g., C₆-C₁₀ aryl, C₁₀ aryl, or phenyl). In embodiments, R² is a substituted or unsubstituted heteroaryl (e.g., 5 to 10 membered heteroaryl, 5 to 9 membered heteroaryl, or 5 to 6 membered heteroaryl). In embodiments, R² is a substituted heteroaryl (e.g., 5 to 10 membered heteroaryl, 5 to 9 membered heteroaryl, or 5 to 6 membered heteroaryl). In embodiments, R² is an unsubstituted heteroaryl (e.g., 5 to 10 membered heteroaryl, 5 to 9 membered heteroaryl, or 5 to 6 membered heteroaryl).

In embodiments, R² is hydrogen, halogen, substituted or unsubstituted alkyl or substituted or unsubstituted heteroalkyl. In embodiments, R² is hydrogen or halogen. In embodiments, R² is hydrogen.

In embodiments, R² and R³ are independently hydrogen, halogen, substituted or unsubstituted alkyl or substituted or unsubstituted heteroalkyl. In embodiments, R² and R³ are unsubstituted methyl.

In embodiments, R³ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃H, —SOᵥ₃NH₂, —NHNH₂, —ONH₂, —NHC(O)NHNH₂, —NHC(O)NH₂, —N(O)ₘ₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, R³ is hydrogen.

In embodiments, R³ is a substituted or unsubstituted alkyl (e.g., C₁-C₈ alkyl, C₁-C₆ alkyl, or C₁-C₄ alkyl). In embodiments, R³ is a substituted alkyl (e.g., C₁-C₈ alkyl, C₁-C₆ alkyl, or C₁-C₄ alkyl). In embodiments, R³ is an unsubstituted alkyl (e.g., C₁-C₈ alkyl, C₁-C₆ alkyl, or C₁-C₄ alkyl). In embodiments, R³ is a substituted or unsubstituted heteroalkyl (e.g., 2 to 8 membered heteroalkyl, 2 to 6 membered heteroalkyl, or 2 to 4 membered heteroalkyl). In embodiments, R³ is a substituted heteroalkyl (e.g., 2 to 8 membered heteroalkyl, 2 to 6 membered heteroalkyl, or 2 to 4 membered heteroalkyl). In embodiments, R³ is an unsubstituted heteroalkyl (e.g., 2 to 8 membered heteroalkyl, 2 to 6 membered heteroalkyl, or 2 to 4 membered heteroalkyl). In embodiments, R³ is a substituted or unsubstituted cycloalkyl (e.g., C₃-C₈ cycloalkyl, C₃-C₆ cycloalkyl, or C₅-C₆ cycloalkyl). In embodiments, R³ is a substituted cycloalkyl (e.g., C₃-C₈ cycloalkyl, C₃-C₆ cycloalkyl, or C₅-C₆ cycloalkyl). In embodiments, R³ is an unsubstituted cycloalkyl (e.g., C₃-C₈ cycloalkyl, C₃-C₆ cycloalkyl, or C₅-C₆ cycloalkyl). In embodiments, R³ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³ is a substituted heterocycloalkyl (e.g., 3 to 8 membered heterocycloalkyl, 3 to 6 membered heterocycloalkyl, or 5 to 6 membered heterocycloalkyl). In embodiments, R³ is an unsubstituted heterocycloalkyl (e.g., 3 to 8 membered heterocycloalkyl, 3 to 6 membered heterocycloalkyl, or 5 to 6 membered heterocycloalkyl). In embodiments, R³ is a substituted or unsubstituted aryl (e.g., C₆-C₁₀ aryl, C₁₀ aryl, or phenyl). In embodiments, R³ is a substituted aryl (e.g., C₆-C₁₀ aryl, C₁₀ aryl, or phenyl). In embodiments, R³ is an unsubstituted aryl (e.g., C₆-C₁₀ aryl, C₁₀ aryl, or phenyl). In embodiments, R³ is a substituted or unsubstituted heteroaryl (e.g., 5 to 10 membered heteroaryl, 5 to 9 membered heteroaryl, or 5 to 6 membered heteroaryl). In embodiments, R³ is a substituted heteroaryl (e.g., 5 to 10 membered heteroaryl, 5 to 9 membered heteroaryl, or 5 to 6 membered heteroaryl). In embodiments, R³ is an unsubstituted heteroaryl (e.g., 5 to 10 membered heteroaryl, 5 to 9 membered heteroaryl, or 5 to 6 membered heteroaryl).

In embodiments, R³ is hydrogen, halogen, substituted or unsubstituted alkyl or substituted or unsubstituted heteroalkyl. In embodiments, R³ is halogen, unsubstituted methyl or —CF₃. In embodiments, R³ is hydrogen or halogen. In embodiments, R³ is halogen, unsubstituted methyl or —CF₃.

In embodiments, R⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₄H, —SOᵥ₄NH₂, —NHNH₂, —ONH₂, —NHC(O)NHNH₂, —NHC(O)NH₂, —N(O)ₘ₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, R⁴ is hydrogen.

In embodiments, R⁴ is a substituted or unsubstituted alkyl (e.g., C₁-C₈ alkyl, C₁-C₆ alkyl, or C₁-C₄ alkyl). In embodiments, R⁴ is a substituted alkyl (e.g., C₁-C₈ alkyl, C₁-C₆ alkyl, or C₁-C₄ alkyl). In embodiments, R⁴ is an unsubstituted alkyl (e.g., C₁-C₈ alkyl, C₁-C₆ alkyl, or C₁-C₄ alkyl). In embodiments, R⁴ is a substituted or unsubstituted heteroalkyl (e.g., 2 to 8 membered heteroalkyl, 2 to 6 membered heteroalkyl, or 2 to 4 membered heteroalkyl). In embodiments, R⁴ is a substituted heteroalkyl (e.g., 2 to 8 membered heteroalkyl, 2 to 6 membered heteroalkyl, or 2 to 4 membered heteroalkyl). In embodiments, R⁴ is an unsubstituted heteroalkyl (e.g., 2 to 8 membered heteroalkyl, 2 to 6 membered heteroalkyl, or 2 to 4 membered heteroalkyl). In embodiments, R⁴ is a substituted or unsubstituted cycloalkyl (e.g., C₃-C₈ cycloalkyl, C₃-C₆ cycloalkyl, or C₅-C₆ cycloalkyl). In embodiments, R⁴ is a substituted cycloalkyl (e.g., C₃-C₈ cycloalkyl, C₃-C₆ cycloalkyl, or C₅-C₆ cycloalkyl). In embodiments, R⁴ is an unsubstituted cycloalkyl (e.g., C₃-C₈ cycloalkyl, C₃-C₆ cycloalkyl, or C₅-C₆ cycloalkyl). In embodiments, R⁴ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴ is a substituted heterocycloalkyl (e.g., 3 to 8 membered heterocycloalkyl, 3 to 6 membered heterocycloalkyl, or 5 to 6 membered heterocycloalkyl). In embodiments, R⁴ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁴ is a substituted or unsubstituted aryl (e.g., C₆-C₁₀ aryl, C₁₀ aryl, or phenyl). In embodiments, R⁴ is a substituted aryl (e.g., C₆-C₁₀ aryl, C₁₀ aryl, or phenyl). In embodiments, R⁴ is an unsubstituted aryl (e.g., C₆-C₁₀ aryl, C₁₀ aryl, or phenyl). In embodiments, R⁴ is a substituted or unsubstituted heteroaryl (e.g., 5 to 10 membered heteroaryl, 5 to 9 membered heteroaryl, or 5 to 6 membered heteroaryl). In embodiments, R⁴ is a substituted heteroaryl (e.g., 5 to 10 membered heteroaryl, 5 to 9 membered heteroaryl, or 5 to 6 membered heteroaryl). In embodiments, R⁴ is an unsubstituted heteroaryl (e.g., 5 to 10 membered heteroaryl, 5 to 9 membered heteroaryl, or 5 to 6 membered heteroaryl).

In embodiments, R⁴ is hydrogen, substituted or unsubstituted alkyl or substituted or unsubstituted heteroalkyl. In embodiments, R⁴ is hydrogen or unsubstituted methyl.

In embodiments, R⁴ is unsubstituted methyl. In embodiments, R⁴ is substituted C₁-C₄ alkyl. In embodiments, R⁴ is an unsubstituted C₁-C₄ alkyl. In embodiments, R⁴ is an unsubstituted C₁-C₄ alkenyl. In embodiments, R⁴ is an unsubstituted C₃ alkenyl. In embodiments, R⁴ is

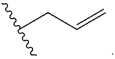 [SMILES: *CC=C]

In embodiments, R⁵ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R⁵ is halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R⁵ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R³⁸-substituted or unsubstituted alkyl (e.g., C₁-C₈ alkyl, C₁-C₆ alkyl, or C₁-C₄ alkyl), R³⁸-substituted or unsubstituted heteroalkyl (e.g., 2 to 8 membered heteroalkyl, 2 to 6 membered heteroalkyl, or 2 to 4 membered heteroalkyl), R³⁸-substituted or unsubstituted cycloalkyl (e.g., C₃-C₈ cycloalkyl, C₃-C₆ cycloalkyl, or C₅-C₆ cycloalkyl), R³⁸-substituted or unsubstituted heterocycloalkyl (e.g., 3 to 8 membered heterocycloalkyl, 3 to 6 membered heterocycloalkyl, or 5 to 6 membered heterocycloalkyl), R³⁸-substituted or unsubstituted aryl (e.g., C₆-C₁₀ aryl, C₁₀ aryl, or phenyl), or R³⁸-substituted or unsubstituted heteroaryl (e.g., 5 to 10 membered heteroaryl, 5 to 9 membered heteroaryl, or 5 to 6 membered heteroaryl).

In embodiments, R⁵ is R³⁸-substituted or unsubstituted alkyl (e.g., C₁-C₈ alkyl, C₁-C₆ alkyl, or C₁-C₄ alkyl). In embodiments, R⁵ is R³⁸-substituted alkyl (e.g., C₁-C₈ alkyl, C₁-C₆ alkyl, or C₁-C₄ alkyl). In embodiments, R⁵ is an unsubstituted alkyl (e.g., C₁-C₈ alkyl, C₁-C₆ alkyl, or C₁-C₄ alkyl). In embodiments, R⁵ is R³⁸-substituted or unsubstituted heteroalkyl (e.g., 2 to 8 membered heteroalkyl, 2 to 6 membered heteroalkyl, or 2 to 4 membered heteroalkyl). In embodiments, R⁵ is R³⁸-substituted heteroalkyl (e.g., 2 to 8 membered heteroalkyl, 2 to 6 membered heteroalkyl, or 2 to 4 membered heteroalkyl). In embodiments, R⁵ is an unsubstituted heteroalkyl (e.g., 2 to 8 membered heteroalkyl, 2 to 6 membered heteroalkyl, or 2 to 4 membered heteroalkyl). In embodiments, R⁵ is R³⁸-substituted or unsubstituted cycloalkyl (e.g., C₃-C₈ cycloalkyl, C₃-C₆ cycloalkyl, or C₅-C₆ cycloalkyl). In embodiments, R⁵ is R³⁸-substituted cycloalkyl (e.g., C₃-C₈ cycloalkyl, C₃-C₆ cycloalkyl, or C₅-C₆ cycloalkyl). In embodiments, R⁵ is an unsubstituted cycloalkyl (e.g., C₃-C₈ cycloalkyl, C₃-C₆ cycloalkyl, or C₅-C₆ cycloalkyl). In embodiments, R⁵ is R³⁸-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁵ is R³⁸-substituted heterocycloalkyl (e.g., 3 to 8 membered heterocycloalkyl, 3 to 6 membered heterocycloalkyl, or 5 to 6 membered heterocycloalkyl). In embodiments, R⁵ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁵ is R³⁸-substituted or unsubstituted aryl (e.g., C₆-C₁₀ aryl, C₁₀ aryl, or phenyl). In embodiments, R⁵ is R³⁸-substituted aryl (e.g., C₆-C₁₀ aryl, C₁₀ aryl, or phenyl). In embodiments, R⁵ is an unsubstituted aryl (e.g., C₆-C₁₀ aryl, C₁₀ aryl, or phenyl). In embodiments, R⁵ is R³⁸-substituted or unsubstituted heteroaryl (e.g., 5 to 10 membered heteroaryl, 5 to 9 membered heteroaryl, or 5 to 6 membered heteroaryl). In embodiments, R⁵ is R³⁸-substituted heteroaryl (e.g., 5 to 10 membered heteroaryl, 5 to 9 membered heteroaryl, or 5 to 6 membered heteroaryl). In embodiments, R⁵ is an unsubstituted heteroaryl (e.g., 5 to 10 membered heteroaryl, 5 to 9 membered heteroaryl, or 5 to 6 membered heteroaryl).

R³⁸ is independently oxo, halogen, —CCl₃, —CBr₃, —CF₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂F, —OCH₂Cl, —OCH₂Br, —OCH₂I, R³⁹-substituted or unsubstituted alkyl (e.g., C₁-C₈ alkyl, C₁-C₆ alkyl, or C₁-C₄ alkyl), R³⁹-substituted or unsubstituted heteroalkyl (e.g., 2 to 8 membered heteroalkyl, 2 to 6 membered heteroalkyl, or 2 to 4 membered heteroalkyl), R³⁹-substituted or unsubstituted cycloalkyl (e.g., C₃-C₈ cycloalkyl, C₃-C₆ cycloalkyl, or C₅-C₆ cycloalkyl), R³⁹-substituted or unsubstituted heterocycloalkyl (e.g., 3 to 8 membered heterocycloalkyl, 3 to 6 membered heterocycloalkyl, or 5 to 6 membered heterocycloalkyl), R³⁹-substituted or unsubstituted aryl (e.g., C₆-C₁₀ aryl, C₁₀ aryl, or phenyl), or R³⁹-substituted or unsubstituted heteroaryl (e.g., 5 to 10 membered heteroaryl, 5 to 9 membered heteroaryl, or 5 to 6 membered heteroaryl).

In embodiments, R³⁸ is R³⁹-substituted or unsubstituted alkyl (e.g., C₁-C₈ alkyl, C₁-C₆ alkyl, or C₁-C₄ alkyl). In embodiments, R³⁸ is R³⁹-substituted alkyl (e.g., C₁-C₈ alkyl, C₁-C₆ alkyl, or C₁-C₄ alkyl). In embodiments, R³⁸ is an unsubstituted alkyl (e.g., C₁-C₈ alkyl, C₁-C₆ alkyl, or C₁-C₄ alkyl). In embodiments, R³⁸ is R³⁹-substituted or unsubstituted heteroalkyl (e.g., 2 to 8 membered heteroalkyl, 2 to 6 membered heteroalkyl, or 2 to 4 membered heteroalkyl). In embodiments, R³⁸ is R³⁹-substituted heteroalkyl (e.g., 2 to 8 membered heteroalkyl, 2 to 6 membered heteroalkyl, or 2 to 4 membered heteroalkyl). In embodiments, R³⁸ is an unsubstituted heteroalkyl (e.g., 2 to 8 membered heteroalkyl, 2 to 6 membered heteroalkyl, or 2 to 4 membered heteroalkyl). In embodiments, R³⁸ is R³⁹-substituted or unsubstituted cycloalkyl (e.g., C₃-C₈ cycloalkyl, C₃-C₆ cycloalkyl, or C₅-C₆ cycloalkyl). In embodiments, R³⁸ is R³⁹-substituted cycloalkyl (e.g., C₃-C₈ cycloalkyl, C₃-C₆ cycloalkyl, or C₅-C₆ cycloalkyl). In embodiments, R³⁸ is an unsubstituted cycloalkyl (e.g., C₃-C₈ cycloalkyl, C₃-C₆ cycloalkyl, or C₅-C₆ cycloalkyl). In embodiments, R³⁸ is R³⁹-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³⁸ is R³⁹-substituted heterocycloalkyl (e.g., 3 to 8 membered heterocycloalkyl, 3 to 6 membered heterocycloalkyl, or 5 to 6 membered heterocycloalkyl). In embodiments, R³⁸ is an unsubstituted heterocycloalkyl (e.g., 3 to 8 membered heterocycloalkyl, 3 to 6 membered heterocycloalkyl, or 5 to 6 membered heterocycloalkyl). In embodiments, R³⁸ is R³⁹-substituted or unsubstituted aryl (e.g., C₆-C₁₀ aryl, C₁₀ aryl, or phenyl). In embodiments, R³⁸ is R³⁹-substituted aryl (e.g., C₆-C₁₀ aryl, C₁₀ aryl, or phenyl). In embodiments, R³⁸ is an unsubstituted aryl (e.g., C₆-C₁₀ aryl, C₁₀ aryl, or phenyl). In embodiments, R³⁸ is R³⁹-substituted or unsubstituted heteroaryl (e.g., 5 to 10 membered heteroaryl, 5 to 9 membered heteroaryl, or 5 to 6 membered heteroaryl). In embodiments, R³⁸ is R³⁹-substituted heteroaryl (e.g., 5 to 10 membered heteroaryl, 5 to 9 membered heteroaryl, or 5 to 6 membered heteroaryl). In embodiments, R³⁸ is an unsubstituted heteroaryl (e.g., 5 to 10 membered heteroaryl, 5 to 9 membered heteroaryl, or 5 to 6 membered heteroaryl).

R³⁹ is independently oxo, halogen, —CCl₃, —CBr₃, —CF₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂F, —OCH₂Cl, —OCH₂Br, —OCH₂I, R⁴⁰-substituted or unsubstituted alkyl (e.g., C₁-C₈ alkyl, C₁-C₆ alkyl, or C₁-C₄ alkyl), R⁴⁰-substituted or unsubstituted heteroalkyl (e.g., 2 to 8 membered heteroalkyl, 2 to 6 membered heteroalkyl, or 2 to 4 membered heteroalkyl), R⁴⁰-substituted or unsubstituted cycloalkyl (e.g., C₃-C₈ cycloalkyl, C₃-C₆ cycloalkyl, or C₅-C₆ cycloalkyl), R⁴⁰-substituted or unsubstituted heterocycloalkyl (e.g., 3 to 8 membered heterocycloalkyl, 3 to 6 membered heterocycloalkyl, or 5 to 6 membered heterocycloalkyl), R⁴⁰-substituted or unsubstituted aryl (e.g., C₆-C₁₀ aryl, C₁₀ aryl, or phenyl), or R⁴⁰-substituted or unsubstituted heteroaryl (e.g., 5 to 10 membered heteroaryl, 5 to 9 membered heteroaryl, or 5 to 6 membered heteroaryl).

In embodiments, R³⁹ is R⁴⁰-substituted or unsubstituted alkyl (e.g., C₁-C₈ alkyl, C₁-C₆ alkyl, or C₁-C₄ alkyl). In embodiments, R³⁹ is R⁴⁰-substituted alkyl (e.g., C₁-C₈ alkyl, C₁-C₆ alkyl, or C₁-C₄ alkyl). In embodiments, R³⁹ is an unsubstituted alkyl (e.g., C₁-C₈ alkyl, C₁-C₆ alkyl, or C₁-C₄ alkyl). In embodiments, R³⁹ is R⁴⁰-substituted or unsubstituted heteroalkyl (e.g., 2 to 8 membered heteroalkyl, 2 to 6 membered heteroalkyl, or 2 to 4 membered heteroalkyl). In embodiments, R³⁹ is R⁴⁰-substituted heteroalkyl (e.g., 2 to 8 membered heteroalkyl, 2 to 6 membered heteroalkyl, or 2 to 4 membered heteroalkyl). In embodiments, R³⁹ is an unsubstituted heteroalkyl (e.g., 2 to 8 membered heteroalkyl, 2 to 6 membered heteroalkyl, or 2 to 4 membered heteroalkyl). In embodiments, R³⁹ is R⁴⁰-substituted or unsubstituted cycloalkyl (e.g., C₃-C₈ cycloalkyl, C₃-C₆ cycloalkyl, or C₅-C₆ cycloalkyl). In embodiments, R³⁹ is R⁴⁰-substituted cycloalkyl (e.g., C₃-C₈ cycloalkyl, C₃-C₆ cycloalkyl, or C₅-C₆ cycloalkyl). In embodiments, R³⁹ is an unsubstituted cycloalkyl (e.g., C₃-C₈ cycloalkyl, C₃-C₆ cycloalkyl, or C₅-C₆ cycloalkyl). In embodiments, R³⁹ is R⁴⁰-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³⁹ is R⁴⁰-substituted heterocycloalkyl (e.g., 3 to 8 membered heterocycloalkyl, 3 to 6 membered heterocycloalkyl, or 5 to 6 membered heterocycloalkyl). In embodiments, R³⁹ is an unsubstituted heterocycloalkyl (e.g., 3 to 8 membered heterocycloalkyl, 3 to 6 membered heterocycloalkyl, or 5 to 6 membered heterocycloalkyl). In embodiments, R³⁹ is R⁴⁰-substituted or unsubstituted aryl (e.g., C₆-C₁₀ aryl, C₁₀ aryl, or phenyl). In embodiments, R³⁹ is R⁴⁰-substituted aryl (e.g., C₆-C₁₀ aryl, C₁₀ aryl, or phenyl). In embodiments, R³⁹ is an unsubstituted aryl (e.g., C₆-C₁₀ aryl, C₁₀ aryl, or phenyl). In embodiments, R³⁹ is R⁴⁰-substituted or unsubstituted heteroaryl (e.g., 5 to 10 membered heteroaryl, 5 to 9 membered heteroaryl, or 5 to 6 membered heteroaryl). In embodiments, R³⁹ is R⁴⁰-substituted heteroaryl (e.g., 5 to 10 membered heteroaryl, 5 to 9 membered heteroaryl, or 5 to 6 membered heteroaryl). In embodiments, R³⁹ is an unsubstituted heteroaryl (e.g., 5 to 10 membered heteroaryl, 5 to 9 membered heteroaryl, or 5 to 6 membered heteroaryl).

In embodiments, R⁵ is substituted or unsubstituted cycloalkyl, substituted or unsubstituted heterocycloalkyl, substituted or unsubstituted aryl, or substituted or unsubstituted heteroaryl.

In embodiments, L¹ is a bond; and R⁵ is a substituted or unsubstituted heterocycloalkyl or a substituted or unsubstituted heteroaryl.

In embodiments, R⁵ is a substituted or unsubstituted 5,6-fused ring system, wherein the 5-membered ring of the 5,6-fused ring system is covalently bound to L¹.

In embodiments, R⁵ is:

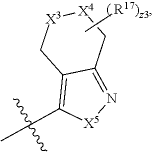

wherein X³, X⁴, X⁵, R¹⁷, and z3 are as described herein, including embodiments.

In embodiments, R⁵ is

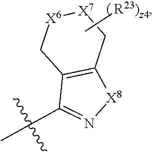

wherein X⁶, X⁷, X⁸, R²³, and z4 are as described herein, including embodiments.

In embodiments, R⁵ is

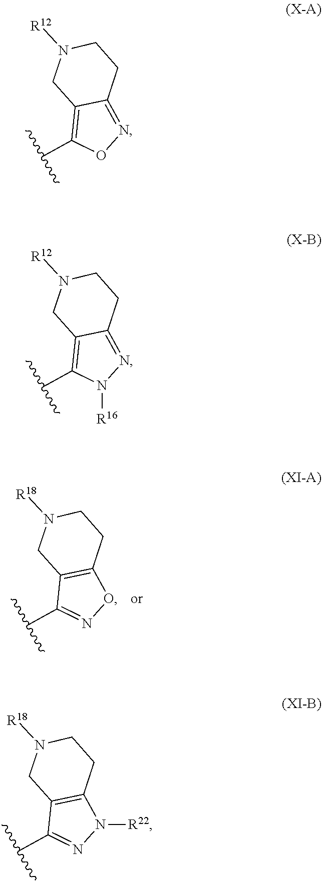

wherein R¹², R¹⁶, R¹⁸, and R²² are as described herein, including embodiments.

In embodiments, R⁵ has the formula

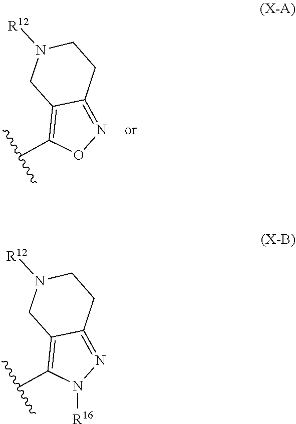

wherein R¹² and R¹⁶ are as described herein, including embodiments.

In embodiments, R⁵ has the formula

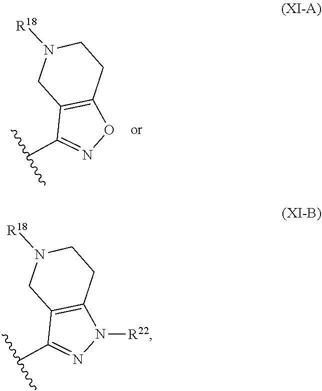

wherein R¹⁸ and R²² are as described herein, including embodiments.

In embodiments, R⁵ is:

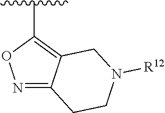

wherein R¹² is as described herein, including embodiments. In embodiments, R⁵ is:

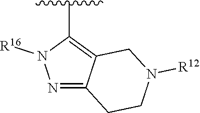

wherein R¹² and R¹⁶ are as described herein, including embodiments. In embodiments, R⁵ is:

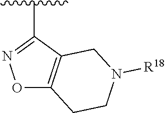

wherein R¹⁸ is as described herein, including embodiments. In embodiments, R⁵ is:

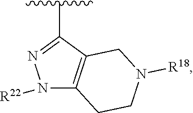

wherein R¹⁸ and R²² are as described herein, including embodiments. In embodiments, R⁵ is

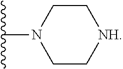 [SMILES: *N1CCNCC1]

In embodiments, R⁵ is

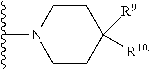

In embodiments, R⁵ is

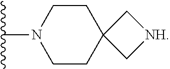 [SMILES: *N1CCC2(CC1)CNC2]

In embodiments, R^{5A}, R^{5B}, R^{5C} and R^{5D} are independently hydrogen, halogen, —CF₃, —CCl₃, —CBr₃, —CI₃, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl); R^{5B} and R^{5C} substituents bonded to the same nitrogen atom may optionally be joined to form a substituted or unsubstituted heterocycloalkyl (e.g., 3 to 8 membered heterocycloalkyl, 3 to 6 membered heterocycloalkyl, or 5 to 6 membered heterocycloalkyl) or substituted or unsubstituted heteroaryl (e.g., 5 to 10 membered heteroaryl, 5 to 9 membered heteroaryl, or 5 to 6 membered heteroaryl).

In embodiments, R^{5A} is hydrogen, halogen, —CF₃, —CCl₃, —CBr₃, —CI₃, —COOH, —CONH₂, R³⁸-substituted or unsubstituted alkyl (e.g., C₁-C₈ alkyl, C₁-C₆ alkyl, or C₁-C₄ alkyl), R³⁸-substituted or unsubstituted heteroalkyl (e.g., 2 to 8 membered heteroalkyl, 2 to 6 membered heteroalkyl, or 2 to 4 membered heteroalkyl), R³⁸-substituted or unsubstituted cycloalkyl (e.g., C₃-C₈ cycloalkyl, C₃-C₆ cycloalkyl, or C₅-C₆ cycloalkyl), R³⁸-substituted or unsubstituted heterocycloalkyl (e.g., 3 to 8 membered heterocycloalkyl, 3 to 6 membered heterocycloalkyl, or 5 to 6 membered heterocycloalkyl), R³⁸-substituted or unsubstituted aryl (e.g., C₆-C₁₀ aryl, C₁₀ aryl, or phenyl), or R³⁸-substituted or unsubstituted heteroaryl (e.g., 5 to 10 membered heteroaryl, 5 to 9 membered heteroaryl, or 5 to 6 membered heteroaryl).

In embodiments, R^{5A} is R³⁸-substituted or unsubstituted alkyl (e.g., C₁-C₈ alkyl, C₁-C₆ alkyl, or C₁-C₄ alkyl). In embodiments, R^{5A} is R³⁸-substituted alkyl (e.g., C₁-C₈ alkyl, C₁-C₆ alkyl, or C₁-C₄ alkyl). In embodiments, R^{5A} is an unsubstituted alkyl (e.g., C₁-C₈ alkyl, C₁-C₆ alkyl, or C₁-C₄ alkyl). In embodiments, R^{5A} is R³⁸-substituted or unsubstituted heteroalkyl (e.g., 2 to 8 membered heteroalkyl, 2 to 6 membered heteroalkyl, or 2 to 4 membered heteroalkyl). In embodiments, R^{5A} is R³⁸-substituted heteroalkyl (e.g., 2 to 8 membered heteroalkyl, 2 to 6 membered heteroalkyl, or 2 to 4 membered heteroalkyl). In embodiments, R^{5A} is an unsubstituted heteroalkyl (e.g., 2 to 8 membered heteroalkyl, 2 to 6 membered heteroalkyl, or 2 to 4 membered heteroalkyl). In embodiments, R^{5A} is R³⁸-substituted or unsubstituted cycloalkyl (e.g., C₃-C₈ cycloalkyl, C₃-C₆ cycloalkyl, or C₅-C₆ cycloalkyl). In embodiments, R^{5A} is R³⁸-substituted cycloalkyl (e.g., C₃-C₈ cycloalkyl, C₃-C₆ cycloalkyl, or C₅-C₆ cycloalkyl). In embodiments, R^{5A} is an unsubstituted cycloalkyl (e.g., C₃-C₈ cycloalkyl, C₃-C₆ cycloalkyl, or C₅-C₆ cycloalkyl). In embodiments, R^{5A} is R³⁸-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{5A} is R³⁸-substituted heterocycloalkyl (e.g., 3 to 8 membered heterocycloalkyl, 3 to 6 membered heterocycloalkyl, or 5 to 6 membered heterocycloalkyl). In embodiments, R^{5A} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{5A} is R³⁸-substituted or unsubstituted aryl (e.g., C₆-C₁₀ aryl, C₁₀ aryl, or phenyl). In embodiments, R^{5A} is R³⁸-substituted aryl (e.g., C₆-C₁₀ aryl, C₁₀ aryl, or phenyl). In embodiments, R^{5A} is an unsubstituted aryl (e.g., C₆-C₁₀ aryl, C₁₀ aryl, or phenyl). In embodiments, R^{5A} is R³⁸-substituted or unsubstituted heteroaryl (e.g., 5 to 10 membered heteroaryl, 5 to 9 membered heteroaryl, or 5 to 6 membered heteroaryl). In embodiments, R^{5A} is R³⁸-substituted heteroaryl (e.g., 5 to 10 membered heteroaryl, 5 to 9 membered heteroaryl, or 5 to 6 membered heteroaryl). In embodiments, R^{5A} is an unsubstituted heteroaryl (e.g., 5 to 10 membered heteroaryl, 5 to 9 membered heteroaryl, or 5 to 6 membered heteroaryl).

In embodiments, R^{5B} is hydrogen, halogen, —CF₃, —CCl₃, —CBr₃, —CI₃, —COOH, —CONH₂, R³⁸-substituted or unsubstituted alkyl (e.g., C₁-C₈ alkyl, C₁-C₆ alkyl, or C₁-C₄ alkyl), R³⁸-substituted or unsubstituted heteroalkyl (e.g., 2 to 8 membered heteroalkyl, 2 to 6 membered heteroalkyl, or 2 to 4 membered heteroalkyl), R³⁸-substituted or unsubstituted cycloalkyl (e.g., C₃-C₈ cycloalkyl, C₃-C₆ cycloalkyl, or C₅-C₆ cycloalkyl), R³⁸-substituted or unsubstituted heterocycloalkyl (e.g., 3 to 8 membered heterocycloalkyl, 3 to 6 membered heterocycloalkyl, or 5 to 6 membered heterocycloalkyl), R³⁸-substituted or unsubstituted aryl (e.g., C₆-C₁₀ aryl, C₁₀ aryl, or phenyl), or R³⁸-substituted or unsubstituted heteroaryl (e.g., 5 to 10 membered heteroaryl, 5 to 9 membered heteroaryl, or 5 to 6 membered heteroaryl). In embodiments, R^{5B} and R^{5C} substituents bonded to the same nitrogen atom may optionally be joined to form a R³⁸-substituted or unsubstituted heterocycloalkyl (e.g., 3 to 8 membered heterocycloalkyl, 3 to 6 membered heterocycloalkyl, or 5 to 6 membered heterocycloalkyl) or R³⁸-substituted or unsubstituted heteroaryl (e.g., 5 to 10 membered heteroaryl, 5 to 9 membered heteroaryl, or 5 to 6 membered heteroaryl).

In embodiments, R^{5B} is R³⁸-substituted or unsubstituted alkyl (e.g., C₁-C₈ alkyl, C₁-C₆ alkyl, or C₁-C₄ alkyl). In embodiments, R^{5B} is R³⁸-substituted alkyl (e.g., C₁-C₈ alkyl, C₁-C₆ alkyl, or C₁-C₄ alkyl). In embodiments, R^{5B} is an unsubstituted alkyl (e.g., C₁-C₈ alkyl, C₁-C₆ alkyl, or C₁-C₄ alkyl). In embodiments, R^{5B} is R³⁸-substituted or unsubstituted heteroalkyl (e.g., 2 to 8 membered heteroalkyl, 2 to 6 membered heteroalkyl, or 2 to 4 membered heteroalkyl). In embodiments, R^{5B} is R³⁸-substituted heteroalkyl (e.g., 2 to 8 membered heteroalkyl, 2 to 6 membered heteroalkyl, or 2 to 4 membered heteroalkyl). In embodiments, R^{5B} is an unsubstituted heteroalkyl (e.g., 2 to 8 membered heteroalkyl, 2 to 6 membered heteroalkyl, or 2 to 4 membered heteroalkyl). In embodiments, R^{5B} is R³⁸-substituted or unsubstituted cycloalkyl (e.g., C₃-C₈ cycloalkyl, C₃-C₆ cycloalkyl, or C₅-C₆ cycloalkyl). In embodiments, R^{5B} is R³⁸-substituted cycloalkyl (e.g., C₃-C₈ cycloalkyl, C₃-C₆ cycloalkyl, or C₅-C₆ cycloalkyl). In embodiments, R^{5B} is an unsubstituted cycloalkyl (e.g., C₃-C₈ cycloalkyl, C₃-C₆ cycloalkyl, or C₅-C₆ cycloalkyl). In embodiments, R^{5B} is R³⁸-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{5B} is R³⁸-substituted heterocycloalkyl (e.g., 3 to 8 membered heterocycloalkyl, 3 to 6 membered heterocycloalkyl, or 5 to 6 membered heterocycloalkyl). In embodiments, R^{5B} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{5B} is R³⁸-substituted or unsubstituted aryl (e.g., C₆-C₁₀ aryl, C₁₀ aryl, or phenyl). In embodiments, R^{5B} is R³⁸-substituted aryl (e.g., C₆-C₁₀ aryl, C₁₀ aryl, or phenyl). In embodiments, R^{5B} is an unsubstituted aryl (e.g., C₆-C₁₀ aryl, C₁₀ aryl, or phenyl). In embodiments, R^{5B} is R³⁸-substituted or unsubstituted heteroaryl (e.g., 5 to 10 membered heteroaryl, 5 to 9 membered heteroaryl, or 5 to 6 membered heteroaryl). In embodiments, R^{5B} is R³⁸-substituted heteroaryl (e.g., 5 to 10 membered heteroaryl, 5 to 9 membered heteroaryl, or 5 to 6 membered heteroaryl). In embodiments, R^{5B} is an unsubstituted heteroaryl (e.g., 5 to 10 membered heteroaryl, 5 to 9 membered heteroaryl, or 5 to 6 membered heteroaryl).

In embodiments, R^{5C} is hydrogen, halogen, —CF₃, —CCl₃, —CBr₃, —CI₃, —COOH, —CONH₂, R³⁸-substituted or unsubstituted alkyl (e.g., C₁-C₈ alkyl, C₁-C₆ alkyl, or C₁-C₄ alkyl), R³⁸-substituted or unsubstituted heteroalkyl (e.g., 2 to 8 membered heteroalkyl, 2 to 6 membered heteroalkyl, or 2 to 4 membered heteroalkyl), R³⁸-substituted or unsubstituted cycloalkyl (e.g., C₃-C₈ cycloalkyl, C₃-C₆ cycloalkyl, or C₅-C₆ cycloalkyl), R³⁸-substituted or unsubstituted heterocycloalkyl (e.g., 3 to 8 membered heterocycloalkyl, 3 to 6 membered heterocycloalkyl, or 5 to 6 membered heterocycloalkyl), R³⁸-substituted or unsubstituted aryl (e.g., C₆-C₁₀ aryl, C₁₀ aryl, or phenyl), or R³⁸-substituted or unsubstituted heteroaryl (e.g., 5 to 10 membered heteroaryl, 5 to 9 membered heteroaryl, or 5 to 6 membered heteroaryl). In embodiments, R^{5B} and R^{5C} substituents bonded to the same nitrogen atom may optionally be joined to form a R³⁸-substituted or unsubstituted heterocycloalkyl (e.g., 3 to 8 membered heterocycloalkyl, 3 to 6 membered heterocycloalkyl, or 5 to 6 membered heterocycloalkyl) or R³⁸-substituted or unsubstituted heteroaryl (e.g., 5 to 10 membered heteroaryl, 5 to 9 membered heteroaryl, or 5 to 6 membered heteroaryl).

In embodiments, R^{5C} is R³⁸-substituted or unsubstituted alkyl (e.g., C₁-C₈ alkyl, C₁-C₆ alkyl, or C₁-C₄ alkyl). In embodiments, R^{5C} is R³⁸-substituted alkyl (e.g., C₁-C₈ alkyl, C₁-C₆ alkyl, or C₁-C₄ alkyl). In embodiments, R^{5C} is an unsubstituted alkyl (e.g., C₁-C₈ alkyl, C₁-C₆ alkyl, or C₁-C₄ alkyl). In embodiments, R^{5C} is R³⁸-substituted or unsubstituted heteroalkyl (e.g., 2 to 8 membered heteroalkyl, 2 to 6 membered heteroalkyl, or 2 to 4 membered heteroalkyl). In embodiments, R^{5C} is R³⁸-substituted heteroalkyl (e.g., 2 to 8 membered heteroalkyl, 2 to 6 membered heteroalkyl, or 2 to 4 membered heteroalkyl). In embodiments, R^{5C} is an unsubstituted heteroalkyl (e.g., 2 to 8 membered heteroalkyl, 2 to 6 membered heteroalkyl, or 2 to 4 membered heteroalkyl). In embodiments, R^{5C} is R³⁸-substituted or unsubstituted cycloalkyl (e.g., C₃-C₈ cycloalkyl, C₃-C₆ cycloalkyl, or C₅-C₆ cycloalkyl). In embodiments, R^{5C} is R³⁸-substituted cycloalkyl (e.g., C₃-C₈ cycloalkyl, C₃-C₆ cycloalkyl, or C₅-C₆ cycloalkyl). In embodiments, R^{5C} is an unsubstituted cycloalkyl (e.g., C₃-C₈ cycloalkyl, C₃-C₆ cycloalkyl, or C₅-C₆ cycloalkyl). In embodiments, R^{5C} is R³⁸-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{5C} is R³⁸-substituted heterocycloalkyl (e.g., 3 to 8 membered heterocycloalkyl, 3 to 6 membered heterocycloalkyl, or 5 to 6 membered heterocycloalkyl). In embodiments, R^{5C} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{5C} is R³⁸-substituted or unsubstituted aryl (e.g., C₆-C₁₀ aryl, C₁₀ aryl, or phenyl). In embodiments, R^{5C} is R³⁸-substituted aryl (e.g., C₆-C₁₀ aryl, C₁₀ aryl, or phenyl). In embodiments, R^{5C} is an unsubstituted aryl (e.g., C₆-C₁₀ aryl, C₁₀ aryl, or phenyl). In embodiments, R^{5C} is R³⁸-substituted or unsubstituted heteroaryl (e.g., 5 to 10 membered heteroaryl, 5 to 9 membered heteroaryl, or 5 to 6 membered heteroaryl). In embodiments, R^{5C} is R³⁸-substituted heteroaryl (e.g., 5 to 10 membered heteroaryl, 5 to 9 membered heteroaryl, or 5 to 6 membered heteroaryl). In embodiments, R^{5C} is an unsubstituted heteroaryl (e.g., 5 to 10 membered heteroaryl, 5 to 9 membered heteroaryl, or 5 to 6 membered heteroaryl).

In embodiments, R^{5D} is hydrogen, halogen, —CF₃, —CCl₃, —CBr₃, —CI₃, —COOH, —CONH₂, R³⁸-substituted or unsubstituted alkyl (e.g., C₁-C₈ alkyl, C₁-C₆ alkyl, or C₁-C₄ alkyl), R³⁸-substituted or unsubstituted heteroalkyl (e.g., 2 to 8 membered heteroalkyl, 2 to 6 membered heteroalkyl, or 2 to 4 membered heteroalkyl), R³⁸-substituted or unsubstituted cycloalkyl (e.g., C₃-C₈ cycloalkyl, C₃-C₆ cycloalkyl, or C₅-C₆ cycloalkyl), R³⁸-substituted or unsubstituted heterocycloalkyl (e.g., 3 to 8 membered heterocycloalkyl, 3 to 6 membered heterocycloalkyl, or 5 to 6 membered heterocycloalkyl), R³⁸-substituted or unsubstituted aryl (e.g., C₆-C₁₀ aryl, C₁₀ aryl, or phenyl), or R³⁸-substituted or unsubstituted heteroaryl (e.g., 5 to 10 membered heteroaryl, 5 to 9 membered heteroaryl, or 5 to 6 membered heteroaryl).

In embodiments, R^{5D} is R³⁸-substituted or unsubstituted alkyl (e.g., C₁-C₈ alkyl, C₁-C₆ alkyl, or C₁-C₄ alkyl). In embodiments, R^{5D} is R³⁸-substituted alkyl (e.g., C₁-C₈ alkyl, C₁-C₆ alkyl, or C₁-C₄ alkyl). In embodiments, R^{5D} is an unsubstituted alkyl (e.g., C₁-C₈ alkyl, C₁-C₆ alkyl, or C₁-C₄ alkyl). In embodiments, R^{5D} is R³⁸-substituted or unsubstituted heteroalkyl (e.g., 2 to 8 membered heteroalkyl, 2 to 6 membered heteroalkyl, or 2 to 4 membered heteroalkyl). In embodiments, R^{5D} is R³⁸-substituted heteroalkyl (e.g., 2 to 8 membered heteroalkyl, 2 to 6 membered heteroalkyl, or 2 to 4 membered heteroalkyl). In embodiments, R^{5D} is an unsubstituted heteroalkyl (e.g., 2 to 8 membered heteroalkyl, 2 to 6 membered heteroalkyl, or 2 to 4 membered heteroalkyl). In embodiments, R^{5D} is R³⁸-substituted or unsubstituted cycloalkyl (e.g., C₃-C₈ cycloalkyl, C₃-C₆ cycloalkyl, or C₅-C₆ cycloalkyl). In embodiments, R^{5D} is R³⁸-substituted cycloalkyl (e.g., C₃-C₈ cycloalkyl, C₃-C₆ cycloalkyl, or C₅-C₆ cycloalkyl). In embodiments, R^{5D} is an unsubstituted cycloalkyl (e.g., C₃-C₈ cycloalkyl, C₃-C₆ cycloalkyl, or C₅-C₆ cycloalkyl). In embodiments, R^{5D} is R³⁸-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{5D} is R³⁸-substituted heterocycloalkyl (e.g., 3 to 8 membered heterocycloalkyl, 3 to 6 membered heterocycloalkyl, or 5 to 6 membered heterocycloalkyl). In embodiments, R^{5D} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{5D} is R³⁸-substituted or unsubstituted aryl (e.g., C₆-C₁₀ aryl, C₁₀ aryl, or phenyl). In embodiments, R^{5D} is R³⁸-substituted aryl (e.g., C₆-C₁₀ aryl, C₁₀ aryl, or phenyl). In embodiments, R^{5D} is an unsubstituted aryl (e.g., C₆-C₁₀ aryl, C₁₀ aryl, or phenyl). In embodiments, R^{5D} is R³⁸-substituted or unsubstituted heteroaryl (e.g., 5 to 10 membered heteroaryl, 5 to 9 membered heteroaryl, or 5 to 6 membered heteroaryl). In embodiments, R^{5D} is R³⁸-substituted heteroaryl (e.g., 5 to 10 membered heteroaryl, 5 to 9 membered heteroaryl, or 5 to 6 membered heteroaryl). In embodiments, R^{5D} is an unsubstituted heteroaryl (e.g., 5 to 10 membered heteroaryl, 5 to 9 membered heteroaryl, or 5 to 6 membered heteroaryl).

In embodiments, R⁵ has the formula:

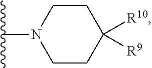

R⁹, wherein R⁹ and R¹⁰ are as described herein, including embodiments. In embodiments, R⁵ has the formula:

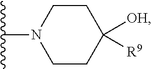

wherein R⁹ is as described herein, including embodiments. In embodiments, R⁵ has the formula:

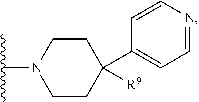

wherein R⁹ is as described herein, including embodiments. In embodiments, R⁵ has the formula:

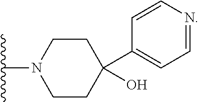 [SMILES: *N1CCC(O)(c2ccncc2)CC1]

In embodiments, R⁵ has the formula:

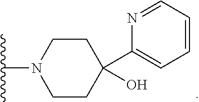 [SMILES: *N1CCC(O)(c2ccccn2)CC1]

In embodiments, R⁵ has the formula:

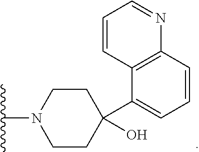 [SMILES: *N1CCC(O)(c2cccc3ncccc23)CC1]

In embodiments, R⁵ has the formula:

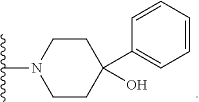 [SMILES: *N1CCC(O)(c2ccccc2)CC1]

In embodiments, R⁵ has the formula:

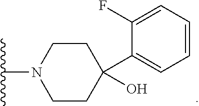 [SMILES: *N1CCC(O)(c2ccccc2F)CC1]

In embodiments, R⁵ has the formula:

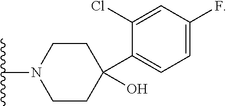 [SMILES: *N1CCC(O)(c2ccc(F)cc2Cl)CC1]

In embodiments, R⁵ has the formula:

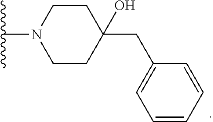 [SMILES: *N1CCC(O)(Cc2ccccc2)CC1]

In embodiments, R⁵ has the formula:

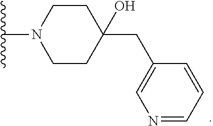 [SMILES: *N1CCC(O)(Cc2cccnc2)CC1]

In embodiments, R⁵ has the formula:

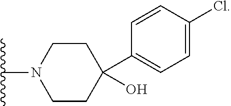 [SMILES: *N1CCC(O)(c2ccc(Cl)cc2)CC1]

In embodiments, R⁵ is:

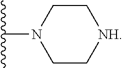 [SMILES: *N1CCNCC1]

In embodiments, R⁵ is:

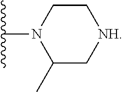 [SMILES: *N1CCNCC1C]

In embodiments, R⁵ is:

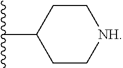 [SMILES: *C1CCNCC1]

In embodiments, R⁵ is:

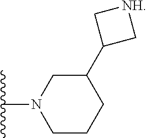 [SMILES: *N1CCCC(C2CNC2)C1]

In embodiments, R⁵ is:

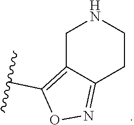 [SMILES: *c1onc2c1CNCC2]

In embodiments, R⁵ is:

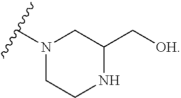 [SMILES: *N1CCNC(CO)C1]

In embodiments, R⁵ is:

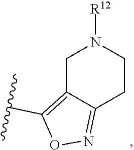

wherein R¹² is as described herein. In embodiments, R⁵ is:

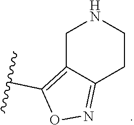 [SMILES: *c1onc2c1CNCC2]

In embodiments, R⁵ is:

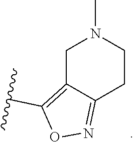 [SMILES: *c1onc2c1CN(C)CC2]

In embodiments, R⁵ is:

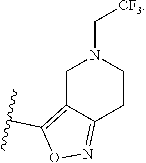 [SMILES: *c1onc2c1CN(CC(F)(F)F)CC2]

In embodiments, R⁵ is:

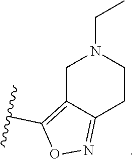 [SMILES: *c1onc2c1CN(CC)CC2]

In embodiments, R⁵ is:

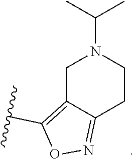 [SMILES: *c1onc2c1CN(C(C)C)CC2]

In embodiments, R⁵ is:

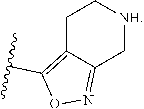 [SMILES: *c1onc2c1CCNC2]

In embodiments, R⁵ is:

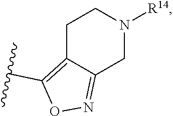

wherein R¹⁴ is as described herein including embodiments. In embodiments, R⁵ is:

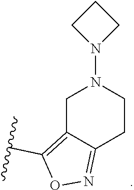 [SMILES: *c1onc2c1CN(N1CCC1)CC2]

In embodiments, R⁵ is:

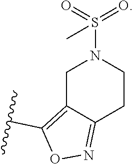 [SMILES: *c1onc2c1CN(S(C)(=O)=O)CC2]

In embodiments, R⁵ is:

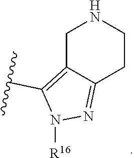

wherein R¹⁶ is as described herein including embodiments. In embodiments, R⁵ is:

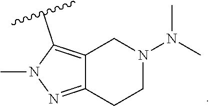

In embodiments, R⁵ is:

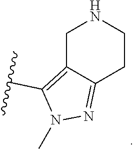 [SMILES: *c1c2c(nn1C)CCNC2]

In embodiments, R⁵ is:

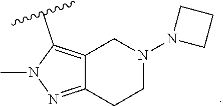 [SMILES: *c1c2c(nn1C)CCN(N1CCC1)C2]

In embodiments, R⁵ is:

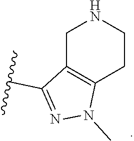 [SMILES: *c1nn(C)c2c1CNCC2]

In embodiments, R⁵ is:

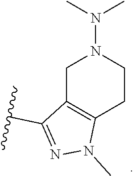 [SMILES: *c1nn(C)c2c1CN(N(C)C)CC2]

In embodiments, R⁵ is:

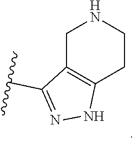 [SMILES: *c1n[nH]c2c1CNCC2]

In embodiments, R⁵ is:

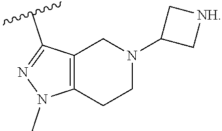 [SMILES: *c1nn(C)c2c1CN(C1CNC1)CC2]

In embodiments, R⁵ is:

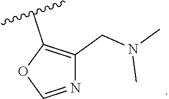 [SMILES: *c1ocnc1CN(C)C]

In embodiments, R⁵ is:

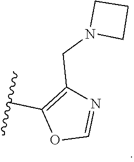 [SMILES: *c1ocnc1CN1CCC1]

In embodiments, R⁵ is:

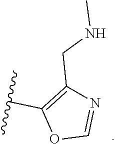 [SMILES: *c1ocnc1CNC]

In embodiments, R⁵ is:

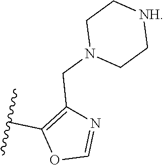 [SMILES: *c1ocnc1CN1CCNCC1]

In embodiments, R⁵ is:

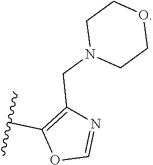 [SMILES: *c1ocnc1CN1CCOCC1]

In embodiments, R⁵ is:

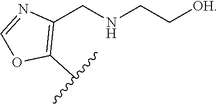 [SMILES: *c1ocnc1CNCCO]

In embodiments, R⁵ is:

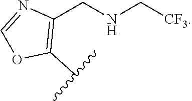 [SMILES: *c1ocnc1CNCC(F)(F)F]

In embodiments, R⁵ is:

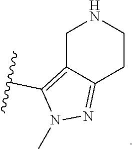 [SMILES: *c1c2c(nn1C)CCNC2]

In embodiments, R⁵ is:

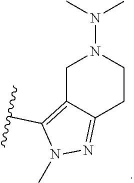 [SMILES: *c1c2c(nn1C)CCN(N(C)C)C2]

In embodiments, R⁵ is:

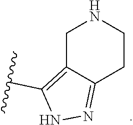 [SMILES: *c1[nH]nc2c1CNCC2]

In embodiments, R⁵ is:

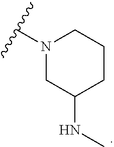 [SMILES: *N1CCCC(NC)C1]

In embodiments, R⁵ is:

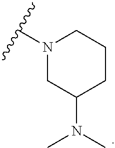 [SMILES: *N1CCCC(N(C)C)C1]

In embodiments, R⁵ is:

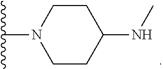 [SMILES: *N1CCC(NC)CC1]

In embodiments, R⁵ is:

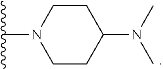 [SMILES: *N1CCC(N(C)C)CC1]

In embodiments, R⁵ is:

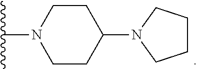 [SMILES: *N1CCC(N2CCCC2)CC1]

In embodiments, R⁵ is:

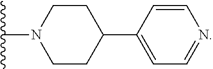 [SMILES: *N1CCC(c2ccncc2)CC1]

In embodiments, R⁵ is:

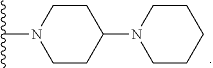 [SMILES: *N1CCC(N2CCCCC2)CC1]

In embodiments, R⁵ is:

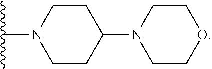 [SMILES: *N1CCC(N2CCOCC2)CC1]

In embodiments, R⁵ is:

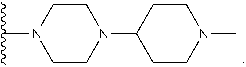 [SMILES: *N1CCN(C2CCN(C)CC2)CC1]

In embodiments, R⁵ is:

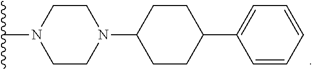 [SMILES: *N1CCN(C2CCC(c3ccccc3)CC2)CC1]

In embodiments, R⁵ is:

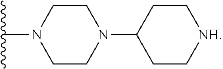 [SMILES: *N1CCN(C2CCNCC2)CC1]

In embodiments, R⁵ is:

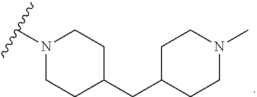 [SMILES: *N1CCC(CC2CCN(C)CC2)CC1]

In embodiments, R⁵ is:

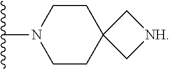 [SMILES: *N1CCC2(CC1)CNC2]

In embodiments, R⁵ is:

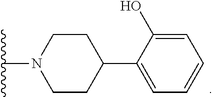 [SMILES: *N1CCC(c2ccccc2O)CC1]

In embodiments, R⁵ is:

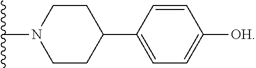 [SMILES: *N1CCC(c2ccc(O)cc2)CC1]

In embodiments, R⁵ is:

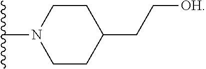 [SMILES: *N1CCC(CCO)CC1]

In embodiments, R⁵ is:

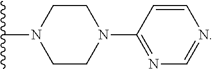 [SMILES: *N1CCN(c2ccncn2)CC1]

In embodiments, R⁵ is:

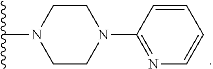 [SMILES: *N1CCN(c2ccccn2)CC1]

In embodiments, R⁵ is:

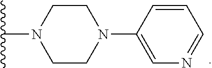 [SMILES: *N1CCN(c2cccnc2)CC1]

In embodiments, R⁵ is:

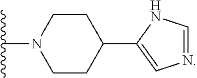 [SMILES: *N1CCC(c2cnc[nH]2)CC1]

In embodiments, R⁵ is:

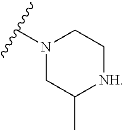 [SMILES: *N1CCNC(C)C1]

In embodiments, R⁵ is:

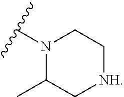 [SMILES: *N1CCNCC1C]

In embodiments, R⁵ is:

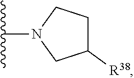

wherein R³⁸ is as described herein including embodiments. In embodiments, R⁵ is:

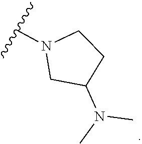 [SMILES: *N1CCC(N(C)C)C1]

In embodiments, R⁵ is:

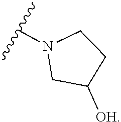 [SMILES: *N1CCC(O)C1]

In embodiments, R⁵ is:

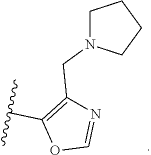 [SMILES: *c1ocnc1CN1CCCC1]

In embodiments, R⁵ is

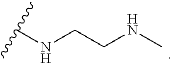 [SMILES: *NCCNC]

In embodiments, R⁵ is:

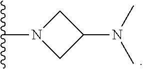 [SMILES: *N1CC(N(C)C)C1]

In embodiments, R⁵ is:

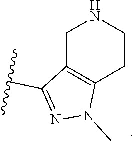 [SMILES: *c1nn(C)c2c1CNCC2]

In embodiments, R⁵ is:

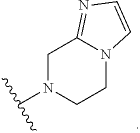 [SMILES: *N1CCn2ccnc2C1]

In embodiments, R⁵ is:

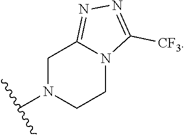 [SMILES: *N1CCn2c(nnc2C(F)(F)F)C1]

In embodiments, R⁵ is:

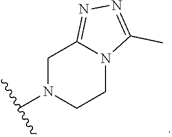 [SMILES: *N1CCn2c(C)nnc2C1]

In embodiments, R⁵ is:

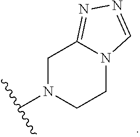 [SMILES: *N1CCn2cnnc2C1]

In embodiments, R⁵ is:

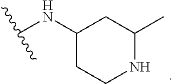 [SMILES: *NC1CCNC(C)C1]

In embodiments, R⁵ is:

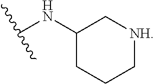 [SMILES: *NC1CCCNC1]

In embodiments, R⁵ is:

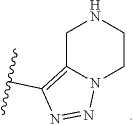 [SMILES: *c1nnn2c1CNCC2]

In embodiments, R⁵ is:

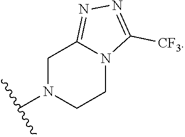 [SMILES: *N1CCn2c(nnc2C(F)(F)F)C1]

In embodiments, R⁵ is:

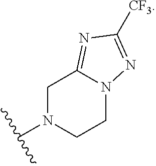 [SMILES: *N1CCn2nc(C(F)(F)F)nc2C1]

In embodiments, R⁶ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₆H, —SOᵥ₆NH₂, —NHNH₂, —ONH₂, —NHC(O)NHNH₂, —NHC(O)NH₂, —N(O)ₘ₆, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, R⁶ is hydrogen.

In embodiments, R⁶ is a substituted or unsubstituted alkyl (e.g., C₁-C₈ alkyl, C₁-C₆ alkyl, or C₁-C₄ alkyl). In embodiments, R⁶ is a substituted alkyl (e.g., C₁-C₈ alkyl, C₁-C₆ alkyl, or C₁-C₄ alkyl). In embodiments, R⁶ is an unsubstituted alkyl (e.g., C₁-C₈ alkyl, C₁-C₆ alkyl, or C₁-C₄ alkyl). In embodiments, R⁶ is a substituted or unsubstituted heteroalkyl (e.g., 2 to 8 membered heteroalkyl, 2 to 6 membered heteroalkyl, or 2 to 4 membered heteroalkyl). In embodiments, R⁶ is a substituted heteroalkyl (e.g., 2 to 8 membered heteroalkyl, 2 to 6 membered heteroalkyl, or 2 to 4 membered heteroalkyl). In embodiments, R⁶ is an unsubstituted heteroalkyl (e.g., 2 to 8 membered heteroalkyl, 2 to 6 membered heteroalkyl, or 2 to 4 membered heteroalkyl). In embodiments, R⁶ is a substituted or unsubstituted cycloalkyl (e.g., C₃-C₈ cycloalkyl, C₃-C₆ cycloalkyl, or C₅-C₆ cycloalkyl). In embodiments, R⁶ is a substituted cycloalkyl (e.g., C₃-C₈ cycloalkyl, C₃-C₆ cycloalkyl, or C₅-C₆ cycloalkyl). In embodiments, R⁶ is an unsubstituted cycloalkyl (e.g., C₃-C₈ cycloalkyl, C₃-C₆ cycloalkyl, or C₅-C₆ cycloalkyl). In embodiments, R⁶ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁶ is a substituted heterocycloalkyl (e.g., 3 to 8 membered heterocycloalkyl, 3 to 6 membered heterocycloalkyl, or 5 to 6 membered heterocycloalkyl). In embodiments, R⁶ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁶ is a substituted or unsubstituted aryl (e.g., C₆-C₁₀ aryl, C₁₀ aryl, or phenyl). In embodiments, R⁶ is a substituted aryl (e.g., C₆-C₁₀ aryl, C₁₀ aryl, or phenyl). In embodiments, R⁶ is an unsubstituted aryl (e.g., C₆-C₁₀ aryl, C₁₀ aryl, or phenyl). In embodiments, R⁶ is a substituted or unsubstituted heteroaryl (e.g., 5 to 10 membered heteroaryl, 5 to 9 membered heteroaryl, or 5 to 6 membered heteroaryl). In embodiments, R⁶ is a substituted heteroaryl (e.g., 5 to 10 membered heteroaryl, 5 to 9 membered heteroaryl, or 5 to 6 membered heteroaryl). In embodiments, R⁶ is an unsubstituted heteroaryl (e.g., 5 to 10 membered heteroaryl, 5 to 9 membered heteroaryl, or 5 to 6 membered heteroaryl).

In embodiments, R⁷ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₇H, —SOᵥ₇NH₂, —NHNH₂, —ONH₂, —NHC(O)NHNH₂, —NHC(O)NH₂, —N(O)ₘ₇, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R⁷ is a substituted or unsubstituted alkyl (e.g., C₁-C₈ alkyl, C₁-C₆ alkyl, or C₁-C₄ alkyl). In embodiments, R⁷ is a substituted alkyl (e.g., C₁-C₈ alkyl, C₁-C₆ alkyl, or C₁-C₄ alkyl). In embodiments, R⁷ is an unsubstituted alkyl (e.g., C₁-C₈ alkyl, C₁-C₆ alkyl, or C₁-C₄ alkyl). In embodiments, R⁷ is a substituted or unsubstituted heteroalkyl (e.g., 2 to 8 membered heteroalkyl, 2 to 6 membered heteroalkyl, or 2 to 4 membered heteroalkyl). In embodiments, R⁷ is a substituted heteroalkyl (e.g., 2 to 8 membered heteroalkyl, 2 to 6 membered heteroalkyl, or 2 to 4 membered heteroalkyl). In embodiments, R⁷ is an unsubstituted heteroalkyl (e.g., 2 to 8 membered heteroalkyl, 2 to 6 membered heteroalkyl, or 2 to 4 membered heteroalkyl). In embodiments, R⁷ is a substituted or unsubstituted cycloalkyl (e.g., C₃-C₈ cycloalkyl, C₃-C₆ cycloalkyl, or C₅-C₆ cycloalkyl). In embodiments, R⁷ is a substituted cycloalkyl (e.g., C₃-C₈ cycloalkyl, C₃-C₆ cycloalkyl, or C₅-C₆ cycloalkyl). In embodiments, R⁷ is an unsubstituted cycloalkyl (e.g., C₃-C₈ cycloalkyl, C₃-C₆ cycloalkyl, or C₅-C₆ cycloalkyl). In embodiments, R⁷ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁷ is a substituted heterocycloalkyl (e.g., 3 to 8 membered heterocycloalkyl, 3 to 6 membered heterocycloalkyl, or 5 to 6 membered heterocycloalkyl). In embodiments, R⁷ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁷ is a substituted or unsubstituted aryl (e.g., C₆-C₁₀ aryl, C₁₀ aryl, or phenyl). In embodiments, R⁷ is a substituted aryl (e.g., C₆-C₁₀ aryl, C₁₀ aryl, or phenyl). In embodiments, R⁷ is an unsubstituted aryl (e.g., C₆-C₁₀ aryl, C₁₀ aryl, or phenyl). In embodiments, R⁷ is a substituted or unsubstituted heteroaryl (e.g., 5 to 10 membered heteroaryl, 5 to 9 membered heteroaryl, or 5 to 6 membered heteroaryl). In embodiments, R⁷ is a substituted heteroaryl (e.g., 5 to 10 membered heteroaryl, 5 to 9 membered heteroaryl, or 5 to 6 membered heteroaryl). In embodiments, R⁷ is an unsubstituted heteroaryl (e.g., 5 to 10 membered heteroaryl, 5 to 9 membered heteroaryl, or 5 to 6 membered heteroaryl).

In embodiments, R⁸ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₅H, —SOᵥ₅NH₂, —NHNH₂, —ONH₂, —NHC(O)NHNH₂, —NHC(O)NH₂, —N(O)ₘ₅, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, R⁸ is hydrogen.

In embodiments, R⁸ is a substituted or unsubstituted alkyl (e.g., C₁-C₈ alkyl, C₁-C₆ alkyl, or C₁-C₄ alkyl). In embodiments, R⁸ is a substituted alkyl (e.g., C₁-C₈ alkyl, C₁-C₆ alkyl, or C₁-C₄ alkyl). In embodiments, R⁸ is an unsubstituted alkyl (e.g., C₁-C₈ alkyl, C₁-C₆ alkyl, or C₁-C₄ alkyl). In embodiments, R⁸ is a substituted or unsubstituted heteroalkyl (e.g., 2 to 8 membered heteroalkyl, 2 to 6 membered heteroalkyl, or 2 to 4 membered heteroalkyl). In embodiments, R⁸ is a substituted heteroalkyl (e.g., 2 to 8 membered heteroalkyl, 2 to 6 membered heteroalkyl, or 2 to 4 membered heteroalkyl). In embodiments, R⁸ is an unsubstituted heteroalkyl (e.g., 2 to 8 membered heteroalkyl, 2 to 6 membered heteroalkyl, or 2 to 4 membered heteroalkyl). In embodiments, R⁸ is a substituted or unsubstituted cycloalkyl (e.g., C₃-C₈ cycloalkyl, C₃-C₆ cycloalkyl, or C₅-C₆ cycloalkyl). In embodiments, R⁸ is a substituted cycloalkyl (e.g., C₃-C₈ cycloalkyl, C₃-C₆ cycloalkyl, or C₅-C₆ cycloalkyl). In embodiments, R⁸ is an unsubstituted cycloalkyl (e.g., C₃-C₈ cycloalkyl, C₃-C₆ cycloalkyl, or C₅-C₆ cycloalkyl). In embodiments, R⁸ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁸ is a substituted heterocycloalkyl (e.g., 3 to 8 membered heterocycloalkyl, 3 to 6 membered heterocycloalkyl, or 5 to 6 membered heterocycloalkyl). In embodiments, R⁸ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁸ is a substituted or unsubstituted aryl (e.g., C₆-C₁₀ aryl, C₁₀ aryl, or phenyl). In embodiments, R⁸ is a substituted aryl (e.g., C₆-C₁₀ aryl, C₁₀ aryl, or phenyl). In embodiments, R⁸ is an unsubstituted aryl (e.g., C₆-C₁₀ aryl, C₁₀ aryl, or phenyl). In embodiments, R⁸ is a substituted or unsubstituted heteroaryl (e.g., 5 to 10 membered heteroaryl, 5 to 9 membered heteroaryl, or 5 to 6 membered heteroaryl). In embodiments, R⁸ is a substituted heteroaryl (e.g., 5 to 10 membered heteroaryl, 5 to 9 membered heteroaryl, or 5 to 6 membered heteroaryl). In embodiments, R⁸ is an unsubstituted heteroaryl (e.g., 5 to 10 membered heteroaryl, 5 to 9 membered heteroaryl, or 5 to 6 membered heteroaryl).

In embodiments, R⁹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₉R^{9A}, —SOᵥ₉NR^{9B}R^{9C}, —NHNR^{9B}R^{9C}, —ONR^{9B}R^{9C}, —NHC(O)NHNR^{9B}R^{9C}, —NHC(O)NR^{9B}R^{9C}, —N(O)ₘ₉, —NR^{9B}R^{9C}, —C(O)R^{9D}, —C(O)OR^{9D}, —C(O)NR^{9B}R^{9C}, —OR^{9A}, —NR^{9B}SO₂R^{9A}, —NR^{9B}C(O)R^{9D}, —NR^{9B}C(O)OR^{9D}, —NR^{9B}OR^{9D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, R⁹ is hydrogen.

In embodiments, R⁹ is substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, R⁹ is substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, R⁹ is substituted or unsubstituted pyridinyl.

In embodiments, R⁹ and R¹⁰ substituents may optionally be joined to form a substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R⁹ and R¹⁰ substituents may optionally be joined to form a R⁴⁻substituted or unsubstituted cycloalkyl (e.g., C₃-C₈ cycloalkyl, C₃-C₆ cycloalkyl, or C₅-C₆ cycloalkyl), R⁴¹-substituted or unsubstituted heterocycloalkyl (e.g., 3 to 8 membered heterocycloalkyl, 3 to 6 membered heterocycloalkyl, or 5 to 6 membered heterocycloalkyl), R⁴¹-substituted or unsubstituted aryl (e.g., C₆-C₁₀ aryl, C₁₀ aryl, or phenyl), or R⁴¹-substituted or unsubstituted heteroaryl (e.g., 5 to 10 membered heteroaryl, 5 to 9 membered heteroaryl, or 5 to 6 membered heteroaryl).

In embodiments, R⁹ and R¹⁰ substituents may optionally be joined to form a R⁴¹⁻substituted cycloalkyl (e.g., C₃-C₈ cycloalkyl, C₃-C₆ cycloalkyl, or C₅-C₆ cycloalkyl), R⁴¹-substituted heterocycloalkyl (e.g., 3 to 8 membered heterocycloalkyl, 3 to 6 membered heterocycloalkyl, or 5 to 6 membered heterocycloalkyl), R⁴¹-substituted aryl (e.g., C₆-C₁₀ aryl, C₁₀ aryl, or phenyl), or R⁴¹-substituted heteroaryl (e.g., 5 to 10 membered heteroaryl, 5 to 9 membered heteroaryl, or 5 to 6 membered heteroaryl). In embodiments, R⁹ and R¹⁰ substituents may optionally be joined to form an unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R⁹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₉R^{9A}, —SOᵥ₉NR^{9B}R^{9C}, —NHNR^{9B}R^{9C}, —ONR^{9B}R^{9C}, —NHC(O)NHNR^{9B}R^{9C}, —NHC(O)NR^{9B}R^{9C}, —N(O)ₘ₉, —NR^{9B}R^{9C}, —C(O)R^{9D}, —C(O)OR^{9D}, —C(O)NR^{9B}R^{9C}, —OR^{9A}, —NR^{9B}SO₂R^{9A}, —NR^{9B}C(O)R^{9D}, —NR^{9B}C(O)OR^{9D}, —NR^{9B}OR^{9D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R⁴¹-substituted or unsubstituted alkyl (e.g., C₁-C₈ alkyl, C₁-C₆ alkyl, or C₁-C₄ alkyl), R⁴¹-substituted or unsubstituted heteroalkyl (e.g., 2 to 8 membered heteroalkyl, 2 to 6 membered heteroalkyl, or 2 to 4 membered heteroalkyl), R⁴¹-substituted or unsubstituted cycloalkyl (e.g., C₃-C₈ cycloalkyl, C₃-C₆ cycloalkyl, or C₅-C₆ cycloalkyl), R⁴¹-substituted or unsubstituted heterocycloalkyl (e.g., 3 to 8 membered heterocycloalkyl, 3 to 6 membered heterocycloalkyl, or 5 to 6 membered heterocycloalkyl), R⁴¹-substituted or unsubstituted aryl (e.g., C₆-C₁₀ aryl, C₁₀ aryl, or phenyl), or R⁴¹-substituted or unsubstituted heteroaryl (e.g., 5 to 10 membered heteroaryl, 5 to 9 membered heteroaryl, or 5 to 6 membered heteroaryl).

In embodiments, R⁹ is R⁴¹-substituted or unsubstituted alkyl (e.g., C₁-C₈ alkyl, C₁-C₆ alkyl, or C₁-C₄ alkyl). In embodiments, R⁹ is R⁴¹-substituted alkyl (e.g., C₁-C₈ alkyl, C₁-C₆ alkyl, or C₁-C₄ alkyl). In embodiments, R⁹ is an unsubstituted alkyl (e.g., C₁-C₈ alkyl, C₁-C₆ alkyl, or C₁-C₄ alkyl). In embodiments, R⁹ is R⁴¹-substituted or unsubstituted heteroalkyl (e.g., 2 to 8 membered heteroalkyl, 2 to 6 membered heteroalkyl, or 2 to 4 membered heteroalkyl). In embodiments, R⁹ is R⁴¹-substituted heteroalkyl (e.g., 2 to 8 membered heteroalkyl, 2 to 6 membered heteroalkyl, or 2 to 4 membered heteroalkyl). In embodiments, R⁹ is an unsubstituted heteroalkyl (e.g., 2 to 8 membered heteroalkyl, 2 to 6 membered heteroalkyl, or 2 to 4 membered heteroalkyl). In embodiments, R⁹ is R⁴¹-substituted or unsubstituted cycloalkyl (e.g., C₃-C₈ cycloalkyl, C₃-C₆ cycloalkyl, or C₅-C₆ cycloalkyl). In embodiments, R⁹ is R⁴¹-substituted cycloalkyl (e.g., C₃-C₈ cycloalkyl, C₃-C₆ cycloalkyl, or C₅-C₆ cycloalkyl). In embodiments, R⁹ is an unsubstituted cycloalkyl (e.g., C₃-C₈ cycloalkyl, C₃-C₆ cycloalkyl, or C₅-C₆ cycloalkyl). In embodiments, R⁹ is R⁴¹-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁹ is R⁴¹-substituted heterocycloalkyl (e.g., 3 to 8 membered heterocycloalkyl, 3 to 6 membered heterocycloalkyl, or 5 to 6 membered heterocycloalkyl). In embodiments, R⁹ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁹ is R⁴¹-substituted or unsubstituted aryl (e.g., C₆-C₁₀ aryl, C₁₀ aryl, or phenyl). In embodiments, R⁹ is R⁴¹-substituted aryl (e.g., C₆-C₁₀ aryl, C₁₀ aryl, or phenyl). In embodiments, R⁹ is an unsubstituted aryl (e.g., C₆-C₁₀ aryl, C₁₀ aryl, or phenyl). In embodiments, R⁹ is R⁴¹-substituted or unsubstituted heteroaryl (e.g., 5 to 10 membered heteroaryl, 5 to 9 membered heteroaryl, or 5 to 6 membered heteroaryl). In embodiments, R⁹ is R⁴¹-substituted heteroaryl (e.g., 5 to 10 membered heteroaryl, 5 to 9 membered heteroaryl, or 5 to 6 membered heteroaryl). In embodiments, R⁹ is an unsubstituted heteroaryl (e.g., 5 to 10 membered heteroaryl, 5 to 9 membered heteroaryl, or 5 to 6 membered heteroaryl).

R⁴¹ is independently oxo, halogen, —CCl₃, —CBr₃, —CF₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂F, —OCH₂Cl, —OCH₂Br, —OCH₂I, R⁴²-substituted or unsubstituted alkyl (e.g., C₁-C₈ alkyl, C₁-C₆ alkyl, or C₁-C₄ alkyl), R⁴²-substituted or unsubstituted heteroalkyl (e.g., 2 to 8 membered heteroalkyl, 2 to 6 membered heteroalkyl, or 2 to 4 membered heteroalkyl), R⁴²-substituted or unsubstituted cycloalkyl (e.g., C₃-C₈ cycloalkyl, C₃-C₆ cycloalkyl, or C₅-C₆ cycloalkyl), R⁴²-substituted or unsubstituted heterocycloalkyl (e.g., 3 to 8 membered heterocycloalkyl, 3 to 6 membered heterocycloalkyl, or 5 to 6 membered heterocycloalkyl), R⁴²-substituted or unsubstituted aryl (e.g., C₆-C₁₀ aryl, C₁₀ aryl, or phenyl), or R⁴²-substituted or unsubstituted heteroaryl (e.g., 5 to 10 membered heteroaryl, 5 to 9 membered heteroaryl, or 5 to 6 membered heteroaryl).

In embodiments, R⁴¹ is R⁴²-substituted or unsubstituted alkyl (e.g., C₁-C₈ alkyl, C₁-C₆ alkyl, or C₁-C₄ alkyl). In embodiments, R⁴¹ is R⁴²-substituted alkyl (e.g., C₁-C₈ alkyl, C₁-C₆ alkyl, or C₁-C₄ alkyl). In embodiments, R⁴¹ is an unsubstituted alkyl (e.g., C₁-C₈ alkyl, C₁-C₆ alkyl, or C₁-C₄ alkyl). In embodiments, R⁴¹ is R⁴²-substituted or unsubstituted heteroalkyl (e.g., 2 to 8 membered heteroalkyl, 2 to 6 membered heteroalkyl, or 2 to 4 membered heteroalkyl). In embodiments, R⁴¹ is R⁴²-substituted heteroalkyl (e.g., 2 to 8 membered heteroalkyl, 2 to 6 membered heteroalkyl, or 2 to 4 membered heteroalkyl). In embodiments, R⁴¹ is an unsubstituted heteroalkyl (e.g., 2 to 8 membered heteroalkyl, 2 to 6 membered heteroalkyl, or 2 to 4 membered heteroalkyl). In embodiments, R⁴¹ is R⁴²-substituted or unsubstituted cycloalkyl (e.g., C₃-C₈ cycloalkyl, C₃-C₆ cycloalkyl, or C₅-C₆ cycloalkyl). In embodiments, R⁴¹ is R⁴²-substituted cycloalkyl (e.g., C₃-C₈ cycloalkyl, C₃-C₆ cycloalkyl, or C₅-C₆ cycloalkyl). In embodiments, R⁴¹ is an unsubstituted cycloalkyl (e.g., C₃-C₈ cycloalkyl, C₃-C₆ cycloalkyl, or C₅-C₆ cycloalkyl). In embodiments, R⁴¹ is R⁴²-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴¹ is R⁴²-substituted heterocycloalkyl (e.g., 3 to 8 membered heterocycloalkyl, 3 to 6 membered heterocycloalkyl, or 5 to 6 membered heterocycloalkyl). In embodiments, R⁴¹ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁴¹ is R⁴²-substituted or unsubstituted aryl (e.g., C₆-C₁₀ aryl, C₁₀ aryl, or phenyl). In embodiments, R⁴¹ is R⁴²-substituted aryl (e.g., C₆-C₁₀ aryl, C₁₀ aryl, or phenyl). In embodiments, R⁴¹ is an unsubstituted aryl (e.g., C₆-C₁₀ aryl, C₁₀ aryl, or phenyl). In embodiments, R⁴¹ is R⁴²-substituted or unsubstituted heteroaryl (e.g., 5 to 10 membered heteroaryl, 5 to 9 membered heteroaryl, or 5 to 6 membered heteroaryl). In embodiments, R⁴¹ is R⁴²-substituted heteroaryl (e.g., 5 to 10 membered heteroaryl, 5 to 9 membered heteroaryl, or 5 to 6 membered heteroaryl). In embodiments, R⁴¹ is an unsubstituted heteroaryl (e.g., 5 to 10 membered heteroaryl, 5 to 9 membered heteroaryl, or 5 to 6 membered heteroaryl).

R⁴² is independently oxo, halogen, —CCl₃, —CBr₃, —CF₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂F, —OCH₂Cl, —OCH₂Br, —OCH₂I, R⁴³-substituted or unsubstituted alkyl (e.g., C₁-C₈ alkyl, C₁-C₆ alkyl, or C₁-C₄ alkyl), R⁴³-substituted or unsubstituted heteroalkyl (e.g., 2 to 8 membered heteroalkyl, 2 to 6 membered heteroalkyl, or 2 to 4 membered heteroalkyl), R⁴³-substituted or unsubstituted cycloalkyl (e.g., C₃-C₈ cycloalkyl, C₃-C₆ cycloalkyl, or C₅-C₆ cycloalkyl), R⁴³-substituted or unsubstituted heterocycloalkyl (e.g., 3 to 8 membered heterocycloalkyl, 3 to 6 membered heterocycloalkyl, or 5 to 6 membered heterocycloalkyl), R⁴³-substituted or unsubstituted aryl (e.g., C₆-C₁₀ aryl, C₁₀ aryl, or phenyl), or R⁴³-substituted or unsubstituted heteroaryl (e.g., 5 to 10 membered heteroaryl, 5 to 9 membered heteroaryl, or 5 to 6 membered heteroaryl).

In embodiments, R⁴² is R⁴³-substituted or unsubstituted alkyl (e.g., C₁-C₈ alkyl, C₁-C₆ alkyl, or C₁-C₄ alkyl). In embodiments, R⁴² is R⁴³-substituted alkyl (e.g., C₁-C₈ alkyl, C₁-C₆ alkyl, or C₁-C₄ alkyl). In embodiments, R⁴² is an unsubstituted alkyl (e.g., C₁-C₈ alkyl, C₁-C₆ alkyl, or C₁-C₄ alkyl). In embodiments, R⁴² is R⁴³-substituted or unsubstituted heteroalkyl (e.g., 2 to 8 membered heteroalkyl, 2 to 6 membered heteroalkyl, or 2 to 4 membered heteroalkyl). In embodiments, R⁴² is R⁴³-substituted heteroalkyl (e.g., 2 to 8 membered heteroalkyl, 2 to 6 membered heteroalkyl, or 2 to 4 membered heteroalkyl). In embodiments, R⁴² is an unsubstituted heteroalkyl (e.g., 2 to 8 membered heteroalkyl, 2 to 6 membered heteroalkyl, or 2 to 4 membered heteroalkyl). In embodiments, R⁴² is R⁴³-substituted or unsubstituted cycloalkyl (e.g., C₃-C₈ cycloalkyl, C₃-C₆ cycloalkyl, or C₅-C₆ cycloalkyl). In embodiments, R⁴² is R⁴³-substituted cycloalkyl (e.g., C₃-C₈ cycloalkyl, C₃-C₆ cycloalkyl, or C₅-C₆ cycloalkyl). In embodiments, R⁴² is an unsubstituted cycloalkyl (e.g., C₃-C₈ cycloalkyl, C₃-C₆ cycloalkyl, or C₅-C₆ cycloalkyl). In embodiments, R⁴² is R⁴³-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴² is R⁴³-substituted heterocycloalkyl (e.g., 3 to 8 membered heterocycloalkyl, 3 to 6 membered heterocycloalkyl, or 5 to 6 membered heterocycloalkyl). In embodiments, R⁴² is an unsubstituted heterocycloalkyl (e.g., 3 to 8 membered heterocycloalkyl, 3 to 6 membered heterocycloalkyl, or 5 to 6 membered heterocycloalkyl). In embodiments, R⁴² is R⁴³-substituted or unsubstituted aryl (e.g., C₆-C₁₀ aryl, C₁₀ aryl, or phenyl). In embodiments, R⁴² is R⁴³-substituted aryl (e.g., C₆-C₁₀ aryl, C₁₀ aryl, or phenyl). In embodiments, R⁴² is an unsubstituted aryl (e.g., C₆-C₁₀ aryl, C₁₀ aryl, or phenyl). In embodiments, R⁴² is R⁴³-substituted or unsubstituted heteroaryl (e.g., 5 to 10 membered heteroaryl, 5 to 9 membered heteroaryl, or 5 to 6 membered heteroaryl). In embodiments, R⁴² is R⁴³-substituted heteroaryl (e.g., 5 to 10 membered heteroaryl, 5 to 9 membered heteroaryl, or 5 to 6 membered heteroaryl). In embodiments, R⁴² is an unsubstituted heteroaryl (e.g., 5 to 10 membered heteroaryl, 5 to 9 membered heteroaryl, or 5 to 6 membered heteroaryl).

In embodiments, R^{9A}, R^{9B}, R^{9C}, and R^{9D} are independently hydrogen, halogen, —CF₃, —CCl₃, —CBr₃, —CI₃, —COOH, —CONH₂, s substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl); R^{9B} and R^{9C} substituents bonded to the same nitrogen atom may optionally be joined to form a substituted or unsubstituted heterocycloalkyl (e.g., 3 to 8 membered heterocycloalkyl, 3 to 6 membered heterocycloalkyl, or 5 to 6 membered heterocycloalkyl) or substituted or unsubstituted heteroaryl (e.g., 5 to 10 membered heteroaryl, 5 to 9 membered heteroaryl, or 5 to 6 membered heteroaryl).

In embodiments, R^{9A} is hydrogen, halogen, —CF₃, —CCl₃, —CBr₃, —CI₃, —COOH, —CONH₂, R⁴¹-substituted or unsubstituted alkyl (e.g., C₁-C₈ alkyl, C₁-C₆ alkyl, or C₁-C₄ alkyl), R⁴¹-substituted or unsubstituted heteroalkyl (e.g., 2 to 8 membered heteroalkyl, 2 to 6 membered heteroalkyl, or 2 to 4 membered heteroalkyl), R⁴¹-substituted or unsubstituted cycloalkyl (e.g., C₃-C₈ cycloalkyl, C₃-C₆ cycloalkyl, or C₅-C₆ cycloalkyl), R⁴¹-substituted or unsubstituted heterocycloalkyl (e.g., 3 to 8 membered heterocycloalkyl, 3 to 6 membered heterocycloalkyl, or 5 to 6 membered heterocycloalkyl), R⁴¹-substituted or unsubstituted aryl (e.g., C₆-C₁₀ aryl, C₁₀ aryl, or phenyl), or R⁴¹-substituted or unsubstituted heteroaryl (e.g., 5 to 10 membered heteroaryl, 5 to 9 membered heteroaryl, or 5 to 6 membered heteroaryl).

In embodiments, R^{9B} is hydrogen, halogen, —CF₃, —CCl₃, —CBr₃, —CI₃, —COOH, —CONH₂, R⁴¹-substituted or unsubstituted alkyl (e.g., C₁-C₈ alkyl, C₁-C₆ alkyl, or C₁-C₄ alkyl), R⁴¹-substituted or unsubstituted heteroalkyl (e.g., 2 to 8 membered heteroalkyl, 2 to 6 membered heteroalkyl, or 2 to 4 membered heteroalkyl), R⁴¹-substituted or unsubstituted cycloalkyl (e.g., C₃-C₈ cycloalkyl, C₃-C₆ cycloalkyl, or C₅-C₆ cycloalkyl), R⁴¹-substituted or unsubstituted heterocycloalkyl (e.g., 3 to 8 membered heterocycloalkyl, 3 to 6 membered heterocycloalkyl, or 5 to 6 membered heterocycloalkyl), R⁴¹-substituted or unsubstituted aryl (e.g., C₆-C₁₀ aryl, C₁₀ aryl, or phenyl), or R⁴¹-substituted or unsubstituted heteroaryl (e.g., 5 to 10 membered heteroaryl, 5 to 9 membered heteroaryl, or 5 to 6 membered heteroaryl). In embodiments, R^{9B} and R^{9C} substituents bonded to the same nitrogen atom may optionally be joined to form a R⁴¹-substituted or unsubstituted heterocycloalkyl (e.g., 3 to 8 membered heterocycloalkyl, 3 to 6 membered heterocycloalkyl, or 5 to 6 membered heterocycloalkyl) or R⁴¹-substituted or unsubstituted heteroaryl (e.g., 5 to 10 membered heteroaryl, 5 to 9 membered heteroaryl, or 5 to 6 membered heteroaryl).

In embodiments, R^{9C} is hydrogen, halogen, —CF₃, —CCl₃, —CBr₃, —CI₃, —COOH, —CONH₂, R⁴¹-substituted or unsubstituted alkyl (e.g., C₁-C₈ alkyl, C₁-C₆ alkyl, or C₁-C₄ alkyl), R⁴¹-substituted or unsubstituted heteroalkyl (e.g., 2 to 8 membered heteroalkyl, 2 to 6 membered heteroalkyl, or 2 to 4 membered heteroalkyl), R⁴¹-substituted or unsubstituted cycloalkyl (e.g., C₃-C₈ cycloalkyl, C₃-C₆ cycloalkyl, or C₅-C₆ cycloalkyl), R⁴¹-substituted or unsubstituted heterocycloalkyl (e.g., 3 to 8 membered heterocycloalkyl, 3 to 6 membered heterocycloalkyl, or 5 to 6 membered heterocycloalkyl), R⁴¹-substituted or unsubstituted aryl (e.g., C₆-C₁₀ aryl, C₁₀ aryl, or phenyl), or R⁴¹-substituted or unsubstituted heteroaryl (e.g., 5 to 10 membered heteroaryl, 5 to 9 membered heteroaryl, or 5 to 6 membered heteroaryl). In embodiments, R^{9B} and R^{9C} substituents bonded to the same nitrogen atom may optionally be joined to form a R⁴¹⁻substituted or unsubstituted heterocycloalkyl (e.g., 3 to 8 membered heterocycloalkyl, 3 to 6 membered heterocycloalkyl, or 5 to 6 membered heterocycloalkyl) or R⁴¹-substituted or unsubstituted heteroaryl (e.g., 5 to 10 membered heteroaryl, 5 to 9 membered heteroaryl, or 5 to 6 membered heteroaryl).

In embodiments, R^{9D} is hydrogen, halogen, —CF₃, —CCl₃, —CBr₃, —CI₃, —COOH, —CONH₂, R⁴¹-substituted or unsubstituted alkyl (e.g., C₁-C₈ alkyl, C₁-C₆ alkyl, or C₁-C₄ alkyl), R⁴¹-substituted or unsubstituted heteroalkyl (e.g., 2 to 8 membered heteroalkyl, 2 to 6 membered heteroalkyl, or 2 to 4 membered heteroalkyl), R⁴¹-substituted or unsubstituted cycloalkyl (e.g., C₃-C₈ cycloalkyl, C₃-C₆ cycloalkyl, or C₅-C₆ cycloalkyl), R⁴¹-substituted or unsubstituted heterocycloalkyl (e.g., 3 to 8 membered heterocycloalkyl, 3 to 6 membered heterocycloalkyl, or 5 to 6 membered heterocycloalkyl), R⁴¹-substituted or unsubstituted aryl (e.g., C₆-C₁₀ aryl, C₁₀ aryl, or phenyl), or R⁴¹-substituted or unsubstituted heteroaryl (e.g., 5 to 10 membered heteroaryl, 5 to 9 membered heteroaryl, or 5 to 6 membered heteroaryl).

In embodiments, R⁹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁹ is substituted or unsubstituted cycloalkyl, substituted or unsubstituted heterocycloalkyl, substituted or unsubstituted aryl, or substituted or unsubstituted heteroaryl.

In embodiments, R⁹ is substituted or unsubstituted pyridinyl.

In embodiments, R¹⁰ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₀R^{10A}, —SOᵥ₁₀NR^{11B}R^{10C}, —NHNR^{10B}R^{10C}, —ONR^{10B}R^{10C}, —NHC(O)NHNR^{10B}R^{10C}, —NHC(O)NR^{10B}R^{10C}, —N(O)ₘ₁₀, —NR^{10B}R^{10C}, —C(O)R^{10D}, —C(O)OR^{11D}, —C(O)NR^{1B}R^{1C}, —OR^{10A}, —NR^{10B}SO₂R^{10A}, —NR^{10B}C(O)R^{10D}, —NR^{10B}C(O)OR^{10D}, —NR^{10B}OR^{10D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, R¹⁰ is hydrogen.

In embodiments, R¹⁰ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₀R^{10A}, —SOᵥ₁₀NR^{10B}R^{10C}, —NHNR^{10B}R^{10C}, —ONR^{10B}R^{1C}, —NHC(O)NHNR^{10B}R^{10C}, —NHC(O)NR^{10B}R^{10C}, —N(O)ₘ₁₀, —NR^{10B}R^{10C}, —C(O)R^{10D}, —C(O)OR^{10D}, —C(O)NR^{10B}R^{10C}, —OR^{10A}, —NR^{10B}SO₂R^{10A}, —NR^{10B}C(O)R^{10D}, —NR^{10B}C(O)OR^{10D}, —NR^{10B}OR^{10D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R⁴⁴-substituted or unsubstituted alkyl (e.g., C₁-C₈ alkyl, C₁-C₆ alkyl, or C₁-C₄ alkyl), R⁴⁴-substituted or unsubstituted heteroalkyl (e.g., 2 to 8 membered heteroalkyl, 2 to 6 membered heteroalkyl, or 2 to 4 membered heteroalkyl), R⁴⁴-substituted or unsubstituted cycloalkyl (e.g., C₃-C₈ cycloalkyl, C₃-C₆ cycloalkyl, or C₅-C₆ cycloalkyl), R⁴⁴-substituted or unsubstituted heterocycloalkyl (e.g., 3 to 8 membered heterocycloalkyl, 3 to 6 membered heterocycloalkyl, or 5 to 6 membered heterocycloalkyl), R⁴⁴-substituted or unsubstituted aryl (e.g., C₆-C₁₀ aryl, C₁₀ aryl, or phenyl), or R⁴⁴-substituted or unsubstituted heteroaryl (e.g., 5 to 10 membered heteroaryl, 5 to 9 membered heteroaryl, or 5 to 6 membered heteroaryl).

In embodiments, R¹⁰ is R⁴⁴-substituted or unsubstituted alkyl (e.g., C₁-C₈ alkyl, C₁-C₆ alkyl, or C₁-C₄ alkyl). In embodiments, R¹⁰ is R⁴⁴-substituted alkyl (e.g., C₁-C₈ alkyl, C₁-C₆ alkyl, or C₁-C₄ alkyl). In embodiments, R¹⁰ is an unsubstituted alkyl (e.g., C₁-C₈ alkyl, C₁-C₆ alkyl, or C₁-C₄ alkyl). In embodiments, R¹⁰ is R⁴⁴-substituted or unsubstituted heteroalkyl (e.g., 2 to 8 membered heteroalkyl, 2 to 6 membered heteroalkyl, or 2 to 4 membered heteroalkyl). In embodiments, R¹⁰ is R⁴⁴-substituted heteroalkyl (e.g., 2 to 8 membered heteroalkyl, 2 to 6 membered heteroalkyl, or 2 to 4 membered heteroalkyl). In embodiments, R¹⁰ is an unsubstituted heteroalkyl (e.g., 2 to 8 membered heteroalkyl, 2 to 6 membered heteroalkyl, or 2 to 4 membered heteroalkyl). In embodiments, R¹⁰ is R⁴⁴-substituted or unsubstituted cycloalkyl (e.g., C₃-C₈ cycloalkyl, C₃-C₆ cycloalkyl, or C₅-C₆ cycloalkyl). In embodiments, R¹⁰ is R⁴⁴-substituted cycloalkyl (e.g., C₃-C₈ cycloalkyl, C₃-C₆ cycloalkyl, or C₅-C₆ cycloalkyl). In embodiments, R¹⁰ is an unsubstituted cycloalkyl (e.g., C₃-C₈ cycloalkyl, C₃-C₆ cycloalkyl, or C₅-C₆ cycloalkyl). In embodiments, R¹⁰ is R⁴⁴-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹⁰ is R⁴⁴-substituted heterocycloalkyl (e.g., 3 to 8 membered heterocycloalkyl, 3 to 6 membered heterocycloalkyl, or 5 to 6 membered heterocycloalkyl). In embodiments, R¹⁰ is an unsubstituted heterocycloalkyl (e.g., 3 to 8 membered heterocycloalkyl, 3 to 6 membered heterocycloalkyl, or 5 to 6 membered heterocycloalkyl). In embodiments, R¹⁰ is R⁴⁴-substituted or unsubstituted aryl (e.g., C₆-C₁₀ aryl, C₁₀ aryl, or phenyl). In embodiments, R¹⁰ is R⁴⁴-substituted aryl (e.g., C₆-C₁₀ aryl, C₁₀ aryl, or phenyl). In embodiments, R¹⁰ is an unsubstituted aryl (e.g., C₆-C₁₀ aryl, C₁₀ aryl, or phenyl). In embodiments, R¹⁰ is R⁴⁴-substituted or unsubstituted heteroaryl (e.g., 5 to 10 membered heteroaryl, 5 to 9 membered heteroaryl, or 5 to 6 membered heteroaryl). In embodiments, R¹⁰ is R⁴⁴-substituted heteroaryl (e.g., 5 to 10 membered heteroaryl, 5 to 9 membered heteroaryl, or 5 to 6 membered heteroaryl). In embodiments, R¹⁰ is an unsubstituted heteroaryl (e.g., 5 to 10 membered heteroaryl, 5 to 9 membered heteroaryl, or 5 to 6 membered heteroaryl).

R⁴⁴ is independently oxo, halogen, —CCl₃, —CBr₃, —CF₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —CH₂F, —CH₂Cl, —CH₂Br, —CH₂I, R⁴⁵-substituted or unsubstituted alkyl (e.g., C₁-C₈ alkyl, C₁-C₆ alkyl, or C₁-C₄ alkyl), R⁴⁵-substituted or unsubstituted heteroalkyl (e.g., 2 to 8 membered heteroalkyl, 2 to 6 membered heteroalkyl, or 2 to 4 membered heteroalkyl), R⁴⁵-substituted or unsubstituted cycloalkyl (e.g., C₃-C₈ cycloalkyl, C₃-C₆ cycloalkyl, or C₅-C₆ cycloalkyl), R⁴⁵-substituted or unsubstituted heterocycloalkyl (e.g., 3 to 8 membered heterocycloalkyl, 3 to 6 membered heterocycloalkyl, or 5 to 6 membered heterocycloalkyl), R⁴⁵-substituted or unsubstituted aryl (e.g., C₆-C₁₀ aryl, C₁₀ aryl, or phenyl), or R⁴⁵-substituted or unsubstituted heteroaryl (e.g., 5 to 10 membered heteroaryl, 5 to 9 membered heteroaryl, or 5 to 6 membered heteroaryl).

In embodiments, R⁴⁴ is R⁴⁵-substituted or unsubstituted alkyl (e.g., C₁-C₈ alkyl, C₁-C₆ alkyl, or C₁-C₄ alkyl). In embodiments, R⁴⁴ is R⁴⁵-substituted alkyl (e.g., C₁-C₈ alkyl, C₁-C₆ alkyl, or C₁-C₄ alkyl). In embodiments, R⁴⁴ is an unsubstituted alkyl (e.g., C₁-C₈ alkyl, C₁-C₆ alkyl, or C₁-C₄ alkyl). In embodiments, R⁴⁴ is R⁴⁵-substituted or unsubstituted heteroalkyl (e.g., 2 to 8 membered heteroalkyl, 2 to 6 membered heteroalkyl, or 2 to 4 membered heteroalkyl). In embodiments, R⁴⁴ is R⁴⁵-substituted heteroalkyl (e.g., 2 to 8 membered heteroalkyl, 2 to 6 membered heteroalkyl, or 2 to 4 membered heteroalkyl). In embodiments, R⁴⁴ is an unsubstituted heteroalkyl (e.g., 2 to 8 membered heteroalkyl, 2 to 6 membered heteroalkyl, or 2 to 4 membered heteroalkyl). In embodiments, R⁴⁴ is R⁴⁵-substituted or unsubstituted cycloalkyl (e.g., C₃-C₈ cycloalkyl, C₃-C₆ cycloalkyl, or C₅-C₆ cycloalkyl). In embodiments, R⁴⁴ is R⁴⁵-substituted cycloalkyl (e.g., C₃-C₈ cycloalkyl, C₃-C₆ cycloalkyl, or C₅-C₆ cycloalkyl). In embodiments, R⁴⁴ is an unsubstituted cycloalkyl (e.g., C₃-C₈ cycloalkyl, C₃-C₆ cycloalkyl, or C₅-C₆ cycloalkyl). In embodiments, R⁴⁴ is R⁴⁵-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴⁴ is R⁴⁵-substituted heterocycloalkyl (e.g., 3 to 8 membered heterocycloalkyl, 3 to 6 membered heterocycloalkyl, or 5 to 6 membered heterocycloalkyl). In embodiments, R⁴⁴ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁴⁴ is R⁴⁵-substituted or unsubstituted aryl (e.g., C₆-C₁₀ aryl, C₁₀ aryl, or phenyl). In embodiments, R⁴⁴ is R⁴⁵-substituted aryl (e.g., C₆-C₁₀ aryl, C₁₀ aryl, or phenyl). In embodiments, R⁴⁴ is an unsubstituted aryl (e.g., C₆-C₁₀ aryl, C₁₀ aryl, or phenyl). In embodiments, R⁴⁴ is R⁴⁵-substituted or unsubstituted heteroaryl (e.g., 5 to 10 membered heteroaryl, 5 to 9 membered heteroaryl, or 5 to 6 membered heteroaryl). In embodiments, R⁴⁴ is R⁴⁵-substituted heteroaryl (e.g., 5 to 10 membered heteroaryl, 5 to 9 membered heteroaryl, or 5 to 6 membered heteroaryl). In embodiments, R⁴⁴ is an unsubstituted heteroaryl (e.g., 5 to 10 membered heteroaryl, 5 to 9 membered heteroaryl, or 5 to 6 membered heteroaryl).

R⁴⁵ is independently oxo, halogen, —CCl₃, —CBr₃, —CF₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —CH₂F, —CH₂Cl, —CH₂Br, —CH₂I, R⁴⁶-substituted or unsubstituted alkyl (e.g., C₁-C₈ alkyl, C₁-C₆ alkyl, or C₁-C₄ alkyl), R⁴⁶-substituted or unsubstituted heteroalkyl (e.g., 2 to 8 membered heteroalkyl, 2 to 6 membered heteroalkyl, or 2 to 4 membered heteroalkyl), R⁴⁶-substituted or unsubstituted cycloalkyl (e.g., C₃-C₈ cycloalkyl, C₃-C₆ cycloalkyl, or C₅-C₆ cycloalkyl), R⁴⁶-substituted or unsubstituted heterocycloalkyl (e.g., 3 to 8 membered heterocycloalkyl, 3 to 6 membered heterocycloalkyl, or 5 to 6 membered heterocycloalkyl), R⁴⁶-substituted or unsubstituted aryl (e.g., C₆-C₁₀ aryl, C₁₀ aryl, or phenyl), or R⁴⁶-substituted or unsubstituted heteroaryl (e.g., 5 to 10 membered heteroaryl, 5 to 9 membered heteroaryl, or 5 to 6 membered heteroaryl).

In embodiments, R⁴⁵ is R⁴⁶-substituted or unsubstituted alkyl (e.g., C₁-C₈ alkyl, C₁-C₆ alkyl, or C₁-C₄ alkyl). In embodiments, R⁴⁵ is R⁴⁶-substituted alkyl (e.g., C₁-C₈ alkyl, C₁-C₆ alkyl, or C₁-C₄ alkyl). In embodiments, R⁴⁵ is an unsubstituted alkyl (e.g., C₁-C₈ alkyl, C₁-C₆ alkyl, or C₁-C₄ alkyl). In embodiments, R⁴⁵ is R⁴⁶-substituted or unsubstituted heteroalkyl (e.g., 2 to 8 membered heteroalkyl, 2 to 6 membered heteroalkyl, or 2 to 4 membered heteroalkyl). In embodiments, R⁴⁵ is R⁴⁶-substituted heteroalkyl (e.g., 2 to 8 membered heteroalkyl, 2 to 6 membered heteroalkyl, or 2 to 4 membered heteroalkyl). In embodiments, R⁴⁵ is an unsubstituted heteroalkyl (e.g., 2 to 8 membered heteroalkyl, 2 to 6 membered heteroalkyl, or 2 to 4 membered heteroalkyl). In embodiments, R⁴⁵ is R⁴⁶-substituted or unsubstituted cycloalkyl (e.g., C₃-C₈ cycloalkyl, C₃-C₆ cycloalkyl, or C₅-C₆ cycloalkyl). In embodiments, R⁴⁵ is R⁴⁶-substituted cycloalkyl (e.g., C₃-C₈ cycloalkyl, C₃-C₆ cycloalkyl, or C₅-C₆ cycloalkyl). In embodiments, R⁴⁵ is an unsubstituted cycloalkyl (e.g., C₃-C₈ cycloalkyl, C₃-C₆ cycloalkyl, or C₅-C₆ cycloalkyl). In embodiments, R⁴⁵ is R⁴⁶-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴⁵ is R⁴⁶-substituted heterocycloalkyl (e.g., 3 to 8 membered heterocycloalkyl, 3 to 6 membered heterocycloalkyl, or 5 to 6 membered heterocycloalkyl). In embodiments, R⁴⁵ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁴⁵ is R⁴⁶-substituted or unsubstituted aryl (e.g., C₆-C₁₀ aryl, C₁₀ aryl, or phenyl). In embodiments, R⁴⁵ is R⁴⁶-substituted aryl (e.g., C₆-C₁₀ aryl, C₁₀ aryl, or phenyl). In embodiments, R⁴⁵ is an unsubstituted aryl (e.g., C₆-C₁₀ aryl, C₁₀ aryl, or phenyl). In embodiments, R⁴⁵ is R⁴⁶-substituted or unsubstituted heteroaryl (e.g., 5 to 10 membered heteroaryl, 5 to 9 membered heteroaryl, or 5 to 6 membered heteroaryl). In embodiments, R⁴⁵ is R⁴⁶-substituted heteroaryl (e.g., 5 to 10 membered heteroaryl, 5 to 9 membered heteroaryl, or 5 to 6 membered heteroaryl). In embodiments, R⁴⁵ is an unsubstituted heteroaryl (e.g., 5 to 10 membered heteroaryl, 5 to 9 membered heteroaryl, or 5 to 6 membered heteroaryl).

In embodiments, R^{10A}, R^{10B}, R^{10C} and R^{10D} are independently hydrogen, halogen, —CF₃, —CCl₃, —CBr₃, —CI₃, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). R^{10B} and R^{10C} substituents bonded to the same nitrogen atom may optionally be joined to form a substituted or unsubstituted heterocycloalkyl or substituted or unsubstituted heteroaryl.

In embodiments, R^{10A} is hydrogen, halogen, —CF₃, —CCl₃, —CBr₃, —CI₃, —COOH, —CONH₂, R⁴⁴-substituted or unsubstituted alkyl (e.g., C₁-C₈ alkyl, C₁-C₆ alkyl, or C₁-C₄ alkyl), R⁴⁴-substituted or unsubstituted heteroalkyl (e.g., 2 to 8 membered heteroalkyl, 2 to 6 membered heteroalkyl, or 2 to 4 membered heteroalkyl), R⁴⁴-substituted or unsubstituted cycloalkyl (e.g., C₃-C₈ cycloalkyl, C₃-C₆ cycloalkyl, or C₅-C₆ cycloalkyl), R⁴⁴-substituted or unsubstituted heterocycloalkyl (e.g., 3 to 8 membered heterocycloalkyl, 3 to 6 membered heterocycloalkyl, or 5 to 6 membered heterocycloalkyl), R⁴⁴-substituted or unsubstituted aryl (e.g., C₆-C₁₀ aryl, C₁₀ aryl, or phenyl), or R⁴⁴-substituted or unsubstituted heteroaryl (e.g., 5 to 10 membered heteroaryl, 5 to 9 membered heteroaryl, or 5 to 6 membered heteroaryl).

In embodiments, R^{10B} is hydrogen, halogen, —CF₃, —CCl₃, —CBr₃, —CI₃, —COOH, —CONH₂, R⁴⁴-substituted or unsubstituted alkyl (e.g., C₁-C₈ alkyl, C₁-C₆ alkyl, or C₁-C₄ alkyl), R⁴⁴-substituted or unsubstituted heteroalkyl (e.g., 2 to 8 membered heteroalkyl, 2 to 6 membered heteroalkyl, or 2 to 4 membered heteroalkyl), R⁴⁴-substituted or unsubstituted cycloalkyl (e.g., C₃-C₈ cycloalkyl, C₃-C₆ cycloalkyl, or C₅-C₆ cycloalkyl), R⁴⁴-substituted or unsubstituted heterocycloalkyl (e.g., 3 to 8 membered heterocycloalkyl, 3 to 6 membered heterocycloalkyl, or 5 to 6 membered heterocycloalkyl), R⁴⁴-substituted or unsubstituted aryl (e.g., C₆-C₁₀ aryl, C₁₀ aryl, or phenyl), or R⁴⁴-substituted or unsubstituted heteroaryl (e.g., 5 to 10 membered heteroaryl, 5 to 9 membered heteroaryl, or 5 to 6 membered heteroaryl). In embodiments, R^{10B} and R^{10C} substituents bonded to the same nitrogen atom may optionally be joined to form a R⁴⁴⁻substituted or unsubstituted heterocycloalkyl (e.g., 3 to 8 membered heterocycloalkyl, 3 to 6 membered heterocycloalkyl, or 5 to 6 membered heterocycloalkyl) or R⁴⁴-substituted or unsubstituted heteroaryl (e.g., 5 to 10 membered heteroaryl, 5 to 9 membered heteroaryl, or 5 to 6 membered heteroaryl).

In embodiments, R^{10C} is hydrogen, halogen, —CF₃, —CCl₃, —CBr₃, —CI₃, —COOH, —CONH₂, R⁴⁴-substituted or unsubstituted alkyl (e.g., C₁-C₈ alkyl, C₁-C₆ alkyl, or C₁-C₄ alkyl), R⁴⁴-substituted or unsubstituted heteroalkyl (e.g., 2 to 8 membered heteroalkyl, 2 to 6 membered heteroalkyl, or 2 to 4 membered heteroalkyl), R⁴⁴-substituted or unsubstituted cycloalkyl (e.g., C₃-C₈ cycloalkyl, C₃-C₆ cycloalkyl, or C₅-C₆ cycloalkyl), R⁴⁴-substituted or unsubstituted heterocycloalkyl (e.g., 3 to 8 membered heterocycloalkyl, 3 to 6 membered heterocycloalkyl, or 5 to 6 membered heterocycloalkyl), R⁴⁴-substituted or unsubstituted aryl (e.g., C₆-C₁₀ aryl, C₁₀ aryl, or phenyl), or R⁴⁴-substituted or unsubstituted heteroaryl (e.g., 5 to 10 membered heteroaryl, 5 to 9 membered heteroaryl, or 5 to 6 membered heteroaryl). In embodiments, R^{10B} and R^{10C} substituents bonded to the same nitrogen atom may optionally be joined to form a R⁴⁴⁻substituted or unsubstituted heterocycloalkyl (e.g., 3 to 8 membered heterocycloalkyl, 3 to 6 membered heterocycloalkyl, or 5 to 6 membered heterocycloalkyl) or R⁴⁴-substituted or unsubstituted heteroaryl (e.g., 5 to 10 membered heteroaryl, 5 to 9 membered heteroaryl, or 5 to 6 membered heteroaryl).

In embodiments, R^{10D} is hydrogen, halogen, —CF₃, —CCl₃, —CBr₃, —CI₃, —COOH, —CONH₂, R⁴⁴-substituted or unsubstituted alkyl (e.g., C₁-C₈ alkyl, C₁-C₆ alkyl, or C₁-C₄ alkyl), R⁴⁴-substituted or unsubstituted heteroalkyl (e.g., 2 to 8 membered heteroalkyl, 2 to 6 membered heteroalkyl, or 2 to 4 membered heteroalkyl), R⁴⁴-substituted or unsubstituted cycloalkyl (e.g., C₃-C₈ cycloalkyl, C₃-C₆ cycloalkyl, or C₅-C₆ cycloalkyl), R⁴⁴-substituted or unsubstituted heterocycloalkyl (e.g., 3 to 8 membered heterocycloalkyl, 3 to 6 membered heterocycloalkyl, or 5 to 6 membered heterocycloalkyl), R⁴⁴-substituted or unsubstituted aryl (e.g., C₆-C₁₀ aryl, C₁₀ aryl, or phenyl), or R⁴⁴-substituted or unsubstituted heteroaryl (e.g., 5 to 10 membered heteroaryl, 5 to 9 membered heteroaryl, or 5 to 6 membered heteroaryl).

In embodiments, R¹² is hydrogen. In embodiments, R¹² is —N(CH₃)₂ or —NHCH₃. In embodiments, R¹² is —N(CH₃)₂. In embodiments, R¹² and R¹³ are hydrogen. In embodiments, R¹² is hydrogen. In embodiments, R¹² is halogen. In embodiments, R¹² is —CF₃. In embodiments, R¹² is —CCl₃. In embodiments, R¹² is —CBr₃. In embodiments, R¹² is —Cl₃. In embodiments, R¹² is —CHF₂. In embodiments, R¹² is —CHCl₂. In embodiments, R¹² is —CHBr₂. In embodiments, R¹² is —CHI₂. In embodiments, R¹² is —CH₂F. In embodiments, R¹² is —CH₂Cl. In embodiments, R¹² is —CH₂Br. In embodiments, R¹² is —CH₂I. In embodiments, R¹² is —CN. In embodiments, R¹² is —SOₙ₁₂H. In embodiments, R¹² is —SOᵥ₁₂NH₂, —C(O)H. In embodiments, R¹² is —C(O)OH. In embodiments, R¹² is —C(O)NH₂. In embodiments, R¹² is —OH. In embodiments, R¹² is —OCF₃. In embodiments, R¹² is —OCCl₃. In embodiments, R¹² is —OCBr₃. In embodiments, R¹² is —OCl₃. In embodiments, R¹² is —OCHF₂. In embodiments, R¹² is —OCHCl₂. In embodiments, R¹² is —OCHBr₂. In embodiments, R¹² is —OCHI₂. In embodiments, R¹² is —N(CH₃)₂. In embodiments, R¹² is —NH(CH₃). In embodiments, R¹² is —SO₂CH₃.

In embodiments, when R¹² is attached to nitrogen, R¹² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₂H, —SOᵥ₁₂NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹² is hydrogen. In embodiments, when R¹² is attached to nitrogen, R¹² is hydrogen. In embodiments, when R¹² is attached to nitrogen, R¹² is halogen. In embodiments, when R¹² is attached to nitrogen, R¹² is —CF₃. In embodiments, when R¹² is attached to nitrogen, R¹² is —CCl₃. In embodiments, when R¹² is attached to nitrogen, R¹² is —CBr₃. In embodiments, when R¹² is attached to nitrogen, R¹² is —Cl₃. In embodiments, when R¹² is attached to nitrogen, R¹² is —CHF₂. In embodiments, when R¹² is attached to nitrogen, R¹² is —CHCl₂. In embodiments, when R¹² is attached to nitrogen, R¹² is —CHBr₂. In embodiments, when R¹² is attached to nitrogen, R¹² is —CHI₂. In embodiments, when R¹² is attached to nitrogen, R¹² is —CH₂F. In embodiments, when R¹² is attached to nitrogen, R¹² is —CH₂Cl. In embodiments, when R¹² is attached to nitrogen, R¹² is —CH₂Br. In embodiments, when R¹² is attached to nitrogen, R¹² is —CH₂I. In embodiments, when R¹² is attached to nitrogen, R¹² is —CN. In embodiments, when R¹² is attached to nitrogen, R¹² is —SOₙ₁₂H. In embodiments, when R¹² is attached to nitrogen, R¹² is —SOᵥ₁₂NH₂, —C(O)H. In embodiments, when R¹² is attached to nitrogen, R¹² is —C(O)OH. In embodiments, when R¹² is attached to nitrogen, R¹² is —C(O)NH₂. In embodiments, when R¹² is attached to nitrogen, R¹² is —OH, —OCF₃. In embodiments, when R¹² is attached to nitrogen, R¹² is —OCCl₃. In embodiments, when R¹² is attached to nitrogen, R¹² is —OCBr₃. In embodiments, when R¹² is attached to nitrogen, R¹² is —OCl₃. In embodiments, when R¹² is attached to nitrogen, R¹² is —OCHF₂. In embodiments, when R¹² is attached to nitrogen, R¹² is —OCHCl₂. In embodiments, when R¹² is attached to nitrogen, R¹² is —OCHBr₂. In embodiments, when R¹² is attached to nitrogen, R¹² is —OCHI₂. In embodiments, when R¹² is attached to nitrogen, R¹² is —N(CH₃)₂. In embodiments, when R¹² is attached to nitrogen, R¹² is —NH(CH₃). In embodiments, when R¹² is attached to nitrogen, R¹² is —SO₂CH₃. In embodiments, when R¹² is attached to nitrogen, R¹² is —OCH₂F. In embodiments, when R¹² is attached to nitrogen, R¹² is —OCH₂Cl. In embodiments, when R¹² is attached to nitrogen, R¹² is —OCH₂Br. In embodiments, when R¹² is attached to nitrogen, R¹² is —OCH₂I.

In embodiments, R¹² is a substituted or unsubstituted alkyl (e.g., C₁-C₈ alkyl, C₁-C₆ alkyl, or C₁-C₄ alkyl). In embodiments, R¹² is a substituted alkyl (e.g., C₁-C₈ alkyl, C₁-C₆ alkyl, or C₁-C₄ alkyl). In embodiments, R¹² is an unsubstituted alkyl (e.g., C₁-C₈ alkyl, C₁-C₆ alkyl, or C₁-C₄ alkyl). In embodiments, R¹² is a substituted or unsubstituted heteroalkyl (e.g., 2 to 8 membered heteroalkyl, 2 to 6 membered heteroalkyl, or 2 to 4 membered heteroalkyl). In embodiments, R¹² is a substituted heteroalkyl (e.g., 2 to 8 membered heteroalkyl, 2 to 6 membered heteroalkyl, or 2 to 4 membered heteroalkyl). In embodiments, R¹² is an unsubstituted heteroalkyl (e.g., 2 to 8 membered heteroalkyl, 2 to 6 membered heteroalkyl, or 2 to 4 membered heteroalkyl). In embodiments, R¹² is a substituted or unsubstituted cycloalkyl (e.g., C₃-C₈ cycloalkyl, C₃-C₆ cycloalkyl, or C₅-C₆ cycloalkyl). In embodiments, R¹² is a substituted cycloalkyl (e.g., C₃-C₈ cycloalkyl, C₃-C₆ cycloalkyl, or C₅-C₆ cycloalkyl). In embodiments, R¹² is an unsubstituted cycloalkyl (e.g., C₃-C₈ cycloalkyl, C₃-C₆ cycloalkyl, or C₅-C₆ cycloalkyl). In embodiments, R¹² is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹² is a substituted heterocycloalkyl (e.g., 3 to 8 membered heterocycloalkyl, 3 to 6 membered heterocycloalkyl, or 5 to 6 membered heterocycloalkyl). In embodiments, R¹² is an unsubstituted heterocycloalkyl (e.g., 3 to 8 membered heterocycloalkyl, 3 to 6 membered heterocycloalkyl, or 5 to 6 membered heterocycloalkyl). In embodiments, R¹² is a substituted or unsubstituted aryl (e.g., C₆-C₁₀ aryl, C₁₀ aryl, or phenyl). In embodiments, R¹² is a substituted aryl (e.g., C₆-C₁₀ aryl, C₁₀ aryl, or phenyl). In embodiments, R¹² is an unsubstituted aryl (e.g., C₆-C₁₀ aryl, C₁₀ aryl, or phenyl). In embodiments, R¹² is a substituted or unsubstituted heteroaryl (e.g., 5 to 10 membered heteroaryl, 5 to 9 membered heteroaryl, or 5 to 6 membered heteroaryl). In embodiments, R¹² is a substituted heteroaryl (e.g., 5 to 10 membered heteroaryl, 5 to 9 membered heteroaryl, or 5 to 6 membered heteroaryl). In embodiments, R¹² is an unsubstituted heteroaryl (e.g., 5 to 10 membered heteroaryl, 5 to 9 membered heteroaryl, or 5 to 6 membered heteroaryl).

In embodiments, R¹³ is hydrogen. In embodiments, R¹³ is halogen. In embodiments, R¹³ is —CF₃. In embodiments, R¹³ is —CCl₃. In embodiments, R¹³ is —CBr₃. In embodiments, R¹³ is —Cl₃. In embodiments, R¹³ is —CHF₂. In embodiments, R¹³ is —CHCl₂. In embodiments, R¹³ is —CHBr₂. In embodiments, R¹³ is —CHI₂. In embodiments, R¹³ is —CH₂F. In embodiments, R¹³ is —CH₂Cl. In embodiments, R¹³ is —CH₂Br. In embodiments, R¹³ is —CH₂I. In embodiments, R¹³ is —CN. In embodiments, R¹³ is —SOₙ₁₃H. In embodiments, R¹³ is —SOᵥ₁₃NH₂. In embodiments, R¹³ is —C(O)H. In embodiments, R¹³ is —C(O)OH. In embodiments, R¹³ is —C(O)NH₂. In embodiments, R¹³ is —OH. In embodiments, R¹³ is —OCF₃. In embodiments, R¹³ is —OCCl₃. In embodiments, R¹³ is —OCBr₃. In embodiments, R¹³ is —OCl₃. In embodiments, R¹³ is —OCHF₂. In embodiments, R¹³ is —OCHCl₂. In embodiments, R¹³ is —OCHBr₂. In embodiments, R¹³ is —OCHI₂. In embodiments, R¹³ is —N(CH₃)₂. In embodiments, R¹³ is —NH(CH₃).

In embodiments, when R¹³ is attached to nitrogen, R¹³ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₃H, —SOᵥ₁₃NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹³ is hydrogen. In embodiments, when R¹³ is attached to nitrogen, R¹³ is hydrogen. In embodiments, when R¹³ is attached to nitrogen, R¹³ is halogen. In embodiments, when R¹³ is attached to nitrogen, R¹³ is —CF₃. In embodiments, when R¹³ is attached to nitrogen, R¹³ is —CCl₃. In embodiments, when R¹³ is attached to nitrogen, R¹³ is —CBr₃. In embodiments, when R¹³ is attached to nitrogen, R¹³ is —Cl₃. In embodiments, when R¹³ is attached to nitrogen, R¹³ is —CHF₂. In embodiments, when R¹³ is attached to nitrogen, R¹³ is —CHCl₂. In embodiments, when R¹³ is attached to nitrogen, R¹³ is —CHBr₂. In embodiments, when R¹³ is attached to nitrogen, R¹³ is —CHI₂. In embodiments, when R¹³ is attached to nitrogen, R¹³ is —CH₂F. In embodiments, when R¹³ is attached to nitrogen, R¹³ is —CH₂Cl. In embodiments, when R¹³ is attached to nitrogen, R¹³ is —CH₂Br. In embodiments, when R¹³ is attached to nitrogen, R¹³ is —CH₂I. In embodiments, when R¹³ is attached to nitrogen, R¹³ is —CN. In embodiments, when R¹³ is attached to nitrogen, R¹³ is —SOₙ₁₃H. In embodiments, when R¹³ is attached to nitrogen, R¹³ is —SOᵥ₁₃NH₂. In embodiments, when R¹³ is attached to nitrogen, R¹³ is —C(O)H. In embodiments, when R¹³ is attached to nitrogen, R¹³ is —C(O)OH. In embodiments, when R¹³ is attached to nitrogen, R¹³ is —C(O)NH₂. In embodiments, when R¹³ is attached to nitrogen, R¹³ is —OH, —OCF₃. In embodiments, when R¹³ is attached to nitrogen, R¹³ is —OCCl₃. In embodiments, when R¹³ is attached to nitrogen, R¹³ is —OCBr₃. In embodiments, when R¹³ is attached to nitrogen, R¹³ is —OCl₃. In embodiments, when R¹³ is attached to nitrogen, R¹³ is —OCHF₂. In embodiments, when R¹³ is attached to nitrogen, R¹³ is —OCHCl₂. In embodiments, when R¹³ is attached to nitrogen, R¹³ is —OCHBr₂. In embodiments, when R¹³ is attached to nitrogen, R¹³ is —OCHI₂. In embodiments, when R¹³ is attached to nitrogen, R¹³ is —N(CH₃)₂. In embodiments, when R¹³ is attached to nitrogen, R¹³ is —NH(CH₃). In embodiments, when R¹³ is attached to nitrogen, R¹³ is —OCH₂F. In embodiments, when R¹³ is attached to nitrogen, R¹³ is —OCH₂Cl. In embodiments, when R¹³ is attached to nitrogen, R¹³ is —OCH₂Br. In embodiments, when R¹³ is attached to nitrogen, R¹³ is —OCH₂I.

In embodiments, R¹³ is a substituted or unsubstituted alkyl (e.g., C₁-C₈ alkyl, C₁-C₆ alkyl, or C₁-C₄ alkyl). In embodiments, R¹³ is a substituted alkyl (e.g., C₁-C₈ alkyl, C₁-C₆ alkyl, or C₁-C₄ alkyl). In embodiments, R¹³ is an unsubstituted alkyl (e.g., C₁-C₈ alkyl, C₁-C₆ alkyl, or C₁-C₄ alkyl). In embodiments, R¹³ is a substituted or unsubstituted heteroalkyl (e.g., 2 to 8 membered heteroalkyl, 2 to 6 membered heteroalkyl, or 2 to 4 membered heteroalkyl). In embodiments, R¹³ is a substituted heteroalkyl (e.g., 2 to 8 membered heteroalkyl, 2 to 6 membered heteroalkyl, or 2 to 4 membered heteroalkyl). In embodiments, R¹³ is an unsubstituted heteroalkyl (e.g., 2 to 8 membered heteroalkyl, 2 to 6 membered heteroalkyl, or 2 to 4 membered heteroalkyl). In embodiments, R¹³ is a substituted or unsubstituted cycloalkyl (e.g., C₃-C₈ cycloalkyl, C₃-C₆ cycloalkyl, or C₅-C₆ cycloalkyl). In embodiments, R¹³ is a substituted cycloalkyl (e.g., C₃-C₈ cycloalkyl, C₃-C₆ cycloalkyl, or C₅-C₆ cycloalkyl). In embodiments, R¹³ is an unsubstituted cycloalkyl (e.g., C₃-C₈ cycloalkyl, C₃-C₆ cycloalkyl, or C₅-C₆ cycloalkyl). In embodiments, R¹³ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹³ is a substituted heterocycloalkyl (e.g., 3 to 8 membered heterocycloalkyl, 3 to 6 membered heterocycloalkyl, or 5 to 6 membered heterocycloalkyl). In embodiments, R¹³ is an unsubstituted heterocycloalkyl (e.g., 3 to 8 membered heterocycloalkyl, 3 to 6 membered heterocycloalkyl, or 5 to 6 membered heterocycloalkyl). In embodiments, R¹³ is a substituted or unsubstituted aryl (e.g., C₆-C₁₀ aryl, C₁₀ aryl, or phenyl). In embodiments, R¹³ is a substituted aryl (e.g., C₆-C₁₀ aryl, C₁₀ aryl, or phenyl). In embodiments, R¹³ is an unsubstituted aryl (e.g., C₆-C₁₀ aryl, C₁₀ aryl, or phenyl). In embodiments, R¹³ is a substituted or unsubstituted heteroaryl (e.g., 5 to 10 membered heteroaryl, 5 to 9 membered heteroaryl, or 5 to 6 membered heteroaryl). In embodiments, R¹³ is a substituted heteroaryl (e.g., 5 to 10 membered heteroaryl, 5 to 9 membered heteroaryl, or 5 to 6 membered heteroaryl). In embodiments, R¹³ is an unsubstituted heteroaryl (e.g., 5 to 10 membered heteroaryl, 5 to 9 membered heteroaryl, or 5 to 6 membered heteroaryl).

In embodiments, when R¹³ is attached to nitrogen, R¹³ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₃H, —SOᵥ₁₃NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹³ is hydrogen.

In embodiments, when R¹⁴ is attached to nitrogen, R¹⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO₂CH₃, —SOₙ₁₄H, —SOᵥ₁₄NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁴ is hydrogen.

In embodiments, R¹⁴ and R¹⁵ are hydrogen. In embodiments, R¹⁴ is hydrogen. In embodiments, R¹⁴ is halogen. In embodiments, R¹⁴ is —CF₃. In embodiments, R¹⁴ is —CCl₃. In embodiments, R¹⁴ is —CBr₃. In embodiments, R¹⁴ is —Cl₃. In embodiments, R¹⁴ is —CHF₂. In embodiments, R¹⁴ is —CHCl₂. In embodiments, R¹⁴ is —CHBr₂. In embodiments, R¹⁴ is —CHI₂. In embodiments, R¹⁴ is —CH₂F. In embodiments, R¹⁴ is —CH₂Cl. In embodiments, R¹⁴ is —CH₂Br. In embodiments, R¹⁴ is —CH₂I. In embodiments, R¹⁴ is —CN. In embodiments, R¹⁴ is —SOₙ₁₄H. In embodiments, R¹⁴ is —SOᵥ₁₄NH₂. In embodiments, R¹⁴ is —C(O)H. In embodiments, R¹⁴ is —C(O)OH. In embodiments, R¹⁴ is —C(O)NH₂. In embodiments, R¹⁴ is —OH. In embodiments, R¹⁴ is —OCF₃. In embodiments, R¹⁴ is —OCCl₃. In embodiments, R¹⁴ is —OCBr₃. In embodiments, R¹⁴ is —OCl₃. In embodiments, R¹⁴ is —OCHF₂. In embodiments, R¹⁴ is —OCHCl₂. In embodiments, R¹⁴ is —OCHBr₂. In embodiments, R¹⁴ is —OCHI₂. In embodiments, R¹⁴ is —N(CH₃)₂. In embodiments, R¹⁴ is —NH(CH₃). In embodiments, R¹⁴ is —SO₂CH₃.

In embodiments, when R¹⁴ is attached to nitrogen, R¹⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₄H, —SOᵥ₁₄NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁴ is hydrogen. In embodiments, when R¹⁴ is attached to nitrogen, R¹⁴ is hydrogen. In embodiments, when R¹⁴ is attached to nitrogen, R¹⁴ is halogen. In embodiments, when R¹⁴ is attached to nitrogen, R¹⁴ is —CF₃. In embodiments, when R¹⁴ is attached to nitrogen, R¹⁴ is —CCl₃. In embodiments, when R¹⁴ is attached to nitrogen, R¹⁴ is —CBr₃. In embodiments, when R¹⁴ is attached to nitrogen, R¹⁴ is —Cl₃. In embodiments, when R¹⁴ is attached to nitrogen, R¹⁴ is —CHF₂. In embodiments, when R¹⁴ is attached to nitrogen, R¹⁴ is —CHCl₂. In embodiments, when R¹⁴ is attached to nitrogen, R¹⁴ is —CHBr₂. In embodiments, when R¹⁴ is attached to nitrogen, R¹⁴ is —CHI₂. In embodiments, when R¹⁴ is attached to nitrogen, R¹⁴ is —CH₂F. In embodiments, when R¹⁴ is attached to nitrogen, R¹⁴ is —CH₂Cl. In embodiments, when R¹⁴ is attached to nitrogen, R¹⁴ is —CH₂Br. In embodiments, when R¹⁴ is attached to nitrogen, R¹⁴ is —CH₂I. In embodiments, when R¹⁴ is attached to nitrogen, R¹⁴ is —CN. In embodiments, when R¹⁴ is attached to nitrogen, R¹⁴ is —SOₙ₁₄H. In embodiments, when R¹⁴ is attached to nitrogen, R¹⁴ is —SOᵥ₁₄NH₂. In embodiments, when R¹⁴ is attached to nitrogen, R¹⁴ is —C(O)H. In embodiments, when R¹⁴ is attached to nitrogen, R¹⁴ is —C(O)OH. In embodiments, when R¹⁴ is attached to nitrogen, R¹⁴ is —C(O)NH₂. In embodiments, when R¹⁴ is attached to nitrogen, R¹⁴ is —OH. In embodiments, when R¹⁴ is attached to nitrogen, R¹⁴ is —OCF₃. In embodiments, when R¹⁴ is attached to nitrogen, R¹⁴ is —OCCl₃. In embodiments, when R¹⁴ is attached to nitrogen, R¹⁴ is —OCBr₃. In embodiments, when R¹⁴ is attached to nitrogen, R¹⁴ is —OCl₃. In embodiments, when R¹⁴ is attached to nitrogen, R¹⁴ is —OCHF₂. In embodiments, when R¹⁴ is attached to nitrogen, R¹⁴ is —OCHCl₂. In embodiments, when R¹⁴ is attached to nitrogen, R¹⁴ is —OCHBr₂. In embodiments, when R¹⁴ is attached to nitrogen, R¹⁴ is —OCHI₂. In embodiments, when R¹⁴ is attached to nitrogen, R¹⁴ is —N(CH₃)₂. In embodiments, when R¹⁴ is attached to nitrogen, R¹⁴ is —NH(CH₃). In embodiments, when R¹⁴ is attached to nitrogen, R⁴³ is —OCH₂F. In embodiments, when R¹⁴ is attached to nitrogen, R¹⁴ is —OCH₂Cl. In embodiments, when R¹⁴ is attached to nitrogen, R¹⁴ is —OCH₂Br. In embodiments, when R¹⁴ is attached to nitrogen, R¹⁴ is —OCH₂I.

In embodiments, R¹⁴ is a substituted or unsubstituted alkyl (e.g., C₁-C₈ alkyl, C₁-C₆ alkyl, or C₁-C₄ alkyl). In embodiments, R¹⁴ is a substituted alkyl (e.g., C₁-C₈ alkyl, C₁-C₆ alkyl, or C₁-C₄ alkyl). In embodiments, R¹⁴ is an unsubstituted alkyl (e.g., C₁-C₈ alkyl, C₁-C₆ alkyl, or C₁-C₄ alkyl). In embodiments, R¹⁴ is a substituted or unsubstituted heteroalkyl (e.g., 2 to 8 membered heteroalkyl, 2 to 6 membered heteroalkyl, or 2 to 4 membered heteroalkyl). In embodiments, R¹⁴ is a substituted heteroalkyl (e.g., 2 to 8 membered heteroalkyl, 2 to 6 membered heteroalkyl, or 2 to 4 membered heteroalkyl). In embodiments, R¹⁴ is an unsubstituted heteroalkyl (e.g., 2 to 8 membered heteroalkyl, 2 to 6 membered heteroalkyl, or 2 to 4 membered heteroalkyl). In embodiments, R¹⁴ is a substituted or unsubstituted cycloalkyl (e.g., C₃-C₈ cycloalkyl, C₃-C₆ cycloalkyl, or C₅-C₆ cycloalkyl). In embodiments, R¹⁴ is a substituted cycloalkyl (e.g., C₃-C₈ cycloalkyl, C₃-C₆ cycloalkyl, or C₅-C₆ cycloalkyl). In embodiments, R¹⁴ is an unsubstituted cycloalkyl (e.g., C₃-C₈ cycloalkyl, C₃-C₆ cycloalkyl, or C₅-C₆ cycloalkyl). In embodiments, R¹⁴ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹⁴ is a substituted heterocycloalkyl (e.g., 3 to 8 membered heterocycloalkyl, 3 to 6 membered heterocycloalkyl, or 5 to 6 membered heterocycloalkyl). In embodiments, R¹⁴ is an unsubstituted heterocycloalkyl (e.g., 3 to 8 membered heterocycloalkyl, 3 to 6 membered heterocycloalkyl, or 5 to 6 membered heterocycloalkyl). In embodiments, R¹⁴ is a substituted or unsubstituted aryl (e.g., C₆-C₁₀ aryl, C₁₀ aryl, or phenyl). In embodiments, R¹⁴ is a substituted aryl (e.g., C₆-C₁₀ aryl, C₁₀ aryl, or phenyl). In embodiments, R¹⁴ is an unsubstituted aryl (e.g., C₆-C₁₀ aryl, C₁₀ aryl, or phenyl). In embodiments, R¹⁴ is a substituted or unsubstituted heteroaryl (e.g., 5 to 10 membered heteroaryl, 5 to 9 membered heteroaryl, or 5 to 6 membered heteroaryl). In embodiments, R¹⁴ is a substituted heteroaryl (e.g., 5 to 10 membered heteroaryl, 5 to 9 membered heteroaryl, or 5 to 6 membered heteroaryl). In embodiments, R¹⁴ is an unsubstituted heteroaryl (e.g., 5 to 10 membered heteroaryl, 5 to 9 membered heteroaryl, or 5 to 6 membered heteroaryl).

In embodiments, R¹⁵ is hydrogen. In embodiments, R¹⁵ is halogen. In embodiments, R¹⁵ is —CF₃. In embodiments, R¹⁵ is —CCl₃. In embodiments, R¹⁵ is —CBr₃. In embodiments, R¹⁵ is —Cl₃. In embodiments, R¹⁵ is —CHF₂. In embodiments, R¹⁵ is —CHCl₂. In embodiments, R¹⁵ is —CHBr₂. In embodiments, R¹⁵ is —CHI₂. In embodiments, R¹⁵ is —CH₂F. In embodiments, R¹⁵ is —CH₂Cl. In embodiments, R¹⁵ is —CH₂Br. In embodiments, R¹⁵ is —CH₂I. In embodiments, R¹⁵ is —CN. In embodiments, R¹⁵ is —SOₙ₁₅H. In embodiments, R¹⁵ is —SOᵥ₁₅NH₂. In embodiments, R¹⁵ is —C(O)H. In embodiments, R¹⁵ is —C(O)OH. In embodiments, R¹⁵ is —C(O)NH₂. In embodiments, R¹⁵ is —OH. In embodiments, R¹⁵ is —OCF₃. In embodiments, R¹⁵ is —OCCl₃. In embodiments, R¹⁵ is —OCBr₃. In embodiments, R¹⁵ is —OCl₃. In embodiments, R¹⁵ is —OCHF₂. In embodiments, R¹⁵ is —OCHCl₂. In embodiments, R¹⁵ is —OCHBr₂. In embodiments, R¹⁵ is —OCHI₂. In embodiments, R¹⁵ is —N(CH₃)₂. In embodiments, R¹⁵ is —NH(CH₃).

In embodiments, when R¹⁵ is attached to nitrogen, R¹⁵ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₅H, —SOᵥ₁₅NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁵ is hydrogen. In embodiments, when R¹⁵ is attached to nitrogen, R¹⁵ is hydrogen. In embodiments, when R¹⁵ is attached to nitrogen, R¹⁵ is halogen. In embodiments, when R¹⁵ is attached to nitrogen, R¹⁵ is —CF₃. In embodiments, when R¹⁵ is attached to nitrogen, R¹⁵ is —CCl₃. In embodiments, when R¹⁵ is attached to nitrogen, R¹⁵ is —CBr₃. In embodiments, when R¹⁵ is attached to nitrogen, R¹⁵ is —Cl₃. In embodiments, when R¹⁵ is attached to nitrogen, R¹⁵ is —CHF₂. In embodiments, when R¹⁵ is attached to nitrogen, R¹⁵ is —CHCl₂. In embodiments, when R¹⁵ is attached to nitrogen, R¹⁵ is —CHBr₂. In embodiments, when R¹⁵ is attached to nitrogen, R¹⁵ is —CHI₂. In embodiments, when R¹⁵ is attached to nitrogen, R¹⁵ is —CH₂F. In embodiments, when R¹⁵ is attached to nitrogen, R¹⁵ is —CH₂Cl. In embodiments, when R¹⁵ is attached to nitrogen, R¹⁵ is —CH₂Br. In embodiments, when R¹⁵ is attached to nitrogen, R¹⁵ is —CH₂I. In embodiments, when R¹⁵ is attached to nitrogen, R¹⁵ is —CN. In embodiments, when R¹⁵ is attached to nitrogen, R¹⁵ is —SOₙ₁₅H. In embodiments, when R¹⁵ is attached to nitrogen, R⁵ is —SOᵥ₁₅NH₂. In embodiments, when R⁵ is attached to nitrogen, R¹⁵ is —C(O)H. In embodiments, when R¹⁵ is attached to nitrogen, R¹⁵ is —C(O)OH. In embodiments, when R¹⁵ is attached to nitrogen, R¹⁵ is —C(O)NH₂. In embodiments, when R¹⁵ is attached to nitrogen, R¹⁵ is —OCF₃. In embodiments, when R⁵ is attached to nitrogen, R¹⁵ is —OCCl₃. In embodiments, when R¹⁵ is attached to nitrogen, R⁵ is —OCBr₃. In embodiments, when R¹⁵ is attached to nitrogen, R¹⁵ is —OCl₃. In embodiments, when R¹⁵ is attached to nitrogen, R¹⁵ is —OCHF₂. In embodiments, when R¹⁵ is attached to nitrogen, R¹⁵ is —OCHCl₂. In embodiments, when R¹⁵ is attached to nitrogen, R¹⁵ is —OCHBr₂. In embodiments, when R¹⁵ is attached to nitrogen, R¹⁵ is —OCHI₂. In embodiments, when R¹⁵ is attached to nitrogen, R⁵ is —N(CH₃)₂. In embodiments, when R¹⁵ is attached to nitrogen, R¹⁵ is —NH(CH₃). In embodiments, when R¹⁵ is attached to nitrogen, R⁵ is —OCH₂F. In embodiments, when R⁵ is attached to nitrogen, R¹⁵ is —OCH₂Cl. In embodiments, when R¹⁵ is attached to nitrogen, R⁵ is —OCH₂Br. In embodiments, when R¹⁵ is attached to nitrogen, R¹⁵ is —OCH₂I.

In embodiments, R¹⁵ is a substituted or unsubstituted alkyl (e.g., C₁-C₈ alkyl, C₁-C₆ alkyl, or C₁-C₄ alkyl). In embodiments, R⁵ is a substituted alkyl (e.g., C₁-C₈ alkyl, C₁-C₆ alkyl, or C₁-C₄ alkyl). In embodiments, R¹⁵ is an unsubstituted alkyl (e.g., C₁-C₈ alkyl, C₁-C₆ alkyl, or C₁-C₄ alkyl). In embodiments, R⁵ is a substituted or unsubstituted heteroalkyl (e.g., 2 to 8 membered heteroalkyl, 2 to 6 membered heteroalkyl, or 2 to 4 membered heteroalkyl). In embodiments, R¹⁵ is a substituted heteroalkyl (e.g., 2 to 8 membered heteroalkyl, 2 to 6 membered heteroalkyl, or 2 to 4 membered heteroalkyl). In embodiments, R⁵ is an unsubstituted heteroalkyl (e.g., 2 to 8 membered heteroalkyl, 2 to 6 membered heteroalkyl, or 2 to 4 membered heteroalkyl). In embodiments, R¹⁵ is a substituted or unsubstituted cycloalkyl (e.g., C₃-C₈ cycloalkyl, C₃-C₆ cycloalkyl, or C₅-C₆ cycloalkyl). In embodiments, R⁵ is a substituted cycloalkyl (e.g., C₃-C₈ cycloalkyl, C₃-C₆ cycloalkyl, or C₅-C₆ cycloalkyl). In embodiments, R⁵ is an unsubstituted cycloalkyl (e.g., C₃-C₈ cycloalkyl, C₃-C₆ cycloalkyl, or C₅-C₆ cycloalkyl). In embodiments, R¹⁵ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹⁵ is a substituted heterocycloalkyl (e.g., 3 to 8 membered heterocycloalkyl, 3 to 6 membered heterocycloalkyl, or 5 to 6 membered heterocycloalkyl). In embodiments, R¹⁵ is an unsubstituted heterocycloalkyl (e.g., 3 to 8 membered heterocycloalkyl, 3 to 6 membered heterocycloalkyl, or 5 to 6 membered heterocycloalkyl). In embodiments, R¹⁵ is a substituted or unsubstituted aryl (e.g., C₆-C₁₀ aryl, C₁₀ aryl, or phenyl). In embodiments, R⁵ is a substituted aryl (e.g., C₆-C₁₀ aryl, C₁₀ aryl, or phenyl). In embodiments, R¹⁵ is an unsubstituted aryl (e.g., C₆-C₁₀ aryl, C₁₀ aryl, or phenyl). In embodiments, R¹⁵ is a substituted or unsubstituted heteroaryl (e.g., 5 to 10 membered heteroaryl, 5 to 9 membered heteroaryl, or 5 to 6 membered heteroaryl). In embodiments, R¹⁵ is a substituted heteroaryl (e.g., 5 to 10 membered heteroaryl, 5 to 9 membered heteroaryl, or 5 to 6 membered heteroaryl). In embodiments, R⁵ is an unsubstituted heteroaryl (e.g., 5 to 10 membered heteroaryl, 5 to 9 membered heteroaryl, or 5 to 6 membered heteroaryl).

In embodiments, when R¹⁵ is attached to nitrogen, R⁵ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₅H, —SOᵥ₁₅NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁵ is hydrogen.

In embodiments, R¹⁶ is hydrogen. In embodiments, R¹⁶ is halogen. In embodiments, R¹⁶ is —CF₃. In embodiments, R¹⁶ is —CCl₃. In embodiments, R¹⁶ is —CBr₃. In embodiments, R¹⁶ is —Cl₃. In embodiments, R¹⁶ is —CHF₂. In embodiments, R¹⁶ is —CHCl₂. In embodiments, R¹⁶ is —CHBr₂. In embodiments, R¹⁶ is —CHI₂. In embodiments, R¹⁶ is —CH₂F. In embodiments, R¹⁶ is —CH₂Cl. In embodiments, R¹⁶ is —CH₂Br. In embodiments, R¹⁶ is —CH₂I. In embodiments, R¹⁶ is —CN. In embodiments, R¹⁶ is —SOₙ₁₆H. In embodiments, R¹⁶ is —SOᵥ₁₆NH₂. In embodiments, R¹⁶ is —C(O)H. In embodiments, R¹⁶ is —C(O)OH. In embodiments, R¹⁶ is —C(O)NH₂. In embodiments, R¹⁶ is —OH. In embodiments, R¹⁶ is —OCF₃. In embodiments, R¹⁶ is —OCCl₃. In embodiments, R¹⁶ is —OCBr₃. In embodiments, R¹⁶ is —OCl₃. In embodiments, R¹⁶ is —OCHF₂. In embodiments, R¹⁶ is —OCHCl₂. In embodiments, R¹⁶ is —OCHBr₂. In embodiments, R¹⁶ is —OCHI₂. In embodiments, R¹⁶ is —N(CH₃)₂. In embodiments, R¹⁶ is —NH(CH₃).

In embodiments, when R¹⁶ is attached to nitrogen, R¹⁶ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₆H, —SOᵥ₁₆NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁶ is hydrogen. In embodiments, when R¹⁶ is attached to nitrogen, R¹⁶ is hydrogen. In embodiments, when R¹⁶ is attached to nitrogen, R¹⁶ is halogen. In embodiments, when R¹⁶ is attached to nitrogen, R¹⁶ is —CF₃. In embodiments, when R¹⁶ is attached to nitrogen, R¹⁶ is —CCl₃. In embodiments, when R¹⁶ is attached to nitrogen, R¹⁶ is —CBr₃. In embodiments, when R¹⁶ is attached to nitrogen, R¹⁶ is —Cl₃. In embodiments, when R¹⁶ is attached to nitrogen, R¹⁶ is —CHF₂. In embodiments, when R¹⁶ is attached to nitrogen, R¹⁶ is —CHCl₂. In embodiments, when R¹⁶ is attached to nitrogen, R¹⁶ is —CHBr₂. In embodiments, when R¹⁶ is attached to nitrogen, R¹⁶ is —CHI₂. In embodiments, when R¹⁶ is attached to nitrogen, R¹⁶ is —CH₂F. In embodiments, when R¹⁶ is attached to nitrogen, R¹⁶ is —CH₂Cl. In embodiments, when R¹⁶ is attached to nitrogen, R¹⁶ is —CH₂Br. In embodiments, when R¹⁶ is attached to nitrogen, R¹⁶ is —CH₂I. In embodiments, when R¹⁶ is attached to nitrogen, R¹⁶ is —CN. In embodiments, when R¹⁶ is attached to nitrogen, R¹⁶ is —SOₙ₁₆H. In embodiments, when R¹⁶ is attached to nitrogen, R¹⁶ is —SOᵥ₁₆NH₂. In embodiments, when R¹⁶ is attached to nitrogen, R¹⁶ is —C(O)H. In embodiments, when R¹⁶ is attached to nitrogen, R¹⁶ is —C(O)OH. In embodiments, when R¹⁶ is attached to nitrogen, R¹⁶ is —C(O)NH₂. In embodiments, when R¹⁶ is attached to nitrogen, R¹⁶ is —OH. In embodiments, when R¹⁶ is attached to nitrogen, R¹⁶ is —OCF₃. In embodiments, when R¹⁶ is attached to nitrogen, R¹⁶ is —OCCl₃. In embodiments, when R¹⁶ is attached to nitrogen, R¹⁶ is —OCBr₃. In embodiments, when R¹⁶ is attached to nitrogen, R¹⁶ is —OCl₃. In embodiments, when R¹⁶ is attached to nitrogen, R¹⁶ is —OCHF₂. In embodiments, when R¹⁶ is attached to nitrogen, R¹⁶ is —OCHCl₂. In embodiments, when R¹⁶ is attached to nitrogen, R¹⁶ is —OCHBr₂. In embodiments, when R¹⁶ is attached to nitrogen, R¹⁶ is —OCHI₂. In embodiments, when R¹⁶ is attached to nitrogen, R¹⁶ is —N(CH₃)₂. In embodiments, when R¹⁶ is attached to nitrogen, R¹⁶ is —NH(CH₃). In embodiments, when R¹⁶ is attached to nitrogen, R¹⁶ is —OCH₂F. In embodiments, when R¹⁶ is attached to nitrogen, R¹⁶ is —OCH₂Cl. In embodiments, when R¹⁶ is attached to nitrogen, R¹⁶ is —OCH₂Br. In embodiments, when R¹⁶ is attached to nitrogen, R¹⁶ is —OCH₂I.

In embodiments, R¹⁶ is a substituted or unsubstituted alkyl (e.g., C₁-C₈ alkyl, C₁-C₆ alkyl, or C₁-C₄ alkyl). In embodiments, R¹⁶ is a substituted alkyl (e.g., C₁-C₈ alkyl, C₁-C₆ alkyl, or C₁-C₄ alkyl). In embodiments, R¹⁶ is an unsubstituted alkyl (e.g., C₁-C₈ alkyl, C₁-C₆ alkyl, or C₁-C₄ alkyl). In embodiments, R¹⁶ is a substituted or unsubstituted heteroalkyl (e.g., 2 to 8 membered heteroalkyl, 2 to 6 membered heteroalkyl, or 2 to 4 membered heteroalkyl). In embodiments, R¹⁶ is a substituted heteroalkyl (e.g., 2 to 8 membered heteroalkyl, 2 to 6 membered heteroalkyl, or 2 to 4 membered heteroalkyl). In embodiments, R¹⁶ is an unsubstituted heteroalkyl (e.g., 2 to 8 membered heteroalkyl, 2 to 6 membered heteroalkyl, or 2 to 4 membered heteroalkyl). In embodiments, R¹⁶ is a substituted or unsubstituted cycloalkyl (e.g., C₃-C₈ cycloalkyl, C₃-C₆ cycloalkyl, or C₅-C₆ cycloalkyl). In embodiments, R¹⁶ is a substituted cycloalkyl (e.g., C₃-C₈ cycloalkyl, C₃-C₆ cycloalkyl, or C₅-C₆ cycloalkyl). In embodiments, R¹⁶ is an unsubstituted cycloalkyl (e.g., C₃-C₈ cycloalkyl, C₃-C₆ cycloalkyl, or C₅-C₆ cycloalkyl). In embodiments, R¹⁶ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹⁶ is a substituted heterocycloalkyl (e.g., 3 to 8 membered heterocycloalkyl, 3 to 6 membered heterocycloalkyl, or 5 to 6 membered heterocycloalkyl). In embodiments, R¹⁶ is an unsubstituted heterocycloalkyl (e.g., 3 to 8 membered heterocycloalkyl, 3 to 6 membered heterocycloalkyl, or 5 to 6 membered heterocycloalkyl). In embodiments, R¹⁶ is a substituted or unsubstituted aryl (e.g., C₆-C₁₀ aryl, C₁₀ aryl, or phenyl). In embodiments, R¹⁶ is a substituted aryl (e.g., C₆-C₁₀ aryl, C₁₀ aryl, or phenyl). In embodiments, R¹⁶ is an unsubstituted aryl (e.g., C₆-C₁₀ aryl, C₁₀ aryl, or phenyl). In embodiments, R¹⁶ is a substituted or unsubstituted heteroaryl (e.g., 5 to 10 membered heteroaryl, 5 to 9 membered heteroaryl, or 5 to 6 membered heteroaryl). In embodiments, R¹⁶ is a substituted heteroaryl (e.g., 5 to 10 membered heteroaryl, 5 to 9 membered heteroaryl, or 5 to 6 membered heteroaryl). In embodiments, R¹⁶ is an unsubstituted heteroaryl (e.g., 5 to 10 membered heteroaryl, 5 to 9 membered heteroaryl, or 5 to 6 membered heteroaryl).

In embodiments, when R¹⁶ is attached to nitrogen, R¹⁶ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₆H, —SOᵥ₁₆NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁶ is hydrogen.

In embodiments, R¹⁷ is independently halogen. In embodiments, R⁷ is independently oxo. In embodiments, R¹⁷ is independently —CF₃. In embodiments, R¹⁷ is independently —CCl₃. In embodiments, R¹⁷ is independently —CBr₃. In embodiments, R¹⁷ is independently —Cl₃. In embodiments, R¹⁷ is independently —CHF₂. In embodiments, R¹⁷ is independently —CHCl₂. In embodiments, R¹⁷ is independently —CHBr₂. In embodiments, R¹⁷ is independently —CHI₂. In embodiments, R¹⁷ is independently —CH₂F. In embodiments, R¹⁷ is independently —CH₂Cl. In embodiments, R¹⁷ is independently —CH₂Br. In embodiments, R¹⁷ is independently —CH₂I. In embodiments, R¹⁷ is independently —CN. In embodiments, R¹⁷ is independently —N₃. In embodiments, R¹⁷ is independently —SOₙ₁₇H. In embodiments, R¹⁷ is independently —SOᵥ₁₇NH₂. In embodiments, R¹⁷ is independently —NHNH₂. In embodiments, R¹⁷ is independently —ONH₂. In embodiments, R¹⁷ is independently —NHC(O)NHNH₂. In embodiments, R¹⁷ is independently —NHC(O)NH₂. In embodiments, R¹⁷ is independently —N(O)ₘ₁₇. In embodiments, R¹⁷ is independently —NH₂. In embodiments, R¹⁷ is independently —C(O)H. In embodiments, R¹⁷ is independently —C(O)OH. In embodiments, R¹⁷ is independently —C(O)NH₂. In embodiments, R¹⁷ is independently —OH. In embodiments, R¹⁷ is independently —NHSO₂H. In embodiments, R¹⁷ is independently —NHC(O)H. In embodiments, R¹⁷ is independently —NHC(O)OH. In embodiments, R¹⁷ is independently —NHOH. In embodiments, R¹⁷ is independently —OCF₃. In embodiments, R¹⁷ is independently —OCCl₃. In embodiments, R¹⁷ is independently —OCBr₃. In embodiments, R¹⁷ is independently —OCl₃. In embodiments, R¹⁷ is independently —OCHF₂. In embodiments, R¹⁷ is independently —OCHCl₂. In embodiments, R¹⁷ is independently —OCHBr₂. In embodiments, R¹⁷ is independently —OCHI₂. In embodiments, R¹⁷ is independently —OCH₂F. In embodiments, R¹⁷ is independently —OCH₂Cl. In embodiments, R¹⁷ is independently —OCH₂Br. In embodiments, R¹⁷ is independently —OCH₂I.

In embodiments, R¹⁷ is a substituted or unsubstituted alkyl (e.g., C₁-C₈ alkyl, C₁-C₆ alkyl, or C₁-C₄ alkyl). In embodiments, R¹⁷ is a substituted alkyl (e.g., C₁-C₈ alkyl, C₁-C₆ alkyl, or C₁-C₄ alkyl). In embodiments, R¹⁷ is an unsubstituted alkyl (e.g., C₁-C₈ alkyl, C₁-C₆ alkyl, or C₁-C₄ alkyl). In embodiments, R¹⁷ is a substituted or unsubstituted heteroalkyl (e.g., 2 to 8 membered heteroalkyl, 2 to 6 membered heteroalkyl, or 2 to 4 membered heteroalkyl). In embodiments, R¹⁷ is a substituted heteroalkyl (e.g., 2 to 8 membered heteroalkyl, 2 to 6 membered heteroalkyl, or 2 to 4 membered heteroalkyl). In embodiments, R¹⁷ is an unsubstituted heteroalkyl (e.g., 2 to 8 membered heteroalkyl, 2 to 6 membered heteroalkyl, or 2 to 4 membered heteroalkyl). In embodiments, R¹⁷ is a substituted or unsubstituted cycloalkyl (e.g., C₃-C₈ cycloalkyl, C₃-C₆ cycloalkyl, or C₅-C₆ cycloalkyl). In embodiments, R¹⁷ is a substituted cycloalkyl (e.g., C₃-C₈ cycloalkyl, C₃-C₆ cycloalkyl, or C₅-C₆ cycloalkyl). In embodiments, R¹⁷ is an unsubstituted cycloalkyl (e.g., C₃-C₈ cycloalkyl, C₃-C₆ cycloalkyl, or C₅-C₆ cycloalkyl). In embodiments, R¹⁷ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹⁷ is a substituted heterocycloalkyl (e.g., 3 to 8 membered heterocycloalkyl, 3 to 6 membered heterocycloalkyl, or 5 to 6 membered heterocycloalkyl). In embodiments, R¹⁷ is an unsubstituted heterocycloalkyl (e.g., 3 to 8 membered heterocycloalkyl, 3 to 6 membered heterocycloalkyl, or 5 to 6 membered heterocycloalkyl). In embodiments, R¹⁷ is a substituted or unsubstituted aryl (e.g., C₆-C₁₀ aryl, C₁₀ aryl, or phenyl). In embodiments, R¹⁷ is a substituted aryl (e.g., C₆-C₁₀ aryl, C₁₀ aryl, or phenyl). In embodiments, R¹⁷ is an unsubstituted aryl (e.g., C₆-C₁₀ aryl, C₁₀ aryl, or phenyl). In embodiments, R¹⁷ is a substituted or unsubstituted heteroaryl (e.g., 5 to 10 membered heteroaryl, 5 to 9 membered heteroaryl, or 5 to 6 membered heteroaryl). In embodiments, R¹⁷ is a substituted heteroaryl (e.g., 5 to 10 membered heteroaryl, 5 to 9 membered heteroaryl, or 5 to 6 membered heteroaryl). In embodiments, R¹⁷ is an unsubstituted heteroaryl (e.g., 5 to 10 membered heteroaryl, 5 to 9 membered heteroaryl, or 5 to 6 membered heteroaryl).

In embodiments, R²⁸ is hydrogen. In embodiments, R²⁸ is halogen. In embodiments, R²⁸ is —CF₃. In embodiments, R¹⁸ is —CCl₃. In embodiments, R¹⁸ is —CBr₃. In embodiments, R¹⁸ is —Cl₃. In embodiments, R¹⁸ is —CHF₂. In embodiments, R¹⁸ is —CHCl₂. In embodiments, R¹⁸ is —CHBr₂. In embodiments, R¹⁸ is —CHI₂. In embodiments, R¹⁸ is —CH₂F. In embodiments, R¹⁸ is —CH₂Cl. In embodiments, R¹⁸ is —CH₂Br. In embodiments, R¹⁸ is —CH₂I. In embodiments, R¹⁸ is —CN. In embodiments, R¹⁸ is —SOₙ₁₈H. In embodiments, R¹⁸ is —SOᵥ₁₈NH₂. In embodiments, R¹⁸ is —C(O)H. In embodiments, R¹⁸ is —C(O)OH. In embodiments, R¹⁸ is —C(O)NH₂. In embodiments, R¹⁸ is —OH. In embodiments, R¹⁸ is —OCF₃. In embodiments, R¹⁸ is —OCCl₃. In embodiments, R¹⁸ is —OCBr₃. In embodiments, R¹⁸ is —OCl₃. In embodiments, R¹⁸ is —OCHF₂. In embodiments, R¹⁸ is —OCHCl₂. In embodiments, R¹⁸ is —OCHBr₂. In embodiments, R¹⁸ is —OCHI₂. In embodiments, R¹⁸ is —N(CH₃)₂. In embodiments, R¹⁸ is —NH(CH₃). In embodiments, R¹⁸ is independently —OCH₂F. In embodiments, R¹⁸ is independently —OCH₂Cl. In embodiments, R¹⁸ is independently —OCH₂Br. In embodiments, R¹⁸ is independently —OCH₂I.

In embodiments, when R¹⁸ is attached to nitrogen, R¹⁸ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₈H, —SOᵥ₁₈NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁸ is hydrogen. In embodiments, when R¹⁸ is attached to nitrogen, R¹⁸ is hydrogen. In embodiments, when R¹⁸ is attached to nitrogen, R¹⁸ is halogen. In embodiments, when R¹⁸ is attached to nitrogen, R¹⁸ is —CF₃. In embodiments, when R¹⁸ is attached to nitrogen, R¹⁸ is —CCl₃. In embodiments, when R¹⁸ is attached to nitrogen, R¹⁸ is —CBr₃. In embodiments, when R¹⁸ is attached to nitrogen, R¹⁸ is —Cl₃. In embodiments, when R¹⁸ is attached to nitrogen, R¹⁸ is —CHF₂. In embodiments, when R¹⁸ is attached to nitrogen, R¹⁸ is —CHCl₂. In embodiments, when R¹⁸ is attached to nitrogen, R¹⁸ is —CHBr₂. In embodiments, when R¹⁸ is attached to nitrogen, R¹⁸ is —CHI₂. In embodiments, when R¹⁸ is attached to nitrogen, R¹⁸ is —CH₂F. In embodiments, when R¹⁸ is attached to nitrogen, R¹⁸ is —CH₂Cl. In embodiments, when R¹⁸ is attached to nitrogen, R¹⁸ is —CH₂Br. In embodiments, when R¹⁸ is attached to nitrogen, R¹⁸ is —CH₂I. In embodiments, when R¹⁸ is attached to nitrogen, R¹⁸ is —CN. In embodiments, when R¹⁸ is attached to nitrogen, R¹⁸ is —SOₙ₁₈H. In embodiments, when R¹⁸ is attached to nitrogen, R¹⁸ is —SOᵥ₁₈NH₂. In embodiments, when R¹⁸ is attached to nitrogen, R¹⁸ is —C(O)H. In embodiments, when R¹⁸ is attached to nitrogen, R¹⁸ is —C(O)OH. In embodiments, when R¹⁸ is attached to nitrogen, R¹⁸ is —C(O)NH₂. In embodiments, when R¹⁸ is attached to nitrogen, R¹⁸ is —OH. In embodiments, when R¹⁸ is attached to nitrogen, R¹⁸ is —OCF₃. In embodiments, when R¹⁸ is attached to nitrogen, R¹⁸ is —OCCl₃. In embodiments, when R¹⁸ is attached to nitrogen, R¹⁸ is —OCBr₃. In embodiments, when R¹⁸ is attached to nitrogen, R¹⁸ is —OCl₃. In embodiments, when R¹⁸ is attached to nitrogen, R¹⁸ is —OCHF₂. In embodiments, when R¹⁸ is attached to nitrogen, R¹⁸ is —OCHCl₂. In embodiments, when R¹⁸ is attached to nitrogen, R¹⁸ is —OCHBr₂. In embodiments, when R¹⁸ is attached to nitrogen, R¹⁸ is —OCHI₂. In embodiments, when R¹⁸ is attached to nitrogen, R¹⁸ is —N(CH₃)₂. In embodiments, when R¹⁸ is attached to nitrogen, R¹⁸ is —NH(CH₃). In embodiments, when R¹⁸ is attached to nitrogen, R¹⁸ is —OCH₂F. In embodiments, when R¹⁸ is attached to nitrogen, R¹⁸ is —OCH₂Cl. In embodiments, when R¹⁸ is attached to nitrogen, R¹⁸ is —OCH₂Br. In embodiments, when R¹⁸ is attached to nitrogen, R¹⁸ is —OCH₂I.

In embodiments, R⁸ is a substituted or unsubstituted alkyl (e.g., C₁-C₈ alkyl, C₁-C₆ alkyl, or C₁-C₄ alkyl). In embodiments, R¹⁸ is a substituted alkyl (e.g., C₁-C₈ alkyl, C₁-C₆ alkyl, or C₁-C₄ alkyl). In embodiments, R¹⁸ is an unsubstituted alkyl (e.g., C₁-C₈ alkyl, C₁-C₆ alkyl, or C₁-C₄ alkyl). In embodiments, R¹⁸ is a substituted or unsubstituted heteroalkyl (e.g., 2 to 8 membered heteroalkyl, 2 to 6 membered heteroalkyl, or 2 to 4 membered heteroalkyl). In embodiments, R¹⁸ is a substituted heteroalkyl (e.g., 2 to 8 membered heteroalkyl, 2 to 6 membered heteroalkyl, or 2 to 4 membered heteroalkyl). In embodiments, R¹⁸ is an unsubstituted heteroalkyl (e.g., 2 to 8 membered heteroalkyl, 2 to 6 membered heteroalkyl, or 2 to 4 membered heteroalkyl). In embodiments, R¹⁸ is a substituted or unsubstituted cycloalkyl (e.g., C₃-C₈ cycloalkyl, C₃-C₆ cycloalkyl, or C₅-C₆ cycloalkyl). In embodiments, R¹⁸ is a substituted cycloalkyl (e.g., C₃-C₈ cycloalkyl, C₃-C₆ cycloalkyl, or C₅-C₆ cycloalkyl). In embodiments, R¹⁸ is an unsubstituted cycloalkyl (e.g., C₃-C₈ cycloalkyl, C₃-C₆ cycloalkyl, or C₅-C₆ cycloalkyl). In embodiments, R¹⁸ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹⁸ is a substituted heterocycloalkyl (e.g., 3 to 8 membered heterocycloalkyl, 3 to 6 membered heterocycloalkyl, or 5 to 6 membered heterocycloalkyl). In embodiments, R¹⁸ is an unsubstituted heterocycloalkyl (e.g., 3 to 8 membered heterocycloalkyl, 3 to 6 membered heterocycloalkyl, or 5 to 6 membered heterocycloalkyl). In embodiments, R¹⁸ is a substituted or unsubstituted aryl (e.g., C₆-C₁₀ aryl, C₁₀ aryl, or phenyl). In embodiments, R⁸ is a substituted aryl (e.g., C₆-C₁₀ aryl, C₁₀ aryl, or phenyl). In embodiments, R¹⁸ is an unsubstituted aryl (e.g., C₆-C₁₀ aryl, C₁₀ aryl, or phenyl). In embodiments, R¹⁸ is a substituted or unsubstituted heteroaryl (e.g., 5 to 10 membered heteroaryl, 5 to 9 membered heteroaryl, or 5 to 6 membered heteroaryl). In embodiments, R¹⁸ is a substituted heteroaryl (e.g., 5 to 10 membered heteroaryl, 5 to 9 membered heteroaryl, or 5 to 6 membered heteroaryl). In embodiments, R¹⁸ is an unsubstituted heteroaryl (e.g., 5 to 10 membered heteroaryl, 5 to 9 membered heteroaryl, or 5 to 6 membered heteroaryl).

In embodiments, when R¹⁸ is attached to nitrogen, R⁸ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₈H, —SOᵥ₁₈NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁸ is hydrogen.

In embodiments, R¹⁹ is hydrogen. In embodiments, R¹⁹ is halogen. In embodiments, R¹⁹ is —CF₃. In embodiments, R¹⁹ is —CCl₃. In embodiments, R¹⁹ is —CBr₃. In embodiments, R¹⁹ is —Cl₃. In embodiments, R¹⁹ is —CHF₂. In embodiments, R¹⁹ is —CHCl₂. In embodiments, R¹⁹ is —CHBr₂. In embodiments, R¹⁹ is —CHI₂. In embodiments, R¹⁹ is —CH₂F. In embodiments, R¹⁹ is —CH₂Cl. In embodiments, R¹⁹ is —CH₂Br. In embodiments, R¹⁹ is —CH₂I. In embodiments, R¹⁹ is —CN. In embodiments, R¹⁹ is —SOₙ₁₉H. In embodiments, R¹⁹ is —SOᵥ₁₉NH₂. In embodiments, R¹⁹ is —C(O)H. In embodiments, R¹⁹ is —C(O)OH. In embodiments, R¹⁹ is —C(O)NH₂. In embodiments, R¹⁹ is —OH. In embodiments, R¹⁹ is —OCF₃. In embodiments, R¹⁹ is —OCCl₃. In embodiments, R¹⁹ is —OCBr₃. In embodiments, R¹⁹ is —OCl₃. In embodiments, R¹⁹ is —OCHF₂. In embodiments, R¹⁹ is —OCHCl₂. In embodiments, R¹⁹ is —OCHBr₂. In embodiments, R¹⁹ is —OCHI₂. In embodiments, R¹⁹ is —N(CH₃)₂. In embodiments, R¹⁹ is —NH(CH₃). In embodiments, R¹⁹ is —SO₂CH₃. In embodiments, R¹⁹ is independently —OCH₂F. In embodiments, R¹⁹ is independently —OCH₂Cl. In embodiments, R¹⁹ is independently —OCH₂Br. In embodiments, R¹⁹ is independently —OCH₂I.

In embodiments, when R¹⁹ is attached to nitrogen, R¹⁹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₁₉H, —SOᵥ₁₉NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), —SO₂CH₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁹ is hydrogen. In embodiments, when R¹⁹ is attached to nitrogen, R¹⁹ is hydrogen. In embodiments, when R¹⁹ is attached to nitrogen, R¹⁹ is halogen. In embodiments, when R¹⁹ is attached to nitrogen, R¹⁹ is —CF₃. In embodiments, when R¹⁹ is attached to nitrogen, R¹⁹ is —CCl₃. In embodiments, when R¹⁹ is attached to nitrogen, R¹⁹ is —CBr₃. In embodiments, when R¹⁹ is attached to nitrogen, R¹⁹ is —Cl₃. In embodiments, when R¹⁹ is attached to nitrogen, R¹⁹ is —CHF₂. In embodiments, when R¹⁹ is attached to nitrogen, R¹⁹ is —CHCl₂. In embodiments, when R¹⁹ is attached to nitrogen, R¹⁹ is —CHBr₂. In embodiments, when R¹⁹ is attached to nitrogen, R¹⁹ is —CHI₂. In embodiments, when R¹⁹ is attached to nitrogen, R¹⁹ is —CH₂F. In embodiments, when R¹⁹ is attached to nitrogen, R¹⁹ is —CH₂Cl. In embodiments, when R¹⁹ is attached to nitrogen, R¹⁹ is —CH₂Br. In embodiments, when R¹⁹ is attached to nitrogen, R¹⁹ is —CH₂I. In embodiments, when R¹⁹ is attached to nitrogen, R¹⁹ is —CN. In embodiments, when R¹⁹ is attached to nitrogen, R¹⁹ is —SOₙ₉H. In embodiments, when R¹⁹ is attached to nitrogen, R¹⁹ is —SOᵥ₁₉NH₂. In embodiments, when R¹⁹ is attached to nitrogen, R¹⁹ is —C(O)H. In embodiments, when R¹⁹ is attached to nitrogen, R¹⁹ is —C(O)OH. In embodiments, when R¹⁹ is attached to nitrogen, R¹⁹ is —C(O)NH₂. In embodiments, when R¹⁹ is attached to nitrogen, R¹⁹ is —OH. In embodiments, when R¹⁹ is attached to nitrogen, R¹⁹ is —OCF₃. In embodiments, when R¹⁹ is attached to nitrogen, R¹⁹ is —OCCl₃. In embodiments, when R¹⁹ is attached to nitrogen, R¹⁹ is —OCBr₃. In embodiments, when R¹⁹ is attached to nitrogen, R¹⁹ is —OCl₃. In embodiments, when R¹⁹ is attached to nitrogen, R¹⁹ is —OCHF₂. In embodiments, when R¹⁹ is attached to nitrogen, R¹⁹ is —OCHCl₂. In embodiments, when R¹⁹ is attached to nitrogen, R¹⁹ is —OCHBr₂. In embodiments, when R¹⁹ is attached to nitrogen, R¹⁹ is —OCHI₂. In embodiments, when R¹⁹ is attached to nitrogen, R¹⁹ is —N(CH₃)₂. In embodiments, when R¹⁹ is attached to nitrogen, R¹⁹ is —NH(CH₃). In embodiments, when R¹⁹ is attached to nitrogen, R¹⁹ is —SO₂CH₃. In embodiments, when R¹⁹ is attached to nitrogen, R¹⁹ is —OCH₂F. In embodiments, when R¹⁹ is attached to nitrogen, R¹⁹ is —OCH₂Cl. In embodiments, when R¹⁹ is attached to nitrogen, R¹⁹ is —OCH₂Br. In embodiments, when R¹⁹ is attached to nitrogen, R¹⁹ is —OCH₂I.

In embodiments, R¹⁹ is a substituted or unsubstituted alkyl (e.g., C₁-C₈ alkyl, C₁-C₆ alkyl, or C₁-C₄ alkyl). In embodiments, R¹⁹ is a substituted alkyl (e.g., C₁-C₈ alkyl, C₁-C₆ alkyl, or C₁-C₄ alkyl). In embodiments, R¹⁹ is an unsubstituted alkyl (e.g., C₁-C₈ alkyl, C₁-C₆ alkyl, or C₁-C₄ alkyl). In embodiments, R¹⁹ is a substituted or unsubstituted heteroalkyl (e.g., 2 to 8 membered heteroalkyl, 2 to 6 membered heteroalkyl, or 2 to 4 membered heteroalkyl). In embodiments, R¹⁹ is a substituted heteroalkyl (e.g., 2 to 8 membered heteroalkyl, 2 to 6 membered heteroalkyl, or 2 to 4 membered heteroalkyl). In embodiments, R¹⁹ is an unsubstituted heteroalkyl (e.g., 2 to 8 membered heteroalkyl, 2 to 6 membered heteroalkyl, or 2 to 4 membered heteroalkyl). In embodiments, R¹⁹ is a substituted or unsubstituted cycloalkyl (e.g., C₃-C₈ cycloalkyl, C₃-C₆ cycloalkyl, or C₅-C₆ cycloalkyl). In embodiments, R¹⁹ is a substituted cycloalkyl (e.g., C₃-C₈ cycloalkyl, C₃-C₆ cycloalkyl, or C₅-C₆ cycloalkyl). In embodiments, R¹⁹ is an unsubstituted cycloalkyl (e.g., C₃-C₈ cycloalkyl, C₃-C₆ cycloalkyl, or C₅-C₆ cycloalkyl). In embodiments, R¹⁹ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹⁹ is a substituted heterocycloalkyl (e.g., 3 to 8 membered heterocycloalkyl, 3 to 6 membered heterocycloalkyl, or 5 to 6 membered heterocycloalkyl). In embodiments, R¹⁹ is an unsubstituted heterocycloalkyl (e.g., 3 to 8 membered heterocycloalkyl, 3 to 6 membered heterocycloalkyl, or 5 to 6 membered heterocycloalkyl). In embodiments, R¹⁹ is a substituted or unsubstituted aryl (e.g., C₆-C₁₀ aryl, C₁₀ aryl, or phenyl). In embodiments, R¹⁹ is a substituted aryl (e.g., C₆-C₁₀ aryl, C₁₀ aryl, or phenyl). In embodiments, R¹⁹ is an unsubstituted aryl (e.g., C₆-C₁₀ aryl, C₁₀ aryl, or phenyl). In embodiments, R¹⁹ is a substituted or unsubstituted heteroaryl (e.g., 5 to 10 membered heteroaryl, 5 to 9 membered heteroaryl, or 5 to 6 membered heteroaryl). In embodiments, R¹⁹ is a substituted heteroaryl (e.g., 5 to 10 membered heteroaryl, 5 to 9 membered heteroaryl, or 5 to 6 membered heteroaryl). In embodiments, R¹⁹ is an unsubstituted heteroaryl (e.g., 5 to 10 membered heteroaryl, 5 to 9 membered heteroaryl, or 5 to 6 membered heteroaryl).

In embodiments, when R¹⁹ is attached to nitrogen, R¹⁹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₉H, —SOᵥ₁₉NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), —SO₂CH₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁹ is hydrogen.

In embodiments, R²⁰ is hydrogen. In embodiments, R²⁰ is halogen. In embodiments, R²⁰ is —CF₃. In embodiments, R²⁰ is —CCl₃. In embodiments, R²⁰ is —CBr₃. In embodiments, R²⁰ is —Cl₃. In embodiments, R²⁰ is —CHF₂. In embodiments, R²⁰ is —CHCl₂. In embodiments, R²⁰ is —CHBr₂. In embodiments, R²⁰ is —CHI₂. In embodiments, R²⁰ is —CH₂F. In embodiments, R²⁰ is —CH₂Cl. In embodiments, R²⁰ is —CH₂Br. In embodiments, R²⁰ is —CH₂I. In embodiments, R²⁰ is —CN. In embodiments, R²⁰ is —SOₙ₂₀H. In embodiments, R²⁰ is —SOᵥ₂₀NH₂. In embodiments, R²⁰ is —C(O)H. In embodiments, R²⁰ is —C(O)OH. In embodiments, R²⁰ is —C(O)NH₂. In embodiments, R²⁰ is —OH. In embodiments, R²⁰ is —OCF₃. In embodiments, R²⁰ is —OCCl₃. In embodiments, R²⁰ is —OCBr₃. In embodiments, R²⁰ is —OCl₃. In embodiments, R²⁰ is —OCHF₂. In embodiments, R²⁰ is —OCHCl₂. In embodiments, R²⁰ is —OCHBr₂. In embodiments, R²⁰ is —OCHI₂. In embodiments, R²⁰ is —N(CH₃)₂. In embodiments, R²⁰ is —NH(CH₃). In embodiments, R²⁰ is —SO₂CH₃. In embodiments, R²⁰ is independently —OCH₂F. In embodiments, R²⁰ is independently —OCH₂Cl. In embodiments, R²⁰ is independently —OCH₂Br. In embodiments, R²⁰ is independently —OCH₂I.

In embodiments, when R²⁰ is attached to nitrogen, R²⁰ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₀H, —SOᵥ₂₀NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²⁰ is hydrogen. In embodiments, when R²⁰ is attached to nitrogen, R²⁰ is hydrogen. In embodiments, when R²⁰ is attached to nitrogen, R²⁰ is halogen. In embodiments, when R²⁰ is attached to nitrogen, R²⁰ is —CF₃. In embodiments, when R²⁰ is attached to nitrogen, R²⁰ is —CCl₃. In embodiments, when R²⁰ is attached to nitrogen, R²⁰ is —CBr₃. In embodiments, when R²⁰ is attached to nitrogen, R²⁰ is —Cl₃. In embodiments, when R²⁰ is attached to nitrogen, R²⁰ is —CHF₂. In embodiments, when R²⁰ is attached to nitrogen, R²⁰ is —CHCl₂. In embodiments, when R²⁰ is attached to nitrogen, R²⁰ is —CHBr₂. In embodiments, when R²⁰ is attached to nitrogen, R²⁰ is —CHI₂. In embodiments, when R²⁰ is attached to nitrogen, R²⁰ is —CH₂F. In embodiments, when R²⁰ is attached to nitrogen, R²⁰ is —CH₂Cl. In embodiments, when R²⁰ is attached to nitrogen, R²⁰ is —CH₂Br. In embodiments, when R²⁰ is attached to nitrogen, R²⁰ is —CH₂I. In embodiments, when R²⁰ is attached to nitrogen, R²⁰ is —CN. In embodiments, when R²⁰ is attached to nitrogen, R²⁰ is —SOₙ₂₀H. In embodiments, when R²⁰ is attached to nitrogen, R²⁰ is —SOᵥ₂₀NH₂. In embodiments, when R²⁰ is attached to nitrogen, R²⁰ is —C(O)H. In embodiments, when R²⁰ is attached to nitrogen, R²⁰ is —C(O)OH. In embodiments, when R²⁰ is attached to nitrogen, R²⁰ is —C(O)NH₂. In embodiments, when R²⁰ is attached to nitrogen, R²⁰ is —OH, In embodiments, when R²⁰ is attached to nitrogen, R²⁰ is —OCF₃. In embodiments, when R²⁰ is attached to nitrogen, R²⁰ is —OCCl₃. In embodiments, when R²⁰ is attached to nitrogen, R²⁰ is —OCBr₃. In embodiments, when R²⁰ is attached to nitrogen, R²⁰ is —OCl₃. In embodiments, when R²⁰ is attached to nitrogen, R²⁰ is —OCHF₂. In embodiments, when R²⁰ is attached to nitrogen, R²⁰ is —OCHCl₂. In embodiments, when R²⁰ is attached to nitrogen, R²⁰ is —OCHBr₂. In embodiments, when R²⁰ is attached to nitrogen, R²⁰ is —OCHI₂. In embodiments, when R²⁰ is attached to nitrogen, R²⁰ is —N(CH₃)₂. In embodiments, when R²⁰ is attached to nitrogen, R²⁰ is —NH(CH₃). In embodiments, when R²⁰ is attached to nitrogen, R²⁰ is —OCH₂F. In embodiments, when R²⁰ is attached to nitrogen, R²⁰ is —OCH₂Cl. In embodiments, when R²⁰ is attached to nitrogen, R²⁰ is —OCH₂Br. In embodiments, when R²⁰ is attached to nitrogen, R²⁰ is —OCH₂I.

In embodiments, R²⁰ is a substituted or unsubstituted alkyl (e.g., C₁-C₈ alkyl, C₁-C₆ alkyl, or C₁-C₄ alkyl). In embodiments, R²⁰ is a substituted alkyl (e.g., C₁-C₈ alkyl, C₁-C₆ alkyl, or C₁-C₄ alkyl). In embodiments, R²⁰ is an unsubstituted alkyl (e.g., C₁-C₈ alkyl, C₁-C₆ alkyl, or C₁-C₄ alkyl). In embodiments, R²⁰ is a substituted or unsubstituted heteroalkyl (e.g., 2 to 8 membered heteroalkyl, 2 to 6 membered heteroalkyl, or 2 to 4 membered heteroalkyl). In embodiments, R²⁰ is a substituted heteroalkyl (e.g., 2 to 8 membered heteroalkyl, 2 to 6 membered heteroalkyl, or 2 to 4 membered heteroalkyl). In embodiments, R²⁰ is an unsubstituted heteroalkyl (e.g., 2 to 8 membered heteroalkyl, 2 to 6 membered heteroalkyl, or 2 to 4 membered heteroalkyl). In embodiments, R²⁰ is a substituted or unsubstituted cycloalkyl (e.g., C₃-C₈ cycloalkyl, C₃-C₆ cycloalkyl, or C₅-C₆ cycloalkyl). In embodiments, R²⁰ is a substituted cycloalkyl (e.g., C₃-C₈ cycloalkyl, C₃-C₆ cycloalkyl, or C₅-C₆ cycloalkyl). In embodiments, R²⁰ is an unsubstituted cycloalkyl (e.g., C₃-C₈ cycloalkyl, C₃-C₆ cycloalkyl, or C₅-C₆ cycloalkyl). In embodiments, R²⁰ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R²⁰ is a substituted heterocycloalkyl (e.g., 3 to 8 membered heterocycloalkyl, 3 to 6 membered heterocycloalkyl, or 5 to 6 membered heterocycloalkyl). In embodiments, R²⁰ is an unsubstituted heterocycloalkyl (e.g., 3 to 8 membered heterocycloalkyl, 3 to 6 membered heterocycloalkyl, or 5 to 6 membered heterocycloalkyl). In embodiments, R²⁰ is a substituted or unsubstituted aryl (e.g., C₆-C₁₀ aryl, C₁₀ aryl, or phenyl). In embodiments, R²⁰ is a substituted aryl (e.g., C₆-C₁₀ aryl, C₁₀ aryl, or phenyl). In embodiments, R²⁰ is an unsubstituted aryl (e.g., C₆-C₁₀ aryl, C₁₀ aryl, or phenyl). In embodiments, R²⁰ is a substituted or unsubstituted heteroaryl (e.g., 5 to 10 membered heteroaryl, 5 to 9 membered heteroaryl, or 5 to 6 membered heteroaryl). In embodiments, R²⁰ is a substituted heteroaryl (e.g., 5 to 10 membered heteroaryl, 5 to 9 membered heteroaryl, or 5 to 6 membered heteroaryl). In embodiments, R²⁰ is an unsubstituted heteroaryl (e.g., 5 to 10 membered heteroaryl, 5 to 9 membered heteroaryl, or 5 to 6 membered heteroaryl).

In embodiments, when R²⁰ is attached to nitrogen, R²⁰ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₀H, —SOᵥ₂₀NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²⁰ is hydrogen.

In embodiments, R²¹ is hydrogen. In embodiments, R²¹ is halogen. In embodiments, R²¹ is —CF₃. In embodiments, R²¹ is —CCl₃. In embodiments, R²¹ is —CBr₃. In embodiments, R²¹ is —Cl₃. In embodiments, R²¹ is —CHF₂. In embodiments, R²¹ is —CHCl₂. In embodiments, R²¹ is —CHBr₂. In embodiments, R²¹ is —CHI₂. In embodiments, R²¹ is —CH₂F. In embodiments, R²¹ is —CH₂Cl. In embodiments, R²¹ is —CH₂Br. In embodiments, R²¹ is —CH₂I. In embodiments, R²¹ is —CN. In embodiments, R²¹ is —SOₙ₂₁H. In embodiments, R²¹ is —SOᵥ₂₁NH₂. In embodiments, R²¹ is —C(O)H. In embodiments, R²¹ is —C(O)OH. In embodiments, R²¹ is —C(O)NH₂. In embodiments, R²¹ is —OH. In embodiments, R²¹ is —OCF₃. In embodiments, R²¹ is —OCCl₃. In embodiments, R²¹ is —OCBr₃. In embodiments, R²¹ is —OCl₃. In embodiments, R²¹ is —OCHF₂. In embodiments, R²¹ is —OCHCl₂. In embodiments, R²¹ is —OCHBr₂. In embodiments, R²¹ is —OCHI₂. In embodiments, R²¹ is —N(CH₃)₂. In embodiments, R²¹ is —NH(CH₃). In embodiments, R²¹ is —SO₂CH₃. In embodiments, R²¹ is independently —OCH₂F. In embodiments, R²¹ is independently —OCH₂Cl. In embodiments, R²¹ is independently —OCH₂Br. In embodiments, R²¹ is independently —OCH₂I.

In embodiments, when R²¹ is attached to nitrogen, R²¹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₁H, —SOᵥ₂₁NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²¹ is hydrogen. In embodiments, when R²¹ is attached to nitrogen, R²¹ is hydrogen. In embodiments, when R²¹ is attached to nitrogen, R²¹ is halogen. In embodiments, when R²¹ is attached to nitrogen, R²¹ is —CF₃. In embodiments, when R²¹ is attached to nitrogen, R²¹ is —CCl₃. In embodiments, when R²¹ is attached to nitrogen, R²¹ is —CBr₃. In embodiments, when R²¹ is attached to nitrogen, R²¹ is —Cl₃. In embodiments, when R²¹ is attached to nitrogen, R²¹ is —CHF₂. In embodiments, when R²¹ is attached to nitrogen, R²¹ is —CHCl₂. In embodiments, when R²¹ is attached to nitrogen, R²¹ is —CHBr₂. In embodiments, when R²¹ is attached to nitrogen, R²¹ is —CHI₂. In embodiments, when R²¹ is attached to nitrogen, R²¹ is —CH₂F. In embodiments, when R²¹ is attached to nitrogen, R²¹ is —CH₂Cl. In embodiments, when R²¹ is attached to nitrogen, R²¹ is —CH₂Br. In embodiments, when R²¹ is attached to nitrogen, R²¹ is —CH₂I. In embodiments, when R²¹ is attached to nitrogen, R²¹ is —CN. In embodiments, when R²¹ is attached to nitrogen, R²¹ is —SOₙ₂₁H. In embodiments, when R²¹ is attached to nitrogen, R²¹ is —SOᵥ₂₁NH₂. In embodiments, when R²¹ is attached to nitrogen, R²¹ is —C(O)H. In embodiments, when R²¹ is attached to nitrogen, R²¹ is —C(O)OH. In embodiments, when R²¹ is attached to nitrogen, R²¹ is —C(O)NH₂. In embodiments, when R²¹ is attached to nitrogen, R²¹ is —OH. In embodiments, when R²¹ is attached to nitrogen, R²¹ is —OCF₃. In embodiments, when R²¹ is attached to nitrogen, R²¹ is —OCCl₃. In embodiments, when R²¹ is attached to nitrogen, R²¹ is —OCBr₃. In embodiments, when R²¹ is attached to nitrogen, R²¹ is —OCl₃. In embodiments, when R²¹ is attached to nitrogen, R²¹ is —OCHF₂. In embodiments, when R²¹ is attached to nitrogen, R²¹ is —OCHCl₂. In embodiments, when R²¹ is attached to nitrogen, R²¹ is —OCHBr₂. In embodiments, when R²¹ is attached to nitrogen, R²¹ is —OCHI₂. In embodiments, when R²¹ is attached to nitrogen, R²¹ is —N(CH₃)₂. In embodiments, when R²¹ is attached to nitrogen, R²¹ is —NH(CH₃). In embodiments, when R²¹ is attached to nitrogen, R²¹ is —OCH₂F. In embodiments, when R²¹ is attached to nitrogen, R²¹ is —OCH₂Cl. In embodiments, when R²¹ is attached to nitrogen, R²¹ is —OCH₂Br. In embodiments, when R²¹ is attached to nitrogen, R²¹ is —OCH₂I.

In embodiments, R²¹ is a substituted or unsubstituted alkyl (e.g., C₁-C₈ alkyl, C₁-C₆ alkyl, or C₁-C₄ alkyl). In embodiments, R²¹ is a substituted alkyl (e.g., C₁-C₈ alkyl, C₁-C₆ alkyl, or C₁-C₄ alkyl). In embodiments, R²¹ is an unsubstituted alkyl (e.g., C₁-C₈ alkyl, C₁-C₆ alkyl, or C₁-C₄ alkyl). In embodiments, R²¹ is a substituted or unsubstituted heteroalkyl (e.g., 2 to 8 membered heteroalkyl, 2 to 6 membered heteroalkyl, or 2 to 4 membered heteroalkyl). In embodiments, R²¹ is a substituted heteroalkyl (e.g., 2 to 8 membered heteroalkyl, 2 to 6 membered heteroalkyl, or 2 to 4 membered heteroalkyl). In embodiments, R²¹ is an unsubstituted heteroalkyl (e.g., 2 to 8 membered heteroalkyl, 2 to 6 membered heteroalkyl, or 2 to 4 membered heteroalkyl). In embodiments, R²¹ is a substituted or unsubstituted cycloalkyl (e.g., C₃-C₈ cycloalkyl, C₃-C₆ cycloalkyl, or C₅-C₆ cycloalkyl). In embodiments, R²¹ is a substituted cycloalkyl (e.g., C₃-C₈ cycloalkyl, C₃-C₆ cycloalkyl, or C₅-C₆ cycloalkyl). In embodiments, R²¹ is an unsubstituted cycloalkyl (e.g., C₃-C₈ cycloalkyl, C₃-C₆ cycloalkyl, or C₅-C₆ cycloalkyl). In embodiments, R²¹ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R²¹ is a substituted heterocycloalkyl (e.g., 3 to 8 membered heterocycloalkyl, 3 to 6 membered heterocycloalkyl, or 5 to 6 membered heterocycloalkyl). In embodiments, R²¹ is an unsubstituted heterocycloalkyl (e.g., 3 to 8 membered heterocycloalkyl, 3 to 6 membered heterocycloalkyl, or 5 to 6 membered heterocycloalkyl). In embodiments, R²¹ is a substituted or unsubstituted aryl (e.g., C₆-C₁₀ aryl, C₁₀ aryl, or phenyl). In embodiments, R²¹ is a substituted aryl (e.g., C₆-C₁₀ aryl, C₁₀ aryl, or phenyl). In embodiments, R²¹ is an unsubstituted aryl (e.g., C₆-C₁₀ aryl, C₁₀ aryl, or phenyl). In embodiments, R²¹ is a substituted or unsubstituted heteroaryl (e.g., 5 to 10 membered heteroaryl, 5 to 9 membered heteroaryl, or 5 to 6 membered heteroaryl). In embodiments, R²¹ is a substituted heteroaryl (e.g., 5 to 10 membered heteroaryl, 5 to 9 membered heteroaryl, or 5 to 6 membered heteroaryl). In embodiments, R²¹ is an unsubstituted heteroaryl (e.g., 5 to 10 membered heteroaryl, 5 to 9 membered heteroaryl, or 5 to 6 membered heteroaryl).

In embodiments, when R²¹ is attached to nitrogen, R²¹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₁H, —SOᵥ₂₁NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²¹ is hydrogen.

In embodiments, R²² is hydrogen. In embodiments, R²² is halogen. In embodiments, R²² is —CF₃. In embodiments, R²² is —CCl₃. In embodiments, R²² is —CBr₃. In embodiments, R²² is —Cl₃. In embodiments, R²² is —CHF₂. In embodiments, R²² is —CHCl₂. In embodiments, R²² is —CHBr₂. In embodiments, R²² is —CHI₂. In embodiments, R²² is —CH₂F. In embodiments, R²² is —CH₂Cl. In embodiments, R²² is —CH₂Br. In embodiments, R²² is —CH₂I. In embodiments, R²² is —CN. In embodiments, R²² is —SOₙ₂₂H. In embodiments, R²² is —SOᵥ₂₂NH₂. In embodiments, R²² is —C(O)H. In embodiments, R²² is —C(O)OH. In embodiments, R²² is —C(O)NH₂. In embodiments, R²² is —OH. In embodiments, R²² is —OCF₃. In embodiments, R²² is —OCCl₃. In embodiments, R²² is —OCBr₃. In embodiments, R²² is —OCl₃. In embodiments, R²² is —OCHF₂. In embodiments, R²² is —OCHCl₂. In embodiments, R²² is —OCHBr₂. In embodiments, R²² is —OCHI₂. In embodiments, R²² is —N(CH₃)₂. In embodiments, R²² is —NH(CH₃). In embodiments, R²² is independently —OCH₂F. In embodiments, R²² is independently —OCH₂Cl. In embodiments, R²² is independently —OCH₂Br. In embodiments, R²² is independently —OCH₂I.

In embodiments, when R²² is attached to nitrogen, R²² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₂H, —SOᵥ₂₂NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²² is hydrogen. In embodiments, when R²² is attached to nitrogen, R²² is hydrogen. In embodiments, when R²² is attached to nitrogen, R²² is halogen. In embodiments, when R²² is attached to nitrogen, R²² is —CF₃. In embodiments, when R²² is attached to nitrogen, R²² is —CCl₃. In embodiments, when R²² is attached to nitrogen, R²² is —CBr₃. In embodiments, when R²² is attached to nitrogen, R²² is —Cl₃. In embodiments, when R²² is attached to nitrogen, R²² is —CHF₂. In embodiments, when R²² is attached to nitrogen, R²² is —CHCl₂. In embodiments, when R²² is attached to nitrogen, R²² is —CHBr₂. In embodiments, when R²² is attached to nitrogen, R²² is —CHI₂. In embodiments, when R²² is attached to nitrogen, R²² is —CH₂F. In embodiments, when R²² is attached to nitrogen, R²² is —CH₂Cl. In embodiments, when R²² is attached to nitrogen, R²² is —CH₂Br. In embodiments, when R²² is attached to nitrogen, R²² is —CH₂I. In embodiments, when R²² is attached to nitrogen, R²² is —CN. In embodiments, when R²² is attached to nitrogen, R²² is —SOₙ₂₂H. In embodiments, when R²² is attached to nitrogen, R²² is —SOᵥ₂₂NH₂. In embodiments, when R²² is attached to nitrogen, R²² is —C(O)H. In embodiments, when R²² is attached to nitrogen, R²² is —C(O)OH. In embodiments, when R²² is attached to nitrogen, R²² is —C(O)NH₂. In embodiments, when R²² is attached to nitrogen, R²² is —OH. In embodiments, when R²² is attached to nitrogen, R²² is —OCF₃. In embodiments, when R²² is attached to nitrogen, R²² is —OCCl₃. In embodiments, when R²² is attached to nitrogen, R²² is —OCBr₃. In embodiments, when R²² is attached to nitrogen, R²² is —OCl₃. In embodiments, when R²² is attached to nitrogen, R²² is —OCHF₂. In embodiments, when R²² is attached to nitrogen, R²² is —OCHCl₂. In embodiments, when R²² is attached to nitrogen, R²² is —OCHBr₂. In embodiments, when R²² is attached to nitrogen, R²² is —OCHI₂. In embodiments, when R²² is attached to nitrogen, R²² is —N(CH₃)₂. In embodiments, when R²² is attached to nitrogen, R²² is —NH(CH₃). In embodiments, when R²² is attached to nitrogen, R²² is —OCH₂F. In embodiments, when R²² is attached to nitrogen, R²² is —OCH₂Cl. In embodiments, when R²² is attached to nitrogen, R²² is —OCH₂Br. In embodiments, when R²² is attached to nitrogen, R²² is —OCH₂I.

In embodiments, R²² is a substituted or unsubstituted alkyl (e.g., C₁-C₈ alkyl, C₁-C₆ alkyl, or C₁-C₄ alkyl). In embodiments, R²² is a substituted alkyl (e.g., C₁-C₈ alkyl, C₁-C₆ alkyl, or C₁-C₄ alkyl). In embodiments, R²² is an unsubstituted alkyl (e.g., C₁-C₈ alkyl, C₁-C₆ alkyl, or C₁-C₄ alkyl). In embodiments, R²² is a substituted or unsubstituted heteroalkyl (e.g., 2 to 8 membered heteroalkyl, 2 to 6 membered heteroalkyl, or 2 to 4 membered heteroalkyl). In embodiments, R²² is a substituted heteroalkyl (e.g., 2 to 8 membered heteroalkyl, 2 to 6 membered heteroalkyl, or 2 to 4 membered heteroalkyl). In embodiments, R²² is an unsubstituted heteroalkyl (e.g., 2 to 8 membered heteroalkyl, 2 to 6 membered heteroalkyl, or 2 to 4 membered heteroalkyl). In embodiments, R²² is a substituted or unsubstituted cycloalkyl (e.g., C₃-C₈ cycloalkyl, C₃-C₆ cycloalkyl, or C₅-C₆ cycloalkyl). In embodiments, R²² is a substituted cycloalkyl (e.g., C₃-C₈ cycloalkyl, C₃-C₆ cycloalkyl, or C₅-C₆ cycloalkyl). In embodiments, R²² is an unsubstituted cycloalkyl (e.g., C₃-C₈ cycloalkyl, C₃-C₆ cycloalkyl, or C₅-C₆ cycloalkyl). In embodiments, R²² is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R²² is a substituted heterocycloalkyl (e.g., 3 to 8 membered heterocycloalkyl, 3 to 6 membered heterocycloalkyl, or 5 to 6 membered heterocycloalkyl). In embodiments, R²² is an unsubstituted heterocycloalkyl (e.g., 3 to 8 membered heterocycloalkyl, 3 to 6 membered heterocycloalkyl, or 5 to 6 membered heterocycloalkyl). In embodiments, R²² is a substituted or unsubstituted aryl (e.g., C₆-C₁₀ aryl, C₁₀ aryl, or phenyl). In embodiments, R²² is a substituted aryl (e.g., C₆-C₁₀ aryl, C₁₀ aryl, or phenyl). In embodiments, R²² is an unsubstituted aryl (e.g., C₆-C₁₀ aryl, C₁₀ aryl, or phenyl). In embodiments, R²² is a substituted or unsubstituted heteroaryl (e.g., 5 to 10 membered heteroaryl, 5 to 9 membered heteroaryl, or 5 to 6 membered heteroaryl). In embodiments, R²² is a substituted heteroaryl (e.g., 5 to 10 membered heteroaryl, 5 to 9 membered heteroaryl, or 5 to 6 membered heteroaryl). In embodiments, R²² is an unsubstituted heteroaryl (e.g., 5 to 10 membered heteroaryl, 5 to 9 membered heteroaryl, or 5 to 6 membered heteroaryl).

In embodiments, when R²² is attached to nitrogen, R²² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₂H, —SOᵥ₂₂NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²² is hydrogen.

In embodiments, R²³ is independently halogen. In embodiments, R²³ is independently oxo. In embodiments, R²³ is independently —CF₃. In embodiments, R²³ is independently —CCl₃. In embodiments, R²³ is independently —CBr₃. In embodiments, R²³ is independently —Cl₃. In embodiments, R²³ is independently —CHF₂. In embodiments, R²³ is independently —CHCl₂. In embodiments, R²³ is independently —CHBr₂. In embodiments, R²³ is independently —CHI₂. In embodiments, R²³ is independently —CH₂F. In embodiments, R²³ is independently —CH₂Cl. In embodiments, R²³ is independently —CH₂Br. In embodiments, R²³ is independently —CH₂I. In embodiments, R²³ is independently —CN. In embodiments, R²³ is independently —N₃. In embodiments, R²³ is independently —SOₙ₂₃H. In embodiments, R²³ is independently —SOᵥ₂₃NH₂. In embodiments, R²³ is independently —NHNH₂. In embodiments, R²³ is independently —ONH₂. In embodiments, R²³ is independently —NHC(O)NHNH₂. In embodiments, R²³ is independently —NHC(O)NH₂. In embodiments, R²³ is independently —N(O)ₘ₂₃. In embodiments, R²³ is independently —NH₂. In embodiments, R²³ is independently —C(O)H. In embodiments, R²³ is independently —C(O)OH. In embodiments, R²³ is independently —C(O)NH₂. In embodiments, R²³ is independently —OH. In embodiments, R²³ is independently —NHSO₂H. In embodiments, R²³ is independently —NHC(O)H. In embodiments, R²³ is independently —NHC(O)OH. In embodiments, R²³ is independently —NHOH. In embodiments, R²³ is independently —OCF₃. In embodiments, R²³ is independently —OCCl₃. In embodiments, R²³ is independently —OCBr₃. In embodiments, R²³ is independently —OCl₃. In embodiments, R²³ is independently —OCHF₂. In embodiments, R²³ is independently —OCHCl₂. In embodiments, R²³ is independently —OCHBr₂. In embodiments, R²³ is independently —OCHI₂. In embodiments, R²³ is independently —OCH₂F. In embodiments, R²³ is independently —OCH₂Cl. In embodiments, R²³ is independently —OCH₂Br. In embodiments, R²³ is independently —OCH₂I.

In embodiments, R²³ is a substituted or unsubstituted alkyl (e.g., C₁-C₈ alkyl, C₁-C₆ alkyl, or C₁-C₄ alkyl). In embodiments, R²³ is a substituted alkyl (e.g., C₁-C₈ alkyl, C₁-C₆ alkyl, or C₁-C₄ alkyl). In embodiments, R²³ is an unsubstituted alkyl (e.g., C₁-C₈ alkyl, C₁-C₆ alkyl, or C₁-C₄ alkyl). In embodiments, R²³ is a substituted or unsubstituted heteroalkyl (e.g., 2 to 8 membered heteroalkyl, 2 to 6 membered heteroalkyl, or 2 to 4 membered heteroalkyl). In embodiments, R²³ is a substituted heteroalkyl (e.g., 2 to 8 membered heteroalkyl, 2 to 6 membered heteroalkyl, or 2 to 4 membered heteroalkyl). In embodiments, R²³ is an unsubstituted heteroalkyl (e.g., 2 to 8 membered heteroalkyl, 2 to 6 membered heteroalkyl, or 2 to 4 membered heteroalkyl). In embodiments, R²³ is a substituted or unsubstituted cycloalkyl (e.g., C₃-C₈ cycloalkyl, C₃-C₆ cycloalkyl, or C₅-C₆ cycloalkyl). In embodiments, R²³ is a substituted cycloalkyl (e.g., C₃-C₈ cycloalkyl, C₃-C₆ cycloalkyl, or C₅-C₆ cycloalkyl). In embodiments, R²³ is an unsubstituted cycloalkyl (e.g., C₃-C₈ cycloalkyl, C₃-C₆ cycloalkyl, or C₅-C₆ cycloalkyl). In embodiments, R²³ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R²³ is a substituted heterocycloalkyl (e.g., 3 to 8 membered heterocycloalkyl, 3 to 6 membered heterocycloalkyl, or 5 to 6 membered heterocycloalkyl). In embodiments, R²³ is an unsubstituted heterocycloalkyl (e.g., 3 to 8 membered heterocycloalkyl, 3 to 6 membered heterocycloalkyl, or 5 to 6 membered heterocycloalkyl). In embodiments, R²³ is a substituted or unsubstituted aryl (e.g., C₆-C₁₀ aryl, C₁₀ aryl, or phenyl). In embodiments, R²³ is a substituted aryl (e.g., C₆-C₁₀ aryl, C₁₀ aryl, or phenyl). In embodiments, R²³ is an unsubstituted aryl (e.g., C₆-C₁₀ aryl, C₁₀ aryl, or phenyl). In embodiments, R²³ is a substituted or unsubstituted heteroaryl (e.g., 5 to 10 membered heteroaryl, 5 to 9 membered heteroaryl, or 5 to 6 membered heteroaryl). In embodiments, R²³ is a substituted heteroaryl (e.g., 5 to 10 membered heteroaryl, 5 to 9 membered heteroaryl, or 5 to 6 membered heteroaryl). In embodiments, R²³ is an unsubstituted heteroaryl (e.g., 5 to 10 membered heteroaryl, 5 to 9 membered heteroaryl, or 5 to 6 membered heteroaryl).

In embodiments, R²⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₄H, —SOᵥ₂₄NH₂, —NHNH₂, —ONH₂, —NHC(O)NHNH₂, —NHC(O)NH₂, —N(O)ₘ₂₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²⁴ is halogen. In embodiments, R²⁴ is —F.

In embodiments, R²⁴ is a substituted or unsubstituted alkyl (e.g., C₁-C₈ alkyl, C₁-C₆ alkyl, or C₁-C₄ alkyl). In embodiments, R²⁴ is a substituted alkyl (e.g., C₁-C₈ alkyl, C₁-C₆ alkyl, or C₁-C₄ alkyl). In embodiments, R²⁴ is an unsubstituted alkyl (e.g., C₁-C₈ alkyl, C₁-C₆ alkyl, or C₁-C₄ alkyl). In embodiments, R²⁴ is a substituted or unsubstituted heteroalkyl (e.g., 2 to 8 membered heteroalkyl, 2 to 6 membered heteroalkyl, or 2 to 4 membered heteroalkyl). In embodiments, R²⁴ is a substituted heteroalkyl (e.g., 2 to 8 membered heteroalkyl, 2 to 6 membered heteroalkyl, or 2 to 4 membered heteroalkyl). In embodiments, R²⁴ is an unsubstituted heteroalkyl (e.g., 2 to 8 membered heteroalkyl, 2 to 6 membered heteroalkyl, or 2 to 4 membered heteroalkyl). In embodiments, R²⁴ is a substituted or unsubstituted cycloalkyl (e.g., C₃-C₈ cycloalkyl, C₃-C₆ cycloalkyl, or C₅-C₆ cycloalkyl). In embodiments, R²⁴ is a substituted cycloalkyl (e.g., C₃-C₈ cycloalkyl, C₃-C₆ cycloalkyl, or C₅-C₆ cycloalkyl). In embodiments, R²⁴ is an unsubstituted cycloalkyl (e.g., C₃-C₈ cycloalkyl, C₃-C₆ cycloalkyl, or C₅-C₆ cycloalkyl). In embodiments, R²⁴ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R²⁴ is a substituted heterocycloalkyl (e.g., 3 to 8 membered heterocycloalkyl, 3 to 6 membered heterocycloalkyl, or 5 to 6 membered heterocycloalkyl). In embodiments, R²⁴ is an unsubstituted heterocycloalkyl (e.g., 3 to 8 membered heterocycloalkyl, 3 to 6 membered heterocycloalkyl, or 5 to 6 membered heterocycloalkyl). In embodiments, R²⁴ is a substituted or unsubstituted aryl (e.g., C₆-C₁₀ aryl, C₁₀ aryl, or phenyl). In embodiments, R²⁴ is a substituted aryl (e.g., C₆-C₁₀ aryl, C₁₀ aryl, or phenyl). In embodiments, R²⁴ is an unsubstituted aryl (e.g., C₆-C₁₀ aryl, C₁₀ aryl, or phenyl). In embodiments, R²⁴ is a substituted or unsubstituted heteroaryl (e.g., 5 to 10 membered heteroaryl, 5 to 9 membered heteroaryl, or 5 to 6 membered heteroaryl). In embodiments, R²⁴ is a substituted heteroaryl (e.g., 5 to 10 membered heteroaryl, 5 to 9 membered heteroaryl, or 5 to 6 membered heteroaryl). In embodiments, R²⁴ is an unsubstituted heteroaryl (e.g., 5 to 10 membered heteroaryl, 5 to 9 membered heteroaryl, or 5 to 6 membered heteroaryl).

In embodiments, R²⁴ is hydrogen. In embodiments, R²⁴ is halogen. In embodiments, R²⁴ is —CF₃. In embodiments, R²⁴ is —CCl₃. In embodiments, R²⁴ is —CBr₃. In embodiments, R²⁴ is —Cl₃. In embodiments, R²⁴ is —CHF₂. In embodiments, R²⁴ is —CHCl₂. In embodiments, R²⁴ is —CHBr₂. In embodiments, R²⁴ is —CHI₂. In embodiments, R²⁴ is —CH₂F. In embodiments, R²⁴ is —CH₂Cl. In embodiments, R²⁴ is —CH₂Br. In embodiments, R²⁴ is —CH₂I. In embodiments, R²⁴ is —CN. In embodiments, R²⁴ is —SOₙ₂₄H. In embodiments, R²⁴ is —SOᵥ₂₄NH₂. In embodiments, R²⁴ is —NHNH₂. In embodiments, R²⁴ is —ONH₂. In embodiments, R²⁴ is —NHC(O)NHNH₂. In embodiments, R²⁴ is —NHC(O)NH₂. In embodiments, R²⁴ is —N(O)ₘ₂₄. In embodiments, R²⁴ is —NH₂. In embodiments, R²⁴ is —C(O)H. In embodiments, R²⁴ is —C(O)OH. In embodiments, R²⁴ is —C(O)NH₂. In embodiments, R²⁴ is —OH. In embodiments, R²⁴ is —NHSO₂H. In embodiments, R²⁴ is —NHC(O)H. In embodiments, R²⁴ is —NHC(O)OH. In embodiments, R²⁴ is —NHOH. In embodiments, R²⁴ is —OCF₃. In embodiments, R²⁴ is —OCCl₃. In embodiments, R²⁴ is —OCBr₃. In embodiments, R²⁴ is —OCl₃. In embodiments, R²⁴ is —OCHF₂. In embodiments, R²⁴ is —OCHCl₂. In embodiments, R²⁴ is —OCHBr₂. In embodiments, R²⁴ is —OCHI₂. In embodiments, R²⁴ is independently —OCH₂F. In embodiments, R²⁴ is independently —OCH₂Cl. In embodiments, R²⁴ is independently —OCH₂Br. In embodiments, R²⁴ is independently —OCH₂I.

In embodiments, R²⁴ and R²⁵ are independently —CN, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²⁴ and R²⁵ are independently —CH₂OH, —C(O)NH₂, —C(O)NHCH₃, —CH(OH)CH₂CH₃ or —CH(OH)CH₃. In embodiments, R²⁴ is —CF₃.

In embodiments, R²⁴ and R²⁵ are independently hydrogen or —CN.

In embodiments, R²⁴ and R²⁵ are independently hydrogen, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R²⁵ is halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₅R^{25A}, —SOᵥ₂₅NR^{25B}R^{25C}, —NHNR^{25B}R^{25C}, —ONR^{25B}R^{25C}, —NHC(O)NHNR^{25B}R^{25C}, —NHC(O)NR^{25B}R^{25C}, —N(O)ₘ₂₅, —NR^{25B}R^{25C}, —C(O)R^{25D}, —C(O)OR^{25D}, —C(O)NR^{25B}R^{25C}, —OR^{25A}, —NR^{25B}SO₂R^{25A}, —NR^{25B}C(O)R^{25D}, —NR^{25B}C(O)OR^{25D}, —NR^{25B}OR^{25D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). R^{25A}, R^{25B}, R^{25C}, and R^{25D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{25B} and R^{25C} substituents bonded to the same nitrogen atom may optionally be joined to form a substituted or unsubstituted heterocycloalkyl or substituted or unsubstituted heteroaryl. The symbol n25 is an integer from 0 to 4. The symbols m25 and v25 are independently 1 or 2. In embodiments, R²⁵ is —CF₃.

In embodiments, R²⁵ is hydrogen. In embodiments, R²⁵ is halogen. In embodiments, R²⁵ is —CF₃. In embodiments, R²⁵ is —CCl₃. In embodiments, R²⁵ is —CBr₃. In embodiments, R²⁵ is —Cl₃. In embodiments, R²⁵ is —CHF₂. In embodiments, R²⁵ is —CHCl₂. In embodiments, R²⁵ is —CHBr₂. In embodiments, R²⁵ is —CHI₂. In embodiments, R²⁵ is —CH₂F. In embodiments, R²⁵ is —CH₂Cl. In embodiments, R²⁵ is —CH₂Br. In embodiments, R²⁵ is —CH₂I. In embodiments, R²⁵ is —CN. In embodiments, R²⁵ is —SOₙ₂₅R^{25A}. In embodiments, R²⁵ is —SOᵥ₂₅NR^{25B}R^{25C}. In embodiments, R²⁵ is —NHNR^{25B}R^{25C}. In embodiments, R²⁵ is —ONR^{25B}R^{25C}. In embodiments, R²⁵ is —NHC(O)NHNR^{25B}R^{25C}. In embodiments, R²⁵ is —NHC(O)NR^{25B}R^{25C}. In embodiments, R²⁵ is —N(O)₂₅. In embodiments, R²⁵ is —NR^{25B}R^{25C}. In embodiments, R²⁵ is —C(O)R^{25D}. In embodiments, R²⁵ is —C(O)OR^{25D}. In embodiments, R²⁵ is —C(O)NR^{25B}R^{25C}. In embodiments, R²⁵ is —OR^{25A}. In embodiments, R²⁵ is —NR^{25B}SO₂R^{25A}. In embodiments, R²⁵ is —NR^{25B}C(O)R^{25D}. In embodiments, R²⁵ is —NR^{25B}C(O)OR^{25D}. In embodiments, R²⁵ is —NR^{25B}OR^{25D}. In embodiments, R²⁵ is —OCF₃. In embodiments, R²⁵ is —OCCl₃. In embodiments, R²⁵ is —OCBr₃. In embodiments, R²⁵ is —OCl₃. In embodiments, R²⁵ is —OCHF₂. In embodiments, R²⁵ is —OCHCl₂. In embodiments, R²⁵ is —OCHBr₂. In embodiments, R²⁵ is —OCHI₂. In embodiments, R²⁵ is independently —OCH₂F. In embodiments, R²⁵ is independently —OCH₂Cl. In embodiments, R²⁵ is independently —OCH₂Br. In embodiments, R²⁵ is independently —OCH₂I.

In embodiments, R²⁵ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₅R^{25A}, —SOᵥ₂₅NR^{25B}R^{25C}, —NHNR^{25B}R^{25C}, —ONR^{25B}R^{25C}, —NHC(O)NHNR^{25B}R^{25C}, —NHC(O)NR^{25B}R^{25C}, —N(O)ₘ₂₅, —NR^{25B}R^{25C}, —C(O)R^{25D}, —C(O)OR^{25D}, —C(O)NR^{25B}R^{25C}, —OR^{25A}, —NR^{25B}SO₂R^{25A}, —NR^{25B}C(O)R^{25D}, —NR^{25B}C(O)OR^{25D}, —NR^{25B}OR^{25D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R⁴⁷-substituted or unsubstituted alkyl (e.g., C₁-C₈ alkyl, C₁-C₆ alkyl, or C₁-C₄ alkyl), R⁴⁷-substituted or unsubstituted heteroalkyl (e.g., 2 to 8 membered heteroalkyl, 2 to 6 membered heteroalkyl, or 2 to 4 membered heteroalkyl), R⁴⁷-substituted or unsubstituted cycloalkyl (e.g., C₃-C₈ cycloalkyl, C₃-C₆ cycloalkyl, or C₅-C₆ cycloalkyl), R⁴⁷-substituted or unsubstituted heterocycloalkyl (e.g., 3 to 8 membered heterocycloalkyl, 3 to 6 membered heterocycloalkyl, or 5 to 6 membered heterocycloalkyl), R⁴⁷-substituted or unsubstituted aryl (e.g., C₆-C₁₀ aryl, C₁₀ aryl, or phenyl), or R⁴⁷-substituted or unsubstituted heteroaryl (e.g., 5 to 10 membered heteroaryl, 5 to 9 membered heteroaryl, or 5 to 6 membered heteroaryl).

In embodiments, R²⁵ is R⁴⁷-substituted or unsubstituted alkyl (e.g., C₁-C₈ alkyl, C₁-C₆ alkyl, or C₁-C₄ alkyl). In embodiments, R²⁵ is R⁴⁷-substituted alkyl (e.g., C₁-C₈ alkyl, C₁-C₆ alkyl, or C₁-C₄ alkyl). In embodiments, R²⁵ is an unsubstituted alkyl (e.g., C₁-C₈ alkyl, C₁-C₆ alkyl, or C₁-C₄ alkyl). In embodiments, R²⁵ is R⁴⁷-substituted or unsubstituted heteroalkyl (e.g., 2 to 8 membered heteroalkyl, 2 to 6 membered heteroalkyl, or 2 to 4 membered heteroalkyl). In embodiments, R²⁵ is R⁴⁷-substituted heteroalkyl (e.g., 2 to 8 membered heteroalkyl, 2 to 6 membered heteroalkyl, or 2 to 4 membered heteroalkyl). In embodiments, R²⁵ is an unsubstituted heteroalkyl (e.g., 2 to 8 membered heteroalkyl, 2 to 6 membered heteroalkyl, or 2 to 4 membered heteroalkyl). In embodiments, R²⁵ is R⁴⁷-substituted or unsubstituted cycloalkyl (e.g., C₃-C₈ cycloalkyl, C₃-C₆ cycloalkyl, or C₅-C₆ cycloalkyl). In embodiments, R²⁵ is R⁴⁷-substituted cycloalkyl (e.g., C₃-C₈ cycloalkyl, C₃-C₆ cycloalkyl, or C₅-C₆ cycloalkyl). In embodiments, R²⁵ is an unsubstituted cycloalkyl (e.g., C₃-C₈ cycloalkyl, C₃-C₆ cycloalkyl, or C₅-C₆ cycloalkyl). In embodiments, R²⁵ is R⁴⁷-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R²⁵ is R⁴⁷-substituted heterocycloalkyl (e.g., 3 to 8 membered heterocycloalkyl, 3 to 6 membered heterocycloalkyl, or 5 to 6 membered heterocycloalkyl). In embodiments, R²⁵ is an unsubstituted heterocycloalkyl (e.g., 3 to 8 membered heterocycloalkyl, 3 to 6 membered heterocycloalkyl, or 5 to 6 membered heterocycloalkyl). In embodiments, R²⁵ is R⁴⁷-substituted or unsubstituted aryl (e.g., C₆-C₁₀ aryl, C₁₀ aryl, or phenyl). In embodiments, R²⁵ is R⁴⁷-substituted aryl (e.g., C₆-C₁₀ aryl, C₁₀ aryl, or phenyl). In embodiments, R²⁵ is an unsubstituted aryl (e.g., C₆-C₁₀ aryl, C₁₀ aryl, or phenyl). In embodiments, R²⁵ is R⁴⁷-substituted or unsubstituted heteroaryl (e.g., 5 to 10 membered heteroaryl, 5 to 9 membered heteroaryl, or 5 to 6 membered heteroaryl). In embodiments, R²⁵ is R⁴⁷-substituted heteroaryl (e.g., 5 to 10 membered heteroaryl, 5 to 9 membered heteroaryl, or 5 to 6 membered heteroaryl). In embodiments, R²⁵ is an unsubstituted heteroaryl (e.g., 5 to 10 membered heteroaryl, 5 to 9 membered heteroaryl, or 5 to 6 membered heteroaryl).

R⁴⁷ is independently oxo, halogen, —CCl₃, —CBr₃, —CF₃, —CI₃, —CHF₂, —CHCl₂, —CHBr₂, —CHI₂, —CH₂F, —CH₂Cl, —CH₂Br, —CH₂I, CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂F, —OCH₂Cl, —OCH₂Br, —OCH₂I, R⁴⁸-substituted or unsubstituted alkyl (e.g., C₁-C₈ alkyl, C₁-C₆ alkyl, or C₁-C₄ alkyl), R⁴⁸-substituted or unsubstituted heteroalkyl (e.g., 2 to 8 membered heteroalkyl, 2 to 6 membered heteroalkyl, or 2 to 4 membered heteroalkyl), R⁴⁸-substituted or unsubstituted cycloalkyl (e.g., C₃-C₈ cycloalkyl, C₃-C₆ cycloalkyl, or C₅-C₆ cycloalkyl), R⁴⁸-substituted or unsubstituted heterocycloalkyl (e.g., 3 to 8 membered heterocycloalkyl, 3 to 6 membered heterocycloalkyl, or 5 to 6 membered heterocycloalkyl), R⁴⁸-substituted or unsubstituted aryl (e.g., C₆-C₁₀ aryl, C₁₀ aryl, or phenyl), or R⁴⁸-substituted or unsubstituted heteroaryl (e.g., 5 to 10 membered heteroaryl, 5 to 9 membered heteroaryl, or 5 to 6 membered heteroaryl).

In embodiments, R⁴⁷ is R⁴⁸-substituted or unsubstituted alkyl (e.g., C₁-C₈ alkyl, C₁-C₆ alkyl, or C₁-C₄ alkyl). In embodiments, R⁴⁷ is R⁴⁸-substituted alkyl (e.g., C₁-C₈ alkyl, C₁-C₆ alkyl, or C₁-C₄ alkyl). In embodiments, R⁴⁷ is an unsubstituted alkyl (e.g., C₁-C₈ alkyl, C₁-C₆ alkyl, or C₁-C₄ alkyl). In embodiments, R⁴⁷ is R⁴⁸-substituted or unsubstituted heteroalkyl (e.g., 2 to 8 membered heteroalkyl, 2 to 6 membered heteroalkyl, or 2 to 4 membered heteroalkyl). In embodiments, R⁴⁷ is R⁴⁸-substituted heteroalkyl (e.g., 2 to 8 membered heteroalkyl, 2 to 6 membered heteroalkyl, or 2 to 4 membered heteroalkyl). In embodiments, R⁴⁷ is an unsubstituted heteroalkyl (e.g., 2 to 8 membered heteroalkyl, 2 to 6 membered heteroalkyl, or 2 to 4 membered heteroalkyl). In embodiments, R⁴⁷ is R⁴⁸-substituted or unsubstituted cycloalkyl (e.g., C₃-C₈ cycloalkyl, C₃-C₆ cycloalkyl, or C₅-C₆ cycloalkyl). In embodiments, R⁴⁷ is R⁴⁸-substituted cycloalkyl (e.g., C₃-C₈ cycloalkyl, C₃-C₆ cycloalkyl, or C₅-C₆ cycloalkyl). In embodiments, R⁴⁷ is an unsubstituted cycloalkyl (e.g., C₃-C₈ cycloalkyl, C₃-C₆ cycloalkyl, or C₅-C₆ cycloalkyl). In embodiments, R⁴⁷ is R⁴⁸-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴⁷ is R⁴⁸-substituted heterocycloalkyl (e.g., 3 to 8 membered heterocycloalkyl, 3 to 6 membered heterocycloalkyl, or 5 to 6 membered heterocycloalkyl). In embodiments, R⁴⁷ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁴⁷ is R⁴⁸-substituted or unsubstituted aryl (e.g., C₆-C₁₀ aryl, C₁₀ aryl, or phenyl). In embodiments, R⁴⁷ is R⁴⁸-substituted aryl (e.g., C₆-C₁₀ aryl, C₁₀ aryl, or phenyl). In embodiments, R⁴⁷ is an unsubstituted aryl (e.g., C₆-C₁₀ aryl, C₁₀ aryl, or phenyl). In embodiments, R⁴⁷ is R⁴⁸-substituted or unsubstituted heteroaryl (e.g., 5 to 10 membered heteroaryl, 5 to 9 membered heteroaryl, or 5 to 6 membered heteroaryl). In embodiments, R⁴⁷ is R⁴⁸-substituted heteroaryl (e.g., 5 to 10 membered heteroaryl, 5 to 9 membered heteroaryl, or 5 to 6 membered heteroaryl). In embodiments, R⁴⁷ is an unsubstituted heteroaryl (e.g., 5 to 10 membered heteroaryl, 5 to 9 membered heteroaryl, or 5 to 6 membered heteroaryl).

R⁴⁸ is independently oxo, halogen, —CCl₃, —CBr₃, —CF₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂F, —OCH₂Cl, —OCH₂Br, —OCH₂I, R⁴⁹-substituted or unsubstituted alkyl (e.g., C₁-C₈ alkyl, C₁-C₆ alkyl, or C₁-C₄ alkyl), R⁴⁹-substituted or unsubstituted heteroalkyl (e.g., 2 to 8 membered heteroalkyl, 2 to 6 membered heteroalkyl, or 2 to 4 membered heteroalkyl), R⁴⁹-substituted or unsubstituted cycloalkyl (e.g., C₃-C₈ cycloalkyl, C₃-C₆ cycloalkyl, or C₅-C₆ cycloalkyl), R⁴⁹-substituted or unsubstituted heterocycloalkyl (e.g., 3 to 8 membered heterocycloalkyl, 3 to 6 membered heterocycloalkyl, or 5 to 6 membered heterocycloalkyl), R⁴⁹-substituted or unsubstituted aryl (e.g., C₆-C₁₀ aryl, C₁₀ aryl, or phenyl), or R⁴⁹-substituted or unsubstituted heteroaryl (e.g., 5 to 10 membered heteroaryl, 5 to 9 membered heteroaryl, or 5 to 6 membered heteroaryl).

In embodiments, R⁴⁸ is R⁴⁹-substituted or unsubstituted alkyl (e.g., C₁-C₈ alkyl, C₁-C₆ alkyl, or C₁-C₄ alkyl). In embodiments, R⁴⁸ is R⁴⁹-substituted alkyl (e.g., C₁-C₈ alkyl, C₁-C₆ alkyl, or C₁-C₄ alkyl). In embodiments, R⁴⁸ is an unsubstituted alkyl (e.g., C₁-C₈ alkyl, C₁-C₆ alkyl, or C₁-C₄ alkyl). In embodiments, R⁴⁸ is R⁴⁹-substituted or unsubstituted heteroalkyl (e.g., 2 to 8 membered heteroalkyl, 2 to 6 membered heteroalkyl, or 2 to 4 membered heteroalkyl). In embodiments, R⁴⁸ is R⁴⁹-substituted heteroalkyl (e.g., 2 to 8 membered heteroalkyl, 2 to 6 membered heteroalkyl, or 2 to 4 membered heteroalkyl). In embodiments, R⁴⁸ is an unsubstituted heteroalkyl (e.g., 2 to 8 membered heteroalkyl, 2 to 6 membered heteroalkyl, or 2 to 4 membered heteroalkyl). In embodiments, R⁴⁸ is R⁴⁹-substituted or unsubstituted cycloalkyl (e.g., C₃-C₈ cycloalkyl, C₃-C₆ cycloalkyl, or C₅-C₆ cycloalkyl). In embodiments, R⁴⁸ is R⁴⁹-substituted cycloalkyl (e.g., C₃-C₈ cycloalkyl, C₃-C₆ cycloalkyl, or C₅-C₆ cycloalkyl). In embodiments, R⁴⁸ is an unsubstituted cycloalkyl (e.g., C₃-C₈ cycloalkyl, C₃-C₆ cycloalkyl, or C₅-C₆ cycloalkyl). In embodiments, R⁴⁸ is R⁴⁹-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴⁸ is R⁴⁹-substituted heterocycloalkyl (e.g., 3 to 8 membered heterocycloalkyl, 3 to 6 membered heterocycloalkyl, or 5 to 6 membered heterocycloalkyl). In embodiments, R⁴⁸ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁴⁸ is R⁴⁹-substituted or unsubstituted aryl (e.g., C₆-C₁₀ aryl, C₁₀ aryl, or phenyl). In embodiments, R⁴⁸ is R⁴⁹-substituted aryl (e.g., C₆-C₁₀ aryl, C₁₀ aryl, or phenyl). In embodiments, R⁴⁸ is an unsubstituted aryl (e.g., C₆-C₁₀ aryl, C₁₀ aryl, or phenyl). In embodiments, R⁴⁸ is R⁴⁹-substituted or unsubstituted heteroaryl (e.g., 5 to 10 membered heteroaryl, 5 to 9 membered heteroaryl, or 5 to 6 membered heteroaryl). In embodiments, R⁴⁸ is R⁴⁹-substituted heteroaryl (e.g., 5 to 10 membered heteroaryl, 5 to 9 membered heteroaryl, or 5 to 6 membered heteroaryl). In embodiments, R⁴⁸ is an unsubstituted heteroaryl (e.g., 5 to 10 membered heteroaryl, 5 to 9 membered heteroaryl, or 5 to 6 membered heteroaryl).

In embodiments, R^{25A} is hydrogen, halogen, —CF₃, —CCl₃, —CBr₃, —CI₃, —COOH, —CONH₂, R⁴⁷-substituted or unsubstituted alkyl (e.g., C₁-C₈ alkyl, C₁-C₆ alkyl, or C₁-C₄ alkyl), R⁴⁷-substituted or unsubstituted heteroalkyl (e.g., 2 to 8 membered heteroalkyl, 2 to 6 membered heteroalkyl, or 2 to 4 membered heteroalkyl), R⁴⁷-substituted or unsubstituted cycloalkyl (e.g., C₃-C₈ cycloalkyl, C₃-C₆ cycloalkyl, or C₅-C₆ cycloalkyl), R⁴⁷-substituted or unsubstituted heterocycloalkyl (e.g., 3 to 8 membered heterocycloalkyl, 3 to 6 membered heterocycloalkyl, or 5 to 6 membered heterocycloalkyl), R⁴⁷-substituted or unsubstituted aryl (e.g., C₆-C₁₀ aryl, C₁₀ aryl, or phenyl), or R⁴⁷-substituted or unsubstituted heteroaryl (e.g., 5 to 10 membered heteroaryl, 5 to 9 membered heteroaryl, or 5 to 6 membered heteroaryl).

In embodiments, R^{25A} is R⁴⁷-substituted or unsubstituted alkyl (e.g., C₁-C₈ alkyl, C₁-C₆ alkyl, or C₁-C₄ alkyl). In embodiments, R^{25A} is R⁴⁷-substituted alkyl (e.g., C₁-C₈ alkyl, C₁-C₆ alkyl, or C₁-C₄ alkyl). In embodiments, R^{25A} is an unsubstituted alkyl (e.g., C₁-C₈ alkyl, C₁-C₆ alkyl, or C₁-C₄ alkyl). In embodiments, R^{25A} is R⁴⁷-substituted or unsubstituted heteroalkyl (e.g., 2 to 8 membered heteroalkyl, 2 to 6 membered heteroalkyl, or 2 to 4 membered heteroalkyl). In embodiments, R^{25A} is R⁴⁷-substituted heteroalkyl (e.g., 2 to 8 membered heteroalkyl, 2 to 6 membered heteroalkyl, or 2 to 4 membered heteroalkyl). In embodiments, R^{25A} is an unsubstituted heteroalkyl (e.g., 2 to 8 membered heteroalkyl, 2 to 6 membered heteroalkyl, or 2 to 4 membered heteroalkyl). In embodiments, R^{25A} is R⁴⁷-substituted or unsubstituted cycloalkyl (e.g., C₃-C₈ cycloalkyl, C₃-C₆ cycloalkyl, or C₅-C₆ cycloalkyl). In embodiments, R^{25A} is R⁴⁷-substituted cycloalkyl (e.g., C₃-C₈ cycloalkyl, C₃-C₆ cycloalkyl, or C₅-C₆ cycloalkyl). In embodiments, R^{25A} is an unsubstituted cycloalkyl (e.g., C₃-C₈ cycloalkyl, C₃-C₆ cycloalkyl, or C₅-C₆ cycloalkyl). In embodiments, R^{25A} is R⁴⁷-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{25A} is R⁴⁷-substituted heterocycloalkyl (e.g., 3 to 8 membered heterocycloalkyl, 3 to 6 membered heterocycloalkyl, or 5 to 6 membered heterocycloalkyl). In embodiments, R^{25A} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{25A} is R⁴⁷-substituted or unsubstituted aryl (e.g., C₆-C₁₀ aryl, C₁₀ aryl, or phenyl). In embodiments, R^{25A} is R⁴⁷-substituted aryl (e.g., C₆-C₁₀ aryl, C₁₀ aryl, or phenyl). In embodiments, R^{25A} is an unsubstituted aryl (e.g., C₆-C₁₀ aryl, C₁₀ aryl, or phenyl). In embodiments, R^{25A} is R⁴⁷-substituted or unsubstituted heteroaryl (e.g., 5 to 10 membered heteroaryl, 5 to 9 membered heteroaryl, or 5 to 6 membered heteroaryl). In embodiments, R^{25A} is R⁴⁷-substituted heteroaryl (e.g., 5 to 10 membered heteroaryl, 5 to 9 membered heteroaryl, or 5 to 6 membered heteroaryl). In embodiments, R^{25A} is an unsubstituted heteroaryl (e.g., 5 to 10 membered heteroaryl, 5 to 9 membered heteroaryl, or 5 to 6 membered heteroaryl).

In embodiments, R^{25B} is hydrogen, halogen, —CF₃, —CCl₃, —CBr₃, —CI₃, —COOH, —CONH₂, R⁴⁷-substituted or unsubstituted alkyl (e.g., C₁-C₈ alkyl, C₁-C₆ alkyl, or C₁-C₄ alkyl), R⁴⁷-substituted or unsubstituted heteroalkyl (e.g., 2 to 8 membered heteroalkyl, 2 to 6 membered heteroalkyl, or 2 to 4 membered heteroalkyl), R⁴⁷-substituted or unsubstituted cycloalkyl (e.g., C₃-C₈ cycloalkyl, C₃-C₆ cycloalkyl, or C₅-C₆ cycloalkyl), R⁴⁷-substituted or unsubstituted heterocycloalkyl (e.g., 3 to 8 membered heterocycloalkyl, 3 to 6 membered heterocycloalkyl, or 5 to 6 membered heterocycloalkyl), R⁴⁷-substituted or unsubstituted aryl (e.g., C₆-C₁₀ aryl, C₁₀ aryl, or phenyl), or R⁴⁷-substituted or unsubstituted heteroaryl (e.g., 5 to 10 membered heteroaryl, 5 to 9 membered heteroaryl, or 5 to 6 membered heteroaryl). In embodiments, R^{25B} and R^{25C} substituents bonded to the same nitrogen atom may optionally be joined to form a R⁴⁷⁻substituted or unsubstituted heterocycloalkyl (e.g., 3 to 8 membered heterocycloalkyl, 3 to 6 membered heterocycloalkyl, or 5 to 6 membered heterocycloalkyl) or R⁴⁷-substituted or unsubstituted heteroaryl (e.g., 5 to 10 membered heteroaryl, 5 to 9 membered heteroaryl, or 5 to 6 membered heteroaryl).

In embodiments, R^{25B} is R⁴⁷-substituted or unsubstituted alkyl (e.g., C₁-C₈ alkyl, C₁-C₆ alkyl, or C₁-C₄ alkyl). In embodiments, R^{25B} is R⁴⁷-substituted alkyl (e.g., C₁-C₈ alkyl, C₁-C₆ alkyl, or C₁-C₄ alkyl). In embodiments, R^{25B} is an unsubstituted alkyl (e.g., C₁-C₈ alkyl, C₁-C₆ alkyl, or C₁-C₄ alkyl). In embodiments, R^{25B} is R⁴⁷-substituted or unsubstituted heteroalkyl (e.g., 2 to 8 membered heteroalkyl, 2 to 6 membered heteroalkyl, or 2 to 4 membered heteroalkyl). In embodiments, R^{25B} is R⁴⁷-substituted heteroalkyl (e.g., 2 to 8 membered heteroalkyl, 2 to 6 membered heteroalkyl, or 2 to 4 membered heteroalkyl). In embodiments, R^{25B} is an unsubstituted heteroalkyl (e.g., 2 to 8 membered heteroalkyl, 2 to 6 membered heteroalkyl, or 2 to 4 membered heteroalkyl). In embodiments, R^{25B} is R⁴⁷-substituted or unsubstituted cycloalkyl (e.g., C₃-C₈ cycloalkyl, C₃-C₆ cycloalkyl, or C₅-C₆ cycloalkyl). In embodiments, R^{25B} is R⁴⁷-substituted cycloalkyl (e.g., C₃-C₈ cycloalkyl, C₃-C₆ cycloalkyl, or C₅-C₆ cycloalkyl). In embodiments, R^{25B} is an unsubstituted cycloalkyl (e.g., C₃-C₈ cycloalkyl, C₃-C₆ cycloalkyl, or C₅-C₆ cycloalkyl). In embodiments, R^{25B} is R⁴⁷-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{25B} is R⁴⁷-substituted heterocycloalkyl (e.g., 3 to 8 membered heterocycloalkyl, 3 to 6 membered heterocycloalkyl, or 5 to 6 membered heterocycloalkyl). In embodiments, R^{25B} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{25B} is R⁴⁷-substituted or unsubstituted aryl (e.g., C₆-C₁₀ aryl, C₁₀ aryl, or phenyl). In embodiments, R^{25B} is R⁴⁷-substituted aryl (e.g., C₆-C₁₀ aryl, C₁₀ aryl, or phenyl). In embodiments, R^{25B} is an unsubstituted aryl (e.g., C₆-C₁₀ aryl, C₁₀ aryl, or phenyl). In embodiments, R^{25B} is R⁴⁷-substituted or unsubstituted heteroaryl (e.g., 5 to 10 membered heteroaryl, 5 to 9 membered heteroaryl, or 5 to 6 membered heteroaryl). In embodiments, R^{25B} is R⁴⁷-substituted heteroaryl (e.g., 5 to 10 membered heteroaryl, 5 to 9 membered heteroaryl, or 5 to 6 membered heteroaryl). In embodiments, R^{25B} is an unsubstituted heteroaryl (e.g., 5 to 10 membered heteroaryl, 5 to 9 membered heteroaryl, or 5 to 6 membered heteroaryl).

In embodiments, R^{25C} is hydrogen, halogen, —CF₃, —CCl₃, —CBr₃, —CI₃, —COOH, —CONH₂, R⁴⁷-substituted or unsubstituted alkyl (e.g., C₁-C₈ alkyl, C₁-C₆ alkyl, or C₁-C₄ alkyl), R⁴⁷-substituted or unsubstituted heteroalkyl (e.g., 2 to 8 membered heteroalkyl, 2 to 6 membered heteroalkyl, or 2 to 4 membered heteroalkyl), R⁴⁷-substituted or unsubstituted cycloalkyl (e.g., C₃-C₈ cycloalkyl, C₃-C₆ cycloalkyl, or C₅-C₆ cycloalkyl), R⁴⁷-substituted or unsubstituted heterocycloalkyl (e.g., 3 to 8 membered heterocycloalkyl, 3 to 6 membered heterocycloalkyl, or 5 to 6 membered heterocycloalkyl), R⁴⁷-substituted or unsubstituted aryl (e.g., C₆-C₁₀ aryl, C₁₀ aryl, or phenyl), or R⁴⁷-substituted or unsubstituted heteroaryl (e.g., 5 to 10 membered heteroaryl, 5 to 9 membered heteroaryl, or 5 to 6 membered heteroaryl). In embodiments, R^{25B} and R^{25C} substituents bonded to the same nitrogen atom may optionally be joined to form a R⁴⁷⁻substituted or unsubstituted heterocycloalkyl (e.g., 3 to 8 membered heterocycloalkyl, 3 to 6 membered heterocycloalkyl, or 5 to 6 membered heterocycloalkyl) or R⁴⁷-substituted or unsubstituted heteroaryl (e.g., 5 to 10 membered heteroaryl, 5 to 9 membered heteroaryl, or 5 to 6 membered heteroaryl).

In embodiments, R^{25C} is R⁴⁷-substituted or unsubstituted alkyl (e.g., C₁-C₈ alkyl, C₁-C₆ alkyl, or C₁-C₄ alkyl). In embodiments, R^{25C} is R⁴⁷-substituted alkyl (e.g., C₁-C₈ alkyl, C₁-C₆ alkyl, or C₁-C₄ alkyl). In embodiments, R^{25C} is an unsubstituted alkyl (e.g., C₁-C₈ alkyl, C₁-C₆ alkyl, or C₁-C₄ alkyl). In embodiments, R^{25C} is R⁴⁷-substituted or unsubstituted heteroalkyl (e.g., 2 to 8 membered heteroalkyl, 2 to 6 membered heteroalkyl, or 2 to 4 membered heteroalkyl). In embodiments, R^{25C} is R⁴⁷-substituted heteroalkyl (e.g., 2 to 8 membered heteroalkyl, 2 to 6 membered heteroalkyl, or 2 to 4 membered heteroalkyl). In embodiments, R^{25C} is an unsubstituted heteroalkyl (e.g., 2 to 8 membered heteroalkyl, 2 to 6 membered heteroalkyl, or 2 to 4 membered heteroalkyl). In embodiments, R^{25C} is R⁴⁷-substituted or unsubstituted cycloalkyl (e.g., C₃-C₈ cycloalkyl, C₃-C₆ cycloalkyl, or C₅-C₆ cycloalkyl). In embodiments, R^{25C} is R⁴⁷-substituted cycloalkyl (e.g., C₃-C₈ cycloalkyl, C₃-C₆ cycloalkyl, or C₅-C₆ cycloalkyl). In embodiments, R^{25C} is an unsubstituted cycloalkyl (e.g., C₃-C₈ cycloalkyl, C₃-C₆ cycloalkyl, or C₅-C₆ cycloalkyl). In embodiments, R^{25C} is R⁴⁷-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{25C} is R⁴⁷-substituted heterocycloalkyl (e.g., 3 to 8 membered heterocycloalkyl, 3 to 6 membered heterocycloalkyl, or 5 to 6 membered heterocycloalkyl). In embodiments, R^{25C} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{25C} is R⁴⁷-substituted or unsubstituted aryl (e.g., C₆-C₁₀ aryl, C₁₀ aryl, or phenyl). In embodiments, R^{25C} is R⁴⁷-substituted aryl (e.g., C₆-C₁₀ aryl, C₁₀ aryl, or phenyl). In embodiments, R^{25C} is an unsubstituted aryl (e.g., C₆-C₁₀ aryl, C₁₀ aryl, or phenyl). In embodiments, R^{25C} is R⁴⁷-substituted or unsubstituted heteroaryl (e.g., 5 to 10 membered heteroaryl, 5 to 9 membered heteroaryl, or 5 to 6 membered heteroaryl). In embodiments, R^{25C} is R⁴⁷-substituted heteroaryl (e.g., 5 to 10 membered heteroaryl, 5 to 9 membered heteroaryl, or 5 to 6 membered heteroaryl). In embodiments, R^{25C} is an unsubstituted heteroaryl (e.g., 5 to 10 membered heteroaryl, 5 to 9 membered heteroaryl, or 5 to 6 membered heteroaryl).

In embodiments, R^{25D} is hydrogen, halogen, —CF₃, —CCl₃, —CBr₃, —CI₃, —COOH, —CONH₂, R⁴⁷-substituted or unsubstituted alkyl (e.g., C₁-C₈ alkyl, C₁-C₆ alkyl, or C₁-C₄ alkyl), R⁴⁷-substituted or unsubstituted heteroalkyl (e.g., 2 to 8 membered heteroalkyl, 2 to 6 membered heteroalkyl, or 2 to 4 membered heteroalkyl), R⁴⁷-substituted or unsubstituted cycloalkyl (e.g., C₃-C₈ cycloalkyl, C₃-C₆ cycloalkyl, or C₅-C₆ cycloalkyl), R⁴⁷-substituted or unsubstituted heterocycloalkyl (e.g., 3 to 8 membered heterocycloalkyl, 3 to 6 membered heterocycloalkyl, or 5 to 6 membered heterocycloalkyl), R⁴⁷-substituted or unsubstituted aryl (e.g., C₆-C₁₀ aryl, C₁₀ aryl, or phenyl), or R⁴⁷-substituted or unsubstituted heteroaryl (e.g., 5 to 10 membered heteroaryl, 5 to 9 membered heteroaryl, or 5 to 6 membered heteroaryl).

In embodiments, R^{25D} is R⁴⁷-substituted or unsubstituted alkyl (e.g., C₁-C₈ alkyl, C₁-C₆ alkyl, or C₁-C₄ alkyl). In embodiments, R^{25D} is R⁴⁷-substituted alkyl (e.g., C₁-C₈ alkyl, C₁-C₆ alkyl, or C₁-C₄ alkyl). In embodiments, R^{25D} is an unsubstituted alkyl (e.g., C₁-C₈ alkyl, C₁-C₆ alkyl, or C₁-C₄ alkyl). In embodiments, R^{25D} is R⁴⁷-substituted or unsubstituted heteroalkyl (e.g., 2 to 8 membered heteroalkyl, 2 to 6 membered heteroalkyl, or 2 to 4 membered heteroalkyl). In embodiments, R^{25D} is R⁴⁷-substituted heteroalkyl (e.g., 2 to 8 membered heteroalkyl, 2 to 6 membered heteroalkyl, or 2 to 4 membered heteroalkyl). In embodiments, R^{25D} is an unsubstituted heteroalkyl (e.g., 2 to 8 membered heteroalkyl, 2 to 6 membered heteroalkyl, or 2 to 4 membered heteroalkyl). In embodiments, R^{25D} is R⁴⁷-substituted or unsubstituted cycloalkyl (e.g., C₃-C₈ cycloalkyl, C₃-C₆ cycloalkyl, or C₅-C₆ cycloalkyl). In embodiments, R^{25D} is R⁴⁷-substituted cycloalkyl (e.g., C₃-C₈ cycloalkyl, C₃-C₆ cycloalkyl, or C₅-C₆ cycloalkyl). In embodiments, R^{25D} is an unsubstituted cycloalkyl (e.g., C₃-C₈ cycloalkyl, C₃-C₆ cycloalkyl, or C₅-C₆ cycloalkyl). In embodiments, R^{25D} is R⁴⁷-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{25D} is R⁴⁷-substituted heterocycloalkyl (e.g., 3 to 8 membered heterocycloalkyl, 3 to 6 membered heterocycloalkyl, or 5 to 6 membered heterocycloalkyl). In embodiments, R^{25D} is an unsubstituted heterocycloalkyl (e.g., 3 to 8 membered heterocycloalkyl, 3 to 6 membered heterocycloalkyl, or 5 to 6 membered heterocycloalkyl). In embodiments, R^{25D} is R⁴⁷-substituted or unsubstituted aryl (e.g., C₆-C₁₀ aryl, C₁₀ aryl, or phenyl). In embodiments, R^{25D} is R⁴⁷-substituted aryl (e.g., C₆-C₁₀ aryl, C₁₀ aryl, or phenyl). In embodiments, R^{25D} is an unsubstituted aryl (e.g., C₆-C₁₀ aryl, C₁₀ aryl, or phenyl). In embodiments, R^{25D} is R⁴⁷-substituted or unsubstituted heteroaryl (e.g., 5 to 10 membered heteroaryl, 5 to 9 membered heteroaryl, or 5 to 6 membered heteroaryl). In embodiments, R^{25D} is R⁴⁷-substituted heteroaryl (e.g., 5 to 10 membered heteroaryl, 5 to 9 membered heteroaryl, or 5 to 6 membered heteroaryl). In embodiments, R^{25D} is an unsubstituted heteroaryl (e.g., 5 to 10 membered heteroaryl, 5 to 9 membered heteroaryl, or 5 to 6 membered heteroaryl).

In embodiments, R²⁶ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₆R^{26A}, —SOᵥ₂₆NR^{26B}R^{26C}, —NHNR^{26B}R^{26C}, —ONR^{26B}R^{26C}, —OR^{26B}R^{26C}, —NHC(O)NHNR^{26B}R^{26C}, —NHC(O)NR^{26B}R^{26C}, —N(O)ₘ₂₆, —NR^{26B}R^{26C}, —C(O)R^{26D}, —C(O)OR^{26D}, —C(O)N^{26B}R^{26C}, —OR^{26A}, —NR^{26B}SO₂R^{26A}, —NR^{26B}C(O)R^{26D}, —NR^{26B}C(O)OR^{26D}, —NR^{26B}OR^{26D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R⁵⁰-substituted or unsubstituted alkyl (e.g., C₁-C₈ alkyl, C₁-C₆ alkyl, or C₁-C₄ alkyl), R⁵⁰-substituted or unsubstituted heteroalkyl (e.g., 2 to 8 membered heteroalkyl, 2 to 6 membered heteroalkyl, or 2 to 4 membered heteroalkyl), R⁵⁰-substituted or unsubstituted cycloalkyl (e.g., C₃-C₈ cycloalkyl, C₃-C₆ cycloalkyl, or C₅-C₆ cycloalkyl), R⁵⁰-substituted or unsubstituted heterocycloalkyl (e.g., 3 to 8 membered heterocycloalkyl, 3 to 6 membered heterocycloalkyl, or 5 to 6 membered heterocycloalkyl), R⁵⁰-substituted or unsubstituted aryl (e.g., C₆-C₁₀ aryl, C₁₀ aryl, or phenyl), or R⁵⁰-substituted or unsubstituted heteroaryl (e.g., 5 to 10 membered heteroaryl, 5 to 9 membered heteroaryl, or 5 to 6 membered heteroaryl).

In embodiments, R²⁶ is R⁵⁰-substituted or unsubstituted alkyl (e.g., C₁-C₈ alkyl, C₁-C₆ alkyl, or C₁-C₄ alkyl). In embodiments, R²⁶ is R⁵⁰-substituted alkyl (e.g., C₁-C₈ alkyl, C₁-C₆ alkyl, or C₁-C₄ alkyl). In embodiments, R²⁶ is an unsubstituted alkyl (e.g., C₁-C₈ alkyl, C₁-C₆ alkyl, or C₁-C₄ alkyl). In embodiments, R²⁶ is R⁵⁰-substituted or unsubstituted heteroalkyl (e.g., 2 to 8 membered heteroalkyl, 2 to 6 membered heteroalkyl, or 2 to 4 membered heteroalkyl). In embodiments, R²⁶ is R⁵⁰-substituted heteroalkyl (e.g., 2 to 8 membered heteroalkyl, 2 to 6 membered heteroalkyl, or 2 to 4 membered heteroalkyl). In embodiments, R²⁶ is an unsubstituted heteroalkyl (e.g., 2 to 8 membered heteroalkyl, 2 to 6 membered heteroalkyl, or 2 to 4 membered heteroalkyl). In embodiments, R²⁶ is R⁵⁰-substituted or unsubstituted cycloalkyl (e.g., C₃-C₈ cycloalkyl, C₃-C₆ cycloalkyl, or C₅-C₆ cycloalkyl). In embodiments, R²⁶ is R⁵⁰-substituted cycloalkyl (e.g., C₃-C₈ cycloalkyl, C₃-C₆ cycloalkyl, or C₅-C₆ cycloalkyl). In embodiments, R²⁶ is an unsubstituted cycloalkyl (e.g., C₃-C₈ cycloalkyl, C₃-C₆ cycloalkyl, or C₅-C₆ cycloalkyl). In embodiments, R²⁶ is R⁵⁰-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R²⁶ is R⁵⁰-substituted heterocycloalkyl (e.g., 3 to 8 membered heterocycloalkyl, 3 to 6 membered heterocycloalkyl, or 5 to 6 membered heterocycloalkyl). In embodiments, R²⁶ is an unsubstituted heterocycloalkyl (e.g., 3 to 8 membered heterocycloalkyl, 3 to 6 membered heterocycloalkyl, or 5 to 6 membered heterocycloalkyl). In embodiments, R²⁶ is R⁵⁰-substituted or unsubstituted aryl (e.g., C₆-C₁₀ aryl, C₁₀ aryl, or phenyl). In embodiments, R²⁶ is R⁵⁰-substituted aryl (e.g., C₆-C₁₀ aryl, C₁₀ aryl, or phenyl). In embodiments, R²⁶ is an unsubstituted aryl (e.g., C₆-C₁₀ aryl, C₁₀ aryl, or phenyl). In embodiments, R²⁶ is R⁵⁰-substituted or unsubstituted heteroaryl (e.g., 5 to 10 membered heteroaryl, 5 to 9 membered heteroaryl, or 5 to 6 membered heteroaryl). In embodiments, R²⁶ is R⁵⁰-substituted heteroaryl (e.g., 5 to 10 membered heteroaryl, 5 to 9 membered heteroaryl, or 5 to 6 membered heteroaryl). In embodiments, R²⁶ is an unsubstituted heteroaryl (e.g., 5 to 10 membered heteroaryl, 5 to 9 membered heteroaryl, or 5 to 6 membered heteroaryl).

R⁵⁰ is independently oxo, halogen, —CCl₃, —CBr₃, —CF₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂F, —OCH₂Cl, —OCH₂Br, —OCH₂I, R⁵¹-substituted or unsubstituted alkyl (e.g., C₁-C₈ alkyl, C₁-C₆ alkyl, or C₁-C₄ alkyl), R⁵¹-substituted or unsubstituted heteroalkyl (e.g., 2 to 8 membered heteroalkyl, 2 to 6 membered heteroalkyl, or 2 to 4 membered heteroalkyl), R⁵¹-substituted or unsubstituted cycloalkyl (e.g., C₃-C₈ cycloalkyl, C₃-C₆ cycloalkyl, or C₅-C₆ cycloalkyl), R⁵¹-substituted or unsubstituted heterocycloalkyl (e.g., 3 to 8 membered heterocycloalkyl, 3 to 6 membered heterocycloalkyl, or 5 to 6 membered heterocycloalkyl), R⁵¹-substituted or unsubstituted aryl (e.g., C₆-C₁₀ aryl, C₁₀ aryl, or phenyl), or R⁵¹-substituted or unsubstituted heteroaryl (e.g., 5 to 10 membered heteroaryl, 5 to 9 membered heteroaryl, or 5 to 6 membered heteroaryl).

In embodiments, R⁵⁰ is R⁵¹-substituted or unsubstituted alkyl (e.g., C₁-C₈ alkyl, C₁-C₆ alkyl, or C₁-C₄ alkyl). In embodiments, R⁵⁰ is R⁵¹-substituted alkyl (e.g., C₁-C₈ alkyl, C₁-C₆ alkyl, or C₁-C₄ alkyl). In embodiments, R⁵⁰ is an unsubstituted alkyl (e.g., C₁-C₈ alkyl, C₁-C₆ alkyl, or C₁-C₄ alkyl). In embodiments, R⁵⁰ is R⁵¹-substituted or unsubstituted heteroalkyl (e.g., 2 to 8 membered heteroalkyl, 2 to 6 membered heteroalkyl, or 2 to 4 membered heteroalkyl). In embodiments, R⁵⁰ is R⁵¹-substituted heteroalkyl (e.g., 2 to 8 membered heteroalkyl, 2 to 6 membered heteroalkyl, or 2 to 4 membered heteroalkyl). In embodiments, R⁵⁰ is an unsubstituted heteroalkyl (e.g., 2 to 8 membered heteroalkyl, 2 to 6 membered heteroalkyl, or 2 to 4 membered heteroalkyl). In embodiments, R⁵⁰ is R⁵¹-substituted or unsubstituted cycloalkyl (e.g., C₃-C₈ cycloalkyl, C₃-C₆ cycloalkyl, or C₅-C₆ cycloalkyl). In embodiments, R⁵⁰ is R⁵¹-substituted cycloalkyl (e.g., C₃-C₈ cycloalkyl, C₃-C₆ cycloalkyl, or C₅-C₆ cycloalkyl). In embodiments, R⁵⁰ is an unsubstituted cycloalkyl (e.g., C₃-C₈ cycloalkyl, C₃-C₆ cycloalkyl, or C₅-C₆ cycloalkyl). In embodiments, R⁵⁰ is R⁵¹-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁵⁰ is R⁵¹-substituted heterocycloalkyl (e.g., 3 to 8 membered heterocycloalkyl, 3 to 6 membered heterocycloalkyl, or 5 to 6 membered heterocycloalkyl). In embodiments, R⁵⁰ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁵⁰ is R⁵¹-substituted or unsubstituted aryl (e.g., C₆-C₁₀ aryl, C₁₀ aryl, or phenyl). In embodiments, R⁵⁰ is R⁵¹-substituted aryl (e.g., C₆-C₁₀ aryl, C₁₀ aryl, or phenyl). In embodiments, R⁵⁰ is an unsubstituted aryl (e.g., C₆-C₁₀ aryl, C₁₀ aryl, or phenyl). In embodiments, R⁵⁰ is R⁵¹-substituted or unsubstituted heteroaryl (e.g., 5 to 10 membered heteroaryl, 5 to 9 membered heteroaryl, or 5 to 6 membered heteroaryl). In embodiments, R⁵⁰ is R⁵¹-substituted heteroaryl (e.g., 5 to 10 membered heteroaryl, 5 to 9 membered heteroaryl, or 5 to 6 membered heteroaryl). In embodiments, R⁵⁰ is an unsubstituted heteroaryl (e.g., 5 to 10 membered heteroaryl, 5 to 9 membered heteroaryl, or 5 to 6 membered heteroaryl).

R⁵¹ is independently oxo, halogen, —CCl₃, —CBr₃, —CF₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂F, —OCH₂Cl, —OCH₂Br, —OCH₂I, R⁵²-substituted or unsubstituted alkyl (e.g., C₁-C₈ alkyl, C₁-C₆ alkyl, or C₁-C₄ alkyl), R⁵²-substituted or unsubstituted heteroalkyl (e.g., 2 to 8 membered heteroalkyl, 2 to 6 membered heteroalkyl, or 2 to 4 membered heteroalkyl), R⁵²-substituted or unsubstituted cycloalkyl (e.g., C₃-C₈ cycloalkyl, C₃-C₆ cycloalkyl, or C₅-C₆ cycloalkyl), R⁵²-substituted or unsubstituted heterocycloalkyl (e.g., 3 to 8 membered heterocycloalkyl, 3 to 6 membered heterocycloalkyl, or 5 to 6 membered heterocycloalkyl), R⁵²-substituted or unsubstituted aryl (e.g., C₆-C₁₀ aryl, C₁₀ aryl, or phenyl), or R⁵²-substituted or unsubstituted heteroaryl (e.g., 5 to 10 membered heteroaryl, 5 to 9 membered heteroaryl, or 5 to 6 membered heteroaryl).

In embodiments, R⁵¹ is R⁵²-substituted or unsubstituted alkyl (e.g., C₁-C₈ alkyl, C₁-C₆ alkyl, or C₁-C₄ alkyl). In embodiments, R⁵¹ is R⁵²-substituted alkyl (e.g., C₁-C₈ alkyl, C₁-C₆ alkyl, or C₁-C₄ alkyl). In embodiments, R⁵¹ is an unsubstituted alkyl (e.g., C₁-C₈ alkyl, C₁-C₆ alkyl, or C₁-C₄ alkyl). In embodiments, R⁵¹ is R⁵²-substituted or unsubstituted heteroalkyl (e.g., 2 to 8 membered heteroalkyl, 2 to 6 membered heteroalkyl, or 2 to 4 membered heteroalkyl). In embodiments, R⁵¹ is R⁵²-substituted heteroalkyl (e.g., 2 to 8 membered heteroalkyl, 2 to 6 membered heteroalkyl, or 2 to 4 membered heteroalkyl). In embodiments, R⁵¹ is an unsubstituted heteroalkyl (e.g., 2 to 8 membered heteroalkyl, 2 to 6 membered heteroalkyl, or 2 to 4 membered heteroalkyl). In embodiments, R⁵¹ is R⁵²-substituted or unsubstituted cycloalkyl (e.g., C₃-C₈ cycloalkyl, C₃-C₆ cycloalkyl, or C₅-C₆ cycloalkyl). In embodiments, R⁵¹ is R⁵²-substituted cycloalkyl (e.g., C₃-C₈ cycloalkyl, C₃-C₆ cycloalkyl, or C₅-C₆ cycloalkyl). In embodiments, R⁵¹ is an unsubstituted cycloalkyl (e.g., C₃-C₈ cycloalkyl, C₃-C₆ cycloalkyl, or C₅-C₆ cycloalkyl). In embodiments, R⁵¹ is R⁵²-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁵¹ is R⁵²-substituted heterocycloalkyl (e.g., 3 to 8 membered heterocycloalkyl, 3 to 6 membered heterocycloalkyl, or 5 to 6 membered heterocycloalkyl). In embodiments, R⁵¹ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁵¹ is R⁵²-substituted or unsubstituted aryl (e.g., C₆-C₁₀ aryl, C₁₀ aryl, or phenyl). In embodiments, R⁵¹ is R⁵²-substituted aryl (e.g., C₆-C₁₀ aryl, C₁₀ aryl, or phenyl). In embodiments, R⁵¹ is an unsubstituted aryl (e.g. C₆-C₁₀ aryl, C₁₀ aryl, or phenyl). In embodiments, R⁵¹ is R⁵²-substituted or unsubstituted heteroaryl (e.g., 5 to 10 membered heteroaryl, 5 to 9 membered heteroaryl, or 5 to 6 membered heteroaryl). In embodiments, R⁵¹ is R⁵²-substituted heteroaryl (e.g., 5 to 10 membered heteroaryl, 5 to 9 membered heteroaryl, or 5 to 6 membered heteroaryl). In embodiments, R⁵¹ is an unsubstituted heteroaryl (e.g., 5 to 10 membered heteroaryl, 5 to 9 membered heteroaryl, or 5 to 6 membered heteroaryl).

In embodiments, R^{26A} is hydrogen, halogen, —CF₃, —CCl₃, —CBr₃, —CI₃, —COOH, —CONH₂, R⁵⁰-substituted or unsubstituted alkyl (e.g., C₁-C₈ alkyl, C₁-C₆ alkyl, or C₁-C₄ alkyl), R⁵⁰-substituted or unsubstituted heteroalkyl (e.g., 2 to 8 membered heteroalkyl, 2 to 6 membered heteroalkyl, or 2 to 4 membered heteroalkyl), R⁵⁰-substituted or unsubstituted cycloalkyl (e.g., C₃-C₈ cycloalkyl, C₃-C₆ cycloalkyl, or C₅-C₆ cycloalkyl), R⁵⁰-substituted or unsubstituted heterocycloalkyl (e.g., 3 to 8 membered heterocycloalkyl, 3 to 6 membered heterocycloalkyl, or 5 to 6 membered heterocycloalkyl), R⁵⁰-substituted or unsubstituted aryl (e.g., C₆-C₁₀ aryl, C₁₀ aryl, or phenyl), or R⁵⁰-substituted or unsubstituted heteroaryl (e.g., 5 to 10 membered heteroaryl, 5 to 9 membered heteroaryl, or 5 to 6 membered heteroaryl).

In embodiments, R^{26B} is hydrogen, halogen, —CF₃, —CCl₃, —CBr₃, —CI₃, —COOH, —CONH₂, R⁵⁰-substituted or unsubstituted alkyl (e.g., C₁-C₈ alkyl, C₁-C₆ alkyl, or C₁-C₄ alkyl), R⁵⁰-substituted or unsubstituted heteroalkyl (e.g., 2 to 8 membered heteroalkyl, 2 to 6 membered heteroalkyl, or 2 to 4 membered heteroalkyl), R⁵⁰-substituted or unsubstituted cycloalkyl (e.g., C₃-C₈ cycloalkyl, C₃-C₆ cycloalkyl, or C₅-C₆ cycloalkyl), R⁵⁰-substituted or unsubstituted heterocycloalkyl (e.g., 3 to 8 membered heterocycloalkyl, 3 to 6 membered heterocycloalkyl, or 5 to 6 membered heterocycloalkyl), R⁵⁰-substituted or unsubstituted aryl (e.g., C₆-C₁₀ aryl, C₁₀ aryl, or phenyl), or R⁵⁰-substituted or unsubstituted heteroaryl (e.g., 5 to 10 membered heteroaryl, 5 to 9 membered heteroaryl, or 5 to 6 membered heteroaryl). In embodiments, R^{26B} and R^{26C} substituents bonded to the same nitrogen atom may optionally be joined to form a R⁵⁰-substituted or unsubstituted heterocycloalkyl (e.g., 3 to 8 membered heterocycloalkyl, 3 to 6 membered heterocycloalkyl, or 5 to 6 membered heterocycloalkyl) or R⁵⁰-substituted or unsubstituted heteroaryl (e.g., 5 to 10 membered heteroaryl, 5 to 9 membered heteroaryl, or 5 to 6 membered heteroaryl).

In embodiments, R^{26C} is hydrogen, halogen, —CF₃, —CCl₃, —CBr₃, —CI₃, —COOH, —CONH₂, R⁵⁰-substituted or unsubstituted alkyl (e.g., C₁-C₈ alkyl, C₁-C₆ alkyl, or C₁-C₄ alkyl), R⁵⁰-substituted or unsubstituted heteroalkyl (e.g., 2 to 8 membered heteroalkyl, 2 to 6 membered heteroalkyl, or 2 to 4 membered heteroalkyl), R⁵⁰-substituted or unsubstituted cycloalkyl (e.g., C₃-C₈ cycloalkyl, C₃-C₆ cycloalkyl, or C₅-C₆ cycloalkyl), R⁵⁰-substituted or unsubstituted heterocycloalkyl (e.g., 3 to 8 membered heterocycloalkyl, 3 to 6 membered heterocycloalkyl, or 5 to 6 membered heterocycloalkyl), R⁵⁰-substituted or unsubstituted aryl (e.g., C₆-C₁₀ aryl, C₁₀ aryl, or phenyl), or R⁵⁰-substituted or unsubstituted heteroaryl (e.g., 5 to 10 membered heteroaryl, 5 to 9 membered heteroaryl, or 5 to 6 membered heteroaryl). In embodiments, R^{26B} and R^{26C} substituents bonded to the same nitrogen atom may optionally be joined to form a R⁵⁰-substituted or unsubstituted heterocycloalkyl (e.g., 3 to 8 membered heterocycloalkyl, 3 to 6 membered heterocycloalkyl, or 5 to 6 membered heterocycloalkyl) or R⁵⁰-substituted or unsubstituted heteroaryl (e.g., 5 to 10 membered heteroaryl, 5 to 9 membered heteroaryl, or 5 to 6 membered heteroaryl).

In embodiments, R^{26D} is hydrogen, halogen, —CF₃, —CCl₃, —CBr₃, —CI₃, —COOH, —CONH₂, R⁵⁰-substituted or unsubstituted alkyl (e.g., C₁-C₈ alkyl, C₁-C₆ alkyl, or C₁-C₄ alkyl), R⁵⁰-substituted or unsubstituted heteroalkyl (e.g., 2 to 8 membered heteroalkyl, 2 to 6 membered heteroalkyl, or 2 to 4 membered heteroalkyl), R⁵⁰-substituted or unsubstituted cycloalkyl (e.g., C₃-C₈ cycloalkyl, C₃-C₆ cycloalkyl, or C₅-C₆ cycloalkyl), R⁵⁰-substituted or unsubstituted heterocycloalkyl (e.g., 3 to 8 membered heterocycloalkyl, 3 to 6 membered heterocycloalkyl, or 5 to 6 membered heterocycloalkyl), R⁵⁰-substituted or unsubstituted aryl (e.g., C₆-C₁₀ aryl, C₁₀ aryl, or phenyl), or R⁵⁰-substituted or unsubstituted heteroaryl (e.g., 5 to 10 membered heteroaryl, 5 to 9 membered heteroaryl, or 5 to 6 membered heteroaryl).

In embodiments, R²⁶ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²⁶ is hydrogen, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R²⁷ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₇R^{27A}, —SOᵥ₂₇NR^{27B}R^{27C}, —NHNR^{27B}R^{27C}, —ONR^{27B}R^{27C}, —NHC(O)NHNR^{27B}R^{27C}, —NHC(O)NR^{27B}R^{27C}, —N(O)ₘ₂₇, —NR^{27B}R^{27C}, —C(O)R^{27D}, —C(O)OR^{27D}, —C(O)NR^{27B}R^{27C}, —OR^{27A}, —NR^{27B}SO₂R^{27A}, —NR^{27B}C(O)R^{27D}, —NR^{27B}C(O)OR^{27D}, —NR^{27B}OR^{27D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R⁵³-substituted or unsubstituted alkyl (e.g., C₁-C₈ alkyl, C₁-C₆ alkyl, or C₁-C₄ alkyl), R⁵³-substituted or unsubstituted heteroalkyl (e.g., 2 to 8 membered heteroalkyl, 2 to 6 membered heteroalkyl, or 2 to 4 membered heteroalkyl), R⁵³-substituted or unsubstituted cycloalkyl (e.g., C₃-C₈ cycloalkyl, C₃-C₆ cycloalkyl, or C₅-C₆ cycloalkyl), R⁵³-substituted or unsubstituted heterocycloalkyl (e.g., 3 to 8 membered heterocycloalkyl, 3 to 6 membered heterocycloalkyl, or 5 to 6 membered heterocycloalkyl), R⁵³-substituted or unsubstituted aryl (e.g., C₆-C₁₀ aryl, C₁₀ aryl, or phenyl), or R⁵³-substituted or unsubstituted heteroaryl (e.g., 5 to 10 membered heteroaryl, 5 to 9 membered heteroaryl, or 5 to 6 membered heteroaryl).

In embodiments, R²⁷ is R⁵³-substituted or unsubstituted alkyl (e.g., C₁-C₈ alkyl, C₁-C₆ alkyl, or C₁-C₄ alkyl). In embodiments, R²⁷ is R⁵³-substituted alkyl (e.g., C₁-C₈ alkyl, C₁-C₆ alkyl, or C₁-C₄ alkyl). In embodiments, R²⁷ is an unsubstituted alkyl (e.g., C₁-C₈ alkyl, C₁-C₆ alkyl, or C₁-C₄ alkyl). In embodiments, R²⁷ is R⁵³-substituted or unsubstituted heteroalkyl (e.g., 2 to 8 membered heteroalkyl, 2 to 6 membered heteroalkyl, or 2 to 4 membered heteroalkyl). In embodiments, R²⁷ is R⁵³-substituted heteroalkyl (e.g., 2 to 8 membered heteroalkyl, 2 to 6 membered heteroalkyl, or 2 to 4 membered heteroalkyl). In embodiments, R²⁷ is an unsubstituted heteroalkyl (e.g., 2 to 8 membered heteroalkyl, 2 to 6 membered heteroalkyl, or 2 to 4 membered heteroalkyl). In embodiments, R²⁷ is R⁵³-substituted or unsubstituted cycloalkyl (e.g., C₃-C₈ cycloalkyl, C₃-C₆ cycloalkyl, or C₅-C₆ cycloalkyl). In embodiments, R²⁷ is R⁵³-substituted cycloalkyl (e.g., C₃-C₈ cycloalkyl, C₃-C₆ cycloalkyl, or C₅-C₆ cycloalkyl). In embodiments, R²⁷ is an unsubstituted cycloalkyl (e.g., C₃-C₈ cycloalkyl, C₃-C₆ cycloalkyl, or C₅-C₆ cycloalkyl). In embodiments, R²⁷ is R⁵³-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R²⁷ is R⁵³-substituted heterocycloalkyl (e.g., 3 to 8 membered heterocycloalkyl, 3 to 6 membered heterocycloalkyl, or 5 to 6 membered heterocycloalkyl). In embodiments, R²⁷ is an unsubstituted heterocycloalkyl (e.g., 3 to 8 membered heterocycloalkyl, 3 to 6 membered heterocycloalkyl, or 5 to 6 membered heterocycloalkyl). In embodiments, R²⁷ is R⁵³-substituted or unsubstituted aryl (e.g., C₆-C₁₀ aryl, C₁₀ aryl, or phenyl). In embodiments, R²⁷ is R⁵³-substituted aryl (e.g., C₆-C₁₀ aryl, C₁₀ aryl, or phenyl). In embodiments, R²⁷ is an unsubstituted aryl (e.g., C₆-C₁₀ aryl, C₁₀ aryl, or phenyl). In embodiments, R²⁷ is R⁵³-substituted or unsubstituted heteroaryl (e.g., 5 to 10 membered heteroaryl, 5 to 9 membered heteroaryl, or 5 to 6 membered heteroaryl). In embodiments, R²⁷ is R⁵³-substituted heteroaryl (e.g., 5 to 10 membered heteroaryl, 5 to 9 membered heteroaryl, or 5 to 6 membered heteroaryl). In embodiments, R²⁷ is an unsubstituted heteroaryl (e.g., 5 to 10 membered heteroaryl, 5 to 9 membered heteroaryl, or 5 to 6 membered heteroaryl).

R⁵³ is independently oxo, halogen, —CCl₃, —CBr₃, —CF₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂F, —OCH₂Cl, —OCH₂Br, —OCH₂I, R⁵⁴-substituted or unsubstituted alkyl (e.g., C₁-C₈ alkyl, C₁-C₆ alkyl, or C₁-C₄ alkyl), R⁵⁴-substituted or unsubstituted heteroalkyl (e.g., 2 to 8 membered heteroalkyl, 2 to 6 membered heteroalkyl, or 2 to 4 membered heteroalkyl), R⁵⁴-substituted or unsubstituted cycloalkyl (e.g., C₃-C₈ cycloalkyl, C₃-C₆ cycloalkyl, or C₅-C₆ cycloalkyl), R⁵⁴-substituted or unsubstituted heterocycloalkyl (e.g., 3 to 8 membered heterocycloalkyl, 3 to 6 membered heterocycloalkyl, or 5 to 6 membered heterocycloalkyl), R⁵⁴-substituted or unsubstituted aryl (e.g., C₆-C₁₀ aryl, C₁₀ aryl, or phenyl), or R⁵⁴-substituted or unsubstituted heteroaryl (e.g., 5 to 10 membered heteroaryl, 5 to 9 membered heteroaryl, or 5 to 6 membered heteroaryl).

In embodiments, R⁵³ is R⁵⁴-substituted or unsubstituted alkyl (e.g., C₁-C₈ alkyl, C₁-C₆ alkyl, or C₁-C₄ alkyl). In embodiments, R⁵³ is R⁵⁴-substituted alkyl (e.g., C₁-C₈ alkyl, C₁-C₆ alkyl, or C₁-C₄ alkyl). In embodiments, R⁵³ is an unsubstituted alkyl (e.g., C₁-C₈ alkyl, C₁-C₆ alkyl, or C₁-C₄ alkyl). In embodiments, R⁵³ is R⁵⁴-substituted or unsubstituted heteroalkyl (e.g., 2 to 8 membered heteroalkyl, 2 to 6 membered heteroalkyl, or 2 to 4 membered heteroalkyl). In embodiments, R⁵³ is R⁵⁴-substituted heteroalkyl (e.g., 2 to 8 membered heteroalkyl, 2 to 6 membered heteroalkyl, or 2 to 4 membered heteroalkyl). In embodiments, R⁵³ is an unsubstituted heteroalkyl (e.g., 2 to 8 membered heteroalkyl, 2 to 6 membered heteroalkyl, or 2 to 4 membered heteroalkyl). In embodiments, R⁵³ is R⁵⁴-substituted or unsubstituted cycloalkyl (e.g., C₃-C₈ cycloalkyl, C₃-C₆ cycloalkyl, or C₅-C₆ cycloalkyl). In embodiments, R⁵³ is R⁵⁴-substituted cycloalkyl (e.g., C₃-C₈ cycloalkyl, C₃-C₆ cycloalkyl, or C₅-C₆ cycloalkyl). In embodiments, R⁵³ is an unsubstituted cycloalkyl (e.g., C₃-C₈ cycloalkyl, C₃-C₆ cycloalkyl, or C₅-C₆ cycloalkyl). In embodiments, R⁵³ is R⁵⁴-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁵³ is R⁵⁴-substituted heterocycloalkyl (e.g., 3 to 8 membered heterocycloalkyl, 3 to 6 membered heterocycloalkyl, or 5 to 6 membered heterocycloalkyl). In embodiments, R⁵³ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁵³ is R⁵⁴-substituted or unsubstituted aryl (e.g., C₆-C₁₀ aryl, C₁₀ aryl, or phenyl). In embodiments, R⁵³ is R⁵⁴-substituted aryl (e.g., C₆-C₁₀ aryl, C₁₀ aryl, or phenyl). In embodiments, R⁵³ is an unsubstituted aryl (e.g., C₆-C₁₀ aryl, C₁₀ aryl, or phenyl). In embodiments, R⁵³ is R⁵⁴-substituted or unsubstituted heteroaryl (e.g., 5 to 10 membered heteroaryl, 5 to 9 membered heteroaryl, or 5 to 6 membered heteroaryl). In embodiments, R⁵³ is R⁵⁴-substituted heteroaryl (e.g., 5 to 10 membered heteroaryl, 5 to 9 membered heteroaryl, or 5 to 6 membered heteroaryl). In embodiments, R⁵³ is an unsubstituted heteroaryl (e.g., 5 to 10 membered heteroaryl, 5 to 9 membered heteroaryl, or 5 to 6 membered heteroaryl).

R⁵⁴ is independently oxo, halogen, —CCl₃, —CBr₃, —CF₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂F, —OCH₂Cl, —OCH₂Br, —OCH₂I, R⁵⁵-substituted or unsubstituted alkyl (e.g., C₁-C₈ alkyl, C₁-C₆ alkyl, or C₁-C₄ alkyl), R⁵⁵-substituted or unsubstituted heteroalkyl (e.g., 2 to 8 membered heteroalkyl, 2 to 6 membered heteroalkyl, or 2 to 4 membered heteroalkyl), R⁵⁵-substituted or unsubstituted cycloalkyl (e.g., C₃-C₈ cycloalkyl, C₃-C₆ cycloalkyl, or C₅-C₆ cycloalkyl), R⁵⁵-substituted or unsubstituted heterocycloalkyl (e.g., 3 to 8 membered heterocycloalkyl, 3 to 6 membered heterocycloalkyl, or 5 to 6 membered heterocycloalkyl), R⁵⁵-substituted or unsubstituted aryl (e.g., C₆-C₁₀ aryl, C₁₀ aryl, or phenyl), or R⁵⁵-substituted or unsubstituted heteroaryl (e.g., 5 to 10 membered heteroaryl, 5 to 9 membered heteroaryl, or 5 to 6 membered heteroaryl).

In embodiments, R⁵⁴ is R⁵⁵-substituted or unsubstituted alkyl (e.g., C₁-C₈ alkyl, C₁-C₆ alkyl, or C₁-C₄ alkyl). In embodiments, R⁵⁴ is R⁵⁵-substituted alkyl (e.g., C₁-C₈ alkyl, C₁-C₆ alkyl, or C₁-C₄ alkyl). In embodiments, R⁵⁴ is an unsubstituted alkyl (e.g., C₁-C₈ alkyl, C₁-C₆ alkyl, or C₁-C₄ alkyl). In embodiments, R⁵⁴ is R⁵⁵-substituted or unsubstituted heteroalkyl (e.g., 2 to 8 membered heteroalkyl, 2 to 6 membered heteroalkyl, or 2 to 4 membered heteroalkyl). In embodiments, R⁵⁴ is R⁵⁵-substituted heteroalkyl (e.g., 2 to 8 membered heteroalkyl, 2 to 6 membered heteroalkyl, or 2 to 4 membered heteroalkyl). In embodiments, R⁵⁴ is an unsubstituted heteroalkyl (e.g., 2 to 8 membered heteroalkyl, 2 to 6 membered heteroalkyl, or 2 to 4 membered heteroalkyl). In embodiments, R⁵⁴ is R⁵⁵-substituted or unsubstituted cycloalkyl (e.g., C₃-C₈ cycloalkyl, C₃-C₆ cycloalkyl, or C₅-C₆ cycloalkyl). In embodiments, R⁵⁴ is R⁵⁵-substituted cycloalkyl (e.g., C₃-C₈ cycloalkyl, C₃-C₆ cycloalkyl, or C₅-C₆ cycloalkyl). In embodiments, R⁵⁴ is an unsubstituted cycloalkyl (e.g., C₃-C₈ cycloalkyl, C₃-C₆ cycloalkyl, or C₅-C₆ cycloalkyl). In embodiments, R⁵⁴ is R⁵⁵-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁵⁴ is R⁵⁵-substituted heterocycloalkyl (e.g., 3 to 8 membered heterocycloalkyl, 3 to 6 membered heterocycloalkyl, or 5 to 6 membered heterocycloalkyl). In embodiments, R⁵⁴ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁵⁴ is R⁵⁵-substituted or unsubstituted aryl (e.g., C₆-C₁₀ aryl, C₁₀ aryl, or phenyl). In embodiments, R⁵⁴ is R⁵⁵-substituted aryl (e.g., C₆-C₁₀ aryl, C₁₀ aryl, or phenyl). In embodiments, R⁵⁴ is an unsubstituted aryl (e.g., C₆-C₁₀ aryl, C₁₀ aryl, or phenyl). In embodiments, R⁵⁴ is R⁵⁵-substituted or unsubstituted heteroaryl (e.g., 5 to 10 membered heteroaryl, 5 to 9 membered heteroaryl, or 5 to 6 membered heteroaryl). In embodiments, R⁵⁴ is R⁵⁵-substituted heteroaryl (e.g., 5 to 10 membered heteroaryl, 5 to 9 membered heteroaryl, or 5 to 6 membered heteroaryl). In embodiments, R⁵⁴ is an unsubstituted heteroaryl (e.g., 5 to 10 membered heteroaryl, 5 to 9 membered heteroaryl, or 5 to 6 membered heteroaryl).

In embodiments, R^{27A} is hydrogen, halogen, —CF₃, —CCl₃, —CBr₃, —CI₃, —COOH, —CONH₂, R⁵³-substituted or unsubstituted alkyl (e.g., C₁-C₈ alkyl, C₁-C₆ alkyl, or C₁-C₄ alkyl), R⁵³-substituted or unsubstituted heteroalkyl (e.g., 2 to 8 membered heteroalkyl, 2 to 6 membered heteroalkyl, or 2 to 4 membered heteroalkyl), R⁵³-substituted or unsubstituted cycloalkyl (e.g., C₃-C₈ cycloalkyl, C₃-C₆ cycloalkyl, or C₅-C₆ cycloalkyl), R⁵³-substituted or unsubstituted heterocycloalkyl (e.g., 3 to 8 membered heterocycloalkyl, 3 to 6 membered heterocycloalkyl, or 5 to 6 membered heterocycloalkyl), R⁵³-substituted or unsubstituted aryl (e.g., C₆-C₁₀ aryl, C₁₀ aryl, or phenyl), or R⁵³-substituted or unsubstituted heteroaryl (e.g., 5 to 10 membered heteroaryl, 5 to 9 membered heteroaryl, or 5 to 6 membered heteroaryl).

In embodiments, R^{27B} is hydrogen, halogen, —CF₃, —CCl₃, —CBr₃, —CI₃, —COOH, —CONH₂, R⁵³-substituted or unsubstituted alkyl (e.g., C₁-C₈ alkyl, C₁-C₆ alkyl, or C₁-C₄ alkyl), R⁵³-substituted or unsubstituted heteroalkyl (e.g., 2 to 8 membered heteroalkyl, 2 to 6 membered heteroalkyl, or 2 to 4 membered heteroalkyl), R⁵³-substituted or unsubstituted cycloalkyl (e.g., C₃-C₈ cycloalkyl, C₃-C₆ cycloalkyl, or C₅-C₆ cycloalkyl), R⁵³-substituted or unsubstituted heterocycloalkyl (e.g., 3 to 8 membered heterocycloalkyl, 3 to 6 membered heterocycloalkyl, or 5 to 6 membered heterocycloalkyl), R⁵³-substituted or unsubstituted aryl (e.g., C₆-C₁₀ aryl, C₁₀ aryl, or phenyl), or R⁵³-substituted or unsubstituted heteroaryl (e.g., 5 to 10 membered heteroaryl, 5 to 9 membered heteroaryl, or 5 to 6 membered heteroaryl). In embodiments, R^{27B} and R^{27C} substituents bonded to the same nitrogen atom may optionally be joined to form a R⁵³⁻substituted or unsubstituted heterocycloalkyl (e.g., 3 to 8 membered heterocycloalkyl, 3 to 6 membered heterocycloalkyl, or 5 to 6 membered heterocycloalkyl) or R⁵³-substituted or unsubstituted heteroaryl (e.g., 5 to 10 membered heteroaryl, 5 to 9 membered heteroaryl, or 5 to 6 membered heteroaryl).

In embodiments, R^{27C} is hydrogen, halogen, —CF₃, —CCl₃, —CBr₃, —CI₃, —COOH, —CONH₂, R⁵³-substituted or unsubstituted alkyl (e.g., C₁-C₈ alkyl, C₁-C₆ alkyl, or C₁-C₄ alkyl), R⁵³-substituted or unsubstituted heteroalkyl (e.g., 2 to 8 membered heteroalkyl, 2 to 6 membered heteroalkyl, or 2 to 4 membered heteroalkyl), R⁵³-substituted or unsubstituted cycloalkyl (e.g., C₃-C₈ cycloalkyl, C₃-C₆ cycloalkyl, or C₅-C₆ cycloalkyl), R⁵³-substituted or unsubstituted heterocycloalkyl (e.g., 3 to 8 membered heterocycloalkyl, 3 to 6 membered heterocycloalkyl, or 5 to 6 membered heterocycloalkyl), R⁵³-substituted or unsubstituted aryl (e.g., C₆-C₁₀ aryl, C₁₀ aryl, or phenyl), or R⁵³-substituted or unsubstituted heteroaryl (e.g., 5 to 10 membered heteroaryl, 5 to 9 membered heteroaryl, or 5 to 6 membered heteroaryl). In embodiments, R^{27B} and R^{27C} substituents bonded to the same nitrogen atom may optionally be joined to form a R⁵³⁻substituted or unsubstituted heterocycloalkyl (e.g., 3 to 8 membered heterocycloalkyl, 3 to 6 membered heterocycloalkyl, or 5 to 6 membered heterocycloalkyl) or R⁵³-substituted or unsubstituted heteroaryl (e.g., 5 to 10 membered heteroaryl, 5 to 9 membered heteroaryl, or 5 to 6 membered heteroaryl).

In embodiments, R^{27D} is hydrogen, halogen, —CF₃, —CCl₃, —CBr₃, —CI₃, —COOH, —CONH₂, R⁵³-substituted or unsubstituted alkyl (e.g., C₁-C₈ alkyl, C₁-C₆ alkyl, or C₁-C₄ alkyl), R⁵³-substituted or unsubstituted heteroalkyl (e.g., 2 to 8 membered heteroalkyl, 2 to 6 membered heteroalkyl, or 2 to 4 membered heteroalkyl), R⁵³-substituted or unsubstituted cycloalkyl (e.g., C₃-C₈ cycloalkyl, C₃-C₆ cycloalkyl, or C₅-C₆ cycloalkyl), R⁵³-substituted or unsubstituted heterocycloalkyl (e.g., 3 to 8 membered heterocycloalkyl, 3 to 6 membered heterocycloalkyl, or 5 to 6 membered heterocycloalkyl), R⁵³-substituted or unsubstituted aryl (e.g., C₆-C₁₀ aryl, C₁₀ aryl, or phenyl), or R⁵³-substituted or unsubstituted heteroaryl (e.g., 5 to 10 membered heteroaryl, 5 to 9 membered heteroaryl, or 5 to 6 membered heteroaryl).

In embodiments, R²⁷ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R²⁷ is hydrogen, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R²⁴, R²⁵, R²⁶ and R²⁷ are independently —CH₂OH, —C(O)NH₂, —C(O)NHCH₃, —CH(OH)CH₂CH₃ or —CH(OH)CH₃.

In embodiments, R²⁸ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₈H, —SOᵥ₂₈NH₂, —NHNH₂, —ONH₂, —NHC(O)NHNH₂, —NHC(O)NH₂, —N(O)ₘ₂₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). Two adjacent R²⁸ substituents may combine to form substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, -Ring A-(R²⁸)z5 is:

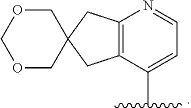 [SMILES: *c1ccnc2c1CC1(COCOC1)C2]

In embodiments, R³³ is hydrogen. In embodiments, R³³ is halogen. In embodiments, R³³ is —CF₃. In embodiments, R³³ is —CCl₃. In embodiments, R³³ is —CBr₃. In embodiments, R³³ is —Cl₃. In embodiments, R³³ is —CHF₂. In embodiments, R³³ is —CHCl₂. In embodiments, R³³ is —CHBr₂. In embodiments, R³³ is —CHI₂. In embodiments, R³³ is —CH₂F. In embodiments, R³³ is —CH₂Cl. In embodiments, R³³ is —CH₂Br. In embodiments, R³³ is —CH₂I. In embodiments, R³³ is —CN. In embodiments, R³³ is —SOₙ₃₃H. In embodiments, R³³ is —SO₃₃NH₂. In embodiments, R³³ is —C(O)H. In embodiments, R³³ is —C(O)OH. In embodiments, R³³ is —C(O)NH₂. In embodiments, R³³ is —OCF₃. In embodiments, R³³ is —OCCl₃. In embodiments, R³³ is —OCBr₃. In embodiments, R³³ is —OCl₃. In embodiments, R³³ is —OCHF₂. In embodiments, R³³ is —OCHCl₂. In embodiments, R³³ is —OCHBr₂. In embodiments, R³³ is —OCHI₂. In embodiments, R³³ is —N(CH₃)₂. In embodiments, R³³ is —NH(CH₃). In embodiments, R³³ is independently —OCH₂F. In embodiments, R³³ is independently —OCH₂Cl. In embodiments, R³³ is independently —OCH₂Br. In embodiments, R³³ is independently —OCH₂I.

In embodiments, when R³³ is attached to nitrogen, R³³ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃₃H, —SOᵥ₃₃NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³³ is hydrogen. In embodiments, when R³³ is attached to nitrogen, R³³ is hydrogen. In embodiments, when R³³ is attached to nitrogen, R³³ is halogen. In embodiments, when R³³ is attached to nitrogen, R³³ is —CF₃. In embodiments, when R³³ is attached to nitrogen, R³³ is —CCl₃. In embodiments, when R³³ is attached to nitrogen, R³³ is —CBr₃. In embodiments, when R³³ is attached to nitrogen, R³³ is —Cl₃. In embodiments, when R³³ is attached to nitrogen, R³³ is —CHF₂. In embodiments, when R³³ is attached to nitrogen, R³³ is —CHCl₂. In embodiments, when R³³ is attached to nitrogen, R³³ is —CHBr₂. In embodiments, when R³³ is attached to nitrogen, R³³ is —CHI₂. In embodiments, when R³³ is attached to nitrogen, R³³ is —CH₂F. In embodiments, when R³³ is attached to nitrogen, R³³ is —CH₂Cl. In embodiments, when R³³ is attached to nitrogen, R³³ is —CH₂Br. In embodiments, when R³³ is attached to nitrogen, R³³ is —CH₂I. In embodiments, when R³³ is attached to nitrogen, R³³ is —CN. In embodiments, when R³³ is attached to nitrogen, R³³ is —SOₙ₃₃H. In embodiments, when R³³ is attached to nitrogen, R³³ is —SOᵥ₃₃NH₂. In embodiments, when R³³ is attached to nitrogen, R³³ is —C(O)H. In embodiments, when R³³ is attached to nitrogen, R³³ is —C(O)OH. In embodiments, when R³³ is attached to nitrogen, R³³ is —C(O)NH₂. In embodiments, when R³³ is attached to nitrogen, R³³ is —OHOCF₃. In embodiments, when R³³ is attached to nitrogen, R³³ is —OCCl₃. In embodiments, when R³³ is attached to nitrogen, R³³ is —OCBr₃. In embodiments, when R³³ is attached to nitrogen, R³³ is —OCl₃. In embodiments, when R³³ is attached to nitrogen, R³³ is —OCHF₂. In embodiments, when R³³ is attached to nitrogen, R³³ is —OCHCl₂. In embodiments, when R³³ is attached to nitrogen, R³³ is —OCHBr₂. In embodiments, when R³³ is attached to nitrogen, R³³ is —OCHI₂. In embodiments, when R³³ is attached to nitrogen, R³³ is —N(CH₃)₂. In embodiments, when R³³ is attached to nitrogen, R³³ is —NH(CH₃). In embodiments, when R³³ is attached to nitrogen, R³³ is —OCH₂F. In embodiments, when R³³ is attached to nitrogen, R³³ is —OCH₂Cl. In embodiments, when R³³ is attached to nitrogen, R²¹ is —OCH₂Br. In embodiments, when R²¹ is attached to nitrogen, R²¹ is —OCH₂I.

In embodiments, R³³ is a substituted or unsubstituted alkyl (e.g., C₁-C₈ alkyl, C₁-C₆ alkyl, or C₁-C₄ alkyl). In embodiments, R³³ is a substituted alkyl (e.g., C₁-C₈ alkyl, C₁-C₆ alkyl, or C₁-C₄ alkyl). In embodiments, R³³ is an unsubstituted alkyl (e.g., C₁-C₈ alkyl, C₁-C₆ alkyl, or C₁-C₄ alkyl). In embodiments, R³³ is a substituted or unsubstituted heteroalkyl (e.g., 2 to 8 membered heteroalkyl, 2 to 6 membered heteroalkyl, or 2 to 4 membered heteroalkyl). In embodiments, R³³ is a substituted heteroalkyl (e.g., 2 to 8 membered heteroalkyl, 2 to 6 membered heteroalkyl, or 2 to 4 membered heteroalkyl). In embodiments, R³³ is an unsubstituted heteroalkyl (e.g., 2 to 8 membered heteroalkyl, 2 to 6 membered heteroalkyl, or 2 to 4 membered heteroalkyl). In embodiments, R³³ is a substituted or unsubstituted cycloalkyl (e.g., C₃-C₈ cycloalkyl, C₃-C₆ cycloalkyl, or C₅-C₆ cycloalkyl). In embodiments, R³³ is a substituted cycloalkyl (e.g., C₃-C₈ cycloalkyl, C₃-C₆ cycloalkyl, or C₅-C₆ cycloalkyl). In embodiments, R³³ is an unsubstituted cycloalkyl (e.g., C₃-C₈ cycloalkyl, C₃-C₆ cycloalkyl, or C₅-C₆ cycloalkyl). In embodiments, R³³ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³³ is a substituted heterocycloalkyl (e.g., 3 to 8 membered heterocycloalkyl, 3 to 6 membered heterocycloalkyl, or 5 to 6 membered heterocycloalkyl). In embodiments, R³³ is an unsubstituted heterocycloalkyl (e.g., 3 to 8 membered heterocycloalkyl, 3 to 6 membered heterocycloalkyl, or 5 to 6 membered heterocycloalkyl). In embodiments, R³³ is a substituted or unsubstituted aryl (e.g., C₆-C₁₀ aryl, C₁₀ aryl, or phenyl). In embodiments, R³³ is a substituted aryl (e.g., C₆-C₁₀ aryl, C₁₀ aryl, or phenyl). In embodiments, R³³ is an unsubstituted aryl (e.g., C₆-C₁₀ aryl, C₁₀ aryl, or phenyl). In embodiments, R³³ is a substituted or unsubstituted heteroaryl (e.g., 5 to 10 membered heteroaryl, 5 to 9 membered heteroaryl, or 5 to 6 membered heteroaryl). In embodiments, R³³ is a substituted heteroaryl (e.g., 5 to 10 membered heteroaryl, 5 to 9 membered heteroaryl, or 5 to 6 membered heteroaryl). In embodiments, R³³ is an unsubstituted heteroaryl (e.g., 5 to 10 membered heteroaryl, 5 to 9 membered heteroaryl, or 5 to 6 membered heteroaryl).

In embodiments, when R³³ is attached to nitrogen, R³³ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃₃H, —SOᵥ₃₃NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³³ is hydrogen. In embodiments, R³³ is substituted C₁-C₄ alkyl. In embodiments, R³³ is unsubstituted benzyl

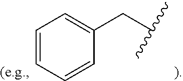 [SMILES: *Cc1ccccc1]

In embodiments, R⁵⁹ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₅₉H, —SOᵥ₅₉NH₂, —NHNH₂, —ONH₂, —NHC(O)NHNH₂, —NHC(O)NH₂, —N(O)ₘ₅₉, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, two R⁵⁹ substituents bonded to adjacent carbon atoms may optionally be joined to form a substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R⁵⁹ is independently halogen. In embodiments, R⁵⁹ is independently oxo. In embodiments, R⁵⁹ is independently —CF₃. In embodiments, R⁵⁹ is independently —CCl₃. In embodiments, R⁵⁹ is independently —CBr₃. In embodiments, R⁵⁹ is independently —Cl₃. In embodiments, R⁵⁹ is independently —CHF₂. In embodiments, R⁵⁹ is independently —CHCl₂. In embodiments, R⁵⁹ is independently —CHBr₂. In embodiments, R⁵⁹ is independently —CHI₂. In embodiments, R⁵⁹ is independently —CH₂F. In embodiments, R⁵⁹ is independently —CH₂Cl. In embodiments, R⁵⁹ is independently —CH₂Br. In embodiments, R⁵⁹ is independently —CH₂I. In embodiments, R⁵⁹ is independently —CN. In embodiments, R⁵⁹ is independently —N₃. In embodiments, R⁵⁹ is independently —SOₙ₅₉H. In embodiments, R⁵⁹ is independently —SOᵥ₅₉NH₂. In embodiments, R⁵⁹ is independently —NHNH₂. In embodiments, R⁵⁹ is independently —ONH₂. In embodiments, R⁵⁹ is independently —NHC(O)NHNH₂. In embodiments, R⁵⁹ is independently —NHC(O)NH₂. In embodiments, R⁵⁹ is independently —N(O)ₘ₅₉. In embodiments, R⁵⁹ is independently —NH₂. In embodiments, R⁵⁹ is independently —C(O)H. In embodiments, R⁵⁹ is independently —C(O)OH. In embodiments, R⁵⁹ is independently —C(O)NH₂. In embodiments, R⁵⁹ is independently —OH. In embodiments, R⁵⁹ is independently —NHSO₂H. In embodiments, R⁵⁹ is independently —NHC(O)H. In embodiments, R⁵⁹ is independently —NHC(O)OH. In embodiments, R⁵⁹ is independently —NHOH. In embodiments, R⁵⁹ is independently —OCF₃. In embodiments, R⁵⁹ is independently —OCCl₃. In embodiments, R⁵⁹ is independently —OCBr₃. In embodiments, R⁵⁹ is independently —OCl₃. In embodiments, R⁵⁹ is independently —OCHF₂. In embodiments, R⁵⁹ is independently —OCHCl₂. In embodiments, R⁵⁹ is independently —OCHBr₂. In embodiments, R⁵⁹ is independently —OCHI₂. In embodiments, R⁵⁹ is independently —OCH₂F. In embodiments, R⁵⁹ is independently —OCH₂Cl. In embodiments, R⁵⁹ is independently —OCH₂Br. In embodiments, R⁵⁹ is independently —OCH₂I.

In embodiments, R⁵⁹ is independently a substituted or unsubstituted alkyl (e.g., C₁-C₈ alkyl, C₁-C₆ alkyl, or C₁-C₄ alkyl). In embodiments, R⁵⁹ is independently a substituted alkyl (e.g., C₁-C₈ alkyl, C₁-C₆ alkyl, or C₁-C₄ alkyl). In embodiments, R⁵⁹ is independently an unsubstituted alkyl (e.g., C₁-C₈ alkyl, C₁-C₆ alkyl, or C₁-C₄ alkyl). In embodiments, R⁵⁹ is independently a substituted or unsubstituted heteroalkyl (e.g., 2 to 8 membered heteroalkyl, 2 to 6 membered heteroalkyl, or 2 to 4 membered heteroalkyl). In embodiments, R⁵⁹ is independently a substituted heteroalkyl (e.g., 2 to 8 membered heteroalkyl, 2 to 6 membered heteroalkyl, or 2 to 4 membered heteroalkyl). In embodiments, R⁵⁹ is independently an unsubstituted heteroalkyl (e.g., 2 to 8 membered heteroalkyl, 2 to 6 membered heteroalkyl, or 2 to 4 membered heteroalkyl). In embodiments, R⁵⁹ is independently a substituted or unsubstituted cycloalkyl (e.g., C₃-C₈ cycloalkyl, C₃-C₆ cycloalkyl, or C₅-C₆ cycloalkyl). In embodiments, R⁵⁹ is independently a substituted cycloalkyl (e.g., C₃-C₈ cycloalkyl, C₃-C₆ cycloalkyl, or C₅-C₆ cycloalkyl). In embodiments, R⁵⁹ is independently an unsubstituted cycloalkyl (e.g., C₃-C₈ cycloalkyl, C₃-C₆ cycloalkyl, or C₅-C₆ cycloalkyl). In embodiments, R⁵⁹ is independently a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁵⁹ is independently a substituted heterocycloalkyl (e.g., 3 to 8 membered heterocycloalkyl, 3 to 6 membered heterocycloalkyl, or 5 to 6 membered heterocycloalkyl). In embodiments, R⁵⁹ is independently an unsubstituted heterocycloalkyl (e.g., 3 to 8 membered heterocycloalkyl, 3 to 6 membered heterocycloalkyl, or 5 to 6 membered heterocycloalkyl). In embodiments, R⁵⁹ is independently a substituted or unsubstituted aryl (e.g., C₆-C₁₀ aryl, C₁₀ aryl, or phenyl). In embodiments, R⁵⁹ is independently a substituted aryl (e.g., C₆-C₁₀ aryl, C₁₀ aryl, or phenyl). In embodiments, R⁵⁹ is independently an unsubstituted aryl (e.g., C₆-C₁₀ aryl, C₁₀ aryl, or phenyl). In embodiments, R⁵⁹ is independently a substituted or unsubstituted heteroaryl (e.g., 5 to 10 membered heteroaryl, 5 to 9 membered heteroaryl, or 5 to 6 membered heteroaryl). In embodiments, R⁵⁹ is independently a substituted heteroaryl (e.g., 5 to 10 membered heteroaryl, 5 to 9 membered heteroaryl, or 5 to 6 membered heteroaryl). In embodiments, R⁵⁹ is independently an unsubstituted heteroaryl (e.g., 5 to 10 membered heteroaryl, 5 to 9 membered heteroaryl, or 5 to 6 membered heteroaryl).

In embodiments, two R⁵⁹ substituents bonded to adjacent carbon atoms may optionally be joined to form a substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, two R⁵⁹ substituents bonded to adjacent carbon atoms are joined to form a 5-membered substituted or unsubstituted heteroaryl or a 6-membered substituted or unsubstituted aryl. In embodiments, two R⁵⁹ substituents bonded to adjacent carbon atoms may optionally be joined to form a substituted cycloalkyl (e.g., C₃-C₈ cycloalkyl, C₃-C₆ cycloalkyl, or C₅-C₆ cycloalkyl), substituted heterocycloalkyl (e.g., 3 to 8 membered heterocycloalkyl, 3 to 6 membered heterocycloalkyl, or 5 to 6 membered heterocycloalkyl), substituted aryl (e.g., C₆-C₁₀ aryl, C₁₀ aryl, or phenyl), or substituted heteroaryl (e.g., 5 to 10 membered heteroaryl, 5 to 9 membered heteroaryl, or 5 to 6 membered heteroaryl). In embodiments, two R⁵⁹ substituents bonded to adjacent carbon atoms may optionally be joined to form an unsubstituted cycloalkyl (e.g., C₃-C₈ cycloalkyl, C₃-C₆ cycloalkyl, or C₅-C₆ cycloalkyl), an unsubstituted heterocycloalkyl (e.g., 3 to 8 membered heterocycloalkyl, 3 to 6 membered heterocycloalkyl, or 5 to 6 membered heterocycloalkyl), an unsubstituted aryl (e.g., C₆-C₁₀ aryl, C₁₀ aryl, or phenyl), or an unsubstituted heteroaryl (e.g., 5 to 10 membered heteroaryl, 5 to 9 membered heteroaryl, or 5 to 6 membered heteroaryl).

In embodiments, two R⁵⁹ substituents bonded to adjacent carbon atoms may optionally be joined to form a substituted cycloalkyl (e.g., C₃-C₈ cycloalkyl, C₃-C₆ cycloalkyl, or C₅-C₆ cycloalkyl). In embodiments, two R⁵⁹ substituents bonded to adjacent carbon atoms may optionally be joined to form a substituted heterocycloalkyl (e.g., 3 to 8 membered heterocycloalkyl, 3 to 6 membered heterocycloalkyl, or 5 to 6 membered heterocycloalkyl). In embodiments, two R⁵⁹ substituents bonded to adjacent carbon atoms may optionally be joined to form a substituted aryl (e.g., C₆-C₁₀ aryl, C₁₀ aryl, or phenyl). In embodiments, two R⁵⁹ substituents bonded to adjacent carbon atoms may optionally be joined to form a substituted heteroaryl (e.g., 5 to 10 membered heteroaryl, 5 to 9 membered heteroaryl, or 5 to 6 membered heteroaryl).

In embodiments, two R⁵⁹ substituents bonded to adjacent carbon atoms may optionally be joined to form an unsubstituted cycloalkyl (e.g., C₃-C₈ cycloalkyl, C₃-C₆ cycloalkyl, or C₅-C₆ cycloalkyl). In embodiments, two R⁵⁹ substituents bonded to adjacent carbon atoms may optionally be joined to form an unsubstituted heterocycloalkyl (e.g., 3 to 8 membered heterocycloalkyl, 3 to 6 membered heterocycloalkyl, or 5 to 6 membered heterocycloalkyl). In embodiments, two R⁵⁹ substituents bonded to adjacent carbon atoms may optionally be joined to form an unsubstituted aryl. In embodiments, two R⁵⁹ substituents bonded to adjacent carbon atoms may optionally be joined to form an unsubstituted heteroaryl (e.g., 5 to 10 membered heteroaryl, 5 to 9 membered heteroaryl, or 5 to 6 membered heteroaryl).

In embodiments, R⁶⁰ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₆₀H, —SOᵥ₆₀NH₂, —NHNH₂, —ONH₂, —NHC(O)NHNH₂, —NHC(O)NH₂, —N(O)ₘ₆₀, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, two R⁵⁹ substituents bonded to adjacent carbon atoms may optionally be joined to form a substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R⁶⁰ is independently halogen. In embodiments, R⁶⁰ is independently oxo. In embodiments, R⁶⁰ is independently —CF₃. In embodiments, R⁶⁰ is independently —CCl₃. In embodiments, R⁶⁰ is independently —CBr₃. In embodiments, R⁶⁰ is independently —Cl₃. In embodiments, R⁶⁰ is independently —CHF₂. In embodiments, R⁶⁰ is independently —CHCl₂. In embodiments, R⁶⁰ is independently —CHBr₂. In embodiments, R⁶⁰ is independently —CHI₂. In embodiments, R⁶⁰ is independently —CH₂F. In embodiments, R⁶⁰ is independently —CH₂Cl. In embodiments, R⁶⁰ is independently —CH₂Br. In embodiments, R⁶⁰ is independently —CH₂I. In embodiments, R⁶⁰ is independently —CN. In embodiments, R⁶⁰ is independently —N₃. In embodiments, R⁶⁰ is independently —SOₙ₆₀H. In embodiments, R⁶⁰ is independently —SOᵥ₆₀NH₂. In embodiments, R⁶⁰ is independently —NHNH₂. In embodiments, R⁶⁰ is independently —ONH₂. In embodiments, R⁶⁰ is independently —NHC(O)NHNH₂. In embodiments, R⁶⁰ is independently —NHC(O)NH₂. In embodiments, R⁶⁰ is independently —N(O)ₘ₆₀. In embodiments, R⁶⁰ is independently —NH₂. In embodiments, R⁵⁹ is independently —C(O)H. In embodiments, R⁶⁰ is independently —C(O)OH. In embodiments, R⁵⁹ is independently —C(O)NH₂. In embodiments, R⁶⁰ is independently —OH. In embodiments, R⁶⁰ is independently —NHSO₂H. In embodiments, R⁶⁰ is independently —NHC(O)H. In embodiments, R⁶⁰ is independently —NHC(O)OH. In embodiments, R⁶⁰ is independently —NHOH. In embodiments, R⁶⁰ is independently —OCF₃. In embodiments, R⁶⁰ is independently —OCCl₃. In embodiments, R⁶⁰ is independently —OCBr₃. In embodiments, R⁶⁰ is independently —OCl₃. In embodiments, R⁶⁰ is independently —OCHF₂. In embodiments, R⁶⁰ is independently —OCHCl₂. In embodiments, R⁶⁰ is independently —OCHBr₂. In embodiments, R⁶⁰ is independently —OCHI₂. In embodiments, R⁶⁰ is independently —OCH₂F. In embodiments, R⁶⁰ is independently —OCH₂Cl. In embodiments, R⁶⁰ is independently —OCH₂Br. In embodiments, R⁶⁰ is independently —OCH₂I.

In embodiments, R⁶⁰ is independently a substituted or unsubstituted alkyl (e.g., C₁-C₈ alkyl, C₁-C₆ alkyl, or C₁-C₄ alkyl). In embodiments, R⁶⁰ is independently a substituted alkyl (e.g., C₁-C₈ alkyl, C₁-C₆ alkyl, or C₁-C₄ alkyl). In embodiments, R⁶⁰ is independently an unsubstituted alkyl (e.g., C₁-C₈ alkyl, C₁-C₆ alkyl, or C₁-C₄ alkyl). In embodiments, R⁶⁰ is independently a substituted or unsubstituted heteroalkyl (e.g., 2 to 8 membered heteroalkyl, 2 to 6 membered heteroalkyl, or 2 to 4 membered heteroalkyl). In embodiments, R⁶⁰ is independently a substituted heteroalkyl (e.g., 2 to 8 membered heteroalkyl, 2 to 6 membered heteroalkyl, or 2 to 4 membered heteroalkyl). In embodiments, R⁶⁰ is independently an unsubstituted heteroalkyl (e.g., 2 to 8 membered heteroalkyl, 2 to 6 membered heteroalkyl, or 2 to 4 membered heteroalkyl). In embodiments, R⁶⁰ is independently a substituted or unsubstituted cycloalkyl (e.g., C₃-C₈ cycloalkyl, C₃-C₆ cycloalkyl, or C₅-C₆ cycloalkyl). In embodiments, R⁶⁰ is independently a substituted cycloalkyl (e.g., C₃-C₈ cycloalkyl, C₃-C₆ cycloalkyl, or C₅-C₆ cycloalkyl). In embodiments, R⁶⁰ is independently an unsubstituted cycloalkyl (e.g., C₃-C₈ cycloalkyl, C₃-C₆ cycloalkyl, or C₅-C₆ cycloalkyl). In embodiments, R⁶⁰ is independently a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁶⁰ is independently a substituted heterocycloalkyl (e.g., 3 to 8 membered heterocycloalkyl, 3 to 6 membered heterocycloalkyl, or 5 to 6 membered heterocycloalkyl). In embodiments, R⁶⁰ is independently an unsubstituted heterocycloalkyl (e.g., 3 to 8 membered heterocycloalkyl, 3 to 6 membered heterocycloalkyl, or 5 to 6 membered heterocycloalkyl). In embodiments, R⁶⁰ is independently a substituted or unsubstituted aryl (e.g., C₆-C₁₀ aryl, C₁₀ aryl, or phenyl). In embodiments, R⁶⁰ is independently a substituted aryl (e.g., C₆-C₁₀ aryl, C₁₀ aryl, or phenyl). In embodiments, R⁶⁰ is independently an unsubstituted aryl (e.g., C₆-C₁₀ aryl, C₁₀ aryl, or phenyl). In embodiments, R⁶⁰ is independently a substituted or unsubstituted heteroaryl (e.g., 5 to 10 membered heteroaryl, 5 to 9 membered heteroaryl, or 5 to 6 membered heteroaryl). In embodiments, R⁶⁰ is independently a substituted heteroaryl (e.g., 5 to 10 membered heteroaryl, 5 to 9 membered heteroaryl, or 5 to 6 membered heteroaryl). In embodiments, R⁶⁰ is independently an unsubstituted heteroaryl (e.g., 5 to 10 membered heteroaryl, 5 to 9 membered heteroaryl, or 5 to 6 membered heteroaryl).

In embodiments, two R⁶⁰ substituents bonded to adjacent carbon atoms may optionally be joined to form a substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, two R⁶⁰ substituents bonded to adjacent carbon atoms are joined to form a 5-membered substituted or unsubstituted heteroaryl or a 6-membered substituted or unsubstituted aryl. In embodiments, two R⁶⁰ substituents bonded to adjacent carbon atoms may optionally be joined to form a substituted cycloalkyl (e.g., C₃-C₈ cycloalkyl, C₃-C₆ cycloalkyl, or C₅-C₆ cycloalkyl), substituted heterocycloalkyl (e.g., 3 to 8 membered heterocycloalkyl, 3 to 6 membered heterocycloalkyl, or 5 to 6 membered heterocycloalkyl), substituted aryl (e.g., C₆-C₁₀ aryl, C₁₀ aryl, or phenyl), or substituted heteroaryl (e.g., 5 to 10 membered heteroaryl, 5 to 9 membered heteroaryl, or 5 to 6 membered heteroaryl). In embodiments, two R⁶⁰ substituents bonded to adjacent carbon atoms may optionally be joined to form an unsubstituted cycloalkyl (e.g., C₃-C₈ cycloalkyl, C₃-C₆ cycloalkyl, or C₅-C₆ cycloalkyl), an unsubstituted heterocycloalkyl (e.g., 3 to 8 membered heterocycloalkyl, 3 to 6 membered heterocycloalkyl, or 5 to 6 membered heterocycloalkyl), an unsubstituted aryl (e.g., C₆-C₁₀ aryl, C₁₀ aryl, or phenyl), or an unsubstituted heteroaryl (e.g., 5 to 10 membered heteroaryl, 5 to 9 membered heteroaryl, or 5 to 6 membered heteroaryl).

In embodiments, two R⁶⁰ substituents bonded to adjacent carbon atoms may optionally be joined to form a substituted cycloalkyl (e.g., C₃-C₈ cycloalkyl, C₃-C₆ cycloalkyl, or C₅-C₆ cycloalkyl). In embodiments, two R⁶⁰ substituents bonded to adjacent carbon atoms may optionally be joined to form a substituted heterocycloalkyl (e.g., 3 to 8 membered heterocycloalkyl, 3 to 6 membered heterocycloalkyl, or 5 to 6 membered heterocycloalkyl). In embodiments, two R⁶⁰ substituents bonded to adjacent carbon atoms may optionally be joined to form a substituted aryl (e.g., C₆-C₁₀ aryl, C₁₀ aryl, or phenyl). In embodiments, two R⁶⁰ substituents bonded to adjacent carbon atoms may optionally be joined to form a substituted heteroaryl (e.g., 5 to 10 membered heteroaryl, 5 to 9 membered heteroaryl, or 5 to 6 membered heteroaryl).

In embodiments, two R⁶⁰ substituents bonded to adjacent carbon atoms may optionally be joined to form an unsubstituted cycloalkyl (e.g., C₃-C₈ cycloalkyl, C₃-C₆ cycloalkyl, or C₅-C₆ cycloalkyl). In embodiments, two R⁶⁰ substituents bonded to adjacent carbon atoms may optionally be joined to form an unsubstituted heterocycloalkyl (e.g., 3 to 8 membered heterocycloalkyl, 3 to 6 membered heterocycloalkyl, or 5 to 6 membered heterocycloalkyl). In embodiments, two R⁶⁰ substituents bonded to adjacent carbon atoms may optionally be joined to form an unsubstituted aryl. In embodiments, two R⁶⁰ substituents bonded to adjacent carbon atoms may optionally be joined to form an unsubstituted heteroaryl (e.g., 5 to 10 membered heteroaryl, 5 to 9 membered heteroaryl, or 5 to 6 membered heteroaryl).

In embodiments, R⁶¹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₆₁R^{61A}, —SOᵥ₆₁NR^{61B}R^{61C}, —NHNR^{61B}R^{61C}, —ONR^{61B}R^{61C}, —NHC(O)NHNR^{61B}R^{61C}, —NHC(O)NR^{61B}R^{61C}, —N(O)ₘ₆₁, —NR^{61B}R^{61C}, —C(O)R^{61D}, —C(O)OR^{61D}, —C(O)NR^{61B}R^{61C}, —OR^{61A}, —NR^{61B}SO₂R^{61A}—NR^{61B}C(O)R^{61D}, —NR^{61B}C(O)OR^{61D}, —NR^{61B}OR^{61D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R⁶¹ is a substituted or unsubstituted alkyl (e.g., C₁-C₈ alkyl, C₁-C₆ alkyl, or C₁-C₄ alkyl). In embodiments, R⁶¹ is a substituted alkyl (e.g., C₁-C₈ alkyl, C₁-C₆ alkyl, or C₁-C₄ alkyl). In embodiments, R⁶¹ is an unsubstituted alkyl (e.g., C₁-C₈ alkyl, C₁-C₆ alkyl, or C₁-C₄ alkyl). In embodiments, R⁶¹ is a substituted or unsubstituted heteroalkyl (e.g., 2 to 8 membered heteroalkyl, 2 to 6 membered heteroalkyl, or 2 to 4 membered heteroalkyl). In embodiments, R⁶¹ is a substituted heteroalkyl (e.g., 2 to 8 membered heteroalkyl, 2 to 6 membered heteroalkyl, or 2 to 4 membered heteroalkyl). In embodiments, R⁶¹ is an unsubstituted heteroalkyl (e.g., 2 to 8 membered heteroalkyl, 2 to 6 membered heteroalkyl, or 2 to 4 membered heteroalkyl). In embodiments, R⁶¹ is a substituted or unsubstituted cycloalkyl (e.g., C₃-C₈ cycloalkyl, C₃-C₆ cycloalkyl, or C₅-C₆ cycloalkyl). In embodiments, R⁶¹ is a substituted cycloalkyl (e.g., C₃-C₈ cycloalkyl, C₃-C₆ cycloalkyl, or C₅-C₆ cycloalkyl). In embodiments, R⁶¹ is an unsubstituted cycloalkyl (e.g., C₃-C₈ cycloalkyl, C₃-C₆ cycloalkyl, or C₅-C₆ cycloalkyl). In embodiments, R⁶¹ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁶¹ is a substituted heterocycloalkyl (e.g., 3 to 8 membered heterocycloalkyl, 3 to 6 membered heterocycloalkyl, or 5 to 6 membered heterocycloalkyl). In embodiments, R⁶¹ is an unsubstituted heterocycloalkyl (e.g., 3 to 8 membered heterocycloalkyl, 3 to 6 membered heterocycloalkyl, or 5 to 6 membered heterocycloalkyl). In embodiments, R⁶¹ is a substituted or unsubstituted aryl (e.g., C₆-C₁₀ aryl, C₁₀ aryl, or phenyl). In embodiments, R⁶¹ is a substituted aryl (e.g., C₆-C₁₀ aryl, C₁₀ aryl, or phenyl). In embodiments, R⁶¹ is an unsubstituted aryl (e.g., C₆-C₁₀ aryl, C₁₀ aryl, or phenyl). In embodiments, R⁶¹ is a substituted or unsubstituted heteroaryl (e.g., 5 to 10 membered heteroaryl, 5 to 9 membered heteroaryl, or 5 to 6 membered heteroaryl). In embodiments, R⁶¹ is a substituted heteroaryl (e.g., 5 to 10 membered heteroaryl, 5 to 9 membered heteroaryl, or 5 to 6 membered heteroaryl). In embodiments, R⁶¹ is an unsubstituted heteroaryl (e.g., 5 to 10 membered heteroaryl, 5 to 9 membered heteroaryl, or 5 to 6 membered heteroaryl).

In embodiments, R⁶² is independently hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₆₂H, —SOᵥ₆₂NH₂, —NHNH₂, —ONH₂, —NHC(O)NHNH₂, —NHC(O)NH₂, —N(O)ₘ₆₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R⁶² is a substituted or unsubstituted alkyl (e.g., C₁-C₈ alkyl, C₁-C₆ alkyl, or C₁-C₄ alkyl). In embodiments, R⁶² is a substituted alkyl (e.g., C₁-C₈ alkyl, C₁-C₆ alkyl, or C₁-C₄ alkyl). In embodiments, R⁶² is an unsubstituted alkyl (e.g., C₁-C₈ alkyl, C₁-C₆ alkyl, or C₁-C₄ alkyl). In embodiments, R⁶² is a substituted or unsubstituted heteroalkyl (e.g., 2 to 8 membered heteroalkyl, 2 to 6 membered heteroalkyl, or 2 to 4 membered heteroalkyl). In embodiments, R⁶² is a substituted heteroalkyl (e.g., 2 to 8 membered heteroalkyl, 2 to 6 membered heteroalkyl, or 2 to 4 membered heteroalkyl). In embodiments, R⁶² is an unsubstituted heteroalkyl (e.g., 2 to 8 membered heteroalkyl, 2 to 6 membered heteroalkyl, or 2 to 4 membered heteroalkyl). In embodiments, R⁶² is a substituted or unsubstituted cycloalkyl (e.g., C₃-C₈ cycloalkyl, C₃-C₆ cycloalkyl, or C₅-C₆ cycloalkyl). In embodiments, R⁶² is a substituted cycloalkyl (e.g., C₃-C₈ cycloalkyl, C₃-C₆ cycloalkyl, or C₅-C₆ cycloalkyl). In embodiments, R⁶² is an unsubstituted cycloalkyl (e.g., C₃-C₈ cycloalkyl, C₃-C₆ cycloalkyl, or C₅-C₆ cycloalkyl). In embodiments, R⁶² is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁶² is a substituted heterocycloalkyl (e.g., 3 to 8 membered heterocycloalkyl, 3 to 6 membered heterocycloalkyl, or 5 to 6 membered heterocycloalkyl). In embodiments, R⁶² is an unsubstituted heterocycloalkyl (e.g., 3 to 8 membered heterocycloalkyl, 3 to 6 membered heterocycloalkyl, or 5 to 6 membered heterocycloalkyl). In embodiments, R⁶² is a substituted or unsubstituted aryl (e.g., C₆-C₁₀ aryl, C₁₀ aryl, or phenyl). In embodiments, R⁶² is a substituted aryl (e.g., C₆-C₁₀ aryl, C₁₀ aryl, or phenyl). In embodiments, R⁶² is an unsubstituted aryl (e.g., C₆-C₁₀ aryl, C₁₀ aryl, or phenyl). In embodiments, R⁶² is a substituted or unsubstituted heteroaryl (e.g., 5 to 10 membered heteroaryl, 5 to 9 membered heteroaryl, or 5 to 6 membered heteroaryl). In embodiments, R⁶² is a substituted heteroaryl (e.g., 5 to 10 membered heteroaryl, 5 to 9 membered heteroaryl, or 5 to 6 membered heteroaryl). In embodiments, R⁶² is an unsubstituted heteroaryl (e.g., 5 to 10 membered heteroaryl, 5 to 9 membered heteroaryl, or 5 to 6 membered heteroaryl).

In embodiments, R⁶¹ and R⁶² are together oxo.

In embodiments, R^{61A}, R^{61B}, R^{61C} and R^{61D} are independently hydrogen, halogen, —CF₃, —CCl₃, —CBr₃, —CI₃, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). R^{61B} and R^{61C} substituents bonded to the same nitrogen atom may optionally be joined to form a substituted or unsubstituted heterocycloalkyl (e.g., 3 to 8 membered heterocycloalkyl, 3 to 6 membered heterocycloalkyl, or 5 to 6 membered heterocycloalkyl) or substituted or unsubstituted heteroaryl (e.g., 5 to 10 membered heteroaryl, 5 to 9 membered heteroaryl, or 5 to 6 membered heteroaryl).

In embodiments, when R⁶¹ is attached to nitrogen, R⁶¹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₆₁H, —SOᵥ₆₁NH₂, —C(O)H, —C(O)OH, —C(O)NH₂, —OH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N(CH₃)₂, —NH(CH₃), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁶¹ is hydrogen.

R³⁷, R⁴⁰, R⁴³, R⁴⁶, R⁴⁹, R⁵², and R⁵⁵ are independently oxo, halogen, —CCl₃, —CBr₃, —CF₃, —Cl₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

R²⁹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₉H, —SOᵥ₂₉NH₂, —NHNH₂, —ONH₂, —NHC(O)NHNH₂, —NHC(O)NH₂, —N(O)ₘ₂₉, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R²⁹ is a substituted or unsubstituted alkyl (e.g., C₁-C₈ alkyl, C₁-C₆ alkyl, or C₁-C₄ alkyl). In embodiments, R²⁹ is a substituted alkyl (e.g., C₁-C₈ alkyl, C₁-C₆ alkyl, or C₁-C₄ alkyl). In embodiments, R²⁹ is an unsubstituted alkyl (e.g., C₁-C₈ alkyl, C₁-C₆ alkyl, or C₁-C₄ alkyl). In embodiments, R²⁹ is a substituted or unsubstituted heteroalkyl (e.g., 2 to 8 membered heteroalkyl, 2 to 6 membered heteroalkyl, or 2 to 4 membered heteroalkyl). In embodiments, R²⁹ is a substituted heteroalkyl (e.g., 2 to 8 membered heteroalkyl, 2 to 6 membered heteroalkyl, or 2 to 4 membered heteroalkyl). In embodiments, R²⁹ is an unsubstituted heteroalkyl (e.g., 2 to 8 membered heteroalkyl, 2 to 6 membered heteroalkyl, or 2 to 4 membered heteroalkyl). In embodiments, R²⁹ is a substituted or unsubstituted cycloalkyl (e.g., C₃-C₈ cycloalkyl, C₃-C₆ cycloalkyl, or C₅-C₆ cycloalkyl). In embodiments, R²⁹ is a substituted cycloalkyl (e.g., C₃-C₈ cycloalkyl, C₃-C₆ cycloalkyl, or C₅-C₆ cycloalkyl). In embodiments, R²⁹ is an unsubstituted cycloalkyl (e.g., C₃-C₈ cycloalkyl, C₃-C₆ cycloalkyl, or C₅-C₆ cycloalkyl). In embodiments, R²⁹ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R²⁹ is a substituted heterocycloalkyl (e.g., 3 to 8 membered heterocycloalkyl, 3 to 6 membered heterocycloalkyl, or 5 to 6 membered heterocycloalkyl). In embodiments, R²⁹ is an unsubstituted heterocycloalkyl (e.g., 3 to 8 membered heterocycloalkyl, 3 to 6 membered heterocycloalkyl, or 5 to 6 membered heterocycloalkyl). In embodiments, R²⁹ is a substituted or unsubstituted aryl (e.g., C₆-C₁₀ aryl, C₁₀ aryl, or phenyl). In embodiments, R²⁹ is a substituted aryl (e.g., C₆-C₁₀ aryl, C₁₀ aryl, or phenyl). In embodiments, R²⁹ is an unsubstituted aryl (e.g., C₆-C₁₀ aryl, C₁₀ aryl, or phenyl). In embodiments, R²⁹ is a substituted or unsubstituted heteroaryl (e.g., 5 to 10 membered heteroaryl, 5 to 9 membered heteroaryl, or 5 to 6 membered heteroaryl). In embodiments, R²⁹ is a substituted heteroaryl (e.g., 5 to 10 membered heteroaryl, 5 to 9 membered heteroaryl, or 5 to 6 membered heteroaryl). In embodiments, R²⁹ is an unsubstituted heteroaryl (e.g., 5 to 10 membered heteroaryl, 5 to 9 membered heteroaryl, or 5 to 6 membered heteroaryl).

In embodiments, R²⁹ is hydrogen, halogen, substituted or unsubstituted alkyl or substituted or unsubstituted heteroalkyl. In embodiments, R²⁹ is hydrogen or halogen. In embodiments, R²⁹ is hydrogen.

R³⁰ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃₀H, —SOᵥ₃₀NH₂, —NHNH₂, —ONH₂, —NHC(O)NHNH₂, —NHC(O)NH₂, —N(O)ₘ₃₀₀, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R³⁰ is a substituted or unsubstituted alkyl (e.g., C₁-C₈ alkyl, C₁-C₆ alkyl, or C₁-C₄ alkyl). In embodiments, R³⁰ is a substituted alkyl (e.g., C₁-C₈ alkyl, C₁-C₆ alkyl, or C₁-C₄ alkyl). In embodiments, R³⁰ is an unsubstituted alkyl (e.g., C₁-C₈ alkyl, C₁-C₆ alkyl, or C₁-C₄ alkyl). In embodiments, R³⁰ is a substituted or unsubstituted heteroalkyl (e.g., 2 to 8 membered heteroalkyl, 2 to 6 membered heteroalkyl, or 2 to 4 membered heteroalkyl). In embodiments, R³⁰ is a substituted heteroalkyl (e.g., 2 to 8 membered heteroalkyl, 2 to 6 membered heteroalkyl, or 2 to 4 membered heteroalkyl). In embodiments, R³⁰ is an unsubstituted heteroalkyl (e.g., 2 to 8 membered heteroalkyl, 2 to 6 membered heteroalkyl, or 2 to 4 membered heteroalkyl). In embodiments, R³⁰ is a substituted or unsubstituted cycloalkyl (e.g., C₃-C₈ cycloalkyl, C₃-C₆ cycloalkyl, or C₅-C₆ cycloalkyl). In embodiments, R³⁰ is a substituted cycloalkyl (e.g., C₃-C₈ cycloalkyl, C₃-C₆ cycloalkyl, or C₅-C₆ cycloalkyl). In embodiments, R³⁰ is an unsubstituted cycloalkyl (e.g., C₃-C₈ cycloalkyl, C₃-C₆ cycloalkyl, or C₅-C₆ cycloalkyl). In embodiments, R³⁰ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³⁰ is a substituted heterocycloalkyl (e.g., 3 to 8 membered heterocycloalkyl, 3 to 6 membered heterocycloalkyl, or 5 to 6 membered heterocycloalkyl). In embodiments, R³⁰ is an unsubstituted heterocycloalkyl (e.g., 3 to 8 membered heterocycloalkyl, 3 to 6 membered heterocycloalkyl, or 5 to 6 membered heterocycloalkyl). In embodiments, R³⁰ is a substituted or unsubstituted aryl (e.g., C₆-C₁₀ aryl, C₁₀ aryl, or phenyl). In embodiments, R³⁰ is a substituted aryl (e.g., C₆-C₁₀ aryl, C₁₀ aryl, or phenyl). In embodiments, R³⁰ is an unsubstituted aryl (e.g., C₆-C₁₀ aryl, C₁₀ aryl, or phenyl). In embodiments, R³⁰ is a substituted or unsubstituted heteroaryl (e.g., 5 to 10 membered heteroaryl, 5 to 9 membered heteroaryl, or 5 to 6 membered heteroaryl). In embodiments, R³⁰ is a substituted heteroaryl (e.g., 5 to 10 membered heteroaryl, 5 to 9 membered heteroaryl, or 5 to 6 membered heteroaryl). In embodiments, R³⁰ is an unsubstituted heteroaryl (e.g., 5 to 10 membered heteroaryl, 5 to 9 membered heteroaryl, or 5 to 6 membered heteroaryl).

In embodiments, R³⁰ is hydrogen, halogen, substituted or unsubstituted alkyl or substituted or unsubstituted heteroalkyl. In embodiments, R³⁰ is hydrogen or halogen. In embodiments, R³⁰ is hydrogen.

R³¹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃₁H, —SOᵥ₃₁NH₂, —NHNH₂, —ONH₂, —NHC(O)NHNH₂, —NHC(O)NH₂, —N(O)ₘ₃₁, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R³¹ is a substituted or unsubstituted alkyl (e.g., C₁-C₈ alkyl, C₁-C₆ alkyl, or C₁-C₄ alkyl). In embodiments, R³¹ is a substituted alkyl (e.g., C₁-C₈ alkyl, C₁-C₆ alkyl, or C₁-C₄ alkyl). In embodiments, R³¹ is an unsubstituted alkyl (e.g., C₁-C₈ alkyl, C₁-C₆ alkyl, or C₁-C₄ alkyl). In embodiments, R³¹ is a substituted or unsubstituted heteroalkyl (e.g., 2 to 8 membered heteroalkyl, 2 to 6 membered heteroalkyl, or 2 to 4 membered heteroalkyl). In embodiments, R³¹ is a substituted heteroalkyl (e.g., 2 to 8 membered heteroalkyl, 2 to 6 membered heteroalkyl, or 2 to 4 membered heteroalkyl). In embodiments, R³¹ is an unsubstituted heteroalkyl (e.g., 2 to 8 membered heteroalkyl, 2 to 6 membered heteroalkyl, or 2 to 4 membered heteroalkyl). In embodiments, R³¹ is a substituted or unsubstituted cycloalkyl (e.g., C₃-C₈ cycloalkyl, C₃-C₆ cycloalkyl, or C₅-C₆ cycloalkyl). In embodiments, R³¹ is a substituted cycloalkyl (e.g., C₃-C₈ cycloalkyl, C₃-C₆ cycloalkyl, or C₅-C₆ cycloalkyl). In embodiments, R³¹ is an unsubstituted cycloalkyl (e.g., C₃-C₈ cycloalkyl, C₃-C₆ cycloalkyl, or C₅-C₆ cycloalkyl). In embodiments, R³¹ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³¹ is a substituted heterocycloalkyl (e.g., 3 to 8 membered heterocycloalkyl, 3 to 6 membered heterocycloalkyl, or 5 to 6 membered heterocycloalkyl). In embodiments, R³¹ is an unsubstituted heterocycloalkyl (e.g., 3 to 8 membered heterocycloalkyl, 3 to 6 membered heterocycloalkyl, or 5 to 6 membered heterocycloalkyl). In embodiments, R³¹ is a substituted or unsubstituted aryl (e.g., C₆-C₁₀ aryl, C₁₀ aryl, or phenyl). In embodiments, R³¹ is a substituted aryl (e.g., C₆-C₁₀ aryl, C₁₀ aryl, or phenyl). In embodiments, R³¹ is an unsubstituted aryl (e.g., C₆-C₁₀ aryl, C₁₀ aryl, or phenyl). In embodiments, R³¹ is a substituted or unsubstituted heteroaryl (e.g., 5 to 10 membered heteroaryl, 5 to 9 membered heteroaryl, or 5 to 6 membered heteroaryl). In embodiments, R³¹ is a substituted heteroaryl (e.g., 5 to 10 membered heteroaryl, 5 to 9 membered heteroaryl, or 5 to 6 membered heteroaryl). In embodiments, R³¹ is an unsubstituted heteroaryl (e.g., 5 to 10 membered heteroaryl, 5 to 9 membered heteroaryl, or 5 to 6 membered heteroaryl).

In embodiments, R³¹ is hydrogen, halogen, substituted or unsubstituted alkyl or substituted or unsubstituted heteroalkyl. In embodiments, R³¹ is hydrogen or halogen. In embodiments, R³¹ is hydrogen.

R³² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃₂H, —SOᵥ₃₂NH₂, —NHNH₂, —ONH₂, —NHC(O)NHNH₂, —NHC(O)NH₂, —N(O)ₘ₃₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R³² is a substituted or unsubstituted alkyl (e.g., C₁-C₈ alkyl, C₁-C₆ alkyl, or C₁-C₄ alkyl). In embodiments, R³² is a substituted alkyl (e.g., C₁-C₈ alkyl, C₁-C₆ alkyl, or C₁-C₄ alkyl). In embodiments, R³² is an unsubstituted alkyl (e.g., C₁-C₈ alkyl, C₁-C₆ alkyl, or C₁-C₄ alkyl). In embodiments, R³² is a substituted or unsubstituted heteroalkyl (e.g., 2 to 8 membered heteroalkyl, 2 to 6 membered heteroalkyl, or 2 to 4 membered heteroalkyl). In embodiments, R³² is a substituted heteroalkyl (e.g., 2 to 8 membered heteroalkyl, 2 to 6 membered heteroalkyl, or 2 to 4 membered heteroalkyl). In embodiments, R³² is an unsubstituted heteroalkyl (e.g., 2 to 8 membered heteroalkyl, 2 to 6 membered heteroalkyl, or 2 to 4 membered heteroalkyl). In embodiments, R³² is a substituted or unsubstituted cycloalkyl (e.g., C₃-C₈ cycloalkyl, C₃-C₆ cycloalkyl, or C₅-C₆ cycloalkyl). In embodiments, R³² is a substituted cycloalkyl (e.g., C₃-C₈ cycloalkyl, C₃-C₆ cycloalkyl, or C₅-C₆ cycloalkyl). In embodiments, R³² is an unsubstituted cycloalkyl (e.g., C₃-C₈ cycloalkyl, C₃-C₆ cycloalkyl, or C₅-C₆ cycloalkyl). In embodiments, R³² is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³² is a substituted heterocycloalkyl (e.g., 3 to 8 membered heterocycloalkyl, 3 to 6 membered heterocycloalkyl, or 5 to 6 membered heterocycloalkyl). In embodiments, R³² is an unsubstituted heterocycloalkyl (e.g., 3 to 8 membered heterocycloalkyl, 3 to 6 membered heterocycloalkyl, or 5 to 6 membered heterocycloalkyl). In embodiments, R³² is a substituted or unsubstituted aryl (e.g., C₆-C₁₀ aryl, C₁₀ aryl, or phenyl). In embodiments, R³² is a substituted aryl (e.g., C₆-C₁₀ aryl, C₁₀ aryl, or phenyl). In embodiments, R³² is an unsubstituted aryl (e.g., C₆-C₁₀ aryl, C₁₀ aryl, or phenyl). In embodiments, R³² is a substituted or unsubstituted heteroaryl (e.g., 5 to 10 membered heteroaryl, 5 to 9 membered heteroaryl, or 5 to 6 membered heteroaryl). In embodiments, R³² is a substituted heteroaryl (e.g., 5 to 10 membered heteroaryl, 5 to 9 membered heteroaryl, or 5 to 6 membered heteroaryl). In embodiments, R³² is an unsubstituted heteroaryl (e.g., 5 to 10 membered heteroaryl, 5 to 9 membered heteroaryl, or 5 to 6 membered heteroaryl).

In embodiments, R³² is hydrogen, halogen, substituted or unsubstituted alkyl or substituted or unsubstituted heteroalkyl. In embodiments, R³² is hydrogen or halogen. In embodiments, R³² is hydrogen.

R³⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃₄H, —SOᵥ₃₄NH₂, —NHNH₂, —ONH₂, —NHC(O)NHNH₂, —NHC(O)NH₂, —N(O)ₘ₃₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R³⁴ is a substituted or unsubstituted alkyl (e.g., C₁-C₈ alkyl, C₁-C₆ alkyl, or C₁-C₄ alkyl). In embodiments, R³⁴ is a substituted alkyl (e.g., C₁-C₈ alkyl, C₁-C₆ alkyl, or C₁-C₄ alkyl). In embodiments, R³⁴ is an unsubstituted alkyl (e.g., C₁-C₈ alkyl, C₁-C₆ alkyl, or C₁-C₄ alkyl). In embodiments, R³⁴ is a substituted or unsubstituted heteroalkyl (e.g., 2 to 8 membered heteroalkyl, 2 to 6 membered heteroalkyl, or 2 to 4 membered heteroalkyl). In embodiments, R³⁴ is a substituted heteroalkyl (e.g., 2 to 8 membered heteroalkyl, 2 to 6 membered heteroalkyl, or 2 to 4 membered heteroalkyl). In embodiments, R³⁴ is an unsubstituted heteroalkyl (e.g., 2 to 8 membered heteroalkyl, 2 to 6 membered heteroalkyl, or 2 to 4 membered heteroalkyl). In embodiments, R³⁴ is a substituted or unsubstituted cycloalkyl (e.g., C₃-C₈ cycloalkyl, C₃-C₆ cycloalkyl, or C₅-C₆ cycloalkyl). In embodiments, R³⁴ is a substituted cycloalkyl (e.g., C₃-C₈ cycloalkyl, C₃-C₆ cycloalkyl, or C₅-C₆ cycloalkyl). In embodiments, R³⁴ is an unsubstituted cycloalkyl (e.g., C₃-C₈ cycloalkyl, C₃-C₆ cycloalkyl, or C₅-C₆ cycloalkyl). In embodiments, R³⁴ is a substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³⁴ is a substituted heterocycloalkyl (e.g., 3 to 8 membered heterocycloalkyl, 3 to 6 membered heterocycloalkyl, or 5 to 6 membered heterocycloalkyl). In embodiments, R³⁴ is an unsubstituted heterocycloalkyl (e.g., 3 to 8 membered heterocycloalkyl, 3 to 6 membered heterocycloalkyl, or 5 to 6 membered heterocycloalkyl). In embodiments, R³⁴ is a substituted or unsubstituted aryl (e.g., C₆-C₁₀ aryl, C₁₀ aryl, or phenyl). In embodiments, R³⁴ is a substituted aryl (e.g., C₆-C₁₀ aryl, C₁₀ aryl, or phenyl). In embodiments, R³⁴ is an unsubstituted aryl (e.g., C₆-C₁₀ aryl, C₁₀ aryl, or phenyl). In embodiments, R³⁴ is a substituted or unsubstituted heteroaryl (e.g., 5 to 10 membered heteroaryl, 5 to 9 membered heteroaryl, or 5 to 6 membered heteroaryl). In embodiments, R³⁴ is a substituted heteroaryl (e.g., 5 to 10 membered heteroaryl, 5 to 9 membered heteroaryl, or 5 to 6 membered heteroaryl). In embodiments, R³⁴ is an unsubstituted heteroaryl (e.g., 5 to 10 membered heteroaryl, 5 to 9 membered heteroaryl, or 5 to 6 membered heteroaryl).

In embodiments, R³⁴ is hydrogen, halogen, substituted or unsubstituted alkyl or substituted or unsubstituted heteroalkyl. In embodiments, R³⁴ is hydrogen or halogen. In embodiments, R³⁴ is hydrogen.

In embodiments, X¹⁰ is —O—, —S— or N(R³⁴). In embodiments, X¹⁰ is —O—. In embodiments, X¹⁰ is —S—. In embodiments, X¹⁰ is —N(R³⁴)—. In embodiments, X¹⁰ is —N(H)—.

In embodiments, z1 is 0. In embodiments, z1 is 1. In embodiments, z1 is 2. In embodiments, z1 is 3. In embodiments, z1 is 4.

In embodiments, z2 is 0. In embodiments, z2 is 1. In embodiments, z2 is 2. In embodiments, z2 is 3. In embodiments, z2 is 4. In embodiments, z2 is 5. In embodiments, z2 is 6. In embodiments, z2 is 7. In embodiments, z2 is 8.

In embodiments, z3 is 0. In embodiments, z3 is 1. In embodiments, z3 is 2. In embodiments, z3 is 3. In embodiments, z3 is 4.

In embodiments, z4 is 0. In embodiments, z4 is 1. In embodiments, z4 is 2. In embodiments, z4 is 3. In embodiments, z4 is 4.

In embodiments, z5 is 0. In embodiments, z5 is 1. In embodiments, z5 is 2. In embodiments, z5 is 3. In embodiments, z5 is 4. In embodiments, z5 is 5. In embodiments, z5 is 6. In embodiments, z5 is 7. In embodiments, z5 is 8.

In embodiments, z6 is 0. In embodiments, z6 is 1.

In embodiments, z9 is 0. In embodiments, z9 is 1. In embodiments, z9 is 2. In embodiments, z9 is 3. In embodiments, z9 is 4. In embodiments, z9 is 5. In embodiments, z9 is 6.

In embodiments p1 is 1. In embodiments, p1 is 2.

In embodiments, n1 is 0. In embodiments, n1 is 1. In embodiments, n1 is 2. In embodiments, n1 is 3. In embodiments, n1 is 4.

In embodiments, n1.1 is 0. In embodiments, n1.1 is 1. In embodiments, n1.1 is 2. In embodiments, n1.1 is 3. In embodiments, n1.1 is 4.

In embodiments, n1.2 is 0. In embodiments, n1.2 is 1. In embodiments, n1.2 is 2. In embodiments, n1.2 is 3. In embodiments, n1.2 is 4.

In embodiments, n1.3 is 0. In embodiments, n1.3 is 1. In embodiments, n1.3 is 2. In embodiments, n1.3 is 3. In embodiments, n1.3 is 4.

In embodiments, n1.4 is 0. In embodiments, n1.4 is 1. In embodiments, n1.4 is 2. In embodiments, n1.4 is 3. In embodiments, n1.4 is 4.

In embodiments, n2 is 0. In embodiments, n2 is 1. In embodiments, n2 is 2. In embodiments, n2 is 3. In embodiments, n2 is 4.

In embodiments, n3 is 0. In embodiments, n3 is 1. In embodiments, n3 is 2. In embodiments, n3 is 3. In embodiments, n3 is 4.

In embodiments, n4 is 0. In embodiments, n4 is 1. In embodiments, n4 is 2. In embodiments, n4 is 3. In embodiments, n4 is 4.

In embodiments, n5 is 0. In embodiments, n5 is 1. In embodiments, n5 is 2. In embodiments, n5 is 3. In embodiments, n5 is 4.

In embodiments, n6 is 0. In embodiments, n6 is 1. In embodiments, n6 is 2. In embodiments, n6 is 3. In embodiments, n6 is 4.

In embodiments, n7 is 0. In embodiments, n7 is 1. In embodiments, n7 is 2. In embodiments, n7 is 3. In embodiments, n7 is 4.

In embodiments, n8 is 0. In embodiments, n8 is 1. In embodiments, n8 is 2. In embodiments, n8 is 3. In embodiments, n8 is 4.

In embodiments, n9 is 0. In embodiments, n9 is 1. In embodiments, n9 is 2. In embodiments, n9 is 3. In embodiments, n9 is 4.

In embodiments, n10 is 0. In embodiments, n10 is 1. In embodiments, n10 is 2. In embodiments, n10 is 3. In embodiments, n10 is 4.

In embodiments, n12 is 0. In embodiments, n12 is 1. In embodiments, n12 is 2. In embodiments, n12 is 3. In embodiments, n12 is 4.

In embodiments, n13 is 0. In embodiments, n13 is 1. In embodiments, n13 is 2. In embodiments, n13 is 3. In embodiments, n13 is 4.

In embodiments, n14 is 0. In embodiments, n14 is 1. In embodiments, n14 is 2. In embodiments, n14 is 3. In embodiments, n14 is 4.

In embodiments, n15 is 0. In embodiments, n15 is 1. In embodiments, n15 is 2. In embodiments, n15 is 3. In embodiments, n15 is 4.

In embodiments, n16 is 0. In embodiments, n16 is 1. In embodiments, n16 is 2. In embodiments, n16 is 3. In embodiments, n16 is 4.

In embodiments, n17 is 0. In embodiments, n17 is 1. In embodiments, n17 is 2. In embodiments, n17 is 3. In embodiments, n17 is 4.

In embodiments, n18 is 0. In embodiments, n18 is 1. In embodiments, n18 is 2. In embodiments, n18 is 3. In embodiments, n18 is 4.

In embodiments, n19 is 0. In embodiments, n19 is 1. In embodiments, n19 is 2. In embodiments, n19 is 3. In embodiments, n19 is 4.

In embodiments, n20 is 0. In embodiments, n20 is 1. In embodiments, n20 is 2. In embodiments, n20 is 3. In embodiments, n20 is 4.

In embodiments, n21 is 0. In embodiments, n21 is 1. In embodiments, n21 is 2. In embodiments, n21 is 3. In embodiments, n21 is 4.

In embodiments, n22 is 0. In embodiments, n22 is 1. In embodiments, n22 is 2. In embodiments, n22 is 3. In embodiments, n22 is 4.

In embodiments, n23 is 0. In embodiments, n23 is 1. In embodiments, n23 is 2. In embodiments, n23 is 3. In embodiments, n23 is 4.

In embodiments, n24 is 0. In embodiments, n24 is 1. In embodiments, n24 is 2. In embodiments, n24 is 3. In embodiments, n24 is 4.

In embodiments, n25 is 0. In embodiments, n25 is 1. In embodiments, n25 is 2. In embodiments, n25 is 3. In embodiments, n25 is 4.

In embodiments, n26 is 0. In embodiments, n26 is 1. In embodiments, n26 is 2. In embodiments, n26 is 3. In embodiments, n26 is 4.

In embodiments, n27 is 0. In embodiments, n27 is 1. In embodiments, n27 is 2. In embodiments, n27 is 3. In embodiments, n27 is 4.

In embodiments, n28 is 0. In embodiments, n28 is 1. In embodiments, n28 is 2. In embodiments, n28 is 3. In embodiments, n28 is 4.

In embodiments, n29 is 0. In embodiments, n29 is 1. In embodiments, n29 is 2. In embodiments, n29 is 3. In embodiments, n29 is 4.

In embodiments, n30 is 0. In embodiments, n30 is 1. In embodiments, n30 is 2. In embodiments, n30 is 3. In embodiments, n30 is 4.

In embodiments, n31 is 0. In embodiments, n31 is 1. In embodiments, n31 is 2. In embodiments, n31 is 3. In embodiments, n31 is 4.

In embodiments, n32 is 0. In embodiments, n32 is 1. In embodiments, n32 is 2. In embodiments, n32 is 3. In embodiments, n32 is 4.

In embodiments, n33 is 0. In embodiments, n33 is 1. In embodiments, n33 is 2. In embodiments, n33 is 3. In embodiments, n33 is 4.

In embodiments, n34 is 0. In embodiments, n34 is 1. In embodiments, n34 is 2. In embodiments, n34 is 3. In embodiments, n34 is 4.

In embodiments, n59 is 0. In embodiments, n59 is 1. In embodiments, n59 is 2. In embodiments, n59 is 3. In embodiments, n59 is 4.

In embodiments, n60 is 0. In embodiments, n60 is 1. In embodiments, n60 is 2. In embodiments, n60 is 3. In embodiments, n60 is 4.

In embodiments, n61 is 0. In embodiments, n61 is 1. In embodiments, n61 is 2. In embodiments, n61 is 3. In embodiments, n61 is 4.

In embodiments, n62 is 0. In embodiments, n62 is 1. In embodiments, n62 is 2. In embodiments, n62 is 3. In embodiments, n62 is 4.

In embodiments, m1 is 1. In embodiments, m1 is 2.

In embodiments, m1.1 is 1. In embodiments, m1.1 is 2.

In embodiments, m1.2 is 1. In embodiments, m1.2 is 2.

In embodiments, m1.3 is 1. In embodiments, m1.3 is 2.

In embodiments, m1.4 is 1. In embodiments, m1.4 is 2.

In embodiments, m2 is 1. In embodiments, m2 is 2.

In embodiments, m3 is 1. In embodiments, m3 is 2.

In embodiments, m4 is 1. In embodiments, m4 is 2.

In embodiments, m5 is 1. In embodiments, m5 is 2.

In embodiments, m6 is 1. In embodiments, m6 is 2.

In embodiments, m7 is 1. In embodiments, m7 is 2.

In embodiments, m8 is 1. In embodiments, m8 is 2.

In embodiments, m9 is 1. In embodiments, m9 is 2.

In embodiments, m10 is 1. In embodiments, m10 is 2.

In embodiments, m12 is 1. In embodiments, m12 is 2.

In embodiments, m13 is 1. In embodiments, m13 is 2.

In embodiments, m14 is 1. In embodiments, m14 is 2.

In embodiments, m15 is 1. In embodiments, m15 is 2.

In embodiments, m16 is 1. In embodiments, m16 is 2.

In embodiments, m17 is 1. In embodiments, m17 is 2.

In embodiments, m18 is 1. In embodiments, m18 is 2.

In embodiments, m19 is 1. In embodiments, m19 is 2.

In embodiments, m20 is 1. In embodiments, m20 is 2.

In embodiments, m21 is 1. In embodiments, m21 is 2.

In embodiments, m22 is 1. In embodiments, m22 is 2.

In embodiments, m23 is 1. In embodiments, m23 is 2.

In embodiments, m24 is 1. In embodiments, m24 is 2.

In embodiments, m25 is 1. In embodiments, m25 is 2.

In embodiments, m26 is 1. In embodiments, m26 is 2.

In embodiments, m27 is 1. In embodiments, m27 is 2.

In embodiments, m28 is 1. In embodiments, m28 is 2.

In embodiments, m29 is 1. In embodiments, m29 is 2.

In embodiments, m30 is 1. In embodiments, m30 is 2.

In embodiments, m31 is 1. In embodiments, m32 is 2.

In embodiments, m32 is 1. In embodiments, m32 is 2.

In embodiments, m33 is 1. In embodiments, m33 is 2.

In embodiments, m34 is 1. In embodiments, m34 is 2.

In embodiments, m59 is 1. In embodiments, m59 is 2.

In embodiments, m60 is 1. In embodiments, m60 is 2.

In embodiments, m61 is 1. In embodiments, m61 is 2.

In embodiments, m62 is 1. In embodiments, m62 is 2.

In embodiments, v1 is 1. In embodiments, v1 is 2.

In embodiments, v1.1 is 1. In embodiments, v1.1 is 2.

In embodiments, v1.2 is 1. In embodiments, v1.2 is 2.

In embodiments, v1.3 is 1. In embodiments, v1.3 is 2.

In embodiments, v1.4 is 1. In embodiments, v1.4 is 2.

In embodiments, v2 is 1. In embodiments, v2 is 2.

In embodiments, v3 is 1. In embodiments, v3 is 2.

In embodiments, v4 is 1. In embodiments, v4 is 2.

In embodiments, v5 is 1. In embodiments, v5 is 2.

In embodiments, v6 is 1. In embodiments, v6 is 2.

In embodiments, v7 is 1. In embodiments, v7 is 2.

In embodiments, v8 is 1. In embodiments, v8 is 2.

In embodiments, v9 is 1. In embodiments, v9 is 2.

In embodiments, v10 is 1. In embodiments, v10 is 2.

In embodiments, v12 is 1. In embodiments, v12 is 2.

In embodiments, v13 is 1. In embodiments, v13 is 2.

In embodiments, v14 is 1. In embodiments, v14 is 2.

In embodiments, v15 is 1. In embodiments, v15 is 2.

In embodiments, v16 is 1. In embodiments, v16 is 2.

In embodiments, v17 is 1. In embodiments, v17 is 2.

In embodiments, v18 is 1. In embodiments, v18 is 2.

In embodiments, v19 is 1. In embodiments, v19 is 2.

In embodiments, v20 is 1. In embodiments, v20 is 2.

In embodiments, v21 is 1. In embodiments, v21 is 2.

In embodiments, v22 is 1. In embodiments, v22 is 2.

In embodiments, v23 is 1. In embodiments, v23 is 2.

In embodiments, v24 is 1. In embodiments, v24 is 2.

In embodiments, v25 is 1. In embodiments, v25 is 2.

In embodiments, v26 is 1. In embodiments, v26 is 2.

In embodiments, v27 is 1. In embodiments, v27 is 2.

In embodiments, v28 is 1. In embodiments, v28 is 2.

In embodiments, v29 is 1. In embodiments, v29 is 2.

In embodiments, v30 is 1. In embodiments, v30 is 2.

In embodiments, v31 is 1. In embodiments, v31 is 2.

In embodiments, v32 is 1. In embodiments, v32 is 2.

In embodiments, v33 is 1. In embodiments, v33 is 2.

In embodiments, v34 is 1. In embodiments, v34 is 2.

In embodiments, v59 is 1. In embodiments, v59 is 2.

In embodiments, v60 is 1. In embodiments, v60 is 2.

In embodiments, v61 is 1. In embodiments, v61 is 2.

In embodiments, v62 is 1. In embodiments, v62 is 2.

In embodiments, X¹ is —C(R⁸)—. In embodiments, X¹ is —N—. In embodiments, X¹ is

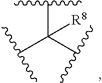

wherein R⁸ is as described herein, including embodiments. In embodiments, X¹ is

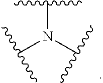 [SMILES: *N(*)*]

In embodiments, X² is —C(R⁹)(R¹⁰)—. In embodiments, X² is —N(R⁹)—. In embodiments, X² is —N(H)—. In embodiments, X² is

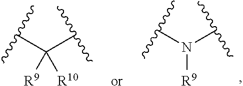

wherein R⁹ and R¹⁰ are as described herein, including embodiments. In embodiments, X² is

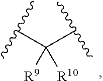

wherein R⁹ and R¹⁰ are as described herein, including embodiments. In embodiments, X² is

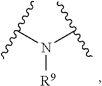

wherein R⁹ is as described herein, including embodiments

In embodiments, X³ is —C(R¹²)(R¹³)—. In embodiments, X³ is N(R¹²)—. In embodiments, X³ is

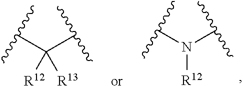

wherein R¹² and R¹³ are as described herein, including embodiments.

In embodiments, X⁴ is —C(R¹⁴)(R¹⁵)—. In embodiments, X⁴ is —N(R¹⁴)—. In embodiments, X⁴ is

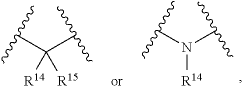

wherein R¹⁴ and R¹⁵ are as described herein, including embodiments. In embodiments, X³ is

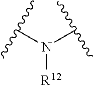

### and X⁴ is

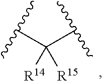

wherein R¹², R¹⁴ and R¹⁵ are as described herein, including embodiments.

In embodiments, X³ is —N(R¹²)— and X⁴ is —C(R¹⁴)(R¹⁵)—. In embodiments, X³ is —C(R¹²)(R¹³)— and X⁴ is —N(R¹⁴)—. In embodiments, X³ is

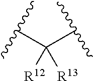

### and X⁴ is

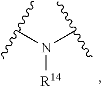

wherein R¹², R¹³ and R¹⁴ are as described herein, including embodiments.

In embodiments, X⁵ is —O—. In embodiments, X⁵ is —N(R¹⁶)—. In embodiments, X⁵ is

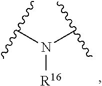

wherein R¹⁶ is as described herein, including embodiments.

In embodiments, X⁶ is —C(R¹⁸)(R¹⁹)—. In embodiments, X⁶ is —N(R¹⁸)—. In embodiments, X⁶ is

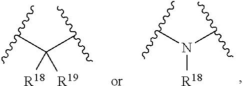

wherein R¹⁸ and R¹⁹ are as described herein, including embodiments. In embodiments, X⁶ is

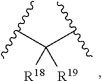

wherein R¹⁸ and R¹⁹ are as described herein, including embodiments. In embodiments, X⁶ is

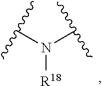

wherein R is as described herein, including embodiments.

In embodiments, X⁷ is —C(R²⁰)(R²¹)—. In embodiments, X⁷ is —N(R²⁰)—. In embodiments, X⁷ is

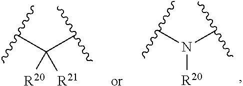

wherein R²⁰ and R²¹ are as described herein, including embodiments. In embodiments, X⁷ is

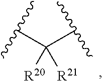

wherein R²⁰ and R²¹ are as described herein, including embodiments. In embodiments, X⁷ is

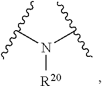

wherein R²⁰ is as described herein, including embodiments.

In embodiments, X⁶ is —N(R¹⁸), X⁷ is —C(R²⁰)(R²¹), and R²⁰ and R²¹ are hydrogen. In embodiments, X⁶ is

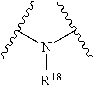

### and X⁷ is

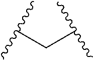 [SMILES: *C*]

(i.e. —CH₂—), wherein R¹⁸ is as described herein, including embodiments.

In embodiments, X⁸ is —O—. In embodiments, X⁸ is —N(R²²)—. In embodiments, X⁸ is

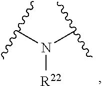

wherein R²² is as described herein, including embodiments.

In embodiments, X⁹ is —O— or —S—. In embodiments, X⁹ is —O—. In embodiments, X⁹ is —S—. In embodiments, X⁹ is —N(R³³)—. In embodiments, X⁹ is —N(H)—.

In embodiments, X¹¹ is —C(R⁶¹)(R⁶²)—. In embodiments, X¹¹ is —N(R⁶¹)—. In embodiments, X¹¹ is

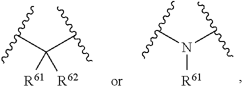

wherein R⁶¹ and R⁶² are as described herein, including embodiments.

In embodiments z3 is 0. In embodiments, z4 is 0. In embodiments, z5 is 0.

In embodiments, p1 is 1. In embodiments, p1 is 2.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl.

In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently unsubstituted alkyl, unsubstituted heteroalkyl, unsubstituted cycloalkyl, unsubstituted heterocycloalkyl, unsubstituted aryl, or unsubstituted heteroaryl.

In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted (e.g., substituted with a substituent group, a size-limited substituent group, or lower substituent group) or unsubstituted alkyl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted (e.g., substituted with a substituent group, a size-limited substituent group, or lower substituent group) alkyl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently unsubstituted alkyl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted or unsubstituted alkyl (e.g., C₁-C₈, C₁-C₆, C₁-C₄, or C₁-C₂). In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted alkyl (e.g., C₁-C₈, C₁-C₆, C₁-C₄, or C₁-C₂). In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently unsubstituted alkyl (e.g., C₁-C₈, C₁-C₆, C₁-C₄, or C₁-C₂).

In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted (e.g., substituted with a substituent group, a size-limited substituent group, or lower substituent group) or unsubstituted heteroalkyl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted (e.g., substituted with a substituent group, a size-limited substituent group, or lower substituent group) heteroalkyl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently unsubstituted heteroalkyl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted or unsubstituted heteroalkyl (e.g., 2 to 8 membered, 2 to 6 membered, 4 to 6 membered, 2 to 3 membered, or 4 to 5 membered). In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted heteroalkyl (e.g., 2 to 8 membered, 2 to 6 membered, 4 to 6 membered, 2 to 3 membered, or 4 to 5 membered). In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently an unsubstituted heteroalkyl (e.g., 2 to 8 membered, 2 to 6 membered, 4 to 6 membered, 2 to 3 membered, or 4 to 5 membered).

In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted (e.g., substituted with a substituent group, a size-limited substituent group, or lower substituent group) or unsubstituted cycloalkyl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted (e.g., substituted with a substituent group, a size-limited substituent group, or lower substituent group) cycloalkyl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently an unsubstituted cycloalkyl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted or unsubstituted cycloalkyl (e.g., C₃-C₈, C₃-C₆, C₄-C₆, Or C₅-C₆). In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted cycloalkyl (e.g., C₃-C₈, C₃-C₆, C₄-C₆, or C₅-C₆). In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently unsubstituted cycloalkyl (e.g., C₃-C₈, C₃-C₆, C₄-C₆, or C₅-C₆).

In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted (e.g., substituted with a substituent group, a size-limited substituent group, or lower substituent group) or unsubstituted heterocycloalkyl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted (e.g., substituted with a substituent group, a size-limited substituent group, or lower substituent group) heterocycloalkyl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently an unsubstituted heterocycloalkyl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted or unsubstituted heterocycloalkyl (e.g., 3 to 8 membered, 3 to 6 membered, 4 to 6 membered, 4 to 5 membered, or 5 to 6 membered). In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted heterocycloalkyl (e.g., 3 to 8 membered, 3 to 6 membered, 4 to 6 membered, 4 to 5 membered, or 5 to 6 membered). In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently an unsubstituted heterocycloalkyl (e.g., 3 to 8 membered, 3 to 6 membered, 4 to 6 membered, 4 to 5 membered, or 5 to 6 membered).

In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted (e.g., substituted with a substituent group, a size-limited substituent group, or lower substituent group) or unsubstituted aryl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted (e.g., substituted with a substituent group, a size-limited substituent group, or lower substituent group) aryl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently an unsubstituted aryl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted or unsubstituted aryl (e.g., C₆-C₁₀ or phenyl). In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted aryl (e.g., C₆-C₁₀ or phenyl). In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently an unsubstituted aryl (e.g., C₆-C₁₀ or phenyl).

In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted (e.g., substituted with a substituent group, a size-limited substituent group, or lower substituent group) or unsubstituted heteroaryl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted (e.g., substituted with a substituent group, a size-limited substituent group, or lower substituent group) heteroaryl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently an unsubstituted heteroaryl. In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted or unsubstituted heteroaryl (e.g., 5 to 10 membered, 5 to 9 membered, or 5 to 6 membered). In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently substituted heteroaryl (e.g., 5 to 10 membered, 5 to 9 membered, or 5 to 6 membered). In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently an unsubstituted heteroaryl (e.g., 5 to 10 membered, 5 to 9 membered, or 5 to 6 membered).

In embodiments, R², R³, R⁴, R⁶, R⁷, R⁸, R¹², R¹³, R¹⁴, R¹⁵, R¹⁶, R¹⁷, R¹⁸, R¹⁹, R²⁰, R²¹, R²², R²³, R²⁴, R²⁸, R²⁹, R³⁰, R³¹, R³², R³³, R³⁴, R⁵⁹, R⁶⁰, R⁶¹, and R⁶² are independently hydrogen.

In embodiments, the compound is

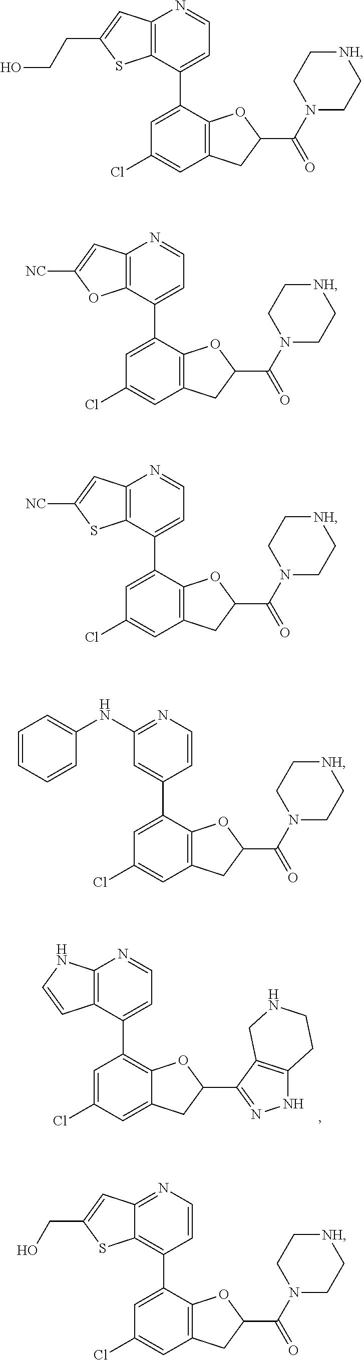 [SMILES: O=C(C1Cc2cc(Cl)cc(-c3ccnc4cc(CCO)sc34)c2O1)N1CCNCC1 ; N#Cc1cc2nccc(-c3cc(Cl)cc4c3OC(C(=O)N3CCNCC3)C4)c2o1 ; N#Cc1cc2nccc(-c3cc(Cl)cc4c3OC(C(=O)N3CCNCC3)C4)c2s1 ; O=C(C1Cc2cc(Cl)cc(-c3ccnc(Nc4ccccc4)c3)c2O1)N1CCNCC1 ; Clc1cc2c(c(-c3ccnc4[nH]ccc34)c1)OC(c1n[nH]c3c1CNCC3)C2 ; O=C(C1Cc2cc(Cl)cc(-c3ccnc4cc(CO)sc34)c2O1)N1CCNCC1]

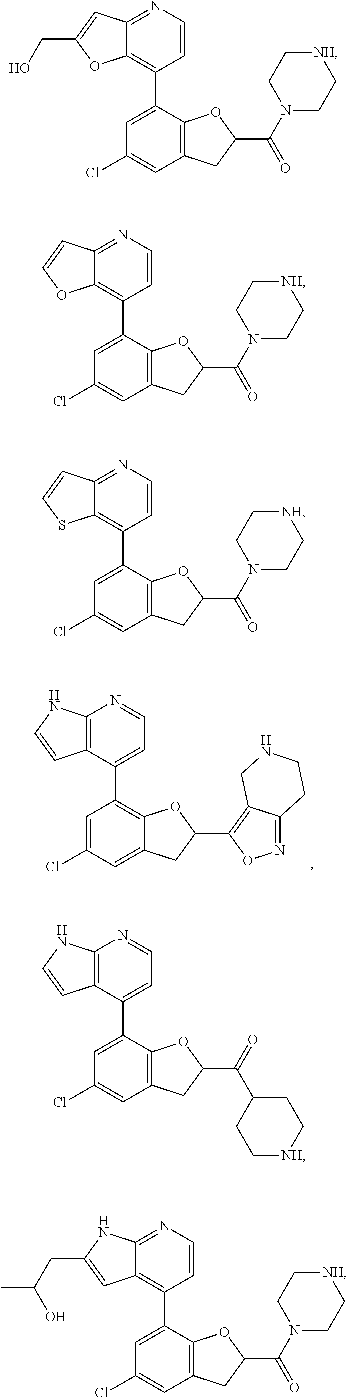 [SMILES: O=C(C1Cc2cc(Cl)cc(-c3ccnc4cc(CO)oc34)c2O1)N1CCNCC1 ; O=C(C1Cc2cc(Cl)cc(-c3ccnc4ccoc34)c2O1)N1CCNCC1 ; O=C(C1Cc2cc(Cl)cc(-c3ccnc4ccsc34)c2O1)N1CCNCC1 ; Clc1cc2c(c(-c3ccnc4[nH]ccc34)c1)OC(c1onc3c1CNCC3)C2 ; O=C(C1CCNCC1)C1Cc2cc(Cl)cc(-c3ccnc4[nH]ccc34)c2O1 ; CC(O)Cc1cc2c(-c3cc(Cl)cc4c3OC(C(=O)N3CCNCC3)C4)ccnc2[nH]1]

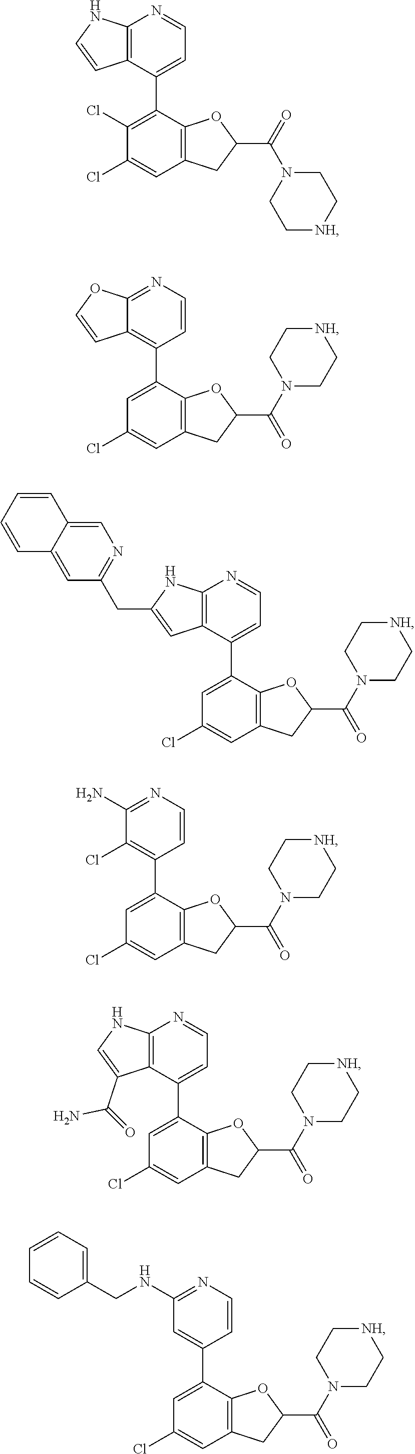 [SMILES: O=C(C1Cc2cc(Cl)c(Cl)c(-c3ccnc4[nH]ccc34)c2O1)N1CCNCC1 ; O=C(C1Cc2cc(Cl)cc(-c3ccnc4occc34)c2O1)N1CCNCC1 ; O=C(C1Cc2cc(Cl)cc(-c3ccnc4[nH]c(Cc5cc6ccccc6cn5)cc34)c2O1)N1CCNCC1 ; Nc1nccc(-c2cc(Cl)cc3c2OC(C(=O)N2CCNCC2)C3)c1Cl ; NC(=O)c1c[nH]c2nccc(-c3cc(Cl)cc4c3OC(C(=O)N3CCNCC3)C4)c12 ; O=C(C1Cc2cc(Cl)cc(-c3ccnc(NCc4ccccc4)c3)c2O1)N1CCNCC1]

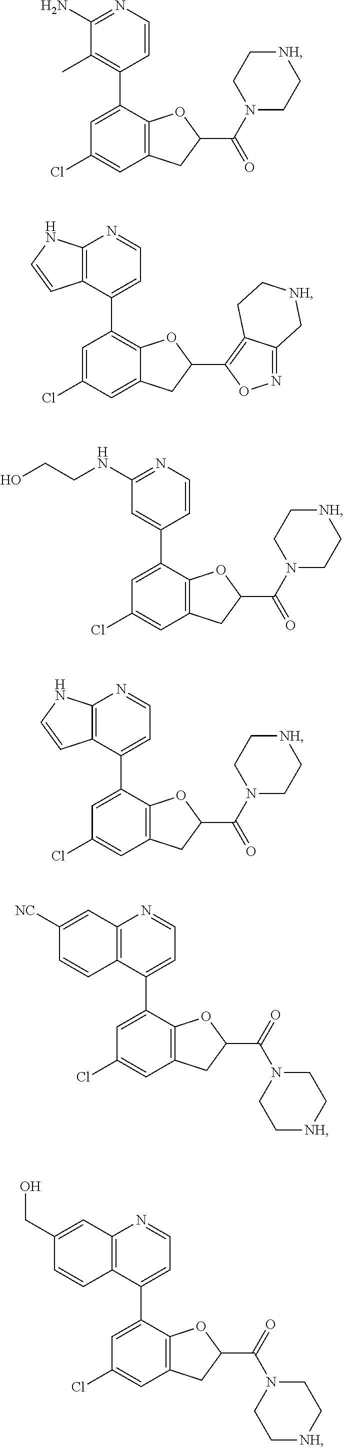 [SMILES: Cc1c(-c2cc(Cl)cc3c2OC(C(=O)N2CCNCC2)C3)ccnc1N ; Clc1cc2c(c(-c3ccnc4[nH]ccc34)c1)OC(c1onc3c1CCNC3)C2 ; O=C(C1Cc2cc(Cl)cc(-c3ccnc(NCCO)c3)c2O1)N1CCNCC1 ; O=C(C1Cc2cc(Cl)cc(-c3ccnc4[nH]ccc34)c2O1)N1CCNCC1 ; N#Cc1ccc2c(-c3cc(Cl)cc4c3OC(C(=O)N3CCNCC3)C4)ccnc2c1 ; O=C(C1Cc2cc(Cl)cc(-c3ccnc4cc(CO)ccc34)c2O1)N1CCNCC1]

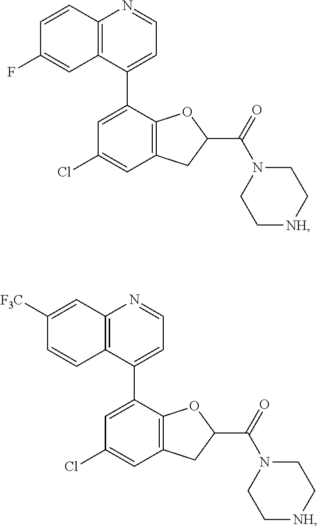 [SMILES: O=C(C1Cc2cc(Cl)cc(-c3ccnc4ccc(F)cc34)c2O1)N1CCNCC1 ; O=C(C1Cc2cc(Cl)cc(-c3ccnc4cc(C(F)(F)F)ccc34)c2O1)N1CCNCC1]

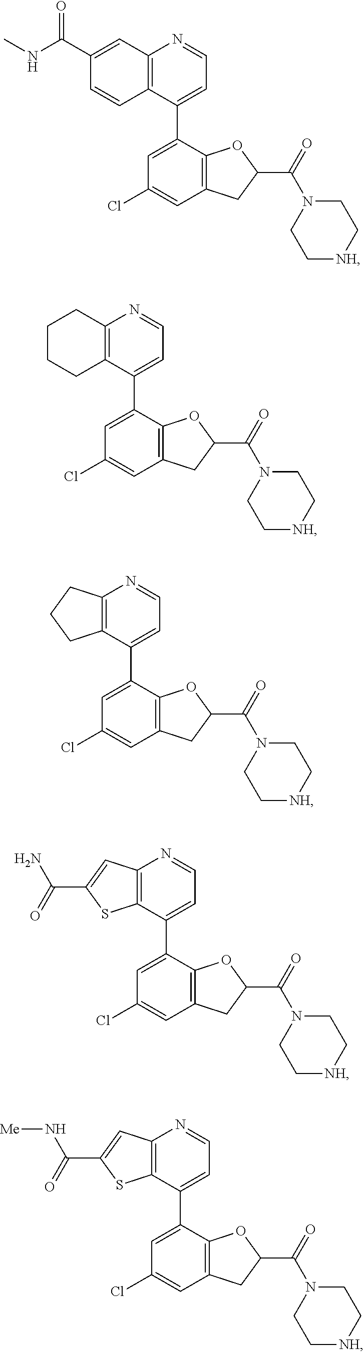 [SMILES: CNC(=O)c1ccc2c(-c3cc(Cl)cc4c3OC(C(=O)N3CCNCC3)C4)ccnc2c1 ; O=C(C1Cc2cc(Cl)cc(-c3ccnc4c3CCCC4)c2O1)N1CCNCC1 ; O=C(C1Cc2cc(Cl)cc(-c3ccnc4c3CCC4)c2O1)N1CCNCC1 ; NC(=O)c1cc2nccc(-c3cc(Cl)cc4c3OC(C(=O)N3CCNCC3)C4)c2s1 ; CNC(=O)c1cc2nccc(-c3cc(Cl)cc4c3OC(C(=O)N3CCNCC3)C4)c2s1]

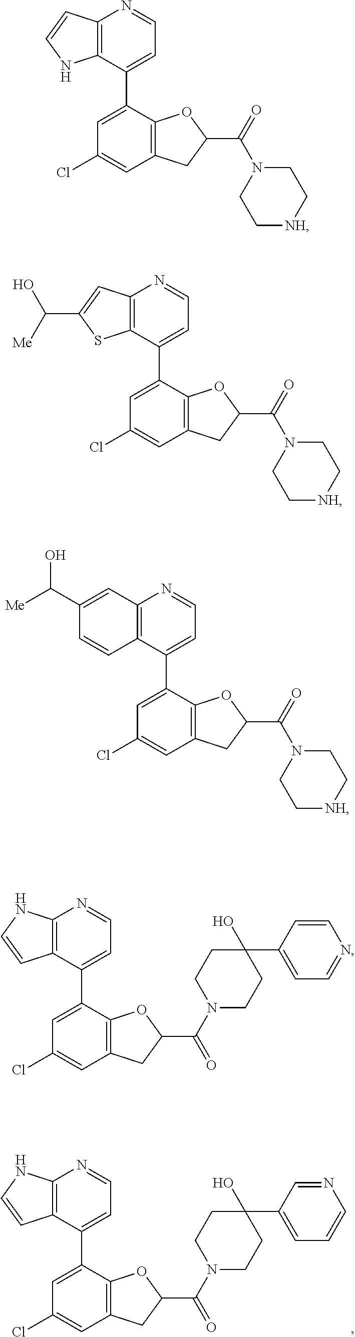 [SMILES: O=C(C1Cc2cc(Cl)cc(-c3ccnc4cc[nH]c34)c2O1)N1CCNCC1 ; CC(O)c1cc2nccc(-c3cc(Cl)cc4c3OC(C(=O)N3CCNCC3)C4)c2s1 ; CC(O)c1ccc2c(-c3cc(Cl)cc4c3OC(C(=O)N3CCNCC3)C4)ccnc2c1 ; O=C(C1Cc2cc(Cl)cc(-c3ccnc4[nH]ccc34)c2O1)N1CCC(O)(c2ccncc2)CC1 ; O=C(C1Cc2cc(Cl)cc(-c3ccnc4[nH]ccc34)c2O1)N1CCC(O)(c2cccnc2)CC1]

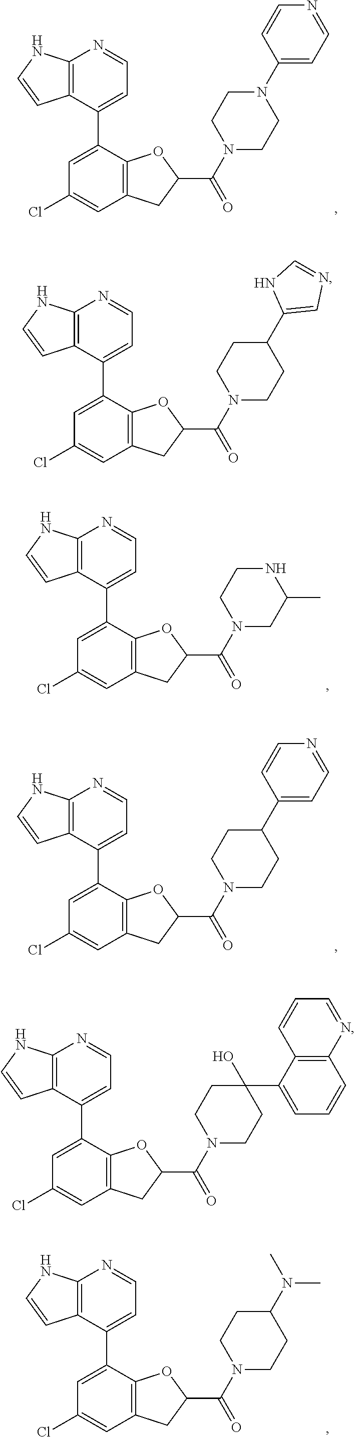 [SMILES: O=C(C1Cc2cc(Cl)cc(-c3ccnc4[nH]ccc34)c2O1)N1CCN(c2ccncc2)CC1 ; O=C(C1Cc2cc(Cl)cc(-c3ccnc4[nH]ccc34)c2O1)N1CCC(c2cnc[nH]2)CC1 ; CC1CN(C(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(c2ccncc2)CC1 ; O=C(C1Cc2cc(Cl)cc(-c3ccnc4[nH]ccc34)c2O1)N1CCC(O)(c2cccc3ncccc23)CC1 ; CN(C)C1CCN(C(=O)C2Cc3cc(Cl)cc(-c4ccnc5[nH]ccc45)c3O2)CC1]

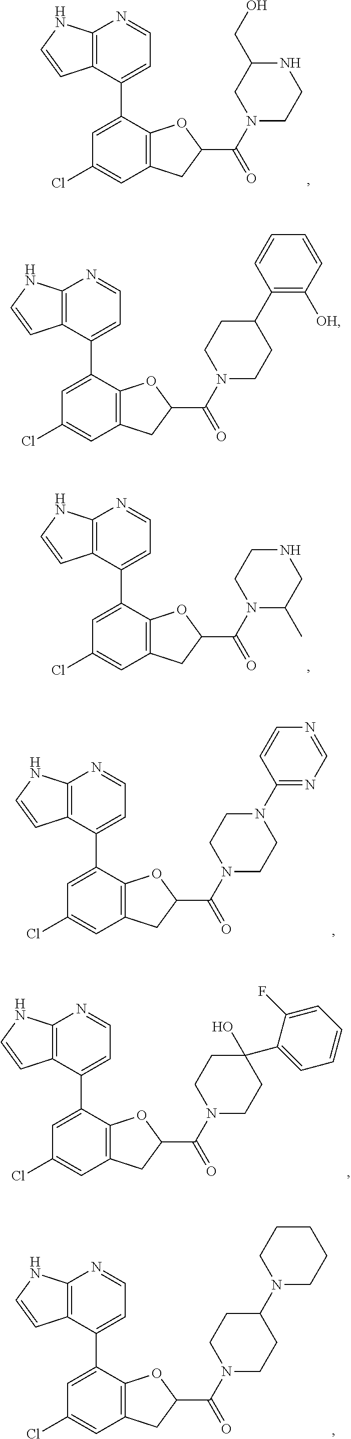 [SMILES: O=C(C1Cc2cc(Cl)cc(-c3ccnc4[nH]ccc34)c2O1)N1CCNC(CO)C1 ; O=C(C1Cc2cc(Cl)cc(-c3ccnc4[nH]ccc34)c2O1)N1CCC(c2ccccc2O)CC1 ; CC1CNCCN1C(=O)C1Cc2cc(Cl)cc(-c3ccnc4[nH]ccc34)c2O1 ; O=C(C1Cc2cc(Cl)cc(-c3ccnc4[nH]ccc34)c2O1)N1CCN(c2ccncn2)CC1 ; O=C(C1Cc2cc(Cl)cc(-c3ccnc4[nH]ccc34)c2O1)N1CCC(O)(c2ccccc2F)CC1 ; O=C(C1Cc2cc(Cl)cc(-c3ccnc4[nH]ccc34)c2O1)N1CCC(N2CCCCC2)CC1]

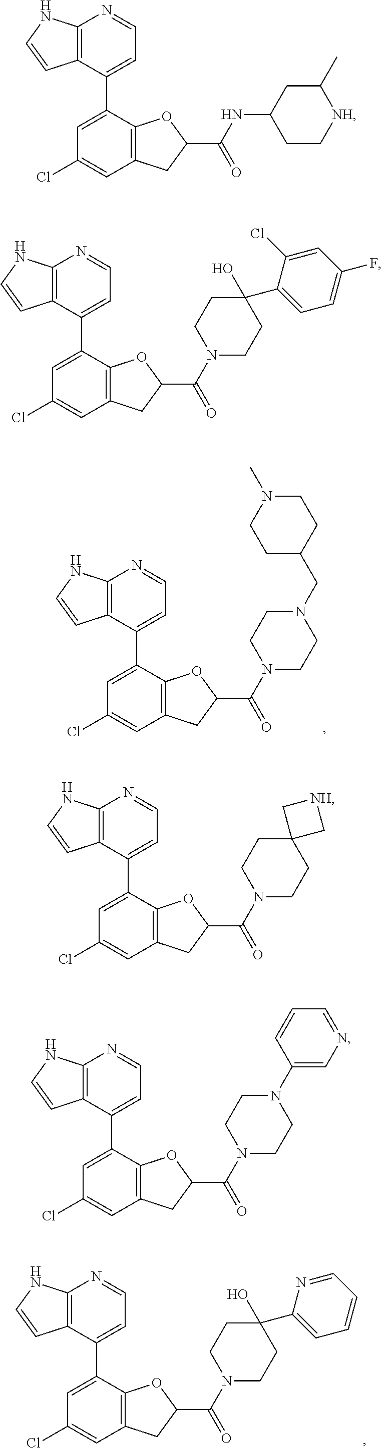 [SMILES: CC1CC(NC(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(O)(c2ccc(F)cc2Cl)CC1 ; CN1CCC(CN2CCN(C(=O)C3Cc4cc(Cl)cc(-c5ccnc6[nH]ccc56)c4O3)CC2)CC1 ; O=C(C1Cc2cc(Cl)cc(-c3ccnc4[nH]ccc34)c2O1)N1CCC2(CC1)CNC2 ; O=C(C1Cc2cc(Cl)cc(-c3ccnc4[nH]ccc34)c2O1)N1CCN(c2cccnc2)CC1 ; O=C(C1Cc2cc(Cl)cc(-c3ccnc4[nH]ccc34)c2O1)N1CCC(O)(c2ccccn2)CC1]

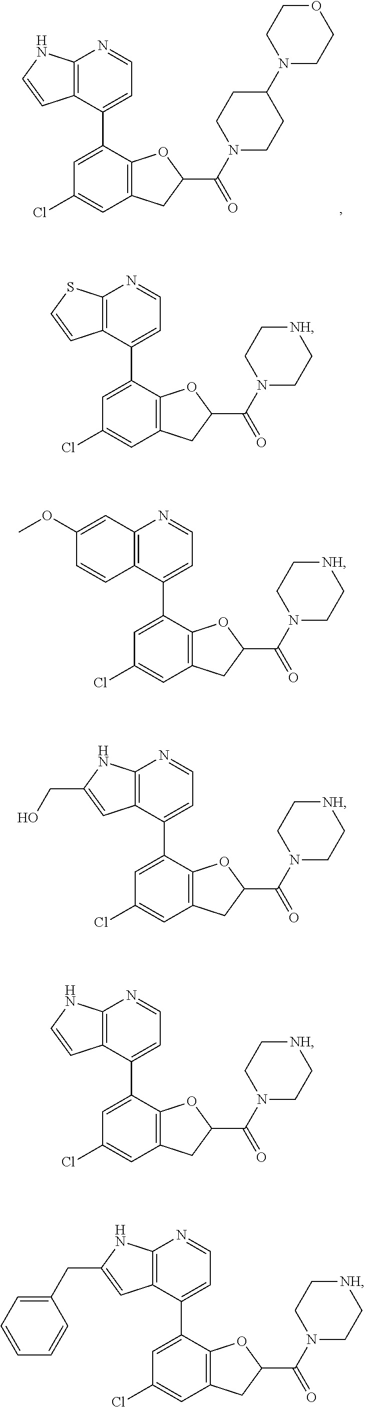 [SMILES: O=C(C1Cc2cc(Cl)cc(-c3ccnc4[nH]ccc34)c2O1)N1CCC(N2CCOCC2)CC1 ; O=C(C1Cc2cc(Cl)cc(-c3ccnc4sccc34)c2O1)N1CCNCC1 ; COc1ccc2c(-c3cc(Cl)cc4c3OC(C(=O)N3CCNCC3)C4)ccnc2c1 ; O=C(C1Cc2cc(Cl)cc(-c3ccnc4[nH]c(CO)cc34)c2O1)N1CCNCC1 ; O=C(C1Cc2cc(Cl)cc(-c3ccnc4[nH]ccc34)c2O1)N1CCNCC1 ; O=C(C1Cc2cc(Cl)cc(-c3ccnc4[nH]c(Cc5ccccc5)cc34)c2O1)N1CCNCC1]

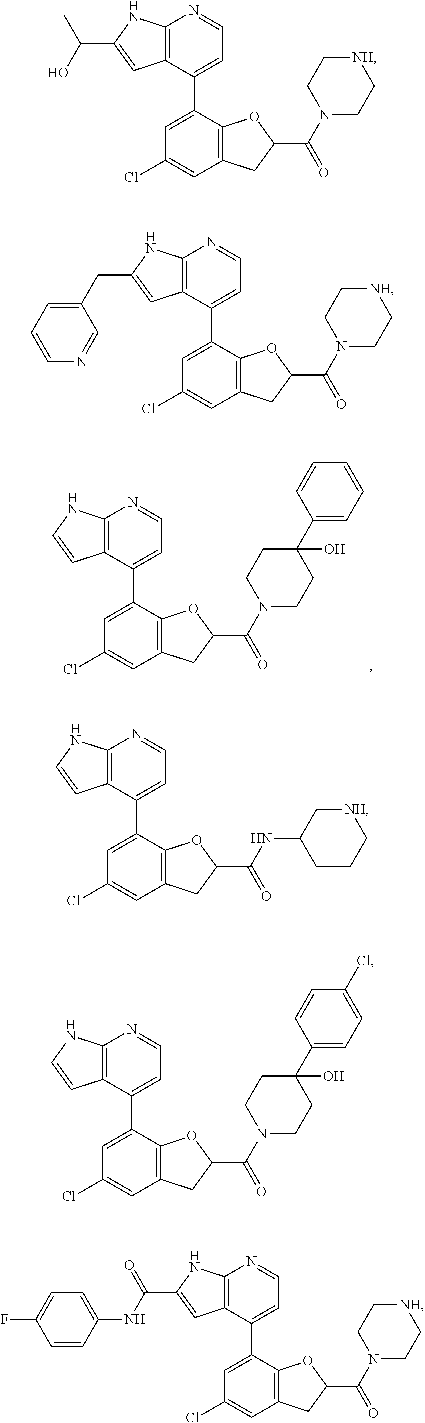 [SMILES: CC(O)c1cc2c(-c3cc(Cl)cc4c3OC(C(=O)N3CCNCC3)C4)ccnc2[nH]1 ; O=C(C1Cc2cc(Cl)cc(-c3ccnc4[nH]c(Cc5cccnc5)cc34)c2O1)N1CCNCC1 ; O=C(C1Cc2cc(Cl)cc(-c3ccnc4[nH]ccc34)c2O1)N1CCC(O)(c2ccccc2)CC1 ; O=C(NC1CCCNC1)C1Cc2cc(Cl)cc(-c3ccnc4[nH]ccc34)c2O1 ; O=C(C1Cc2cc(Cl)cc(-c3ccnc4[nH]ccc34)c2O1)N1CCC(O)(c2ccc(Cl)cc2)CC1 ; O=C(Nc1ccc(F)cc1)c1cc2c(-c3cc(Cl)cc4c3OC(C(=O)N3CCNCC3)C4)ccnc2[nH]1]

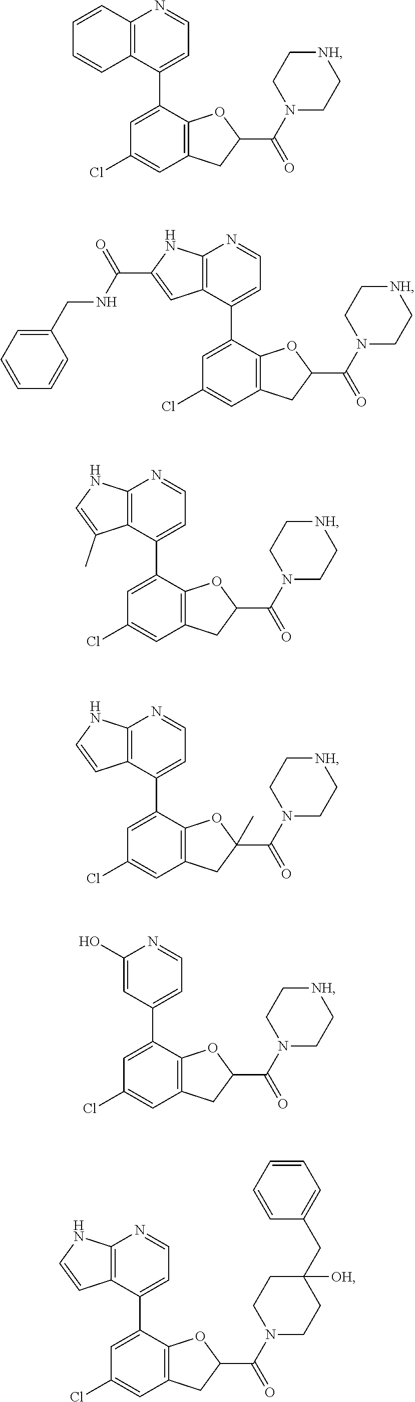 [SMILES: O=C(C1Cc2cc(Cl)cc(-c3ccnc4ccccc34)c2O1)N1CCNCC1 ; O=C(NCc1ccccc1)c1cc2c(-c3cc(Cl)cc4c3OC(C(=O)N3CCNCC3)C4)ccnc2[nH]1 ; Cc1c[nH]c2nccc(-c3cc(Cl)cc4c3OC(C(=O)N3CCNCC3)C4)c12 ; CC1(C(=O)N2CCNCC2)Cc2cc(Cl)cc(-c3ccnc4[nH]ccc34)c2O1 ; O=C(C1Cc2cc(Cl)cc(-c3ccnc(O)c3)c2O1)N1CCNCC1 ; O=C(C1Cc2cc(Cl)cc(-c3ccnc4[nH]ccc34)c2O1)N1CCC(O)(Cc2ccccc2)CC1]

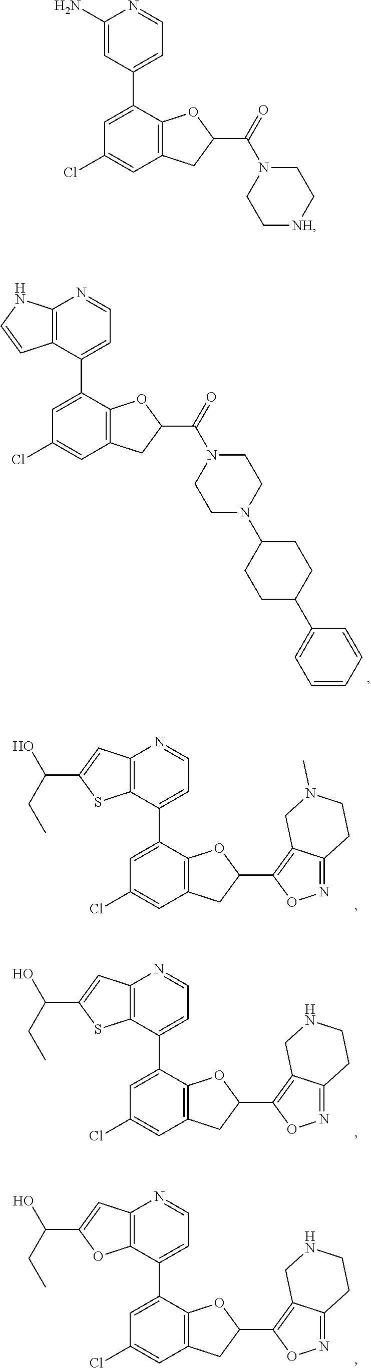 [SMILES: Nc1cc(-c2cc(Cl)cc3c2OC(C(=O)N2CCNCC2)C3)ccn1 ; O=C(C1Cc2cc(Cl)cc(-c3ccnc4[nH]ccc34)c2O1)N1CCN(C2CCC(c3ccccc3)CC2)CC1 ; CCC(O)c1cc2nccc(-c3cc(Cl)cc4c3OC(c3onc5c3CN(C)CC5)C4)c2s1 ; CCC(O)c1cc2nccc(-c3cc(Cl)cc4c3OC(c3onc5c3CNCC5)C4)c2s1 ; CCC(O)c1cc2nccc(-c3cc(Cl)cc4c3OC(c3onc5c3CNCC5)C4)c2o1]

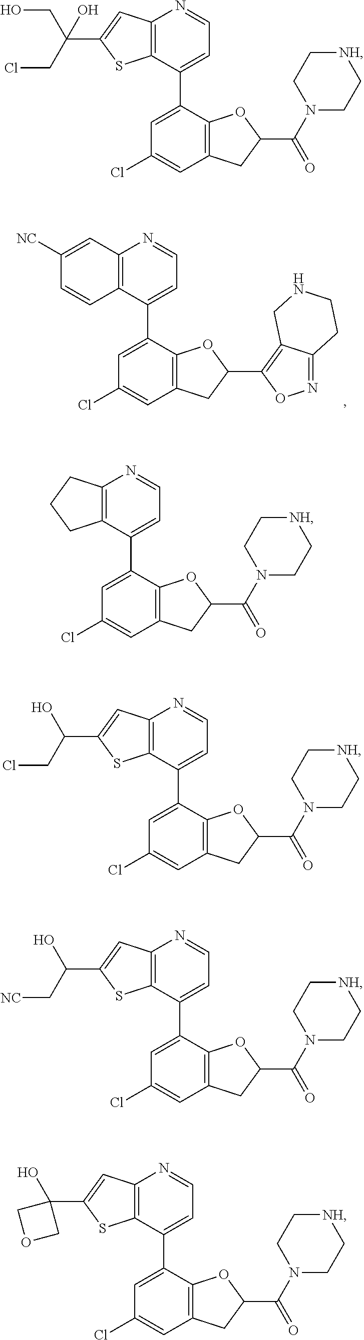 [SMILES: O=C(C1Cc2cc(Cl)cc(-c3ccnc4cc(C(O)(CO)CCl)sc34)c2O1)N1CCNCC1 ; N#Cc1ccc2c(-c3cc(Cl)cc4c3OC(c3onc5c3CNCC5)C4)ccnc2c1 ; O=C(C1Cc2cc(Cl)cc(-c3ccnc4c3CCC4)c2O1)N1CCNCC1 ; O=C(C1Cc2cc(Cl)cc(-c3ccnc4cc(C(O)CCl)sc34)c2O1)N1CCNCC1 ; N#CCC(O)c1cc2nccc(-c3cc(Cl)cc4c3OC(C(=O)N3CCNCC3)C4)c2s1 ; O=C(C1Cc2cc(Cl)cc(-c3ccnc4cc(C5(O)COC5)sc34)c2O1)N1CCNCC1]

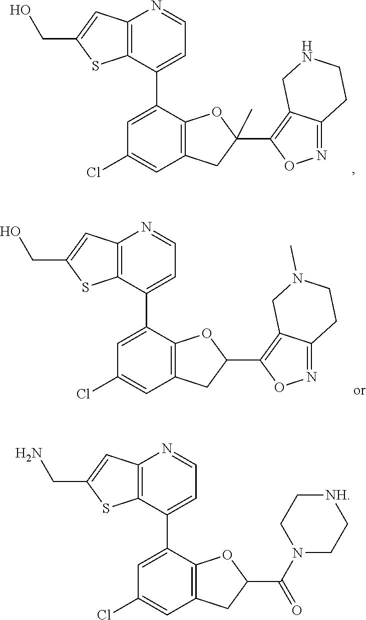 [SMILES: CC1(c2onc3c2CNCC3)Cc2cc(Cl)cc(-c3ccnc4cc(CO)sc34)c2O1 ; CN1CCc2noc(C3Cc4cc(Cl)cc(-c5ccnc6cc(CO)sc56)c4O3)c2C1 ; NCc1cc2nccc(-c3cc(Cl)cc4c3OC(C(=O)N3CCNCC3)C4)c2s1]

In embodiments, the compound is

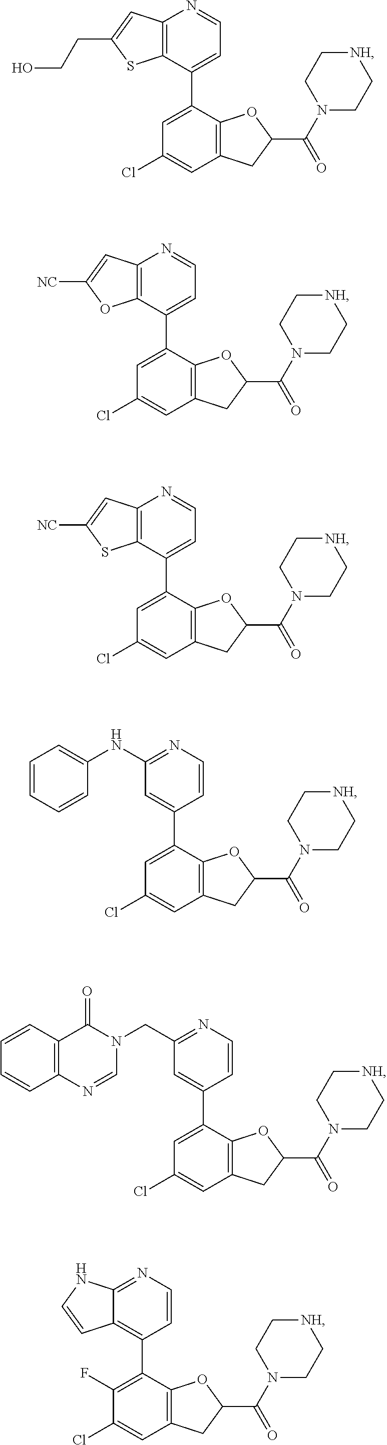 [SMILES: O=C(C1Cc2cc(Cl)cc(-c3ccnc4cc(CCO)sc34)c2O1)N1CCNCC1 ; N#Cc1cc2nccc(-c3cc(Cl)cc4c3OC(C(=O)N3CCNCC3)C4)c2o1 ; N#Cc1cc2nccc(-c3cc(Cl)cc4c3OC(C(=O)N3CCNCC3)C4)c2s1 ; O=C(C1Cc2cc(Cl)cc(-c3ccnc(Nc4ccccc4)c3)c2O1)N1CCNCC1 ; O=C(C1Cc2cc(Cl)cc(-c3ccnc(Cn4cnc5ccccc5c4=O)c3)c2O1)N1CCNCC1 ; O=C(C1Cc2cc(Cl)c(F)c(-c3ccnc4[nH]ccc34)c2O1)N1CCNCC1]

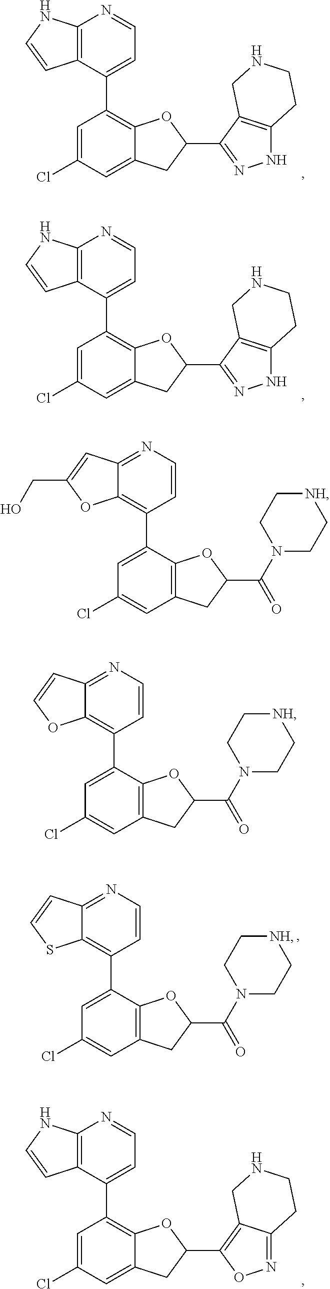 [SMILES: Clc1cc2c(c(-c3ccnc4[nH]ccc34)c1)OC(c1n[nH]c3c1CNCC3)C2 ; Clc1cc2c(c(-c3ccnc4[nH]ccc34)c1)OC(c1n[nH]c3c1CNCC3)C2 ; O=C(C1Cc2cc(Cl)cc(-c3ccnc4cc(CO)oc34)c2O1)N1CCNCC1 ; O=C(C1Cc2cc(Cl)cc(-c3ccnc4ccoc34)c2O1)N1CCNCC1 ; O=C(C1Cc2cc(Cl)cc(-c3ccnc4ccsc34)c2O1)N1CCNCC1 ; Clc1cc2c(c(-c3ccnc4[nH]ccc34)c1)OC(c1onc3c1CNCC3)C2]

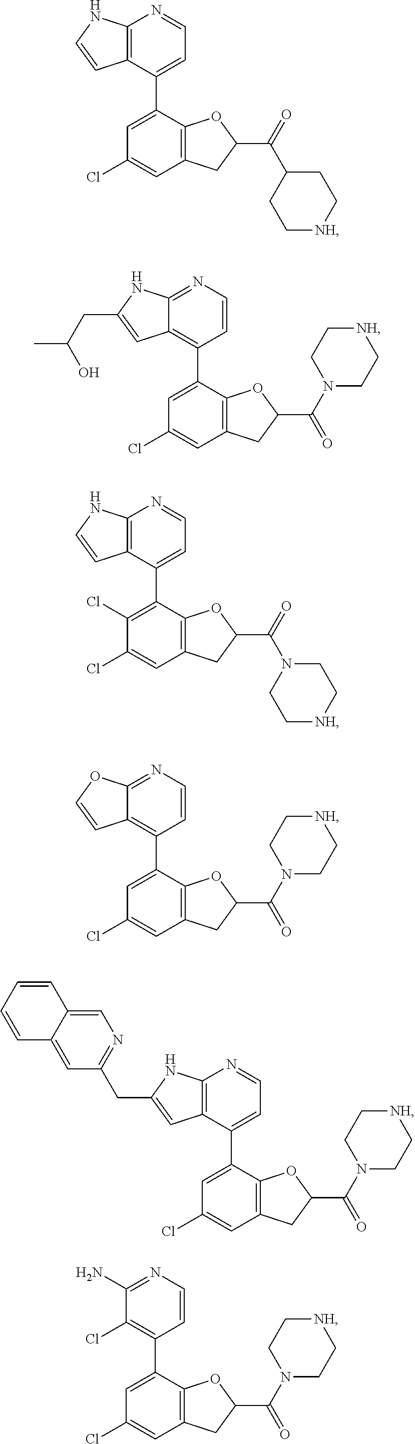

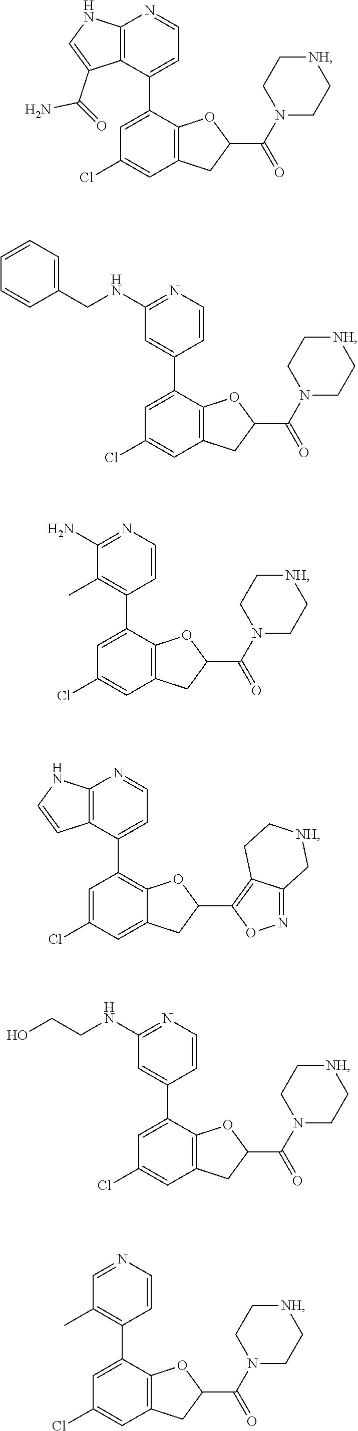 [SMILES: NC(=O)c1c[nH]c2nccc(-c3cc(Cl)cc4c3OC(C(=O)N3CCNCC3)C4)c12 ; O=C(C1Cc2cc(Cl)cc(-c3ccnc(NCc4ccccc4)c3)c2O1)N1CCNCC1 ; Cc1c(-c2cc(Cl)cc3c2OC(C(=O)N2CCNCC2)C3)ccnc1N ; Clc1cc2c(c(-c3ccnc4[nH]ccc34)c1)OC(c1onc3c1CCNC3)C2 ; O=C(C1Cc2cc(Cl)cc(-c3ccnc(NCCO)c3)c2O1)N1CCNCC1 ; Cc1cnccc1-c1cc(Cl)cc2c1OC(C(=O)N1CCNCC1)C2]

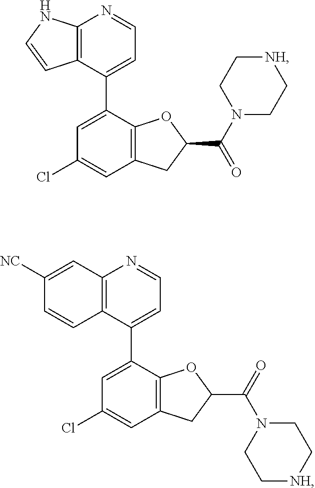 [SMILES: O=C([C@H]1Cc2cc(Cl)cc(-c3ccnc4[nH]ccc34)c2O1)N1CCNCC1 ; N#Cc1ccc2c(-c3cc(Cl)cc4c3OC(C(=O)N3CCNCC3)C4)ccnc2c1]

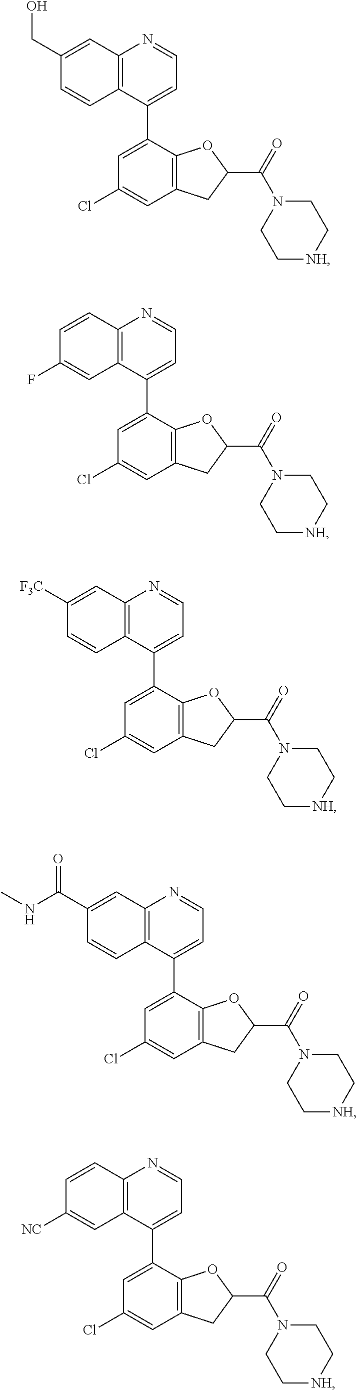 [SMILES: O=C(C1Cc2cc(Cl)cc(-c3ccnc4cc(CO)ccc34)c2O1)N1CCNCC1 ; O=C(C1Cc2cc(Cl)cc(-c3ccnc4ccc(F)cc34)c2O1)N1CCNCC1 ; O=C(C1Cc2cc(Cl)cc(-c3ccnc4cc(C(F)(F)F)ccc34)c2O1)N1CCNCC1 ; CNC(=O)c1ccc2c(-c3cc(Cl)cc4c3OC(C(=O)N3CCNCC3)C4)ccnc2c1 ; N#Cc1ccc2nccc(-c3cc(Cl)cc4c3OC(C(=O)N3CCNCC3)C4)c2c1]

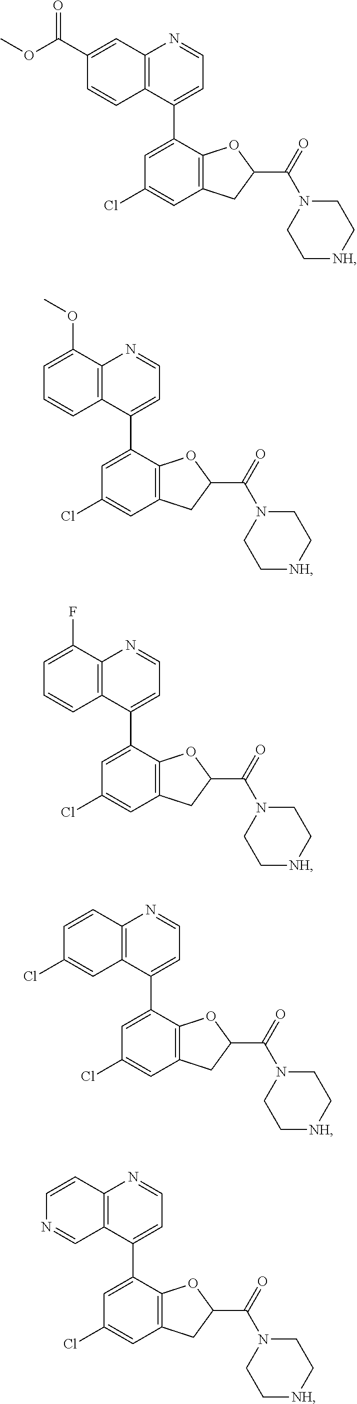 [SMILES: COC(=O)c1ccc2c(-c3cc(Cl)cc4c3OC(C(=O)N3CCNCC3)C4)ccnc2c1 ; COc1cccc2c(-c3cc(Cl)cc4c3OC(C(=O)N3CCNCC3)C4)ccnc12 ; O=C(C1Cc2cc(Cl)cc(-c3ccnc4c(F)cccc34)c2O1)N1CCNCC1 ; O=C(C1Cc2cc(Cl)cc(-c3ccnc4ccc(Cl)cc34)c2O1)N1CCNCC1 ; O=C(C1Cc2cc(Cl)cc(-c3ccnc4ccncc34)c2O1)N1CCNCC1]

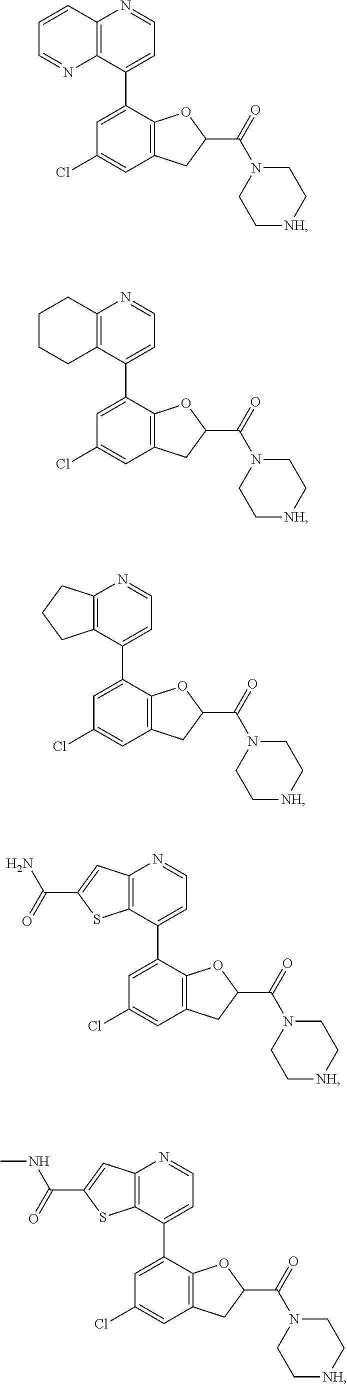 [SMILES: O=C(C1Cc2cc(Cl)cc(-c3ccnc4cccnc34)c2O1)N1CCNCC1 ; O=C(C1Cc2cc(Cl)cc(-c3ccnc4c3CCCC4)c2O1)N1CCNCC1 ; O=C(C1Cc2cc(Cl)cc(-c3ccnc4c3CCC4)c2O1)N1CCNCC1 ; NC(=O)c1cc2nccc(-c3cc(Cl)cc4c3OC(C(=O)N3CCNCC3)C4)c2s1 ; CNC(=O)c1cc2nccc(-c3cc(Cl)cc4c3OC(C(=O)N3CCNCC3)C4)c2s1]

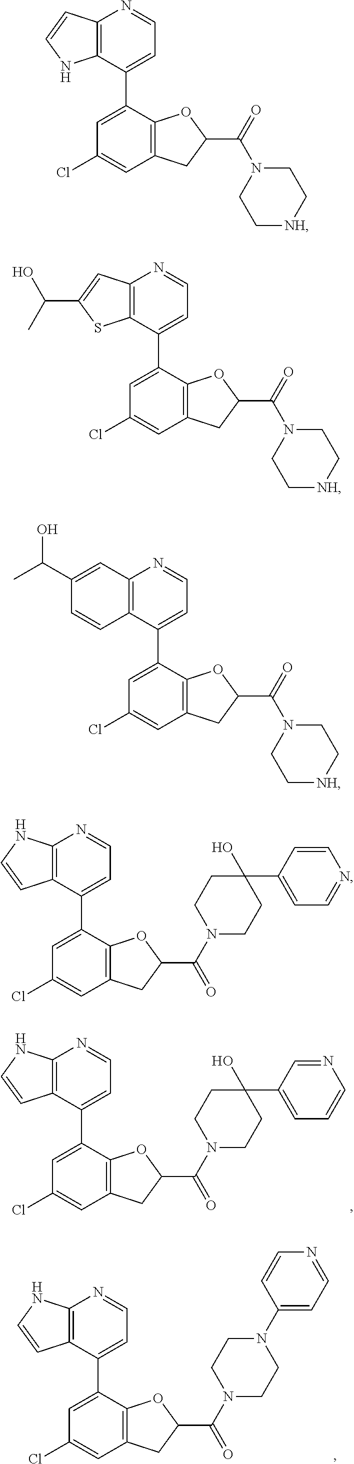 [SMILES: O=C(C1Cc2cc(Cl)cc(-c3ccnc4cc[nH]c34)c2O1)N1CCNCC1 ; CC(O)c1cc2nccc(-c3cc(Cl)cc4c3OC(C(=O)N3CCNCC3)C4)c2s1 ; CC(O)c1ccc2c(-c3cc(Cl)cc4c3OC(C(=O)N3CCNCC3)C4)ccnc2c1 ; O=C(C1Cc2cc(Cl)cc(-c3ccnc4[nH]ccc34)c2O1)N1CCC(O)(c2ccncc2)CC1 ; O=C(C1Cc2cc(Cl)cc(-c3ccnc4[nH]ccc34)c2O1)N1CCC(O)(c2cccnc2)CC1 ; O=C(C1Cc2cc(Cl)cc(-c3ccnc4[nH]ccc34)c2O1)N1CCN(c2ccncc2)CC1]

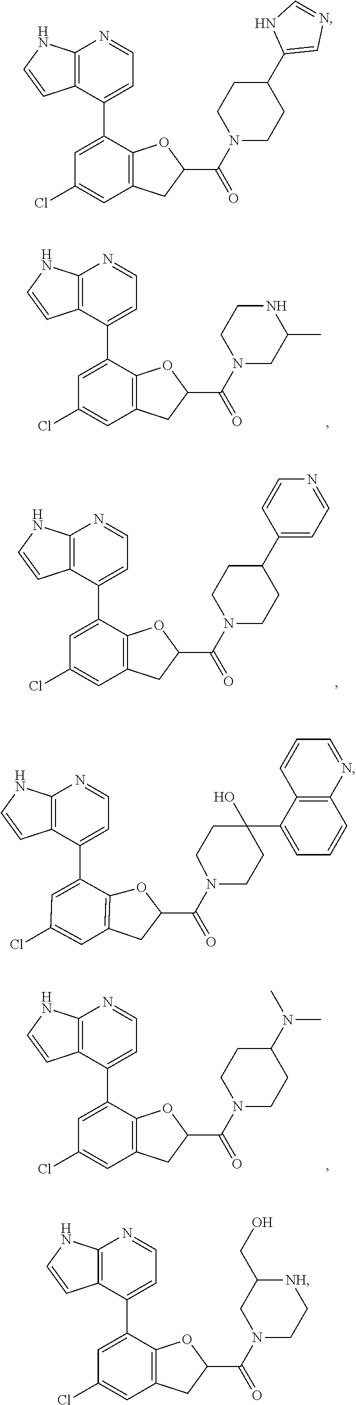 [SMILES: O=C(C1Cc2cc(Cl)cc(-c3ccnc4[nH]ccc34)c2O1)N1CCC(c2cnc[nH]2)CC1 ; CC1CN(C(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(c2ccncc2)CC1 ; O=C(C1Cc2cc(Cl)cc(-c3ccnc4[nH]ccc34)c2O1)N1CCC(O)(c2cccc3ncccc23)CC1 ; CN(C)C1CCN(C(=O)C2Cc3cc(Cl)cc(-c4ccnc5[nH]ccc45)c3O2)CC1 ; O=C(C1Cc2cc(Cl)cc(-c3ccnc4[nH]ccc34)c2O1)N1CCNC(CO)C1]

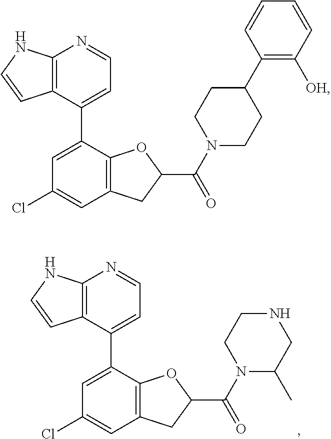 [SMILES: O=C(C1Cc2cc(Cl)cc(-c3ccnc4[nH]ccc34)c2O1)N1CCC(c2ccccc2O)CC1 ; CC1CNCCN1C(=O)C1Cc2cc(Cl)cc(-c3ccnc4[nH]ccc34)c2O1]

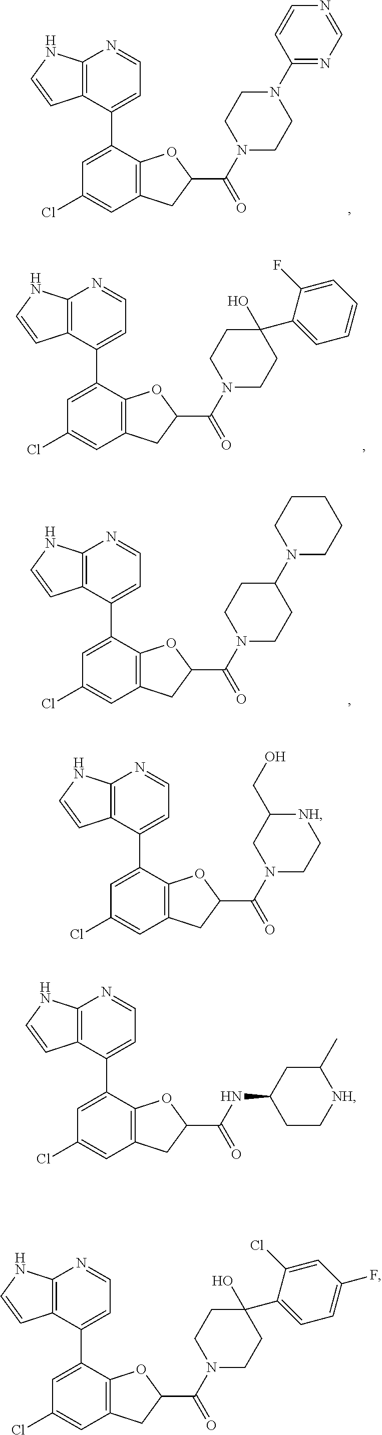 [SMILES: O=C(C1Cc2cc(Cl)cc(-c3ccnc4[nH]ccc34)c2O1)N1CCN(c2ccncn2)CC1 ; O=C(C1Cc2cc(Cl)cc(-c3ccnc4[nH]ccc34)c2O1)N1CCC(O)(c2ccccc2F)CC1 ; O=C(C1Cc2cc(Cl)cc(-c3ccnc4[nH]ccc34)c2O1)N1CCC(N2CCCCC2)CC1 ; O=C(C1Cc2cc(Cl)cc(-c3ccnc4[nH]ccc34)c2O1)N1CCNC(CO)C1 ; CC1C[C@H](NC(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(O)(c2ccc(F)cc2Cl)CC1]

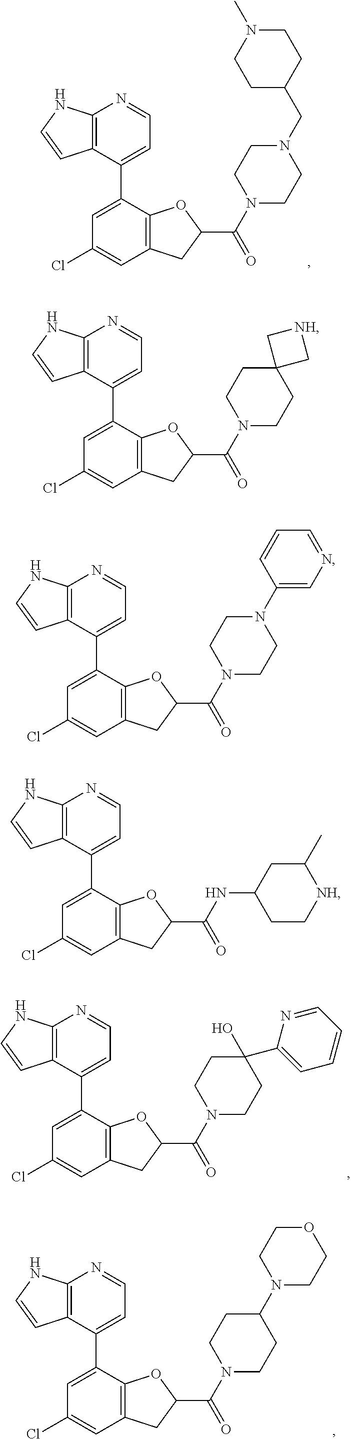 [SMILES: CN1CCC(CN2CCN(C(=O)C3Cc4cc(Cl)cc(-c5ccnc6[nH]ccc56)c4O3)CC2)CC1 ; O=C(C1Cc2cc(Cl)cc(-c3ccnc4[nH]ccc34)c2O1)N1CCC2(CC1)CNC2 ; O=C(C1Cc2cc(Cl)cc(-c3ccnc4[nH]ccc34)c2O1)N1CCN(c2cccnc2)CC1 ; CC1CC(NC(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(O)(c2ccccn2)CC1 ; O=C(C1Cc2cc(Cl)cc(-c3ccnc4[nH]ccc34)c2O1)N1CCC(N2CCOCC2)CC1]

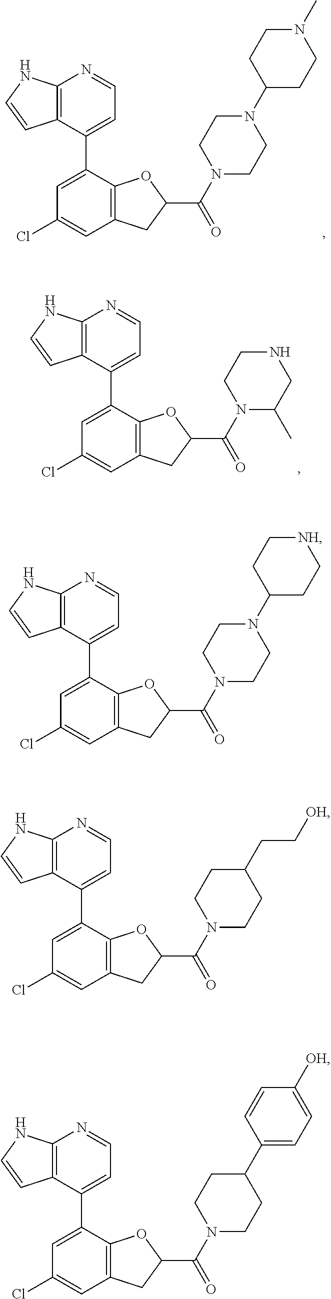 [SMILES: CN1CCC(N2CCN(C(=O)C3Cc4cc(Cl)cc(-c5ccnc6[nH]ccc56)c4O3)CC2)CC1 ; CC1CNCCN1C(=O)C1Cc2cc(Cl)cc(-c3ccnc4[nH]ccc34)c2O1 ; O=C(C1Cc2cc(Cl)cc(-c3ccnc4[nH]ccc34)c2O1)N1CCN(C2CCNCC2)CC1 ; O=C(C1Cc2cc(Cl)cc(-c3ccnc4[nH]ccc34)c2O1)N1CCC(CCO)CC1 ; O=C(C1Cc2cc(Cl)cc(-c3ccnc4[nH]ccc34)c2O1)N1CCC(c2ccc(O)cc2)CC1]

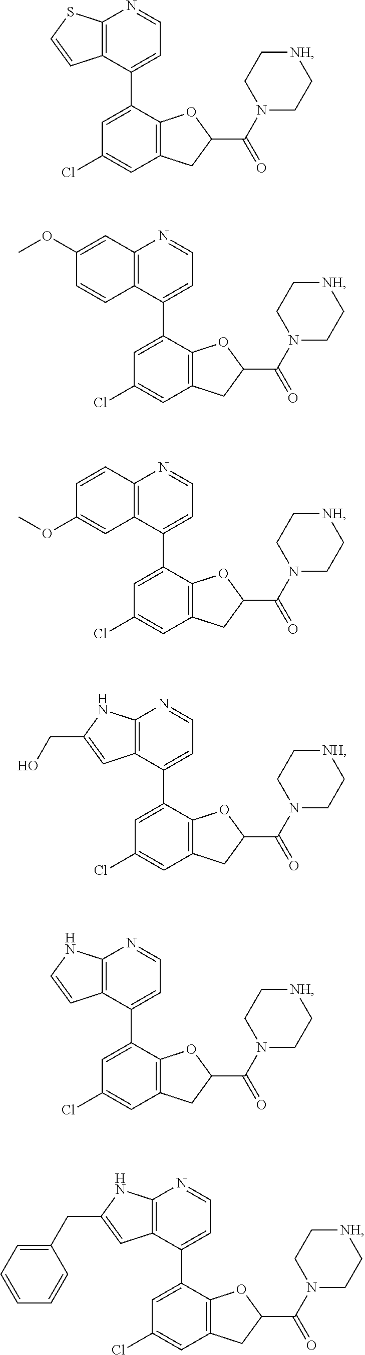 [SMILES: O=C(C1Cc2cc(Cl)cc(-c3ccnc4sccc34)c2O1)N1CCNCC1 ; COc1ccc2c(-c3cc(Cl)cc4c3OC(C(=O)N3CCNCC3)C4)ccnc2c1 ; COc1ccc2nccc(-c3cc(Cl)cc4c3OC(C(=O)N3CCNCC3)C4)c2c1 ; O=C(C1Cc2cc(Cl)cc(-c3ccnc4[nH]c(CO)cc34)c2O1)N1CCNCC1 ; O=C(C1Cc2cc(Cl)cc(-c3ccnc4[nH]ccc34)c2O1)N1CCNCC1 ; O=C(C1Cc2cc(Cl)cc(-c3ccnc4[nH]c(Cc5ccccc5)cc34)c2O1)N1CCNCC1]

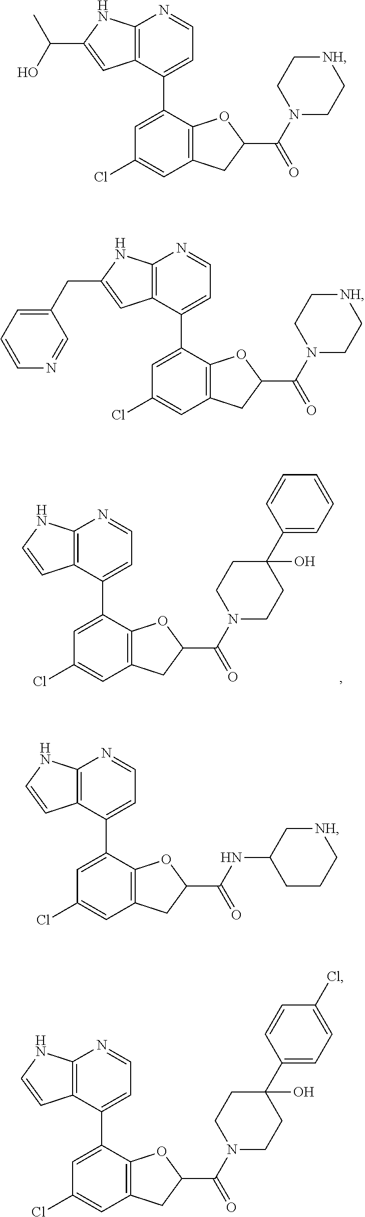 [SMILES: CC(O)c1cc2c(-c3cc(Cl)cc4c3OC(C(=O)N3CCNCC3)C4)ccnc2[nH]1 ; O=C(C1Cc2cc(Cl)cc(-c3ccnc4[nH]c(Cc5cccnc5)cc34)c2O1)N1CCNCC1 ; O=C(C1Cc2cc(Cl)cc(-c3ccnc4[nH]ccc34)c2O1)N1CCC(O)(c2ccccc2)CC1 ; O=C(NC1CCCNC1)C1Cc2cc(Cl)cc(-c3ccnc4[nH]ccc34)c2O1 ; O=C(C1Cc2cc(Cl)cc(-c3ccnc4[nH]ccc34)c2O1)N1CCC(O)(c2ccc(Cl)cc2)CC1]

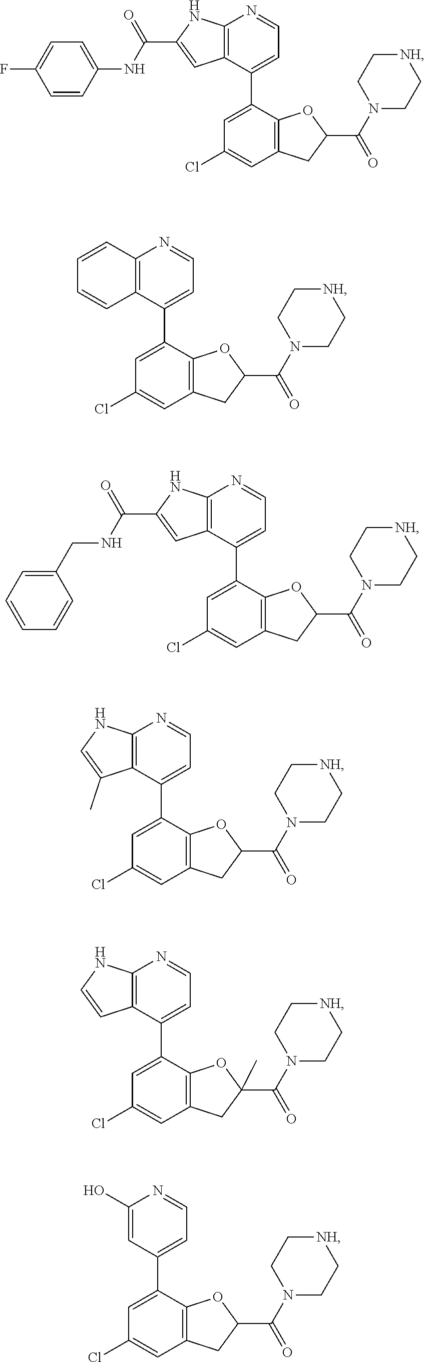 [SMILES: O=C(Nc1ccc(F)cc1)c1cc2c(-c3cc(Cl)cc4c3OC(C(=O)N3CCNCC3)C4)ccnc2[nH]1 ; O=C(C1Cc2cc(Cl)cc(-c3ccnc4ccccc34)c2O1)N1CCNCC1 ; O=C(NCc1ccccc1)c1cc2c(-c3cc(Cl)cc4c3OC(C(=O)N3CCNCC3)C4)ccnc2[nH]1 ; Cc1c[nH]c2nccc(-c3cc(Cl)cc4c3OC(C(=O)N3CCNCC3)C4)c12 ; CC1(C(=O)N2CCNCC2)Cc2cc(Cl)cc(-c3ccnc4[nH]ccc34)c2O1 ; O=C(C1Cc2cc(Cl)cc(-c3ccnc(O)c3)c2O1)N1CCNCC1]

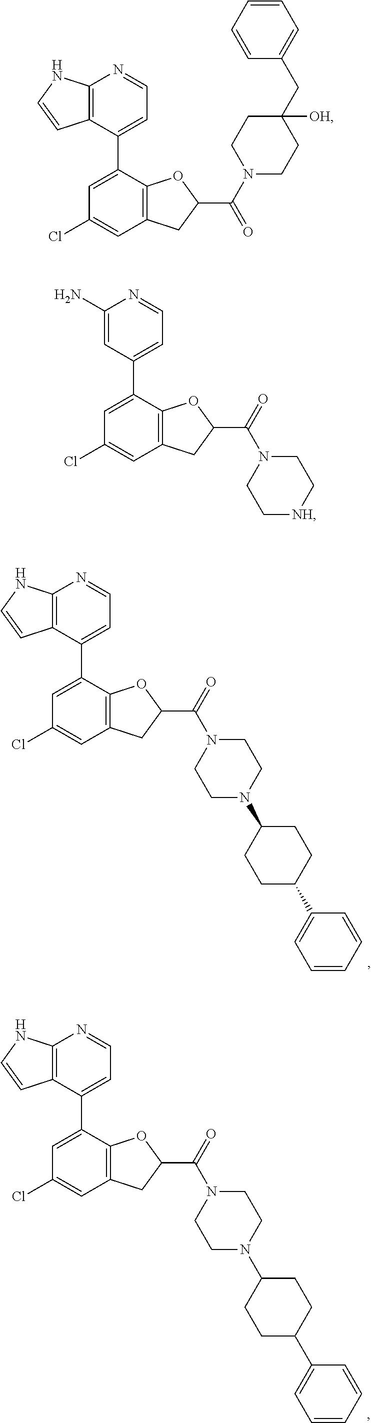 [SMILES: O=C(C1Cc2cc(Cl)cc(-c3ccnc4[nH]ccc34)c2O1)N1CCC(O)(Cc2ccccc2)CC1 ; Nc1cc(-c2cc(Cl)cc3c2OC(C(=O)N2CCNCC2)C3)ccn1 ; O=C(C1Cc2cc(Cl)cc(-c3ccnc4[nH]ccc34)c2O1)N1CCN([C@H]2CC[C@H](c3ccccc3)CC2)CC1 ; O=C(C1Cc2cc(Cl)cc(-c3ccnc4[nH]ccc34)c2O1)N1CCN(C2CCC(c3ccccc3)CC2)CC1]

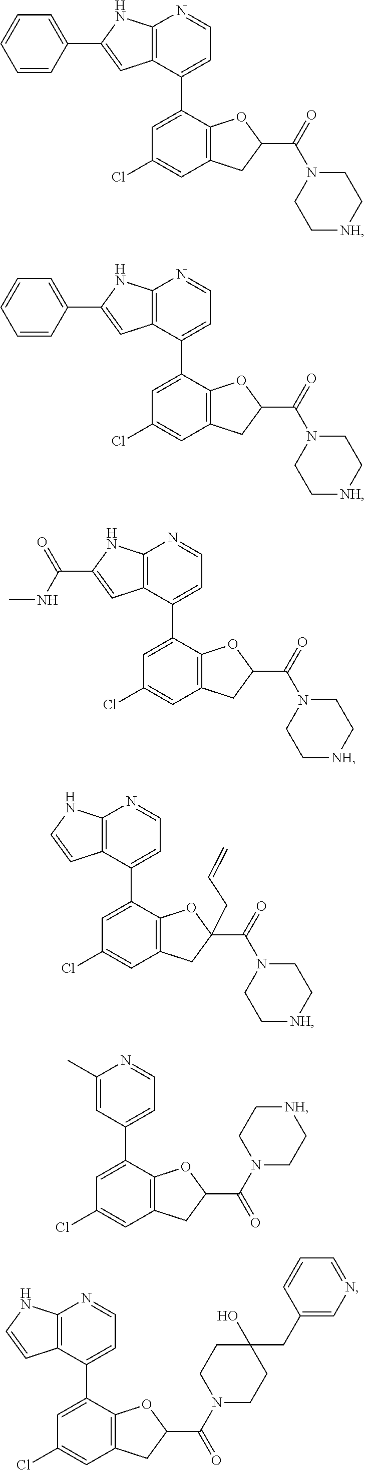 [SMILES: O=C(C1Cc2cc(Cl)cc(-c3ccnc4[nH]c(-c5ccccc5)cc34)c2O1)N1CCNCC1 ; O=C(C1Cc2cc(Cl)cc(-c3ccnc4[nH]c(-c5ccccc5)cc34)c2O1)N1CCNCC1 ; CNC(=O)c1cc2c(-c3cc(Cl)cc4c3OC(C(=O)N3CCNCC3)C4)ccnc2[nH]1 ; C=CCC1(C(=O)N2CCNCC2)Cc2cc(Cl)cc(-c3ccnc4[nH]ccc34)c2O1 ; Cc1cc(-c2cc(Cl)cc3c2OC(C(=O)N2CCNCC2)C3)ccn1 ; O=C(C1Cc2cc(Cl)cc(-c3ccnc4[nH]ccc34)c2O1)N1CCC(O)(Cc2cccnc2)CC1]

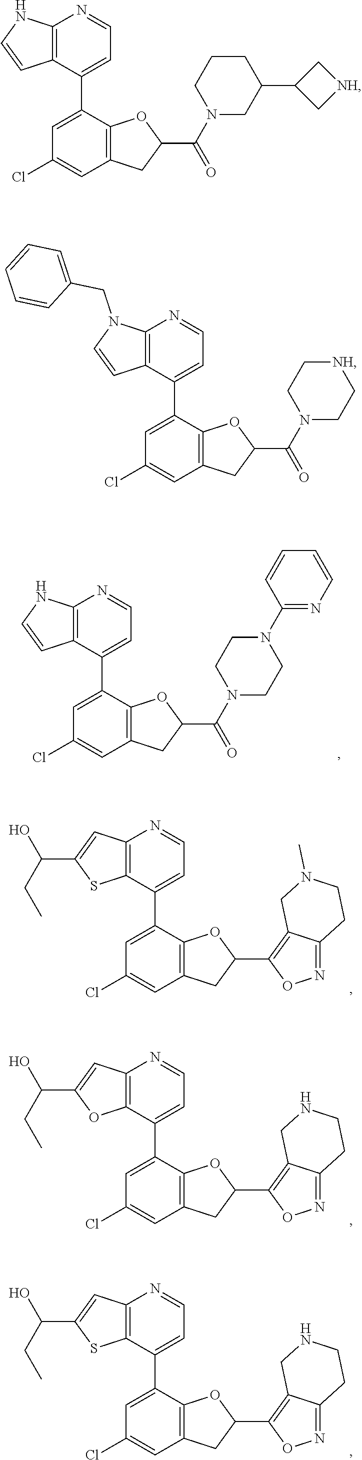 [SMILES: O=C(C1Cc2cc(Cl)cc(-c3ccnc4[nH]ccc34)c2O1)N1CCCC(C2CNC2)C1 ; O=C(C1Cc2cc(Cl)cc(-c3ccnc4c3ccn4Cc3ccccc3)c2O1)N1CCNCC1 ; O=C(C1Cc2cc(Cl)cc(-c3ccnc4[nH]ccc34)c2O1)N1CCN(c2ccccn2)CC1 ; CCC(O)c1cc2nccc(-c3cc(Cl)cc4c3OC(c3onc5c3CN(C)CC5)C4)c2s1 ; CCC(O)c1cc2nccc(-c3cc(Cl)cc4c3OC(c3onc5c3CNCC5)C4)c2o1 ; CCC(O)c1cc2nccc(-c3cc(Cl)cc4c3OC(c3onc5c3CNCC5)C4)c2s1]

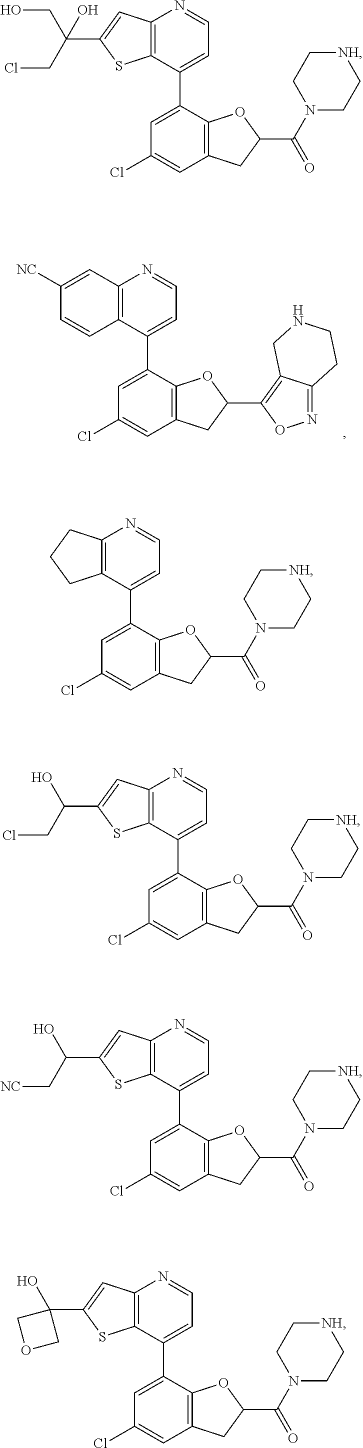 [SMILES: O=C(C1Cc2cc(Cl)cc(-c3ccnc4cc(C(O)(CO)CCl)sc34)c2O1)N1CCNCC1 ; N#Cc1ccc2c(-c3cc(Cl)cc4c3OC(c3onc5c3CNCC5)C4)ccnc2c1 ; O=C(C1Cc2cc(Cl)cc(-c3ccnc4c3CCC4)c2O1)N1CCNCC1 ; O=C(C1Cc2cc(Cl)cc(-c3ccnc4cc(C(O)CCl)sc34)c2O1)N1CCNCC1 ; N#CCC(O)c1cc2nccc(-c3cc(Cl)cc4c3OC(C(=O)N3CCNCC3)C4)c2s1 ; O=C(C1Cc2cc(Cl)cc(-c3ccnc4cc(C5(O)COC5)sc34)c2O1)N1CCNCC1]

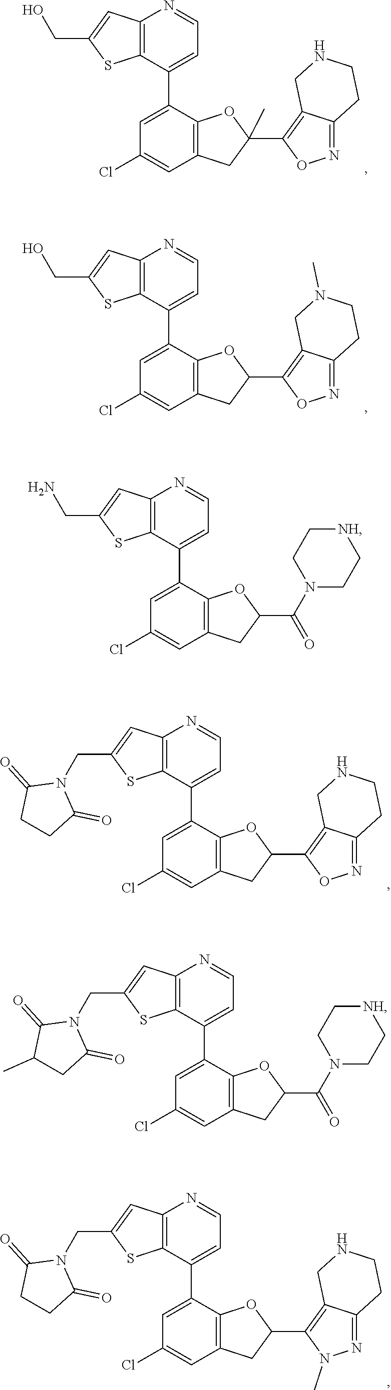 [SMILES: CC1(c2onc3c2CNCC3)Cc2cc(Cl)cc(-c3ccnc4cc(CO)sc34)c2O1 ; CN1CCc2noc(C3Cc4cc(Cl)cc(-c5ccnc6cc(CO)sc56)c4O3)c2C1 ; NCc1cc2nccc(-c3cc(Cl)cc4c3OC(C(=O)N3CCNCC3)C4)c2s1 ; O=C1CCC(=O)N1Cc1cc2nccc(-c3cc(Cl)cc4c3OC(c3onc5c3CNCC5)C4)c2s1 ; CC1CC(=O)N(Cc2cc3nccc(-c4cc(Cl)cc5c4OC(C(=O)N4CCNCC4)C5)c3s2)C1=O ; Cn1nc2c(c1C1Cc3cc(Cl)cc(-c4ccnc5cc(CN6C(=O)CCC6=O)sc45)c3O1)CNCC2]

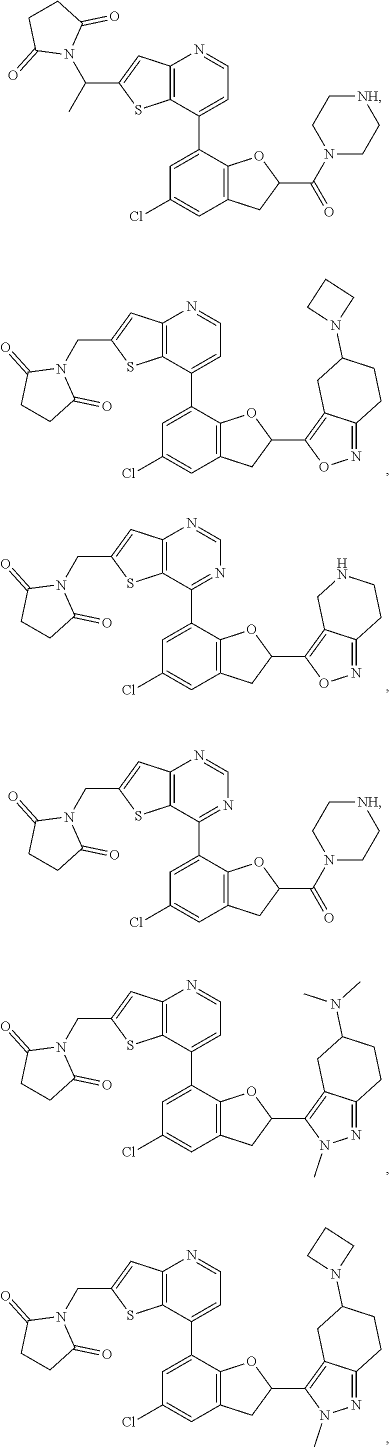 [SMILES: CC(c1cc2nccc(-c3cc(Cl)cc4c3OC(C(=O)N3CCNCC3)C4)c2s1)N1C(=O)CCC1=O ; O=C1CCC(=O)N1Cc1cc2nccc(-c3cc(Cl)cc4c3OC(c3onc5c3CC(N3CCC3)CC5)C4)c2s1 ; O=C1CCC(=O)N1Cc1cc2ncnc(-c3cc(Cl)cc4c3OC(c3onc5c3CNCC5)C4)c2s1 ; O=C(C1Cc2cc(Cl)cc(-c3ncnc4cc(CN5C(=O)CCC5=O)sc34)c2O1)N1CCNCC1 ; CN(C)C1CCc2nn(C)c(C3Cc4cc(Cl)cc(-c5ccnc6cc(CN7C(=O)CCC7=O)sc56)c4O3)c2C1 ; Cn1nc2c(c1C1Cc3cc(Cl)cc(-c4ccnc5cc(CN6C(=O)CCC6=O)sc45)c3O1)CC(N1CCC1)CC2]

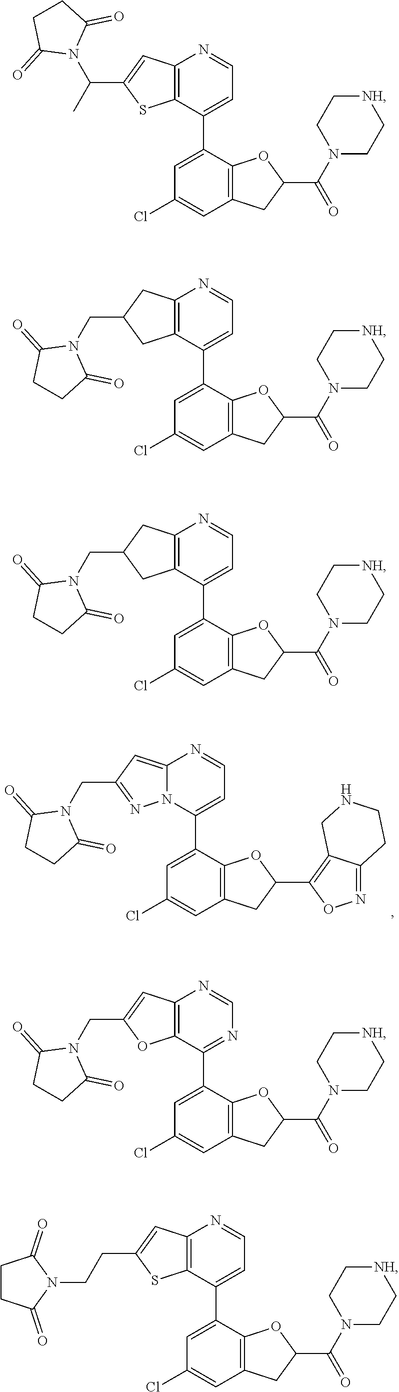 [SMILES: CC(c1cc2nccc(-c3cc(Cl)cc4c3OC(C(=O)N3CCNCC3)C4)c2s1)N1C(=O)CCC1=O ; O=C(C1Cc2cc(Cl)cc(-c3ccnc4c3CC(CN3C(=O)CCC3=O)C4)c2O1)N1CCNCC1 ; O=C(C1Cc2cc(Cl)cc(-c3ccnc4c3CC(CN3C(=O)CCC3=O)C4)c2O1)N1CCNCC1 ; O=C1CCC(=O)N1Cc1cc2nccc(-c3cc(Cl)cc4c3OC(c3onc5c3CNCC5)C4)n2n1 ; O=C(C1Cc2cc(Cl)cc(-c3ncnc4cc(CN5C(=O)CCC5=O)oc34)c2O1)N1CCNCC1 ; O=C(C1Cc2cc(Cl)cc(-c3ccnc4cc(CCN5C(=O)CCC5=O)sc34)c2O1)N1CCNCC1]

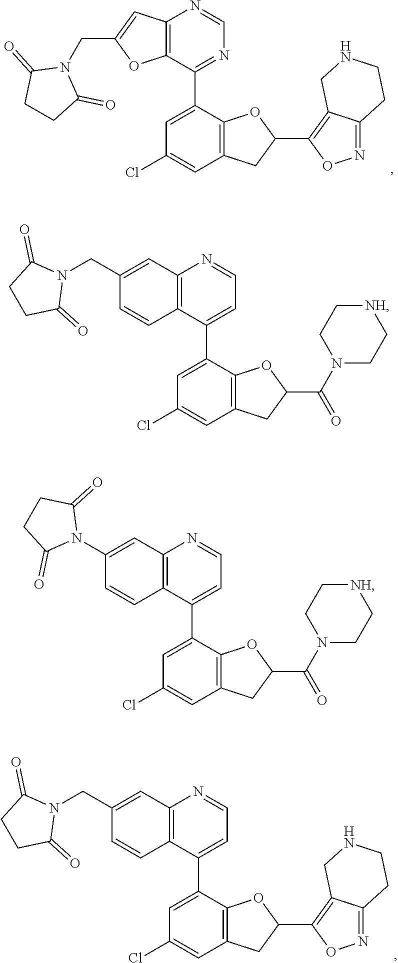 [SMILES: O=C1CCC(=O)N1Cc1cc2ncnc(-c3cc(Cl)cc4c3OC(c3onc5c3CNCC5)C4)c2o1 ; O=C(C1Cc2cc(Cl)cc(-c3ccnc4cc(CN5C(=O)CCC5=O)ccc34)c2O1)N1CCNCC1 ; O=C(C1Cc2cc(Cl)cc(-c3ccnc4cc(N5C(=O)CCC5=O)ccc34)c2O1)N1CCNCC1 ; O=C1CCC(=O)N1Cc1ccc2c(-c3cc(Cl)cc4c3OC(c3onc5c3CNCC5)C4)ccnc2c1]

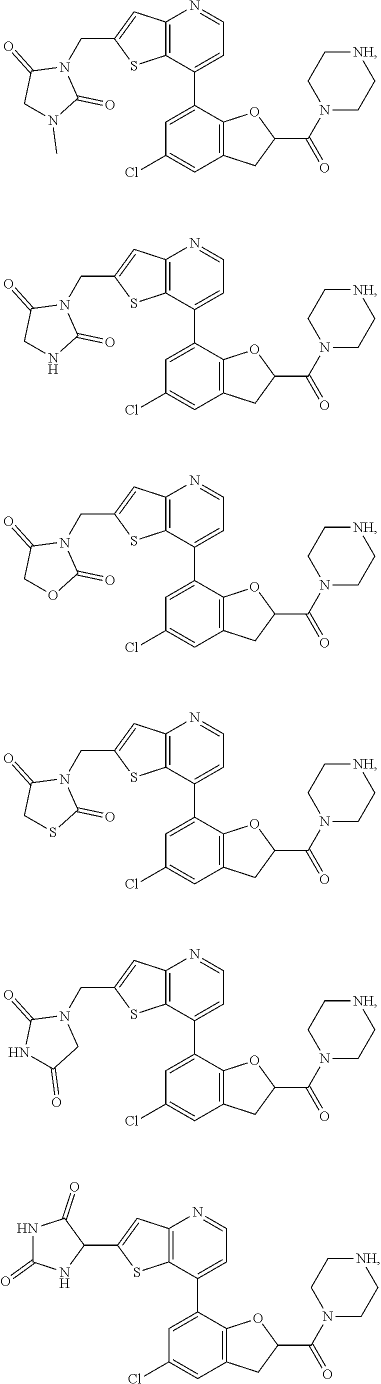 [SMILES: CN1CC(=O)N(Cc2cc3nccc(-c4cc(Cl)cc5c4OC(C(=O)N4CCNCC4)C5)c3s2)C1=O ; O=C(C1Cc2cc(Cl)cc(-c3ccnc4cc(CN5C(=O)CNC5=O)sc34)c2O1)N1CCNCC1 ; O=C(C1Cc2cc(Cl)cc(-c3ccnc4cc(CN5C(=O)COC5=O)sc34)c2O1)N1CCNCC1 ; O=C(C1Cc2cc(Cl)cc(-c3ccnc4cc(CN5C(=O)CSC5=O)sc34)c2O1)N1CCNCC1 ; O=C1CN(Cc2cc3nccc(-c4cc(Cl)cc5c4OC(C(=O)N4CCNCC4)C5)c3s2)C(=O)N1 ; O=C1NC(=O)C(c2cc3nccc(-c4cc(Cl)cc5c4OC(C(=O)N4CCNCC4)C5)c3s2)N1]

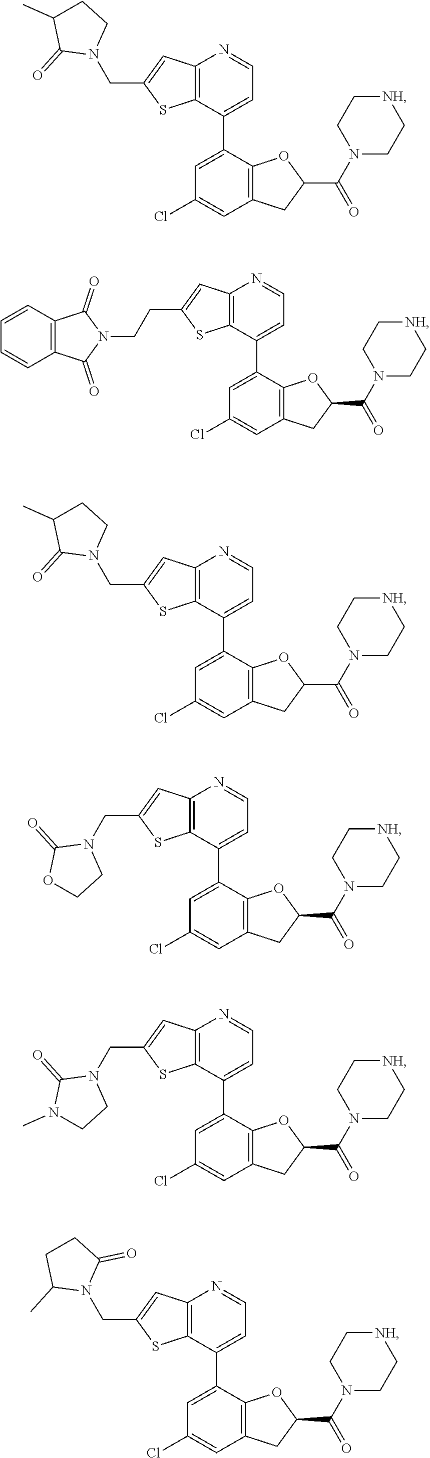 [SMILES: CC1CCN(Cc2cc3nccc(-c4cc(Cl)cc5c4OC(C(=O)N4CCNCC4)C5)c3s2)C1=O ; O=C([C@H]1Cc2cc(Cl)cc(-c3ccnc4cc(CCN5C(=O)c6ccccc6C5=O)sc34)c2O1)N1CCNCC1 ; CC1CCN(Cc2cc3nccc(-c4cc(Cl)cc5c4OC(C(=O)N4CCNCC4)C5)c3s2)C1=O ; O=C1OCCN1Cc1cc2nccc(-c3cc(Cl)cc4c3O[C@@H](C(=O)N3CCNCC3)C4)c2s1 ; CN1CCN(Cc2cc3nccc(-c4cc(Cl)cc5c4O[C@@H](C(=O)N4CCNCC4)C5)c3s2)C1=O ; CC1CCC(=O)N1Cc1cc2nccc(-c3cc(Cl)cc4c3O[C@@H](C(=O)N3CCNCC3)C4)c2s1]

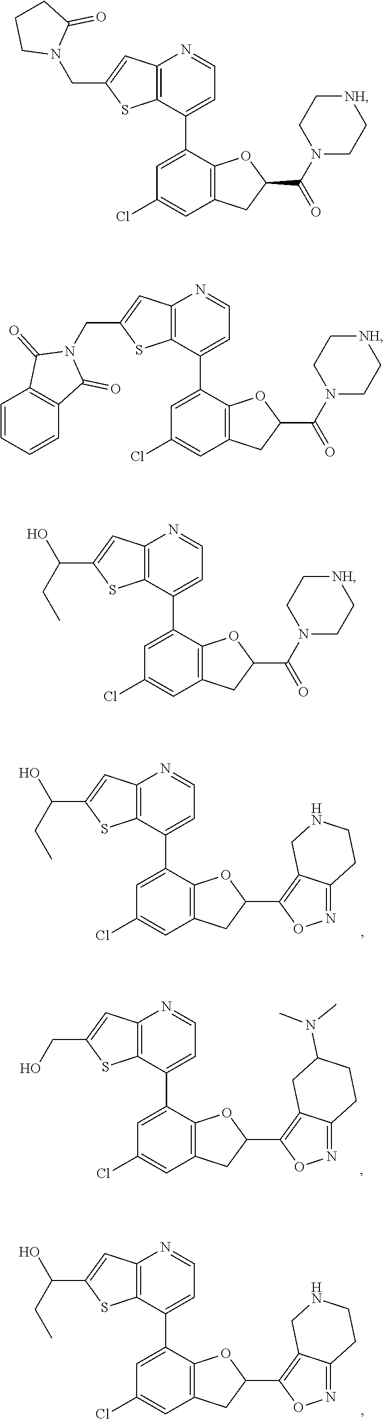 [SMILES: O=C1CCCN1Cc1cc2nccc(-c3cc(Cl)cc4c3O[C@@H](C(=O)N3CCNCC3)C4)c2s1 ; O=C(C1Cc2cc(Cl)cc(-c3ccnc4cc(CN5C(=O)c6ccccc6C5=O)sc34)c2O1)N1CCNCC1 ; CCC(O)c1cc2nccc(-c3cc(Cl)cc4c3OC(C(=O)N3CCNCC3)C4)c2s1 ; CCC(O)c1cc2nccc(-c3cc(Cl)cc4c3OC(c3onc5c3CNCC5)C4)c2s1 ; CN(C)C1CCc2noc(C3Cc4cc(Cl)cc(-c5ccnc6cc(CO)sc56)c4O3)c2C1 ; CCC(O)c1cc2nccc(-c3cc(Cl)cc4c3OC(c3onc5c3CNCC5)C4)c2s1]

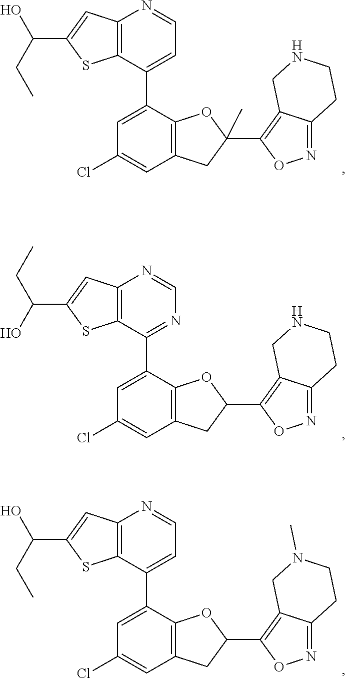 [SMILES: CCC(O)c1cc2nccc(-c3cc(Cl)cc4c3OC(C)(c3onc5c3CNCC5)C4)c2s1 ; CCC(O)c1cc2ncnc(-c3cc(Cl)cc4c3OC(c3onc5c3CNCC5)C4)c2s1 ; CCC(O)c1cc2nccc(-c3cc(Cl)cc4c3OC(c3onc5c3CN(C)CC5)C4)c2s1]

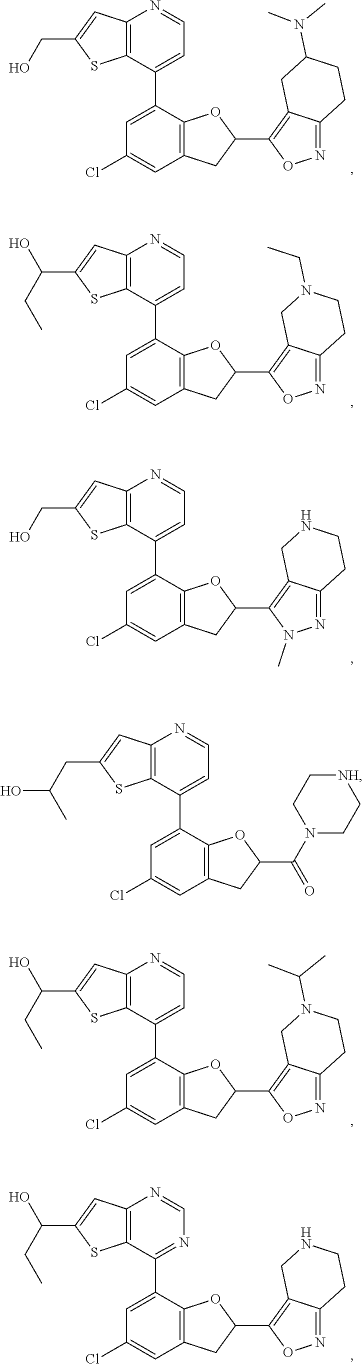 [SMILES: CN(C)C1CCc2noc(C3Cc4cc(Cl)cc(-c5ccnc6cc(CO)sc56)c4O3)c2C1 ; CCC(O)c1cc2nccc(-c3cc(Cl)cc4c3OC(c3onc5c3CN(CC)CC5)C4)c2s1 ; Cn1nc2c(c1C1Cc3cc(Cl)cc(-c4ccnc5cc(CO)sc45)c3O1)CNCC2 ; CC(O)Cc1cc2nccc(-c3cc(Cl)cc4c3OC(C(=O)N3CCNCC3)C4)c2s1 ; CCC(O)c1cc2nccc(-c3cc(Cl)cc4c3OC(c3onc5c3CN(C(C)C)CC5)C4)c2s1 ; CCC(O)c1cc2ncnc(-c3cc(Cl)cc4c3OC(c3onc5c3CNCC5)C4)c2s1]

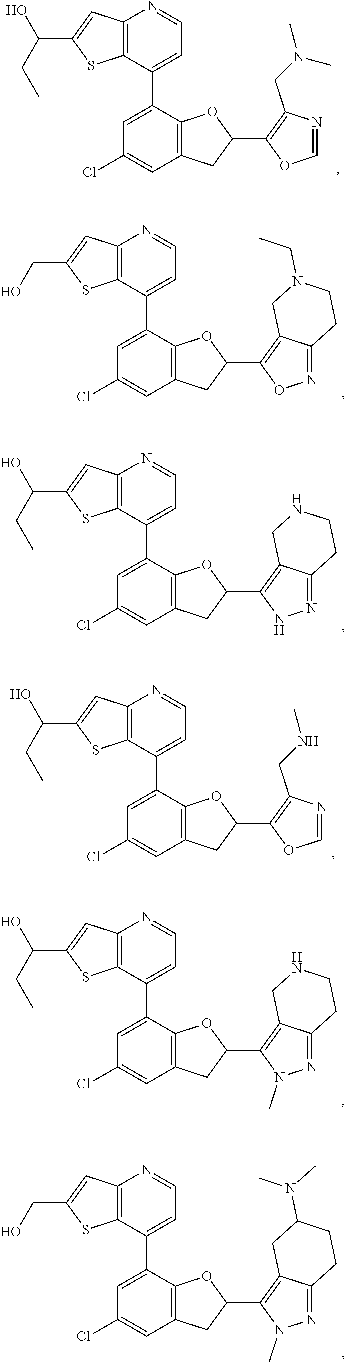 [SMILES: CCC(O)c1cc2nccc(-c3cc(Cl)cc4c3OC(c3ocnc3CN(C)C)C4)c2s1 ; CCN1CCc2noc(C3Cc4cc(Cl)cc(-c5ccnc6cc(CO)sc56)c4O3)c2C1 ; CCC(O)c1cc2nccc(-c3cc(Cl)cc4c3OC(c3[nH]nc5c3CNCC5)C4)c2s1 ; CCC(O)c1cc2nccc(-c3cc(Cl)cc4c3OC(c3ocnc3CNC)C4)c2s1 ; CCC(O)c1cc2nccc(-c3cc(Cl)cc4c3OC(c3c5c(nn3C)CCNC5)C4)c2s1 ; CN(C)C1CCc2nn(C)c(C3Cc4cc(Cl)cc(-c5ccnc6cc(CO)sc56)c4O3)c2C1]

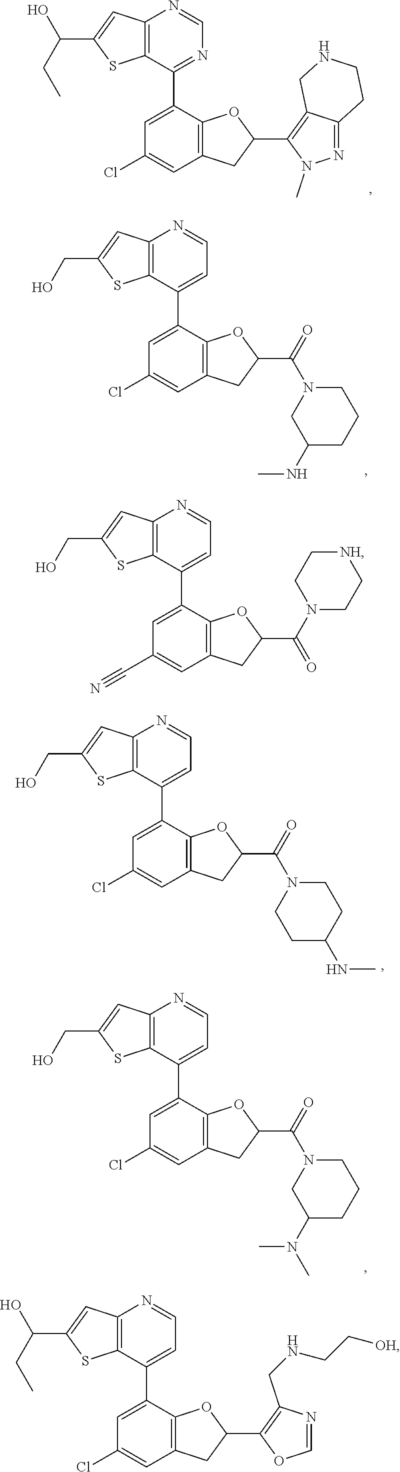

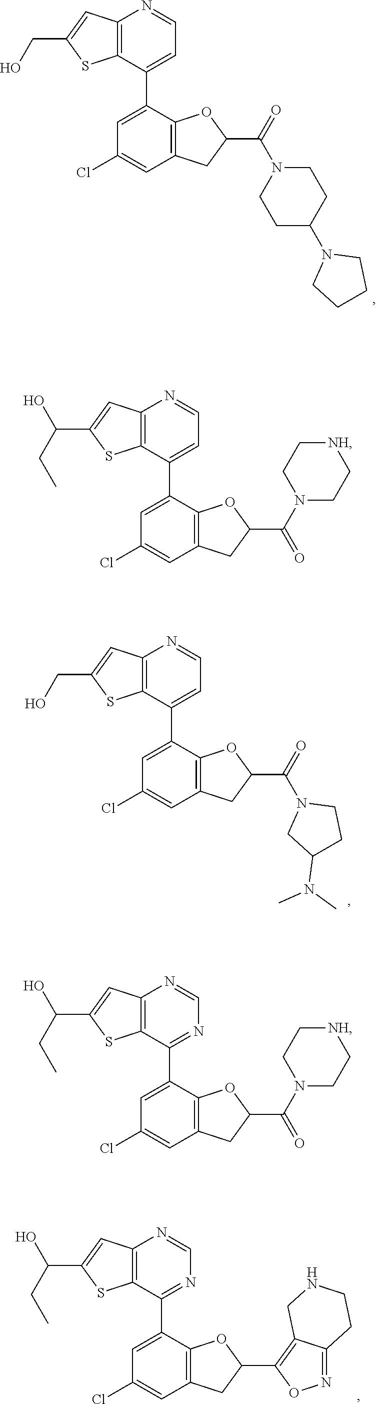 [SMILES: O=C(C1Cc2cc(Cl)cc(-c3ccnc4cc(CO)sc34)c2O1)N1CCC(N2CCCC2)CC1 ; CCC(O)c1cc2nccc(-c3cc(Cl)cc4c3OC(C(=O)N3CCNCC3)C4)c2s1 ; CN(C)C1CCN(C(=O)C2Cc3cc(Cl)cc(-c4ccnc5cc(CO)sc45)c3O2)C1 ; CCC(O)c1cc2ncnc(-c3cc(Cl)cc4c3OC(C(=O)N3CCNCC3)C4)c2s1 ; CCC(O)c1cc2ncnc(-c3cc(Cl)cc4c3OC(c3onc5c3CNCC5)C4)c2s1]

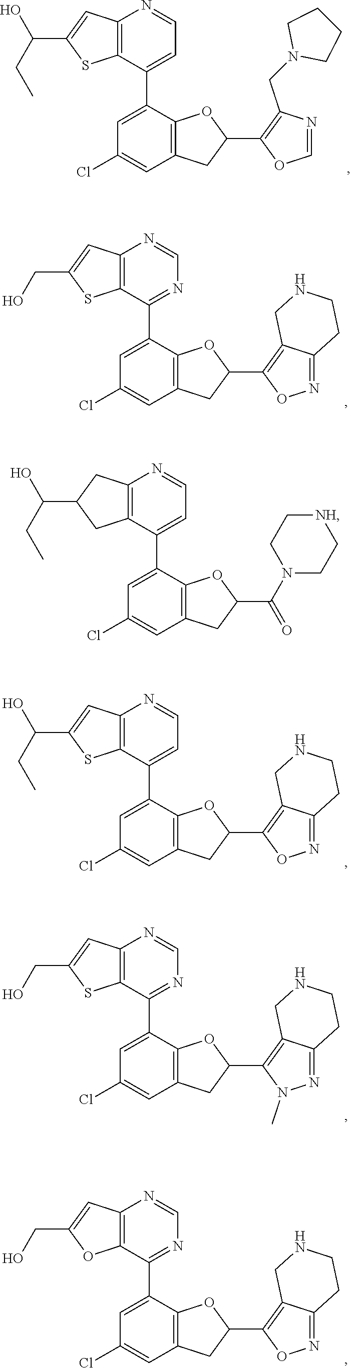 [SMILES: CCC(O)c1cc2nccc(-c3cc(Cl)cc4c3OC(c3ocnc3CN3CCCC3)C4)c2s1 ; OCc1cc2ncnc(-c3cc(Cl)cc4c3OC(c3onc5c3CNCC5)C4)c2s1 ; CCC(O)C1Cc2nccc(-c3cc(Cl)cc4c3OC(C(=O)N3CCNCC3)C4)c2C1 ; CCC(O)c1cc2nccc(-c3cc(Cl)cc4c3OC(c3onc5c3CNCC5)C4)c2s1 ; Cn1nc2c(c1C1Cc3cc(Cl)cc(-c4ncnc5cc(CO)sc45)c3O1)CNCC2 ; OCc1cc2ncnc(-c3cc(Cl)cc4c3OC(c3onc5c3CNCC5)C4)c2o1]

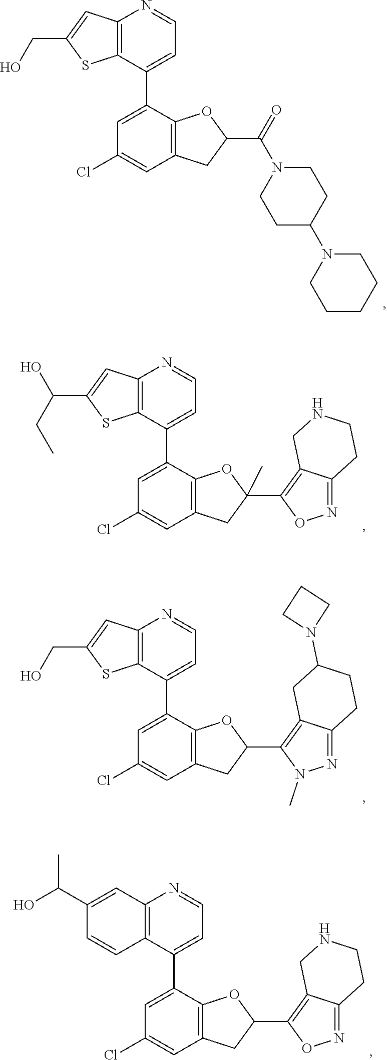 [SMILES: O=C(C1Cc2cc(Cl)cc(-c3ccnc4cc(CO)sc34)c2O1)N1CCC(N2CCCCC2)CC1 ; CCC(O)c1cc2nccc(-c3cc(Cl)cc4c3OC(C)(c3onc5c3CNCC5)C4)c2s1 ; Cn1nc2c(c1C1Cc3cc(Cl)cc(-c4ccnc5cc(CO)sc45)c3O1)CC(N1CCC1)CC2 ; CC(O)c1ccc2c(-c3cc(Cl)cc4c3OC(c3onc5c3CNCC5)C4)ccnc2c1]

In embodiments, the compound has the formula:

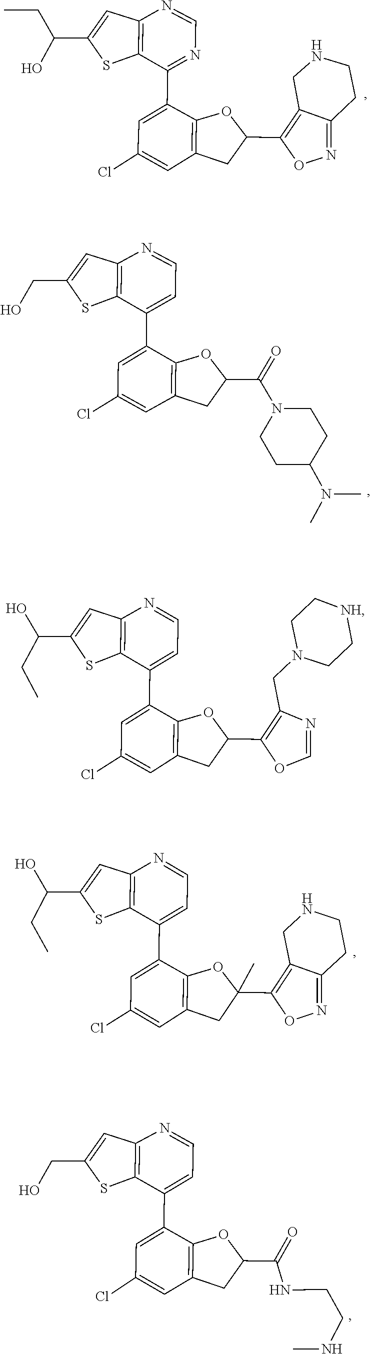 [SMILES: CCC(O)c1cc2ncnc(-c3cc(Cl)cc4c3OC(c3onc5c3CNCC5)C4)c2s1 ; CN(C)C1CCN(C(=O)C2Cc3cc(Cl)cc(-c4ccnc5cc(CO)sc45)c3O2)CC1 ; CCC(O)c1cc2nccc(-c3cc(Cl)cc4c3OC(c3ocnc3CN3CCNCC3)C4)c2s1 ; CCC(O)c1cc2nccc(-c3cc(Cl)cc4c3OC(C)(c3onc5c3CNCC5)C4)c2s1 ; CNCCNC(=O)C1Cc2cc(Cl)cc(-c3ccnc4cc(CO)sc34)c2O1]

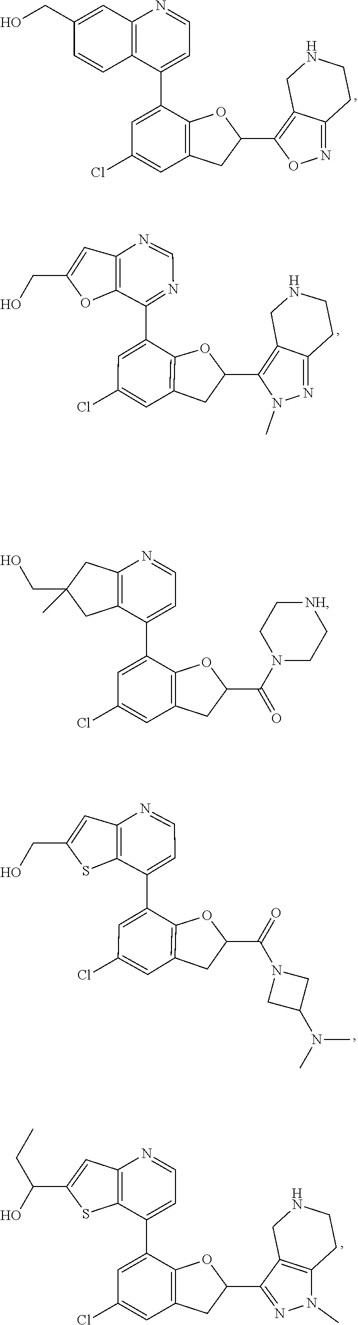 [SMILES: OCc1ccc2c(-c3cc(Cl)cc4c3OC(c3onc5c3CNCC5)C4)ccnc2c1 ; Cn1nc2c(c1C1Cc3cc(Cl)cc(-c4ncnc5cc(CO)oc45)c3O1)CNCC2 ; CC1(CO)Cc2nccc(-c3cc(Cl)cc4c3OC(C(=O)N3CCNCC3)C4)c2C1 ; CN(C)C1CN(C(=O)C2Cc3cc(Cl)cc(-c4ccnc5cc(CO)sc45)c3O2)C1 ; CCC(O)c1cc2nccc(-c3cc(Cl)cc4c3OC(c3nn(C)c5c3CNCC5)C4)c2s1]

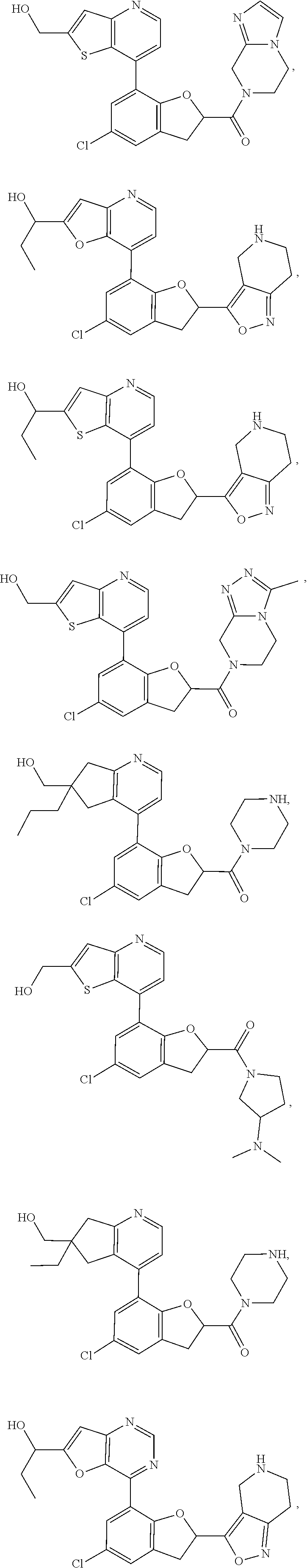

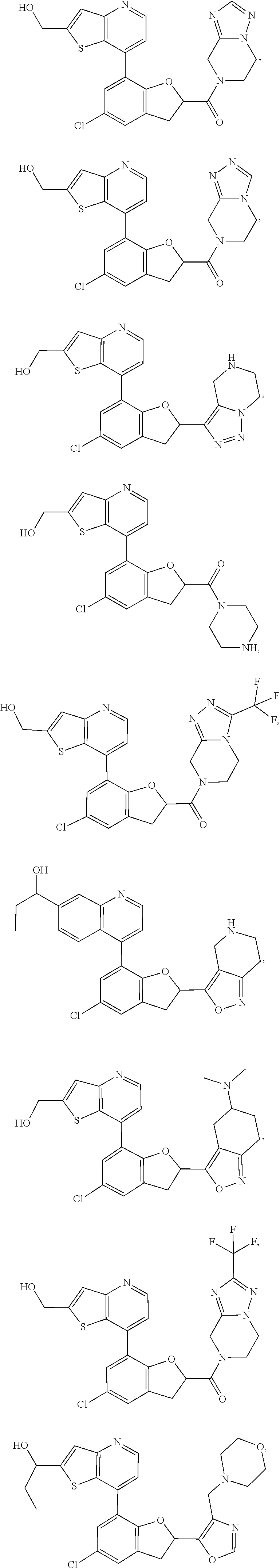

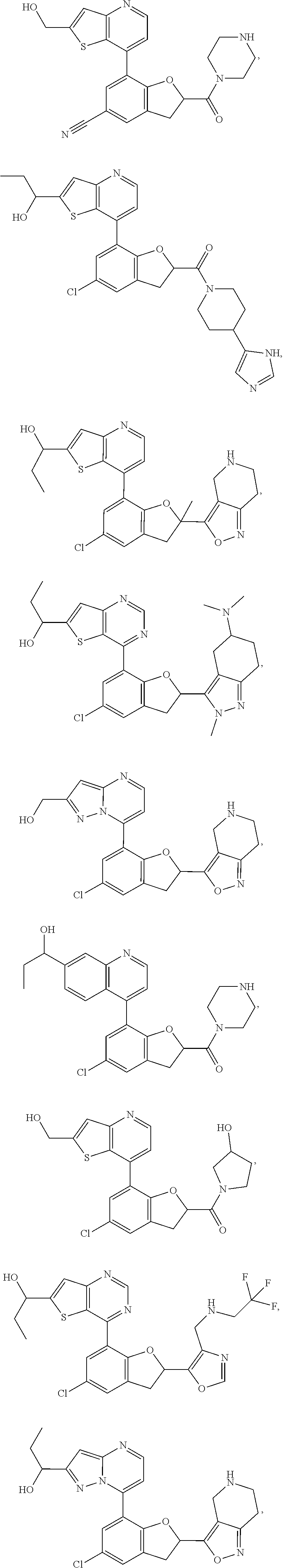

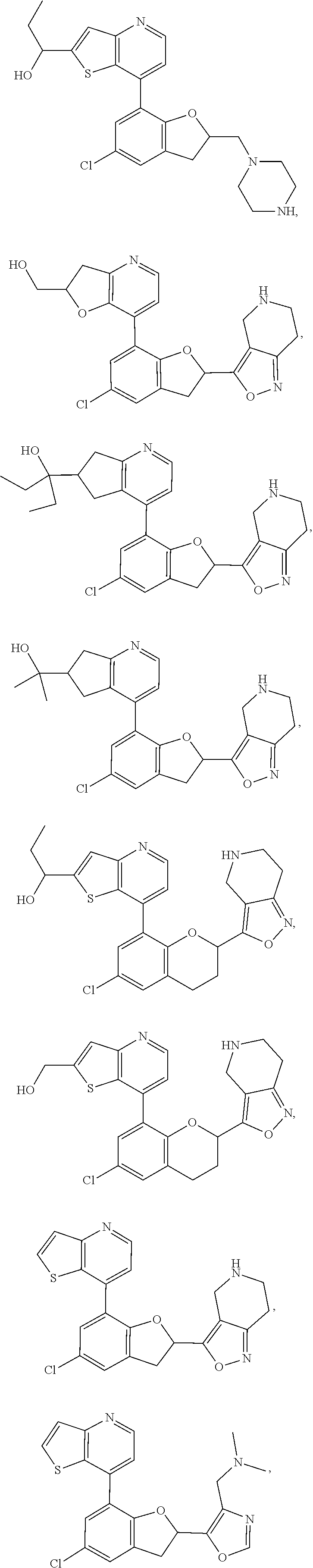

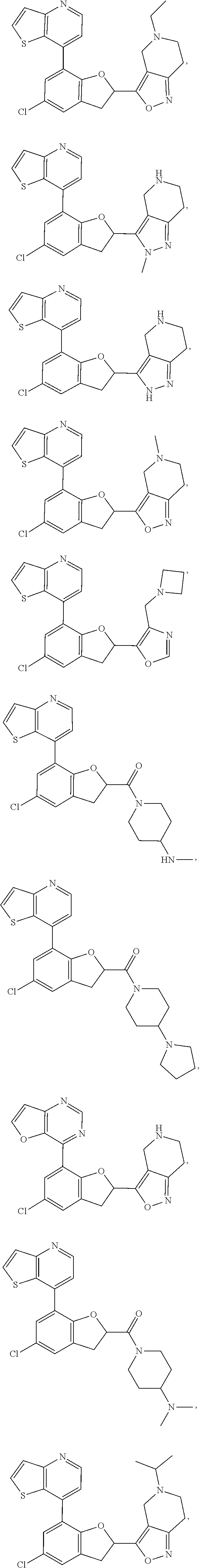

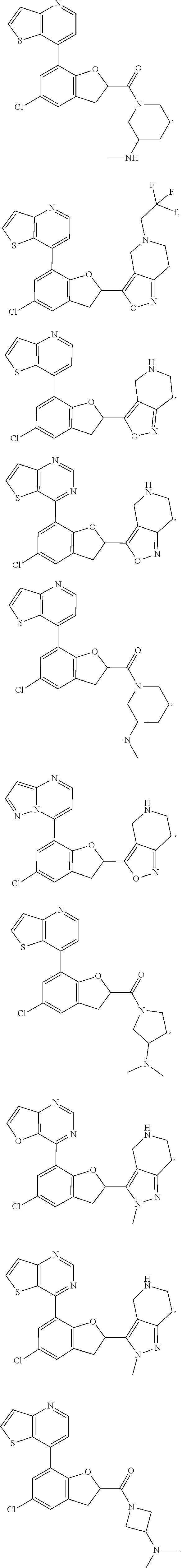

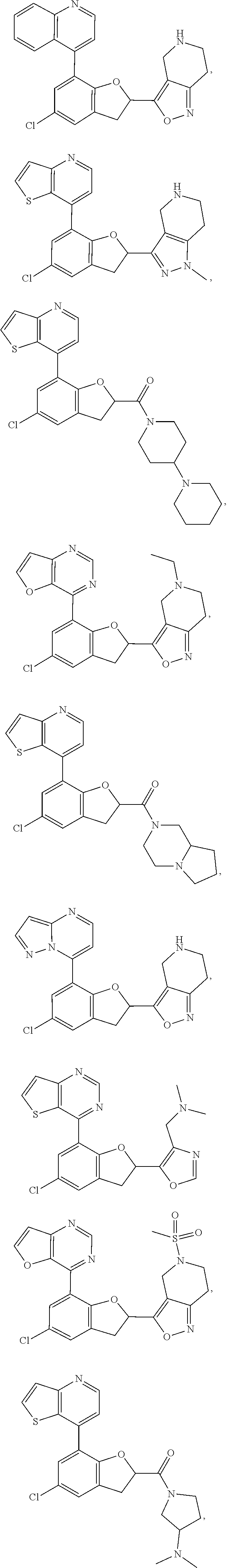

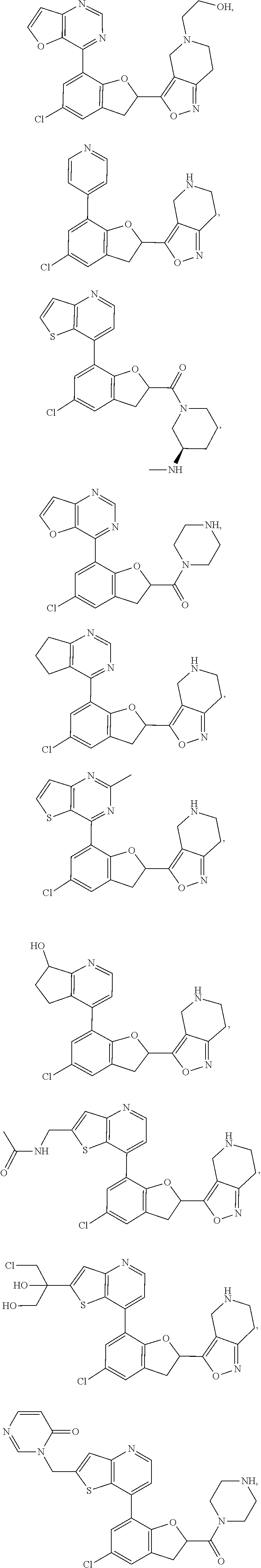

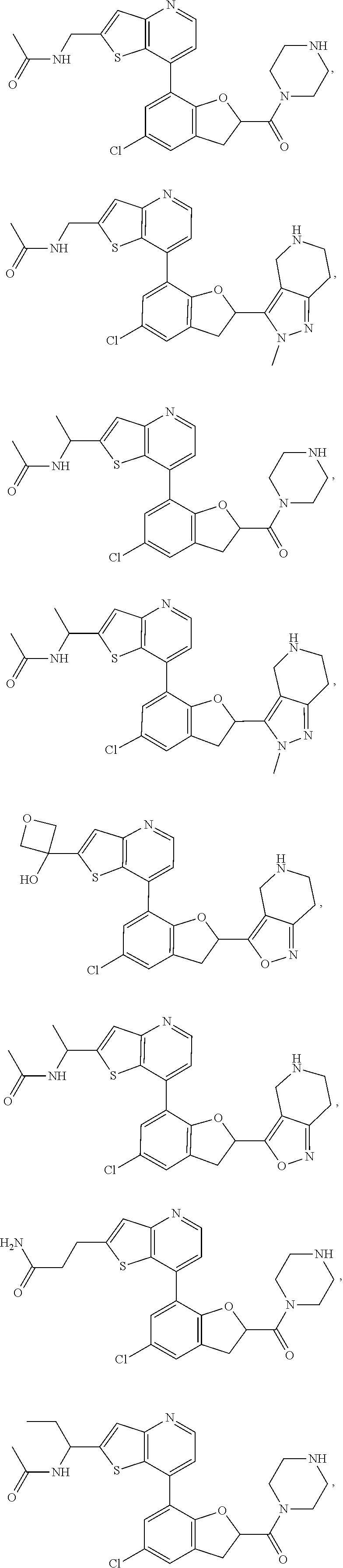

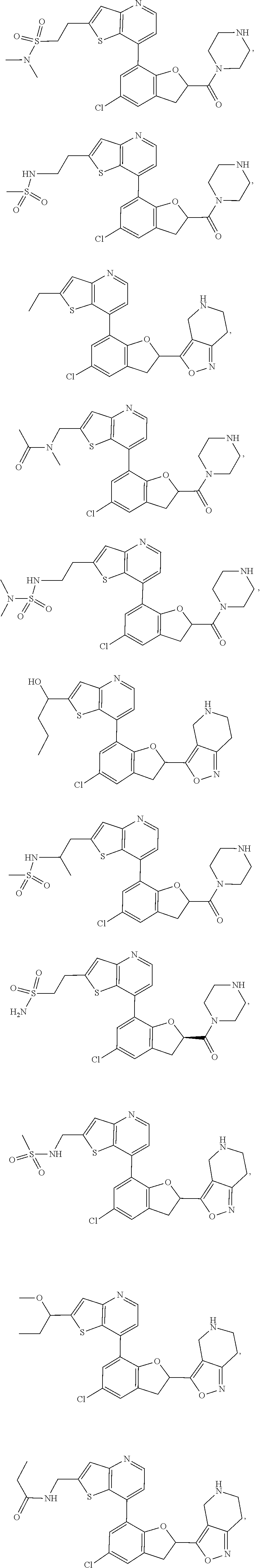

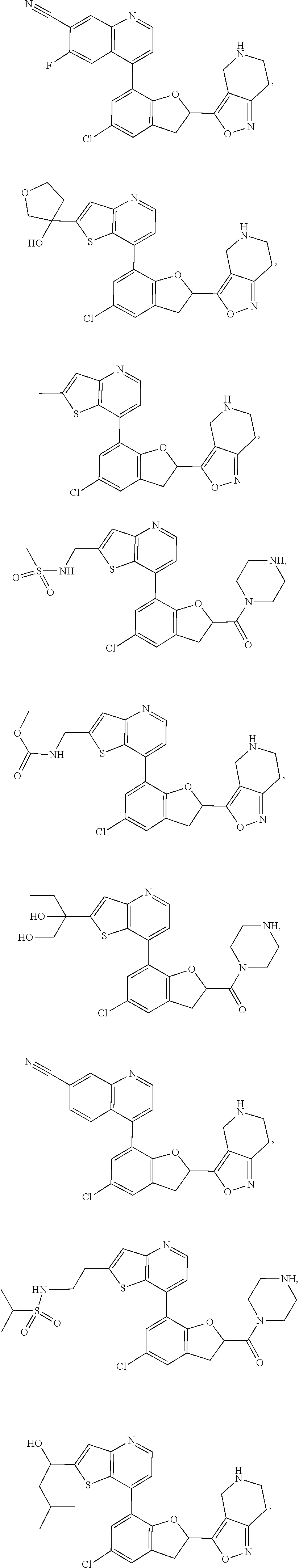

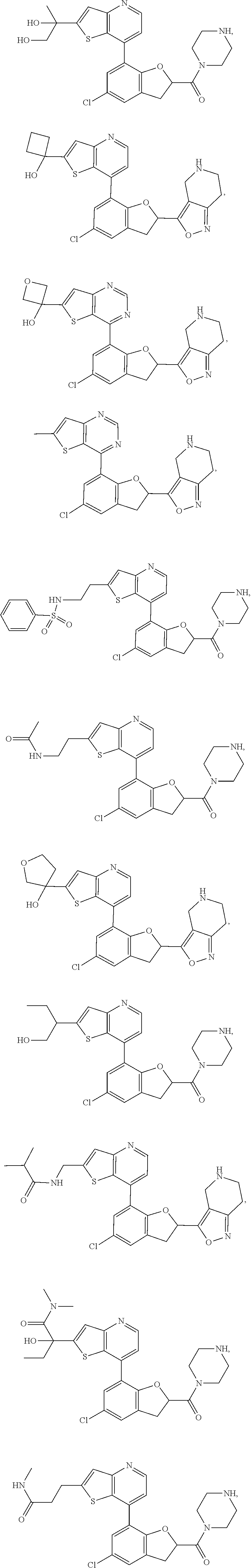

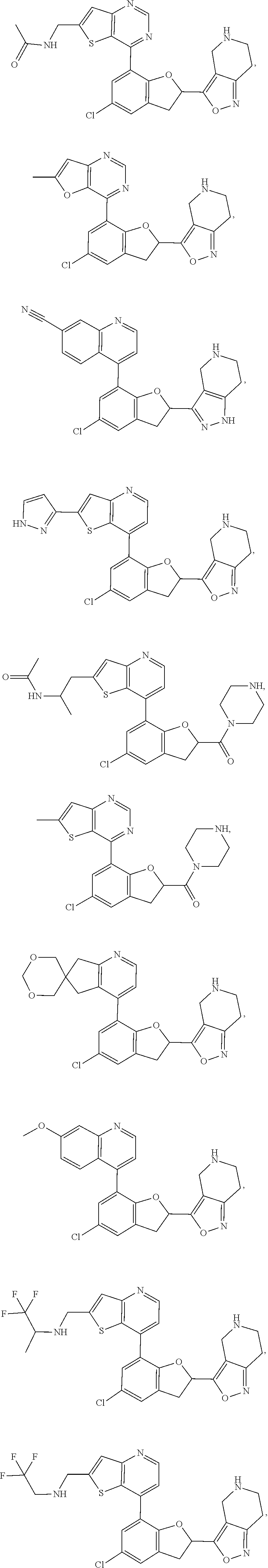

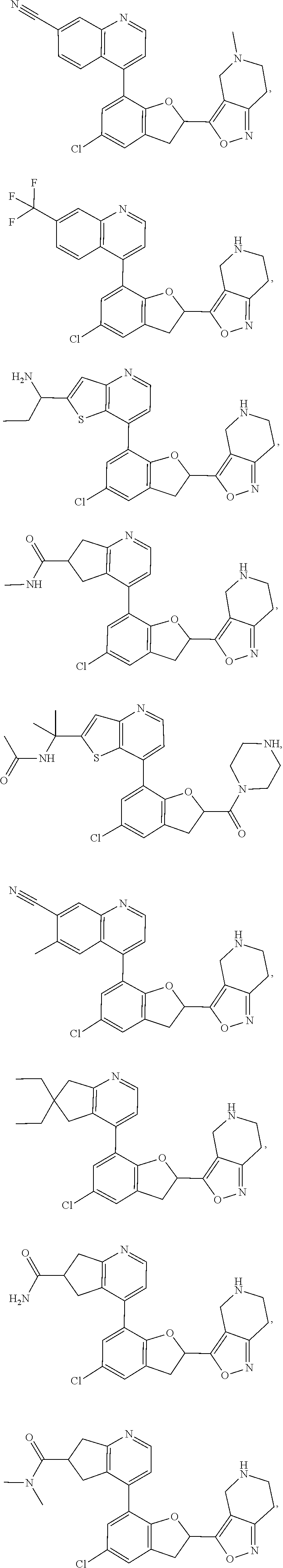

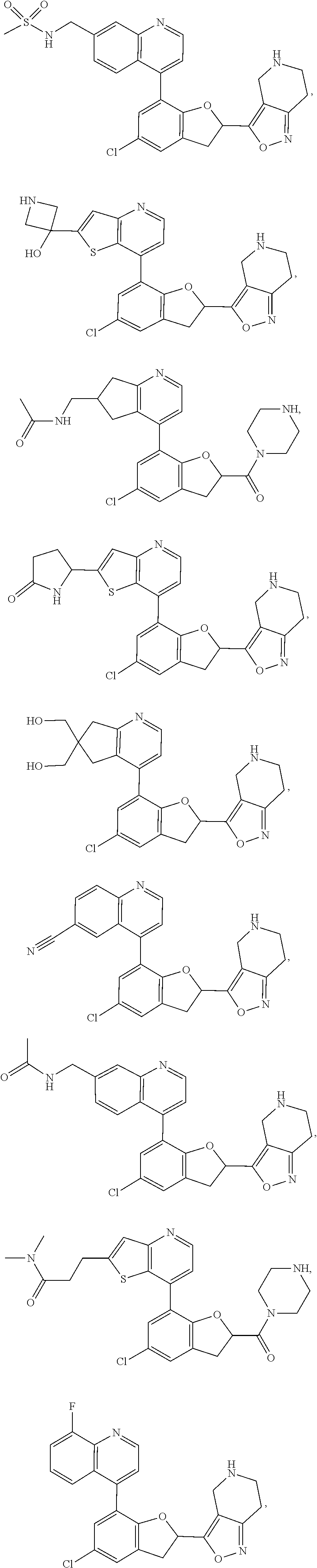

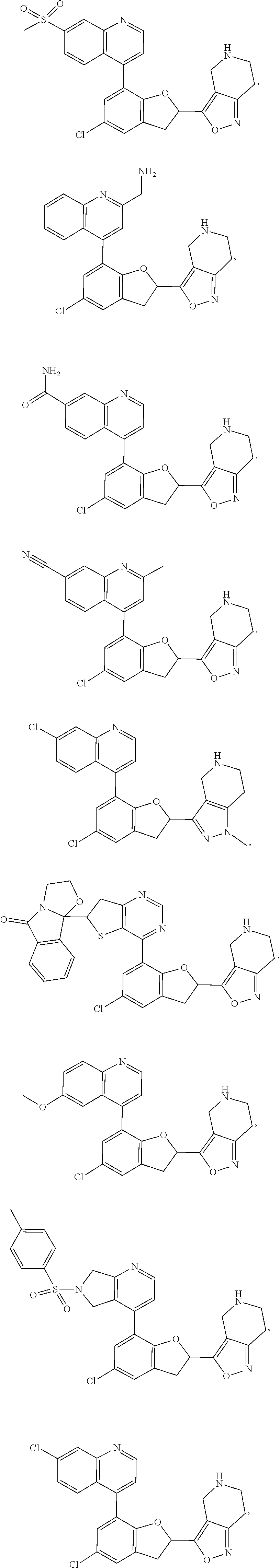

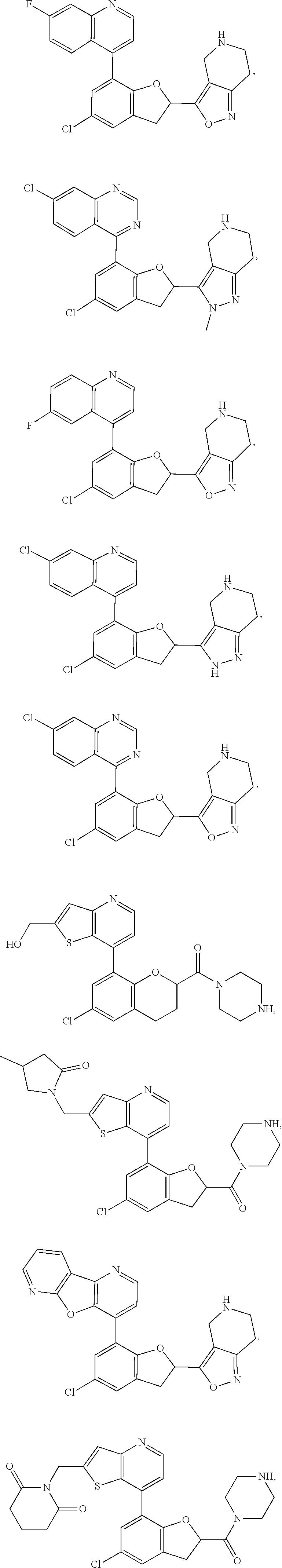

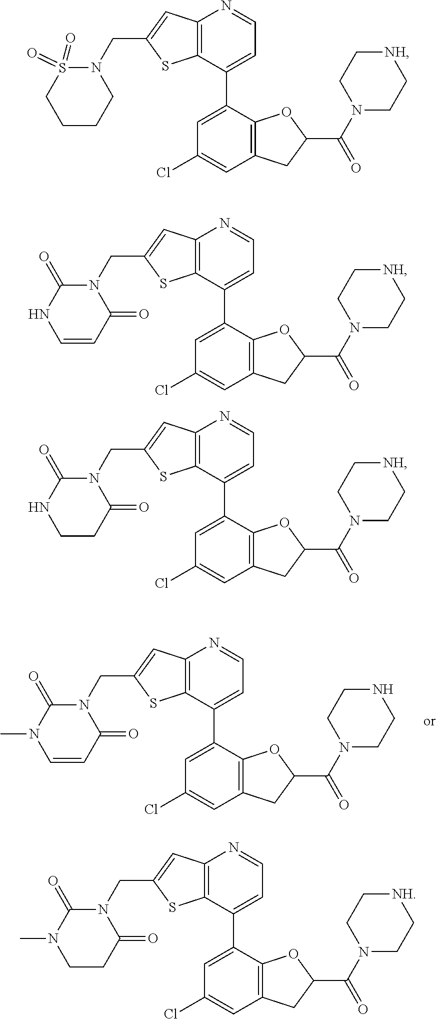

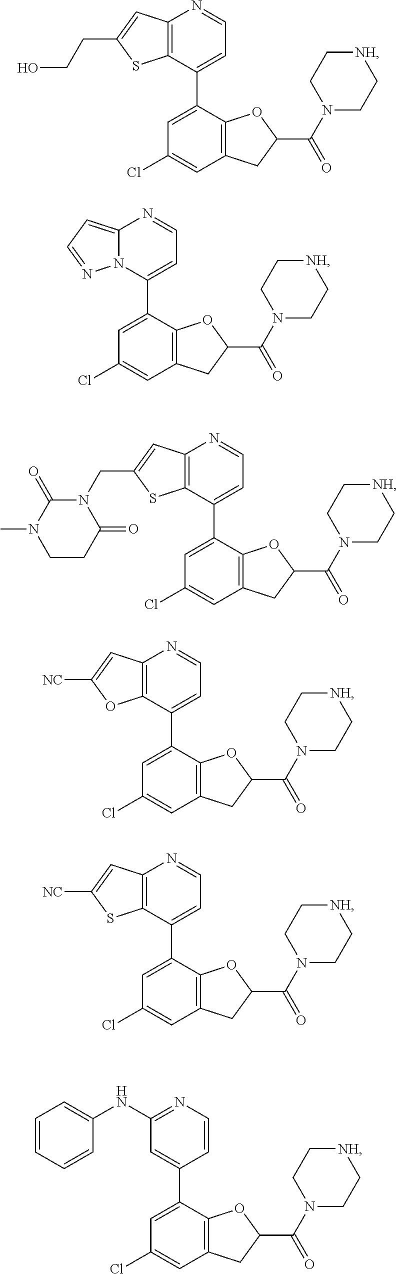

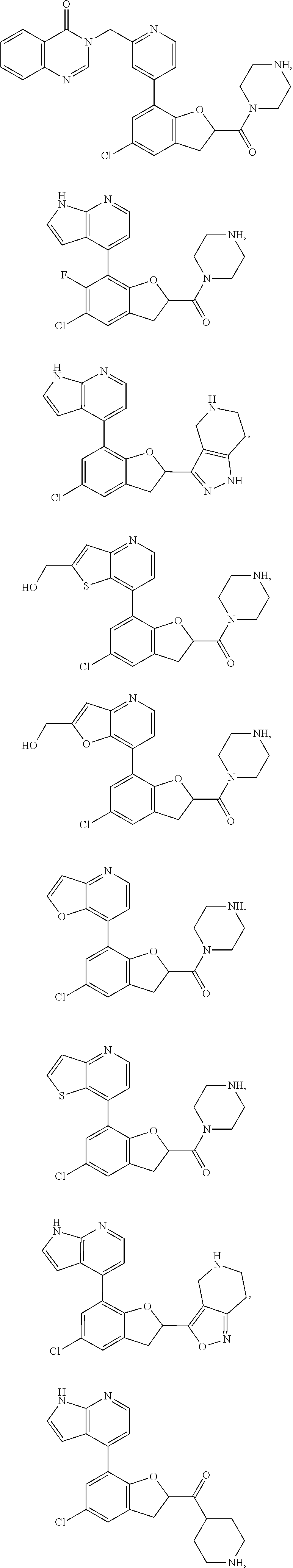

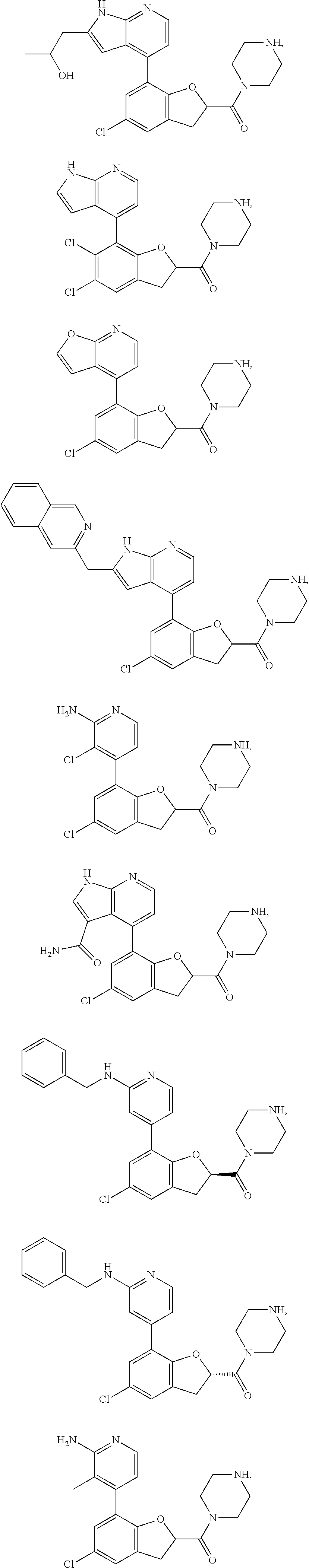

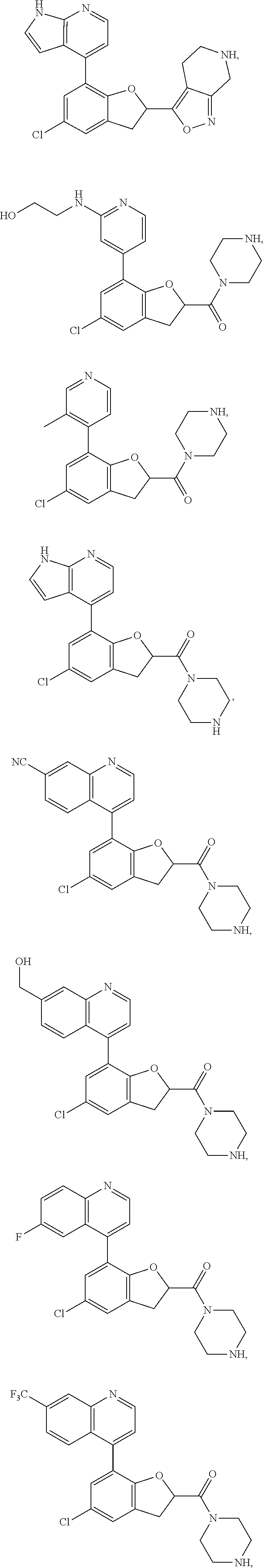

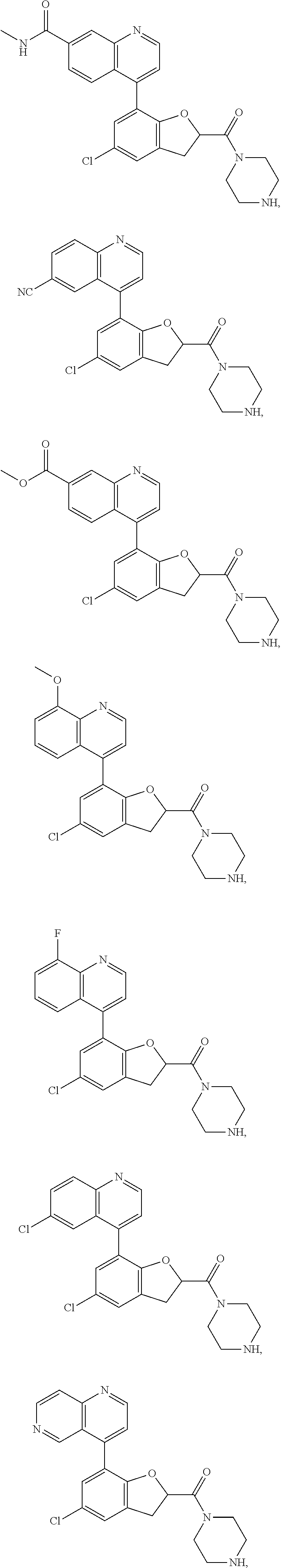

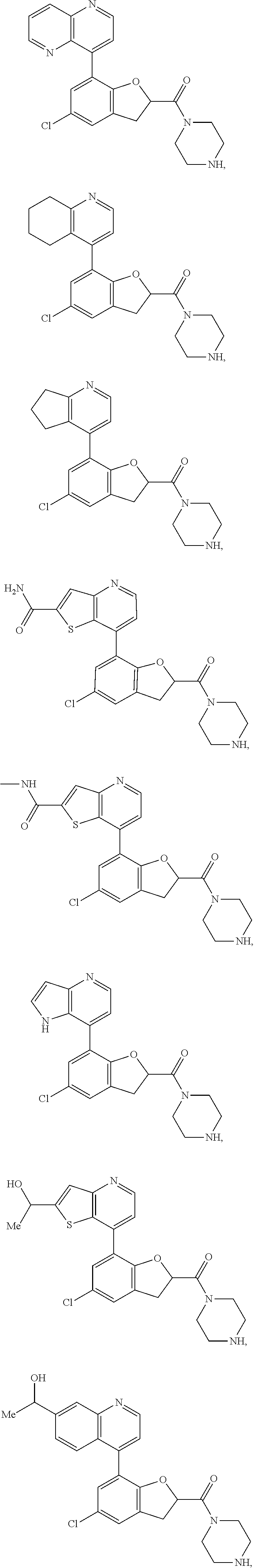

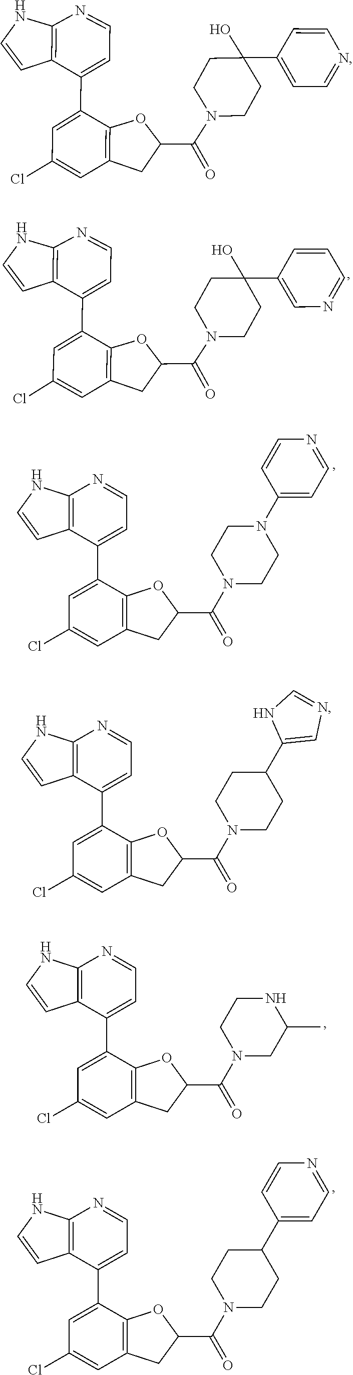 [SMILES: O=C(C1Cc2cc(Cl)cc(-c3ccnc4[nH]ccc34)c2O1)N1CCC(O)(c2ccncc2)CC1 ; O=C(C1Cc2cc(Cl)cc(-c3ccnc4[nH]ccc34)c2O1)N1CCC(O)(c2cccnc2)CC1 ; O=C(C1Cc2cc(Cl)cc(-c3ccnc4[nH]ccc34)c2O1)N1CCN(c2ccncc2)CC1 ; O=C(C1Cc2cc(Cl)cc(-c3ccnc4[nH]ccc34)c2O1)N1CCC(c2cnc[nH]2)CC1 ; CC1CN(C(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(c2ccncc2)CC1]

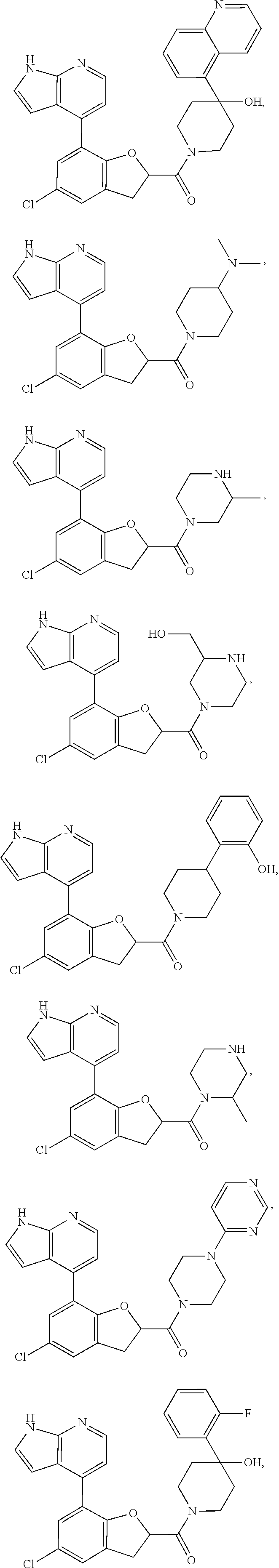

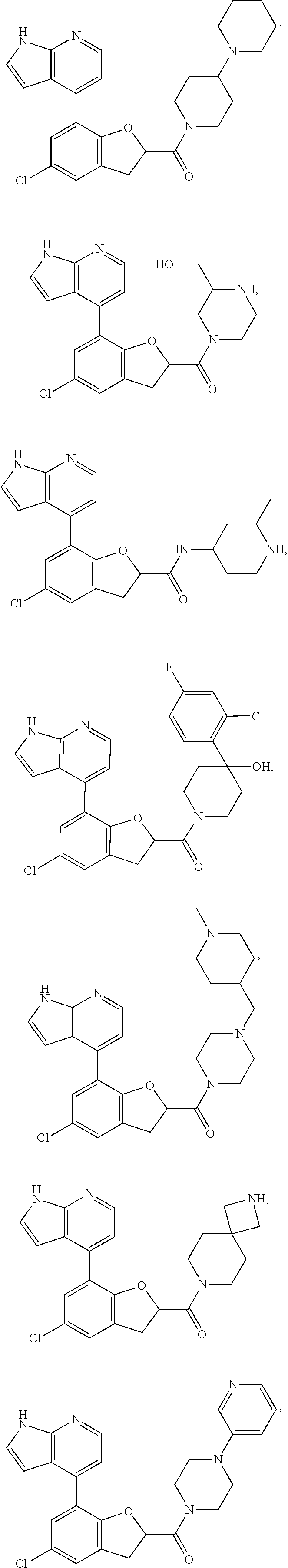

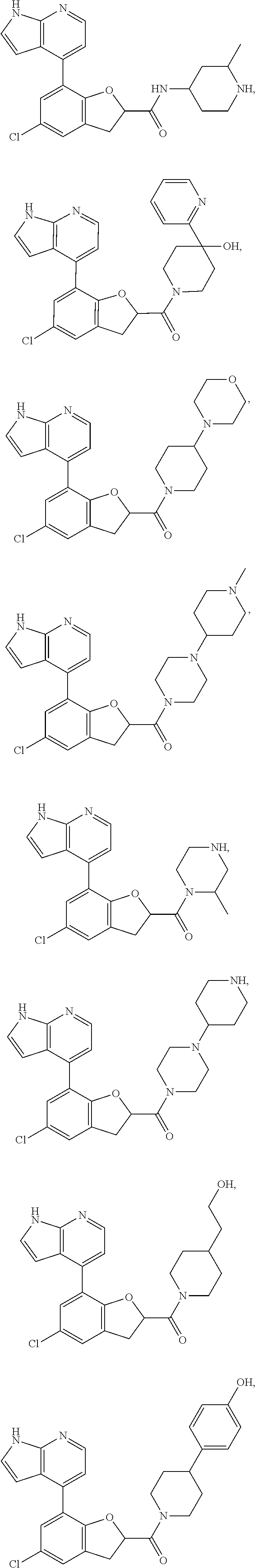

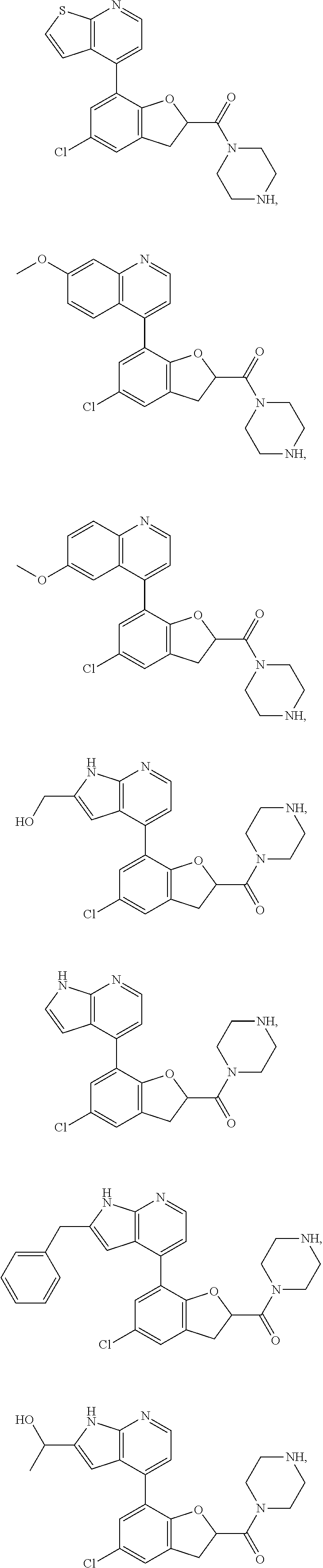

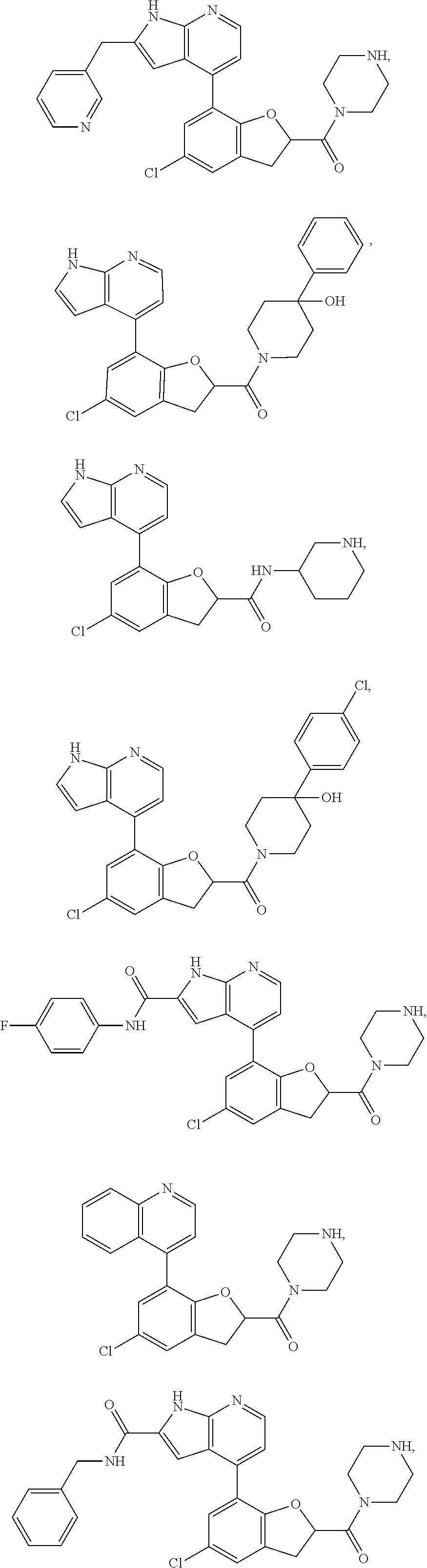

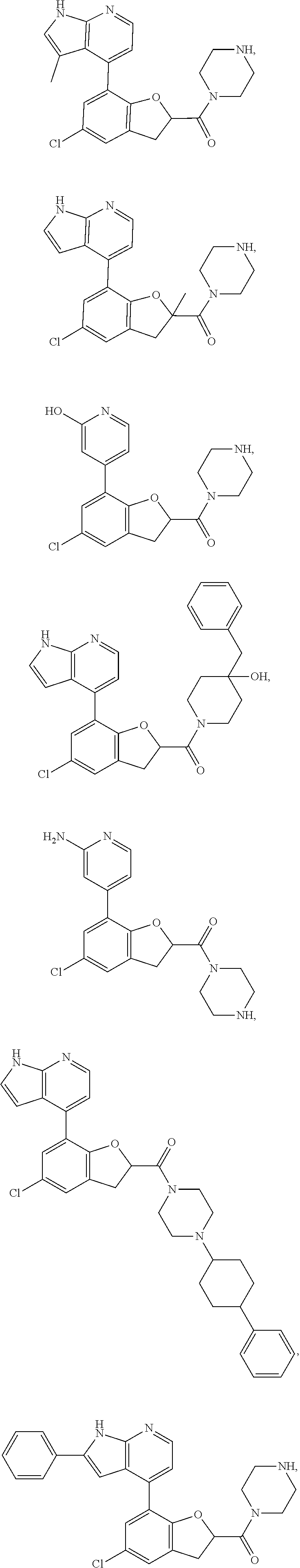

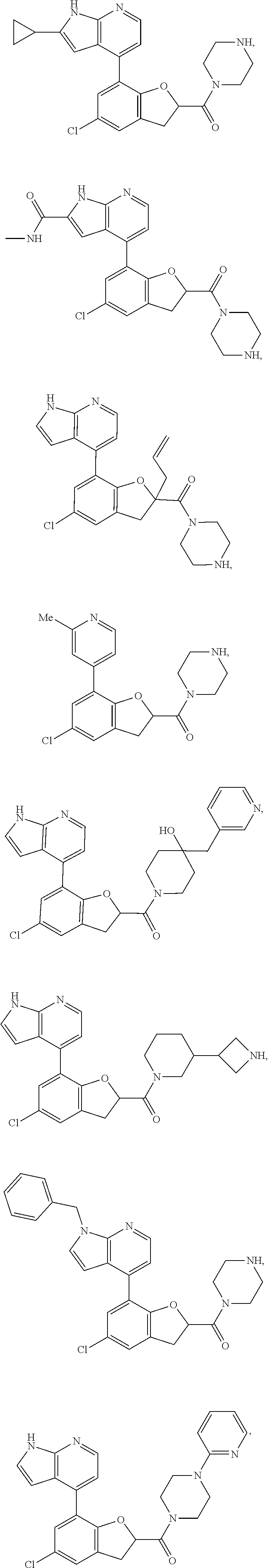

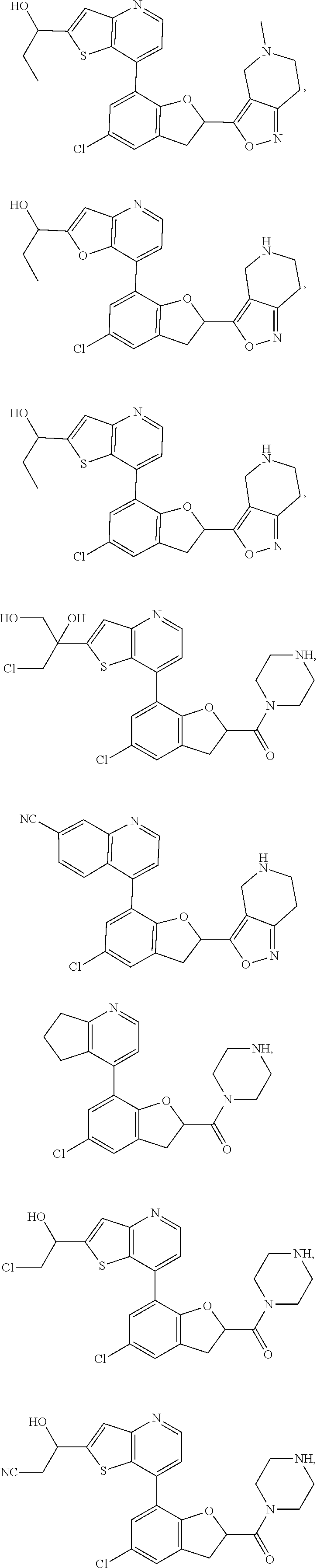

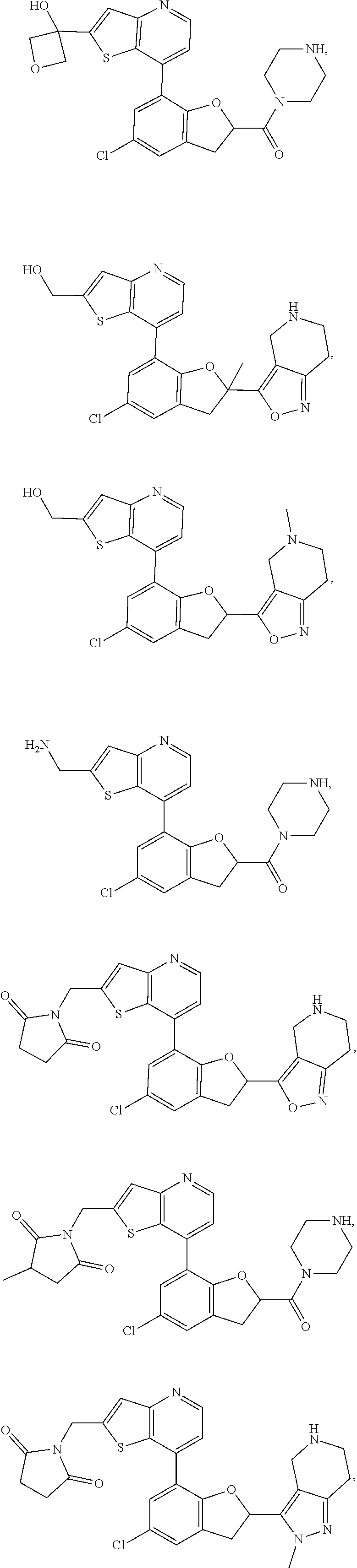

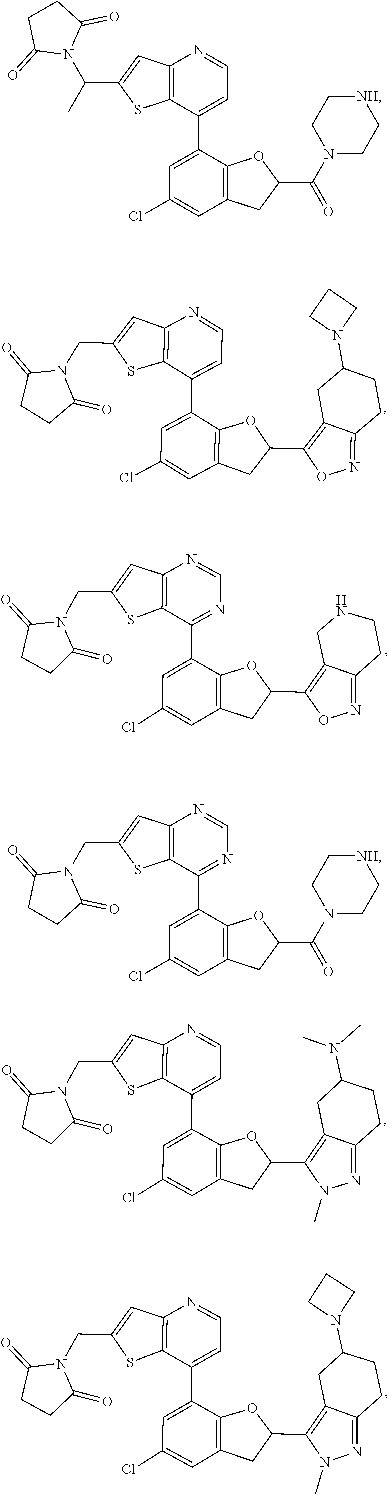 [SMILES: CC(c1cc2nccc(-c3cc(Cl)cc4c3OC(C(=O)N3CCNCC3)C4)c2s1)N1C(=O)CCC1=O ; O=C1CCC(=O)N1Cc1cc2nccc(-c3cc(Cl)cc4c3OC(c3onc5c3CC(N3CCC3)CC5)C4)c2s1 ; O=C1CCC(=O)N1Cc1cc2ncnc(-c3cc(Cl)cc4c3OC(c3onc5c3CNCC5)C4)c2s1 ; O=C(C1Cc2cc(Cl)cc(-c3ncnc4cc(CN5C(=O)CCC5=O)sc34)c2O1)N1CCNCC1 ; CN(C)C1CCc2nn(C)c(C3Cc4cc(Cl)cc(-c5ccnc6cc(CN7C(=O)CCC7=O)sc56)c4O3)c2C1 ; Cn1nc2c(c1C1Cc3cc(Cl)cc(-c4ccnc5cc(CN6C(=O)CCC6=O)sc45)c3O1)CC(N1CCC1)CC2]

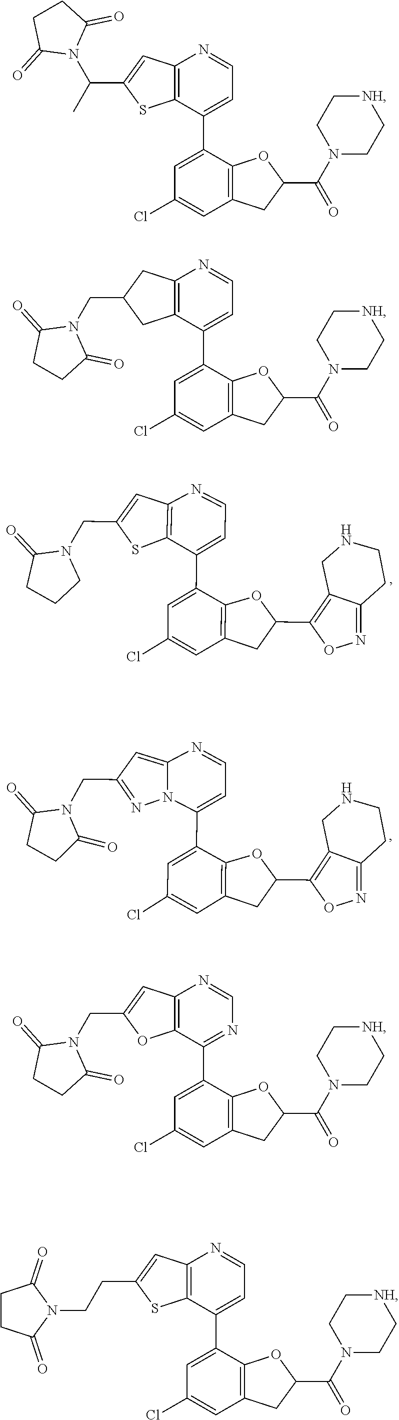 [SMILES: CC(c1cc2nccc(-c3cc(Cl)cc4c3OC(C(=O)N3CCNCC3)C4)c2s1)N1C(=O)CCC1=O ; O=C(C1Cc2cc(Cl)cc(-c3ccnc4c3CC(CN3C(=O)CCC3=O)C4)c2O1)N1CCNCC1 ; O=C1CCCN1Cc1cc2nccc(-c3cc(Cl)cc4c3OC(c3onc5c3CNCC5)C4)c2s1 ; O=C1CCC(=O)N1Cc1cc2nccc(-c3cc(Cl)cc4c3OC(c3onc5c3CNCC5)C4)n2n1 ; O=C(C1Cc2cc(Cl)cc(-c3ncnc4cc(CN5C(=O)CCC5=O)oc34)c2O1)N1CCNCC1 ; O=C(C1Cc2cc(Cl)cc(-c3ccnc4cc(CCN5C(=O)CCC5=O)sc34)c2O1)N1CCNCC1]

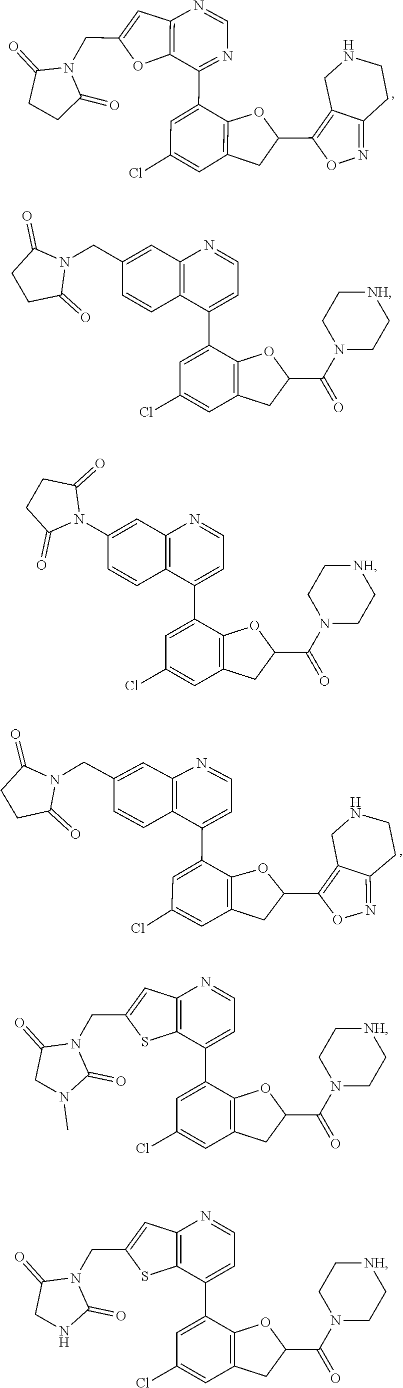 [SMILES: O=C1CCC(=O)N1Cc1cc2ncnc(-c3cc(Cl)cc4c3OC(c3onc5c3CNCC5)C4)c2o1 ; O=C(C1Cc2cc(Cl)cc(-c3ccnc4cc(CN5C(=O)CCC5=O)ccc34)c2O1)N1CCNCC1 ; O=C(C1Cc2cc(Cl)cc(-c3ccnc4cc(N5C(=O)CCC5=O)ccc34)c2O1)N1CCNCC1 ; O=C1CCC(=O)N1Cc1ccc2c(-c3cc(Cl)cc4c3OC(c3onc5c3CNCC5)C4)ccnc2c1 ; CN1CC(=O)N(Cc2cc3nccc(-c4cc(Cl)cc5c4OC(C(=O)N4CCNCC4)C5)c3s2)C1=O ; O=C(C1Cc2cc(Cl)cc(-c3ccnc4cc(CN5C(=O)CNC5=O)sc34)c2O1)N1CCNCC1]

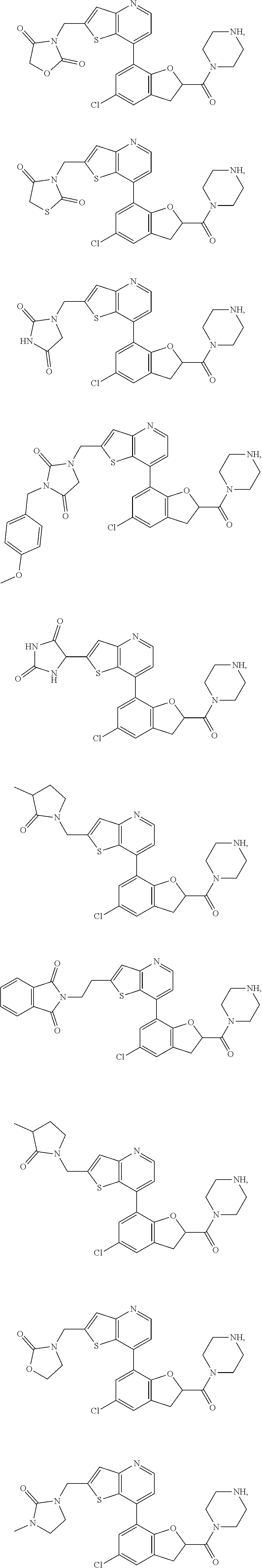

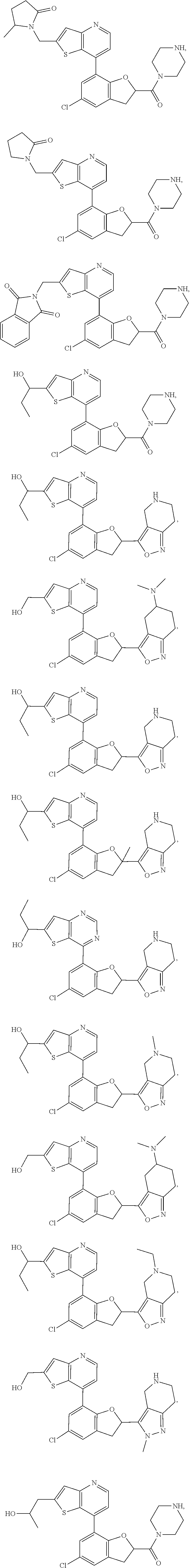

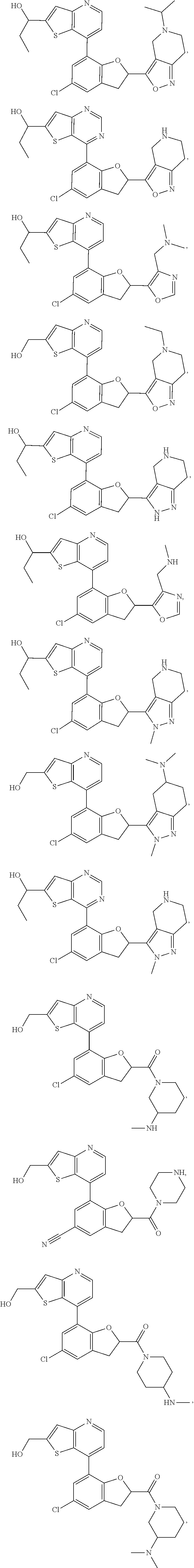

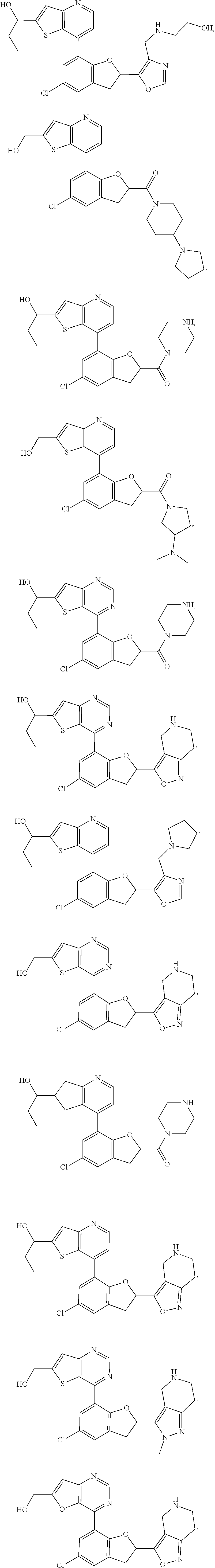

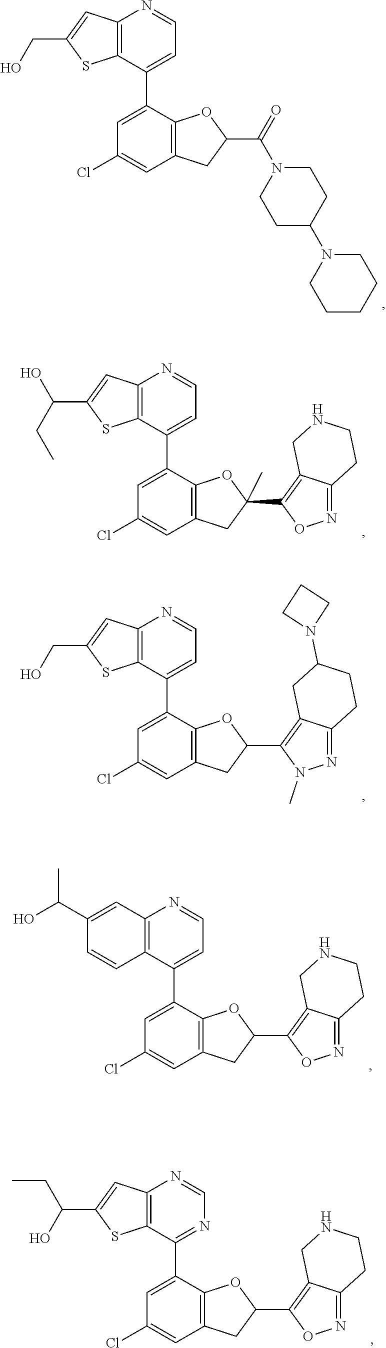 [SMILES: O=C(C1Cc2cc(Cl)cc(-c3ccnc4cc(CO)sc34)c2O1)N1CCC(N2CCCCC2)CC1 ; CCC(O)c1cc2nccc(-c3cc(Cl)cc4c3O[C@@](C)(c3onc5c3CNCC5)C4)c2s1 ; Cn1nc2c(c1C1Cc3cc(Cl)cc(-c4ccnc5cc(CO)sc45)c3O1)CC(N1CCC1)CC2 ; CC(O)c1ccc2c(-c3cc(Cl)cc4c3OC(c3onc5c3CNCC5)C4)ccnc2c1 ; CCC(O)c1cc2ncnc(-c3cc(Cl)cc4c3OC(c3onc5c3CNCC5)C4)c2s1]

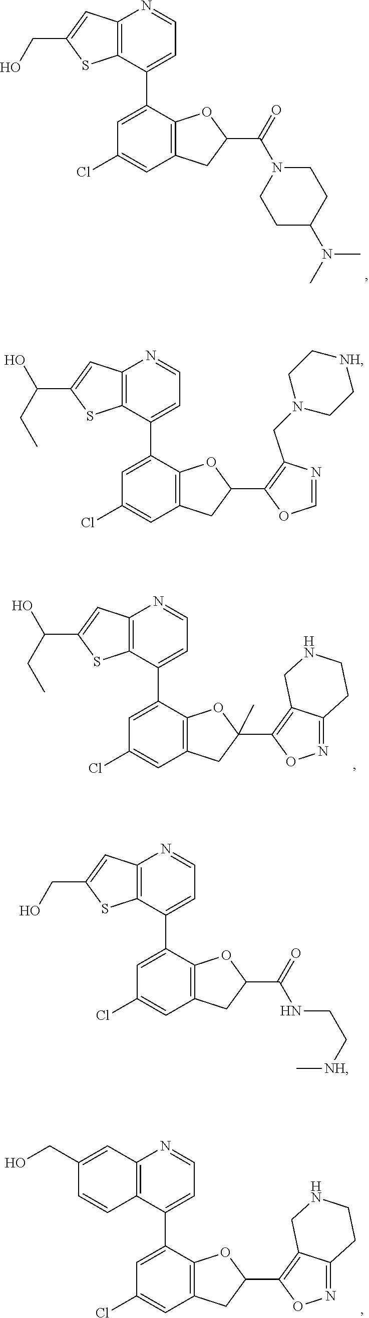 [SMILES: CN(C)C1CCN(C(=O)C2Cc3cc(Cl)cc(-c4ccnc5cc(CO)sc45)c3O2)CC1 ; CCC(O)c1cc2nccc(-c3cc(Cl)cc4c3OC(c3ocnc3CN3CCNCC3)C4)c2s1 ; CCC(O)c1cc2nccc(-c3cc(Cl)cc4c3OC(C)(c3onc5c3CNCC5)C4)c2s1 ; CNCCNC(=O)C1Cc2cc(Cl)cc(-c3ccnc4cc(CO)sc34)c2O1 ; OCc1ccc2c(-c3cc(Cl)cc4c3OC(c3onc5c3CNCC5)C4)ccnc2c1]

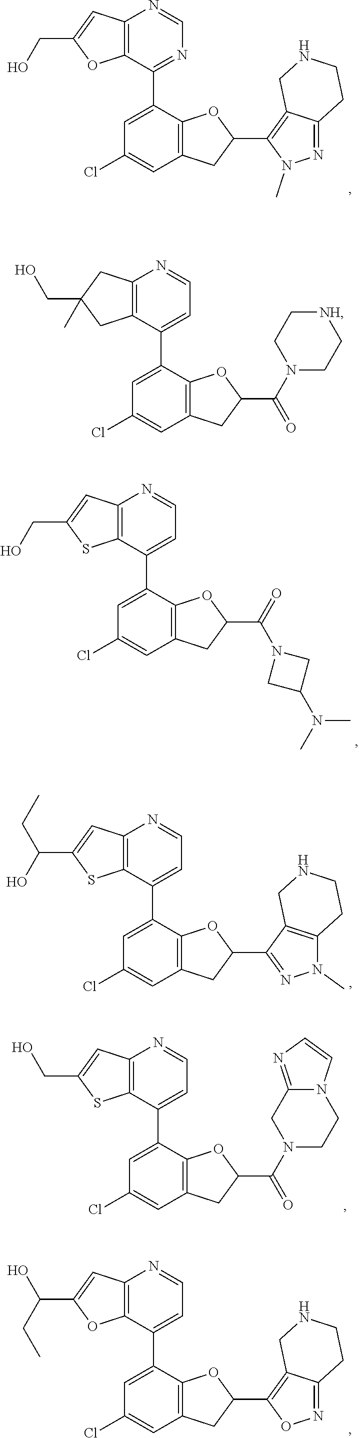 [SMILES: Cn1nc2c(c1C1Cc3cc(Cl)cc(-c4ncnc5cc(CO)oc45)c3O1)CNCC2 ; CC1(CO)Cc2nccc(-c3cc(Cl)cc4c3OC(C(=O)N3CCNCC3)C4)c2C1 ; CN(C)C1CN(C(=O)C2Cc3cc(Cl)cc(-c4ccnc5cc(CO)sc45)c3O2)C1 ; CCC(O)c1cc2nccc(-c3cc(Cl)cc4c3OC(c3nn(C)c5c3CNCC5)C4)c2s1 ; O=C(C1Cc2cc(Cl)cc(-c3ccnc4cc(CO)sc34)c2O1)N1CCn2ccnc2C1 ; CCC(O)c1cc2nccc(-c3cc(Cl)cc4c3OC(c3onc5c3CNCC5)C4)c2o1]

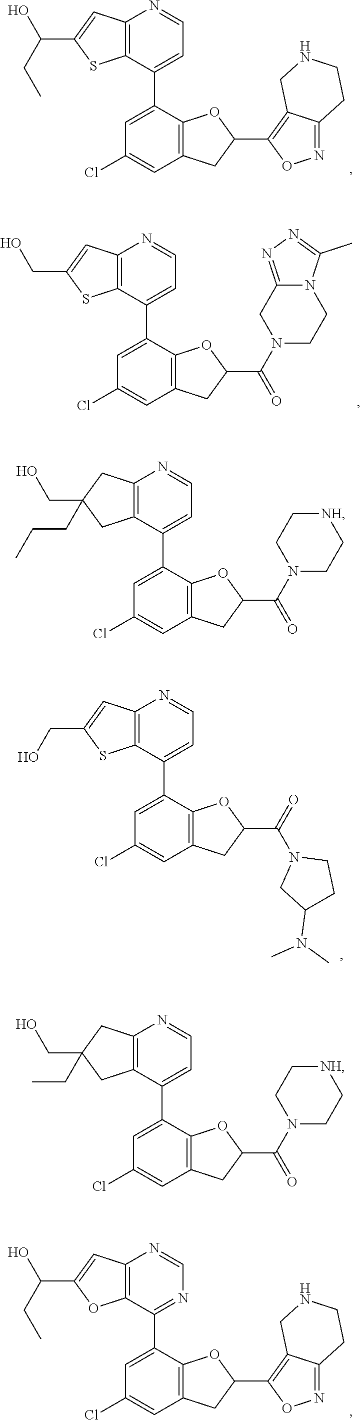

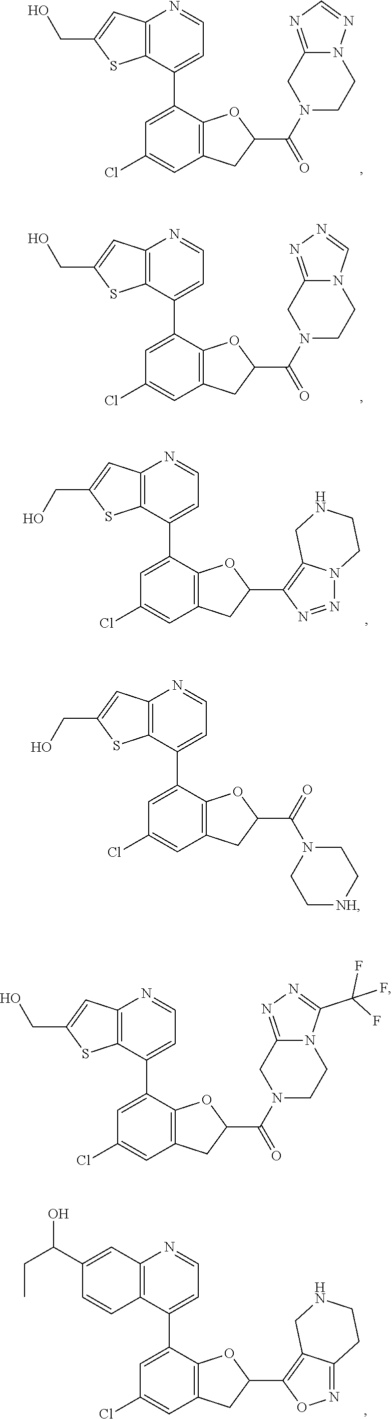 [SMILES: O=C(C1Cc2cc(Cl)cc(-c3ccnc4cc(CO)sc34)c2O1)N1CCn2ncnc2C1 ; O=C(C1Cc2cc(Cl)cc(-c3ccnc4cc(CO)sc34)c2O1)N1CCn2cnnc2C1 ; OCc1cc2nccc(-c3cc(Cl)cc4c3OC(c3nnn5c3CNCC5)C4)c2s1 ; O=C(C1Cc2cc(Cl)cc(-c3ccnc4cc(CO)sc34)c2O1)N1CCNCC1 ; O=C(C1Cc2cc(Cl)cc(-c3ccnc4cc(CO)sc34)c2O1)N1CCn2c(nnc2C(F)(F)F)C1 ; CCC(O)c1ccc2c(-c3cc(Cl)cc4c3OC(c3onc5c3CNCC5)C4)ccnc2c1]

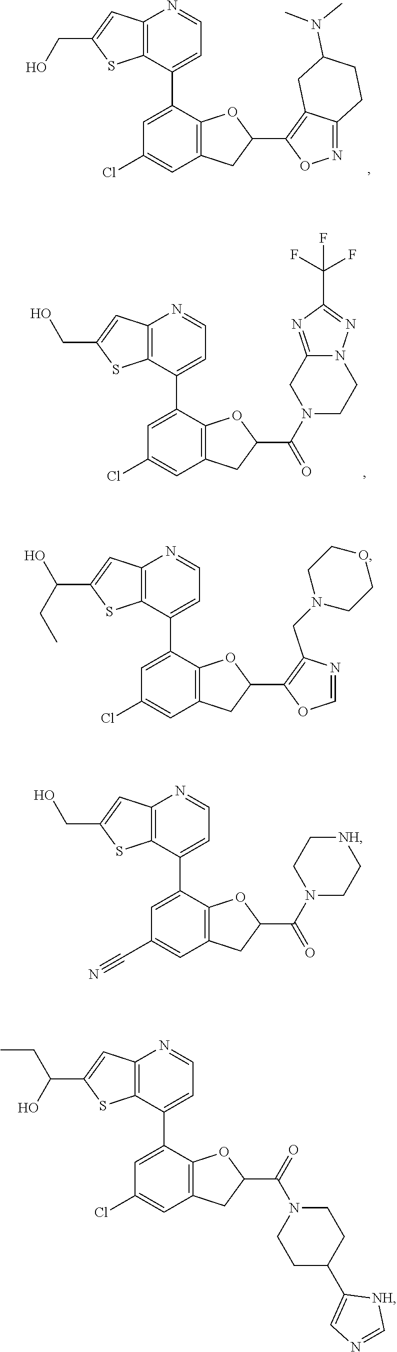 [SMILES: CN(C)C1CCc2noc(C3Cc4cc(Cl)cc(-c5ccnc6cc(CO)sc56)c4O3)c2C1 ; O=C(C1Cc2cc(Cl)cc(-c3ccnc4cc(CO)sc34)c2O1)N1CCn2nc(C(F)(F)F)nc2C1 ; CCC(O)c1cc2nccc(-c3cc(Cl)cc4c3OC(c3ocnc3CN3CCOCC3)C4)c2s1 ; N#Cc1cc2c(c(-c3ccnc4cc(CO)sc34)c1)OC(C(=O)N1CCNCC1)C2 ; CCC(O)c1cc2nccc(-c3cc(Cl)cc4c3OC(C(=O)N3CCC(c5cnc[nH]5)CC3)C4)c2s1]

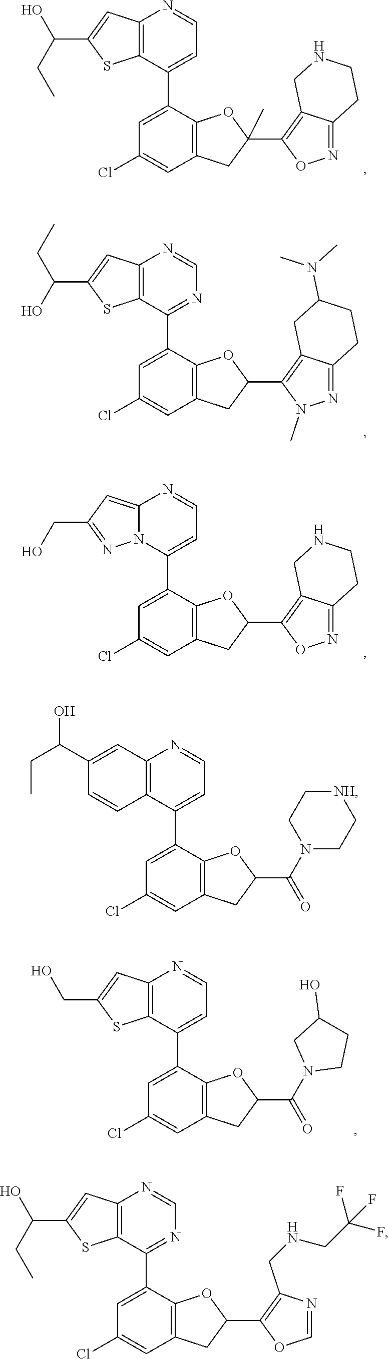 [SMILES: CCC(O)c1cc2nccc(-c3cc(Cl)cc4c3OC(C)(c3onc5c3CNCC5)C4)c2s1 ; CCC(O)c1cc2ncnc(-c3cc(Cl)cc4c3OC(c3c5c(nn3C)CCC(N(C)C)C5)C4)c2s1 ; OCc1cc2nccc(-c3cc(Cl)cc4c3OC(c3onc5c3CNCC5)C4)n2n1 ; CCC(O)c1ccc2c(-c3cc(Cl)cc4c3OC(C(=O)N3CCNCC3)C4)ccnc2c1 ; O=C(C1Cc2cc(Cl)cc(-c3ccnc4cc(CO)sc34)c2O1)N1CCC(O)C1 ; CCC(O)c1cc2ncnc(-c3cc(Cl)cc4c3OC(c3ocnc3CNCC(F)(F)F)C4)c2s1]

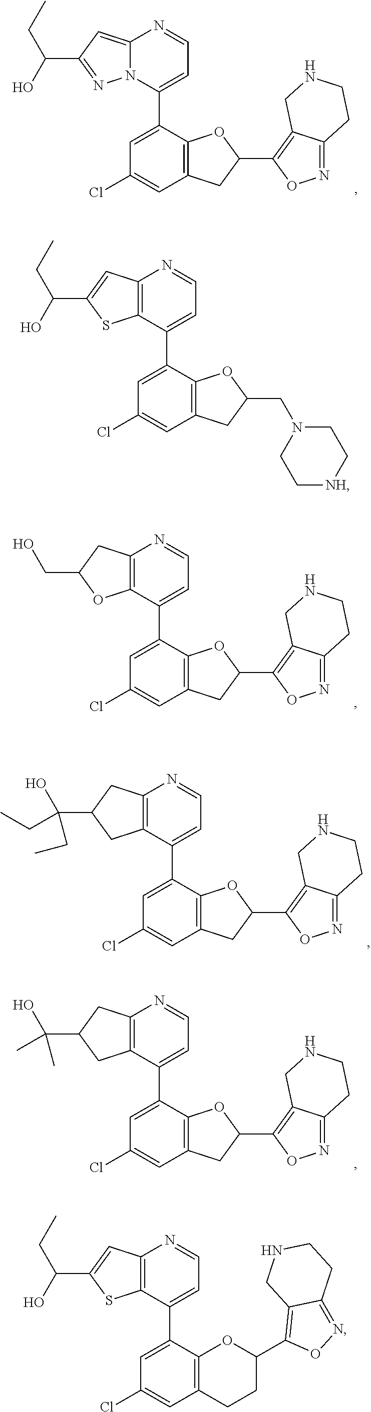 [SMILES: CCC(O)c1cc2nccc(-c3cc(Cl)cc4c3OC(c3onc5c3CNCC5)C4)n2n1 ; CCC(O)c1cc2nccc(-c3cc(Cl)cc4c3OC(CN3CCNCC3)C4)c2s1 ; OCC1Cc2nccc(-c3cc(Cl)cc4c3OC(c3onc5c3CNCC5)C4)c2O1 ; CCC(O)(CC)C1Cc2nccc(-c3cc(Cl)cc4c3OC(c3onc5c3CNCC5)C4)c2C1 ; CC(C)(O)C1Cc2nccc(-c3cc(Cl)cc4c3OC(c3onc5c3CNCC5)C4)c2C1 ; CCC(O)c1cc2nccc(-c3cc(Cl)cc4c3OC(c3onc5c3CNCC5)CC4)c2s1]

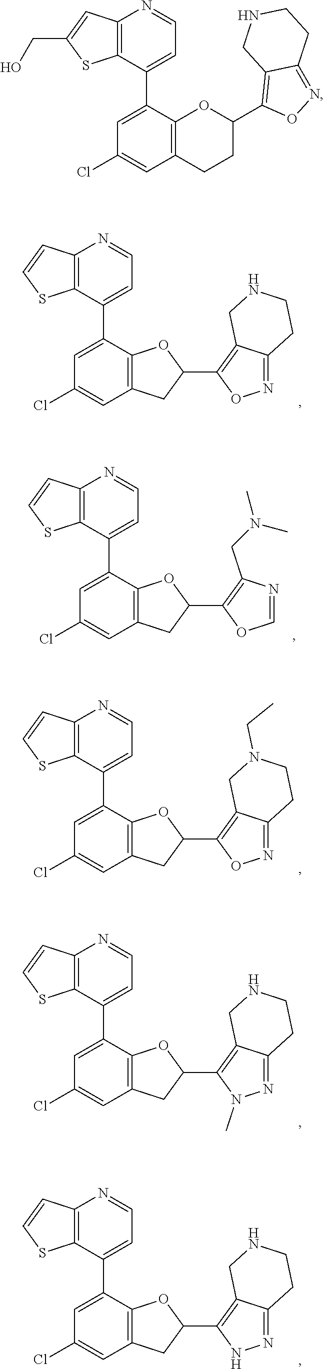

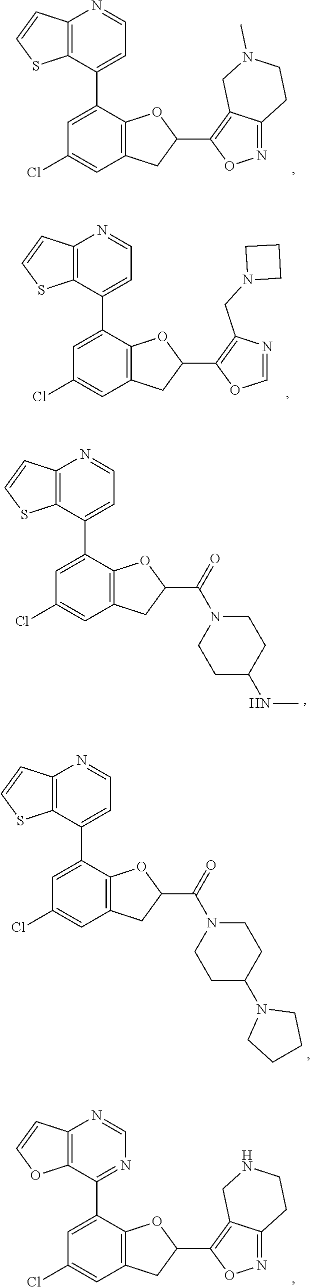 [SMILES: CN1CCc2noc(C3Cc4cc(Cl)cc(-c5ccnc6ccsc56)c4O3)c2C1 ; Clc1cc2c(c(-c3ccnc4ccsc34)c1)OC(c1ocnc1CN1CCC1)C2 ; CNC1CCN(C(=O)C2Cc3cc(Cl)cc(-c4ccnc5ccsc45)c3O2)CC1 ; O=C(C1Cc2cc(Cl)cc(-c3ccnc4ccsc34)c2O1)N1CCC(N2CCCC2)CC1 ; Clc1cc2c(c(-c3ncnc4ccoc34)c1)OC(c1onc3c1CNCC3)C2]

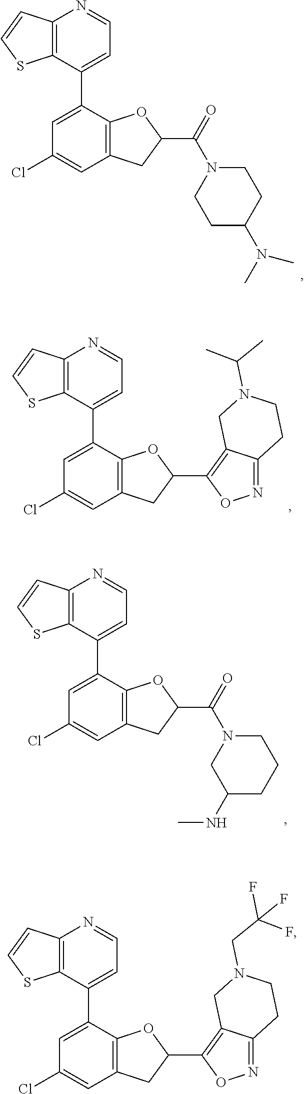 [SMILES: CN(C)C1CCN(C(=O)C2Cc3cc(Cl)cc(-c4ccnc5ccsc45)c3O2)CC1 ; CC(C)N1CCc2noc(C3Cc4cc(Cl)cc(-c5ccnc6ccsc56)c4O3)c2C1 ; CNC1CCCN(C(=O)C2Cc3cc(Cl)cc(-c4ccnc5ccsc45)c3O2)C1 ; FC(F)(F)CN1CCc2noc(C3Cc4cc(Cl)cc(-c5ccnc6ccsc56)c4O3)c2C1]

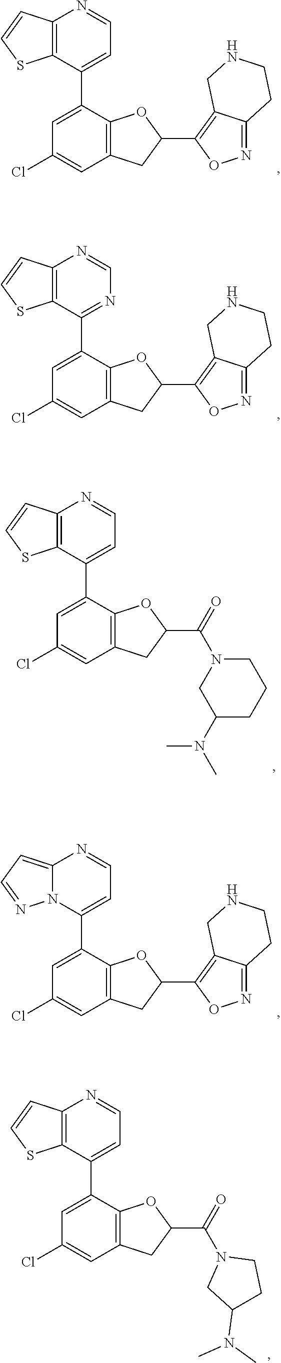 [SMILES: Clc1cc2c(c(-c3ccnc4ccsc34)c1)OC(c1onc3c1CNCC3)C2 ; Clc1cc2c(c(-c3ncnc4ccsc34)c1)OC(c1onc3c1CNCC3)C2 ; CN(C)C1CCCN(C(=O)C2Cc3cc(Cl)cc(-c4ccnc5ccsc45)c3O2)C1 ; Clc1cc2c(c(-c3ccnc4ccnn34)c1)OC(c1onc3c1CNCC3)C2 ; CN(C)C1CCN(C(=O)C2Cc3cc(Cl)cc(-c4ccnc5ccsc45)c3O2)C1]

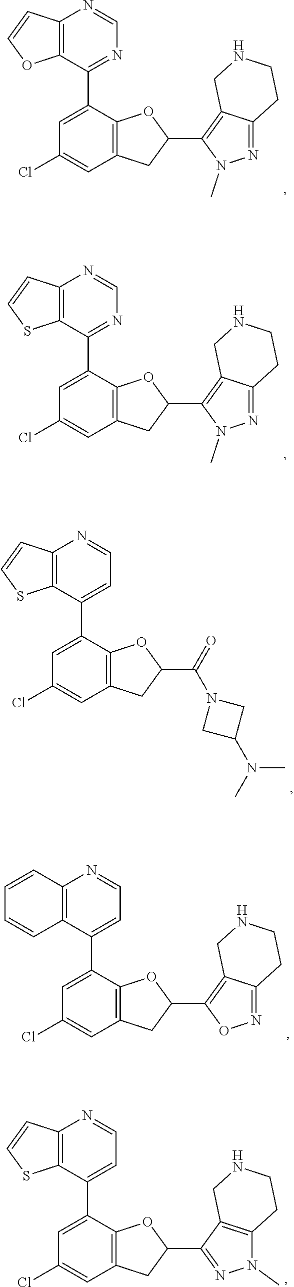 [SMILES: Cn1nc2c(c1C1Cc3cc(Cl)cc(-c4ncnc5ccoc45)c3O1)CNCC2 ; Cn1nc2c(c1C1Cc3cc(Cl)cc(-c4ncnc5ccsc45)c3O1)CNCC2 ; CN(C)C1CN(C(=O)C2Cc3cc(Cl)cc(-c4ccnc5ccsc45)c3O2)C1 ; Clc1cc2c(c(-c3ccnc4ccccc34)c1)OC(c1onc3c1CNCC3)C2 ; Cn1nc(C2Cc3cc(Cl)cc(-c4ccnc5ccsc45)c3O2)c2c1CCNC2]

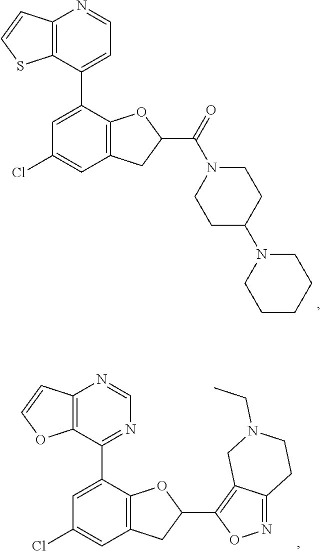 [SMILES: O=C(C1Cc2cc(Cl)cc(-c3ccnc4ccsc34)c2O1)N1CCC(N2CCCCC2)CC1 ; CCN1CCc2noc(C3Cc4cc(Cl)cc(-c5ncnc6ccoc56)c4O3)c2C1]

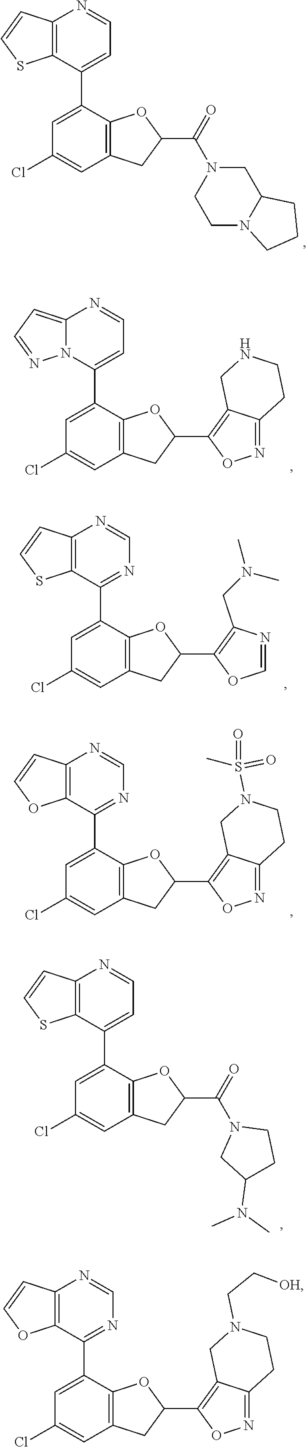

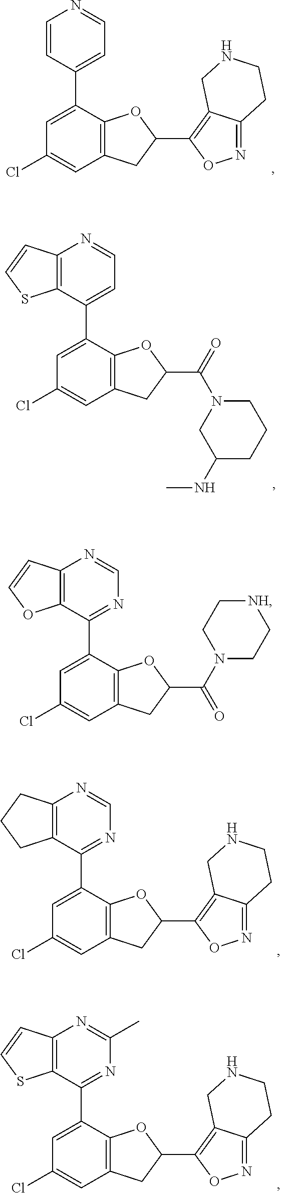 [SMILES: Clc1cc2c(c(-c3ccncc3)c1)OC(c1onc3c1CNCC3)C2 ; CNC1CCCN(C(=O)C2Cc3cc(Cl)cc(-c4ccnc5ccsc45)c3O2)C1 ; O=C(C1Cc2cc(Cl)cc(-c3ncnc4ccoc34)c2O1)N1CCNCC1 ; Clc1cc2c(c(-c3ncnc4c3CCC4)c1)OC(c1onc3c1CNCC3)C2 ; Cc1nc(-c2cc(Cl)cc3c2OC(c2onc4c2CNCC4)C3)c2sccc2n1]

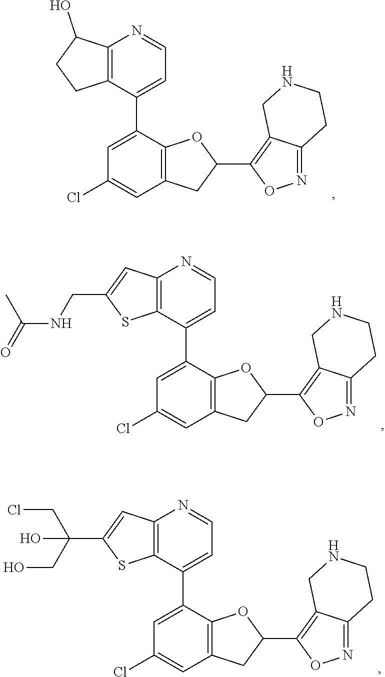 [SMILES: OC1CCc2c(-c3cc(Cl)cc4c3OC(c3onc5c3CNCC5)C4)ccnc21 ; CC(=O)NCc1cc2nccc(-c3cc(Cl)cc4c3OC(c3onc5c3CNCC5)C4)c2s1 ; OCC(O)(CCl)c1cc2nccc(-c3cc(Cl)cc4c3OC(c3onc5c3CNCC5)C4)c2s1]

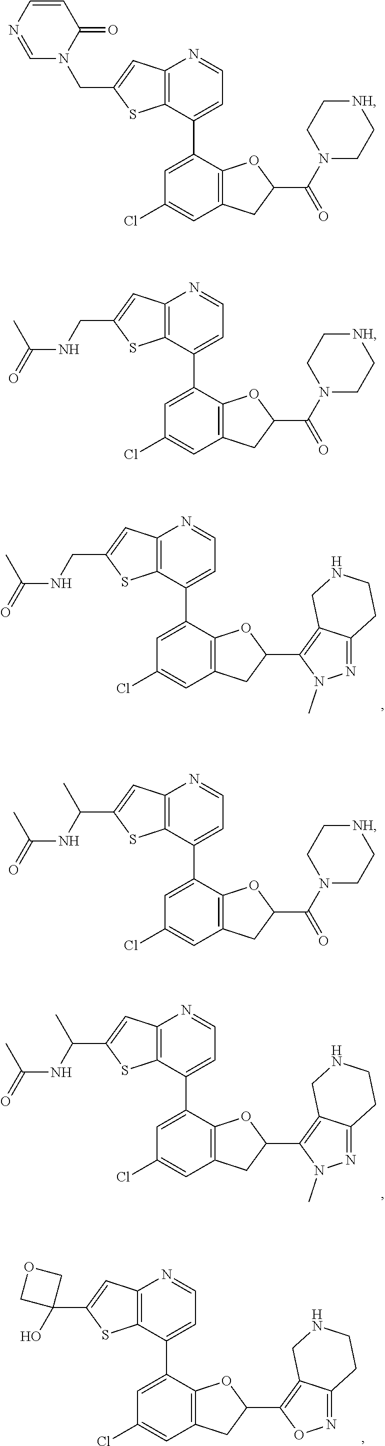 [SMILES: O=C(C1Cc2cc(Cl)cc(-c3ccnc4cc(Cn5cnccc5=O)sc34)c2O1)N1CCNCC1 ; CC(=O)NCc1cc2nccc(-c3cc(Cl)cc4c3OC(C(=O)N3CCNCC3)C4)c2s1 ; CC(=O)NCc1cc2nccc(-c3cc(Cl)cc4c3OC(c3c5c(nn3C)CCNC5)C4)c2s1 ; CC(=O)NC(C)c1cc2nccc(-c3cc(Cl)cc4c3OC(C(=O)N3CCNCC3)C4)c2s1 ; CC(=O)NC(C)c1cc2nccc(-c3cc(Cl)cc4c3OC(c3c5c(nn3C)CCNC5)C4)c2s1 ; OC1(c2cc3nccc(-c4cc(Cl)cc5c4OC(c4onc6c4CNCC6)C5)c3s2)COC1]

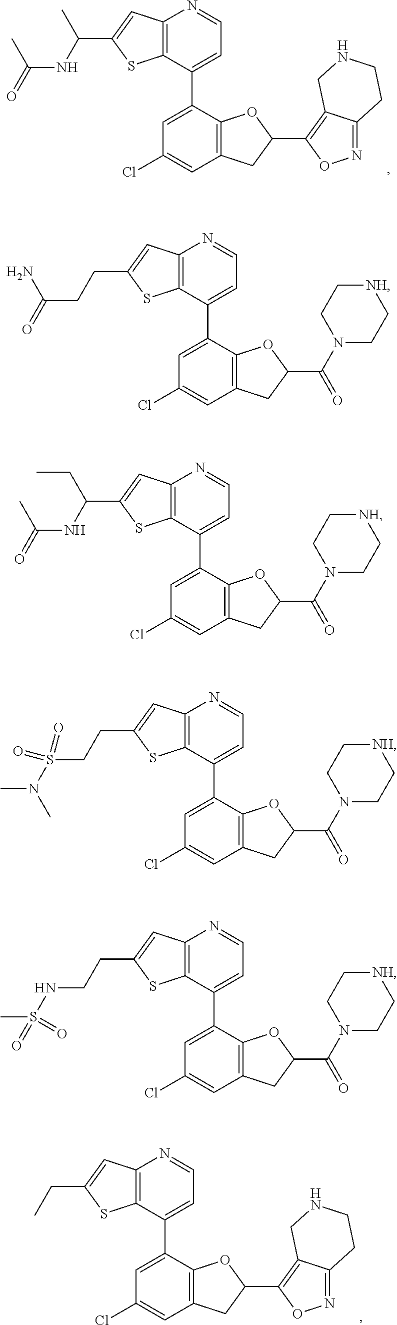 [SMILES: CC(=O)NC(C)c1cc2nccc(-c3cc(Cl)cc4c3OC(c3onc5c3CNCC5)C4)c2s1 ; NC(=O)CCc1cc2nccc(-c3cc(Cl)cc4c3OC(C(=O)N3CCNCC3)C4)c2s1 ; CCC(NC(C)=O)c1cc2nccc(-c3cc(Cl)cc4c3OC(C(=O)N3CCNCC3)C4)c2s1 ; CN(C)S(=O)(=O)CCc1cc2nccc(-c3cc(Cl)cc4c3OC(C(=O)N3CCNCC3)C4)c2s1 ; CS(=O)(=O)NCCc1cc2nccc(-c3cc(Cl)cc4c3OC(C(=O)N3CCNCC3)C4)c2s1 ; CCc1cc2nccc(-c3cc(Cl)cc4c3OC(c3onc5c3CNCC5)C4)c2s1]

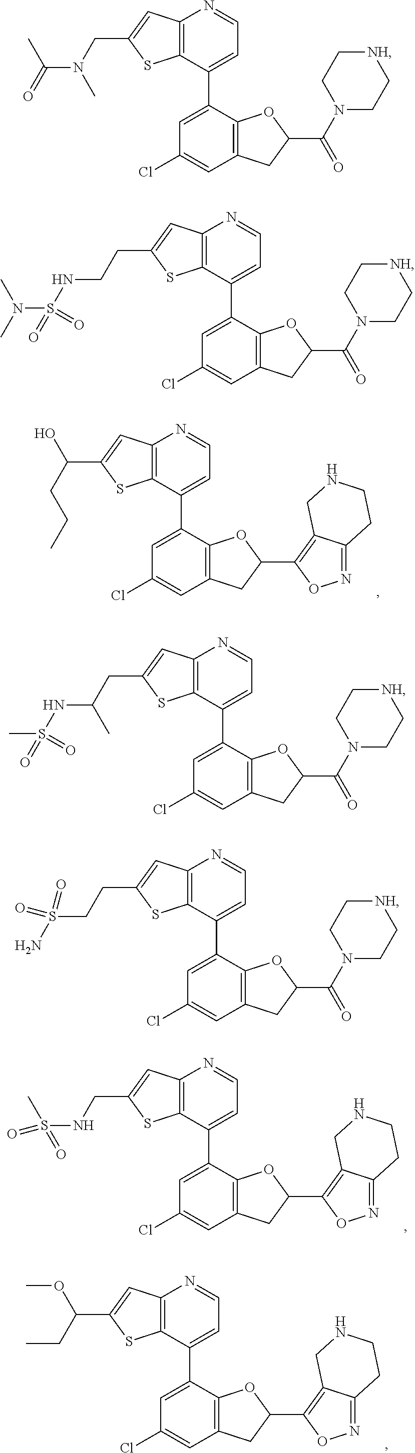

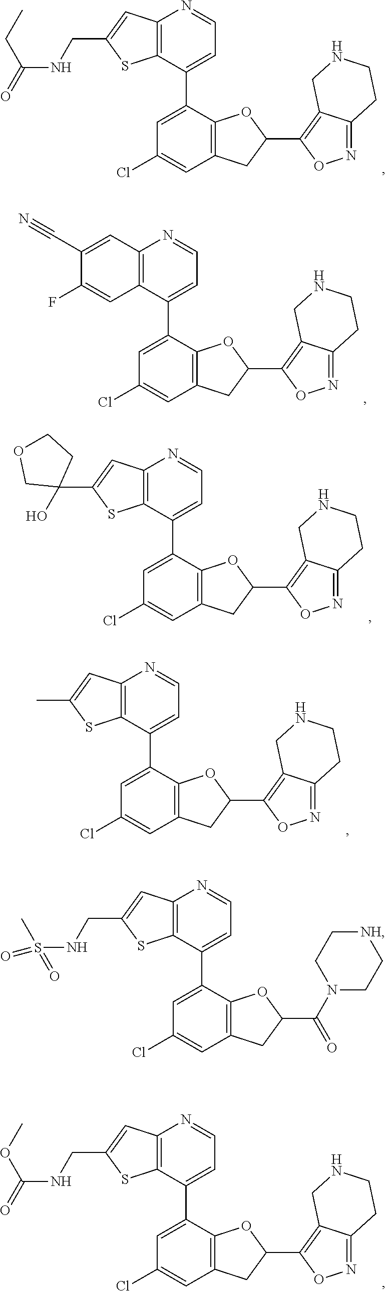 [SMILES: CCC(=O)NCc1cc2nccc(-c3cc(Cl)cc4c3OC(c3onc5c3CNCC5)C4)c2s1 ; N#Cc1cc2nccc(-c3cc(Cl)cc4c3OC(c3onc5c3CNCC5)C4)c2cc1F ; OC1(c2cc3nccc(-c4cc(Cl)cc5c4OC(c4onc6c4CNCC6)C5)c3s2)CCOC1 ; Cc1cc2nccc(-c3cc(Cl)cc4c3OC(c3onc5c3CNCC5)C4)c2s1 ; CS(=O)(=O)NCc1cc2nccc(-c3cc(Cl)cc4c3OC(C(=O)N3CCNCC3)C4)c2s1 ; COC(=O)NCc1cc2nccc(-c3cc(Cl)cc4c3OC(c3onc5c3CNCC5)C4)c2s1]

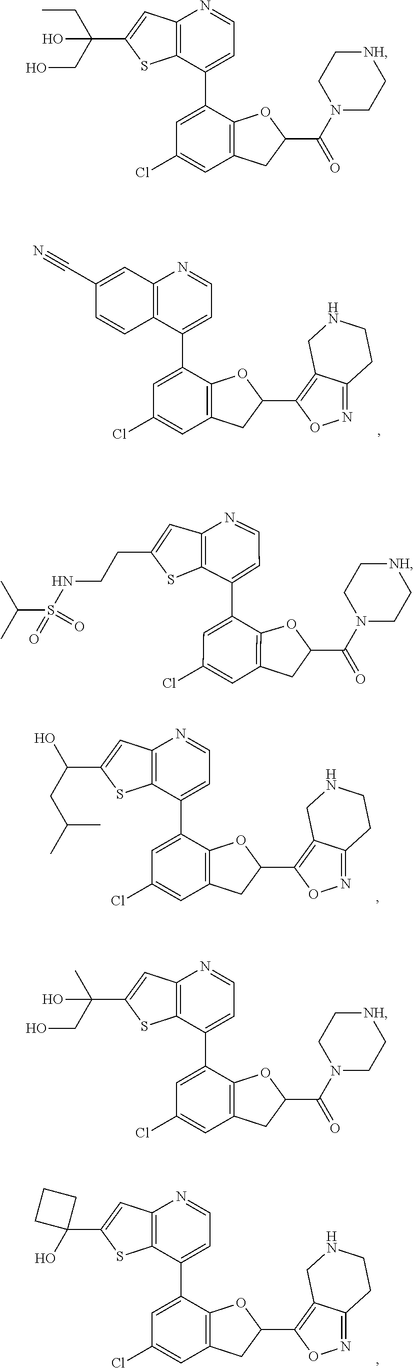 [SMILES: CCC(O)(CO)c1cc2nccc(-c3cc(Cl)cc4c3OC(C(=O)N3CCNCC3)C4)c2s1 ; N#Cc1ccc2c(-c3cc(Cl)cc4c3OC(c3onc5c3CNCC5)C4)ccnc2c1 ; CC(C)S(=O)(=O)NCCc1cc2nccc(-c3cc(Cl)cc4c3OC(C(=O)N3CCNCC3)C4)c2s1 ; CC(C)CC(O)c1cc2nccc(-c3cc(Cl)cc4c3OC(c3onc5c3CNCC5)C4)c2s1 ; CC(O)(CO)c1cc2nccc(-c3cc(Cl)cc4c3OC(C(=O)N3CCNCC3)C4)c2s1 ; OC1(c2cc3nccc(-c4cc(Cl)cc5c4OC(c4onc6c4CNCC6)C5)c3s2)CCC1]

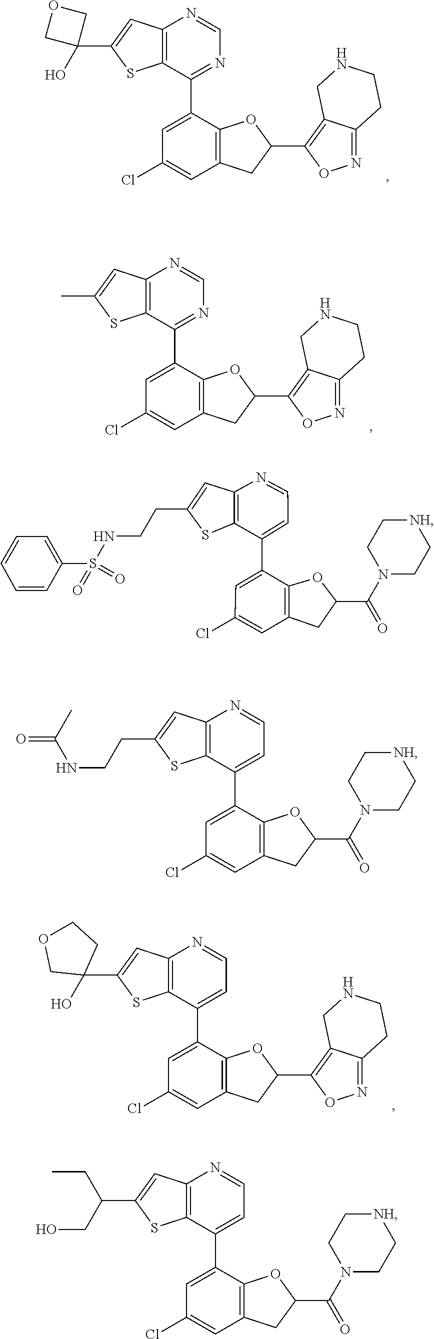 [SMILES: OC1(c2cc3ncnc(-c4cc(Cl)cc5c4OC(c4onc6c4CNCC6)C5)c3s2)COC1 ; Cc1cc2ncnc(-c3cc(Cl)cc4c3OC(c3onc5c3CNCC5)C4)c2s1 ; O=C(C1Cc2cc(Cl)cc(-c3ccnc4cc(CCNS(=O)(=O)c5ccccc5)sc34)c2O1)N1CCNCC1 ; CC(=O)NCCc1cc2nccc(-c3cc(Cl)cc4c3OC(C(=O)N3CCNCC3)C4)c2s1 ; OC1(c2cc3nccc(-c4cc(Cl)cc5c4OC(c4onc6c4CNCC6)C5)c3s2)CCOC1 ; CCC(CO)c1cc2nccc(-c3cc(Cl)cc4c3OC(C(=O)N3CCNCC3)C4)c2s1]

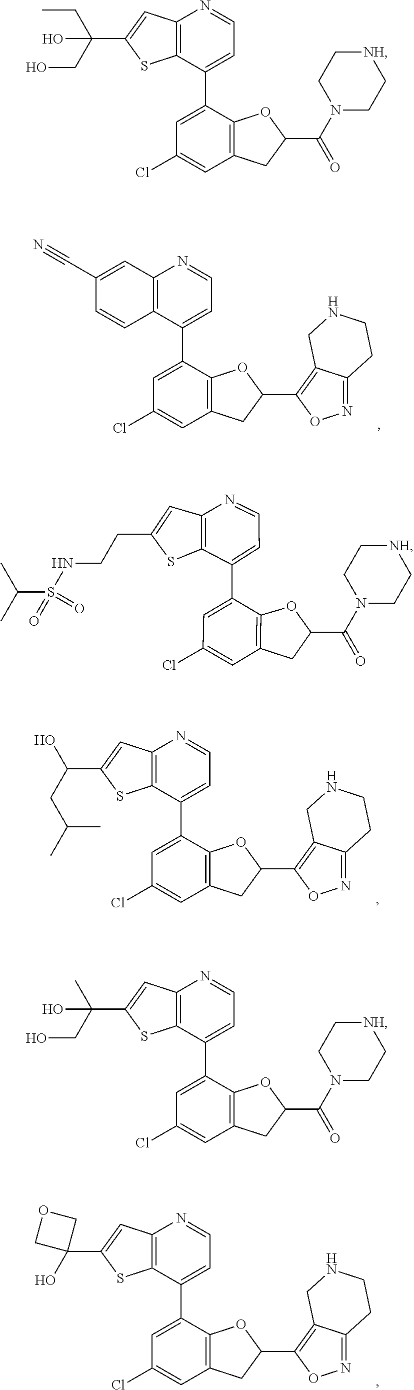 [SMILES: CCC(O)(CO)c1cc2nccc(-c3cc(Cl)cc4c3OC(C(=O)N3CCNCC3)C4)c2s1 ; N#Cc1ccc2c(-c3cc(Cl)cc4c3OC(c3onc5c3CNCC5)C4)ccnc2c1 ; CC(C)S(=O)(=O)NCCc1cc2nccc(-c3cc(Cl)cc4c3OC(C(=O)N3CCNCC3)C4)c2s1 ; CC(C)CC(O)c1cc2nccc(-c3cc(Cl)cc4c3OC(c3onc5c3CNCC5)C4)c2s1 ; CC(O)(CO)c1cc2nccc(-c3cc(Cl)cc4c3OC(C(=O)N3CCNCC3)C4)c2s1 ; OC1(c2cc3nccc(-c4cc(Cl)cc5c4OC(c4onc6c4CNCC6)C5)c3s2)COC1]

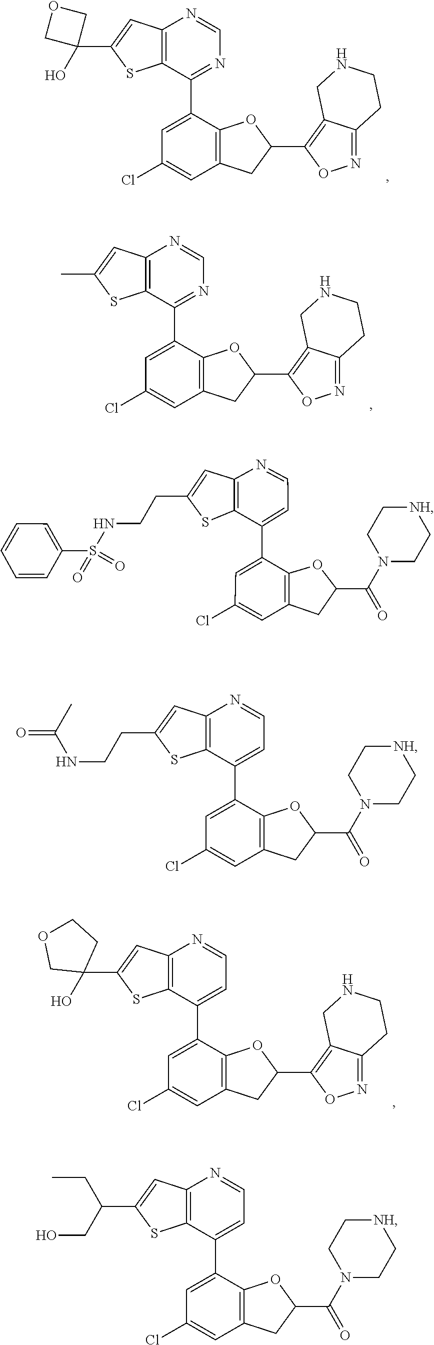 [SMILES: OC1(c2cc3ncnc(-c4cc(Cl)cc5c4OC(c4onc6c4CNCC6)C5)c3s2)COC1 ; Cc1cc2ncnc(-c3cc(Cl)cc4c3OC(c3onc5c3CNCC5)C4)c2s1 ; O=C(C1Cc2cc(Cl)cc(-c3ccnc4cc(CCNS(=O)(=O)c5ccccc5)sc34)c2O1)N1CCNCC1 ; CC(=O)NCCc1cc2nccc(-c3cc(Cl)cc4c3OC(C(=O)N3CCNCC3)C4)c2s1 ; OC1(c2cc3nccc(-c4cc(Cl)cc5c4OC(c4onc6c4CNCC6)C5)c3s2)CCOC1 ; CCC(CO)c1cc2nccc(-c3cc(Cl)cc4c3OC(C(=O)N3CCNCC3)C4)c2s1]

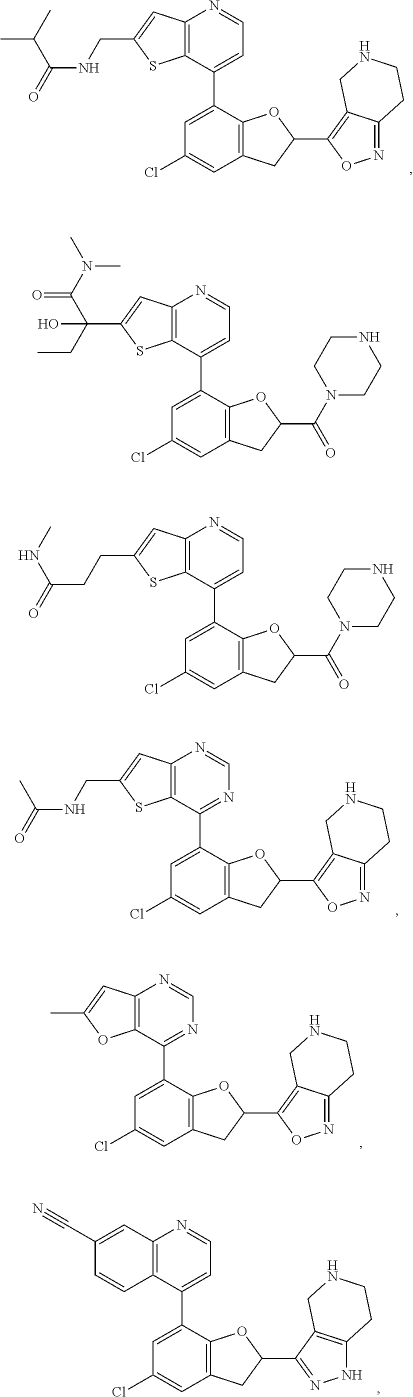 [SMILES: CC(C)C(=O)NCc1cc2nccc(-c3cc(Cl)cc4c3OC(c3onc5c3CNCC5)C4)c2s1 ; CCC(O)(C(=O)N(C)C)c1cc2nccc(-c3cc(Cl)cc4c3OC(C(=O)N3CCNCC3)C4)c2s1 ; CNC(=O)CCc1cc2nccc(-c3cc(Cl)cc4c3OC(C(=O)N3CCNCC3)C4)c2s1 ; CC(=O)NCc1cc2ncnc(-c3cc(Cl)cc4c3OC(c3onc5c3CNCC5)C4)c2s1 ; Cc1cc2ncnc(-c3cc(Cl)cc4c3OC(c3onc5c3CNCC5)C4)c2o1 ; N#Cc1ccc2c(-c3cc(Cl)cc4c3OC(c3n[nH]c5c3CNCC5)C4)ccnc2c1]

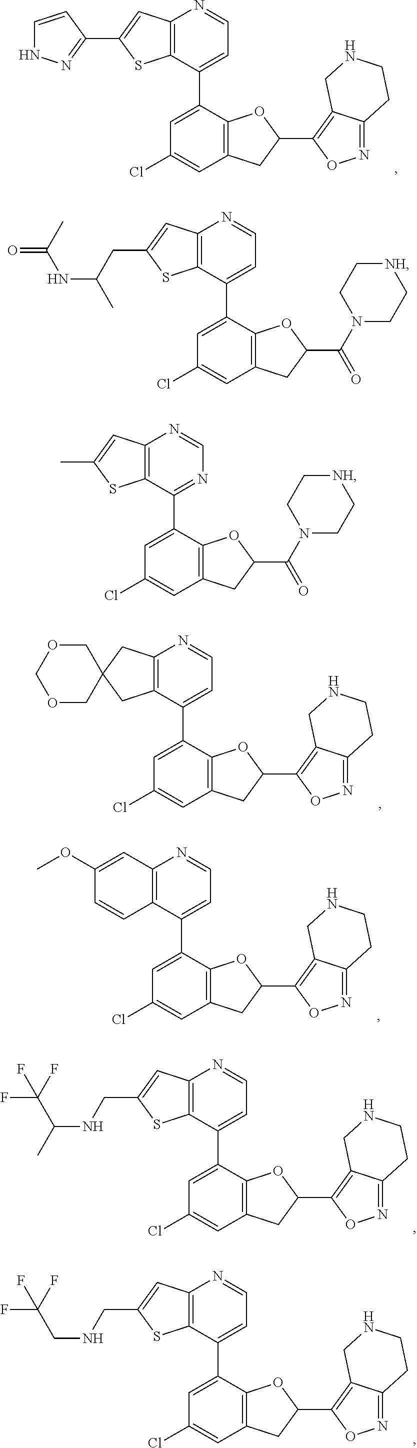

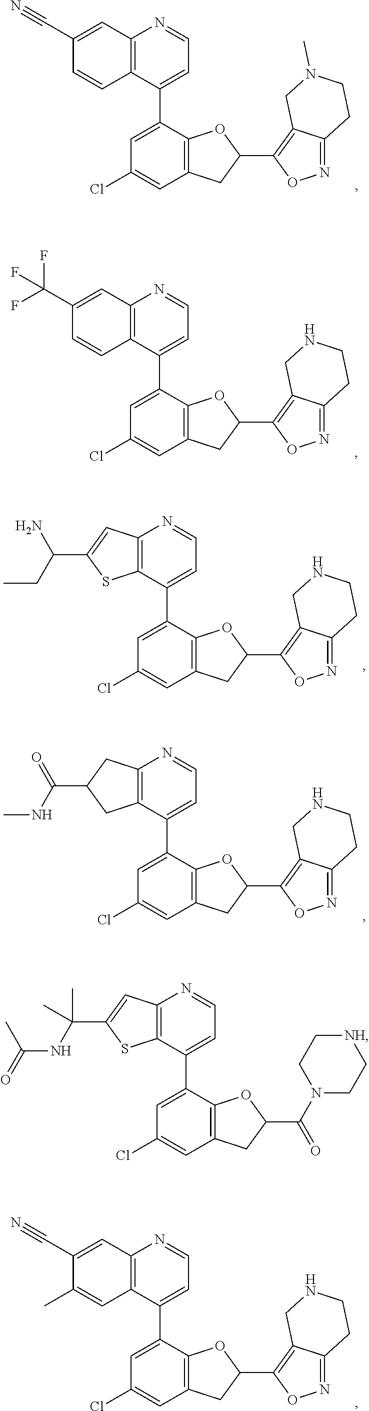 [SMILES: CN1CCc2noc(C3Cc4cc(Cl)cc(-c5ccnc6cc(C#N)ccc56)c4O3)c2C1 ; FC(F)(F)c1ccc2c(-c3cc(Cl)cc4c3OC(c3onc5c3CNCC5)C4)ccnc2c1 ; CCC(N)c1cc2nccc(-c3cc(Cl)cc4c3OC(c3onc5c3CNCC5)C4)c2s1 ; CNC(=O)C1Cc2nccc(-c3cc(Cl)cc4c3OC(c3onc5c3CNCC5)C4)c2C1 ; CC(=O)NC(C)(C)c1cc2nccc(-c3cc(Cl)cc4c3OC(C(=O)N3CCNCC3)C4)c2s1 ; Cc1cc2c(-c3cc(Cl)cc4c3OC(c3onc5c3CNCC5)C4)ccnc2cc1C#N]

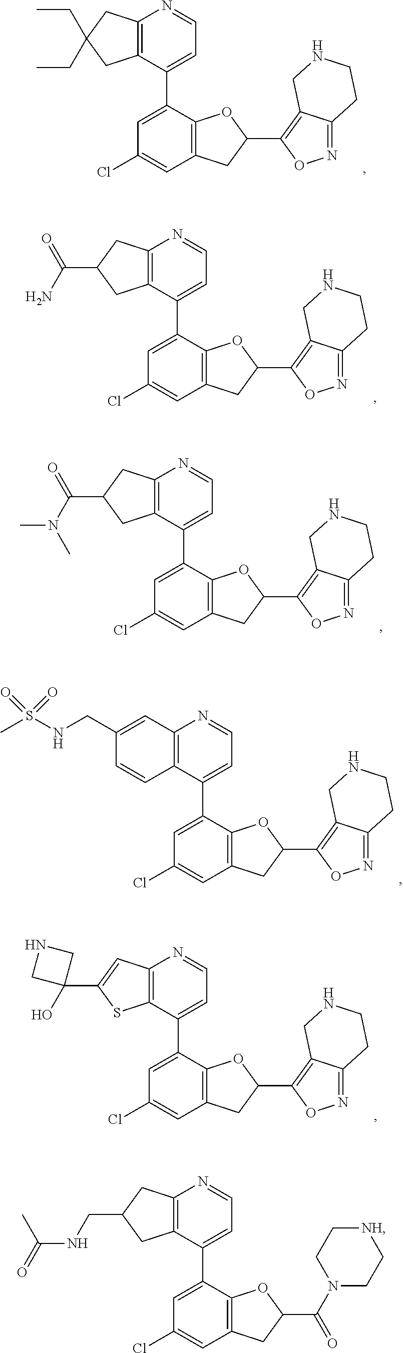 [SMILES: CCC1(CC)Cc2nccc(-c3cc(Cl)cc4c3OC(c3onc5c3CNCC5)C4)c2C1 ; NC(=O)C1Cc2nccc(-c3cc(Cl)cc4c3OC(c3onc5c3CNCC5)C4)c2C1 ; CN(C)C(=O)C1Cc2nccc(-c3cc(Cl)cc4c3OC(c3onc5c3CNCC5)C4)c2C1 ; CS(=O)(=O)NCc1ccc2c(-c3cc(Cl)cc4c3OC(c3onc5c3CNCC5)C4)ccnc2c1 ; OC1(c2cc3nccc(-c4cc(Cl)cc5c4OC(c4onc6c4CNCC6)C5)c3s2)CNC1 ; CC(=O)NCC1Cc2nccc(-c3cc(Cl)cc4c3OC(C(=O)N3CCNCC3)C4)c2C1]

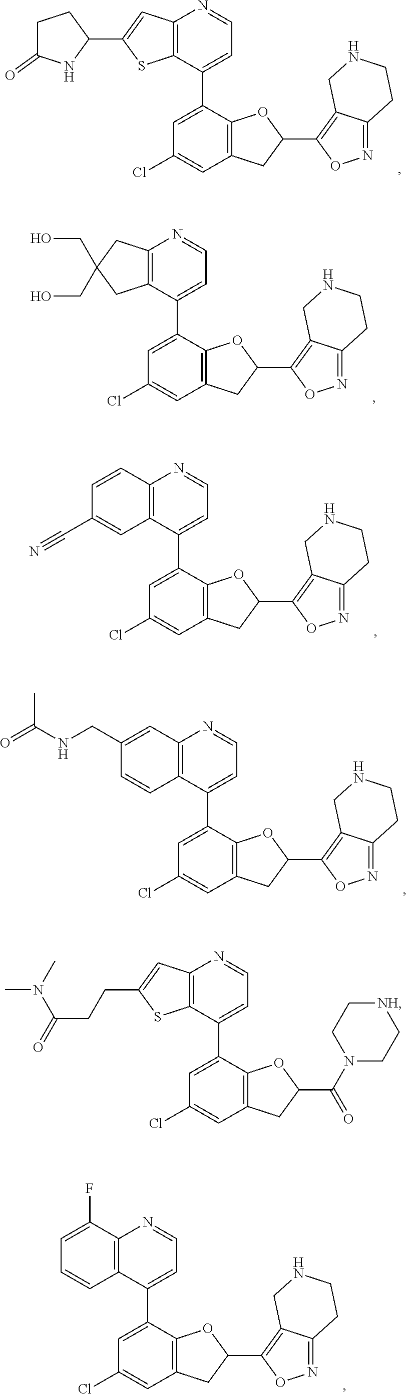 [SMILES: O=C1CCC(c2cc3nccc(-c4cc(Cl)cc5c4OC(c4onc6c4CNCC6)C5)c3s2)N1 ; OCC1(CO)Cc2nccc(-c3cc(Cl)cc4c3OC(c3onc5c3CNCC5)C4)c2C1 ; N#Cc1ccc2nccc(-c3cc(Cl)cc4c3OC(c3onc5c3CNCC5)C4)c2c1 ; CC(=O)NCc1ccc2c(-c3cc(Cl)cc4c3OC(c3onc5c3CNCC5)C4)ccnc2c1 ; CN(C)C(=O)CCc1cc2nccc(-c3cc(Cl)cc4c3OC(C(=O)N3CCNCC3)C4)c2s1 ; Fc1cccc2c(-c3cc(Cl)cc4c3OC(c3onc5c3CNCC5)C4)ccnc12]

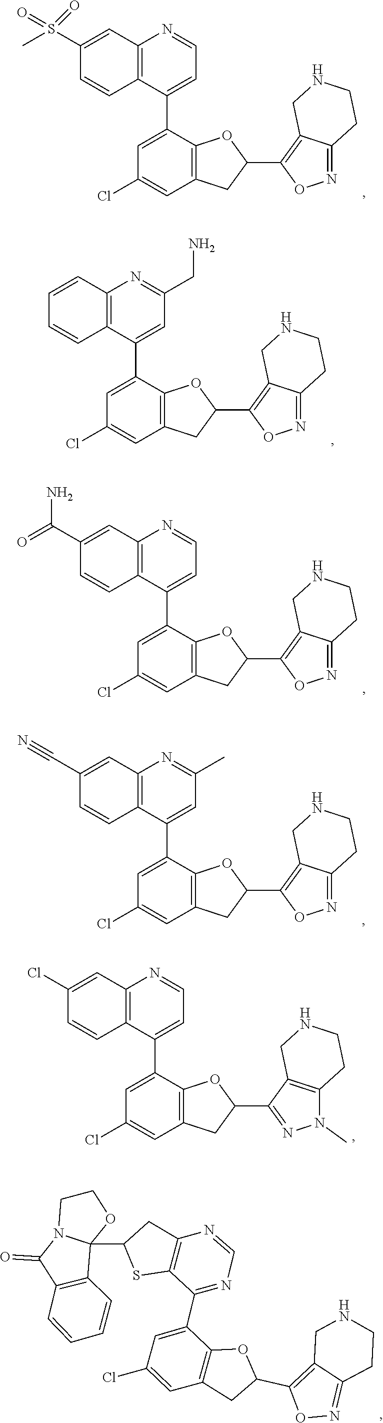 [SMILES: CS(=O)(=O)c1ccc2c(-c3cc(Cl)cc4c3OC(c3onc5c3CNCC5)C4)ccnc2c1 ; NCc1cc(-c2cc(Cl)cc3c2OC(c2onc4c2CNCC4)C3)c2ccccc2n1 ; NC(=O)c1ccc2c(-c3cc(Cl)cc4c3OC(c3onc5c3CNCC5)C4)ccnc2c1 ; Cc1cc(-c2cc(Cl)cc3c2OC(c2onc4c2CNCC4)C3)c2ccc(C#N)cc2n1 ; Cn1nc(C2Cc3cc(Cl)cc(-c4ccnc5cc(Cl)ccc45)c3O2)c2c1CCNC2 ; O=C1c2ccccc2C2(C3Cc4ncnc(-c5cc(Cl)cc6c5OC(c5onc7c5CNCC7)C6)c4S3)OCCN12]

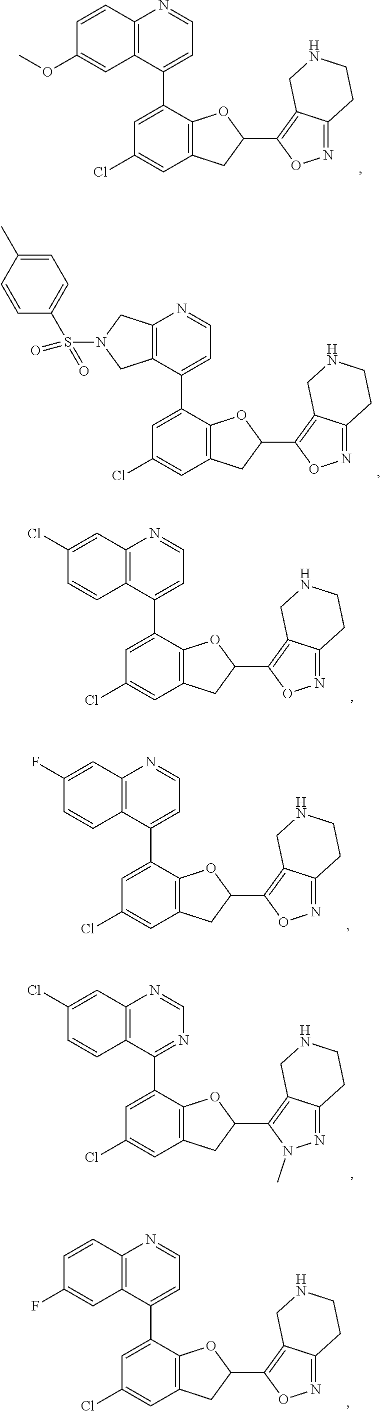 [SMILES: COc1ccc2nccc(-c3cc(Cl)cc4c3OC(c3onc5c3CNCC5)C4)c2c1 ; Cc1ccc(S(=O)(=O)N2Cc3nccc(-c4cc(Cl)cc5c4OC(c4onc6c4CNCC6)C5)c3C2)cc1 ; Clc1cc2c(c(-c3ccnc4cc(Cl)ccc34)c1)OC(c1onc3c1CNCC3)C2 ; Fc1ccc2c(-c3cc(Cl)cc4c3OC(c3onc5c3CNCC5)C4)ccnc2c1 ; Cn1nc2c(c1C1Cc3cc(Cl)cc(-c4ncnc5cc(Cl)ccc45)c3O1)CNCC2 ; Fc1ccc2nccc(-c3cc(Cl)cc4c3OC(c3onc5c3CNCC5)C4)c2c1]

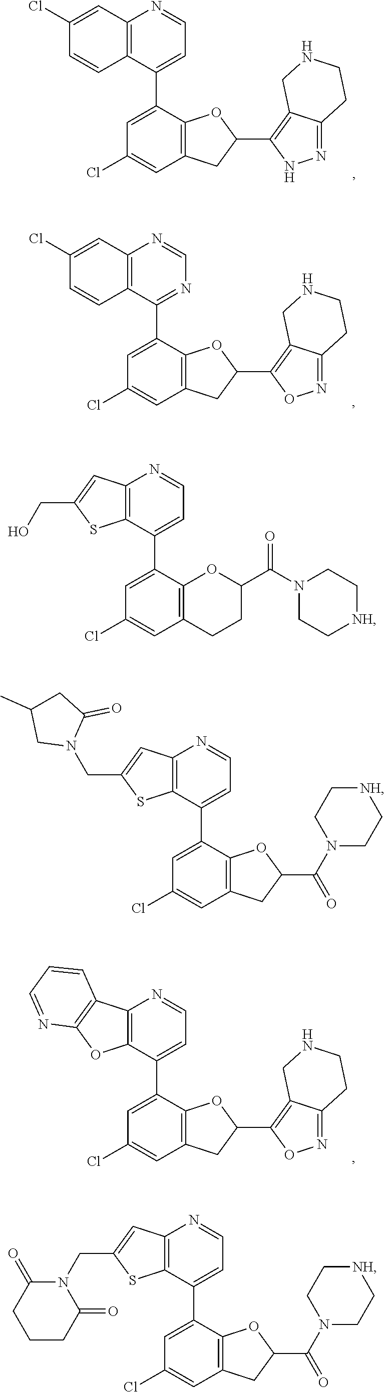 [SMILES: Clc1cc2c(c(-c3ccnc4cc(Cl)ccc34)c1)OC(c1[nH]nc3c1CNCC3)C2 ; Clc1cc2c(c(-c3ncnc4cc(Cl)ccc34)c1)OC(c1onc3c1CNCC3)C2 ; O=C(C1CCc2cc(Cl)cc(-c3ccnc4cc(CO)sc34)c2O1)N1CCNCC1 ; CC1CC(=O)N(Cc2cc3nccc(-c4cc(Cl)cc5c4OC(C(=O)N4CCNCC4)C5)c3s2)C1 ; Clc1cc2c(c(-c3ccnc4c3oc3ncccc34)c1)OC(c1onc3c1CNCC3)C2 ; O=C(C1Cc2cc(Cl)cc(-c3ccnc4cc(CN5C(=O)CCCC5=O)sc34)c2O1)N1CCNCC1]

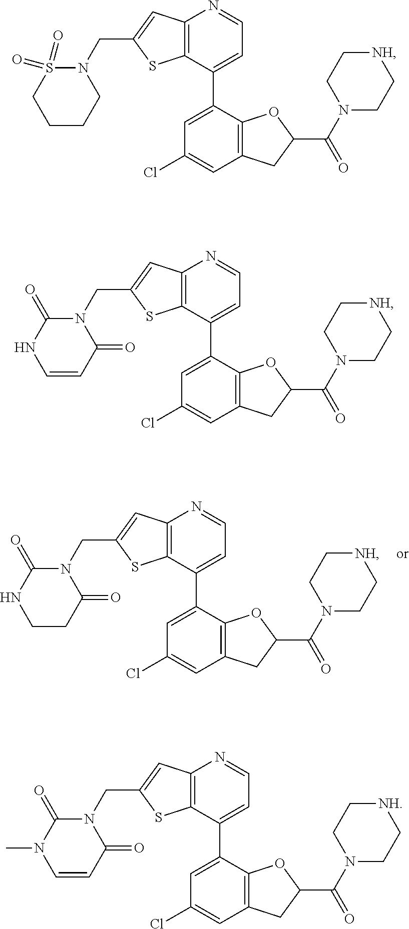 [SMILES: O=C(C1Cc2cc(Cl)cc(-c3ccnc4cc(CN5CCCCS5(=O)=O)sc34)c2O1)N1CCNCC1 ; O=C(C1Cc2cc(Cl)cc(-c3ccnc4cc(Cn5c(=O)cc[nH]c5=O)sc34)c2O1)N1CCNCC1 ; O=C(C1Cc2cc(Cl)cc(-c3ccnc4cc(CN5C(=O)CCNC5=O)sc34)c2O1)N1CCNCC1 ; Cn1ccc(=O)n(Cc2cc3nccc(-c4cc(Cl)cc5c4OC(C(=O)N4CCNCC4)C5)c3s2)c1=O]

In embodiments, the compound has the formula:

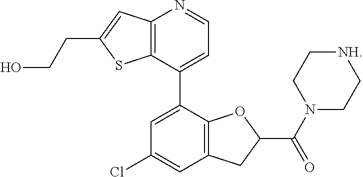 [SMILES: O=C(C1Cc2cc(Cl)cc(-c3ccnc4cc(CCO)sc34)c2O1)N1CCNCC1]

In embodiments, the compound has the formula:

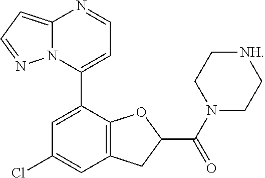 [SMILES: O=C(C1Cc2cc(Cl)cc(-c3ccnc4ccnn34)c2O1)N1CCNCC1]

In embodiments, the compound has the formula:

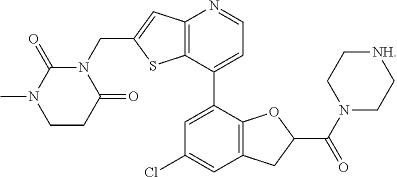 [SMILES: CN1CCC(=O)N(Cc2cc3nccc(-c4cc(Cl)cc5c4OC(C(=O)N4CCNCC4)C5)c3s2)C1=O]

In embodiments, the compound has the formula:

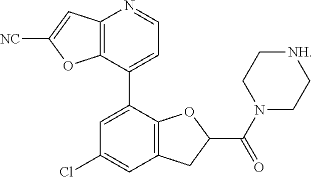 [SMILES: N#Cc1cc2nccc(-c3cc(Cl)cc4c3OC(C(=O)N3CCNCC3)C4)c2o1]

In embodiments, the compound has the formula:

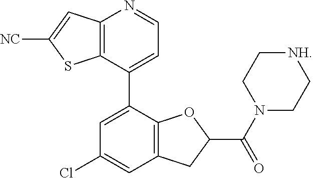 [SMILES: N#Cc1cc2nccc(-c3cc(Cl)cc4c3OC(C(=O)N3CCNCC3)C4)c2s1]

In embodiments, the compound has the formula:

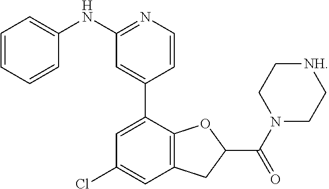 [SMILES: O=C(C1Cc2cc(Cl)cc(-c3ccnc(Nc4ccccc4)c3)c2O1)N1CCNCC1]

In embodiments, the compound has the formula:

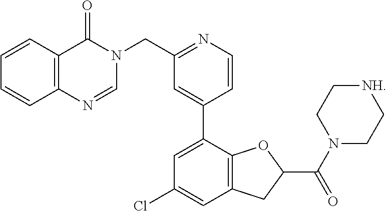 [SMILES: O=C(C1Cc2cc(Cl)cc(-c3ccnc(Cn4cnc5ccccc5c4=O)c3)c2O1)N1CCNCC1]

In embodiments, the compound has the formula:

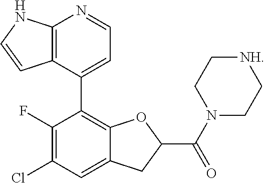 [SMILES: O=C(C1Cc2cc(Cl)c(F)c(-c3ccnc4[nH]ccc34)c2O1)N1CCNCC1]

In embodiments, the compound has the formula:

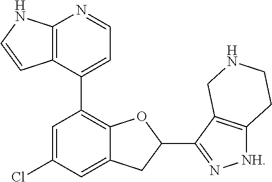 [SMILES: Clc1cc2c(c(-c3ccnc4[nH]ccc34)c1)OC(c1n[nH]c3c1CNCC3)C2]

In embodiments, the compound has the formula:

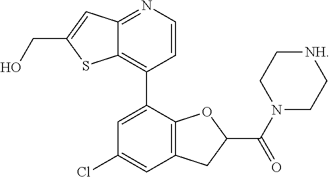 [SMILES: O=C(C1Cc2cc(Cl)cc(-c3ccnc4cc(CO)sc34)c2O1)N1CCNCC1]

In embodiments, the compound has the formula:

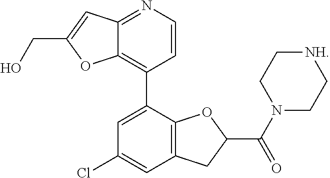 [SMILES: O=C(C1Cc2cc(Cl)cc(-c3ccnc4cc(CO)oc34)c2O1)N1CCNCC1]

In embodiments, the compound has the formula:

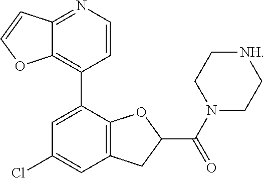 [SMILES: O=C(C1Cc2cc(Cl)cc(-c3ccnc4ccoc34)c2O1)N1CCNCC1]

In embodiments, the compound has the formula:

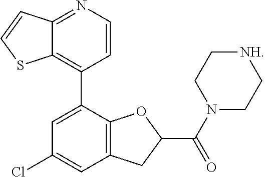 [SMILES: O=C(C1Cc2cc(Cl)cc(-c3ccnc4ccsc34)c2O1)N1CCNCC1]

In embodiments, the compound has the formula:

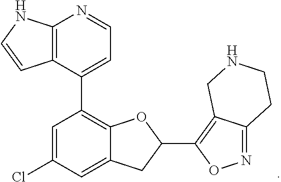 [SMILES: Clc1cc2c(c(-c3ccnc4[nH]ccc34)c1)OC(c1onc3c1CNCC3)C2]

In embodiments, the compound has the formula:

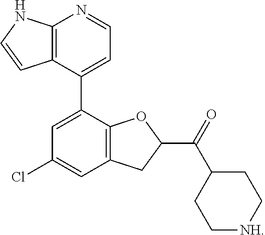 [SMILES: O=C(C1CCNCC1)C1Cc2cc(Cl)cc(-c3ccnc4[nH]ccc34)c2O1]

In embodiments, the compound has the formula:

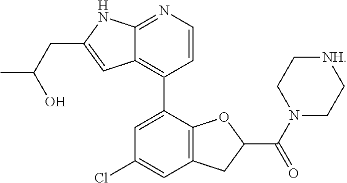 [SMILES: CC(O)Cc1cc2c(-c3cc(Cl)cc4c3OC(C(=O)N3CCNCC3)C4)ccnc2[nH]1]

In embodiments, the compound has the formula:

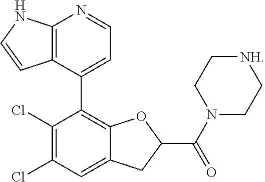 [SMILES: O=C(C1Cc2cc(Cl)c(Cl)c(-c3ccnc4[nH]ccc34)c2O1)N1CCNCC1]

In embodiments, the compound has the formula:

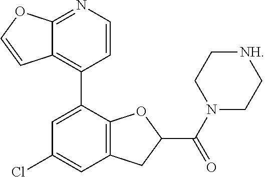 [SMILES: O=C(C1Cc2cc(Cl)cc(-c3ccnc4occc34)c2O1)N1CCNCC1]

In embodiments, the compound has the formula:

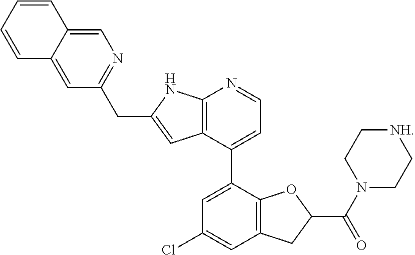 [SMILES: O=C(C1Cc2cc(Cl)cc(-c3ccnc4[nH]c(Cc5cc6ccccc6cn5)cc34)c2O1)N1CCNCC1]

In embodiments, the compound has the formula:

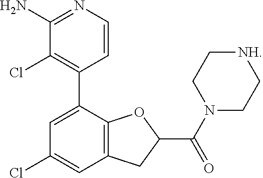 [SMILES: Nc1nccc(-c2cc(Cl)cc3c2OC(C(=O)N2CCNCC2)C3)c1Cl]

In embodiments, the compound has the formula:

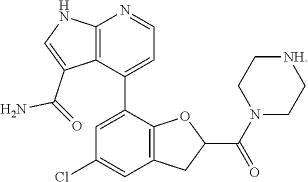 [SMILES: NC(=O)c1c[nH]c2nccc(-c3cc(Cl)cc4c3OC(C(=O)N3CCNCC3)C4)c12]

In embodiments, the compound has the formula:

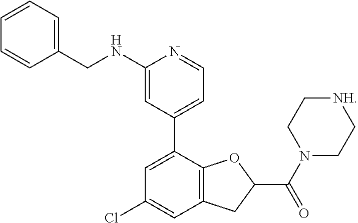 [SMILES: O=C(C1Cc2cc(Cl)cc(-c3ccnc(NCc4ccccc4)c3)c2O1)N1CCNCC1]

In embodiments, the compound has the formula:

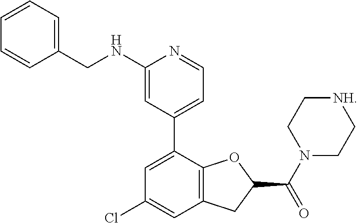

In embodiments, the compound has the formula:

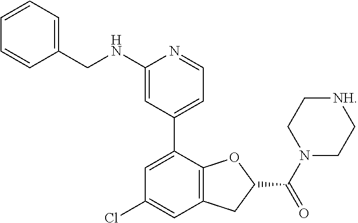

In embodiments, the compound has the formula:

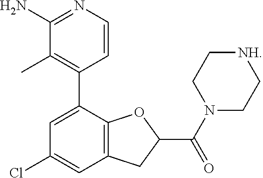 [SMILES: Cc1c(-c2cc(Cl)cc3c2OC(C(=O)N2CCNCC2)C3)ccnc1N]

In embodiments, the compound has the formula:

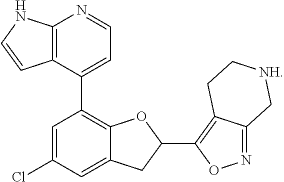 [SMILES: Clc1cc2c(c(-c3ccnc4[nH]ccc34)c1)OC(c1onc3c1CCNC3)C2]

In embodiments, the compound has the formula:

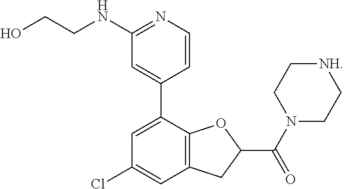 [SMILES: O=C(C1Cc2cc(Cl)cc(-c3ccnc(NCCO)c3)c2O1)N1CCNCC1]

In embodiments, the compound has the formula:

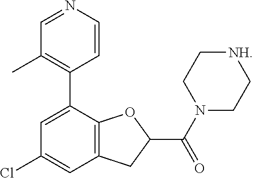 [SMILES: Cc1cnccc1-c1cc(Cl)cc2c1OC(C(=O)N1CCNCC1)C2]

In embodiments, the compound has the formula:

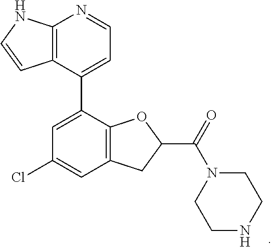 [SMILES: O=C(C1Cc2cc(Cl)cc(-c3ccnc4[nH]ccc34)c2O1)N1CCNCC1]

In embodiments, the compound has the formula:

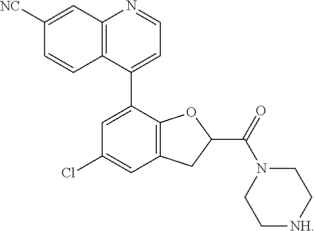 [SMILES: N#Cc1ccc2c(-c3cc(Cl)cc4c3OC(C(=O)N3CCNCC3)C4)ccnc2c1]

In embodiments, the compound has the formula:

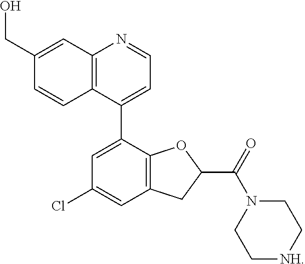 [SMILES: O=C(C1Cc2cc(Cl)cc(-c3ccnc4cc(CO)ccc34)c2O1)N1CCNCC1]

In embodiments, the compound has the formula:

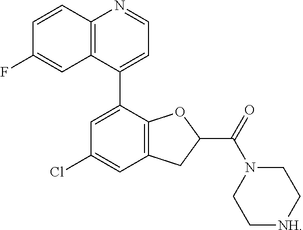 [SMILES: O=C(C1Cc2cc(Cl)cc(-c3ccnc4ccc(F)cc34)c2O1)N1CCNCC1]

In embodiments, the compound has the formula:

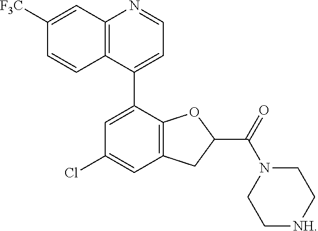 [SMILES: O=C(C1Cc2cc(Cl)cc(-c3ccnc4cc(C(F)(F)F)ccc34)c2O1)N1CCNCC1]

In embodiments, the compound has the formula:

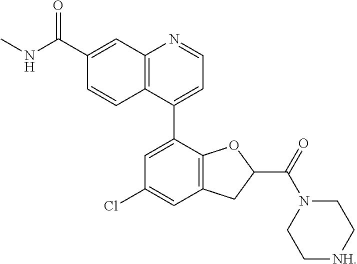 [SMILES: CNC(=O)c1ccc2c(-c3cc(Cl)cc4c3OC(C(=O)N3CCNCC3)C4)ccnc2c1]

In embodiments, the compound has the formula:

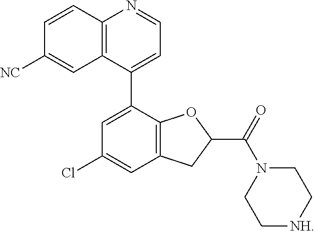 [SMILES: N#Cc1ccc2nccc(-c3cc(Cl)cc4c3OC(C(=O)N3CCNCC3)C4)c2c1]

In embodiments, the compound has the formula:

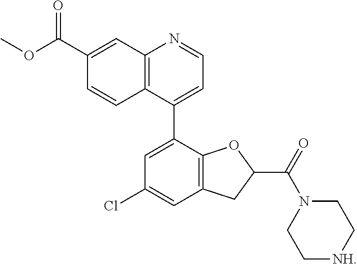 [SMILES: COC(=O)c1ccc2c(-c3cc(Cl)cc4c3OC(C(=O)N3CCNCC3)C4)ccnc2c1]

In embodiments, the compound has the formula:

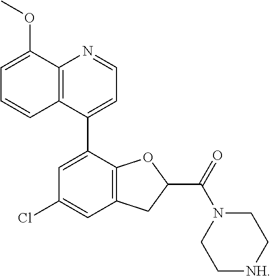 [SMILES: COc1cccc2c(-c3cc(Cl)cc4c3OC(C(=O)N3CCNCC3)C4)ccnc12]

In embodiments, the compound has the formula:

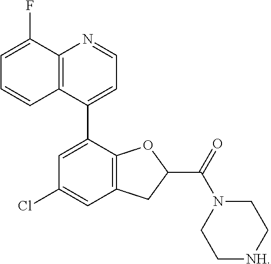 [SMILES: O=C(C1Cc2cc(Cl)cc(-c3ccnc4c(F)cccc34)c2O1)N1CCNCC1]

In embodiments, the compound has the formula:

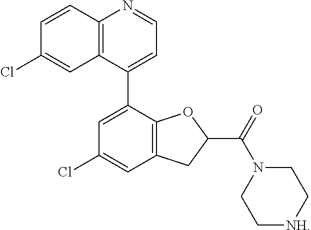 [SMILES: O=C(C1Cc2cc(Cl)cc(-c3ccnc4ccc(Cl)cc34)c2O1)N1CCNCC1]

In embodiments, the compound has the formula:

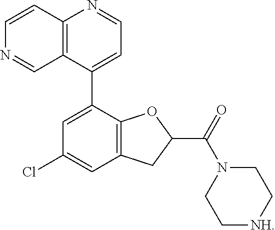 [SMILES: O=C(C1Cc2cc(Cl)cc(-c3ccnc4ccncc34)c2O1)N1CCNCC1]

In embodiments, the compound has the formula:

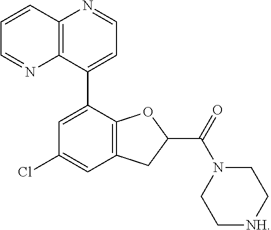 [SMILES: O=C(C1Cc2cc(Cl)cc(-c3ccnc4cccnc34)c2O1)N1CCNCC1]

In embodiments, the compound has the formula:

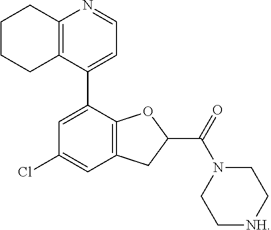 [SMILES: O=C(C1Cc2cc(Cl)cc(-c3ccnc4c3CCCC4)c2O1)N1CCNCC1]

In embodiments, the compound has the formula:

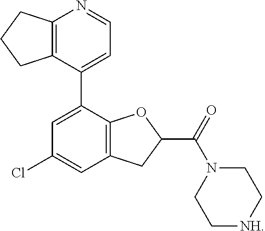 [SMILES: O=C(C1Cc2cc(Cl)cc(-c3ccnc4c3CCC4)c2O1)N1CCNCC1]

In embodiments, the compound has the formula:

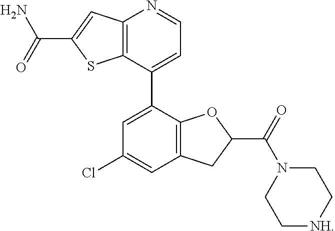 [SMILES: NC(=O)c1cc2nccc(-c3cc(Cl)cc4c3OC(C(=O)N3CCNCC3)C4)c2s1]

In embodiments, the compound has the formula:

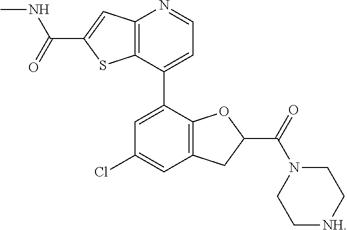 [SMILES: CNC(=O)c1cc2nccc(-c3cc(Cl)cc4c3OC(C(=O)N3CCNCC3)C4)c2s1]

In embodiments, the compound has the formula:

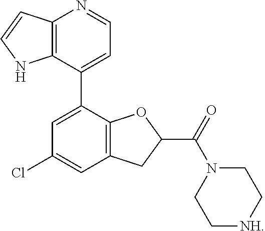 [SMILES: O=C(C1Cc2cc(Cl)cc(-c3ccnc4cc[nH]c34)c2O1)N1CCNCC1]

In embodiments, the compound has the formula:

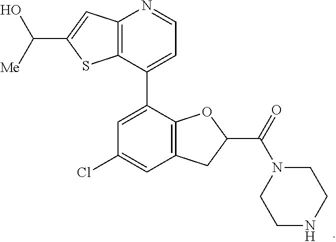 [SMILES: CC(O)c1cc2nccc(-c3cc(Cl)cc4c3OC(C(=O)N3CCNCC3)C4)c2s1]

In embodiments, the compound has the formula:

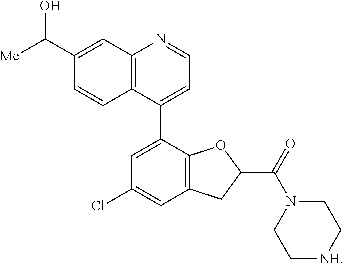 [SMILES: CC(O)c1ccc2c(-c3cc(Cl)cc4c3OC(C(=O)N3CCNCC3)C4)ccnc2c1]

In embodiments, the compound has the formula:

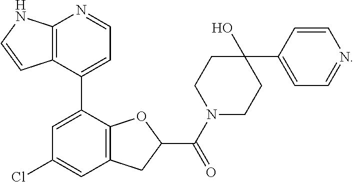 [SMILES: O=C(C1Cc2cc(Cl)cc(-c3ccnc4[nH]ccc34)c2O1)N1CCC(O)(c2ccncc2)CC1]

In embodiments, the compound has the formula:

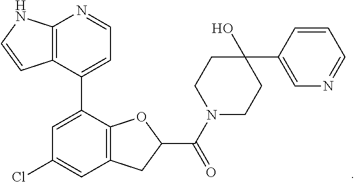 [SMILES: O=C(C1Cc2cc(Cl)cc(-c3ccnc4[nH]ccc34)c2O1)N1CCC(O)(c2cccnc2)CC1]

In embodiments, the compound has the formula:

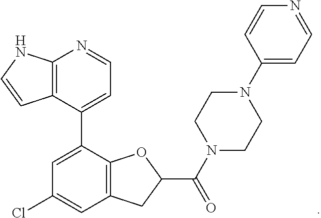 [SMILES: O=C(C1Cc2cc(Cl)cc(-c3ccnc4[nH]ccc34)c2O1)N1CCN(c2ccncc2)CC1]

In embodiments, the compound has the formula:

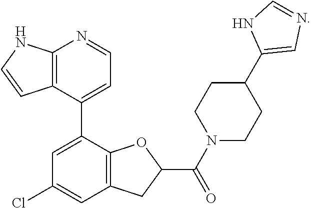 [SMILES: O=C(C1Cc2cc(Cl)cc(-c3ccnc4[nH]ccc34)c2O1)N1CCC(c2cnc[nH]2)CC1]

In embodiments, the compound has the formula:

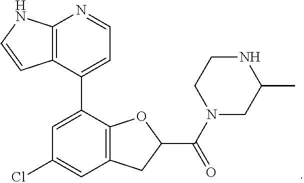 [SMILES: CC1CN(C(=O)C2Cc3cc(Cl)cc(-c4ccnc5[nH]ccc45)c3O2)CCN1]

In embodiments, the compound has the formula:

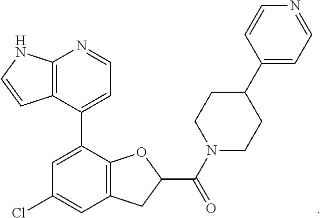 [SMILES: O=C(C1Cc2cc(Cl)cc(-c3ccnc4[nH]ccc34)c2O1)N1CCC(c2ccncc2)CC1]

In embodiments, the compound has the formula:

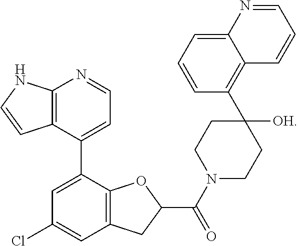 [SMILES: O=C(C1Cc2cc(Cl)cc(-c3ccnc4[nH]ccc34)c2O1)N1CCC(O)(c2cccc3ncccc23)CC1]

In embodiments, the compound has the formula:

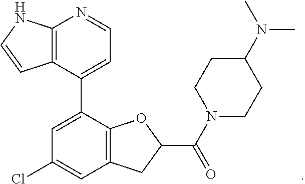 [SMILES: CN(C)C1CCN(C(=O)C2Cc3cc(Cl)cc(-c4ccnc5[nH]ccc45)c3O2)CC1]

In embodiments, the compound has the formula:

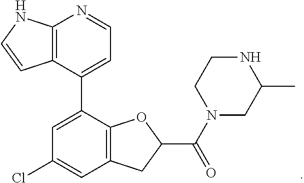 [SMILES: CC1CN(C(=O)C2Cc3cc(Cl)cc(-c4ccnc5[nH]ccc45)c3O2)CCN1]

In embodiments, the compound has the formula:

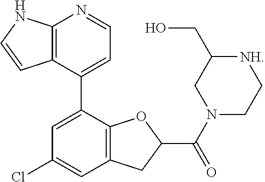 [SMILES: O=C(C1Cc2cc(Cl)cc(-c3ccnc4[nH]ccc34)c2O1)N1CCNC(CO)C1]

In embodiments, the compound has the formula:

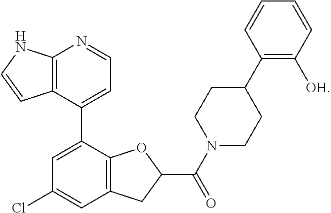 [SMILES: O=C(C1Cc2cc(Cl)cc(-c3ccnc4[nH]ccc34)c2O1)N1CCC(c2ccccc2O)CC1]

In embodiments, the compound has the formula:

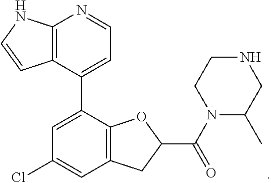 [SMILES: CC1CNCCN1C(=O)C1Cc2cc(Cl)cc(-c3ccnc4[nH]ccc34)c2O1]

In embodiments, the compound has the formula:

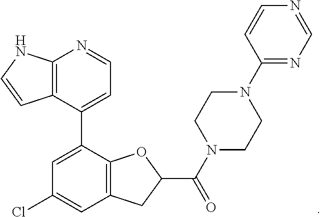 [SMILES: O=C(C1Cc2cc(Cl)cc(-c3ccnc4[nH]ccc34)c2O1)N1CCN(c2ccncn2)CC1]

In embodiments, the compound has the formula:

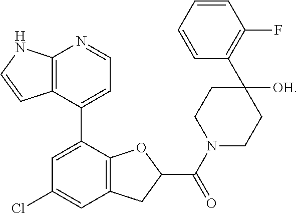 [SMILES: O=C(C1Cc2cc(Cl)cc(-c3ccnc4[nH]ccc34)c2O1)N1CCC(O)(c2ccccc2F)CC1]

In embodiments, the compound has the formula:

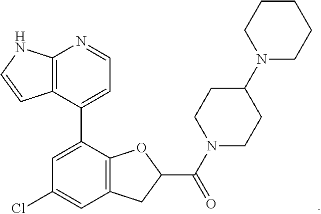 [SMILES: O=C(C1Cc2cc(Cl)cc(-c3ccnc4[nH]ccc34)c2O1)N1CCC(N2CCCCC2)CC1]

In embodiments, the compound has the formula:

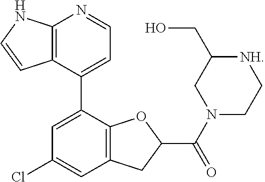 [SMILES: O=C(C1Cc2cc(Cl)cc(-c3ccnc4[nH]ccc34)c2O1)N1CCNC(CO)C1]

In embodiments, the compound has the formula:

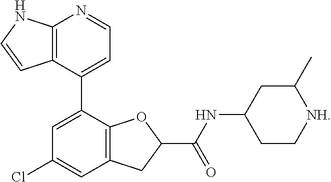 [SMILES: CC1CC(NC(=O)C2Cc3cc(Cl)cc(-c4ccnc5[nH]ccc45)c3O2)CCN1]

In embodiments, the compound has the formula:

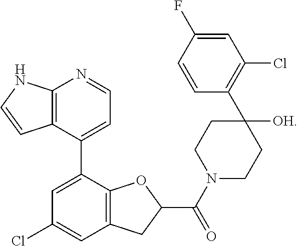 [SMILES: O=C(C1Cc2cc(Cl)cc(-c3ccnc4[nH]ccc34)c2O1)N1CCC(O)(c2ccc(F)cc2Cl)CC1]

In embodiments, the compound has the formula:

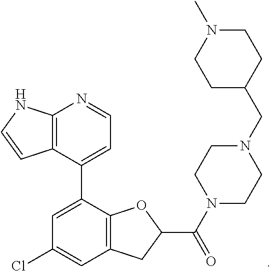 [SMILES: CN1CCC(CN2CCN(C(=O)C3Cc4cc(Cl)cc(-c5ccnc6[nH]ccc56)c4O3)CC2)CC1]

In embodiments, the compound has the formula:

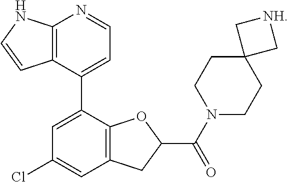 [SMILES: O=C(C1Cc2cc(Cl)cc(-c3ccnc4[nH]ccc34)c2O1)N1CCC2(CC1)CNC2]

In embodiments, the compound has the formula:

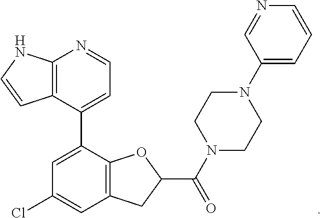 [SMILES: O=C(C1Cc2cc(Cl)cc(-c3ccnc4[nH]ccc34)c2O1)N1CCN(c2cccnc2)CC1]

In embodiments, the compound has the formula:

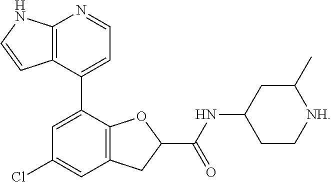 [SMILES: CC1CC(NC(=O)C2Cc3cc(Cl)cc(-c4ccnc5[nH]ccc45)c3O2)CCN1]

In embodiments, the compound has the formula:

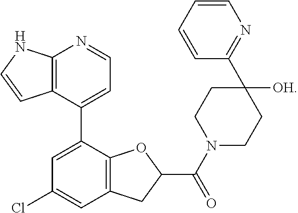 [SMILES: O=C(C1Cc2cc(Cl)cc(-c3ccnc4[nH]ccc34)c2O1)N1CCC(O)(c2ccccn2)CC1]

In embodiments, the compound has the formula:

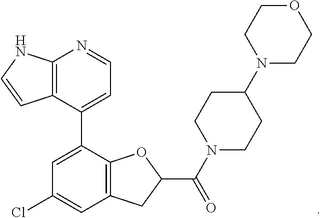 [SMILES: O=C(C1Cc2cc(Cl)cc(-c3ccnc4[nH]ccc34)c2O1)N1CCC(N2CCOCC2)CC1]

In embodiments, the compound has the formula:

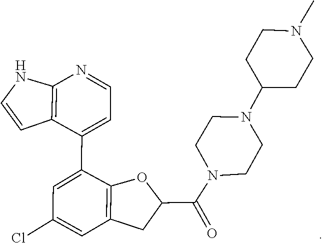 [SMILES: CN1CCC(N2CCN(C(=O)C3Cc4cc(Cl)cc(-c5ccnc6[nH]ccc56)c4O3)CC2)CC1]

In embodiments, the compound has the formula:

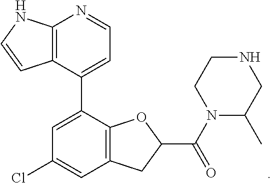 [SMILES: CC1CNCCN1C(=O)C1Cc2cc(Cl)cc(-c3ccnc4[nH]ccc34)c2O1]

In embodiments, the compound has the formula:

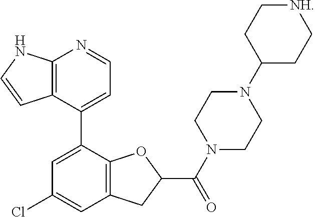 [SMILES: O=C(C1Cc2cc(Cl)cc(-c3ccnc4[nH]ccc34)c2O1)N1CCN(C2CCNCC2)CC1]

In embodiments, the compound has the formula:

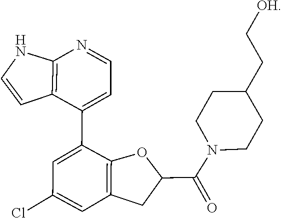 [SMILES: O=C(C1Cc2cc(Cl)cc(-c3ccnc4[nH]ccc34)c2O1)N1CCC(CCO)CC1]

In embodiments, the compound has the formula:

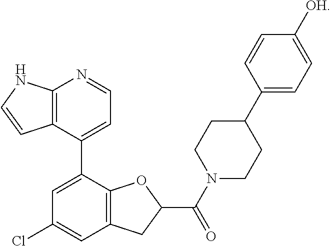 [SMILES: O=C(C1Cc2cc(Cl)cc(-c3ccnc4[nH]ccc34)c2O1)N1CCC(c2ccc(O)cc2)CC1]

In embodiments, the compound has the formula:

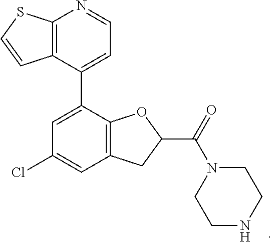 [SMILES: O=C(C1Cc2cc(Cl)cc(-c3ccnc4sccc34)c2O1)N1CCNCC1]

In embodiments, the compound has the formula:

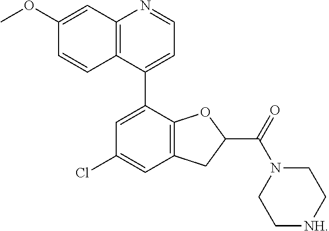 [SMILES: COc1ccc2c(-c3cc(Cl)cc4c3OC(C(=O)N3CCNCC3)C4)ccnc2c1]

In embodiments, the compound has the formula:

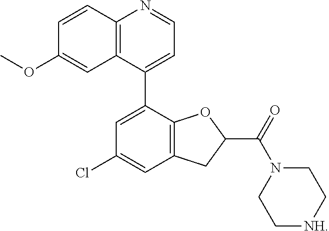 [SMILES: COc1ccc2nccc(-c3cc(Cl)cc4c3OC(C(=O)N3CCNCC3)C4)c2c1]

In embodiments, the compound has the formula:

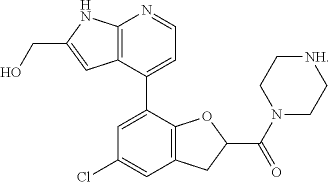 [SMILES: O=C(C1Cc2cc(Cl)cc(-c3ccnc4[nH]c(CO)cc34)c2O1)N1CCNCC1]

In embodiments, the compound has the formula:

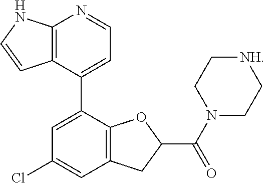 [SMILES: O=C(C1Cc2cc(Cl)cc(-c3ccnc4[nH]ccc34)c2O1)N1CCNCC1]

In embodiments, the compound has the formula:

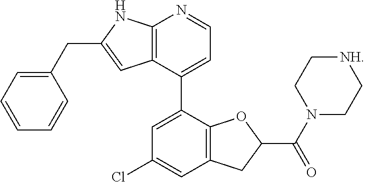 [SMILES: O=C(C1Cc2cc(Cl)cc(-c3ccnc4[nH]c(Cc5ccccc5)cc34)c2O1)N1CCNCC1]

In embodiments, the compound has the formula:

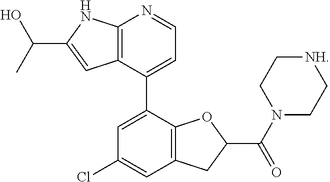 [SMILES: CC(O)c1cc2c(-c3cc(Cl)cc4c3OC(C(=O)N3CCNCC3)C4)ccnc2[nH]1]

In embodiments, the compound has the formula:

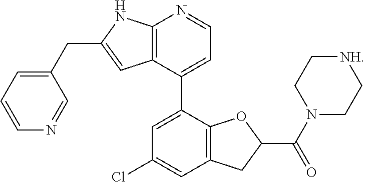 [SMILES: O=C(C1Cc2cc(Cl)cc(-c3ccnc4[nH]c(Cc5cccnc5)cc34)c2O1)N1CCNCC1]

In embodiments, the compound has the formula:

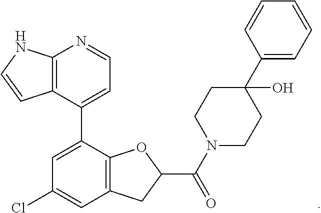 [SMILES: O=C(C1Cc2cc(Cl)cc(-c3ccnc4[nH]ccc34)c2O1)N1CCC(O)(c2ccccc2)CC1]

In embodiments, the compound has the formula:

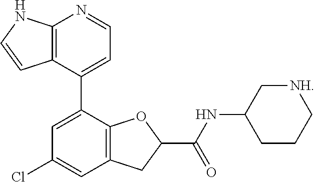 [SMILES: O=C(NC1CCCNC1)C1Cc2cc(Cl)cc(-c3ccnc4[nH]ccc34)c2O1]

In embodiments, the compound has the formula:

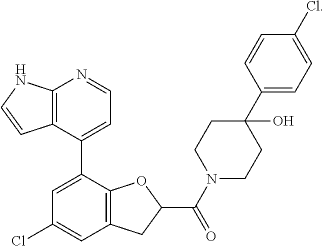 [SMILES: O=C(C1Cc2cc(Cl)cc(-c3ccnc4[nH]ccc34)c2O1)N1CCC(O)(c2ccc(Cl)cc2)CC1]

In embodiments, the compound has the formula:

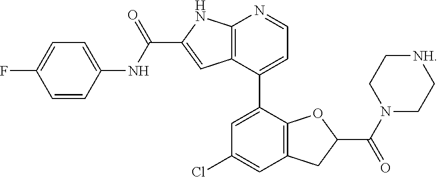 [SMILES: O=C(Nc1ccc(F)cc1)c1cc2c(-c3cc(Cl)cc4c3OC(C(=O)N3CCNCC3)C4)ccnc2[nH]1]

In embodiments, the compound has the formula:

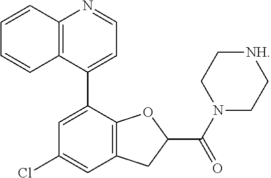 [SMILES: O=C(C1Cc2cc(Cl)cc(-c3ccnc4ccccc34)c2O1)N1CCNCC1]

In embodiments, the compound has the formula:

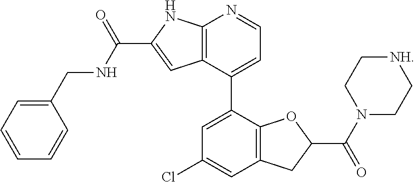 [SMILES: O=C(NCc1ccccc1)c1cc2c(-c3cc(Cl)cc4c3OC(C(=O)N3CCNCC3)C4)ccnc2[nH]1]

In embodiments, the compound has the formula:

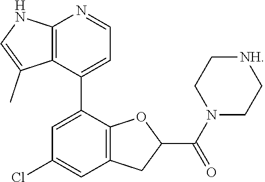 [SMILES: Cc1c[nH]c2nccc(-c3cc(Cl)cc4c3OC(C(=O)N3CCNCC3)C4)c12]

In embodiments, the compound has the formula:

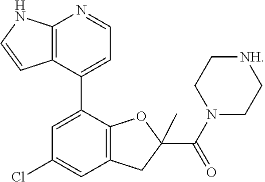 [SMILES: CC1(C(=O)N2CCNCC2)Cc2cc(Cl)cc(-c3ccnc4[nH]ccc34)c2O1]

In embodiments, the compound has the formula:

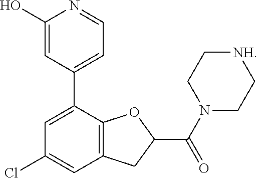 [SMILES: O=C(C1Cc2cc(Cl)cc(-c3ccnc(O)c3)c2O1)N1CCNCC1]

In embodiments, the compound has the formula:

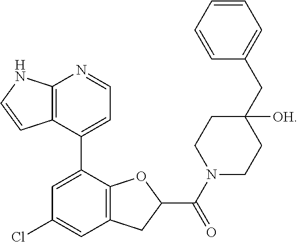 [SMILES: O=C(C1Cc2cc(Cl)cc(-c3ccnc4[nH]ccc34)c2O1)N1CCC(O)(Cc2ccccc2)CC1]

In embodiments, the compound has the formula:

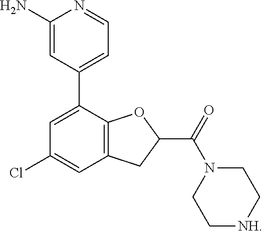 [SMILES: Nc1cc(-c2cc(Cl)cc3c2OC(C(=O)N2CCNCC2)C3)ccn1]

In embodiments, the compound has the formula:

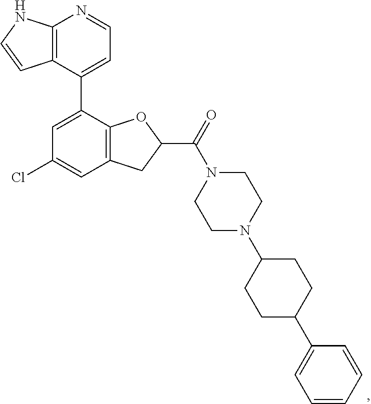 [SMILES: O=C(C1Cc2cc(Cl)cc(-c3ccnc4[nH]ccc34)c2O1)N1CCN(C2CCC(c3ccccc3)CC2)CC1]

In embodiments, the compound has the formula:

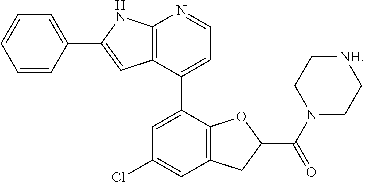 [SMILES: O=C(C1Cc2cc(Cl)cc(-c3ccnc4[nH]c(-c5ccccc5)cc34)c2O1)N1CCNCC1]

In embodiments, the compound has the formula:

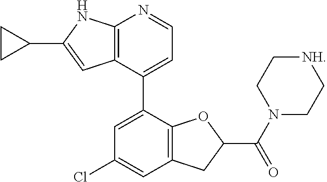 [SMILES: O=C(C1Cc2cc(Cl)cc(-c3ccnc4[nH]c(C5CC5)cc34)c2O1)N1CCNCC1]

In embodiments, the compound has the formula:

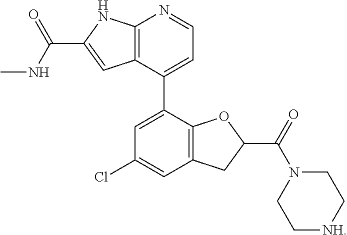 [SMILES: CNC(=O)c1cc2c(-c3cc(Cl)cc4c3OC(C(=O)N3CCNCC3)C4)ccnc2[nH]1]

In embodiments, the compound has the formula:

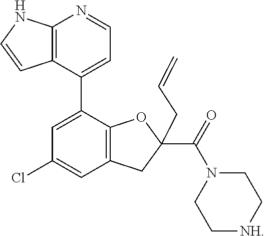 [SMILES: C=CCC1(C(=O)N2CCNCC2)Cc2cc(Cl)cc(-c3ccnc4[nH]ccc34)c2O1]

In embodiments, the compound has the formula:

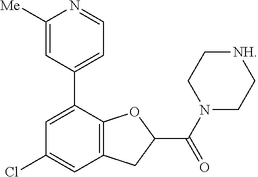 [SMILES: Cc1cc(-c2cc(Cl)cc3c2OC(C(=O)N2CCNCC2)C3)ccn1]

In embodiments, the compound has the formula:

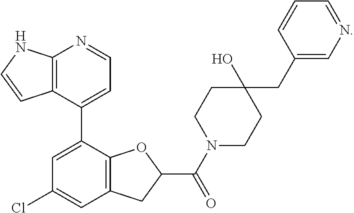 [SMILES: O=C(C1Cc2cc(Cl)cc(-c3ccnc4[nH]ccc34)c2O1)N1CCC(O)(Cc2cccnc2)CC1]

In embodiments, the compound has the formula:

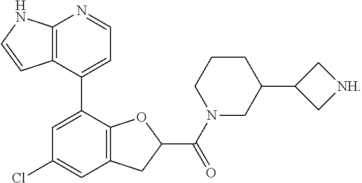 [SMILES: O=C(C1Cc2cc(Cl)cc(-c3ccnc4[nH]ccc34)c2O1)N1CCCC(C2CNC2)C1]

In embodiments, the compound has the formula:

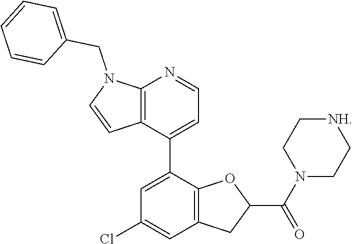 [SMILES: O=C(C1Cc2cc(Cl)cc(-c3ccnc4c3ccn4Cc3ccccc3)c2O1)N1CCNCC1]

In embodiments, the compound has the formula:

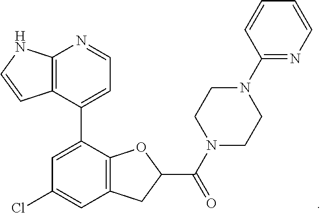 [SMILES: O=C(C1Cc2cc(Cl)cc(-c3ccnc4[nH]ccc34)c2O1)N1CCN(c2ccccn2)CC1]

In embodiments, the compound has the formula:

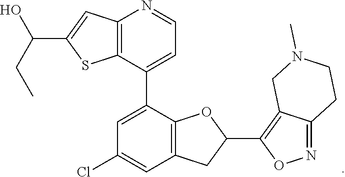 [SMILES: CCC(O)c1cc2nccc(-c3cc(Cl)cc4c3OC(c3onc5c3CN(C)CC5)C4)c2s1]

In embodiments, the compound has the formula:

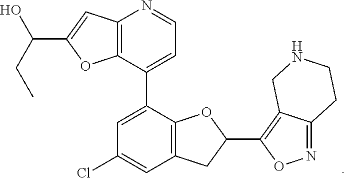 [SMILES: CCC(O)c1cc2nccc(-c3cc(Cl)cc4c3OC(c3onc5c3CNCC5)C4)c2o1]

In embodiments, the compound has the formula:

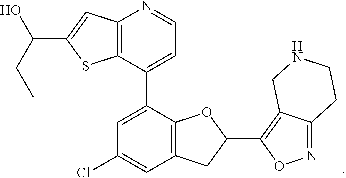 [SMILES: CCC(O)c1cc2nccc(-c3cc(Cl)cc4c3OC(c3onc5c3CNCC5)C4)c2s1]

In embodiments, the compound has the formula:

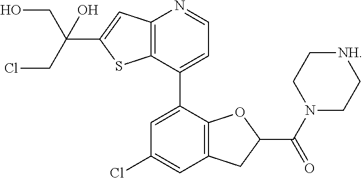 [SMILES: O=C(C1Cc2cc(Cl)cc(-c3ccnc4cc(C(O)(CO)CCl)sc34)c2O1)N1CCNCC1]

In embodiments, the compound has the formula:

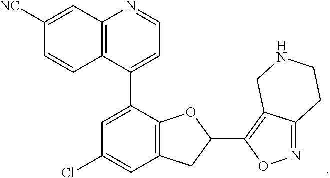 [SMILES: N#Cc1ccc2c(-c3cc(Cl)cc4c3OC(c3onc5c3CNCC5)C4)ccnc2c1]

In embodiments, the compound has the formula:

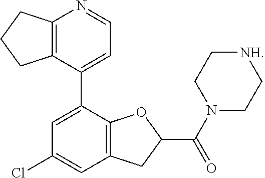 [SMILES: O=C(C1Cc2cc(Cl)cc(-c3ccnc4c3CCC4)c2O1)N1CCNCC1]

In embodiments, the compound has the formula:

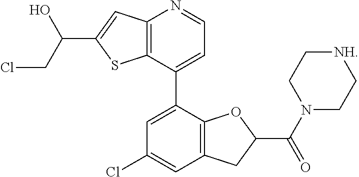 [SMILES: O=C(C1Cc2cc(Cl)cc(-c3ccnc4cc(C(O)CCl)sc34)c2O1)N1CCNCC1]

In embodiments, the compound has the formula:

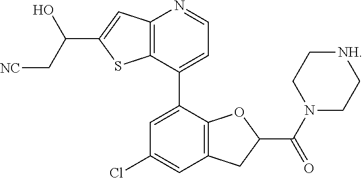 [SMILES: N#CCC(O)c1cc2nccc(-c3cc(Cl)cc4c3OC(C(=O)N3CCNCC3)C4)c2s1]

In embodiments, the compound has the formula:

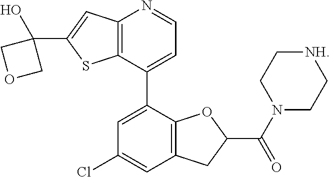 [SMILES: O=C(C1Cc2cc(Cl)cc(-c3ccnc4cc(C5(O)COC5)sc34)c2O1)N1CCNCC1]

In embodiments, the compound has the formula:

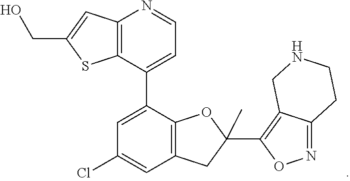 [SMILES: CC1(c2onc3c2CNCC3)Cc2cc(Cl)cc(-c3ccnc4cc(CO)sc34)c2O1]

In embodiments, the compound has the formula:

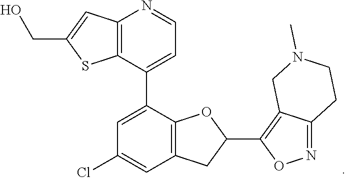 [SMILES: CN1CCc2noc(C3Cc4cc(Cl)cc(-c5ccnc6cc(CO)sc56)c4O3)c2C1]

In embodiments, the compound has the formula:

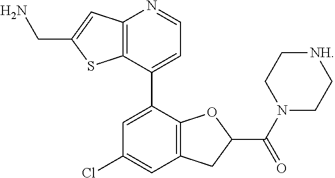 [SMILES: NCc1cc2nccc(-c3cc(Cl)cc4c3OC(C(=O)N3CCNCC3)C4)c2s1]

In embodiments, the compound has the formula:

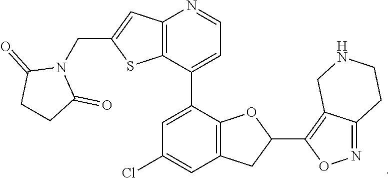 [SMILES: O=C1CCC(=O)N1Cc1cc2nccc(-c3cc(Cl)cc4c3OC(c3onc5c3CNCC5)C4)c2s1]

In embodiments, the compound has the formula:

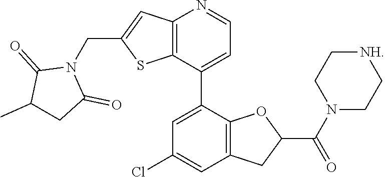 [SMILES: CC1CC(=O)N(Cc2cc3nccc(-c4cc(Cl)cc5c4OC(C(=O)N4CCNCC4)C5)c3s2)C1=O]

In embodiments, the compound has the formula:

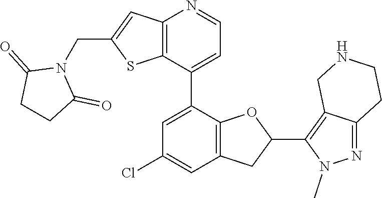 [SMILES: Cn1nc2c(c1C1Cc3cc(Cl)cc(-c4ccnc5cc(CN6C(=O)CCC6=O)sc45)c3O1)CNCC2]

In embodiments, the compound has the formula:

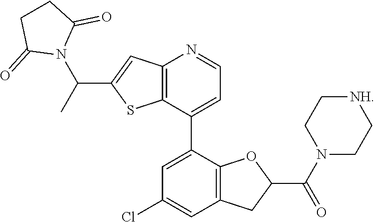 [SMILES: CC(c1cc2nccc(-c3cc(Cl)cc4c3OC(C(=O)N3CCNCC3)C4)c2s1)N1C(=O)CCC1=O]

In embodiments, the compound has the formula:

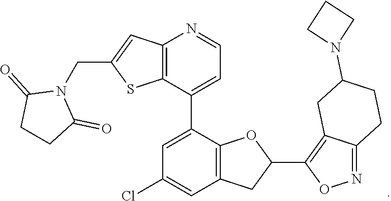 [SMILES: O=C1CCC(=O)N1Cc1cc2nccc(-c3cc(Cl)cc4c3OC(c3onc5c3CC(N3CCC3)CC5)C4)c2s1]

In embodiments, the compound has the formula:

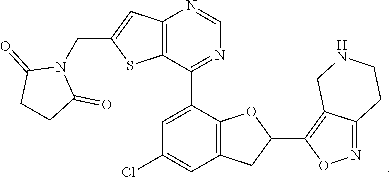 [SMILES: O=C1CCC(=O)N1Cc1cc2ncnc(-c3cc(Cl)cc4c3OC(c3onc5c3CNCC5)C4)c2s1]

In embodiments, the compound has the formula:

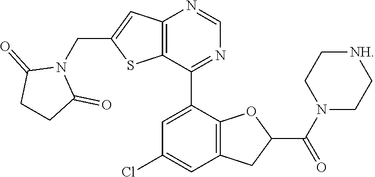 [SMILES: O=C(C1Cc2cc(Cl)cc(-c3ncnc4cc(CN5C(=O)CCC5=O)sc34)c2O1)N1CCNCC1]

In embodiments, the compound has the formula:

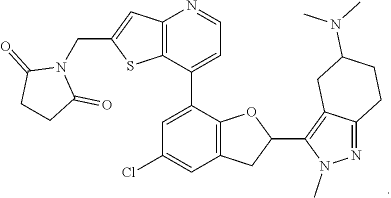 [SMILES: CN(C)C1CCc2nn(C)c(C3Cc4cc(Cl)cc(-c5ccnc6cc(CN7C(=O)CCC7=O)sc56)c4O3)c2C1]

In embodiments, the compound has the formula:

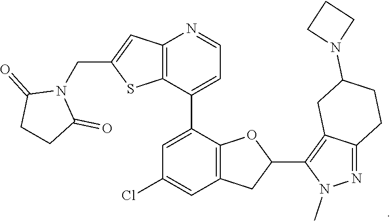 [SMILES: Cn1nc2c(c1C1Cc3cc(Cl)cc(-c4ccnc5cc(CN6C(=O)CCC6=O)sc45)c3O1)CC(N1CCC1)CC2]

In embodiments, the compound has the formula:

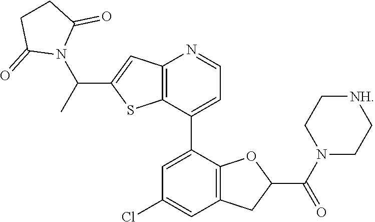 [SMILES: CC(c1cc2nccc(-c3cc(Cl)cc4c3OC(C(=O)N3CCNCC3)C4)c2s1)N1C(=O)CCC1=O]

In embodiments, the compound has the formula:

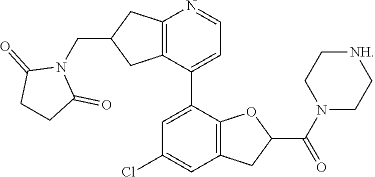 [SMILES: O=C(C1Cc2cc(Cl)cc(-c3ccnc4c3CC(CN3C(=O)CCC3=O)C4)c2O1)N1CCNCC1]

In embodiments, the compound has the formula:

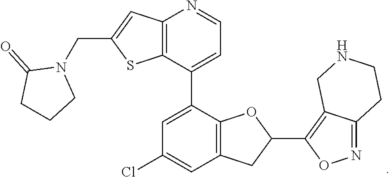 [SMILES: O=C1CCCN1Cc1cc2nccc(-c3cc(Cl)cc4c3OC(c3onc5c3CNCC5)C4)c2s1]

In embodiments, the compound has the formula:

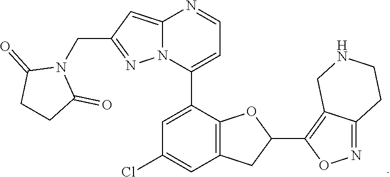 [SMILES: O=C1CCC(=O)N1Cc1cc2nccc(-c3cc(Cl)cc4c3OC(c3onc5c3CNCC5)C4)n2n1]

In embodiments, the compound has the formula:

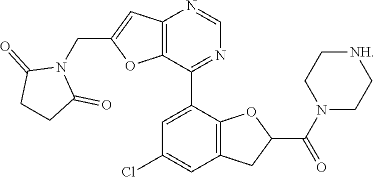 [SMILES: O=C(C1Cc2cc(Cl)cc(-c3ncnc4cc(CN5C(=O)CCC5=O)oc34)c2O1)N1CCNCC1]

In embodiments, the compound has the formula:

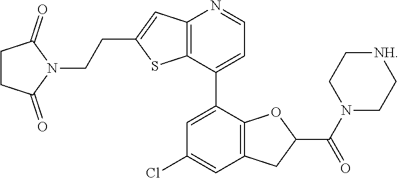 [SMILES: O=C(C1Cc2cc(Cl)cc(-c3ccnc4cc(CCN5C(=O)CCC5=O)sc34)c2O1)N1CCNCC1]

In embodiments, the compound has the formula:

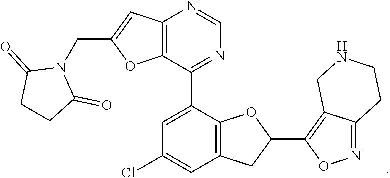 [SMILES: O=C1CCC(=O)N1Cc1cc2ncnc(-c3cc(Cl)cc4c3OC(c3onc5c3CNCC5)C4)c2o1]

In embodiments, the compound has the formula:

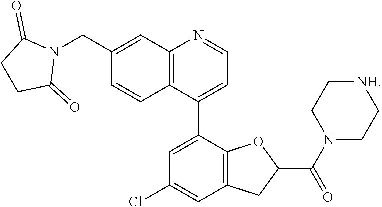 [SMILES: O=C(C1Cc2cc(Cl)cc(-c3ccnc4cc(CN5C(=O)CCC5=O)ccc34)c2O1)N1CCNCC1]

In embodiments, the compound has the formula:

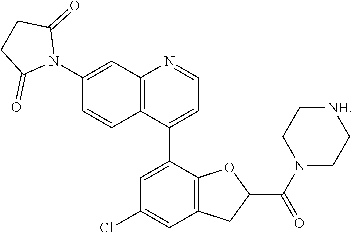 [SMILES: O=C(C1Cc2cc(Cl)cc(-c3ccnc4cc(N5C(=O)CCC5=O)ccc34)c2O1)N1CCNCC1]

In embodiments, the compound has the formula:

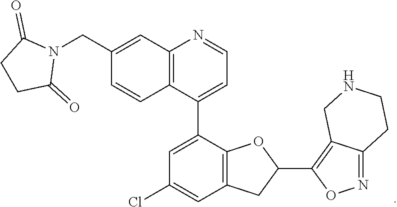 [SMILES: O=C1CCC(=O)N1Cc1ccc2c(-c3cc(Cl)cc4c3OC(c3onc5c3CNCC5)C4)ccnc2c1]

In embodiments, the compound has the formula:

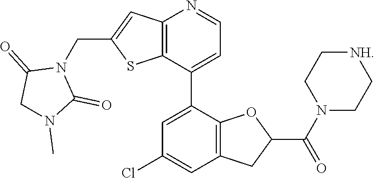 [SMILES: CN1CC(=O)N(Cc2cc3nccc(-c4cc(Cl)cc5c4OC(C(=O)N4CCNCC4)C5)c3s2)C1=O]

In embodiments, the compound has the formula:

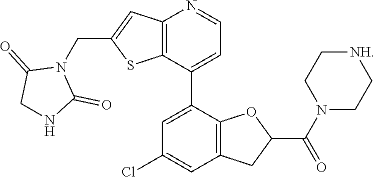 [SMILES: O=C(C1Cc2cc(Cl)cc(-c3ccnc4cc(CN5C(=O)CNC5=O)sc34)c2O1)N1CCNCC1]

In embodiments, the compound has the formula:

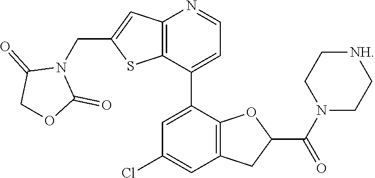 [SMILES: O=C(C1Cc2cc(Cl)cc(-c3ccnc4cc(CN5C(=O)COC5=O)sc34)c2O1)N1CCNCC1]

In embodiments, the compound has the formula:

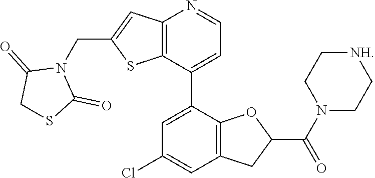 [SMILES: O=C(C1Cc2cc(Cl)cc(-c3ccnc4cc(CN5C(=O)CSC5=O)sc34)c2O1)N1CCNCC1]

In embodiments, the compound has the formula:

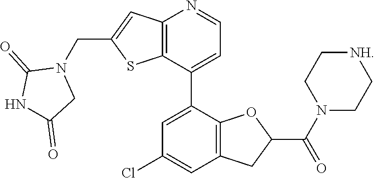 [SMILES: O=C1CN(Cc2cc3nccc(-c4cc(Cl)cc5c4OC(C(=O)N4CCNCC4)C5)c3s2)C(=O)N1]

In embodiments, the compound has the formula:

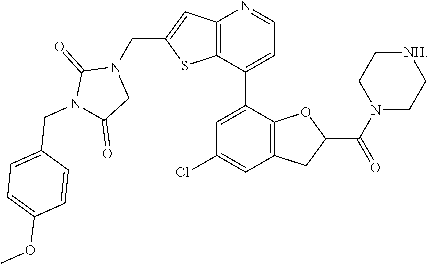 [SMILES: COc1ccc(CN2C(=O)CN(Cc3cc4nccc(-c5cc(Cl)cc6c5OC(C(=O)N5CCNCC5)C6)c4s3)C2=O)cc1]

In embodiments, the compound has the formula:

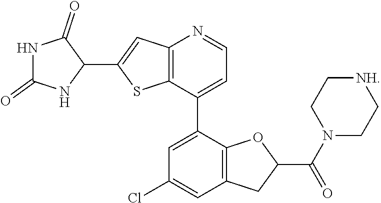 [SMILES: O=C1NC(=O)C(c2cc3nccc(-c4cc(Cl)cc5c4OC(C(=O)N4CCNCC4)C5)c3s2)N1]

In embodiments, the compound has the formula:

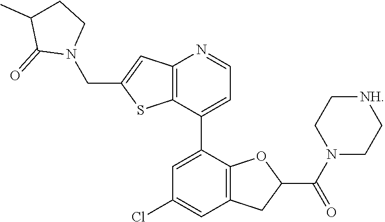 [SMILES: CC1CCN(Cc2cc3nccc(-c4cc(Cl)cc5c4OC(C(=O)N4CCNCC4)C5)c3s2)C1=O]

In embodiments, the compound has the formula:

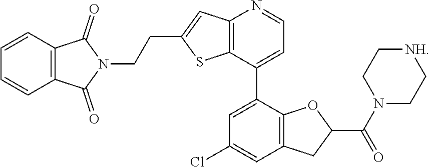 [SMILES: O=C(C1Cc2cc(Cl)cc(-c3ccnc4cc(CCN5C(=O)c6ccccc6C5=O)sc34)c2O1)N1CCNCC1]

In embodiments, the compound has the formula:

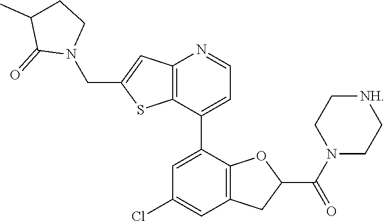 [SMILES: CC1CCN(Cc2cc3nccc(-c4cc(Cl)cc5c4OC(C(=O)N4CCNCC4)C5)c3s2)C1=O]

In embodiments, the compound has the formula:

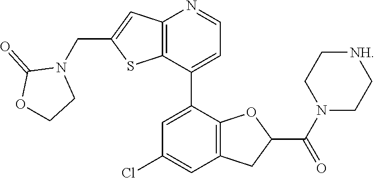 [SMILES: O=C1OCCN1Cc1cc2nccc(-c3cc(Cl)cc4c3OC(C(=O)N3CCNCC3)C4)c2s1]

In embodiments, the compound has the formula:

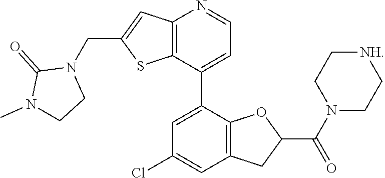 [SMILES: CN1CCN(Cc2cc3nccc(-c4cc(Cl)cc5c4OC(C(=O)N4CCNCC4)C5)c3s2)C1=O]

In embodiments, the compound has the formula:

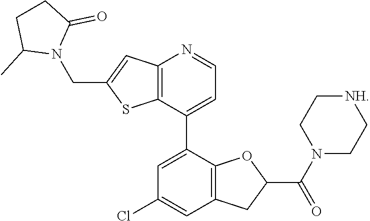 [SMILES: CC1CCC(=O)N1Cc1cc2nccc(-c3cc(Cl)cc4c3OC(C(=O)N3CCNCC3)C4)c2s1]

In embodiments, the compound has the formula:

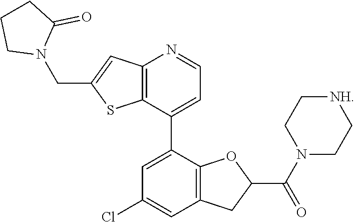 [SMILES: O=C1CCCN1Cc1cc2nccc(-c3cc(Cl)cc4c3OC(C(=O)N3CCNCC3)C4)c2s1]

In embodiments, the compound has the formula:

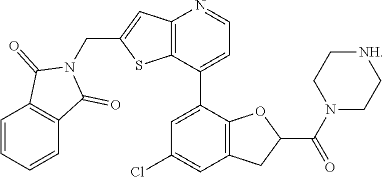 [SMILES: O=C(C1Cc2cc(Cl)cc(-c3ccnc4cc(CN5C(=O)c6ccccc6C5=O)sc34)c2O1)N1CCNCC1]

In embodiments, the compound has the formula:

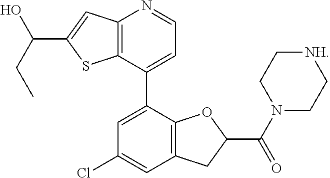 [SMILES: CCC(O)c1cc2nccc(-c3cc(Cl)cc4c3OC(C(=O)N3CCNCC3)C4)c2s1]

In embodiments, the compound has the formula:

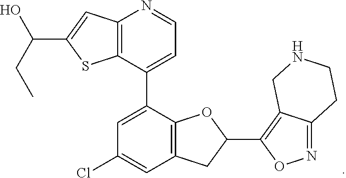 [SMILES: CCC(O)c1cc2nccc(-c3cc(Cl)cc4c3OC(c3onc5c3CNCC5)C4)c2s1]

In embodiments, the compound has the formula:

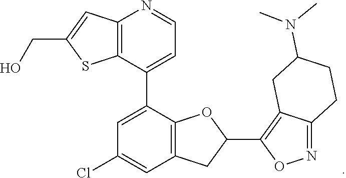 [SMILES: CN(C)C1CCc2noc(C3Cc4cc(Cl)cc(-c5ccnc6cc(CO)sc56)c4O3)c2C1]

In embodiments, the compound has the formula:

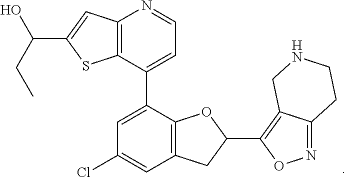 [SMILES: CCC(O)c1cc2nccc(-c3cc(Cl)cc4c3OC(c3onc5c3CNCC5)C4)c2s1]

In embodiments, the compound has the formula:

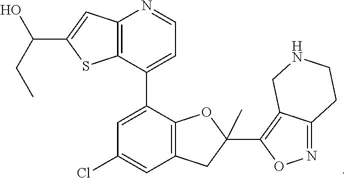 [SMILES: CCC(O)c1cc2nccc(-c3cc(Cl)cc4c3OC(C)(c3onc5c3CNCC5)C4)c2s1]

In embodiments, the compound has the formula:

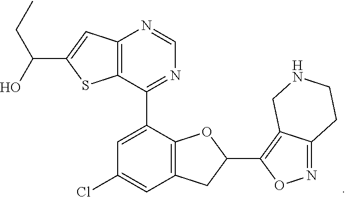 [SMILES: CCC(O)c1cc2ncnc(-c3cc(Cl)cc4c3OC(c3onc5c3CNCC5)C4)c2s1]

In embodiments, the compound has the formula:

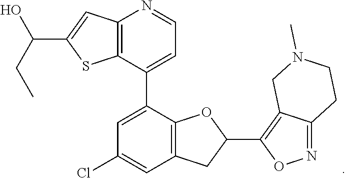 [SMILES: CCC(O)c1cc2nccc(-c3cc(Cl)cc4c3OC(c3onc5c3CN(C)CC5)C4)c2s1]

In embodiments, the compound has the formula:

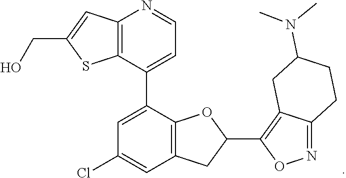 [SMILES: CN(C)C1CCc2noc(C3Cc4cc(Cl)cc(-c5ccnc6cc(CO)sc56)c4O3)c2C1]

In embodiments, the compound has the formula:

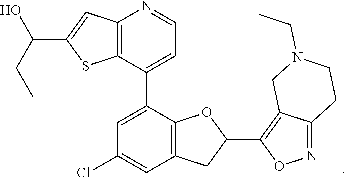 [SMILES: CCC(O)c1cc2nccc(-c3cc(Cl)cc4c3OC(c3onc5c3CN(CC)CC5)C4)c2s1]

In embodiments, the compound has the formula:

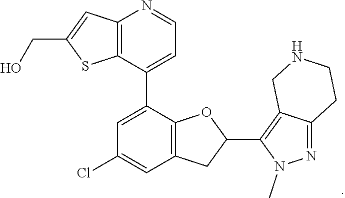 [SMILES: Cn1nc2c(c1C1Cc3cc(Cl)cc(-c4ccnc5cc(CO)sc45)c3O1)CNCC2]

In embodiments, the compound has the formula:

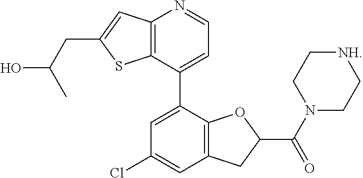 [SMILES: CC(O)Cc1cc2nccc(-c3cc(Cl)cc4c3OC(C(=O)N3CCNCC3)C4)c2s1]

In embodiments, the compound has the formula:

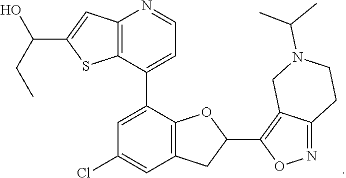 [SMILES: CCC(O)c1cc2nccc(-c3cc(Cl)cc4c3OC(c3onc5c3CN(C(C)C)CC5)C4)c2s1]

In embodiments, the compound has the formula:

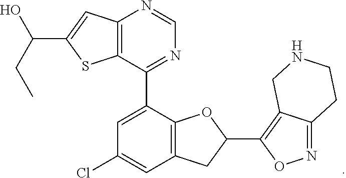 [SMILES: CCC(O)c1cc2ncnc(-c3cc(Cl)cc4c3OC(c3onc5c3CNCC5)C4)c2s1]

In embodiments, the compound has the formula:

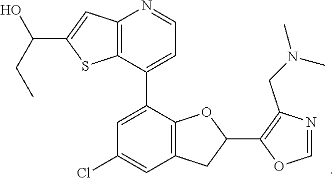 [SMILES: CCC(O)c1cc2nccc(-c3cc(Cl)cc4c3OC(c3ocnc3CN(C)C)C4)c2s1]

In embodiments, the compound has the formula:

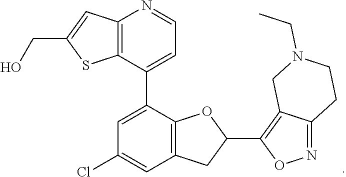 [SMILES: CCN1CCc2noc(C3Cc4cc(Cl)cc(-c5ccnc6cc(CO)sc56)c4O3)c2C1]

In embodiments, the compound has the formula:

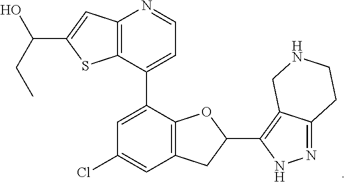 [SMILES: CCC(O)c1cc2nccc(-c3cc(Cl)cc4c3OC(c3[nH]nc5c3CNCC5)C4)c2s1]

In embodiments, the compound has the formula:

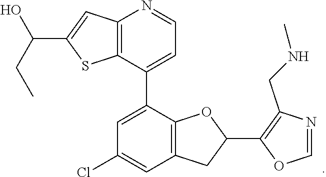 [SMILES: CCC(O)c1cc2nccc(-c3cc(Cl)cc4c3OC(c3ocnc3CNC)C4)c2s1]

In embodiments, the compound has the formula:

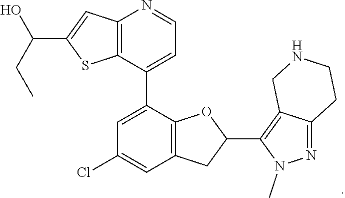 [SMILES: CCC(O)c1cc2nccc(-c3cc(Cl)cc4c3OC(c3c5c(nn3C)CCNC5)C4)c2s1]

In embodiments, the compound has the formula:

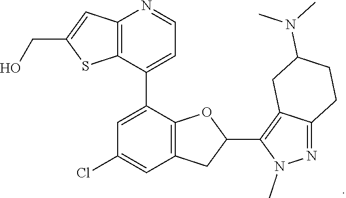 [SMILES: CN(C)C1CCc2nn(C)c(C3Cc4cc(Cl)cc(-c5ccnc6cc(CO)sc56)c4O3)c2C1]

In embodiments, the compound has the formula:

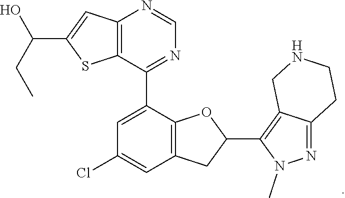 [SMILES: CCC(O)c1cc2ncnc(-c3cc(Cl)cc4c3OC(c3c5c(nn3C)CCNC5)C4)c2s1]

In embodiments, the compound has the formula:

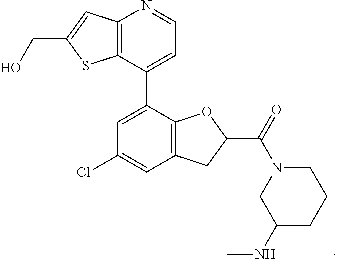 [SMILES: CNC1CCCN(C(=O)C2Cc3cc(Cl)cc(-c4ccnc5cc(CO)sc45)c3O2)C1]

In embodiments, the compound has the formula:

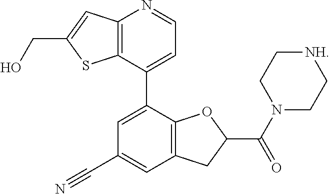 [SMILES: N#Cc1cc2c(c(-c3ccnc4cc(CO)sc34)c1)OC(C(=O)N1CCNCC1)C2]

In embodiments, the compound has the formula:

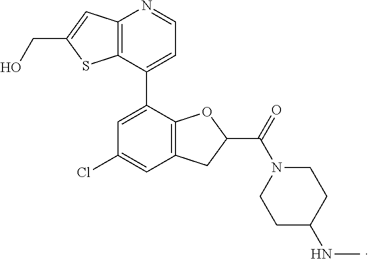 [SMILES: CNC1CCN(C(=O)C2Cc3cc(Cl)cc(-c4ccnc5cc(CO)sc45)c3O2)CC1]

In embodiments, the compound has the formula:

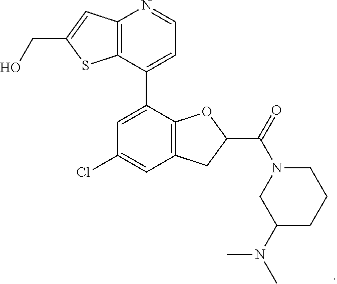 [SMILES: CN(C)C1CCCN(C(=O)C2Cc3cc(Cl)cc(-c4ccnc5cc(CO)sc45)c3O2)C1]

In embodiments, the compound has the formula:

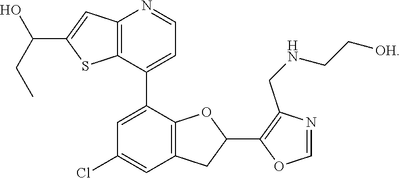 [SMILES: CCC(O)c1cc2nccc(-c3cc(Cl)cc4c3OC(c3ocnc3CNCCO)C4)c2s1]

In embodiments, the compound has the formula:

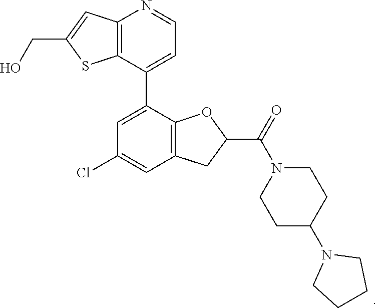 [SMILES: O=C(C1Cc2cc(Cl)cc(-c3ccnc4cc(CO)sc34)c2O1)N1CCC(N2CCCC2)CC1]

In embodiments, the compound has the formula:

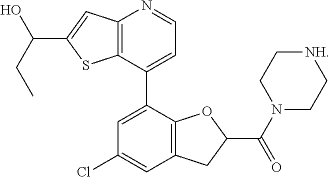 [SMILES: CCC(O)c1cc2nccc(-c3cc(Cl)cc4c3OC(C(=O)N3CCNCC3)C4)c2s1]

In embodiments, the compound has the formula:

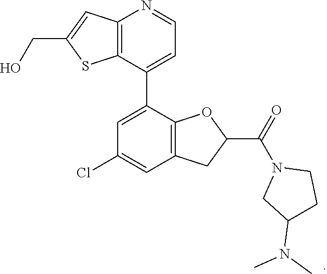 [SMILES: CN(C)C1CCN(C(=O)C2Cc3cc(Cl)cc(-c4ccnc5cc(CO)sc45)c3O2)C1]

In embodiments, the compound has the formula:

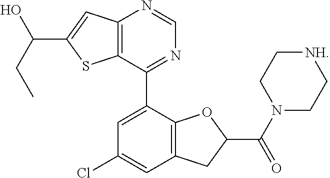 [SMILES: CCC(O)c1cc2ncnc(-c3cc(Cl)cc4c3OC(C(=O)N3CCNCC3)C4)c2s1]

In embodiments, the compound has the formula:

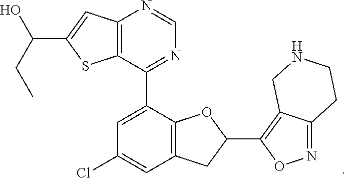 [SMILES: CCC(O)c1cc2ncnc(-c3cc(Cl)cc4c3OC(c3onc5c3CNCC5)C4)c2s1]

In embodiments, the compound has the formula:

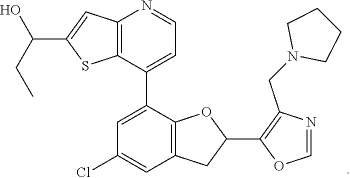 [SMILES: CCC(O)c1cc2nccc(-c3cc(Cl)cc4c3OC(c3ocnc3CN3CCCC3)C4)c2s1]

In embodiments, the compound has the formula:

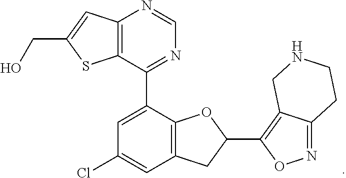 [SMILES: OCc1cc2ncnc(-c3cc(Cl)cc4c3OC(c3onc5c3CNCC5)C4)c2s1]

In embodiments, the compound has the formula:

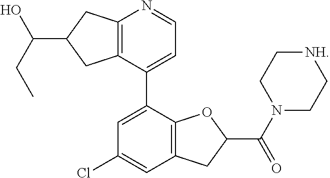 [SMILES: CCC(O)C1Cc2nccc(-c3cc(Cl)cc4c3OC(C(=O)N3CCNCC3)C4)c2C1]

In embodiments, the compound has the formula:

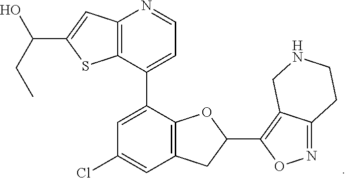 [SMILES: CCC(O)c1cc2nccc(-c3cc(Cl)cc4c3OC(c3onc5c3CNCC5)C4)c2s1]

In embodiments, the compound has the formula:

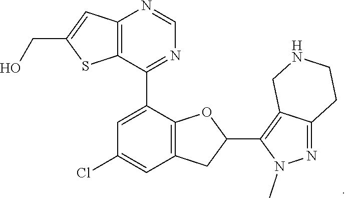 [SMILES: Cn1nc2c(c1C1Cc3cc(Cl)cc(-c4ncnc5cc(CO)sc45)c3O1)CNCC2]

In embodiments, the compound has the formula:

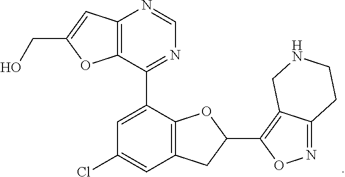 [SMILES: OCc1cc2ncnc(-c3cc(Cl)cc4c3OC(c3onc5c3CNCC5)C4)c2o1]

In embodiments, the compound has the formula:

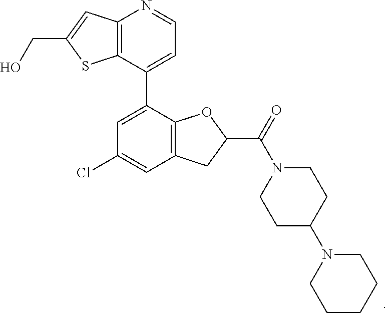 [SMILES: O=C(C1Cc2cc(Cl)cc(-c3ccnc4cc(CO)sc34)c2O1)N1CCC(N2CCCCC2)CC1]

In embodiments, the compound has the formula:

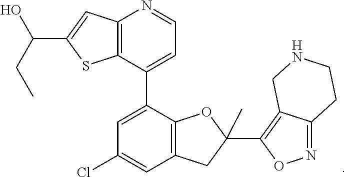 [SMILES: CCC(O)c1cc2nccc(-c3cc(Cl)cc4c3OC(C)(c3onc5c3CNCC5)C4)c2s1]

In embodiments, the compound has the formula:

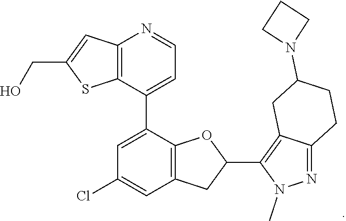 [SMILES: Cn1nc2c(c1C1Cc3cc(Cl)cc(-c4ccnc5cc(CO)sc45)c3O1)CC(N1CCC1)CC2]

In embodiments, the compound has the formula:

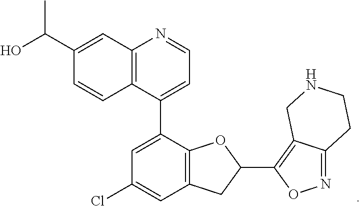 [SMILES: CC(O)c1ccc2c(-c3cc(Cl)cc4c3OC(c3onc5c3CNCC5)C4)ccnc2c1]

In embodiments, the compound has the formula:

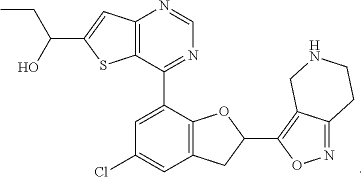 [SMILES: CCC(O)c1cc2ncnc(-c3cc(Cl)cc4c3OC(c3onc5c3CNCC5)C4)c2s1]

In embodiments, the compound has the formula:

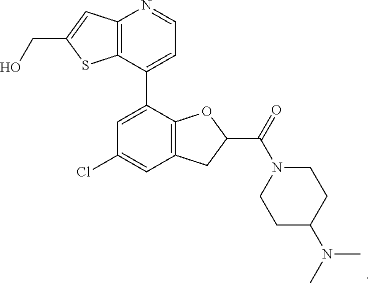 [SMILES: CN(C)C1CCN(C(=O)C2Cc3cc(Cl)cc(-c4ccnc5cc(CO)sc45)c3O2)CC1]

In embodiments, the compound has the formula:

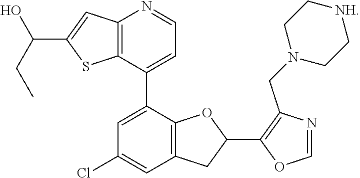 [SMILES: CCC(O)c1cc2nccc(-c3cc(Cl)cc4c3OC(c3ocnc3CN3CCNCC3)C4)c2s1]

In embodiments, the compound has the formula:

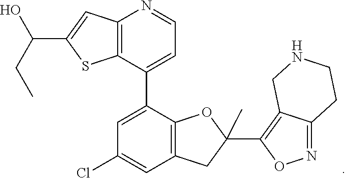 [SMILES: CCC(O)c1cc2nccc(-c3cc(Cl)cc4c3OC(C)(c3onc5c3CNCC5)C4)c2s1]

In embodiments, the compound has the formula:

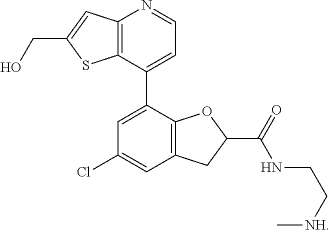 [SMILES: CNCCNC(=O)C1Cc2cc(Cl)cc(-c3ccnc4cc(CO)sc34)c2O1]

In embodiments, the compound has the formula:

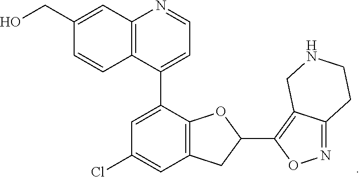 [SMILES: OCc1ccc2c(-c3cc(Cl)cc4c3OC(c3onc5c3CNCC5)C4)ccnc2c1]

In embodiments, the compound has the formula:

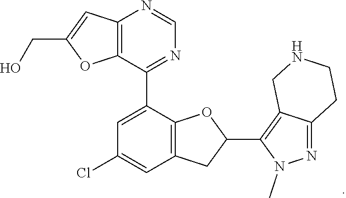 [SMILES: Cn1nc2c(c1C1Cc3cc(Cl)cc(-c4ncnc5cc(CO)oc45)c3O1)CNCC2]

In embodiments, the compound has the formula:

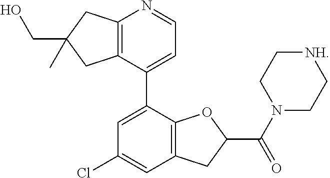 [SMILES: CC1(CO)Cc2nccc(-c3cc(Cl)cc4c3OC(C(=O)N3CCNCC3)C4)c2C1]

In embodiments, the compound has the formula:

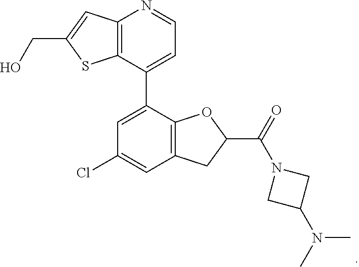 [SMILES: CN(C)C1CN(C(=O)C2Cc3cc(Cl)cc(-c4ccnc5cc(CO)sc45)c3O2)C1]

In embodiments, the compound has the formula:

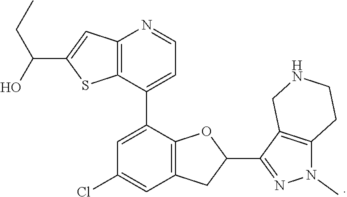 [SMILES: CCC(O)c1cc2nccc(-c3cc(Cl)cc4c3OC(c3nn(C)c5c3CNCC5)C4)c2s1]

In embodiments, the compound has the formula:

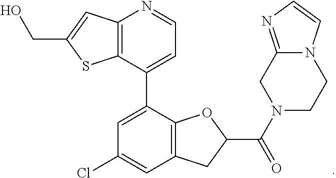 [SMILES: O=C(C1Cc2cc(Cl)cc(-c3ccnc4cc(CO)sc34)c2O1)N1CCn2ccnc2C1]

In embodiments, the compound has the formula:

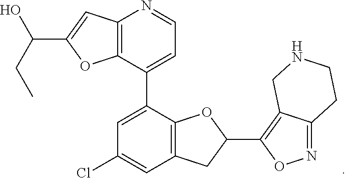 [SMILES: CCC(O)c1cc2nccc(-c3cc(Cl)cc4c3OC(c3onc5c3CNCC5)C4)c2o1]

In embodiments, the compound has the formula:

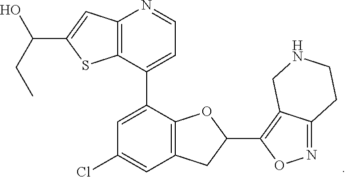 [SMILES: CCC(O)c1cc2nccc(-c3cc(Cl)cc4c3OC(c3onc5c3CNCC5)C4)c2s1]

In embodiments, the compound has the formula:

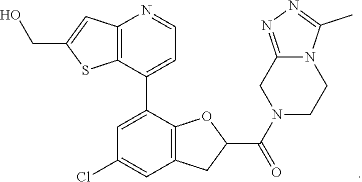 [SMILES: Cc1nnc2n1CCN(C(=O)C1Cc3cc(Cl)cc(-c4ccnc5cc(CO)sc45)c3O1)C2]

In embodiments, the compound has the formula:

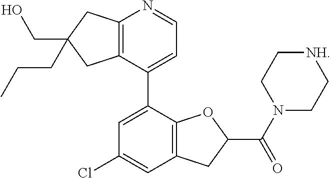 [SMILES: CCCC1(CO)Cc2nccc(-c3cc(Cl)cc4c3OC(C(=O)N3CCNCC3)C4)c2C1]

In embodiments, the compound has the formula:

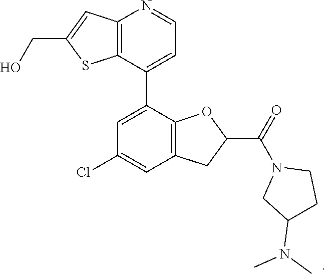 [SMILES: CN(C)C1CCN(C(=O)C2Cc3cc(Cl)cc(-c4ccnc5cc(CO)sc45)c3O2)C1]

In embodiments, the compound has the formula:

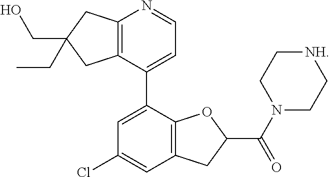 [SMILES: CCC1(CO)Cc2nccc(-c3cc(Cl)cc4c3OC(C(=O)N3CCNCC3)C4)c2C1]

In embodiments, the compound has the formula:

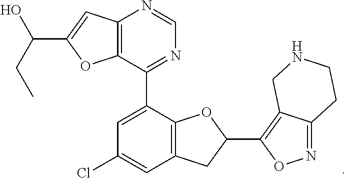 [SMILES: CCC(O)c1cc2ncnc(-c3cc(Cl)cc4c3OC(c3onc5c3CNCC5)C4)c2o1]

In embodiments, the compound has the formula:

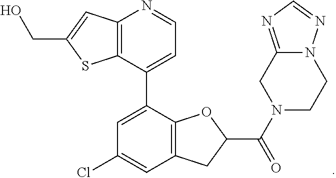 [SMILES: O=C(C1Cc2cc(Cl)cc(-c3ccnc4cc(CO)sc34)c2O1)N1CCn2ncnc2C1]

In embodiments, the compound has the formula:

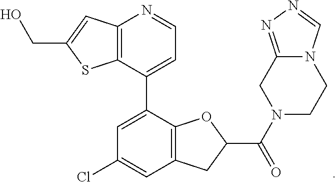 [SMILES: O=C(C1Cc2cc(Cl)cc(-c3ccnc4cc(CO)sc34)c2O1)N1CCn2cnnc2C1]

In embodiments, the compound has the formula:

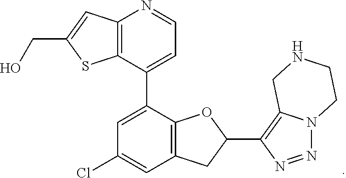 [SMILES: OCc1cc2nccc(-c3cc(Cl)cc4c3OC(c3nnn5c3CNCC5)C4)c2s1]

In embodiments, the compound has the formula:

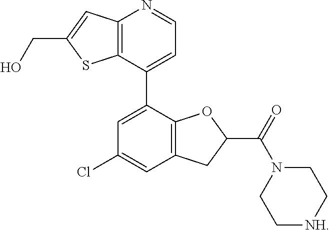 [SMILES: O=C(C1Cc2cc(Cl)cc(-c3ccnc4cc(CO)sc34)c2O1)N1CCNCC1]

In embodiments, the compound has the formula:

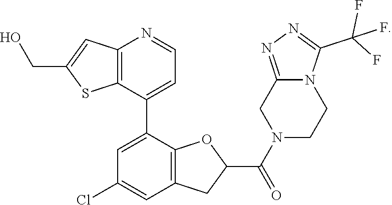 [SMILES: O=C(C1Cc2cc(Cl)cc(-c3ccnc4cc(CO)sc34)c2O1)N1CCn2c(nnc2C(F)(F)F)C1]

In embodiments, the compound has the formula:

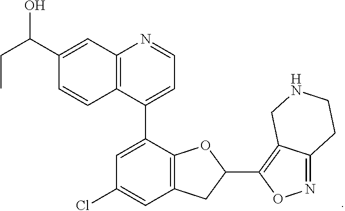 [SMILES: CCC(O)c1ccc2c(-c3cc(Cl)cc4c3OC(c3onc5c3CNCC5)C4)ccnc2c1]

In embodiments, the compound has the formula:

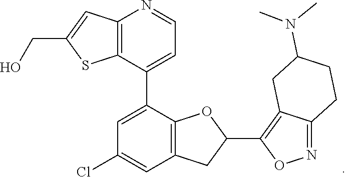 [SMILES: CN(C)C1CCc2noc(C3Cc4cc(Cl)cc(-c5ccnc6cc(CO)sc56)c4O3)c2C1]

In embodiments, the compound has the formula:

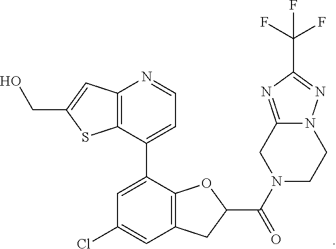 [SMILES: O=C(C1Cc2cc(Cl)cc(-c3ccnc4cc(CO)sc34)c2O1)N1CCn2nc(C(F)(F)F)nc2C1]

In embodiments, the compound has the formula:

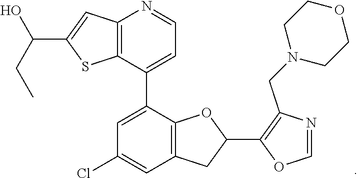 [SMILES: CCC(O)c1cc2nccc(-c3cc(Cl)cc4c3OC(c3ocnc3CN3CCOCC3)C4)c2s1]

In embodiments, the compound has the formula:

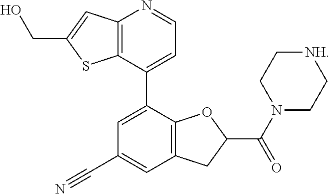 [SMILES: N#Cc1cc2c(c(-c3ccnc4cc(CO)sc34)c1)OC(C(=O)N1CCNCC1)C2]

In embodiments, the compound has the formula:

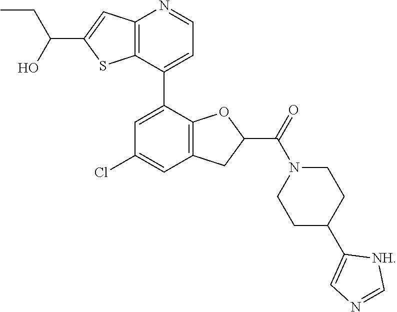 [SMILES: CCC(O)c1cc2nccc(-c3cc(Cl)cc4c3OC(C(=O)N3CCC(c5cnc[nH]5)CC3)C4)c2s1]

In embodiments, the compound has the formula:

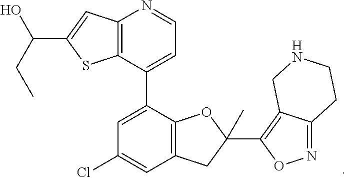 [SMILES: CCC(O)c1cc2nccc(-c3cc(Cl)cc4c3OC(C)(c3onc5c3CNCC5)C4)c2s1]

In embodiments, the compound has the formula:

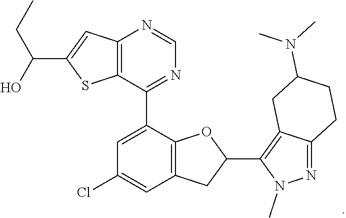 [SMILES: CCC(O)c1cc2ncnc(-c3cc(Cl)cc4c3OC(c3c5c(nn3C)CCC(N(C)C)C5)C4)c2s1]

In embodiments, the compound has the formula:

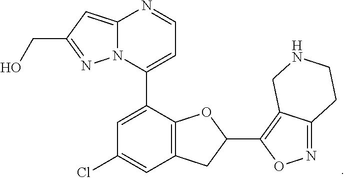 [SMILES: OCc1cc2nccc(-c3cc(Cl)cc4c3OC(c3onc5c3CNCC5)C4)n2n1]

In embodiments, the compound has the formula:

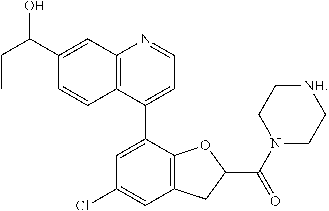 [SMILES: CCC(O)c1ccc2c(-c3cc(Cl)cc4c3OC(C(=O)N3CCNCC3)C4)ccnc2c1]

In embodiments, the compound has the formula:

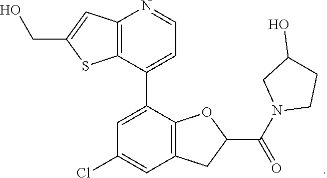 [SMILES: O=C(C1Cc2cc(Cl)cc(-c3ccnc4cc(CO)sc34)c2O1)N1CCC(O)C1]

In embodiments, the compound has the formula:

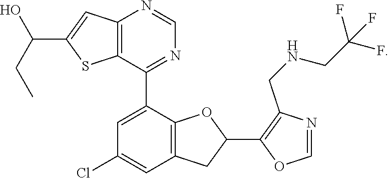 [SMILES: CCC(O)c1cc2ncnc(-c3cc(Cl)cc4c3OC(c3ocnc3CNCC(F)(F)F)C4)c2s1]

In embodiments, the compound has the formula:

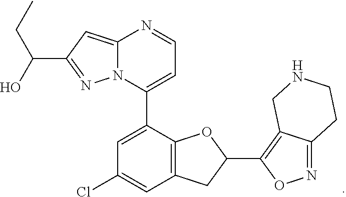 [SMILES: CCC(O)c1cc2nccc(-c3cc(Cl)cc4c3OC(c3onc5c3CNCC5)C4)n2n1]

In embodiments, the compound has the formula:

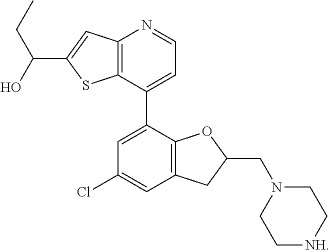 [SMILES: CCC(O)c1cc2nccc(-c3cc(Cl)cc4c3OC(CN3CCNCC3)C4)c2s1]

In embodiments, the compound has the formula:

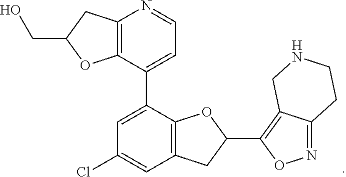 [SMILES: OCC1Cc2nccc(-c3cc(Cl)cc4c3OC(c3onc5c3CNCC5)C4)c2O1]

In embodiments, the compound has the formula:

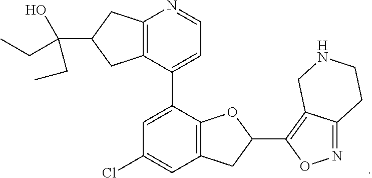 [SMILES: CCC(O)(CC)C1Cc2nccc(-c3cc(Cl)cc4c3OC(c3onc5c3CNCC5)C4)c2C1]

In embodiments, the compound has the formula:

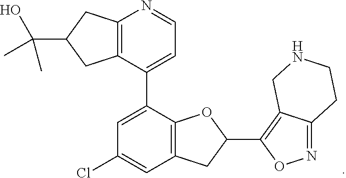 [SMILES: CC(C)(O)C1Cc2nccc(-c3cc(Cl)cc4c3OC(c3onc5c3CNCC5)C4)c2C1]

In embodiments, the compound has the formula:

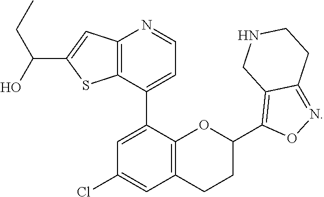 [SMILES: CCC(O)c1cc2nccc(-c3cc(Cl)cc4c3OC(c3onc5c3CNCC5)CC4)c2s1]

In embodiments, the compound has the formula:

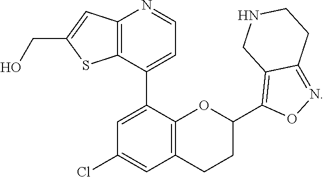 [SMILES: OCc1cc2nccc(-c3cc(Cl)cc4c3OC(c3onc5c3CNCC5)CC4)c2s1]

In embodiments, the compound has the formula:

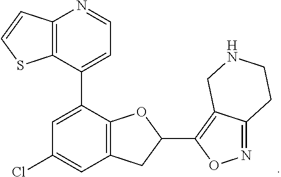 [SMILES: Clc1cc2c(c(-c3ccnc4ccsc34)c1)OC(c1onc3c1CNCC3)C2]

In embodiments, the compound has the formula:

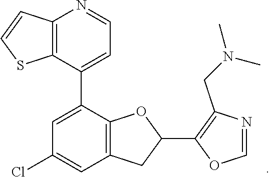 [SMILES: CN(C)Cc1ncoc1C1Cc2cc(Cl)cc(-c3ccnc4ccsc34)c2O1]

In embodiments, the compound has the formula:

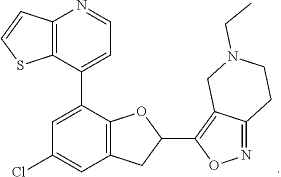 [SMILES: CCN1CCc2noc(C3Cc4cc(Cl)cc(-c5ccnc6ccsc56)c4O3)c2C1]

In embodiments, the compound has the formula:

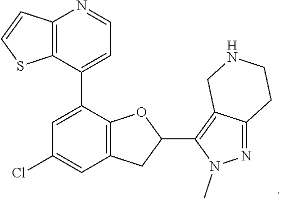 [SMILES: Cn1nc2c(c1C1Cc3cc(Cl)cc(-c4ccnc5ccsc45)c3O1)CNCC2]

In embodiments, the compound has the formula:

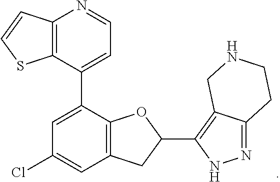 [SMILES: Clc1cc2c(c(-c3ccnc4ccsc34)c1)OC(c1[nH]nc3c1CNCC3)C2]

In embodiments, the compound has the formula:

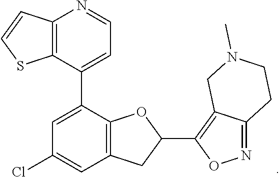 [SMILES: CN1CCc2noc(C3Cc4cc(Cl)cc(-c5ccnc6ccsc56)c4O3)c2C1]

In embodiments, the compound has the formula:

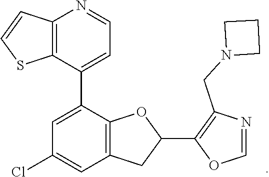 [SMILES: Clc1cc2c(c(-c3ccnc4ccsc34)c1)OC(c1ocnc1CN1CCC1)C2]

In embodiments, the compound has the formula:

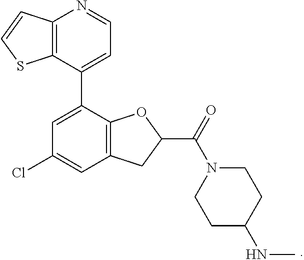 [SMILES: CNC1CCN(C(=O)C2Cc3cc(Cl)cc(-c4ccnc5ccsc45)c3O2)CC1]

In embodiments, the compound has the formula:

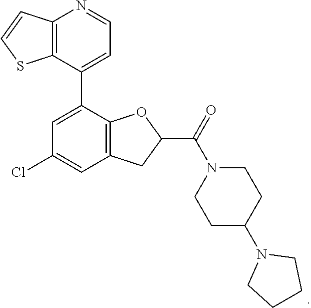 [SMILES: O=C(C1Cc2cc(Cl)cc(-c3ccnc4ccsc34)c2O1)N1CCC(N2CCCC2)CC1]

In embodiments, the compound has the formula:

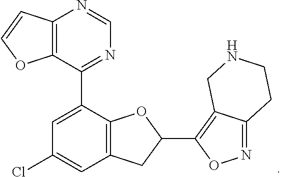 [SMILES: Clc1cc2c(c(-c3ncnc4ccoc34)c1)OC(c1onc3c1CNCC3)C2]

In embodiments, the compound has the formula:

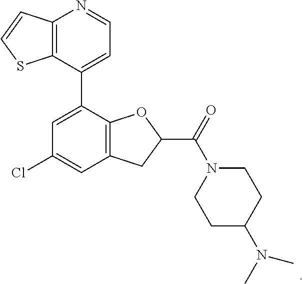 [SMILES: CN(C)C1CCN(C(=O)C2Cc3cc(Cl)cc(-c4ccnc5ccsc45)c3O2)CC1]

In embodiments, the compound has the formula:

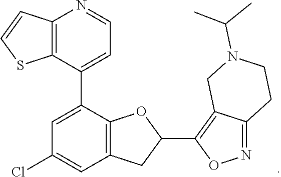 [SMILES: CC(C)N1CCc2noc(C3Cc4cc(Cl)cc(-c5ccnc6ccsc56)c4O3)c2C1]

In embodiments, the compound has the formula:

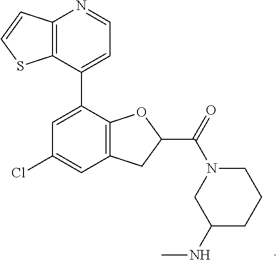 [SMILES: CNC1CCCN(C(=O)C2Cc3cc(Cl)cc(-c4ccnc5ccsc45)c3O2)C1]

In embodiments, the compound has the formula:

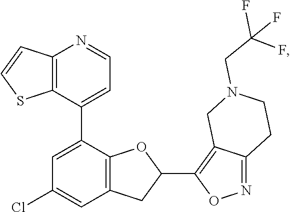 [SMILES: FC(F)(F)CN1CCc2noc(C3Cc4cc(Cl)cc(-c5ccnc6ccsc56)c4O3)c2C1]

In embodiments, the compound has the formula:

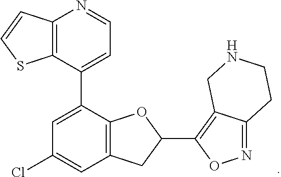 [SMILES: Clc1cc2c(c(-c3ccnc4ccsc34)c1)OC(c1onc3c1CNCC3)C2]

In embodiments, the compound has the formula:

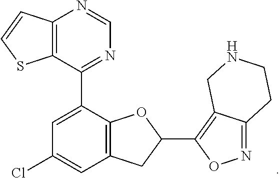 [SMILES: Clc1cc2c(c(-c3ncnc4ccsc34)c1)OC(c1onc3c1CNCC3)C2]

In embodiments, the compound has the formula:

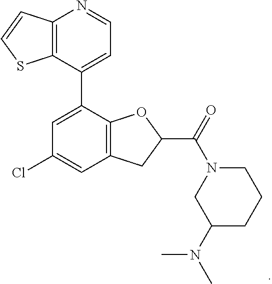 [SMILES: CN(C)C1CCCN(C(=O)C2Cc3cc(Cl)cc(-c4ccnc5ccsc45)c3O2)C1]

In embodiments, the compound has the formula:

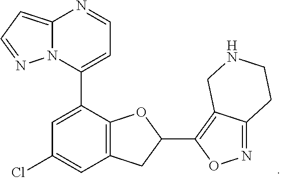 [SMILES: Clc1cc2c(c(-c3ccnc4ccnn34)c1)OC(c1onc3c1CNCC3)C2]

In embodiments, the compound has the formula:

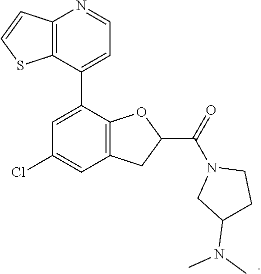 [SMILES: CN(C)C1CCN(C(=O)C2Cc3cc(Cl)cc(-c4ccnc5ccsc45)c3O2)C1]

In embodiments, the compound has the formula:

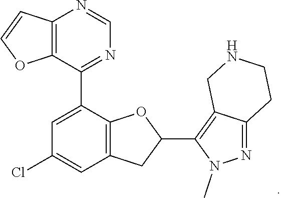 [SMILES: Cn1nc2c(c1C1Cc3cc(Cl)cc(-c4ncnc5ccoc45)c3O1)CNCC2]

In embodiments, the compound has the formula:

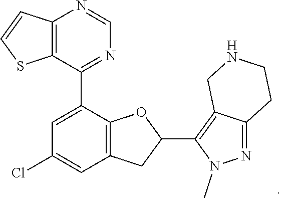 [SMILES: Cn1nc2c(c1C1Cc3cc(Cl)cc(-c4ncnc5ccsc45)c3O1)CNCC2]

In embodiments, the compound has the formula:

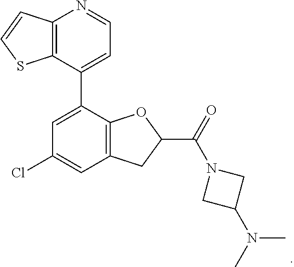 [SMILES: CN(C)C1CN(C(=O)C2Cc3cc(Cl)cc(-c4ccnc5ccsc45)c3O2)C1]

In embodiments, the compound has the formula:

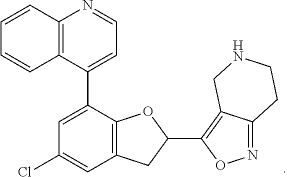 [SMILES: Clc1cc2c(c(-c3ccnc4ccccc34)c1)OC(c1onc3c1CNCC3)C2]

In embodiments, the compound has the formula:

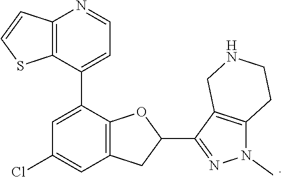 [SMILES: Cn1nc(C2Cc3cc(Cl)cc(-c4ccnc5ccsc45)c3O2)c2c1CCNC2]

In embodiments, the compound has the formula:

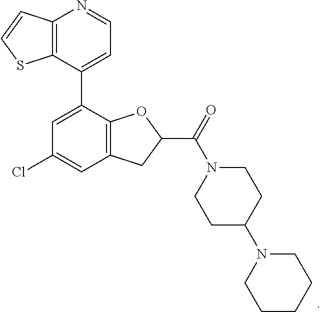 [SMILES: O=C(C1Cc2cc(Cl)cc(-c3ccnc4ccsc34)c2O1)N1CCC(N2CCCCC2)CC1]

In embodiments, the compound has the formula:

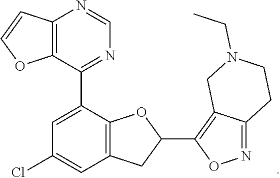 [SMILES: CCN1CCc2noc(C3Cc4cc(Cl)cc(-c5ncnc6ccoc56)c4O3)c2C1]

In embodiments, the compound has the formula:

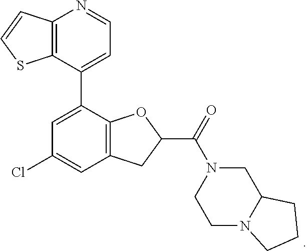 [SMILES: O=C(C1Cc2cc(Cl)cc(-c3ccnc4ccsc34)c2O1)N1CCN2CCCC2C1]

In embodiments, the compound has the formula:

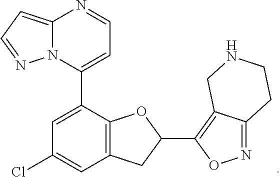 [SMILES: Clc1cc2c(c(-c3ccnc4ccnn34)c1)OC(c1onc3c1CNCC3)C2]

In embodiments, the compound has the formula:

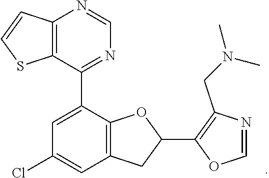 [SMILES: CN(C)Cc1ncoc1C1Cc2cc(Cl)cc(-c3ncnc4ccsc34)c2O1]

In embodiments, the compound has the formula:

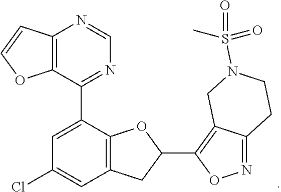 [SMILES: CS(=O)(=O)N1CCc2noc(C3Cc4cc(Cl)cc(-c5ncnc6ccoc56)c4O3)c2C1]

In embodiments, the compound has the formula:

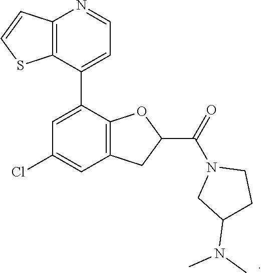 [SMILES: CN(C)C1CCN(C(=O)C2Cc3cc(Cl)cc(-c4ccnc5ccsc45)c3O2)C1]

In embodiments, the compound has the formula:

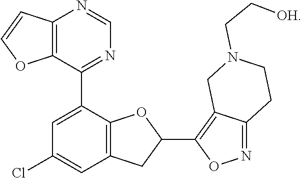 [SMILES: OCCN1CCc2noc(C3Cc4cc(Cl)cc(-c5ncnc6ccoc56)c4O3)c2C1]

In embodiments, the compound has the formula:

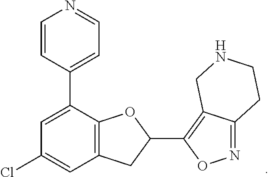 [SMILES: Clc1cc2c(c(-c3ccncc3)c1)OC(c1onc3c1CNCC3)C2]

In embodiments, the compound has the formula:

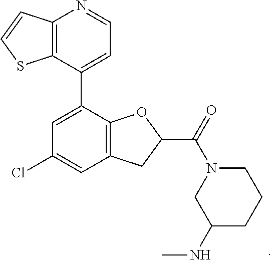 [SMILES: CNC1CCCN(C(=O)C2Cc3cc(Cl)cc(-c4ccnc5ccsc45)c3O2)C1]

In embodiments, the compound has the formula:

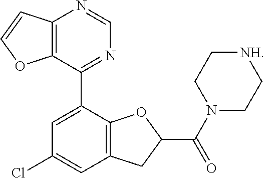 [SMILES: O=C(C1Cc2cc(Cl)cc(-c3ncnc4ccoc34)c2O1)N1CCNCC1]

In embodiments, the compound has the formula:

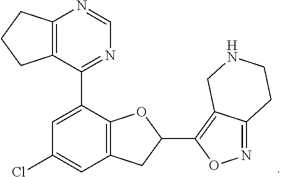 [SMILES: Clc1cc2c(c(-c3ncnc4c3CCC4)c1)OC(c1onc3c1CNCC3)C2]

In embodiments, the compound has the formula:

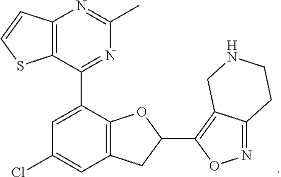 [SMILES: Cc1nc(-c2cc(Cl)cc3c2OC(c2onc4c2CNCC4)C3)c2sccc2n1]

In embodiments, the compound has the formula:

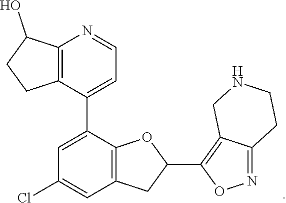 [SMILES: OC1CCc2c(-c3cc(Cl)cc4c3OC(c3onc5c3CNCC5)C4)ccnc21]

In embodiments, the compound has the formula:

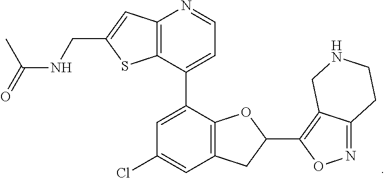 [SMILES: CC(=O)NCc1cc2nccc(-c3cc(Cl)cc4c3OC(c3onc5c3CNCC5)C4)c2s1]

In embodiments, the compound has the formula:

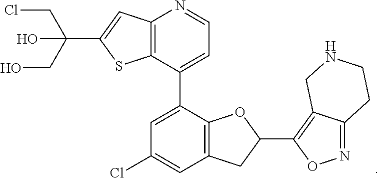 [SMILES: OCC(O)(CCl)c1cc2nccc(-c3cc(Cl)cc4c3OC(c3onc5c3CNCC5)C4)c2s1]

In embodiments, the compound has the formula:

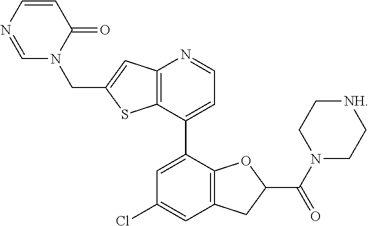 [SMILES: O=C(C1Cc2cc(Cl)cc(-c3ccnc4cc(Cn5cnccc5=O)sc34)c2O1)N1CCNCC1]

In embodiments, the compound has the formula:

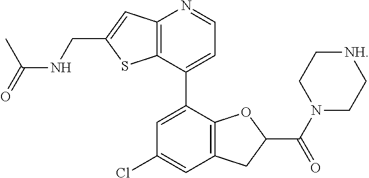 [SMILES: CC(=O)NCc1cc2nccc(-c3cc(Cl)cc4c3OC(C(=O)N3CCNCC3)C4)c2s1]

In embodiments, the compound has the formula:

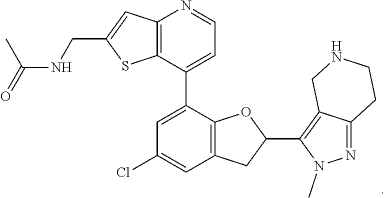 [SMILES: CC(=O)NCc1cc2nccc(-c3cc(Cl)cc4c3OC(c3c5c(nn3C)CCNC5)C4)c2s1]

In embodiments, the compound has the formula:

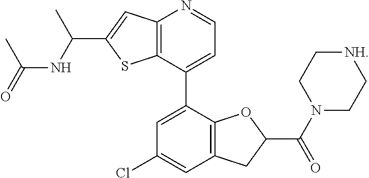 [SMILES: CC(=O)NC(C)c1cc2nccc(-c3cc(Cl)cc4c3OC(C(=O)N3CCNCC3)C4)c2s1]

In embodiments, the compound has the formula:

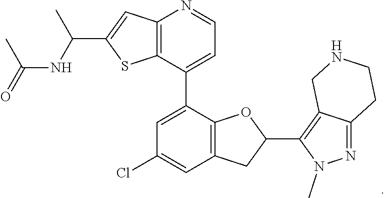 [SMILES: CC(=O)NC(C)c1cc2nccc(-c3cc(Cl)cc4c3OC(c3c5c(nn3C)CCNC5)C4)c2s1]

In embodiments, the compound has the formula:

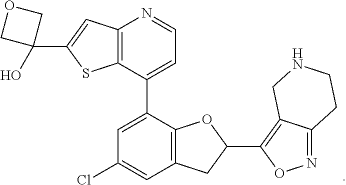 [SMILES: OC1(c2cc3nccc(-c4cc(Cl)cc5c4OC(c4onc6c4CNCC6)C5)c3s2)COC1]

In embodiments, the compound has the formula:

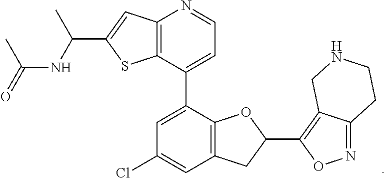 [SMILES: CC(=O)NC(C)c1cc2nccc(-c3cc(Cl)cc4c3OC(c3onc5c3CNCC5)C4)c2s1]

In embodiments, the compound has the formula:

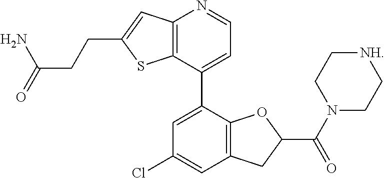 [SMILES: NC(=O)CCc1cc2nccc(-c3cc(Cl)cc4c3OC(C(=O)N3CCNCC3)C4)c2s1]

In embodiments, the compound has the formula:

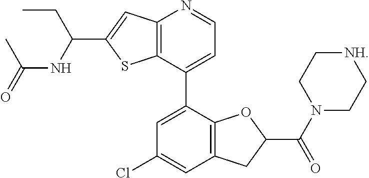 [SMILES: CCC(NC(C)=O)c1cc2nccc(-c3cc(Cl)cc4c3OC(C(=O)N3CCNCC3)C4)c2s1]

In embodiments, the compound has the formula:

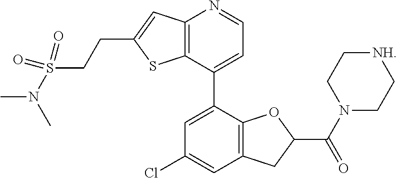 [SMILES: CN(C)S(=O)(=O)CCc1cc2nccc(-c3cc(Cl)cc4c3OC(C(=O)N3CCNCC3)C4)c2s1]

In embodiments, the compound has the formula:

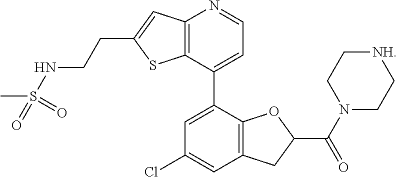 [SMILES: CS(=O)(=O)NCCc1cc2nccc(-c3cc(Cl)cc4c3OC(C(=O)N3CCNCC3)C4)c2s1]

In embodiments, the compound has the formula:

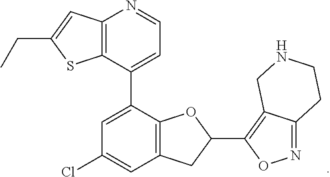 [SMILES: CCc1cc2nccc(-c3cc(Cl)cc4c3OC(c3onc5c3CNCC5)C4)c2s1]

In embodiments, the compound has the formula:

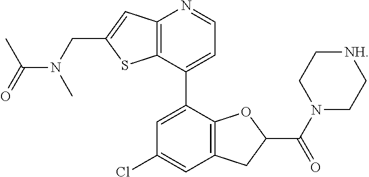 [SMILES: CC(=O)N(C)Cc1cc2nccc(-c3cc(Cl)cc4c3OC(C(=O)N3CCNCC3)C4)c2s1]

In embodiments, the compound has the formula:

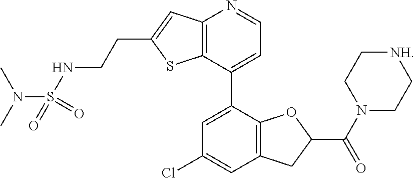 [SMILES: CN(C)S(=O)(=O)NCCc1cc2nccc(-c3cc(Cl)cc4c3OC(C(=O)N3CCNCC3)C4)c2s1]

In embodiments, the compound has the formula:

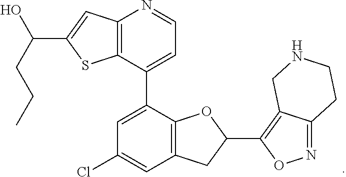 [SMILES: CCCC(O)c1cc2nccc(-c3cc(Cl)cc4c3OC(c3onc5c3CNCC5)C4)c2s1]

In embodiments, the compound has the formula:

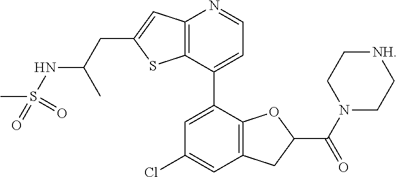 [SMILES: CC(Cc1cc2nccc(-c3cc(Cl)cc4c3OC(C(=O)N3CCNCC3)C4)c2s1)NS(C)(=O)=O]

In embodiments, the compound has the formula:

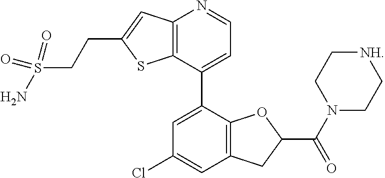 [SMILES: NS(=O)(=O)CCc1cc2nccc(-c3cc(Cl)cc4c3OC(C(=O)N3CCNCC3)C4)c2s1]

In embodiments, the compound has the formula:

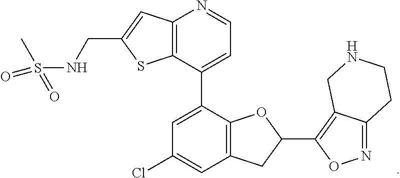 [SMILES: CS(=O)(=O)NCc1cc2nccc(-c3cc(Cl)cc4c3OC(c3onc5c3CNCC5)C4)c2s1]

In embodiments, the compound has the formula:

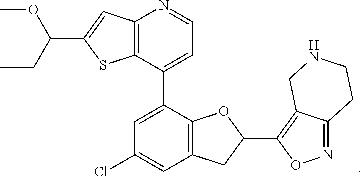 [SMILES: CCC(OC)c1cc2nccc(-c3cc(Cl)cc4c3OC(c3onc5c3CNCC5)C4)c2s1]

In embodiments, the compound has the formula:

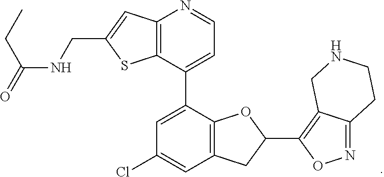 [SMILES: CCC(=O)NCc1cc2nccc(-c3cc(Cl)cc4c3OC(c3onc5c3CNCC5)C4)c2s1]

In embodiments, the compound has the formula:

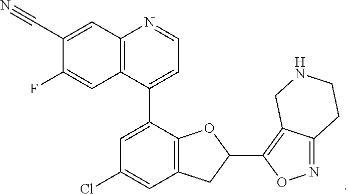 [SMILES: N#Cc1cc2nccc(-c3cc(Cl)cc4c3OC(c3onc5c3CNCC5)C4)c2cc1F]

In embodiments, the compound has the formula:

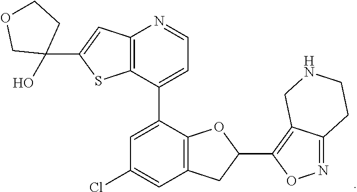 [SMILES: OC1(c2cc3nccc(-c4cc(Cl)cc5c4OC(c4onc6c4CNCC6)C5)c3s2)CCOC1]

In embodiments, the compound has the formula:

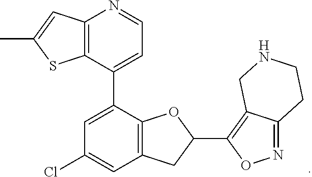 [SMILES: Cc1cc2nccc(-c3cc(Cl)cc4c3OC(c3onc5c3CNCC5)C4)c2s1]

In embodiments, the compound has the formula:

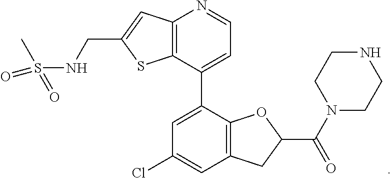 [SMILES: CS(=O)(=O)NCc1cc2nccc(-c3cc(Cl)cc4c3OC(C(=O)N3CCNCC3)C4)c2s1]

In embodiments, the compound has the formula:

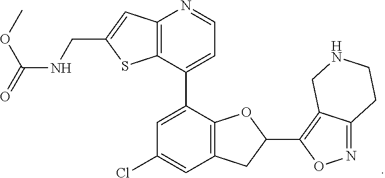 [SMILES: COC(=O)NCc1cc2nccc(-c3cc(Cl)cc4c3OC(c3onc5c3CNCC5)C4)c2s1]

In embodiments, the compound has the formula:

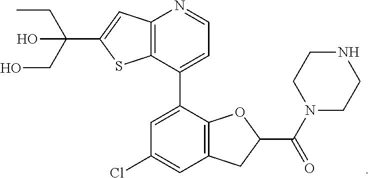 [SMILES: CCC(O)(CO)c1cc2nccc(-c3cc(Cl)cc4c3OC(C(=O)N3CCNCC3)C4)c2s1]

In embodiments, the compound has the formula:

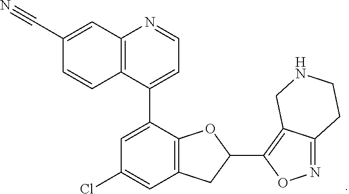 [SMILES: N#Cc1ccc2c(-c3cc(Cl)cc4c3OC(c3onc5c3CNCC5)C4)ccnc2c1]

In embodiments, the compound has the formula:

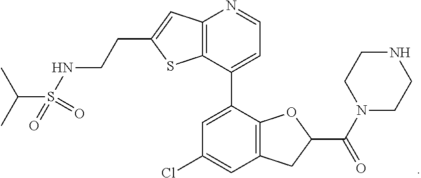 [SMILES: CC(C)S(=O)(=O)NCCc1cc2nccc(-c3cc(Cl)cc4c3OC(C(=O)N3CCNCC3)C4)c2s1]

In embodiments, the compound has the formula:

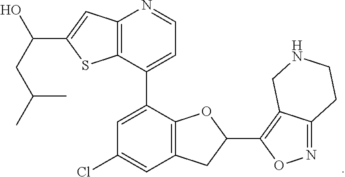 [SMILES: CC(C)CC(O)c1cc2nccc(-c3cc(Cl)cc4c3OC(c3onc5c3CNCC5)C4)c2s1]

In embodiments, the compound has the formula:

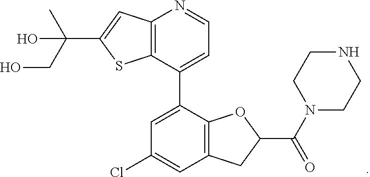 [SMILES: CC(O)(CO)c1cc2nccc(-c3cc(Cl)cc4c3OC(C(=O)N3CCNCC3)C4)c2s1]

In embodiments, the compound has the formula:

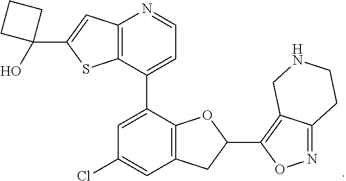 [SMILES: OC1(c2cc3nccc(-c4cc(Cl)cc5c4OC(c4onc6c4CNCC6)C5)c3s2)CCC1]

In embodiments, the compound has the formula:

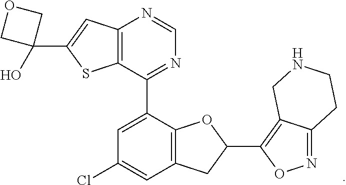 [SMILES: OC1(c2cc3ncnc(-c4cc(Cl)cc5c4OC(c4onc6c4CNCC6)C5)c3s2)COC1]

In embodiments, the compound has the formula:

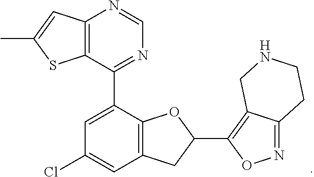 [SMILES: Cc1cc2ncnc(-c3cc(Cl)cc4c3OC(c3onc5c3CNCC5)C4)c2s1]

In embodiments, the compound has the formula:

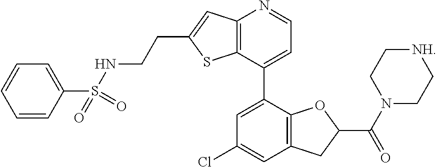 [SMILES: O=C(C1Cc2cc(Cl)cc(-c3ccnc4cc(CCNS(=O)(=O)c5ccccc5)sc34)c2O1)N1CCNCC1]

In embodiments, the compound has the formula:

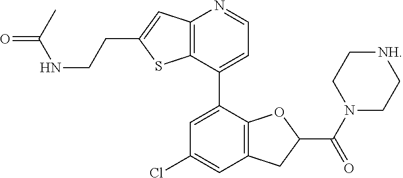 [SMILES: CC(=O)NCCc1cc2nccc(-c3cc(Cl)cc4c3OC(C(=O)N3CCNCC3)C4)c2s1]

In embodiments, the compound has the formula:

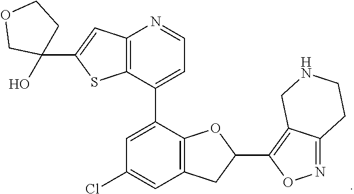 [SMILES: OC1(c2cc3nccc(-c4cc(Cl)cc5c4OC(c4onc6c4CNCC6)C5)c3s2)CCOC1]

In embodiments, the compound has the formula:

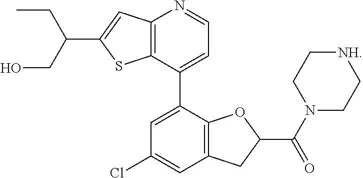 [SMILES: CCC(CO)c1cc2nccc(-c3cc(Cl)cc4c3OC(C(=O)N3CCNCC3)C4)c2s1]

In embodiments, the compound has the formula:

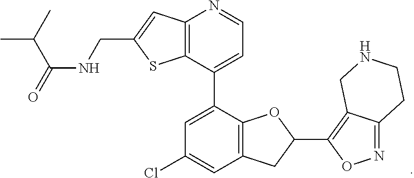 [SMILES: CC(C)C(=O)NCc1cc2nccc(-c3cc(Cl)cc4c3OC(c3onc5c3CNCC5)C4)c2s1]

In embodiments, the compound has the formula:

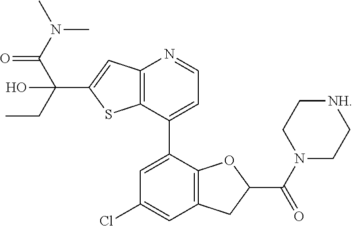 [SMILES: CCC(O)(C(=O)N(C)C)c1cc2nccc(-c3cc(Cl)cc4c3OC(C(=O)N3CCNCC3)C4)c2s1]

In embodiments, the compound has the formula:

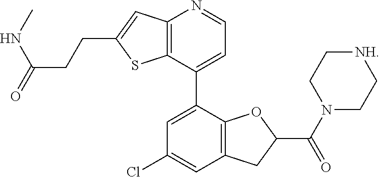 [SMILES: CNC(=O)CCc1cc2nccc(-c3cc(Cl)cc4c3OC(C(=O)N3CCNCC3)C4)c2s1]

In embodiments, the compound has the formula:

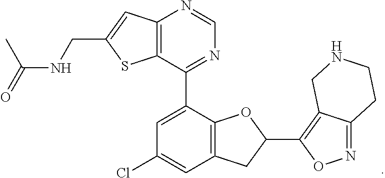 [SMILES: CC(=O)NCc1cc2ncnc(-c3cc(Cl)cc4c3OC(c3onc5c3CNCC5)C4)c2s1]

In embodiments, the compound has the formula:

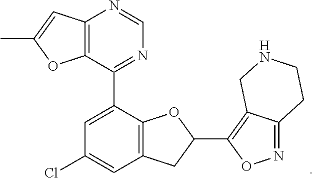 [SMILES: Cc1cc2ncnc(-c3cc(Cl)cc4c3OC(c3onc5c3CNCC5)C4)c2o1]

In embodiments, the compound has the formula:

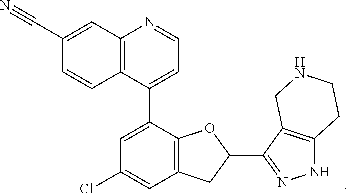 [SMILES: N#Cc1ccc2c(-c3cc(Cl)cc4c3OC(c3n[nH]c5c3CNCC5)C4)ccnc2c1]

In embodiments, the compound has the formula:

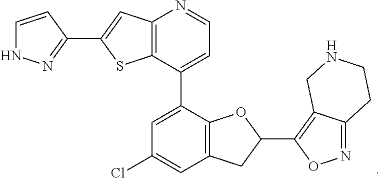 [SMILES: Clc1cc2c(c(-c3ccnc4cc(-c5cc[nH]n5)sc34)c1)OC(c1onc3c1CNCC3)C2]

In embodiments, the compound has the formula:

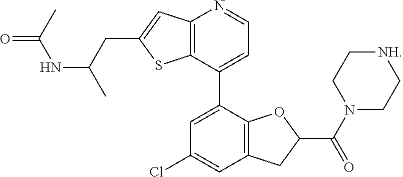 [SMILES: CC(=O)NC(C)Cc1cc2nccc(-c3cc(Cl)cc4c3OC(C(=O)N3CCNCC3)C4)c2s1]

In embodiments, the compound has the formula:

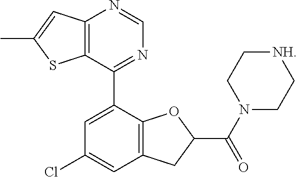 [SMILES: Cc1cc2ncnc(-c3cc(Cl)cc4c3OC(C(=O)N3CCNCC3)C4)c2s1]

In embodiments, the compound has the formula:

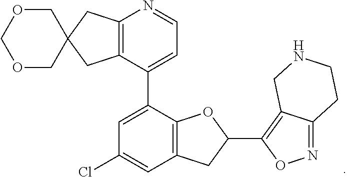 [SMILES: Clc1cc2c(c(-c3ccnc4c3CC3(COCOC3)C4)c1)OC(c1onc3c1CNCC3)C2]

In embodiments, the compound has the formula:

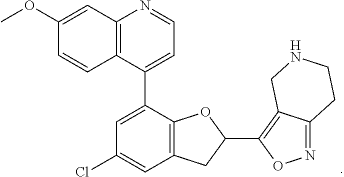 [SMILES: COc1ccc2c(-c3cc(Cl)cc4c3OC(c3onc5c3CNCC5)C4)ccnc2c1]

In embodiments, the compound has the formula:

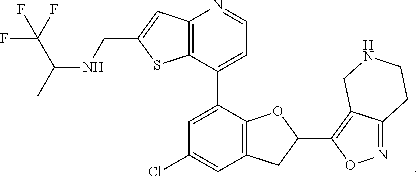 [SMILES: CC(NCc1cc2nccc(-c3cc(Cl)cc4c3OC(c3onc5c3CNCC5)C4)c2s1)C(F)(F)F]

In embodiments, the compound has the formula:

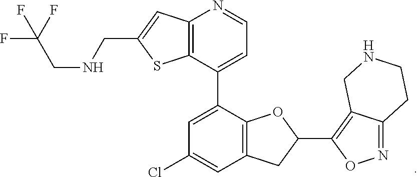 [SMILES: FC(F)(F)CNCc1cc2nccc(-c3cc(Cl)cc4c3OC(c3onc5c3CNCC5)C4)c2s1]

In embodiments, the compound has the formula:

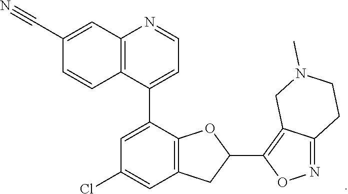 [SMILES: CN1CCc2noc(C3Cc4cc(Cl)cc(-c5ccnc6cc(C#N)ccc56)c4O3)c2C1]

In embodiments, the compound has the formula:

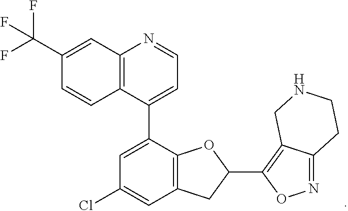 [SMILES: FC(F)(F)c1ccc2c(-c3cc(Cl)cc4c3OC(c3onc5c3CNCC5)C4)ccnc2c1]

In embodiments, the compound has the formula:

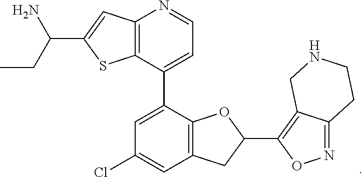 [SMILES: CCC(N)c1cc2nccc(-c3cc(Cl)cc4c3OC(c3onc5c3CNCC5)C4)c2s1]

In embodiments, the compound has the formula:

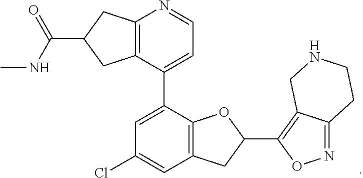 [SMILES: CNC(=O)C1Cc2nccc(-c3cc(Cl)cc4c3OC(c3onc5c3CNCC5)C4)c2C1]

In embodiments, the compound has the formula:

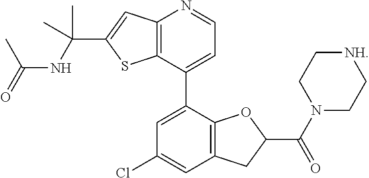 [SMILES: CC(=O)NC(C)(C)c1cc2nccc(-c3cc(Cl)cc4c3OC(C(=O)N3CCNCC3)C4)c2s1]

In embodiments, the compound has the formula:

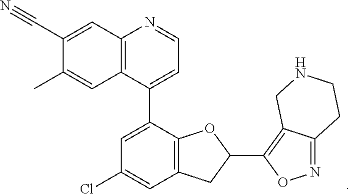 [SMILES: Cc1cc2c(-c3cc(Cl)cc4c3OC(c3onc5c3CNCC5)C4)ccnc2cc1C#N]

In embodiments, the compound has the formula:

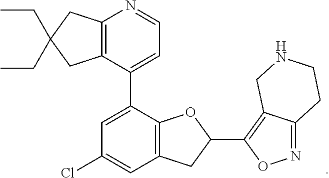 [SMILES: CCC1(CC)Cc2nccc(-c3cc(Cl)cc4c3OC(c3onc5c3CNCC5)C4)c2C1]

In embodiments, the compound has the formula:

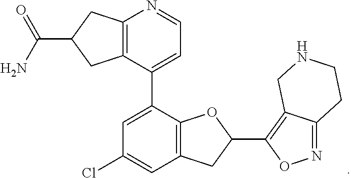 [SMILES: NC(=O)C1Cc2nccc(-c3cc(Cl)cc4c3OC(c3onc5c3CNCC5)C4)c2C1]

In embodiments, the compound has the formula:

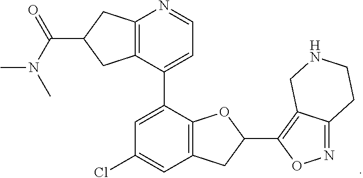 [SMILES: CN(C)C(=O)C1Cc2nccc(-c3cc(Cl)cc4c3OC(c3onc5c3CNCC5)C4)c2C1]

In embodiments, the compound has the formula:

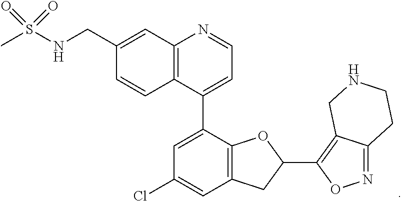 [SMILES: CS(=O)(=O)NCc1ccc2c(-c3cc(Cl)cc4c3OC(c3onc5c3CNCC5)C4)ccnc2c1]

In embodiments, the compound has the formula:

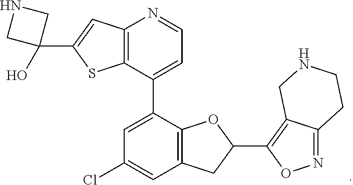 [SMILES: OC1(c2cc3nccc(-c4cc(Cl)cc5c4OC(c4onc6c4CNCC6)C5)c3s2)CNC1]

In embodiments, the compound has the formula:

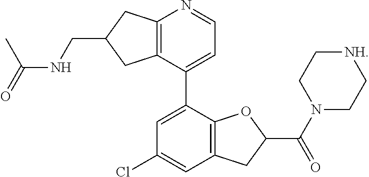 [SMILES: CC(=O)NCC1Cc2nccc(-c3cc(Cl)cc4c3OC(C(=O)N3CCNCC3)C4)c2C1]

In embodiments, the compound has the formula:

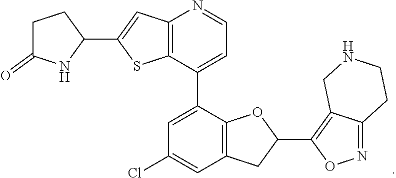 [SMILES: O=C1CCC(c2cc3nccc(-c4cc(Cl)cc5c4OC(c4onc6c4CNCC6)C5)c3s2)N1]

In embodiments, the compound has the formula:

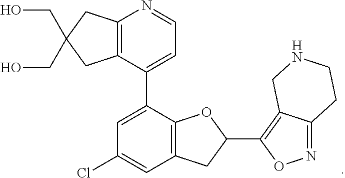 [SMILES: OCC1(CO)Cc2nccc(-c3cc(Cl)cc4c3OC(c3onc5c3CNCC5)C4)c2C1]

In embodiments, the compound has the formula:

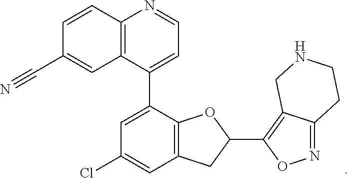 [SMILES: N#Cc1ccc2nccc(-c3cc(Cl)cc4c3OC(c3onc5c3CNCC5)C4)c2c1]

In embodiments, the compound has the formula:

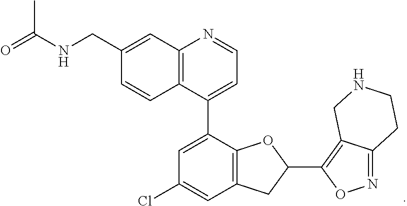 [SMILES: CC(=O)NCc1ccc2c(-c3cc(Cl)cc4c3OC(c3onc5c3CNCC5)C4)ccnc2c1]

In embodiments, the compound has the formula:

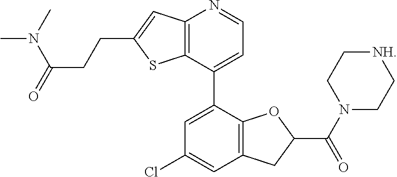 [SMILES: CN(C)C(=O)CCc1cc2nccc(-c3cc(Cl)cc4c3OC(C(=O)N3CCNCC3)C4)c2s1]

In embodiments, the compound has the formula:

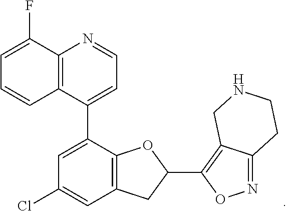 [SMILES: Fc1cccc2c(-c3cc(Cl)cc4c3OC(c3onc5c3CNCC5)C4)ccnc12]

In embodiments, the compound has the formula:

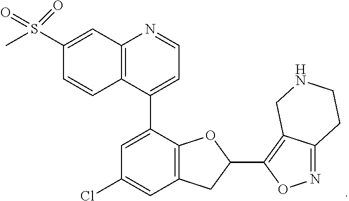 [SMILES: CS(=O)(=O)c1ccc2c(-c3cc(Cl)cc4c3OC(c3onc5c3CNCC5)C4)ccnc2c1]

In embodiments, the compound has the formula:

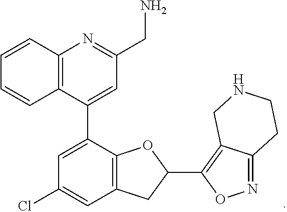 [SMILES: NCc1cc(-c2cc(Cl)cc3c2OC(c2onc4c2CNCC4)C3)c2ccccc2n1]

In embodiments, the compound has the formula:

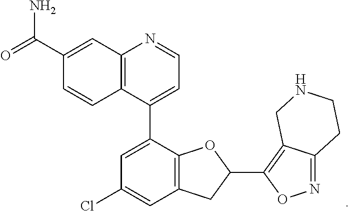 [SMILES: NC(=O)c1ccc2c(-c3cc(Cl)cc4c3OC(c3onc5c3CNCC5)C4)ccnc2c1]

In embodiments, the compound has the formula:

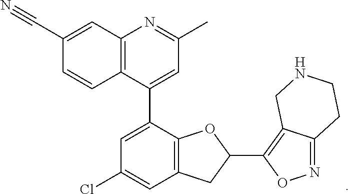 [SMILES: Cc1cc(-c2cc(Cl)cc3c2OC(c2onc4c2CNCC4)C3)c2ccc(C#N)cc2n1]

In embodiments, the compound has the formula:

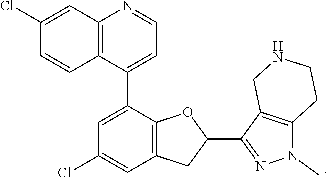 [SMILES: Cn1nc(C2Cc3cc(Cl)cc(-c4ccnc5cc(Cl)ccc45)c3O2)c2c1CCNC2]

In embodiments, the compound has the formula:

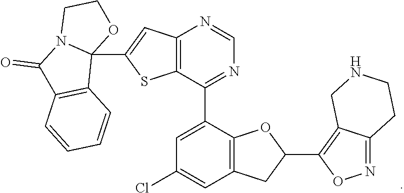 [SMILES: O=C1c2ccccc2C2(c3cc4ncnc(-c5cc(Cl)cc6c5OC(c5onc7c5CNCC7)C6)c4s3)OCCN12]

In embodiments, the compound has the formula:

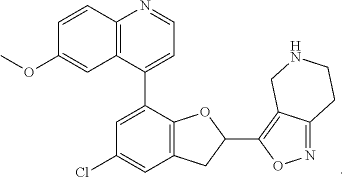 [SMILES: COc1ccc2nccc(-c3cc(Cl)cc4c3OC(c3onc5c3CNCC5)C4)c2c1]

In embodiments, the compound has the formula:

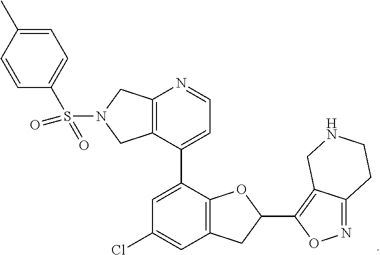 [SMILES: Cc1ccc(S(=O)(=O)N2Cc3nccc(-c4cc(Cl)cc5c4OC(c4onc6c4CNCC6)C5)c3C2)cc1]

In embodiments, the compound has the formula:

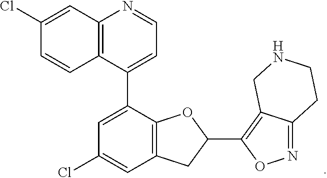 [SMILES: Clc1cc2c(c(-c3ccnc4cc(Cl)ccc34)c1)OC(c1onc3c1CNCC3)C2]

In embodiments, the compound has the formula:

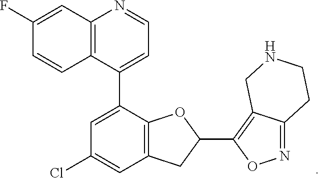 [SMILES: Fc1ccc2c(-c3cc(Cl)cc4c3OC(c3onc5c3CNCC5)C4)ccnc2c1]

In embodiments, the compound has the formula:

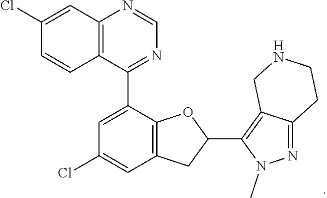 [SMILES: Cn1nc2c(c1C1Cc3cc(Cl)cc(-c4ncnc5cc(Cl)ccc45)c3O1)CNCC2]

In embodiments, the compound has the formula:

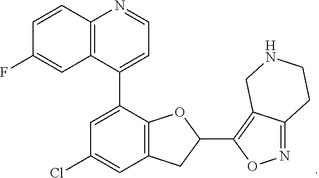 [SMILES: Fc1ccc2nccc(-c3cc(Cl)cc4c3OC(c3onc5c3CNCC5)C4)c2c1]

In embodiments, the compound has the formula:

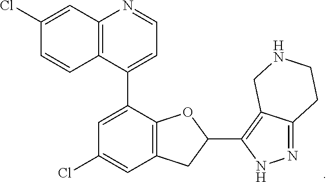 [SMILES: Clc1cc2c(c(-c3ccnc4cc(Cl)ccc34)c1)OC(c1[nH]nc3c1CNCC3)C2]

In embodiments, the compound has the formula:

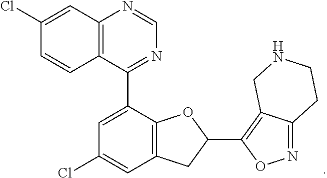 [SMILES: Clc1cc2c(c(-c3ncnc4cc(Cl)ccc34)c1)OC(c1onc3c1CNCC3)C2]

In embodiments, the compound has the formula:

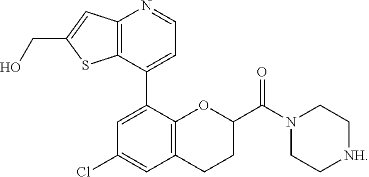 [SMILES: O=C(C1CCc2cc(Cl)cc(-c3ccnc4cc(CO)sc34)c2O1)N1CCNCC1]

In embodiments, the compound has the formula:

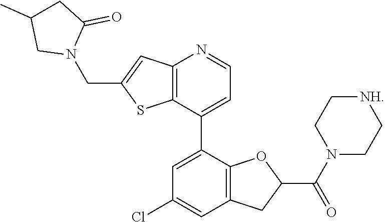 [SMILES: CC1CC(=O)N(Cc2cc3nccc(-c4cc(Cl)cc5c4OC(C(=O)N4CCNCC4)C5)c3s2)C1]

In embodiments, the compound has the formula:

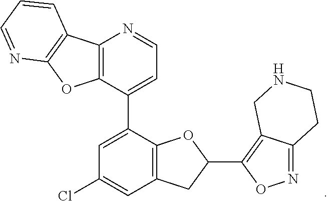 [SMILES: Clc1cc2c(c(-c3ccnc4c3oc3ncccc34)c1)OC(c1onc3c1CNCC3)C2]

In embodiments, the compound has the formula:

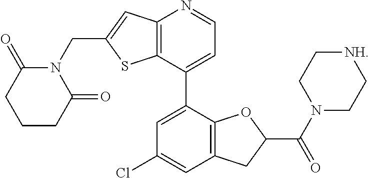 [SMILES: O=C(C1Cc2cc(Cl)cc(-c3ccnc4cc(CN5C(=O)CCCC5=O)sc34)c2O1)N1CCNCC1]

In embodiments, the compound has the formula:

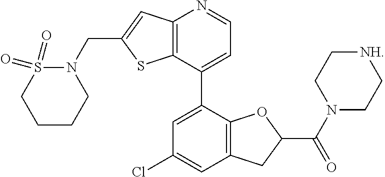 [SMILES: O=C(C1Cc2cc(Cl)cc(-c3ccnc4cc(CN5CCCCS5(=O)=O)sc34)c2O1)N1CCNCC1]

In embodiments, the compound has the formula:

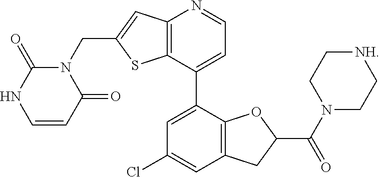 [SMILES: O=C(C1Cc2cc(Cl)cc(-c3ccnc4cc(Cn5c(=O)cc[nH]c5=O)sc34)c2O1)N1CCNCC1]

In embodiments, the compound has the formula:

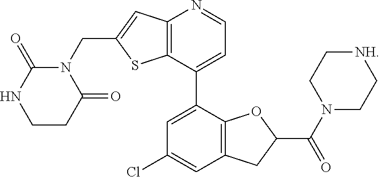 [SMILES: O=C(C1Cc2cc(Cl)cc(-c3ccnc4cc(CN5C(=O)CCNC5=O)sc34)c2O1)N1CCNCC1]

In embodiments, the compound has the formula:

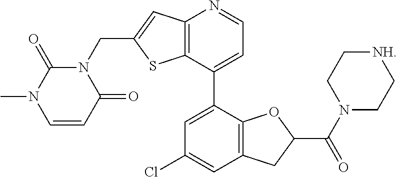 [SMILES: Cn1ccc(=O)n(Cc2cc3nccc(-c4cc(Cl)cc5c4OC(C(=O)N4CCNCC4)C5)c3s2)c1=O]

In embodiments, the compound has the formula as described elsewhere herein, for example within a table, figure, claim or example.

### III. PHARMACEUTICAL COMPOSITIONS

In an aspect, there is provided a pharmaceutical composition, including a compound as described herein, including embodiments (e.g., structural Formulae (I), (II), (III), (IV), (V), (VI), (VII), (VII-1A), (VII-2A), (VII-3A), (VIII), (VIII-1), (VIII-2), (VIII-3), (VIII-4), (VIII-5), (VIII-6), (VIII-7), (VIII-8), (VIII-9), (VIII-10), (VIII-11), (VIII-12), (IX), (X), (X-A1), (XI) or (XII)), and a pharmaceutically acceptable excipient.

The compounds as described herein of the present disclosure may be in the form of compositions suitable for administration to a subject. In general, such compositions are “pharmaceutical compositions” including a compound (e.g., compounds described herein) and one or more pharmaceutically acceptable or physiologically excipient (e.g., acceptable diluents or carriers). In certain embodiments, the compounds are present in a therapeutically acceptable amount. The pharmaceutical compositions may be used in the methods of the present disclosure; thus, for example, the pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein.

The pharmaceutical compositions containing the active ingredient (e.g., an inhibitor of USP7 function, or a compound described herein) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with nontoxic pharmaceutically acceptable excipients which are suitable for the manufacture thereof. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylenevinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly(methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavoring agents may be added to provide a palatable oral preparation.

Dispersible powders and granules suitable for preparation of an aqueous suspension by the addition of water provide the active ingredient in admixture with a dispersing or wetting agent, and optionally one or more suspending agents and/or preservatives. Suitable dispersing or wetting agents and suspending agents are exemplified herein.

The pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of a compound described herein contemplated by the present disclosure and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate-buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer; N-(2-Hydroxyethyl)piperazine-N′-(2-ethanesulfonic acid) (HEPES); 2-(N-Morpholino)ethanesulfonic acid (MES); 2-(N-Morpholino)ethanesulfonic acid sodium salt (MES); 3-(N-Morpholino)propanesulfonic acid (MOPS); and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time-delay material such as glyceryl monostearate or glyceryl stearate alone, or in combination with a wax, may be employed. Any drug delivery apparatus may be used to deliver a USP7 inhibitor, including implants (e.g., implantable pumps) and catheter systems, slow injection pumps and devices, all of which are well known to the skilled artisan.

Depot injections, which are generally administered subcutaneously or intramuscularly, may also be utilized to release a compound disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor® EL (BASF, Parsippany, N.J.) or phosphate buffered saline (PBS), ethanol, polyol (e.g., glycerol, propylene glycol, and liquid polyethylene glycol), and suitable mixtures thereof. In addition, sterile fixed oils are conventionally employed as a solvent or suspending medium; for this purpose, any bland fixed oil may be employed, including synthetic mono- or diglycerides. Moreover, fatty acids, such as oleic acid, find use in the preparation of injectables. Prolonged absorption of particular injectable formulations can be achieved by including an agent that delays absorption (e.g., aluminum monostearate or gelatin).

The present disclosure contemplates the administration of the compounds described herein in the form of suppositories for rectal administration. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The compounds described herein contemplated by the present disclosure may be in the form of any other suitable pharmaceutical composition (e.g., sprays for nasal or inhalation use) currently known or developed in the future.

### IV. METHODS OF USE

In another aspect, there is provided a method of inhibiting ubiquitin-specific-processing protease 7 (USP7), the method including contacting USP7 with a compound as described herein, including embodiments (e.g., structural Formulae (I), (II), (III), (IV), (V), (VI), (VII), (VII-1A), (VII-2A), (VII-3A), (VIII), (VIII-1), (VIII-2), (VIII-3), (VIII-4), (VIII-5), (VIII-6), (VIII-7), (VIII-8), (VIII-9), (VIII-10), (VIII-11), (VIII-12), (IX), (X), (X-A1), (XI) or (XII)), or a pharmaceutically acceptable salt thereof.

In an aspect, there is provided a method of treating or preventing a USP7-mediated disease or disorder, including administering to a patient in need thereof a therapeutically effective amount of a compound or pharmaceutical composition as described herein, including embodiments (e.g., structural Formulae (I), (II), (III), (IV), (V), (VI), (VII), (VII-1A), (VII-2A), (VII-3A), (VIII), (VIII-1), (VIII-2), (VIII-3), (VIII-4), (VIII-5), (VIII-6), (VIII-7), (VIII-8), (VIII-9), (VIII-10), (VIII-11), (VIII-12), (IX), (X), (X-A1), (XI) or (XII)), or a pharmaceutically acceptable salt thereof.

In embodiments, USP7-mediated disease or disorder is an autoimmunedisease, a cardiovascular disease, a viral infection, inflammatory disease (e.g., inflammation), a metabolism/endocrine disorder or a neurological disorder. In embodiments, USP7-mediated disease or disorder is an immune disorder. In embodiments, USP7-mediated disease or disorder is a cardiovascular disease. In embodiments, USP7-mediated disease or disorder is a viral infection. In embodiments, USP7-mediated disease or disorder is inflammatory disease (e.g., inflammation). In embodiments, USP7-mediated disease or disorder is a metabolism/endocrine disorder. In embodiments, USP7-mediated disease or disorder is a neurological disorder.

In embodiments, the USP7-mediated disease or disorder is cancer. In certain embodiments, the cancer includes, but is not limited to, breast cancer, ovarian cancer, cervical cancer, prostate cancer, cancer of the testes, genitourinary tract cancer, cancer of the esophagus, cancer of the larynx, glioblastoma, neuroblastoma, stomach cancer, skin cancer, keratoacanthoma, lung cancer, epidermoid carcinoma, large cell carcinoma, non-small cell lung carcinoma (NSCLC), small cell carcinoma, lung adenocarcinoma, bone cancer, colon cancer, adenoma, pancreatic cancer, adenocarcinoma, thyroid cancer, follicular carcinoma, undifferentiated carcinoma, papillary carcinoma, seminoma, melanoma, sarcoma, bladder carcinoma, liver carcinoma and biliary passages, kidney carcinoma, lymphoma, cancer of the pharynx, cancer of the lip, cancer of the tongue, cancer of the mouth, cancer of the small intestine, colorectal cancer, cancer of the large intestine, rectal cancer, brain cancer Hodgkin's, leukemia, cancer of the bronchus, cancer of the thyroid, liver cancer, intrahepatic bile duct cancer, gastric cancer, glioma/glioblastoma, endometrial cancer, melanoma, kidney cancer, renal cancer, pelvic cancer, urinary bladder cancer, multiple myeloma, acute myelogenous leukemia, chronic myelogenous leukemia, lymphocytic leukemia, chronic lymphoid leukemia (CLL), myeloid leukemia, cancer of the oral cavity and pharynx, non-Hodgkin lymphoma, melanoma or villous colon adenoma.

In another aspect, there is provided a method of treating a USP7-mediated cancer, including administering to a patient in need thereof a chemotherapeutic agent in combination with a therapeutically effective amount of a compound or pharmaceutical composition as described herein, including embodiments (e.g., structural Formulae (I), (II), (III), (IV), (V), (VI), (VII), (VII-1A), (VII-2A), (VII-3A), (VIII), (VIII-1), (VIII-2), (VIII-3), (VIII-4), (VIII-5), (VIII-6), (VIII-7), (VIII-8), (VIII-9), (VIII-10), (VIII-11), (VIII-12), (IX), (X), (X-A1), (XI) or (XII)), or a pharmaceutically acceptable salt thereof. In embodiments, the method further includes administering to the patient a chemotherapeutic agent in combination with a compound as described herein.

In embodiments, the disease or disorder is inflammatory bowel disease. In embodiments, the disease or disorder is rheumatoid arthritis. In embodiments, the disease or disorder is psoriasis. In embodiments, the disease or disorder includes allergy-related disorders (e.g., hypersensitivity and anaphylactic responses); gastrointestinal disorders (e.g., Crohn's disease, ulcerative colitis, ileitis and enteritis); psoriasis and inflammatory dermatoses (e.g., dermatitis, eczema, atopic dermatitis, allergic contact dermatitis, dermatomyositis, urticaria and pruritus); vasculitis; scleroderma; asthma, COPD, and respiratory allergic diseases (e.g., allergic rhinitis and hypersensitivity lung diseases); autoimmune diseases, including arthritis (e.g., rheumatoid and psoriatic), multiple sclerosis, systemic lupus erythematosus, type I diabetes and glomerulonephritis; graft rejection (e.g., allograft rejection); transplant rejection (e.g., solid organ); cancers, such as leukemias, lymphomas and metastatic cancers, particularly solid tumors (e.g., gastric cancers); and other diseases in which inhibition of undesired inflammatory and/or immune responses is desired, such as atherosclerosis, neurodegenerative diseases (e.g., Alzheimer's disease), encephalitis, meningitis, hepatitis, nephritis, sepsis, sarcoidosis, allergic conjunctivitis, otitis, and sinusitis. In particular embodiments, the USP7-mediated disease, disorder or condition is asthma, COPD, rhinitis, idiopathic pulmonary fibrosis, psoriasis and contact dermatitis. In embodiments the disease or disorder is pulmonary fibrosis, hepatic inflammation, asthma, atopic dermatitis, cancer (e.g., thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma), or granuloma development.

It is frequently beneficial to improve one of more physical properties of the treatment modalities disclosed herein and/or the manner in which they are administered. Improvements of physical properties include, for example, methods of increasing water solubility, bioavailability, serum half-life, and/or therapeutic half-life; and/or modulating biological activity. Modifications known in the art include pegylation, Fc-fusion and albumin fusion. Although generally associated with large molecule agents (e.g., polypeptides), such modifications have recently been evaluated with particular small molecules. By way of example, Chiang, M. et al. (J. Am. Chem. Soc., 2014, 136(9):3370-73) describe a small molecule agonist of the adenosine 2a receptor conjugated to the immunoglobulin Fc domain. The small molecule-Fc conjugate retained potent Fc receptor and adenosine 2a receptor interactions and showed superior properties compared to the unconjugated small molecule. Covalent attachment of PEG molecules to small molecule therapeutics has also been described (Li, W. et al., Progress in Polymer Science, 2013 38:421-44).

In embodiments, compounds of the present disclosure are effective in the treatment and prevention of IBD (e.g., Crohn's disease and ulcerative colitis, both of which are chronic idiopathic diseases that can affect any part of the gastrointestinal tract, and are associated with many untoward effects, and patients with prolonged ulcerative colitis are at an increased risk of developing colon cancer). Current IBD treatments are aimed at controlling inflammatory symptoms, and while certain agents (e.g., corticosteroids, aminosalicylates and standard immunosuppressive agents (e.g., cyclosporine, azathioprine, and methotrexate)) have met with limited success, long-term therapy may cause liver damage (e.g., fibrosis or cirrhosis) and bone marrow suppression, and patients often become refractory to such treatments.

The compounds of the present disclosure can be used to increase or enhance an immune response; to improve immunization, including increasing vaccine efficacy; and to increase inflammation. Immune deficiencies associated with immune deficiency diseases, immunosuppressive medical treatment, acute and/or chronic infection, and aging can be treated using the compounds disclosed herein. The compounds described herein can also be used to stimulate the immune system of patients suffering from iatrogenically-induced immune suppression, including those who have undergone bone marrow transplants, chemotherapy, or radiotherapy.

Oncology-related Disorders. In accordance with the present disclosure, a compound (e.g., a compound described herein) or pharmaceutical salt thereof can be used to treat or prevent a proliferative condition or disorder, including a cancer, for example, cancer of the uterus, cervix, breast, prostate, testes, gastrointestinal tract (e.g., esophagus, oropharynx, stomach, small or large intestines, colon, or rectum), kidney, renal cell, bladder, bone, bone marrow, skin, head or neck, liver, gall bladder, heart, lung, pancreas, salivary gland, adrenal gland, thyroid, brain (e.g., gliomas), ganglia, central nervous system (CNS) and peripheral nervous system (PNS), and cancers of the hematopoietic system and the immune system (e.g., spleen or thymus). The present disclosure also provides methods of treating or preventing other cancer-related diseases, disorders or conditions, including, for example, immunogenic tumors, non-immunogenic tumors, dormant tumors, virus-induced cancers (e.g., epithelial cell cancers, endothelial cell cancers, squamous cell carcinomas and papillomavirus), adenocarcinomas, lymphomas, carcinomas, melanomas, leukemias, myelomas, sarcomas, teratocarcinomas, chemically-induced cancers, metastasis, and angiogenesis. The disclosure contemplates reducing tolerance to a tumor cell or cancer cell antigen, e.g., by modulating activity of a regulatory T-cell and/or a CD8+ T-cell (see, e.g., Ramirez-Montagut, et al. (2003) Oncogene 22:3180-87; and Sawaya, et al. (2003) New Engl. J. Med. 349:1501-09). In some embodiments, the tumor or cancer is colon cancer, ovarian cancer, breast cancer, melanoma, lung cancer, glioblastoma, lymphoma, or leukemia. In particular embodiments, the cancer is gastric cancer. The use of the term(s) cancer-related diseases, disorders and conditions is meant to refer broadly to conditions that are associated, directly or indirectly, with cancer, and includes, e.g., angiogenesis and precancerous conditions such as dysplasia. In embodiments, the cancer is thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma.

In embodiments, a cancer be metastatic or at risk of becoming metastatic, or may occur in a diffuse tissue, including cancers of the blood or bone marrow (e.g., leukemia). In some further embodiments, the compounds of the disclosure can be used to overcome T-cell tolerance.

In some embodiments, the present disclosure provides methods for treating a proliferative condition, cancer, tumor, or precancerous condition with a compound described herein and at least one additional therapeutic or diagnostic agent, examples of which are set forth elsewhere herein.

The present disclosure provides methods for treating and/or preventing a proliferative condition, cancer, tumor, or precancerous disease, disorder or condition with a compound described herein.

In embodiments drawn to methods of treating cancer, the administration of a therapeutically effective amount of a compound described herein results in a cancer survival rate greater than the cancer survival rate observed by not administering a therapeutically effective amount of the compound. In further embodiments drawn to methods of treating cancer, the administration of a therapeutically effective amount of a compound described herein results in a reduction of tumor size or a slowing of tumor growth greater than reduction of tumor size or tumor growth observed following lack of administration of a therapeutically effective amount of the compound.

CNS-related and Neurological Disorders. Inhibition of USP7 activity may also represent an important strategy for the treatment or prevention of neurological, neuropsychiatric, neurodegenerative or other diseases, disorders and conditions having some association with the central nervous system, including disorders associated with impairment of cognitive function and/or motor function. Many of these diseases, disorders and conditions include an immune and/or inflammatory component. In embodiments, the disease or disorder is Parkinson's disease, extra pyramidal syndrome (EPS), dystonia, akathisia, tardive dyskinesia, restless leg syndrome, epilepsy, periodic limb movement in sleep, attention deficit disorders, depression, anxiety, dementia, Alzheimer's disease, Huntington's disease, multiple sclerosis, cerebral ischemia, hemorrhagic stroke, subarachnoid hemorrhage, or traumatic brain injury.

Other Disorders. Embodiments of the present disclosure contemplate the administration of the compounds described herein to a subject for the treatment or prevention of any other disorder that may benefit from at least some level of USP7 modulation. Such diseases, disorders and conditions may include, for example, asthma, chronic obstructive pulmonary disease (COPD) including chronic bronchitis and emphysema, idiopathic pulmonary fibrosis, atopic or contact dermatitis, urticaria, allergic rhinitis, nasal polyps, allergic conjunctivitis, thrombosis, reperfusion injury of the myocardium and brain, chronic glomerulonephritis, sepsis, adult respiratory distress syndrome, and pain. Additional diseases, disorders and conditions include allergic bronchopulmonary aspergillosis, allergic fungal sinusitis, severe asthma with fungal sensitization and diseases involving a pathogenic role for fungi including invasion or colonization (such as invasive aspergillosis, aspergilloma or candidiasis).

In embodiments, the disease or disorder includes cardiovascular (e.g., cardiac ischemia), metabolic (e.g., development of insulititis diabetes), hepatic (e.g., hepatic fibrosis, NASH, and NAFLD), ophthalmologic (e.g., diabetic retinopathy), or renal (e.g., renal failure) disorders.

The present disclosure contemplates the administration of the compounds described herein, and compositions (e.g., pharmaceutical salts, pharmaceutical composition) thereof, in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to release the compounds disclosed herein over a defined period of time. In embodiments, the administration is oral administration.

Metabolic and Cardiovascular Diseases. The present disclosure provides methods for treating and/or preventing certain cardiovascular- and/or metabolic-related diseases, disorders and conditions, as well as disorders associated therewith, with a compound described herein.

The compounds of the present disclosure may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED50 of an agent is the dose or amount of an agent that produces a therapeutic response or desired effect in 50% of the population to which it is administered. Although the ED50 is commonly used as a measure of reasonable expectance of an agent's effect, it is not necessarily the dose that a clinician might deem appropriate taking into consideration all relevant factors. Thus, in some situations the effective amount is more than the calculated ED50, in other situations the effective amount is less than the calculated ED50, and in still other situations the effective amount is the same as the calculated ED50.

In addition, an effective dose of the compounds of the present disclosure may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In embodiments, the compounds contemplated by the present disclosure may be administered (e.g., orally) at dosage levels of about 0.01 mg/kg to about 50 mg/kg, or about 1 mg/kg to about 25 mg/kg, of subject body weight per day, one, two, three, four or more times a day, to obtain the desired therapeutic effect. For administration of an oral agent, the compositions can be provided in the form of tablets, capsules and the like containing from 0.05 to 1000 milligrams of the active ingredient, particularly 0.05, 0.1, 0.25, 0.5, 0.75, 1.0, 1.25, 1.5, 1.75, 2.0, 2.5, 5.0, 7.5, 10.0, 15.0, 20.0, 25.0, 50.0, 75.0, 100.0, 125.0, 150.0, 175.0, 200.0, 250.0, 300.0, 400.0, 500.0, 600.0, 750.0, 800.0, 900.0, and 1000.0 milligrams of the active ingredient. A pharmaceutically acceptable carrier(s), diluent(s) and/or excipient(s) may be present in an amount of from about 0.1 g to about 2.0 g.

In embodiments, the dosage of the desired compound is contained in a “unit dosage form”. The phrase “unit dosage form” refers to physically discrete units, each unit including a predetermined amount of a compound (e.g., a compound described herein), sufficient to produce the desired effect. It will be appreciated that the parameters of a unit dosage form will depend on the particular agent and the effect to be achieved.

### V. KITS

In another aspect, provided herein is a kit including a compound described herein or pharmaceutical compositions thereof. The kits are generally in the form of a physical structure housing various components, as described below, and may be utilized, for example, in practicing the methods described above.

A kit may include one or more of the compounds disclosed herein (e.g., provided in a sterile container), which may be in the form of a pharmaceutical composition suitable for administration to a subject. In embodiments, the compound has the structure of Formulae (I), (II), (III), (IV), (V), (VI), (VII), (VII-1A), (VII-2A), (VII-3A), (VIII), (VIII-1), (VIII-2), (VIII-3), (VIII-4), (VIII-5), (VIII-6), (VIII-7), (VIII-8), (VIII-9), (VIII-10), (VIII-11), (VIII-12), (IX), (X), (X-A1), (XI) or (XII), including embodiments thereof, or a pharmaceutically acceptable salt thereof. The compounds described herein can be provided in a form that is ready for use (e.g., a tablet or capsule) or in a form requiring, for example, reconstitution or dilution (e.g., a powder) prior to administration. When the compound is in a form that needs to be reconstituted or diluted by a user, the kit may also include diluents (e.g., sterile water), buffers, pharmaceutically acceptable excipients, and the like, packaged with, or separately from, the compound. Each component of the kit may be enclosed within an individual container, and all of the various containers may be within a single package. A kit of the present disclosure may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### VI. EMBODIMENTS

### Embodiment P1

A compound of structural Formula (I):

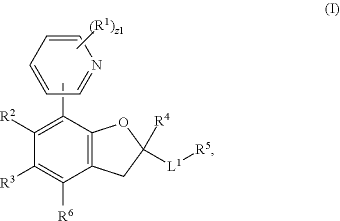

or a pharmaceutically acceptable salt thereof,

wherein:

n1, n2, n3, n4, n5 and n6 are independently an integer from 0 to 4;

m1, m2, m3, m4, m5, m6, v1, v2, v3, v4, v5 and v6 are independently 1 or 2;

z1 is an integer from 1 to 4;

L¹ is a bond, —O—, —S—, —NH—, —C(O)—, —C(O)NH, —C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R¹ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁H, —SOᵥ₁NH₂, —NHNH₂, —ONH₂, —NHC(O)NHNH₂, —NHC(O)NH₂, —N(O)ₘ₁, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R² is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂H, —SOᵥ₂NH₂, —NHNH₂, —ONH₂, —NHC(O)NHNH₂, —NHC(O)NH₂, —N(O)ₘ₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃H, —SOᵥ₃NH₂, —NHNH₂, —ONH₂, —NHC(O)NHNH₂, —NHC(O)NH₂, —N(O)ₘ₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R⁴ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₄H, —SOᵥ₄NH₂, —NHNH₂, —ONH₂, —NHC(O)NHNH₂, —NHC(O)NH₂, —N(O)ₘ₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R⁵ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; R⁶ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₆H, —SOᵥ₆NH₂, —NHNH₂, —ONH₂, —NHC(O)NHNH₂, —NHC(O)NH₂, —N(O)ₘ₆, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and R^{5A}, R^{5B}, R^{5C} and R^{5D} are independently hydrogen, halogen, —CF₃, —CCl₃, —CBr₃, —Cl₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{5B} and R^{5C} substituents bonded to the same nitrogen atom may optionally be joined to form a substituted or unsubstituted heterocycloalkyl or substituted or unsubstituted heteroaryl.

### Embodiment P2

The compound of embodiment P1, wherein the compound has structural Formula (II):

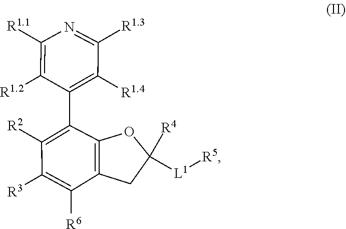

wherein:

n1.1, n1.2, n1.3 and n1.4 are independently and integer from 0 to 4; m1.1, m1.2, m1.3, m1.4, v1.1, v1.2, v1.3 and v1.4 are independently 1 or 2;

R^{1.1} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.1}R^{1.1A}, —SO_{v1.1}NR^{1.1B}R^{1.1C}, —NHNR^{1.1B}R^{11C}, —ONR^{1.1B}R^{1.1C}, —NHC(O)NHNR^{1.1B}R^{1.1C}, —NHC(O)NR^{1.1B}R^{11C}, —N(O)_{m1.1}, —NR^{1.1B}R^{1.1C}, —C(O)R^{1.1D}, —C(O)OR^{1.1D}, —C(O)NR^{1.1B}R^{1.1C}, —OR^{1.1A}, —NR^{1.1B}SO₂R^{1.1A}, —NR^{1.1B}C(O)R^{1.1D}, —NR^{1.1B}C(O)OR^{1.1D}, —NR^{1.1B}OR^{1.1D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; R^{1.2} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.2}H, —SO_{v1.2}NH₂, —NHNH₂, —ONH₂, —NHC(O)NHNH₂, —NHC(O)NH₂, —N(O)_{m1.2}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1.3} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.3}H, —SO_{v1.3}NH₂, —NHNH₂, —ONH₂, —NHC(O)NHNH₂, —NHC(O)NH₂, —N(O)_{m1.3}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1.4} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.4}H, —SO_{v1.4}NH₂, —NHNH₂, —ONH₂, —NHC(O)NHNH₂, —NHC(O)NH₂, —N(O)_{m1.4}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1.1A}, R^{1.1B}, R^{1.1C} and R^{1.1D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1.1}B and R^{1.1C} substituents bonded to the same nitrogen atom may optionally be joined to form a substituted or unsubstituted heterocycloalkyl or substituted or unsubstituted heteroaryl; and wherein R^{1.1} and R^{1.2} or R^{1.3} and R^{1.4} may optionally be joined to form a substituted or unsubstituted cycloalkyl, substituted or unsubstituted heterocycloalkyl, substituted or unsubstituted aryl or substituted or unsubstituted heteroaryl, and further wherein at least one of R^{1.1}, R^{1.2}, R^{1.3} and R^{1.4} is not hydrogen.

### Embodiment P3

The compound of embodiment P1 or P2, wherein L¹ is a bond or —C(O)—.

### Embodiment P4

The compound of embodiment P1 or P2, wherein R⁵ is substituted or unsubstituted cycloalkyl, substituted or unsubstituted heterocycloalkyl, substituted or unsubstituted aryl or substituted or unsubstituted heteroaryl.

### Embodiment P5

The compound of embodiment P1 or P2, wherein: L¹ is a bond; and R⁵ is a substituted or unsubstituted heterocycloalkyl or a substituted or unsubstituted heteroaryl.

### Embodiment P6

The compound of embodiment P1, P2 or P5, wherein R⁵ is a substituted or unsubstituted 5,6-fused ring system, wherein the 5 membered ring of the 5,6-fused ring system is covalently bound to L¹.

### Embodiment P7

The compound of any one of embodiments P1-P6, wherein R⁵ is:

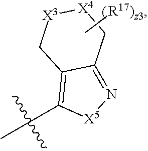

wherein: n12, n13, n14, n15, n16 and n17 are independently and integer from 0 to 4; m12, m13, m14, m15, m16, m17, v12, v13, v14, v15, v16 and v17 are independently 1 or 2;

z3 is an integer from 0 to 4;

X³ is C(R¹²)(R¹³) or N(R¹²); |

X⁴ is C(R¹⁴)(R¹⁵) or N(R¹⁴);

X⁵ is —O— or N(R¹⁶);

R¹² is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₂H, —SOᵥ₁₂NH₂, —NHNH₂, —ONH₂, —NHC(O)NHNH₂, —NHC(O)NH₂, —N(O)ₘ₁₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R¹³ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₃H, —SOᵥ₁₃NH₂, —NHNH₂, —ONH₂, —NHC(O)NHNH₂, —NHC(O)NH₂, —N(O)ₘ₁₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R¹⁴ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₄H, —SOᵥ₁₄NH₂, —NHNH₂, —ONH₂, —NHC(O)NHNH₂, —NHC(O)NH₂, —N(O)ₘ₁₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R¹⁵ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₅H, —SOᵥ₁₅NH₂, —NHNH₂, —ONH₂, —NHC(O)NHNH₂, —NHC(O)NH₂, —N(O)ₘₗ₅, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R¹⁶ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₁₆H, —SOᵥ₁₆NH₂, —NHNH₂, —ONH₂, —NHC(O)NHNH₂, —NHC(O)NH₂, —N(O)ₘ₁₆, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and

R¹⁷ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₁₇H, —SOᵥ₁₇NH₂, —NHNH₂, —ONH₂, —NHC(O)NHNH₂, —NHC(O)NH₂, —N(O)ₘₗ₇, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

### Embodiment P8

The compound of embodiment P7, wherein z3 is 0.

### Embodiment P9

The compound of embodiment P7 or P8, wherein: X³ is N(R¹²); and X⁴ is C(R¹⁴)(R¹⁵).

### Embodiment P10

The compound of any one of embodiments P7-P9, wherein R¹⁴ and R¹⁵ are hydrogen.

### Embodiment P11

The compound of embodiment P9 or P10, wherein R¹² is hydrogen.

### Embodiment P12

The compound of embodiment P7 or P8, wherein: X³ is C(R¹²)(R¹³); and X⁴ is N(R¹⁴).

### Embodiment P13

The compound of any one of embodiments P7-P8 or P12, wherein R¹² and R¹³ are hydrogen.

### Embodiment P14

The compound of embodiment P12 or P13, wherein R¹⁴ is hydrogen.

### Embodiment P15

The compound of any one of embodiments P7-P14, wherein X⁵ is —O—.

### Embodiment P16

The compound of any one of embodiments P7-P14, wherein X⁵ is N(R¹⁶).

### Embodiment P17

The compound of any one of embodiments P1-P6, wherein R⁵ is

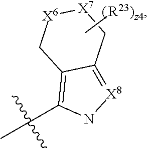

wherein:

n18, n19, n20, n21, n22 and n23 are independently and integer from 0 to 4;

m18, m19, m20, m21, m22, m23, v18, v19, v20, v21, v22 and v23 are independently 1 or 2;

z4 is an integer from 0 to 4;

X⁶ is C(R¹⁸)(R¹⁹) or N(R¹⁸);

X⁷ is C(R²⁰)(R²¹) or N(R²⁰);

X⁸ is —O— or N(R²²);

R¹⁸ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₈H, —SOᵥ₁₈NH₂, —NHNH₂, —ONH₂, —NHC(O)NHNH₂, —NHC(O)NH₂, —N(O)ₘ₁₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R¹⁹ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₉H, —SOᵥ₁₉NH₂, —NHNH₂, —ONH₂, —NHC(O)NHNH₂, —NHC(O)NH₂, —N(O)ₘ₁₉, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R²⁰ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₀H, —SOᵥ₂₀NH₂, —NHNH₂, —ONH₂, —NHC(O)NHNH₂, —NHC(O)NH₂, —N(O)ₘ₂₀, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R²¹ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₁H, —SOᵥ₂₁NH₂, —NHNH₂, —ONH₂, —NHC(O)NHNH₂, —NHC(O)NH₂, —N(O)ₘ₂₁, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R²² is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₂H, —SOᵥ₂₂NH₂, —NHNH₂, —ONH₂, —NHC(O)NHNH₂, —NHC(O)NH₂, —N(O)ₘ₂₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and

R²³ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₃H, —SOᵥ₂₃NH₂, —NHNH₂, —ONH₂, —NHC(O)NHNH₂, —NHC(O)NH₂, —N(O)ₘ₂₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

### Embodiment P18

The compound of embodiment P17, wherein z4 is 0.

### Embodiment P19

The compound of embodiment P17 or P18, wherein X⁶ is N(R¹⁸).

### Embodiment P20

The compound of any one of embodiments P17-P19, wherein X⁷ is C(R²⁰)(R²¹).

### Embodiment P21

The compound of any one of embodiments P17-P20, wherein R²⁰ is hydrogen.

### Embodiment P22

The compound of any one of embodiments P17-P20, wherein R²¹ is hydrogen.

### Embodiment P23

The compound of any one of embodiments P17-P20, wherein R¹⁸ is hydrogen.

### Embodiment P24

The compound of embodiment P17, wherein: X⁶ is N(R¹⁸); X⁷ is C(R²⁰)(R²¹); and R²⁰ and R²¹ are hydrogen.

### Embodiment P25

The compound of any one of embodiments P17-P24, wherein X⁸ is —O—.

### Embodiment P26

The compound of any one of embodiments P17-P24, wherein X⁸ is —N(R²²).

### Embodiment P27

The compound of any one of embodiments P1-P6, wherein R⁵ is:

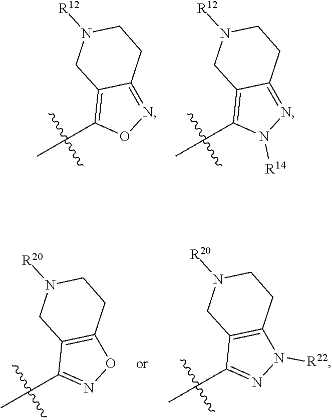

wherein:

n12, n14, n20 and n22 are independently and integer from 0 to 4;

m12, m14, m20, m22, v12, v14, v20 and v22 are independently 1 or 2; R¹² is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₂H, —SOᵥ₁₂NH₂, —NHNH₂, —ONH₂, —NHC(O)NHNH₂, —NHC(O)NH₂, —N(O)ₘ₁₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R¹⁴ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₄H, —SOᵥ₁₄NH₂, —NHNH₂, —ONH₂, —NHC(O)NHNH₂, —NHC(O)NH₂, —N(O)ₘ₁₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R²⁰ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₀H, —SOᵥ₂₀NH₂, —NHNH₂, —ONH₂, —NHC(O)NHNH₂, —NHC(O)NH₂, —N(O)ₘ₂₀, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and

R²² is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₂H, —SOᵥ₂₂NH₂, —NHNH₂, —ONH₂, —NHC(O)NHNH₂, —NHC(O)NH₂, —N(O)ₘ₂₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

### Embodiment P28

The compound of any one of embodiments P1-P6 or P27, wherein the compound has structural Formula (III):

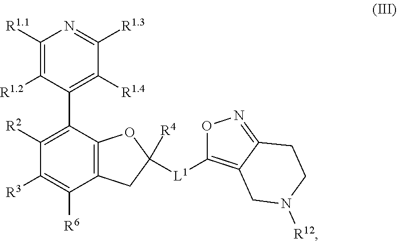

wherein:

n1.1, n1.2, n1.3, n1.4 and n12 are independently and integer from 0 to 4;

m1.1, m1.2, m1.3, m1.4, m12, v1.1, v1.2, v1.3, v1.4 and v12 are independently 1 or 2;

R^{1.1} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.1}R^{1.1A}, —SO_{v1.1}NR^{1.1B}R^{1.1C}, —NHNR^{1.1B}R^{1.1C}, —ONR^{1.1B}R^{1.1C}, —NHC(O)NHNR^{1.1B}R^{1.1C}, —NHC(O)NR^{1.1B}R^{1.1C}, —N(O)_{m1.1}, —NR^{1.1B}R^{1.1C}, —C(O)R^{1.1D}, —C(O)OR^{1.1D}, —C(O)NR^{1.1B}R^{1.1C}, —OR^{1.1A}, —NR^{1.1B}SO₂R^{1.1A}, —NR^{1.1B}C(O)R^{1.1D}, —NR^{1.1B}C(O)OR^{1.1D}, —NR^{1.1B}OR^{1.1D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R^{1.2} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.2}H, —SO_{v1.2}NH₂, —NHNH₂, —ONH₂, —NHC(O)NHNH₂, —NHC(O)NH₂, —N(O)_{m1.2}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1.3} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.3}H, —SO_{v1.3}NH₂, —NHNH₂, —ONH₂, —NHC(O)NHNH₂, —NHC(O)NH₂, —N(O)_{m1.3}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1.4} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.4}H, —SO_{v1.4}NH₂, —NHNH₂, —ONH₂, —NHC(O)NHNH₂, —NHC(O)NH₂, —N(O)_{m1.4}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹² is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₁₂H, —SOᵥ₁₂NH₂, —NHNH₂, —ONH₂, —NHC(O)NHNH₂, —NHC(O)NH₂, —N(O)ₘₗ₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R^{1.1A}, R^{1.1B}, R^{1.1C} and R^{1.1D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1.1B} and R^{1.1C} substituents bonded to the same nitrogen atom may optionally be joined to form a substituted or unsubstituted heterocycloalkyl or substituted or unsubstituted heteroaryl; and wherein R^{1.1} and R^{1.2} or R^{1.3} and R^{1.4} may optionally be joined to form a substituted or unsubstituted cycloalkyl, substituted or unsubstituted heterocycloalkyl, substituted or unsubstituted aryl or substituted or unsubstituted heteroaryl, and further wherein at least one of R^{1.1}, R^{1.2}, R^{1.3} and R^{1.4} is not hydrogen.

### Embodiment P29

The compound of embodiment P1 or P2, wherein the compound has structural Formula (IV):

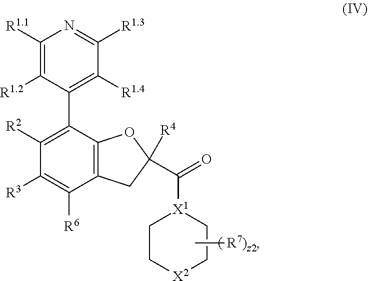

wherein:

X¹ is C(R⁸) or N;

X² is C(R⁹)(R¹⁰) or N(R⁹);

n1.1, n1.2, n1.3, n1.4, n7, n8, n9 and n10 are independently and integer from 0 to 4;

m1, m1.2, m1.3, m1.4, m7, m8, m9, m10, v1.1, v1.2, v1.3, v1.4, v7, v8, v9 and v10 are independently 1 or 2;

z2 is an integer from 0 to 8;

R^{1.1} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.1}R^{1.1A}, —SO_{v1.1}NR^{1.1B}R^{1.1C}, —NHNR^{1.1B}R^{1.1C}, —ONR^{1.1B}R^{1.1C}, —NHC(O)NHNR^{1.1B}R^{1.1C}, —NHC(O)NR^{1.1B}R^{1.1C}, —N(O)_{m1.1}, —NR^{1.1B}R^{1.1C}, —C(O)R^{1.1D}, —C(O)OR^{1.1D}, —C(O)NR^{1.1B}R^{1.1C}, —OR^{1.1A}, —NR^{1.1B}SO₂R^{1.1A}, —NR^{1.1B}C(O)R^{1.1D}, —NR^{1.1B}C(O)OR^{1.1D}, —NR^{1.1B}OR^{1.1D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R^{1.2} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.2}H, —SO_{v1.2}NH₂, —NHNH₂, —ONH₂, —NHC(O)NHNH₂, —NHC(O)NH₂, —N(O)_{m1.2}, —NH₂, —C(O)H, —C(O)OH, —C(O)NH₂, —OH, —NHSO₂H, —NHC(O)H, —NHC(O)OH, —NHOH—OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1.3} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.3}H, —SO_{v1.3}NH₂, —NHNH₂, —ONH₂, —NHC(O)NHNH₂, —NHC(O)NH₂, —N(O)_{m1.3}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1.4} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.4}H, —SO_{v1.4}NH₂, —NHNH₂, —ONH₂, —NHC(O)NHNH₂, —NHC(O)NH₂, —N(O)_{m1.4}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁷ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₇H, —SOᵥ₇NH₂, —NHNH₂, —ONH₂, —NHC(O)NHNH₂, —NHC(O)NH₂, —N(O)ₘ₇, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R⁸ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₅H, —SOᵥ₅NH₂, —NHNH₂, —ONH₂, —NHC(O)NHNH₂, —NHC(O)NH₂, —N(O)ₘ₅, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R⁹ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₉R^{9A}, —SOᵥ₉NR^{9B}R^{9C}, —NHNR^{9B}R^{9C}, —ONR^{9B}R^{9C}, —NHC(O)NHNR^{9B}R^{9C}, —NHC(O)NR^{9B}R^{9C}, —N(O)ₘ₉, —NR^{9B}R^{9C}, —C(O)R^{9D}, —C(O)OR^{9D}, —C(O)NR^{9B}R^{9C}, —OR^{9A}, —NR^{9B}SO₂R^{9A}, —NR^{9B}C(O)R^{9D}, —NR^{9B}C(O)OR^{9D}, —NR^{9B}OR^{9D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R¹⁰ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₀R^{10A}, —SOᵥ₁₀NR^{10B}R^{10C}, —NHNR^{10B}R^{10C}, —ONR^{10B}R^{10C}, —NHC(O)NHNR^{10B}R^{10C}, —NHC(O)NR^{10B}R^{1C}, —N(O)ₘ₁₀, —NR^{10B}R^{10C}, —C(O)R^{10D}, —C(O)OR^{10D}, —C(O)NR^{1B}R^{1C}, —OR^{10A}, —NR^{10B}SO₂R^{10A}, —NR^{10B}C(O)R^{10D}, —NR^{10B}C(O)OR^{10D}, —NR^{10B}OR^{10D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R^{1.1A}, R^{1.1B}, R^{1.1C}, R^{1.1D}, R^{9A}, R^{9B}, R^{9C}, R^{9A}, R^{10A}, R^{10B}, R^{10C} and R^{10D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1.1B} and R^{1.1C}, R^{9B} and R^{9C} or R^{10B} and R^{10C} substituents bonded to the same nitrogen atom may optionally be joined to form a substituted or unsubstituted heterocycloalkyl or substituted or unsubstituted heteroaryl; and wherein R^{1.1} and R^{1.2} or R^{1.3} and R^{1.4} may optionally be joined to form a substituted or unsubstituted cycloalkyl, substituted or unsubstituted heterocycloalkyl, substituted or unsubstituted aryl or substituted or unsubstituted heteroaryl, and further wherein at least one of R^{1.1}, R^{1.2}, R^{1.3} and R^{1.4} is not hydrogen.

### Embodiment P30

The compound of embodiment P29, wherein X¹ is N.

### Embodiment P31

The compound of embodiment P29 or P30, wherein X² is N(R⁹).

### Embodiment P32

The compound of embodiment P29 or P30, wherein X² is C(R⁹)(R¹⁰).

### Embodiment P33

The compound of embodiment P29, wherein X¹ is CR⁸.

### Embodiment P34

The compound of any one of embodiments P29-P33, wherein R⁹ is substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

### Embodiment P35

The compound of any one of embodiments P29-P33, wherein R⁹ is substituted or unsubstituted cycloalkyl, substituted or unsubstituted heterocycloalkyl, substituted or unsubstituted aryl or substituted or unsubstituted heteroaryl.

### Embodiment P36

The compound of any one of embodiments P29-P33, wherein X² is —NH.

### Embodiment P37

The compound of any one of embodiments P29-P33, wherein R⁹ is substituted or unsubstituted pyridine.

### Embodiment P38

The compound of any one of embodiments P2-P37, wherein R^{1.1} and R^{1.2} are joined together and taken with the pyridyl ring to which they are attached form:

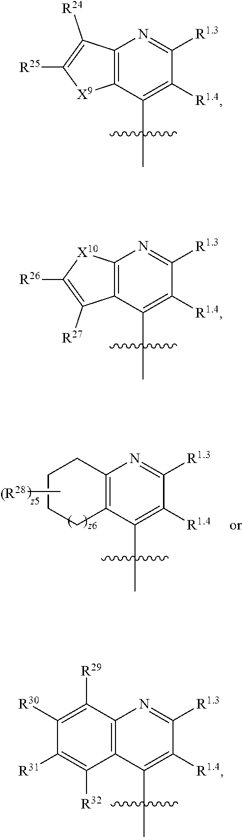

wherein:

z5 is an integer from 0 to 8;

z6 is 0 or 1;

n24, n25, n26, n27, n28, n29, n30, n31, n32, n33 and n34 are independently an integer from 0 to 4;

m24, m25, m26, m27, m28, m29, m30, m31, m32, m33, m34, v24, v25, v26, v27, v28, v29, v30, v31, v32, v33 and v34 are independently 1 or 2;

X⁹ is —O—, —S— or N(R³³);

X¹⁰ is —O—, —S— or N(R³⁴);

R²⁴ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₄H, —SOᵥ₂₄NH₂, —NHNH₂, —ONH₂, —NHC(O)NHNH₂, —NHC(O)NH₂, —N(O)ₘ₂₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁵ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₅R^{25A}, —SOᵥ₂₅NR^{25B}R^{25C}, —NHNR^{25B}R^{25C}, —ONR^{25B}R^{25C}, —NHC(O)NHNR^{25B}R^{25C}, —NHC(O)NR^{25B}R^{25C}, —N(O)ₘ₂₅, —NR^{25B}R^{25C}, —C(O)R^{25D}, —C(O)OR^{25D}, —C(O)NR^{25B}R^{25C}, —OR^{25A}, —NR^{25B}SO₂R^{25A}, —NR^{25B}C(O)R^{25D}, —NR^{25B}C(O)OR^{25D}, —NR^{25B}OR^{25D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R²⁶ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₆R^{26A}, —SOᵥ₂₆NR^{26B}R^{26C}, —NHNR^{26B}R^{26C}, —ONR^{26B}R^{26C}, —NC(O)NR^{26B}R^{26C}, —NHC(O)NR^{26B}R^{26C}, —N(O)ₘ₂₆, —NR^{26B}R^{26C}, —C(O)R^{26D}, —C(O)OR^{26D}, —C(O)NR^{26B}R^{26C}, —OR^{26A}, —NR^{26B}SO₂R^{26A}, —NR^{26B}C(O)R^{26D}, —NR^{26B}C(O)OR^{26D}, —NR^{26B}OR^{26D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R²⁷ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₇R^{27A}, —SOᵥ₂₇NR^{27B}R^{27C}, —NHNR^{27B}R^{27C}, —ONR^{27B}R^{27C}, —NHC(O)NHNR^{27B}R^{27C}, —NHC(O)NR^{27B}R^{27C}, —N(O)ₘ₂₇, —NR^{27B}R^{27C}, —C(O)R^{27D}, —C(O)OR^{27D}, —C(O)NR^{27B}R^{27C}, —OR^{27A}, —NR^{27B}SO₂R^{27A}, —NR^{27B}C(O)R^{27D}, —NR^{27B}C(O)OR^{27D}, —NR^{27B}OR^{27D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R²⁸ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₈H, —SOᵥ₂₈NH₂, —NHNH₂, —ONH₂, —NHC(O)NHNH₂, —NHC(O)NH₂, —N(O)ₘ₂₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁹ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₉H, —SOᵥ₂₉NH₂, —NHNH₂, —ONH₂, —NHC(O)NHNH₂, —NHC(O)NH₂, —N(O)ₘ₂₉, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³⁰ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃₀H, —SOᵥ₃₀NH₂, —NHNH₂, —ONH₂, —NHC(O)NHNH₂, —NHC(O)NH₂, —N(O)ₘ₃₀₀, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³¹ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃₁H, —SOᵥ₃₁NH₂, —NHNH₂, —ONH₂, —NHC(O)NHNH₂, —NHC(O)NH₂, —N(O)ₘ₃₁, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³² is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃₂H, —SOᵥ₃₂NH₂, —NHNH₂, —ONH₂, —NHC(O)NHNH₂, —NHC(O)NH₂, —N(O)ₘ₃₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³³ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃₃H, —SOᵥ₃₃NH₂, —NHNH₂, —ONH₂, —NHC(O)NHNH₂, —NHC(O)NH₂, —N(O)ₘ₃₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

R³⁴ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃₄H, —SOᵥ₃₄NH₂, —NHNH₂, —ONH₂, —NHC(O)NHNH₂, —NHC(O)NH₂, —N(O)ₘ₃₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{25A}, R^{25B}, R^{25C}, R^{25D}, R^{26A}, R^{26B}, R^{26C}, R^{26D}, R^{27A}, R^{27B}, R^{27C} and R^{27D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{26B} and R^{26C} or R^{27B} and R^{27C} substituents bonded to the same nitrogen atom may optionally be joined to form a substituted or unsubstituted heterocycloalkyl or substituted or unsubstituted heteroaryl.

### Embodiment P39

The compound of any one of embodiments P2-P38, wherein the compound has structural Formula (V):

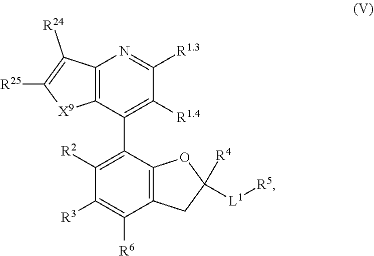

wherein:

n1.3, n1.4, n24 and n25 are independently and integer from 0 to 4;

m1.3, m1.4, m24, m25, v1.3, v1.4, v24 and v25 are independently 1 or 2;

X⁹ is —O— or —S—;

R^{1.3} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.3}H, —SO_{v1.3}NH₂, —NHNH₂, —ONH₂, —NHC(O)NHNH₂, —NHC(O)NH₂, —N(O)_{m1.3}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹⁴ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.4}H, —SO_{v1.4}NH₂, —NHNH₂, —ONH₂, —NHC(O)NHNH₂, —NHC(O)NH₂, —N(O)_{m1.4}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁴ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₄H, —SOᵥ₂₄NH₂, —NHNH₂, —ONH₂, —NHC(O)NHNH₂, —NHC(O)NH₂, —N(O)ₘ₂₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁵ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₅R^{25A}, —SOᵥ₂₅NR^{25B}R^{25C}, —NHNR^{25B}R^{25C}, —ONR^{25B}R^{25C}, —NHC(O)NHNR^{25B}R^{25C}, —NHC(O)NR^{25B}R^{25C}, —N(O)ₘ₂₅, —NR^{25B}R^{25C}, —C(O)R^{25D}, —C(O)OR^{25D}, —C(O)NR^{25B}R^{25C}, —OR^{25A}, —NR^{25B}SO₂R^{25A}, —NR^{25B}C(O)R^{25D}, —NR^{25B}C(O)OR^{25D}, —NR^{25B}OR^{25D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and R^{25A}, R^{25B}, R^{25C} and R^{25D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{25B} and R^{25C} substituents bonded to the same nitrogen atom may optionally be joined to form a substituted or unsubstituted heterocycloalkyl or substituted or unsubstituted heteroaryl.

### Embodiment P40

The compound of any one of embodiments P2-P38, wherein the compound has structural Formula (VI):

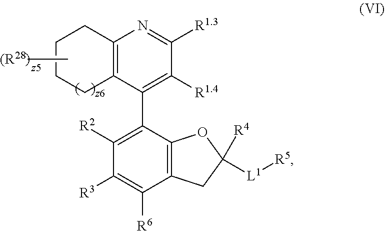

wherein:

z5 is an integer from 0 to 8;

z6 is 0 or 1;

n1.3, n1.4 and n28 are independently and integer from 0 to 4;

m1.3, m1.4, m28, v1.3, v1.4 and v28 are independently 1 or 2;

R^{1.3} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.3}H, —SO_{v1.3}NH₂, —NHNH₂, —ONH₂, —NHC(O)NHNH₂, —NHC(O)NH₂, —N(O)_{m1.3}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1.4} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.4}H, —SO_{v1.4}NH₂, —NHNH₂, —ONH₂, —NHC(O)NHNH₂, —NHC(O)NH₂, —N(O)_{m1.4}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

R²⁸ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₈H, —SOᵥ₂₈NH₂, —NHNH₂, —ONH₂, —NHC(O)NHNH₂, —NHC(O)NH₂, —N(O)ₘ₂₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment P41

The compound of embodiment P38 or P39, wherein R²⁴ and R²⁵ are independently hydrogen or —CN.

### Embodiment P42

The compound of embodiment P38 or P40, wherein R²⁶ and R²⁷ are independently hydrogen, substituted or unsubstituted heteroalkyl, substituted or unsubstituted cycloalkyl, substituted or unsubstituted heterocycloalkyl, substituted or unsubstituted aryl or substituted or unsubstituted heteroaryl.

### Embodiment P43

The compound of embodiment P38 or P40, wherein z5 is 0.

### Embodiment P44

The compound of embodiment P38, wherein R²⁹, R³⁰, R³¹ and R³² are independently —CH₂OH, —C(O)NH₂, —C(O)NHCH₃, —CH(OH)CH₂CH₃ or —CH(OH)CH₃.

### Embodiment P45

The compound of embodiment P1, wherein R¹ is hydrogen, halogen, —CN, substituted or unsubstituted alkyl or substituted or unsubstituted heteroalkyl.

### Embodiment P46

The compound of any one of embodiments P2-P37, wherein R¹¹, R^{1.2}, R^{1.3} and R^{1.4} are independently hydrogen, halogen, —CN, substituted or unsubstituted alkyl or substituted or unsubstituted heteroalkyl.

### Embodiment P47

The compound of any one of embodiments P1-P46, wherein R⁴ is hydrogen, substituted or unsubstituted alkyl or substituted or unsubstituted heteroalkyl.

### Embodiment P48

The compound of any one of embodiments P1-P46, wherein R⁴ is hydrogen or methyl.

### Embodiment P49

The compound of any one of embodiments P1-P46, wherein R² and R³ are independently hydrogen, halogen, substituted or unsubstituted alkyl or substituted or unsubstituted heteroalkyl.

### Embodiment P50

The compound of any one of embodiments P1-P46, wherein R² and R³ are methyl.

### Embodiment P51

The compound of any one of embodiments P1-P46, wherein R² is hydrogen.

### Embodiment P52

The compound of any one of embodiments P1-P46, wherein R³ is halogen, methyl or —CF₃.

### Embodiment P53

The compound of embodiment 1, wherein: L¹ is a bond or —C(O)—; two R¹ substituents bonded to adjacent carbon atoms are joined to form a 5-membered substituted or unsubstituted heteroaryl or a 6-membered substituted or unsubstituted aryl; R² and R³ are independently hydrogen or halogen; R⁴ is hydrogen or an unsubstituted alkyl; and R⁶ is hydrogen.

### Embodiment P54

The compound of embodiment P53, wherein R⁵ is

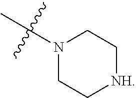 [SMILES: *N1CCNCC1]

### Embodiment P55

The compound of embodiment P1, wherein the compound is:

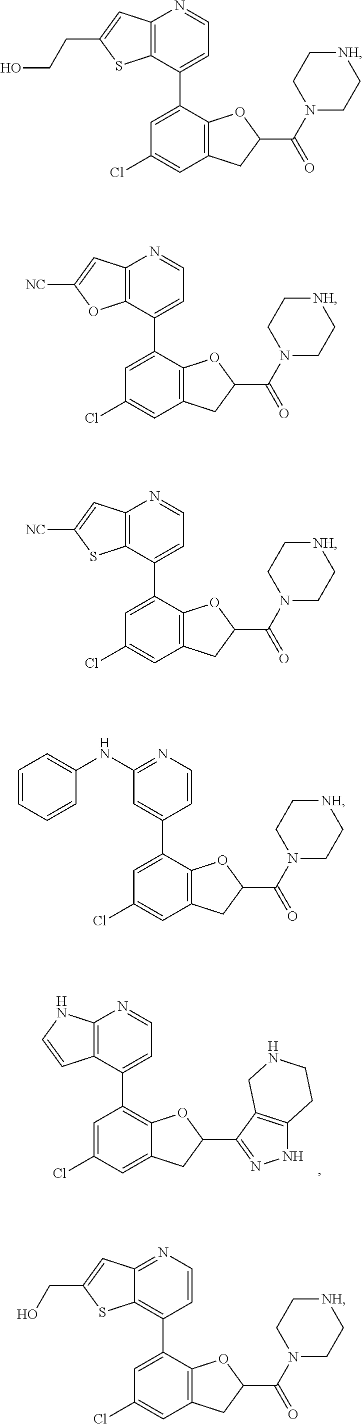 [SMILES: O=C(C1Cc2cc(Cl)cc(-c3ccnc4cc(CCO)sc34)c2O1)N1CCNCC1 ; N#Cc1cc2nccc(-c3cc(Cl)cc4c3OC(C(=O)N3CCNCC3)C4)c2o1 ; N#Cc1cc2nccc(-c3cc(Cl)cc4c3OC(C(=O)N3CCNCC3)C4)c2s1 ; O=C(C1Cc2cc(Cl)cc(-c3ccnc(Nc4ccccc4)c3)c2O1)N1CCNCC1 ; Clc1cc2c(c(-c3ccnc4[nH]ccc34)c1)OC(c1n[nH]c3c1CNCC3)C2 ; O=C(C1Cc2cc(Cl)cc(-c3ccnc4cc(CO)sc34)c2O1)N1CCNCC1]

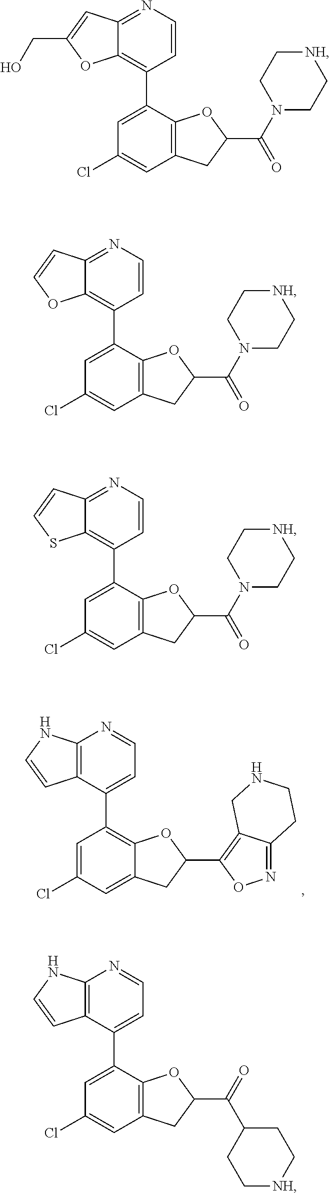 [SMILES: O=C(C1Cc2cc(Cl)cc(-c3ccnc4cc(CO)oc34)c2O1)N1CCNCC1 ; O=C(C1Cc2cc(Cl)cc(-c3ccnc4ccoc34)c2O1)N1CCNCC1 ; O=C(C1Cc2cc(Cl)cc(-c3ccnc4ccsc34)c2O1)N1CCNCC1 ; Clc1cc2c(c(-c3ccnc4[nH]ccc34)c1)OC(c1onc3c1CNCC3)C2 ; O=C(C1CCNCC1)C1Cc2cc(Cl)cc(-c3ccnc4[nH]ccc34)c2O1]

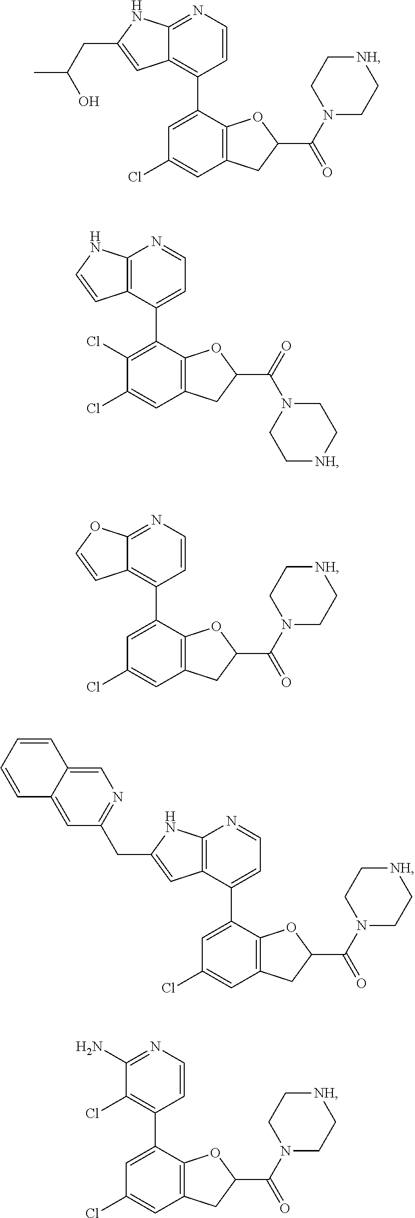 [SMILES: CC(O)Cc1cc2c(-c3cc(Cl)cc4c3OC(C(=O)N3CCNCC3)C4)ccnc2[nH]1 ; O=C(C1Cc2cc(Cl)c(Cl)c(-c3ccnc4[nH]ccc34)c2O1)N1CCNCC1 ; O=C(C1Cc2cc(Cl)cc(-c3ccnc4occc34)c2O1)N1CCNCC1 ; O=C(C1Cc2cc(Cl)cc(-c3ccnc4[nH]c(Cc5cc6ccccc6cn5)cc34)c2O1)N1CCNCC1 ; Nc1nccc(-c2cc(Cl)cc3c2OC(C(=O)N2CCNCC2)C3)c1Cl]

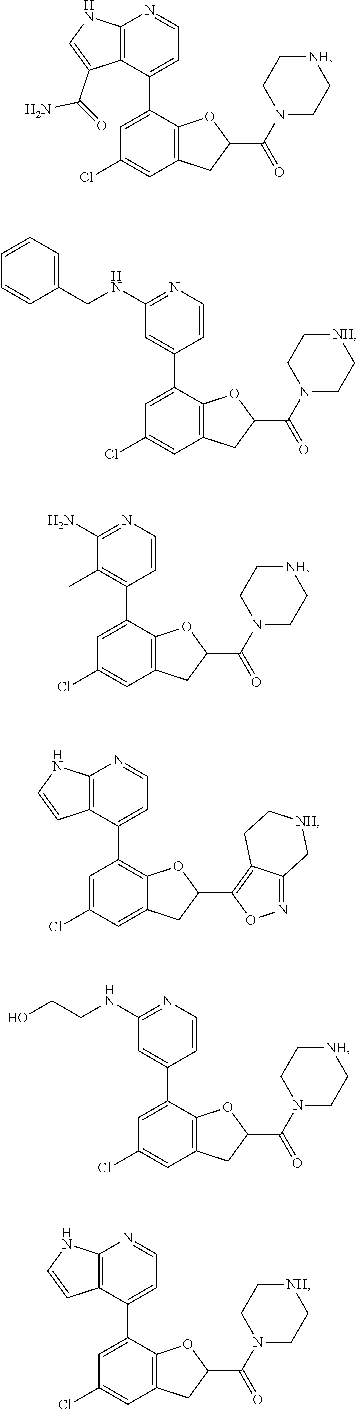 [SMILES: NC(=O)c1c[nH]c2nccc(-c3cc(Cl)cc4c3OC(C(=O)N3CCNCC3)C4)c12 ; O=C(C1Cc2cc(Cl)cc(-c3ccnc(NCc4ccccc4)c3)c2O1)N1CCNCC1 ; Cc1c(-c2cc(Cl)cc3c2OC(C(=O)N2CCNCC2)C3)ccnc1N ; Clc1cc2c(c(-c3ccnc4[nH]ccc34)c1)OC(c1onc3c1CCNC3)C2 ; O=C(C1Cc2cc(Cl)cc(-c3ccnc(NCCO)c3)c2O1)N1CCNCC1 ; O=C(C1Cc2cc(Cl)cc(-c3ccnc4[nH]ccc34)c2O1)N1CCNCC1]

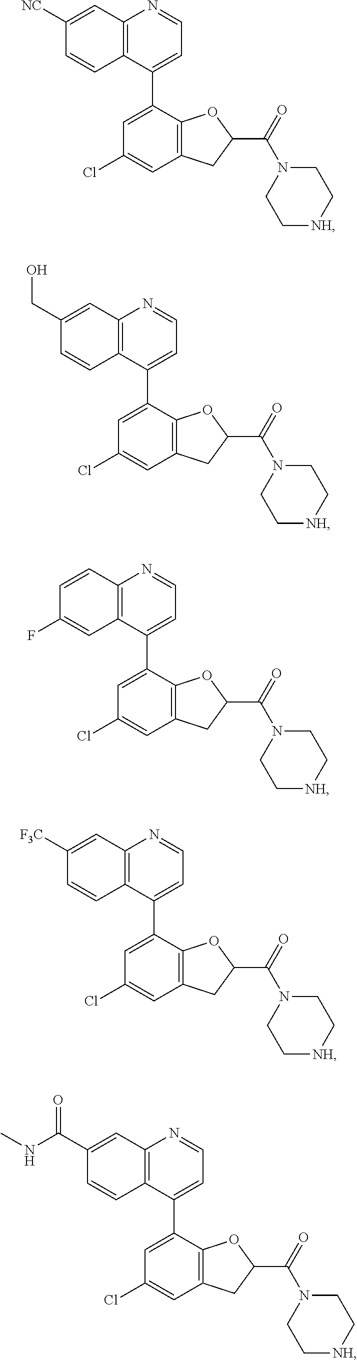 [SMILES: N#Cc1ccc2c(-c3cc(Cl)cc4c3OC(C(=O)N3CCNCC3)C4)ccnc2c1 ; O=C(C1Cc2cc(Cl)cc(-c3ccnc4cc(CO)ccc34)c2O1)N1CCNCC1 ; O=C(C1Cc2cc(Cl)cc(-c3ccnc4ccc(F)cc34)c2O1)N1CCNCC1 ; O=C(C1Cc2cc(Cl)cc(-c3ccnc4cc(C(F)(F)F)ccc34)c2O1)N1CCNCC1 ; CNC(=O)c1ccc2c(-c3cc(Cl)cc4c3OC(C(=O)N3CCNCC3)C4)ccnc2c1]

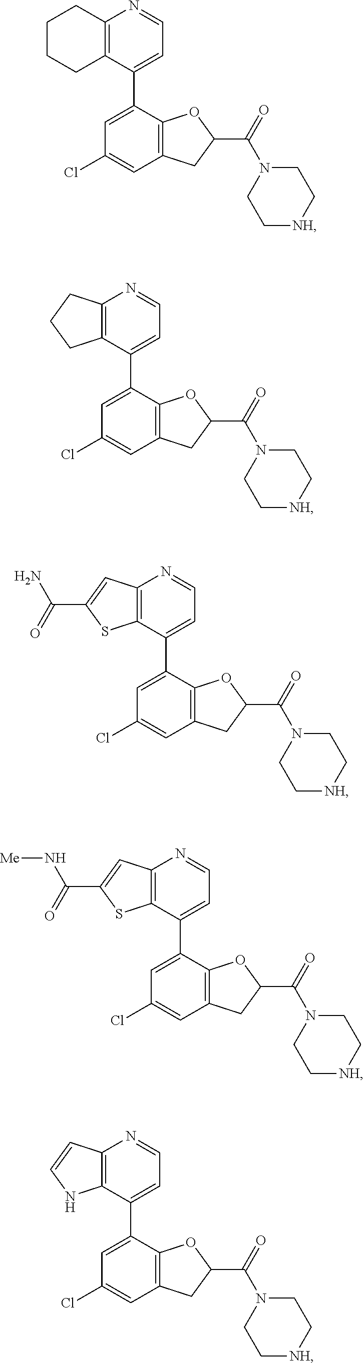 [SMILES: O=C(C1Cc2cc(Cl)cc(-c3ccnc4c3CCCC4)c2O1)N1CCNCC1 ; O=C(C1Cc2cc(Cl)cc(-c3ccnc4c3CCC4)c2O1)N1CCNCC1 ; NC(=O)c1cc2nccc(-c3cc(Cl)cc4c3OC(C(=O)N3CCNCC3)C4)c2s1 ; CNC(=O)c1cc2nccc(-c3cc(Cl)cc4c3OC(C(=O)N3CCNCC3)C4)c2s1 ; O=C(C1Cc2cc(Cl)cc(-c3ccnc4cc[nH]c34)c2O1)N1CCNCC1]

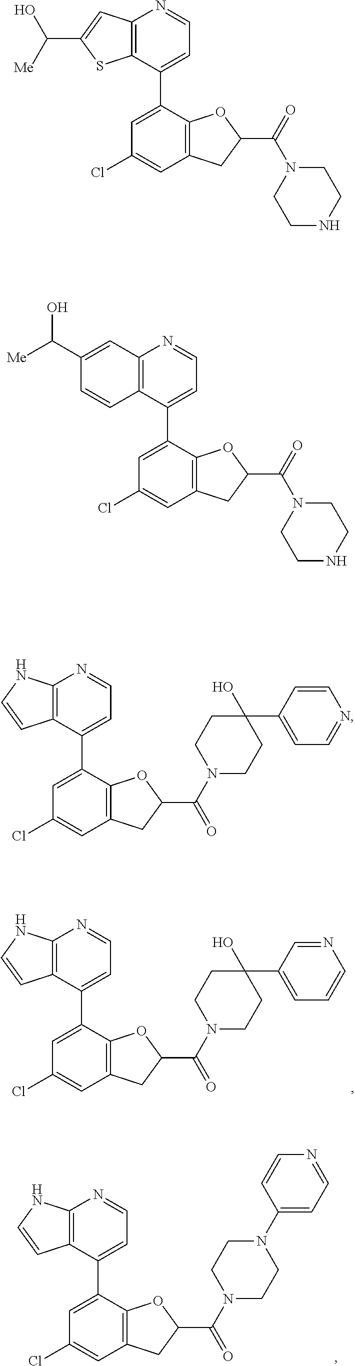 [SMILES: CC(O)c1cc2nccc(-c3cc(Cl)cc4c3OC(C(=O)N3CCNCC3)C4)c2s1 ; CC(O)c1ccc2c(-c3cc(Cl)cc4c3OC(C(=O)N3CCNCC3)C4)ccnc2c1 ; O=C(C1Cc2cc(Cl)cc(-c3ccnc4[nH]ccc34)c2O1)N1CCC(O)(c2ccncc2)CC1 ; O=C(C1Cc2cc(Cl)cc(-c3ccnc4[nH]ccc34)c2O1)N1CCC(O)(c2cccnc2)CC1 ; O=C(C1Cc2cc(Cl)cc(-c3ccnc4[nH]ccc34)c2O1)N1CCN(c2ccncc2)CC1]

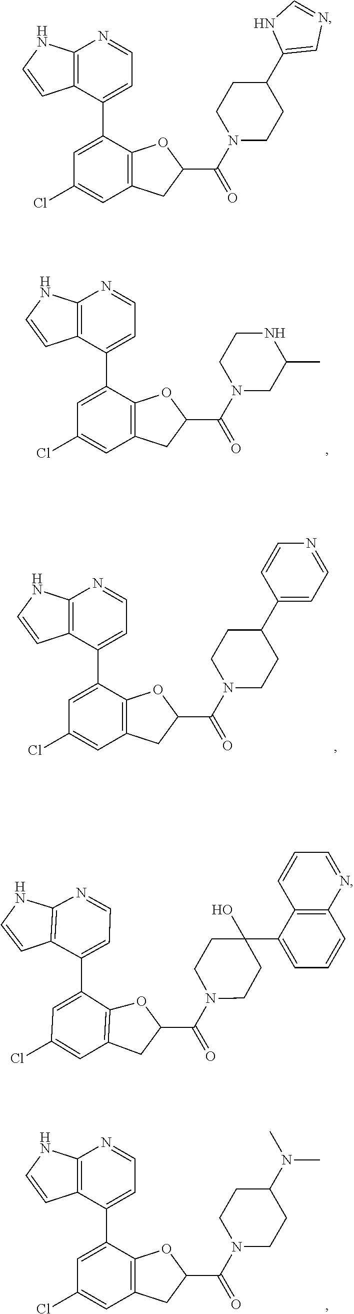 [SMILES: O=C(C1Cc2cc(Cl)cc(-c3ccnc4[nH]ccc34)c2O1)N1CCC(c2cnc[nH]2)CC1 ; CC1CN(C(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(c2ccncc2)CC1 ; O=C(C1Cc2cc(Cl)cc(-c3ccnc4[nH]ccc34)c2O1)N1CCC(O)(c2cccc3ncccc23)CC1 ; CN(C)C1CCN(C(=O)C2Cc3cc(Cl)cc(-c4ccnc5[nH]ccc45)c3O2)CC1]

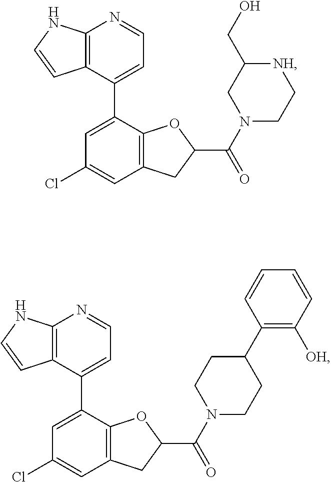 [SMILES: O=C(C1Cc2cc(Cl)cc(-c3ccnc4[nH]ccc34)c2O1)N1CCNC(CO)C1 ; O=C(C1Cc2cc(Cl)cc(-c3ccnc4[nH]ccc34)c2O1)N1CCC(c2ccccc2O)CC1]

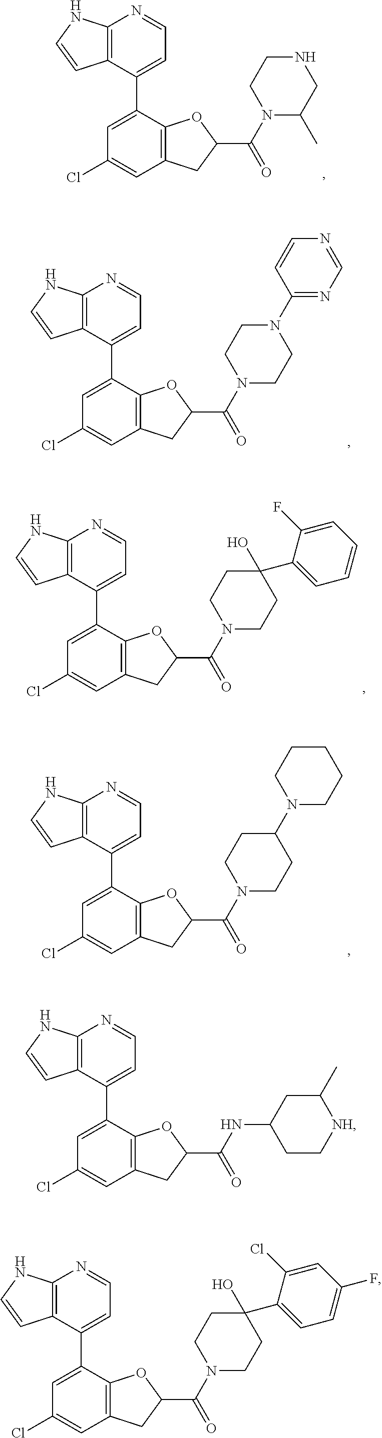 [SMILES: CC1CNCCN1C(=O)C1Cc2cc(Cl)cc(-c3ccnc4[nH]ccc34)c2O1 ; O=C(C1Cc2cc(Cl)cc(-c3ccnc4[nH]ccc34)c2O1)N1CCN(c2ccncn2)CC1 ; O=C(C1Cc2cc(Cl)cc(-c3ccnc4[nH]ccc34)c2O1)N1CCC(O)(c2ccccc2F)CC1 ; O=C(C1Cc2cc(Cl)cc(-c3ccnc4[nH]ccc34)c2O1)N1CCC(N2CCCCC2)CC1 ; CC1CC(NC(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(O)(c2ccc(F)cc2Cl)CC1]

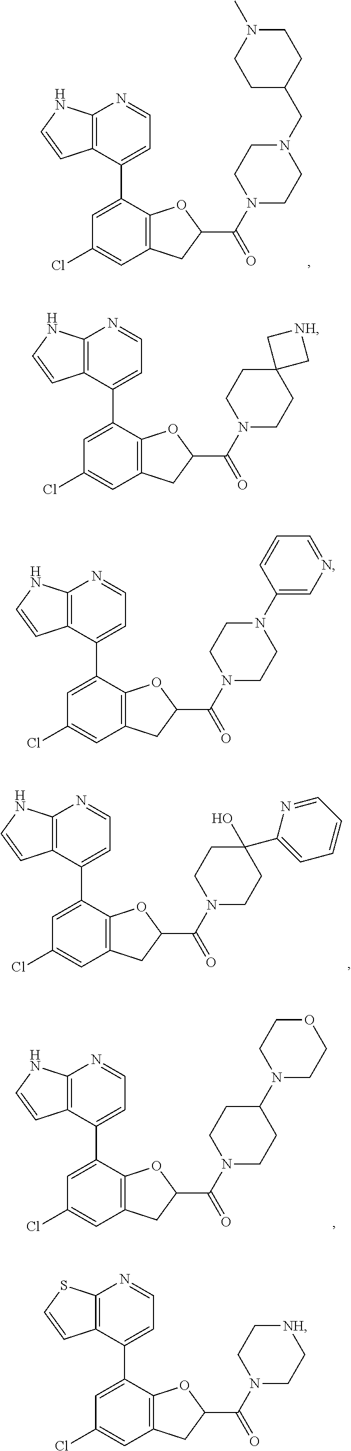 [SMILES: CN1CCC(CN2CCN(C(=O)C3Cc4cc(Cl)cc(-c5ccnc6[nH]ccc56)c4O3)CC2)CC1 ; O=C(C1Cc2cc(Cl)cc(-c3ccnc4[nH]ccc34)c2O1)N1CCC2(CC1)CNC2 ; O=C(C1Cc2cc(Cl)cc(-c3ccnc4[nH]ccc34)c2O1)N1CCN(c2cccnc2)CC1 ; O=C(C1Cc2cc(Cl)cc(-c3ccnc4[nH]ccc34)c2O1)N1CCC(O)(c2ccccn2)CC1 ; O=C(C1Cc2cc(Cl)cc(-c3ccnc4[nH]ccc34)c2O1)N1CCC(N2CCOCC2)CC1 ; O=C(C1Cc2cc(Cl)cc(-c3ccnc4sccc34)c2O1)N1CCNCC1]

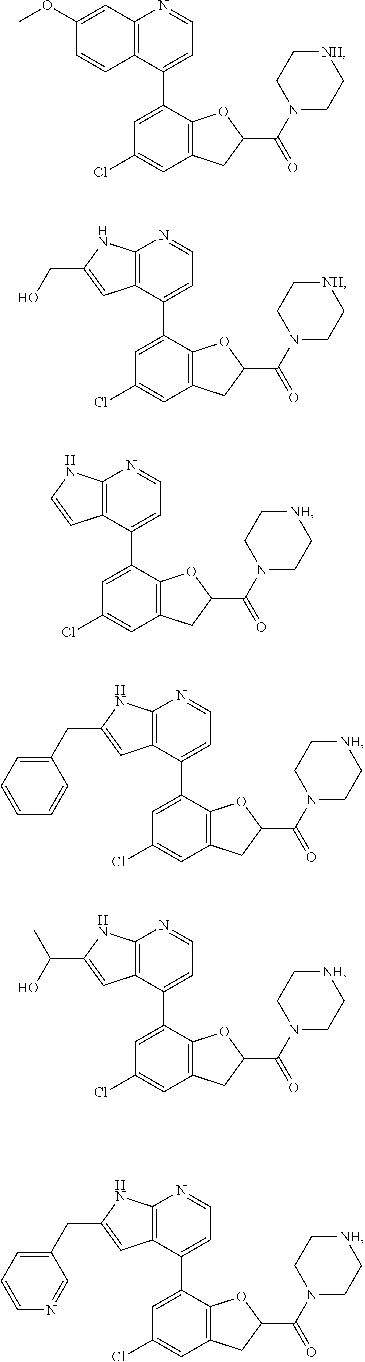 [SMILES: COc1ccc2c(-c3cc(Cl)cc4c3OC(C(=O)N3CCNCC3)C4)ccnc2c1 ; O=C(C1Cc2cc(Cl)cc(-c3ccnc4[nH]c(CO)cc34)c2O1)N1CCNCC1 ; O=C(C1Cc2cc(Cl)cc(-c3ccnc4[nH]ccc34)c2O1)N1CCNCC1 ; O=C(C1Cc2cc(Cl)cc(-c3ccnc4[nH]c(Cc5ccccc5)cc34)c2O1)N1CCNCC1 ; CC(O)c1cc2c(-c3cc(Cl)cc4c3OC(C(=O)N3CCNCC3)C4)ccnc2[nH]1 ; O=C(C1Cc2cc(Cl)cc(-c3ccnc4[nH]c(Cc5cccnc5)cc34)c2O1)N1CCNCC1]

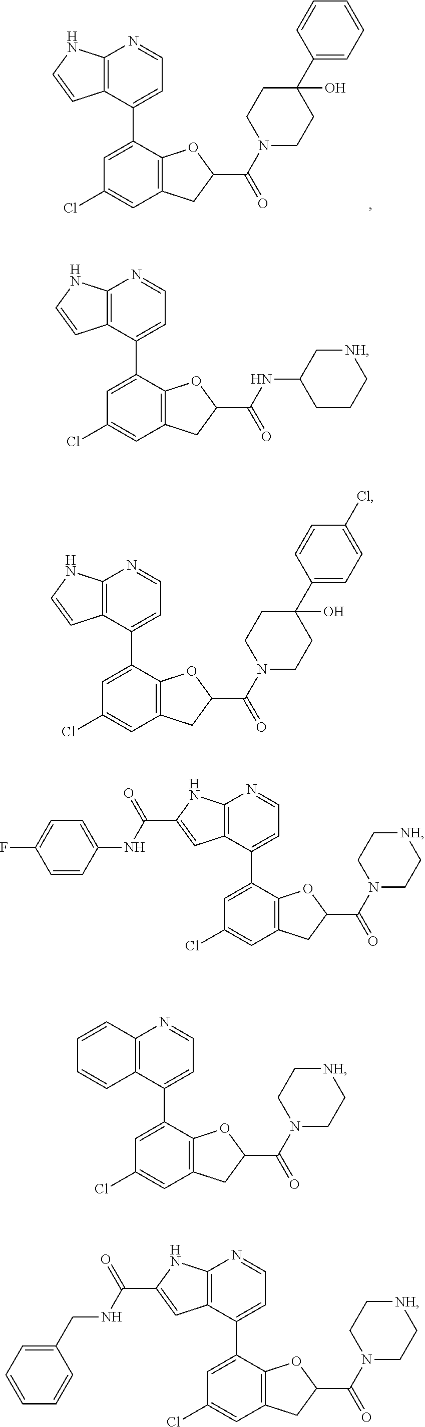 [SMILES: O=C(C1Cc2cc(Cl)cc(-c3ccnc4[nH]ccc34)c2O1)N1CCC(O)(c2ccccc2)CC1 ; O=C(NC1CCCNC1)C1Cc2cc(Cl)cc(-c3ccnc4[nH]ccc34)c2O1 ; O=C(C1Cc2cc(Cl)cc(-c3ccnc4[nH]ccc34)c2O1)N1CCC(O)(c2ccc(Cl)cc2)CC1 ; O=C(Nc1ccc(F)cc1)c1cc2c(-c3cc(Cl)cc4c3OC(C(=O)N3CCNCC3)C4)ccnc2[nH]1 ; O=C(C1Cc2cc(Cl)cc(-c3ccnc4ccccc34)c2O1)N1CCNCC1 ; O=C(NCc1ccccc1)c1cc2c(-c3cc(Cl)cc4c3OC(C(=O)N3CCNCC3)C4)ccnc2[nH]1]

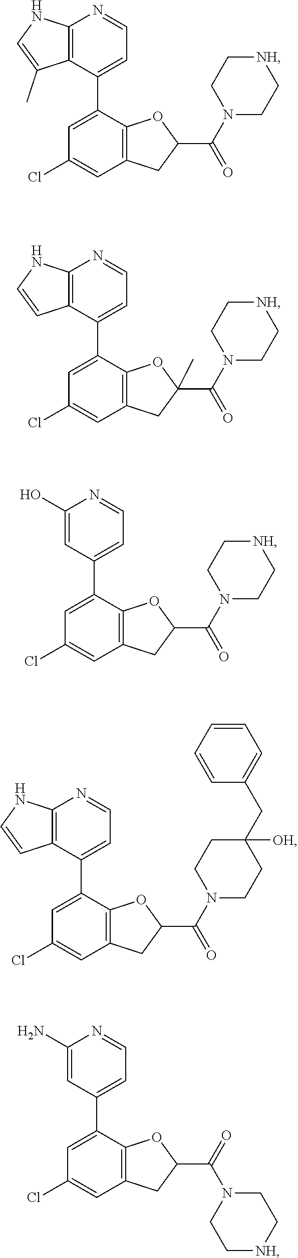 [SMILES: Cc1c[nH]c2nccc(-c3cc(Cl)cc4c3OC(C(=O)N3CCNCC3)C4)c12 ; CC1(C(=O)N2CCNCC2)Cc2cc(Cl)cc(-c3ccnc4[nH]ccc34)c2O1 ; O=C(C1Cc2cc(Cl)cc(-c3ccnc(O)c3)c2O1)N1CCNCC1 ; O=C(C1Cc2cc(Cl)cc(-c3ccnc4[nH]ccc34)c2O1)N1CCC(O)(Cc2ccccc2)CC1 ; Nc1cc(-c2cc(Cl)cc3c2OC(C(=O)N2CCNCC2)C3)ccn1]

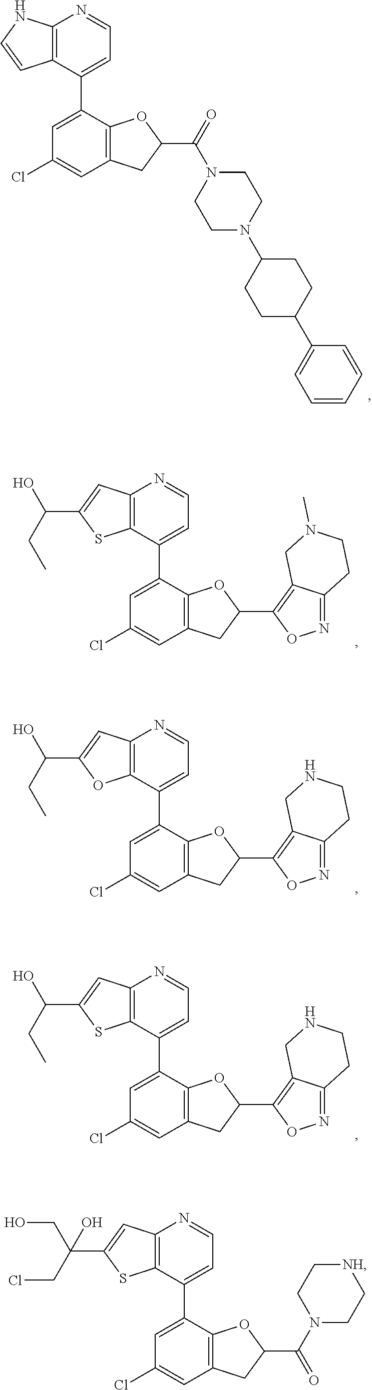 [SMILES: O=C(C1Cc2cc(Cl)cc(-c3ccnc4[nH]ccc34)c2O1)N1CCN(C2CCC(c3ccccc3)CC2)CC1 ; CCC(O)c1cc2nccc(-c3cc(Cl)cc4c3OC(c3onc5c3CN(C)CC5)C4)c2s1 ; CCC(O)c1cc2nccc(-c3cc(Cl)cc4c3OC(c3onc5c3CNCC5)C4)c2o1 ; CCC(O)c1cc2nccc(-c3cc(Cl)cc4c3OC(c3onc5c3CNCC5)C4)c2s1 ; O=C(C1Cc2cc(Cl)cc(-c3ccnc4cc(C(O)(CO)CCl)sc34)c2O1)N1CCNCC1]

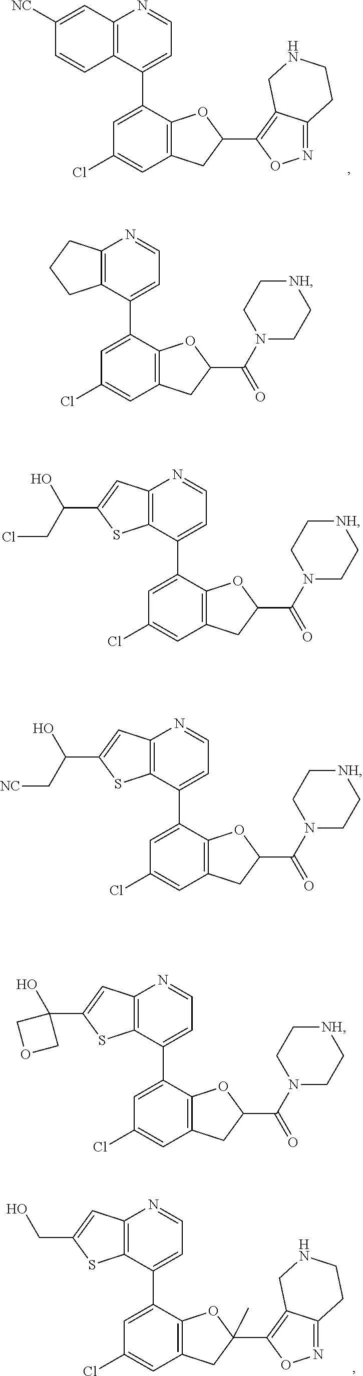 [SMILES: N#Cc1ccc2c(-c3cc(Cl)cc4c3OC(c3onc5c3CNCC5)C4)ccnc2c1 ; O=C(C1Cc2cc(Cl)cc(-c3ccnc4c3CCC4)c2O1)N1CCNCC1 ; O=C(C1Cc2cc(Cl)cc(-c3ccnc4cc(C(O)CCl)sc34)c2O1)N1CCNCC1 ; N#CCC(O)c1cc2nccc(-c3cc(Cl)cc4c3OC(C(=O)N3CCNCC3)C4)c2s1 ; O=C(C1Cc2cc(Cl)cc(-c3ccnc4cc(C5(O)COC5)sc34)c2O1)N1CCNCC1 ; CC1(c2onc3c2CNCC3)Cc2cc(Cl)cc(-c3ccnc4cc(CO)sc34)c2O1]

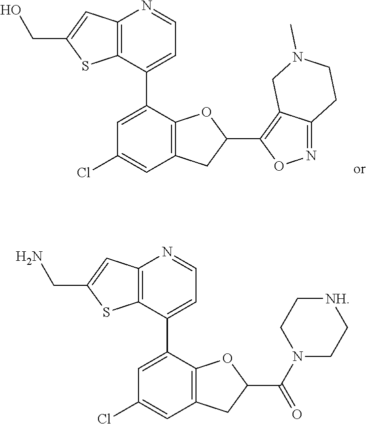 [SMILES: CN1CCc2noc(C3Cc4cc(Cl)cc(-c5ccnc6cc(CO)sc56)c4O3)c2C1 ; NCc1cc2nccc(-c3cc(Cl)cc4c3OC(C(=O)N3CCNCC3)C4)c2s1]

### Embodiment P56

A pharmaceutical composition, comprising the compound of any one of embodiments P1-P55 and a pharmaceutically acceptable excipient.

### Embodiment P57

A method of inhibiting ubiquitin-specific-processing protease 7 (USP7), comprising contacting USP7 with a compound of any one of embodiments P1-P55 or the pharmaceutical composition of embodiment P56.

### Embodiment P58

A method of treating a USP7-mediated disease or disorder, comprising administering to a patient in need thereof a therapeutically effective amount of a compound of any one of embodiments P1-P55 or the pharmaceutical composition of embodiment P56.

### Embodiment P59

The method of embodiment P58, wherein the USP7-mediated disease or disorder is an immune disorder, a cardiovascular disease, a viral infection, inflammation, a metabolism/endocrine disorder or a neurological disorder.

### Embodiment P60

The method of embodiment P58, wherein the USP7-mediated disease or disorder is cancer.

### Embodiment P61

The method of embodiment P60, wherein the cancer is breast cancer, ovary cancer, cervical cancer, prostate cancer, cancer of the testes, genitourinary tract cancer, cancer of the esophagus, cancer of the larynx, glioblastoma, neuroblastoma, stomach cancer, skin cancer, keratoacanthoma, lung cancer, epidermoid carcinoma, large cell carcinoma, non-small cell lung carcinoma (NSCLC), small cell carcinoma, lung adenocarcinoma, bone cancer, colon cancer, adenoma, pancreatic cancer, adenocarcinoma, thyroid cancer, follicular carcinoma, undifferentiated carcinoma, papillary carcinoma, seminoma, melanoma, sarcoma, bladder carcinoma, liver carcinoma and biliary passages, kidney carcinoma, lymphoma, cancer of the pharynx, cancer of the lip, cancer of the tongue, cancer of the mouth, cancer of the small intestine, colorectal cancer, cancer of the large intestine, rectal cancer, brain cancer Hodgkin's, leukemia, cancer of the bronchus, cancer of the thyroid, liver cancer, intrahepatic bile duct cancer, gastric cancer, glioma/glioblastoma, endometrial cancer, melanoma, kidney cancer, renal cancer, pelvic cancer, urinary bladder cancer, multiple myeloma, acute myelogenous leukemia, chronic myelogenous leukemia, lymphocytic leukemia, chronic lymphoid leukemia (CLL), myeloid leukemia, cancer of the oral cavity and pharynx, non-Hodgkin lymphoma, melanoma or villous colon adenoma.

### Embodiment P62

The method of embodiment P60 or P61, further comprising administering to the subject a chemotherapeutic agent in combination with the compound of embodiments P1-P55 or the pharmaceutical composition of embodiment P56.

### Embodiment P63

A compound of structural Formula (VII) or a pharmaceutically acceptable salt thereof:

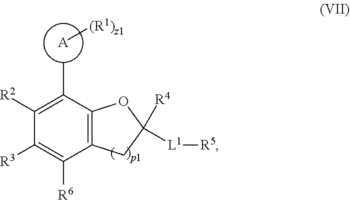

wherein: -Ring A-(R¹)_{z1} is

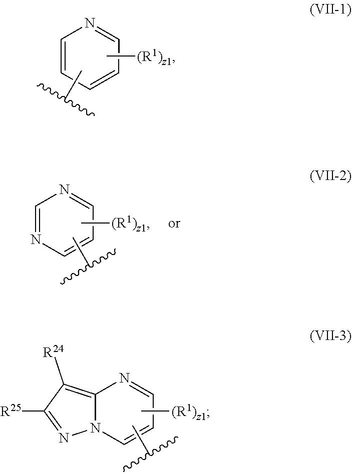

wherein,

p1 is 1 or 2;

L¹ is a bond, —O—, —S—, —NH—, —C(O)—, —C(O)NH, —C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R¹ is independently halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein two R¹ substituents bonded to adjacent carbon atoms may optionally be joined to form a substituted or unsubstituted cycloalkyl, substituted or unsubstituted heterocycloalkyl, substituted or unsubstituted aryl, or substituted or unsubstituted heteroaryl;

R² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₂H, —SOᵥ₂NH₂, —NHNH₂, —ONH₂, —NHC(O)NHNH₂, —NHC(O)NH₂, —N(O)ₘ₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₃H, —SOᵥ₃NH₂, —NHNH₂, —ONH₂, —NHC(O)NHNH₂, —NHC(O)NH₂, —N(O)ₘ₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₄H, —SOᵥ₄NH₂, —NHNH₂, —ONH₂, —NHC(O)NHNH₂, —NHC(O)NH₂, —N(O)ₘ₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁵ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁶ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₆H, —SOᵥ₆NH₂, —NHNH₂, —ONH₂, —NHC(O)NHNH₂, —NHC(O)NH₂, —N(O)ₘ₆, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, |

—OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₄H, —SOᵥ₂₄NH₂, —NHNH₂, —ONH₂, —NHC(O)NHNH₂, —NHC(O)NH₂, —N(O)ₘ₂₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁵ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₅R^{25A}, —SOᵥ₂₅NR^{25B}R^{25C}, —NHNR^{25B}R^{25C}, —ONR^{25B}R^{25C}, —NHC(O)NHNR^{25B}R^{25C}, —NHC(O)NR^{25B}R^{25C}, —N(O)ₘ₂₅, —NR^{25B}R^{25C}, —C(O)R^{25D}, —C(O)OR^{25D}, —C(O)NR^{25B}R^{25C}, —OR^{25A}, —NR^{25B}SO₂R^{25A}—NR^{25B}C(O)R^{25D}, —NR^{25B}C(O)OR^{25D}, —NR^{25B}OR^{25D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R^{1A}, R^{1B}, R^{1C}, R^{1D}, R^{5A}, R^{5B}, R^{5C}, R^{5D}, R^{25A}, R^{25B}, R^{25C}, and R^{25D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C}, R^{5B} and R^{5C}, or R^{25B} and R^{25C} substituents bonded to the same nitrogen atom may optionally be joined to form a substituted or unsubstituted heterocycloalkyl or substituted or unsubstituted heteroaryl;

n1, n2, n3, n4, n5, n6, n24, and n25 are independently an integer from 0 to 4;

m1, m2, m3, m4, m5, m6, m24, m25, v1, v2, v3, v4, v5, v6, v24, and v25 are independently 1 or 2; and

z1 is an integer from 0 to 4.

### Embodiment P64

The compound of embodiment P63, or a pharmaceutically acceptable salt thereof, wherein:

-Ring A-(R¹)_{z1} is:

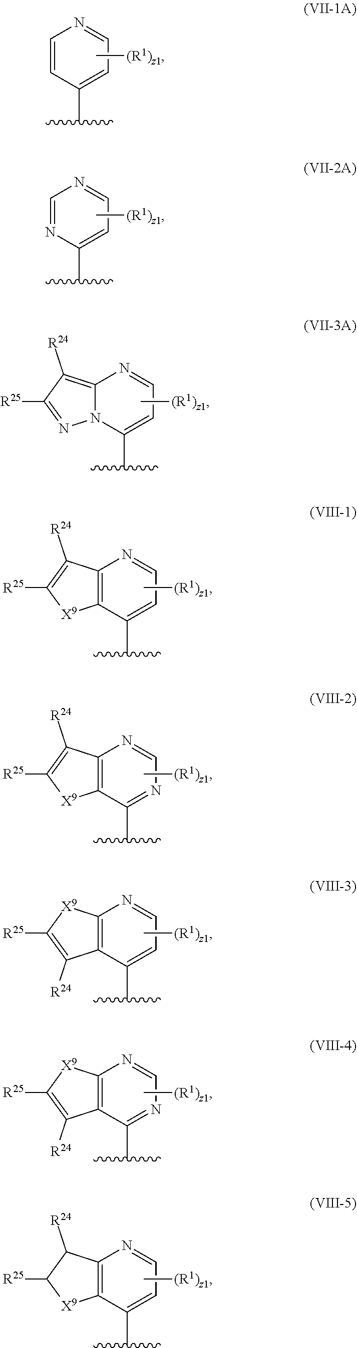

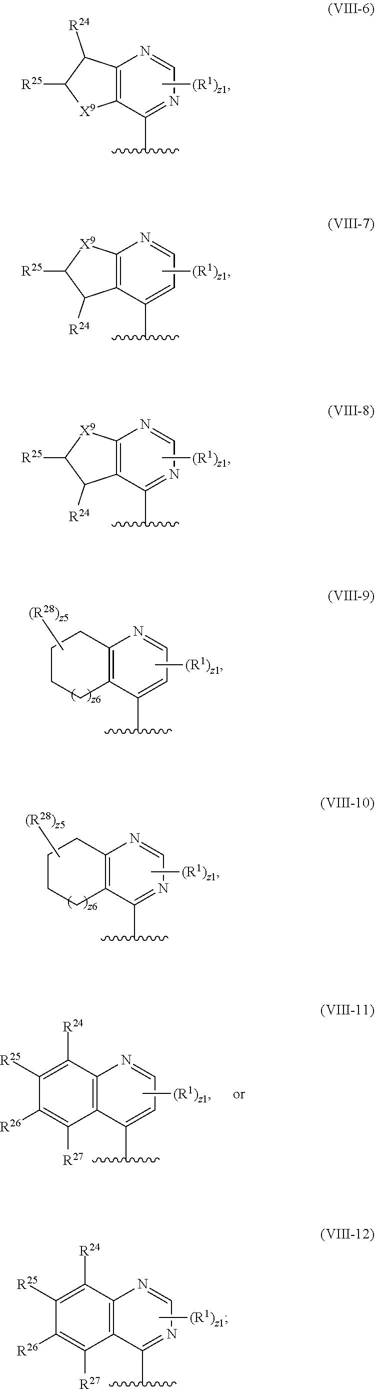

X⁹ is —O—, —S— or —N(R³³)—;

R²⁶ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₆R^{26A}, —SOᵥ₂₆NR^{26B}R^{26C}, —NHNR^{26B}R^{26C}, —ONR^{26B}R^{26C}, —NC(O)NR^{26B}R^{26C}, —NHC(O)NR^{26B}R^{26C}, —N(O)ₘ₂₆, —NR^{26B}R^{26C}, —C(O)R^{26D}, —C(O)OR^{26D}, —C(O)NR^{26B}R^{26C}, —OR^{26A}, —NR^{26B}SO₂R^{26A}, —NR²⁶BC(O)R^{26D}, —NR^{26B}C(O)OR^{26D}, —NR^{26B}OR^{26D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R²⁷ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₇R^{27A}, —SOᵥ₂₇NR^{27B}R^{27C}, —NHNR^{27B}R^{27C}, —ONR^{27B}R^{27C}, —NHC(O)NHNR^{27B}R^{27C}, —NHC(O)NR^{27B}R^{27C}, —N(O)ₘ₂₇, —NR^{27B}R^{27C}, —C(O)R^{27D}, —C(O)OR^{27D}, —C(O)NR^{27B}R^{27C}, —OR^{27A}, —NR^{27B}SO₂R^{27A}, —NR^{27B}C(O)R^{27D}, —NR^{27B}C(O)OR^{27D}, —NR^{27B}OR^{27D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R²⁸ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₈H, —SOᵥ₂₈NH₂, —NHNH₂, —ONH₂, —NHC(O)NHNH₂, —NHC(O)NH₂, —N(O)ₘ₂₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³³ is independently hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃₃H, —SOᵥ₃₃NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{26A}, R^{26B}, R^{26C}, R^{26D}, R^{27A}, R^{27B}, R^{27C}, and R^{27D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{26B} and R^{26C}, or R^{27B} and R^{27C} substituents bonded to the same nitrogen atom may optionally be joined to form a substituted or unsubstituted heterocycloalkyl or substituted or unsubstituted heteroaryl;

z5 is an integer from 0 to 8;

z6 is 0 or 1;

n26, n27, n28, and n33 are independently an integer from 0 to 4; and

m26, m27, m28, m33, v26, v27, v28, and v33 are independently 1 or 2.

### Embodiment P65

The compound of embodiment P64 or P64, or a pharmaceutically acceptable salt thereof, wherein the compound has structural Formula (IX):

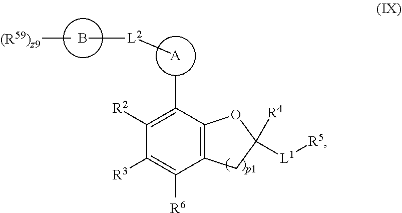

wherein:

Ring B is substituted or unsubstituted cycloalkyl, substituted or unsubstituted heterocycloalkyl, substituted or unsubstituted aryl, or substituted or unsubstituted heteroaryl;

L² is a bond, —O—, —S—, —NH—, —C(O)—, —C(O)NH, —C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and

R⁵⁹ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₅₉H, —SOᵥ₅₉NH₂, —NHNH₂, —ONH₂, —NHC(O)NHNH₂, —NHC(O)NH₂, —N(O)ₘ₅₉, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein two R⁵⁹ substituents bonded to adjacent atoms may optionally be joined to form a substituted or unsubstituted cycloalkyl, substituted or unsubstituted heterocycloalkyl, substituted or unsubstituted aryl, or substituted or unsubstituted heteroaryl;

n59 is an integer from 0 to 4;

m59 and v59 are independently 1 or 2; and

z9 is an integer from 0 to 6.

### Embodiment P66

The compound of any one of embodiments P63-P65, or a pharmaceutically acceptable salt thereof, wherein L¹ is a bond or —C(O)—.

### Embodiment P67

The compound of any one of embodiments P63-P66, or a pharmaceutically acceptable salt thereof, wherein R⁵ is substituted or unsubstituted cycloalkyl, substituted or unsubstituted heterocycloalkyl, substituted or unsubstituted aryl, or substituted or unsubstituted heteroaryl.

### Embodiment P68

The compound of any one of embodiments P63-P67, or a pharmaceutically acceptable salt thereof, wherein:

L¹ is a bond; and

R⁵ is a substituted or unsubstituted heterocycloalkyl or a substituted or unsubstituted heteroaryl.

### Embodiment P69

The compound of any one of embodiments P63-P68, or a pharmaceutically acceptable salt thereof, wherein R⁵ is a substituted or unsubstituted 5,6-fused ring system, wherein the 5-membered ring of the 5,6-fused ring system is covalently bound to L¹.

### Embodiment P70

The compound of embodiment P63 or P64, or a pharmaceutically acceptable salt thereof, wherein the compound has structural Formula (X):

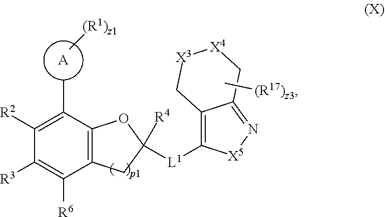

wherein:

X³ is

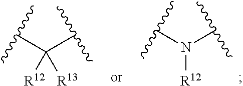

X⁴ is

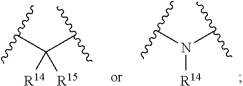

X⁵ is —O— or

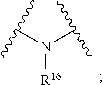

R¹² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —N₃, —CN, —SO₂CH₃, —SOₙ₁₂H, —SOᵥ₁₂NH₂, —NHNH₂, —ONH₂, —NHC(O)NHNH₂, —NHC(O)NH₂, —N(O)ₘ₁₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹³ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₃H, —SOᵥ₁₃NH₂, —NHNH₂, —ONH₂, —NHC(O)NHNH₂, —NHC(O)NH₂, —N(O)ₘ₁₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹² and R¹³ are optionally together oxo;

R¹⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₄H, —SOᵥ₁₄NH₂, —NHNH₂, —ONH₂, —NHC(O)NHNH₂, —NHC(O)NH₂, —N(O)ₘ₁₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹⁵ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₅H, —SOᵥ₁₅NH₂, —NHNH₂, —ONH₂, —NHC(O)NHNH₂, —NHC(O)NH₂, —N(O)ₘ₁₅, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹⁴ and R¹⁵ are optionally together oxo;

R¹⁶ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₆H, —SOᵥ₁₆NH₂, —NHNH₂, —ONH₂, —NHC(O)NHNH₂, —NHC(O)NH₂, —N(O)ₘ₁₆, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

R¹⁷ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₁₇H, —SOᵥ₁₇NH₂, —NHNH₂, —ONH₂, —NHC(O)NHNH₂, —NHC(O)NH₂, —N(O)ₘ₁₇, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

n12, n13, n14, n15, n16 and n17 are independently an integer from 0 to 4;

m12, m13, m14, m15, m16, m17, v12, v13, v14, v15, v16 and v17 are independently 1 or 2; and

z3 is an integer from 0 to 4.

### Embodiment P71

The compound of embodiment P70, or a pharmaceutically acceptable salt thereof, wherein z3 is 0.

### Embodiment P72

The compound of embodiment P70 or P71, or a pharmaceutically acceptable salt thereof, wherein:

X³ is

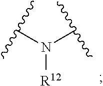

X⁴ is

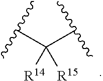

### Embodiment P73

The compound of any one of embodiments P70 or P72, or a pharmaceutically acceptable salt thereof, wherein R¹⁴ and R¹⁵ are hydrogen.

### Embodiment P74

The compound of any one of embodiments P70 or P73, or a pharmaceutically acceptable salt thereof, wherein R¹² is hydrogen.

### Embodiment P74

The compound of any one of embodiments P70 or P74, or a pharmaceutically acceptable salt thereof, wherein

X³ is

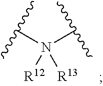

X⁴ is

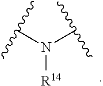

### Embodiment P76

The compound of any one of embodiments P70 to P75, or a pharmaceutically acceptable salt thereof, wherein R¹² and R¹³ are hydrogen.

### Embodiment P77

The compound of any one of embodiments P70 to P76, or a pharmaceutically acceptable salt thereof, wherein R¹⁴ is hydrogen.

### Embodiment P78

The compound of any one of embodiments P70 to P77, or a pharmaceutically acceptable salt thereof, wherein X⁵ is —O—.

### Embodiment P79

The compound of any one of embodiments P70 to P80, or a pharmaceutically acceptable salt thereof, wherein X⁵ is

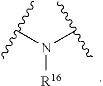

### Embodiment P80

The compound of embodiment P63 or P64, or a pharmaceutically acceptable salt thereof, wherein the compound has structural Formula (XI):

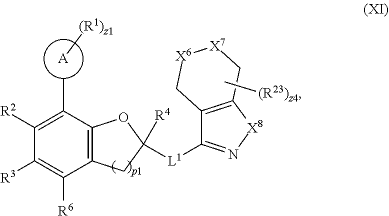

wherein:

X⁶ is

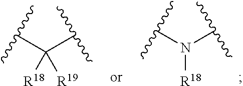

X⁷ is

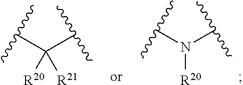

X⁸ is —O— or

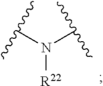

R¹⁸ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₈H, —SOᵥ₁₈NH₂, —NHNH₂, —ONH₂, —NHC(O)NHNH₂, —NHC(O)NH₂, —N(O)ₘ₁₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹⁹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₉H, —SOᵥ₁₉NH₂, —NHNH₂, —ONH₂, —NHC(O)NHNH₂, —NHC(O)NH₂, —N(O)ₘ₁₉, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹⁸ and R¹⁹ are optionally together oxo;

R²⁰ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₂₀H, —SOᵥ₂₀NH₂, —NHNH₂, —ONH₂, —NHC(O)NHNH₂, —NHC(O)NH₂, —N(O)ₘ₂₀, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²¹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₂₁H, —SOᵥ₂₁NH₂, —NHNH₂, —ONH₂, —NHC(O)NHNH₂, —NHC(O)NH₂, —N(O)ₘ₂₁, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁰ and R²¹ are optionally together oxo;

R²² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₂H, —SO₂CH₃, —SOᵥ₂₂NH₂, —NHNH₂, —ONH₂, —NHC(O)NHNH₂, —NHC(O)NH₂, —N(O)ₘ₂₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

R²³ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₂₃H, —SOᵥ₂₃NH₂, —NHNH₂, —ONH₂, —NHC(O)NHNH₂, —NHC(O)NH₂, —N(O)ₘ₂₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

n18, n19, n20, n21, n22 and n23 are independently an integer from 0 to 4;

m18, m19, m20, m21, m22, m23, v18, v19, v20, v21, v22 and v23 are independently 1 or 2; and

z4 is an integer from 0 to 4.

### Embodiment P81

The compound of embodiment P80, or a pharmaceutically acceptable salt thereof, wherein z4 is 0.

### Embodiment P82

The compound of embodiment P80 or P81 or a pharmaceutically acceptable salt thereof, wherein X⁶ is

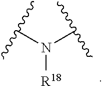

### Embodiment P83

The compound of any one of embodiments P80 to P82, or a pharmaceutically acceptable salt thereof, wherein X⁷ is

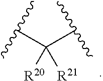

### Embodiment P84

The compound of any one of embodiments P80 to P83, or a pharmaceutically acceptable salt thereof, wherein R²⁰ is hydrogen.

### Embodiment P85

The compound of any one of embodiments P80 to P84, or a pharmaceutically acceptable salt thereof, wherein R²¹ is hydrogen.

### Embodiment P86

The compound of any one of embodiments P80 to P85, or a pharmaceutically acceptable salt thereof, wherein R¹⁸ is hydrogen.

### Embodiment P87

The compound of any one of embodiments P80 to P86, or a pharmaceutically acceptable salt thereof, wherein X⁸ is —O—.

### Embodiment P88

The compound of any one of embodiments P80 to P87, or a pharmaceutically acceptable salt thereof, wherein X⁸ is

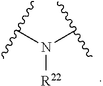

### Embodiment P89

The compound of any one of embodiments P63 to P70, or a pharmaceutically acceptable salt thereof, wherein:

R⁵ is

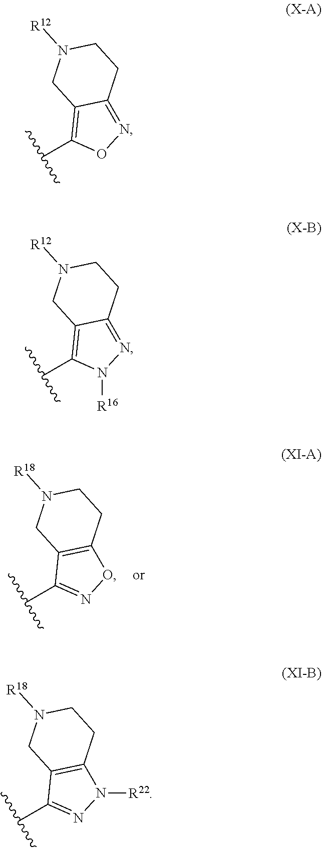

### Embodiment P90

The compound of any one of embodiments P63, P64, or P71, or a pharmaceutically acceptable salt thereof, wherein the compound has structural Formula

(X-1A):

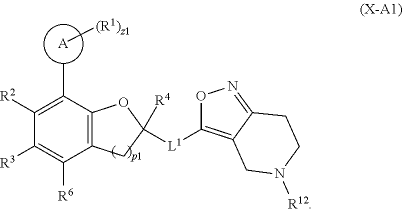

### Embodiment P91

The compound of embodiment P63 of P64, or a pharmaceutically acceptable salt thereof, wherein the compound has structural Formula (XII):

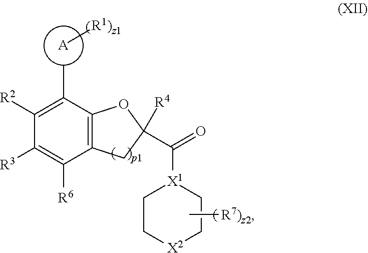

wherein:

X¹ is or

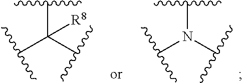

X² is

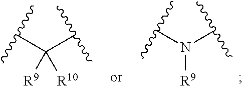

n7, n8, n9 and n10 are independently an integer from 0 to 4;

m7, m8, m9, m10, v7, v8, v9 and v10 are independently 1 or 2;

z2 is an integer from 0 to 8;

R⁷ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₇H, —SOᵥ₇NH₂, —NHNH₂, —ONH₂, —NHC(O)NHNH₂, —NHC(O)NH₂, —N(O)ₘ₇, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁸ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₈H, —SOᵥ₈NH₂, —NHNH₂, —ONH₂, —NHC(O)NHNH₂, —NHC(O)NH₂, —N(O)ₘ₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₉R^{9A}, —SOᵥ₉NR^{9B}R^{9C}, —NHNR^{9B}R^{9C}, —ONR^{9B}R^{9C}, —NHC(O)NHNR^{9B}R^{9C}, —NHC(O)NR^{9B}R^{9C}, —N(O)ₘ₉, —NR^{9B}R^{9C}, —C(O)R^{9D}, —C(O)OR^{9D}, —C(O)NR^{9B}R^{9C}, —OR^{9A}, —NR^{9B}SO₂R^{9A}, —NR^{9B}C(O)R^{9D}, —NR^{9B}C(O)OR^{9D}, —NR^{9B}OR^{9D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹⁰ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₁₀R^{10A}, —SOᵥ₁₀NR^{10B}R^{10C}, —NHNR^{10B}R^{10C}, —ONR^{10B}R^{1C}, —NHC(O)NHNR^{10B}R^{10C}, —NHC(O)NR^{10B}R^{10C}, —N(O)ₘ₁₀, —NR^{10B}R^{10C}, —C(O)R^{10D}, —C(O)OR^{10D}, —C(O)NR^{10B}R^{10C}, —OR^{10A}, —NR^{10B}SO₂R^{10A}, —NR^{10B}C(O)R^{10D}, —NR^{10B}C(O)OR^{10D}, —NR^{10B}OR^{10D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁹ and R¹⁰ are optionally together oxo; and

R^{9A}, R^{9B}, R^{9C}, R^{9D}, R^{10A}, R^{10B}, R^{10C} and R^{10D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{9B} and R^{9C} or R^{10B} and R^{10C} substituents bonded to the same nitrogen atom may optionally be joined to form a substituted or unsubstituted heterocycloalkyl or substituted or unsubstituted heteroaryl.

### Embodiment P92

The compound of embodiment P91, or a pharmaceutically acceptable salt thereof, wherein X¹ is

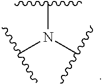 [SMILES: *N(*)*]

### Embodiment P93

The compound of embodiment P91, or a pharmaceutically acceptable salt thereof, wherein X¹ is

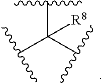

### Embodiment P94

The compound of any one of embodiments P91 to P93, or a pharmaceutically acceptable salt thereof, wherein X² is

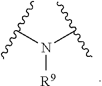

### Embodiment P95

The compound of any one of embodiments P91 to P93, or a pharmaceutically acceptable salt thereof, wherein X² is

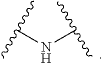 [SMILES: *N*]

### Embodiment P96

The compound of any one of embodiments P91 to P93, or a pharmaceutically acceptable salt thereof, wherein X² is

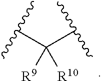

### Embodiment P97

The compound of any one of embodiments P91 to P96, or a pharmaceutically acceptable salt thereof, wherein R⁹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment P98

The compound of any one of embodiments P91 to P96, or a pharmaceutically acceptable salt thereof, wherein R⁹ is substituted or unsubstituted cycloalkyl, substituted or unsubstituted heterocycloalkyl, substituted or unsubstituted aryl, or substituted or unsubstituted heteroaryl.

### Embodiment P99

The compound of any one of embodiments P91 to P96, or a pharmaceutically acceptable salt thereof, wherein R⁹ is substituted or unsubstituted pyridinyl.

### Embodiment P100

The compound of any one of embodiments P85 to P99, or a pharmaceutically acceptable salt thereof, wherein Ring B is substituted or unsubstituted heterocycloalkyl, substituted or unsubstituted aryl, or substituted or unsubstituted heteroaryl.

### Embodiment P101

The compound of any one of embodiments P85 to P99, or a pharmaceutically acceptable salt thereof, wherein Ring B is a substituted or unsubstituted monovalent succinimide moiety, substituted or unsubstituted monovalent hydantoin moiety, substituted or unsubstituted monovalent oxohydantoin moiety, substituted or unsubstituted monovalent thiohydantoin moiety, substituted or unsubstituted monovalent pyrrolidone moiety, substituted or unsubstituted monovalent phthalimide moiety, substituted or unsubstituted monovalent oxazolidinone moiety, or substituted or unsubstituted monovalent cyclic urea moiety.

### Embodiment P102

The compound of any one of embodiments P85 to P99, or a pharmaceutically acceptable salt thereof, wherein:

Ring B—(R⁵⁹)z9 is

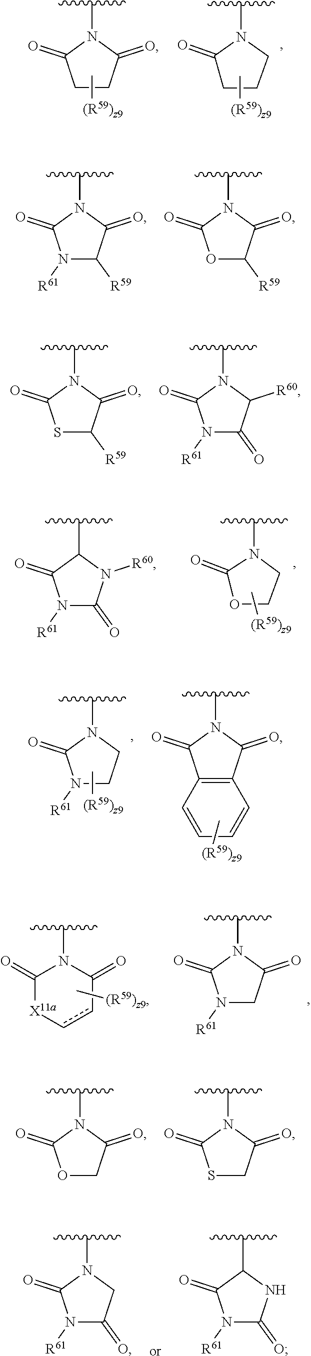

wherein:

X¹¹ is

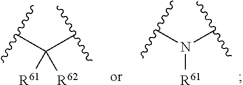

[symbol] is a single bond or double bond;

R⁶¹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₆₁R^{61A}, —SO^{61B}R^{61C}, —NHNR^{61B}R^{61C}, —ONR^{61B}R^{61C}, —NHC(O)NHNR^{61B}R^{61C}, —NHC(O)NR^{61B}R^{61C}, —N(O)ₘ₆₁, —NR^{61B}R^{61C}, —C(O)R^{61D}, —C(O)OR^{61D}, —C(O)NR^{61B}R^{61C}, —OR^{61A}, —NR^{61B}SO₂R^{61A}, —N^{61B}C(O)R^{61D}, —NR^{61B}C(O)OR^{61D}, —NR^{61B}OR^{61D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁶² is independently hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₆₂H, —SOᵥ₆₂NH₂, —NHNH₂, —ONH₂, —NHC(O)NHNH₂, —NHC(O)NH₂, —N(O)ₘ₆₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁶¹ and R⁶² are optionally together oxo; and

R^{61A}, R^{61B}, R^{61C} and R^{61D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{61B} and R^{61C} substituents bonded to the same nitrogen atom may optionally be joined to form a substituted or unsubstituted heterocycloalkyl or substituted or unsubstituted heteroaryl;

n61 and n62 are independently an integer from 0 to 4; and

m61, m62, v61 and v62 are independently 1 or 2.

### Embodiment P103

The compound of any one of embodiments P64 to P102, or a pharmaceutically acceptable salt thereof, wherein R²⁴ and R²⁵ are independently —CN, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment P104

The compound of any one of embodiments P64 to P102, or a pharmaceutically acceptable salt thereof, wherein R²⁴ and R²⁵ are independently —CH₂OH, —C(O)NH₂, —C(O)NHCH₃, —CH(OH)CH₂CH₃ or —CH(OH)CH₃.

### Embodiment P105

The compound of any one of embodiments P63 to P104, or a pharmaceutically acceptable salt thereof, wherein R¹ is halogen, —CN, substituted or unsubstituted alkyl or substituted or unsubstituted heteroalkyl.

### Embodiment P106

The compound of any one of embodiments P63 to P105, or a pharmaceutically acceptable salt thereof, wherein R⁴ is hydrogen, substituted or unsubstituted alkyl or substituted or unsubstituted heteroalkyl.

### Embodiment P107

The compound of any one of embodiments P63 to P106, or a pharmaceutically acceptable salt thereof, wherein R⁴ is hydrogen or unsubstituted methyl.

### Embodiment P108

The compound of any one of embodiments P63 to P107, or a pharmaceutically acceptable salt thereof, wherein R² and R³ are independently hydrogen, halogen, substituted or unsubstituted alkyl or substituted or unsubstituted heteroalkyl.

### Embodiment P109

The compound of any one of embodiments P63 to P108, or a pharmaceutically acceptable salt thereof, wherein R² and R³ are unsubstituted methyl.

### Embodiment P110

The compound of any one of embodiments P63 to P108, or a pharmaceutically acceptable salt thereof, wherein R² is hydrogen.

### Embodiment P111

The compound of any one of embodiments P63 to P108, or a pharmaceutically acceptable salt thereof, wherein R³ is halogen, unsubstituted methyl or —CF₃.

### Embodiment P112

The compound of any one of embodiments P71 to P92, or a pharmaceutically acceptable salt thereof, wherein R¹² is —N(CH₃)₂ or —NHCH₃.

### Embodiment P113

The compound of any one of embodiments P71 to P92, or a pharmaceutically acceptable salt thereof, wherein R¹² is —N(CH₃)₂.

### Embodiment P114

The compound of any one of embodiments P63 to P113, or a pharmaceutically acceptable salt thereof, wherein X⁹ is —S— or —O—.

### Embodiment P115

The compound of of any one of embodiments P63 to P114, or a pharmaceutically acceptable salt thereof, wherein p1 is 1.

### Embodiment P116

The compound of embodiment P63, wherein the compound is:

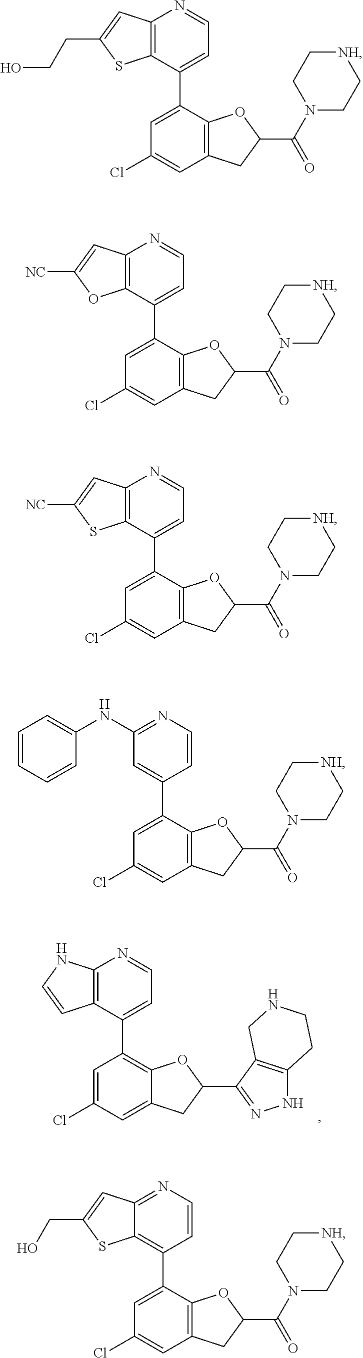 [SMILES: O=C(C1Cc2cc(Cl)cc(-c3ccnc4cc(CCO)sc34)c2O1)N1CCNCC1 ; N#Cc1cc2nccc(-c3cc(Cl)cc4c3OC(C(=O)N3CCNCC3)C4)c2o1 ; N#Cc1cc2nccc(-c3cc(Cl)cc4c3OC(C(=O)N3CCNCC3)C4)c2s1 ; O=C(C1Cc2cc(Cl)cc(-c3ccnc(Nc4ccccc4)c3)c2O1)N1CCNCC1 ; Clc1cc2c(c(-c3ccnc4[nH]ccc34)c1)OC(c1n[nH]c3c1CNCC3)C2 ; O=C(C1Cc2cc(Cl)cc(-c3ccnc4cc(CO)sc34)c2O1)N1CCNCC1]

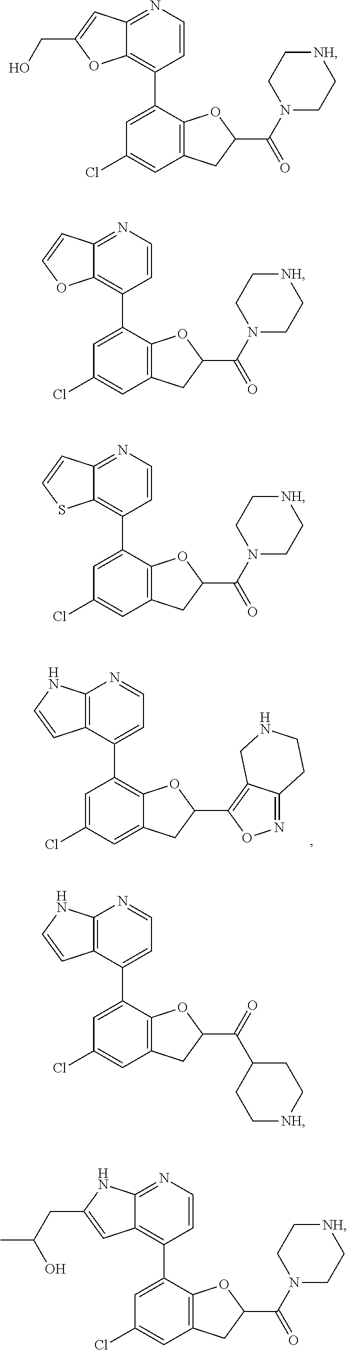 [SMILES: O=C(C1Cc2cc(Cl)cc(-c3ccnc4cc(CO)oc34)c2O1)N1CCNCC1 ; O=C(C1Cc2cc(Cl)cc(-c3ccnc4ccoc34)c2O1)N1CCNCC1 ; O=C(C1Cc2cc(Cl)cc(-c3ccnc4ccsc34)c2O1)N1CCNCC1 ; Clc1cc2c(c(-c3ccnc4[nH]ccc34)c1)OC(c1onc3c1CNCC3)C2 ; O=C(C1CCNCC1)C1Cc2cc(Cl)cc(-c3ccnc4[nH]ccc34)c2O1 ; CC(O)Cc1cc2c(-c3cc(Cl)cc4c3OC(C(=O)N3CCNCC3)C4)ccnc2[nH]1]

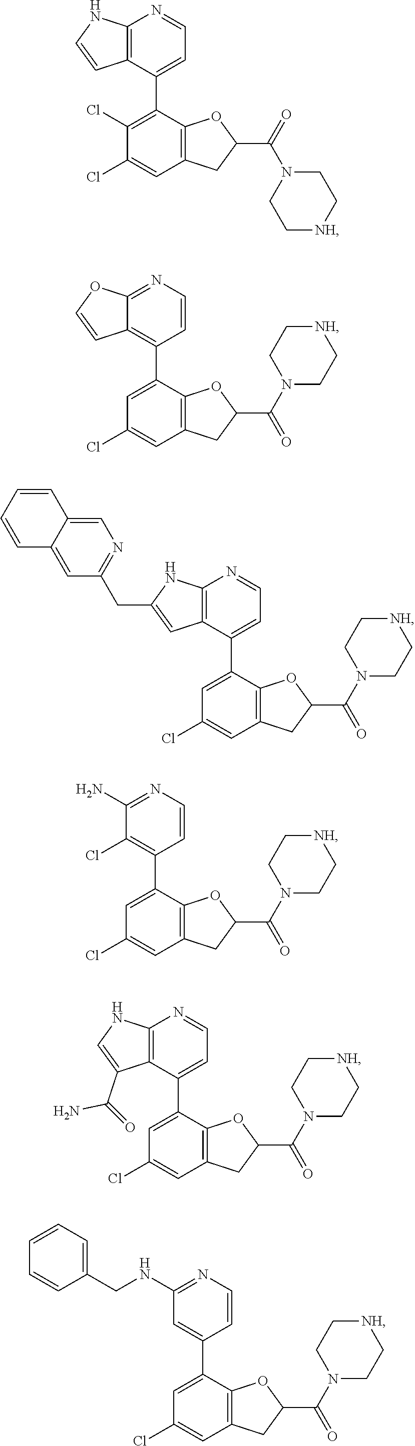 [SMILES: O=C(C1Cc2cc(Cl)c(Cl)c(-c3ccnc4[nH]ccc34)c2O1)N1CCNCC1 ; O=C(C1Cc2cc(Cl)cc(-c3ccnc4occc34)c2O1)N1CCNCC1 ; O=C(C1Cc2cc(Cl)cc(-c3ccnc4[nH]c(Cc5cc6ccccc6cn5)cc34)c2O1)N1CCNCC1 ; Nc1nccc(-c2cc(Cl)cc3c2OC(C(=O)N2CCNCC2)C3)c1Cl ; NC(=O)c1c[nH]c2nccc(-c3cc(Cl)cc4c3OC(C(=O)N3CCNCC3)C4)c12 ; O=C(C1Cc2cc(Cl)cc(-c3ccnc(NCc4ccccc4)c3)c2O1)N1CCNCC1]

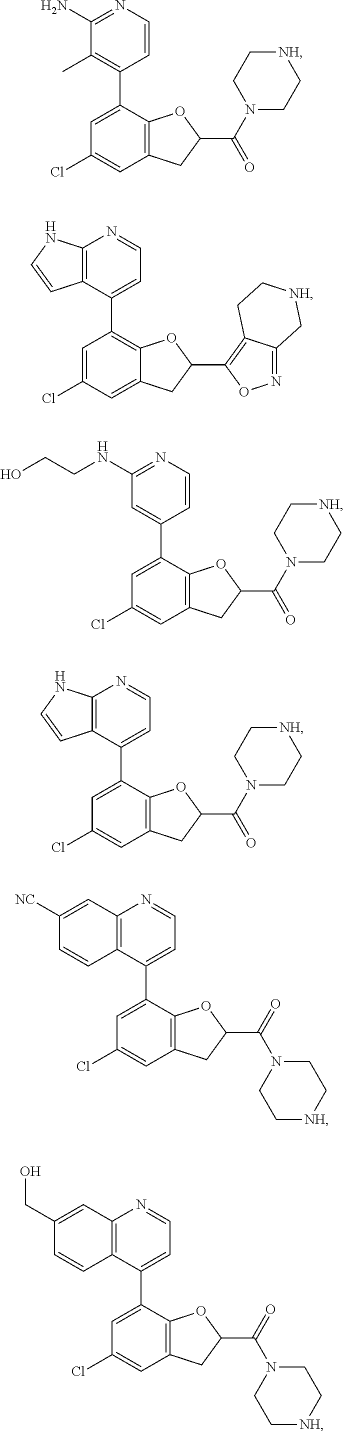 [SMILES: Cc1c(-c2cc(Cl)cc3c2OC(C(=O)N2CCNCC2)C3)ccnc1N ; Clc1cc2c(c(-c3ccnc4[nH]ccc34)c1)OC(c1onc3c1CCNC3)C2 ; O=C(C1Cc2cc(Cl)cc(-c3ccnc(NCCO)c3)c2O1)N1CCNCC1 ; O=C(C1Cc2cc(Cl)cc(-c3ccnc4[nH]ccc34)c2O1)N1CCNCC1 ; N#Cc1ccc2c(-c3cc(Cl)cc4c3OC(C(=O)N3CCNCC3)C4)ccnc2c1 ; O=C(C1Cc2cc(Cl)cc(-c3ccnc4cc(CO)ccc34)c2O1)N1CCNCC1]

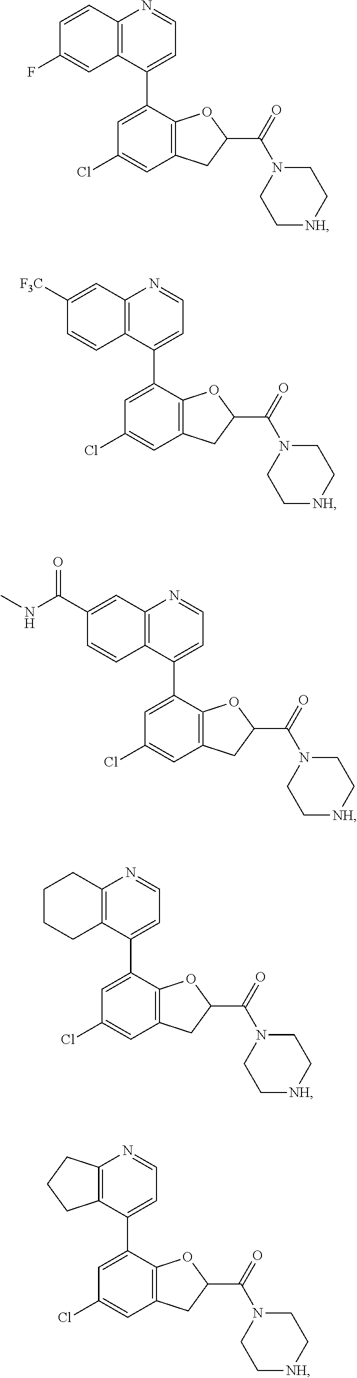 [SMILES: O=C(C1Cc2cc(Cl)cc(-c3ccnc4ccc(F)cc34)c2O1)N1CCNCC1 ; O=C(C1Cc2cc(Cl)cc(-c3ccnc4cc(C(F)(F)F)ccc34)c2O1)N1CCNCC1 ; CNC(=O)c1ccc2c(-c3cc(Cl)cc4c3OC(C(=O)N3CCNCC3)C4)ccnc2c1 ; O=C(C1Cc2cc(Cl)cc(-c3ccnc4c3CCCC4)c2O1)N1CCNCC1 ; O=C(C1Cc2cc(Cl)cc(-c3ccnc4c3CCC4)c2O1)N1CCNCC1]

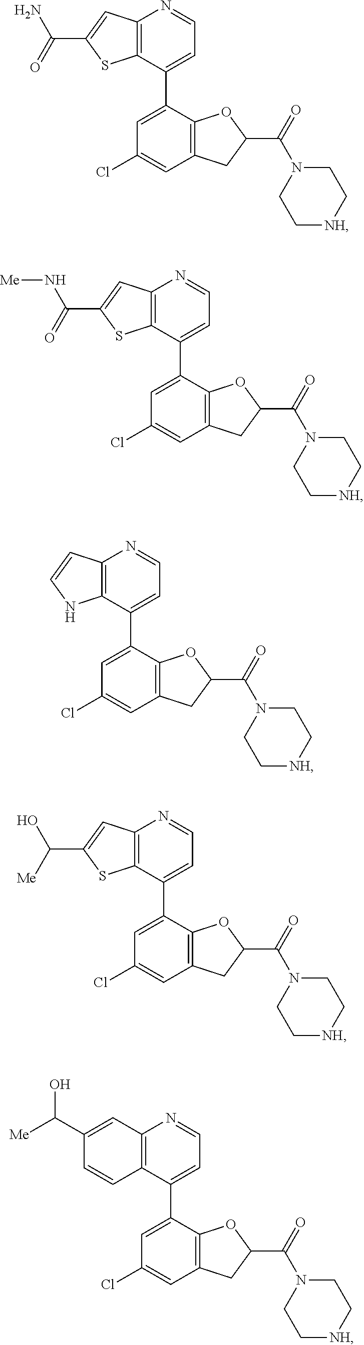 [SMILES: NC(=O)c1cc2nccc(-c3cc(Cl)cc4c3OC(C(=O)N3CCNCC3)C4)c2s1 ; CNC(=O)c1cc2nccc(-c3cc(Cl)cc4c3OC(C(=O)N3CCNCC3)C4)c2s1 ; O=C(C1Cc2cc(Cl)cc(-c3ccnc4cc[nH]c34)c2O1)N1CCNCC1 ; CC(O)c1cc2nccc(-c3cc(Cl)cc4c3OC(C(=O)N3CCNCC3)C4)c2s1 ; CC(O)c1ccc2c(-c3cc(Cl)cc4c3OC(C(=O)N3CCNCC3)C4)ccnc2c1]

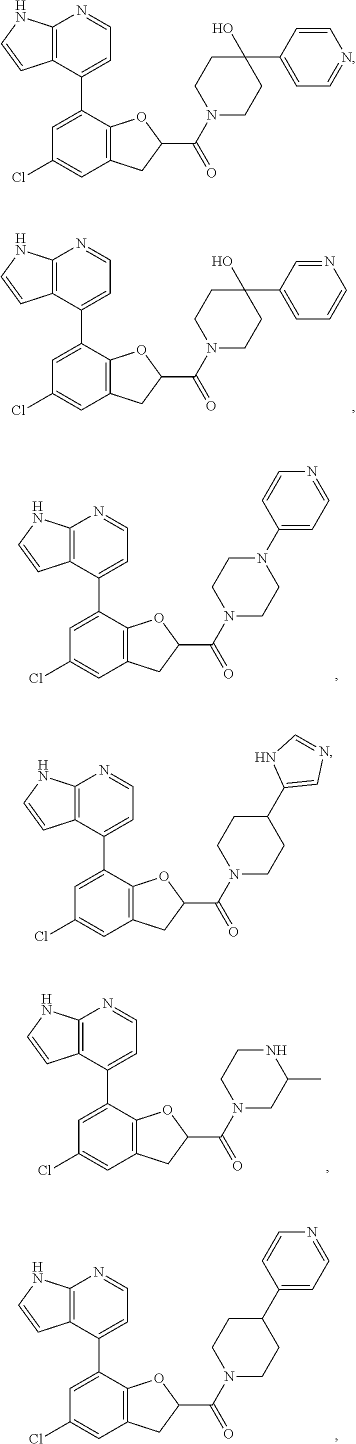 [SMILES: O=C(C1Cc2cc(Cl)cc(-c3ccnc4[nH]ccc34)c2O1)N1CCC(O)(c2ccncc2)CC1 ; O=C(C1Cc2cc(Cl)cc(-c3ccnc4[nH]ccc34)c2O1)N1CCC(O)(c2cccnc2)CC1 ; O=C(C1Cc2cc(Cl)cc(-c3ccnc4[nH]ccc34)c2O1)N1CCN(c2ccncc2)CC1 ; O=C(C1Cc2cc(Cl)cc(-c3ccnc4[nH]ccc34)c2O1)N1CCC(c2cnc[nH]2)CC1 ; CC1CN(C(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(c2ccncc2)CC1]

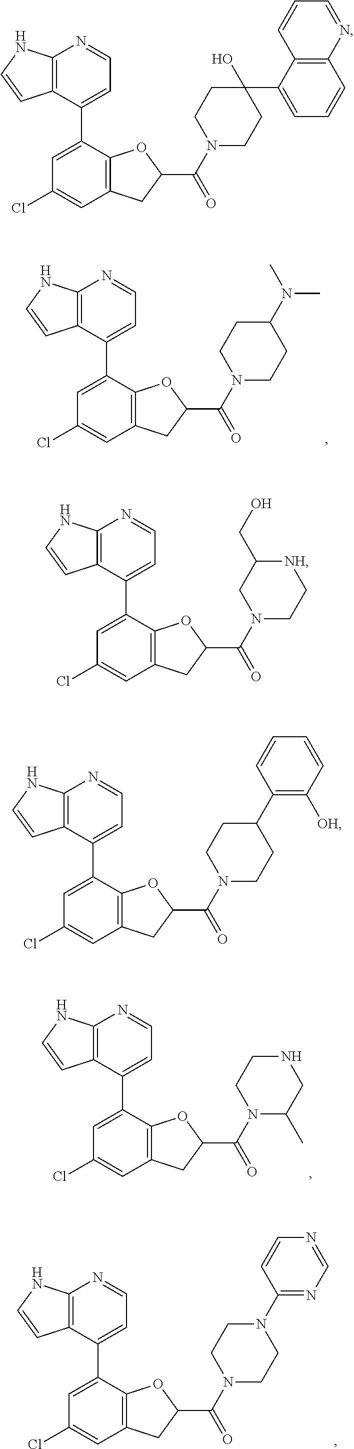 [SMILES: O=C(C1Cc2cc(Cl)cc(-c3ccnc4[nH]ccc34)c2O1)N1CCC(O)(c2cccc3ncccc23)CC1 ; CN(C)C1CCN(C(=O)C2Cc3cc(Cl)cc(-c4ccnc5[nH]ccc45)c3O2)CC1 ; O=C(C1Cc2cc(Cl)cc(-c3ccnc4[nH]ccc34)c2O1)N1CCNC(CO)C1 ; O=C(C1Cc2cc(Cl)cc(-c3ccnc4[nH]ccc34)c2O1)N1CCC(c2ccccc2O)CC1 ; CC1CNCCN1C(=O)C1Cc2cc(Cl)cc(-c3ccnc4[nH]ccc34)c2O1 ; O=C(C1Cc2cc(Cl)cc(-c3ccnc4[nH]ccc34)c2O1)N1CCN(c2ccncn2)CC1]

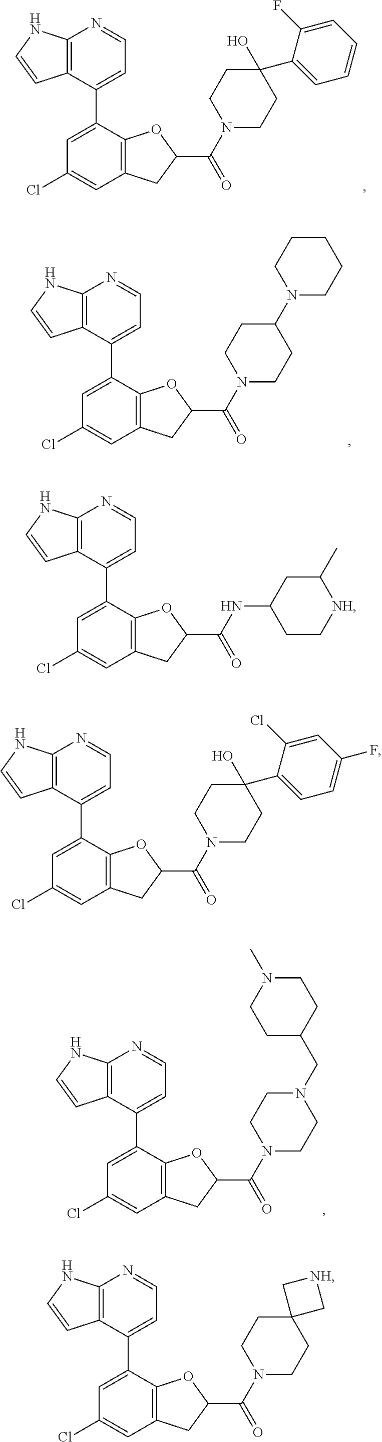 [SMILES: O=C(C1Cc2cc(Cl)cc(-c3ccnc4[nH]ccc34)c2O1)N1CCC(O)(c2ccccc2F)CC1 ; O=C(C1Cc2cc(Cl)cc(-c3ccnc4[nH]ccc34)c2O1)N1CCC(N2CCCCC2)CC1 ; CC1CC(NC(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(O)(c2ccc(F)cc2Cl)CC1 ; CN1CCC(CN2CCN(C(=O)C3Cc4cc(Cl)cc(-c5ccnc6[nH]ccc56)c4O3)CC2)CC1 ; O=C(C1Cc2cc(Cl)cc(-c3ccnc4[nH]ccc34)c2O1)N1CCC2(CC1)CNC2]

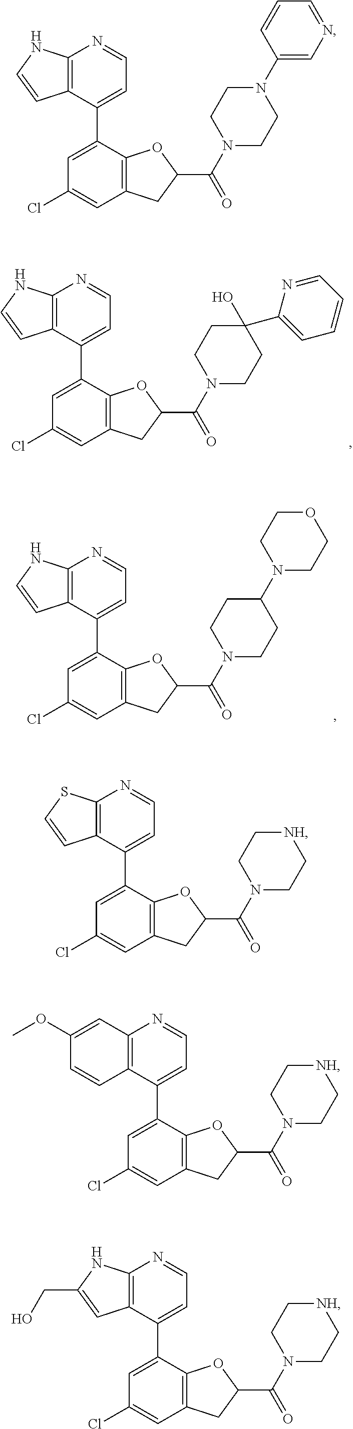 [SMILES: O=C(C1Cc2cc(Cl)cc(-c3ccnc4[nH]ccc34)c2O1)N1CCN(c2cccnc2)CC1 ; O=C(C1Cc2cc(Cl)cc(-c3ccnc4[nH]ccc34)c2O1)N1CCC(O)(c2ccccn2)CC1 ; O=C(C1Cc2cc(Cl)cc(-c3ccnc4[nH]ccc34)c2O1)N1CCC(N2CCOCC2)CC1 ; O=C(C1Cc2cc(Cl)cc(-c3ccnc4sccc34)c2O1)N1CCNCC1 ; COc1ccc2c(-c3cc(Cl)cc4c3OC(C(=O)N3CCNCC3)C4)ccnc2c1 ; O=C(C1Cc2cc(Cl)cc(-c3ccnc4[nH]c(CO)cc34)c2O1)N1CCNCC1]

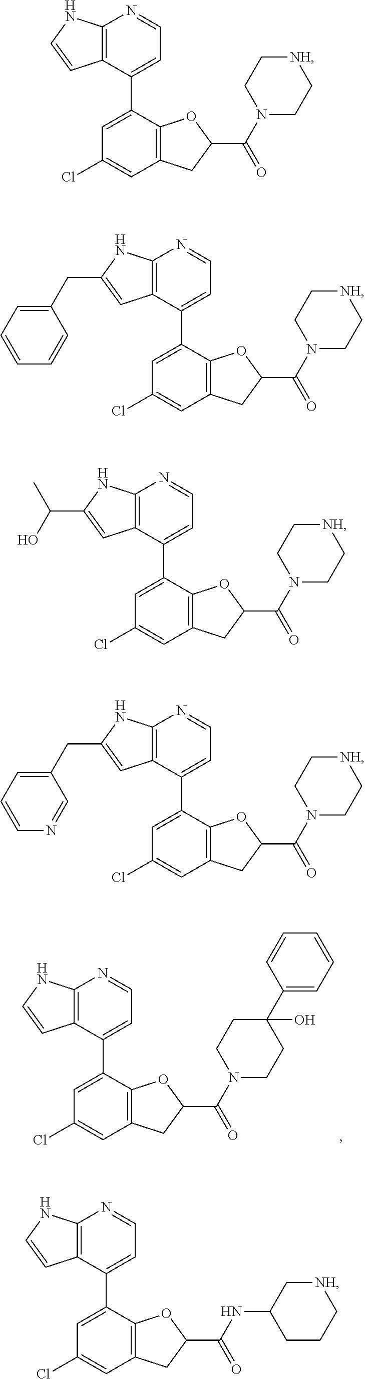 [SMILES: O=C(C1Cc2cc(Cl)cc(-c3ccnc4[nH]ccc34)c2O1)N1CCNCC1 ; O=C(C1Cc2cc(Cl)cc(-c3ccnc4[nH]c(Cc5ccccc5)cc34)c2O1)N1CCNCC1 ; CC(O)c1cc2c(-c3cc(Cl)cc4c3OC(C(=O)N3CCNCC3)C4)ccnc2[nH]1 ; O=C(C1Cc2cc(Cl)cc(-c3ccnc4[nH]c(Cc5cccnc5)cc34)c2O1)N1CCNCC1 ; O=C(C1Cc2cc(Cl)cc(-c3ccnc4[nH]ccc34)c2O1)N1CCC(O)(c2ccccc2)CC1 ; O=C(NC1CCCNC1)C1Cc2cc(Cl)cc(-c3ccnc4[nH]ccc34)c2O1]

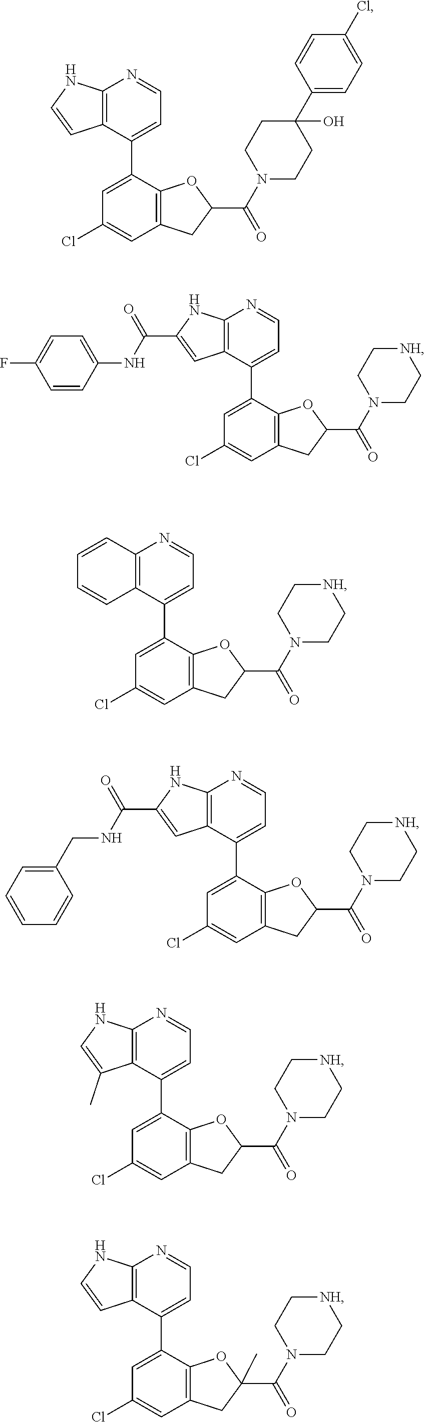 [SMILES: O=C(C1Cc2cc(Cl)cc(-c3ccnc4[nH]ccc34)c2O1)N1CCC(O)(c2ccc(Cl)cc2)CC1 ; O=C(Nc1ccc(F)cc1)c1cc2c(-c3cc(Cl)cc4c3OC(C(=O)N3CCNCC3)C4)ccnc2[nH]1 ; O=C(C1Cc2cc(Cl)cc(-c3ccnc4ccccc34)c2O1)N1CCNCC1 ; O=C(NCc1ccccc1)c1cc2c(-c3cc(Cl)cc4c3OC(C(=O)N3CCNCC3)C4)ccnc2[nH]1 ; Cc1c[nH]c2nccc(-c3cc(Cl)cc4c3OC(C(=O)N3CCNCC3)C4)c12 ; CC1(C(=O)N2CCNCC2)Cc2cc(Cl)cc(-c3ccnc4[nH]ccc34)c2O1]

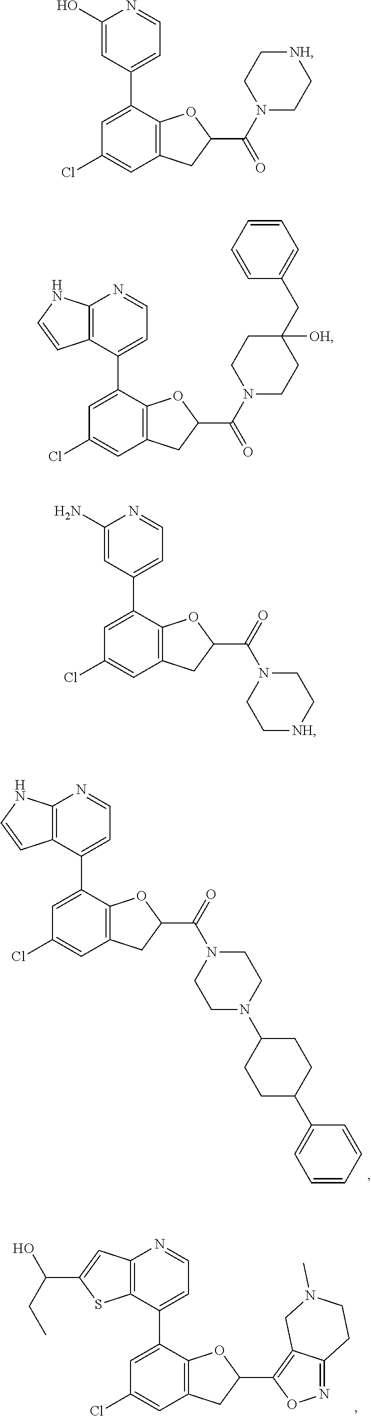 [SMILES: O=C(C1Cc2cc(Cl)cc(-c3ccnc(O)c3)c2O1)N1CCNCC1 ; O=C(C1Cc2cc(Cl)cc(-c3ccnc4[nH]ccc34)c2O1)N1CCC(O)(Cc2ccccc2)CC1 ; Nc1cc(-c2cc(Cl)cc3c2OC(C(=O)N2CCNCC2)C3)ccn1 ; O=C(C1Cc2cc(Cl)cc(-c3ccnc4[nH]ccc34)c2O1)N1CCN(C2CCC(c3ccccc3)CC2)CC1 ; CCC(O)c1cc2nccc(-c3cc(Cl)cc4c3OC(c3onc5c3CN(C)CC5)C4)c2s1]

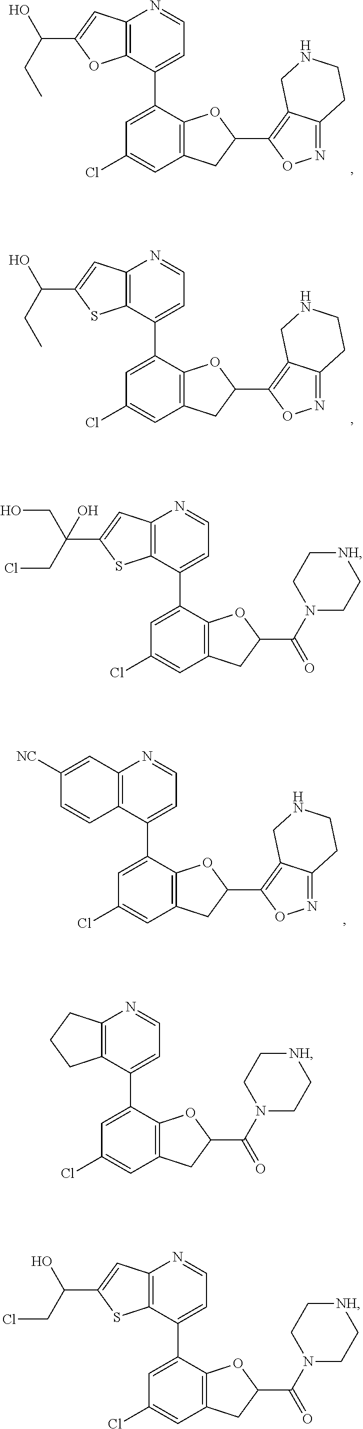 [SMILES: CCC(O)c1cc2nccc(-c3cc(Cl)cc4c3OC(c3onc5c3CNCC5)C4)c2o1 ; CCC(O)c1cc2nccc(-c3cc(Cl)cc4c3OC(c3onc5c3CNCC5)C4)c2s1 ; O=C(C1Cc2cc(Cl)cc(-c3ccnc4cc(C(O)(CO)CCl)sc34)c2O1)N1CCNCC1 ; N#Cc1ccc2c(-c3cc(Cl)cc4c3OC(c3onc5c3CNCC5)C4)ccnc2c1 ; O=C(C1Cc2cc(Cl)cc(-c3ccnc4c3CCC4)c2O1)N1CCNCC1 ; O=C(C1Cc2cc(Cl)cc(-c3ccnc4cc(C(O)CCl)sc34)c2O1)N1CCNCC1]

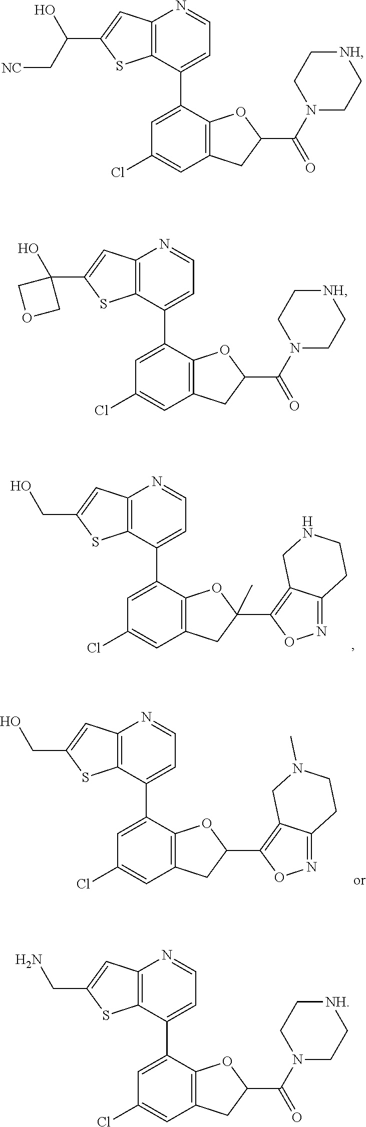 [SMILES: N#CCC(O)c1cc2nccc(-c3cc(Cl)cc4c3OC(C(=O)N3CCNCC3)C4)c2s1 ; O=C(C1Cc2cc(Cl)cc(-c3ccnc4cc(C5(O)COC5)sc34)c2O1)N1CCNCC1 ; CC1(c2onc3c2CNCC3)Cc2cc(Cl)cc(-c3ccnc4cc(CO)sc34)c2O1 ; CN1CCc2noc(C3Cc4cc(Cl)cc(-c5ccnc6cc(CO)sc56)c4O3)c2C1 ; NCc1cc2nccc(-c3cc(Cl)cc4c3OC(C(=O)N3CCNCC3)C4)c2s1]

### Embodiment P117

The compound of embodiment P63, wherein the compound is:

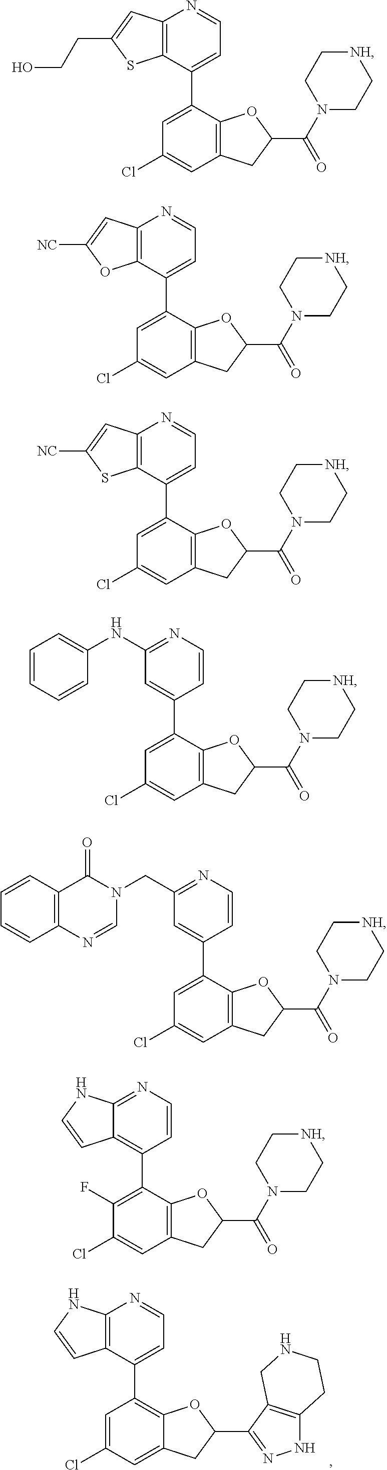

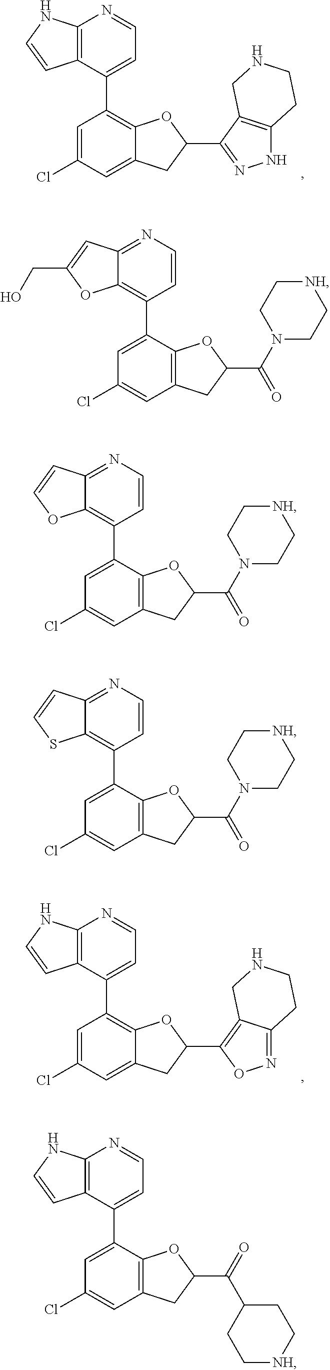 [SMILES: Clc1cc2c(c(-c3ccnc4[nH]ccc34)c1)OC(c1n[nH]c3c1CNCC3)C2 ; O=C(C1Cc2cc(Cl)cc(-c3ccnc4cc(CO)oc34)c2O1)N1CCNCC1 ; O=C(C1Cc2cc(Cl)cc(-c3ccnc4ccoc34)c2O1)N1CCNCC1 ; O=C(C1Cc2cc(Cl)cc(-c3ccnc4ccsc34)c2O1)N1CCNCC1 ; Clc1cc2c(c(-c3ccnc4[nH]ccc34)c1)OC(c1onc3c1CNCC3)C2 ; O=C(C1CCNCC1)C1Cc2cc(Cl)cc(-c3ccnc4[nH]ccc34)c2O1]

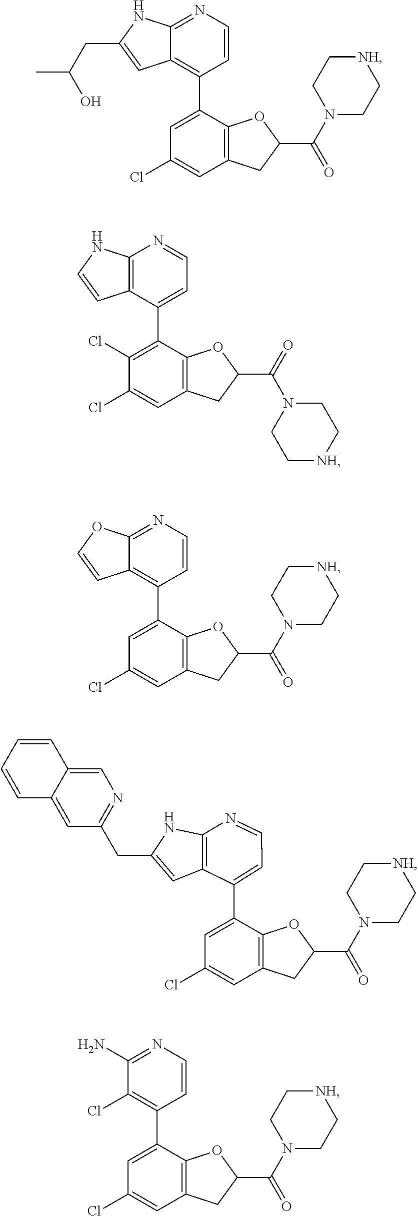 [SMILES: CC(O)Cc1cc2c(-c3cc(Cl)cc4c3OC(C(=O)N3CCNCC3)C4)ccnc2[nH]1 ; O=C(C1Cc2cc(Cl)c(Cl)c(-c3ccnc4[nH]ccc34)c2O1)N1CCNCC1 ; O=C(C1Cc2cc(Cl)cc(-c3ccnc4occc34)c2O1)N1CCNCC1 ; O=C(C1Cc2cc(Cl)cc(-c3ccnc4[nH]c(Cc5cc6ccccc6cn5)cc34)c2O1)N1CCNCC1 ; Nc1nccc(-c2cc(Cl)cc3c2OC(C(=O)N2CCNCC2)C3)c1Cl]

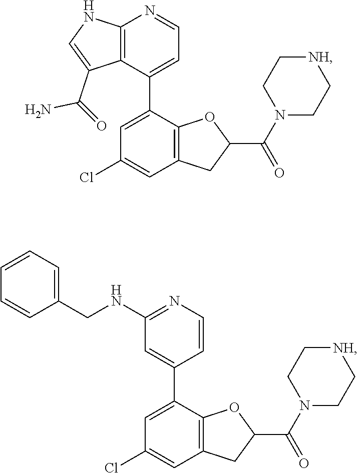 [SMILES: NC(=O)c1c[nH]c2nccc(-c3cc(Cl)cc4c3OC(C(=O)N3CCNCC3)C4)c12 ; O=C(C1Cc2cc(Cl)cc(-c3ccnc(NCc4ccccc4)c3)c2O1)N1CCNCC1]

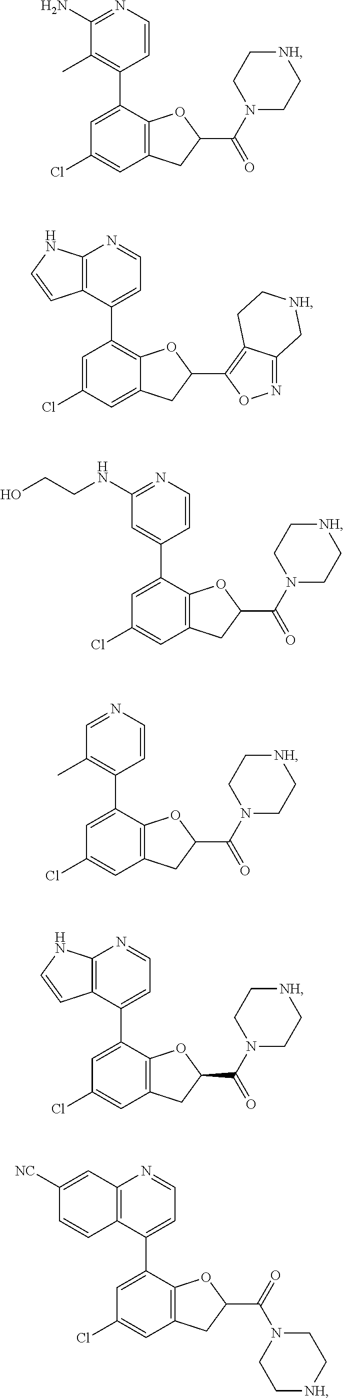 [SMILES: Cc1c(-c2cc(Cl)cc3c2OC(C(=O)N2CCNCC2)C3)ccnc1N ; Clc1cc2c(c(-c3ccnc4[nH]ccc34)c1)OC(c1onc3c1CCNC3)C2 ; O=C(C1Cc2cc(Cl)cc(-c3ccnc(NCCO)c3)c2O1)N1CCNCC1 ; Cc1cnccc1-c1cc(Cl)cc2c1OC(C(=O)N1CCNCC1)C2 ; O=C([C@H]1Cc2cc(Cl)cc(-c3ccnc4[nH]ccc34)c2O1)N1CCNCC1 ; N#Cc1ccc2c(-c3cc(Cl)cc4c3OC(C(=O)N3CCNCC3)C4)ccnc2c1]

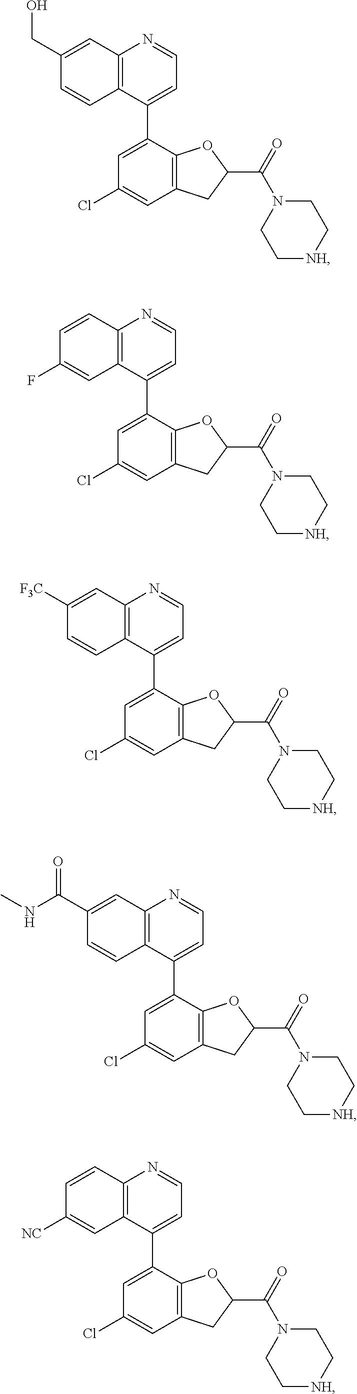 [SMILES: O=C(C1Cc2cc(Cl)cc(-c3ccnc4cc(CO)ccc34)c2O1)N1CCNCC1 ; O=C(C1Cc2cc(Cl)cc(-c3ccnc4ccc(F)cc34)c2O1)N1CCNCC1 ; O=C(C1Cc2cc(Cl)cc(-c3ccnc4cc(C(F)(F)F)ccc34)c2O1)N1CCNCC1 ; CNC(=O)c1ccc2c(-c3cc(Cl)cc4c3OC(C(=O)N3CCNCC3)C4)ccnc2c1 ; N#Cc1ccc2nccc(-c3cc(Cl)cc4c3OC(C(=O)N3CCNCC3)C4)c2c1]

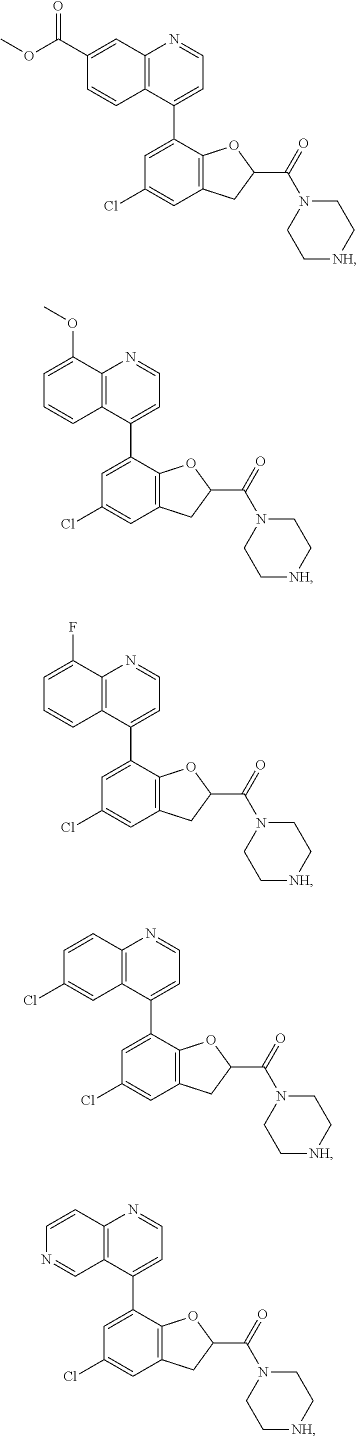 [SMILES: COC(=O)c1ccc2c(-c3cc(Cl)cc4c3OC(C(=O)N3CCNCC3)C4)ccnc2c1 ; COc1cccc2c(-c3cc(Cl)cc4c3OC(C(=O)N3CCNCC3)C4)ccnc12 ; O=C(C1Cc2cc(Cl)cc(-c3ccnc4c(F)cccc34)c2O1)N1CCNCC1 ; O=C(C1Cc2cc(Cl)cc(-c3ccnc4ccc(Cl)cc34)c2O1)N1CCNCC1 ; O=C(C1Cc2cc(Cl)cc(-c3ccnc4ccncc34)c2O1)N1CCNCC1]

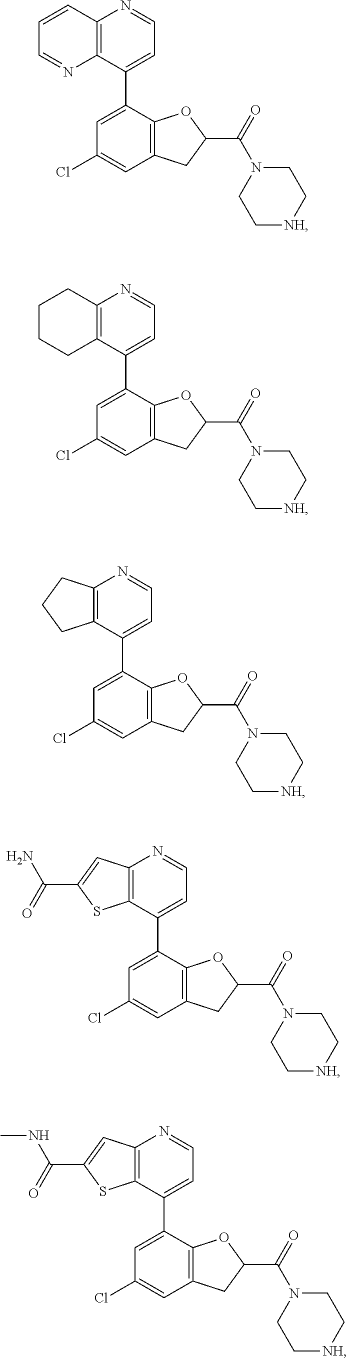 [SMILES: O=C(C1Cc2cc(Cl)cc(-c3ccnc4cccnc34)c2O1)N1CCNCC1 ; O=C(C1Cc2cc(Cl)cc(-c3ccnc4c3CCCC4)c2O1)N1CCNCC1 ; O=C(C1Cc2cc(Cl)cc(-c3ccnc4c3CCC4)c2O1)N1CCNCC1 ; NC(=O)c1cc2nccc(-c3cc(Cl)cc4c3OC(C(=O)N3CCNCC3)C4)c2s1 ; CNC(=O)c1cc2nccc(-c3cc(Cl)cc4c3OC(C(=O)N3CCNCC3)C4)c2s1]

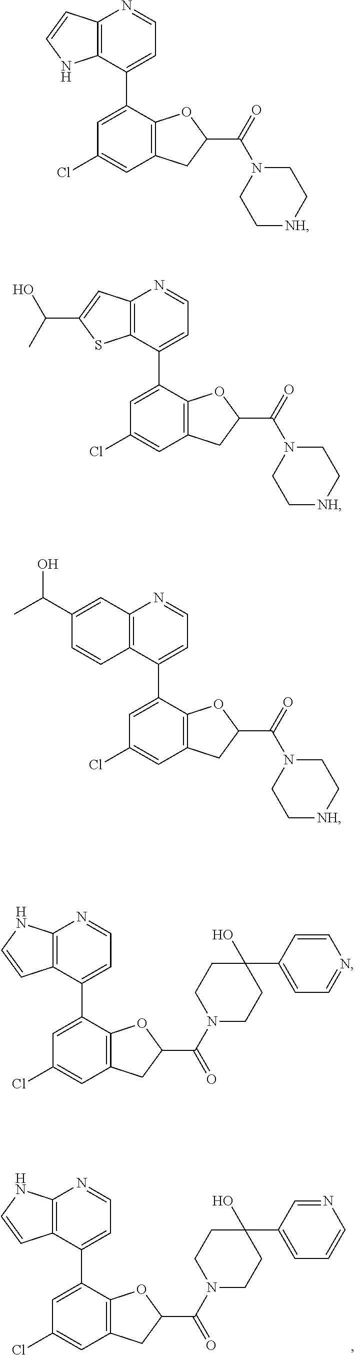 [SMILES: O=C(C1Cc2cc(Cl)cc(-c3ccnc4cc[nH]c34)c2O1)N1CCNCC1 ; CC(O)c1cc2nccc(-c3cc(Cl)cc4c3OC(C(=O)N3CCNCC3)C4)c2s1 ; CC(O)c1ccc2c(-c3cc(Cl)cc4c3OC(C(=O)N3CCNCC3)C4)ccnc2c1 ; O=C(C1Cc2cc(Cl)cc(-c3ccnc4[nH]ccc34)c2O1)N1CCC(O)(c2ccncc2)CC1 ; O=C(C1Cc2cc(Cl)cc(-c3ccnc4[nH]ccc34)c2O1)N1CCC(O)(c2cccnc2)CC1]

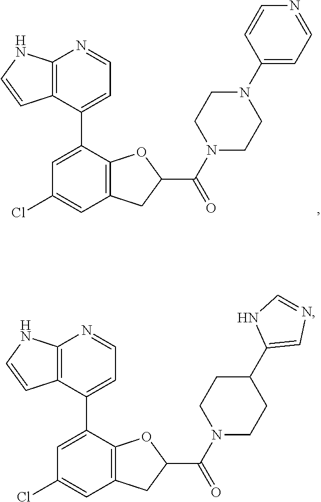 [SMILES: O=C(C1Cc2cc(Cl)cc(-c3ccnc4[nH]ccc34)c2O1)N1CCN(c2ccncc2)CC1 ; O=C(C1Cc2cc(Cl)cc(-c3ccnc4[nH]ccc34)c2O1)N1CCC(c2cnc[nH]2)CC1]

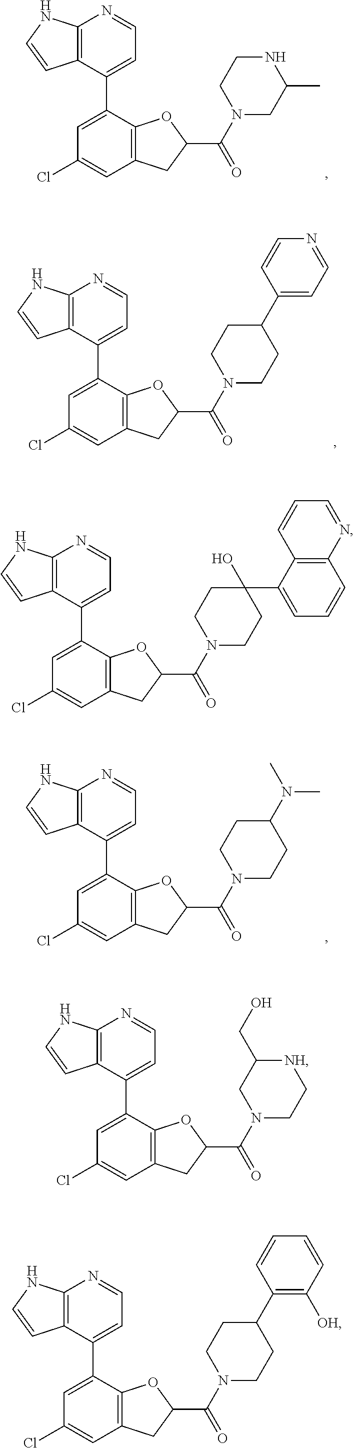 [SMILES: CC1CN(C(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(c2ccncc2)CC1 ; O=C(C1Cc2cc(Cl)cc(-c3ccnc4[nH]ccc34)c2O1)N1CCC(O)(c2cccc3ncccc23)CC1 ; CN(C)C1CCN(C(=O)C2Cc3cc(Cl)cc(-c4ccnc5[nH]ccc45)c3O2)CC1 ; O=C(C1Cc2cc(Cl)cc(-c3ccnc4[nH]ccc34)c2O1)N1CCNC(CO)C1 ; O=C(C1Cc2cc(Cl)cc(-c3ccnc4[nH]ccc34)c2O1)N1CCC(c2ccccc2O)CC1]

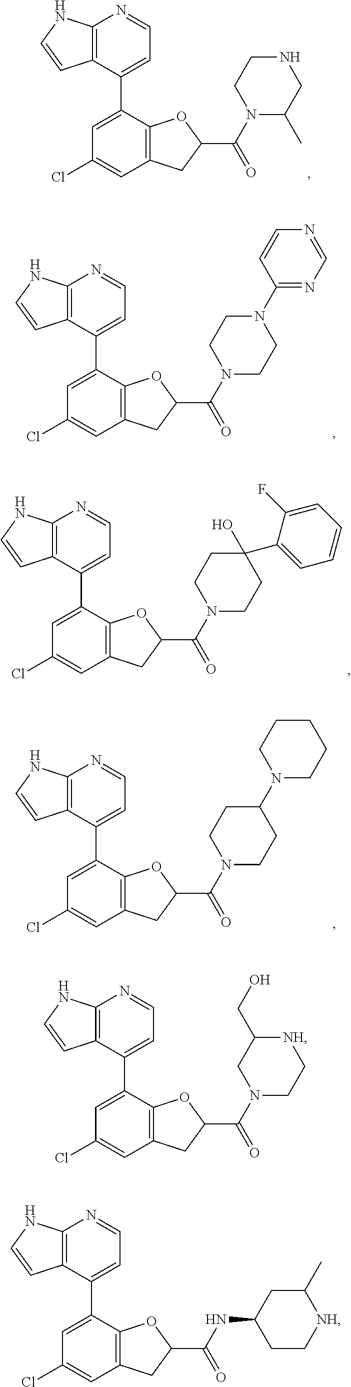 [SMILES: CC1CNCCN1C(=O)C1Cc2cc(Cl)cc(-c3ccnc4[nH]ccc34)c2O1 ; O=C(C1Cc2cc(Cl)cc(-c3ccnc4[nH]ccc34)c2O1)N1CCN(c2ccncn2)CC1 ; O=C(C1Cc2cc(Cl)cc(-c3ccnc4[nH]ccc34)c2O1)N1CCC(O)(c2ccccc2F)CC1 ; O=C(C1Cc2cc(Cl)cc(-c3ccnc4[nH]ccc34)c2O1)N1CCC(N2CCCCC2)CC1 ; O=C(C1Cc2cc(Cl)cc(-c3ccnc4[nH]ccc34)c2O1)N1CCNC(CO)C1 ; CC1C[C@H](NC(=O)C2Cc3cc(Cl)cc(-c4ccnc5[nH]ccc45)c3O2)CCN1]

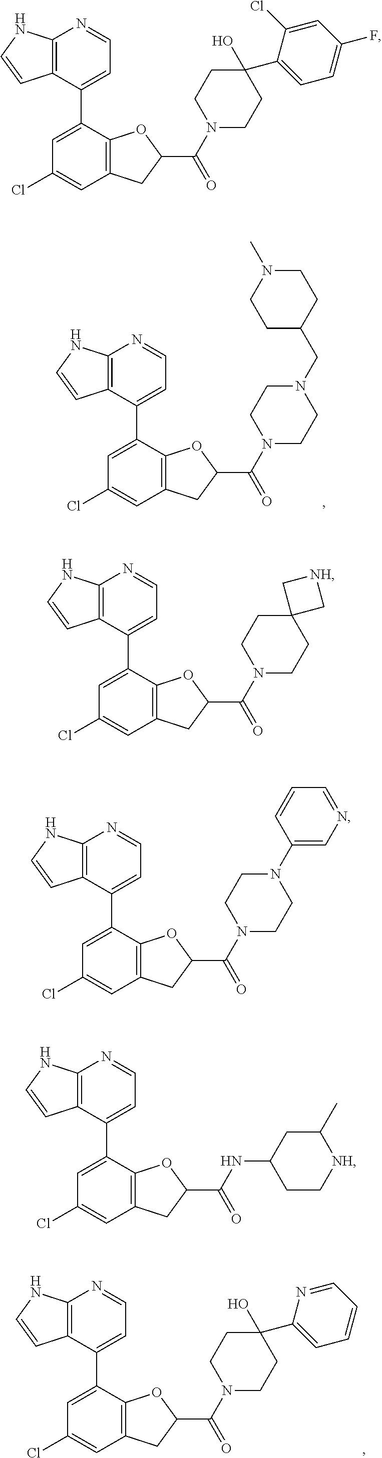 [SMILES: O=C(C1Cc2cc(Cl)cc(-c3ccnc4[nH]ccc34)c2O1)N1CCC(O)(c2ccc(F)cc2Cl)CC1 ; CN1CCC(CN2CCN(C(=O)C3Cc4cc(Cl)cc(-c5ccnc6[nH]ccc56)c4O3)CC2)CC1 ; O=C(C1Cc2cc(Cl)cc(-c3ccnc4[nH]ccc34)c2O1)N1CCC2(CC1)CNC2 ; O=C(C1Cc2cc(Cl)cc(-c3ccnc4[nH]ccc34)c2O1)N1CCN(c2cccnc2)CC1 ; CC1CC(NC(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(O)(c2ccccn2)CC1]

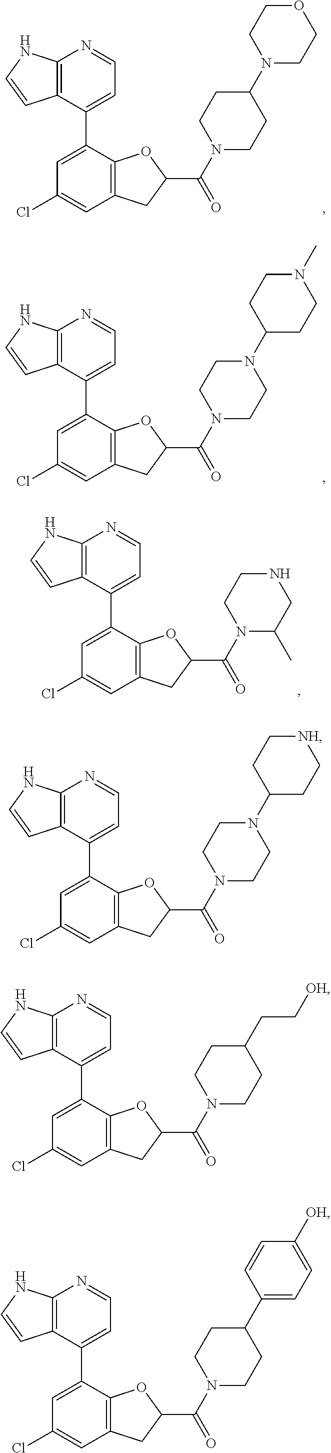 [SMILES: O=C(C1Cc2cc(Cl)cc(-c3ccnc4[nH]ccc34)c2O1)N1CCC(N2CCOCC2)CC1 ; CN1CCC(N2CCN(C(=O)C3Cc4cc(Cl)cc(-c5ccnc6[nH]ccc56)c4O3)CC2)CC1 ; CC1CNCCN1C(=O)C1Cc2cc(Cl)cc(-c3ccnc4[nH]ccc34)c2O1 ; O=C(C1Cc2cc(Cl)cc(-c3ccnc4[nH]ccc34)c2O1)N1CCN(C2CCNCC2)CC1 ; O=C(C1Cc2cc(Cl)cc(-c3ccnc4[nH]ccc34)c2O1)N1CCC(CCO)CC1 ; O=C(C1Cc2cc(Cl)cc(-c3ccnc4[nH]ccc34)c2O1)N1CCC(c2ccc(O)cc2)CC1]

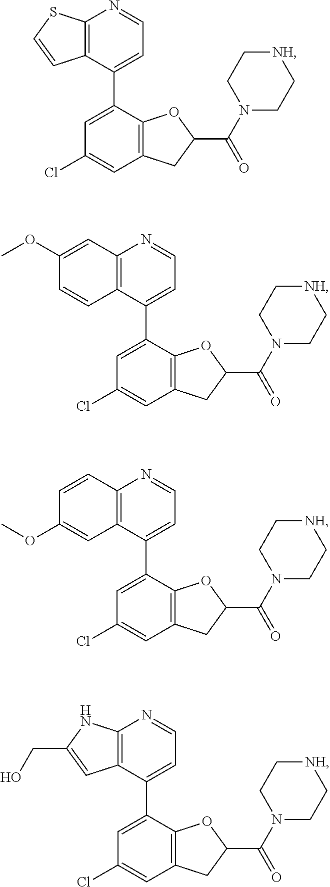 [SMILES: O=C(C1Cc2cc(Cl)cc(-c3ccnc4sccc34)c2O1)N1CCNCC1 ; COc1ccc2c(-c3cc(Cl)cc4c3OC(C(=O)N3CCNCC3)C4)ccnc2c1 ; COc1ccc2nccc(-c3cc(Cl)cc4c3OC(C(=O)N3CCNCC3)C4)c2c1 ; O=C(C1Cc2cc(Cl)cc(-c3ccnc4[nH]c(CO)cc34)c2O1)N1CCNCC1]

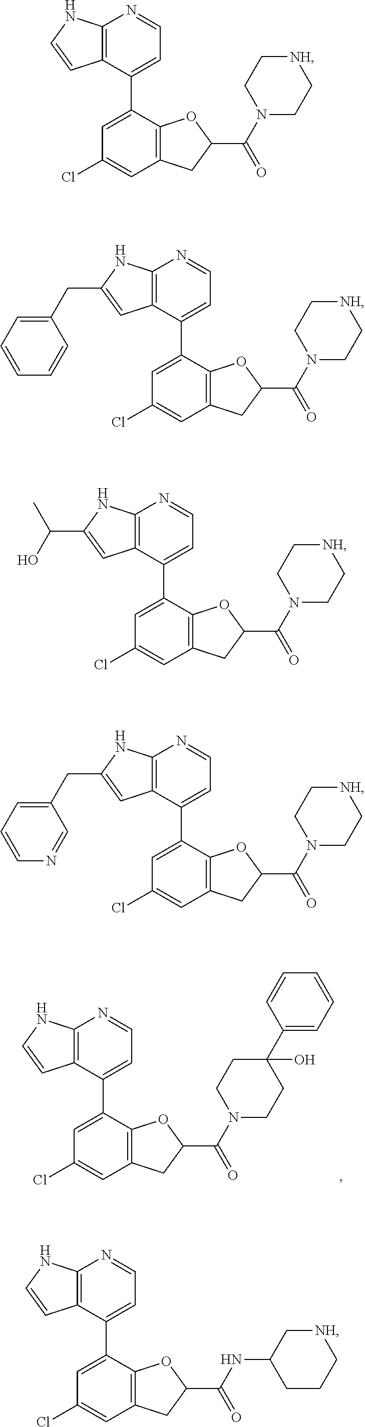 [SMILES: O=C(C1Cc2cc(Cl)cc(-c3ccnc4[nH]ccc34)c2O1)N1CCNCC1 ; O=C(C1Cc2cc(Cl)cc(-c3ccnc4[nH]c(Cc5ccccc5)cc34)c2O1)N1CCNCC1 ; CC(O)c1cc2c(-c3cc(Cl)cc4c3OC(C(=O)N3CCNCC3)C4)ccnc2[nH]1 ; O=C(C1Cc2cc(Cl)cc(-c3ccnc4[nH]c(Cc5cccnc5)cc34)c2O1)N1CCNCC1 ; O=C(C1Cc2cc(Cl)cc(-c3ccnc4[nH]ccc34)c2O1)N1CCC(O)(c2ccccc2)CC1 ; O=C(NC1CCCNC1)C1Cc2cc(Cl)cc(-c3ccnc4[nH]ccc34)c2O1]

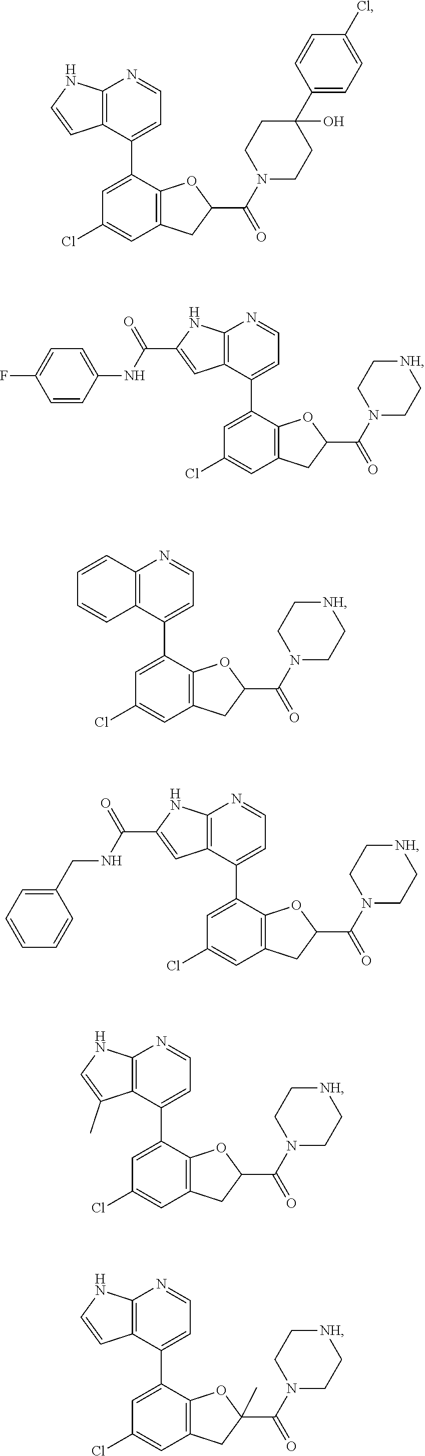 [SMILES: O=C(C1Cc2cc(Cl)cc(-c3ccnc4[nH]ccc34)c2O1)N1CCC(O)(c2ccc(Cl)cc2)CC1 ; O=C(Nc1ccc(F)cc1)c1cc2c(-c3cc(Cl)cc4c3OC(C(=O)N3CCNCC3)C4)ccnc2[nH]1 ; O=C(C1Cc2cc(Cl)cc(-c3ccnc4ccccc34)c2O1)N1CCNCC1 ; O=C(NCc1ccccc1)c1cc2c(-c3cc(Cl)cc4c3OC(C(=O)N3CCNCC3)C4)ccnc2[nH]1 ; Cc1c[nH]c2nccc(-c3cc(Cl)cc4c3OC(C(=O)N3CCNCC3)C4)c12 ; CC1(C(=O)N2CCNCC2)Cc2cc(Cl)cc(-c3ccnc4[nH]ccc34)c2O1]

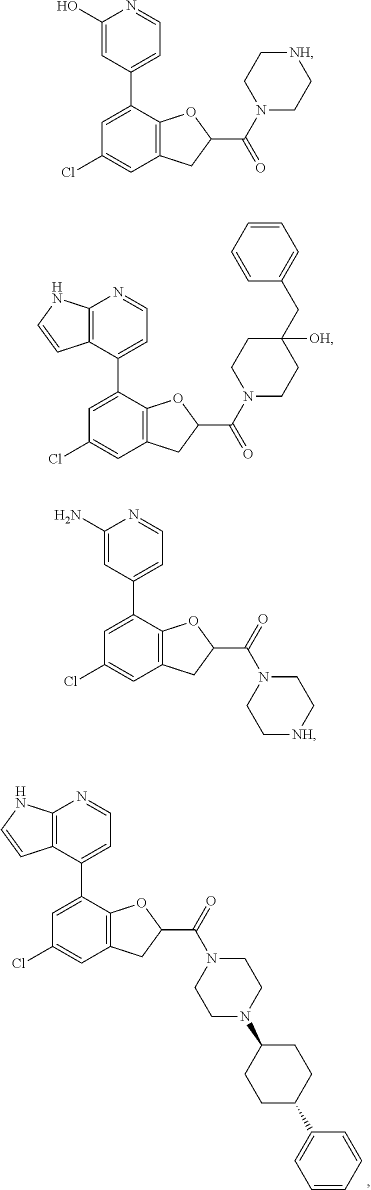 [SMILES: O=C(C1Cc2cc(Cl)cc(-c3ccnc(O)c3)c2O1)N1CCNCC1 ; O=C(C1Cc2cc(Cl)cc(-c3ccnc4[nH]ccc34)c2O1)N1CCC(O)(Cc2ccccc2)CC1 ; Nc1cc(-c2cc(Cl)cc3c2OC(C(=O)N2CCNCC2)C3)ccn1 ; O=C(C1Cc2cc(Cl)cc(-c3ccnc4[nH]ccc34)c2O1)N1CCN([C@H]2CC[C@H](c3ccccc3)CC2)CC1]

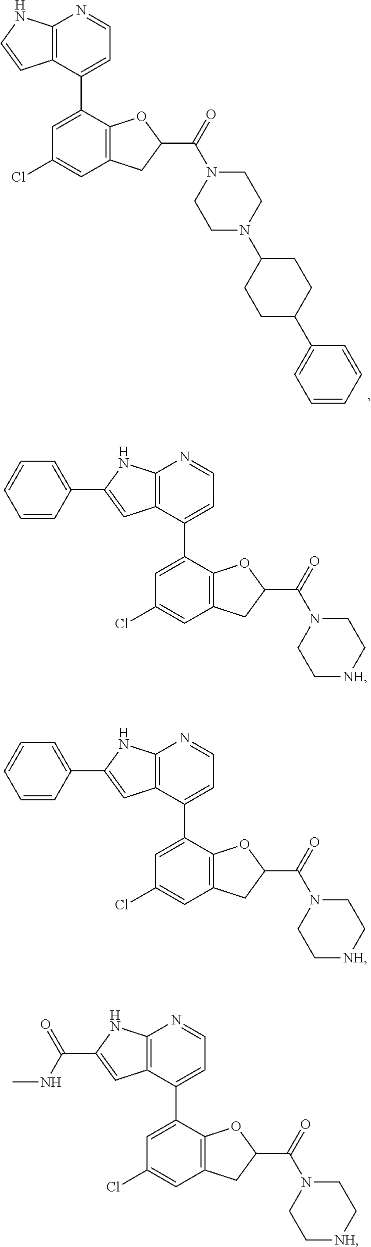 [SMILES: O=C(C1Cc2cc(Cl)cc(-c3ccnc4[nH]ccc34)c2O1)N1CCN(C2CCC(c3ccccc3)CC2)CC1 ; O=C(C1Cc2cc(Cl)cc(-c3ccnc4[nH]c(-c5ccccc5)cc34)c2O1)N1CCNCC1 ; O=C(C1Cc2cc(Cl)cc(-c3ccnc4[nH]c(-c5ccccc5)cc34)c2O1)N1CCNCC1 ; CNC(=O)c1cc2c(-c3cc(Cl)cc4c3OC(C(=O)N3CCNCC3)C4)ccnc2[nH]1]

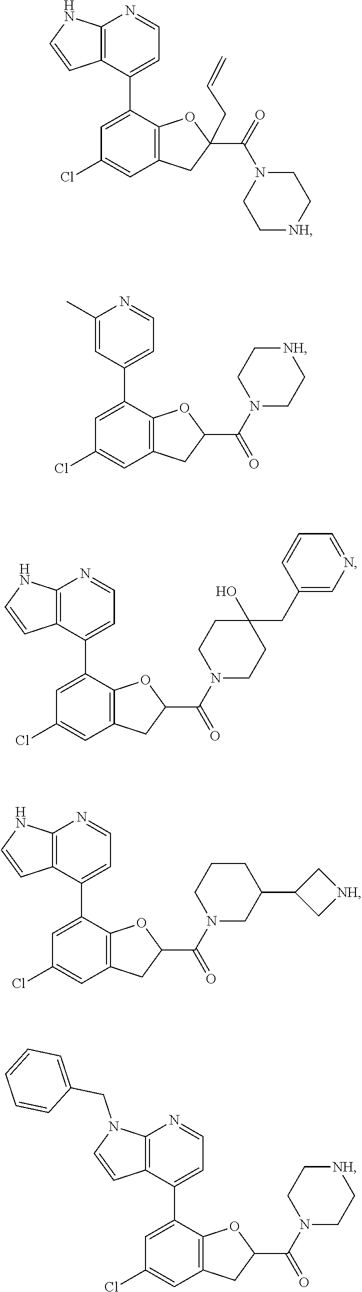 [SMILES: C=CCC1(C(=O)N2CCNCC2)Cc2cc(Cl)cc(-c3ccnc4[nH]ccc34)c2O1 ; Cc1cc(-c2cc(Cl)cc3c2OC(C(=O)N2CCNCC2)C3)ccn1 ; O=C(C1Cc2cc(Cl)cc(-c3ccnc4[nH]ccc34)c2O1)N1CCC(O)(Cc2cccnc2)CC1 ; O=C(C1Cc2cc(Cl)cc(-c3ccnc4[nH]ccc34)c2O1)N1CCCC(C2CNC2)C1 ; O=C(C1Cc2cc(Cl)cc(-c3ccnc4c3ccn4Cc3ccccc3)c2O1)N1CCNCC1]

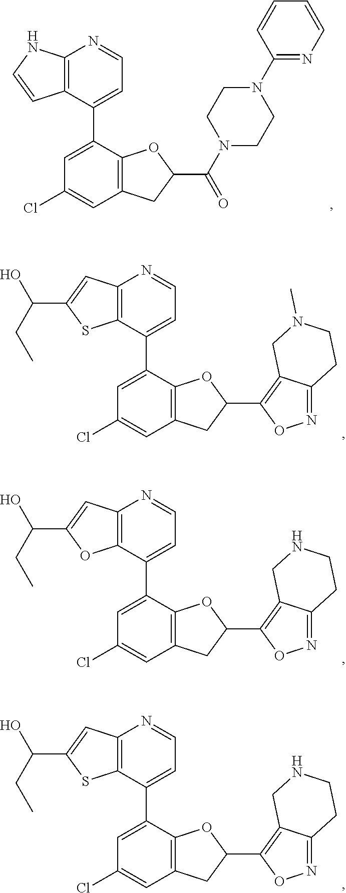 [SMILES: O=C(C1Cc2cc(Cl)cc(-c3ccnc4[nH]ccc34)c2O1)N1CCN(c2ccccn2)CC1 ; CCC(O)c1cc2nccc(-c3cc(Cl)cc4c3OC(c3onc5c3CN(C)CC5)C4)c2s1 ; CCC(O)c1cc2nccc(-c3cc(Cl)cc4c3OC(c3onc5c3CNCC5)C4)c2o1 ; CCC(O)c1cc2nccc(-c3cc(Cl)cc4c3OC(c3onc5c3CNCC5)C4)c2s1]

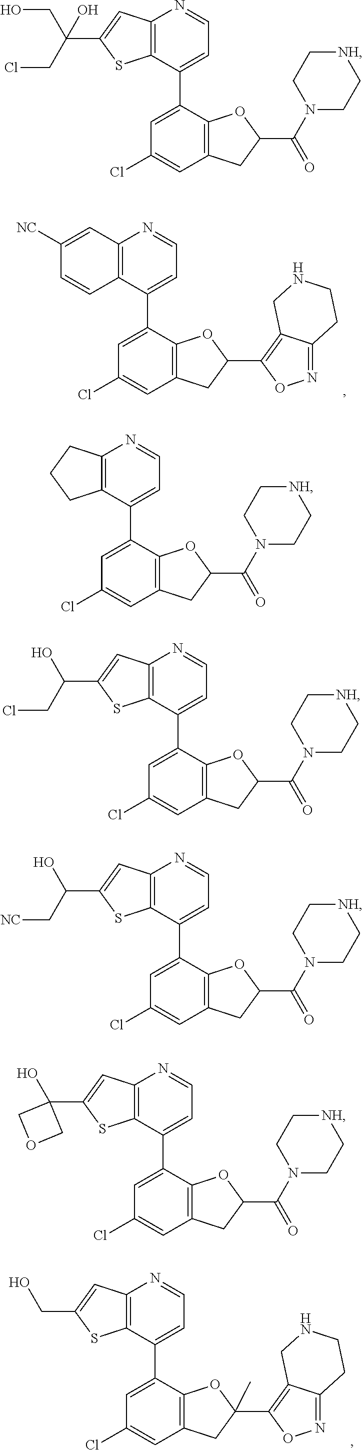

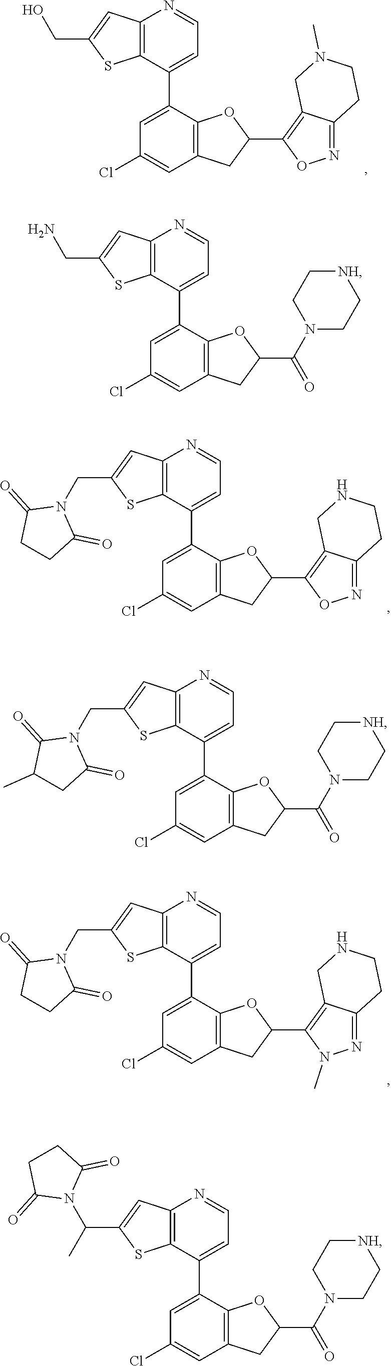 [SMILES: CN1CCc2noc(C3Cc4cc(Cl)cc(-c5ccnc6cc(CO)sc56)c4O3)c2C1 ; NCc1cc2nccc(-c3cc(Cl)cc4c3OC(C(=O)N3CCNCC3)C4)c2s1 ; O=C1CCC(=O)N1Cc1cc2nccc(-c3cc(Cl)cc4c3OC(c3onc5c3CNCC5)C4)c2s1 ; CC1CC(=O)N(Cc2cc3nccc(-c4cc(Cl)cc5c4OC(C(=O)N4CCNCC4)C5)c3s2)C1=O ; Cn1nc2c(c1C1Cc3cc(Cl)cc(-c4ccnc5cc(CN6C(=O)CCC6=O)sc45)c3O1)CNCC2 ; CC(c1cc2nccc(-c3cc(Cl)cc4c3OC(C(=O)N3CCNCC3)C4)c2s1)N1C(=O)CCC1=O]

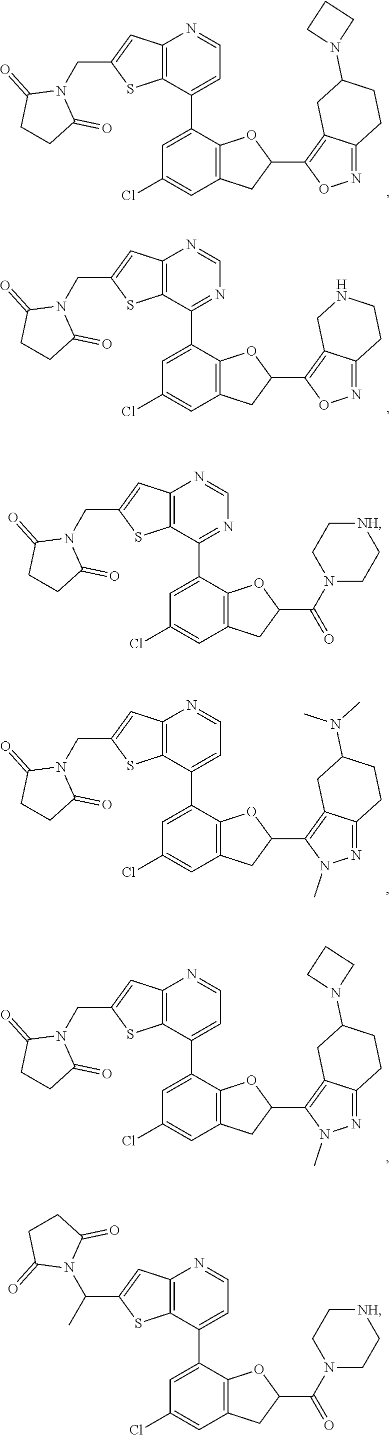 [SMILES: O=C1CCC(=O)N1Cc1cc2nccc(-c3cc(Cl)cc4c3OC(c3onc5c3CC(N3CCC3)CC5)C4)c2s1 ; O=C1CCC(=O)N1Cc1cc2ncnc(-c3cc(Cl)cc4c3OC(c3onc5c3CNCC5)C4)c2s1 ; O=C(C1Cc2cc(Cl)cc(-c3ncnc4cc(CN5C(=O)CCC5=O)sc34)c2O1)N1CCNCC1 ; CN(C)C1CCc2nn(C)c(C3Cc4cc(Cl)cc(-c5ccnc6cc(CN7C(=O)CCC7=O)sc56)c4O3)c2C1 ; Cn1nc2c(c1C1Cc3cc(Cl)cc(-c4ccnc5cc(CN6C(=O)CCC6=O)sc45)c3O1)CC(N1CCC1)CC2 ; CC(c1cc2nccc(-c3cc(Cl)cc4c3OC(C(=O)N3CCNCC3)C4)c2s1)N1C(=O)CCC1=O]

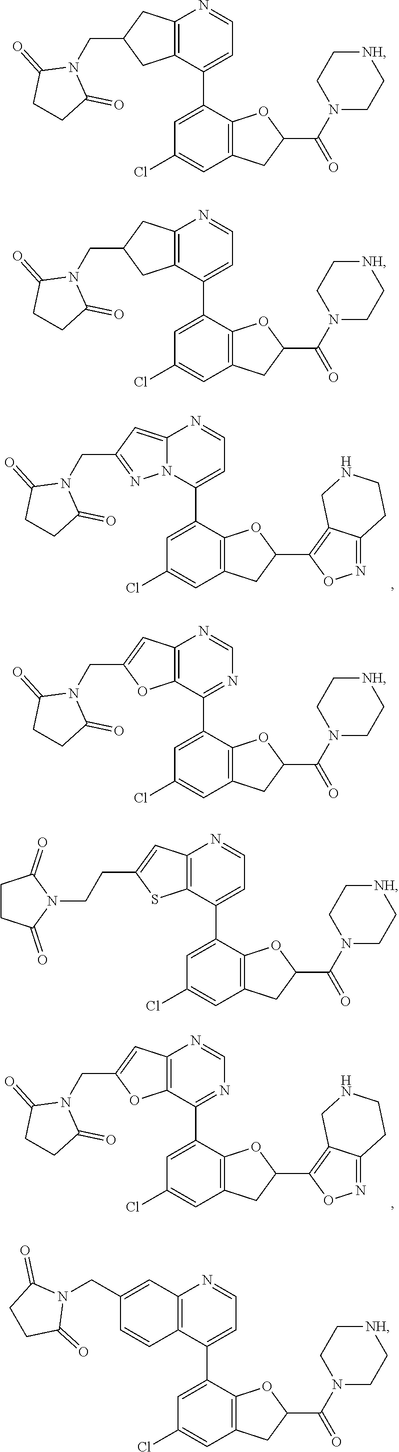

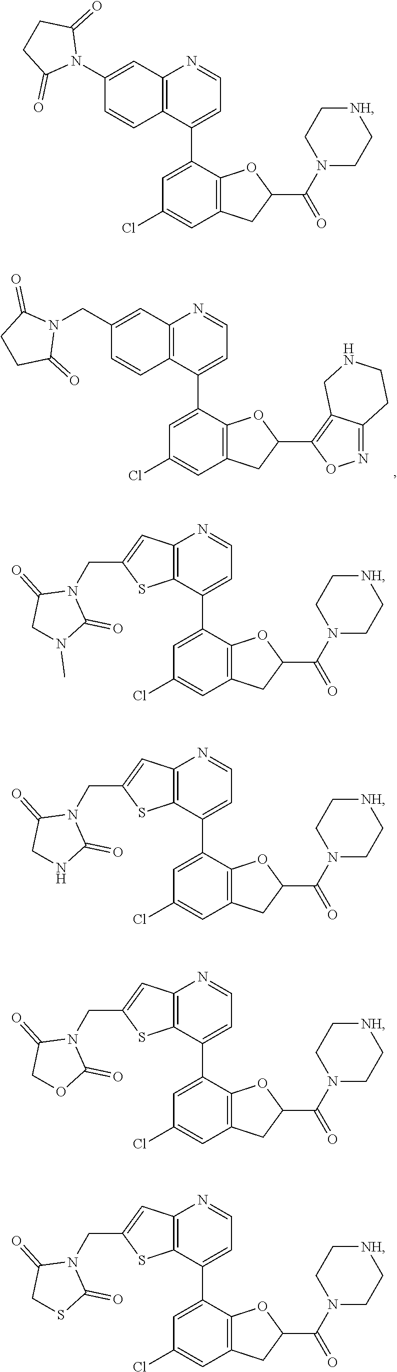 [SMILES: O=C(C1Cc2cc(Cl)cc(-c3ccnc4cc(N5C(=O)CCC5=O)ccc34)c2O1)N1CCNCC1 ; O=C1CCC(=O)N1Cc1ccc2c(-c3cc(Cl)cc4c3OC(c3onc5c3CNCC5)C4)ccnc2c1 ; CN1CC(=O)N(Cc2cc3nccc(-c4cc(Cl)cc5c4OC(C(=O)N4CCNCC4)C5)c3s2)C1=O ; O=C(C1Cc2cc(Cl)cc(-c3ccnc4cc(CN5C(=O)CNC5=O)sc34)c2O1)N1CCNCC1 ; O=C(C1Cc2cc(Cl)cc(-c3ccnc4cc(CN5C(=O)COC5=O)sc34)c2O1)N1CCNCC1 ; O=C(C1Cc2cc(Cl)cc(-c3ccnc4cc(CN5C(=O)CSC5=O)sc34)c2O1)N1CCNCC1]

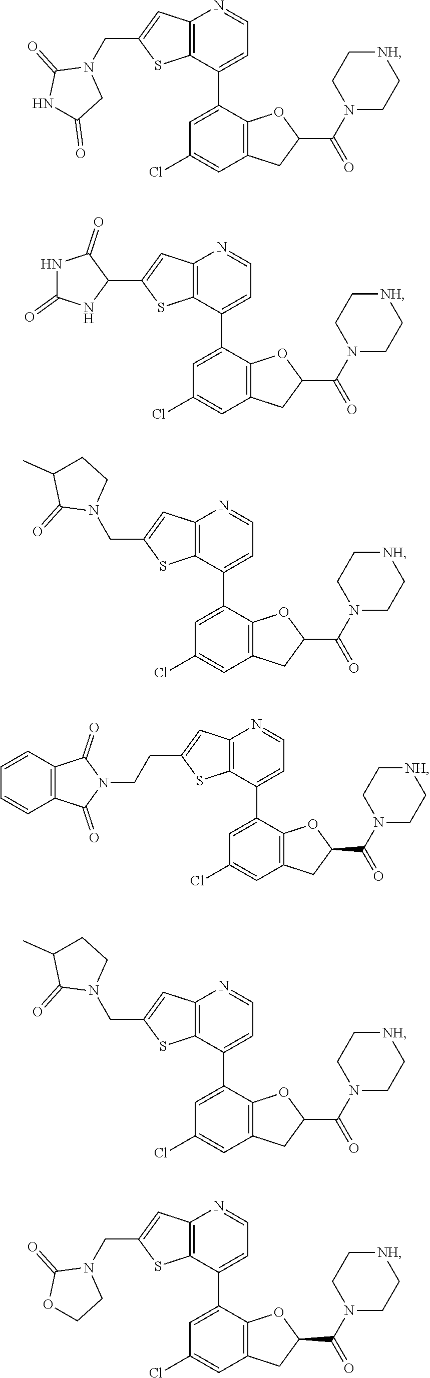 [SMILES: O=C1CN(Cc2cc3nccc(-c4cc(Cl)cc5c4OC(C(=O)N4CCNCC4)C5)c3s2)C(=O)N1 ; O=C1NC(=O)C(c2cc3nccc(-c4cc(Cl)cc5c4OC(C(=O)N4CCNCC4)C5)c3s2)N1 ; CC1CCN(Cc2cc3nccc(-c4cc(Cl)cc5c4OC(C(=O)N4CCNCC4)C5)c3s2)C1=O ; O=C([C@H]1Cc2cc(Cl)cc(-c3ccnc4cc(CCN5C(=O)c6ccccc6C5=O)sc34)c2O1)N1CCNCC1 ; CC1CCN(Cc2cc3nccc(-c4cc(Cl)cc5c4OC(C(=O)N4CCNCC4)C5)c3s2)C1=O ; O=C1OCCN1Cc1cc2nccc(-c3cc(Cl)cc4c3O[C@@H](C(=O)N3CCNCC3)C4)c2s1]

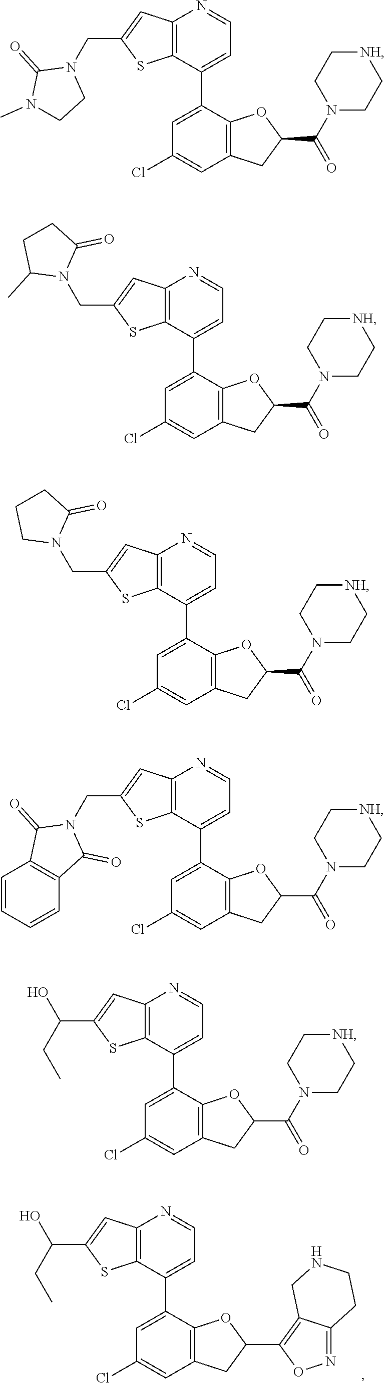 [SMILES: CN1CCN(Cc2cc3nccc(-c4cc(Cl)cc5c4O[C@@H](C(=O)N4CCNCC4)C5)c3s2)C1=O ; CC1CCC(=O)N1Cc1cc2nccc(-c3cc(Cl)cc4c3O[C@@H](C(=O)N3CCNCC3)C4)c2s1 ; O=C1CCCN1Cc1cc2nccc(-c3cc(Cl)cc4c3O[C@@H](C(=O)N3CCNCC3)C4)c2s1 ; O=C(C1Cc2cc(Cl)cc(-c3ccnc4cc(CN5C(=O)c6ccccc6C5=O)sc34)c2O1)N1CCNCC1 ; CCC(O)c1cc2nccc(-c3cc(Cl)cc4c3OC(C(=O)N3CCNCC3)C4)c2s1 ; CCC(O)c1cc2nccc(-c3cc(Cl)cc4c3OC(c3onc5c3CNCC5)C4)c2s1]

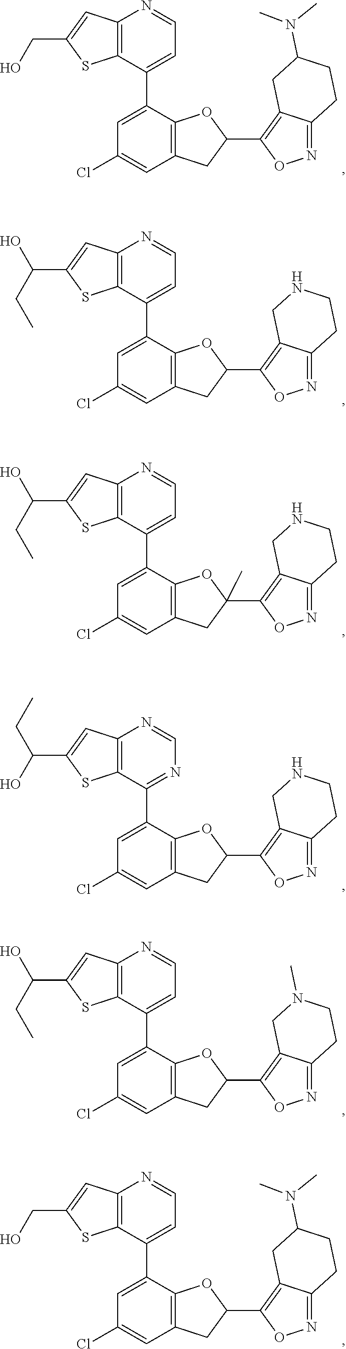 [SMILES: CN(C)C1CCc2noc(C3Cc4cc(Cl)cc(-c5ccnc6cc(CO)sc56)c4O3)c2C1 ; CCC(O)c1cc2nccc(-c3cc(Cl)cc4c3OC(c3onc5c3CNCC5)C4)c2s1 ; CCC(O)c1cc2nccc(-c3cc(Cl)cc4c3OC(C)(c3onc5c3CNCC5)C4)c2s1 ; CCC(O)c1cc2ncnc(-c3cc(Cl)cc4c3OC(c3onc5c3CNCC5)C4)c2s1 ; CCC(O)c1cc2nccc(-c3cc(Cl)cc4c3OC(c3onc5c3CN(C)CC5)C4)c2s1 ; CN(C)C1CCc2noc(C3Cc4cc(Cl)cc(-c5ccnc6cc(CO)sc56)c4O3)c2C1]

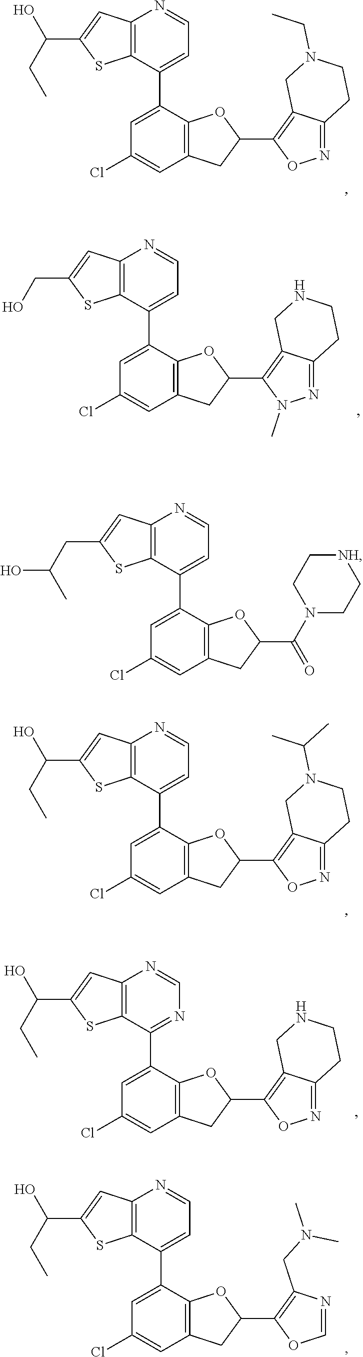 [SMILES: CCC(O)c1cc2nccc(-c3cc(Cl)cc4c3OC(c3onc5c3CN(CC)CC5)C4)c2s1 ; Cn1nc2c(c1C1Cc3cc(Cl)cc(-c4ccnc5cc(CO)sc45)c3O1)CNCC2 ; CC(O)Cc1cc2nccc(-c3cc(Cl)cc4c3OC(C(=O)N3CCNCC3)C4)c2s1 ; CCC(O)c1cc2nccc(-c3cc(Cl)cc4c3OC(c3onc5c3CN(C(C)C)CC5)C4)c2s1 ; CCC(O)c1cc2ncnc(-c3cc(Cl)cc4c3OC(c3onc5c3CNCC5)C4)c2s1 ; CCC(O)c1cc2nccc(-c3cc(Cl)cc4c3OC(c3ocnc3CN(C)C)C4)c2s1]

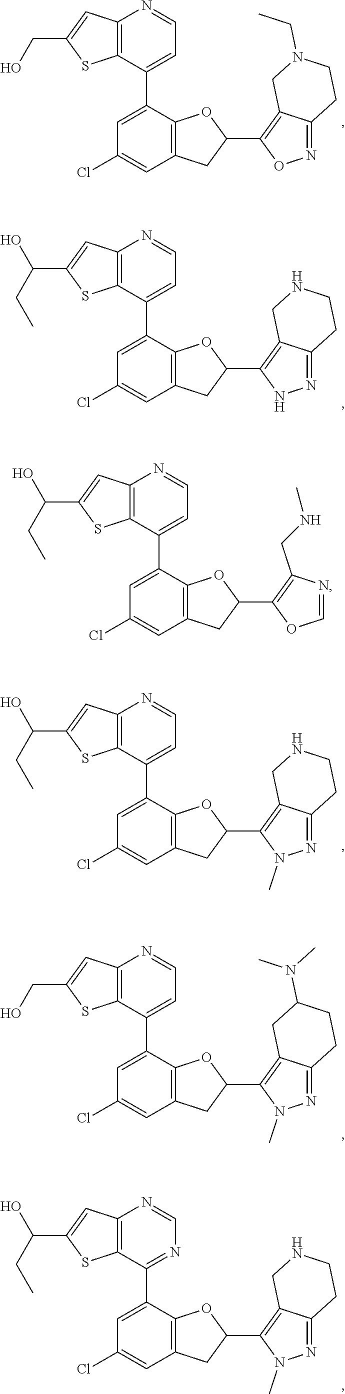 [SMILES: CCN1CCc2noc(C3Cc4cc(Cl)cc(-c5ccnc6cc(CO)sc56)c4O3)c2C1 ; CCC(O)c1cc2nccc(-c3cc(Cl)cc4c3OC(c3[nH]nc5c3CNCC5)C4)c2s1 ; CCC(O)c1cc2nccc(-c3cc(Cl)cc4c3OC(c3ocnc3CNC)C4)c2s1 ; CCC(O)c1cc2nccc(-c3cc(Cl)cc4c3OC(c3c5c(nn3C)CCNC5)C4)c2s1 ; CN(C)C1CCc2nn(C)c(C3Cc4cc(Cl)cc(-c5ccnc6cc(CO)sc56)c4O3)c2C1 ; CCC(O)c1cc2ncnc(-c3cc(Cl)cc4c3OC(c3c5c(nn3C)CCNC5)C4)c2s1]

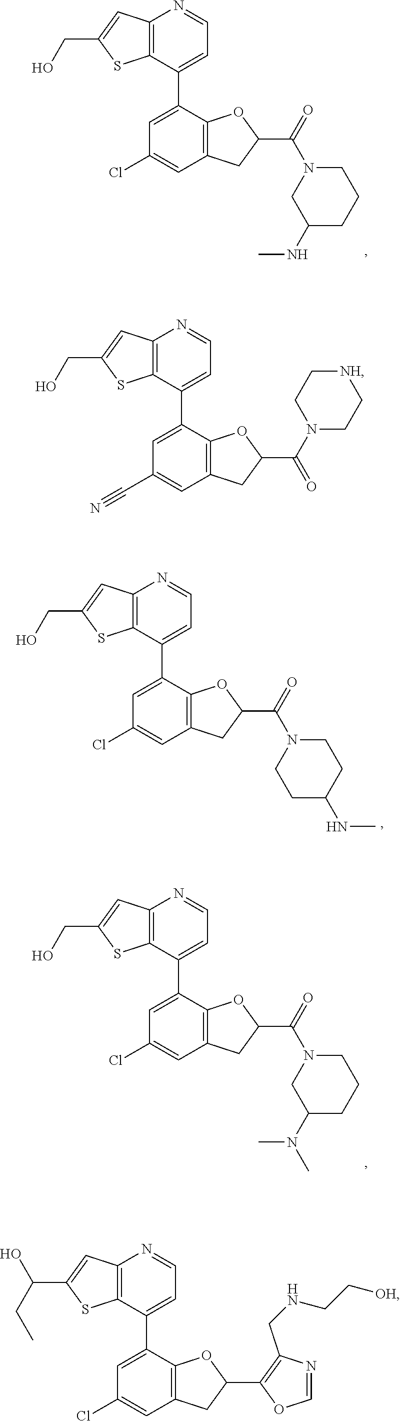 [SMILES: CNC1CCCN(C(=O)C2Cc3cc(Cl)cc(-c4ccnc5cc(CO)sc45)c3O2)C1 ; N#Cc1cc2c(c(-c3ccnc4cc(CO)sc34)c1)OC(C(=O)N1CCNCC1)C2 ; CNC1CCN(C(=O)C2Cc3cc(Cl)cc(-c4ccnc5cc(CO)sc45)c3O2)CC1 ; CN(C)C1CCCN(C(=O)C2Cc3cc(Cl)cc(-c4ccnc5cc(CO)sc45)c3O2)C1 ; CCC(O)c1cc2nccc(-c3cc(Cl)cc4c3OC(c3ocnc3CNCCO)C4)c2s1]

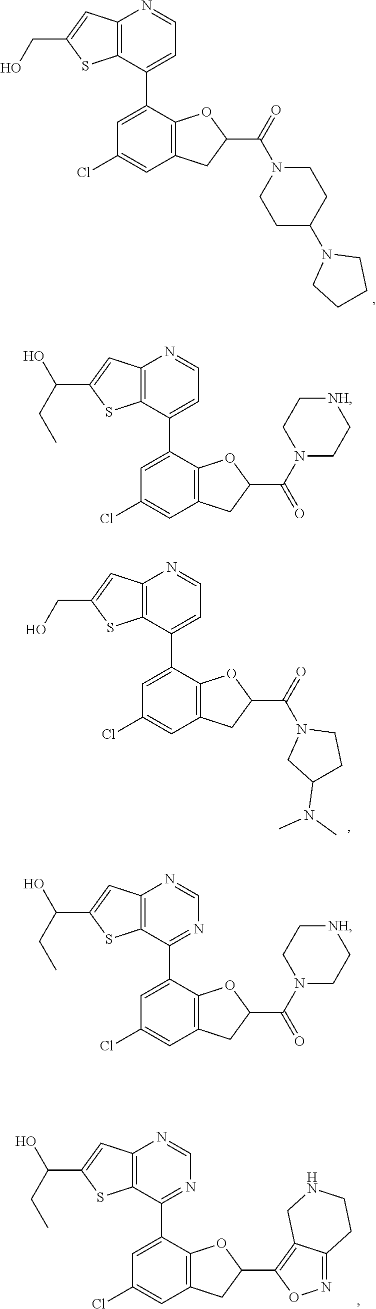 [SMILES: O=C(C1Cc2cc(Cl)cc(-c3ccnc4cc(CO)sc34)c2O1)N1CCC(N2CCCC2)CC1 ; CCC(O)c1cc2nccc(-c3cc(Cl)cc4c3OC(C(=O)N3CCNCC3)C4)c2s1 ; CN(C)C1CCN(C(=O)C2Cc3cc(Cl)cc(-c4ccnc5cc(CO)sc45)c3O2)C1 ; CCC(O)c1cc2ncnc(-c3cc(Cl)cc4c3OC(C(=O)N3CCNCC3)C4)c2s1 ; CCC(O)c1cc2ncnc(-c3cc(Cl)cc4c3OC(c3onc5c3CNCC5)C4)c2s1]

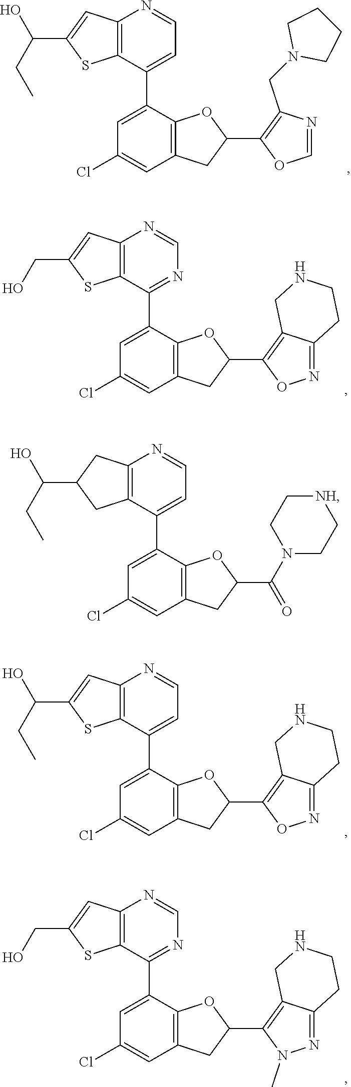 [SMILES: CCC(O)c1cc2nccc(-c3cc(Cl)cc4c3OC(c3ocnc3CN3CCCC3)C4)c2s1 ; OCc1cc2ncnc(-c3cc(Cl)cc4c3OC(c3onc5c3CNCC5)C4)c2s1 ; CCC(O)C1Cc2nccc(-c3cc(Cl)cc4c3OC(C(=O)N3CCNCC3)C4)c2C1 ; CCC(O)c1cc2nccc(-c3cc(Cl)cc4c3OC(c3onc5c3CNCC5)C4)c2s1 ; Cn1nc2c(c1C1Cc3cc(Cl)cc(-c4ncnc5cc(CO)sc45)c3O1)CNCC2]

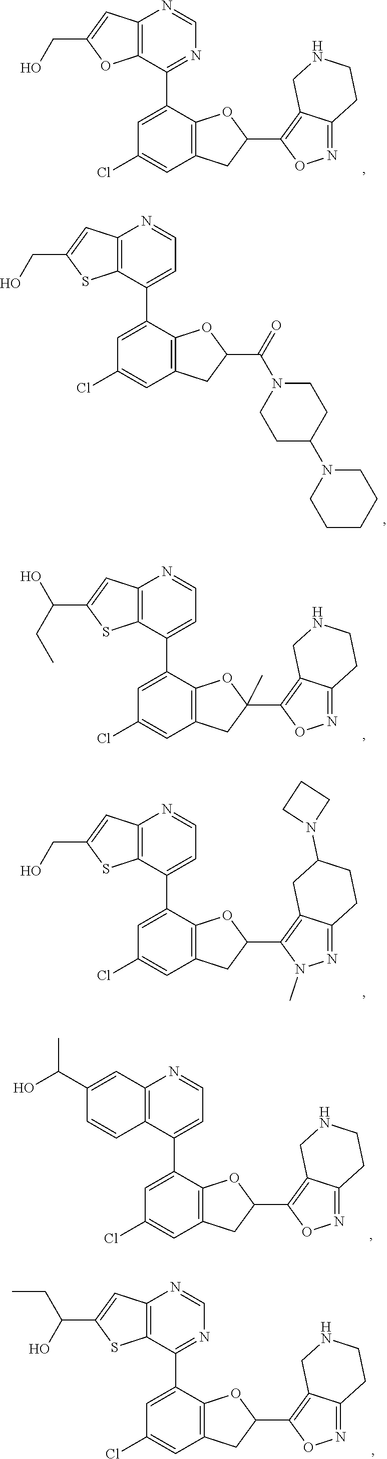 [SMILES: OCc1cc2ncnc(-c3cc(Cl)cc4c3OC(c3onc5c3CNCC5)C4)c2o1 ; O=C(C1Cc2cc(Cl)cc(-c3ccnc4cc(CO)sc34)c2O1)N1CCC(N2CCCCC2)CC1 ; CCC(O)c1cc2nccc(-c3cc(Cl)cc4c3OC(C)(c3onc5c3CNCC5)C4)c2s1 ; Cn1nc2c(c1C1Cc3cc(Cl)cc(-c4ccnc5cc(CO)sc45)c3O1)CC(N1CCC1)CC2 ; CC(O)c1ccc2c(-c3cc(Cl)cc4c3OC(c3onc5c3CNCC5)C4)ccnc2c1 ; CCC(O)c1cc2ncnc(-c3cc(Cl)cc4c3OC(c3onc5c3CNCC5)C4)c2s1]

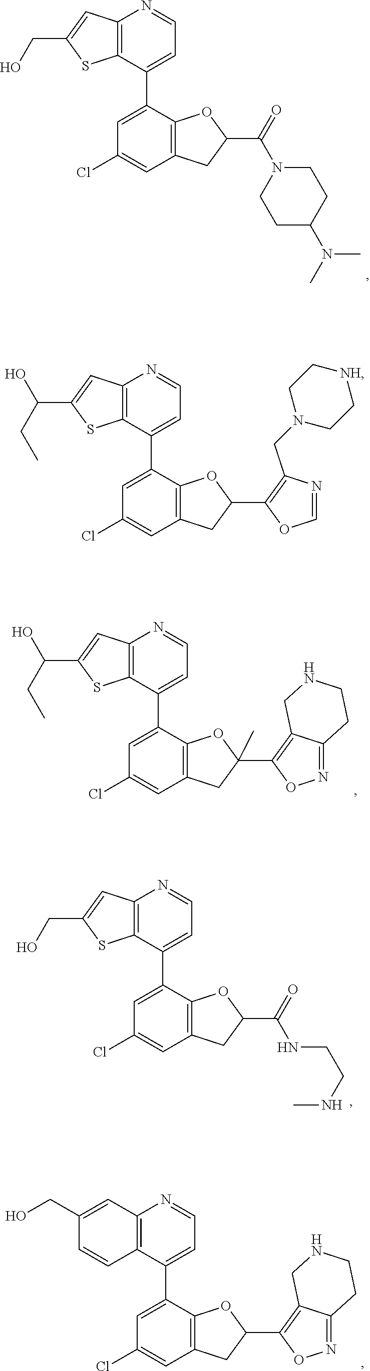 [SMILES: CN(C)C1CCN(C(=O)C2Cc3cc(Cl)cc(-c4ccnc5cc(CO)sc45)c3O2)CC1 ; CCC(O)c1cc2nccc(-c3cc(Cl)cc4c3OC(c3ocnc3CN3CCNCC3)C4)c2s1 ; CCC(O)c1cc2nccc(-c3cc(Cl)cc4c3OC(C)(c3onc5c3CNCC5)C4)c2s1 ; CNCCNC(=O)C1Cc2cc(Cl)cc(-c3ccnc4cc(CO)sc34)c2O1 ; OCc1ccc2c(-c3cc(Cl)cc4c3OC(c3onc5c3CNCC5)C4)ccnc2c1]

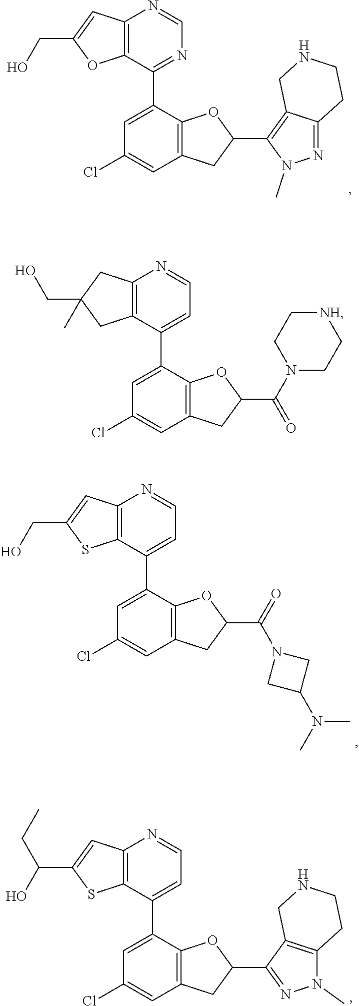 [SMILES: Cn1nc2c(c1C1Cc3cc(Cl)cc(-c4ncnc5cc(CO)oc45)c3O1)CNCC2 ; CC1(CO)Cc2nccc(-c3cc(Cl)cc4c3OC(C(=O)N3CCNCC3)C4)c2C1 ; CN(C)C1CN(C(=O)C2Cc3cc(Cl)cc(-c4ccnc5cc(CO)sc45)c3O2)C1 ; CCC(O)c1cc2nccc(-c3cc(Cl)cc4c3OC(c3nn(C)c5c3CNCC5)C4)c2s1]

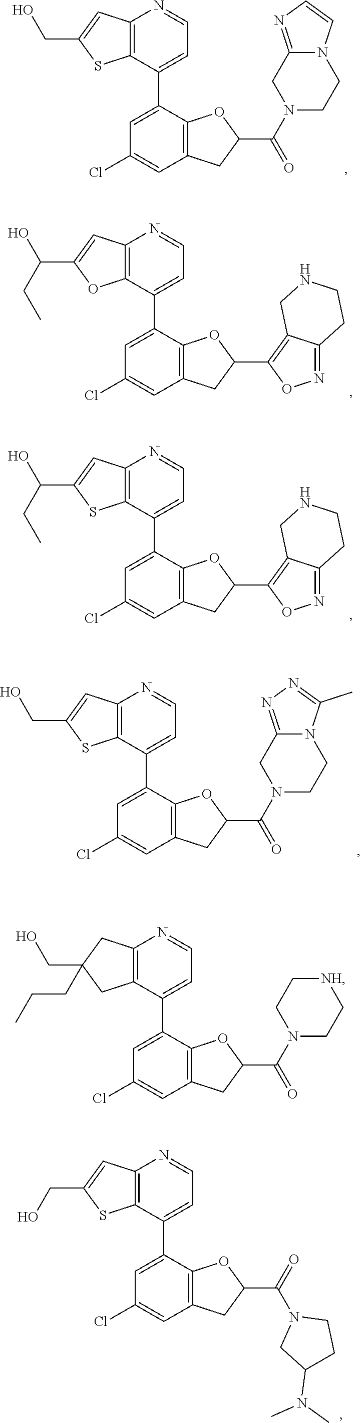

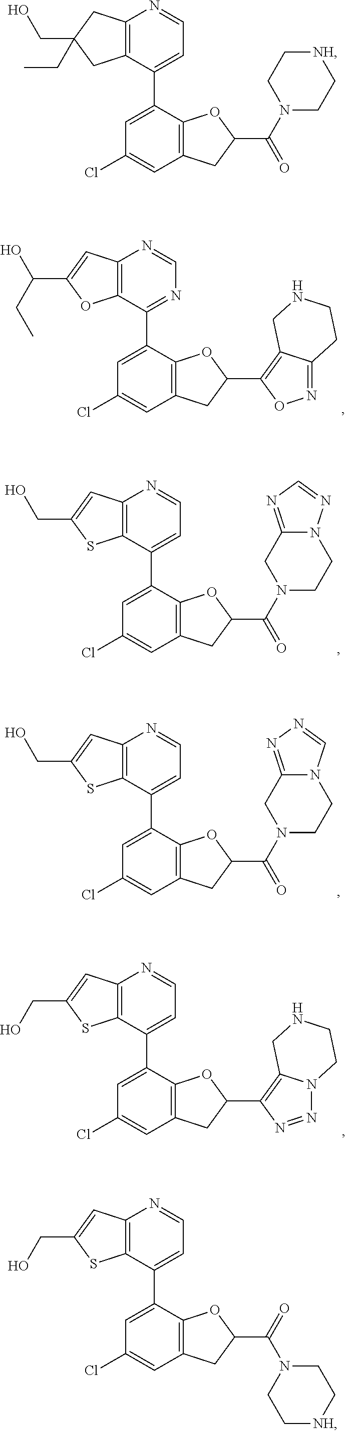 [SMILES: CCC1(CO)Cc2nccc(-c3cc(Cl)cc4c3OC(C(=O)N3CCNCC3)C4)c2C1 ; CCC(O)c1cc2ncnc(-c3cc(Cl)cc4c3OC(c3onc5c3CNCC5)C4)c2o1 ; O=C(C1Cc2cc(Cl)cc(-c3ccnc4cc(CO)sc34)c2O1)N1CCn2ncnc2C1 ; O=C(C1Cc2cc(Cl)cc(-c3ccnc4cc(CO)sc34)c2O1)N1CCn2cnnc2C1 ; OCc1cc2nccc(-c3cc(Cl)cc4c3OC(c3nnn5c3CNCC5)C4)c2s1 ; O=C(C1Cc2cc(Cl)cc(-c3ccnc4cc(CO)sc34)c2O1)N1CCNCC1]

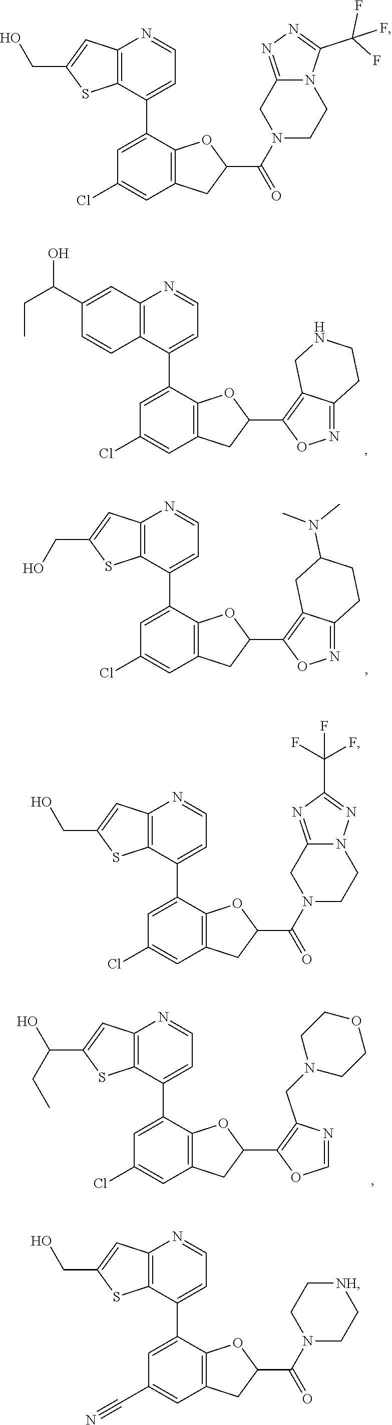 [SMILES: O=C(C1Cc2cc(Cl)cc(-c3ccnc4cc(CO)sc34)c2O1)N1CCn2c(nnc2C(F)(F)F)C1 ; CCC(O)c1ccc2c(-c3cc(Cl)cc4c3OC(c3onc5c3CNCC5)C4)ccnc2c1 ; CN(C)C1CCc2noc(C3Cc4cc(Cl)cc(-c5ccnc6cc(CO)sc56)c4O3)c2C1 ; O=C(C1Cc2cc(Cl)cc(-c3ccnc4cc(CO)sc34)c2O1)N1CCn2nc(C(F)(F)F)nc2C1 ; CCC(O)c1cc2nccc(-c3cc(Cl)cc4c3OC(c3ocnc3CN3CCOCC3)C4)c2s1 ; N#Cc1cc2c(c(-c3ccnc4cc(CO)sc34)c1)OC(C(=O)N1CCNCC1)C2]

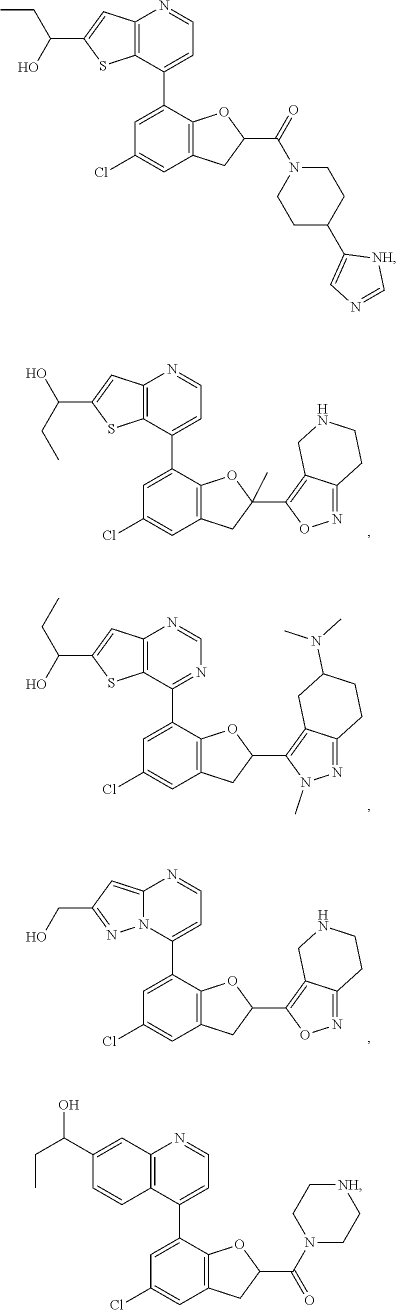 [SMILES: CCC(O)c1cc2nccc(-c3cc(Cl)cc4c3OC(C(=O)N3CCC(c5cnc[nH]5)CC3)C4)c2s1 ; CCC(O)c1cc2nccc(-c3cc(Cl)cc4c3OC(C)(c3onc5c3CNCC5)C4)c2s1 ; CCC(O)c1cc2ncnc(-c3cc(Cl)cc4c3OC(c3c5c(nn3C)CCC(N(C)C)C5)C4)c2s1 ; OCc1cc2nccc(-c3cc(Cl)cc4c3OC(c3onc5c3CNCC5)C4)n2n1 ; CCC(O)c1ccc2c(-c3cc(Cl)cc4c3OC(C(=O)N3CCNCC3)C4)ccnc2c1]

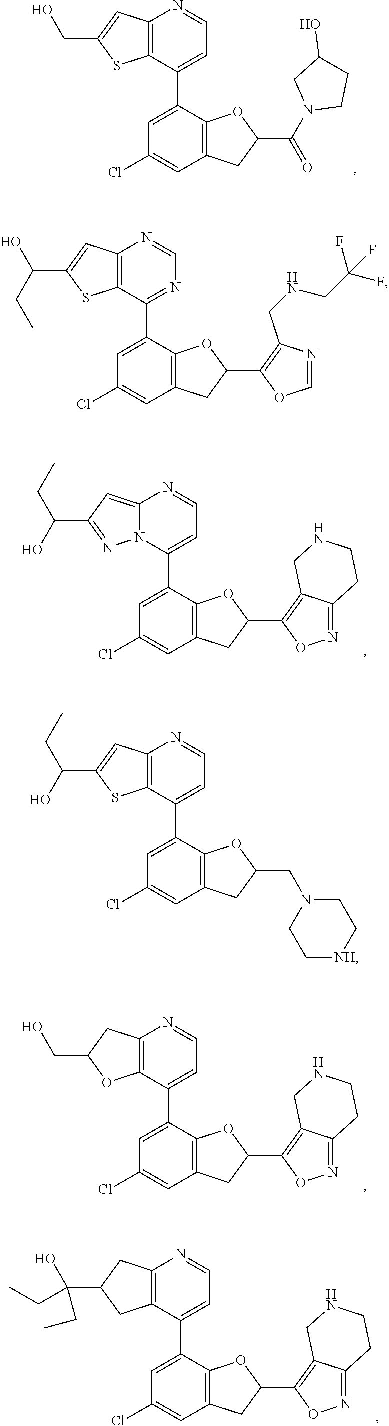 [SMILES: O=C(C1Cc2cc(Cl)cc(-c3ccnc4cc(CO)sc34)c2O1)N1CCC(O)C1 ; CCC(O)c1cc2ncnc(-c3cc(Cl)cc4c3OC(c3ocnc3CNCC(F)(F)F)C4)c2s1 ; CCC(O)c1cc2nccc(-c3cc(Cl)cc4c3OC(c3onc5c3CNCC5)C4)n2n1 ; CCC(O)c1cc2nccc(-c3cc(Cl)cc4c3OC(CN3CCNCC3)C4)c2s1 ; OCC1Cc2nccc(-c3cc(Cl)cc4c3OC(c3onc5c3CNCC5)C4)c2O1 ; CCC(O)(CC)C1Cc2nccc(-c3cc(Cl)cc4c3OC(c3onc5c3CNCC5)C4)c2C1]

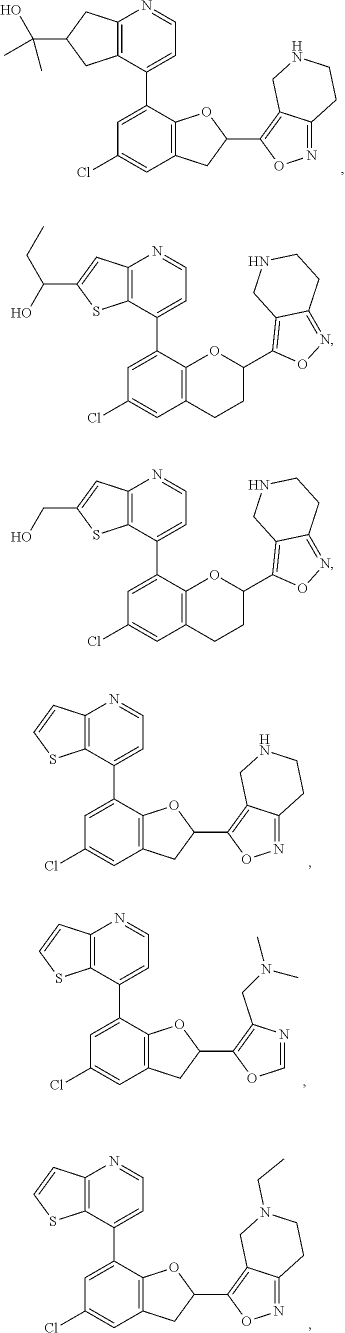 [SMILES: CC(C)(O)C1Cc2nccc(-c3cc(Cl)cc4c3OC(c3onc5c3CNCC5)C4)c2C1 ; CCC(O)c1cc2nccc(-c3cc(Cl)cc4c3OC(c3onc5c3CNCC5)CC4)c2s1 ; OCc1cc2nccc(-c3cc(Cl)cc4c3OC(c3onc5c3CNCC5)CC4)c2s1 ; Clc1cc2c(c(-c3ccnc4ccsc34)c1)OC(c1onc3c1CNCC3)C2 ; CN(C)Cc1ncoc1C1Cc2cc(Cl)cc(-c3ccnc4ccsc34)c2O1 ; CCN1CCc2noc(C3Cc4cc(Cl)cc(-c5ccnc6ccsc56)c4O3)c2C1]

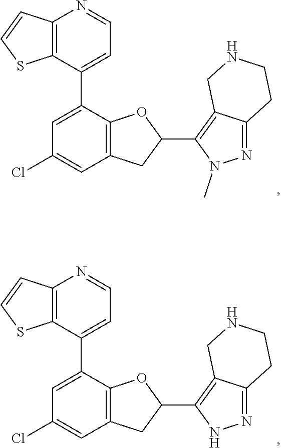 [SMILES: Cn1nc2c(c1C1Cc3cc(Cl)cc(-c4ccnc5ccsc45)c3O1)CNCC2 ; Clc1cc2c(c(-c3ccnc4ccsc34)c1)OC(c1[nH]nc3c1CNCC3)C2]

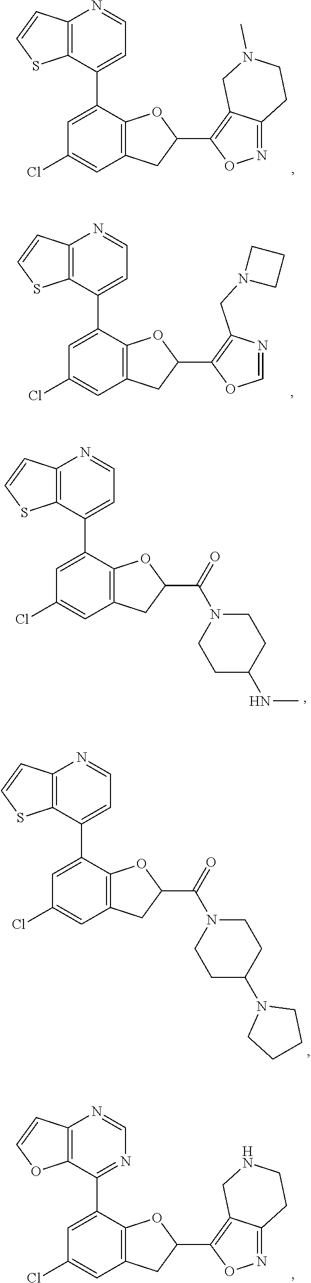 [SMILES: CN1CCc2noc(C3Cc4cc(Cl)cc(-c5ccnc6ccsc56)c4O3)c2C1 ; Clc1cc2c(c(-c3ccnc4ccsc34)c1)OC(c1ocnc1CN1CCC1)C2 ; CNC1CCN(C(=O)C2Cc3cc(Cl)cc(-c4ccnc5ccsc45)c3O2)CC1 ; O=C(C1Cc2cc(Cl)cc(-c3ccnc4ccsc34)c2O1)N1CCC(N2CCCC2)CC1 ; Clc1cc2c(c(-c3ncnc4ccoc34)c1)OC(c1onc3c1CNCC3)C2]

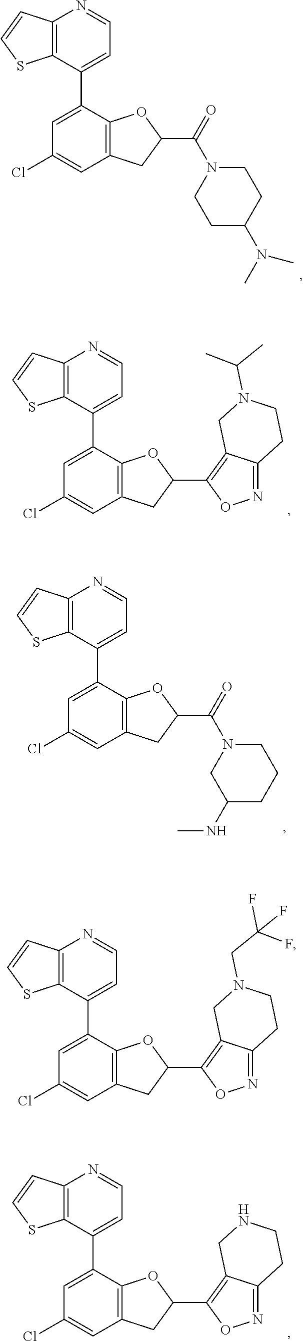 [SMILES: CN(C)C1CCN(C(=O)C2Cc3cc(Cl)cc(-c4ccnc5ccsc45)c3O2)CC1 ; CC(C)N1CCc2noc(C3Cc4cc(Cl)cc(-c5ccnc6ccsc56)c4O3)c2C1 ; CNC1CCCN(C(=O)C2Cc3cc(Cl)cc(-c4ccnc5ccsc45)c3O2)C1 ; FC(F)(F)CN1CCc2noc(C3Cc4cc(Cl)cc(-c5ccnc6ccsc56)c4O3)c2C1 ; Clc1cc2c(c(-c3ccnc4ccsc34)c1)OC(c1onc3c1CNCC3)C2]

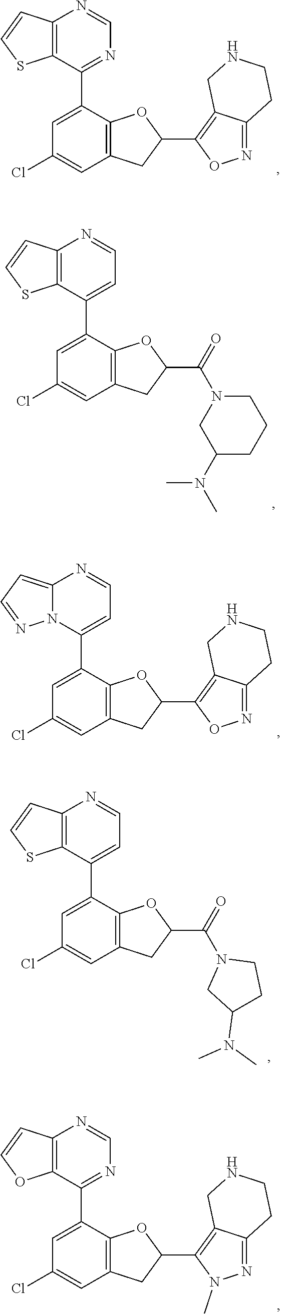 [SMILES: Clc1cc2c(c(-c3ncnc4ccsc34)c1)OC(c1onc3c1CNCC3)C2 ; CN(C)C1CCCN(C(=O)C2Cc3cc(Cl)cc(-c4ccnc5ccsc45)c3O2)C1 ; Clc1cc2c(c(-c3ccnc4ccnn34)c1)OC(c1onc3c1CNCC3)C2 ; CN(C)C1CCN(C(=O)C2Cc3cc(Cl)cc(-c4ccnc5ccsc45)c3O2)C1 ; Cn1nc2c(c1C1Cc3cc(Cl)cc(-c4ncnc5ccoc45)c3O1)CNCC2]

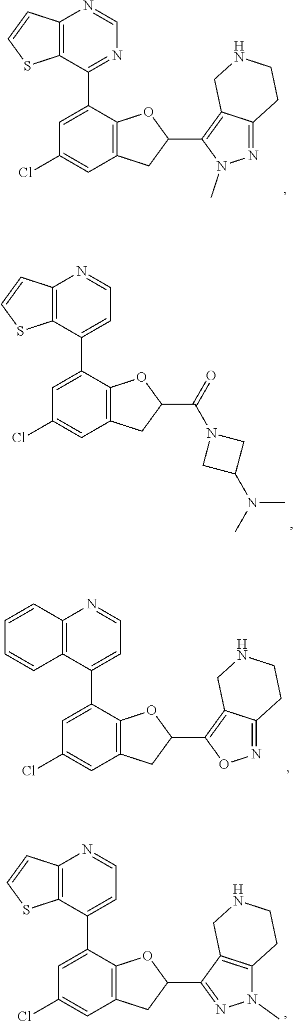 [SMILES: Cn1nc2c(c1C1Cc3cc(Cl)cc(-c4ncnc5ccsc45)c3O1)CNCC2 ; CN(C)C1CN(C(=O)C2Cc3cc(Cl)cc(-c4ccnc5ccsc45)c3O2)C1 ; Clc1cc2c(c(-c3ccnc4ccccc34)c1)OC(c1onc3c1CNCC3)C2 ; Cn1nc(C2Cc3cc(Cl)cc(-c4ccnc5ccsc45)c3O2)c2c1CCNC2]

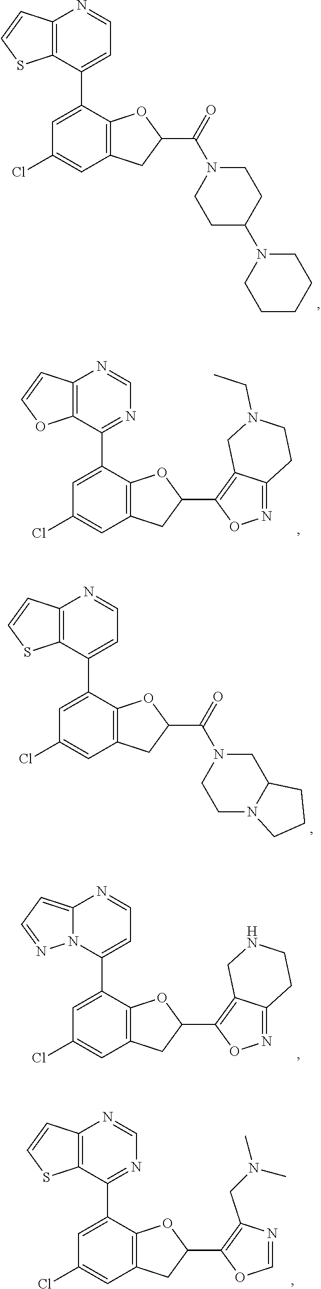 [SMILES: O=C(C1Cc2cc(Cl)cc(-c3ccnc4ccsc34)c2O1)N1CCC(N2CCCCC2)CC1 ; CCN1CCc2noc(C3Cc4cc(Cl)cc(-c5ncnc6ccoc56)c4O3)c2C1 ; O=C(C1Cc2cc(Cl)cc(-c3ccnc4ccsc34)c2O1)N1CCN2CCCC2C1 ; Clc1cc2c(c(-c3ccnc4ccnn34)c1)OC(c1onc3c1CNCC3)C2 ; CN(C)Cc1ncoc1C1Cc2cc(Cl)cc(-c3ncnc4ccsc34)c2O1]

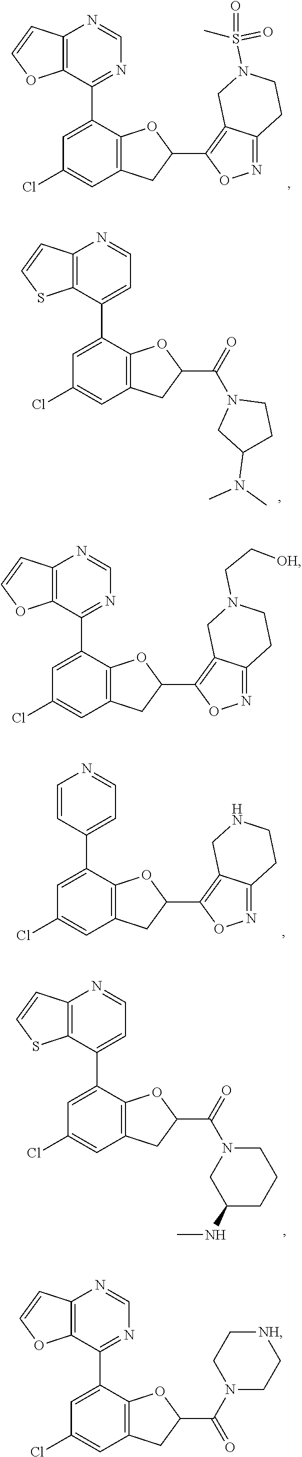

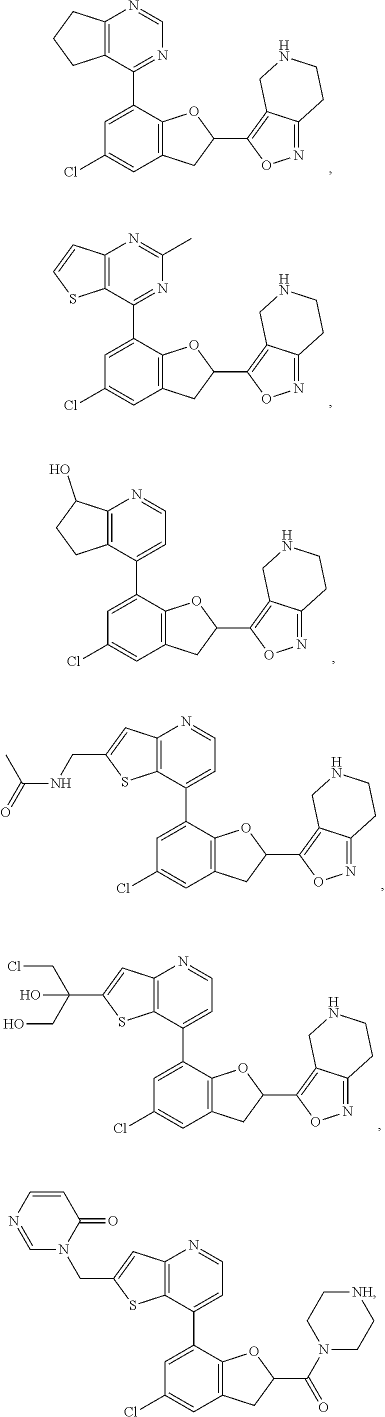 [SMILES: Clc1cc2c(c(-c3ncnc4c3CCC4)c1)OC(c1onc3c1CNCC3)C2 ; Cc1nc(-c2cc(Cl)cc3c2OC(c2onc4c2CNCC4)C3)c2sccc2n1 ; OC1CCc2c(-c3cc(Cl)cc4c3OC(c3onc5c3CNCC5)C4)ccnc21 ; CC(=O)NCc1cc2nccc(-c3cc(Cl)cc4c3OC(c3onc5c3CNCC5)C4)c2s1 ; OCC(O)(CCl)c1cc2nccc(-c3cc(Cl)cc4c3OC(c3onc5c3CNCC5)C4)c2s1 ; O=C(C1Cc2cc(Cl)cc(-c3ccnc4cc(Cn5cnccc5=O)sc34)c2O1)N1CCNCC1]

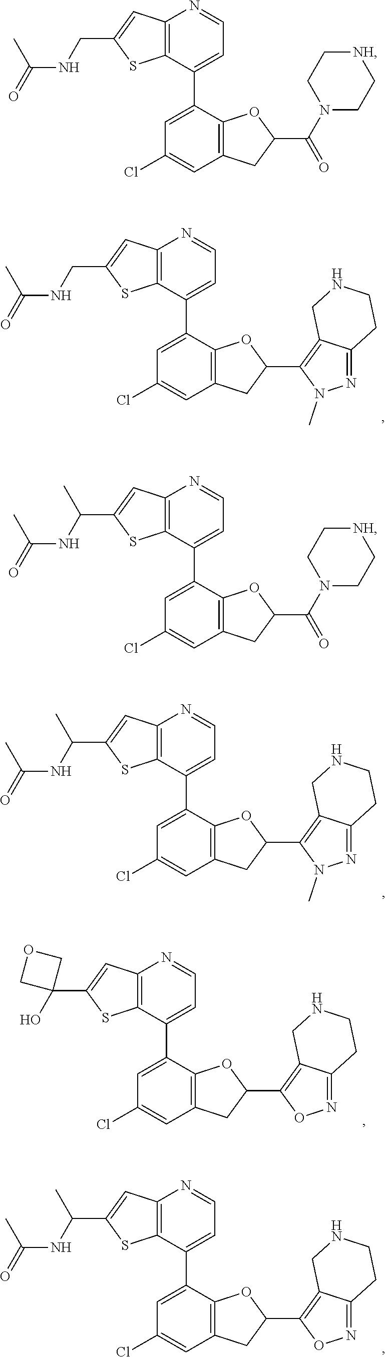 [SMILES: CC(=O)NCc1cc2nccc(-c3cc(Cl)cc4c3OC(C(=O)N3CCNCC3)C4)c2s1 ; CC(=O)NCc1cc2nccc(-c3cc(Cl)cc4c3OC(c3c5c(nn3C)CCNC5)C4)c2s1 ; CC(=O)NC(C)c1cc2nccc(-c3cc(Cl)cc4c3OC(C(=O)N3CCNCC3)C4)c2s1 ; CC(=O)NC(C)c1cc2nccc(-c3cc(Cl)cc4c3OC(c3c5c(nn3C)CCNC5)C4)c2s1 ; OC1(c2cc3nccc(-c4cc(Cl)cc5c4OC(c4onc6c4CNCC6)C5)c3s2)COC1 ; CC(=O)NC(C)c1cc2nccc(-c3cc(Cl)cc4c3OC(c3onc5c3CNCC5)C4)c2s1]

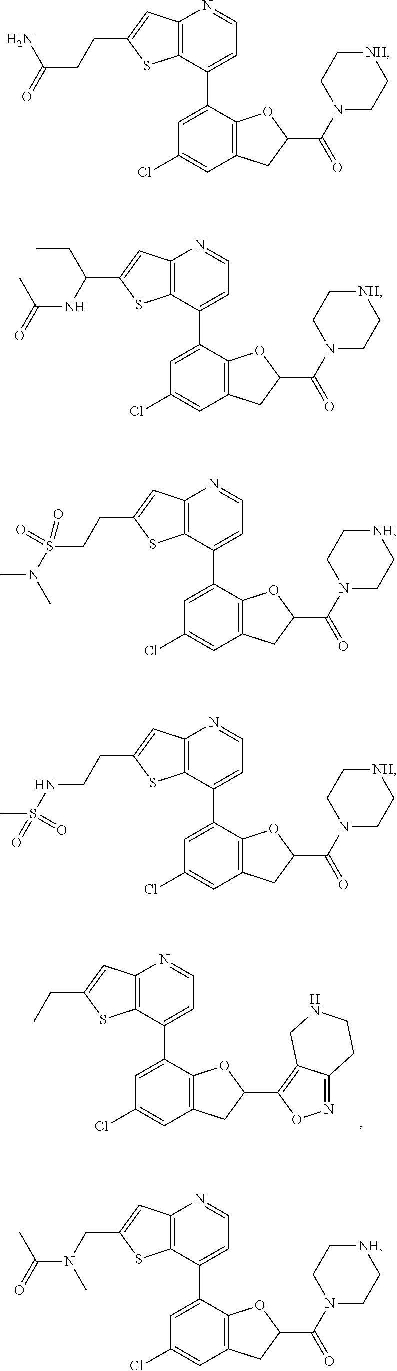 [SMILES: NC(=O)CCc1cc2nccc(-c3cc(Cl)cc4c3OC(C(=O)N3CCNCC3)C4)c2s1 ; CCC(NC(C)=O)c1cc2nccc(-c3cc(Cl)cc4c3OC(C(=O)N3CCNCC3)C4)c2s1 ; CN(C)S(=O)(=O)CCc1cc2nccc(-c3cc(Cl)cc4c3OC(C(=O)N3CCNCC3)C4)c2s1 ; CS(=O)(=O)NCCc1cc2nccc(-c3cc(Cl)cc4c3OC(C(=O)N3CCNCC3)C4)c2s1 ; CCc1cc2nccc(-c3cc(Cl)cc4c3OC(c3onc5c3CNCC5)C4)c2s1 ; CC(=O)N(C)Cc1cc2nccc(-c3cc(Cl)cc4c3OC(C(=O)N3CCNCC3)C4)c2s1]

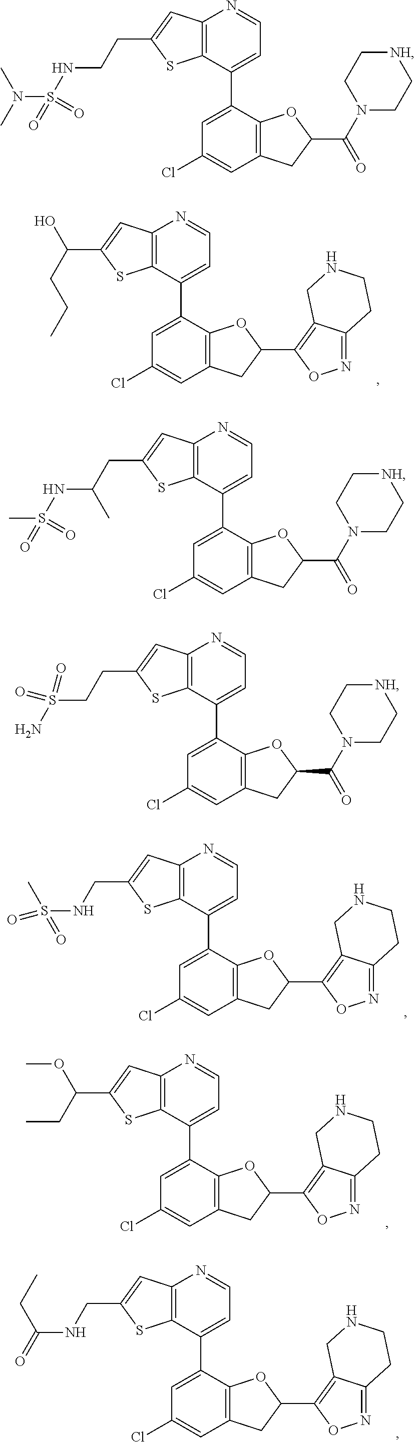

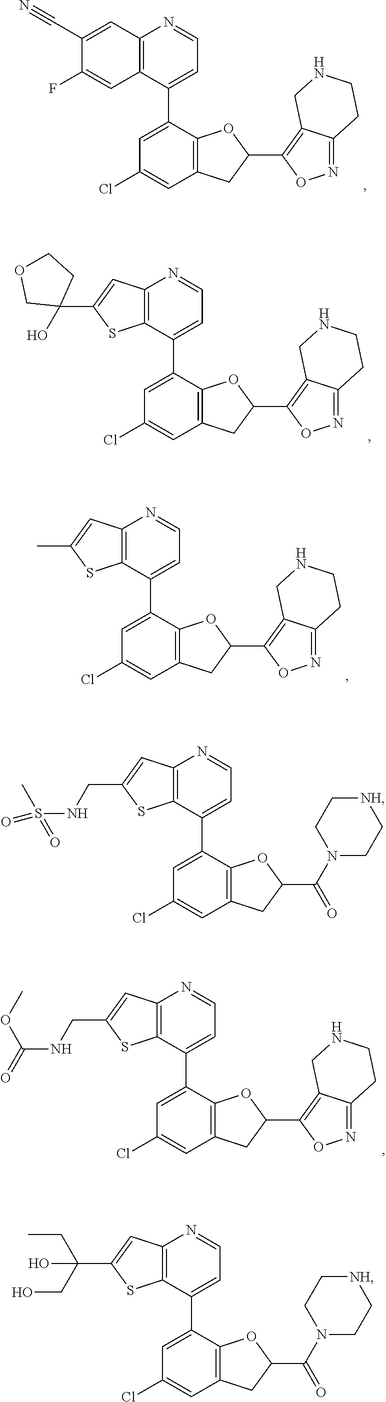 [SMILES: N#Cc1cc2nccc(-c3cc(Cl)cc4c3OC(c3onc5c3CNCC5)C4)c2cc1F ; OC1(c2cc3nccc(-c4cc(Cl)cc5c4OC(c4onc6c4CNCC6)C5)c3s2)CCOC1 ; Cc1cc2nccc(-c3cc(Cl)cc4c3OC(c3onc5c3CNCC5)C4)c2s1 ; CS(=O)(=O)NCc1cc2nccc(-c3cc(Cl)cc4c3OC(C(=O)N3CCNCC3)C4)c2s1 ; COC(=O)NCc1cc2nccc(-c3cc(Cl)cc4c3OC(c3onc5c3CNCC5)C4)c2s1 ; CCC(O)(CO)c1cc2nccc(-c3cc(Cl)cc4c3OC(C(=O)N3CCNCC3)C4)c2s1]

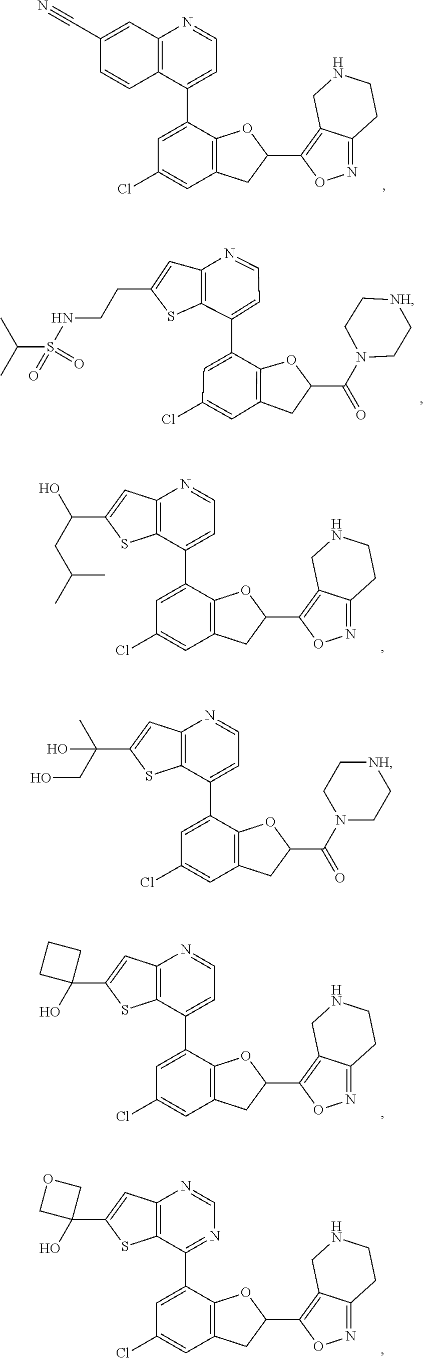 [SMILES: N#Cc1ccc2c(-c3cc(Cl)cc4c3OC(c3onc5c3CNCC5)C4)ccnc2c1 ; CC(C)S(=O)(=O)NCCc1cc2nccc(-c3cc(Cl)cc4c3OC(C(=O)N3CCNCC3)C4)c2s1 ; CC(C)CC(O)c1cc2nccc(-c3cc(Cl)cc4c3OC(c3onc5c3CNCC5)C4)c2s1 ; CC(O)(CO)c1cc2nccc(-c3cc(Cl)cc4c3OC(C(=O)N3CCNCC3)C4)c2s1 ; OC1(c2cc3nccc(-c4cc(Cl)cc5c4OC(c4onc6c4CNCC6)C5)c3s2)CCC1 ; OC1(c2cc3ncnc(-c4cc(Cl)cc5c4OC(c4onc6c4CNCC6)C5)c3s2)COC1]

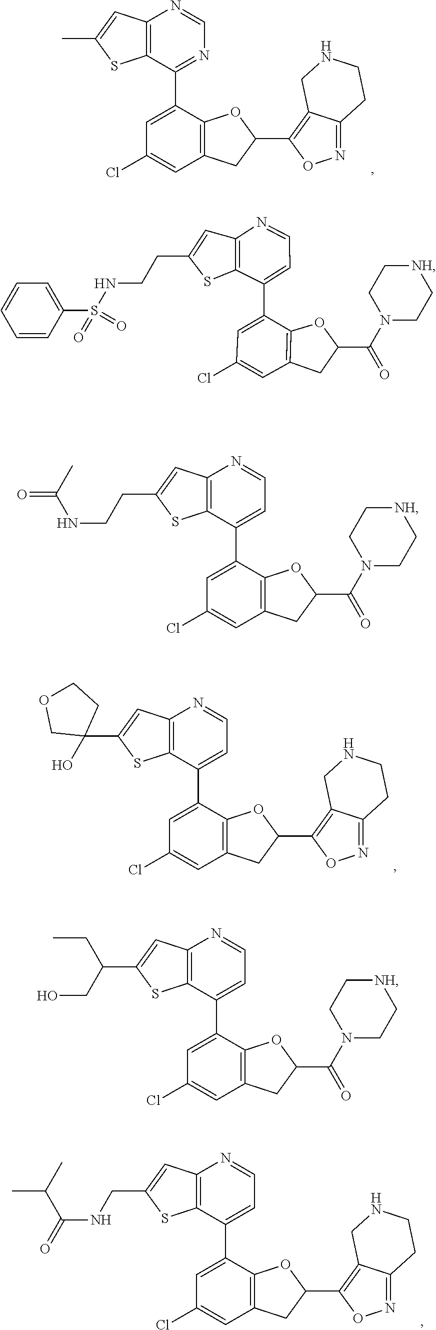 [SMILES: Cc1cc2ncnc(-c3cc(Cl)cc4c3OC(c3onc5c3CNCC5)C4)c2s1 ; O=C(C1Cc2cc(Cl)cc(-c3ccnc4cc(CCNS(=O)(=O)c5ccccc5)sc34)c2O1)N1CCNCC1 ; CC(=O)NCCc1cc2nccc(-c3cc(Cl)cc4c3OC(C(=O)N3CCNCC3)C4)c2s1 ; OC1(c2cc3nccc(-c4cc(Cl)cc5c4OC(c4onc6c4CNCC6)C5)c3s2)CCOC1 ; CCC(CO)c1cc2nccc(-c3cc(Cl)cc4c3OC(C(=O)N3CCNCC3)C4)c2s1 ; CC(C)C(=O)NCc1cc2nccc(-c3cc(Cl)cc4c3OC(c3onc5c3CNCC5)C4)c2s1]

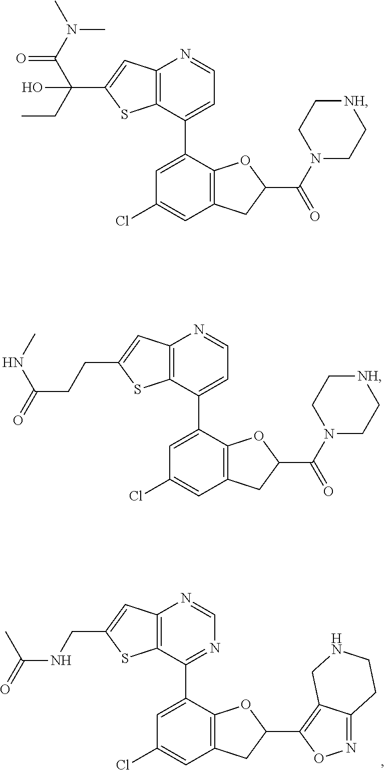 [SMILES: CCC(O)(C(=O)N(C)C)c1cc2nccc(-c3cc(Cl)cc4c3OC(C(=O)N3CCNCC3)C4)c2s1 ; CNC(=O)CCc1cc2nccc(-c3cc(Cl)cc4c3OC(C(=O)N3CCNCC3)C4)c2s1 ; CC(=O)NCc1cc2ncnc(-c3cc(Cl)cc4c3OC(c3onc5c3CNCC5)C4)c2s1]

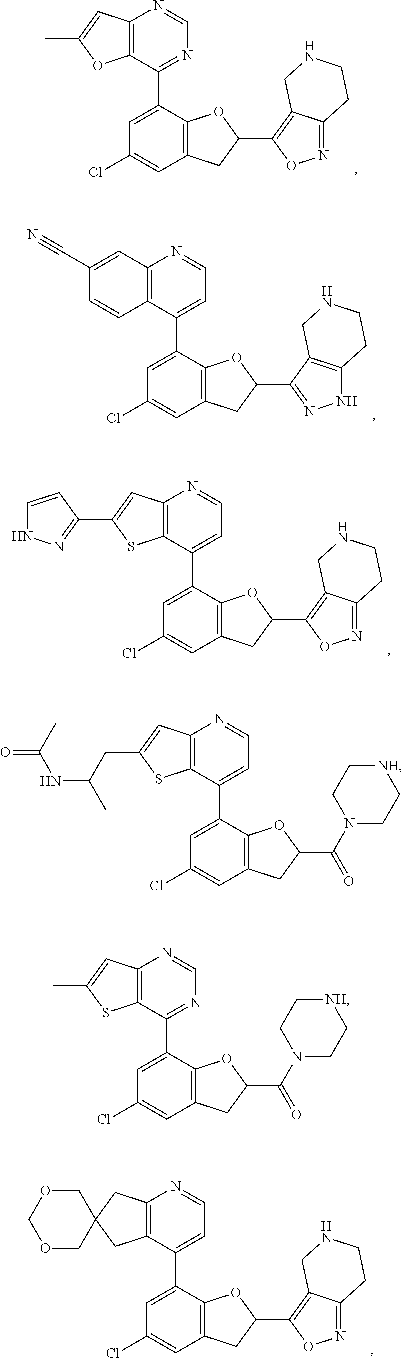 [SMILES: Cc1cc2ncnc(-c3cc(Cl)cc4c3OC(c3onc5c3CNCC5)C4)c2o1 ; N#Cc1ccc2c(-c3cc(Cl)cc4c3OC(c3n[nH]c5c3CNCC5)C4)ccnc2c1 ; Clc1cc2c(c(-c3ccnc4cc(-c5cc[nH]n5)sc34)c1)OC(c1onc3c1CNCC3)C2 ; CC(=O)NC(C)Cc1cc2nccc(-c3cc(Cl)cc4c3OC(C(=O)N3CCNCC3)C4)c2s1 ; Cc1cc2ncnc(-c3cc(Cl)cc4c3OC(C(=O)N3CCNCC3)C4)c2s1 ; Clc1cc2c(c(-c3ccnc4c3CC3(COCOC3)C4)c1)OC(c1onc3c1CNCC3)C2]

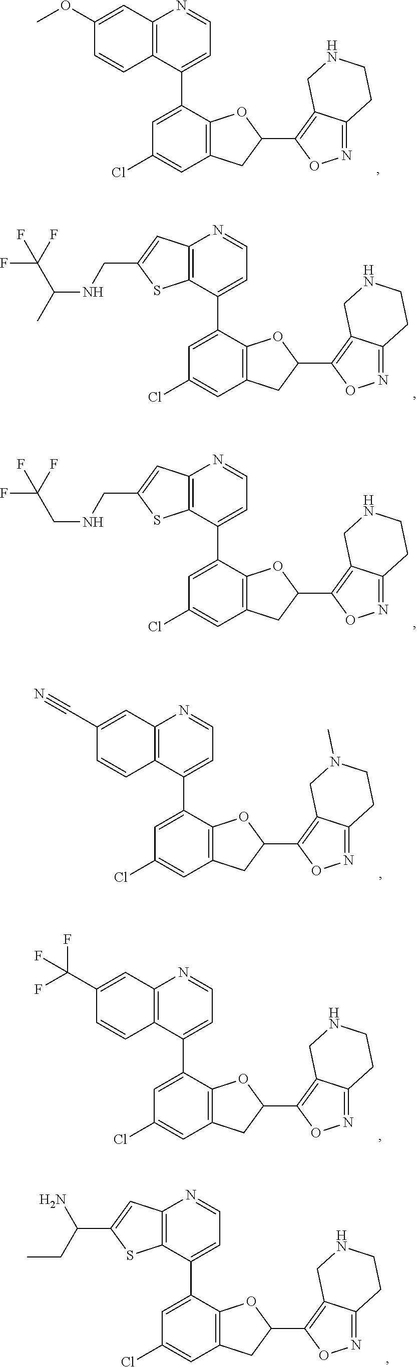 [SMILES: COc1ccc2c(-c3cc(Cl)cc4c3OC(c3onc5c3CNCC5)C4)ccnc2c1 ; CC(NCc1cc2nccc(-c3cc(Cl)cc4c3OC(c3onc5c3CNCC5)C4)c2s1)C(F)(F)F ; FC(F)(F)CNCc1cc2nccc(-c3cc(Cl)cc4c3OC(c3onc5c3CNCC5)C4)c2s1 ; CN1CCc2noc(C3Cc4cc(Cl)cc(-c5ccnc6cc(C#N)ccc56)c4O3)c2C1 ; FC(F)(F)c1ccc2c(-c3cc(Cl)cc4c3OC(c3onc5c3CNCC5)C4)ccnc2c1 ; CCC(N)c1cc2nccc(-c3cc(Cl)cc4c3OC(c3onc5c3CNCC5)C4)c2s1]

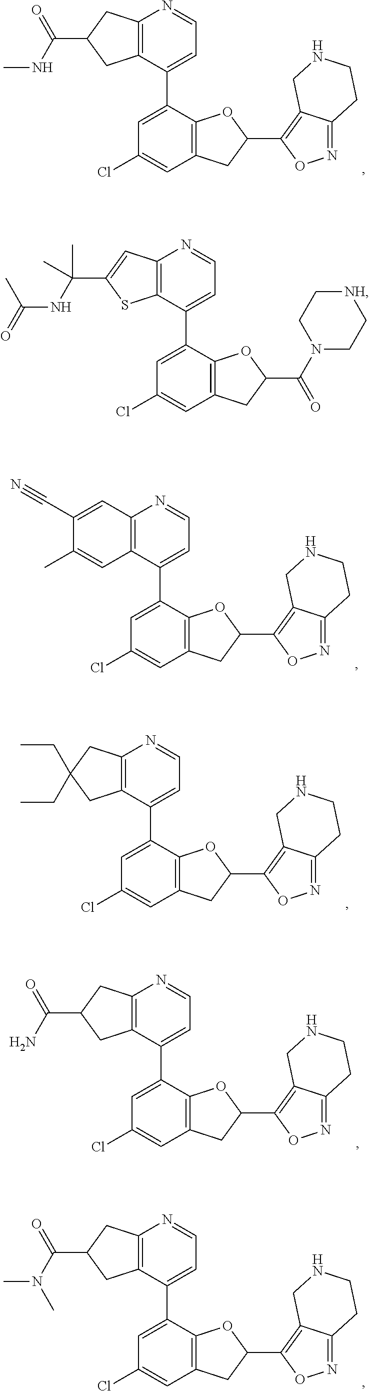 [SMILES: CNC(=O)C1Cc2nccc(-c3cc(Cl)cc4c3OC(c3onc5c3CNCC5)C4)c2C1 ; CC(=O)NC(C)(C)c1cc2nccc(-c3cc(Cl)cc4c3OC(C(=O)N3CCNCC3)C4)c2s1 ; Cc1cc2c(-c3cc(Cl)cc4c3OC(c3onc5c3CNCC5)C4)ccnc2cc1C#N ; CCC1(CC)Cc2nccc(-c3cc(Cl)cc4c3OC(c3onc5c3CNCC5)C4)c2C1 ; NC(=O)C1Cc2nccc(-c3cc(Cl)cc4c3OC(c3onc5c3CNCC5)C4)c2C1 ; CN(C)C(=O)C1Cc2nccc(-c3cc(Cl)cc4c3OC(c3onc5c3CNCC5)C4)c2C1]

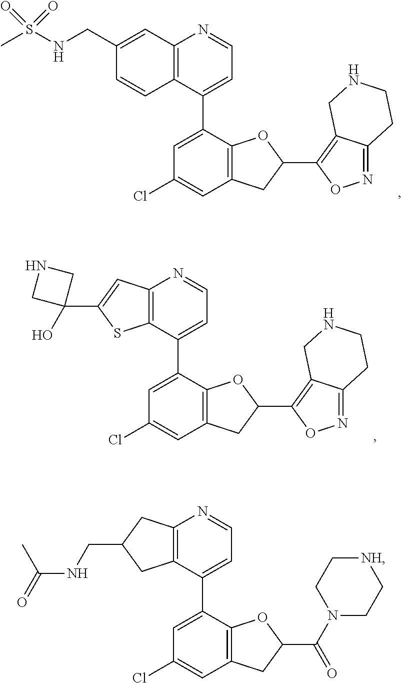 [SMILES: CS(=O)(=O)NCc1ccc2c(-c3cc(Cl)cc4c3OC(c3onc5c3CNCC5)C4)ccnc2c1 ; OC1(c2cc3nccc(-c4cc(Cl)cc5c4OC(c4onc6c4CNCC6)C5)c3s2)CNC1 ; CC(=O)NCC1Cc2nccc(-c3cc(Cl)cc4c3OC(C(=O)N3CCNCC3)C4)c2C1]

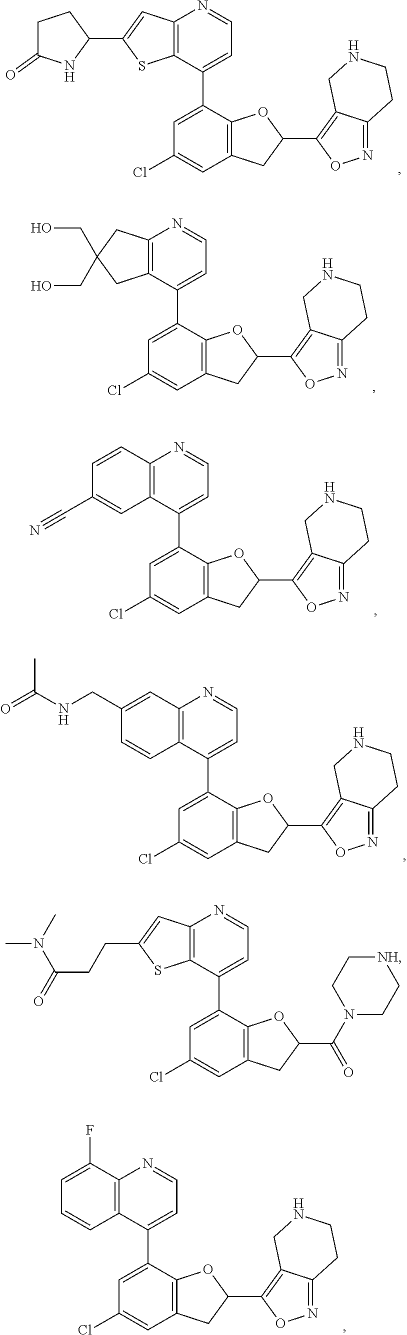 [SMILES: O=C1CCC(c2cc3nccc(-c4cc(Cl)cc5c4OC(c4onc6c4CNCC6)C5)c3s2)N1 ; OCC1(CO)Cc2nccc(-c3cc(Cl)cc4c3OC(c3onc5c3CNCC5)C4)c2C1 ; N#Cc1ccc2nccc(-c3cc(Cl)cc4c3OC(c3onc5c3CNCC5)C4)c2c1 ; CC(=O)NCc1ccc2c(-c3cc(Cl)cc4c3OC(c3onc5c3CNCC5)C4)ccnc2c1 ; CN(C)C(=O)CCc1cc2nccc(-c3cc(Cl)cc4c3OC(C(=O)N3CCNCC3)C4)c2s1 ; Fc1cccc2c(-c3cc(Cl)cc4c3OC(c3onc5c3CNCC5)C4)ccnc12]

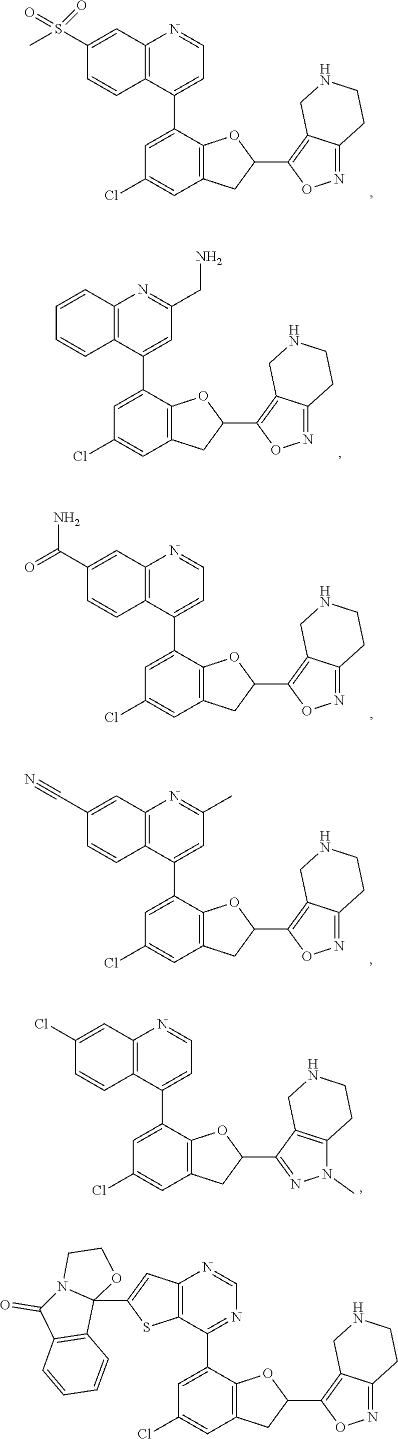 [SMILES: CS(=O)(=O)c1ccc2c(-c3cc(Cl)cc4c3OC(c3onc5c3CNCC5)C4)ccnc2c1 ; NCc1cc(-c2cc(Cl)cc3c2OC(c2onc4c2CNCC4)C3)c2ccccc2n1 ; NC(=O)c1ccc2c(-c3cc(Cl)cc4c3OC(c3onc5c3CNCC5)C4)ccnc2c1 ; Cc1cc(-c2cc(Cl)cc3c2OC(c2onc4c2CNCC4)C3)c2ccc(C#N)cc2n1 ; Cn1nc(C2Cc3cc(Cl)cc(-c4ccnc5cc(Cl)ccc45)c3O2)c2c1CCNC2 ; O=C1c2ccccc2C2(c3cc4ncnc(-c5cc(Cl)cc6c5OC(c5onc7c5CNCC7)C6)c4s3)OCCN12]

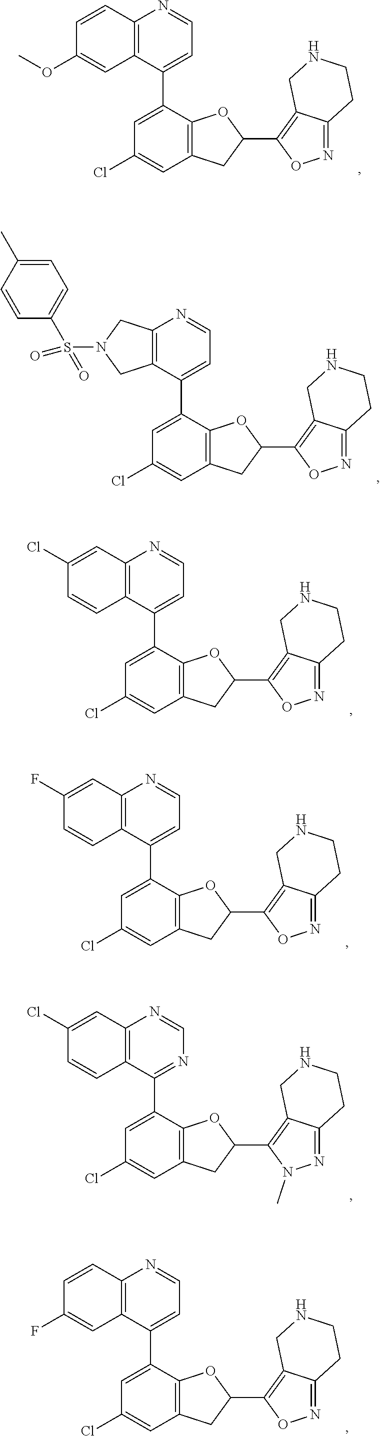 [SMILES: COc1ccc2nccc(-c3cc(Cl)cc4c3OC(c3onc5c3CNCC5)C4)c2c1 ; Cc1ccc(S(=O)(=O)N2Cc3nccc(-c4cc(Cl)cc5c4OC(c4onc6c4CNCC6)C5)c3C2)cc1 ; Clc1cc2c(c(-c3ccnc4cc(Cl)ccc34)c1)OC(c1onc3c1CNCC3)C2 ; Fc1ccc2c(-c3cc(Cl)cc4c3OC(c3onc5c3CNCC5)C4)ccnc2c1 ; Cn1nc2c(c1C1Cc3cc(Cl)cc(-c4ncnc5cc(Cl)ccc45)c3O1)CNCC2 ; Fc1ccc2nccc(-c3cc(Cl)cc4c3OC(c3onc5c3CNCC5)C4)c2c1]

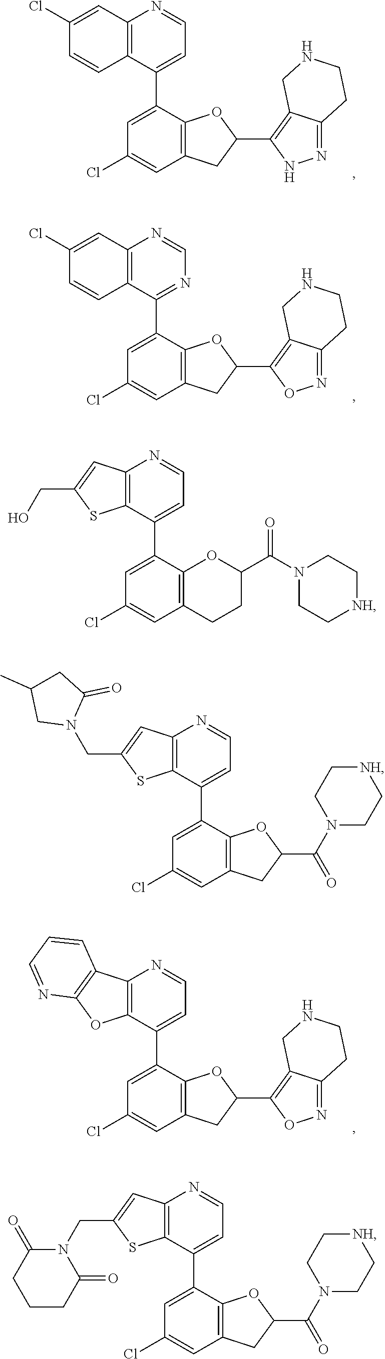 [SMILES: Clc1cc2c(c(-c3ccnc4cc(Cl)ccc34)c1)OC(c1[nH]nc3c1CNCC3)C2 ; Clc1cc2c(c(-c3ncnc4cc(Cl)ccc34)c1)OC(c1onc3c1CNCC3)C2 ; O=C(C1CCc2cc(Cl)cc(-c3ccnc4cc(CO)sc34)c2O1)N1CCNCC1 ; CC1CC(=O)N(Cc2cc3nccc(-c4cc(Cl)cc5c4OC(C(=O)N4CCNCC4)C5)c3s2)C1 ; Clc1cc2c(c(-c3ccnc4c3oc3ncccc34)c1)OC(c1onc3c1CNCC3)C2 ; O=C(C1Cc2cc(Cl)cc(-c3ccnc4cc(CN5C(=O)CCCC5=O)sc34)c2O1)N1CCNCC1]

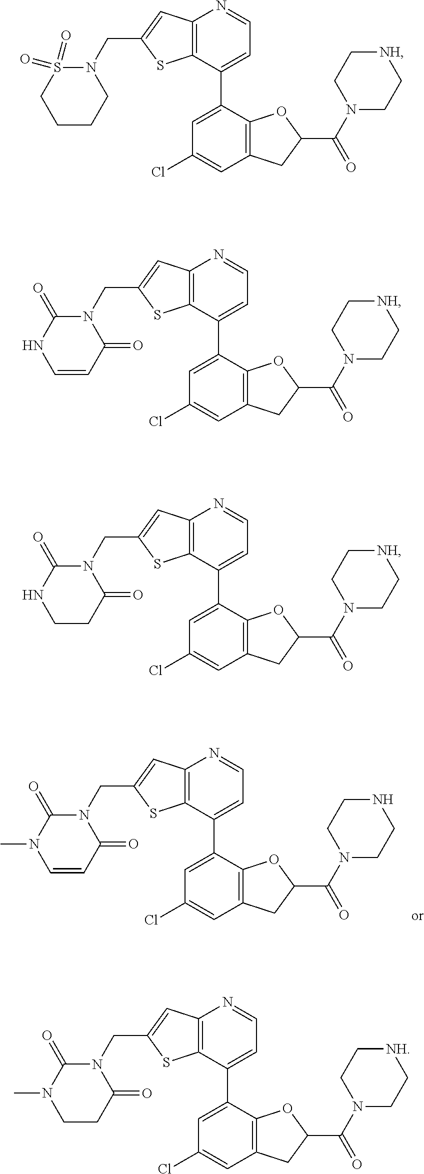 [SMILES: O=C(C1Cc2cc(Cl)cc(-c3ccnc4cc(CN5CCCCS5(=O)=O)sc34)c2O1)N1CCNCC1 ; O=C(C1Cc2cc(Cl)cc(-c3ccnc4cc(Cn5c(=O)cc[nH]c5=O)sc34)c2O1)N1CCNCC1 ; O=C(C1Cc2cc(Cl)cc(-c3ccnc4cc(CN5C(=O)CCNC5=O)sc34)c2O1)N1CCNCC1 ; Cn1ccc(=O)n(Cc2cc3nccc(-c4cc(Cl)cc5c4OC(C(=O)N4CCNCC4)C5)c3s2)c1=O ; CN1CCC(=O)N(Cc2cc3nccc(-c4cc(Cl)cc5c4OC(C(=O)N4CCNCC4)C5)c3s2)C1=O]

### Embodiment P118

A pharmaceutical composition, comprising the compound of any one of embodiments P63 to P117 and a pharmaceutically acceptable excipient.

### Embodiment P119

A method of inhibiting ubiquitin-specific-processing protease 7 (USP7), comprising contacting USP7 with a compound of any one of embodiments P63 to P117 or the pharmaceutical composition of embodiment P118.

### Embodiment P120

A method of treating a USP7-mediated disease or disorder, comprising administering to a patient in need thereof a therapeutically effective amount of a compound of any one of embodiments P63 to P117 or the pharmaceutical composition of embodiment P118.

### Embodiment P121

The method of embodiment P120, wherein the USP7-mediated disease or disorder is an immune disorder, a cardiovascular disease, a viral infection, inflammation, a metabolism/endocrine disorder or a neurological disorder.

### Embodiment P122

The method of embodiment P120, wherein the USP7-mediated disease or disorder is cancer.

### Embodiment P123

The method of embodiment P122, wherein the cancer is breast cancer, ovarian cancer, cervical cancer, prostate cancer, cancer of the testes, genitourinary tract cancer, cancer of the esophagus, cancer of the larynx, glioblastoma, neuroblastoma, stomach cancer, skin cancer, keratoacanthoma, lung cancer, epidermoid carcinoma, large cell carcinoma, non-small cell lung carcinoma (NSCLC), small cell carcinoma, lung adenocarcinoma, bone cancer, colon cancer, adenoma, pancreatic cancer, adenocarcinoma, thyroid cancer, follicular carcinoma, undifferentiated carcinoma, papillary carcinoma, seminoma, melanoma, sarcoma, bladder carcinoma, liver carcinoma and biliary passages, kidney carcinoma, lymphoma, cancer of the pharynx, cancer of the lip, cancer of the tongue, cancer of the mouth, cancer of the small intestine, colorectal cancer, cancer of the large intestine, rectal cancer, brain cancer Hodgkin's, leukemia, cancer of the bronchus, cancer of the thyroid, liver cancer, intrahepatic bile duct cancer, gastric cancer, glioma/glioblastoma, endometrial cancer, melanoma, kidney cancer, renal cancer, pelvic cancer, urinary bladder cancer, multiple myeloma, acute myelogenous leukemia, chronic myelogenous leukemia, lymphocytic leukemia, chronic lymphoid leukemia (CLL), myeloid leukemia, cancer of the oral cavity and pharynx, non-Hodgkin lymphoma, melanoma or villous colon adenoma.

### Embodiment P124

The method of embodiment P122 or P123, further comprising administering to the patient a chemotherapeutic agent in combination with the compound of any one of embodiments P63 to P117 or the pharmaceutical composition of embodiment P118.

### FURTHER EMBODIMENTS

### Embodiment 1

A compound of structural Formula (VII) or a pharmaceutically acceptable salt thereof:

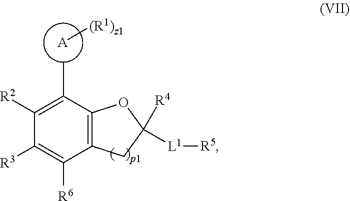

wherein:

-Ring A-(R¹)_{z1} is

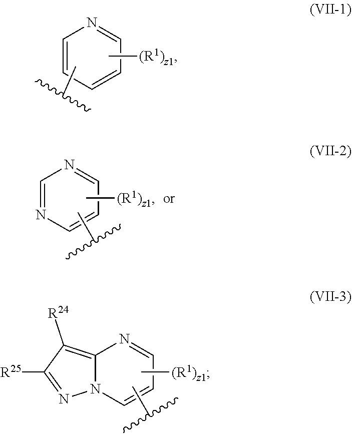

wherein,

p1 is 1 or 2;

L¹ is a bond, —O—, —S—, —NH—, —C(O)—, —C(O)NH, —C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R¹ is independently halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein two R¹ substituents bonded to adjacent carbon atoms may optionally be joined to form a substituted or unsubstituted cycloalkyl, substituted or unsubstituted heterocycloalkyl, substituted or unsubstituted aryl, or substituted or unsubstituted heteroaryl;

R² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₂H, —SOᵥ₂NH₂, —NHNH₂, —ONH₂, —NHC(O)NHNH₂, —NHC(O)NH₂, —N(O)ₘ₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₃H, —SOᵥ₃NH₂, —NHNH₂, —ONH₂, —NHC(O)NHNH₂, —NHC(O)NH₂, —N(O)ₘ₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₄H, —SOᵥ₄NH₂, —NHNH₂, —ONH₂, —NHC(O)NHNH₂, —NHC(O)NH₂, —N(O)ₘ₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁵ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁶ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₆H, —SOᵥ₆NH₂, —NHNH₂, —ONH₂, —NHC(O)NHNH₂, —NHC(O)NH₂, —N(O)ₘ₆, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, | —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₄H, —SOᵥ₂₄NH₂, —NHNH₂, —ONH₂, —NHC(O)NHNH₂, —NHC(O)NH₂, —N(O)ₘ₂₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁵ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₅R^{25A}, —SOᵥ₂₅NR^{25B}R^{25C}, —NHNR^{25B}R^{25C}, —ONR^{25B}R^{25C}, —NHC(O)NHNR^{25B}R^{25C}, —NHC(O)NR^{25B}R^{25C}, —N(O)ₘ₂₅, —NR^{25B}R^{25C}, —C(O)R^{25D}, —C(O)OR^{25D}, —C(O)NR^{25B}R^{25C}, —OR^{25A}, —NR^{25B}SO₂R^{25A}—NR^{25B}C(O)R^{25D}, —R^{25B}C(O)OR^{25D}, —NR^{25B}OR^{25D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R^{1A}, R^{1B}, R^{1C}, R^{1D}, R^{5A}, R^{5B}, R^{5C}, R^{5D}, R^{25A}, R^{25B}, R^{25C}, and R^{25D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C}, R^{5B} and R^{5C}, or R^{25B} and R^{25C} substituents bonded to the same nitrogen atom may optionally be joined to form a substituted or unsubstituted heterocycloalkyl or substituted or unsubstituted heteroaryl;

n1, n2, n3, n4, n5, n6, n24, and n25 are independently an integer from 0 to 4;

m1, m2, m3, m4, m5, m6, m24, m25, v1, v2, v3, v4, v5, v6, v24, and v25 are independently 1 or 2; and

z1 is an integer from 0 to 4.

### Embodiment 2

The compound of embodiment 1, or a pharmaceutically acceptable salt thereof, wherein:

-Ring A-(R¹)_{z1} is:

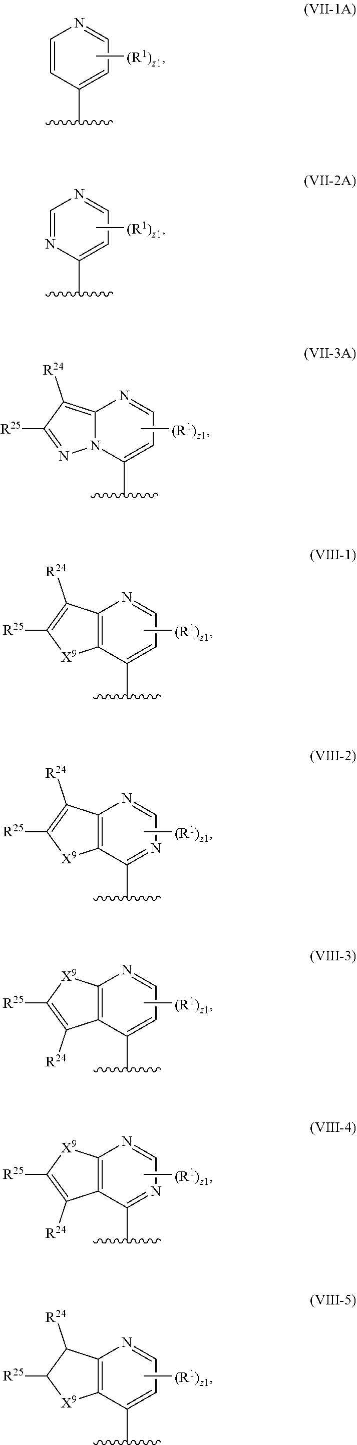

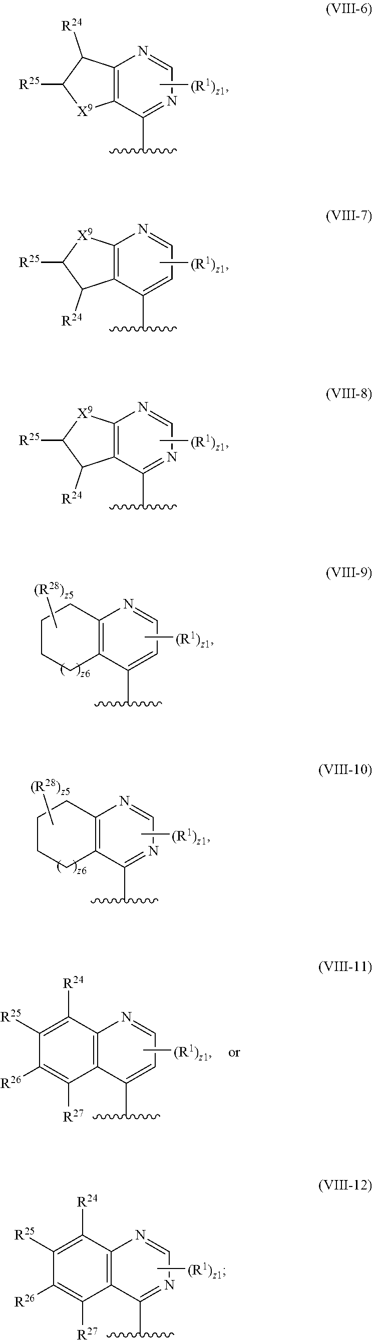

X⁹ is —O—, —S— or —N(R³³)—;

R²⁶ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₆R^{26A}, —SOᵥ₂₆NR^{26B}R^{26C}, —NHNR^{26B}R^{26C}, —ONR^{26B}R^{26C}, —NHC(O)NHNR^{26B}R^{26C}, —NHC(O)NR^{26B}R^{26C}, —N(O)ₘ₂₆, —NR^{26B}R^{26C}, —C(O)R^{26D}, —C(O)OR^{26D}, —C(O)NR^{26B}R^{26C}, —OR^{26A}, —NR^{26B}SO₂R^{26A}, —NR²⁶BC(O)R^{26D}, —NR^{26B}C(O)OR^{26D}, —NR^{26B}OR^{26D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R²⁷ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₇R^{27A}, —SOᵥ₂₇NR^{27B}R^{27C}, —NHNR^{27B}R^{27C}, —ONR^{27B}R^{27C}, —NHC(O)NHNR^{27B}R^{27C}, —NHC(O)NR^{27B}R^{27C}, —N(O)ₘ₂₇, —NR^{27B}R^{27C}, —C(O)R^{27D}, —C(O)OR^{27D}, —C(O)NR^{27B}R^{27C}, —OR^{27A}, —NR^{27B}SO₂R^{27A}, —NR^{27B}C(O)R^{27D}, —NR^{27B}C(O)OR^{27D}, —NR^{27B}OR^{27D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R²⁸ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₈H, —SOᵥ₂₈NH₂, —NHNH₂, —ONH₂, —NHC(O)NHNH₂, —NHC(O)NH₂, —N(O)ₘ₂₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R³³ is independently hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₃₃H, —SOᵥ₃₃NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{26A}, R^{26B}, R^{26C}, R^{26D}, R^{27A}, R^{27B}, R^{27C}, and R^{27D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{26B} and R^{26C}, or R^{27B} and R^{27C} substituents bonded to the same nitrogen atom may optionally be joined to form a substituted or unsubstituted heterocycloalkyl or substituted or unsubstituted heteroaryl;

z5 is an integer from 0 to 8;

z6 is 0 or 1;

n26, n27, n28, and n33 are independently an integer from 0 to 4; and

m26, m27, m28, m33, v26, v27, v28, and v33 are independently 1 or 2.

### Embodiment 3

The compound of embodiment 1 or 2, or a pharmaceutically acceptable salt thereof, wherein the compound has structural Formula (IX):

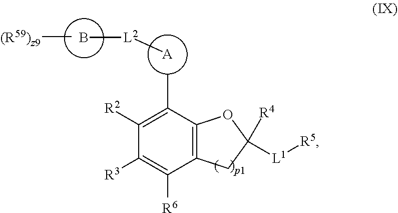

wherein:

Ring B is substituted or unsubstituted cycloalkyl, substituted or unsubstituted heterocycloalkyl, substituted or unsubstituted aryl, or substituted or unsubstituted heteroaryl;

L² is a bond, —O—, —S—, —NH—, —C(O)—, —C(O)NH, —C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and

R⁵⁹ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₅₉H, —SOᵥ₅₉NH₂, —NHNH₂, —ONH₂, —NHC(O)NHNH₂, —NHC(O)NH₂, —N(O)ₘ₅₉, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein two R⁵⁹ substituents bonded to adjacent atoms may optionally be joined to form a substituted or unsubstituted cycloalkyl, substituted or unsubstituted heterocycloalkyl, substituted or unsubstituted aryl, or substituted or unsubstituted heteroaryl;

n59 is an integer from 0 to 4;

m59 and v59 are independently 1 or 2; and

z9 is an integer from 0 to 6.

### Embodiment 4

The compound of any one of embodiments 1-3, or a pharmaceutically acceptable salt thereof, wherein L¹ is a bond or —C(O)—.

### Embodiment 5

The compound of any one of embodiments 1-4, or a pharmaceutically acceptable salt thereof, wherein R⁵ is substituted or unsubstituted cycloalkyl, substituted or unsubstituted heterocycloalkyl, substituted or unsubstituted aryl, or substituted or unsubstituted heteroaryl.

### Embodiment 6

The compound of any one of embodiments 1-5, or a pharmaceutically acceptable salt thereof, wherein:

L¹ is a bond; and

R⁵ is a substituted or unsubstituted heterocycloalkyl or a substituted or unsubstituted heteroaryl.

### Embodiment 7

The compound of any one of embodiments 1-5, or a pharmaceutically acceptable salt thereof, wherein R⁵ is a substituted or unsubstituted 5,6-fused ring system, wherein the 5-membered ring of the 5,6-fused ring system is covalently bound to L¹.

### Embodiment 8

The compound of embodiment 1 or 2, or a pharmaceutically acceptable salt thereof, wherein the compound has structural Formula (X):

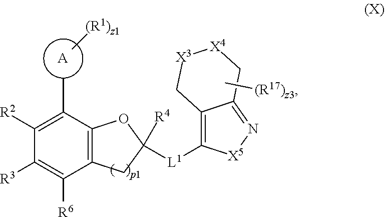

wherein:

X³ is

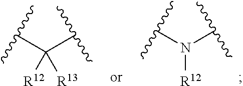

X⁴ is

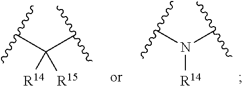

X⁵ is —O—

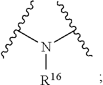

R¹² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —N₃, —CN, —SO₂CH₃, —SOₙ₁₂H, —SOᵥ₁₂NH₂, —NHNH₂, —ONH₂, —NHC(O)NHNH₂, —NHC(O)NH₂, —N(O)ₘ₁₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹³ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₃H, —SOᵥ₁₃NH₂, —NHNH₂, —ONH₂, —NHC(O)NHNH₂, —NHC(O)NH₂, —N(O)ₘ₁₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹² and R¹³ are optionally together oxo;

R¹⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₄H, —SOᵥ₁₄NH₂, —NHNH₂, —ONH₂, —NHC(O)NHNH₂, —NHC(O)NH₂, —N(O)ₘ₁₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹⁵ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₅H, —SOᵥ₁₅NH₂, —NHNH₂, —ONH₂, —NHC(O)NHNH₂, —NHC(O)NH₂, —N(O)ₘ₁₅, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹⁴ and R¹⁵ are optionally together oxo;

R¹⁶ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₆H, —SOᵥ₁₆NH₂, —NHNH₂, —ONH₂, —NHC(O)NHNH₂, —NHC(O)NH₂, —N(O)ₘ₁₆, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

R¹⁷ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₁₇H, —SOᵥ₁₇NH₂, —NHNH₂, —ONH₂, —NHC(O)NHNH₂, —NHC(O)NH₂, —N(O)ₘ₁₇, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

n12, n13, n14, n15, n16 and n17 are independently an integer from 0 to 4;

m12, m13, m14, m15, m16, m17, v12, v13, v14, v15, v16 and v17 are independently 1 or 2; and

z3 is an integer from 0 to 4.

### Embodiment 9

The compound of embodiment 8, or a pharmaceutically acceptable salt thereof, wherein z3 is 0.

### Embodiment 10

The compound of embodiment 8 or 9, or a pharmaceutically acceptable salt thereof, wherein:

X³ is

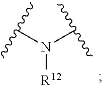

X⁴ is

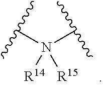

### Embodiment 11

The compound of any one of embodiments 8-10, or a pharmaceutically acceptable salt thereof, wherein R¹⁴ and R¹⁵ are hydrogen.

### Embodiment 12

The compound of any one of embodiments 8-11, or a pharmaceutically acceptable salt thereof, wherein R¹² is hydrogen.

### Embodiment 13

The compound of embodiment 8 or 9, or a pharmaceutically acceptable salt thereof, wherein:

X³ is

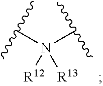

X⁴ is

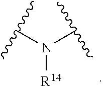

### Embodiment 14

The compound of any one of embodiments 8-13, or a pharmaceutically acceptable salt thereof, wherein R¹² and R¹³ are hydrogen.

### Embodiment 15

The compound of any one of Embodiments 8-14, or a pharmaceutically acceptable salt thereof, wherein R¹⁴ is hydrogen.

### Embodiment 16

The compound of any one of embodiments 8-15, or a pharmaceutically acceptable salt thereof, wherein X⁵ is —O—.

### Embodiment 17

The compound of any one of embodiments 8-15, or a pharmaceutically acceptable salt thereof, wherein X⁵ is

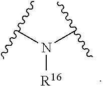

### Embodiment 18

The compound of embodiment 1 or 2, or a pharmaceutically acceptable salt thereof, wherein the compound has structural Formula (XI):

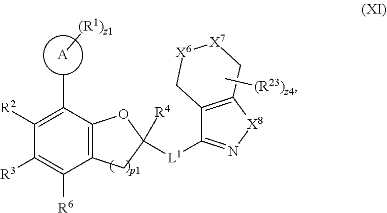

wherein:

X⁶ is

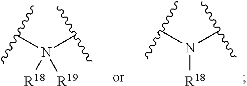

X⁷ is

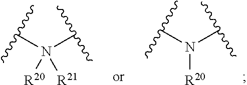

X⁸ is —O— or

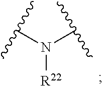

R¹⁸ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₈H, —SOᵥ₁₈NH₂, —NHNH₂, —ONH₂, —NHC(O)NHNH₂, —NHC(O)NH₂, —N(O)ₘ₁₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹⁹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₉H, —SOᵥ₁₉NH₂, —NHNH₂, —ONH₂, —NHC(O)NHNH₂, —NHC(O)NH₂, —N(O)ₘ₁₉, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁸ and R¹⁹ are optionally together oxo;

R²⁰ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₂₀H, —SOᵥ₂₀NH₂, —NHNH₂, —ONH₂, —NHC(O)NHNH₂, —NHC(O)NH₂, —N(O)ₘ₂₀, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²¹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₂₁H, —SOᵥ₂₁NH₂, —NHNH₂, —ONH₂, —NHC(O)NHNH₂, —NHC(O)NH₂, —N(O)ₘ₂₁, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁰ and R²¹ are optionally together oxo;

R²² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₂H, —SO₂CH₃, —SOᵥ₂₂NH₂, —NHNH₂, —ONH₂, —NHC(O)NHNH₂, —NHC(O)NH₂, —N(O)ₘ₂₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

R²³ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₂₃H, —SOᵥ₂₃NH₂, —NHNH₂, —ONH₂, —NHC(O)NHNH₂, —NHC(O)NH₂, —N(O)ₘ₂₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

n18, n19, n20, n21, n22 and n23 are independently an integer from 0 to 4;

m18, m19, m20, m21, m22, m23, v18, v19, v20, v21, v22 and v23 are independently 1 or 2; and

z4 is an integer from 0 to 4.

### Embodiment 19

The compound of embodiment 18, or a pharmaceutically acceptable salt thereof, wherein z4 is 0.

### Embodiment 20

The compound of embodiment 18 or 19, or a pharmaceutically acceptable salt thereof, wherein X⁶ is

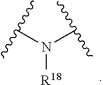

### Embodiment 21

The compound of any one of embodiments 18-20, or a pharmaceutically acceptable salt thereof, wherein X⁷ is

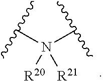

### Embodiment 22

The compound of any one of embodiments 18-21, or a pharmaceutically acceptable salt thereof, wherein R²⁰ is hydrogen.

### Embodiment 23

The compound of any one of embodiments 18-22, or a pharmaceutically acceptable salt thereof, wherein R²¹ is hydrogen.

### Embodiment 24

The compound of any one of embodiments 18-23, or a pharmaceutically acceptable salt thereof, wherein R¹⁸ is hydrogen.

### Embodiment 25

The compound of any one of embodiments 18-24, or a pharmaceutically acceptable salt thereof, wherein X⁸ is —O—.

### Embodiment 26

The compound of any one of embodiments 18-25, or a pharmaceutically acceptable salt thereof, wherein X⁸ is

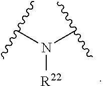

### Embodiment 27

The compound of any one of embodiments 1-7, or a pharmaceutically acceptable salt thereof, wherein:

R⁵ is

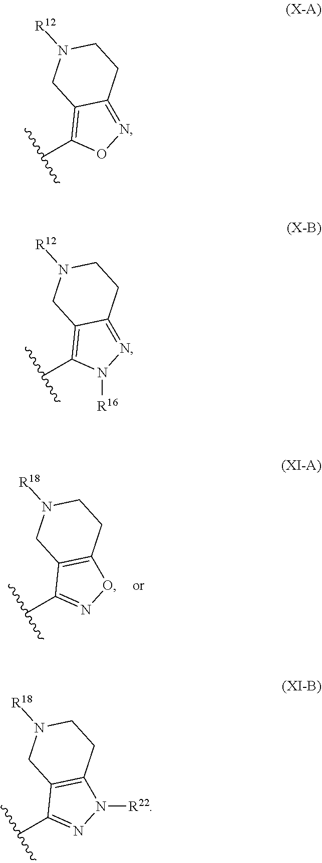

### Embodiment 28

The compound of any one of embodiment 1, 2, or 8, or a pharmaceutically acceptable salt thereof, wherein the compound has structural Formula (X-1A):

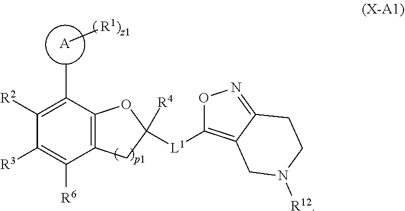

### Embodiment 29

The compound of embodiment 1 or 2, or a pharmaceutically acceptable salt thereof, wherein the compound has structural Formula (XII):

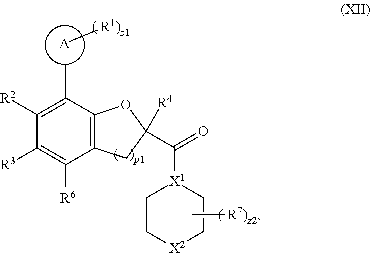

wherein:

X¹ is

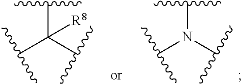

X² is

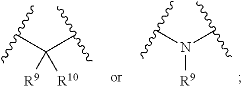

n7, n8, n9 and n10 are independently an integer from 0 to 4;

m7, m8, m9, m10, v7, v8, v9 and v10 are independently 1 or 2; z2 is an integer from 0 to 8;

R⁷ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₇H, —SOᵥ7NH₂, —NHNH₂, —ONH₂, —NHC(O)NHNH₂, —NHC(O)NH₂, —N(O)ₘ₇, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁸ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₈H, —SOᵥ₈NH₂, —NHNH₂, —ONH₂, —NHC(O)NHNH₂, —NHC(O)NH₂, —N(O)ₘ₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₉R^{9A}, —SOᵥ₉NR^{9B}R^{9C}, —NHNR^{9B}R^{9C}, —ONR^{9B}R^{9C}, —NHC(O)NHNR^{9B}R^{9C}, —NHC(O)NR^{9B}R^{9C}, —N(O)ₘ₉, —NR^{9B}R^{9C}, —C(O)R^{9D}, —C(O)OR^{9D}, —C(O)NR^{9B}R^{9C}, —OR^{9A}, —NR^{9B}SO₂R^{9A}, —NR^{9B}C(O)R^{9D}, —NR^{9B}C(O)OR^{9D}, —NR^{9B}OR^{9D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹⁰ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₁₀R^{10A}, —SOᵥ₁₀NR^{10B}R^{10C}, —NHNR^{10B}R^{10C}, —ONR^{10B}R^{10C}, —NHC(O)NHNR^{10B}R^{10C}, —NHC(O)NR^{10B}R^{10C}, —N(O)ₘ₁₀, —NR^{10B}R^{10C}, —C(O)R^{10D}, —C(O)OR^{10D}, —C(O)NR^{10B}R^{10C}, —OR^{10A}, —NR^{10B}SO₂R^{10A}, —NR^{10B}C(O)R^{10D}, —NR^{10B}C(O)OR^{10D}, —NR^{10B}OR^{10D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁹ and R¹⁰ are optionally together oxo; and

R^{9A}, R^{9B}, R^{9C}, R^{9D}, R^{10A}, R^{10B}, R^{10C} and R^{10D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{9B} and R^{9C} or R^{10B} and R^{10C} substituents bonded to the same nitrogen atom may optionally be joined to form a substituted or unsubstituted heterocycloalkyl or substituted or unsubstituted heteroaryl.

### Embodiment 30

The compound of embodiment 29, or a pharmaceutically acceptable salt thereof, wherein X¹ is

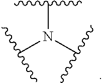 [SMILES: *N(*)*]

### Embodiment 31

The compound of embodiment 29, or a pharmaceutically acceptable salt thereof, wherein X¹ is

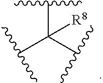

### Embodiment 32

The compound of any one of embodiments 29-31, or a pharmaceutically acceptable salt thereof, wherein X² is

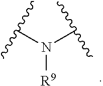

### Embodiment 33

The compound of any one of embodiments 29-31, or a pharmaceutically acceptable salt thereof, wherein X² is

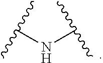 [SMILES: *N*]

### Embodiment 34

The compound of any one of embodiments 29-31, or a pharmaceutically acceptable salt thereof, wherein X² is

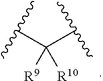

### Embodiment 35

The compound of any one of embodiments 29-34, or a pharmaceutically acceptable salt thereof, wherein R⁹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 36

The compound of any one of embodiments 29-34, or a pharmaceutically acceptable salt thereof, wherein R⁹ is substituted or unsubstituted cycloalkyl, substituted or unsubstituted heterocycloalkyl, substituted or unsubstituted aryl, or substituted or unsubstituted heteroaryl.

### Embodiment 37

The compound of any one of embodiments 29-34, or a pharmaceutically acceptable salt thereof, wherein R⁹ is substituted or unsubstituted pyridinyl.

### Embodiment 38

The compound of any one of embodiments 3-37 or a pharmaceutically acceptable salt thereof, wherein Ring B is substituted or unsubstituted heterocycloalkyl, substituted or unsubstituted aryl, or substituted or unsubstituted heteroaryl.

### Embodiment 39

The compound of any one of embodiments 3-37, or a pharmaceutically acceptable salt thereof, wherein Ring B is a substituted or unsubstituted monovalent succinimide moiety, substituted or unsubstituted monovalent hydantoin moiety, substituted or unsubstituted monovalent oxohydantoin moiety, substituted or unsubstituted monovalent thiohydantoin moiety, substituted or unsubstituted monovalent pyrrolidone moiety, substituted or unsubstituted monovalent phthalimide moiety, substituted or unsubstituted monovalent oxazolidinone moiety, or substituted or unsubstituted monovalent cyclic urea moiety.

### Embodiment 40

The compound of any one of embodiments 3-37, or a pharmaceutically acceptable salt thereof, wherein:

Ring B—(R⁵⁹)z9 is

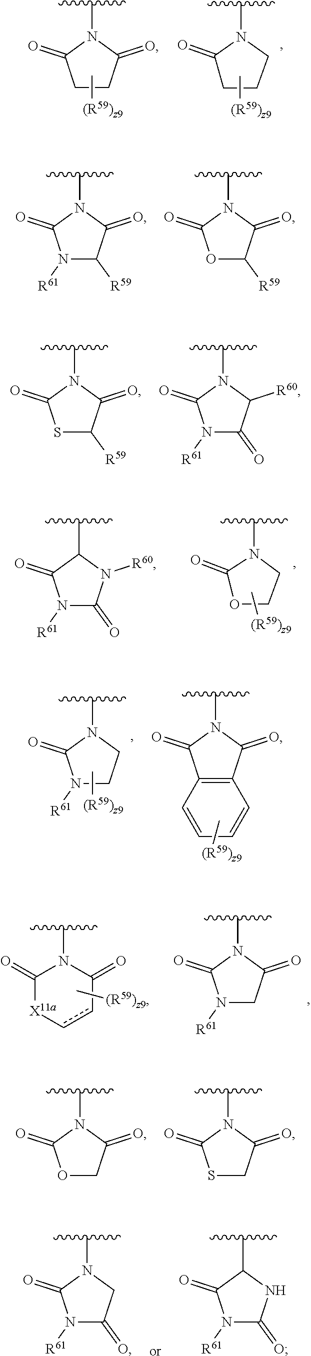

wherein:

X¹¹ is

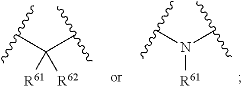

[symbol] is a single bond or double bond;

R⁶⁰ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₆₀R^{60A}, —SOᵥ₆₀NR^{60B}R^{60C}, —NHNR^{60B}R^{60C}, —ONR^{60B}R^{60C}, —NHC(O)NHNR^{60B}R^{60C}, —NHC(O)NR^{60B}R^{60C}, —N(O)ₘ₆₀, —NR^{60B}R^{60C}, —C(O)R^{60D}, —C(O)OR^{60D}, —C(O)NR^{60B}R^{60C}, —OR^{60A}, —NR^{6B}SO₂R^{60A}, —NR^{60B}C(O)R^{60D}, —NR^{6B}C(O)OR^{60D}, —NR^{6B}OR^{60D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁶¹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₆₁R^{61A}, —SO^{61B}R^{61C}, —NHNR^{61B}R^{61C}, —ONR^{61B}R^{61C}, —NHC(O)NHNR^{61B}R^{61C}, —NHC(O)NR^{61B}R^{61C}, —N(O)ₘ₆₁, —NR^{61B}R^{61C}, —C(O)R^{61D}, —C(O)OR^{61D}, —C(O)NR^{61B}R^{61C}, —OR^{61A}, —NR^{61B}SO₂R^{61C}, —NR^{61B}C(O)R^{61D}, —NR^{61B}C(O)OR^{61D}, —NR^{61B}OR^{61D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁶² is independently hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₆₂H, —SOᵥ₆₂NH₂, —NHNH₂, —ONH₂, —NHC(O)NHNH₂, —NHC(O)NH₂, —N(O)ₘ₆₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁶¹ and R⁶² are optionally together oxo; and

R^{61A}, R^{61B}, R^{61C} and R^{61D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{61B} and R^{61C} substituents bonded to the same nitrogen atom may optionally be joined to form a substituted or unsubstituted heterocycloalkyl or substituted or unsubstituted heteroaryl; n60, n61 and n62 are independently an integer from 0 to 4; and m60, m61, m62, v60, v61 and v62 are independently 1 or 2.

### Embodiment 41

The compound of any one of Embodiment 2, or a pharmaceutically acceptable salt thereof, wherein R²⁴ and R²⁵ are independently —CN, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 42

The compound of any one of embodiments 2-40, or a pharmaceutically acceptable salt thereof, wherein R²⁴ and R²⁵ are independently —CH₂OH, —C(O)NH₂, —C(O)NHCH₃, —CH(OH)CH₂CH₃ or —CH(OH)CH₃.

### Embodiment 43

The compound of any one of embodiments 1-42, or a pharmaceutically acceptable salt thereof, wherein R¹ is halogen, —CN, substituted or unsubstituted alkyl or substituted or unsubstituted heteroalkyl.

### Embodiment 44

The compound of any one of embodiments 1-43, or a pharmaceutically acceptable salt thereof, wherein R⁴ is hydrogen, substituted or unsubstituted alkyl or substituted or unsubstituted heteroalkyl.

### Embodiment 45

The compound of any one of embodiments 1-44, or a pharmaceutically acceptable salt thereof, wherein R⁴ is hydrogen or unsubstituted methyl.

### Embodiment 46

The compound of any one of embodiments 1-45, or a pharmaceutically acceptable salt thereof, wherein R² and R³ are independently hydrogen, halogen, substituted or unsubstituted alkyl or substituted or unsubstituted heteroalkyl.

### Embodiment 47

The compound of any one of embodiments 1-46, or a pharmaceutically acceptable salt thereof, wherein R² and R³ are unsubstituted methyl.

### Embodiment 48

The compound of any one of embodiments 1-46, or a pharmaceutically acceptable salt thereof, wherein R² is hydrogen.

### Embodiment 49

The compound of any one of embodiments 1-46, or a pharmaceutically acceptable salt thereof, wherein R³ is halogen, unsubstituted methyl or —CF₃.

### Embodiment 50

The compound of embodiment 8 or 29, or a pharmaceutically acceptable salt thereof, wherein R¹² is —N(CH₃)₂ or —NHCH₃.

### Embodiment 51

The compound of embodiment 8 or 29, or a pharmaceutically acceptable salt thereof, wherein R¹² is —N(CH₃)₂.

### Embodiment 52

The compound of any one of embodiments 1-51, or a pharmaceutically acceptable salt thereof, wherein X⁹ is —S— or —O—.

### Embodiment 53

The compound of any one of embodiments 1-52, or a pharmaceutically acceptable salt thereof, wherein p1 is 1.

### Embodiment 54

The compound of embodiment 1, wherein the compound is of structural Formula (I):

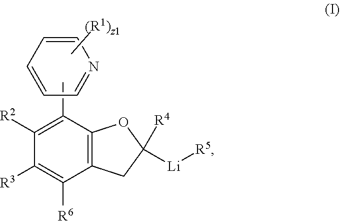

or a pharmaceutically acceptable salt thereof.

### Embodiment 55

The compound of embodiment 1, wherein the compound is of structural Formula (II):

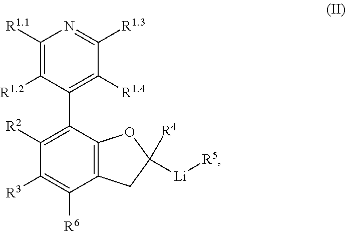

or a pharmaceutically acceptable salt thereof, wherein:

n1.1, n1.2, n1.3 and n1.4 are independently and integer from 0 to 4;

m1.1, m1.2, m1.3, m1.4, v1.1, v1.2, v1.3 and v1.4 are independently 1 or 2;

R^{1.1} is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO_{n1.1}R^{1.1A}, —SO_{v1.1}NR^{1.1B}R^{1.1C}, —NHNR^{1.1B}R^{1.1C}, —ONR^{1.1B}R^{1.1C}, —NHC(O)NHNR^{1.1B}R^{1.1C}, —NHC(O)NR^{1.1B}R^{1.1C}, —N(O)_{m1.1}, —NR^{1.1B}R^{1.1C}, —C(O)R^{1.1D}, —C(O)OR^{1.1D}, —C(O)NR^{1.1B}R^{1.1C}, —OR^{1.1A}, —NR^{1.1B}SO₂R^{1.1A}, —NR^{1.1B}C(O)R^{1.1D}, —NR^{1.1B}C(O)OR^{1.1D}, —NR^{1.1B}OR^{1.1D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R^{1.2} is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO_{n1.2}H, —SO_{v1.2}NH₂, —NHNH₂, —ONH₂, —NHC(O)NHNH₂, —NHC(O)NH₂, —N(O)_{m1.2}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1.3} is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO_{n1.3}H, —SO_{v1.3}NH₂, —NHNH₂, —ONH₂, —NHC(O)NHNH₂, —NHC(O)NH₂, —N(O)_{m1.3}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹⁴ is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO_{n1.4}H, —SO_{v1.4}NH₂, —NHNH₂, —ONH₂, —NHC(O)NHNH₂, —NHC(O)NH₂, —N(O)_{m1.4}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

R^{1.1A}, R^{1.1B}, R^{1.1C} and R^{1.1D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1.1B} and R^{1.1C} substituents bonded to the same nitrogen atom may optionally be joined to form a substituted or unsubstituted heterocycloalkyl or substituted or unsubstituted heteroaryl; and wherein R^{1.1} and R^{1.2} or R^{1.3} and R^{1.4} may optionally be joined to form a substituted or unsubstituted cycloalkyl, substituted or unsubstituted heterocycloalkyl, substituted or unsubstituted aryl or substituted or unsubstituted heteroaryl, and further wherein at least one of R^{1.1}, R^{1.2}, R^{1.3} and R^{1.4} is not hydrogen.

### Embodiment 56

The compound of embodiment 1, wherein the compound is of structural Formula (III):

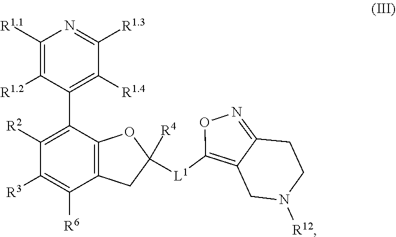

or a pharmaceutically acceptable salt thereof, wherein:

n1.1, n1.2, n1.3, n1.4 and n12 are independently and integer from 0 to 4;

m1.1, m1.2, m1.3, m1.4, m12, v1.1, v1.2, v1.3, v1.4 and v12 are independently 1 or 2;

R^{1.1} is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO_{n1.1}R^{1.1A}, —SO_{v1.1}NR^{1.1B}R^{1.1C}, —NHNR^{1.1B}R^{1.1C}, —ONR^{1.1B}R^{1.1C}, —NHC(O)NHNR^{1.1B}R^{1.1C}, —NHC(O)NR^{1.1B}R^{1.1C}, —N(O)_{m1.1}, —NR^{1.1B}R^{1.1C}, —C(O)R^{1.1D}, —C(O)OR^{1.1D}, —C(O)NR^{1.1B}R^{1.1C}, —OR^{1.1A}, —NR^{1.1B}SO₂R^{1.1A}, —NR^{1.1B}C(O)R^{1.1D}, —NR^{1.1B}C(O)OR^{1.1D}, —NR^{1.1B}OR^{1.1D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R^{1.2} is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO_{n1.2}H, —SO_{v1.2}NH₂, —NHNH₂, —ONH₂, —NHC(O)NHNH₂, —NHC(O)NH₂, —N(O)_{m1.2}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1.3} is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO_{n1.3}H, —SO_{v1.3}NH₂, —NHNH₂, —ONH₂, —NHC(O)NHNH₂, —NHC(O)NH₂, —N(O)_{m1.3}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1.4} is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO_{n1.4}H, —SO_{v1.4}NH₂, —NHNH₂, —ONH₂, —NHC(O)NHNH₂, —NHC(O)NH₂, —N(O)_{m1.4}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹² is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SOₙ₁₁₂H, —SOᵥ₁₂NH₂, —NHNH₂, —ONH₂, —NHC(O)NHNH₂, —NHC(O)NH₂, —N(O)ₘₗ₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R^{1.1A}, R^{1.1B}, R^{1.1C} and R^{1.1D} are independently hydrogen, halogen, —CF₃, —CCl₃, —CBr₃, —Cl₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1.1B} and R^{1.1C} substituents bonded to the same nitrogen atom may optionally be joined to form a substituted or unsubstituted heterocycloalkyl or substituted or unsubstituted heteroaryl; and

wherein R^{1.1} and R^{1.2} or R^{1.3} and R^{1.4} may optionally be joined to form a substituted or unsubstituted cycloalkyl, substituted or unsubstituted heterocycloalkyl, substituted or unsubstituted aryl or substituted or unsubstituted heteroaryl, and further wherein at least one of R^{1.1}, R^{1.2}, R^{1.3} and R^{1.4} is not hydrogen.

### Embodiment 57

The compound of embodiment 1, wherein the compound is of structural Formula (IV):

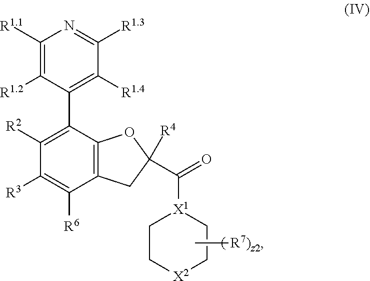

or a pharmaceutically acceptable salt thereof,

wherein:

X¹ is C(R⁸) or N;

X² is C(R⁹)(R¹⁰) or N(R⁹);

n1.1, n1.2, n1.3, n1.4, n7, n8, n9 and n10 are independently and integer from 0 to 4;

m1, m1.2, m1.3, m1.4, m7, m8, m9, m10, v1.1, v1.2, v1.3, v1.4, v7, v8, v9 and v10 are independently 1 or 2;

z2 is an integer from 0 to 8;

R^{1.1} is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO_{n1.1}R^{1.1A}, —SO_{v1.1}NR^{1.1B}R^{1.1C}, —NHNR^{1.1B}R^{1.1C}, —ONR^{1.1B}R^{1.1C}, —NHC(O)NHNR^{1.1B}R^{1.1C}, —NHC(O)NR^{1.1B}R^{1.1C}, —N(O)_{m1.1}, —NR^{1.1B}R^{1.1C}, —C(O)R^{1.1D}, —C(O)OR^{1.1D}, —C(O)NR^{1.1B}R^{1.1C}, —OR^{1.1A}, —NR^{1.1B}SO₂R^{1.1A}, —NR^{1.1B}C(O)R^{1.1D}, —NR^{1.1B}C(O)OR^{1.1D}, —NR^{1.1B}OR^{1.1D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R^{1.2} is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO_{n1.2}H, —SO_{v1.2}NH₂, —NHNH₂, —ONH₂, —NHC(O)NHNH₂, —NHC(O)NH₂, —N(O)_{m1.2}, —NH₂, —C(O)H, —C(O)OH, —C(O)NH₂, —OH, —NHSO₂H, —NHC(O)H, —NHC(O)OH, —NHOH—OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1.3} is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO_{n1.3}H, —SO_{v1.3}NH₂, —NHNH₂, —ONH₂, —NHC(O)NHNH₂, —NHC(O)NH₂, —N(O)_{m1.3}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1.4} is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO_{n1.4}H, —SO_{v1.4}NH₂, —NHNH₂, —ONH₂, —NHC(O)NHNH₂, —NHC(O)NH₂, —N(O)_{m1.4}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R⁷ is independently halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SOₙ₇H, —SOᵥ₇NH₂, —NHNH₂, —ONH₂, —NHC(O)NHNH₂, —NHC(O)NH₂, —N(O)ₘ₇, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R⁸ is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SOₙ₅H, —SOᵥ₅NH₂, —NHNH₂, —ONH₂, —NHC(O)NHNH₂, —NHC(O)NH₂, —N(O)ₘ₅, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R⁹ is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SOₙ₉R^{9A}, —SOᵥ₉NR^{9B}R^{9C}, —NHNR^{9B}R^{9C}, —ONR^{9B}R^{9C}, —NHC(O)NHNR^{9B}R^{9C}, —NHC(O)NR^{9B}R^{9C}, —N(O)ₘ₉, —NR^{9B}R^{9C}, —C(O)R^{9D}, —C(O)OR^{9D}, —C(O)NR^{9B}R^{9C}, —OR^{9A}, —NR^{9B}SO₂R^{9A}, —NR^{9B}C(O)R^{9D}, —NR^{9B}C(O)OR^{9D}, —NR^{9B}OR^{9D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;

R¹⁰ is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SOₙ₁₀R^{10A}, —SOᵥ₁₀NR^{10B}R^{10C}, —NHNR^{10B}R^{10C}, —ONR^{10B}R^{10C}, —NHC(O)NHNR^{10B}R^{10C}, —NHC(O)NR^{10B}R^{10C}, —N(O)^{m10}, —NR^{10B}R^{10C}, —C(O)R^{10D}, —C(O)OR^{10D}, —C(O)NR^{1B}R^{1C}, —OR^{10A}, —NR^{10B}SO₂R^{10A}, —NR^{10B}C(O)R^{10D}, —NR^{10B}C(O)OR^{10D}, —NR^{10B}OR^{10D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and

R^{1.1A}, R^{1.1B}, R^{1.1C}, R^{1.1D}, R^{9A}, R^{9B}, R^{9C}, R^{9A}, R^{10A}, R^{10B}, R^{10C} and R^{10D} are independently hydrogen, halogen, —CF₃, —CCl₃, —CBr₃, —Cl₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1.1B} and R^{1.1C}, R^{9B} and R^{9C} or R^{10B} and R^{10C} substituents bonded to the same nitrogen atom may optionally be joined to form a substituted or unsubstituted heterocycloalkyl or substituted or unsubstituted heteroaryl; and wherein R^{1.1} and R^{1.2} or R^{1.3} and R^{1.4} may optionally be joined to form a substituted or unsubstituted cycloalkyl, substituted or unsubstituted heterocycloalkyl, substituted or unsubstituted aryl or substituted or unsubstituted heteroaryl, and further wherein at least one of R^{1.1}, R^{1.2}, R^{1.3} and R^{1.4} is not hydrogen.

### Embodiment 58

The compound of embodiment 1, wherein the compound is of structural Formula (V):

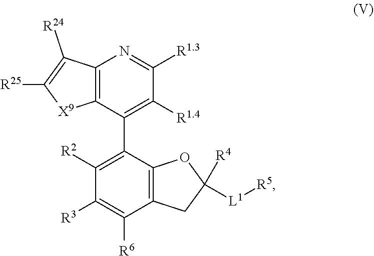

or a pharmaceutically acceptable salt thereof, wherein:

n1.3, n1.4, n24 and n25 are independently and integer from 0 to 4;

m1.3, m1.4, m24, m25, v1.3, v1.4, v24 and v25 are independently 1 or 2;

X⁹ is —O— or —S—;

R^{1.3} is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO_{n1.3}H, —SO_{v1.3}NH₂, —NHNH₂, —ONH₂, —NHC(O)NHNH₂, —NHC(O)NH₂, —N(O)_{m1.3}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1.4} is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO_{n1.4}H, —SO_{1.4}NH₂, —NHNH₂, —ONH₂, —NHC(O)NHNH₂, —NHC(O)NH₂, —N(O)_{m1.4}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁴ is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SOₙ₂₄H, —SOᵥ₂₄NH₂, —NHNH₂, —ONH₂, —NHC(O)NHNH₂, —NHC(O)NH₂, —N(O)ₘ₂₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R²⁵ is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SOₙ₂₅R^{25A}, —SOᵥ₂₅NR^{25B}R^{25C}, —NHNR^{25B}R^{25C}, —ONR^{25B}R^{25C}, —NHC(O)NHNR^{25B}R^{25C}, —NHC(O)NR^{25B}R^{25C}, —N(O)ₘ₂₅, —NR^{25B}R^{25C}, —C(O)R^{25D}, —C(O)OR^{25D}, —C(O)NR^{25B}R^{25C}, —OR^{25A}, —NR^{25B}SO₂R^{25A}, —NR^{25B}C(O)R^{25D}, —NR^{25B}C(O)OR^{25D}, —NR^{25B}OR^{25D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and

R^{25A}, R^{25B}, R^{25C} and R^{25D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{25B} and R^{25C} substituents bonded to the same nitrogen atom may optionally be joined to form a substituted or unsubstituted heterocycloalkyl or substituted or unsubstituted heteroaryl.

### Embodiment 59

The compound of embodiment 1, wherein the compound is of structural Formula (VI):

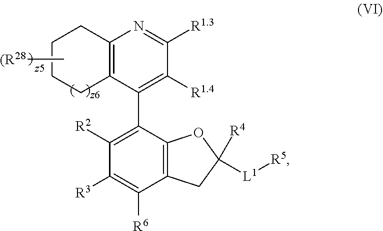

or a pharmaceutically acceptable salt thereof, wherein:

z5 is an integer from 0 to 8;

z6 is 0 or 1;

n1.3, n1.4 and n28 are independently and integer from 0 to 4;

m1.3, m1.4, m28, v1.3, v1.4 and v28 are independently 1 or 2;

R^{1.3} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.3}H, —SO_{v1.3}NH₂, —NHNH₂, —ONH₂, —NHC(O)NHNH₂, —NHC(O)NH₂, —N(O)_{m1.3}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R¹⁴ is hydrogen, halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO_{n1.4}H, —SO_{v1.4}NH₂, —NHNH₂, —ONH₂, —NHC(O)NHNH₂, —NHC(O)NH₂, —N(O)_{m1.4}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

R²⁸ is independently halogen, oxo, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —SO₂₈H, —SOᵥ₂₈NH₂, —NHNH₂, —ONH₂, —NHC(O)NHNH₂, —NHC(O)NH₂, —N(O)ₘ₂₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 60

The compound of any one of embodiments 1-59, wherein the compound is:

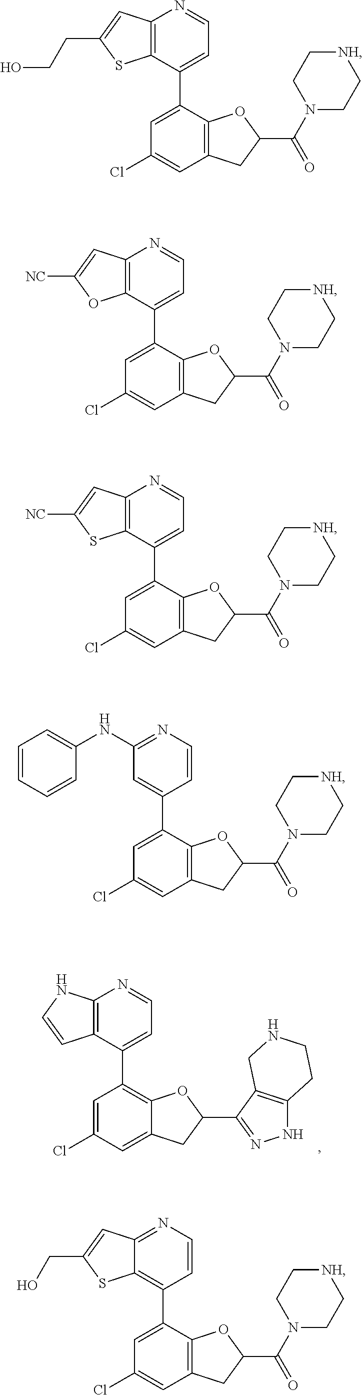 [SMILES: O=C(C1Cc2cc(Cl)cc(-c3ccnc4cc(CCO)sc34)c2O1)N1CCNCC1 ; N#Cc1cc2nccc(-c3cc(Cl)cc4c3OC(C(=O)N3CCNCC3)C4)c2o1 ; N#Cc1cc2nccc(-c3cc(Cl)cc4c3OC(C(=O)N3CCNCC3)C4)c2s1 ; O=C(C1Cc2cc(Cl)cc(-c3ccnc(Nc4ccccc4)c3)c2O1)N1CCNCC1 ; Clc1cc2c(c(-c3ccnc4[nH]ccc34)c1)OC(c1n[nH]c3c1CNCC3)C2 ; O=C(C1Cc2cc(Cl)cc(-c3ccnc4cc(CO)sc34)c2O1)N1CCNCC1]

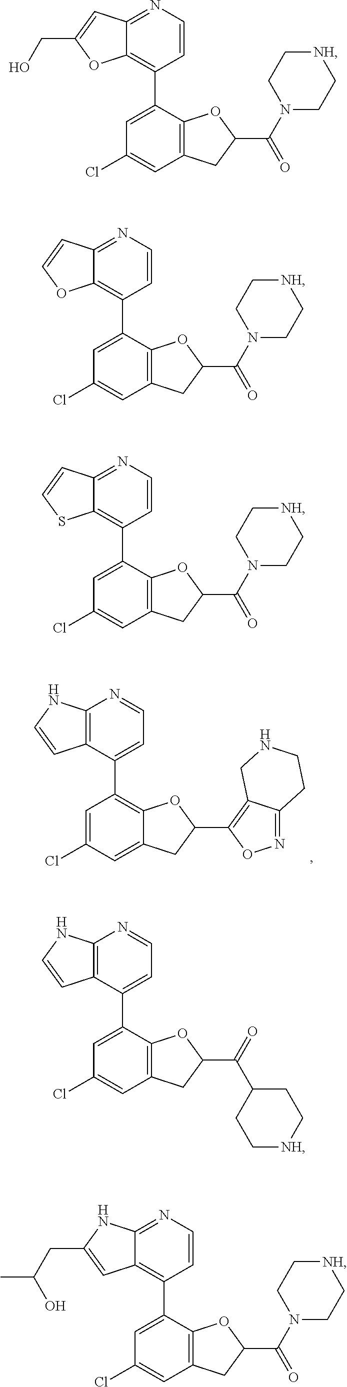 [SMILES: O=C(C1Cc2cc(Cl)cc(-c3ccnc4cc(CO)oc34)c2O1)N1CCNCC1 ; O=C(C1Cc2cc(Cl)cc(-c3ccnc4ccoc34)c2O1)N1CCNCC1 ; O=C(C1Cc2cc(Cl)cc(-c3ccnc4ccsc34)c2O1)N1CCNCC1 ; Clc1cc2c(c(-c3ccnc4[nH]ccc34)c1)OC(c1onc3c1CNCC3)C2 ; O=C(C1CCNCC1)C1Cc2cc(Cl)cc(-c3ccnc4[nH]ccc34)c2O1 ; CC(O)Cc1cc2c(-c3cc(Cl)cc4c3OC(C(=O)N3CCNCC3)C4)ccnc2[nH]1]

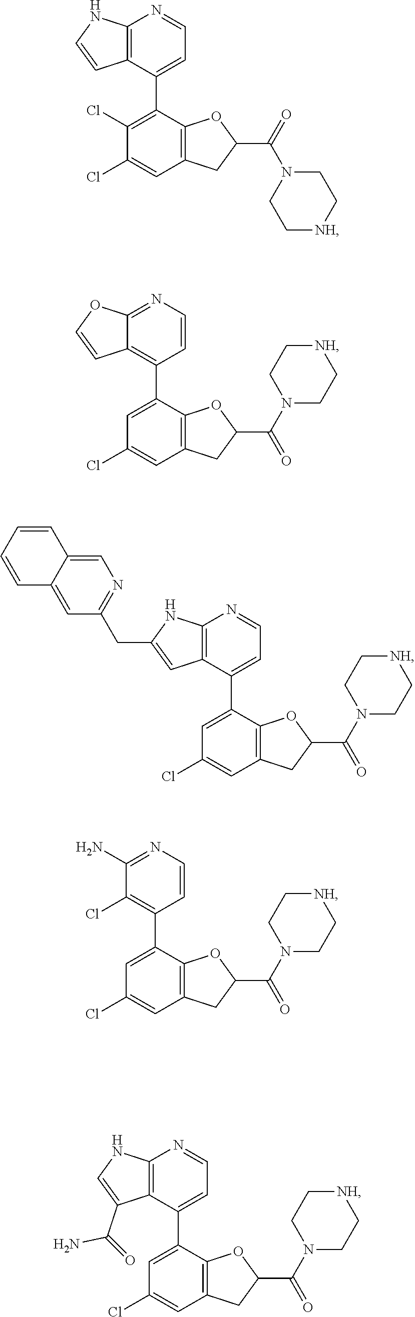 [SMILES: O=C(C1Cc2cc(Cl)c(Cl)c(-c3ccnc4[nH]ccc34)c2O1)N1CCNCC1 ; O=C(C1Cc2cc(Cl)cc(-c3ccnc4occc34)c2O1)N1CCNCC1 ; O=C(C1Cc2cc(Cl)cc(-c3ccnc4[nH]c(Cc5cc6ccccc6cn5)cc34)c2O1)N1CCNCC1 ; Nc1nccc(-c2cc(Cl)cc3c2OC(C(=O)N2CCNCC2)C3)c1Cl ; NC(=O)c1c[nH]c2nccc(-c3cc(Cl)cc4c3OC(C(=O)N3CCNCC3)C4)c12]

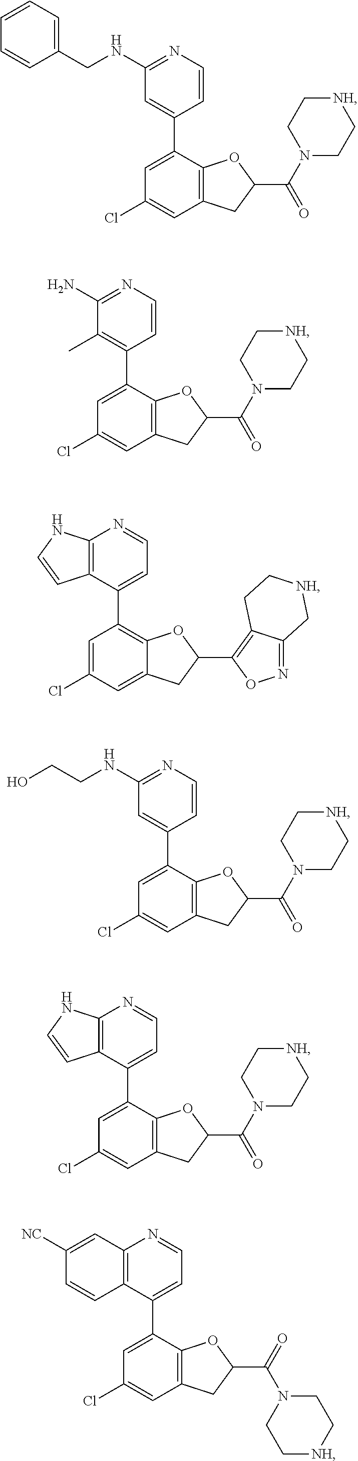 [SMILES: O=C(C1Cc2cc(Cl)cc(-c3ccnc(NCc4ccccc4)c3)c2O1)N1CCNCC1 ; Cc1c(-c2cc(Cl)cc3c2OC(C(=O)N2CCNCC2)C3)ccnc1N ; Clc1cc2c(c(-c3ccnc4[nH]ccc34)c1)OC(c1onc3c1CCNC3)C2 ; O=C(C1Cc2cc(Cl)cc(-c3ccnc(NCCO)c3)c2O1)N1CCNCC1 ; O=C(C1Cc2cc(Cl)cc(-c3ccnc4[nH]ccc34)c2O1)N1CCNCC1 ; N#Cc1ccc2c(-c3cc(Cl)cc4c3OC(C(=O)N3CCNCC3)C4)ccnc2c1]

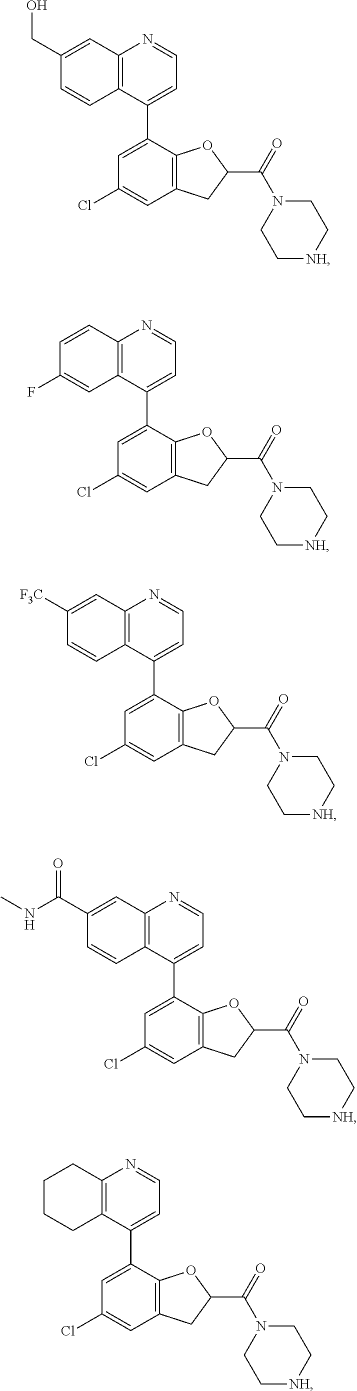 [SMILES: O=C(C1Cc2cc(Cl)cc(-c3ccnc4cc(CO)ccc34)c2O1)N1CCNCC1 ; O=C(C1Cc2cc(Cl)cc(-c3ccnc4ccc(F)cc34)c2O1)N1CCNCC1 ; O=C(C1Cc2cc(Cl)cc(-c3ccnc4cc(C(F)(F)F)ccc34)c2O1)N1CCNCC1 ; CNC(=O)c1ccc2c(-c3cc(Cl)cc4c3OC(C(=O)N3CCNCC3)C4)ccnc2c1 ; O=C(C1Cc2cc(Cl)cc(-c3ccnc4c3CCCC4)c2O1)N1CCNCC1]

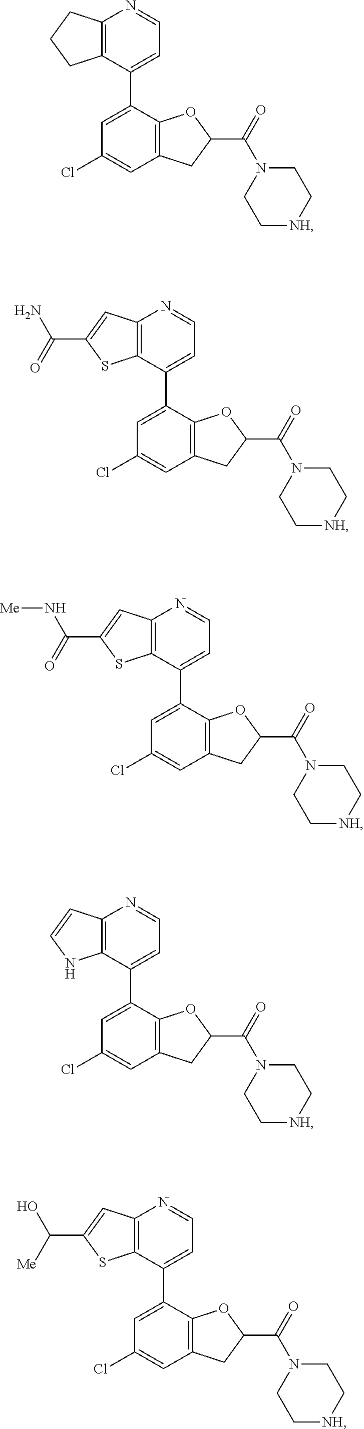 [SMILES: O=C(C1Cc2cc(Cl)cc(-c3ccnc4c3CCC4)c2O1)N1CCNCC1 ; NC(=O)c1cc2nccc(-c3cc(Cl)cc4c3OC(C(=O)N3CCNCC3)C4)c2s1 ; CNC(=O)c1cc2nccc(-c3cc(Cl)cc4c3OC(C(=O)N3CCNCC3)C4)c2s1 ; O=C(C1Cc2cc(Cl)cc(-c3ccnc4cc[nH]c34)c2O1)N1CCNCC1 ; CC(O)c1cc2nccc(-c3cc(Cl)cc4c3OC(C(=O)N3CCNCC3)C4)c2s1]

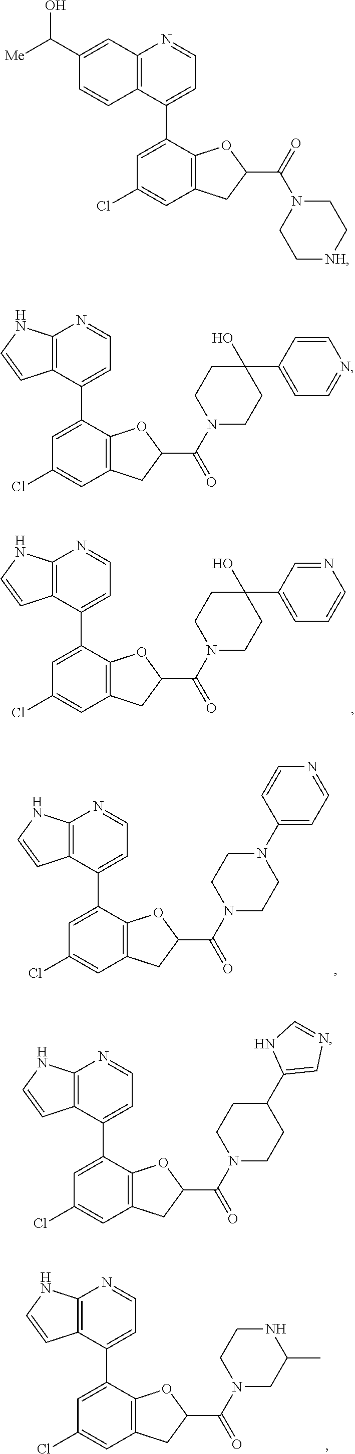 [SMILES: CC(O)c1ccc2c(-c3cc(Cl)cc4c3OC(C(=O)N3CCNCC3)C4)ccnc2c1 ; O=C(C1Cc2cc(Cl)cc(-c3ccnc4[nH]ccc34)c2O1)N1CCC(O)(c2ccncc2)CC1 ; O=C(C1Cc2cc(Cl)cc(-c3ccnc4[nH]ccc34)c2O1)N1CCC(O)(c2cccnc2)CC1 ; O=C(C1Cc2cc(Cl)cc(-c3ccnc4[nH]ccc34)c2O1)N1CCN(c2ccncc2)CC1 ; O=C(C1Cc2cc(Cl)cc(-c3ccnc4[nH]ccc34)c2O1)N1CCC(c2cnc[nH]2)CC1 ; CC1CN(C(=O)C2Cc3cc(Cl)cc(-c4ccnc5[nH]ccc45)c3O2)CCN1]

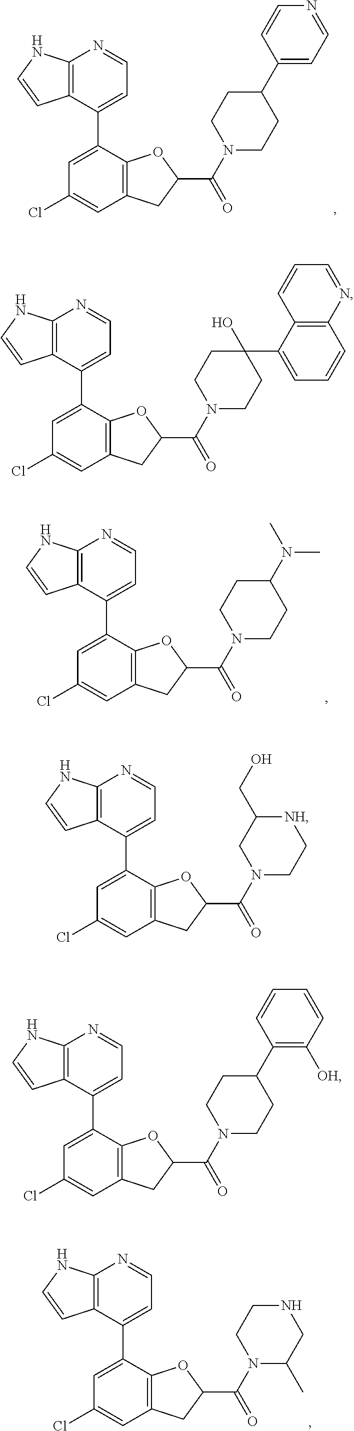 [SMILES: O=C(C1Cc2cc(Cl)cc(-c3ccnc4[nH]ccc34)c2O1)N1CCC(c2ccncc2)CC1 ; O=C(C1Cc2cc(Cl)cc(-c3ccnc4[nH]ccc34)c2O1)N1CCC(O)(c2cccc3ncccc23)CC1 ; CN(C)C1CCN(C(=O)C2Cc3cc(Cl)cc(-c4ccnc5[nH]ccc45)c3O2)CC1 ; O=C(C1Cc2cc(Cl)cc(-c3ccnc4[nH]ccc34)c2O1)N1CCNC(CO)C1 ; O=C(C1Cc2cc(Cl)cc(-c3ccnc4[nH]ccc34)c2O1)N1CCC(c2ccccc2O)CC1 ; CC1CNCCN1C(=O)C1Cc2cc(Cl)cc(-c3ccnc4[nH]ccc34)c2O1]

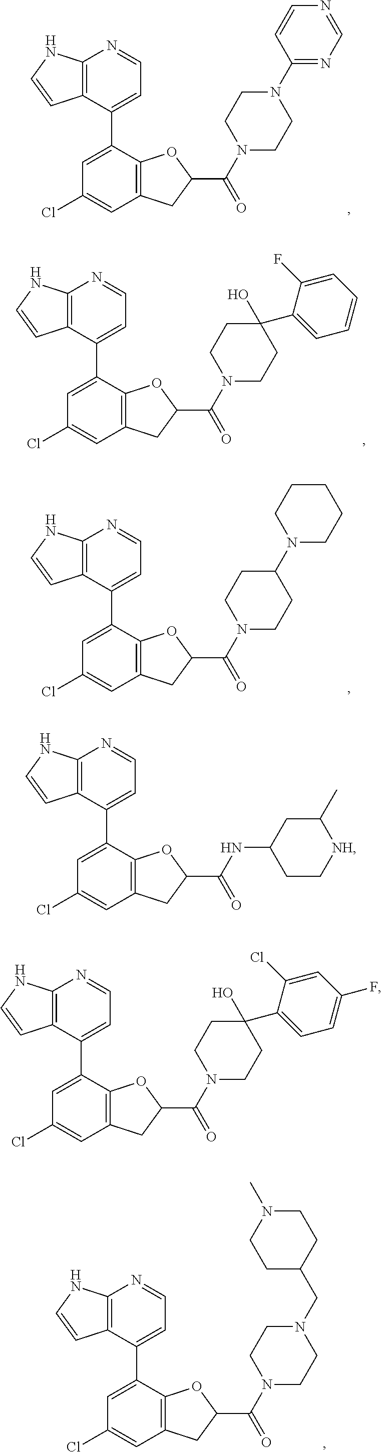 [SMILES: O=C(C1Cc2cc(Cl)cc(-c3ccnc4[nH]ccc34)c2O1)N1CCN(c2ccncn2)CC1 ; O=C(C1Cc2cc(Cl)cc(-c3ccnc4[nH]ccc34)c2O1)N1CCC(O)(c2ccccc2F)CC1 ; O=C(C1Cc2cc(Cl)cc(-c3ccnc4[nH]ccc34)c2O1)N1CCC(N2CCCCC2)CC1 ; CC1CC(NC(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(O)(c2ccc(F)cc2Cl)CC1 ; CN1CCC(CN2CCN(C(=O)C3Cc4cc(Cl)cc(-c5ccnc6[nH]ccc56)c4O3)CC2)CC1]

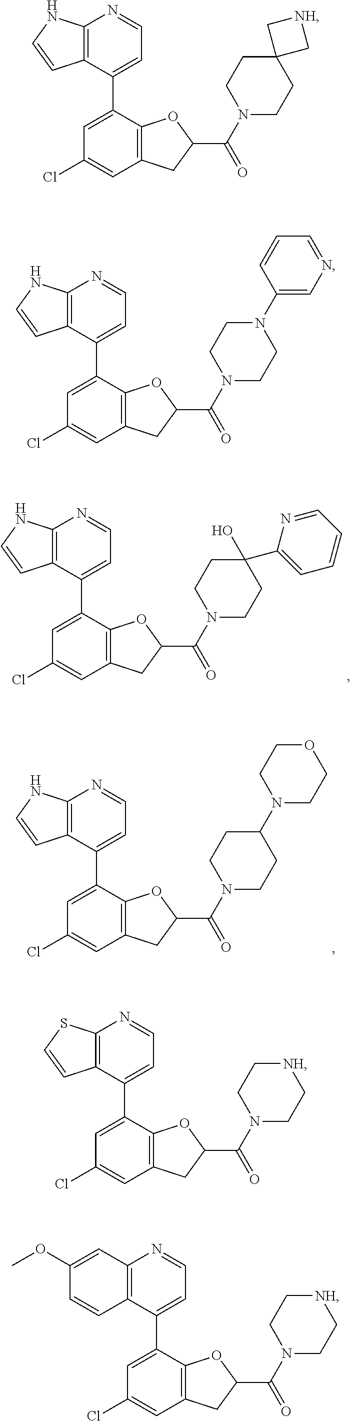 [SMILES: O=C(C1Cc2cc(Cl)cc(-c3ccnc4[nH]ccc34)c2O1)N1CCC2(CC1)CNC2 ; O=C(C1Cc2cc(Cl)cc(-c3ccnc4[nH]ccc34)c2O1)N1CCN(c2cccnc2)CC1 ; O=C(C1Cc2cc(Cl)cc(-c3ccnc4[nH]ccc34)c2O1)N1CCC(O)(c2ccccn2)CC1 ; O=C(C1Cc2cc(Cl)cc(-c3ccnc4[nH]ccc34)c2O1)N1CCC(N2CCOCC2)CC1 ; O=C(C1Cc2cc(Cl)cc(-c3ccnc4sccc34)c2O1)N1CCNCC1 ; COc1ccc2c(-c3cc(Cl)cc4c3OC(C(=O)N3CCNCC3)C4)ccnc2c1]

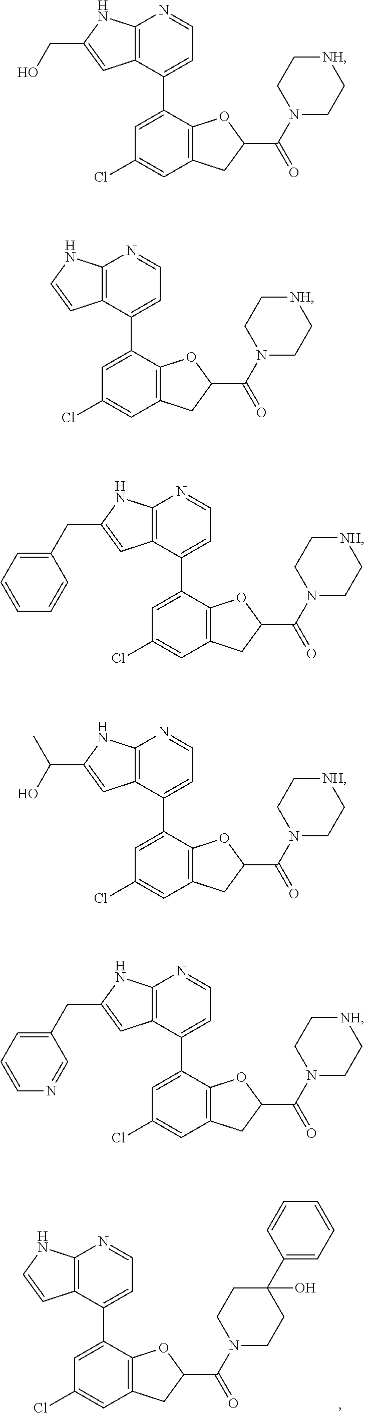 [SMILES: O=C(C1Cc2cc(Cl)cc(-c3ccnc4[nH]c(CO)cc34)c2O1)N1CCNCC1 ; O=C(C1Cc2cc(Cl)cc(-c3ccnc4[nH]ccc34)c2O1)N1CCNCC1 ; O=C(C1Cc2cc(Cl)cc(-c3ccnc4[nH]c(Cc5ccccc5)cc34)c2O1)N1CCNCC1 ; CC(O)c1cc2c(-c3cc(Cl)cc4c3OC(C(=O)N3CCNCC3)C4)ccnc2[nH]1 ; O=C(C1Cc2cc(Cl)cc(-c3ccnc4[nH]c(Cc5cccnc5)cc34)c2O1)N1CCNCC1 ; O=C(C1Cc2cc(Cl)cc(-c3ccnc4[nH]ccc34)c2O1)N1CCC(O)(c2ccccc2)CC1]

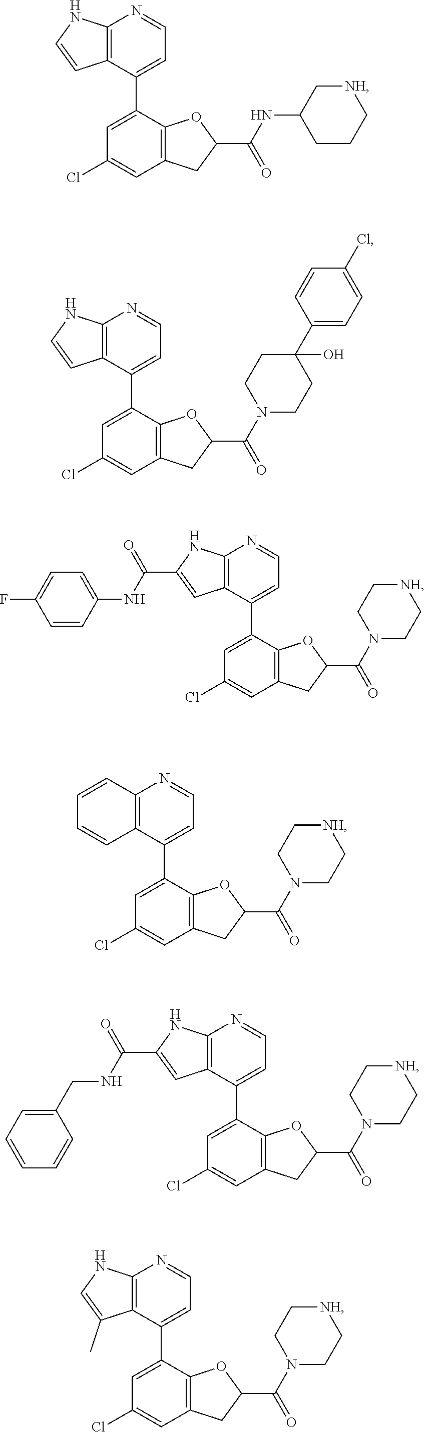 [SMILES: O=C(NC1CCCNC1)C1Cc2cc(Cl)cc(-c3ccnc4[nH]ccc34)c2O1 ; O=C(C1Cc2cc(Cl)cc(-c3ccnc4[nH]ccc34)c2O1)N1CCC(O)(c2ccc(Cl)cc2)CC1 ; O=C(Nc1ccc(F)cc1)c1cc2c(-c3cc(Cl)cc4c3OC(C(=O)N3CCNCC3)C4)ccnc2[nH]1 ; O=C(C1Cc2cc(Cl)cc(-c3ccnc4ccccc34)c2O1)N1CCNCC1 ; O=C(NCc1ccccc1)c1cc2c(-c3cc(Cl)cc4c3OC(C(=O)N3CCNCC3)C4)ccnc2[nH]1 ; Cc1c[nH]c2nccc(-c3cc(Cl)cc4c3OC(C(=O)N3CCNCC3)C4)c12]

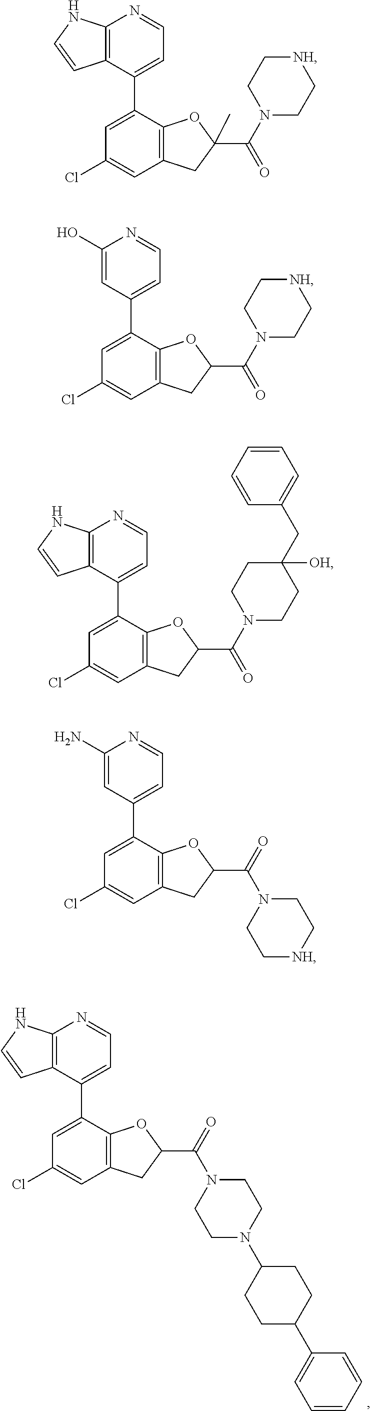 [SMILES: CC1(C(=O)N2CCNCC2)Cc2cc(Cl)cc(-c3ccnc4[nH]ccc34)c2O1 ; O=C(C1Cc2cc(Cl)cc(-c3ccnc(O)c3)c2O1)N1CCNCC1 ; O=C(C1Cc2cc(Cl)cc(-c3ccnc4[nH]ccc34)c2O1)N1CCC(O)(Cc2ccccc2)CC1 ; Nc1cc(-c2cc(Cl)cc3c2OC(C(=O)N2CCNCC2)C3)ccn1 ; O=C(C1Cc2cc(Cl)cc(-c3ccnc4[nH]ccc34)c2O1)N1CCN(C2CCC(c3ccccc3)CC2)CC1]

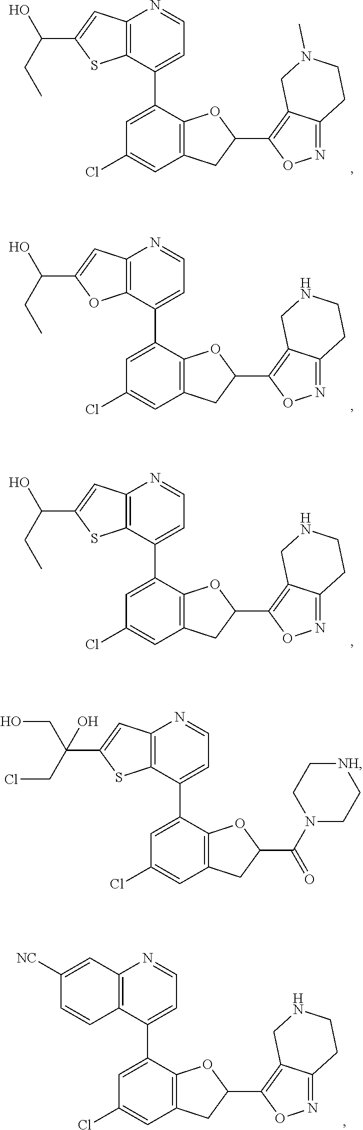 [SMILES: CCC(O)c1cc2nccc(-c3cc(Cl)cc4c3OC(c3onc5c3CN(C)CC5)C4)c2s1 ; CCC(O)c1cc2nccc(-c3cc(Cl)cc4c3OC(c3onc5c3CNCC5)C4)c2o1 ; CCC(O)c1cc2nccc(-c3cc(Cl)cc4c3OC(c3onc5c3CNCC5)C4)c2s1 ; O=C(C1Cc2cc(Cl)cc(-c3ccnc4cc(C(O)(CO)CCl)sc34)c2O1)N1CCNCC1 ; N#Cc1ccc2c(-c3cc(Cl)cc4c3OC(c3onc5c3CNCC5)C4)ccnc2c1]

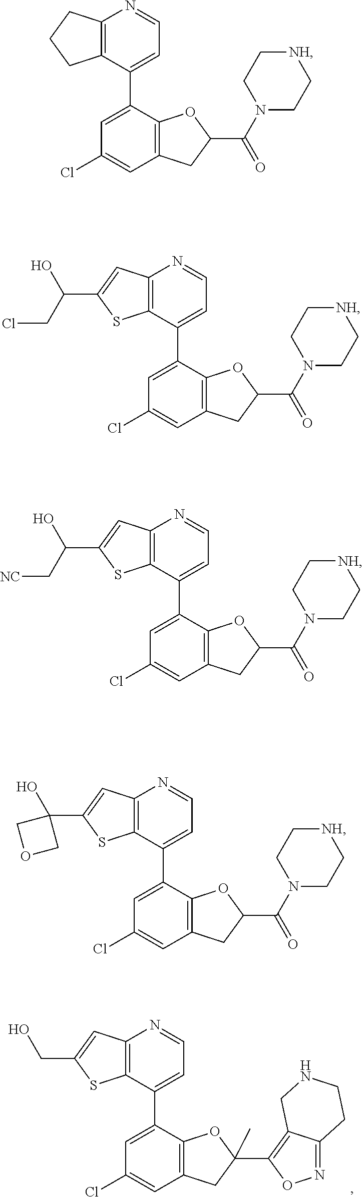 [SMILES: O=C(C1Cc2cc(Cl)cc(-c3ccnc4c3CCC4)c2O1)N1CCNCC1 ; O=C(C1Cc2cc(Cl)cc(-c3ccnc4cc(C(O)CCl)sc34)c2O1)N1CCNCC1 ; N#CCC(O)c1cc2nccc(-c3cc(Cl)cc4c3OC(C(=O)N3CCNCC3)C4)c2s1 ; O=C(C1Cc2cc(Cl)cc(-c3ccnc4cc(C5(O)COC5)sc34)c2O1)N1CCNCC1 ; CC1(c2onc3c2CNCC3)Cc2cc(Cl)cc(-c3ccnc4cc(CO)sc34)c2O1]

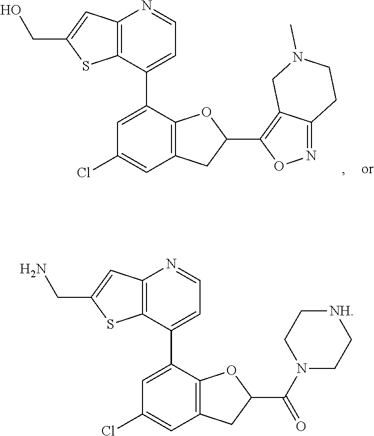 [SMILES: CN1CCc2noc(C3Cc4cc(Cl)cc(-c5ccnc6cc(CO)sc56)c4O3)c2C1 ; NCc1cc2nccc(-c3cc(Cl)cc4c3OC(C(=O)N3CCNCC3)C4)c2s1]

### Embodiment 62

The compound of any one of embodiments 1-59, wherein the compound is:

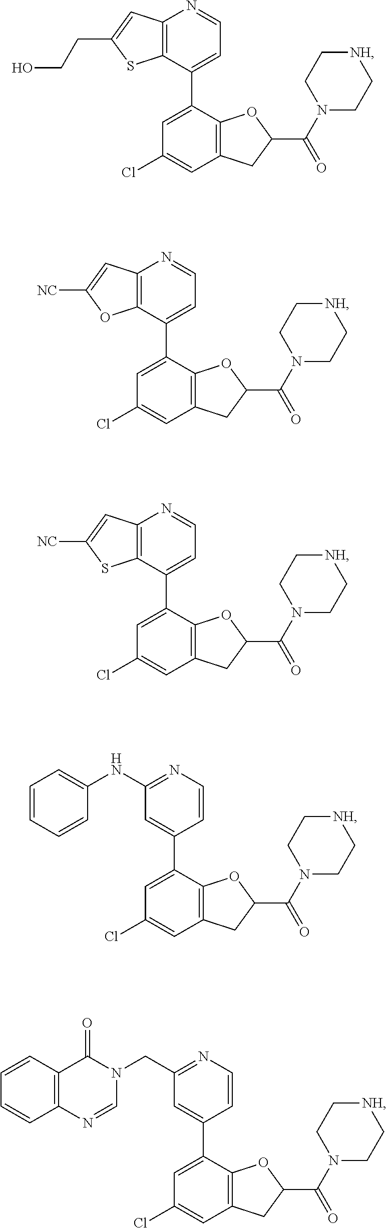 [SMILES: O=C(C1Cc2cc(Cl)cc(-c3ccnc4cc(CCO)sc34)c2O1)N1CCNCC1 ; N#Cc1cc2nccc(-c3cc(Cl)cc4c3OC(C(=O)N3CCNCC3)C4)c2o1 ; N#Cc1cc2nccc(-c3cc(Cl)cc4c3OC(C(=O)N3CCNCC3)C4)c2s1 ; O=C(C1Cc2cc(Cl)cc(-c3ccnc(Nc4ccccc4)c3)c2O1)N1CCNCC1 ; O=C(C1Cc2cc(Cl)cc(-c3ccnc(Cn4cnc5ccccc5c4=O)c3)c2O1)N1CCNCC1]

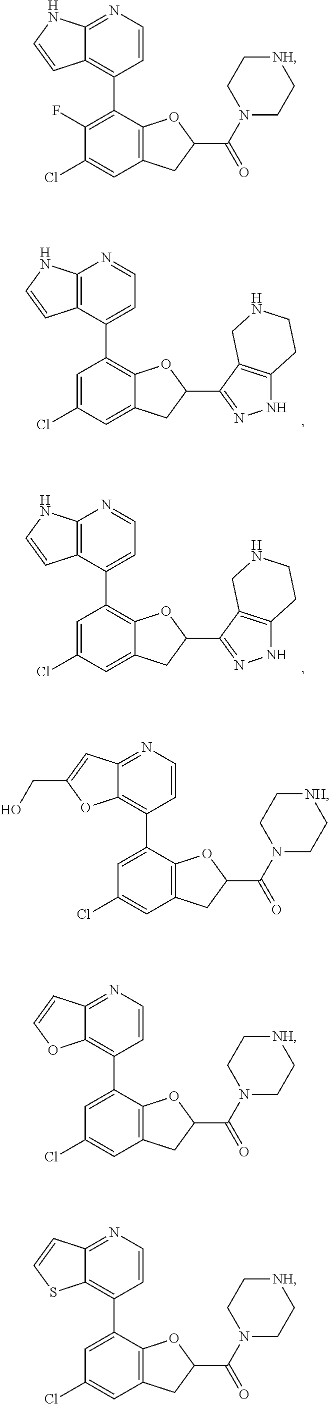 [SMILES: O=C(C1Cc2cc(Cl)c(F)c(-c3ccnc4[nH]ccc34)c2O1)N1CCNCC1 ; Clc1cc2c(c(-c3ccnc4[nH]ccc34)c1)OC(c1n[nH]c3c1CNCC3)C2 ; Clc1cc2c(c(-c3ccnc4[nH]ccc34)c1)OC(c1n[nH]c3c1CNCC3)C2 ; O=C(C1Cc2cc(Cl)cc(-c3ccnc4cc(CO)oc34)c2O1)N1CCNCC1 ; O=C(C1Cc2cc(Cl)cc(-c3ccnc4ccoc34)c2O1)N1CCNCC1 ; O=C(C1Cc2cc(Cl)cc(-c3ccnc4ccsc34)c2O1)N1CCNCC1]

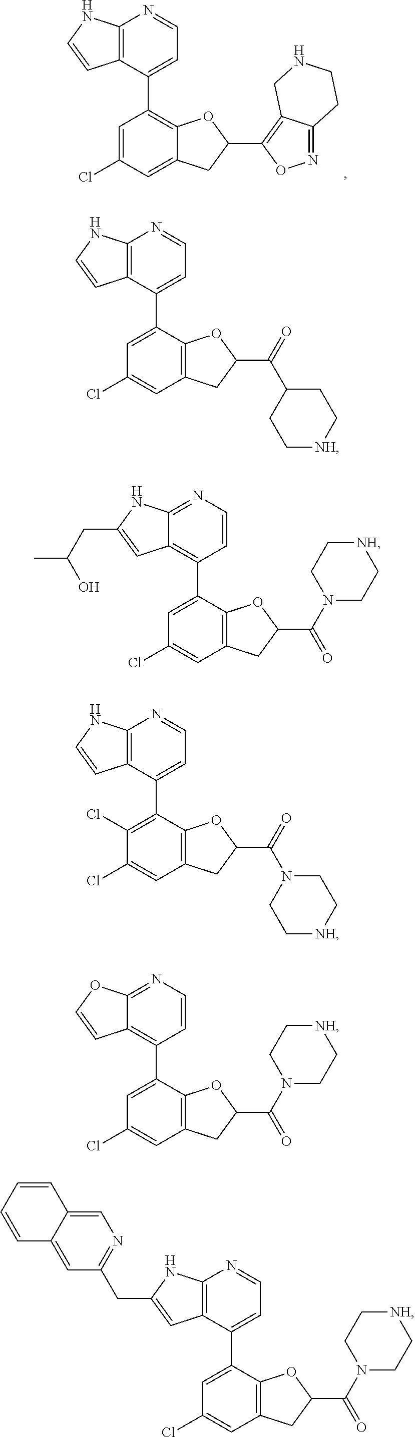 [SMILES: Clc1cc2c(c(-c3ccnc4[nH]ccc34)c1)OC(c1onc3c1CNCC3)C2 ; O=C(C1CCNCC1)C1Cc2cc(Cl)cc(-c3ccnc4[nH]ccc34)c2O1 ; CC(O)Cc1cc2c(-c3cc(Cl)cc4c3OC(C(=O)N3CCNCC3)C4)ccnc2[nH]1 ; O=C(C1Cc2cc(Cl)c(Cl)c(-c3ccnc4[nH]ccc34)c2O1)N1CCNCC1 ; O=C(C1Cc2cc(Cl)cc(-c3ccnc4occc34)c2O1)N1CCNCC1 ; O=C(C1Cc2cc(Cl)cc(-c3ccnc4[nH]c(Cc5cc6ccccc6cn5)cc34)c2O1)N1CCNCC1]

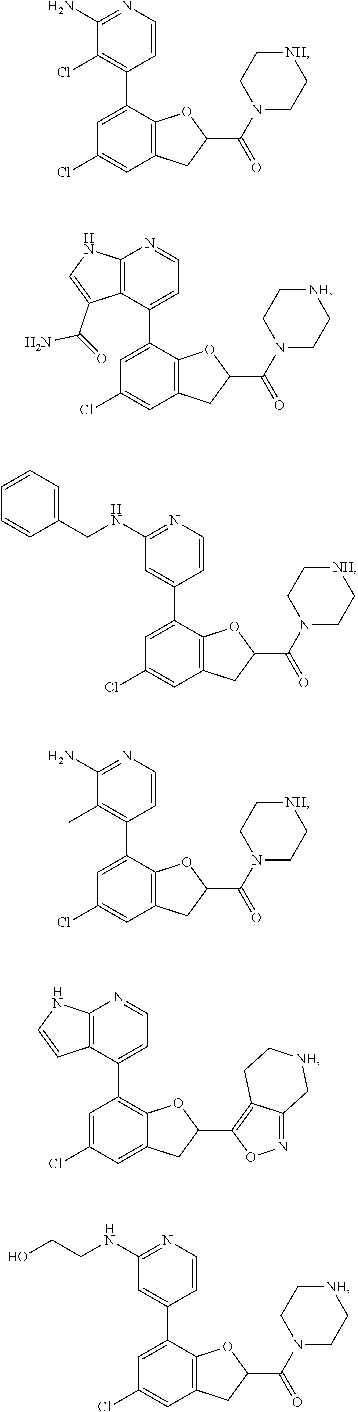 [SMILES: Nc1nccc(-c2cc(Cl)cc3c2OC(C(=O)N2CCNCC2)C3)c1Cl ; NC(=O)c1c[nH]c2nccc(-c3cc(Cl)cc4c3OC(C(=O)N3CCNCC3)C4)c12 ; O=C(C1Cc2cc(Cl)cc(-c3ccnc(NCc4ccccc4)c3)c2O1)N1CCNCC1 ; Cc1c(-c2cc(Cl)cc3c2OC(C(=O)N2CCNCC2)C3)ccnc1N ; Clc1cc2c(c(-c3ccnc4[nH]ccc34)c1)OC(c1onc3c1CCNC3)C2 ; O=C(C1Cc2cc(Cl)cc(-c3ccnc(NCCO)c3)c2O1)N1CCNCC1]

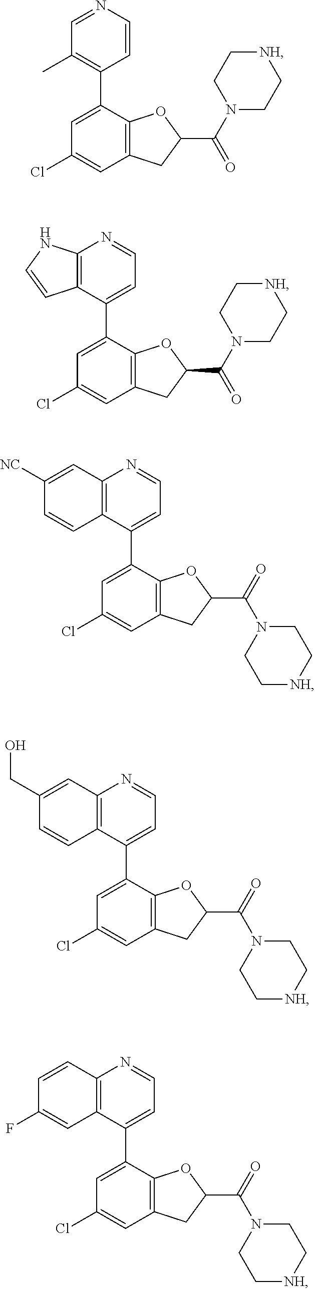 [SMILES: Cc1cnccc1-c1cc(Cl)cc2c1OC(C(=O)N1CCNCC1)C2 ; O=C([C@H]1Cc2cc(Cl)cc(-c3ccnc4[nH]ccc34)c2O1)N1CCNCC1 ; N#Cc1ccc2c(-c3cc(Cl)cc4c3OC(C(=O)N3CCNCC3)C4)ccnc2c1 ; O=C(C1Cc2cc(Cl)cc(-c3ccnc4cc(CO)ccc34)c2O1)N1CCNCC1 ; O=C(C1Cc2cc(Cl)cc(-c3ccnc4ccc(F)cc34)c2O1)N1CCNCC1]

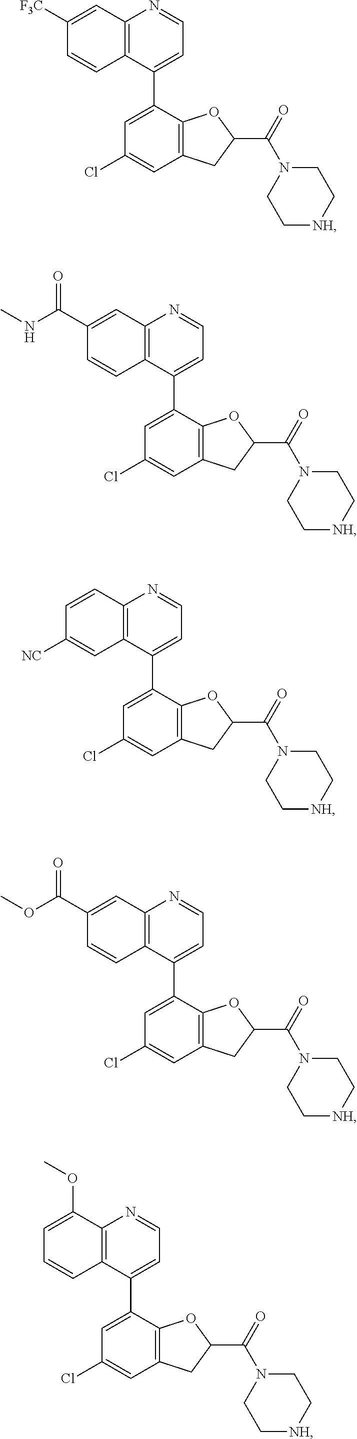 [SMILES: O=C(C1Cc2cc(Cl)cc(-c3ccnc4cc(C(F)(F)F)ccc34)c2O1)N1CCNCC1 ; CNC(=O)c1ccc2c(-c3cc(Cl)cc4c3OC(C(=O)N3CCNCC3)C4)ccnc2c1 ; N#Cc1ccc2nccc(-c3cc(Cl)cc4c3OC(C(=O)N3CCNCC3)C4)c2c1 ; COC(=O)c1ccc2c(-c3cc(Cl)cc4c3OC(C(=O)N3CCNCC3)C4)ccnc2c1 ; COc1cccc2c(-c3cc(Cl)cc4c3OC(C(=O)N3CCNCC3)C4)ccnc12]

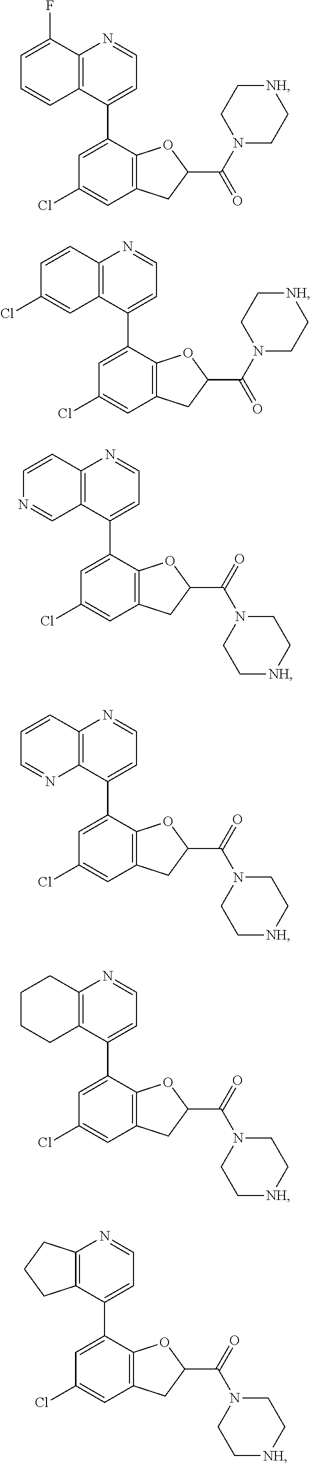

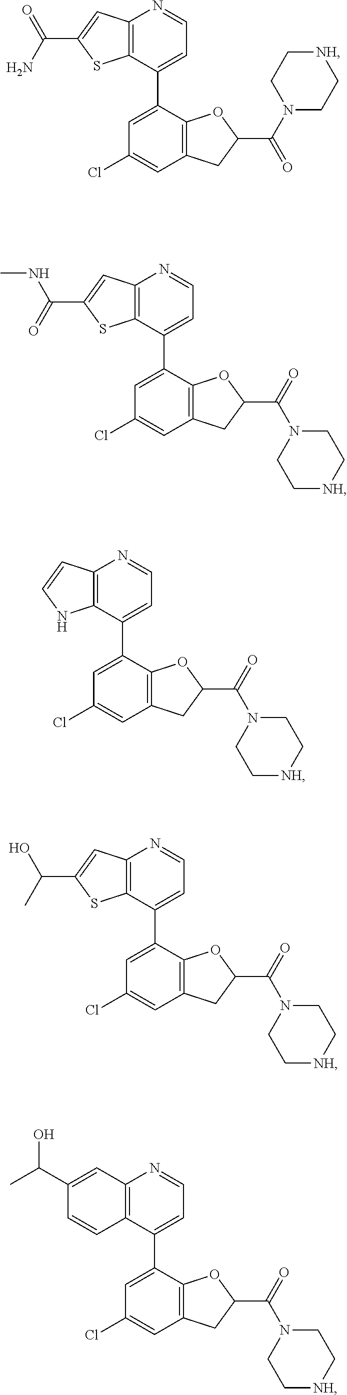 [SMILES: NC(=O)c1cc2nccc(-c3cc(Cl)cc4c3OC(C(=O)N3CCNCC3)C4)c2s1 ; CNC(=O)c1cc2nccc(-c3cc(Cl)cc4c3OC(C(=O)N3CCNCC3)C4)c2s1 ; O=C(C1Cc2cc(Cl)cc(-c3ccnc4cc[nH]c34)c2O1)N1CCNCC1 ; CC(O)c1cc2nccc(-c3cc(Cl)cc4c3OC(C(=O)N3CCNCC3)C4)c2s1 ; CC(O)c1ccc2c(-c3cc(Cl)cc4c3OC(C(=O)N3CCNCC3)C4)ccnc2c1]

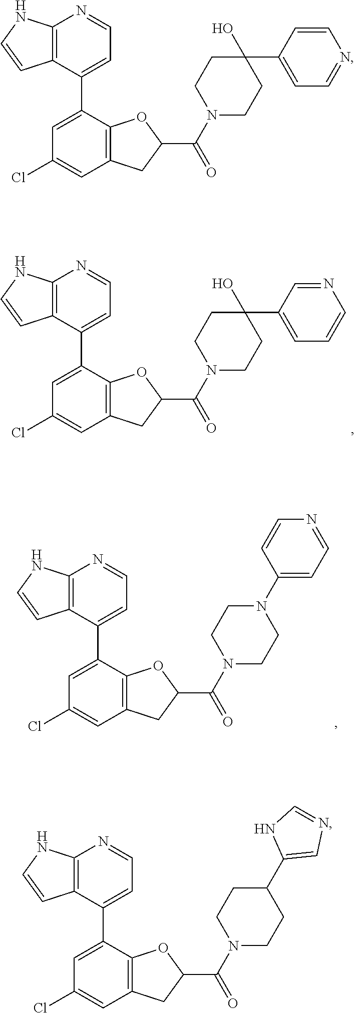 [SMILES: O=C(C1Cc2cc(Cl)cc(-c3ccnc4[nH]ccc34)c2O1)N1CCC(O)(c2ccncc2)CC1 ; O=C(C1Cc2cc(Cl)cc(-c3ccnc4[nH]ccc34)c2O1)N1CCC(O)(c2cccnc2)CC1 ; O=C(C1Cc2cc(Cl)cc(-c3ccnc4[nH]ccc34)c2O1)N1CCN(c2ccncc2)CC1 ; O=C(C1Cc2cc(Cl)cc(-c3ccnc4[nH]ccc34)c2O1)N1CCC(c2cnc[nH]2)CC1]

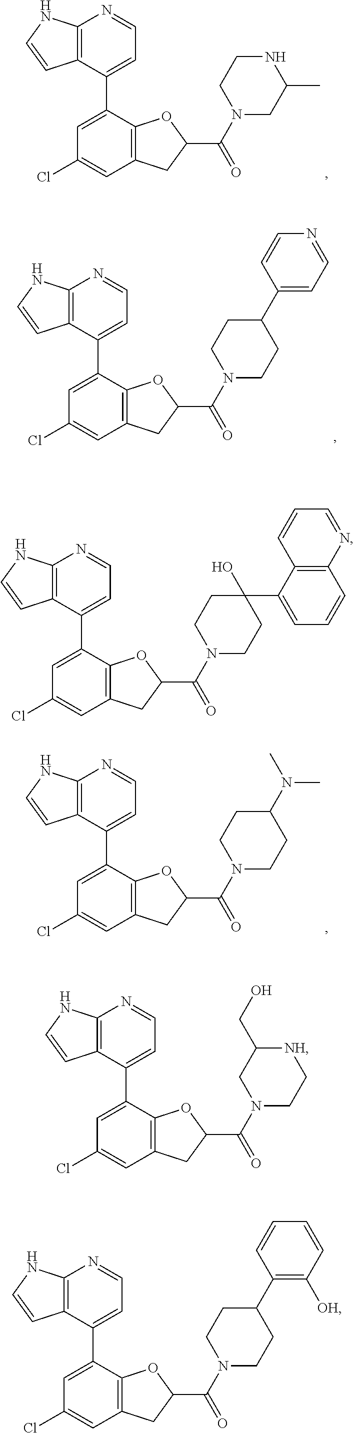 [SMILES: CC1CN(C(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(c2ccncc2)CC1 ; O=C(C1Cc2cc(Cl)cc(-c3ccnc4[nH]ccc34)c2O1)N1CCC(O)(c2cccc3ncccc23)CC1 ; CN(C)C1CCN(C(=O)C2Cc3cc(Cl)cc(-c4ccnc5[nH]ccc45)c3O2)CC1 ; O=C(C1Cc2cc(Cl)cc(-c3ccnc4[nH]ccc34)c2O1)N1CCNC(CO)C1 ; O=C(C1Cc2cc(Cl)cc(-c3ccnc4[nH]ccc34)c2O1)N1CCC(c2ccccc2O)CC1]

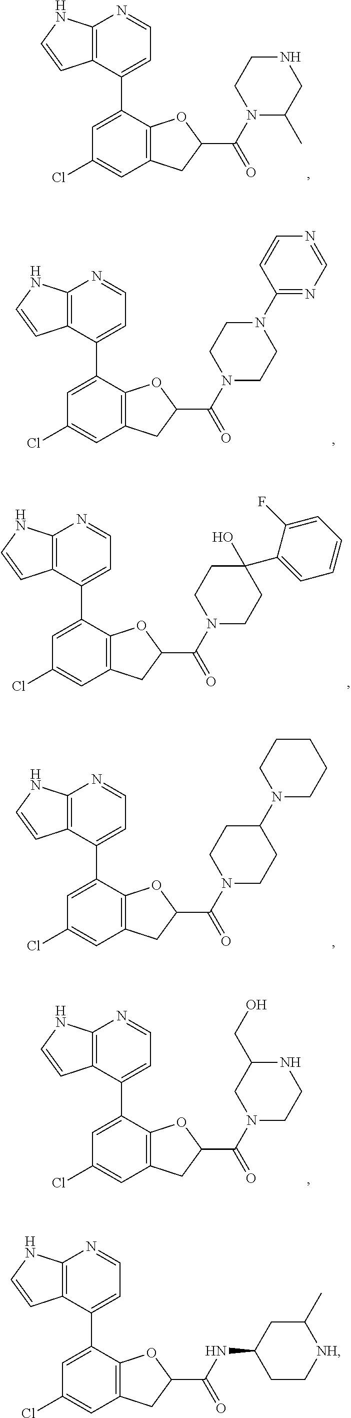 [SMILES: CC1CNCCN1C(=O)C1Cc2cc(Cl)cc(-c3ccnc4[nH]ccc34)c2O1 ; O=C(C1Cc2cc(Cl)cc(-c3ccnc4[nH]ccc34)c2O1)N1CCN(c2ccncn2)CC1 ; O=C(C1Cc2cc(Cl)cc(-c3ccnc4[nH]ccc34)c2O1)N1CCC(O)(c2ccccc2F)CC1 ; O=C(C1Cc2cc(Cl)cc(-c3ccnc4[nH]ccc34)c2O1)N1CCC(N2CCCCC2)CC1 ; O=C(C1Cc2cc(Cl)cc(-c3ccnc4[nH]ccc34)c2O1)N1CCNC(CO)C1 ; CC1C[C@H](NC(=O)C2Cc3cc(Cl)cc(-c4ccnc5[nH]ccc45)c3O2)CCN1]

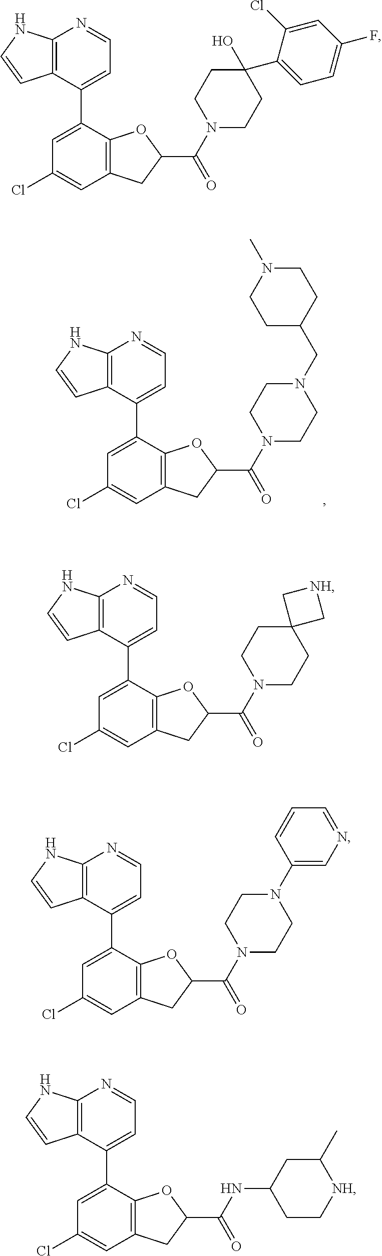 [SMILES: O=C(C1Cc2cc(Cl)cc(-c3ccnc4[nH]ccc34)c2O1)N1CCC(O)(c2ccc(F)cc2Cl)CC1 ; CN1CCC(CN2CCN(C(=O)C3Cc4cc(Cl)cc(-c5ccnc6[nH]ccc56)c4O3)CC2)CC1 ; O=C(C1Cc2cc(Cl)cc(-c3ccnc4[nH]ccc34)c2O1)N1CCC2(CC1)CNC2 ; O=C(C1Cc2cc(Cl)cc(-c3ccnc4[nH]ccc34)c2O1)N1CCN(c2cccnc2)CC1 ; CC1CC(NC(=O)C2Cc3cc(Cl)cc(-c4ccnc5[nH]ccc45)c3O2)CCN1]

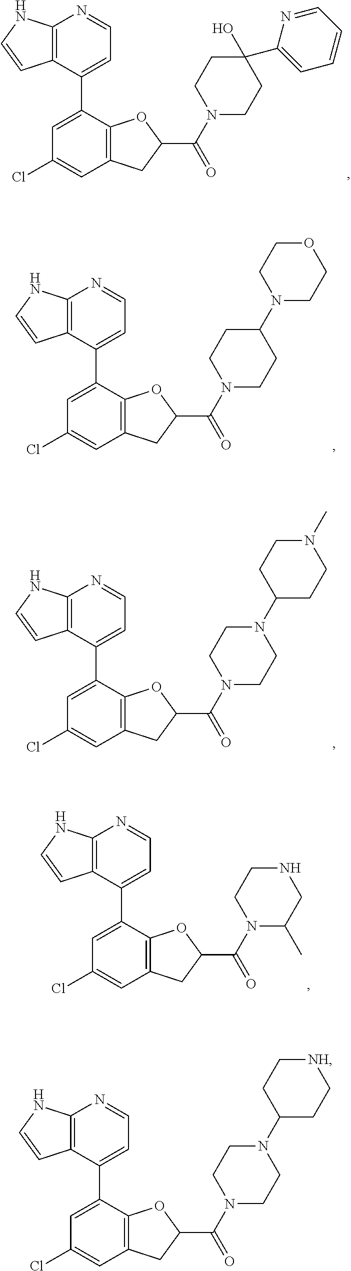 [SMILES: O=C(C1Cc2cc(Cl)cc(-c3ccnc4[nH]ccc34)c2O1)N1CCC(O)(c2ccccn2)CC1 ; O=C(C1Cc2cc(Cl)cc(-c3ccnc4[nH]ccc34)c2O1)N1CCC(N2CCOCC2)CC1 ; CN1CCC(N2CCN(C(=O)C3Cc4cc(Cl)cc(-c5ccnc6[nH]ccc56)c4O3)CC2)CC1 ; CC1CNCCN1C(=O)C1Cc2cc(Cl)cc(-c3ccnc4[nH]ccc34)c2O1 ; O=C(C1Cc2cc(Cl)cc(-c3ccnc4[nH]ccc34)c2O1)N1CCN(C2CCNCC2)CC1]

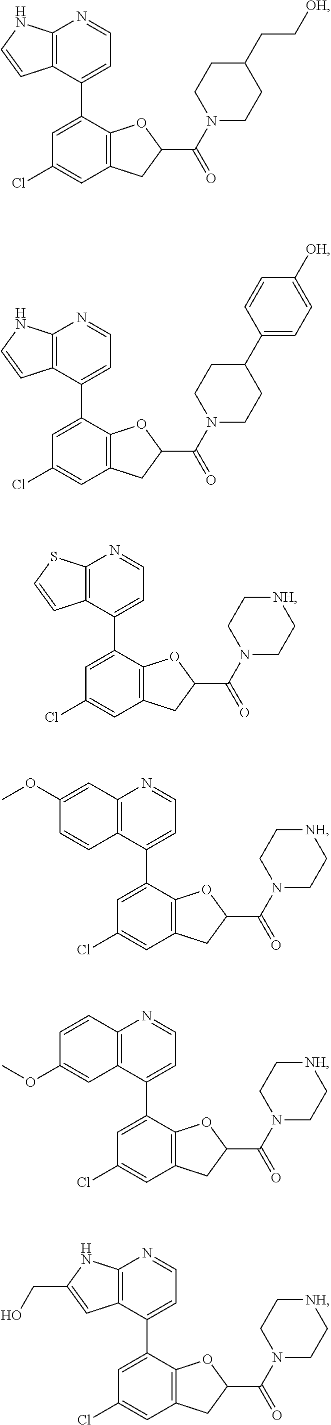 [SMILES: O=C(C1Cc2cc(Cl)cc(-c3ccnc4[nH]ccc34)c2O1)N1CCC(CCO)CC1 ; O=C(C1Cc2cc(Cl)cc(-c3ccnc4[nH]ccc34)c2O1)N1CCC(c2ccc(O)cc2)CC1 ; O=C(C1Cc2cc(Cl)cc(-c3ccnc4sccc34)c2O1)N1CCNCC1 ; COc1ccc2c(-c3cc(Cl)cc4c3OC(C(=O)N3CCNCC3)C4)ccnc2c1 ; COc1ccc2nccc(-c3cc(Cl)cc4c3OC(C(=O)N3CCNCC3)C4)c2c1 ; O=C(C1Cc2cc(Cl)cc(-c3ccnc4[nH]c(CO)cc34)c2O1)N1CCNCC1]

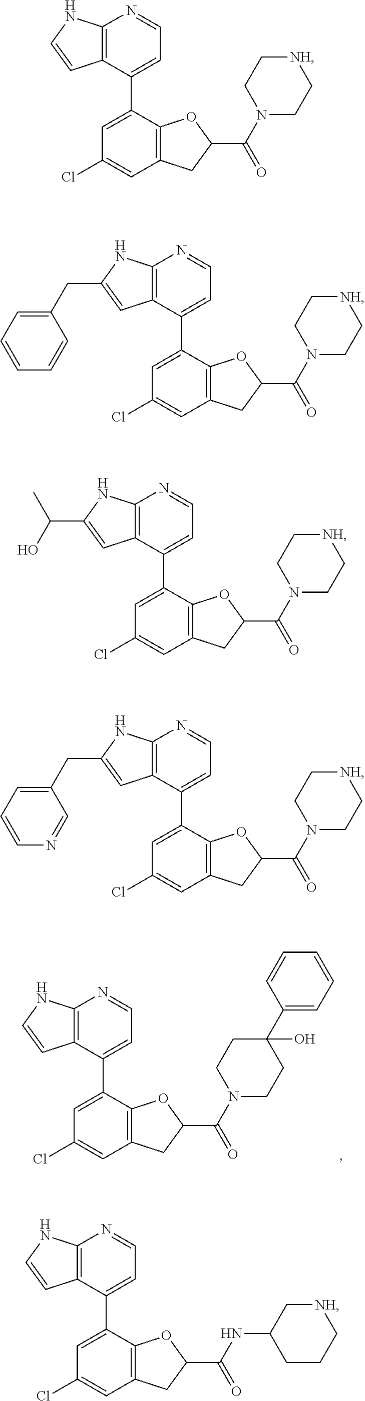 [SMILES: O=C(C1Cc2cc(Cl)cc(-c3ccnc4[nH]ccc34)c2O1)N1CCNCC1 ; O=C(C1Cc2cc(Cl)cc(-c3ccnc4[nH]c(Cc5ccccc5)cc34)c2O1)N1CCNCC1 ; CC(O)c1cc2c(-c3cc(Cl)cc4c3OC(C(=O)N3CCNCC3)C4)ccnc2[nH]1 ; O=C(C1Cc2cc(Cl)cc(-c3ccnc4[nH]c(Cc5cccnc5)cc34)c2O1)N1CCNCC1 ; O=C(C1Cc2cc(Cl)cc(-c3ccnc4[nH]ccc34)c2O1)N1CCC(O)(c2ccccc2)CC1 ; O=C(NC1CCCNC1)C1Cc2cc(Cl)cc(-c3ccnc4[nH]ccc34)c2O1]

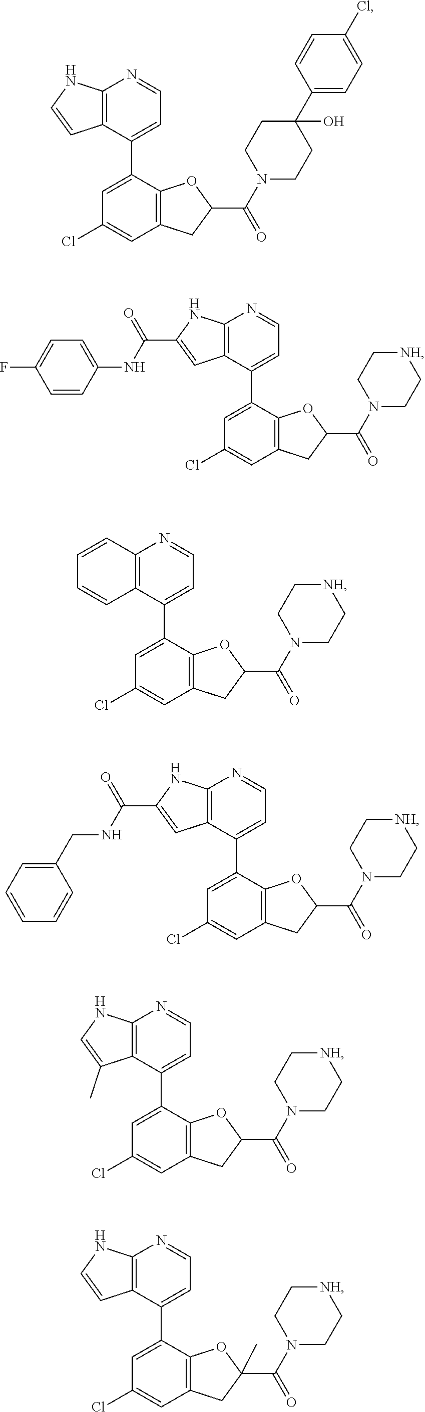 [SMILES: O=C(C1Cc2cc(Cl)cc(-c3ccnc4[nH]ccc34)c2O1)N1CCC(O)(c2ccc(Cl)cc2)CC1 ; O=C(Nc1ccc(F)cc1)c1cc2c(-c3cc(Cl)cc4c3OC(C(=O)N3CCNCC3)C4)ccnc2[nH]1 ; O=C(C1Cc2cc(Cl)cc(-c3ccnc4ccccc34)c2O1)N1CCNCC1 ; O=C(NCc1ccccc1)c1cc2c(-c3cc(Cl)cc4c3OC(C(=O)N3CCNCC3)C4)ccnc2[nH]1 ; Cc1c[nH]c2nccc(-c3cc(Cl)cc4c3OC(C(=O)N3CCNCC3)C4)c12 ; CC1(C(=O)N2CCNCC2)Cc2cc(Cl)cc(-c3ccnc4[nH]ccc34)c2O1]

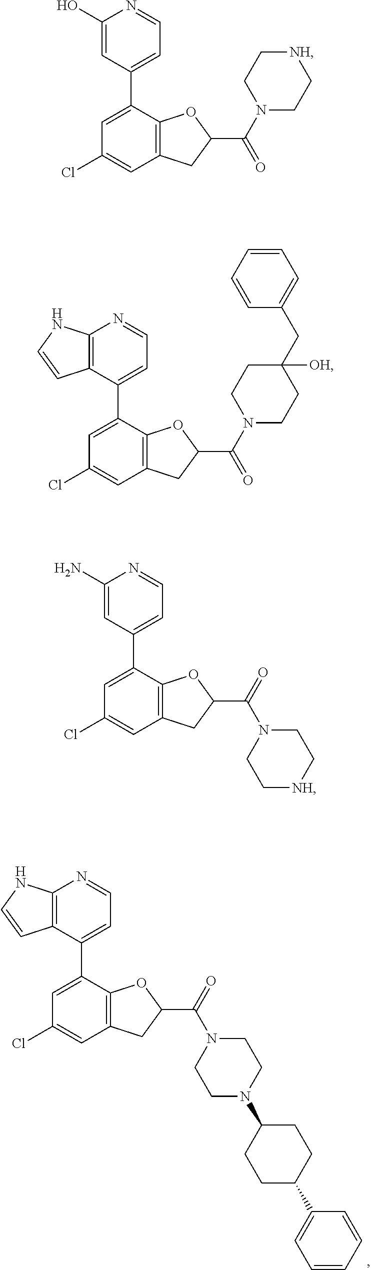 [SMILES: O=C(C1Cc2cc(Cl)cc(-c3ccnc(O)c3)c2O1)N1CCNCC1 ; O=C(C1Cc2cc(Cl)cc(-c3ccnc4[nH]ccc34)c2O1)N1CCC(O)(Cc2ccccc2)CC1 ; Nc1cc(-c2cc(Cl)cc3c2OC(C(=O)N2CCNCC2)C3)ccn1 ; O=C(C1Cc2cc(Cl)cc(-c3ccnc4[nH]ccc34)c2O1)N1CCN([C@H]2CC[C@H](c3ccccc3)CC2)CC1]

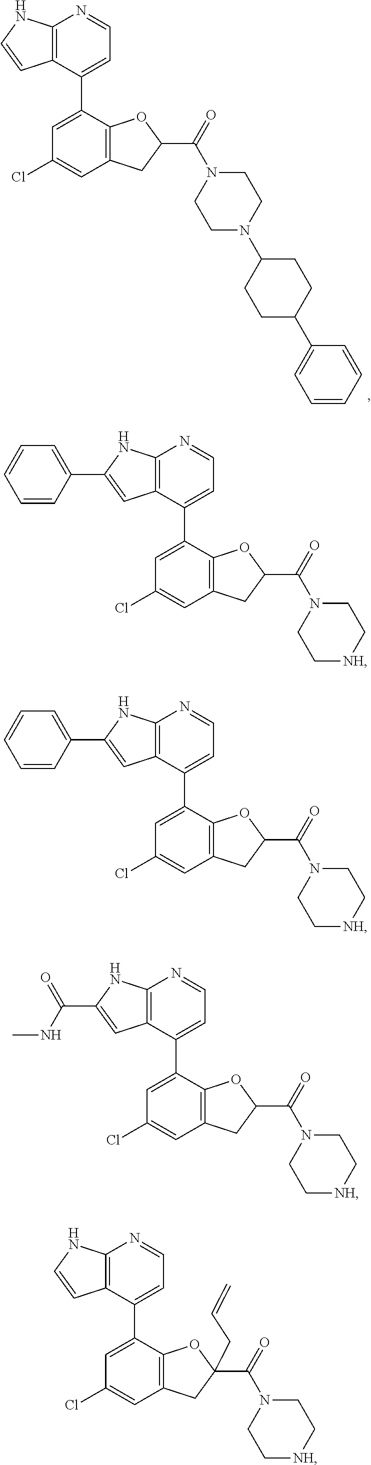 [SMILES: O=C(C1Cc2cc(Cl)cc(-c3ccnc4[nH]ccc34)c2O1)N1CCN(C2CCC(c3ccccc3)CC2)CC1 ; O=C(C1Cc2cc(Cl)cc(-c3ccnc4[nH]c(-c5ccccc5)cc34)c2O1)N1CCNCC1 ; O=C(C1Cc2cc(Cl)cc(-c3ccnc4[nH]c(-c5ccccc5)cc34)c2O1)N1CCNCC1 ; CNC(=O)c1cc2c(-c3cc(Cl)cc4c3OC(C(=O)N3CCNCC3)C4)ccnc2[nH]1 ; C=CCC1(C(=O)N2CCNCC2)Cc2cc(Cl)cc(-c3ccnc4[nH]ccc34)c2O1]

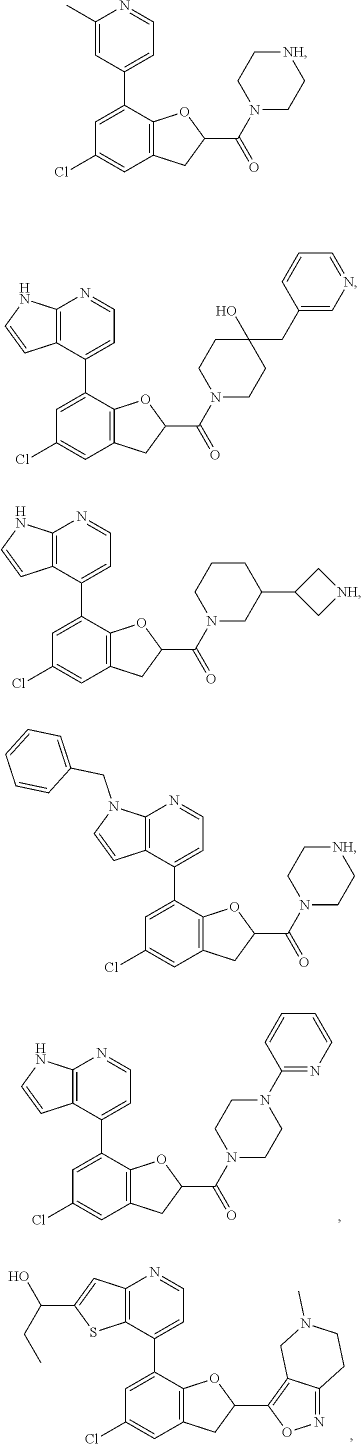

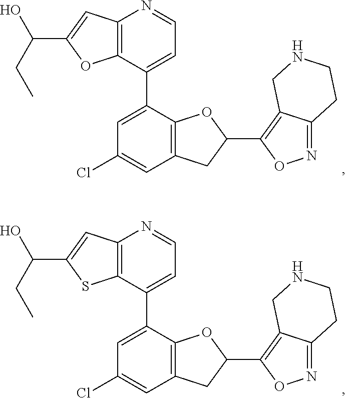 [SMILES: CCC(O)c1cc2nccc(-c3cc(Cl)cc4c3OC(c3onc5c3CNCC5)C4)c2o1 ; CCC(O)c1cc2nccc(-c3cc(Cl)cc4c3OC(c3onc5c3CNCC5)C4)c2s1]

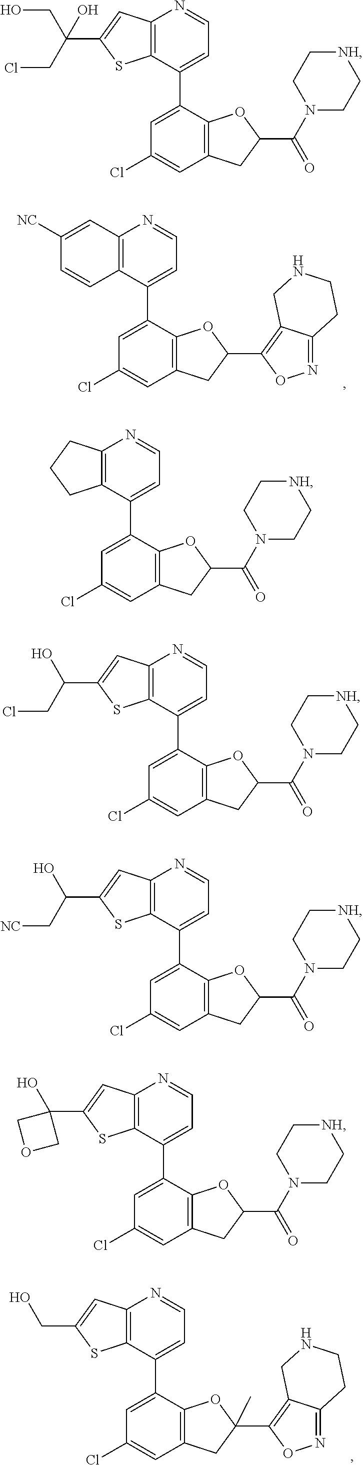

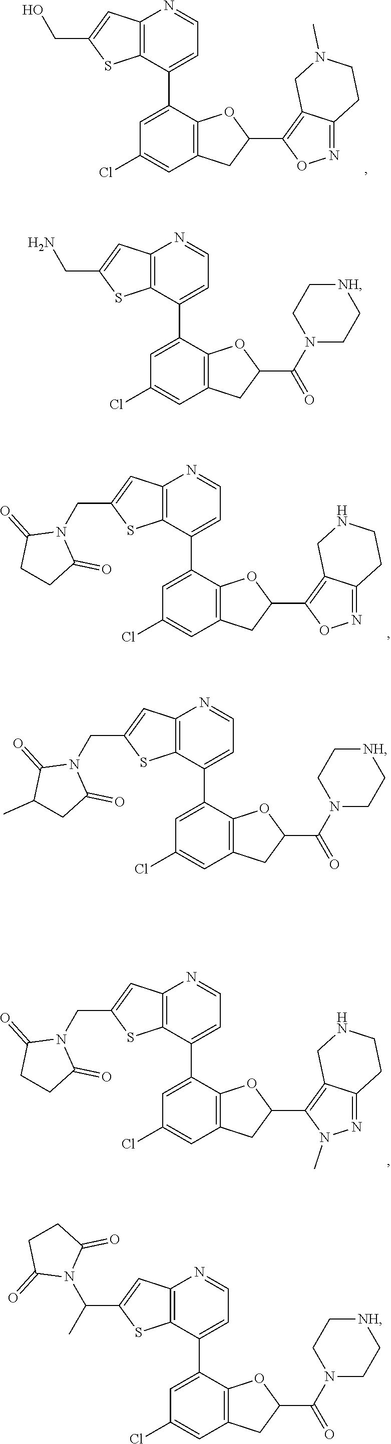 [SMILES: CN1CCc2noc(C3Cc4cc(Cl)cc(-c5ccnc6cc(CO)sc56)c4O3)c2C1 ; NCc1cc2nccc(-c3cc(Cl)cc4c3OC(C(=O)N3CCNCC3)C4)c2s1 ; O=C1CCC(=O)N1Cc1cc2nccc(-c3cc(Cl)cc4c3OC(c3onc5c3CNCC5)C4)c2s1 ; CC1CC(=O)N(Cc2cc3nccc(-c4cc(Cl)cc5c4OC(C(=O)N4CCNCC4)C5)c3s2)C1=O ; Cn1nc2c(c1C1Cc3cc(Cl)cc(-c4ccnc5cc(CN6C(=O)CCC6=O)sc45)c3O1)CNCC2 ; CC(c1cc2nccc(-c3cc(Cl)cc4c3OC(C(=O)N3CCNCC3)C4)c2s1)N1C(=O)CCC1=O]

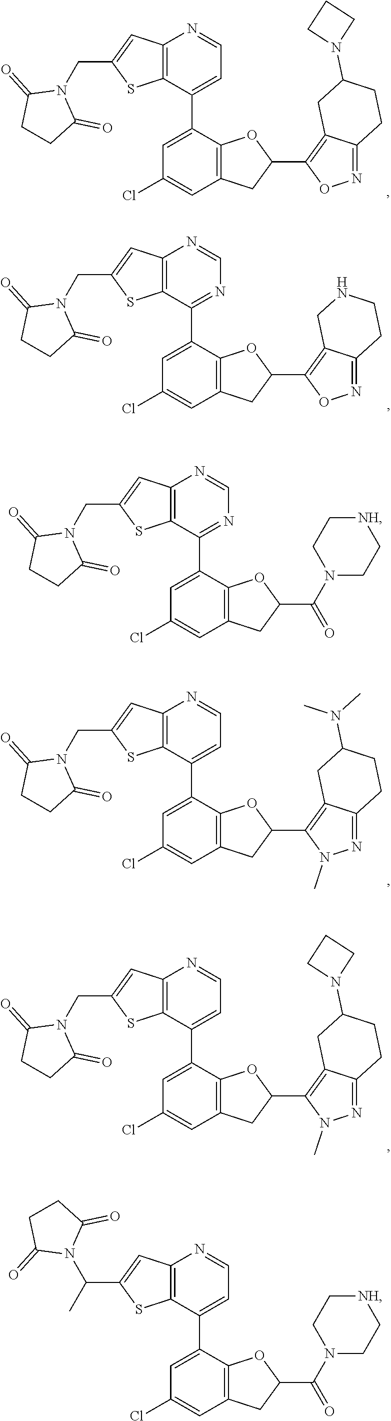

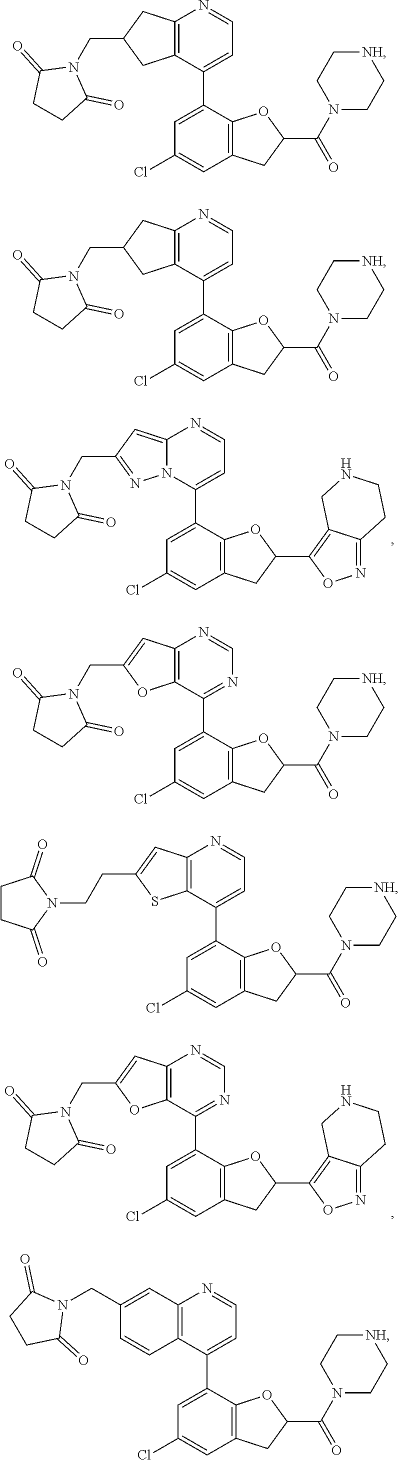

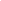

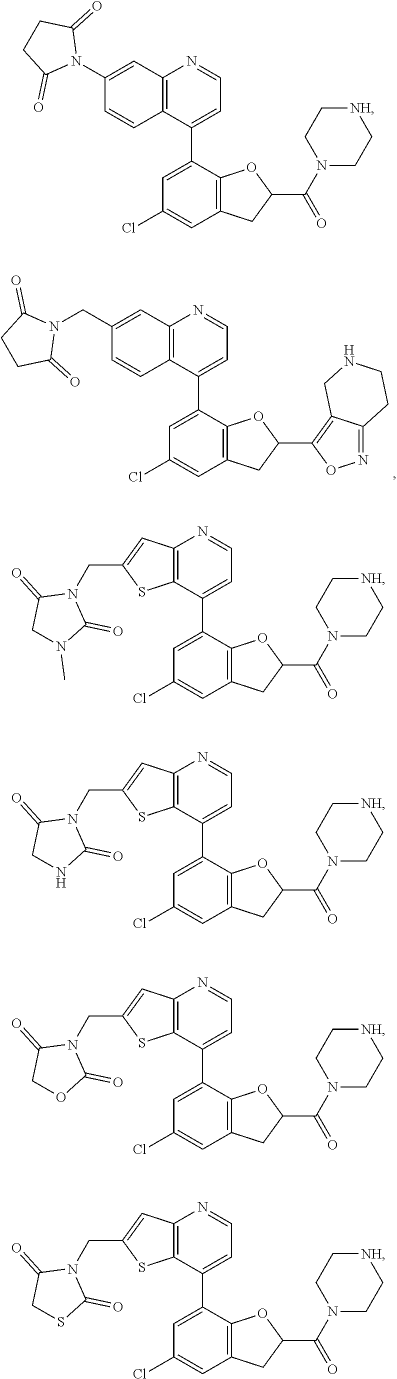 [SMILES: O=C(C1Cc2cc(Cl)cc(-c3ccnc4cc(N5C(=O)CCC5=O)ccc34)c2O1)N1CCNCC1 ; O=C1CCC(=O)N1Cc1ccc2c(-c3cc(Cl)cc4c3OC(c3onc5c3CNCC5)C4)ccnc2c1 ; CN1CC(=O)N(Cc2cc3nccc(-c4cc(Cl)cc5c4OC(C(=O)N4CCNCC4)C5)c3s2)C1=O ; O=C(C1Cc2cc(Cl)cc(-c3ccnc4cc(CN5C(=O)CNC5=O)sc34)c2O1)N1CCNCC1 ; O=C(C1Cc2cc(Cl)cc(-c3ccnc4cc(CN5C(=O)COC5=O)sc34)c2O1)N1CCNCC1 ; O=C(C1Cc2cc(Cl)cc(-c3ccnc4cc(CN5C(=O)CSC5=O)sc34)c2O1)N1CCNCC1]

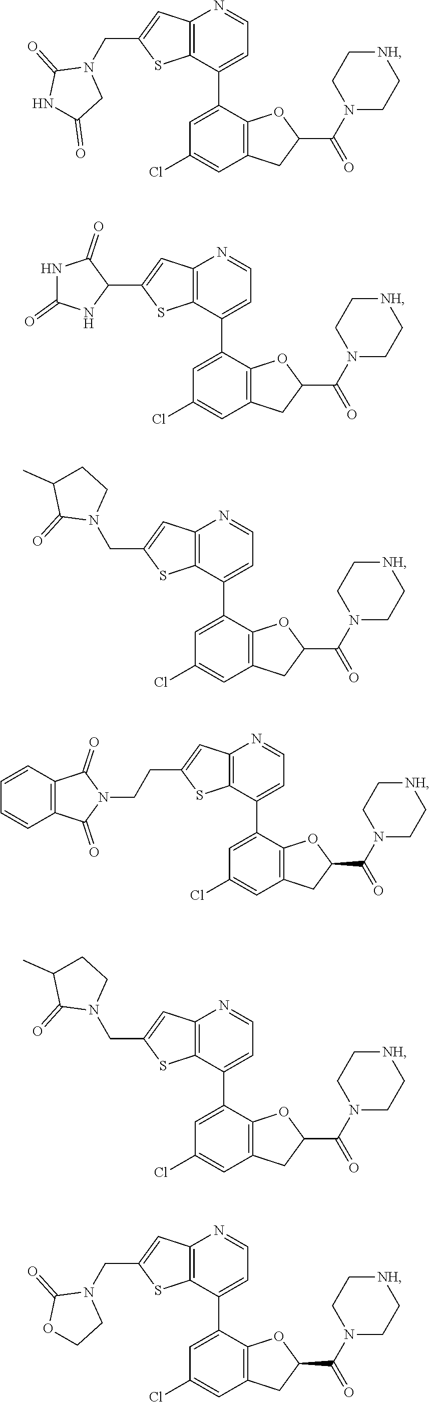 [SMILES: O=C1CN(Cc2cc3nccc(-c4cc(Cl)cc5c4OC(C(=O)N4CCNCC4)C5)c3s2)C(=O)N1 ; O=C1NC(=O)C(c2cc3nccc(-c4cc(Cl)cc5c4OC(C(=O)N4CCNCC4)C5)c3s2)N1 ; CC1CCN(Cc2cc3nccc(-c4cc(Cl)cc5c4OC(C(=O)N4CCNCC4)C5)c3s2)C1=O ; O=C([C@H]1Cc2cc(Cl)cc(-c3ccnc4cc(CCN5C(=O)c6ccccc6C5=O)sc34)c2O1)N1CCNCC1 ; CC1CCN(Cc2cc3nccc(-c4cc(Cl)cc5c4OC(C(=O)N4CCNCC4)C5)c3s2)C1=O ; O=C1OCCN1Cc1cc2nccc(-c3cc(Cl)cc4c3O[C@@H](C(=O)N3CCNCC3)C4)c2s1]

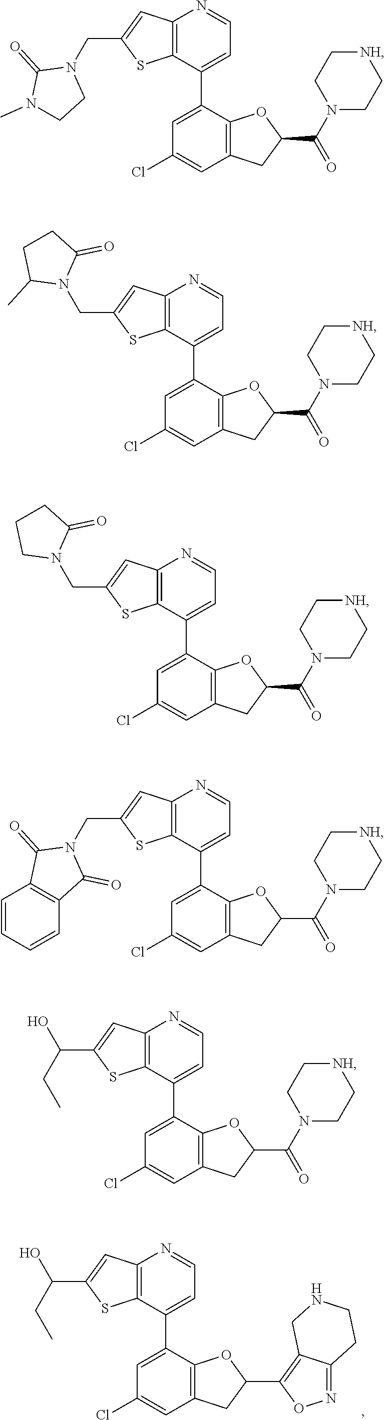 [SMILES: CN1CCN(Cc2cc3nccc(-c4cc(Cl)cc5c4O[C@@H](C(=O)N4CCNCC4)C5)c3s2)C1=O ; CC1CCC(=O)N1Cc1cc2nccc(-c3cc(Cl)cc4c3O[C@@H](C(=O)N3CCNCC3)C4)c2s1 ; O=C1CCCN1Cc1cc2nccc(-c3cc(Cl)cc4c3O[C@@H](C(=O)N3CCNCC3)C4)c2s1 ; O=C(C1Cc2cc(Cl)cc(-c3ccnc4cc(CN5C(=O)c6ccccc6C5=O)sc34)c2O1)N1CCNCC1 ; CCC(O)c1cc2nccc(-c3cc(Cl)cc4c3OC(C(=O)N3CCNCC3)C4)c2s1 ; CCC(O)c1cc2nccc(-c3cc(Cl)cc4c3OC(c3onc5c3CNCC5)C4)c2s1]

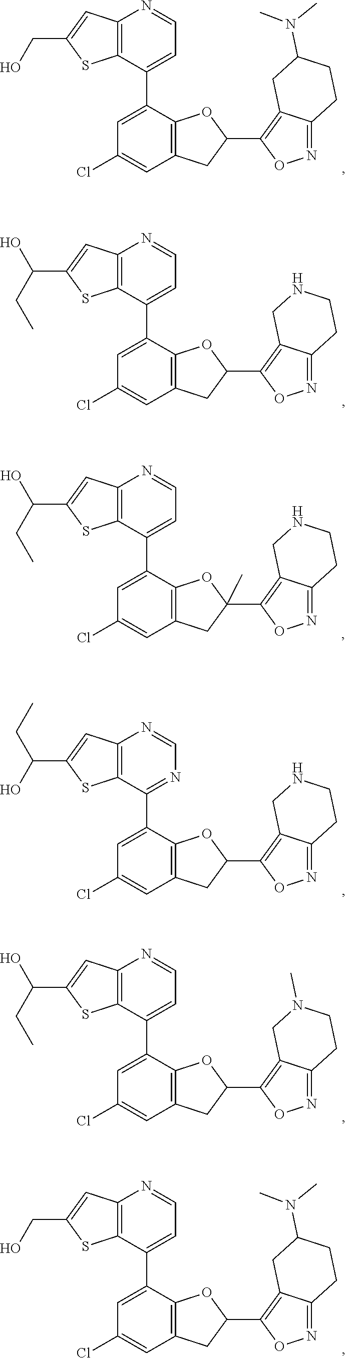 [SMILES: CN(C)C1CCc2noc(C3Cc4cc(Cl)cc(-c5ccnc6cc(CO)sc56)c4O3)c2C1 ; CCC(O)c1cc2nccc(-c3cc(Cl)cc4c3OC(c3onc5c3CNCC5)C4)c2s1 ; CCC(O)c1cc2nccc(-c3cc(Cl)cc4c3OC(C)(c3onc5c3CNCC5)C4)c2s1 ; CCC(O)c1cc2ncnc(-c3cc(Cl)cc4c3OC(c3onc5c3CNCC5)C4)c2s1 ; CCC(O)c1cc2nccc(-c3cc(Cl)cc4c3OC(c3onc5c3CN(C)CC5)C4)c2s1 ; CN(C)C1CCc2noc(C3Cc4cc(Cl)cc(-c5ccnc6cc(CO)sc56)c4O3)c2C1]

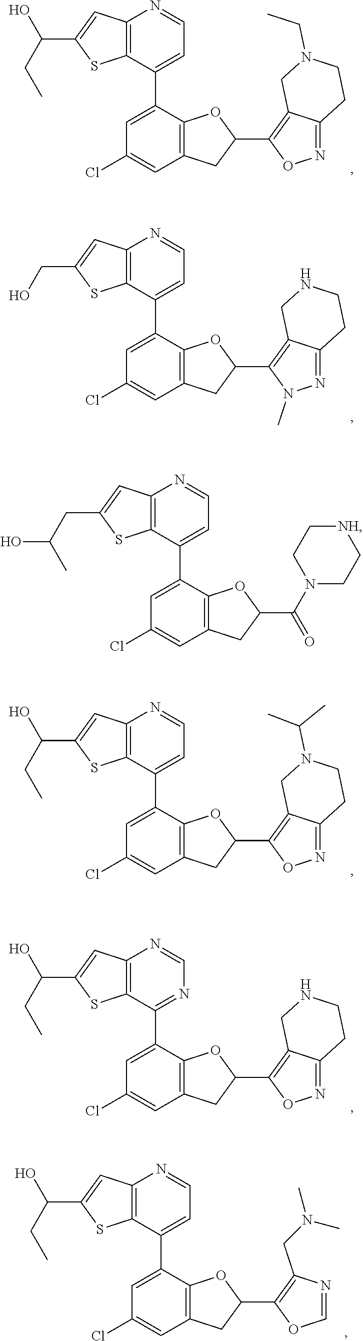 [SMILES: CCC(O)c1cc2nccc(-c3cc(Cl)cc4c3OC(c3onc5c3CN(CC)CC5)C4)c2s1 ; Cn1nc2c(c1C1Cc3cc(Cl)cc(-c4ccnc5cc(CO)sc45)c3O1)CNCC2 ; CC(O)Cc1cc2nccc(-c3cc(Cl)cc4c3OC(C(=O)N3CCNCC3)C4)c2s1 ; CCC(O)c1cc2nccc(-c3cc(Cl)cc4c3OC(c3onc5c3CN(C(C)C)CC5)C4)c2s1 ; CCC(O)c1cc2ncnc(-c3cc(Cl)cc4c3OC(c3onc5c3CNCC5)C4)c2s1 ; CCC(O)c1cc2nccc(-c3cc(Cl)cc4c3OC(c3ocnc3CN(C)C)C4)c2s1]

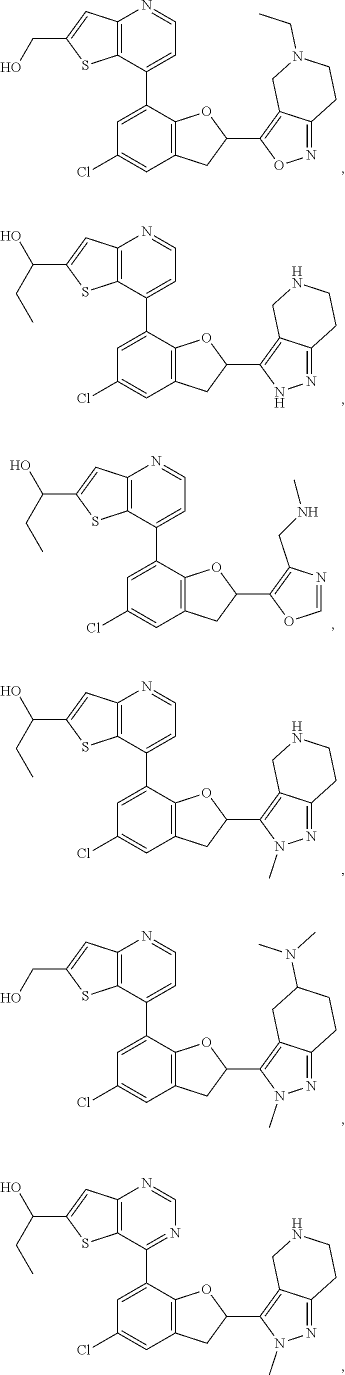 [SMILES: CCN1CCc2noc(C3Cc4cc(Cl)cc(-c5ccnc6cc(CO)sc56)c4O3)c2C1 ; CCC(O)c1cc2nccc(-c3cc(Cl)cc4c3OC(c3[nH]nc5c3CNCC5)C4)c2s1 ; CCC(O)c1cc2nccc(-c3cc(Cl)cc4c3OC(c3ocnc3CNC)C4)c2s1 ; CCC(O)c1cc2nccc(-c3cc(Cl)cc4c3OC(c3c5c(nn3C)CCNC5)C4)c2s1 ; CN(C)C1CCc2nn(C)c(C3Cc4cc(Cl)cc(-c5ccnc6cc(CO)sc56)c4O3)c2C1 ; CCC(O)c1cc2ncnc(-c3cc(Cl)cc4c3OC(c3c5c(nn3C)CCNC5)C4)c2s1]

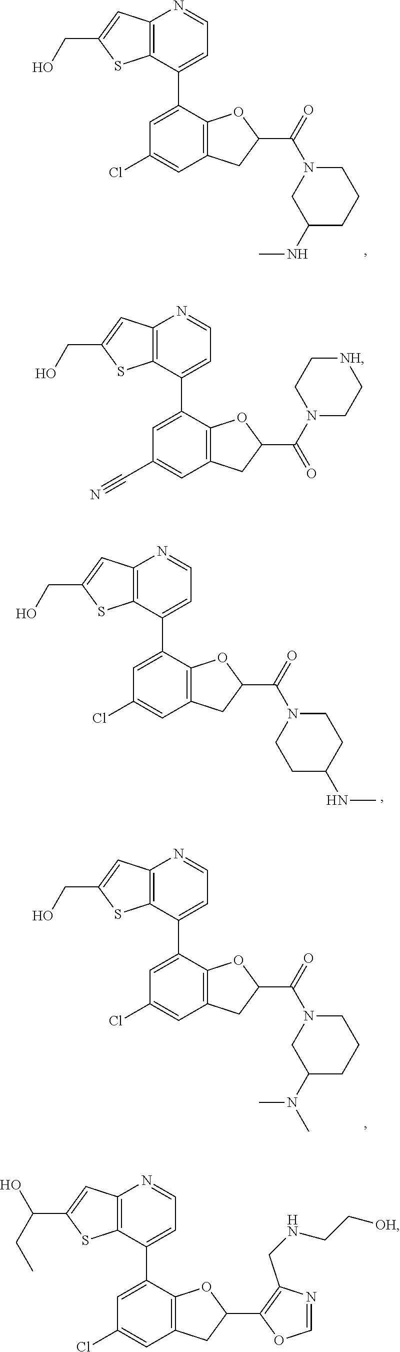 [SMILES: CNC1CCCN(C(=O)C2Cc3cc(Cl)cc(-c4ccnc5cc(CO)sc45)c3O2)C1 ; N#Cc1cc2c(c(-c3ccnc4cc(CO)sc34)c1)OC(C(=O)N1CCNCC1)C2 ; CNC1CCN(C(=O)C2Cc3cc(Cl)cc(-c4ccnc5cc(CO)sc45)c3O2)CC1 ; CN(C)C1CCCN(C(=O)C2Cc3cc(Cl)cc(-c4ccnc5cc(CO)sc45)c3O2)C1 ; CCC(O)c1cc2nccc(-c3cc(Cl)cc4c3OC(c3ocnc3CNCCO)C4)c2s1]

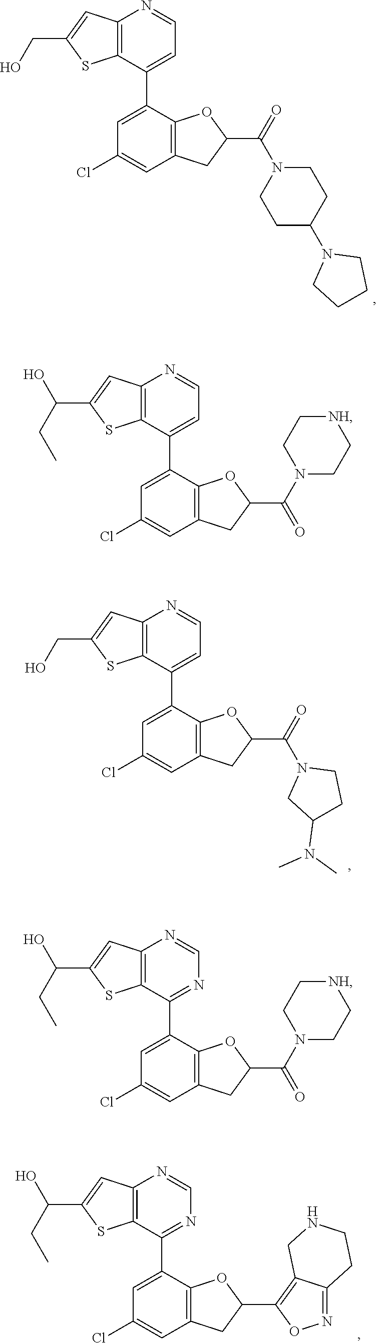 [SMILES: O=C(C1Cc2cc(Cl)cc(-c3ccnc4cc(CO)sc34)c2O1)N1CCC(N2CCCC2)CC1 ; CCC(O)c1cc2nccc(-c3cc(Cl)cc4c3OC(C(=O)N3CCNCC3)C4)c2s1 ; CN(C)C1CCN(C(=O)C2Cc3cc(Cl)cc(-c4ccnc5cc(CO)sc45)c3O2)C1 ; CCC(O)c1cc2ncnc(-c3cc(Cl)cc4c3OC(C(=O)N3CCNCC3)C4)c2s1 ; CCC(O)c1cc2ncnc(-c3cc(Cl)cc4c3OC(c3onc5c3CNCC5)C4)c2s1]

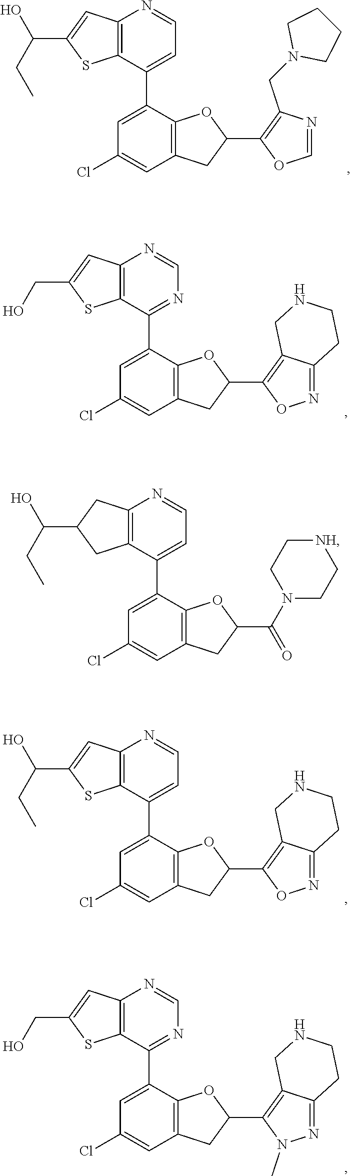 [SMILES: CCC(O)c1cc2nccc(-c3cc(Cl)cc4c3OC(c3ocnc3CN3CCCC3)C4)c2s1 ; OCc1cc2ncnc(-c3cc(Cl)cc4c3OC(c3onc5c3CNCC5)C4)c2s1 ; CCC(O)C1Cc2nccc(-c3cc(Cl)cc4c3OC(C(=O)N3CCNCC3)C4)c2C1 ; CCC(O)c1cc2nccc(-c3cc(Cl)cc4c3OC(c3onc5c3CNCC5)C4)c2s1 ; Cn1nc2c(c1C1Cc3cc(Cl)cc(-c4ncnc5cc(CO)sc45)c3O1)CNCC2]

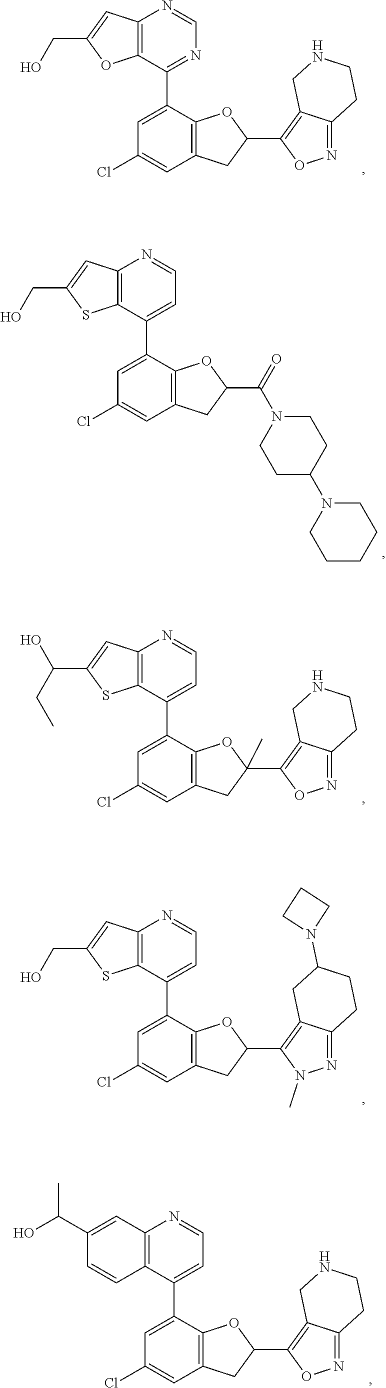 [SMILES: OCc1cc2ncnc(-c3cc(Cl)cc4c3OC(c3onc5c3CNCC5)C4)c2o1 ; O=C(C1Cc2cc(Cl)cc(-c3ccnc4cc(CO)sc34)c2O1)N1CCC(N2CCCCC2)CC1 ; CCC(O)c1cc2nccc(-c3cc(Cl)cc4c3OC(C)(c3onc5c3CNCC5)C4)c2s1 ; Cn1nc2c(c1C1Cc3cc(Cl)cc(-c4ccnc5cc(CO)sc45)c3O1)CC(N1CCC1)CC2 ; CC(O)c1ccc2c(-c3cc(Cl)cc4c3OC(c3onc5c3CNCC5)C4)ccnc2c1]

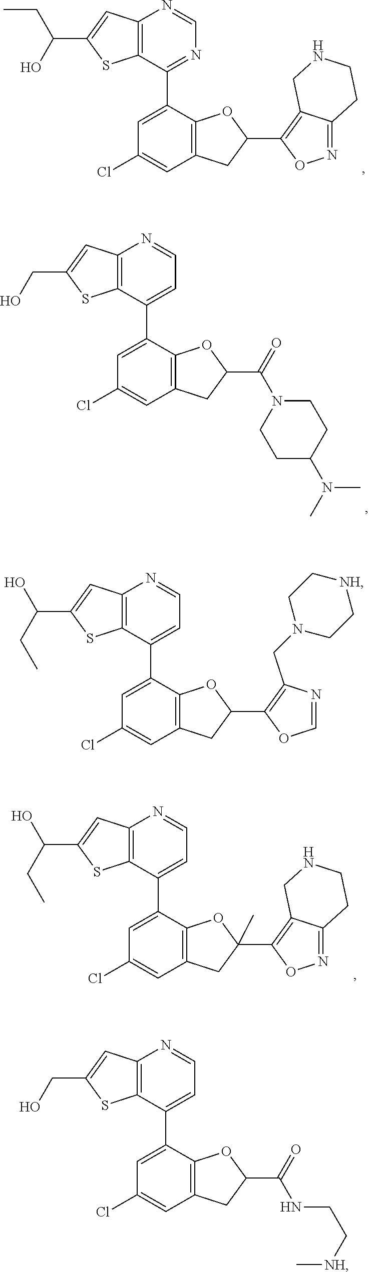 [SMILES: CCC(O)c1cc2ncnc(-c3cc(Cl)cc4c3OC(c3onc5c3CNCC5)C4)c2s1 ; CN(C)C1CCN(C(=O)C2Cc3cc(Cl)cc(-c4ccnc5cc(CO)sc45)c3O2)CC1 ; CCC(O)c1cc2nccc(-c3cc(Cl)cc4c3OC(c3ocnc3CN3CCNCC3)C4)c2s1 ; CCC(O)c1cc2nccc(-c3cc(Cl)cc4c3OC(C)(c3onc5c3CNCC5)C4)c2s1 ; CNCCNC(=O)C1Cc2cc(Cl)cc(-c3ccnc4cc(CO)sc34)c2O1]

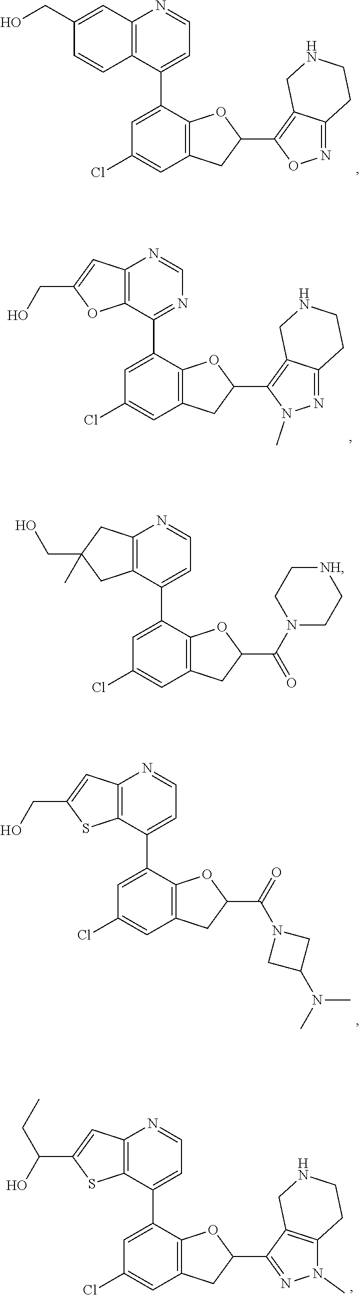 [SMILES: OCc1ccc2c(-c3cc(Cl)cc4c3OC(c3onc5c3CNCC5)C4)ccnc2c1 ; Cn1nc2c(c1C1Cc3cc(Cl)cc(-c4ncnc5cc(CO)oc45)c3O1)CNCC2 ; CC1(CO)Cc2nccc(-c3cc(Cl)cc4c3OC(C(=O)N3CCNCC3)C4)c2C1 ; CN(C)C1CN(C(=O)C2Cc3cc(Cl)cc(-c4ccnc5cc(CO)sc45)c3O2)C1 ; CCC(O)c1cc2nccc(-c3cc(Cl)cc4c3OC(c3nn(C)c5c3CNCC5)C4)c2s1]

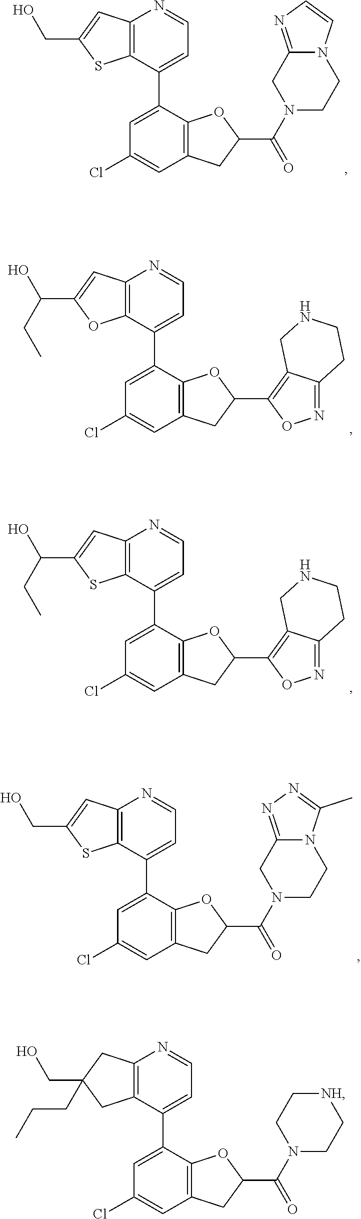

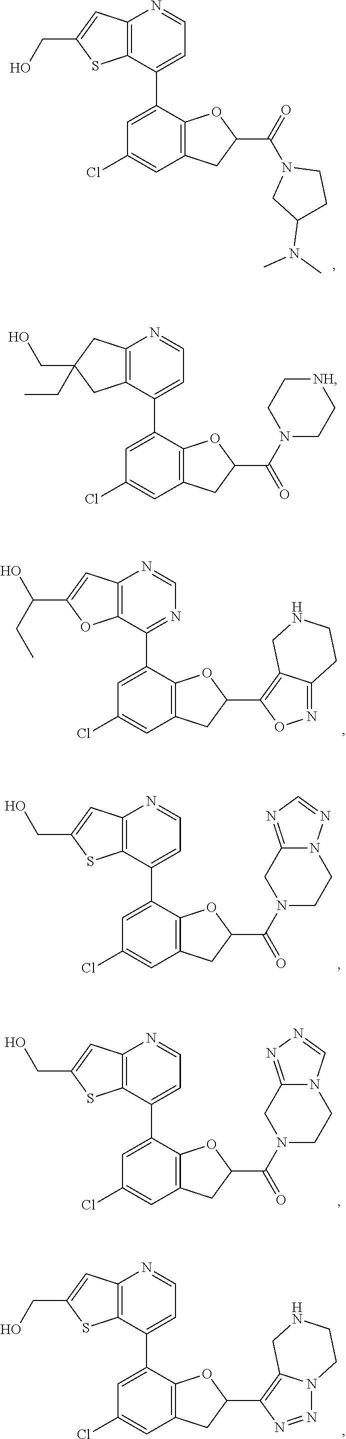 [SMILES: CN(C)C1CCN(C(=O)C2Cc3cc(Cl)cc(-c4ccnc5cc(CO)sc45)c3O2)C1 ; CCC1(CO)Cc2nccc(-c3cc(Cl)cc4c3OC(C(=O)N3CCNCC3)C4)c2C1 ; CCC(O)c1cc2ncnc(-c3cc(Cl)cc4c3OC(c3onc5c3CNCC5)C4)c2o1 ; O=C(C1Cc2cc(Cl)cc(-c3ccnc4cc(CO)sc34)c2O1)N1CCn2ncnc2C1 ; O=C(C1Cc2cc(Cl)cc(-c3ccnc4cc(CO)sc34)c2O1)N1CCn2cnnc2C1 ; OCc1cc2nccc(-c3cc(Cl)cc4c3OC(c3nnn5c3CNCC5)C4)c2s1]

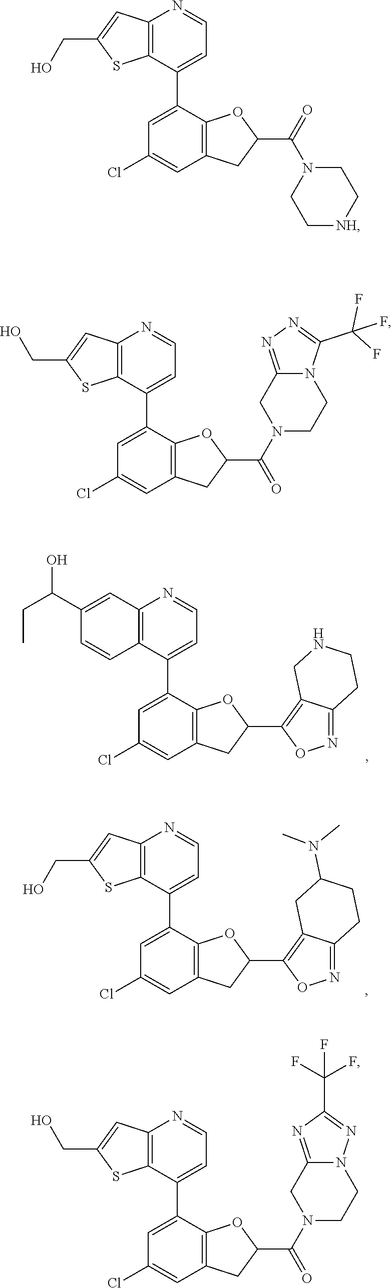 [SMILES: O=C(C1Cc2cc(Cl)cc(-c3ccnc4cc(CO)sc34)c2O1)N1CCNCC1 ; O=C(C1Cc2cc(Cl)cc(-c3ccnc4cc(CO)sc34)c2O1)N1CCn2c(nnc2C(F)(F)F)C1 ; CCC(O)c1ccc2c(-c3cc(Cl)cc4c3OC(c3onc5c3CNCC5)C4)ccnc2c1 ; CN(C)C1CCc2noc(C3Cc4cc(Cl)cc(-c5ccnc6cc(CO)sc56)c4O3)c2C1 ; O=C(C1Cc2cc(Cl)cc(-c3ccnc4cc(CO)sc34)c2O1)N1CCn2nc(C(F)(F)F)nc2C1]

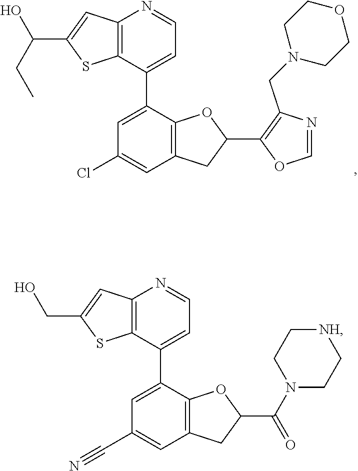 [SMILES: CCC(O)c1cc2nccc(-c3cc(Cl)cc4c3OC(c3ocnc3CN3CCOCC3)C4)c2s1 ; N#Cc1cc2c(c(-c3ccnc4cc(CO)sc34)c1)OC(C(=O)N1CCNCC1)C2]

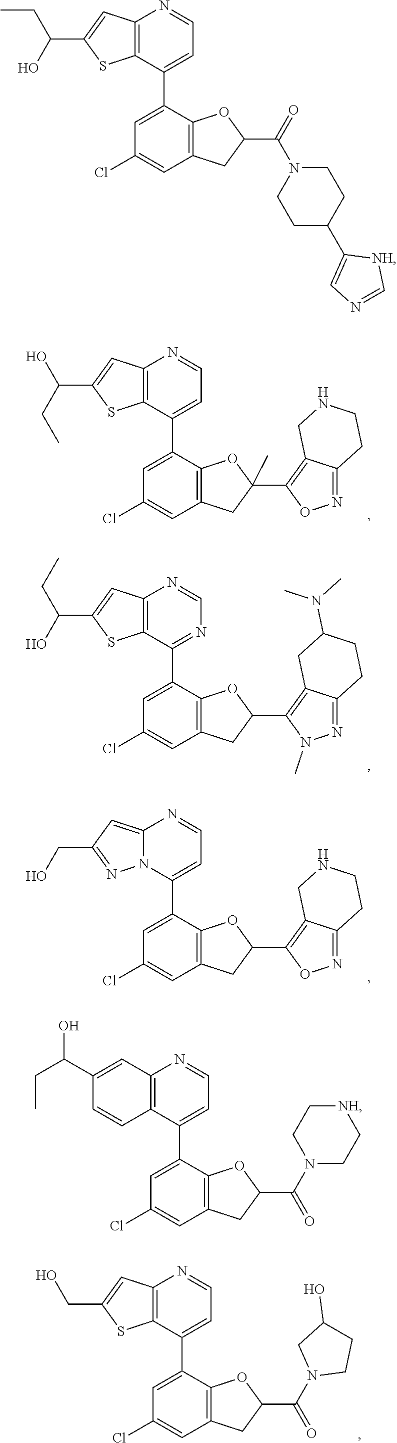

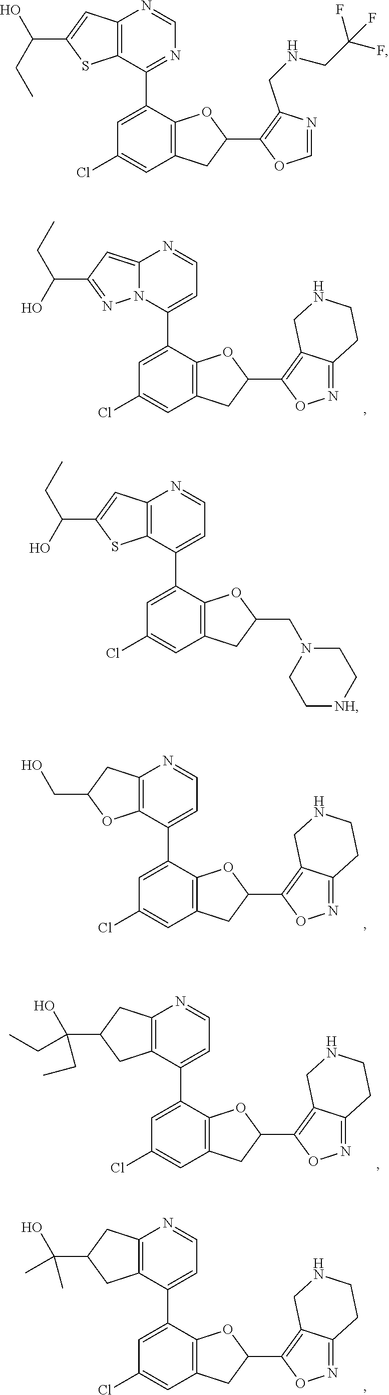 [SMILES: CCC(O)c1cc2ncnc(-c3cc(Cl)cc4c3OC(c3ocnc3CNCC(F)(F)F)C4)c2s1 ; CCC(O)c1cc2nccc(-c3cc(Cl)cc4c3OC(c3onc5c3CNCC5)C4)n2n1 ; CCC(O)c1cc2nccc(-c3cc(Cl)cc4c3OC(CN3CCNCC3)C4)c2s1 ; OCC1Cc2nccc(-c3cc(Cl)cc4c3OC(c3onc5c3CNCC5)C4)c2O1 ; CCC(O)(CC)C1Cc2nccc(-c3cc(Cl)cc4c3OC(c3onc5c3CNCC5)C4)c2C1 ; CC(C)(O)C1Cc2nccc(-c3cc(Cl)cc4c3OC(c3onc5c3CNCC5)C4)c2C1]

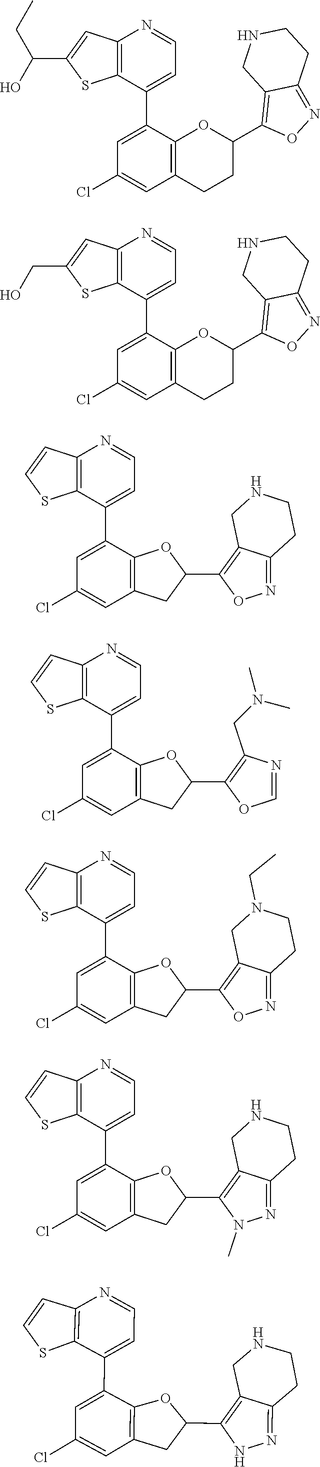

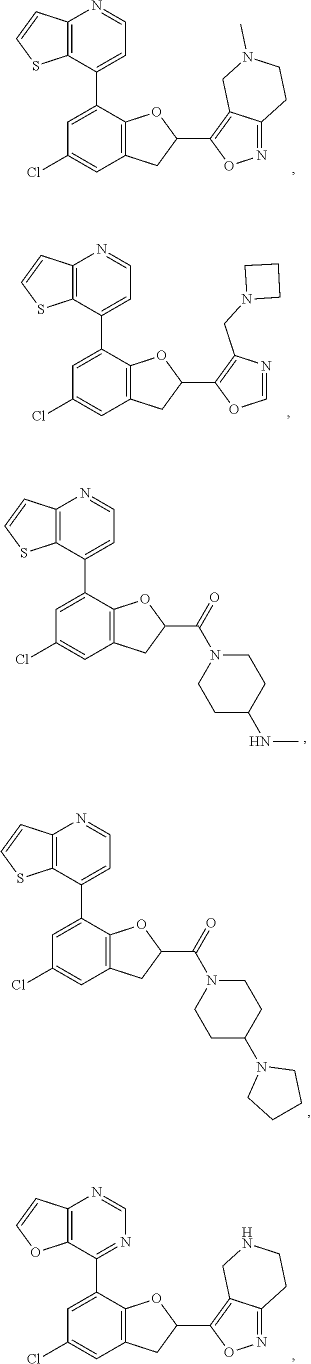 [SMILES: CN1CCc2noc(C3Cc4cc(Cl)cc(-c5ccnc6ccsc56)c4O3)c2C1 ; Clc1cc2c(c(-c3ccnc4ccsc34)c1)OC(c1ocnc1CN1CCC1)C2 ; CNC1CCN(C(=O)C2Cc3cc(Cl)cc(-c4ccnc5ccsc45)c3O2)CC1 ; O=C(C1Cc2cc(Cl)cc(-c3ccnc4ccsc34)c2O1)N1CCC(N2CCCC2)CC1 ; Clc1cc2c(c(-c3ncnc4ccoc34)c1)OC(c1onc3c1CNCC3)C2]

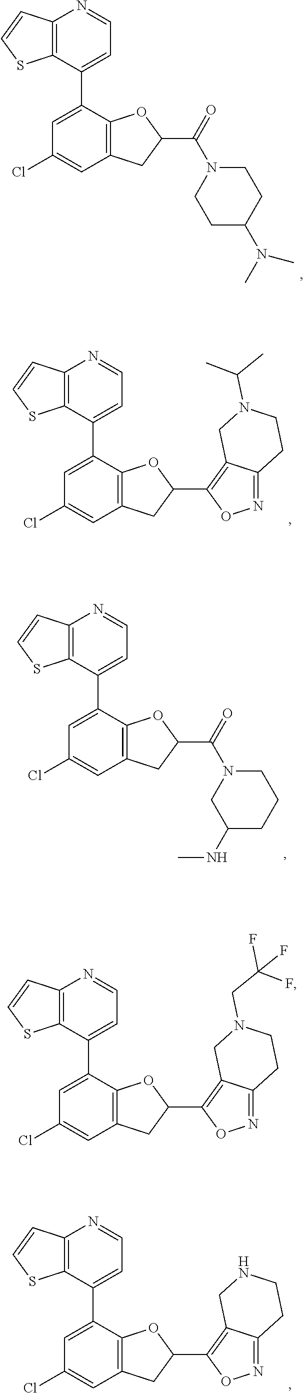 [SMILES: CN(C)C1CCN(C(=O)C2Cc3cc(Cl)cc(-c4ccnc5ccsc45)c3O2)CC1 ; CC(C)N1CCc2noc(C3Cc4cc(Cl)cc(-c5ccnc6ccsc56)c4O3)c2C1 ; CNC1CCCN(C(=O)C2Cc3cc(Cl)cc(-c4ccnc5ccsc45)c3O2)C1 ; FC(F)(F)CN1CCc2noc(C3Cc4cc(Cl)cc(-c5ccnc6ccsc56)c4O3)c2C1 ; Clc1cc2c(c(-c3ccnc4ccsc34)c1)OC(c1onc3c1CNCC3)C2]

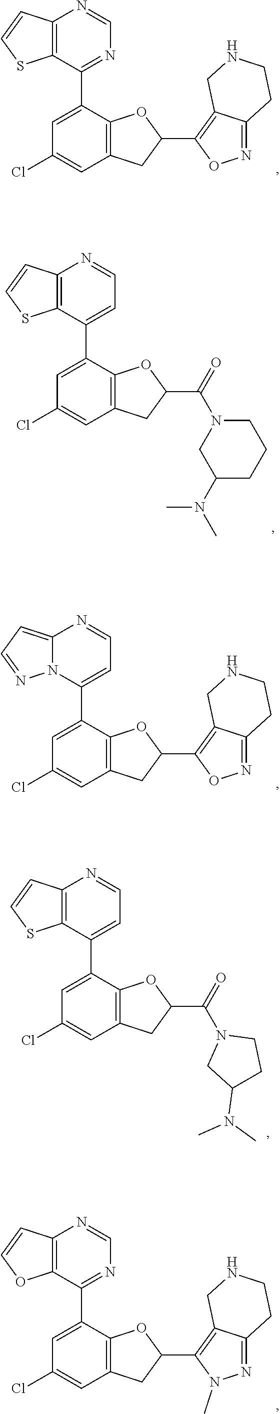 [SMILES: Clc1cc2c(c(-c3ncnc4ccsc34)c1)OC(c1onc3c1CNCC3)C2 ; CN(C)C1CCCN(C(=O)C2Cc3cc(Cl)cc(-c4ccnc5ccsc45)c3O2)C1 ; Clc1cc2c(c(-c3ccnc4ccnn34)c1)OC(c1onc3c1CNCC3)C2 ; CN(C)C1CCN(C(=O)C2Cc3cc(Cl)cc(-c4ccnc5ccsc45)c3O2)C1 ; Cn1nc2c(c1C1Cc3cc(Cl)cc(-c4ncnc5ccoc45)c3O1)CNCC2]

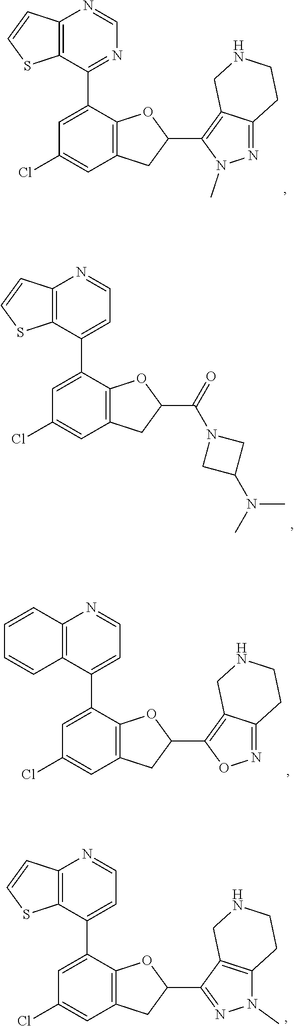 [SMILES: Cn1nc2c(c1C1Cc3cc(Cl)cc(-c4ncnc5ccsc45)c3O1)CNCC2 ; CN(C)C1CN(C(=O)C2Cc3cc(Cl)cc(-c4ccnc5ccsc45)c3O2)C1 ; Clc1cc2c(c(-c3ccnc4ccccc34)c1)OC(c1onc3c1CNCC3)C2 ; Cn1nc(C2Cc3cc(Cl)cc(-c4ccnc5ccsc45)c3O2)c2c1CCNC2]

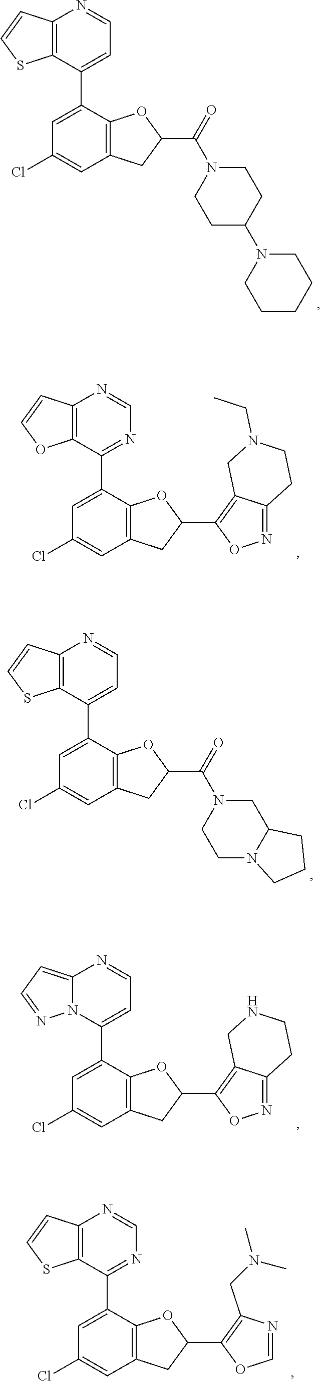 [SMILES: O=C(C1Cc2cc(Cl)cc(-c3ccnc4ccsc34)c2O1)N1CCC(N2CCCCC2)CC1 ; CCN1CCc2noc(C3Cc4cc(Cl)cc(-c5ncnc6ccoc56)c4O3)c2C1 ; O=C(C1Cc2cc(Cl)cc(-c3ccnc4ccsc34)c2O1)N1CCN2CCCC2C1 ; Clc1cc2c(c(-c3ccnc4ccnn34)c1)OC(c1onc3c1CNCC3)C2 ; CN(C)Cc1ncoc1C1Cc2cc(Cl)cc(-c3ncnc4ccsc34)c2O1]

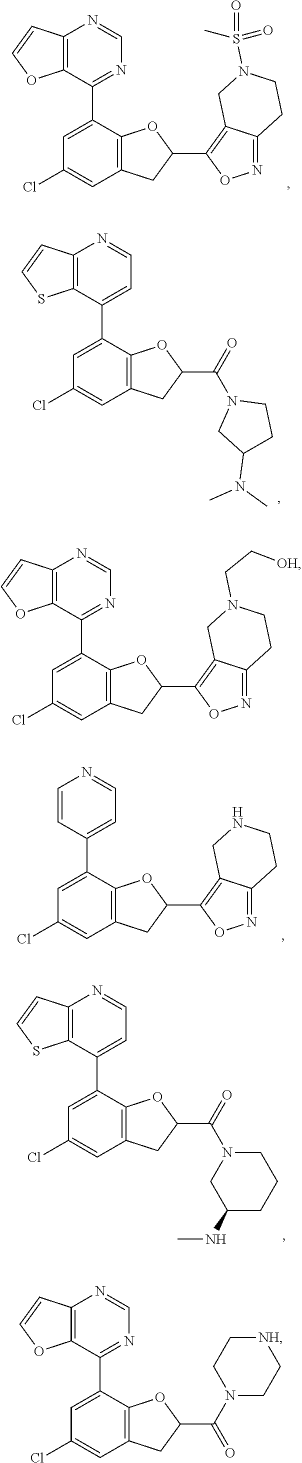 [SMILES: CS(=O)(=O)N1CCc2noc(C3Cc4cc(Cl)cc(-c5ncnc6ccoc56)c4O3)c2C1 ; CN(C)C1CCN(C(=O)C2Cc3cc(Cl)cc(-c4ccnc5ccsc45)c3O2)C1 ; OCCN1CCc2noc(C3Cc4cc(Cl)cc(-c5ncnc6ccoc56)c4O3)c2C1 ; Clc1cc2c(c(-c3ccncc3)c1)OC(c1onc3c1CNCC3)C2 ; CN[C@@H]1CCCN(C(=O)C2Cc3cc(Cl)cc(-c4ccnc5ccsc45)c3O2)C1 ; O=C(C1Cc2cc(Cl)cc(-c3ncnc4ccoc34)c2O1)N1CCNCC1]

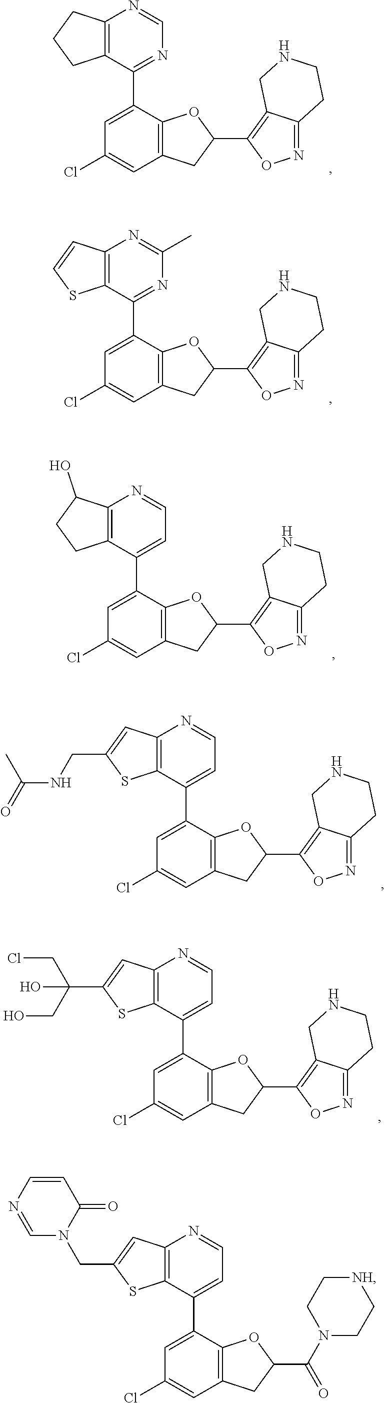 [SMILES: Clc1cc2c(c(-c3ncnc4c3CCC4)c1)OC(c1onc3c1CNCC3)C2 ; Cc1nc(-c2cc(Cl)cc3c2OC(c2onc4c2CNCC4)C3)c2sccc2n1 ; OC1CCc2c(-c3cc(Cl)cc4c3OC(c3onc5c3CNCC5)C4)ccnc21 ; CC(=O)NCc1cc2nccc(-c3cc(Cl)cc4c3OC(c3onc5c3CNCC5)C4)c2s1 ; OCC(O)(CCl)c1cc2nccc(-c3cc(Cl)cc4c3OC(c3onc5c3CNCC5)C4)c2s1 ; O=C(C1Cc2cc(Cl)cc(-c3ccnc4cc(Cn5cnccc5=O)sc34)c2O1)N1CCNCC1]

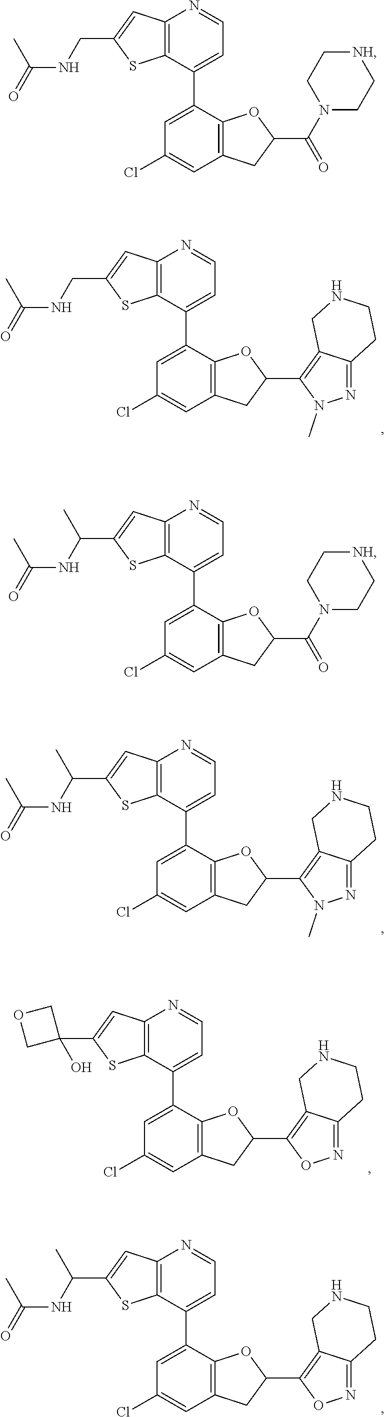 [SMILES: CC(=O)NCc1cc2nccc(-c3cc(Cl)cc4c3OC(C(=O)N3CCNCC3)C4)c2s1 ; CC(=O)NCc1cc2nccc(-c3cc(Cl)cc4c3OC(c3c5c(nn3C)CCNC5)C4)c2s1 ; CC(=O)NC(C)c1cc2nccc(-c3cc(Cl)cc4c3OC(C(=O)N3CCNCC3)C4)c2s1 ; CC(=O)NC(C)c1cc2nccc(-c3cc(Cl)cc4c3OC(c3c5c(nn3C)CCNC5)C4)c2s1 ; OC1(c2cc3nccc(-c4cc(Cl)cc5c4OC(c4onc6c4CNCC6)C5)c3s2)COC1 ; CC(=O)NC(C)c1cc2nccc(-c3cc(Cl)cc4c3OC(c3onc5c3CNCC5)C4)c2s1]

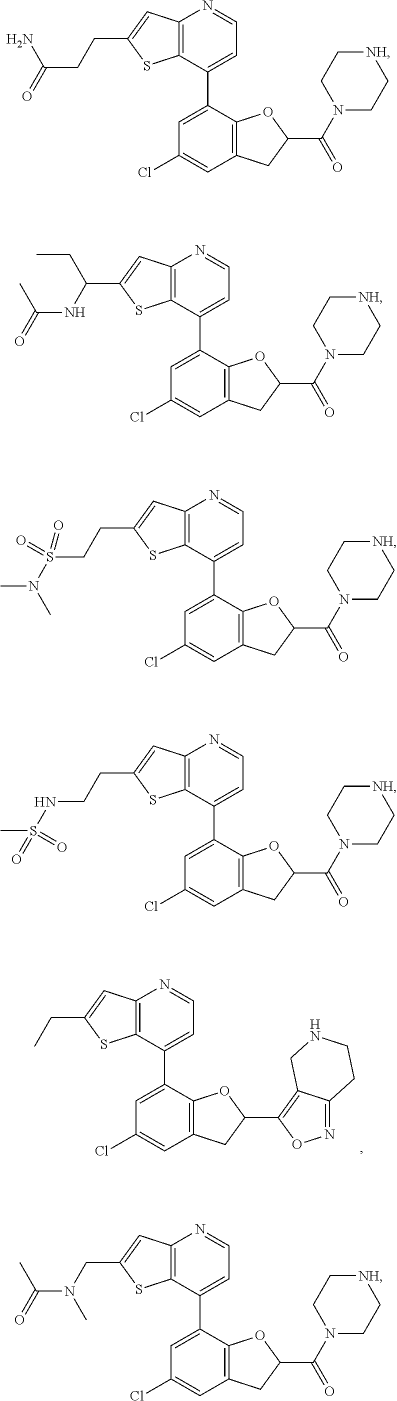 [SMILES: NC(=O)CCc1cc2nccc(-c3cc(Cl)cc4c3OC(C(=O)N3CCNCC3)C4)c2s1 ; CCC(NC(C)=O)c1cc2nccc(-c3cc(Cl)cc4c3OC(C(=O)N3CCNCC3)C4)c2s1 ; CN(C)S(=O)(=O)CCc1cc2nccc(-c3cc(Cl)cc4c3OC(C(=O)N3CCNCC3)C4)c2s1 ; CS(=O)(=O)NCCc1cc2nccc(-c3cc(Cl)cc4c3OC(C(=O)N3CCNCC3)C4)c2s1 ; CCc1cc2nccc(-c3cc(Cl)cc4c3OC(c3onc5c3CNCC5)C4)c2s1 ; CC(=O)N(C)Cc1cc2nccc(-c3cc(Cl)cc4c3OC(C(=O)N3CCNCC3)C4)c2s1]

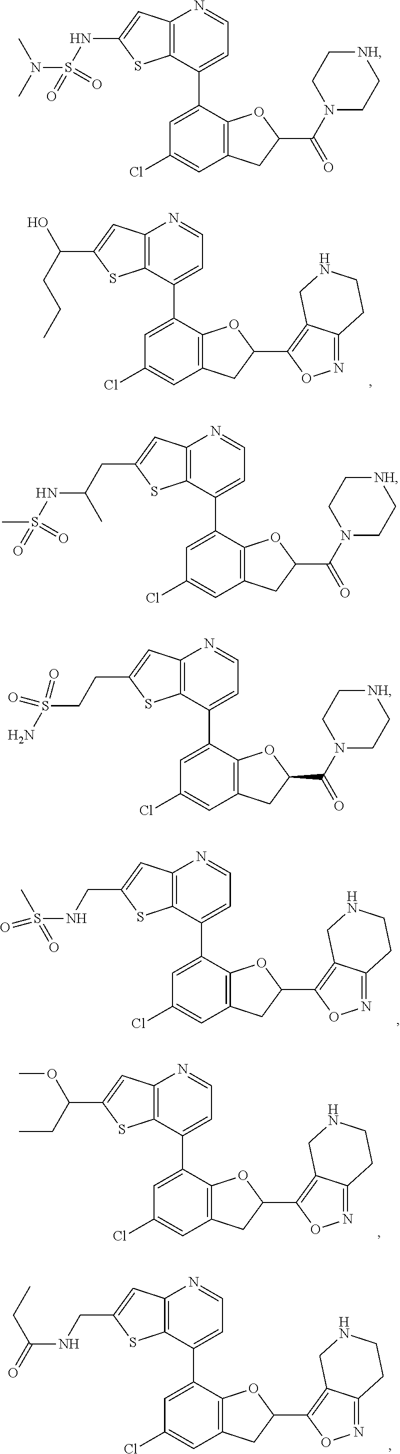

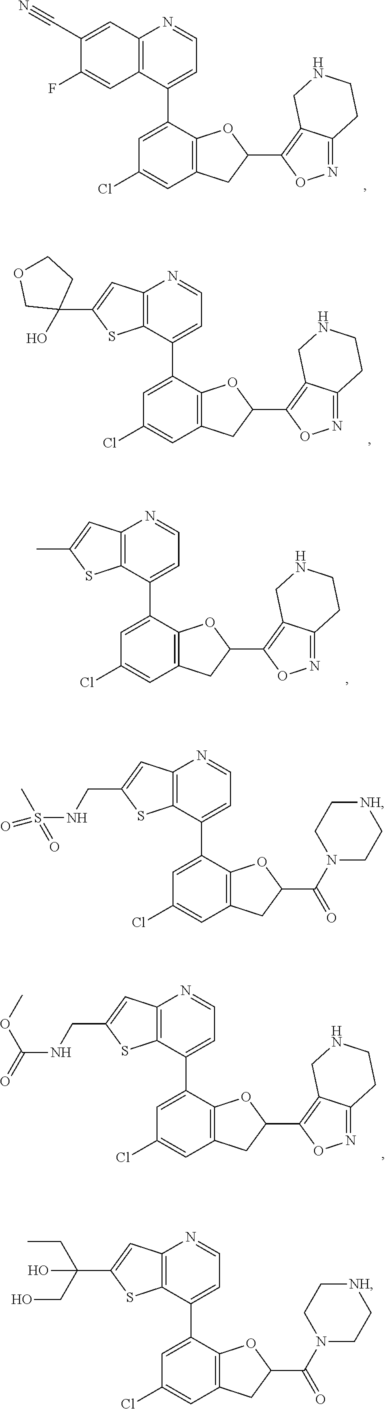 [SMILES: N#Cc1cc2nccc(-c3cc(Cl)cc4c3OC(c3onc5c3CNCC5)C4)c2cc1F ; OC1(c2cc3nccc(-c4cc(Cl)cc5c4OC(c4onc6c4CNCC6)C5)c3s2)CCOC1 ; Cc1cc2nccc(-c3cc(Cl)cc4c3OC(c3onc5c3CNCC5)C4)c2s1 ; CS(=O)(=O)NCc1cc2nccc(-c3cc(Cl)cc4c3OC(C(=O)N3CCNCC3)C4)c2s1 ; COC(=O)NCc1cc2nccc(-c3cc(Cl)cc4c3OC(c3onc5c3CNCC5)C4)c2s1 ; CCC(O)(CO)c1cc2nccc(-c3cc(Cl)cc4c3OC(C(=O)N3CCNCC3)C4)c2s1]

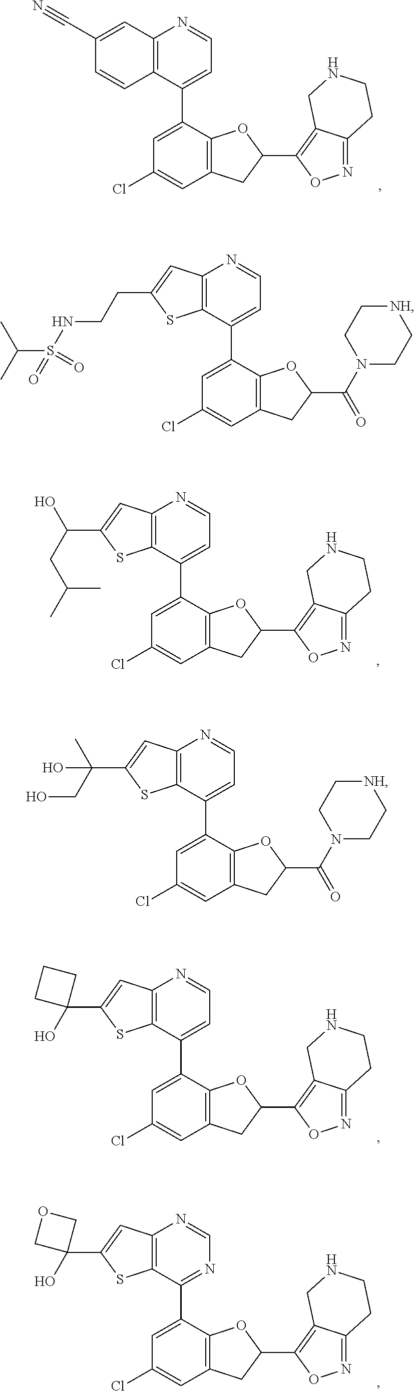 [SMILES: N#Cc1ccc2c(-c3cc(Cl)cc4c3OC(c3onc5c3CNCC5)C4)ccnc2c1 ; CC(C)S(=O)(=O)NCCc1cc2nccc(-c3cc(Cl)cc4c3OC(C(=O)N3CCNCC3)C4)c2s1 ; CC(C)CC(O)c1cc2nccc(-c3cc(Cl)cc4c3OC(c3onc5c3CNCC5)C4)c2s1 ; CC(O)(CO)c1cc2nccc(-c3cc(Cl)cc4c3OC(C(=O)N3CCNCC3)C4)c2s1 ; OC1(c2cc3nccc(-c4cc(Cl)cc5c4OC(c4onc6c4CNCC6)C5)c3s2)CCC1 ; OC1(c2cc3ncnc(-c4cc(Cl)cc5c4OC(c4onc6c4CNCC6)C5)c3s2)COC1]

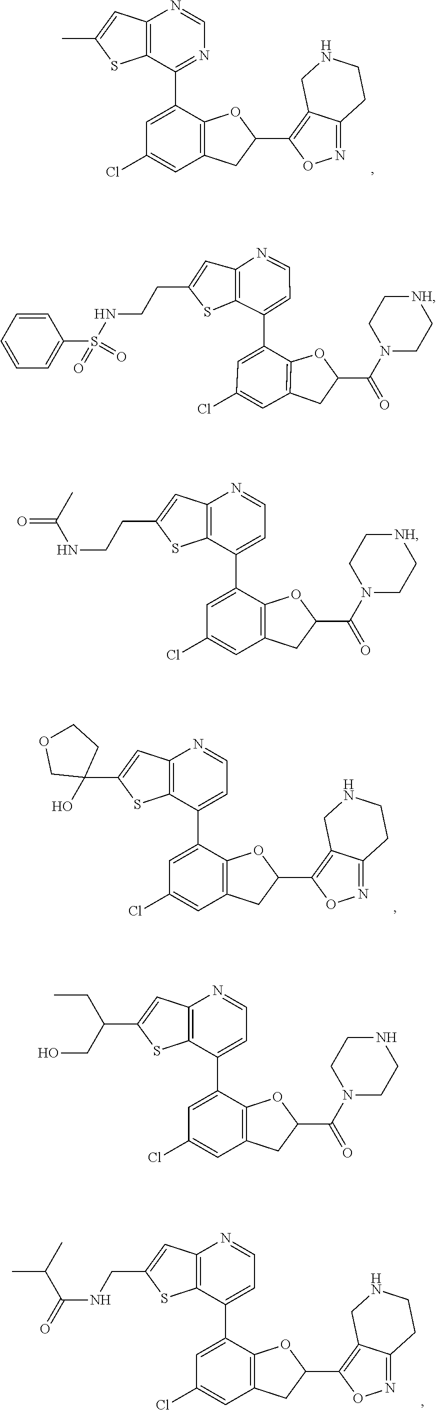 [SMILES: Cc1cc2ncnc(-c3cc(Cl)cc4c3OC(c3onc5c3CNCC5)C4)c2s1 ; O=C(C1Cc2cc(Cl)cc(-c3ccnc4cc(CCNS(=O)(=O)c5ccccc5)sc34)c2O1)N1CCNCC1 ; CC(=O)NCCc1cc2nccc(-c3cc(Cl)cc4c3OC(C(=O)N3CCNCC3)C4)c2s1 ; OC1(c2cc3nccc(-c4cc(Cl)cc5c4OC(c4onc6c4CNCC6)C5)c3s2)CCOC1 ; CCC(CO)c1cc2nccc(-c3cc(Cl)cc4c3OC(C(=O)N3CCNCC3)C4)c2s1 ; CC(C)C(=O)NCc1cc2nccc(-c3cc(Cl)cc4c3OC(c3onc5c3CNCC5)C4)c2s1]

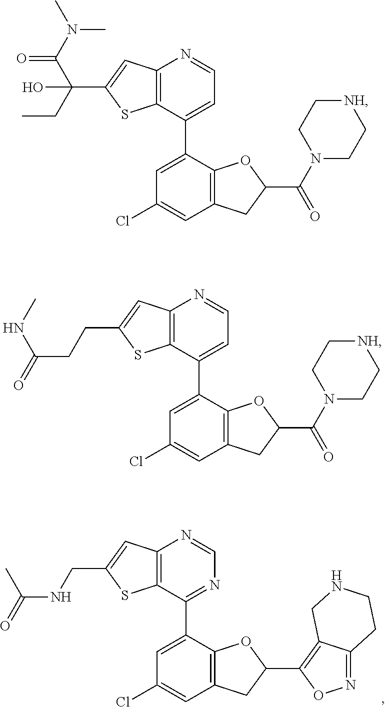 [SMILES: CCC(O)(C(=O)N(C)C)c1cc2nccc(-c3cc(Cl)cc4c3OC(C(=O)N3CCNCC3)C4)c2s1 ; CNC(=O)CCc1cc2nccc(-c3cc(Cl)cc4c3OC(C(=O)N3CCNCC3)C4)c2s1 ; CC(=O)NCc1cc2ncnc(-c3cc(Cl)cc4c3OC(c3onc5c3CNCC5)C4)c2s1]

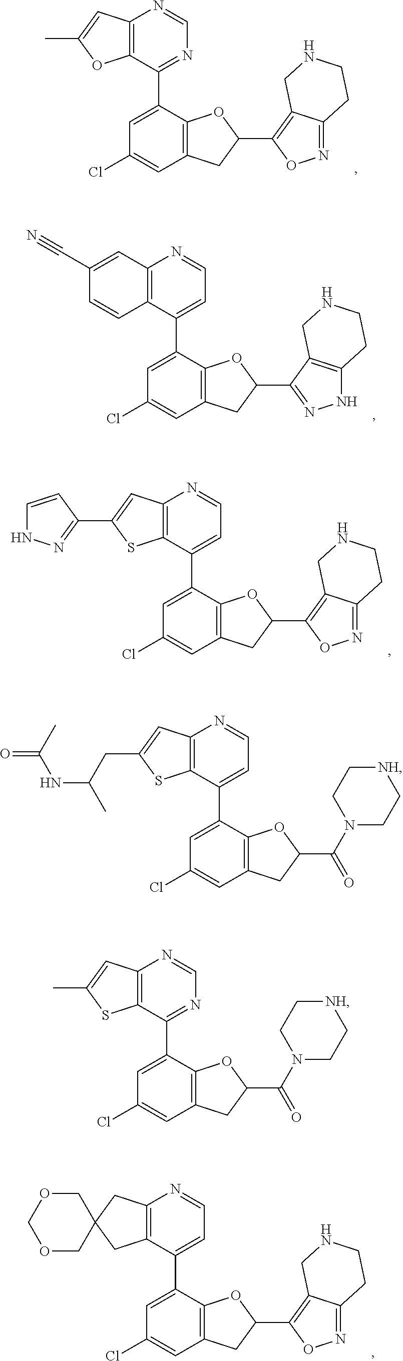 [SMILES: Cc1cc2ncnc(-c3cc(Cl)cc4c3OC(c3onc5c3CNCC5)C4)c2o1 ; N#Cc1ccc2c(-c3cc(Cl)cc4c3OC(c3n[nH]c5c3CNCC5)C4)ccnc2c1 ; Clc1cc2c(c(-c3ccnc4cc(-c5cc[nH]n5)sc34)c1)OC(c1onc3c1CNCC3)C2 ; CC(=O)NC(C)Cc1cc2nccc(-c3cc(Cl)cc4c3OC(C(=O)N3CCNCC3)C4)c2s1 ; Cc1cc2ncnc(-c3cc(Cl)cc4c3OC(C(=O)N3CCNCC3)C4)c2s1 ; Clc1cc2c(c(-c3ccnc4c3CC3(COCOC3)C4)c1)OC(c1onc3c1CNCC3)C2]

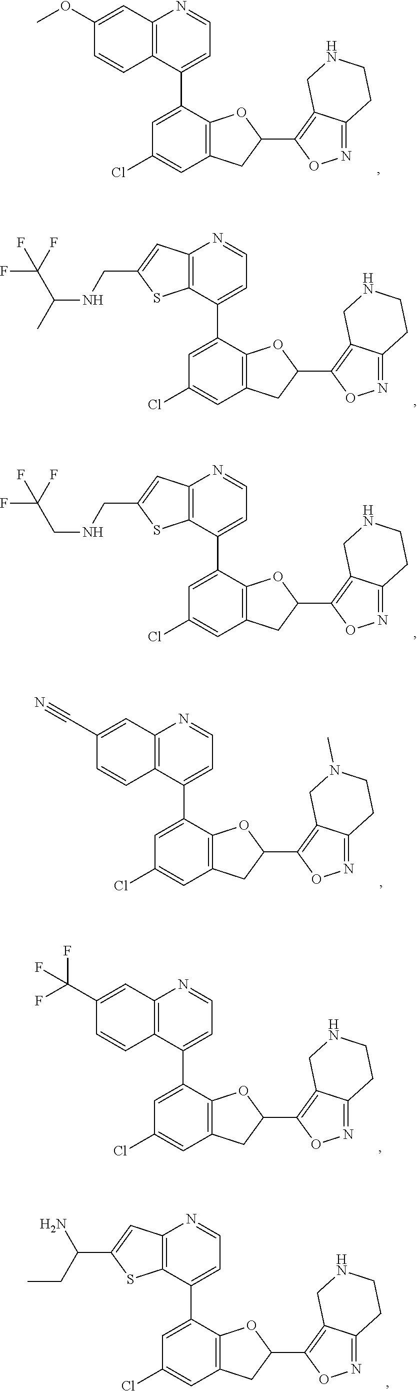 [SMILES: COc1ccc2c(-c3cc(Cl)cc4c3OC(c3onc5c3CNCC5)C4)ccnc2c1 ; CC(NCc1cc2nccc(-c3cc(Cl)cc4c3OC(c3onc5c3CNCC5)C4)c2s1)C(F)(F)F ; FC(F)(F)CNCc1cc2nccc(-c3cc(Cl)cc4c3OC(c3onc5c3CNCC5)C4)c2s1 ; CN1CCc2noc(C3Cc4cc(Cl)cc(-c5ccnc6cc(C#N)ccc56)c4O3)c2C1 ; FC(F)(F)c1ccc2c(-c3cc(Cl)cc4c3OC(c3onc5c3CNCC5)C4)ccnc2c1 ; CCC(N)c1cc2nccc(-c3cc(Cl)cc4c3OC(c3onc5c3CNCC5)C4)c2s1]

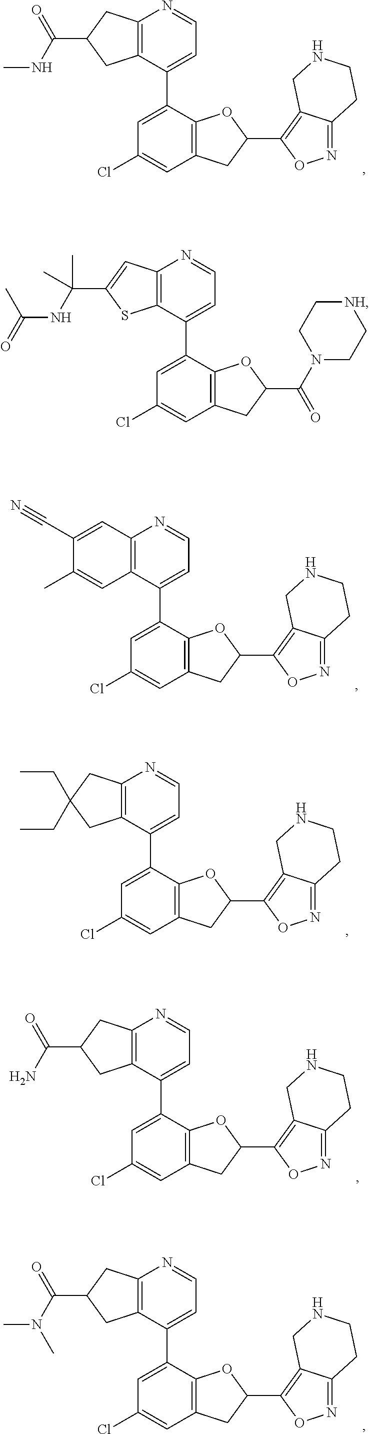 [SMILES: CNC(=O)C1Cc2nccc(-c3cc(Cl)cc4c3OC(c3onc5c3CNCC5)C4)c2C1 ; CC(=O)NC(C)(C)c1cc2nccc(-c3cc(Cl)cc4c3OC(C(=O)N3CCNCC3)C4)c2s1 ; Cc1cc2c(-c3cc(Cl)cc4c3OC(c3onc5c3CNCC5)C4)ccnc2cc1C#N ; CCC1(CC)Cc2nccc(-c3cc(Cl)cc4c3OC(c3onc5c3CNCC5)C4)c2C1 ; NC(=O)C1Cc2nccc(-c3cc(Cl)cc4c3OC(c3onc5c3CNCC5)C4)c2C1 ; CN(C)C(=O)C1Cc2nccc(-c3cc(Cl)cc4c3OC(c3onc5c3CNCC5)C4)c2C1]

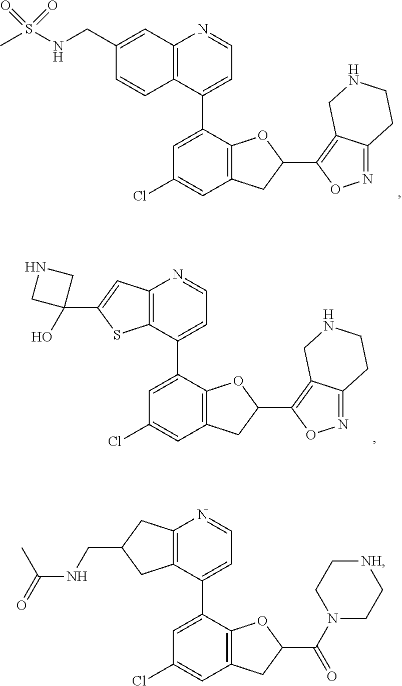 [SMILES: CS(=O)(=O)NCc1ccc2c(-c3cc(Cl)cc4c3OC(c3onc5c3CNCC5)C4)ccnc2c1 ; OC1(c2cc3nccc(-c4cc(Cl)cc5c4OC(c4onc6c4CNCC6)C5)c3s2)CNC1 ; CC(=O)NCC1Cc2nccc(-c3cc(Cl)cc4c3OC(C(=O)N3CCNCC3)C4)c2C1]

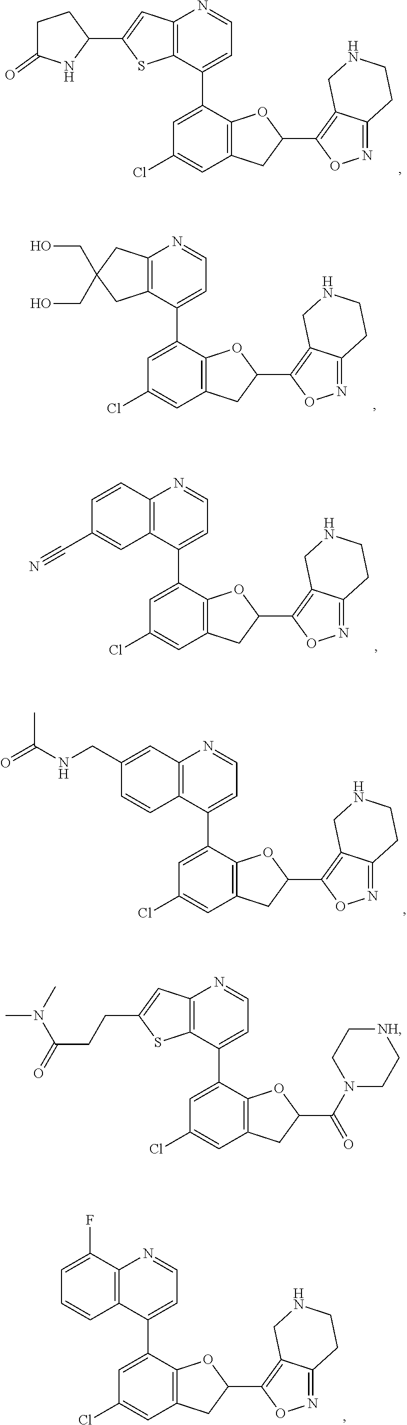 [SMILES: O=C1CCC(c2cc3nccc(-c4cc(Cl)cc5c4OC(c4onc6c4CNCC6)C5)c3s2)N1 ; OCC1(CO)Cc2nccc(-c3cc(Cl)cc4c3OC(c3onc5c3CNCC5)C4)c2C1 ; N#Cc1ccc2nccc(-c3cc(Cl)cc4c3OC(c3onc5c3CNCC5)C4)c2c1 ; CC(=O)NCc1ccc2c(-c3cc(Cl)cc4c3OC(c3onc5c3CNCC5)C4)ccnc2c1 ; CN(C)C(=O)CCc1cc2nccc(-c3cc(Cl)cc4c3OC(C(=O)N3CCNCC3)C4)c2s1 ; Fc1cccc2c(-c3cc(Cl)cc4c3OC(c3onc5c3CNCC5)C4)ccnc12]

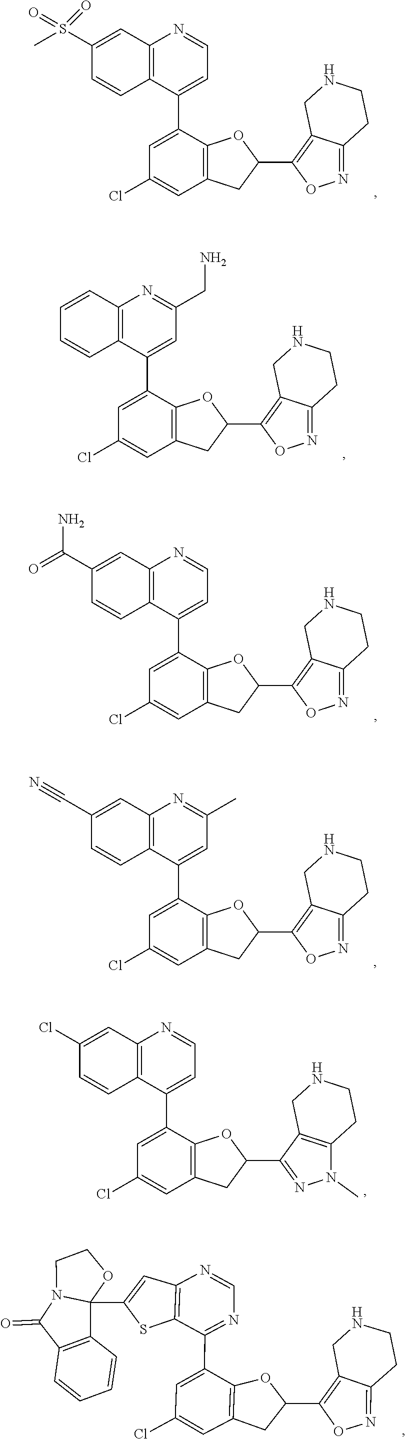 [SMILES: CS(=O)(=O)c1ccc2c(-c3cc(Cl)cc4c3OC(c3onc5c3CNCC5)C4)ccnc2c1 ; NCc1cc(-c2cc(Cl)cc3c2OC(c2onc4c2CNCC4)C3)c2ccccc2n1 ; NC(=O)c1ccc2c(-c3cc(Cl)cc4c3OC(c3onc5c3CNCC5)C4)ccnc2c1 ; Cc1cc(-c2cc(Cl)cc3c2OC(c2onc4c2CNCC4)C3)c2ccc(C#N)cc2n1 ; Cn1nc(C2Cc3cc(Cl)cc(-c4ccnc5cc(Cl)ccc45)c3O2)c2c1CCNC2 ; O=C1c2ccccc2C2(c3cc4ncnc(-c5cc(Cl)cc6c5OC(c5onc7c5CNCC7)C6)c4s3)OCCN12]

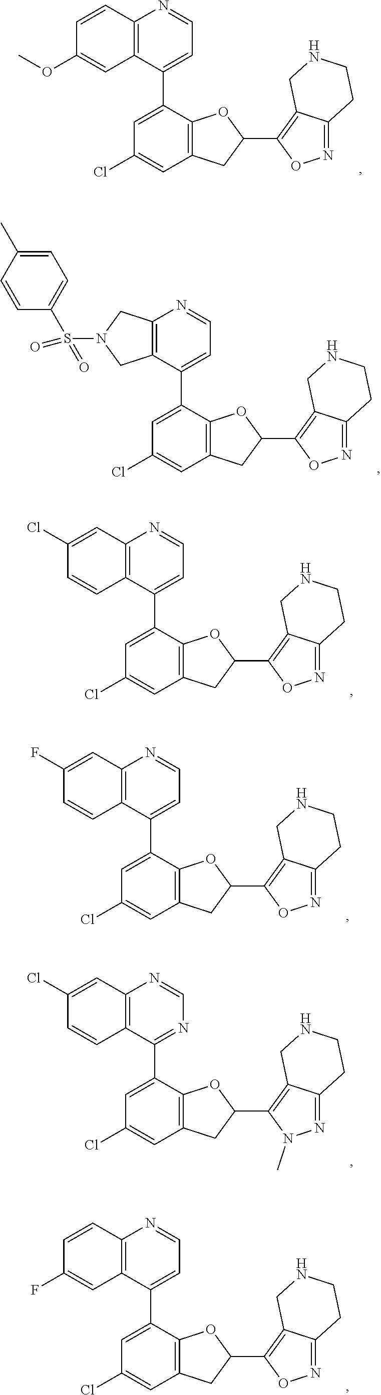 [SMILES: COc1ccc2nccc(-c3cc(Cl)cc4c3OC(c3onc5c3CNCC5)C4)c2c1 ; Cc1ccc(S(=O)(=O)N2Cc3nccc(-c4cc(Cl)cc5c4OC(c4onc6c4CNCC6)C5)c3C2)cc1 ; Clc1cc2c(c(-c3ccnc4cc(Cl)ccc34)c1)OC(c1onc3c1CNCC3)C2 ; Fc1ccc2c(-c3cc(Cl)cc4c3OC(c3onc5c3CNCC5)C4)ccnc2c1 ; Cn1nc2c(c1C1Cc3cc(Cl)cc(-c4ncnc5cc(Cl)ccc45)c3O1)CNCC2 ; Fc1ccc2nccc(-c3cc(Cl)cc4c3OC(c3onc5c3CNCC5)C4)c2c1]

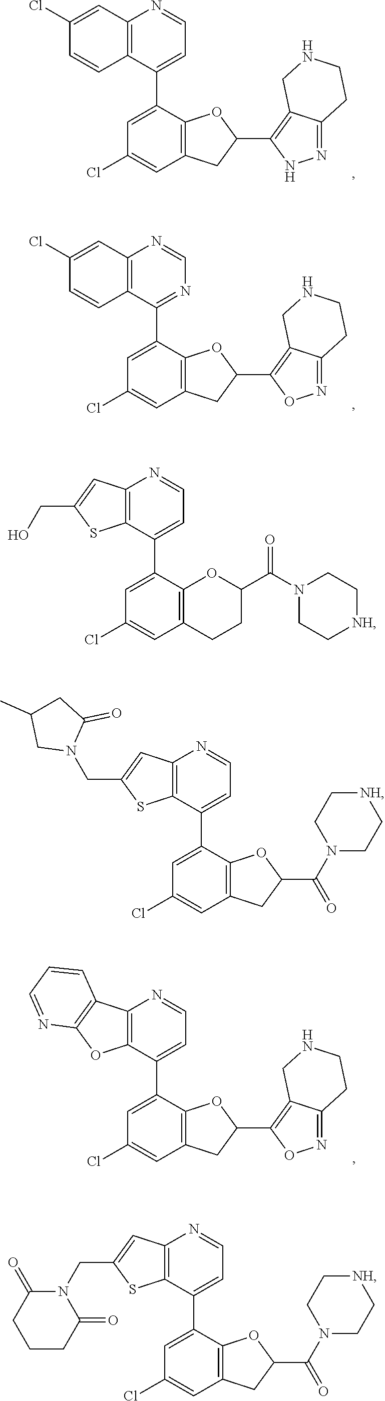 [SMILES: Clc1cc2c(c(-c3ccnc4cc(Cl)ccc34)c1)OC(c1[nH]nc3c1CNCC3)C2 ; Clc1cc2c(c(-c3ncnc4cc(Cl)ccc34)c1)OC(c1onc3c1CNCC3)C2 ; O=C(C1CCc2cc(Cl)cc(-c3ccnc4cc(CO)sc34)c2O1)N1CCNCC1 ; CC1CC(=O)N(Cc2cc3nccc(-c4cc(Cl)cc5c4OC(C(=O)N4CCNCC4)C5)c3s2)C1 ; Clc1cc2c(c(-c3ccnc4c3oc3ncccc34)c1)OC(c1onc3c1CNCC3)C2 ; O=C(C1Cc2cc(Cl)cc(-c3ccnc4cc(CN5C(=O)CCCC5=O)sc34)c2O1)N1CCNCC1]

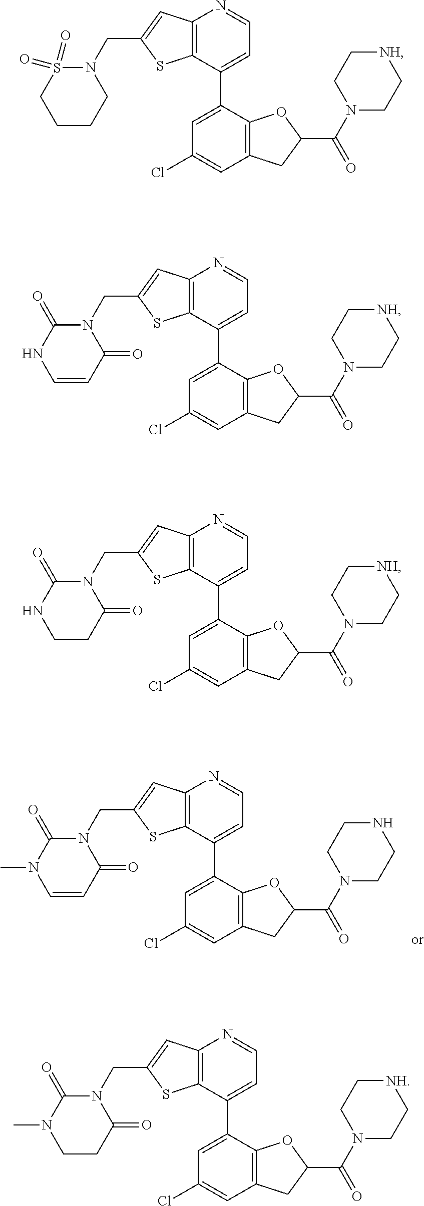 [SMILES: O=C(C1Cc2cc(Cl)cc(-c3ccnc4cc(CN5CCCCS5(=O)=O)sc34)c2O1)N1CCNCC1 ; O=C(C1Cc2cc(Cl)cc(-c3ccnc4cc(Cn5c(=O)cc[nH]c5=O)sc34)c2O1)N1CCNCC1 ; O=C(C1Cc2cc(Cl)cc(-c3ccnc4cc(CN5C(=O)CCNC5=O)sc34)c2O1)N1CCNCC1 ; Cn1ccc(=O)n(Cc2cc3nccc(-c4cc(Cl)cc5c4OC(C(=O)N4CCNCC4)C5)c3s2)c1=O ; CN1CCC(=O)N(Cc2cc3nccc(-c4cc(Cl)cc5c4OC(C(=O)N4CCNCC4)C5)c3s2)C1=O]

### Embodiment 62

A pharmaceutical composition, comprising the compound of any one of embodiments 1-61 and a pharmaceutically acceptable excipient.

### Embodiment 63

A method of inhibiting ubiquitin-specific-processing protease 7 (USP7), comprising contacting USP7 with a compound of any one of embodiments 1-61 or the pharmaceutical composition of embodiment 62.

### Embodiment 64

A method of treating a USP7-mediated disease or disorder, comprising administering to a patient in need thereof a therapeutically effective amount of a compound of any one of embodiments 1-61 or the pharmaceutical composition of embodiment 62.

### Embodiment 65

The method of embodiment 64, wherein the USP7-mediated disease or disorder is an immune disorder, a cardiovascular disease, a viral infection, inflammation, a metabolism/endocrine disorder or a neurological disorder.

### Embodiment 66

The method of embodiment 64, wherein the USP7-mediated disease or disorder is cancer.

### Embodiment 67

The method of embodiment 66, wherein the cancer is breast cancer, ovarian cancer, cervical cancer, prostate cancer, cancer of the testes, genitourinary tract cancer, cancer of the esophagus, cancer of the larynx, glioblastoma, neuroblastoma, stomach cancer, skin cancer, keratoacanthoma, lung cancer, epidermoid carcinoma, large cell carcinoma, non-small cell lung carcinoma (NSCLC), small cell carcinoma, lung adenocarcinoma, bone cancer, colon cancer, adenoma, pancreatic cancer, adenocarcinoma, thyroid cancer, follicular carcinoma, undifferentiated carcinoma, papillary carcinoma, seminoma, melanoma, sarcoma, bladder carcinoma, liver carcinoma and biliary passages, kidney carcinoma, lymphoma, cancer of the pharynx, cancer of the lip, cancer of the tongue, cancer of the mouth, cancer of the small intestine, colorectal cancer, cancer of the large intestine, rectal cancer, brain cancer Hodgkin's, leukemia, cancer of the bronchus, cancer of the thyroid, liver cancer, intrahepatic bile duct cancer, gastric cancer, glioma/glioblastoma, endometrial cancer, melanoma, kidney cancer, renal cancer, pelvic cancer, urinary bladder cancer, multiple myeloma, acute myelogenous leukemia, chronic myelogenous leukemia, lymphocytic leukemia, chronic lymphoid leukemia (CLL), myeloid leukemia, cancer of the oral cavity and pharynx, non-Hodgkin lymphoma, melanoma or villous colon adenoma.

### Embodiment 68

The method of embodiment 66, further comprising administering to the patient a chemotherapeutic agent in combination with the compound of any one of embodiments 1-61 or the pharmaceutical composition of embodiment 62.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

Identification of USP7 Inhibitors:

In embodiments, compounds described herein possess at least one property or characteristic that is of therapeutic relevance. Candidate inhibitors may be identified by using, for example, an art-accepted assay or model. The Example section described assay(s) that were used to determine the usp7 inhibitory activity of the compounds described herein, as well as assays that could be used to evaluate one or more characteristics of the compounds; the skilled artisan is aware of other procedures, assay formats, and the like that can be employed to generate data and information useful to assess the USP7 inhibitors described herein.

After identification, candidate inhibitors can be further evaluated by using techniques that provide data regarding characteristics of the inhibitors (e.g., pharmacokinetic parameters). Comparisons of the candidate inhibitors to a reference standard (which may the “best-of-class” of current inhibitors) are indicative of the potential viability of such candidates. USP7 inhibitors that can serve as reference or benchmark compounds include those shown to demonstrate desired activity and characteristics useful for analyzing candidate inhibitors which will be apparent to the skilled artisan.

Chemical Syntheses

Definition of abbreviations used: DCM=dichloromethane; THF=tetrahydrofuran; MTBE=Methyl tert-butyl ether; EtOAc=Ethylacetate; DMF=dimethylformamide; DMSO=dimethylsulfoxide; DIEA=diisopropylethylamine; DEA=diethylamine; NaOH=sodium hydroxide; KOH=potassium hydroxide; DMAP=4-Dimethylaminopyridine; mCPBA=meta-Chloroperoxybenzoic acid; HATU=1-[Bis(dimethylamino)methylene]-1H-1,2,3-triazolo[4,5-b]pyridinium 3-oxid hexafluorophosphate; TBAF=Tetra-n-butylammonium fluoride; TEMPO=2,2,6,6-Tetramethylpiperidin-1-yl)oxyl; T3P=Propane Phosphonic Acid Anhydride; TBSCl=tert-Butyldimethylsilyl chloride; AcOK=potassium acetate; DBU=1,8-Diazabicyclo[5.4.0]undec-7-ene; HCl=hydrochloric acid; TFA=trifluoroacetic acid; TLC=Thin Layer Chromatography; HPLC=high pressure liquid chromatography; rt=room temperature.

### General Synthetic Schemes

### General procedure to prepare 7-bromo-2,3-dihydrobenzofuran-2-carboxylic Acid

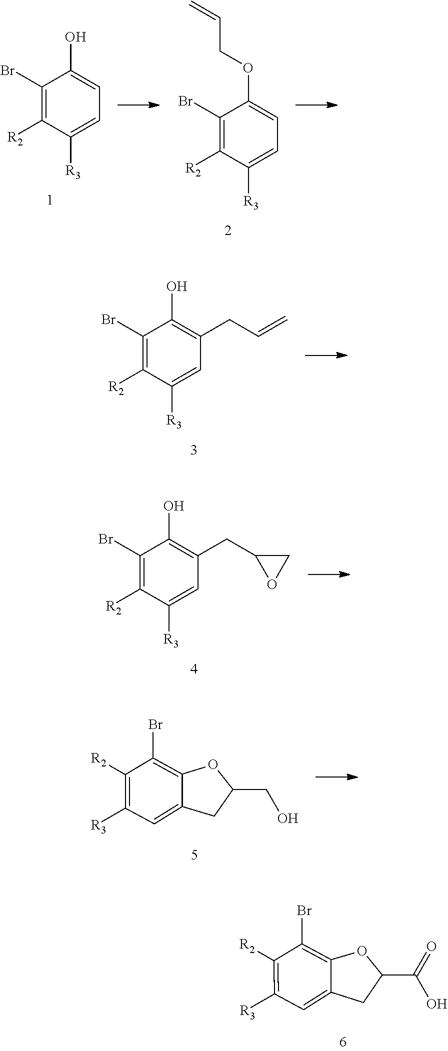

To bromo-phenol of general structure 1 an organic solvent such as DMF is added alkaline carbonate such as potassium carbonate and 3-bromoprop-1-ene. Let stir under heating preferably around 60 OC between 2-4 h. The reaction is diluted with an aqueous basic solution such as 1M NaOH and an organic solution such as MTBE and extracted with an organic solvent such as MTBE, dried over a drying agent such as sodium sulfate, filtered through a silica plug and concentrated to afford the title compound 2. The title compound 2 was used directly in next step as crude.

To a solution of general structure 2 is added mesitylene. The reaction is purged with an inert gas such as N₂ and heated at 170° C. (4-16 h). The reaction is allowed to cool to room temperature. Solvent was removed under reduced pressure to afford the title compound 3. The title compound 3 was used directly in next step as crude.

A solution of general structure 3 in an organic solvent such as DCM is cooled down to a temperature of 0 OC using an ice bath. A source of strong oxidizing agent such as mCPBA is added portionwise. The reaction is allowed to warm to room temperature and stirred (8-18 h) under room temperature. The reaction is quenched with 2M Na₂S2O₃, diluted with water, extracted with an organic solvent such as DCM. The organic layers are combined, dried over a drying agent such as magnesium sulfate, filtered. Solvent is removed under reduced pressure to afford the title compound 4 which was used as crude for the next step.

To a solution of general structure 4 in polar aprotic solvent such as DMSO and water at 0° C. is added strong base such as KOH. The reaction mixture is stirred at 0° C. (1-2) h and is warmed to room temperature. The reaction mixture is continued stirring at room temperature (8-16 h). The reaction mixture is acidified with a strong acid such as HCl to pH around 5. It is extracted with an organic solvent such as MTBE. Combined organics are dried over a drying agent such as sodium sulfate, filtered through a silica pad and concentrated under reduced pressure. The residue can be purified by silica gel chromatography using a mixture of organic solvents for example a mixture of EtOAc and Hexanes to give compounds of the general formula 5.

A solution of general structure 5 in aprotic solvent such as THF was cooled to 0° C. is added an oxidizing agent such as TEMPO, an ionic salt such as KBr, a weak base such as saturated sodium bicarbonate, and a chloride source such as bleach. The reaction mixture is gradually warmed to room temperature and stirred at room temperature (1-2 h). The reaction mixture is acidified with a strong acid such as HCl to pH around 2. It is extracted with an organic solvent such as EtOAc. Combined organics were dried over a drying agent such as sodium sulfate, filtered and concentrated under reduced pressure. Solid was trituated with a combination of organic solvents such as Et2O/hexane (10:1) to afford the title compound of general formula 6.

### General Procedure to Prepare (R)-7-bromo-2,3-dihydrobenzofuran-2-carboxylic Acid

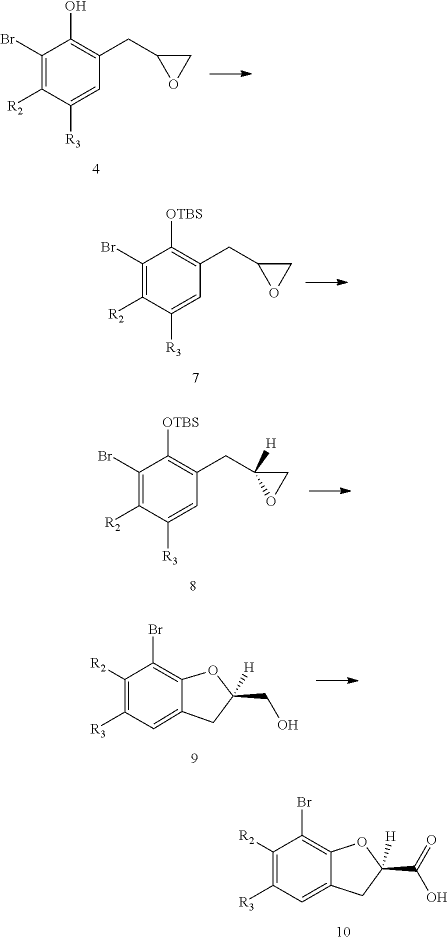

To a solution of general structure 4 in an organic solvent such as DCM at 0° C. is added a silylation agent such as TBSCl, an organic base such as triethylamine followed by a catalytic amount of strong base such as DMAP. The reaction is stirred at temperature (2-4 h). Reaction mixture is washed with water, dried over a drying agent such as sodium sulfate, concentrated in vacuo. The crude residue is purified by silica gel chromatography using a mixture of organic solvents for example a mixture of EtOAc and Hexanes to give compounds of the general formula 7.

To a Jacobsen's catalyst such as (S,S)-Co(II)-salen is added a mild acid such as acetic. The resulting mixture is stirred open to air for 30 min, then concentrated in vacuo. To this residue is added the compound of general formula 7 followed by the addition of water. An aprotic solvent such as THF is added until water all dissolve then capped with a glass stopper and let stir overnight. The solvent is removed under reduced pressure. The crude residue is purified by silica gel chromatography using a mixture of organic solvents for example a mixture of EtOAc and Hexanes to give compounds of the general formula 8.

To the compound of general formula 8 in aprotic solvent such as THF at 0° C. was added a source of fluoride ions in organic solvent such as TBAF. The reaction mixture was stirred for 1 h and then at room temperature at which a metal carbonate such as calcium carbonate, ion exchange resin such as Amberlyst 16, and a polar protic solvent such as methanol are added. The reaction mixture is continued stirring at room temperature for 1 h. The mixture is filtered over celite, washed with methanol, and concentrated in vacuo to give compounds of the general formula 9.

A solution of general structure 9 in aprotic solvent such as THF was cooled to 0° C. is added an oxidizing agent such as TEMPO, an ionic salt such as KBr, a weak base such as saturated sodium bicarbonate, and a chloride source such as bleach. The reaction mixture is gradually warmed to room temperature and stirred at room temperature (1-2 h). The reaction mixture is acidified with a strong acid such as HCl to pH around 2. It is extracted with an organic solvent such as EtOAc. Combined organics were dried over a drying agent such as sodium sulfate, filtered and concentrated under reduced pressure. Solid is trituated with a combination of organic solvents such as Et2O/hexane (10:1) to afford the title compound of general formula 10.

General synthesis of 7-bromo-5-chloro-2,3-dihydrobenzofuran-2-carboxamide, wherein R⁷ is as described herein, and R⁵ and R⁶ correspond to a substituent described herein (e.g., R⁹ or R¹⁰, and embodiments thereof)

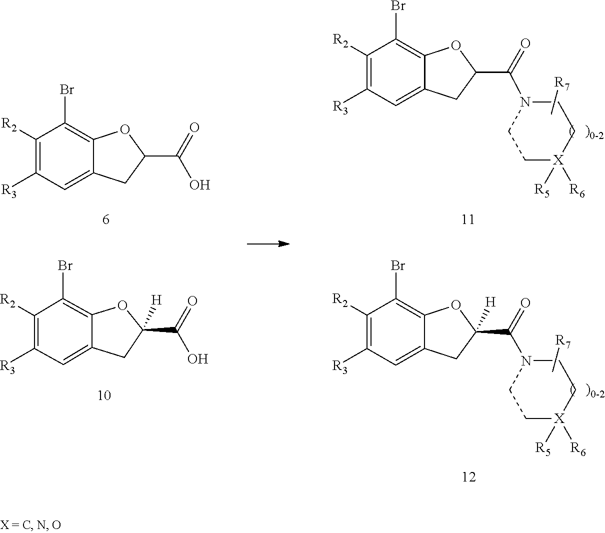

To a solution the title compound of general formula 6 or 10, amines and an organic base such as methylimidazole in a combination of polar solvent such as EtOAc/DMF (1:2) is added a coupling reagent such as T3P. The resulting reaction is stirred at room temperature (2-16 h). The reaction mixture is diluted with basic aqueous solution such as NaOH solution, extracted with an organic solvent such as EtOAc, dried over a drying agent such as sodium sulfate, filtered and concentrated in vacuo. The crude residue is purified by silica gel chromatography using a mixture of organic solvents for example a mixture of EtOAc and Hexanes to give compounds of the general formula 11 or 12.

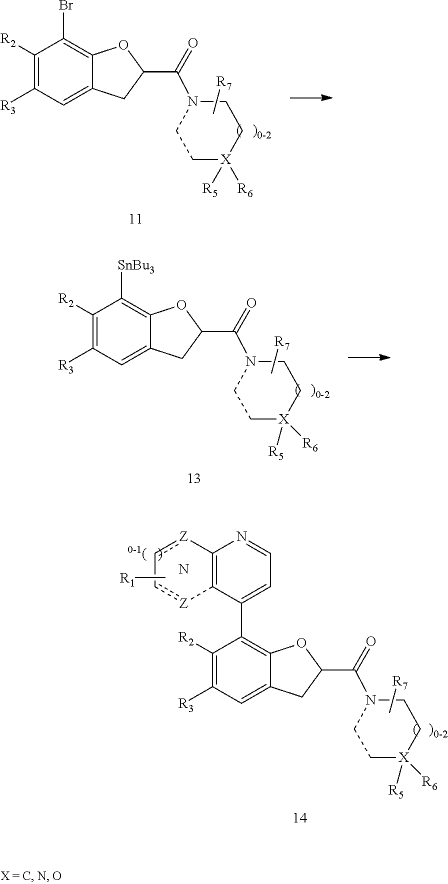

A solution of compounds of the general formula 11 and catalytic Pd source such as PdCl₂(PPh₃)₂ in a polar solvent such as dioxane is evacuated/backfilled with argon. To this mixture is added tributyl(tributylstannyl)stannane. The resulting mixture is stirred at 110° C. for (6-18 h). Reaction mixture is allowed to cool to room temperature and solvent is removed under reduced pressure. The crude residue is purified by silica gel chromatography using a mixture of organic solvents for example a mixture of EtOAc and Hexanes to give compounds of the general formula 13.

A solution of compounds of the general formula 13, a Pd source such as PdCl₂(PPh₃)₂, halide pyridine and copper oxide in an organic solvent such as dioxane is spared with argon for 5 min and heated to 100° C. with stirring (4-16 h). The solvent is removed under reduced pressure. The crude residue is purified by silica gel chromatography using a mixture of organic solvents for example a mixture of EtOAc and hexanes to give compounds of the general formula 14.

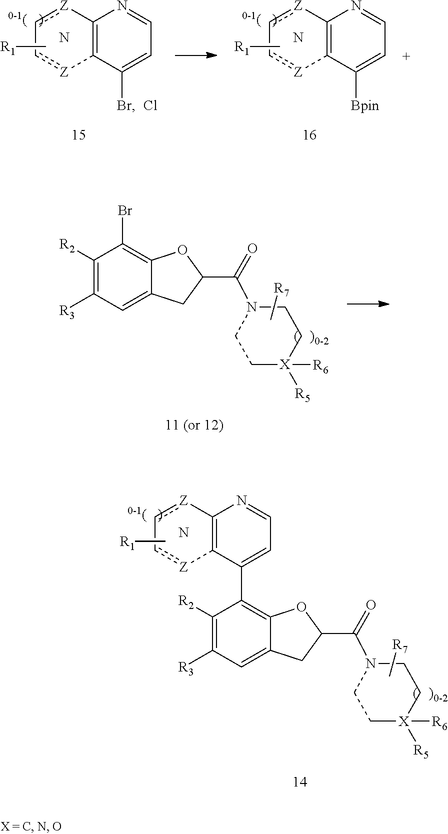

In a certain instance compounds of general structure 14 can be made via Suzuki coupling. A solution of halide pyridine of general structure 15, Bis(pinacolato)diboron, a Pd source such as PdCl₂dppf, AcOK in an organic solvent such as dioxane is heated to 100° C. with stirring (2-4 h). The reaction mixture is allowed to warm to room temperature and diluted with water, extracted with an organic solvent such as ethyl acetate, dried over a drying agent such as sodium sulfate, filtered, and concentrated under reduced pressure to afford the title compound of general structure 16 which was used directly for the next step.

To compounds of general structure 11 (or 12), compounds of general structure 16, a Pd source such as PdCl₂(PPh₃)₂ in a combination of organic solvents such as toluene/ethanol (1:1), and an aqueous basic solution such as aq. sodium carbonate is stirred vigorously at 100° C. (2-16 h). The reaction mixture is warmed to room temperature, diluted with H₂O. The mixture is extracted with an organic solvent such as EtOAc. Organic layers are combined, dried over a drying agent such as sodium sulfate and concentrated in vacuo. The crude residue is purified by silica gel chromatography using a mixture of organic solvents for example a mixture of EtOAc and hexanes to give compounds of the general formula 14.

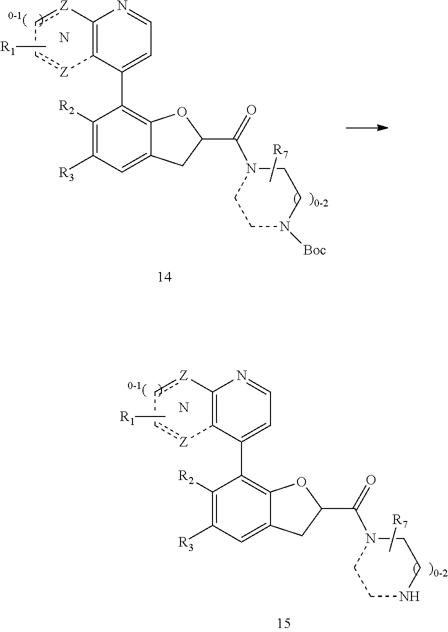

When compounds of the general formula 14 having X=N, R⁵=protecting group, the protective group on compounds of general structure 14 can be exposed to an acidic organic solution, for example HCl in dioxane or trifluoroacetic acid in DCM. The mixture is stirred at room temperature for a time between one and 16 hours. The reaction mixture can be concentrated under reduced pressure to give an amine salt of the general structure 15.

### General Synthesis of 7-bromo-5-chloro-2,3-dihydrobenzofuran-2-isoxazole

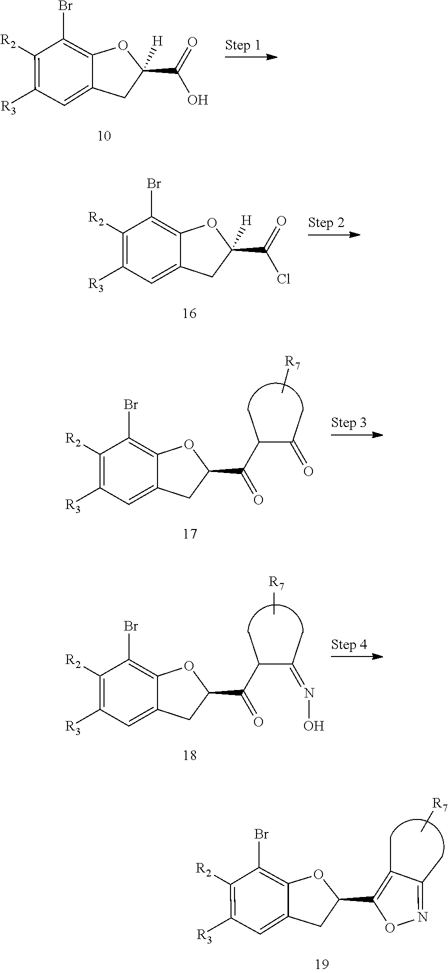

To the compound of general formula 10 in aprotic solvent such as THF at cool temperature such as 0 OC is added a source of acid chlorides such as oxalyl chloride. The resulting mixture is stirred at rt for 1 h and the mixture is reduced under vacuum to give the desired title compound 16 which is used in the next step without further purification.

To a ketone such as tert-butyl 4-oxopiperidine-1-carboxylate or 1,4-dioxaspiro[4.5]decan-8-one in aprotic solvent such as THF at −78° C. is added a strong cryogenic base such as LHMDS. The resulting mixture is continued stirring at −78° C. for about 1 h at which 16 in a small amount of aprotic solvent such as THF is added. The reaction mixture is continued stirring at −78° C. for about 1 h and then warmed to 0° C. The mixture is then quenched with a mildly acidic aqueous solution such as NH₄Cl and extracted with an organic solvent such as EtOAc. The combined organic layers are combined and concentrated. The crude residue is purified by silica gel chromatography using a mixture of organic solvents for example a mixture of EtOAc and hexanes to give compounds of the general formula 17.

To the compound of general formula 17 in a polar organic solvent such as EtOH is added NH₂OH—HCl salt and a base such as NaOAc. The reaction mixture is heated to 80° C. for about 2 h.

The reaction mixture is cooled and concentrated. The reaction mixture is diluted with water and extracted with an organic solvent such as EtOAc. The organic layers are combined and washed with mild basic aqueous solution such as NaHCO₃, concentrated to afford the title compound 18 which was used as crude for the next step.

To the compound of general formula 19 in an organic solvent such as DCM is added MsCl following by the addition of an organic base such as TEA. The reaction is stirred at rt for about 2 h and concentrated. The reaction mixture was extracted with an organic solvent such as EtOAc, washed with a acidic aqueous solvent such as 1M HCl, water, and brine. The organic layers are concentrated. The crude residue is purified by silica gel chromatography using a mixture of organic solvents for example a mixture of EtOAc and hexanes to give compounds of the general formula 19.

### General Synthesis of the 7-bromo-2,3-dihydrobenzofuran-2-pyrazole

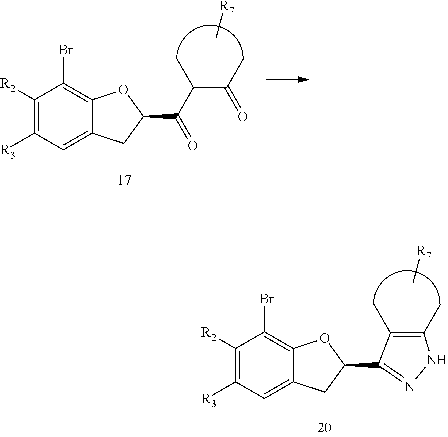

To the compound of general formula 17 in a polar organic solvent such as EtOH is added hydrazine and a base such as NaOAc. The reaction mixture is heated to 80° C. for about 2 h. The reaction mixture is cooled and concentrated. The reaction mixture is diluted with water and extracted with an organic solvent such as EtOAc. The organic layers are combined and washed with mild basic aqueous solution such as NaHCO₃, concentrated to afford the title compound 20.

General synthesis of the 7-bromo-2,3-dihydrobenzofuran-2-oxazole, wherein R⁵⁶ shown below are each independently an substituent described herein (e.g., R³⁹).

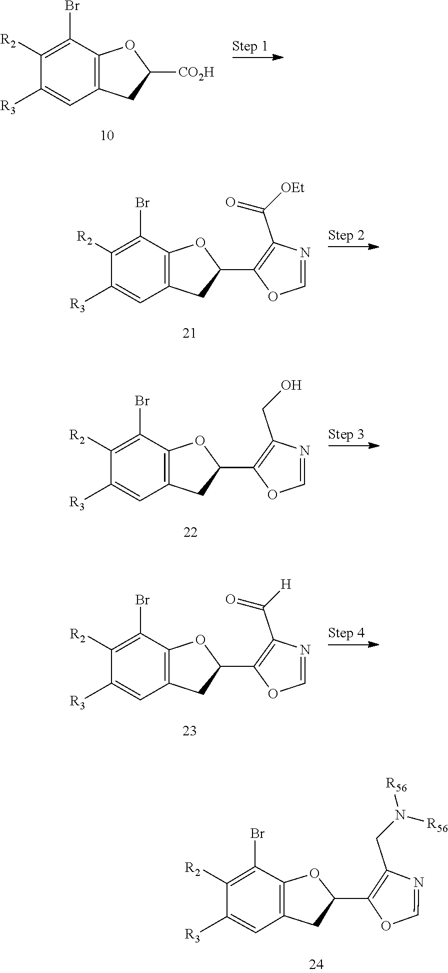

To the compound of general formula 10 in a polar organic solvent such as DMF at 0° C. to rt is added diphenylphosphoryl azide, and a base such as sodium carbonate. The reaction is stirred at rt for 12-24 hr, diluted with water, and extracted with EtOAc. The organic layers are washed with water and brine, and dried over a dessicant such as magnesium sulfate or sodium sulfate. Purification by chromatography gives the title compound.

To the compound of general formula 21 in a mixture of an organic solvent such as THF and water, is added a reducing agent such as sodium borohydride. The mixture is stirred for 24-72 hr, diluted with water then extracted with an organic solvent such as EtOAc. The reaction mixture was diluted with water (50 mL) then extracted with EtOAc. The organic phases are dried over a dessicant such as magnesium sulfate or sodium sulfate. Purification by chromatography gives the title compound.

To the compound of general formula 22 in an organic solvent such as DCM is added a base such as sodium carbonate an oxidant such as pyridinium chlorochromate or Dess-Martin Periodinane. The reaction mixture is filtered to remove the solids then concentrated. Purification by chromatography gives the title compound.

To the compound of general formula 23 in an organic solvent such as DCM or DCE is added an organic amine such as dimethyl amine followed by a reducing agent such as sodium triacetoxyborohydride. The reaction is quenched with an aqueous base such as sodium hydroxide and the aqueous phase extracted with DCM. The organic phases are dried over a dessicant such as magnesium sulfate or sodium sulfate. Purification by chromatography gives the title compound.

### General Synthesis of the 8-bromochromane-2-carboxylic Acid

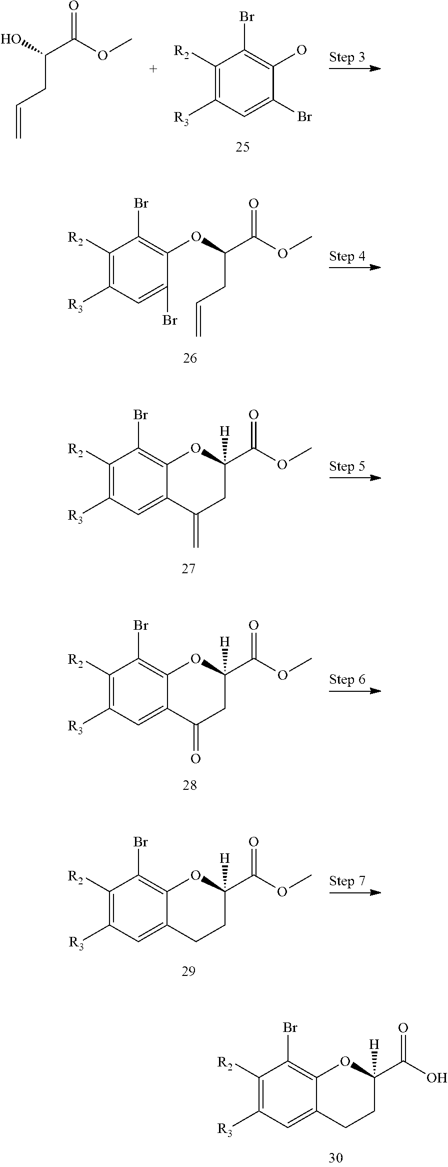

To a solution of a compound of general formula 25 and methyl (S)-2-hydroxypent-4-enoate in a polar solvent such as THF is added diisopropyl azodicarboxylate and triphenylphosphine and the mixture is stirred overnight (rt or 50° C.). The solvent is removed under reduced pressure and the crude residue is purified by silica gel chromatography using a mixture of organic solvents for example a mixture of EtOAc and hexanes to give compounds of the general formula 26.

To a solution of a compound of general formula 26 in a polar solvent such as DMF or DME is added a palladium source such as Pd(OAc)₂, a ligand such as 1,2-bis(diphenylphosphino)ethane, and a base such as potassium acetate and the mixture is heated at 90-150° C. for 16-36 hours. The reaction mixture is cooled to rt and diluted with water. The mixture is extracted with an organic solvent such as EtOAc. Organic layers are combined, dried over a drying agent such as sodium sulfate and concentrated in vacuo. The crude residue is purified by silica gel chromatography using a mixture of organic solvents for example a mixture of EtOAc and EtOAc to give compounds of the general formula 27.

Into a solution of a compound of general formula 27 in a solvent such as DCM at −78° C. is bubbled O₃ (g) for 10-30 min. A reducing agent such as dimethyl sulfide or PPh₃ is added and the resulting mixture is slowly warmed to rt and concentrated in vacuo. The crude residue is purified by silica gel chromatography using a mixture of organic solvents for example a mixture of EtOAc and EtOAc to give compounds of the general formula 28.

To a solution of a compound of general formula 28 is added a reducing agent such as triethylsilane and TFA and the mixture is heated at 50-90° C. for 8-24 h in a sealed tube. The crude residue is purified by silica gel chromatography using a mixture of organic solvents for example a mixture of EtOAc and EtOAc to give compounds of the general formula 29.

To a solution of a compound of general formula 29 in a polar solvent such as MeOH or EtOH is added an aqueous solution of an inorganic base such as sodium hydroxide at 0° C.-rt. The mixture is stirred for 1-24 hours, concentrated, treated with an aqueous acid such as HCl, and extracted with an organic solvent such as EtOAc. The combined organic phases are dried over a dessicant such as magnesium sulfate or sodium sulfate, filtered, and concentrated to give compounds of type 30.

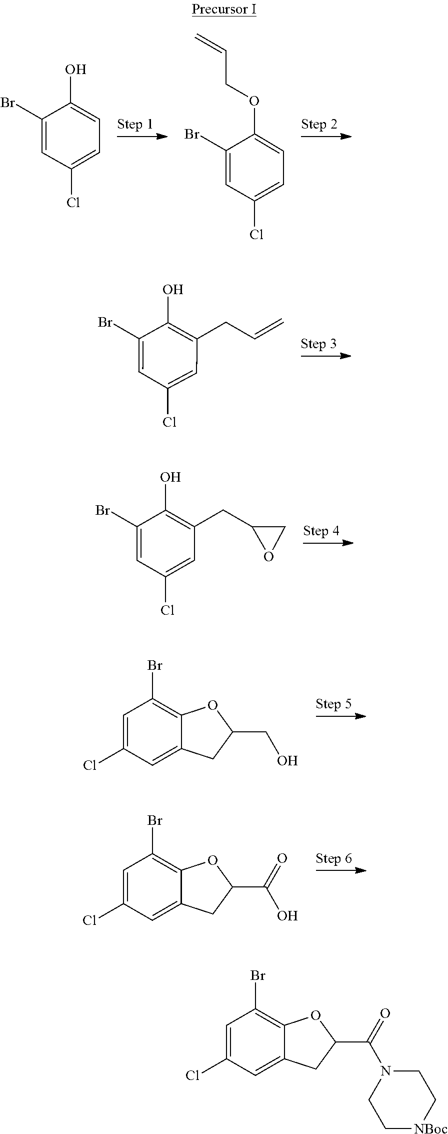

### Step 1: 1-(allyloxy)-2-bromo-4-chlorobenzene

To 2-bromo-4-chloro-phenol (65.5 g, 316 mmol) in DMF (100 mL) was added Potassium Carbonate (52.3 g, 379 mmol) and 3-bromoprop-1-ene (42 g, 347 mmol). The resulting mixture was let stir at 60° C. (water bath) for 3 h. The reaction diluted with 1M NaOH (200 mL) and MTBE (250 mL) and extracted 3× with 100 mL MTBE, dried with Na₂SO₄, filtered through a silica plug and concentrated to afford the title compound. The title compound was used directly in next step as crude (12 g, 15% yield). [M+H] 247.

### Step 2: 2-allyl-6-bromo-4-chlorophenol

To 2-allyl-6-bromo-4-chloro-phenol (58.6 g, 339 mmol) was added mesitylene (170 mL). The reaction was purged with N₂ and heated at 170° C. with condenser attached (no running water) under argon (4-16 h). The reaction was allowed to cool to room temperature. Solvent was removed under reduced pressure to afford the title compound (73.3 g, 87% yield). [M+H] 247.

### Step 3: 2-bromo-4-chloro-6-(oxiran-2-ylmethyl)phenol

A solution of 2-allyl-6-bromo-4-chloro-phenol (29.3 g, 118 mmol) in DCM (500 mL) was cooled down to 0° C. using an ice bath. mCPBA (60 g, 242 mmol) was added portionwise. The reaction was allowed to warm to RT. The reaction was stirred overnight under room temperature. The reaction was quenched with 2M Na₂S2O₃ solution (aq., 100 mL), diluted with water (100 mL), quenched with saturated aq. sodium bicarbonate (200 mL). It was extracted DCM (2×200 mL), dried and filtered. The solvent was removed under reduced pressure to afford the title compound (60.5 g, 95% yield). [M+H] 263.

### Step 4: (7-bromo-5-chloro-2,3-dihydrobenzofuran-2-yl)methanol

To 2-bromo-4-chloro-6-(oxiran-2-ylmethyl)phenol (60.5 g, 230 mmol) in DMSO (230 mL) and water (200 mL) at 0° C. was added KOH (16.4 g, 298 mmol). The reaction mixture was stirred for 1 h and monitored by TLC. The reaction mixture was warmed to room temperature. The reaction mixture was continued stirring at room temperature for overnight. The reaction mixture was acidified with 1M HCl to pH around 5. It was extracted with MTBE (100 mL, 4×). Combined organics were dried, filtered through a silica pad and concentrated under reduced pressure. The crude residue was purified by flash column chromatography (silica gel, 0-30% EtOAc in hexanes) to afford the title compound (51 g, 84% yield). [M+H] 263.

### Step 5: 7-bromo-5-chloro-2,3-dihydrobenzofuran-2-carboxylic acid

A solution of (7-bromo-5-chloro-2,3-dihydrobenzofuran-2-yl)methanol (10 g, 37.9 mmol) in THF (76 mL) was cooled to 0° C. was added TEMPO (1.19 g, 7.59 mmol), KBr (1.13 g, 9.49 mmol), saturated sodium bicarbonate (10 mL) and bleach (15 mL, 85.8 mmol). The reaction mixture was gradually warmed to room temperature and stirred at room temperature for 2 h. The reaction mixture was diluted with 1M HCl to pH around 2. It was extracted with EtOAc (25 mL, 4×). Combined organics were dried, filtered and concentrated under reduced pressure. Solid was trituated with Et₂O/hexanes (10:1) to afford the title compound as white solid (10 g, 95% yield). [M+H] 277.

### Step 6: tert-butyl 4-(7-bromo-5-chloro-2,3-dihydrobenzofuran-2-carbonyl)piperazine

1-carboxylate. To a solution of 7-bromo-5-chloro-2,3-dihydrobenzofuran-2-carboxylic acid (4.16 g, 15 mmol), N-Boc-piperazine (5.59 g, 30 mmol) and methylimidazole (3.69 g, 45 mmol) in EtOAc (30 mL) was added T3P (9.55 g, 30 mmol). The resulting mixture was stirred at room temperature for 30 min. The reaction mixture was diluted with NaOH aq. (1M, 2 mL), extracted with EtOAc, dried, filtered and concentrated under reduced pressure. The crude residue was purified by flash column chromatography (silica gel, 20-100% EtOAc in hexanes) to afford the title compound as white foam (6.4 g, 96% yield). [M+H] 445.

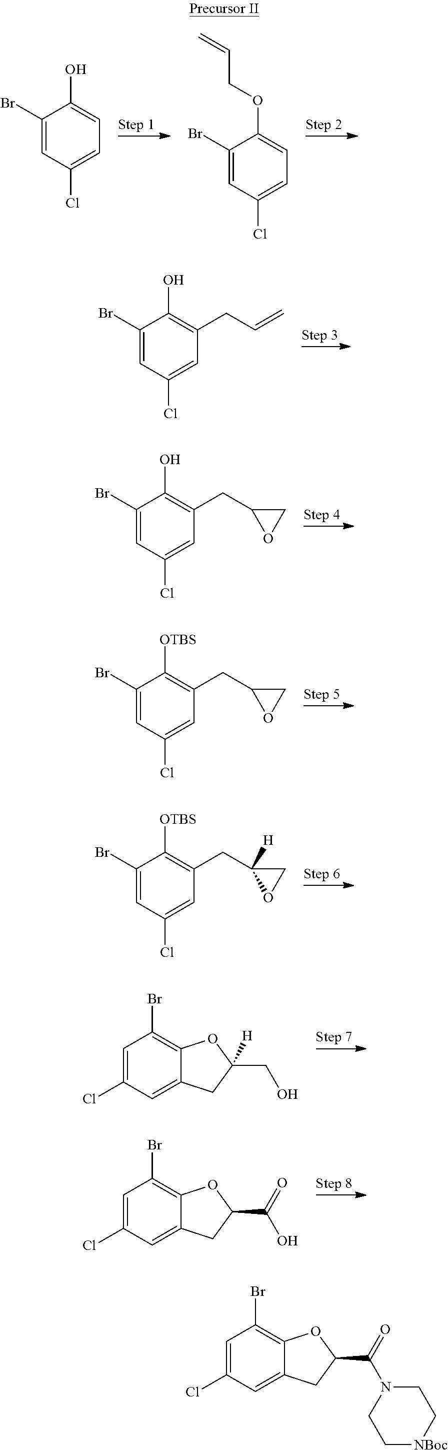

### Step 1: 1-(allyloxy)-2-bromo-4-chlorobenzene

To 2-bromo-4-chloro-phenol (58.5 g, 282 mmol) in DMF (165.88 mL) was added Potassium Carbonate (46.7 g, 338.39 mmol) and 3-bromoprop-1-ene (5.64 mL, 65.22 mmol). The resulting mixture was let stir at 60° C. for 3 h. The reaction diluted with 1M NaOH (200 mL) and MTBE (250 mL) and extracted 3× with 100 mL MTBE, dried with Na₂SO₄, filtered and concentrated to afford the title compound. The title compound was used directly in next step as crude (69.7 g, 100% yield). [M+H] 247.

### Step 2: 2-allyl-6-bromo-4-chlorophenol

To 1-allyloxy-2-bromo-4-chloro-benzene (84.4 g, 341 mmol) was added mesitylene (170 mL). The reaction was purged with N₂ and heated at 170° C. with condenser attached (no running water) under argon (4-18 h). The reaction was allowed to cool to room temperature. Solvent was removed under reduced pressure to afford the title compound (73.3 g, 87% yield). [M+H] 247.

### Step 3: 2-bromo-4-chloro-6-(oxiran-2-ylmethyl)phenol

A solution of 2-allyl-6-bromo-4-chloro-phenol (29.3 g, 118 mmol) in DCM (237 mL) was cooled down to 0° C. using an ice bath. mCPBA (28.6 g, 124 mmol) was added portionwise. The reaction was allowed to warm to rt. The reaction was stirred overnight under room temperature. The reaction was quenched with 2M Na₂S₂O₃ solution (aq., 50 mL), diluted with water (100 mL), quenched with saturated aqueous sodium bicarbonate (200 mL). It was extracted DCM (2×100 mL), dried, filtered. Solvent was removed under reduced pressure to afford the title compound (33 g, 99% yield). [M+H] 263.

### Step 4: (7-bromo-5-chloro-2,3-dihydrobenzofuran-2-yl)methanol

To 2-bromo-4-chloro-6-(oxiran-2-ylmethyl)phenol (27.85 g, 105.68 mmol) in DCM (422.74 mL) at 0° C. was added TBSCl (19.11 g, 126.82 mmol) triethylamine (19.07 mL, 137.39 mmol) followed by DMAP (2.58 g, 21.14 mmol). The reaction was stirred at temperature for overnight. The reaction mixture was washed with water, dried over sodium sulfate, concentrated in vacuo. The crude residue was purified by flash column chromatography (silica gel, 0-10% EtOAc in hexanes) to afford the title compound (22.1 g, 55% yield). [M+H] 377.

### Step 5: (R)-(2-bromo-4-chloro-6-(oxiran-2-ylmethyl)phenoxy)(tert-butyl) Cimethylsilane

To a solution of (S,S)-Co(II)-salen (196.17 mg, 0.3200 mmol) in 3 mL toluene was added acetic acid (0.19 mL, 3.25 mmol). The mixture was stirred open to air for 30 min, then concentrated in vacuo. [2-bromo-4-chloro-6-(oxiran-2-ylmethyl)phenoxy]-tert-butyl-dimethyl-silane (9.98 mL, 32.48 mmol) was added to the residue followed by the addition of water (0.41 mL, 22.7 mmol). THF (7.3527 mL) was added until water all dissolves then capped with a glass stopper and let stir overnight. The solvent was removed under reduced pressure. The crude residue was purified by flash column chromatography (silica gel, 0-40% EtOAc in hexanes) to afford the title compound (4.15 g, 34% yield). [M+H] 377.

### Step 6: (R)-(7-bromo-5-chloro-2,3-dihydrobenzofuran-2-yl)methanol

To (R)-2-bromo-4-chloro-6-(oxiran-2-ylmethyl)phenoxy-tert-butyl-dimethyl-silane (4.15 g, 10.99 mmol) in THF (119.12 mL) at 0° C. was added TBAF (1M in THF) (13.18 mL, 13.18 mmol). The reaction mixture was stirred for 1 h and monitored by TLC. The reaction mixture was warmed to room temperature. Calcium carbonate (533.78 mg, 5.23 mmol), Amberlyst 16 (strongly acidic) (20 g) and methanol (20 mL) were added. The reaction mixture was continued stirring at room temperature for 1 h. The mixture was filtered over celite, washed with methanol, and concentrated in vacuo to afford the title compound (2.9 g, 100% yield). Crude HPLC showed 91% ee favoring second peak (4.6 min and 5.0 min, OZ-H column, 80-20 heptane/ethanol+0.1% DEA). [M+H] 263.

### Step 7: (R)-7-bromo-5-chloro-2,3-dihydrobenzofuran-2-carboxylic acid

A solution of (R)-(7-bromo-5-chloro-2,3-dihydrobenzofuran-2-yl)methanol (7.54 g, 28.6 mmol) in THF (76 mL) was cooled to 0° C. was added TEMPO (894 mg, 5.72 mmol), KBr (851 mg, 7.15 mmol), saturated sodium bicarbonate (15 mL) and bleach (80.5 mL, 85.8 mmol). The reaction mixture was gradually warmed to room temperature and stirred at room temperature for 2 h. The reaction mixture was diluted with 1M HCl to pH around 2. It was extracted with EtOAc (25 mL, 4×). Combined organics were dried, filtered and concentrated under reduced pressure. Solid was trituated with Et₂O/hexanes (10:1) to afford the title compound as white solids (3.9 g, 49% yield). [M+H] 277.

### Step 8: tert-butyl (R)-4-(7-bromo-5-chloro-2,3-dihydrobenzofuran-2-carbonyl)piperazine-1-carboxylate

To (R)-7-bromo-5-chloro-2,3-dihydrobenzofuran-2-carboxylic acid (4.16 g, 15 mmol), N-Boc-piperazine (3.07 g, 16.5 mmol) and methylimidazole (3.59 mL, 45 mmol) as a suspension in EtOAc (30 mL) was added T3P (17.86 mL, 30 mmol). The reaction mixture was stirred at room temperature (2-16 h). The reaction mixture was diluted with NaOH solution (1M, 150 mL), extracted with EtOAc, dried, filtered and concentrated in vacuo. The crude residue was purified by flash column chromatography (silica gel, 20-100% EtOAc in hexanes) to afford the title compound (5.4 g, 81% yield). [M+H] 445.

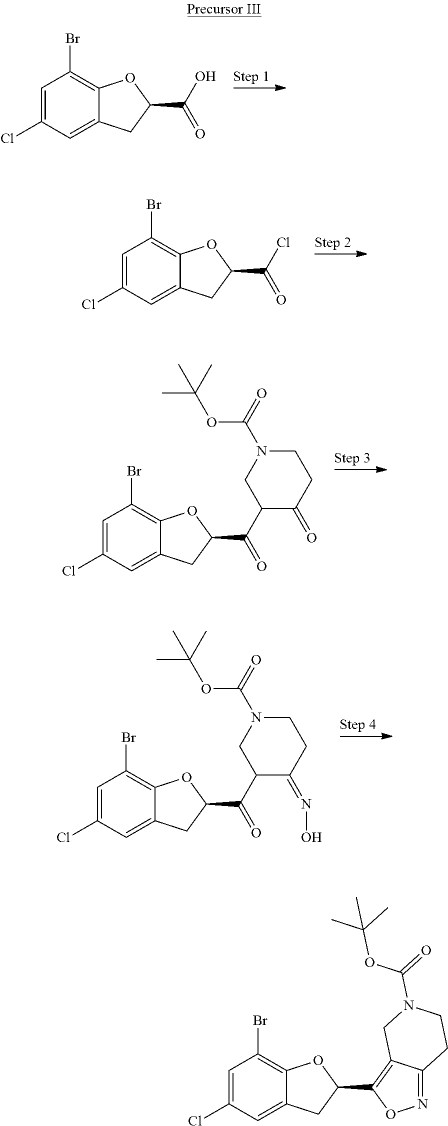 [SMILES: O=C(O)[C@H]1Cc2cc(Cl)cc(Br)c2O1 ; O=C(Cl)[C@H]1Cc2cc(Cl)cc(Br)c2O1 ; CC(C)(C)OC(=O)N1CCC(=O)C(C(=O)[C@H]2Cc3cc(Cl)cc(Br)c3O2)C1 ; CC(C)(C)OC(=O)N1CC/C(=N/O)C(C(=O)[C@H]2Cc3cc(Cl)cc(Br)c3O2)C1 ; CC(C)(C)OC(=O)N1CCc2noc([C@H]3Cc4cc(Cl)cc(Br)c4O3)c2C1]

### Step 1: (R)-7-bromo-5-chloro-2,3-dihydrobenzofuran-2-carbonyl Chloride

(R)-7-bromo-5-chloro-2,3-dihydrobenzofuran-2-carboxylic acid (precursor II, step 7) (2.77 g, 10 mmol) was dissolved in THF (50 mL) at 0° C. and oxalyl chloride (1.28 mL, 15 mmol) was added dropwise. The reaction mixture was stirred at rt for 1 h and concentrated under vacuum to give the title compound as a thick oil which was used in the next step without further purification. [M+H] 296.

### Step 2: tert-butyl 3-((R)-7-bromo-5-chloro-2,3-dihydrobenzofuran-2-carbonyl)-4-oxopiperidine-1-carboxylate

To a cooled solution (−78° C.) of tert-butyl 4-oxopiperidine-1-carboxylate (7.88, 39.62 mmol) in anhydrous THF (95 mL) was added a solution of LHMDS (39.6 mL, 1M in hexanes, 39.6 mmol). After stirring at −78° C. for 50 min, the reaction was added (R)-7-bromo-5-chloro-2,3-dihydrobenzofuran-2-carbonyl chloride (7.3 g, in 5 mL THF, 24.74 mmol), stirred at −78° C. for 50 min and then warmed to 0° C. The reaction was quenched with aqueous NH₄Cl at 0° C. and the aqueous layer was extracted with EtOAc. The combined organic layers were concentrated. The crude product was purified by flash column chromatography using 5-25% EtOAc/hexanes to give the title compound (3.5 g, 31%). [M+H] 458.

### Step 3: tert-butyl (Z)-3-((R)-7-bromo-5-chloro-2,3-dihydrobenzofuran-2-carbonyl)-4-(hydroxyimino)piperidine-1-carboxylate

tert-butyl 3-((R)-7-bromo-5-chloro-2,3-dihydrobenzofuran-2-carbonyl)-4-oxopiperidine-1-carboxylate (3.55 g, 7.75 mmol) was dissolved in EtOH (40 mL) and was added NH₂OH—HCl salt (802 mg, 11.63 mmol) and NaOAc (1.91 g, 13.25 mmol). The reaction was heated at 82° C. and stirred for 2 h. The reaction mixture was cooled and concentrated. The reaction mixture was diluted with water and extracted with EtOAc. The organic layers were combined and washed with NaHCO₃, concentrated to afford the title compound which was used as crude for the next step.

### Step 4: tert-butyl (R)-3-(7-bromo-5-chloro-2,3-dihydrobenzofuran-2-yl)-6,7-dihydroisoxazolo[4,3-c]pyridine-5(4H)-carboxylate

The crude tert-butyl (Z)-3-((R)-7-bromo-5-chloro-2,3-dihydrobenzofuran-2-carbonyl)-4-(hydroxyimino)piperidine-1-carboxylate (3.66 g, 7.75 mmol) was dissolved in DCM (40 mL) and was added MsCl (1.81 g, 15.9 mmol) followed by TEA (4.38 m1, 31 mmol). The reaction was stirred at RT for 2 h and concentrated. The reaction mixture was extracted with EtOAc, washed with 1M HCl, water, and brine. The organic layers were concentrated. The crude products were purified by chromatography (5-25% EtOAc/hexane) to afford the title compound (2.4 g, 68%). [M+H] 455.

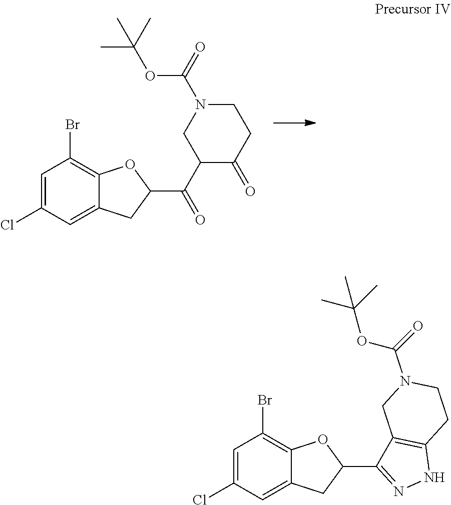 [SMILES: CC(C)(C)OC(=O)N1CCC(=O)C(C(=O)C2Cc3cc(Cl)cc(Br)c3O2)C1 ; CC(C)(C)OC(=O)N1CCc2[nH]nc(C3Cc4cc(Cl)cc(Br)c4O3)c2C1]

tert-Butyl-3-(7-bromo-5-chloro-2,3-dihydrobenzofuran-2-yl)-1,4,6,7-tetrahydro-5H-pyrazolo[4,3-c]pyridine-5-carboxylate. To a solution of tert-butyl-3-(7-bromo-5-chloro-2,3-dihydrobenzofuran-2-carbonyl)-4-oxopiperidine-1-carboxylate (60 mg, 0.13 mmol) in ethanol (2 mL) was added hydrazine monohydrate (0.04 mL, 3 eq). The mixture was heated to 60° C. for 3 h while stirring. The mixture was concentrated and extracted with EtOAc and H₂O. The organic layer was washed with brine, concentrated and dried to give crude product (60 mg). The pyrazole could be N-methylated with potassium carbonate and methyl iodide in DMF at room temperature and the N1 and N2 isomers separated by chromatography.

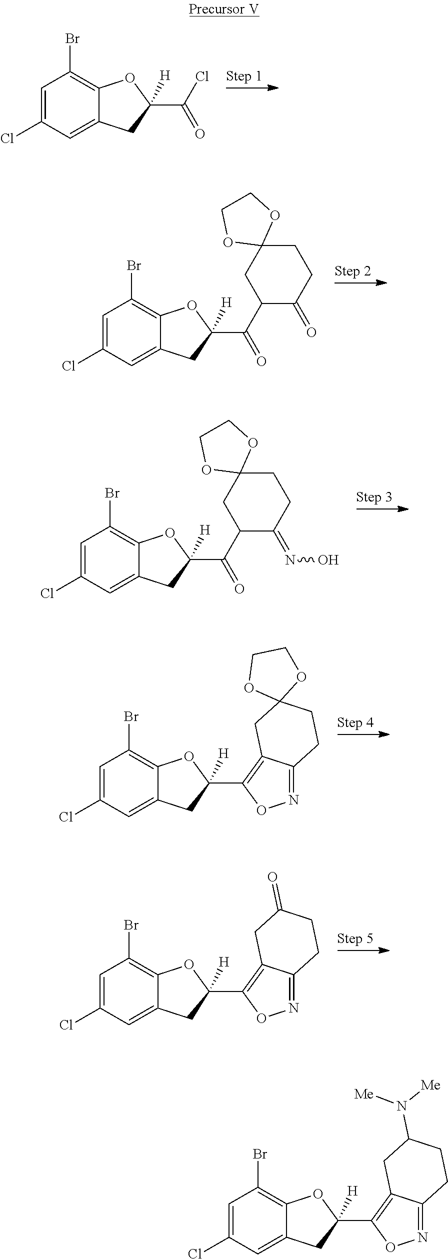 [SMILES: O=C(Cl)[C@@H]1Cc2cc(Cl)cc(Br)c2O1 ; O=C1CCC2(CC1C(=O)[C@@H]1Cc3cc(Cl)cc(Br)c3O1)OCCO2 ; O=C(C1CC2(CCC1=NO)OCCO2)[C@@H]1Cc2cc(Cl)cc(Br)c2O1 ; Clc1cc(Br)c2c(c1)C[C@@H](c1onc3c1CC1(CC3)OCCO1)O2 ; O=C1CCc2noc([C@@H]3Cc4cc(Cl)cc(Br)c4O3)c2C1 ; CN(C)C1CCc2noc([C@@H]3Cc4cc(Cl)cc(Br)c4O3)c2C1]

### Step 1

To a cooled solution (−78° C.) of 1,4-dioxaspiro[4.5]decan-8-one (26.18 g, 167.8 mmol) in anhydrous THF (220 mL) was added a solution of LiHMDS (168 ml, 1M in THF, 167.6 mmol). After stirring at −78° C. for 60 min, (R)-7-bromo-5-chloro-2,3-dihydrobenzofuran-2-carbonyl chloride was added (30.28 g, in 15 m1 THF, 102.6 mmol). The reaction mixture was stirred at −78° C. for 70 min and then warmed to 0° C. The reaction was quenched with aqueous NH₄Cl at 0° C. and the aqueous layer was extracted with EtOAc. The combined organic layers were washed with water, 0.5M HCl and brine, then concentrated. The crude product was purified with column chromatography using 5-25% EtOAc/hexane to give the title compound (31 g, 72.8%).

### Step 2

7-((R)-7-bromo-5-chloro-2,3-dihydrobenzofuran-2-carbonyl)-1,4-dioxaspiro[4.5]decan-8-one (16 g, 38.55 mmol) was dissolved in EtOH (150 mL) and NH₂OH—HCl salt (3.94 g, 56.92 mmol) was added followed by NaOAc-3H₂O (15.48 g, 114 mmol). The reaction was heated at 82° C. and stirred for 2 hrs. The reaction mixture was cooled and concentrated. The reaction mixture was diluted with water and extracted with EtOAc. The organic layers were combined and washed with NaHCO₃, concentrated. The title product was dried and used directly in the next step without further purification.

### Step 3

Crude ((R)-7-bromo-5-chloro-2,3-dihydrobenzofuran-2-yl)(8-(hydroxyimino)-1,4-dioxaspiro[4.5]decan-7-yl)methanone (18 g, 38.14 mmol) was dissolved in DCM (120 mL) and MsCl (5.88 m1, 76.28 mmol) was added followed by TEA (21.3 m1, 152.5 mmol). The reaction was stirred at room temperature for 2 hours and concentrated. The reaction mixture was extracted with EtOAc, washed with 1M HCl, water, and brine. The organic layers were concentrated. The crude products were purified by chromatography (5-25% EtOAc/hexane) to give the title compound (11.05 g 64%). [M+H]⁺ 455.

### Step 4

(R)-3-(7-bromo-5-chloro-2,3-dihydrobenzofuran-2-yl)-6,7-dihydro-4H-spiro[benzo[c]isoxazole-5,2′-[1,3]dioxolane] (13.97 g, 33.91 mmol) was dissolved in acetone (250 mL) and water (50 m1). p-Toluenesulfonic acid (23.3 g, 135.6 mmol) was added and the reaction was stirred at 60° C. for 4 hrs. The reaction mixture was cooled and concentrated. The mixture was diluted with water and extracted with EtOAc. The organic layer was washed with water, and brine. The crude product was concentrated and purified by chromatography (10-35% EtOAc/hexane) to give the title compound (9 g, 72%). [M+H]⁺ 368.

### Step 5

(R)-3-(7-bromo-5-chloro-2,3-dihydrobenzofuran-2-yl)-6,7-dihydrobenzo[c]isoxazol-5(4H)-one was dissolved in DCE (10 mL) and acetic acid (0.25 mL). dimethylamine (2M in THF, 6.7 mL, 13.3 mmol) and sodium triacetoxyborohydride (1.23 g, 5.78 mmol) were successively added at room temperature. The reaction mixture was stirred for 16 h and diluted with 1N NaOH and the crude product extracted with methyl ethyl ketone. Purification by silica gel chromatography with 0-10% MeOH in DCM gave the title compound (400 mg, 23%). [M+H]⁺ 493. Using this procedure other amines were installed onto the isoxazole framework.

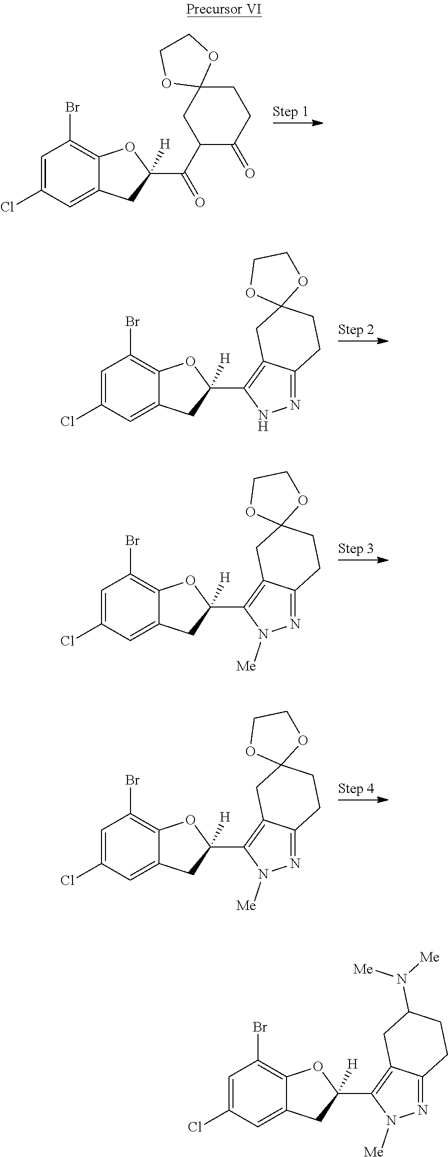 [SMILES: O=C1CCC2(CC1C(=O)[C@@H]1Cc3cc(Cl)cc(Br)c3O1)OCCO2 ; Clc1cc(Br)c2c(c1)C[C@@H](c1[nH]nc3c1CC1(CC3)OCCO1)O2 ; Cn1nc2c(c1[C@@H]1Cc3cc(Cl)cc(Br)c3O1)CC1(CC2)OCCO1 ; Cn1nc2c(c1[C@@H]1Cc3cc(Cl)cc(Br)c3O1)CC1(CC2)OCCO1 ; CN(C)C1CCc2nn(C)c([C@@H]3Cc4cc(Cl)cc(Br)c4O3)c2C1]

### Step 1

7-((R)-7-bromo-5-chloro-2,3-dihydrobenzofuran-2-carbonyl)-1,4-dioxaspiro[4.5]decan-8-one (10 g, 24.1 mmol) was dissolved in EtOH (100 mL) and was added anhydrous NH₂NH₂ (3.08 g, 96.38 mmol). The reaction was heated at 62° C. and stirred for 2 hrs. The reaction mixture was cooled and concentrated. The reaction mixture was diluted with water and extracted with EtOAc. The organic layers were combined and washed with NaHCO₃, concentrated. The title compound was dried to give a white solid (10 g, 98%).

### Step 2

(R)-3-(7-bromo-5-chloro-2,3-dihydrobenzofuran-2-yl)-2,4,6,7-tetrahydrospiro[indazole-5,2′-[1,3]dioxolane] (10 g, 24.33 mmol) was dissolved in dry DMF (55 mL) and was added MeI (4 m1, 60.8 mmol) followed by K₂CO₃ (10 g, 73 mmol). The reaction was stirred at room temperature for 24 hrs. The reaction mixture was quenched with water and extracted with EtOAc. The combined organic layers were washed with water and concentrated. The crude products were purified by chromatography (30-75% EtOAc/Hexane) to give the title compound (2.79 g, 27%).

### Step 3

(R)-3-(7-bromo-5-chloro-2,3-dihydrobenzofuran-2-yl)-2-methyl-2,4,6,7-tetrahydrospiro[indazole-5,2′-[1,3]dioxolane] (2.79 g, 6.56 mmol) was dissolved in acetone (50 mL) and water (10 m1). p-Toluenesulfonic acid (4.52 g, 26.26 mmol) was added and the reaction was stirred at 60° C. for 4 hrs. The reaction mixture was cooled and concentrated. The mixture was diluted with water and extracted with EtOAc. The organic layer was washed with water, and brine. The crude product was concentrated and purified by chromatography (20-85% EtOAc/hexane) to give the title compound (1.6 g, 64%). [M+H]⁺ 381.

### Step 4

To a solution of 3-[(2R)-7-bromo-5-chloro-2,3-dihydrobenzofuran-2-yl]-2-methyl-6,7-dihydro-4H-indazol-5-one (518 mg, 1.36 mmol) and sodium triacetoxyborohydride (575.29 mg, 2.71 mmol) in ethanol (2 mL) was added dimethylamine (2M in THF, 2.71 mL, 5.43 mmol). The resulting mixture was heated to 80° C. for overnight. The reaction mixture was allowed to cool to room temperature and diluted with EtOAc, washed with water, brine and dried over sodium sulfate. The solvent was removed and the crude residue was purified by column chromatography eluting with 0-20% MeOH/DCM to afford a light tan powder as the title product (421.1 mg, 75% yield). Using this procedure other amines were installed onto the pyrazole framework.

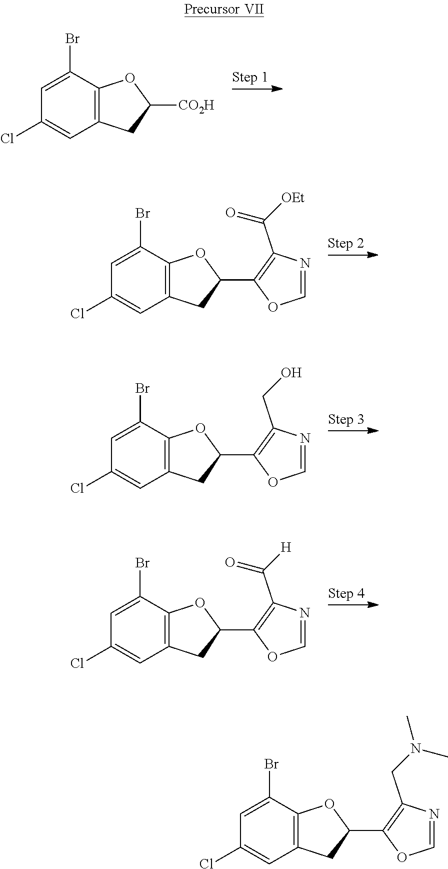 [SMILES: O=C(O)[C@H]1Cc2cc(Cl)cc(Br)c2O1 ; CCOC(=O)c1ncoc1[C@H]1Cc2cc(Cl)cc(Br)c2O1 ; OCc1ncoc1[C@H]1Cc2cc(Cl)cc(Br)c2O1 ; O=Cc1ncoc1[C@H]1Cc2cc(Cl)cc(Br)c2O1 ; CN(C)Cc1ncoc1[C@H]1Cc2cc(Cl)cc(Br)c2O1]

### Step 1

ethyl 5-[(2R)-7-bromo-5-chloro-2,3-dihydrobenzofuran-2-yl]oxazole-4-carboxylate. To compound 14 (9.75 g, 35.2 mmol) and K₂CO₃ (19.9 g, 144.1 mmol) in DMF (55 m1) at 0° C. was added diphenylphosphoryl azide (10.1 m1, 46.8 mmol) and ethyl isocyanoacetate (7.9 m1, 72.1 mmol). The reaction was stirred at RT overnight. The reaction mixture was diluted with water, and extracted with EtOAc. The organic layers were washed with water (3×), and brine. The crude product was concentrated and purified with column chromatography using 20-50% EtOAc/hexane to give ethyl 5-[(2R)-7-bromo-5-chloro-2,3-dihydrobenzofuran-2-yl]oxazole-4-carboxylate (4.4 g, 33%). [M+H]=372.

### Step 2: [5-[(2R)-7-bromo-5-chloro-2,3-dihydrobenzofuran-2-yl]oxazol-4-yl]methanol

To a vial of ethyl 5-[(2R)-7-bromo-5-chloro-2,3-dihydrobenzofuran-2-yl]oxazole-4-carboxylate (3.19 g, 8.9 mmol) in 10:1 THF:water (88 mL) was added sodium borohydride (1.68 g, 44.5 mmol) and stirred for 48 h. The reaction mixture was diluted with water (50 mL) then extracted with EtOAc (3×30 mL). Dried over anhydrous MgSO₄, filtered, and concentrated in vacuo. The crude product was purified by flash chromatography (0-100% EtOAc) to give [5-[(2R)-7-bromo-5-chloro-2,3-dihydrobenzofuran-2-yl]oxazol-4-yl]methanol (1.48 g, 50% yield). [M+H]=329.9.

### Step 3: 5-[(2R)-7-bromo-5-chloro-2,3-dihydrobenzofuran-2-yl]oxazole-4-carbaldehyde

Weighed [5-[(2R)-7-bromo-5-chloro-2,3-dihydrobenzofuran-2-yl]oxazol-4-yl]methanol (566 mg, 1.72 mmol) and dissolved in DCM (5.7 mL). To the reaction mixture was added NaHCO₃ (288 mg, 3.42 mmol) then Dess-Martin Periodinane (840 mg, 1.90 mmol). The reaction was monitored by TLC and stirred for 40 min. The reaction mixture was filtered to remove the solids then concentrated in vacuo. The crude product was purified by flash, (0-60% EtOAc in hexanes) to give 5-[(2R)-7-bromo-5-chloro-2,3-dihydrobenzofuran-2-yl]oxazole-4-carbaldehyde (136 mg, 91% yield). [M+H]=327.9

### Step 4: 1-[5-[(2R)-7-bromo-5-chloro-2,3-dihydrobenzofuran-2-yl]oxazol-4-yl]-N,N-dimethyl-methanamine

Weighed 5-[(2R)-7-bromo-5-chloro-2,3-dihydrobenzofuran-2-yl]oxazole-4-carbaldehyde (30 mg, 0.09 mmol) and dissolved in DCE (0.46 mL). Added dimethyl amine solution (0.23 mL, 0.46 mmol, 2 M in THF) followed by sodium triacetoxyborohydride (39 mg, 0.18 mmol). The reaction was monitored by LCMS until starting material was consumed. The reaction was quenched with with 1 mL of 1M NaOH (aq.) and the aqueous phase was extracted with DCM (3×3 mL). The combined organic extracts was dried over anhydrous MgSO₄, filtered, and concentrated in vacuo. The crude product was purified by flash chromatography (0-10% MeOH in DCM) to give 1-[5-[(2R)-7-bromo-5-chloro-2,3-dihydrobenzofuran-2-yl]oxazol-4-yl]-N,N-dimethyl-methanamine (15 mg, 47% yield). [M+H]=357.0. Using this procedure other amines were installed onto the oxazole framework.

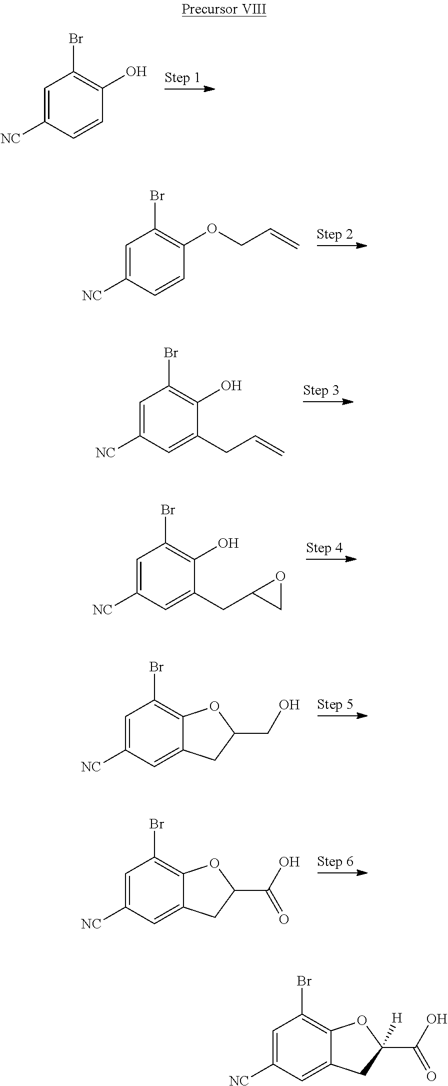

### Step 1

To a solution of 3-bromo-4-hydroxybenzylnitrile (234 g, 1.18 mol) in acetone (800 mL) was added allyl bromide (150 g) and K₂CO₃ (180 g). The mixture was stirred at 60° C. for 6 hr. The mixture was filtered, washed with acetone 200 mL. The organic layer was dried and concentrated to give the crude product as colorless oil (280 g).

### Step 2

A solution of 4-(allyloxy)-3-bromobenzonitrile (280 g) in mesitylene (100 mL) was heated to 180° C. for 48 hr. The mixture was purified with flash column (0-20% EtOAc in hexane) to give the title compound (107 g).

### Step 3

To a solution of 3-allyl-5-bromo-4-hydroxybenzonitrile (21 g) in DCM (400 mL) was added mCPBA (21.5 g). The mixture was stirred at room temperature overnight. The 2^{nd} batch was a much slower reaction. The first batch reaction was completed. The mixture was filtered off the solid, washed with saturated NaHCO₃, NaS₂O₃ and brine. The organic layer was dried and concentrated to give the title compound (16.4 g).

### Step 4

3-bromo-4-hydroxy-5-(oxiran-2-ylmethyl)benzonitrile (16.4 g, 64.5 mmol) was dissolved in MeOH (200 mL) at 0° C. and treated with KOH (3.98 g), followed by H₂O (20 mL). The reaction was stirred at 0° C. for 1 hour. The mixture was neutralized with HCl (6 N, 10 mL) and filtered to give crude product (16.4 g).

### Step 5

To a solution of 7-bromo-2-(hydroxymethyl)-2,3-dihydrobenzofuran-5-carbonitrile (16 g, 63 mmol) in THF (100 mL) and saturated NaHCO₃ (100 mL) at 0° C. was added TEMPO (2 g, 0.2 eq), potassium bromide (1.5 g, 0.2 eq), and NaClO (12% aq. solution, 120 mL, 3 eq) drop wise with additional funnel while stirring. The mixture was warmed to room temperature slowly. After 4 hour, the mixture was concentrated in vacuo to remove most THF, charged with 40 mL Na₂CO₃ solution, filtered and washed with MTBE (50 mL×2). The solid was dissolved in 1N HCl, extracted with EtOAc to give the title compound (7 g).

### Step 6

To a solution of 7-bromo-5-cyano-2,3-dihydrobenzofuran-2-carboxylic acid (7 g, 26 mmol) in MeOH (75 mL) was added (S)-(−)-(1-naphtyl)-ethylamine (4.5 g, 1 eq) drop wise while stirring. The reaction was stirred for 30 min. The crude was concentrated and dried under high vacuum to give a foamy solid. The crude material was recrystallized 3 times in EtOH to provide the title compound.

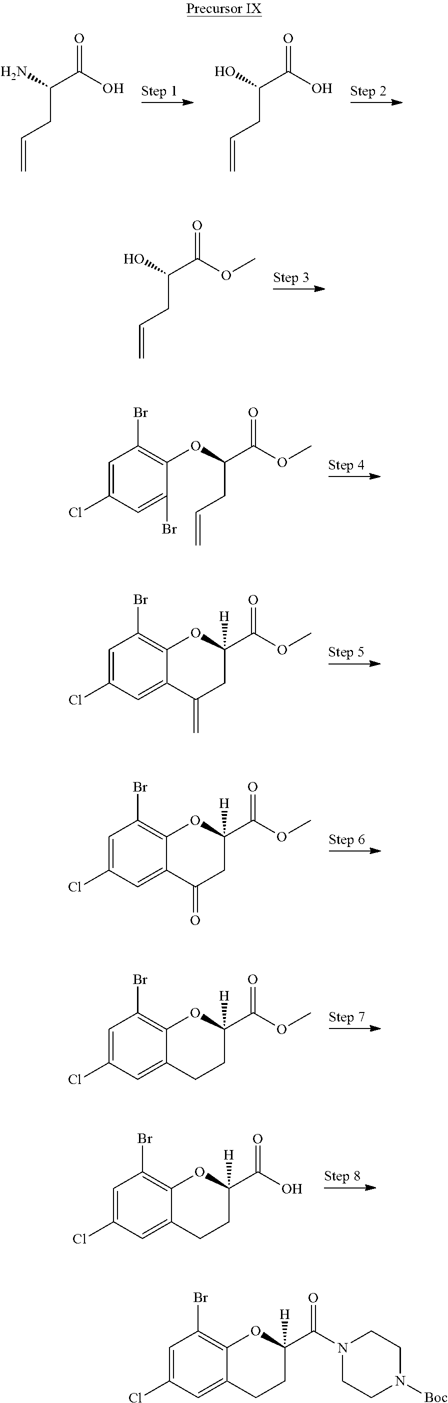

### Step 1: (S)-2-hydroxypent-4-enoic Acid

A solution of (S)-2-aminopent-4-enoic acid (24 g, 205 mmol) in 1.75 L H₂O and 437.5 mL AcOH (4:1 v/v) was cooled to 0° C. To the solution was added an aqueous solution (300 mL) of sodium nitrite (29.25 g, 410 mmol) dropwise via an addition funnel over 4 hours. The mixture was further stirred for 2 hours at 0° C., and then slowly warm to room temperature over 16 hours. At this point the mixture was again placed at 0° C. and quenched by addition of 217.5 mL methylamine (2.0 M in THF, 435 mmol). The solution was then acidified to pH 2 by adding 6 M HCl, and extracted with ethyl acetate. The combined organic layers were dried (Na₂SO₄), concentrated in vacuo, and the crude residue was purified by flash chromatography 10% MeOH/DCM, 22.6 g (S)-2-hydroxypent-4-enoic acid was obtained (94%). Reference: Organic Letters, 17(4), 968-971; 2015

### Step 2: methyl (S)-2-hydroxypent-4-enoate

A solution of (S)-2-hydroxypent-4-enoic acid (452 mg) and H₂SO₄ (2 drops) in MeOH (4 mL) was stirred overnight at room temperature. After concentration, the solution was diluted with ethyl acetate, and then extracted with saturated NaHCO₃ in water. The combined organic layers were dried over Na₂SO₄, filtered, and concentrated in vacuo to give the desired product which was used without further purification.

### Step 3: methyl (R)-2-(2,6-dibromo-4-chlorophenoxy)pent-4-enoate

To a solution of DIAD (0.61 mL, 3.1 mmol) and triphenylphosphine (817 mg, 3.1 mmol) in THF (4.0 mL) at 0° C. was added a solution of methyl (2S)-2-hydroxypent-4-enoate (506 mg, 3.9 mmol) and 2,6-dibromo-4-chloro-phenol (743 mg, 2.59 mmol) in THF (1 mL). The resulting solution was stirred at room temperature over 48 hours then concentrated in vacuo. The residue was then suspended in 20% diethyl ether/Hexanes, stirred for two hours, filtered, and concentrated in vacuo. The crude residue was then purified by column chromatography 2% EtOAc/Hexanes to give the desired product 796 mg (52%). [M+H=398.8]

### Step 4: methyl (R)-8-bromo-6-chloro-4-methylenechromane-2-carboxylate

A solution of methyl (R)-2-(2,6-dibromo-4-chlorophenoxy)pent-4-enoate (796 mg, 2.0 mmol), potassium acetate (986 mg, 10.0 mmol), dppe (318 mg, 0.8 mmol) and Pd(OAc)₂ (89 mg, 0.4 mmol) in DMF (6 mL) under a nitrogen atmosphere was stirred at 90° C. for 16 hours. After cooling to room temperature, the mixture was diluted with ethyl acetate and washed with saturated sodium chloride in water. The combined organic layers were dried with Na₂SO₄, filtered, concentrated in vacuo, and purified by column chromatography using 0-20% EtOAc/Hexanes to give the desired product 263 mg (41%). [M+H=317.0]

### Step 5: methyl (R)-8-bromo-6-chloro-4-oxochromane-2-carboxylate

Into a solution of methyl (R)-8-bromo-6-chloro-4-methylenechromane-2-carboxylate (263 mg, 0.8 mmol) in DCM (10 mL) at −78° C. was bubbled O₃ (g) for 15 minutes then flushed with Ar(g). PPh₃ (870 mg, 3.3 mmol) was then added and the resulting mixture was slowly warmed to room temperature over 16 hours and concentrated in vacuo. the crude residue was purified by flash chromatography (0-10% EtOAc in hexanes) to give the desired product 89 mg (35%). [M+H=318.9]

### Step 6: methyl (R)-8-bromo-6-chlorochromane-2-carboxylate

A solution of methyl (R)-8-bromo-6-chloro-4-oxochromane-2-carboxylate (89 mg, 0.29 mmol) and triethylsilane (0.18 mL, 1.12 mmol) in TFA (0.43 mL, 5.59 mmol) was heated for 16 hours at 50° C. in a sealed tube. The mixture was then cooled, concentrated in vacuo and purified by column chromatography (0-10% EtOAc in hexanes) to give the desired product 44 mg (50%). [M+H=326.9]

### Step 7: (R)-8-bromo-6-chlorochromane-2-carboxylic Acid

To a solution of methyl (2R)-8-bromo-6-chloro-chromane-2-carboxylate (100 mg, 0.33 mmol) in MeOH (0.5 mL) and THF (1.0 mL) was added lithium hydroxide (1.31 mL, 0.33 mmol) at room temperature. The solution was left to stir at room temperature for 4 hours, evaporated in vacuo, treated with 3M hydrochloric acid in water, and diluted with EtOAc. The combined organic phases were dried over Na₂SO₄, filtered, and evaporated in vacuo to give the pure acid without further purification. (2R)-8-bromo-6-chloro-chromane-2-carboxylic acid 90 mg (94%). [M+Na=314.9]

### Step 8: tert-butyl (R)-4-(8-bromo-6-chlorochromane-2-carbonyl)piperazine-1-Carboxylate

To (2R)-8-bromo-6-chloro-chromane-2-carboxylic acid (48 mg, 0.16 mmol), N-Boc-piperazine (34 mg, 0.18 mmol) and pyridine (0.07 mL, 0.82 mmol) in ethyl acetate (2 mL) was added 1-propanephosphonic acid cyclic anhydride (0.19 mL, 0.33 mmol). Stirred overnight, diluted with EtOAc then NaOH solution (1M, until basic). Extracted with EtOAc, dried, filtered and concentrated. Flash chromatography with 0-->100% EtOAc/hex gave the title compound.

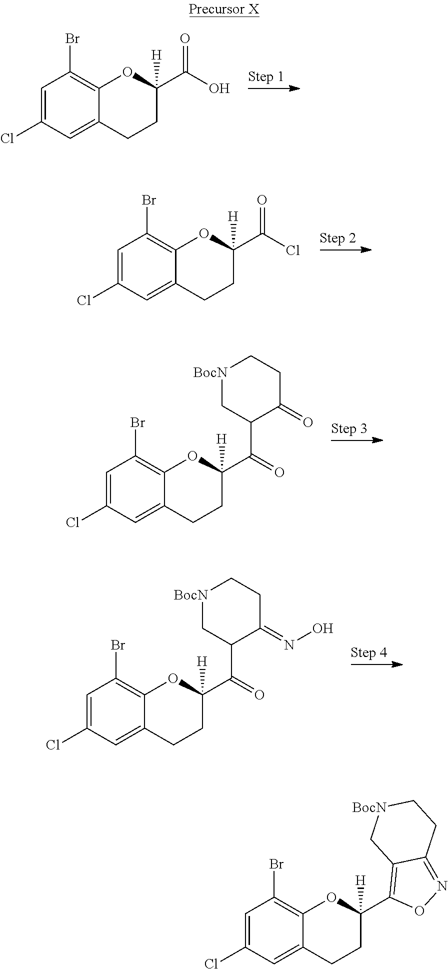 [SMILES: O=C(O)[C@H]1CCc2cc(Cl)cc(Br)c2O1 ; O=C(Cl)[C@H]1CCc2cc(Cl)cc(Br)c2O1 ; CC(C)(C)OC(=O)N1CCC(=O)C(C(=O)[C@H]2CCc3cc(Cl)cc(Br)c3O2)C1 ; CC(C)(C)OC(=O)N1CC/C(=N\O)C(C(=O)[C@H]2CCc3cc(Cl)cc(Br)c3O2)C1 ; CC(C)(C)OC(=O)N1CCc2noc([C@H]3CCc4cc(Cl)cc(Br)c4O3)c2C1]

### Step 1: (R)-8-bromo-6-chlorochromane-2-carbonyl Chloride

To a solution of (2R)-8-bromo-6-chloro-chromane-2-carboxylic acid (400 mg, 1.37 mmol) and DMF (3 drops) in dichloromethane (6.9 mL) was added (COCl)₂ (0.24 mL, 2.7 mmol) at 0° C. The solution was left to slowly warm to room temperature over 16 hours, then concentrated in vacuo, and the product 424 mg (99%) was used without further purification.

### Step 2: tert-butyl 3-((R)-8-bromo-6-chlorochromane-2-carbonyl)-4-oxopiperidine-1-Carboxylate

To a solution of Boc-4-piperidone (273.0 mg, 1.37 mmol) in THF (4.6 mL) at −78 OC was added LiHMDS (1.37 mL, 1.37 mmol, 1.0 M solution in THF), and the solution was left to stir at −78° C. for 30 minutes. After this point a solution of (2R)-8-bromo-6-chloro-chromane-2-carbonyl chloride (424 mg, 1.37 mmol) in THF (2.0 mL) was added to the solution of ketone at −78° C. and the mixture was stirred for 30 minutes at this temperature, then warmed to 0° C. and left to stir for one hour. The reaction was then quenched with saturated NH₄Cl in water at 0° C. and the aqueous layer was extracted with EtOAc. The combined organic layers were concentrated in vacuo then purified with column chromatography (5-25% EtOAc in hexane) to give the desired product tert-butyl 3-[(2R)-8-bromo-6-chloro-chromane-2-carbonyl]-4-oxo-piperidine-1-carboxylate (260 mg, 40% yield). [M+Na=494.0].

### Step 3: tert-butyl (E)-3-((R)-8-bromo-6-chlorochromane-2-carbonyl)-4-(hydroxyimino)piperidine-1-carboxylate

To a solution of tert-butyl 3-[(2R)-8-bromo-6-chloro-chromane-2-carbonyl]-4-oxo-piperidine-1-carboxylate (260 mg, 0.5500 mmol), NaOAc (135.29 mg, 1.65 mmol), and water (29.7 uL, 1.65 mmol) in Ethanol (2.75 mL) at room temperature was added NH₂OH—HCl (49.68 mg, 0.7100 mmol). The solution was then heated to 80 C and left to stir for 4 hours. The reaction mixture was then cooled and concentrated in vacuo. The reaction mixture was quenched with water and extracted with EtOAc. The organic layers were concentrated and the crude products were purified by chromatography (25-65% EtOAc/hexane) to give the desired product tert-butyl (4Z)-3-[(2R)-8-bromo-6-chloro-chromane-2-carbonyl]-4-hydroxyimino-piperidine-1-carboxylate 220 mg (82%). [M+H=487.0].

### Step 4: tert-butyl (R)-3-(8-bromo-6-chlorochroman-2-yl)-6,7-dihydroisoxazolo[4,3-c]pyridine-5 (4H)-carboxylate

To a solution of tert-butyl (4Z)-3-[(2R)-8-bromo-6-chloro-chromane-2-carbonyl]-4-hydroxyimino-piperidine-1-carboxylate (220.0 mg, 0.45 mmol) and triethylamine (0.25 mL, 1.8 mmol) in dichloromethane (2.75 mL) at room temperature was added MsCl (70 μL, 0.90 mmol) dropwise. The reaction was stirred at room temperature for 4 hours and concentrated in vacuo. The reaction mixture was then diluted with water, extracted with EtOAc and the combined organic layers were dried over sodium sulfate, filtered and concentrated in vacuo. The crude products were purified by flash chromatography (25-65% EtOAc/hexane) to give the desired product 180 mg (85%). [M+Na=490.9].

### General Procedure A

### Amide Formation of 7-bromo-5-chloro-2,3-dihydrobenzofuran-2-carboxylic Acid

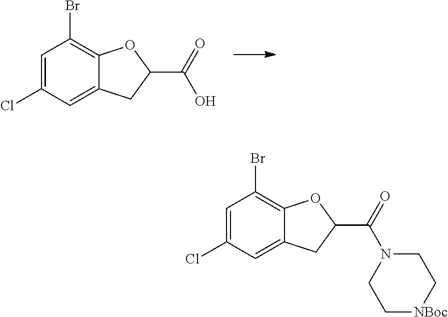 [SMILES: O=C(O)C1Cc2cc(Cl)cc(Br)c2O1 ; CC(C)(C)OC(=O)N1CCN(C(=O)C2Cc3cc(Cl)cc(Br)c3O2)CC1]

To a solution of 7-bromo-5-chloro-2,3-dihydrobenzofuran-2-carboxylic acid (39 mg, 0.14 mmol), amine (0.28 mmol), and methylimidazole (35 mg, 0.42 mmol) in EtOAc/DMF (1:2, 0.5/1 mL) was added T3P (179 mg, 0.28 mmol). The resulting reaction was stirred at room temperature (2-16 h). The reaction mixture was stirred at room temperature (2-16 h). The reaction mixture was diluted with NaOH solution (1M, 1.5 mL), extracted with EtOAc, dried, filtered and concentrated in vacuo. The crude residue was purified by flash column chromatography (silica gel, 0-100% EtOAc in hexanes or 0-10% MeOH/DCM) to afford the title compound (55.9 mg, 90% yield). [M+H] 445.

For instance when T3P didn't work, it was replaced with HATU (1.2 eq) and DIEA (3.2 eq) as base and DMF as solvent.

### General Procedure B

### Stille Cross Coupling of tert-butyl 4-(7-bromo-5-chloro-2,3-dihydrobenzofuran-2-carbonyl)piperazine-1-carboxylate

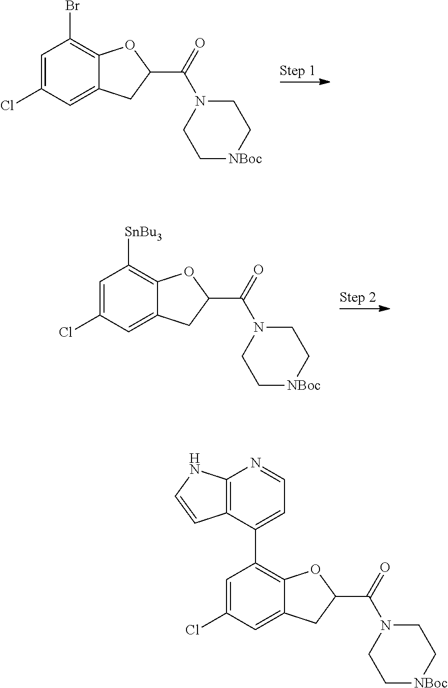

### Step 1: tert-butyl 4-(5-chloro-7-(tributylstannyl)-2,3-dihydrobenzofuran-2-carbonyl)piperazine-1-carboxylate

A solution of tert-butyl 4-(7-bromo-5-chloro-2,3-dihydrobenzofuran-2-carbonyl)piperazine-1-carboxylate (3.0 g, 6.73 mmol) and Bis(Triphenylphosphine)palladium (II) chloride (236 mg, 0.337 mmol) in dioxane (67 mL) was evacuated/backfilled with argon (3×). To this mixture was added tributyl(tributylstannyl)stannane (5.86 g, 10.1 mmol). The resulting mixture was stirred at 110° C. for (6-18 h). Reaction mixture was allowed to cool to room temperature and solvent was removed under reduced pressure. The crude residue was purified by flash column chromatography (silica gel, 0-50% EtOAc in hexanes) to afford the title compound (3.2 g, 72% yield) as clear oil. [M+H] 657.

### Step 2: tert-butyl 4-(5-chloro-7-(1H-pyrrolo[2,3-b]pyridin-4-yl)-2,3-dihydrobenzofuran-2-carbonyl)piperazine-1-carboxylate

To tert-butyl 4-[5-chloro-7-tributylstannyl-2,3-dihydrobenzofuran-2-carbonyl]piperazine-1-carboxylate (150 mg, 0.23 mmol), PdCl₂(PPh₃)₂ (16.05 mg, 0.02 mmol), 4-chloro-1H-pyrrolo[2,3-b]pyridine (35 mg, 0.23 mmol) and copper oxide (33 mg, 0.23 mmol) was added 1,4-Dioxane (1.5 mL). The resulting mixture was spared with argon for 5 min and heated to 100° C. with stirring for 6 h. The solvent was removed under reduced pressure and the crude residue was purified by flash column chromatography (silica gel, 20-100% EtOAc in hexanes) to afford the title compound (66.7 mg, 60%). [M+H] 483.

### General Procedure C

### Suzuki Cross Coupling of tert-butyl 4-(7-bromo-5-chloro-2,3-dihydrobenzofuran-2-carbonyl)piperazine-1-carboxylate

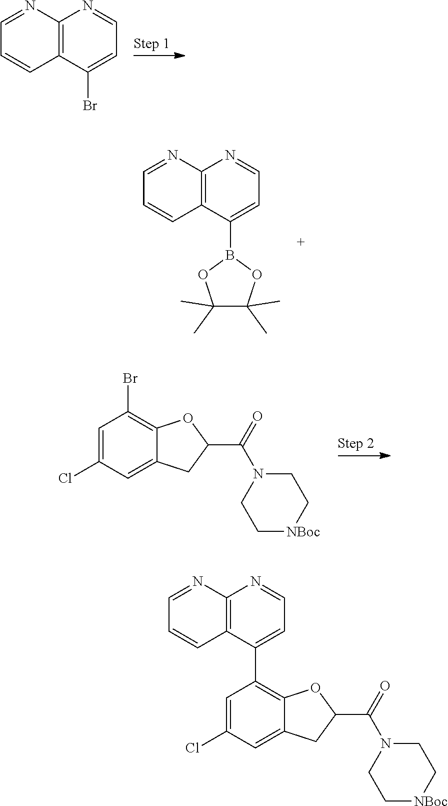 [SMILES: Brc1ccnc2ncccc12 ; CC1(C)OB(c2ccnc3ncccc23)OC1(C)C ; CC(C)(C)OC(=O)N1CCN(C(=O)C2Cc3cc(Cl)cc(Br)c3O2)CC1 ; CC(C)(C)OC(=O)N1CCN(C(=O)C2Cc3cc(Cl)cc(-c4ccnc5ncccc45)c3O2)CC1]

### Step 1: 4-(4,4,5,5-tetramethyl-1,3,2-dioxaborolan-2-yl)-1,8-naphthyridine

To 4-bromo-1,8-naphthyridine (125 mg, 0.66 mmol), Bis(pinacolato)diboron (160 mg, 0.6 mmol), PdCl₂dppf (44 mg, 0.06 mmol), AcOK (176 mg, 1.8 mmol) was added 1,4-Dioxane (3 mL). The resulting mixture was heated to 100° C. with stirring for 3 h. The reaction mixture was allowed to warm to room temperature and diluted with water (5 mL), extracted with EtOAc (2×), dried over sodium sulfate, filtered, and concentrated under reduced pressure to afford the title compound which was used directly for the next step (551 mg, 100% yield). [M+H] 257.

### Step 2: tert-butyl 4-(5-chloro-7-(1,8-naphthyridin-4-yl)-2,3-dihydrobenzofuran-2-carbonyl)piperazine-1-carboxylate

To tert-butyl 4-(7-bromo-5-chloro-2,3-dihydrobenzofuran-2-carbonyl)piperazine-1-carboxylate (281 mg, 0.60 mmol), 4-(4,4,5,5-tetramethyl-1,3,2-dioxaborolan-2-yl)-1H-pyrrolo[2,3-b]pyridine (154 mg, 0.60 mmol), and PdCl₂(PPh₃)₂ (42.1 mg, 0.06 mmol) was added toluene (1.8 mL), ethanol (1.8 mL), and aq. sodium carbonate (1M) (1.8 mL, 1.8 mmol). The resulting mixture was stirred vigorously at 100° C. (2-16 h). The reaction mixture was warmed to room temperature, diluted with H₂O (2 mL). The mixture was extracted with EtOAc (2×). Organic layers were combined, dried and concentrated in vacuo. The crude residue was purified by flash column chromatography (silica gel, 10-100% EtOAc in hexanes) to afford the title compound (71 mg, 24% yield). [M+H] 496.

### General Procedure D

### Deprotection of tert-butyl 4-(5-chloro-7-(pyridin-4-yl)-2,3-dihydrobenzofuran-2-carbonyl)piperazine-1-carboxylate

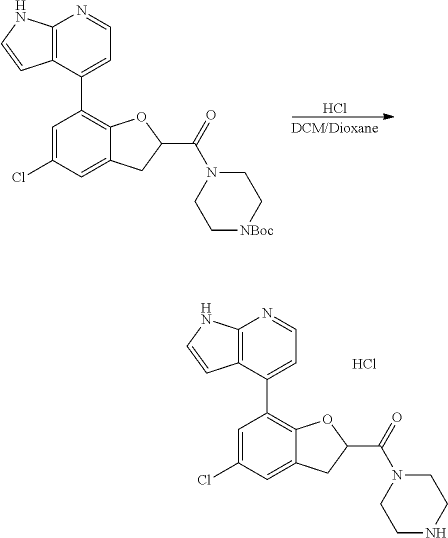 [SMILES: CC(C)(C)OC(=O)N1CCN(C(=O)C2Cc3cc(Cl)cc(-c4ccnc5[nH]ccc45)c3O2)CC1 ; Cl ; O=C(C1Cc2cc(Cl)cc(-c3ccnc4[nH]ccc34)c2O1)N1CCNCC1 ; Cl]

tert-butyl 4-(5-chloro-7-(pyridin-4-yl)-2,3-dihydrobenzofuran-2-carbonyl)piperazine-1-carboxylate (338 mg, 0.7 mmol) was dissolved in 2 mL of DCM and 2 mL of 4M solution of HCl in dioxane was added to it. Resulting mixture was stirred at room temperature until complete conversion was reached (1-3 h). After that solvents were removed in vacuo and residue was dried on high vacuum for 12 h to afford the title compound (268 mg, 100% yield). [M+H] 383.

### General Procedure E

### Deprotection of tert-butyl 4-(5-chloro-7-(pyridin-4-yl)-2,3-dihydrobenzofuran-2-carbonyl)piperazine-1-carboxylate

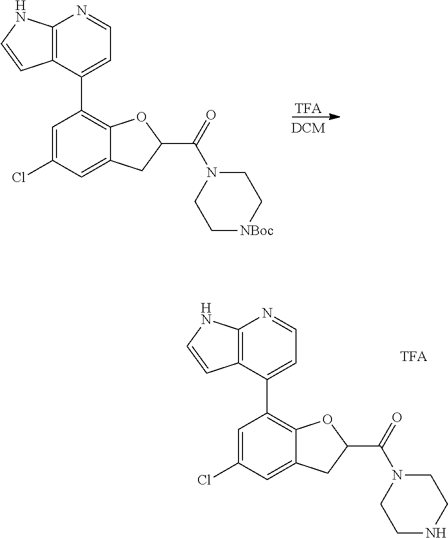 [SMILES: CC(C)(C)OC(=O)N1CCN(C(=O)C2Cc3cc(Cl)cc(-c4ccnc5[nH]ccc45)c3O2)CC1 ; O=C(C1Cc2cc(Cl)cc(-c3ccnc4[nH]ccc34)c2O1)N1CCNCC1 ; "TFA" ; "TFA"]

tert-butyl 4-(5-chloro-7-(pyridin-4-yl)-2,3-dihydrobenzofuran-2-carbonyl)piperazine-1-carboxylate (38.56 mg, 0.08 mmol) was treated with TFA/DCM (1:1, 1 mL). The resulting mixture was stirred at room temperature until complete conversion was reached (1-3 h). The solvents were removed in vacuo and residue was dried on high vacuum to afford the title compound (30.56 mg, 100% yield). [M+H] 383.

### General Procedure F

### Freebase of (5-chloro-7-(1H-pyrrolo[2,3-b]pyridin-4-yl)-2,3-dihydrobenzofuran-2-yl)(piperazin-1-yl)methanone hydrochloride or (5-chloro-7-(1H-pyrrolo[2,3-b]pyridin-4-yl)-2,3-dihydrobenzofuran-2-yl)(piperazin-1-yl)methanone compound with 2,2,2-trifluoro-1l3-ethan-1-one (1:1)

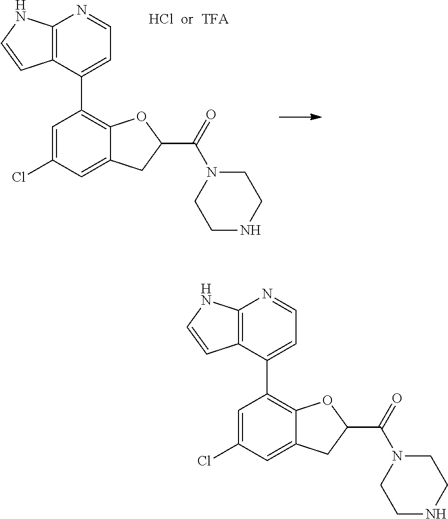 [SMILES: O=C(C1Cc2cc(Cl)cc(-c3ccnc4[nH]ccc34)c2O1)N1CCNCC1 ; Cl ; O=C(C1Cc2cc(Cl)cc(-c3ccnc4[nH]ccc34)c2O1)N1CCNCC1]

The (5-chloro-7-(1H-pyrrolo[2,3-b]pyridin-4-yl)-2,3-dihydrobenzofuran-2-yl)(piperazin-1-yl)methanone hydrochloride or (5-chloro-7-(1H-pyrrolo[2,3-b]pyridin-4-yl)-2,3-dihydrobenzofuran-2-yl)(piperazin-1-yl)methanone compound (1:1) was dissolved in MeOH/DCM (20/80, 2 mL) and filtered through a PL-HCO3 MP SPE tube. The solvent was removed under reduced vacuum to afford the title compound as a free base. [M+H] 383.

### General Procedure G

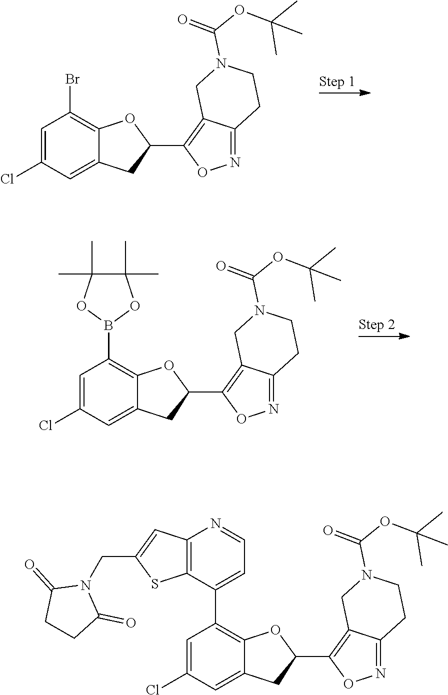 [SMILES: CC(C)(C)OC(=O)N1CCc2noc([C@H]3Cc4cc(Cl)cc(Br)c4O3)c2C1 ; CC(C)(C)OC(=O)N1CCc2noc([C@H]3Cc4cc(Cl)cc(B5OC(C)(C)C(C)(C)O5)c4O3)c2C1 ; CC(C)(C)OC(=O)N1CCc2noc([C@H]3Cc4cc(Cl)cc(-c5ccnc6cc(CN7C(=O)CCC7=O)sc56)c4O3)c2C1]

### Step 1: tert-Butyl (R)-3-(5-chloro-7-(4,4,5,5-tetramethyl-1,3,2-dioxaborolan-2-yl)-2,3-dihydrobenzofuran-2-yl)-6,7-dihydroisoxazolo[4,3-c]pyridine-5(4H)-carboxylate

A mixture of tert-butyl 3-(7-bromo-5-chloro-2,3-dihydrobenzofuran-2-yl)-6,7-dihydro-4H-isoxazolo[4,3-c]pyridine-5-carboxylate (2.0 g, 4.39 mmol), bis(pinacolato)diboron (1.39 g, 5.49 mmol), potassium acetate (0.54 g, 5.49 mmol), and 1,1′-bis(diphenylphosphino)ferrocene-palladium(II) dichloride (0.18 g, 0.24 mmol) in 20 mL dioxane was heated to 100° C. for 5 hours. The mixture was cooled, diluted with EtOAc, filtered through Celite, and concentrated. Silica gel chromatography using 0-100% EtOAc/Hex afforded 1.5 g (69%) of the desired product as a light brown foam. [M+Na] 525.

### Step 2: tert-butyl (R)-3-(5-chloro-7-(2-((2,5-dioxopyrrolidin-1-yl)methyl)thieno[3,2-b]pyridin-7-yl)-2,3-dihydrobenzofuran-2-yl)-6,7-dihydroisoxazolo[4,3-c]pyridine-5(4H)-carboxylate

tert-butyl 3-[5-chloro-7-(4,4,5,5-tetramethyl-1,3,2-dioxaborolan-2-yl)-2,3-dihydrobenzofuran-2-yl]-6,7-dihydro-4H-isoxazolo[4,3-c]pyridine-5-carboxylate (0.39 g, 0.78 mmol), 1-[(7-chlorothieno[3,2-b]pyridin-2-yl)methyl]pyrrolidine-2,5-dione (0.11 g, 0.39 mmol), Pd(OAc)₂ (8.7 mg, 0.039 mmol), X-phos (19 mg, 0.039 mmol), and K₃PO₄ (0.25 g, 1.16 mmol) were combined in 5:1 THF:H₂O (1.2 mL) and heated to 60° C. for 5 hours. The mixture was cooled and partitioned between EtOAc and water. The phases were separated and the organic phase was dried and concentrated. Silica gel chromatography (1-4% MeOH in CH₂Cl₂) furnished 49 mg (20%) of the desired product. [M+H] 621.0.

### Example 1

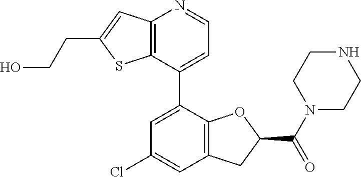 [SMILES: O=C([C@H]1Cc2cc(Cl)cc(-c3ccnc4cc(CCO)sc34)c2O1)N1CCNCC1]

### (R)-(5-chloro-7-(2-(2-hydroxyethyl)thieno[3,2-b]pyridin-7-yl)-2,3-dihydrobenzofuran-2-yl)(piperazin-1-yl)methanone

The example was prepared using precursor II, general procedure B with 2-(7-chlorothieno[3,2-b]pyridin-2-yl)ethan-1-ol and purified by reverse phase HPLC using water (0.1% TFA) and acetonitrile (0.1% TFA) as the eluent followed by using general procedures E and F successively. ¹H NMR (400 MHz, CD₃OD, free base): δ ppm 8.74 (d, J=4.8 Hz, 1H), 7.64 (d, J=4.8 Hz, 1H), 7.63 (d, J=2.0 Hz, 1H), 7.55-7.52 (m, 2H), 5.87 (dd, J=10.0, 6.8 Hz, 1H), 4.77 (s, 3H), 4.05 (t, J=6.0 Hz, 2H), 3.92-3.32 (m, 3H), 3.34 (t, J=6.0 Hz, 2H), 2.98-2.68 (4H). [M+H] 444.0.

### Example 2

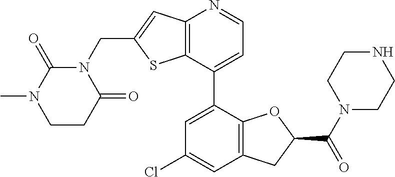 [SMILES: CN1CCC(=O)N(Cc2cc3nccc(-c4cc(Cl)cc5c4O[C@@H](C(=O)N4CCNCC4)C5)c3s2)C1=O]

(R)-3-((7-(5-chloro-2-(piperazine-1-carbonyl)-2,3-dihydrobenzofuran-7-yl)thieno[3,2-b]pyridin-2-yl)methyl)-1-methyldihydropyrimidine-2,4(1H,3H)-dione. The title compound was synthesized using precursor II, general procedure B, E and F using 3-((7-chlorothieno[3,2-b]pyridin-2-yl)methyl)-1-methyldihydropyrimidine-2,4(1H,3H)-dione to give the desired product. [M+H] 540.0.

### Example 3

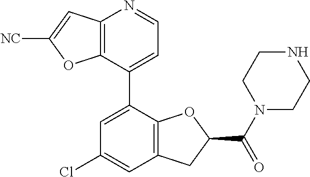 [SMILES: N#Cc1cc2nccc(-c3cc(Cl)cc4c3O[C@@H](C(=O)N3CCNCC3)C4)c2o1]

### (R)-7-(5-chloro-2-(piperazine-1-carbonyl)-2,3-dihydrobenzofuran-7-yl)furo[3,2-b]pyridine-2-carbonitrile

The example was prepared using precursor II, general procedure B with 7-chlorofuro[3,2-b]pyridine-2-carbonitrile and purified by reverse phase HPLC using water (0.1% TFA) and acetonitrile (0.1% TFA) as the eluent followed by using general procedures E and F successively. ¹H NMR (400 MHz, CD₃OD, free base): δ ppm 8.84 (d, J=5.2 Hz, 1H), 8.01 (s, 1H), 8.08 (d, J=5.2 Hz, 1H), 7.81 (d, J=2.4 Hz, 1H), 7.53 (d, J=2.4 Hz), 5.92 (dd, J=10.0, 7.2 Hz, 1H), 3.85-3.42 (m, 6H), 2.98-2.82 (m, 4H). [M+H] 409.0.

### Example 4

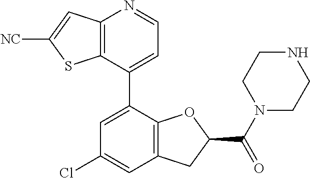 [SMILES: N#Cc1cc2nccc(-c3cc(Cl)cc4c3O[C@@H](C(=O)N3CCNCC3)C4)c2s1]

(R)-7-(5-chloro-2-(piperazine-1-carbonyl)-2,3-dihydrobenzofuran-7-yl)thieno[3,2-b]pyridine-2-carbonitrile. The example was prepared using precursor II, general procedure B with 7-chlorothieno[3,2-b]pyridine-2-carbonitrile and purified by reverse phase HPLC using water (0.1% TFA) and acetonitrile (0.1% TFA) as the eluent followed by using general procedures E and F successively. ¹H NMR (400 MHz, CD₃OD, free base): δ ppm 8.98 (d, J=5.2 Hz, 1H), 8.40 (s, 1H), 7.86 (d, J=5.2 Hz, 1H), 7.58 (d, J=2.4 Hz, 1H), 7.55 (d, J=2.4 Hz), 5.90 (dd, J=10.0, 6.8 Hz, 1H), 3.86-3.42 (m, 6H), 2.96-2.62 (m, 4H). [M+H] 425.0.

### Example 5

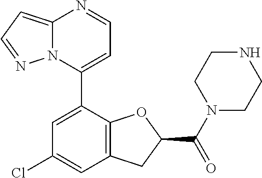 [SMILES: O=C([C@H]1Cc2cc(Cl)cc(-c3ccnc4ccnn34)c2O1)N1CCNCC1]

### (R)-(5-chloro-7-(pyrazolo[1,5-a]pyrimidin-7-yl)-2,3-dihydrobenzofuran-2-yl)(piperazin-1-yl)methanone

The example was prepared using precursor II, general procedure B with 7-chloropyrazolo[1,5-a]pyrimidine and purified by reverse phase HPLC using water (0.1% TFA) and acetonitrile (0.1% TFA) as the eluent followed by using general procedures E and F successively. ¹H NMR (400 MHz, CD₃OD, free base): δ ppm 8.68 (d, J=4.0 Hz, 1H), 8.32 (d, J=2.4 Hz, 1H), 8.00 (t, J=1.0 Hz, 1H), 7.59 (t, J=1.0 Hz, 1H), 7.44 (d, J=4.0 Hz, 1H), 6.92 (d, J=2.4 Hz, 1H), 5.89 (dd, J=10.0, 6.5 Hz, 1H), 3.85-3.52 (m, 6H), 2.92-2.67 (m, 4H). [M+H] 384.0.

### Example 6

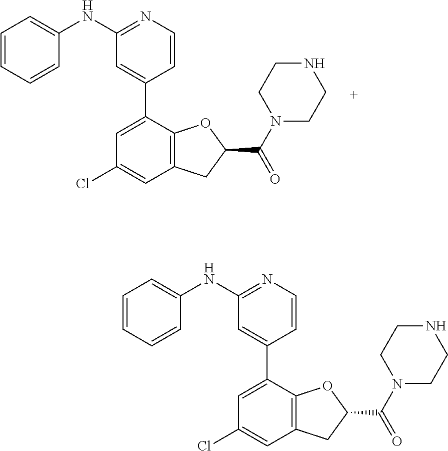 [SMILES: O=C([C@H]1Cc2cc(Cl)cc(-c3ccnc(Nc4ccccc4)c3)c2O1)N1CCNCC1 ; O=C([C@@H]1Cc2cc(Cl)cc(-c3ccnc(Nc4ccccc4)c3)c2O1)N1CCNCC1]

(R)-(5-chloro-7-(2-(phenylamino)pyridin-4-yl)-2,3-dihydrobenzofuran-2-yl)(piperazin-1-yl)methanone and (S)-(5-chloro-7-(2-(phenylamino)pyridin-4-yl)-2,3-dihydrobenzofuran-2-yl)(piperazin-1-yl)methanone. The example was prepared using precursor I, general procedure C with 4-bromo-N-phenylpyridin-2-amine and purified by silica gel chromatography using EtOAc and hexanes (0 to 100% gradient) as eluent followed by using general procedure D. ¹H NMR (400 MHz, CD₃OD, hydrochloric acid salt): δ ppm 7.88 (d, J=6.8 Hz, 1H), 7.70 (s, 1H), 7.57 (m, 3H), 7.44-7.40 (m, 4H), 5.88 (dd, J=9.6, 6.0 Hz, 1H), 3.96-3.28 (m, 10H). [M+H] 435.0.

### Example 7

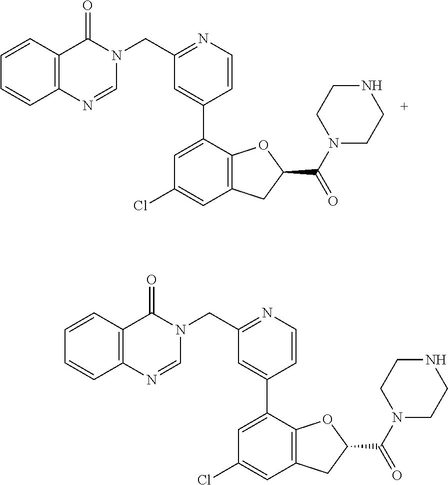 [SMILES: O=C([C@H]1Cc2cc(Cl)cc(-c3ccnc(Cn4cnc5ccccc5c4=O)c3)c2O1)N1CCNCC1 ; O=C([C@@H]1Cc2cc(Cl)cc(-c3ccnc(Cn4cnc5ccccc5c4=O)c3)c2O1)N1CCNCC1]

(R)-3-((4-(5-chloro-2-(piperazine-1-carbonyl)-2,3-dihydrobenzofuran-7-yl)pyridin-2-yl)methyl)quinazolin-4(3H)-one and (S)-3-((4-(5-chloro-2-(piperazine-1-carbonyl)-2,3-dihydrobenzofuran-7-yl)pyridin-2-yl)methyl)quinazolin-4(3H)-one. The example was prepared using precursor I, general procedure C with 3-((4-bromopyridin-2-yl)methyl)quinazolin-4(3H)-one and purified by silica gel chromatography using EtOAc and hexanes (0 to 100% gradient) as eluent followed by using general procedure D. ¹H NMR (400 MHz, CD₃OD, hydrochloric acid salt): δ ppm 9.17 (s, 1H), 8.82 (d, J=6.0 Hz, 1H), 8.60 (s, 1H), 8.40 (d, J=4.8 Hz, 1H), 8.29 (d, J=6.8 Hz, 1H), 8.00 (t, J=6.8 Hz, 1H), 7.84 (bs, 1H), 7.71 (t, J=6.8H, 1H), 7.45 (s, 1H), 5.89 (t, J=5.6 Hz, 1H), 5.70 (s, 2H), 3.91-3.27 (m, 10H). [M+H] 502.0.

### Example 8

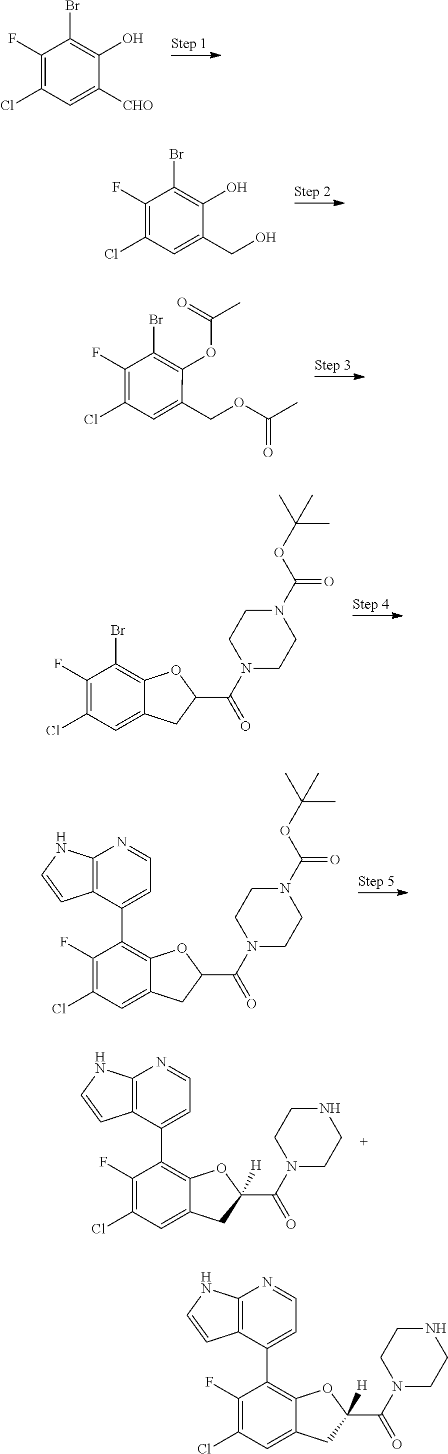

### Step 1: 2-bromo-4-chloro-3-fluoro-6-(hydroxymethyl)phenol

To a solution of 3-bromo-5-chloro-4-fluoro-2-hydroxybenzaldehyde (2 g, 7.9 mmol) in THF (25 mL) was added NaBH₄ (1.6 g, 34 mmol). The reaction was stirred at rt for 2 h. The mixture was quenched with H₂O and 1N HCl, extracted with EtOAc. The organic layer was washed with NaHCO₃, brine, dried, concentrated and purified with flash chromatography (20-40% EtOAc in hexanes) to give the title compound (2.0 g, 99%) without further purification.

### Step 2: 2-acetoxy-3-bromo-5-chloro-4-fluorobenzyl Acetate

To a solution of 2-bromo-4-chloro-3-fluoro-6-(hydroxymethyl)phenol (1.0 g, 3.9 mmol) in DCM (15 mL) was added pyridine (1.85 g, 23.4 mmol), AC₂O (1.1 mL, 11.7 mmol), followed by DMAP (6 mg). The reaction was stirred at rt for 2 hr. The mixture was extracted with EtOAc, 1N HCl, H₂O. The organic layer was washed with NaHCO₃, brine, dried and concentrated to give the title compound (1.16 g, 88%) without further purification.

### Step 3: tert-butyl 4-(7-bromo-5-chloro-6-fluoro-2,3-dihydrobenzofuran-2-carbonyl)piperazine-1-carboxylate

To a mixture of 2-acetoxy-3-bromo-5-chloro-4-fluorobenzyl acetate (328 mg, 0.97 mmol), piperazine pyridinium bromide salt (373 mg, 0.97 mmol) in MeCN (8 mL) was added DBU (0.3 mL, 1.93 mmol) in a sealed tube. The reaction was heated to reflux for 6 h. The mixture was concentrated and purified by flash column chromatography (5-25% EtOAc/hexanes) to give the title compound (110 mg, 24%).

### Step 4: tert-butyl 4-(5-chloro-6-fluoro-7-(1H-pyrrolo[2,3-b]pyridin-4-yl)-2,3-dihydrobenzofuran-2-carbonyl)piperazine-1-carboxylate

A mixture of tert-butyl 4-(7-bromo-5-chloro-6-fluoro-2,3-dihydrobenzofuran-2-carbonyl)piperazine-1-carboxylate (60 mg, 0.13 mmol) and 4-(4,4,5,5-tetramethyl-1,3,2-dioxaborolan-2-yl)-1H-pyrrolo[2,3-b]pyridine (50 mg, 1.3 eq), K₂CO₃ (54 mg, 0.4 mmol) and Pd(tetrakis) (15 mg, 0.013 mmol) in dioxane and H₂O (4:1, 3 mL) under N₂ was heated to 100° C. for 3 h while stirring. The reaction was quenched with H₂O, extracted with EtOAc and washed with H₂O, NaHCO₃ solution and brine. The crude was concentrated and purified by flash column chromatography (50-90% EtOAc in hexanes) to give the title compound (45 mg, 69%).

### Step 5: ((2R)-5-chloro-6-fluoro-7-(1H-pyrrolo[2,3-b]pyridin-4-yl)-2,3-dihydrobenzofuran-2-yl)(piperazin-1-yl)methanone and ((2S)-5-chloro-6-fluoro-7-(1H-pyrrolo[2,3-b]pyridin-4-yl)-2,3-dihydrobenzofuran-2-yl)(piperazin-1-yl)methanone

To a solution of tert-butyl 4-(5-chloro-6-fluoro-7-(1H-pyrrolo[2,3-b]pyridin-4-yl)-2,3-dihydrobenzofuran-2-carbonyl)piperazine-1-carboxylate (30 mg, 0.1 mmol) in DCM (2 mL) was added TFA (1 mL) at rt The reaction was stirred for 1 h and concentrated in vacuo. The mixture was treated with HCl (4M in dioxane, 0.3 mL) followed by 10 mL EtOAc, After the solvent was carefully removed, the crude product was concentrated and dried on vacuum to give the title compounds (36 mg, 91%). ¹H NMR (400 MHz, CD₃OD, hydrochloric acid salt): δ ppm 9.59 (bs, 1H), 9.51 (bs, 1H), 7.78-7.60 (m, 3H), 6.64 (dd, J=9.5, 2.5 Hz, 1H), 5.46 (dd, J=9.6, 2.4 Hz, 1H), 3.98-3.18 (m, 10H). [M+H] 417.1.

### Example 9

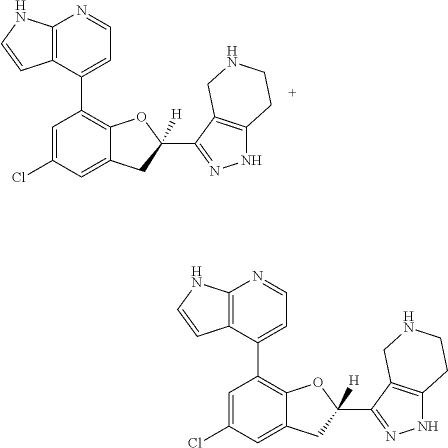 [SMILES: Clc1cc2c(c(-c3ccnc4[nH]ccc34)c1)O[C@H](c1n[nH]c3c1CNCC3)C2 ; Clc1cc2c(c(-c3ccnc4[nH]ccc34)c1)O[C@@H](c1n[nH]c3c1CNCC3)C2]

### (R)-3-(5-chloro-7-(1H-pyrrolo[2,3-b]pyridin-4-yl)-2,3-dihydrobenzofuran-2-yl)-4,5,6,7-tetrahydro-1H-pyrazolo[4,3-c]pyridine and (S)-3-(5-chloro-7-(1H-pyrrolo[2,3-b]pyridin-4-yl)-2,3-dihydrobenzofuran-2-yl)-4,5,6,7-tetrahydro-1H-pyrazolo[4,3-c]pyridine

The title compound was synthesized using precursor IV, general procedure C and D using 4-(4,4,5,5-tetramethyl-1,3,2-dioxaborolan-2-yl)-1H-pyrrolo[2,3-b]pyridine to give the desired compound as the HCl salt. ¹H NMR (400 MHz, DMSO-d₆, hydrochloric acid salt): δ ppm 12.45 (bs, 1H), 9.42 (bs, 2H), 8.51 (d, J=5.4 Hz, 1H), 7.76 (s, 1H), 7.63 (s, 1H), 7.60 (s, 1H), 7.53 (d, J=5.6 Hz, 1H), 6.69 (d, J=1.0 Hz, 1H), 6.21 (t, J=8.0 Hz, 1H), 4.05-2.98 (m, 8H). [M+H] 392.1.

### Example 10

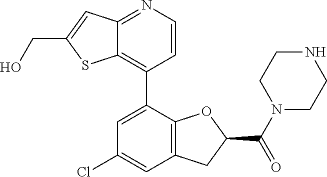 [SMILES: O=C([C@H]1Cc2cc(Cl)cc(-c3ccnc4cc(CO)sc34)c2O1)N1CCNCC1]

### (R)-(5-chloro-7-(2-(hydroxymethyl)thieno[3,2-b]pyridin-7-yl)-2,3-dihydrobenzofuran-2-yl)(piperazin-1-yl)methanone

The example was prepared using precursor II, general procedure B with (7-chlorothieno[3,2-b]pyridin-2-yl)methanol and purified by reverse phase HPLC using water (0.1% TFA) and acetonitrile (0.1% TFA) as the eluent followed by using general procedures E and F successively. ¹H NMR (400 MHz, CD₃OD, free base): δ ppm 8.75 (d, J=4.4 Hz, 1H), 7.63 (d, J=4.8 Hz, 1H), 7.61 (d, J=2.0 Hz, 1H), 7.57 (s, 1H), 7.54 (d, J=2.0 Hz, 1H), 5.88 (dd, J=9.6, 6.8 Hz, 1H), 5.06 (s, 2H), 3.92-3.40 (m, 6H), 2.92-2.70 (m, 4H). [M+H] 430.0.

### Example 11

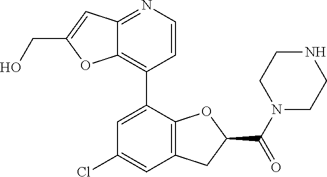 [SMILES: O=C([C@H]1Cc2cc(Cl)cc(-c3ccnc4cc(CO)oc34)c2O1)N1CCNCC1]

### (R)-(5-chloro-7-(2-(hydroxymethyl)furo[3,2-b]pyridin-7-yl)-2,3-dihydrobenzofuran-2-yl)(piperazin-1-yl)methanone

The example was prepared using precursor II, general procedure B with (7-chlorofuro[3,2-b]pyridin-2-yl)methanol and purified by reverse phase HPLC using water (0.1% TFA) and acetonitrile (0.1% TFA) as the eluent followed by using general procedures E and F successively. ¹H NMR (400 MHz, CD₃OD, free base): δ ppm 8.46 (bs, 1H), 7.72 (s, 1H), 7.36 (s, 1H), 6.93 (s, 1H), 5.77 (dd, J=9.6, 6.8 Hz, 1H), 4.77 (s, 2H), 3.71-3.43 (m, 6H), 2.80-2.70 (m, 4H). [M+H] 414.0.

### Example 12

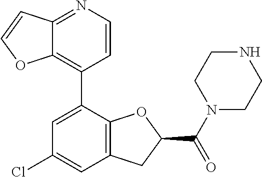 [SMILES: O=C([C@H]1Cc2cc(Cl)cc(-c3ccnc4ccoc34)c2O1)N1CCNCC1]

### (R)-(5-chloro-7-(furo[3,2-b]pyridin-7-yl)-2,3-dihydrobenzofuran-2-yl)(piperazin-1-yl)methanone

The example was prepared using precursor II, general procedure B with 7-chlorofuro[3,2-b]pyridine and purified by reverse phase HPLC using water (0.1% TFA) and acetonitrile (0.1% TFA) as the eluent followed by using general procedure D. ¹H NMR (400 MHz, CD₃OD, hydrochloric acid salt): δ ppm 8.67 (s, 1H), 8.51 (bs, 1H), 7.98 (d, J=2.0 Hz, 1H), 7.67-7.54 (m, 2H), 7.45 (bs, 1H), 5.96 (dd, J=10.0, 6.8 Hz, 1H), 4.87 (s, 2H), 3.71-3.46 (m, 10H). [M+H] 383.9.

### Example 13

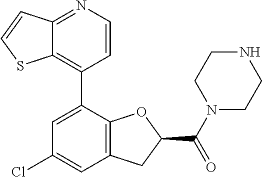 [SMILES: O=C([C@H]1Cc2cc(Cl)cc(-c3ccnc4ccsc34)c2O1)N1CCNCC1]

### (R)-(5-chloro-7-(thieno[3,2-b]pyridin-7-yl)-2,3-dihydrobenzofuran-2-yl)(piperazin-1-yl)methanone

The example was prepared using precursor II, general procedure B with 7-chlorothieno[3,2-b]pyridine and purified by reverse phase HPLC using water (0.1% TFA) and acetonitrile (0.1% TFA) as the eluent followed by using general procedure D. ¹H NMR (400 MHz, CD₃OD, hydrochloric acid salt): δ ppm 8.64 (s, 1H), 8.21 (bs, 1H), 7.86 (bs, 1H), 7.22 (bs, 1H), 7.67 (bs, 1H), 7.53 (s, 1H), 5.89 (m, 1H), 3.86-3.09 (m, 10H). [M+H] 400.0.

### Example 14

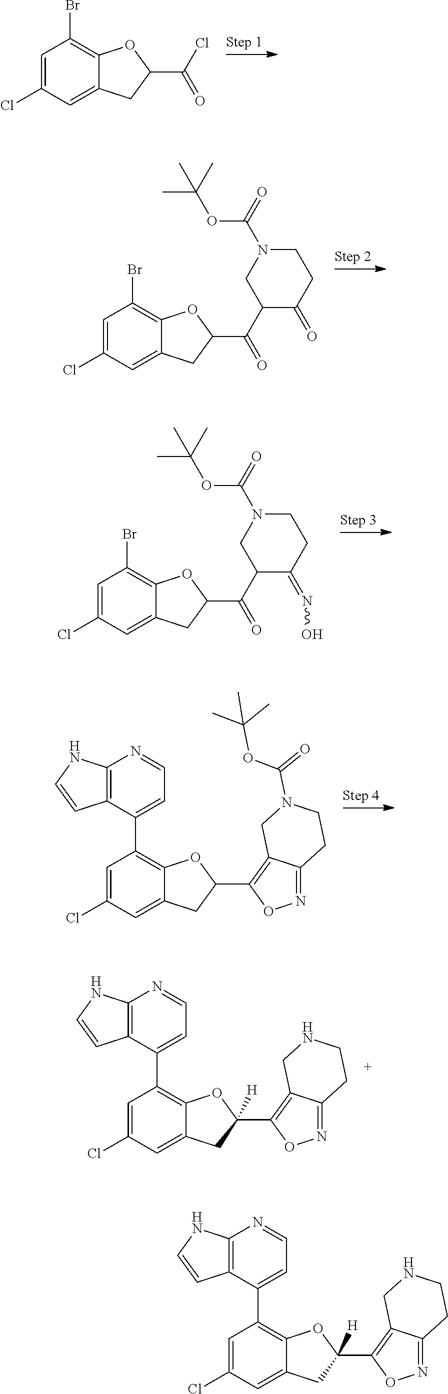 [SMILES: O=C(Cl)C1Cc2cc(Cl)cc(Br)c2O1 ; CC(C)(C)OC(=O)N1CCC(=O)C(C(=O)C2Cc3cc(Cl)cc(Br)c3O2)C1 ; CC(C)(C)OC(=O)N1CCC(=NO)C(C(=O)C2Cc3cc(Cl)cc(Br)c3O2)C1 ; CC(C)(C)OC(=O)N1CCc2noc(C3Cc4cc(Cl)cc(-c5ccnc6[nH]ccc56)c4O3)c2C1 ; Clc1cc2c(c(-c3ccnc4[nH]ccc34)c1)O[C@H](c1onc3c1CNCC3)C2 ; Clc1cc2c(c(-c3ccnc4[nH]ccc34)c1)O[C@@H](c1onc3c1CNCC3)C2]

### Step 1: tert-butyl 3-(7-bromo-5-chloro-2,3-dihydrobenzofuran-2-carbonyl)-4-oxopiperidine-1-carboxylate

To a cooled solution (−78° C.) of tert-butyl 4-oxopiperidine-1-carboxylate (573 mg, 2.88 mmol) in anhydrous THF (8 mL) was added a solution of LDA (2.88 mL, 1M in hexanes, 2.88 mmol). After stirring at −78° C. for 30 min, the reaction was added 7-bromo-5-chloro-2,3-dihydrobenzofuran-2-carbonyl chloride (500 mg, in 2 mL THF, 1.7 mmol), stirred at −78° C. for 30 min and then warmed to 0° C. The reaction was quenched with aqueous NH₄Cl at 0° C. and the aqueous layer was extracted with EtOAc. The combined organic layers were concentrated. The crude product was purified by flash column chromatography using 5-25% EtOAc/hexanes to give the title compound (249 mg, 32%).

### Step 2: tert-butyl 3-(7-bromo-5-chloro-2,3-dihydrobenzofuran-2-carbonyl)-4-(hydroxyimino)piperidine-1-carboxylate

tert-butyl 3-(7-bromo-5-chloro-2,3-dihydrobenzofuran-2-carbonyl)-4-oxopiperidine-1-carboxylate (125 mg, 0.272 mmol) was dissolved in EtOH (3 mL) and was added NH₂OH—HCl salt (32 mg, 0.46 mmol) and NaOAc (50 mg, 0.6 mmol). The reaction was heated at 78° C. and stirred for 3 h. The reaction mixture was cooled and concentrated. The reaction mixture was diluted with water and extracted with EtOAc. The organic layers were concentrated and the crude product was purified by flash column chromatography (20-40% EtOAc/hexanes) to give a mixture of two diastereomers (90 mg, 73%).

### Step 3: tert-butyl 3-(5-chloro-7-(1H-pyrrolo[2,3-b]pyridin-4-yl)-2,3-dihydrobenzofuran-2-yl)-6,7-dihydroisoxazolo[4,3-c]pyridine-5 (4H)-carboxylate

A mixture of compound tert-butyl 3-(7-bromo-5-chloro-2,3-dihydrobenzofuran-2-carbonyl)-4-(hydroxyimino)piperidine-1-carboxylate (30 mg, 0.066 mmol), 4-(4,4,5,5-tetramethyl-1,3,2-dioxaborolan-2-yl)-1H-pyrrolo[2,3-b]pyridine (24 mg, 0.099 mmol), tetrakis(triphenylphosphine)palladium(0) (7.6 mg, 0.0066 mmol), and K₂CO₃ (27 mg, 0.2 mmol) in dioxane (1.5 mL) and water (0.5 mL) was heated at 100° C. and stirred for 3 h. The reaction mixture was cooled to room temperature, diluted with water and extracted with EtOAc. The organic layers were concentrated. The crude product was purified by flash column chromatography (25-65% EtOAc/hexanes) to give the title compound (11.7 mg, 36%).

### Step 4: (R)-3-(5-chloro-7-(1H-pyrrolo[2,3-b]pyridin-4-yl)-2,3-dihydrobenzofuran-2-yl)-4,5,6,7-tetrahydroisoxazolo[4,3-c]pyridine and (S)-3-(5-chloro-7-(1H-pyrrolo[2,3-b]pyridin-4-yl)-2,3-dihydrobenzofuran-2-yl)-4,5,6,7-tetrahydroisoxazolo[4,3-c]pyridine

tert-butyl 3-(5-chloro-7-(1H-pyrrolo[2,3-b]pyridin-4-yl)-2,3-dihydrobenzofuran-2-yl)-6,7-dihydroisoxazolo[4,3-c]pyridine-5(4H)-carboxylate (11.7 mg, 0.023 mmol) was dissolved in DCM (1 mL) and TFA (1 mL) was then added. The reaction was stirred at rt for 1 h, and concentrated. The reaction mixture was treated with HCl (4M, in dioxane, 0.3 mL) and ether (2 mL) was added. The product was precipitated out as the HCl salt and the ether layer was removed with pipette. The product was dried to give the title compounds (9.7 mg, 98%). ¹H NMR (400 MHz, DMSO-d₆, hydrochloric acid salt): δ ppm 12.25 (bs, 1H), 9.65 (bs, 2H), 8.47 (d, J=5.2 Hz, 1H), 7.73 (s, 1H), 7.66 (d, J=2.1 Hz, 1H), 7.62 (d, J=2.6 Hz, 1H), 7.44 (d, J=5.1 Hz, 1H), 6.62 (d, J=1.2 Hz, 1H), 6.21 (t, J=9.6 Hz, 1H), 4.21-3.14 (m, 8H). [M+H] 393.1.

### Example 15

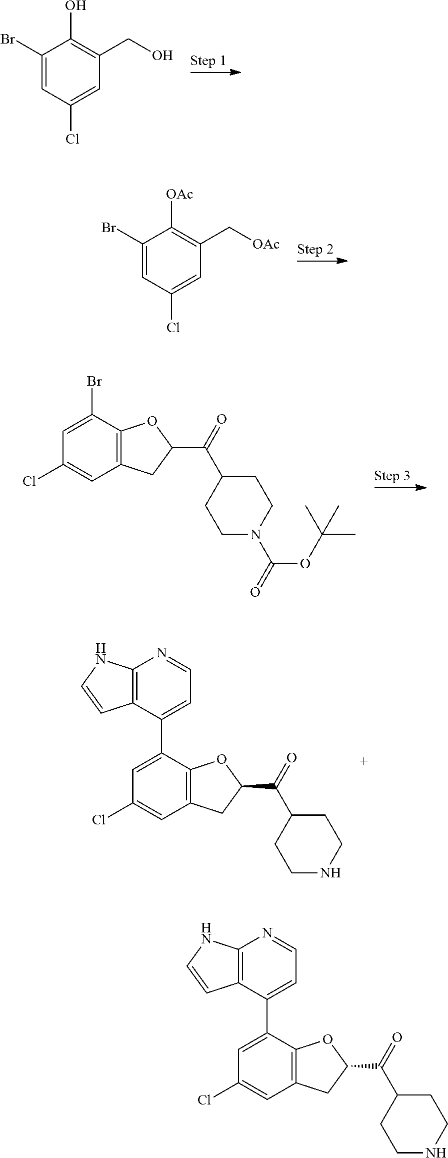 [SMILES: OCc1cc(Cl)cc(Br)c1O ; CC(=O)OCc1cc(Cl)cc(Br)c1OC(C)=O ; CC(C)(C)OC(=O)N1CCC(C(=O)C2Cc3cc(Cl)cc(Br)c3O2)CC1 ; O=C(C1CCNCC1)[C@H]1Cc2cc(Cl)cc(-c3ccnc4[nH]ccc34)c2O1 ; O=C(C1CCNCC1)[C@@H]1Cc2cc(Cl)cc(-c3ccnc4[nH]ccc34)c2O1]

### Step 1: 2-acetoxy-3-bromo-5-chlorobenzyl Acetate

2-bromo-4-chloro-6-(hydroxymethyl)phenol (3.55 g, 15 mmol) was dissolved in DCM (60 mL) and the solution was treated with acetyl chloride (2.66 mL, 37.5 mmol) and followed by TEA (4.2 g, 41.3 mmol). The reaction was stirred at rt overnight, and then concentrated. The reaction mixture was diluted with NaHCO₃ (100 mL) and extracted with EtOAc. The organic layer was concentrated and purified by flash column chromatography (5-20% EtOAc/hexanes) to give the title compound (826 mg, 17%).

### Step 2: tert-butyl 4-(7-bromo-5-chloro-2,3-dihydrobenzofuran-2-carbonyl)piperidine-1-carboxylate

DBU (0.77 mL, 5.15 mmol) was added to a mixture of 2-acetoxy-3-bromo-5-chlorobenzyl acetate (826 mg, 2.57 mmol) and 1-(2-(1-(tert-butoxycarbonyl)piperidin-4-yl)-2-oxoethyl)pyridin-1-ium bromide (990 mg, 2.57 mmol) in dry CH₃CN (15 mL). The reaction was refluxed for 6 h, and cooled and then concentrated. The reaction mixture was purified by flash column chromatography (5-25% EtOAc/hexanes) to give the title compound (721 mg, 63%).

### Step 3: (R)-(5-chloro-7-(1H-pyrrolo[2,3-b]pyridin-4-yl)-2,3-dihydrobenzofuran-2-yl)(piperidin-4-yl)methanone and (S)-(5-chloro-7-(1H-pyrrolo[2,3-b]pyridin-4-yl)-2,3-dihydrobenzofuran-2-yl)(piperidin-4-yl)methanone

The title compounds were prepared using general procedure C with 4-(4,4,5,5-tetramethyl-1,3,2-dioxaborolan-2-yl)-1H-pyrrolo[2,3-b]pyridine followed by using general procedures E and F successively. ¹H NMR (400 MHz, DMSO-d₆, hydrochloric acid salt): δ ppm 12.20 (bs, 1H), 9.00 (bs, 1H), 8.78 (bs, 1H), 8.35 (d, J=5.2 Hz, 1H), 7.62 (d, J=3.2 Hz, 1H), 7.43 (d, J=2.0 Hz, 1H), 7.37 (d, J=5.2 Hz, 1H), 6.64 (d, J=1.6 Hz, 1H), 5.62 (dd, J=10.8, 6.8 Hz, 1H), 3.61-1.93 (m, 11H). [M+H] 382.2.

### Example 16

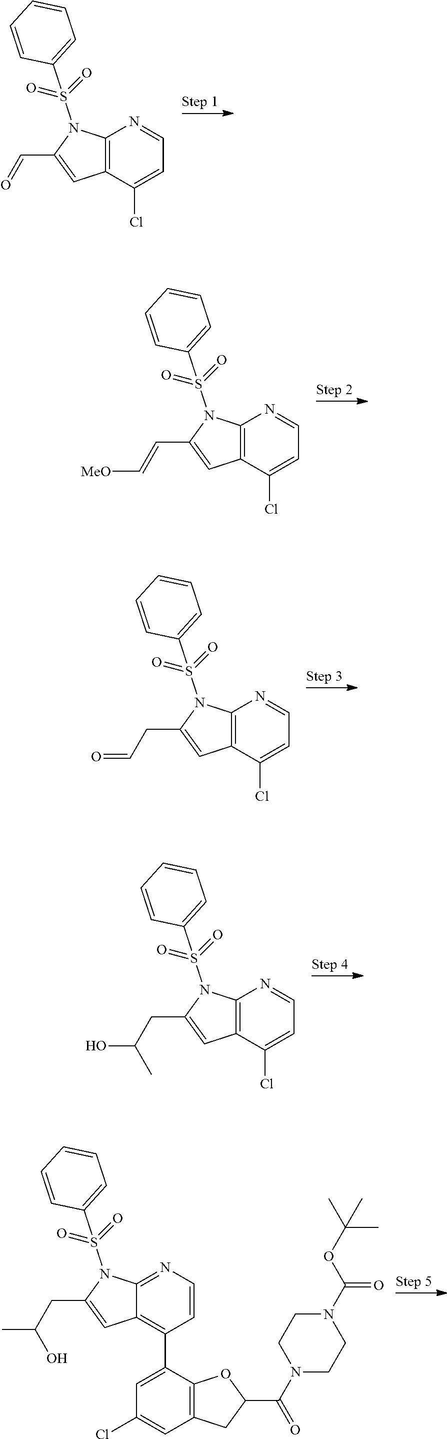 [SMILES: O=Cc1cc2c(Cl)ccnc2n1S(=O)(=O)c1ccccc1 ; CO/C=C/c1cc2c(Cl)ccnc2n1S(=O)(=O)c1ccccc1 ; O=CCc1cc2c(Cl)ccnc2n1S(=O)(=O)c1ccccc1 ; CC(O)Cc1cc2c(Cl)ccnc2n1S(=O)(=O)c1ccccc1 ; CC(O)Cc1cc2c(-c3cc(Cl)cc4c3OC(C(=O)N3CCN(C(=O)OC(C)(C)C)CC3)C4)ccnc2n1S(=O)(=O)c1ccccc1]

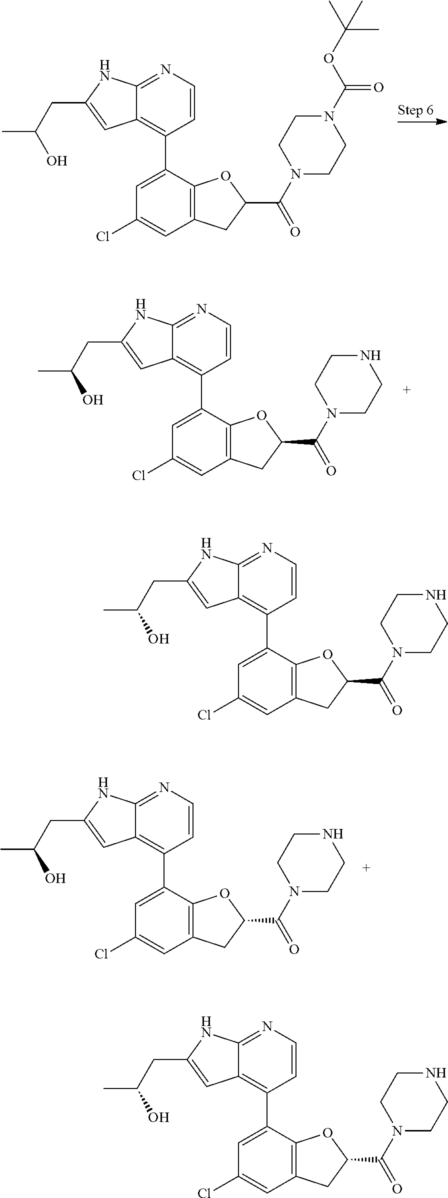 [SMILES: CC(O)Cc1cc2c(-c3cc(Cl)cc4c3OC(C(=O)N3CCN(C(=O)OC(C)(C)C)CC3)C4)ccnc2[nH]1 ; C[C@H](O)Cc1cc2c(-c3cc(Cl)cc4c3O[C@@H](C(=O)N3CCNCC3)C4)ccnc2[nH]1 ; C[C@@H](O)Cc1cc2c(-c3cc(Cl)cc4c3O[C@@H](C(=O)N3CCNCC3)C4)ccnc2[nH]1 ; C[C@H](O)Cc1cc2c(-c3cc(Cl)cc4c3O[C@H](C(=O)N3CCNCC3)C4)ccnc2[nH]1 ; C[C@@H](O)Cc1cc2c(-c3cc(Cl)cc4c3O[C@H](C(=O)N3CCNCC3)C4)ccnc2[nH]1]

### Step 1: (E)-4-chloro-2-(2-methoxyvinyl)-1-(phenylsulfonyl)-1H-pyrrolo[2,3-b]pyridine

To a cooled solution (−78° C.) of 4-chloro-1-(phenylsulfonyl)-1H-pyrrolo[2,3-b]pyridine-2-carbaldehyde (3.2 g, 9.375 mmol) in anhydrous THF (30 mL) was added a solution of LiHMDS (9.4 mL, 1M in THF, 9.4 mmol). The reaction was warmed to 0° C. and (methoxymethyl)triphenyphosphonium bromide (2 g, 6.25 mmol) was added. The reaction was stirred at 0° C. for 1 h and quenched with aqueous NH₄Cl. The aqueous layer was extracted with EtOAc. The combined organic layers were concentrated. The crude product was purified with column chromatography using 10-30% EtOAc/hexanes to give the title compound (1.8 g, 83%).

### Step 2: 2-(4-chloro-1-(phenylsulfonyl)-1H-pyrrolo[2,3-b]pyridin-2-yl)acetaldehyde

(E)-4-chloro-2-(2-methoxyvinyl)-1-(phenylsulfonyl)-1H-pyrrolo[2,3-b]pyridine (1.8 g, 5.17 mmol) was dissolved in dioxane (50 mL) and was treated with HCl (6 M in dioxane, 6 mL). The reaction was stirred at 60° C. for 1 hr. The reaction mixture was neutralized with aqueous NaHCO₃ and extracted with EtOAc. The organic layers were washed with brine, and dried over MgSO₄. The product was filtered and concentrated to dryness to give the title compound without further purification.

### Step 3: 1-(4-chloro-1-(phenylsulfonyl)-1H-pyrrolo[2,3-b]pyridin-2-yl)propan-2-ol

To a cooled solution (0° C.) of 2-(4-chloro-1-(phenylsulfonyl)-1H-pyrrolo[2,3-b]pyridin-2-yl)acetaldehyde (1.8 g, 5.38 mmol) in anhydrous THF (30 mL) was added a solution of MeMgCl (3 M in THF, 10.8 mmol, 3.8 mL). The reaction was stirred at 0° C. for 2 h, and then quenched with saturated NH₄Cl. The aqueous layer was extracted with EtOAc. The combined organic layers were washed with brine and concentrated. The crude product was purified by column chromatography using 10-50% EtOAc/hexanes to give the title compound (950 mg, 50%).

### Step 4: tert-butyl 4-(5-chloro-7-(2-(2-hydroxypropyl)-1-(phenylsulfonyl)-1H-pyrrolo[2,3-b]pyridin-4-yl)-2,3-dihydrobenzofuran-2-carbonyl)piperazine-1-carboxylate

The title compound was prepared using general procedure B with 1-(4-chloro-1-(phenylsulfonyl)-1H-pyrrolo[2,3-b]pyridin-2-yl)propan-2-ol and purified by column chromatography using 0-100% EtOAc/hexanes.

### Step 5: tert-butyl 4-(5-chloro-7-(2-(2-hydroxypropyl)-1H-pyrrolo[2,3-b]pyridin-4-yl)-2,3-dihydrobenzofuran-2-carbonyl)piperazine-1-carboxylate

tert-butyl 4-(5-chloro-7-(2-(2-hydroxypropyl)-1-(phenylsulfonyl)-1H-pyrrolo[2,3-b]pyridin-4-yl)-2,3-dihydrobenzofuran-2-carbonyl)piperazine-1-carboxylate (400 mg, 0.587 mmol) was dissolved in THF/MeOH (1:1) (4 mL) and Cs₂CO3 (763 mg, 2.35 mmol) was added. The reaction was stirred at 50° C. for 1 h. The reaction mixture was concentrated and extracted with EtOAc. The organic layers were concentrated and purified by chromatography (3-7% MeOH/EtOAc) to give the title compound (149 mg, 47%).

### Step 6: ((R)-5-chloro-7-(2-((S)-2-hydroxypropyl)-1H-pyrrolo[2,3-b]pyridin-4-yl)-2,3-dihydrobenzofuran-2-yl)(piperazin-1-yl)methanone and ((R)-5-chloro-7-(2-((R)-2-hydroxypropyl)-1H-pyrrolo[2,3-b]pyridin-4-yl)-2,3-dihydrobenzofuran-2-yl)(piperazin-1-yl)methanone and ((S)-5-chloro-7-(2-((S)-2-hydroxypropyl)-1H-pyrrolo[2,3-b]pyridin-4-yl)-2,3-dihydrobenzofuran-2-yl)(piperazin-1-yl)methanone and ((S)-5-chloro-7-(2-((R)-2-hydroxypropyl)-1H-pyrrolo[2,3-b]pyridin-4-yl)-2,3-dihydrobenzofuran-2-yl)(piperazin-1-yl)methanone

The title compounds were prepared using general procedures E and F successively. ¹H NMR (400 MHz, CD₃OD, free base): δ ppm 8.12 (d, J=4.8 Hz, 1H), 7.40 (s, 1H), 7.30 (d, J=3.2 Hz, 1H), 7.30 (d, J=2.4 Hz, 1H), 7.23 (dd, J=5.2, 2.4 Hz, 1H), 6.34 (d, J=6.4 Hz, 1H), 5.70 (dd, J=10.0, 7.2 Hz, 1H), 4.11 (septet, J=6.4 Hz, 1H), 3.96-2.73 (m, 12H), 1.22 (d, J=6.4 Hz, 3H). [M+H] 441.1.

### Example 17

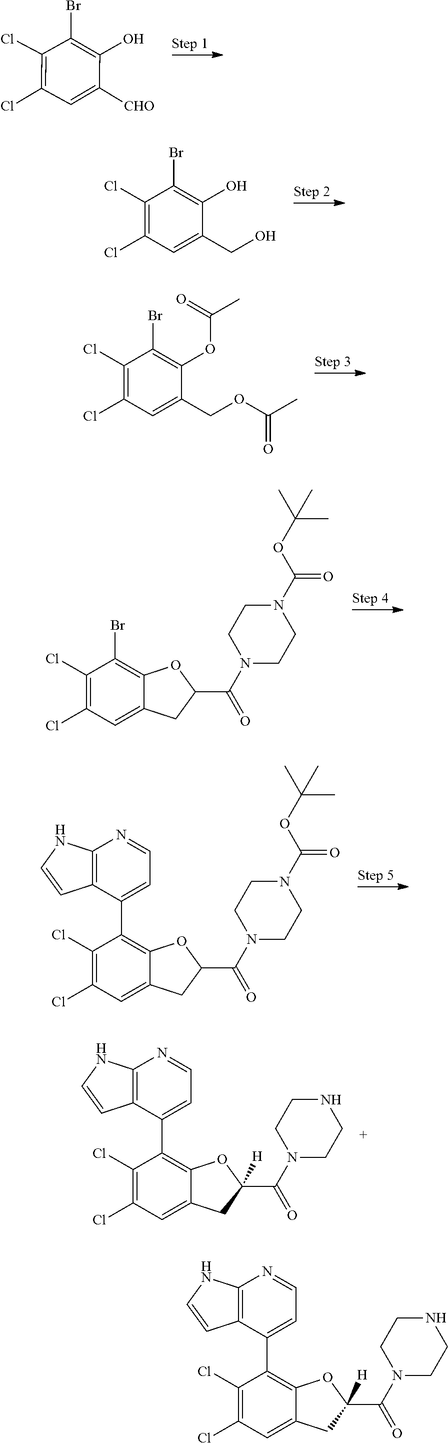

### Step 1: 2-bromo-3,4-dichloro-6-(hydroxymethyl)phenol

To a solution of 3-bromo-4,5-dichloro-2-hydroxybenzaldehyde (300 mg, 1 mmol) in THF (3 mL) was added NaBH₄ (150 mg, 3 eq). The reaction was stirred at rt for 2 hr. The mixture was quenched with H₂O and 1N HCl, extracted with EtOAc. The organic layer was washed with NaHCO₃, brine, dried, concentrated and purified with chromatography (20-40% EtOAc in Hexanes) to give the title compound (300 mg) without further purification.

### Step 2: 2-acetoxy-3-bromo-4,5-dichlorobenzyl Acetate

To a solution of 2-bromo-3,4-dichloro-6-(hydroxymethyl)phenol (60 mg, 0.22 mmol) in DCM (3 mL) was added pyridine (105 mg, 6 eq), AC₂O (63 uL, 3 eq), followed by DMAP (6 mg). The reaction was stirred at rt for 2 hr. The mixture was extracted with EtOAc, 1N HCl, H₂O. The organic layer was washed with NaHCO₃, brine, dried and concentrated to give the title compound (270 mg) without further purification.

### Step 3: tert-butyl 4-(7-bromo-5,6-dichloro-2,3-dihydrobenzofuran-2-carbonyl)piperazine-1-carboxylate

To a mixture of 2-acetoxy-3-bromo-4,5-dichlorobenzyl acetate (270 mg, 0.21 mmol), piperazine pyridinium bromide salt (320 mg, 1 eq) in MeCN (6 mL) was added DBU (250 uL, 2.1 eq) in a sealed tube. The reaction was heated to reflux for 6 hr. The mixture was concentrated and purified by chromatography to give the title compound (60 mg).

### Step 4: tert-butyl 4-(5,6-dichloro-7-(1H-pyrrolo[2,3-b]pyridin-4-yl)-2,3-dihydrobenzofuran-2-carbonyl)piperazine-1-carboxylate

A mixture of tert-butyl 4-(7-bromo-5,6-dichloro-2,3-dihydrobenzofuran-2-carbonyl)piperazine-1-carboxylate (60 mg, 0.1 mmol) and 4-(4,4,5,5-tetramethyl-1,3,2-dioxaborolan-2-yl)-1H-pyrrolo[2,3-b]pyridine (50 mg, 1.3 eq), K₂CO₃ (70 mg, 3 eq) and Pd(tetrakis) (20 mg, 0.1 eq) in dioxane and H₂O (4:1, 3 mL) under N₂ was heated to 100° C. for 3 h while stirring. The reaction was quenched with H₂O, extracted with EtOAc and washed with H₂O, NaHCO₃ solution and brine. The crude was concentrated and purified chromatography (50-90% EtOAc in hexanes) to give the title compound (30 mg, 50%).

### Step 5: ((2R)-5,6-dichloro-7-(1H-pyrrolo[2,3-b]pyridin-4-yl)-2,3-dihydrobenzofuran-2-yl)(piperazin-1-yl)methanone and ((2S)-5,6-dichloro-7-(1H-pyrrolo[2,3-b]pyridin-4-yl)-2,3-dihydrobenzofuran-2-yl)(piperazin-1-yl)methanone

To a solution of tert-butyl 4-(5,6-dichloro-7-(1H-pyrrolo[2,3-b]pyridin-4-yl)-2,3-dihydrobenzofuran-2-carbonyl)piperazine-1-carboxylate (30 mg, 0.1 mmol) in DCM (2 mL) was added TFA (1 mL) at rt The reaction was stirred for 1 hr and concentrated in vacuo. The mixture was treated with HCl (4M in dioxane, 0.3 mL) followed by 10 mL EtOAc, After the solvent was carefully removed, the crude product was concentrated and dried on vacuum to give the title compounds (8 mg). ¹H NMR (400 MHz, CD₃OD, hydrochloric acid salt): δ ppm 9.59 (bs, 1H), 9.51 (bs, 1H), 7.78-7.60 (m, 3H), 6.64 (dd, J=9.5, 2.5 Hz, 1H), 5.46 (dd, J=9.6, 2.4 Hz, 1H), 3.98-3.18 (m, 10H). [M+H] 417.1.

### Example 18

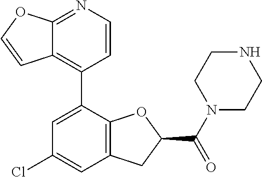 [SMILES: O=C([C@H]1Cc2cc(Cl)cc(-c3ccnc4occc34)c2O1)N1CCNCC1]

### (R)-(5-chloro-7-(furo[2,3-b]pyridin-4-yl)-2,3-dihydrobenzofuran-2-yl)(piperazin-1-yl)methanone

The example was prepared using precursor II, general procedure B with 4-chlorofuro[2,3-b]pyridine and purified by reverse phase HPLC using water (0.1% TFA) and acetonitrile (0.1% TFA) as the eluent followed by using general procedures E and F successively. ¹H NMR (400 MHz, CD₃OD, free base): δ ppm 8.40 (bs, 1H), 7.97 (d, J=2.0 Hz, 1H), 7.57 (d, J=4.8 Hz, 1H), 7.42-7.40 (m, 2H), 7.17 (d, J=2.4 Hz, 1H), 5.82 (dd, J=10.4, 6.8 Hz, 1H), 4.00-3.54 (m, 6H). 3.27-2.95 (m, 4H). [M+H] 383.9.

### Example 19

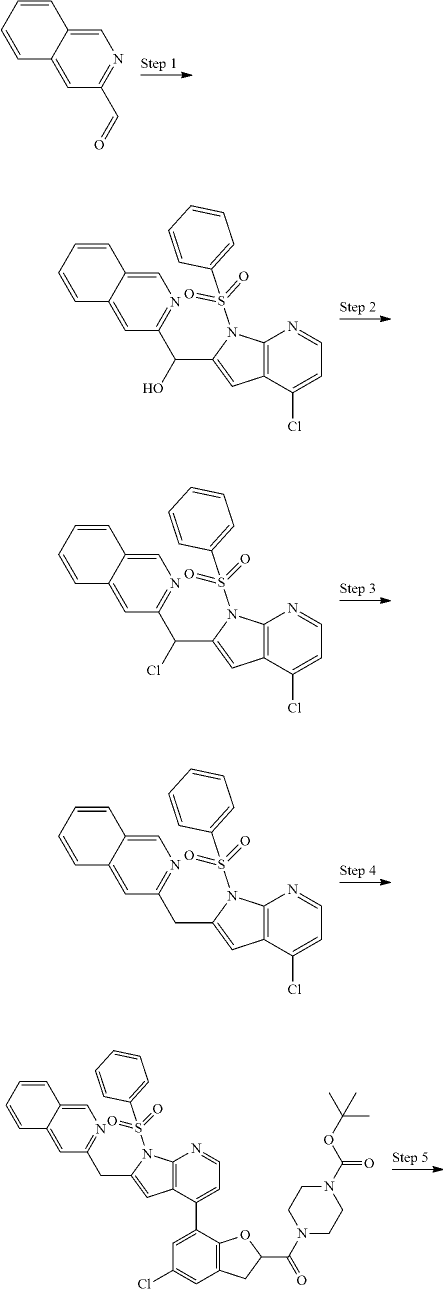 [SMILES: O=Cc1cc2ccccc2cn1 ; O=S(=O)(c1ccccc1)n1c(C(O)c2cc3ccccc3cn2)cc2c(Cl)ccnc21 ; O=S(=O)(c1ccccc1)n1c(C(Cl)c2cc3ccccc3cn2)cc2c(Cl)ccnc21 ; O=S(=O)(c1ccccc1)n1c(Cc2cc3ccccc3cn2)cc2c(Cl)ccnc21 ; CC(C)(C)OC(=O)N1CCN(C(=O)C2Cc3cc(Cl)cc(-c4ccnc5c4cc(Cc4cc6ccccc6cn4)n5S(=O)(=O)c4ccccc4)c3O2)CC1]

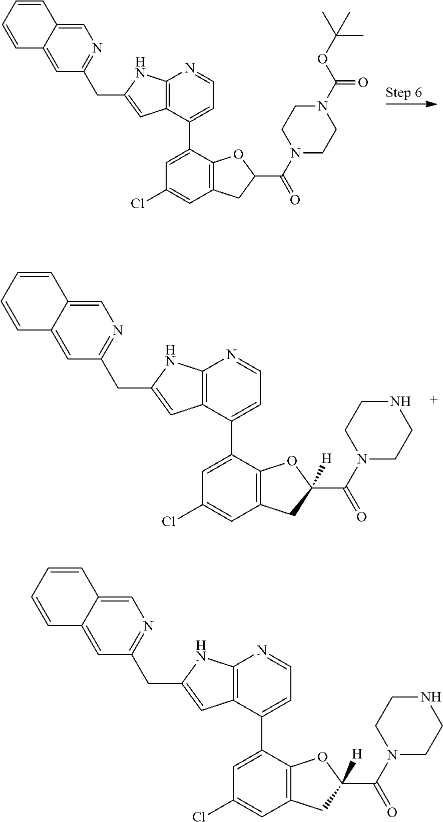 [SMILES: CC(C)(C)OC(=O)N1CCN(C(=O)C2Cc3cc(Cl)cc(-c4ccnc5[nH]c(Cc6cc7ccccc7cn6)cc45)c3O2)CC1 ; O=C([C@@H]1Cc2cc(Cl)cc(-c3ccnc4[nH]c(Cc5cc6ccccc6cn5)cc34)c2O1)N1CCNCC1 ; O=C([C@H]1Cc2cc(Cl)cc(-c3ccnc4[nH]c(Cc5cc6ccccc6cn5)cc34)c2O1)N1CCNCC1]

### Step 1: (4-chloro-1-(phenylsulfonyl)-1H-pyrrolo[2,3-b]pyridin-2-yl)(isoquinolin-3-yl)methanol

To a cooled solution (−45° C.) of 4-chloro-1-(phenylsulfonyl)-1H-pyrrolo[2,3-b]pyridine (270 mg, 0.92 mmol) in anhydrous THF (4 mL) was added a solution of LDA (1M in hexanes, 1.01 mmol). The reaction was stirred at −45° C. for 1 hr, and isoquinoline-3-carbaldehyde (160 mg, 1.01 mmol) was added. The reaction was stirred at −45° C. for 1 hr and quenched with aqueous NH₄Cl. The aqueous layer was extracted with EtOAc. The combined organic layer was washed with water and concentrated. The crude product was purified with column chromatography using 20-50% EtOAc/hexanes to give the title compound (220 mg, 53%).

### Step 2: 3-(chloro(4-chloro-1-(phenylsulfonyl)-1H-pyrrolo[2,3-b]pyridin-2-yl)methyl)isoquinoline

(4-chloro-1-(phenylsulfonyl)-1H-pyrrolo[2,3-b]pyridin-2-yl)(isoquinolin-3-yl)methanol (125 mg, 0.278 mmol) was dissolved in DCM (2 mL) and SOCl₂ (130 mg, 1.05 mmol) was added. The reaction was stirred at RT for 15 min and concentrated to dryness. The crude product was used directly in the next step without further purification.

### Step 3: 3-((4-chloro-1-(phenylsulfonyl)-1H-pyrrolo[2,3-b]pyridin-2-yl)methyl) Isoquinoline

3-(chloro(4-chloro-1-(phenyl sulfonyl)-1H-pyrrolo[2,3-b]pyri din-2-yl)methyl)isoquinoline (119 mg, 0.25 mmol) was dissolved in AcOH (2 mL) and Zn metal (600 mg) was added. The reaction was stirred at RT for 2 hr, and then filtered and washed with DCM. The reaction mixture was concentrated and diluted with aqueous NH₃ (2 mL) and extracted with EtOAc. The organic layer was concentrated and purified with column chromatography using 10-30% EtOAc/hexanes to give the title compound (24 mg, 22%).

### Step 4: tert-butyl 4-(5-chloro-7-(2-(isoquinolin-3-ylmethyl)-1-(phenylsulfonyl)-1H-pyrrolo[2,3-b]pyridin-4-yl)-2,3-dihydrobenzofuran-2-carbonyl)piperazine-1-carboxylate

A mixture of tert-butyl 4-(5-chloro-7-(tributylstannyl)-2,3-dihydrobenzofuran-2-carbonyl)piperazine-1-carboxylate (75 mg, 0.11 mmol), 3-((4-chloro-1-(phenylsulfonyl)-1H-pyrrolo[2,3-b]pyridin-2-yl)methyl)isoquinoline (25 mg, 0.0577 mmol), Tetrakis(triphenylphosphine)palladium(0) (7 mg, 0.006 mmol) in DMF (2 mL) was heated at 130° C. and stirred for 18 hr. The reaction mixture was cooled to room temperature, diluted with water and extracted with EtOAc. The organic layer was washed with water, and concentrated. The crude product was purified by chromatography (0-5% MeOH/EtOAc) to give the title compound (18 mg, 41%).

### Step 5: tert-butyl 4-(5-chloro-7-(2-(isoquinolin-3-ylmethyl)-1H-pyrrolo[2,3-b]pyridin-4-yl)-2,3-dihydrobenzofuran-2-carbonyl)piperazine-1-carboxylate

tert-butyl 4-(5-chloro-7-(2-(isoquinolin-3-ylmethymethyl)-1-(phenyl sulfonyl)-1H-pyrrolo[2,3-b]pyridin-4-yl)-2,3-dihydrobenzofuran-2-carbonyl)piperazine-1-carboxylate (18 mg, 0.023 mmol) was dissolved in THF/MeOH (1:1) (2 mL) and was added with Cs₂CO₃ (29 mg, 0.09 mmol). The reaction was stirred at 55° C. for 24 hr. The reaction mixture was concentrated and extracted with EtOAc. The organic layers were concentrated and purified by chromatography (3-7% MeOH/EtOAc) to give the title compound (12 mg, 81%).

### Step 6: (R)-(5-chloro-7-(2-(isoquinolin-3-ylmethyl)-1H-pyrrolo[2,3-b]pyridin-4-yl)-2,3-dihydrobenzofuran-2-yl)(piperazin-1-yl)methanone and (R)-(5-chloro-7-(2-(isoquinolin-3-ylmethyl)-1H-pyrrolo[2,3-b]pyridin-4-yl)-2,3-dihydrobenzofuran-2-yl)(piperazin-1-yl)methanone

tert-butyl 4-(5-chloro-7-(2-(isoquinolin-3-ylmethyl)-1H-pyrrolo[2,3-b]pyridin-4-yl)-2,3-dihydrobenzofuran-2-carbonyl)piperazine-1-carboxylate (12 mg, 0.019 mmol) was dissolved in DCM (1 mL) and TFA (1 mL) was then added. The reaction was stirred at RT for 1 hr, and then concentrated. The reaction mixture was diluted with NaHCO₃ (1 mL) and extracted with EtOAc. The organic layer was concentrated to dryness to give the title compound (7 mg, 69%). ¹H NMR (400 MHz, DMSO-d₆, hydrochloric acid salt): δ ppm 11.88 (bs, 1H), 9.00 (bs, 1H), 9.41 (bs, 1H), 8.76 (bs, 1H), 8.19 (s, 1H), 7.97 (d, J=7.6 Hz, 1H), 7.84 (t, J=7.6 Hz, 1H), 7.71 (t, J=7.2 Hz, 1H), 7.37 (d, J=8.8 Hz, 1H), 7.29 (s, 1H), 6.42 (s, 1H), 5.77 (t, J=8.4 Hz, 1H), 4.01-2.65 (m, 12H). [M+H] 524.3.

### Example 20

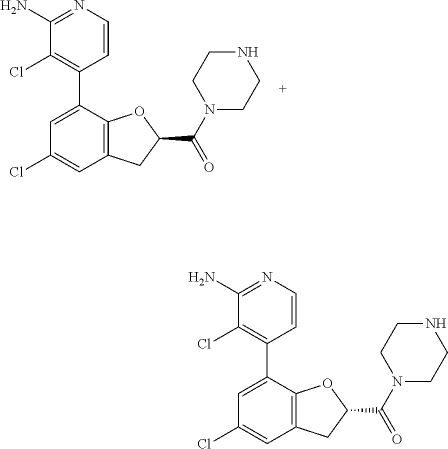 [SMILES: Nc1nccc(-c2cc(Cl)cc3c2O[C@@H](C(=O)N2CCNCC2)C3)c1Cl ; Nc1nccc(-c2cc(Cl)cc3c2O[C@H](C(=O)N2CCNCC2)C3)c1Cl]

### (R)-(7-(2-amino-3-chloropyridin-4-yl)-5-chloro-2,3-dihydrobenzofuran-2-yl)(piperazin-1-yl)methanone and (S)-(7-(2-amino-3-chloropyridin-4-yl)-5-chloro-2,3-dihydrobenzofuran-2-yl)(piperazin-1-yl)methanone

The example was prepared using precursor I (step 5), general procedure C with 4-bromo-3-chloropyridin-2-amine and purified by silica gel chromatography using EtOAc and hexanes (0 to 100% gradient) as eluent followed by using general procedure D. ¹H NMR (400 MHz, CD₃OD, hydrochloric acid salt): δ ppm 7.92 (d, J=6.4 Hz, 1H), 7.41 (s, 1H), 7.27 (s, 1H), 7.05 (d, J=6.4 Hz, 1H), 5.80 (dd, J=9.6, 6.4 Hz, 1H), 3.89-3.17 (m, 10H). [M+H] 393.0.

### Example 21

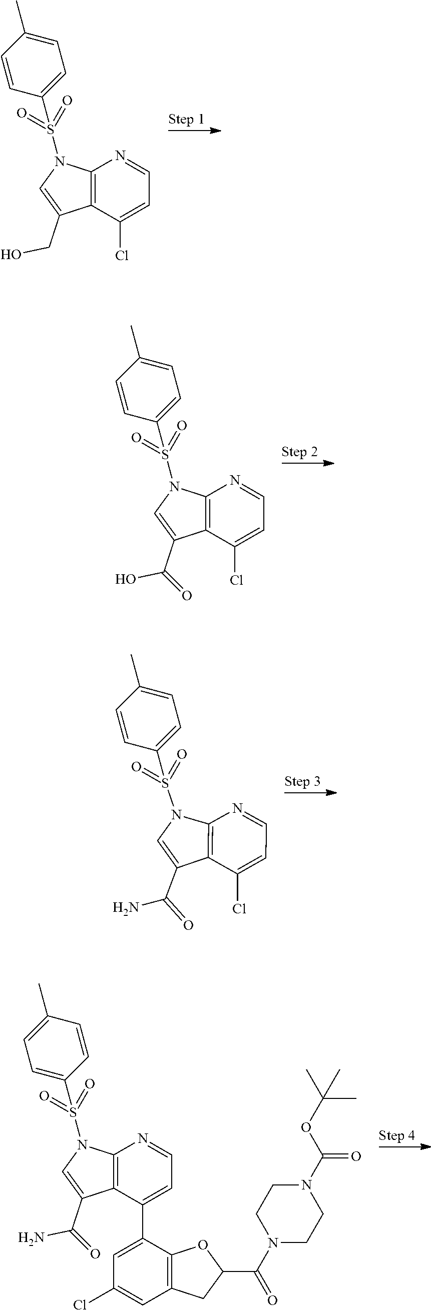

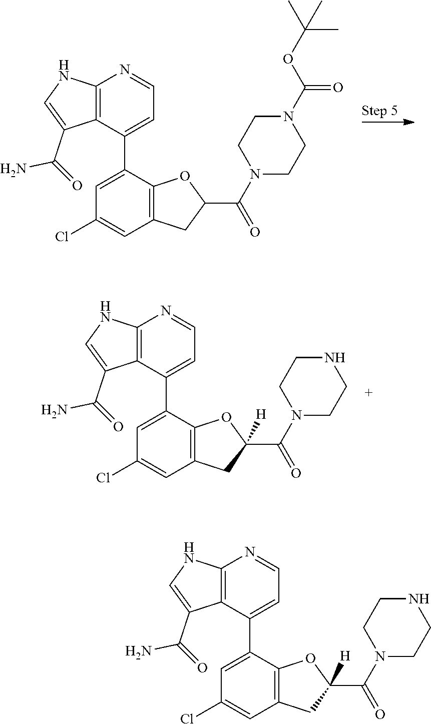 [SMILES: CC(C)(C)OC(=O)N1CCN(C(=O)C2Cc3cc(Cl)cc(-c4ccnc5[nH]cc(C(N)=O)c45)c3O2)CC1 ; NC(=O)c1c[nH]c2nccc(-c3cc(Cl)cc4c3O[C@H](C(=O)N3CCNCC3)C4)c12 ; NC(=O)c1c[nH]c2nccc(-c3cc(Cl)cc4c3O[C@@H](C(=O)N3CCNCC3)C4)c12]

### Step 1: 4-chloro-1-tosyl-1H-pyrrolo[2,3-b]pyridine-3-carboxylic Acid

To a solution of (4-chloro-1-tosyl-1H-pyrrolo[2,3-b]pyridin-3-yl)methanol (200 mg, 0.6 mmol) in acetone (5 mL) and 5% NaHCO₃ solution (5 mL) was added KBr 10 mg, 0.1 eq) and TEMPO (150 mg, 1.2 eq), followed by bleach (5% NaOCl, 4 mL). The mixture was stirred at rt for 2 hr and most of Acetone was removed under reduced pressure. The reaction was washed with MTBE (20 mL×2) and treated with 1N HCl until pH˜1. The aqueous layer was extracted with MTBE (20 mL×2), the combined MTBE layer was washed with brine, dried and concentrated to give the title compound (70 mg) without further purification.

### Step 2: 4-chloro-1-tosyl-1H-pyrrolo[2,3-b]pyridine-3-carboxamide

To a solution of 4-chloro-1-tosyl-1H-pyrrolo[2,3-b]pyridine-3-carboxylic acid (70 mg, 1.9 mmol) in DCM (5 mL) under N₂ at 0° C. was added Thionyl Chloride (35 uL, 2.5 eq). The reaction was stirred at rt for 3 hr before it was concentrated under reduced pressure. The crude was dried under high vac for 30 min and recharged with THF (3 mL) followed by NH₄OH solution (3 mL). The reaction was stirred at rt for 2 hr, extracted with EtOAc, washed with H₂O, Saturated NaHCO₃, brine and dried to give the title compound (40 mg) without further purification.

### Step 3: tert-butyl 4-(7-(3-carbamoyl-1-tosyl-1H-pyrrolo[2,3-b]pyridin-4-yl)-5-chloro-2,3-dihydrobenzofuran-2-carbonyl)piperazine-1-carboxylate

A mixture of tert-butyl 4-(5-chloro-7-(tributylstannyl)-2,3-dihydrobenzofuran-2-carbonyl)piperazine-1-carboxylate (40 mg, 0.23 mmol) and 4-chloro-1-tosyl-1H-pyrrolo[2,3-b]pyridine-3-carboxamide (180 mg, 0.5 mmol) and Pd(PPh₃)₄ (20 mg) in anhydrous DMF (2 mL) under N₂ was heated to 135° C. over night while stirring. The reaction was quenched with H₂O, extracted with EtOAc and washed with H₂O, NaHCO₃ solution and brine. The crude was concentrated and purified with preparative thin layer chromatography (50% EtOAc in DCM) to give the title compound (40 mg, 38%).

### Step 4: tert-butyl 4-(7-(3-carbamoyl-1H-pyrrolo[2,3-b]pyridin-4-yl)-5-chloro-2,3-dihydrobenzofuran-2-carbonyl)piperazine-1-carboxylate

To a solution of tert-butyl 4-(7-(3-carbamoyl-1-tosyl-1H-pyrrolo[2,3-b]pyridin-4-yl)-5-chloro-2,3-dihydrobenzofuran-2-carbonyl)piperazine-1-carboxylate (40 mg, 0.09 mmol) in THF/MeOH (1:1, 3 mL) under N₂ was added Cs₂CO₃ (64 mg, 4 eq). The reaction was stirred at 50 C for 2 hr. The mixture was concentrated under vacuum and extracted with H₂O and EtOAc to give crude intermediate.

### Step 5: (R)-4-(5-chloro-2-(piperazine-1-carbonyl)-2,3-dihydrobenzofuran-7-yl)-1H-pyrrolo[2,3-b]pyridine-3-carboxamide and (S)-4-(5-chloro-2-(piperazine-1-carbonyl)-2,3-dihydrobenzofuran-7-yl)-1H-pyrrolo[2,3-b]pyridine-3-carboxamide

tert-butyl 4-(7-(3-carbamoyl-1H-pyrrolo[2,3-b]pyridin-4-yl)-5-chloro-2,3-dihydrobenzofuran-2-carbonyl)piperazine-1-carboxylate was dissolved in DCM (1 mL) and treated with TFA (0.5 mL) and stirred at rt for 1 hr. The reaction mixture was concentrated and extracted with EtOAc. The organic layers were concentrated and purified by chromatography (3-7% MeOH/EtOAc) to give the title compound (12 mg, 81%). ¹H NMR (400 MHz, DMSO-d₆, hydrochloric acid salt): δ ppm 12.23 (bs, 1H), 9.51 (bs, 1H), 9.15 (bs, 1H), 8.30 (d, J=4.8 Hz, 1H), 7.98 (d, J=2.4 Hz, 1H), 7.27 (s, 1H), 7.17 (1H, D=2.4 Hz, 1H), 5.46 (t, J=9.6 Hz, 1H), 3.77-2.30 (m, 10H). [M+H] 426.1.

### Example 22

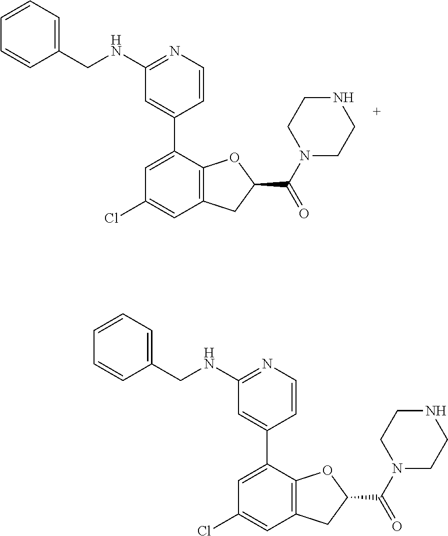 [SMILES: O=C([C@H]1Cc2cc(Cl)cc(-c3ccnc(NCc4ccccc4)c3)c2O1)N1CCNCC1 ; O=C([C@@H]1Cc2cc(Cl)cc(-c3ccnc(NCc4ccccc4)c3)c2O1)N1CCNCC1]

### (R)-(7-(2-(benzylamino)pyridin-4-yl)-5-chloro-2,3-dihydrobenzofuran-2-yl)(piperazin-1-yl)methanone and (S)-(7-(2-(benzylamino)pyridin-4-yl)-5-chloro-2,3-dihydrobenzofuran-2-yl)(piperazin-1-yl)methanone

The example was prepared using precursor I, general procedure C with N-benzyl-4-bromopyridin-2-amine and purified by silica gel chromatography using EtOAc and hexanes (0 to 100% gradient) as eluent followed by using general procedure D. ¹H NMR (400 MHz, CD₃OD, hydrochloric acid salt): δ ppm 7.88 (d, J=6.8 Hz, 1H), 7.52-7.32 (m, 8H), 5.87 (dd, J=10.0, 6.4 Hz, 1H), 4.63 (s, 2H), 3.96-3.52 (m, 10H). [M+H] 449.0.

### Example 23

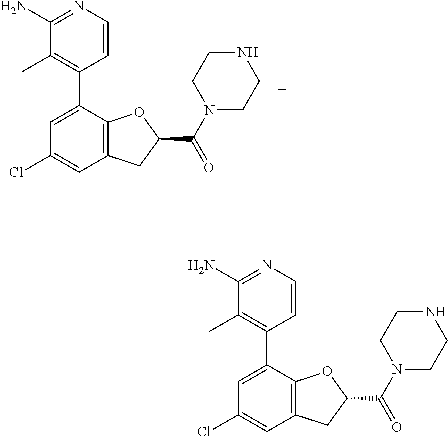 [SMILES: Cc1c(-c2cc(Cl)cc3c2O[C@@H](C(=O)N2CCNCC2)C3)ccnc1N ; Cc1c(-c2cc(Cl)cc3c2O[C@H](C(=O)N2CCNCC2)C3)ccnc1N]

### (R)-(7-(2-amino-3-methylpyridin-4-yl)-5-chloro-2,3-dihydrobenzouran-2-yl)(piperazin-1-yl)methanone and (S)-(7-(2-amino-3-methylpyridin-4-yl)-5-chloro-2,3-dihydrobenzofuran-2-yl)(piperazin-1-yl)methanone

The example was prepared using precursor I, general procedure C with 4-bromo-3-methylpyridin-2-amine and purified by silica gel chromatography using EtOAc and hexanes (0 to 100% gradient) as eluent followed by using general procedure D. ¹H NMR (400 MHz, CD₃OD, hydrochloric acid salt): δ ppm 7.78 (d, J=6.8 Hz, 1H), 7.38 (s, 1H), 7.16 (s, 1H), 6.93 (d, J=6.8 Hz, 1H), 5.80 (t, J=7.0 Hz, 1H), 3.86-3.24 (m, 10H), 2.17 (s, 3H). [M+H] 373.0.

### Example 24

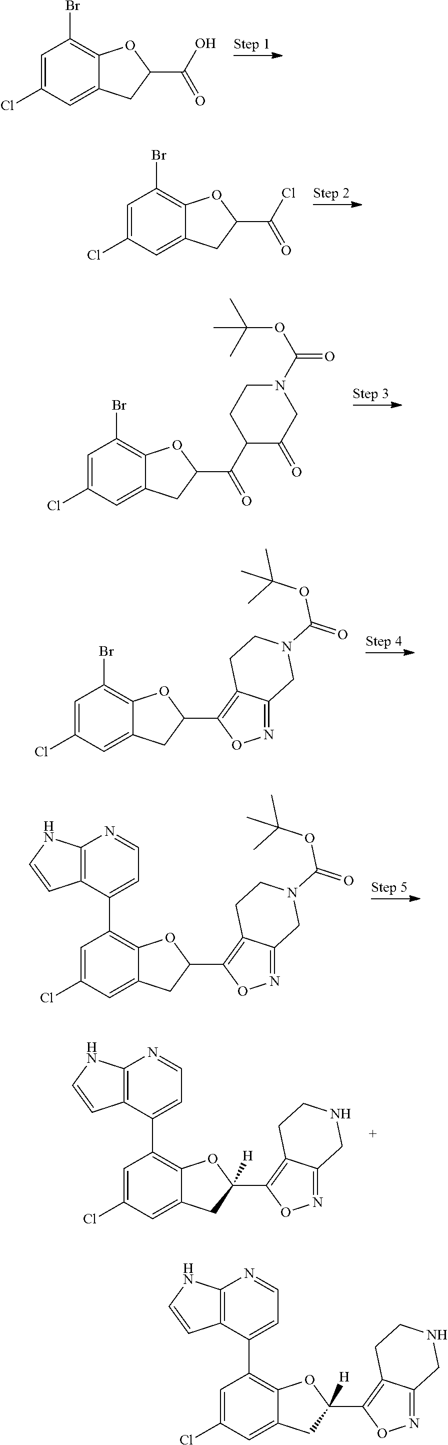

### Step 1: 7-bromo-5-chloro-2,3-dihydrobenzofuran-2-carbonyl Chloride

7-bromo-5-chloro-2,3-dihydrobenzofuran-2-carboxylic acid (2.77 g, 10 mmol) was dissolved in THF (50 mL) at 0° C. and oxalyl chloride (1.28 mL, 15 mmol) was added dropwise. The reaction mixture was stirred at rt for 1 h and concentrated under vacuum to give the title compound as a thick oil which was used in the next step without further purification.

### Step 2: tert-butyl 4-(7-bromo-5-chloro-2,3-dihydrobenzofuran-2-carbonyl)-3-oxopiperidine-1-carboxylate

To a cooled solution (−78° C.) of tert-butyl 3-oxopiperidine-1-carboxylate (1.146 g, 5.75 mmol) in anhydrous THF (15 mL) was added a solution of LDA (5.93 mL, 1M in hexanes, 5.93 mmol). After stirring at −78° C. for 30 min, the reaction was added 7-bromo-5-chloro-2,3-dihydrobenzofuran-2-carbonyl chloride (1 g, in 2 mL THF, 3.3 mmol), stirred at −78° C. for 30 min and then warmed to 0° C. The reaction quenched with aqueous NH₄Cl at 0° C. and the aqueous layer was extracted with EtOAc. The combined organic layers were concentrated. The crude product was purified with column chromatography using 5-25% EtOAc/hexanes to give the title compound (410 mg, 27%).

### Step 3: tert-butyl 3-(7-bromo-5-chloro-2,3-dihydrobenzofuran-2-yl)-4,5-dihydroisoxazolo[3,4-c]pyridine-6(7H)-carboxylate

tert-butyl 4-(7-bromo-5-chloro-2,3-dihydrobenzofuran-2-carbonyl)-3-oxopiperidine-1-carboxylate (100 mg, 0.218 mmol) was dissolved in EtOH (2 mL) and was added NH₂OH—HCl salt (32 mg, 0.46 mmol) and NaOAc (50 mg, 0.6 mmol). The reaction was heated at 78° C. and stirred for 3 hrs. The reaction mixture was cooled and concentrated. The reaction mixture was diluted with water and extracted with EtOAc. The organic layers were concentrated and the crude product was purified by chromatography (20-40% EtOAc/hexanes) to give the title compound (28 mg, 28%).

### Step 4: tert-butyl 3-(5-chloro-7-(1H-pyrrolo[2,3-b]pyridin-4-yl)-2,3-dihydrobenzofuran-2-yl)-4,5-dihydroisoxazolo[3,4-c]pyridine-6(7H)-carboxylate

A mixture of compound tert-butyl 3-(7-bromo-5-chloro-2,3-dihydrobenzofuran-2-yl)-4,5-dihydroisoxazolo[3,4-c]pyridine-6(7H)-carboxylate (28 mg, 0.0615 mmol), 4-(4,4,5,5-tetramethyl-1,3,2-dioxaborolan-2-yl)-1H-pyrrolo[2,3-b]pyridine (23 mg, 0.092 mmol), Tetrakis(triphenylphosphine)palladium(0) (7.6 mg, 0.0066 mmol), and K₂CO₃ (27 mg, 0.2 mmol) in dioxane (1.5 mL) and water (0.5 mL) was heated at 100° C. and stirred for 3 hr. The reaction mixture was cooled to room temperature, diluted with water and extracted with EtOAc. The organic layers were concentrated. The crude product was purified by chromatography (25-65% EtOAc/hexanes) to give 23 (9.1 mg, 30%).

### Step 5: (R)-3-(5-chloro-7-(1H-pyrrolo[2,3-b]pyridin-4-yl)-2,3-dihydrobenzofuran-2-yl)-4,5,6,7-tetrahydroisoxazolo[3,4-c]pyridine and (S)-3-(5-chloro-7-(1H-pyrrolo[2,3-b]pyridin-4-yl)-2,3-dihydrobenzofuran-2-yl)-4,5,6,7-tetrahydroisoxazolo[3,4-c]pyridine

tert-butyl 3-(5-chloro-7-(1H-pyrrolo[2,3-b]pyridin-4-yl)-2,3-dihydrobenzofuran-2-yl)-4,5-dihydroisoxazolo[3,4-c]pyridine-6(7H)-carboxylate (9.1 mg, 0.0185 mmol) was dissolved in DCM (1 mL) and TFA (1 mL) was then added. The reaction was stirred at RT for 1 hr, and concentrated. The reaction mixture was treated with HCl (4M, in dioxane, 0.3 mL) and ether (2 mL) was added. The title compound was precipitated out as the HCl salt (5.9 mg, 78%). ¹H NMR (400 MHz, CD₃OD, hydrochloric acid salt): δ ppm 12.05 (bs, 1H), 9.62 (bs, 1H), 8.28 (d, J=5.2 Hz, 1H), 7.55 (d, J=3.2 Hz, 1H), 7.54 (d, J=2.4 Hz, 1H), 7.43 (d, J=5.2 Hz, 1H), 6.42 (d, J=1.6 Hz, 1H), 6.21 (t, J=9.6 Hz, 1H), 3.52-2.40 (m, 8H). [M+H] 393.1.

### Example 25

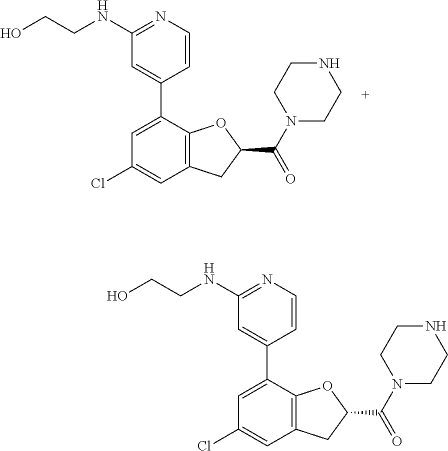 [SMILES: O=C([C@H]1Cc2cc(Cl)cc(-c3ccnc(NCCO)c3)c2O1)N1CCNCC1 ; O=C([C@@H]1Cc2cc(Cl)cc(-c3ccnc(NCCO)c3)c2O1)N1CCNCC1]

### (R)-(5-chloro-7-(2-((2-hydroxyethyl)amino)pyridin-4-yl)-2,3-dihydrobenzofuran-2-yl)(piperazin-1-yl)methanone and (S)-(5-chloro-7-(2-((2-hydroxyethyl)amino)pyridin-4-yl)-2,3-dihydrobenzofuran-2-yl)(piperazin-1-yl)methanone

The example was prepared using precursor I, general procedure C with 4-bromo-N-(2-((tert-butyldimethylsilyl)oxy)ethyl)pyridin-2-amine and purified by silica gel chromatography using EtOAc and hexanes (0 to 100% gradient) as eluent followed by using general procedure D. ¹H NMR (400 MHz, CD₃OD, hydrochloric acid salt): δ ppm 7.86 (d, J=6.8 Hz, 1H), 7.51 (d, J=1.6 Hz, 1H), 7.50 (d, J=2.0 Hz, 1H), 7.41 (d, J=2.0 Hz, 1H), 7.29 (dd, J=6.8, 1.6 Hz, 1H), 5.89 (dd, J=9.6, 7.2 Hz, 1H), 4.63 (s, 2H), 4.02-3.24 (m, 14H). [M+H] 403.0.

### Example 26

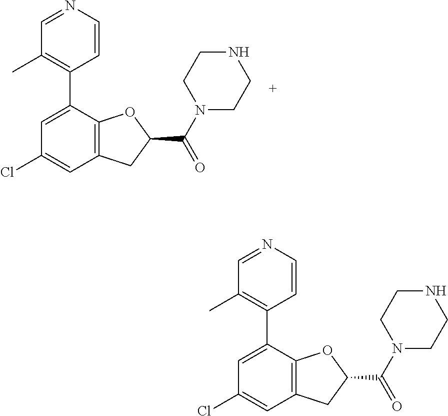 [SMILES: Cc1cnccc1-c1cc(Cl)cc2c1O[C@@H](C(=O)N1CCNCC1)C2 ; Cc1cnccc1-c1cc(Cl)cc2c1O[C@H](C(=O)N1CCNCC1)C2]

### (R)-(5-chloro-7-(3-methylpyridin-4-yl)-2,3-dihydrobenzofuran-2-yl)(piperazin-1-yl)methanone and (S)-(5-chloro-7-(3-methylpyridin-4-yl)-2,3-dihydrobenzofuran-2-yl)(piperazin-1-yl)methanone

The example was prepared using precursor I, general procedure C 4-bromo-3-methylpyridine and purified by silica gel chromatography using EtOAc and hexanes (0 to 100% gradient) as eluent followed by using general procedure D. ¹H NMR (400 MHz, CD₃OD, hydrochloric acid salt): δ ppm 8.84 (s, 1H), 8.73 (d, J=6.0 Hz, 1H), 8.05 (d, J=6.0 Hz, 1H), 7.46 (s, 1H), 7.28 (s, 1H), 5.85 (dd, J=10.0, 6.4 Hz, 1H), 3.84-3.17 (m, 10H), 2.52 (s, 3H). [M+H] 373.0.

### Example 27

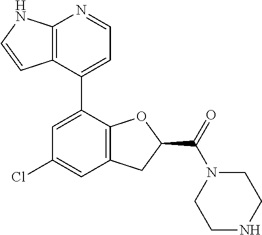 [SMILES: O=C([C@H]1Cc2cc(Cl)cc(-c3ccnc4[nH]ccc34)c2O1)N1CCNCC1]

### (R)-(5-chloro-7-(1H-pyrrolo[2,3-b]pyridin-4-yl)-2,3-dihydrobenzofuran-2-yl)(piperazin-1-yl)methanone

The example was prepared as the HCl salt using precursor II, general procedures C and D with tert-butyl (R)-4-(7-bromo-5-chloro-2,3-dihydrobenzofuran-2-carbonyl)piperazine-1-carboxylate and 4-(4,4,5,5-tetramethyl-1,3,2-dioxaborolan-2-yl)-1H-pyrrolo[2,3-b]pyridine. ¹H NMR (400 MHz, CD₃OD) δ 8.44 (d, J=6.2 Hz, 1H), 7.81 (d, J=6.1 Hz, 1H), 7.74 (d, J=3.6 Hz, 1H), 7.56 (d, J=2.2 Hz, 1H), 7.47 (s, 1H), 7.03 (d, J=3.6 Hz, 1H), 5.91-5.81 (m, 1H), 4.02-3.76 (m, 4H), 3.77-3.69 (m, 1H), 3.61-3.54 (m, 1H), 3.43-3.04 (m, 4H). [M+H⁺] 383.1

### Example 28

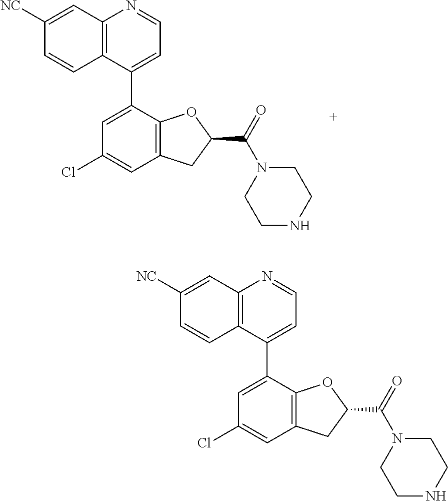 [SMILES: N#Cc1ccc2c(-c3cc(Cl)cc4c3O[C@@H](C(=O)N3CCNCC3)C4)ccnc2c1 ; N#Cc1ccc2c(-c3cc(Cl)cc4c3O[C@H](C(=O)N3CCNCC3)C4)ccnc2c1]

### (R)-4-(5-chloro-2-(piperazine-1-carbonyl)-2,3-dihydrobenzofuran-7-yl)quinoline-7-carbonitrile and and (S)-4-(5-chloro-2-(piperazine-1-carbonyl)-2,3-dihydrobenzofuran-7-yl)quinoline-7-carbonitrile

methyl 4-(2-(4-(tert-butoxycarbonyl)piperazine-1-carbonyl)-5-chloro-2,3-dihydrobenzofuran-7-yl)quinoline-7-carboxylate was prepared from methyl 4-chloroquinoline-7-carboxylate using general procedure C. To methyl 4-[2-(4-tert-butoxycarbonylpiperazine-1-carbonyl)-5-chloro-2,3-dihydrobenzofuran-7-yl]quinoline-7-carboxylate (400 mg, 0.72 mmol) in 1,4-Dioxane (0.80 mL) is added 4M LiOH (0.2 mL, 0.8 mmol). The mixture was stirred overnight before being diluted with water (5 mL) and TBME (5 mL). The aqueous layer was separated and the organic layer was extracted with water (2 mL). The combined aqueous layers were diluted with EtOAc (10 mL) and acidified to pH<2 with 3M HCl. The aqueous layer was extracted with EtOAc (2×5 mL). The combined organic layers were dried over sodium sulfate, filtered, and concentrated in vacuo to afford 4-[2-(4-tert-butoxycarbonylpiperazine-1-carbonyl)-5-chloro-2,3-dihydrobenzofuran-7-yl]quinoline-7-carboxylic acid. To 4-[2-(4-tert-butoxycarbonylpiperazine-1-carbonyl)-5-chloro-2,3-dihydrobenzofuran-7-yl]quinoline-7-carboxylic acid (100 mg, 0.1900 mmol) and N,N-diisopropylethylamine (0.1 mL, 0.56 mmol) in DMF (0.46 mL) was added HATU (71 mg, 0.19 mmol). The mixture was stirred for 5 min, then solid ammonium chloride (19.7 mg, 0.37 mmol) was added. After 2 h, the mixture was concentrated in vacuo and the residue was purified by flash column chromatography (0-20% MeOH in DCM) to afford the primary amide (69 mg, 69% yield). To tert-butyl 4-(7-(7-carbamoylquinolin-4-yl)-5-chloro-2,3-dihydrobenzofuran-2-carbonyl)piperazine-1-carboxylate (27 mg, 0.05 mmol, 1.0 eq) in DCM (1 mL) is added trifluoroacetic anhydride (15.8 mg, 0.075 mmol, 1.5 eq) and pyridine (11.9 mg, 0.151 mmol, 3.0 eq). The mixture is allowed to stir overnight before being diluted with sat. aq. sodium bicarbonate (5 mL) and EtOAc (5 mL). The organic layer was dried over sodium sulfate, filtered, and concentrated in vacuo. The residue was purified by flash column chromatography (20-100% EtOAc in hexanes) to afford the intermediate nitrile (18.5 mg, 70% yield). The example was prepared as the HCl salt from this intermediate by deprotection according to general procedure D. ¹H NMR (400 MHz, CD₃OD, hydrochloric acid salt): δ ppm 9.28 (d, J=5.2 Hz, 1H), 8.67-8.61 (m, 1H), 8.38-8.32 (m, 1H), 8.07 (d, J=5.1 Hz, 1H), 8.02 (dd, J=8.8, 1.6 Hz, 1H), 7.55-7.51 (m, 1H), 7.41 (d, J=2.2 Hz, 1H), 5.80 (t, J=8.3 Hz, 1H), 3.90-3.51 (m, 6H), 3.32-2.91 (m, 4H). LCMS: [M+H⁺]=419.0.

### Example 29

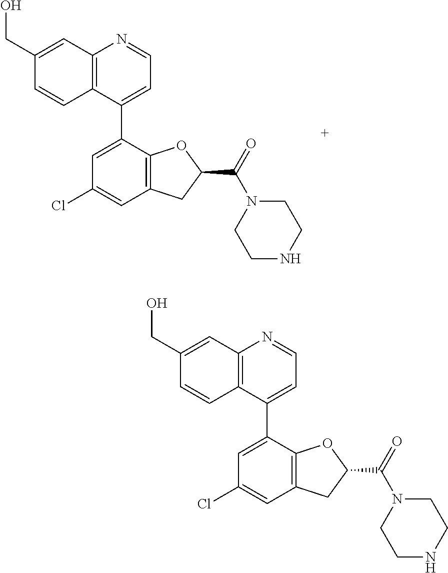 [SMILES: O=C([C@H]1Cc2cc(Cl)cc(-c3ccnc4cc(CO)ccc34)c2O1)N1CCNCC1 ; O=C([C@@H]1Cc2cc(Cl)cc(-c3ccnc4cc(CO)ccc34)c2O1)N1CCNCC1]

### (R)-(5-chloro-7-(7-(hydroxymethyl)quinolin-4-yl)-2,3-dihydrobenzofuran-2-yl)(piperazin-1-yl)methanone and (S)-(5-chloro-7-(7-(hydroxymethyl)quinolin-4-yl)-2,3-dihydrobenzofuran-2-yl)(piperazin-1-yl)methanone

The example was prepared using precursor I, general procedures C and D as the HCl salt with (4-chloroquinolin-7-yl)methanol. ¹H NMR (400 MHz, CD₃OD, hydrochloric acid salt): δ ppm 9.23-9.17 (m, 1H), 8.28 (s, 1H), 8.25 (d, J=9.1 Hz, 1H), 8.12 (s, 1H), 7.90 (d, J=8.7 Hz, 1H), 7.55 (s, 1H), 7.45 (d, J=2.1 Hz, 1H), 5.81 (t, J=8.4 Hz, 1H), 4.98 (s, 2H), 3.94-3.63 (m, 6H), 3.27-3.11 (m, J=15.3 Hz, 3H), 3.00-2.90 (m, 1H). LCMS: [M+H⁺]=424.0.

### Example 30

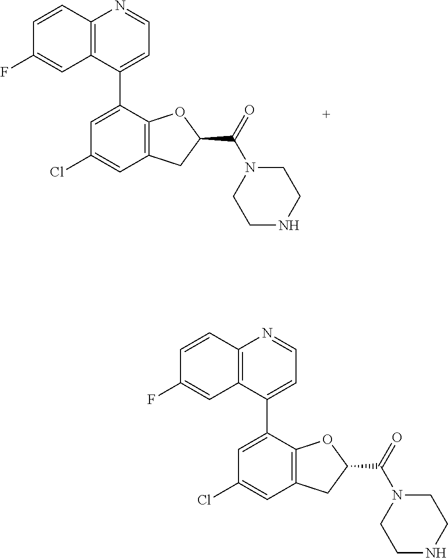 [SMILES: O=C([C@H]1Cc2cc(Cl)cc(-c3ccnc4ccc(F)cc34)c2O1)N1CCNCC1 ; O=C([C@@H]1Cc2cc(Cl)cc(-c3ccnc4ccc(F)cc34)c2O1)N1CCNCC1]

### (R)-(5-chloro-7-(6-fluoroquinolin-4-yl)-2,3-dihydrobenzofuran-2-yl)(piperazin-1-yl)methanone and (S)-(5-chloro-7-(6-fluoroquinolin-4-yl)-2,3-dihydrobenzofuran-2-yl)(piperazin-1-yl)methanone

The example was prepared using precursor I, general procedures C and D as the HCl salt with 4-chloro-6-fluoroquinoline. ¹H NMR (400 MHz, CD₃OD, hydrochloric acid salt): δ ppm 9.23 (d, J=5.6 Hz, 1H), 8.37 (dd, J=9.4, 4.8 Hz, 1H), 8.15 (d, J=5.1 Hz, 1H), 8.09-8.01 (m, 1H), 7.96 (br s, 1H), 7.59-7.54 (m, 1H), 7.45 (d, J=2.2 Hz, 1H), 5.84 (dd, J=9.4, 6.7 Hz, 1H), 3.85 (s, 2H), 3.80-3.66 (m, 4H), 3.29-3.14 (m, 3H), 3.05 (s, 1H). LCMS: [M+H⁺]=412.0.

### Example 31

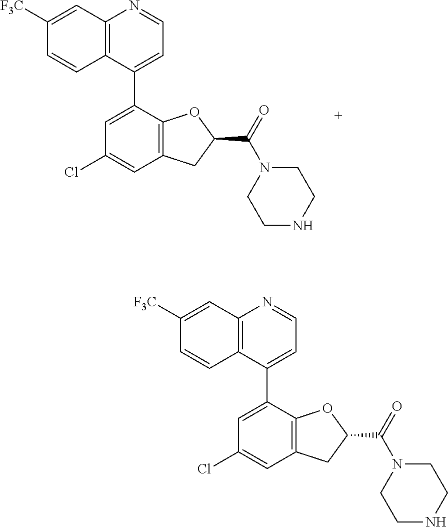 [SMILES: O=C([C@H]1Cc2cc(Cl)cc(-c3ccnc4cc(C(F)(F)F)ccc34)c2O1)N1CCNCC1 ; O=C([C@@H]1Cc2cc(Cl)cc(-c3ccnc4cc(C(F)(F)F)ccc34)c2O1)N1CCNCC1]

### (R)-(5-chloro-7-(7-(trifluoromethyl)quinolin-4-yl)-2,3-dihydrobenzofuran-2-yl)(piperazin-1-yl)methanone and (S)-(5-chloro-7-(7-(trifluoromethyl)quinolin-4-yl)-2,3-dihydrobenzofuran-2-yl)(piperazin-1-yl)methanone

The example was prepared using precursor I, general procedures C and D as the HCl salt with 4-chloro-7-(trifluoromethyl)quinoline. ¹H NMR (400 MHz, CD₃OD, hydrochloric acid salt): δ ppm 9.29 (d, J=5.2 Hz, 1H), 8.53 (s, 1H), 8.44 (s, 1H), 8.09 (d, J=4.7 Hz, 1H), 8.05 (d, J=9.0 Hz, 1H), 7.55-7.52 (m, 1H), 7.43 (d, J=2.2 Hz, 1H), 5.85-5.78 (m, 1H), 3.93-3.82 (m, 1H), 3.81-3.65 (m, 5H), 3.29-3.10 (m, 3H), 3.02-2.93 (m, 1H). LCMS: [M+H⁺]=482.0.

### Example 32

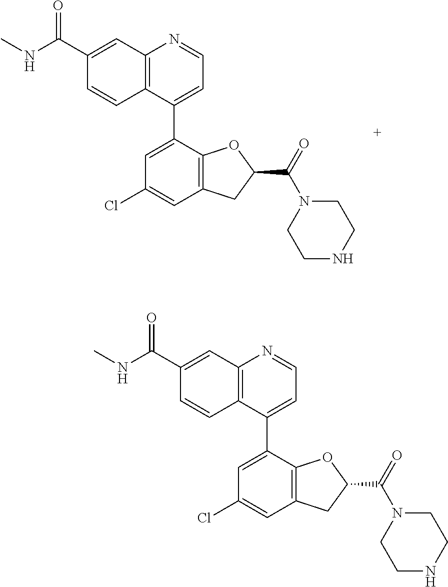 [SMILES: CNC(=O)c1ccc2c(-c3cc(Cl)cc4c3O[C@@H](C(=O)N3CCNCC3)C4)ccnc2c1 ; CNC(=O)c1ccc2c(-c3cc(Cl)cc4c3O[C@H](C(=O)N3CCNCC3)C4)ccnc2c1]

### (R)-4-(5-chloro-2-(piperazine-1-carbonyl)-2,3-dihydrobenzofuran-7-yl)-N-methylquinoline-7-carboxamide and (S)-4-(5-chloro-2-(piperazine-1-carbonyl)-2,3-dihydrobenzofuran-7-yl)-N-methylquinoline-7-carboxamide

To 4-[2-(4-tert-butoxycarbonylpiperazine-1-carbonyl)-5-chloro-2,3-dihydrobenzofuran-7-yl]quinoline-7-carboxylic acid (100 mg, 0.1900 mmol) and N,N-diisopropylethylamine (0.1 mL, 0.56 mmol) in DMF (0.46 mL) was added HATU (71 mg, 0.19 mmol). The mixture was stirred for 5 min, then 33% w/w methylamine in ethanol (0.1 mL) was added. After 2 h, the mixture was concentrated in vacuo and the residue was purified by flash column chromatography (0-20% MeOH in DCM) to afford the amide (75 mg, 73% yield). The example was prepared as the HCl salt from this intermediate by deprotection according to general procedure D. ¹H NMR (400 MHz, CD₃OD, hydrochloric acid salt): δ ppm 9.29 (d, J=5.5 Hz, 1H), 8.66 (d, J=1.3 Hz, 1H), 8.34 (d, J=8.9 Hz, 1H), 8.23 (dd, J=8.9, 1.7 Hz, 1H), 8.14 (d, J=5.3 Hz, 1H), 8.04 (s, 1H), 7.56 (d, J=2.2 Hz, 1H), 7.46 (d, J=2.1 Hz, 1H), 5.85-5.79 (m, 1H), 3.94-3.82 (m, 1H), 3.82-3.62 (m, 5H), 3.29-3.06 (m, 3H), 3.03 (s, 3H), 2.95-2.87 (m, 1H). LCMS: [M+H⁺]=451.0.

### Example 33

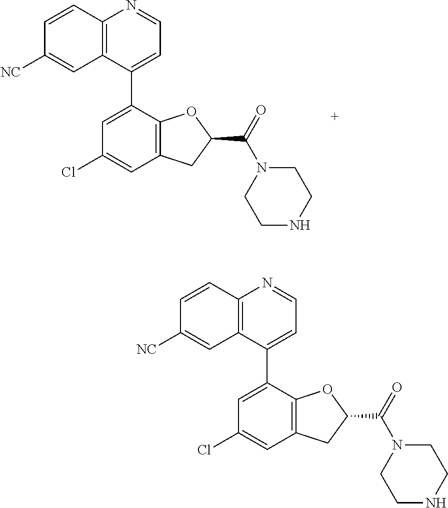 [SMILES: N#Cc1ccc2nccc(-c3cc(Cl)cc4c3O[C@@H](C(=O)N3CCNCC3)C4)c2c1 ; N#Cc1ccc2nccc(-c3cc(Cl)cc4c3O[C@H](C(=O)N3CCNCC3)C4)c2c1]

### (R)-4-(5-chloro-2-(piperazine-1-carbonyl)-2,3-dihydrobenzofuran-7-yl)quinoline-6-carbonitrile and (S)-4-(5-chloro-2-(piperazine-1-carbonyl)-2,3-dihydrobenzofuran-7-yl)quinoline-6-carbonitrile

The example was prepared using precursor I, general procedures C and D as the HCl salt with 4-chloroquinoline-6-carbonitrile. ¹H NMR (400 MHz, CD₃OD, hydrochloric acid salt): δ ppm 9.27-9.22 (m, 1H), 8.93-8.62 (m, 1H), 8.33 (d, J=9.0 Hz, 1H), 8.22 (s, 1H), 7.97 (s, 1H), 7.57-7.50 (m, 1H), 7.44 (s, 1H), 5.86-5.76 (m, 1H), 3.93 (s, 1H), 3.89-3.64 (m, 5H), 3.31-3.12 (m, 3H), 3.11-2.94 (m, 1H). LCMS: [M+H^{+]}=419.0.

### Example 34

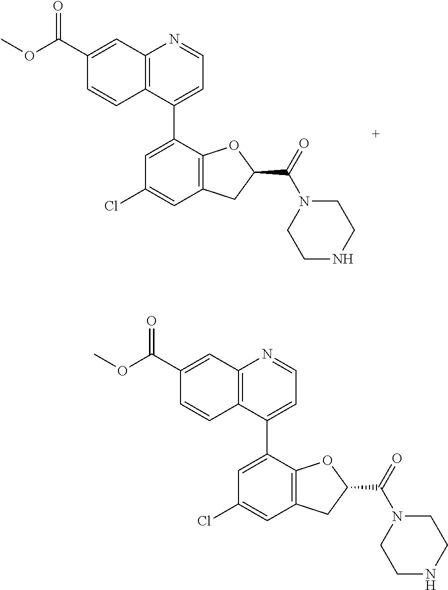 [SMILES: COC(=O)c1ccc2c(-c3cc(Cl)cc4c3O[C@@H](C(=O)N3CCNCC3)C4)ccnc2c1 ; COC(=O)c1ccc2c(-c3cc(Cl)cc4c3O[C@H](C(=O)N3CCNCC3)C4)ccnc2c1]

### Methyl (R)-4-5-chloro-2-piperazine-1-carbonyl)-2,3-dihydrobenzouran-7-yl)quinoline-7-carboxylate and methyl (S)-4-(5-chloro-2-(piperazine-1-carbonyl)-2,3-dihydrobenzofuran-7-yl)quinoline-7-carboxylate

methyl 4-(2-(4-(tert-butoxycarbonyl)piperazine-1-carbonyl)-5-chloro-2,3-dihydrobenzofuran-7-yl)quinoline-7-carboxylate was prepared from methyl 4-chloroquinoline-7-carboxylate using general procedure C. The example was prepared as the HCl salt from this intermediate by deprotection according to general procedure D. ¹H NMR (400 MHz, CD₃OD, hydrochloric acid salt): δ ppm 9.33 (d, J=5.5 Hz, 1H), 8.88 (s, 1H), 8.41 (s, 2H), 8.22-8.17 (m, 1H), 7.58-7.54 (m, 1H), 7.46 (d, J=2.2 Hz, 1H), 5.83 (dd, J=9.7, 6.7 Hz, 1H), 4.06 (s, 3H), 3.93-3.55 (m, 6H), 3.28-2.93 (m, 4H). LCMS: [M+H^{+]}=452.0.

### Example 35

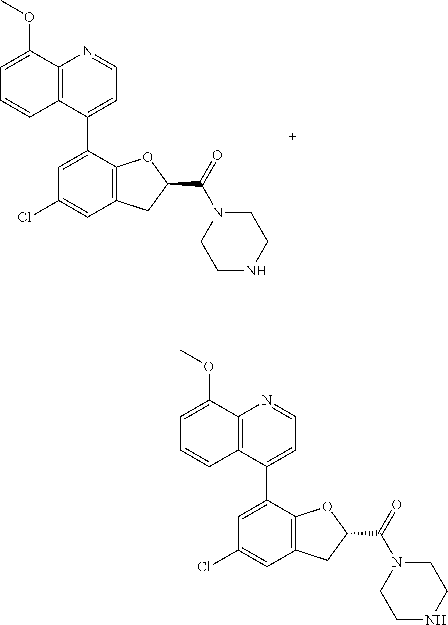 [SMILES: COc1cccc2c(-c3cc(Cl)cc4c3O[C@@H](C(=O)N3CCNCC3)C4)ccnc12 ; COc1cccc2c(-c3cc(Cl)cc4c3O[C@H](C(=O)N3CCNCC3)C4)ccnc12]

### (R)-(5-chloro-7-(8-methoxyquinolin-1-yl)-2,3-dihydrobenzouran-2-yl)(piperazin-1-yl)methanone and (S)-(5-chloro-7-(8-methoxyquinolin-4-yl)-2,3-dihydrobenzofuran-2-yl)(piperazin-1-yl)methanone

The example was prepared using precursor I, general procedures C and D as the HCl salt with 4-chloro-8-methoxyquinoline. ¹H NMR (400 MHz, CD₃OD, hydrochloric acid salt): δ ppm 9.11 (d, J=5.7 Hz, 1H), 8.21 (d, J=5.4 Hz, 1H), 7.96-7.86 (m, 1H), 7.79 (d, J=8.4 Hz, 1H), 7.71 (d, J=8.0 Hz, 1H), 7.60-7.53 (m, 1H), 7.44 (d, J=2.2 Hz, 1H), 5.82 (t, J=8.2 Hz, 1H), 4.27 (s, 3H), 3.93-3.66 (m, 6H), 3.29-3.11 (m, 3H), 3.05-2.93 (m, 1H). LCMS: [M+H⁺]=424.0.

### Example 36

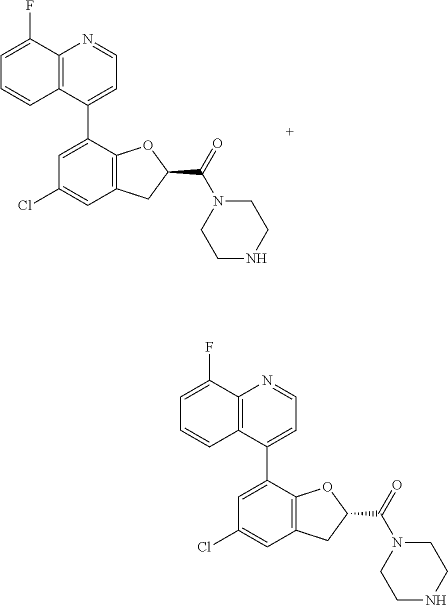 [SMILES: O=C([C@H]1Cc2cc(Cl)cc(-c3ccnc4c(F)cccc34)c2O1)N1CCNCC1 ; O=C([C@@H]1Cc2cc(Cl)cc(-c3ccnc4c(F)cccc34)c2O1)N1CCNCC1]

### (R)-(5-chloro-7-(8-fluoroquinolin-4-yl)-2,3-dihydrobenzofuran-2-yl)(piperazin-1-yl)methanone and (S)-(5-chloro-7-(8-fluoroquinolin-4-yl)-2,3-dihydrobenzofuran-2-yl)(piperazin-1-yl)methanone

The example was prepared using precursor I, general procedures C and D as the HCl salt with 4-chloro-8-fluoroquinoline. ¹H NMR (400 MHz, CD₃OD, hydrochloric acid salt): δ ppm 9.27-9.20 (m, 1H), 8.23-8.13 (m, 1H), 8.07 (s, 1H), 8.02-7.85 (m, 2H), 7.58-7.53 (m, 1H), 7.48-7.43 (m, 1H), 5.88-5.76 (m, 1H), 3.93-3.66 (m, 6H), 3.28-3.11 (m, 3H), 2.97 (s, 1H). LCMS: [M+H⁺]=412.0.

### Example 37

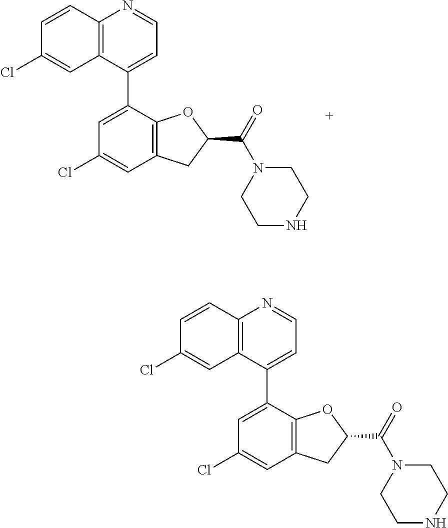 [SMILES: O=C([C@H]1Cc2cc(Cl)cc(-c3ccnc4ccc(Cl)cc34)c2O1)N1CCNCC1 ; O=C([C@@H]1Cc2cc(Cl)cc(-c3ccnc4ccc(Cl)cc34)c2O1)N1CCNCC1]

### (R)-(5-chloro-7-(6-chloroquinolin-4-yl)-2,3-dihydrobenzofuran-2-yl)(piperazin-1-yl)methanone and (S)-(5-chloro-7-(6-chloroquinolin-4-yl)-2,3-dihydrobenzofuran-2-yl)(piperazin-1-yl)methanone

The example was prepared using precursor I, general procedures C and D as the HCl salt with 4,6-dichloroquinoline. ¹H NMR (400 MHz, CD₃OD, hydrochloric acid salt): δ ppm 9.20-9.13 (m, 1H), 8.25 (d, J=9.2 Hz, 1H), 8.19-8.12 (m, 1H), 8.12-8.06 (m, 1H), 8.06-7.96 (m, 1H), 7.55 (s, 1H), 7.42 (s, 1H), 5.83-5.75 (m, 1H), 3.92-3.81 (m, 1H), 3.79-3.66 (m, 5H), 3.29-3.10 (m, 3H), 3.01-2.91 (m, 1H). LCMS: [M+H^{+]}=428.0.

### Example 38

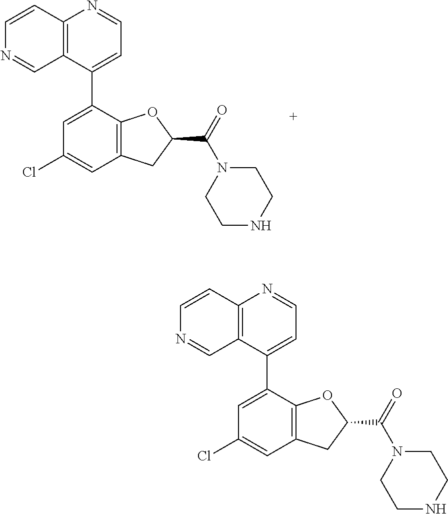 [SMILES: O=C([C@H]1Cc2cc(Cl)cc(-c3ccnc4ccncc34)c2O1)N1CCNCC1 ; O=C([C@@H]1Cc2cc(Cl)cc(-c3ccnc4ccncc34)c2O1)N1CCNCC1]

### (R)-(5-chloro-7-(1,6-naphthyridin-4-yl)-2,3-dihydrobenzofuran-2-yl)(piperazin-1-yl)methanone and (S)-(5-chloro-7-(1,6-naphthyridin-4-yl)-2,3-dihydrobenzofuran-2-yl)(piperazin-1-yl)methanone

The example was prepared using precursor I, general procedures B and D as the HCl salt with 4-chloro-1,6-naphthyridine. ¹H NMR (400 MHz, CD₃OD, hydrochloric acid salt): δ ppm 10.17 (s, 1H), 9.51 (d, J=4.6 Hz, 1H), 8.85 (d, J=6.7 Hz, 1H), 8.57 (d, J=6.6 Hz, 1H), 8.00 (d, J=4.6 Hz, 1H), 7.71-7.60 (m, 1H), 7.59-7.54 (m, 1H), 7.54-7.53 (m, 1H), 7.51 (d, J=2.2 Hz, 1H), 5.83 (dd, J=10.7, 5.0 Hz, 1H), 4.05-3.91 (m, 1H), 3.89-3.71 (m, 5H), 3.61-3.41 (m, 1H), 3.34-3.17 (m, 3H). LCMS: [M+H⁺]=395.0.

### Example 39

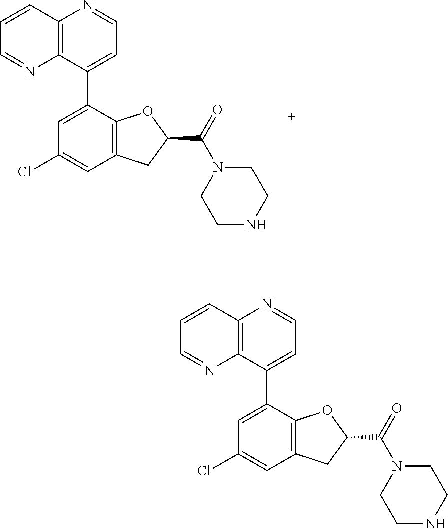 [SMILES: O=C([C@H]1Cc2cc(Cl)cc(-c3ccnc4cccnc34)c2O1)N1CCNCC1 ; O=C([C@@H]1Cc2cc(Cl)cc(-c3ccnc4cccnc34)c2O1)N1CCNCC1]

### (R)-(5-chloro-7-(1,5-naphthyridin-4-yl)-2,3-dihydrobenzofuran-2-yl)(piperazin-1-yl)methanone and (S)-(5-chloro-7-(1,5-naphthyridin-4-yl)-2,3-dihydrobenzofuran-2-yl)(piperazin-1-yl)methanone

The example was prepared using precursor I, general procedures B and D as the HCl salt with 4-chloro-1,5-napthyridine. ¹H NMR (400 MHz, CD₃OD, hydrochloric acid salt): δ ppm 9.24 (d, J=4.9 Hz, 1H), 9.20 (dd, J=4.5, 1.6 Hz, 1H), 8.84 (dd, J=8.7, 1.5 Hz, 1H), 8.26 (d, J=4.9 Hz, 1H), 8.12 (dd, J=8.7, 4.6 Hz, 1H), 7.61 (d, J=2.2 Hz, 1H), 7.51-7.46 (m, 1H), 5.84 (dd, J=9.3, 6.4 Hz, 1H), 4.06-3.94 (m, 1H), 3.87-3.72 (m, 3H), 3.71-3.65 (m, 2H), 3.28-3.19 (m, J=17.4 Hz, 3H), 3.11-3.02 (m, 1H). LCMS: [M+H⁺]=395.0.

### Example 40

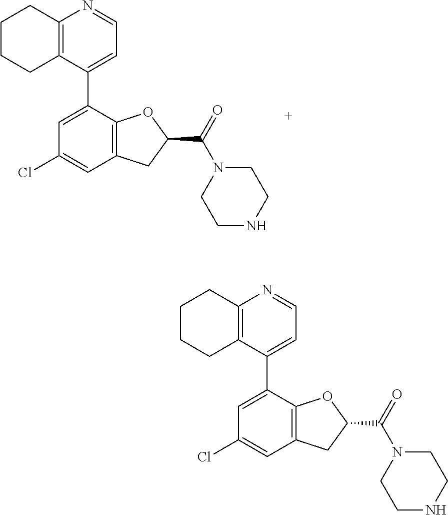 [SMILES: O=C([C@H]1Cc2cc(Cl)cc(-c3ccnc4c3CCCC4)c2O1)N1CCNCC1 ; O=C([C@@H]1Cc2cc(Cl)cc(-c3ccnc4c3CCCC4)c2O1)N1CCNCC1]

### (R)-(5-chloro-7-(5,6,7,8-tetrahydroquinolin-4-yl)-2,3-dihydrobenzofuran-2-yl)(piperazin-1-yl)methanone and (S)-(5-chloro-7-(5,6,7,8-tetrahydroquinolin-4-yl)-2,3-dihydrobenzofuran-2-yl)(piperazin-1-yl)methanone

The example was prepared using precursor I, general procedures B and D as the HCl salt with 4-chloro-5,6,7,8-tetrahydroquinoline. ¹H NMR (400 MHz, CD₃OD, hydrochloric acid salt): δ ppm 8.55 (d, J=5.8 Hz, 1H), 7.79 (d, J=6.2 Hz, 1H), 7.42 (s, 1H), 7.23 (s, 1H), 5.81 (dd, J=10.2, 6.1 Hz, 1H), 3.90-3.77 (m, 4H), 3.75-3.61 (m, 1H), 3.59-3.51 (m, 1H), 3.29-3.20 (m, 4H), 3.17 (t, J=6.4 Hz, 2H), 3.02-2.91 (m, 1H), 2.83-2.72 (m, 1H), 2.06-1.96 (m, 2H), 1.86 (s, 2H). LCMS: [M+H⁺]=398.0.

### Example 41

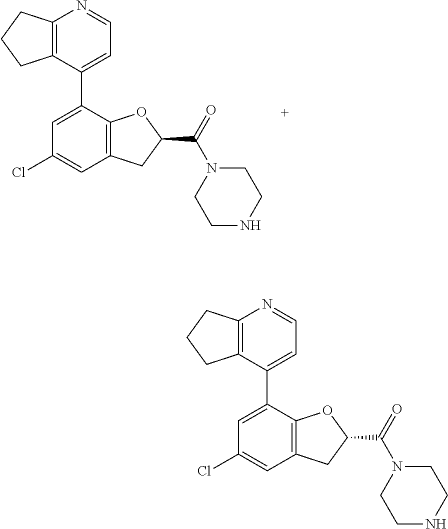 [SMILES: O=C([C@H]1Cc2cc(Cl)cc(-c3ccnc4c3CCC4)c2O1)N1CCNCC1 ; O=C([C@@H]1Cc2cc(Cl)cc(-c3ccnc4c3CCC4)c2O1)N1CCNCC1]

### (R)-(5-chloro-7-(6,7-dihydro-5H-cyclopenta[b]pyridin-4-yl)-2,3-dihydrobenzofuran-2-yl)(piperazin-1-yl)methanone and (S)-(5-chloro-7-(6,7-dihydro-5H-cyclopenta[b]pyridin-4-yl)-2,3-dihydrobenzofuran-2-yl)(piperazin-1-yl)methanone

The example was prepared using precursor I, general procedures B and D as the HCl salt with 4-chloro-6,7-dihydro-5H-cyclopenta[b]pyridine. ¹H NMR (400 MHz, CD₃OD, hydrochloric acid salt): δ ppm 8.53 (s, 1H), 7.94 (s, 1H), 7.47-7.42 (m, 1H), 7.37 (d, J=2.2 Hz, 1H), 5.86 (dd, J=10.1, 6.6 Hz, 1H), 3.92-3.82 (m, 3H), 3.72-3.53 (m, 2H), 3.35-3.19 (m, J=13.1 Hz, 8H), 3.19-3.10 (m, 1H), 2.34 (dd, J=15.0, 7.5 Hz, 2H). LCMS: [M+H^{+]}=384.1.

### Example 42

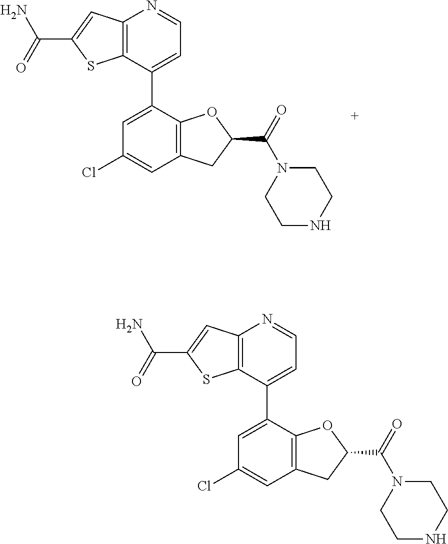 [SMILES: NC(=O)c1cc2nccc(-c3cc(Cl)cc4c3O[C@@H](C(=O)N3CCNCC3)C4)c2s1 ; NC(=O)c1cc2nccc(-c3cc(Cl)cc4c3O[C@H](C(=O)N3CCNCC3)C4)c2s1]

### (R)-7-(5-chloro-2-(piperazine-1-carbonyl)-2,3-dihydrobenzofuran-7-yl)thieno[3,2-b]pyridine-2-carboxamide and (S)-7-(5-chloro-2-(piperazine-1-carbonyl)-2,3-dihydrobenzofuran-7-yl)thieno[3,2-b]pyridine-2-carboxamide

The example was prepared using precursor I, general procedures B and D as the HCl salt with 7-chlorothieno-3,2-b]pyridine-2-carboxamide. ¹H NMR (400 MHz, CD₃OD, hydrochloric acid salt): δ ppm 8.78 (d, J=4.9 Hz, 1H), 8.17 (s, 1H), 7.64 (d, J=4.9 Hz, 1H), 7.49-7.45 (m, 2H), 5.81 (dd, J=9.9, 6.6 Hz, 1H), 4.03-3.93 (m, 1H), 3.92-3.73 (m, 4H), 3.58-3.46 (m, 2H), 3.25-3.21 (m, 2H), 2.98-.89 (m, 1H), LCMS: [M+H⁺]=443.0.

### Example 43

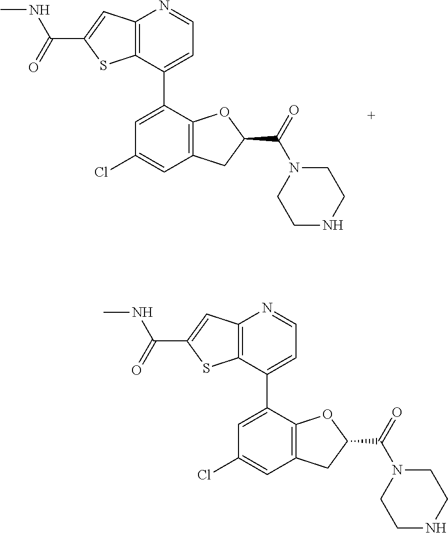 [SMILES: CNC(=O)c1cc2nccc(-c3cc(Cl)cc4c3O[C@@H](C(=O)N3CCNCC3)C4)c2s1 ; CNC(=O)c1cc2nccc(-c3cc(Cl)cc4c3O[C@H](C(=O)N3CCNCC3)C4)c2s1]

### (R)-7-(5-chloro-2-(piperazine-1-carbonyl)-2,3-dihydrobenzofuran-7-yl)thieno[3,2-b]pyridine-2-carboxamide and (S)-7-(5-chloro-2-(piperazine-1-carbonyl)-2,3-dihydrobenzofuran-7-yl)thieno[3,2-b]pyridine-2-carboxamide

The example was prepared using precursor I, general procedures B and D as the HCl salt with 7-(5-chloro-2-(piperazine-1-carbonyl)-2,3-dihydrobenzofuran-7-yl)-N-methylthieno[3,2-b]pyridine-2-carboxamide. ¹H NMR (400 MHz, CD₃OD, hydrochloric acid salt): δ ppm 9.02 (s, 1H), 8.28 (s, 1H), 8.16 (d, J=5.8 Hz, 1H), 7.66 (d, J=2.1 Hz, 1H), 7.60-7.54 (m, 1H), 5.92 (dd, J=10.0, 6.4 Hz, 1H), 4.01-3.81 (m, 3H), 3.81-3.71 (m, 1H), 3.70-3.56 (m, 2H), 3.26 (t, J=5.3 Hz, 3H), 3.19-3.07 (m, J=6.5 Hz, 1H), 3.00 (s, 3H). LCMS: [M+H⁺]=457.0.

### Example 44

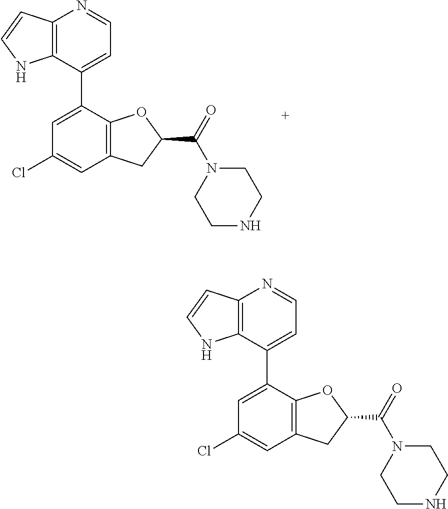 [SMILES: O=C([C@H]1Cc2cc(Cl)cc(-c3ccnc4cc[nH]c34)c2O1)N1CCNCC1 ; O=C([C@@H]1Cc2cc(Cl)cc(-c3ccnc4cc[nH]c34)c2O1)N1CCNCC1]

### (R)-(5-chloro-7-(1H-pyrrolo[3,2-b]pyridin-7-yl)-2,3-dihydrobenzofuran-2-yl)(piperazin-1-yl)methanone and (S)-(5-chloro-7-(1H-pyrrolo[3,2-b]pyridin-7-yl)-2,3-dihydrobenzofuran-2-yl)(piperazin-1-yl)methanone

The example was prepared using precursor I, general procedures B and D as the HCl salt with 7-chloro-1H-pyrrolo[3,2-b]pyridine. ¹H NMR (400 MHz, CD₃OD, hydrochloric acid salt): δ ppm 8.61 (d, J=6.3 Hz, 1H), 8.12 (d, J=3.2 Hz, 1H), 7.81 (d, J=6.1 Hz, 1H), 7.63 (d, J=2.2 Hz, 1H), 7.54 (s, 1H), 6.92 (d, J=3.2 Hz, 1H), 5.99 (dd, J=10.8, 3.9 Hz, 1H), 4.17-4.07 (m, 1H), 3.96-3.76 (m, 4H), 3.51 (dd, J=6.6, 3.7 Hz, 1H), 3.44-3.25 (m, 4H). LCMS: [M+H^{+]}=383.0.

### Example 45

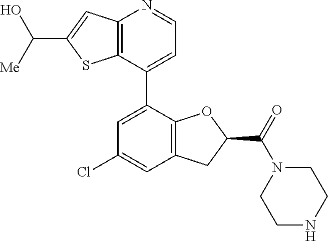 [SMILES: CC(O)c1cc2nccc(-c3cc(Cl)cc4c3O[C@@H](C(=O)N3CCNCC3)C4)c2s1]

### ((2R)-5-chloro-7-(2-(1-hydroxyethyl)thieno[3,2-b]pyridin-7-yl)-2,3-dihydrobenzofuran-2-yl)(piperazin-1-yl)methanone

The example was prepared using precursor II, general procedures B and D as the HCl salt with 1-(7-chlorothieno[3,2-b]pyridin-2-yl)ethan-1-ol. ¹H NMR (400 MHz, CD₃OD, hydrochloric acid salt): δ ppm 8.81 (br m, 1H), 7.91 (s, 1H), 7.65-7.54 (m, 2H), 7.53-7.43 (m, 1H), 5.91-5.78 (m, J=10.0, 6.7, 2.1 Hz, 1H), 5.35-5.23 (m, J=6.5 Hz, 1H), 4.04-3.68 (m, 6H), 3.68-3.54 (m, 1H), 3.29-3.16 (m, J=4.3 Hz, 2H), 3.13-2.98 (m, 1H), 1.70-1.61 (m, 3H). LCMS: [M+H⁺]=444.0.

### Example 46

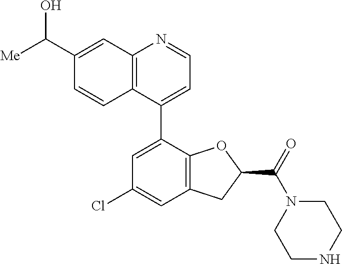 [SMILES: CC(O)c1ccc2c(-c3cc(Cl)cc4c3O[C@@H](C(=O)N3CCNCC3)C4)ccnc2c1]

### ((2R)-5-chloro-7-(7-(1-hydroxyethyl)quinolin-4-yl)-2,3-dihydrobenzofuran-2-yl)(piperazin-1-yl)methanone

The example was prepared using precursor II, general procedures B and D as the HCl salt with 1-(4-chloroquinolin-7-yl)ethan-1-ol. ¹H NMR (400 MHz, CD₃OD, hydrochloric acid salt): δ ppm 9.20 (s, 1H), 8.32 (d, J=11.5 Hz, 1H), 8.20 (dd, J=8.9, 5.6 Hz, 1H), 8.01-7.92 (m, 2H), 7.63 (s, 1H), 7.51 (d, J=1.5 Hz, 1H), 5.92-5.80 (m, 1H), 5.25 (q, J=6.7 Hz, 1H), 4.12-3.95 (m, 1H), 3.95-3.66 (m, 5H), 3.25 (d, J=1.6 Hz, 3H), 2.96-2.73 (m, 1H), 1.75-1.68 (m, 3H). LCMS: [M+H⁺]=438.0.

### Example 7

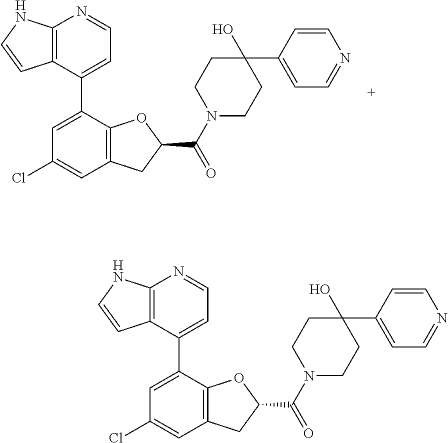 [SMILES: O=C([C@H]1Cc2cc(Cl)cc(-c3ccnc4[nH]ccc34)c2O1)N1CCC(O)(c2ccncc2)CC1 ; O=C([C@@H]1Cc2cc(Cl)cc(-c3ccnc4[nH]ccc34)c2O1)N1CCC(O)(c2ccncc2)CC1]

### (R)-(5-chloro-7-(1H-pyrrolo[2,3-b]pyridin-4-yl)-2,3-dihydrobenzofuran-2-yl)(4-hydroxy-4-(pyridin-4-yl)piperidin-1-yl)methanone and (S)-(5-chloro-7-(1H-pyrrolo[2,3-b]pyridin-4-yl)-2,3-dihydrobenzofuran-2-yl)(4-hydroxy-4-(pyridin-4-yl)piperidin-1-yl)methanone

The example was prepared using precursor I (step 5), general procedures A and C. The final product was converted to HCl salt using 4M HCl in dioxane. ¹H NMR (400 MHz, CD₃OD, hydrochloric acid salt): δ 8.84 (dd, J=12.2, 6.3 Hz, 2H), 8.43 (dd, J=14.7, 6.2 Hz, 1H), 8.30 (d, J=6.2 Hz, 1H), 8.15 (d, J=6.7 Hz, 1H), 7.87 (t, J=6.4 Hz, 1H), 7.74 (dd, J=9.4, 3.6 Hz, 1H), 7.57 (s, 1H), 7.49 (d, J=6.7 Hz, 1H), 7.10 (dd, J=26.0, 3.4 Hz, 1H), 5.94 (m, 1H), 4.55 (m, 1H), 4.00 (m, 1H), 3.80-3.52 (m, 4H), 3.21 (t, J=12.9 Hz, 1H), 2.08 (dd, J=65.7, 31.6 Hz, 2H), 1.78 (t, J=25.1 Hz, 1H). [M+H] 475.0.

### Example 48

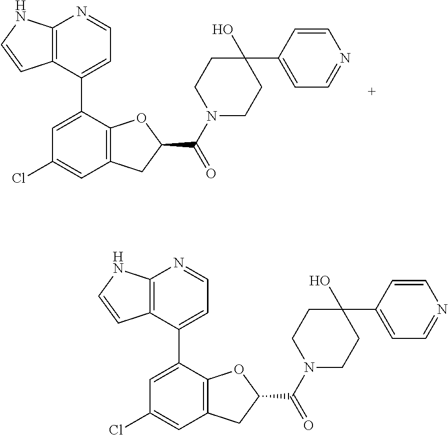 [SMILES: O=C([C@H]1Cc2cc(Cl)cc(-c3ccnc4[nH]ccc34)c2O1)N1CCC(O)(c2ccncc2)CC1 ; O=C([C@@H]1Cc2cc(Cl)cc(-c3ccnc4[nH]ccc34)c2O1)N1CCC(O)(c2ccncc2)CC1]

### (R)-(5-chloro-7-(1H-pyrrolo[2,3-b]pyridin-4-yl)-2,3-dihydrobenzofuran-2-yl)(4-hydroxy-4-(pyridin-3-yl)piperidin-1-yl)methanone and (S)-(5-chloro-7-(1H-pyrrolo[2,3-b]pyridin-4-yl)-2,3-dihydrobenzofuran-2-yl)(4-hydroxy-4-(pyridin-3-yl)piperidin-1-yl)methanone

The example was prepared using precursor I (step 5), general procedures A and C. The final product was converted to HCl salt using 4M HCl in dioxane. ¹H NMR (400 MHz, CD₃OD): δ 9.00 (d, J=27.7 Hz, 1H), 8.81 (d, J=5.8 Hz, 1H), 8.86-8.63 (m, 1H), 8.43 (dd, J=13.5, 6.2 Hz, 1H), 8.18-8.05 (m, 1H), 7.89 (dd, J=12.0, 6.3 Hz, 1H), 7.75 (dd, J=7.1, 3.7 Hz, 1H), 7.58 (d, J=2.2 Hz, 1H), 7.51-7.47 (m, 1H), 7.11 (dd, J=0.0, 3.7 Hz, 1H), 5.95 (M, 1H), 4.59-4.49 (m, 1H), 4.05-3.94 (m, 1H), 3.83-3.53 (m, 4H), 3.23 (t, J=12.8 Hz, 1H), 2.29-1.92 (m, 1H), 1.91-1.71 (m, 1H). [M+H] 475.0.

### Example 49

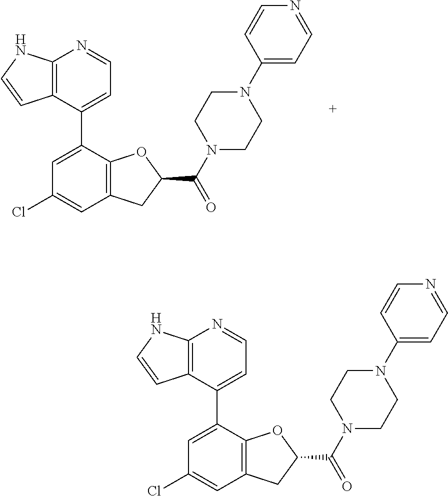 [SMILES: O=C([C@H]1Cc2cc(Cl)cc(-c3ccnc4[nH]ccc34)c2O1)N1CCN(c2ccncc2)CC1 ; O=C([C@@H]1Cc2cc(Cl)cc(-c3ccnc4[nH]ccc34)c2O1)N1CCN(c2ccncc2)CC1]

### (R)-(5-chloro-7-(1H-pyrrolo[2,3-b]pyridin-4-yl)-2,3-dihydrobenzofuran-2-yl)(4-(pyridin-4-yl)piperazin-1-yl)methanone and (S)-(5-chloro-7-(1H-pyrrolo[2,3-b]pyridin-4-yl)-2,3-dihydrobenzofuran-2-yl)(4-(pyridin-4-yl)piperazin-1-yl)methanone

The example was prepared using precursor I (step 5), general procedures A and C. The final product was converted to HCl salt using 4M HCl in dioxane. ¹H NMR (400 MHz, CD₃OD, hydrochloric acid salt): δ 8.44 (d, J=6.2 Hz, 1H), 8.20 (d, J=7.6 Hz, 2H), 7.83 (d, J=6.2 Hz, 1H), 7.73 (d, J=3.6 Hz, 1H), 7.57 (d, J=2.1 Hz, 1H), 7.48 (d, J=2.1 Hz, 1H), 7.18 (d, J=7.7 Hz, 2H), 7.06 (d, J=3.6 Hz, 1H), 5.90 (dd, J=9.8, 6.7 Hz, 1H), 3.97-3.64 (m, 10H). [M+H] 460.0.

### Example 50

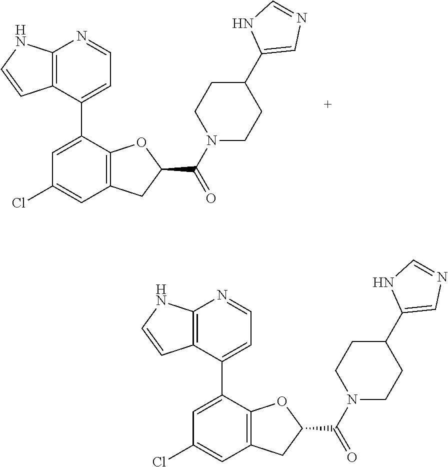 [SMILES: O=C([C@H]1Cc2cc(Cl)cc(-c3ccnc4[nH]ccc34)c2O1)N1CCC(c2cnc[nH]2)CC1 ; O=C([C@@H]1Cc2cc(Cl)cc(-c3ccnc4[nH]ccc34)c2O1)N1CCC(c2cnc[nH]2)CC1]

### (R)-(4-(1H-imidazol-5-yl)piperidin-1-yl)(5-chloro-7-(1H-pyrrolo[2,3-b]pyridin-4-yl)-2,3-dihydrobenzofuran-2-yl)methanone and (S)-(4-(1H-imidazol-5-yl)piperidin-1-yl)(5-chloro-7-(1H-pyrrolo[2,3-b]pyridin-4-yl)-2,3-dihydrobenzofuran-2-yl)methanone

The example was prepared using precursor I (step 5), general procedures A and C. ¹H NMR (400 MHz, CD₃OD, free base): δ 8.18 (dd, J=34.8, 5.2 Hz, 1H), 7.67 (s, 1H), 7.46-7.25 (m, 4H), 6.88-6.50 (m, 2H), 5.80-5.71 (m, 1H), 4.58-4.44 (m, 1H), 4.21-4.02 (m, 1H), 3.82-3.42 (m, 2H), 2.97-2.74 (m, 2H), 1.99 (d, J=12.0 Hz, 1H), 1.79-1.31 (m, 4H). [M+H] 448.0.

### Example 51

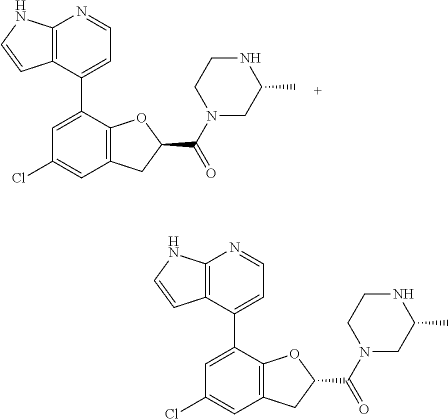 [SMILES: C[C@@H]1CN(C(=O)[C@H]2Cc3cc(Cl)cc(-c4ccnc5[nH]ccc45)c3O2)CCN1 ; C[C@@H]1CN(C(=O)[C@@H]2Cc3cc(Cl)cc(-c4ccnc5[nH]ccc45)c3O2)CCN1]

### ((R)-5-chloro-7-(1H-pyrrolo[2,3-b]pyridin-4-yl)-2,3-dihydrobenzofuran-2-yl)((R)-3-methylpiperazin-1-yl)methanone and ((S)-5-chloro-7-(1H-pyrrolo[2,3-b]pyridin-4-yl)-2,3-dihydrobenzofuran-2-yl)((R)-3-methylpiperazin-1-yl)methanone

The example was prepared using precursor I (step 5), general procedures A, C, and D. ¹H NMR (400 MHz, CD₃OD, hydrochloric acid salt): δ 8.49-8.44 (m, 1H), 7.84-7.73 (m, 2H), 7.59-7.52 (m, 1H), 7.49-7.45 (m, 1H), 7.08-6.97 (m, 1H), 5.96-5.82 (m, 1H), 4.64-4.43 (m, 1H), 4.25-4.10 (m, 1H), 3.80-3.59 (m, 2H), 3.57-3.33 (m, 2H), 3.26-2.88 (m, 3H), 1.34 (dd, J=6.5, 3.3 Hz, 2H), 1.26-1.16 (m, 1H). [M+H] 397.0.

### Example 52

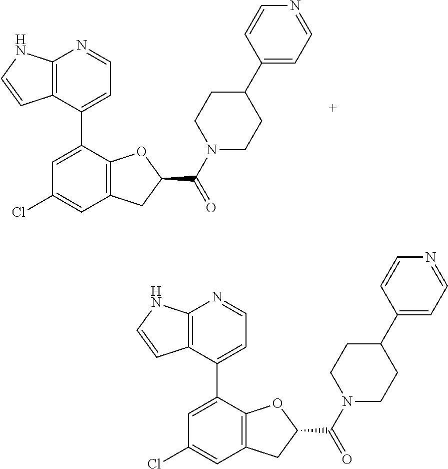 [SMILES: O=C([C@H]1Cc2cc(Cl)cc(-c3ccnc4[nH]ccc34)c2O1)N1CCC(c2ccncc2)CC1 ; O=C([C@@H]1Cc2cc(Cl)cc(-c3ccnc4[nH]ccc34)c2O1)N1CCC(c2ccncc2)CC1]

### (R)-(5-chloro-7-(1H-pyrrolo[2,3-b]pyridin-4-yl)-2,3-dihydrobenzofuran-2-yl)(4-(pyridin-4-yl)piperidin-1-yl)methanone and (S)-(5-chloro-7-(1H-pyrrolo[2,3-b]pyridin-4-yl)-2,3-dihydrobenzofuran-2-yl)(4-(pyridin-4-yl)piperidin-1-yl)methanone

The example was prepared using precursor I (step 5), general procedures A and C. The final product was converted to HCl salt using 4M HCl in dioxane. ¹H NMR (400 MHz, CD₃OD, hydrochloric acid salt): δ 8.79 (dd, J=9.8, 6.7 Hz, 2H), 8.41 (dd, J=17.9, 6.1 Hz, 1H), 8.07 (d, J=6.7 Hz, 1H), 7.91 (d, J=6.6 Hz, 1H), 7.83 (dd, J=11.6, 6.2 Hz, 1H), 7.72 (dd, J=17.5, 3.6 Hz, 1H), 7.56 (d, J=4.4 Hz, 1H), 7.48 (d, J=4.2 Hz, 1H), 7.08-7.04 (m, 1H), 5.95-5.87 (m, 1H), 4.72 (t, J=12.9 Hz, 1H), 4.20 (d, J=14.0 Hz, 1H), 3.80-3.51 (m, 3H), 2.88 (dd, J=20.1, 10.5 Hz, 1H), 2.15-1.51 (m, 3H). [M+H] 459.0.

### Example 53

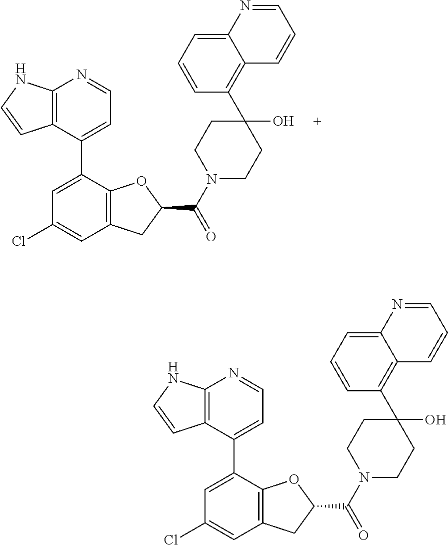 [SMILES: O=C([C@H]1Cc2cc(Cl)cc(-c3ccnc4[nH]ccc34)c2O1)N1CCC(O)(c2cccc3ncccc23)CC1 ; O=C([C@@H]1Cc2cc(Cl)cc(-c3ccnc4[nH]ccc34)c2O1)N1CCC(O)(c2cccc3ncccc23)CC1]

### (R)-(5-chloro-7-(1H-pyrrolo[2,3-b]pyridin-4-yl)-2,3-dihydrobenzofuran-2-yl)(4-hydroxy-4-(quinolin-5-yl)piperidin-1-yl)methanone and (S)-(5-chloro-7-(1H-pyrrolo[2,3-b]pyridin-4-yl)-2,3-dihydrobenzofuran-2-yl)(4-hydroxy-4-(quinolin-5-yl)piperidin-1-yl)methanone

The example was prepared using precursor I (step 5), general procedures A and C. The final product was converted to HCl salt using 4M HCl in dioxane (1 mL). ¹H NMR (400 MHz, CD₃OD, hydrochloric acid salt): δ 10.35 (dd, J=38.8, 9.2 Hz, 1H), 9.22-9.16 (m, J=6.3 Hz, 1H), 8.37 (dd, J=59.8, 6.0 Hz, 1H), 8.22-7.99 (m, 3H), 7.85 (d, J=6.2 Hz, 1H), 7.76-7.69 (m, 1H), 7.64 (d, J=3.4 Hz, 1H), 7.55 (dd, J=15.6, 2.1 Hz, 1H), 7.48-7.44 (m, 1H), 7.03 (dd, J=22.2, 3.6 Hz, 1H), 6.00-5.85 (m, J=9.3 Hz, 1H), 4.60-4.48 (m, 1H), 4.08-3.96 (m, 1H), 3.93-3.91 (m, 1H), 3.89-3.56 (m, 3H), 3.53-3.37 (m, 1H), 2.39-1.87 (m, 3H). [M+H] 525.0.

### Example 54

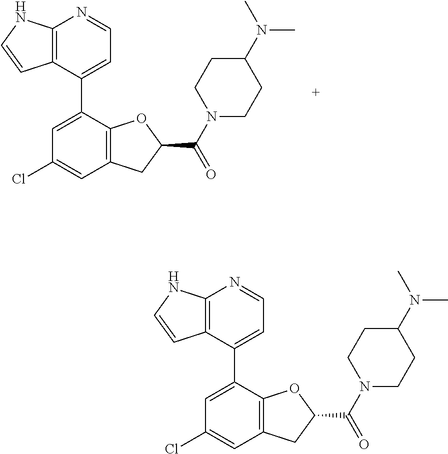 [SMILES: CN(C)C1CCN(C(=O)[C@H]2Cc3cc(Cl)cc(-c4ccnc5[nH]ccc45)c3O2)CC1 ; CN(C)C1CCN(C(=O)[C@@H]2Cc3cc(Cl)cc(-c4ccnc5[nH]ccc45)c3O2)CC1]

### (R)-(5-chloro-7-(1H-pyrrolo[2,3-b]pyridin-4-yl)-2,3-dihydrobenzofuran-2-yl)(4-(dimethylamino)piperidin-1-yl)methanone and (S)-(5-chloro-7-(1H-pyrrolo[2,3-b]pyridin-4-yl)-2,3-dihydrobenzofuran-2-yl)(4-(dimethylamino)piperidin-1-yl)methanone

The example was prepared using precursor I (step 5), general procedures A and C. The final product was converted to HCl salt using 4M HCl in dioxane (1 mL). ¹H NMR (400 MHz, CD₃OD, hydrochloric acid salt): δ 8.46-8.42 (m, 1H), 7.83 (t, J=6.6 Hz, 1H), 7.76-7.71 (m, 1H), 7.56 (s, 1H), 7.47 (d, J=5.0 Hz, 1H), 7.06 (dd, J=22.0, 3.4 Hz, 1H), 5.88 (dd, J=16.3, 9.9 Hz, 1H), 4.77-4.63 (m, 1H), 4.26-4.16 (m, 1H), 3.75-3.62 (m, J=0.8 Hz, 3H), 3.59-3.41 (m, J=19.8, 12.2 Hz, 2H), 3.19 (dd, J=25.0, 12.7 Hz, 1H), 2.90 (s, 3H), 2.80 (d, J=5.1 Hz, 3H), 2.22-2.01 (m, 2H). [M+H] 425.0.

### Example 55

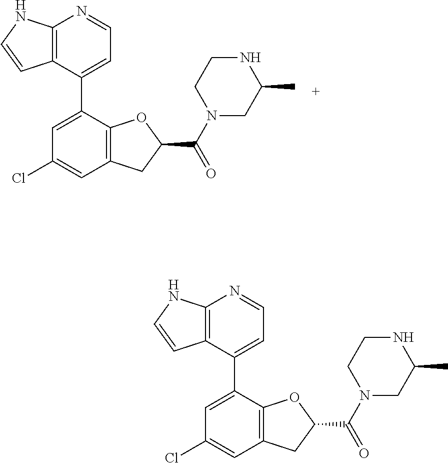 [SMILES: C[C@H]1CN(C(=O)[C@H]2Cc3cc(Cl)cc(-c4ccnc5[nH]ccc45)c3O2)CCN1 ; C[C@H]1CN(C(=O)[C@@H]2Cc3cc(Cl)cc(-c4ccnc5[nH]ccc45)c3O2)CCN1]

### ((R)-5-chloro-7-(1H-pyrrolo[2,3-b]pyridin-4-yl)-2,3-dihydrobenzofuran-2-yl)((S)-3-methylpiperazin-1-yl)methanone and ((S)-5-chloro-7-(1H-pyrrolo[2,3-b]pyridin-4-yl)-2,3-dihydrobenzofuran-2-yl)((S)-3-methylpiperazin-1-yl)methanone

The example was prepared using precursor I (step 5), general procedures A, C, and D. ¹H NMR (400 MHz, CD₃OD, hydrochloric acid salt): δ 8.50-8.43 (m, 1H), 7.84-7.74 (m, 2H), 7.60-7.53 (m, 1H), 7.48 (dd, J=3.6, 2.5 Hz, 1H), 7.08-6.96 (m, 1H), 5.90 (dd, J=9.9, 6.7 Hz, 1H), 4.62-4.44 (m, 1H), 4.25-4.10 (m, 1H), 3.83-3.67 (m, 2H), 3.58-3.34 (m, 2H), 3.26-2.83 (m, 3H), 1.35 (dd, J=6.5, 3.2 Hz, 2H), 1.21 (dd, J=7.4, 2.2 Hz, 1H). [M+H] 397.0.

### Example 56

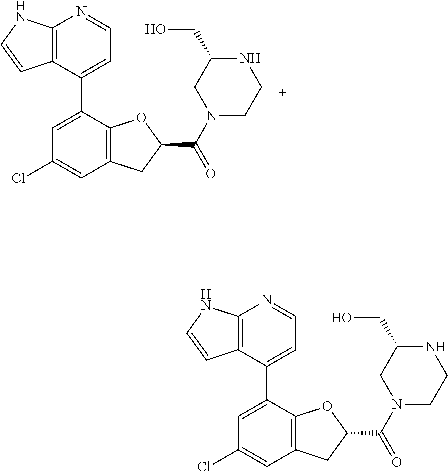 [SMILES: O=C([C@H]1Cc2cc(Cl)cc(-c3ccnc4[nH]ccc34)c2O1)N1CCN[C@@H](CO)C1 ; O=C([C@@H]1Cc2cc(Cl)cc(-c3ccnc4[nH]ccc34)c2O1)N1CCN[C@@H](CO)C1]

### ((R)-5-chloro-7-(1H-pyrrolo[2,3-b]pyridin-4-yl)-2,3-dihydrobenzofuran-2-yl)(R)-3-(hydroxymethyl)piperazin-1-yl)methanone and ((S)-5-chloro-7-(1H-pyrrolo[2,3-b]pyridin-4-yl)-2,3-dihydrobenzofuran-2-yl)((R)-3-(hydroxymethyl)piperazin-1-yl)methanone

The example was prepared using precursor I (step 5), general procedures A, C, and D. The final compound was dissolved in CH₂Cl₂, washed with saturated NaHCO₃ solution, and concentrated in vacuo to afford the free base. ¹H NMR (400 MHz, CD₃OD, free base): δ 8.23 (dd, J=7.2, 3.7 Hz, 1H), 7.49-7.23 (m, 4H), 6.59-6.51 (m, 1H), 5.79-5.68 (m, 1H), 4.69-3.41 (m, 5H), 3.17-2.29 (m, 4H), 1.34-1.18 (m, 2H). [M+H] 413.0.

### Example 57

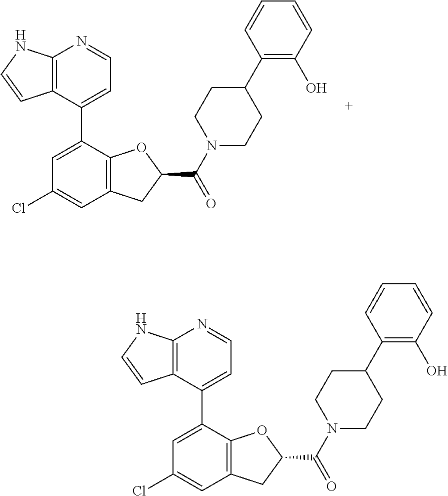 [SMILES: O=C([C@H]1Cc2cc(Cl)cc(-c3ccnc4[nH]ccc34)c2O1)N1CCC(c2ccccc2O)CC1 ; O=C([C@@H]1Cc2cc(Cl)cc(-c3ccnc4[nH]ccc34)c2O1)N1CCC(c2ccccc2O)CC1]

### (R)-(5-chloro-7-(1H-pyrrolo[2,3-b]pyridin-4-yl)-2,3-dihydrobenzofuran-2-yl)(4-(2-hydroxyphenyl)piperidin-1-yl)methanone and (S)-(5-chloro-7-(1H-pyrrolo[2,3-b]pyridin-4-yl)-2,3-dihydrobenzofuran-2-yl)(4-(2-hydroxyphenyl)piperidin-1-yl)methanone

The example was prepared using precursor I (step 5), general procedures A and C. The final product was converted to HCl salt using 4M HCl in dioxane. ¹H NMR (400 MHz, CD₃OD, hydrochloric acid salt) δ 8.40 (dd, J=26.2, 6.2 Hz, 1H), 7.84 (t, J=6.3 Hz, 1H), 7.74 (dd, J=5.8, 3.6 Hz, 1H), 7.56 (s, 1H), 7.50 (s, 1H), 7.06 (td, J=19.2, 11.6 Hz, 2H), 6.74 (ddd, J=16.5, 13.2, 6.4 Hz, 3H), 5.93-5.86 (m, 1H), 4.71-4.56 (m, 1H), 4.13 (t, J=13.8 Hz, 1H), 3.79-3.56 (m, 3H), 3.27-3.11 (m, 2H), 2.89-2.75 (m, 1H), 1.99-1.26 (m, 3H). [M+H] 474.0.

### Example 58

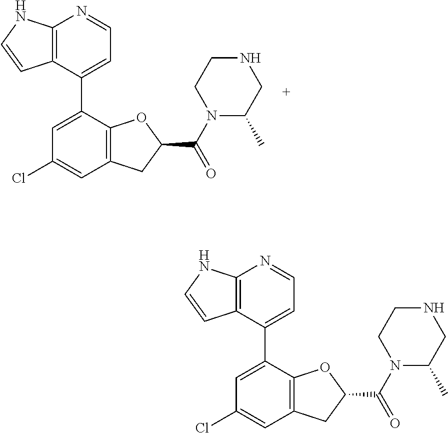 [SMILES: C[C@H]1CNCCN1C(=O)[C@H]1Cc2cc(Cl)cc(-c3ccnc4[nH]ccc34)c2O1 ; C[C@H]1CNCCN1C(=O)[C@@H]1Cc2cc(Cl)cc(-c3ccnc4[nH]ccc34)c2O1]

### ((R)-5-chloro-7-(1H-pyrrolo[2,3-b]pyridin-4-yl)-2,3-dihydrobenzofuran-2-yl)((S)-2-methylpiperazin-1-yl)methanone and ((S)-5-chloro-7-(1H-pyrrolo[2,3-b]pyridin-4-yl)-2,3-dihydrobenzofuran-2-yl)((S)-2-methylpiperazin-1-yl)methanone

The example was prepared using precursor I (step 5), general procedures A, C, and D. ¹H NMR (400 MHz, CD₃OD, hydrochloric acid salt): δ 8.44 (d, J=6.1 Hz, 1H), 7.80 (d, J=6.2 Hz, 1H), 7.74 (d, J=3.6 Hz, 1H), 7.56 (d, J=2.1 Hz, 1H), 7.48 (s, 1H), 7.04 (d, J=22.6 Hz, 1H), 5.92-5.77 (m, 1H), 4.65-4.41 (m, 1H), 4.17-3.99 (m, 1H), 3.66 (s, 3H), 3.60-3.43 (m, 2H), 3.24-2.87 (m, 2H), 1.61-1.12 (m, 3H). [M+H] 397.0.

### Example 59

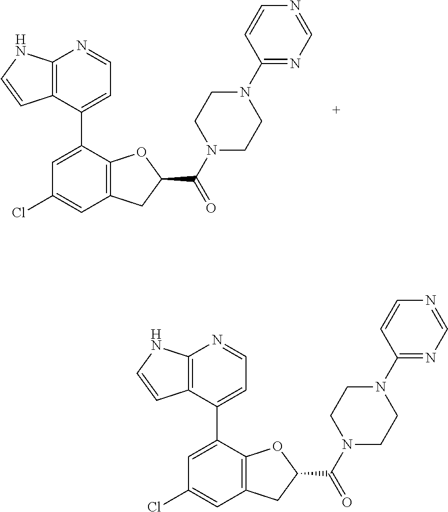 [SMILES: O=C([C@H]1Cc2cc(Cl)cc(-c3ccnc4[nH]ccc34)c2O1)N1CCN(c2ccncn2)CC1 ; O=C([C@@H]1Cc2cc(Cl)cc(-c3ccnc4[nH]ccc34)c2O1)N1CCN(c2ccncn2)CC1]

### (R)-(5-chloro-7-(1H-pyrrolo[2,3-b]pyridin-4-yl)-2,3-dihydrobenzofuran-2-yl)(4-(pyrimidin-4-yl)piperazin-1-yl)methanone and (S)-(5-chloro-7-(1H-pyrrolo[2,3-b]pyridin-4-yl)-2,3-dihydrobenzofuran-2-yl)(4-(pyrimidin-4-yl)piperazin-1-yl)methanone

The example was prepared using precursor I (step 5), general procedures A and C. The final product was converted to HCl salt using 4M HCl in dioxane. ¹H NMR (400 MHz, CD₃OD, hydrochloric acid salt): δ 8.74 (s, 1H), 8.44 (d, J=6.2 Hz, 1H), 8.25 (dd, J=7.6, 1.5 Hz, 1H), 7.83 (d, J=6.2 Hz, 1H), 7.74 (d, J=3.6 Hz, 1H), 7.57 (d, J=2.1 Hz, 1H), 7.48 (d, J=2.0 Hz, 1H), 7.14 (d, J=25.9 Hz, 1H), 7.05 (d, J=3.6 Hz, 1H), 5.91 (dd, J=9.5, 7.1 Hz, 1H), 4.36-3.70 (m, 8H), 3.71-3.63 (m, 2H). [M+H] 461.1.

### Example 60

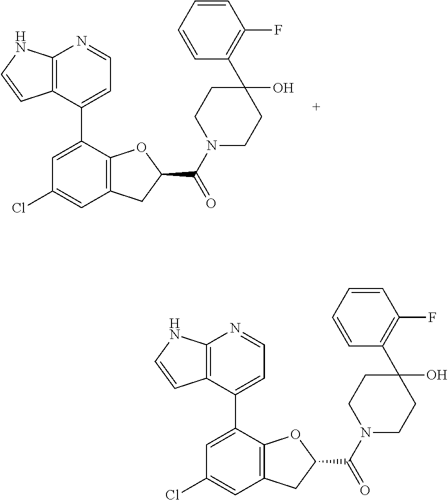 [SMILES: O=C([C@H]1Cc2cc(Cl)cc(-c3ccnc4[nH]ccc34)c2O1)N1CCC(O)(c2ccccc2F)CC1 ; O=C([C@@H]1Cc2cc(Cl)cc(-c3ccnc4[nH]ccc34)c2O1)N1CCC(O)(c2ccccc2F)CC1]

### (R)-(5-chloro-7-(1H-pyrrolo[2,3-b]pyridin-4-yl)-2,3-dihydrobenzofuran-2-yl)(4-(2-fluorophenyl)-4-hydroxypiperidin-1-yl)methanone and (S)-(5-chloro-7-(1H-pyrrolo[2,3-b]pyridin-4-yl)-2,3-dihydrobenzofuran-2-yl)(4-(2-fluorophenyl)-4-hydroxypiperidin-1-yl)methanone

The example was prepared using precursor I (step 5), general procedures A and C. The final product was converted to HCl salt using 4M HCl in dioxane. ¹H NMR (400 MHz, CD₃OD, hydrochloric acid salt): δ 8.39 (dd, J=29.4, 6.1 Hz, 1H), 7.83 (t, J=6.6 Hz, 1H), 7.75-7.70 (m, 1H), 7.69-7.52 (m, 2H), 7.52-7.49 (m, 1H), 7.33-7.24 (m, 1H), 7.17 (dtd, J=8.7, 7.5, 1.2 Hz, 1H), 7.04 (d, J=3.5 Hz, 1H), 6.92-6.81 (m, 1H), 5.98-5.88 (m, 1H), 4.42 (dd, J=26.4, 13.2 Hz, 1H), 3.95 (dd, J=26.3, 11.3 Hz, 1H), 3.85-3.71 (m, 1H), 3.65-3.54 (m, 2H), 3.27-3.10 (m, 1H), 2.42-1.37 (m, 3H). [M+H] 465.0.

### Example 61

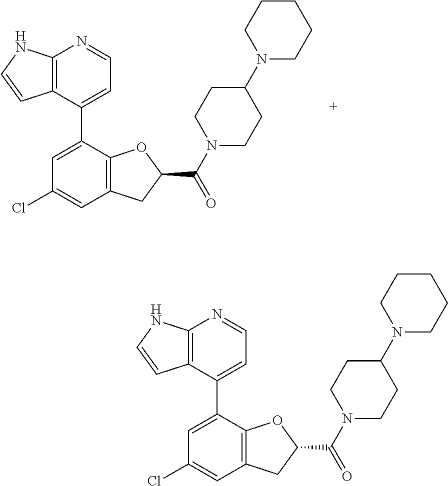 [SMILES: O=C([C@H]1Cc2cc(Cl)cc(-c3ccnc4[nH]ccc34)c2O1)N1CCC(N2CCCCC2)CC1 ; O=C([C@@H]1Cc2cc(Cl)cc(-c3ccnc4[nH]ccc34)c2O1)N1CCC(N2CCCCC2)CC1]

### (R)-[1,4′-bipiperidin]-1′-yl(5-chloro-7-(1H-pyrrolo[2,3-b]pyridin-4-yl)-2,3-dihydrobenzofuran-2-yl)methanone and (S)-[1,4′-bipiperidin]-1′-yl(5-chloro-7-(1H-pyrrolo[2,3-b]pyridin-4-yl)-2,3-dihydrobenzofuran-2-yl)methanone

The example was prepared using precursor I (step 5), general procedures A and C. The final product was converted to HCl salt using 4M HCl in dioxane. ¹H NMR (400 MHz, CD₃OD, hydrochloric acid salt): δ 8.46 (dd, J=12.2, 6.3 Hz, 1H), 7.87 (dd, J=17.0, 6.3 Hz, 1H), 7.79-7.74 (m, 1H), 7.57 (t, J=2.1 Hz, 1H), 7.50-7.45 (m, 1H), 7.09 (dd, J=29.6, 3.6 Hz, 1H), 5.94-5.84 (m, 1H), 4.69 (t, J=11.2 Hz, 1H), 4.20 (d, J=13.6 Hz, 1H), 3.75-3.67 (m, 1H), 3.61-3.33 (m, 5H), 3.28-2.64 (m, 4H), 2.31-1.75 (m, 7H), 1.73-1.42 (m, 2H). [M+H] 465.0.

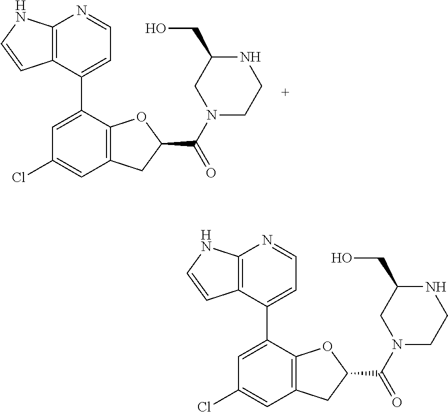 [SMILES: O=C([C@H]1Cc2cc(Cl)cc(-c3ccnc4[nH]ccc34)c2O1)N1CCN[C@H](CO)C1 ; O=C([C@@H]1Cc2cc(Cl)cc(-c3ccnc4[nH]ccc34)c2O1)N1CCN[C@H](CO)C1]

### Example 62

### ((R)-5-chloro-7-(1H-pyrrolo[2,3-b]pyridin-4-yl)-2,3-dihydrobenzofuran-2-yl)(S)-3-(hydroxymethyl)piperazin-1-yl)methanone and ((S)-5-chloro-7-(1H-pyrrolo[2,3-b]pyridin-4-yl)-2,3-dihydrobenzofuran-2-yl)((S)-3-(hydroxymethyl)piperazin-1-yl)methanone

The example was prepared using precursor I (step 5), general procedures A, C, and D. The final compound was dissolved in CH₂Cl₂, washed with saturated NaHCO₃ solution, and concentrated in vacuo to afford the free base. ¹H NMR (400 MHz, CD₃OD, free base): δ 8.33-8.19 (m, J=5.2 Hz, 1H), 7.48-7.19 (m, 4H), 6.64-6.46 (m, 1H), 5.80-5.66 (m, 1H), 4.52-3.40 (m, 4H), 3.15-2.07 (m, 4H), 1.29 (s, 2H), 0.97-0.80 (m, 1H). [M+H] 413.0.

### Example 63

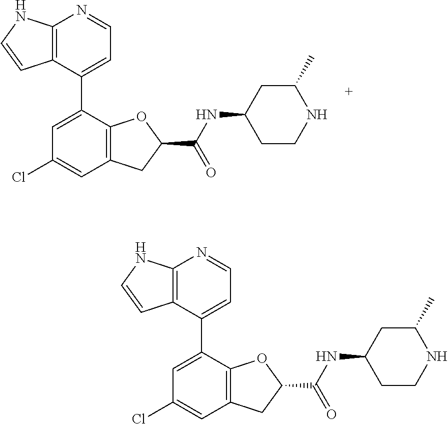 [SMILES: C[C@H]1C[C@H](NC(=O)[C@H]2Cc3cc(Cl)cc(-c4ccnc5[nH]ccc45)c3O2)CCN1 ; C[C@H]1C[C@H](NC(=O)[C@@H]2Cc3cc(Cl)cc(-c4ccnc5[nH]ccc45)c3O2)CCN1]

### (R)-5-chloro-N-((2S,4R)-2-methylpiperidin-4-yl)-7-(1H-pyrrolo[2,3-b]pyridin-4-yl)-2,3-dihydrobenzofuran-2-carboxamide and (S)-5-chloro-N-((2S,4R)-2-methylpiperidin-4-yl)-7-(1H-pyrrolo[2,3-b]pyridin-4-yl)-2,3-dihydrobenzofuran-2-carboxamide

The example was prepared using precursor I (step 5), general procedures A, C, and D. ¹H NMR (400 MHz, CD₃OD, hydrochloric acid salt): δ 8.51-8.41 (m, 2H), 7.82 (dd, J=6.3, 2.3 Hz, 1H), 7.75 (d, J=3.6 Hz, 1H), 7.55 (d, J=1.3 Hz, 1H), 7.47 (d, J=1.7 Hz, 1H), 7.06-6.99 (m, 1H), 5.41 (ddd, J=10.1, 6.7, 1.5 Hz, 1H), 4.29-4.15 (m, 1H), 3.79-3.67 (m, 1H), 3.60-3.37 (m, 2H), 3.26-3.18 (m, 1H), 2.12-1.92 (m, 3H), 1.80 (ddt, J=14.3, 10.2, 3.8 Hz, 1H), 1.35 (dd, J=6.6, 5.0 Hz, 3H), 1.20 (s, 1H). [M+H] 411.1.

### Example 64

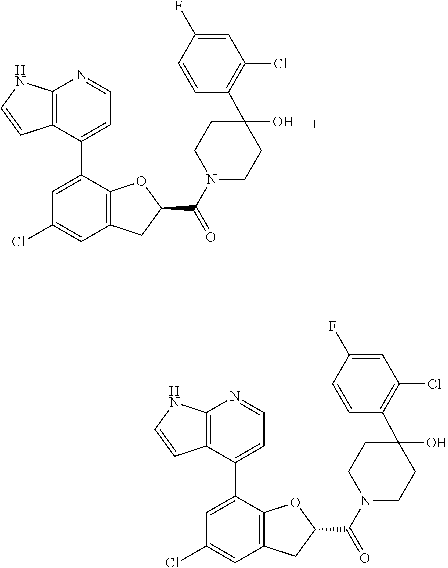 [SMILES: O=C([C@H]1Cc2cc(Cl)cc(-c3ccnc4[nH]ccc34)c2O1)N1CCC(O)(c2ccc(F)cc2Cl)CC1 ; O=C([C@@H]1Cc2cc(Cl)cc(-c3ccnc4[nH]ccc34)c2O1)N1CCC(O)(c2ccc(F)cc2Cl)CC1]

### (R)-(4-(2-chloro-4-fluorophenyl)-4-hydroxypiperidin-1-yl)(5-chloro-7-(1H-pyrrolo[2,3-b]pyridin-4-yl)-2,3-dihydrobenzofuran-2-yl)methanone and (S)-(4-(2-chloro-4-fluorophenyl)-4-hydroxypiperidin-1-yl)(5-chloro-7-(1H-pyrrolo[2,3-b]pyridin-4-yl)-2,3-dihydrobenzofuran-2-yl)methanone

The example was prepared using precursor I (step 5), general procedures A and C and purified by reverse phase HPLC using water (0.1% TFA) and acetonitrile (0.1% TFA) as the eluent. ¹H NMR (400 MHz, CD₃OD, trifluoroacetic acid salt): δ 8.34 (dd, J=24.4, 5.5 Hz, 1H), 7.69 (ddd, J=12.3, 6.3, 3.5 Hz, 2H), 7.64 (d, J=3.6 Hz, 1H), 7.52 (d, J=2.0 Hz, 1H), 7.45 (d, J=2.0 Hz, 1H), 7.14-6.95 (m, 2H), 6.92 (t, J=3.8 Hz, 1H), 5.90 (dt, J=15.6, 7.8 Hz, 1H), 4.41 (ddd, J=14.9, 12.9, 6.5 Hz, 1H), 4.11-3.88 (m, 1H), 3.84 (dd, J=16.3, 6.6 Hz, 1H), 3.64-3.49 (m, 2H), 3.27-3.08 (m, 1H), 2.65 (tdd, J=13.6, 8.8, 4.8 Hz, 1H), 2.46 (td, J=13.3, 4.9 Hz, 1H), 2.23 (td, J=13.3, 4.8 Hz, 1H), 1.79-1.63 (m, 1H), 1.50 (d, J=15.5 Hz, 1H). [M+H] 526.0.

### Example 65

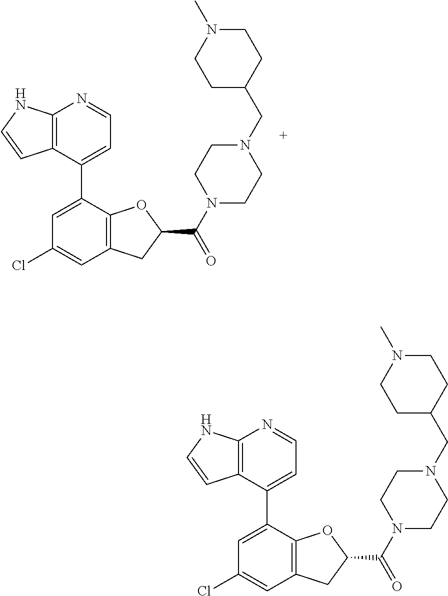 [SMILES: CN1CCC(CN2CCN(C(=O)[C@H]3Cc4cc(Cl)cc(-c5ccnc6[nH]ccc56)c4O3)CC2)CC1 ; CN1CCC(CN2CCN(C(=O)[C@@H]3Cc4cc(Cl)cc(-c5ccnc6[nH]ccc56)c4O3)CC2)CC1]

### (R)-(5-chloro-7-(1H-pyrrolo[2,3-b]pyridin-4-yl)-2,3-dihydrobenzofuran-2-yl)(4-((1-methylpiperidin-4-yl)methyl)piperazin-1-yl)methanone and (S)-(5-chloro-7-(1H-pyrrolo[2,3-b]pyridin-4-yl)-2,3-dihydrobenzofuran-2-yl)(4-((1-methylpiperidin-4-yl)methyl)piperazin-1-yl)methanone

The example was prepared using precursor I (step 5), general procedures A and C. The final product was converted to HCl salt using 4M HCl in dioxane. ¹H NMR (400 MHz, CD₃OD, hydrochloric acid salt): δ 8.50-8.45 (m, 1H), 7.80 (d, J=17.3 Hz, 2H), 7.57 (s, 1H), 7.49 (s, 1H), 7.02 (d, J=36.1 Hz, 1H), 5.88 (t, J=8.1 Hz, 1H), 4.62 (d, J=13.9 Hz, 1H), 4.24 (s, 1H), 3.64 (dd, J=50.9, 23.9 Hz, 9H), 3.21-3.01 (m, 6H), 2.89 (s, 3H), 2.20 (d, J=11.7 Hz, 3H), 1.71-1.52 (m, 1H). [M+H] 494.1.

### Example 66

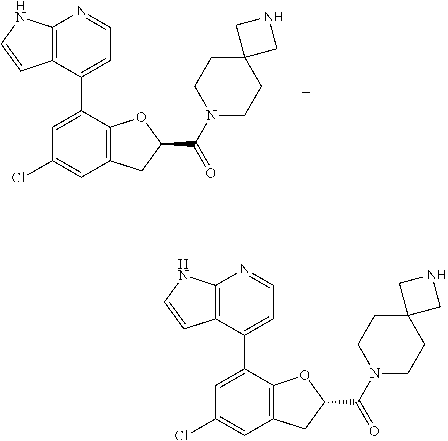 [SMILES: O=C([C@H]1Cc2cc(Cl)cc(-c3ccnc4[nH]ccc34)c2O1)N1CCC2(CC1)CNC2 ; O=C([C@@H]1Cc2cc(Cl)cc(-c3ccnc4[nH]ccc34)c2O1)N1CCC2(CC1)CNC2]

### (R)-(5-chloro-7-(1H-pyrrolo[2,3-b]pyridin-4-yl)-2,3-dihydrobenzofuran-2-yl)(2,7-diazaspiro[3.5]nonan-7-yl)methanone and (S)-(5-chloro-7-(1H-pyrrolo[2,3-b]pyridin-4-yl)-2,3-dihydrobenzofuran-2-yl)(2,7-diazaspiro[3.5]nonan-7-yl)methanone

The example was prepared using precursor I (step 5), general procedures A, C, and D. ¹H NMR (400 MHz, CD₃OD, hydrochloric acid salt): δ 8.45 (d, J=5.6 Hz, 1H), 7.86 (dd, J=6.2, 3.3 Hz, 1H), 7.75 (d, J=3.6 Hz, 1H), 7.56 (d, J=2.1 Hz, 1H), 7.47 (s, 1H), 7.08 (d, J=2.1 Hz, 1H), 5.96-5.79 (m, 1H), 4.20-3.79 (m, 3H), 3.61-3.49 (m, 3H), 2.95 (s, 1H), 2.21 (s, 1H), 2.12-1.66 (m, 4H), 1.42 (s, 2H). [M+H] 423.0.

### Example 67

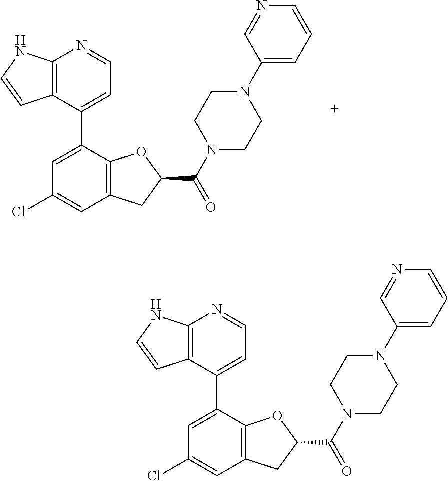 [SMILES: O=C([C@H]1Cc2cc(Cl)cc(-c3ccnc4[nH]ccc34)c2O1)N1CCN(c2cccnc2)CC1 ; O=C([C@@H]1Cc2cc(Cl)cc(-c3ccnc4[nH]ccc34)c2O1)N1CCN(c2cccnc2)CC1]

### (R)-(5-chloro-7-(1H-pyrrolo[2,3-b]pyridin-4-yl)-2,3-dihydrobenzofuran-2-yl)(4-(pyridin-3-yl)piperazin-1-yl)methanone and (S)-(5-chloro-7-(1H-pyrrolo[2,3-b]pyridin-4-yl)-2,3-dihydrobenzofuran-2-yl)(4-(pyridin-3-yl)piperazin-1-yl)methanone

The example was prepared using precursor I (step 5), general procedures A and C. The final product was converted to HCl salt using 4M HCl in dioxane (1 mL). ¹H NMR (400 MHz, CD₃OD, hydrochloric acid): δ 8.42 (dd, J=6.1, 4.2 Hz, 1H), 8.39 (d, J=2.6 Hz, 1H), 8.17 (d, J=5.3 Hz, 1H), 8.09 (dd, J=8.2, 2.9 Hz, 1H), 7.94-7.84 (m, 1H), 7.77 (d, J=6.1 Hz, 1H), 7.69 (d, J=3.6 Hz, 1H), 7.61-7.52 (m, 1H), 7.49-7.45 (m, 1H), 6.99 (d, J=3.6 Hz, 1H), 5.91 (dd, J=8.8, 7.7 Hz, 1H), 3.93-3.39 (m, 10H). [M+H] 460.0.

### Example 68

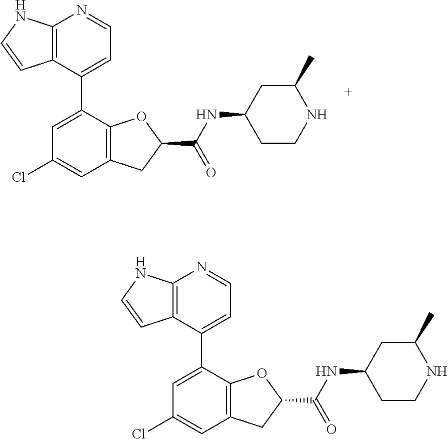 [SMILES: C[C@@H]1C[C@H](NC(=O)[C@H]2Cc3cc(Cl)cc(-c4ccnc5[nH]ccc45)c3O2)CCN1 ; C[C@@H]1C[C@H](NC(=O)[C@@H]2Cc3cc(Cl)cc(-c4ccnc5[nH]ccc45)c3O2)CCN1]

### (R)-5-chloro-N-((2R,4R)-2-methylpiperidin-4-yl)-7-(1H-pyrrolo[2,3-b]pyridin-4-yl)-2,3-dihydrobenzofuran-2-carboxamide and (S)-5-chloro-N-((2R,4R)-2-methylpiperidin-4-yl)-7-(1H-pyrrolo[2,3-b]pyridin-4-yl)-2,3-dihydrobenzofuran-2-carboxamide

The example was prepared using precursor I (step 5), general procedures A, C, and D. ¹H NMR (400 MHz, CD₃OD, hydrochloric acid salt): δ 8.45 (d, J=6.3 Hz, 1H), 8.35 (d, J=7.6 Hz, 1H), 7.82 (d, J=6.3 Hz, 1H), 7.76 (d, J=3.5 Hz, 1H), 7.56 (d, J=2.0 Hz, 1H), 7.47 (s, 1H), 7.00 (d, J=3.5 Hz, 1H), 5.32 (dd, J=10.3, 7.1 Hz, 1H), 4.08-3.90 (m, 1H), 3.78-3.66 (m, 1H), 3.48-3.35 (m, 2H), 3.10 (ddd, J=15.9, 8.3, 2.1 Hz, 2H), 2.12 (t, J=14.0 Hz, 2H), 1.82-1.40 (m, 2H), 1.34 (dd, J=6.5, 1.8 Hz, 3H). [M+H] 411.1.

### Example 69

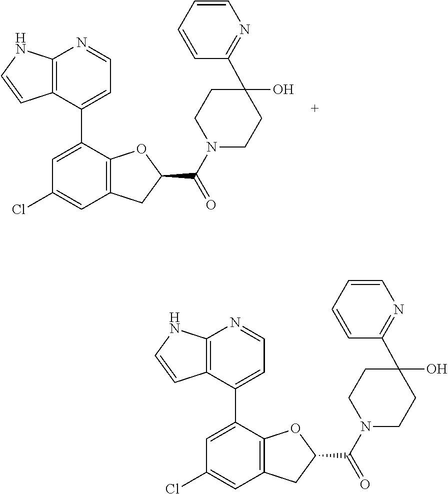 [SMILES: O=C([C@H]1Cc2cc(Cl)cc(-c3ccnc4[nH]ccc34)c2O1)N1CCC(O)(c2ccccn2)CC1 ; O=C([C@@H]1Cc2cc(Cl)cc(-c3ccnc4[nH]ccc34)c2O1)N1CCC(O)(c2ccccn2)CC1]

### (R)-(5-chloro-7-(1H-pyrrolo[2,3-b]pyridin-4-yl)-2,3-dihydrobenzofuran-2-yl)(4-hydroxy-4-(pyridin-2-yl)piperidin-1-yl)methanone and (S)-(5-chloro-7-(1H-pyrrolo[2,3-b]pyridin-4-yl)-2,3-dihydrobenzofuran-2-yl)(4-hydroxy-4-(pyridin-2-yl)piperidin-1-yl)methanone

The example was prepared using precursor I (step 5), general procedures A and C. The final product was converted to HCl salt using 4M HCl in dioxane. ¹H NMR (400 MHz, CD₃OD, hydrochloric acid salt): δ 8.79-8.72 (m, 1H), 8.60 (dtd, J=17.2, 8.1, 1.5 Hz, 1H), 8.42 (dd, J=17.7, 6.2 Hz, 1H), 8.10 (dd, J=118.5, 8.3 Hz, 1H), 8.02 (dd, J=14.6, 7.6 Hz, 1H), 7.87 (d, J=6.3 Hz, 1H), 7.74 (dd, J=8.3, 3.6 Hz, 1H), 7.58 (s, 1H), 7.52-7.45 (m, 1H), 7.10 (dd, J=18.2, 3.6 Hz, 1H), 5.98-5.89 (m, 1H), 4.59 (t, J=10.6 Hz, 1H), 4.05 (d, J=14.1 Hz, 1H), 3.81-3.53 (m, 3H), 3.22-3.10 (m, 1H), 2.37-1.69 (m, 4H). [M+H] 475.0.

### Example 70

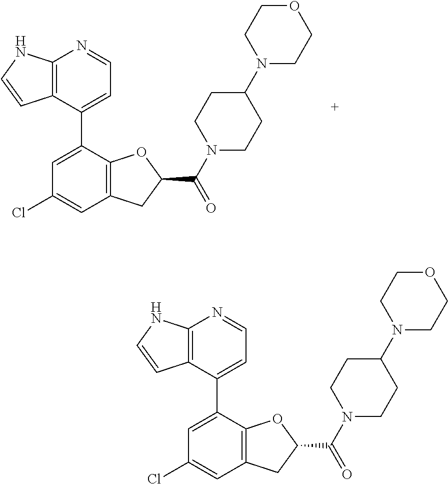 [SMILES: O=C([C@H]1Cc2cc(Cl)cc(-c3ccnc4[nH]ccc34)c2O1)N1CCC(N2CCOCC2)CC1 ; O=C([C@@H]1Cc2cc(Cl)cc(-c3ccnc4[nH]ccc34)c2O1)N1CCC(N2CCOCC2)CC1]

### (R)-(5-chloro-7-(1H-pyrrolo[2,3-b]pyridin-4-yl)-2,3-dihydrobenzofuran-2-yl)(4-morpholinopiperidin-1-yl)methanone and (S)-(5-chloro-7-(1H-pyrrolo[2,3-b]pyridin-4-yl)-2,3-dihydrobenzofuran-2-yl)(4-morpholinopiperidin-1-yl)methanone

The example was prepared using precursor I (step 5), general procedures A and C. The final product was converted to HCl salt using 4M HCl in dioxane. ¹H NMR (400 MHz, CD₃OD, hydrochloric acid salt): δ 8.44 (dd, J=12.8, 6.2 Hz, 1H), 7.81 (dd, J=6.0, 2.3 Hz, 1H), 7.73 (t, J=3.1 Hz, 1H), 7.56 (s, 1H), 7.47 (d, J=6.2 Hz, 1H), 7.03 (dd, J=15.4, 3.6 Hz, 1H), 5.94-5.82 (m, 1H), 4.70 (t, J=12.3 Hz, 1H), 4.28-4.17 (m, 1H), 4.08 (t, J=14.3 Hz, 2H), 3.91-3.74 (m, 2H), 3.64-3.58 (m, 1H), 3.57-3.43 (m, 3H), 3.43-3.00 (m, 4H), 2.82-2.70 (m, 1H), 2.34-2.08 (m, 2H), 1.86-1.43 (m, 2H). [M+H] 467.0.

### Example 71

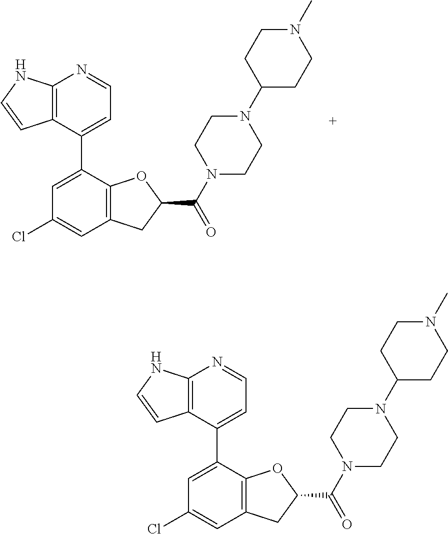 [SMILES: CN1CCC(N2CCN(C(=O)[C@H]3Cc4cc(Cl)cc(-c5ccnc6[nH]ccc56)c4O3)CC2)CC1 ; CN1CCC(N2CCN(C(=O)[C@@H]3Cc4cc(Cl)cc(-c5ccnc6[nH]ccc56)c4O3)CC2)CC1]

### (R)-(5-chloro-7-(1H-pyrrolo[2,3-b]pyridin-4-yl)-2,3-dihydrobenzofuran-2-yl)(4-(1-methylpiperidin-4-yl)piperazin-1-yl)methanone and (S)-(5-chloro-7-(1H-pyrrolo[2,3-b]pyridin-4-yl)-2,3-dihydrobenzofuran-2-yl)(4-(1-methylpiperidin-4-yl)piperazin-1-yl)methanone

The example was prepared using precursor I (step 5), general procedures A and C. The final product was converted to HCl salt using 4M HCl in dioxane. ¹H NMR (400 MHz, CD₃OD, hydrochloric acid salt): δ 8.48 (d, J=6.2 Hz, 1H), 7.83 (d, J=6.1 Hz, 1H), 7.78 (d, J=3.5 Hz, 1H), 7.57 (d, J=2.1 Hz, 1H), 7.49 (s, 1H), 7.02 (bs, 1H), 5.92-5.85 (m, 1H), 4.78-3.94 (m, 3H), 3.86-3.38 (m, 8H), 3.16 (dd, J=20.1, 7.4 Hz, 4H), 2.91 (s, 3H), 2.47 (d, J=8.7 Hz, 2H), 2.16 (dd, J=23.3, 12.6 Hz, 2H). [M+H] 480.0.

### Example 72

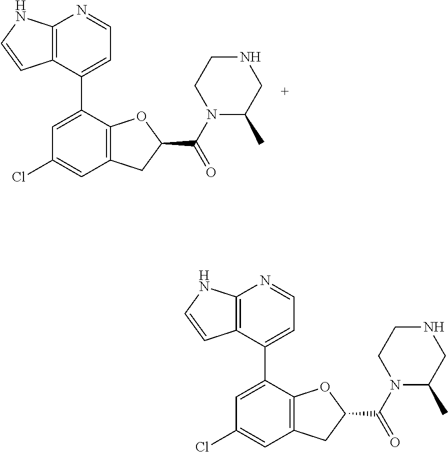 [SMILES: C[C@@H]1CNCCN1C(=O)[C@H]1Cc2cc(Cl)cc(-c3ccnc4[nH]ccc34)c2O1 ; C[C@@H]1CNCCN1C(=O)[C@@H]1Cc2cc(Cl)cc(-c3ccnc4[nH]ccc34)c2O1]

### ((R)-5-chloro-7-(1H-pyrrolo[2,3-b]pyridin-4-yl)-2,3-dihydrobenzofuran-2-yl)((R)-2-methylpiperazin-1-yl)methanone and ((S)-5-chloro-7-(1H-pyrrolo[2,3-b]pyridin-4-yl)-2,3-dihydrobenzofuran-2-yl)((R)-2-methylpiperazin-1-yl)methanone

The example was prepared using precursor I (step 5), general procedures A, C, and D. ¹H NMR (400 MHz, CD₃OD, hydrochloric acid salt): δ 8.13 (s, 1H), 7.48 (d, J=6.0 Hz, 1H), 7.42 (d, J=3.6 Hz, 1H), 7.25 (d, J=2.1 Hz, 1H), 7.17 (s, 1H), 6.72 (d, J=20.3 Hz, 1H), 5.55 (m, 1H), 4.26 (s, 2H), 3.80 (d, J=7.3 Hz, 1H), 3.36 (s, 2H), 3.31-3.20 (m, 1H), 2.93-2.51 (m, 4H), 1.30-0.94 (m, 2H). [M+H] 397.0.

### Example 73

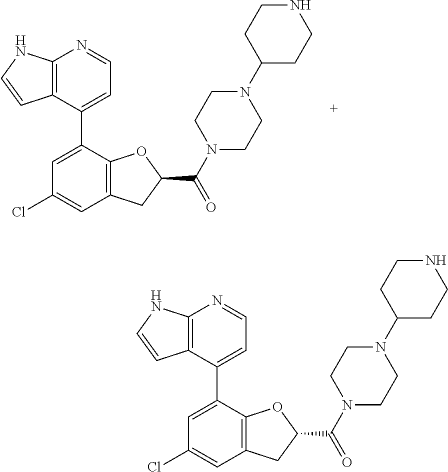 [SMILES: O=C([C@H]1Cc2cc(Cl)cc(-c3ccnc4[nH]ccc34)c2O1)N1CCN(C2CCNCC2)CC1 ; O=C([C@@H]1Cc2cc(Cl)cc(-c3ccnc4[nH]ccc34)c2O1)N1CCN(C2CCNCC2)CC1]

### (R)-(5-chloro-7-(1H-pyrrolo[2,3-b]pyridin-4-yl)-2,3-dihydrobenzofuran-2-yl)(4-(piperidin-4-yl)piperazin-1-yl)methanone and (S)-(5-chloro-7-(1H-pyrrolo[2,3-b]pyridin-4-yl)-2,3-dihydrobenzofuran-2-yl)(4-(piperidin-4-yl)piperazin-1-yl)methanone

The example was prepared using precursor I (step 5), general procedures A, C, and D. ¹H NMR (400 MHz, CD₃OD, hydrochloric acid salt): δ 8.46 (d, J=6.2 Hz, 1H), 7.86-7.71 (m, 2H), 7.56 (d, J=2.1 Hz, 1H), 7.48 (s, 1H), 6.99 (s, 1H), 5.94-5.82 (m, 1H), 4.77-4.06 (m, 3H), 3.91-3.39 (m, 8H), 3.13 (dd, J=14.1, 12.4 Hz, 3H), 3.02-2.60 (m, 1H), 2.41 (s, 2H), 2.05 (d, J=12.4 Hz, 2H). [M+H] 466.0.

### Example 74

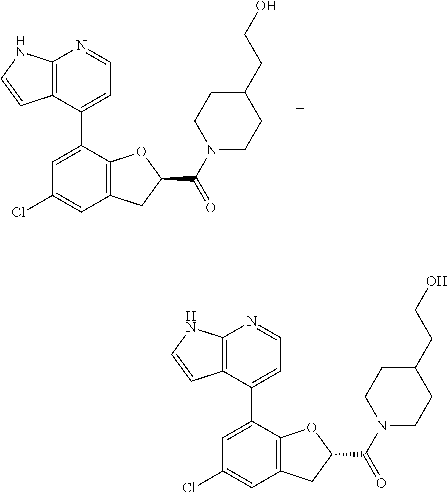 [SMILES: O=C([C@H]1Cc2cc(Cl)cc(-c3ccnc4[nH]ccc34)c2O1)N1CCC(CCO)CC1 ; O=C([C@@H]1Cc2cc(Cl)cc(-c3ccnc4[nH]ccc34)c2O1)N1CCC(CCO)CC1]

### (R)-(5-chloro-7-(1H-pyrrolo[2,3-b]pyridin-4-yl)-2,3-dihydrobenzofuran-2-yl)(4-(2-hydroxyethyl)piperidin-1-yl)methanone and (S)-(5-chloro-7-(1H-pyrrolo[2,3-b]pyridin-4-yl)-2,3-dihydrobenzofuran-2-yl)(4-(2-hydroxyethyl)piperidin-1-yl)methanone

The example was prepared using precursor I (step 5), general procedures A and C. The final product was converted to HCl salt using 4M HCl in dioxane. ¹H NMR (400 MHz, CD₃OD, hydrochloric acid salt): δ 8.42 (dd, J=6.2, 1.9 Hz, 1H), 7.81 (dd, J=8.8, 6.2 Hz, 1H), 7.72 (d, J=3.4 Hz, 1H), 7.57-7.51 (m, 1H), 7.47 (s, 1H), 7.02 (dd, J=7.5, 3.6 Hz, 1H), 5.88-5.79 (m, 1H), 4.48 (d, J=13.7 Hz, 1H), 4.00 (d, J=12.9 Hz, 1H), 3.66-3.54 (m, 3H), 3.17-3.03 (m, 2H), 2.81-2.60 (m, 1H), 1.61 (m, 4H), 1.46-1.23 (m, 2H), 1.14-0.84 (m, 1H). [M+H] 426.0.

### Example 75

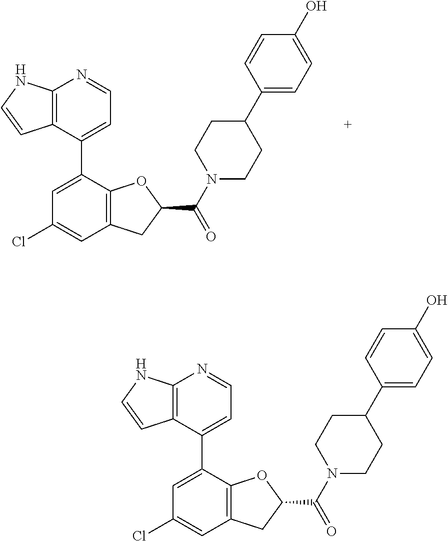 [SMILES: O=C([C@H]1Cc2cc(Cl)cc(-c3ccnc4[nH]ccc34)c2O1)N1CCC(c2ccc(O)cc2)CC1 ; O=C([C@@H]1Cc2cc(Cl)cc(-c3ccnc4[nH]ccc34)c2O1)N1CCC(c2ccc(O)cc2)CC1]

### (R)-(5-chloro-7-(1H-pyrrolo[2,3-b]pyridin-4-yl)-2,3-dihydrobenzofuran-2-yl)(4-(4-hydroxyphenyl)piperidin-1-yl)methanone and (S)-(5-chloro-7-(1H-pyrrolo[2,3-b]pyridin-4-yl)-2,3-dihydrobenzofuran-2-yl)(4-(4-hydroxyphenyl)piperidin-1-yl)methanone

The example was prepared using precursor I (step 5), general procedures A and C. The final product was converted to HCl salt using 4M HCl in dioxane. ¹H NMR (400 MHz, CD₃OD, hydrochloric acid salt): δ 7.86 (t, J=6.2 Hz, 1H), 7.78-7.72 (m, 2H), 7.57-7.55 (m, 1H), 7.50 (d, J=2.1 Hz, 1H), 7.10-7.03 (m, 1H), 6.74 (ddd, J=32.5, 20.7, 8.6 Hz, 4H), 5.94-5.85 (m, 1H), 4.70-4.54 (m, 1H), 4.19-4.04 (m, 1H), 3.77-3.56 (m, 4H), 2.89-2.61 (m, 2H), 1.93-1.20 (m, 3H). [M+H] 474.0.

### Example 76

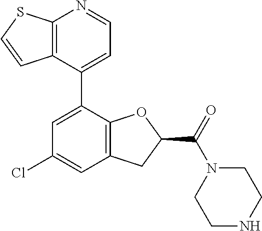 [SMILES: O=C([C@H]1Cc2cc(Cl)cc(-c3ccnc4sccc34)c2O1)N1CCNCC1]

### (R)-(5-chloro-7-(thieno[2,3-b]pyridin-4-yl)-2,3-dihydrobenzofuran-2-yl)(piperazin-1-yl)methanone

The example was prepared using precursor II, general procedures C and D as the HCl salt with 4-chlorothieno[2,3-b]pyridine. ¹H NMR (400 MHz, CD₃OD, hydrochloric acid salt): δ ppm 8.80 (d, J=5.0 Hz, 1H), 7.97 (d, J=5.7 Hz, 1H), 7.85 (d, J=4.4 Hz, 1H), 7.67 (d, J=5.7 Hz, 1H), 7.47 (d, J=9.0 Hz, 2H), 5.82 (s, 1H), 3.80 (dd, J=54.9, 34.7 Hz, 5H), 3.62 (dd, J=14.7, 7.7 Hz, 1H), 3.28-3.24 (m, 2H), 3.16 (s, 3H), 2.96 (s, 2H). LCMS: [M+H⁺]=400.1.

### Example 77

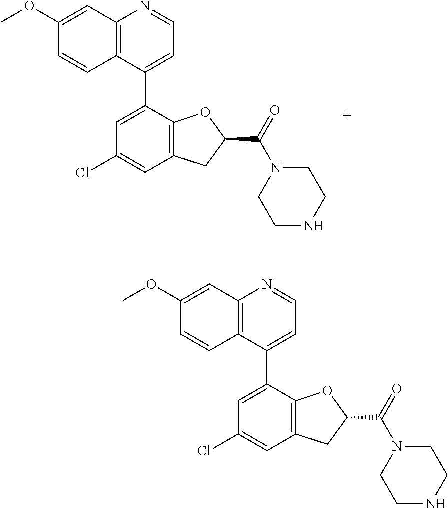 [SMILES: COc1ccc2c(-c3cc(Cl)cc4c3O[C@@H](C(=O)N3CCNCC3)C4)ccnc2c1 ; COc1ccc2c(-c3cc(Cl)cc4c3O[C@H](C(=O)N3CCNCC3)C4)ccnc2c1]

### (R)-(5-chloro-7-(7-methoxyquinolin-4-yl)-2,3-dihydrobenzofuran-2-yl)(piperazin-1-yl)methanone and (S)-(5-chloro-7-(7-methoxyquinolin-4-yl)-2,3-dihydrobenzofuran-2-yl)(piperazin-1-yl)methanone

The example was prepared using precursor I, general procedures C and D as the HCl salt with 4-bromo-7-methoxy-quinoline. ¹H NMR (400 MHz, CD₃OD, hydrochloric acid salt): δ ppm 9.13-9.00 (m, 1H), 8.27-8.13 (m, 1H), 7.96 (s, 1H), 7.65 (s, 1H), 7.56 (t, J=7.6 Hz, 2H), 7.42 (s, 1H), 5.88-5.74 (m, 1H), 4.12 (d, J=1.3 Hz, 3H), 3.90-3.59 (m, 6H), 3.58-3.45 (m, 3H), 3.26-2.98 (m, 2H). LCMS: [M+H⁺]=424.1.

### Example 78

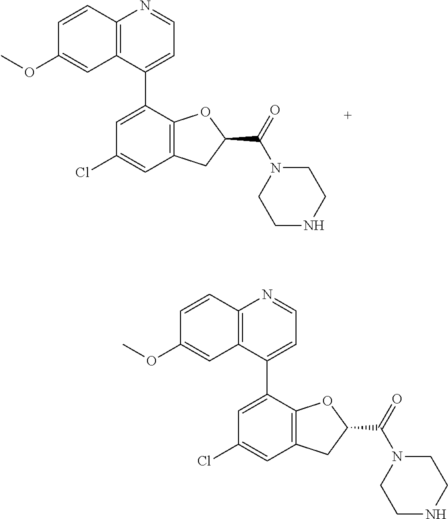 [SMILES: COc1ccc2nccc(-c3cc(Cl)cc4c3O[C@@H](C(=O)N3CCNCC3)C4)c2c1 ; COc1ccc2nccc(-c3cc(Cl)cc4c3O[C@H](C(=O)N3CCNCC3)C4)c2c1]

### (R)-(5-chloro-7-(6-methoxyquinolin-4-yl)-2,3-dihydrobenzofuran-2-yl)(piperazin-1-yl)methanone and (S)-(5-chloro-7-(6-methoxyquinolin-4-yl)-2,3-dihydrobenzofuran-2-yl)(piperazin-1-yl)methanone

The example was prepared using precursor I, general procedures C and D as the HCl salt with 4-bromo-4-methoxy-quinoline. ¹H NMR (400 MHz, CD₃OD, hydrochloric acid salt): δ ppm 8.93 (d, J=5.2 Hz, 1H), 8.12 (d, J=9.3 Hz, 1H), 7.95 (s, 1H), 7.79-7.65 (m, 1H), 7.57-7.38 (m, 2H), 7.34-7.18 (m, 2H), 7.11 (d, J=8.9 Hz, 1H), 5.66 (d, J=7.8 Hz, 1H), 3.82 (s, 3H), 3.59 (dd, J=54.4, 34.0 Hz, 6H), 3.02 (s, 3H), 2.75 (s, 1H), 2.50 (s, 1H). LCMS: [M+H⁺]=424.1.

### Example 79

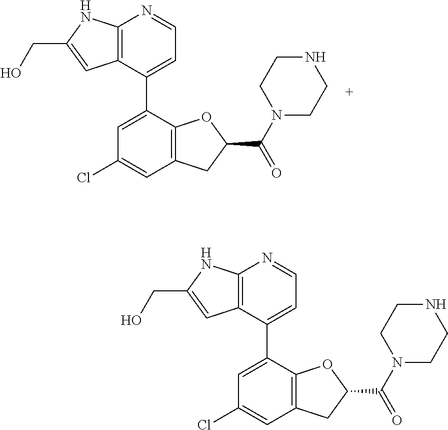 [SMILES: O=C([C@H]1Cc2cc(Cl)cc(-c3ccnc4[nH]c(CO)cc34)c2O1)N1CCNCC1 ; O=C([C@@H]1Cc2cc(Cl)cc(-c3ccnc4[nH]c(CO)cc34)c2O1)N1CCNCC1]

### (R)-(5-chloro-7-(2-(hydroxymethyl)-1H-pyrrolo[2,3-b]pyridin-4-yl)-2,3-dihydrobenzofuran-2-yl)(piperazin-1-yl)methanone and (S)-(5-chloro-7-(2-(hydroxymethyl)-1H-pyrrolo[2,3-b]pyridin-4-yl)-2,3-dihydrobenzofuran-2-yl)(piperazin-1-yl)methanone

The example was prepared using general procedure B with (4-chloro-1-(phenylsulfonyl)-1H-pyrrolo[2,3-b]pyridin-2-yl)methanol and purified by reverse phase HPLC using water (0.1% TFA) and acetonitrile (0.1% TFA) as the eluent followed by using general procedures D. [M+H] 413.0.

### Example 80

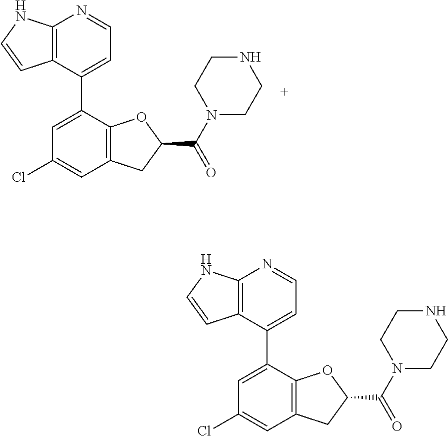 [SMILES: O=C([C@H]1Cc2cc(Cl)cc(-c3ccnc4[nH]ccc34)c2O1)N1CCNCC1 ; O=C([C@@H]1Cc2cc(Cl)cc(-c3ccnc4[nH]ccc34)c2O1)N1CCNCC1]

### (R)-(5-chloro-7-(1H-pyrrolo[2,3-b]pyridin-4-yl)-2,3-dihydrobenzofuran-2-yl)(piperazin-1-yl)methanone and (S)-(5-chloro-7-(1H-pyrrolo[2,3-b]pyridin-4-yl)-2,3-dihydrobenzofuran-2-yl)(piperazin-1-yl)methanone

The example was prepared using general procedure C with 4-(4,4,5,5-tetramethyl-1,3,2-dioxaborolan-2-yl)-1H-pyrrolo[2,3-b]pyridine and purified by reverse phase HPLC using water (0.1% TFA) and acetonitrile (0.1% TFA) as the eluent followed by using general procedures D and F. ¹H NMR (400 MHz, CD₃OD, free base): δ ppm 8.23 (bs, 1H), 7.43 (d, J=3.6 Hz, 1H), 7.40 (d, J=2 Hz, 1H), 7.32 (s, 1H), 7.28 (s, 1H), 6.55 (d, J=3.6 Hz, 1H), 5.69 (dd, J=9.6, 6.8 Hz, 1H), 3.76-2.56 (m, 10H). [M+H] 383.0.

### Example 81

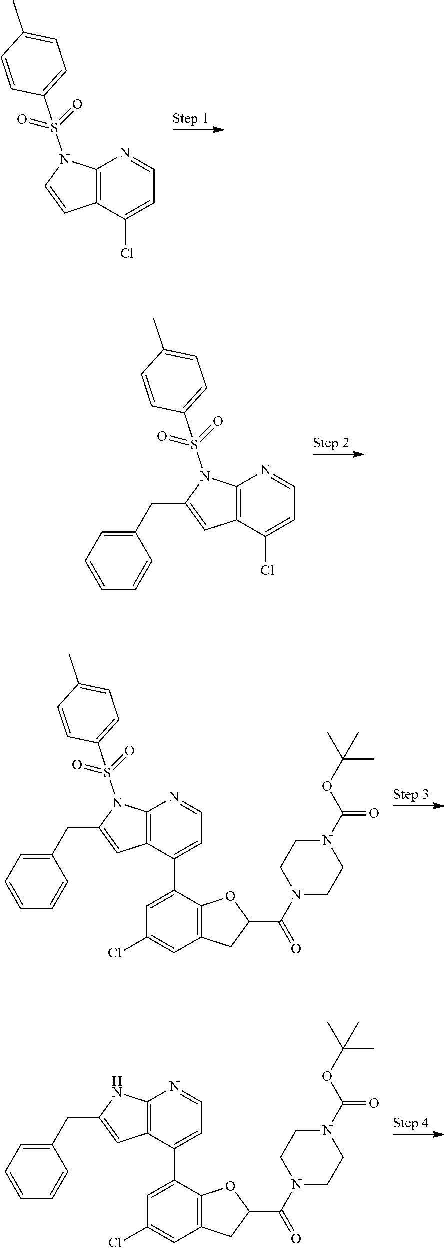 [SMILES: Cc1ccc(S(=O)(=O)n2ccc3c(Cl)ccnc32)cc1 ; Cc1ccc(S(=O)(=O)n2c(Cc3ccccc3)cc3c(Cl)ccnc32)cc1 ; Cc1ccc(S(=O)(=O)n2c(Cc3ccccc3)cc3c(-c4cc(Cl)cc5c4OC(C(=O)N4CCN(C(=O)OC(C)(C)C)CC4)C5)ccnc32)cc1 ; CC(C)(C)OC(=O)N1CCN(C(=O)C2Cc3cc(Cl)cc(-c4ccnc5[nH]c(Cc6ccccc6)cc45)c3O2)CC1]

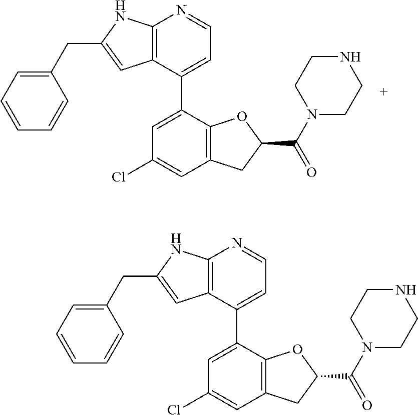 [SMILES: O=C([C@H]1Cc2cc(Cl)cc(-c3ccnc4[nH]c(Cc5ccccc5)cc34)c2O1)N1CCNCC1 ; O=C([C@@H]1Cc2cc(Cl)cc(-c3ccnc4[nH]c(Cc5ccccc5)cc34)c2O1)N1CCNCC1]

### Step 1: 2-benzyl-4-chloro-1-tosyl-1H-pyrrolo[2,3-b]pyridine

LDA (2.0M in THF, 1.8 mL, 3.6 mmol) was added dropwise to 4-chloro-1-tosyl-1H-pyrrolo[2,3-b]pyridine 920 mg, 3 mmol) in THF (15 mL) at −78° C. The reaction mixture was stirred at the same temperature for 15 minutes and benzyl bromide (667 mg, 3.9 mmol) was added. The reaction was warmed up to rt over 1 hour, quenched by addition saturated NH₄Cl, and extracted with EtOAc. The organic fraction was concentrated under vacuum and the crude material purified by column chromatography (0-20% EtOAc, Hexanes) to give the title compound.

### Step 2: tert-butyl 4-(7-(2-benzyl-1-tosyl-1H-pyrrolo[2,3-b]pyridin-4-yl)-5-chloro-2,3-dihydrobenzofuran-2-carbonyl)piperazine-1-carboxylate

The title compound was prepared following general procedure B and purified by column chromatography (0-20% EtOAc, Hexanes).

### Step 3: tert-butyl 4-(7-(2-benzyl-1H-pyrrolo[2,3-b]pyridin-4-yl)-5-chloro-2,3-dihydrobenzofuran-2-carbonyl)piperazine-1-carboxylate

tert-butyl 4-(7-(2-benzyl-1-tosyl-1H-pyrrolo[2,3-b]pyridin-4-yl)-5-chloro-2,3-dihydrobenzofuran-2-carbonyl)piperazine-1-carboxylate (47 mg, 0.064 mmol) was dissolved in THF (0.5 mL)/MeOH (0.5 mL) and Cs₂CO₃ (84 mg, 0.26 mmol) was added. The reaction was stirred at 35° C. for 1 h, diluted with EtOAc (5 mL) and filtered through a short plug of silica gel. The solution was concentrated and the crude material purified by flash chromatography to give the title compound.

### Step 4: (R)-(7-(2-benzyl-1H-pyrrolo[2,3-b]pyridin-4-yl)-5-chloro-2,3-dihydrobenzofuran-2-yl)(piperazin-1-yl)methanone and (S)-(7-(2-benzyl-1H-pyrrolo[2,3-b]pyridin-4-yl)-5-chloro-2,3-dihydrobenzofuran-2-yl)(piperazin-1-yl)methanone

The title compound was prepared following general procedures D and F successively. [M+H^{+]} 473.0.

### Example 82

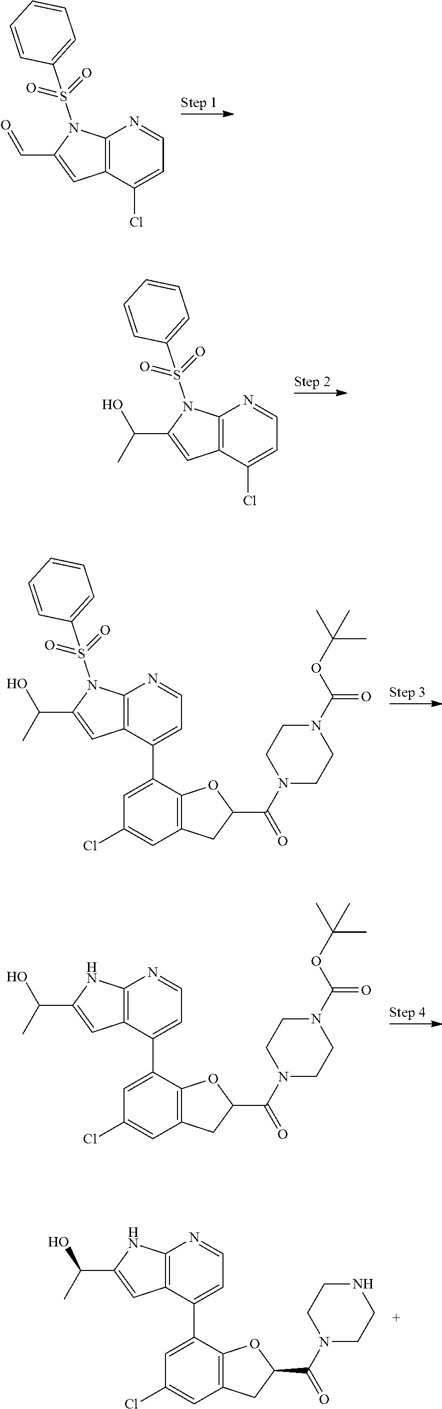 [SMILES: O=Cc1cc2c(Cl)ccnc2n1S(=O)(=O)c1ccccc1 ; CC(O)c1cc2c(Cl)ccnc2n1S(=O)(=O)c1ccccc1 ; CC(O)c1cc2c(-c3cc(Cl)cc4c3OC(C(=O)N3CCN(C(=O)OC(C)(C)C)CC3)C4)ccnc2n1S(=O)(=O)c1ccccc1 ; CC(O)c1cc2c(-c3cc(Cl)cc4c3OC(C(=O)N3CCN(C(=O)OC(C)(C)C)CC3)C4)ccnc2[nH]1 ; C[C@@H](O)c1cc2c(-c3cc(Cl)cc4c3O[C@@H](C(=O)N3CCNCC3)C4)ccnc2[nH]1]

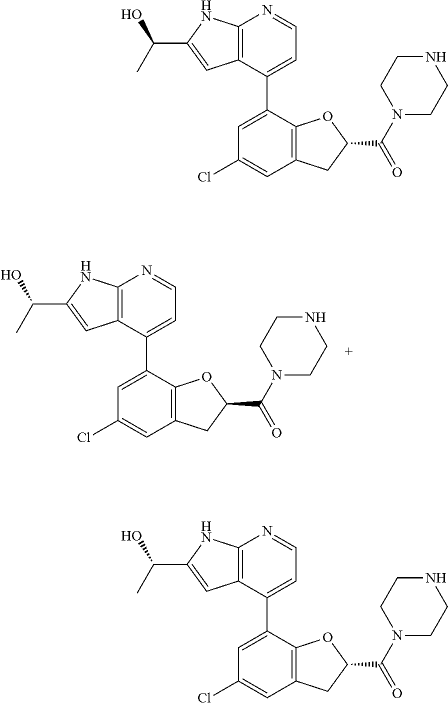 [SMILES: C[C@@H](O)c1cc2c(-c3cc(Cl)cc4c3O[C@H](C(=O)N3CCNCC3)C4)ccnc2[nH]1 ; C[C@H](O)c1cc2c(-c3cc(Cl)cc4c3O[C@@H](C(=O)N3CCNCC3)C4)ccnc2[nH]1 ; C[C@H](O)c1cc2c(-c3cc(Cl)cc4c3O[C@H](C(=O)N3CCNCC3)C4)ccnc2[nH]1]

### Step 1: 1-(4-chloro-1-(phenylsulfonyl)-1H-pyrrolo[2,3-b]pyridin-2-yl)ethan-1-ol

MeMgBr (1M in THF, 1.1 mL, 0.33 mmol) was added dropwise to 4-chloro-1-(phenylsulfonyl)-1H-pyrrolo[2,3-b]pyridine-2-carbaldehyde (96 mg, 0.3 mmol) in THF (4 mL) at 0° C. The reaction was concentrated under vacuum and the crude material purified by flash chromatography (40% EtOAc/hexanes) to give the title compound.

### Step 2: tert-butyl 4-(5-chloro-7-(2-(1-hydroxyethyl)-1-(phenylsulfonyl)-1H-pyrrolo[2,3-b]pyridin-4-yl)-2,3-dihydrobenzofuran-2-carbonyl)piperazine-1-carboxylate

The title compound was prepared following general procedure B and purified by column chromatography (0-20% EtOAc, Hexanes).

### Step 3: tert-butyl 4-(5-chloro-7-(2-(1-hydroxyethyl)-1H-pyrrolo[2,3-b]pyridin-4-yl)-2,3-dihydrobenzofuran-2-carbonyl)piperazine-1-carboxylate

tert-butyl 4-(5-chloro-7-(2-(1-hydroxyethyl)-1-(phenylsulfonyl)-1H-pyrrolo[2,3-b]pyridin-4-yl)-2,3-dihydrobenzofuran-2-carbonyl)piperazine-1-carboxylate (50 mg, 0.075 mmol) was dissolved in THF (0.5 mL)/MeOH (0.5 mL) and Cs₂CO₃ (122 mg, 0.38 mmol) was added. The reaction was stirred at 35° C. for 1 h, diluted with EtOAc (5 mL) and filtered through a short plug of silica gel. The solution was concentrated and the crude material purified by flash chromatography (10% MeOH/DCM) to give the title compound.

### Step 4: ((R)-5-chloro-7-(2-((S)-1-hydroxyethyl)-1H-pyrrolo[2,3-b]pyridin-4-yl)-2,3-dihydrobenzofuran-2-yl)(piperazin-1-yl)methanone and ((R)-5-chloro-7-(2-((R)-1-hydroxyethyl)-1H-pyrrolo[2,3-b]pyridin-4-yl)-2,3-dihydrobenzofuran-2-yl)(piperazin-1-yl)methanone and ((S)-5-chloro-7-(2-((R)-1-hydroxyethyl)-1H-pyrrolo[2,3-b]pyridin-4-yl)-2,3-dihydrobenzofuran-2-yl)(piperazin-1-yl)methanone and ((S)-5-chloro-7-(2-((S)-1-hydroxyethyl)-1H-pyrrolo[2,3-b]pyridin-4-yl)-2,3-dihydrobenzofuran-2-yl)(piperazin-1-yl)methanone

The title compounds was prepared following general procedures D and F successively. ¹H NMR (400 MHz, CD₃OD) δ 8.36 (d, J=5.7 Hz, 1H), 7.79 (d, J=5.7 Hz, 1H), 7.54 (s, 1H), 7.47 (s, 1H), 6.90 (d, J=2.7 Hz, 1H), 5.96-5.80 (m, 1H), 5.16-5.04 (m, 1H), 3.99-3.73 (m, 4H), 3.30-3.05 (m, 6H), 1.67-1.57 (m, 3H). [M+H⁺] 427.0.

### Example 83

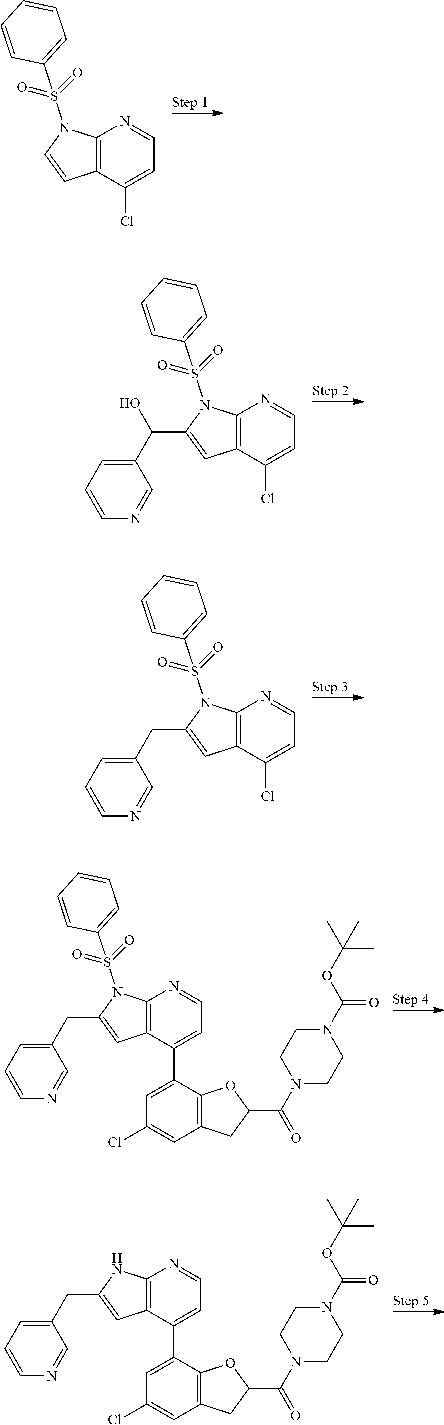 [SMILES: O=S(=O)(c1ccccc1)n1ccc2c(Cl)ccnc21 ; O=S(=O)(c1ccccc1)n1c(C(O)c2cccnc2)cc2c(Cl)ccnc21 ; O=S(=O)(c1ccccc1)n1c(Cc2cccnc2)cc2c(Cl)ccnc21 ; CC(C)(C)OC(=O)N1CCN(C(=O)C2Cc3cc(Cl)cc(-c4ccnc5c4cc(Cc4cccnc4)n5S(=O)(=O)c4ccccc4)c3O2)CC1 ; CC(C)(C)OC(=O)N1CCN(C(=O)C2Cc3cc(Cl)cc(-c4ccnc5[nH]c(Cc6cccnc6)cc45)c3O2)CC1]

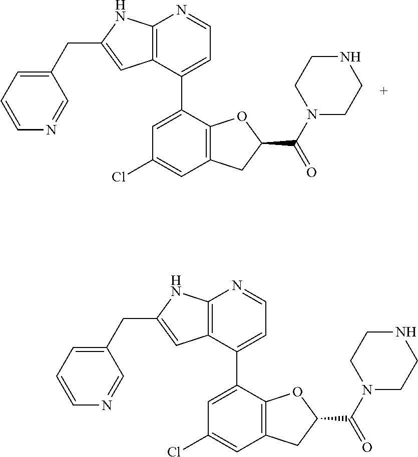 [SMILES: O=C([C@H]1Cc2cc(Cl)cc(-c3ccnc4[nH]c(Cc5cccnc5)cc34)c2O1)N1CCNCC1 ; O=C([C@@H]1Cc2cc(Cl)cc(-c3ccnc4[nH]c(Cc5cccnc5)cc34)c2O1)N1CCNCC1]

### Step 1: (4-chloro-1-(phenylsulfonyl)-1H-pyrrolo[2,3-b]pyridin-2-yl)(pyridin-3-yl)methanol

To a solution of 4-chloro-1-(phenylsulfonyl)-1H-pyrrolo[2,3-b]pyridine (300 mg, 1.02 mmol) in anhydrous THF (5 mL) at −50° C. was added LDA (1M in THF/hexanes, 1.05 eq) dropwise. The reaction was stirred at −50° C. for 30 min before nicotinaldehyde (0.12 mL, 1.1 eq) was added dropwise. The reaction was allowed to slowly warm up from −50° C. to −10° C. while stirring for 1 hr and was quenched with NH₄Cl solution. The reaction mixture was extracted with EtOAc and washed with H₂O, NaHCO₃ solution and brine. The crude was concentrated and purified with flash chromatography (0-35% EtOAc in DCM) to give the title compound (300 mg, 75%).

### Step 2: 4-chloro-1-(phenylsulfonyl)-2-(pyridin-3-ylmethyl)-1H-pyrrolo[2,3-b]pyridine

To a solution of (4-chloro-1-(phenylsulfonyl)-1H-pyrrolo[2,3-b]pyridin-2-yl)(pyridin-3-yl)methanol (130 mg, 0.32 mmol) in anhydrous DCM (5 mL) was added SOCl₂ (70 ul, 3 eq). The reaction was stirred for 20 min at rt and concentrated in vacu and dried on high vacuum for 30 min. The crude was dissolved in AcOH (4 mL) and treated with Zn (0.8 g, excessive) and stirred vigorously at rt for 3 hr. The mixture was diluted with DCM and filtered to give the title compound without further purification.

### Step 3: tert-butyl 4-(5-chloro-7-(1-(phenylsulfonyl)-2-(pyridin-3-ylmethyl)-1H-pyrrolo[2,3-b]pyridin-4-yl)-2,3-dihydrobenzofuran-2-carbonyl)piperazine-1-carboxylate

The title compound was prepared following general procedure B and purified by column chromatography (0-20% EtOAc, Hexanes).

### Step 4: tert-butyl 4-(5-chloro-7-(2-(pyridin-3-ylmethyl)-1H-pyrrolo[2,3-b]pyridin-4-yl)-2,3-dihydrobenzofuran-2-carbonyl)piperazine-1-carboxylate

tert-butyl 4-(5-chloro-7-(1-(phenylsulfonyl)-2-(pyridin-3-ylmethyl)-1H-pyrrolo[2,3-b]pyridin-4-yl)-2,3-dihydrobenzofuran-2-carbonyl)piperazine-1-carboxylate (50 mg, 0.066 mmol) was dissolved in THF (0.5 mL)/MeOH (0.5 mL) and Cs₂CO₃ (122 mg, 0.38 mmol) was added. The reaction was stirred at 35° C. for 1 h, diluted with EtOAc (5 mL) and filtered through a short plug of silica gel. The solution was concentrated and the crude material purified by flash chromatography (10% MeOH/DCM) to give the title compound.

### Step 5: (R)-(5-chloro-7-(2-(pyridin-3-ylmethyl)-1H-pyrrolo[2,3-b]pyridin-4-yl)-2,3-dihydrobenzofuran-2-yl)(piperazin-1-yl)methanone and (S)-(5-chloro-7-(2-(pyridin-3-ylmethyl)-1H-pyrrolo[2,3-b]pyridin-4-yl)-2,3-dihydrobenzofuran-2-yl)(piperazin-1-yl)methanone

The title compounds were prepared following general procedures D and F successively 8.68 (d, 5.2 Hz, 1H), 8.29 (d, J=4.0 Hz, 1H), 8.23 (t, J=8.0 Hz, 1H), 7.72-7.69 (m, 2H), 7.57 (d, J=5.6 Hz, 1H), 7.47 (s, 1H), 7.39 (s, 1H), 6.71 (s, 1H), 5.78 (dd, J=10.4, 6.8 Hz, 1H), 4.58 (s, 2H), 3.93-3.03 (m, 10H). [M+H⁺] 474.1.

### Example 84

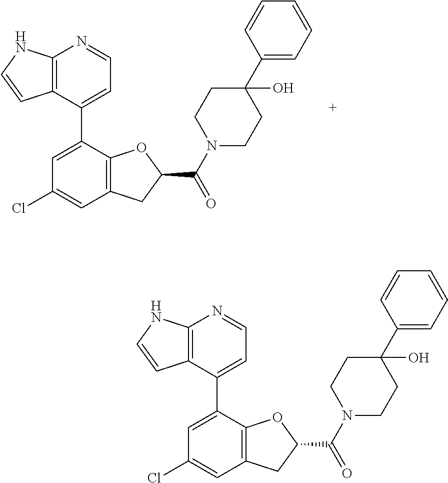 [SMILES: O=C([C@H]1Cc2cc(Cl)cc(-c3ccnc4[nH]ccc34)c2O1)N1CCC(O)(c2ccccc2)CC1 ; O=C([C@@H]1Cc2cc(Cl)cc(-c3ccnc4[nH]ccc34)c2O1)N1CCC(O)(c2ccccc2)CC1]

### (R)-(5-chloro-7-(1H-pyrrolo[2,3-b]pyridin-4-yl)-2,3-dihydrobenzofuran-2-yl)(4-hydroxy-4-phenylpiperidin-1-yl)methanone and (S)-(5-chloro-7-(1H-pyrrolo[2,3-b]pyridin-4-yl)-2,3-dihydrobenzofuran-2-yl)(4-hydroxy-4-phenylpiperidin-1-yl)methanone

A mixture of 5-chloro-7-(1H-pyrrolo[2,3-b]pyridin-4-yl)-2,3-dihydrobenzofuran-2-carboxylic acid (precursor I, step 5) (16 mg, 0.051 mmol) and HATU (38 mg, 0.10 mmol) in DMF (1 mL) was stirred at RT for 30 min. To the reaction mixture was added 4-phenylpiperidin-4-ol (14 mg, 0.087 mmol) and TEA (16 mg, 0.15 mmol). The reaction was stirred at RT for 1 hr. The reaction mixture was diluted with water (1 mL) and extracted with EtOAc, washed with NaHCO₃. The organic layer was concentrated and the residue was purified with column chromatography using 0-8% MeOH/EtOAc to give the title compound (11 mg, 45%). [M+H] 474.1.

### Example 85

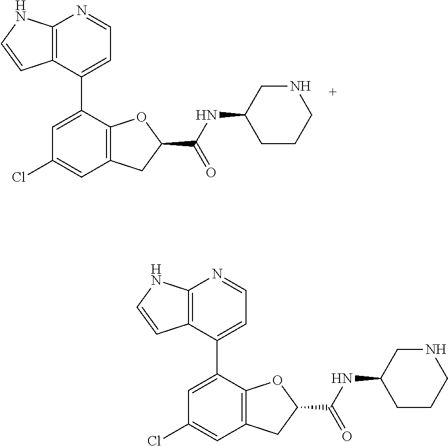 [SMILES: O=C(N[C@@H]1CCCNC1)[C@H]1Cc2cc(Cl)cc(-c3ccnc4[nH]ccc34)c2O1 ; O=C(N[C@@H]1CCCNC1)[C@@H]1Cc2cc(Cl)cc(-c3ccnc4[nH]ccc34)c2O1]

### (R)-5-Chloro-N-((R)-piperidin-3-yl)-7-(1H-pyrrolo[2,3-b]pyridin-4-yl)-2,3-dihydrobenzofuran-2-carboxamide and (S)-5-Chloro-N-((R)-piperidin-3-yl)-7-(1H-pyrrolo[2,3-b]pyridin-4-yl)-2,3-dihydrobenzofuran-2-carboxamide

The title compound was synthesized using precursor I (step 5), general procedure A, C and D using (R)-1-boc-3-aminopiperidine to give the title compound as the HCl salt. ¹H NMR (400 MHz, CD₃OD): δ 8.45 (d, J=6.3 Hz, 1H), 8.34 (t, J=7.1 Hz, 1H), 7.83 (dd, J=6.2, 4.0 Hz, 1H), 7.76 (t, J=3.3 Hz, 1H), 7.56 (s, 1H), 7.47 (d, J=1.1 Hz, 1H), 7.04-6.98 (m, 1H), 5.35 (dd, J=10.3, 7.0 Hz, 1H), 4.15-3.95 (m, 1H), 3.78-3.68 (m, 1H), 3.58-3.36 (m, 2H), 2.98-2.79 (m, 2H), 2.10-1.92 (m, 2H), 1.89-1.71 (m, 1H), 1.71-1.50 (m, 1H); [M+H] 397.0.

### Example 86

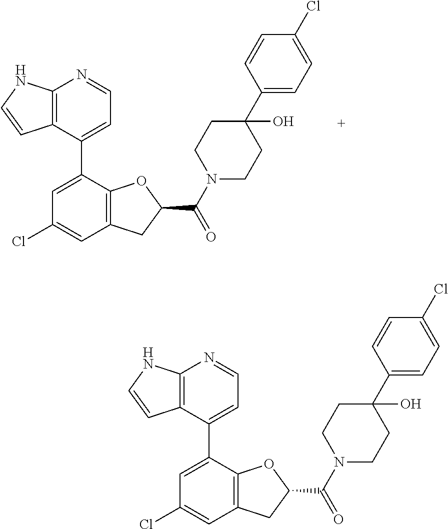 [SMILES: O=C([C@H]1Cc2cc(Cl)cc(-c3ccnc4[nH]ccc34)c2O1)N1CCC(O)(c2ccc(Cl)cc2)CC1 ; O=C([C@@H]1Cc2cc(Cl)cc(-c3ccnc4[nH]ccc34)c2O1)N1CCC(O)(c2ccc(Cl)cc2)CC1]

### (R)-(5-chloro-7-(1H-pyrrolo[2,3-b]pyridin-4-yl)-2,3-dihydrobenzofuran-2-yl)(4-(4-chlorophenyl)-4-hydroxypiperidin-1-yl)methanone and (S)-(5-chloro-7-(1H-pyrrolo[2,3-b]pyridin-4-yl)-2,3-dihydrobenzofuran-2-yl)(4-(4-chlorophenyl)-4-hydroxypiperidin-1-yl)methanone

The example was prepared using precursor I (step 5), general procedures A and C. ¹H NMR (400 MHz, CD₃OD): δ 8.21 (d, J=5.1 Hz, 0.25H), 8.18 (d, J=5.1 Hz, 0.75H), 7.50-7.36 (m, 3H), 7.33-7.30 (m, 2H), 7.24-6.94 (m, 3H), 6.59 (d, J=3.5 Hz, 0.75H), 6.55 (d, J=3.5 Hz, 0.25H), 5.85-5.70 (m, 1H), 4.48-4.31 (m, 1H), 4.06-3.64 (m, 2H), 3.60-3.38 (m, 2H), 3.28-3.03 (m, 2H), 2.09-1.65 (m, 1H), 1.64-1.52 (m, 2H). [M+H] 508.0.

### Example 87

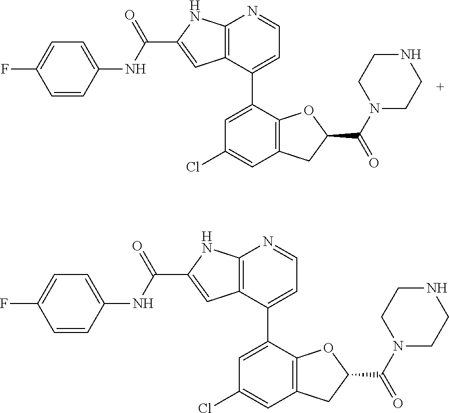 [SMILES: O=C(Nc1ccc(F)cc1)c1cc2c(-c3cc(Cl)cc4c3O[C@@H](C(=O)N3CCNCC3)C4)ccnc2[nH]1 ; O=C(Nc1ccc(F)cc1)c1cc2c(-c3cc(Cl)cc4c3O[C@H](C(=O)N3CCNCC3)C4)ccnc2[nH]1]

### (R)-4-(5-chloro-2-(piperazine-1-carbonyl)-2,3-dihydrobenzofuran-7-yl)-N-(4-fluorophenyl)-1H-pyrrolo[2,3-b]pyridine-2-carboxamide and (S)-4-(5-chloro-2-(piperazine-1-carbonyl)-2,3-dihydrobenzofuran-7-yl)-N-(4-fluorophenyl)-1H-pyrrolo[2,3-b]pyridine-2-carboxamide

The intermediate 4-chloro-1-(phenylsulfonyl)-1H-pyrrolo[2,3-b]pyridine (293 mg, 1 mmol) was dissolved in THF (5 mL) and cooled to −78° C., upon which LDA (0.75 mL, 1.5 eq., 2.0 M in THF) was added and the reaction was stirred for 15 minutes. 4-fluorophenylisocyanate was then added and the reaction warmed up to room temperature. The reaction was quenched with sat. NH₄Cl and a standard workup performed. The crude product was purified with 0-70% EtOAc in hexanes to afford 4-chloro-N-(4-fluorophenyl)-1-(phenylsulfonyl)-1H-pyrrolo[2,3-b]pyridine-2-carboxamide which was used in general procedure B to give intermediate tert-butyl 4-(7-(2-((4-fluorophenyl)carbamoyl)-1-(phenylsulfonyl)-1H-pyrrolo[2,3-b]pyridin-4-yl)-2,3-dihydrobenzofuran-2-carbonyl)piperazine-1-carboxylate which was subsequently stirred with CsCO₃ (5 eq.) in THF/MeOH (1:1) at 35° C. for 1 hour. The crude reaction mixture was filtered and purified with 25-90% EtOAc in hexanes to give intermediate tert-butyl 4-(7-(2-((4-fluorophenyl)carbamoyl)-1H-pyrrolo[2,3-b]pyridin-4-yl)-2,3-dihydrobenzofuran-2-carbonyl)piperazine-1-carboxylate which was then used in general procedure D to give the desired compound as the HCl salt. ¹H NMR (400 MHz, CD₃OD, hydrochloric acid salt): δ 8.56 (d, J=6.2 Hz, 1H), 8.04 (s, 1H), 7.91 (d, J=6.2 Hz, 1H), 7.87-7.71 (m, 2H), 7.64 (d, J=2.1 Hz, 1H), 7.51 (d, J=2.1 Hz, 1H), 7.19-7.04 (m, 2H), 5.98 (dd, J=10.2, 6.8 Hz, 1H), 3.99-3.62 (m, 7H), 3.29-3.14 (m, 3H). [M+H] 520.0.

### Example 88

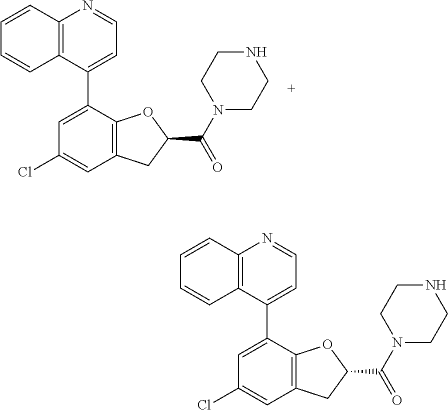 [SMILES: O=C([C@H]1Cc2cc(Cl)cc(-c3ccnc4ccccc34)c2O1)N1CCNCC1 ; O=C([C@@H]1Cc2cc(Cl)cc(-c3ccnc4ccccc34)c2O1)N1CCNCC1]

### (R)-(5-chloro-7-(quinolin-4-yl)-2,3-dihydrobenzofuran-2-yl)(piperazin-1-yl)methanone and (S)-(5-chloro-7-(quinolin-4-yl)-2,3-dihydrobenzofuran-2-yl)(piperazin-1-yl)methanone

The example was prepared from precursor I, general procedures C and D and using quinolin-5-ylboronic acid. ¹H NMR (400 MHz, CD₃OD, hydrochloric acid salt): δ 9.24 (t, J=6.1 Hz, 1H), 8.30 (t, J=8.1 Hz, 2H), 8.25-8.13 (m, 2H), 7.98 (t, J=7.7 Hz, 1H), 7.56 (s, 1H), 7.46 (d, J=1.5 Hz, 1H), 5.83 (t, J=7.3 Hz, 1H), 3.97-3.60 (m, 6H), 3.27-2.88 (m, 4H). [M+H] 394.0.

### Example 89

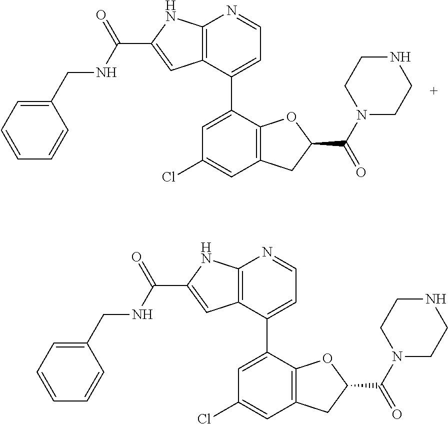 [SMILES: O=C(NCc1ccccc1)c1cc2c(-c3cc(Cl)cc4c3O[C@@H](C(=O)N3CCNCC3)C4)ccnc2[nH]1 ; O=C(NCc1ccccc1)c1cc2c(-c3cc(Cl)cc4c3O[C@H](C(=O)N3CCNCC3)C4)ccnc2[nH]1]

### (R)-N-benzyl-4-(5-chloro-2-(piperazine-1-carbonyl)-2,3-dihydrobenzofuran-7-yl)-1H-pyrrolo[2,3-b]pyridine-2-carboxamide and (S)—N-benzyl-4-(5-chloro-2-(piperazine-1-carbonyl)-2,3-dihydrobenzofuran-7-yl)-1H-pyrrolo[2,3-b]pyridine-2-carboxamide

The intermediate 4-chloro-1-(phenylsulfonyl)-1H-pyrrolo[2,3-b]pyridine-2-carboxylic acid was used in general procedures A and C to give intermediate tert-butyl 4-(7-(2-(benzylcarbamoyl)-1-(phenylsulfonyl)-1H-pyrrolo[2,3-b]pyridin-4-yl)-2,3-dihydrobenzofuran-2-carbonyl)piperazine-1-carboxylate which was subsequently stirred with CsCO₃ (5 eq.) in THF/MeOH (1:1) at 35° C. for 1 hour. The crude reaction mixture was filtered and purified with 25-90% EtOAc in hexanes to give intermediate tert-butyl 4-(7-(2-(benzylcarbamoyl)-1H-pyrrolo[2,3-b]pyridin-4-yl)-2,3-dihydrobenzofuran-2-carbonyl)piperazine-1-carboxylate which was then used in general procedure D to give the desired compound as the HCl salt. ¹H NMR (400 MHz, CD₃OD, hydrochloric acid salt): δ 8.54 (d, J=6.2 Hz, 1H), 7.87-7.85 (m, 2H), 7.59 (d, J=2.2 Hz, 1H), 7.48 (d, J=2.1 Hz, 1H), 7.43-7.32 (m, 4H), 7.29 (dd, J=11.5, 4.4 Hz, 1H), 5.89 (dd, J=10.3, 6.9 Hz, 1H), 4.62 (q, J=15.0 Hz, 2H), 3.89-3.53 (m, 5H), 3.28-3.02 (m, 4H). [M+H] 516.0.

### Example 90

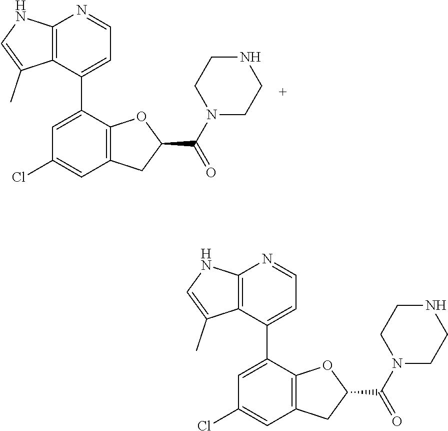 [SMILES: Cc1c[nH]c2nccc(-c3cc(Cl)cc4c3O[C@@H](C(=O)N3CCNCC3)C4)c12 ; Cc1c[nH]c2nccc(-c3cc(Cl)cc4c3O[C@H](C(=O)N3CCNCC3)C4)c12]

### (R)-(5-chloro-7-(3-methyl-1H-pyrrolo[2,3-b]pyridin-4-yl)-2,3-dihydrobenzofuran-2-yl)(piperazin-1-yl)methanone and (S)-(5-chloro-7-(3-methyl-1H-pyrrolo[2,3-b]pyridin-4-yl)-2,3-dihydrobenzofuran-2-yl)(piperazin-1-yl)methanone

The example was prepared from precursor I and 3-methyl-4-(4,4,5,5-tetramethyl-1,3,2-dioxaborolan-2-yl)-1H-pyrrolo[2,3-b]pyridine using general procedures C and D. ¹H NMR (400 MHz, CD₃OD, hydrochloric acid salt): δ 8.40 (d, J=6.1 Hz, 1H), 7.56 (d, J=6.2 Hz, 1H), 7.52 (d, J=1.1 Hz, 1H), 7.48-7.45 (m, 1H), 7.30 (d, J=2.2 Hz, 1H), 5.78 (dd, J=10.0, 6.7 Hz, 1H), 3.93-3.54 (m, 6H), 3.30-2.90 (m, 4H), 2.04 (d, J=1.1 Hz, 3H). [M+H] 397.0.

### Example 91

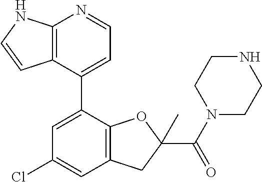 [SMILES: CC1(C(=O)N2CCNCC2)Cc2cc(Cl)cc(-c3ccnc4[nH]ccc34)c2O1]

### (5-chloro-2-methyl-7-(1H-pyrrolo[2,3-b]pyridin-4-yl)-2,3-dihydrobenzofuran-2-yl)(piperazin-1-yl)methanone

The intermediate 7-bromo-5-chloro-2-methyl-2,3-dihydrobenzofuran-2-carboxylic acid was prepared from 2-bromo-4-chloro-phenol and 3-bromo-2-methylprop-1-ene as in steps 1-5 for precursor I. This intermediate was further used with general procedures A, C, and D to give the desired compound as the HCl salt. ¹H NMR (400 MHz, CD₃OD, hydrochloric acid salt) δ 8.51 (d, J=6.4 Hz, 1H), 7.84-7.75 (m, 2H), 7.58 (s, 1H), 7.50 (s, 1H), 6.93 (d, J=3.6 Hz, 1H), 4.13 (d, J=17.0 Hz, 1H), 4.00-3.75 (m, 3H), 3.35-3.28 (m, 2H), 3.26-2.93 (m, 4H), 1.76 (s, 3H). [M+H] 397.0.

### Example 92

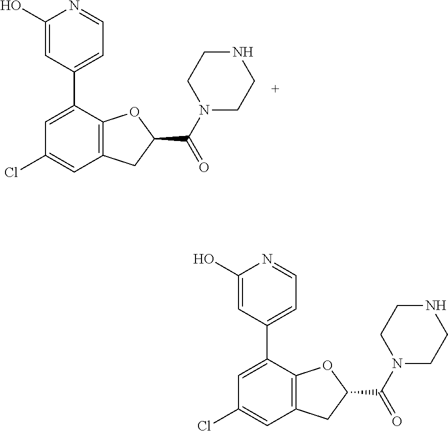 [SMILES: O=C([C@H]1Cc2cc(Cl)cc(-c3ccnc(O)c3)c2O1)N1CCNCC1 ; O=C([C@@H]1Cc2cc(Cl)cc(-c3ccnc(O)c3)c2O1)N1CCNCC1]

### (R)-(5-chloro-7-(2-hydroxypyridin-4-yl)-2,3-dihydrobenzofuran-2-yl)(piperazin-1-yl)methanone and (S)-(5-chloro-7-(2-hydroxypyridin-4-yl)-2,3-dihydrobenzofuran-2-yl)(piperazin-1-yl)methanone

The intermediate tert-butyl 4-(5-chloro-7-(2-methoxypyridin-4-yl)-2,3-dihydrobenzofuran-2-carbonyl)piperazine-1-carboxylate was prepared using 2-methoxy-4-pyridine boronic acid in general procedure A and purified using 0-20% EtOAc in CH₂Cl₂. The resulting material (40 mg, 0.13 mmol) was dissolved in CH₂Cl₂ (5 mL) and cooled at 0° C. at which time BBr₃ (3 mL, 1N in CH₂Cl₂, 20 eq.) was added and the reaction heated at 35° C. for 2 days. The reaction was quenched with 1 mL of MeOH at 0° C., concentrated in vacuo, and purified using 30% MeOH in CH₂Cl₂ to give the desired compound. ¹H NMR (400 MHz, CD₃OD): δ 7.60-7.48 (m, 1H), 7.39 (s, 1H), 7.35 (s, 1H), 6.98-6.93 (m, 1H), 6.84-6.75 (m, 1H), 5.86-5.74 (m, 1H), 4.62 (s, 2H), 4.12-3.42 (m, 4H), 3.27-3.04 (m, 4H). [M+H] 360.1.

### Example 93

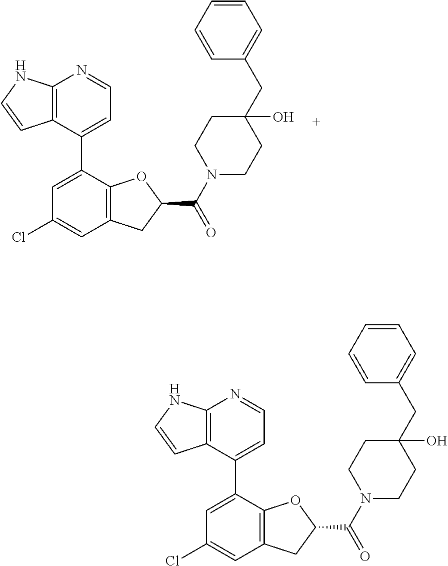 [SMILES: O=C([C@H]1Cc2cc(Cl)cc(-c3ccnc4[nH]ccc34)c2O1)N1CCC(O)(Cc2ccccc2)CC1 ; O=C([C@@H]1Cc2cc(Cl)cc(-c3ccnc4[nH]ccc34)c2O1)N1CCC(O)(Cc2ccccc2)CC1]

### (R)-(4-benzyl-4-hydroxypiperidin-1-yl)(5-chloro-7-(1H-pyrrolo[2,3-b]pyridin-4-yl)-2,3-dihydrobenzofuran-2-yl)methanone and (S)-(4-benzyl-4-hydroxypiperidin-1-yl)(5-chloro-7-(1H-pyrrolo[2,3-b]pyridin-4-yl)-2,3-dihydrobenzofuran-2-yl)methanone

The example was prepared using 7-bromo-5-chloro-2,3-dihydrobenzofuran-2-carboxylic acid (0.3 mmol) from precursor I using general procedure A and employing HATU coupling conditions and purification with 0-8% MeOH in EtOAc. The resulting material was used in general procedure C give the desire compound. ¹H NMR (400 MHz, (CD₃)₂SO): δ 8.26 (dd, J=15.9, 4.9 Hz, 1H), 7.53-7.47 (m, 1H), 7.38 (s, 2H), 7.29-7.11 (m, 5H), 7.02 (d, J=6.8 Hz, 1H), 6.54 (s, 1H), 5.76 (ddd, J=9.8, 6.5, 3.1 Hz, 1H), 4.47 (d, J=20.4 Hz, 1H), 4.16-3.98 (m, 1H), 3.77-3.62 (m, 1H), 3.57-3.19 (m, 4H), 3.02-2.85 (m, 1H), 1.55-1.32 (m, 2H), 1.30-1.13 (m, 2H). [M+H] 488.1.

### Example 94

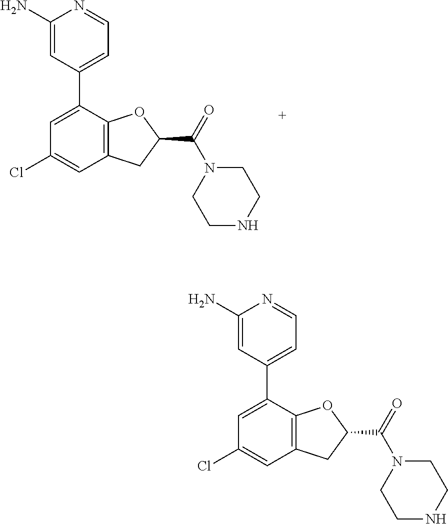 [SMILES: Nc1cc(-c2cc(Cl)cc3c2O[C@@H](C(=O)N2CCNCC2)C3)ccn1 ; Nc1cc(-c2cc(Cl)cc3c2O[C@H](C(=O)N2CCNCC2)C3)ccn1]

### (7-(2-Aminopyridin-4-yl)-5-chloro-2,3-dihydrobenzofuran-2-yl)(piperazin-1-yl)methanone

The example was prepared as the HCl salt using precursor I, general procedures C and D with 2-aminopyridine-4-boronic acid to give the desired compound as the HCl salt. ¹H NMR (400 MHz, CD₃OD, hydrochloric acid salt): δ ppm 7.87 (d, J=6.9 Hz, 1H), 7.50 (d, J=2.1 Hz, 1H), 7.45-7.39 (m, 2H), 7.33 (dd, J=6.9, 1.7 Hz, 1H), 5.88 (dd, J=9.9, 6.6 Hz, 1H), 4.04-3.75 (m, 4H), 3.65-3.54 (m, 2H), 3.39-3.26 (m, 4H). [M+H] 359.1.

### Examples 95 and 96

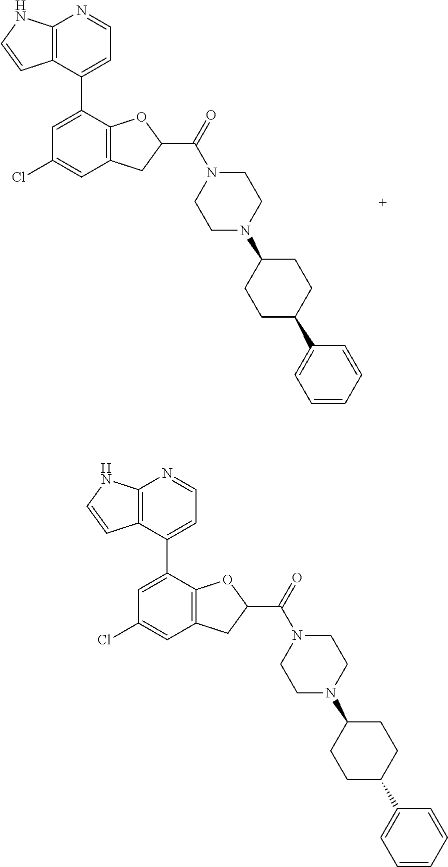 [SMILES: O=C(C1Cc2cc(Cl)cc(-c3ccnc4[nH]ccc34)c2O1)N1CCN([C@H]2CC[C@@H](c3ccccc3)CC2)CC1 ; O=C(C1Cc2cc(Cl)cc(-c3ccnc4[nH]ccc34)c2O1)N1CCN([C@H]2CC[C@H](c3ccccc3)CC2)CC1]

### Step 1: tert-butyl 4-(4-phenylcyclohexyl)piperazine-1-carboxylate

A mixture of 4-phenylcyclohexanone (700 mg, 4 mmol), N-Boc-piperazine (748 mg, 4 mmol), and sodium triacetoxyborohydride (3.4 g, 16 mmol) in 1,2-DCE (20 mL) was stirred at RT for 48 hrs. The reaction mixture was concentrated, diluted with water, and extracted with EtOAc. The organic layer was concentrated and the residue was purified with column chromatography using 10-50% EtOAc/hexanes to afford tert-butyl 4-(4-phenylcyclohexyl)piperazine-1-carboxylate (190 mg, 14%) as a mixture of stereoisomers.

### Step 2: 1-(4-phenylcyclohexyl)piperazine

The tert-butyl 4-(4-phenylcyclohexyl)piperazine-1-carboxylate (190 mg, 0.55 mmol) was dissolved in DCM (2 mL) and TFA (1 mL) was then added. The reaction was stirred at RT for 1 hr, then concentrated, and dried to afford 1-(4-phenylcyclohexyl)piperazine (124 mg, 92%).

### Step 3: 5-chloro-7-(1H-pyrrolo[2,3-b]pyridin-4-yl)-2,3-dihydrobenzofuran-2-carboxylic Acid

A mixture of 7-bromo-5-chloro-2,3-dihydrobenzofuran-2-carboxylic acid (329 mg, 1.19 mmol), 4-(4,4,5,5-tetramethyl-1,3,2-dioxaborolan-2-yl)-1H-pyrrolo[2,3-b]pyridine (434 mg, 1.78 mmol), Pd(dppf)Cl₂) (87 mg, 0.12 mmol), Na₂CO₃ (378 mg, 3.57 mmol) in DMF (5 mL), and water (0.5 mL) was heated at 95° C. and stirred for overnight. The reaction mixture was cooled to room temperature, diluted with water and extracted with EtOAc. The impurities stayed in the first organic extract. Then the aqueous layer was neutralized and extracted again with EtOAc (3×). The organic layer was washed with water, brine, dried over MgSO₄, concentrated. The crude 5-chloro-7-(1H-pyrrolo[2,3-b]pyridin-4-yl)-2,3-dihydrobenzofuran-2-carboxylic acid was precipitated as a brown solid (219 mg, 58%).

### Step 4: (5-chloro-7-(1H-pyrrolo[2,3-b]pyridin-4-yl)-2,3-dihydrobenzofuran-2-yl)(4-((1s,4s)-4-phenylcyclohexyl)piperazin-1-yl)methanone and (5-chloro-7-(1H-pyrrolo[2,3-b]pyridin-4-yl)-2,3-dihydrobenzofuran-2-yl)(4-((1r,4r)-4-phenylcyclohexyl)piperazin-1-yl)methanone

A mixture of crude 5-chloro-7-(1H-pyrrolo[2,3-b]pyridin-4-yl)-2,3-dihydrobenzofuran-2-carboxylic acid (60 mg, 0.19 mmol) and HATU (144 mg, 0.38 mmol) in DMF (2 mL) was stirred at RT for 30 min. To the reaction mixture was added 1-(4-phenylcyclohexyl)piperazine (69 mg, 0.29 mmol) and TEA (30 mg, 0.3 mmol). The reaction was stirred at RT for 1 hr. The reaction mixture was diluted with water (1 mL) and extracted with EtOAc, washed with NaHCO₃. The organic layer was concentrated and the residue was purified with column chromatography using 0-6% MeOH/EtOAc to afford (5-chloro-7-(1H-pyrrolo[2,3-b]pyridin-4-yl)-2,3-dihydrobenzofuran-2-yl)(4-((1s,4s)-4-phenylcyclohexyl)piperazin-1-yl)methanone (10.1 mg) and (5-chloro-7-(1H-pyrrolo[2,3-b]pyridin-4-yl)-2,3-dihydrobenzofuran-2-yl)(4-((1r,4r)-4-phenylcyclohexyl)piperazin-1-yl)methanone (7.2 mg, 17% total). ¹H NMR (400 MHz, CD₃OD, hydrochloric acid salt): δ ppm 11.74 (s, 1H), 8.23 (d, J=5.0 Hz, 1H), 7.53-7.45 (m, 1H), 7.37 (s, 2H), 7.32-7.18 (m, 5H), 7.18-7.10 (m, 1H), 6.57-6.51 (m, 1H), 5.78 (s, 1H), 3.69-3.18 (m, 8H), 2.71-2.08 (m, J=3.6, 1.8 Hz, 4H), 1.95-1.74 (m, 4H), 1.57-1.40 (m, 4H). [M+H] 5410.

### Example 97

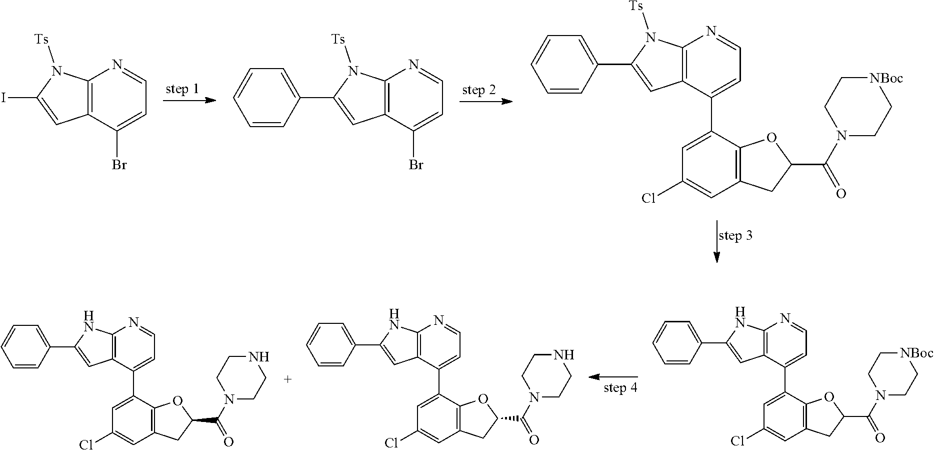 [SMILES: Cc1ccc(S(=O)(=O)n2c(I)cc3c(Br)ccnc32)cc1 ; Cc1ccc(S(=O)(=O)n2c(-c3ccccc3)cc3c(Br)ccnc32)cc1 ; Cc1ccc(S(=O)(=O)n2c(-c3ccccc3)cc3c(-c4cc(Cl)cc5c4OC(C(=O)N4CCN(C(=O)OC(C)(C)C)CC4)C5)ccnc32)cc1 ; O=C([C@H]1Cc2cc(Cl)cc(-c3ccnc4[nH]c(-c5ccccc5)cc34)c2O1)N1CCNCC1 ; O=C([C@@H]1Cc2cc(Cl)cc(-c3ccnc4[nH]c(-c5ccccc5)cc34)c2O1)N1CCNCC1 ; CC(C)(C)OC(=O)N1CCN(C(=O)C2Cc3cc(Cl)cc(-c4ccnc5[nH]c(-c6ccccc6)cc45)c3O2)CC1]

### Step 1: 4-bromo-2-phenyl-1-tosyl-1H-pyrrolo[2,3-b]pyridine

A mixture of 4-bromo-2-iodo-1-tosyl-1H-pyrrolo[2,3-b]pyridine (200 mg, 0.42 mmol), phenyl-boronic acid (51 mg, 1.0 eq), Pd(PPh3)4 (20 mg, 0.05 eq) and K₂CO₃ (173 mg, 3 eq) was dissolved in DMF (3 mL) and H₂O (1 mL). The reaction was heated to 100° C. for 3 hr. The reaction mixture was diluted with EtOAc and washed with H₂O, NaHCO₃ solution and brine. The crude reaction mixture was concentrated and purified by flash column chromatography (silica gel, 0-30% EtOAc in hexanes) to afford 4-bromo-2-phenyl-1-tosyl-1H-pyrrolo[2,3-b]pyridine (25 mg, 14% yield).

### Step 2: tert-butyl 4-(5-chloro-7-(2-phenyl-1-tosyl-1H-pyrrolo[2,3-b]pyridin-4-yl)-2,3-dihydrobenzofuran-2-carbonyl)piperazine-1-carboxylate

A mixture of tert-butyl 4-(5-chloro-7-(tributylstannyl)-2,3-dihydrobenzofuran-2-carbonyl)piperazine-1-carboxylate (34 mg, 0.052 mmol), 4-bromo-2-phenyl-1-tosyl-1H-pyrrolo[2,3-b]pyridine (22 mg, 0.052 mmol), and tetrakis(triphenylphosphine)palladium(0) (7 mg, 0.006 mmol) in DMF (1.5 mL) was heated to 120° C. and stirred for 6 hr. The reaction mixture was cooled to room temperature, diluted with water and extracted with EtOAc. The organic layer was washed with water, brine, dried over MgSO₄, concentrated. The crude product was purified by chromatography (0-7% MeOH/EtOAc) to afford tert-butyl 4-(5-chloro-7-(2-phenyl-1-tosyl-1H-pyrrolo[2,3-b]pyridin-4-yl)-2,3-dihydrobenzofuran-2-carbonyl)piperazine-1-carboxylate (14 mg, 38%).

### Step 3: tert-butyl 4-(5-chloro-7-(2-phenyl-1H-pyrrolo[2,3-b]pyridin-4-yl)-2,3-dihydrobenzofuran-2-carbonyl)piperazine-1-carboxylate

tert-butyl 4-(5-chloro-7-(2-phenyl-1-tosyl-1H-pyrrolo[2,3-b]pyridin-4-yl)-2,3-dihydrobenzofuran-2-carbonyl)piperazine-1-carboxylate (14 mg, 0.02 mmol) was dissolved in THF/MeOH (1:1) (2 mL) and Cs₂CO₃ (25 mg, 0.078 mmol) was added. The mixture was stirred at 50° C. for 4 hr. The reaction mixture was filtered through silica gel and washed with EtOAc. The filtrate was concentrated to afford tert-butyl 4-(5-chloro-7-(2-phenyl-1H-pyrrolo[2,3-b]pyridin-4-yl)-2,3-dihydrobenzofuran-2-carbonyl)piperazine-1-carboxylate (8 mg, 71%).

### Step 4

(R)-(5-chloro-7-(2-phenyl-1H-pyrrolo[2,3-b]pyridin-4-yl)-2,3-dihydrobenzofuran-2-yl)(piperazin-1-yl)methanone and (S)-(5-chloro-7-(2-phenyl-1H-pyrrolo[2,3-b]pyridin-4-yl)-2,3-dihydrobenzofuran-2-yl)(piperazin-1-yl)methanone were prepared from 4-(5-chloro-7-(2-phenyl-1H-pyrrolo[2,3-b]pyridin-4-yl)-2,3-dihydrobenzofuran-2-carbonyl)piperazine-1-carboxylate according to general procedure E. ¹H NMR (400 MHz, CD₃OD, HCl): δ ppm 1H NMR (400 MHz, DMSO-d6): δ 12.25 (s, 1H), 8.29-8.21 (m, 1H), 7.98 (d, J=7.6 Hz, 2H), 7.51-7.30 (m, 5H), 7.26 (d, J=4.8 Hz, 1H), 7.19 (s, 1H), 5.92-5.82 (m, 1H), 3.82-3.35 (m, 4H), 3.19-2.83 (m, J=52.8 Hz, 4H), 2.44-2.37 (m, 2H). [M+H] 459.1.

### Example 98

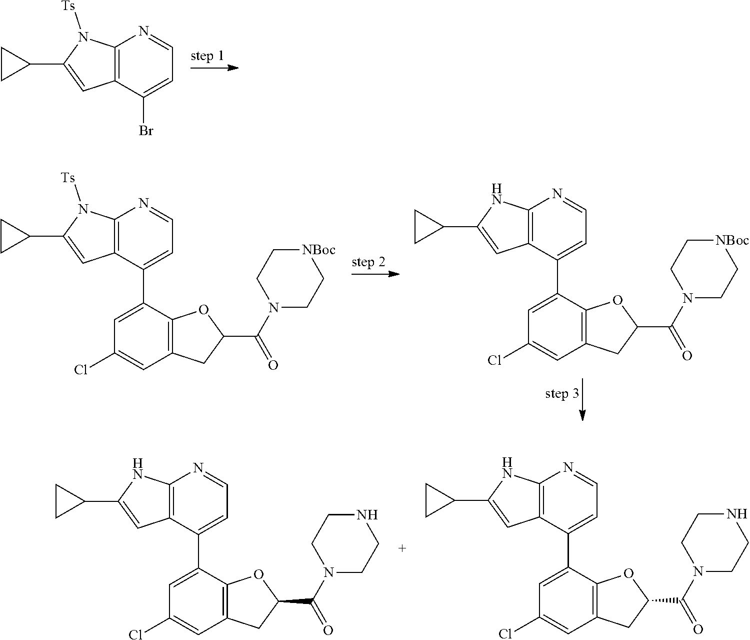 [SMILES: Cc1ccc(S(=O)(=O)n2c(C3CC3)cc3c(Br)ccnc32)cc1 ; Cc1ccc(S(=O)(=O)n2c(C3CC3)cc3c(-c4cc(Cl)cc5c4OC(C(=O)N4CCN(C(=O)OC(C)(C)C)CC4)C5)ccnc32)cc1 ; CC(C)(C)OC(=O)N1CCN(C(=O)C2Cc3cc(Cl)cc(-c4ccnc5[nH]c(C6CC6)cc45)c3O2)CC1 ; O=C([C@H]1Cc2cc(Cl)cc(-c3ccnc4[nH]c(C5CC5)cc34)c2O1)N1CCNCC1 ; O=C([C@@H]1Cc2cc(Cl)cc(-c3ccnc4[nH]c(C5CC5)cc34)c2O1)N1CCNCC1]

### Step 1: tert-butyl 4-(5-chloro-7-(2-cyclopropyl-1-tosyl-1H-pyrrolo[2,3-b]pyridin-4-yl)-2,3-dihydrobenzofuran-2-carbonyl)piperazine-1-carboxylate

A mixture of tert-butyl 4-(5-chloro-7-(tributylstannyl)-2,3-dihydrobenzofuran-2-carbonyl)piperazine-1-carboxylate (50 mg, 0.076 mmol), 4-bromo-2-cyclopropyl-1-tosyl-1H-pyrrolo[2,3-b]pyridine (55 mg, 0.16 mmol), and tetrakis(triphenylphosphine)palladium(0) (7 mg, 0.012 mmol) in DMF (2 mL) was heated at 140° C. and stirred for 30 hr. The reaction mixture was cooled to room temperature, diluted with water and extracted with EtOAc. The organic layer was washed with water, and concentrated. The crude product was purified by chromatography (3-7% MeOH/EtOAc) to afford tert-butyl 4-(5-chloro-7-(2-cyclopropyl-1-tosyl-1H-pyrrolo[2,3-b]pyridin-4-yl)-2,3-dihydrobenzofuran-2-carbonyl)piperazine-1-carboxylate (11 mg, 21%).

### Step 2: tert-butyl 4-(5-chloro-7-(2-cyclopropyl-1H-pyrrolo[2,3-b]pyridin-4-yl)-2,3-dihydrobenzofuran-2-carbonyl)piperazine-1-carboxylate

tert-butyl 4-(5-chloro-7-(2-cyclopropyl-1-tosyl-1H-pyrrolo[2,3-b]pyridin-4-yl)-2,3-dihydrobenzofuran-2-carbonyl)piperazine-1-carboxylate (11 mg, 0.016 mmol) was dissolved in THF/MeOH (1:1) (2 mL) and Cs₂CO₃ was added (21 mg, 0.065 mmol). The reaction was stirred at 50° C. for 24 hr. The reaction mixture was filtered through silica gel, and washed with EtOAc. The filtrate was concentrated and purified by chromatography (3-7% MeOH/EtOAc) to afford tert-butyl 4-(5-chloro-7-(2-cyclopropyl-1H-pyrrolo[2,3-b]pyridin-4-yl)-2,3-dihydrobenzofuran-2-carbonyl)piperazine-1-carboxylate (5.5 mg, 71%).

### Step 3

(5-chloro-7-(2-cyclopropyl-1H-pyrrolo[2,3-b]pyridin-4-yl)-2,3-dihydrobenzofuran-2-yl)(piperazin-1-yl)methanone was prepared from tert-butyl 4-(5-chloro-7-(2-cyclopropyl-1H-pyrrolo[2,3-b]pyridin-4-yl)-2,3-dihydrobenzofuran-2-carbonyl)piperazine-1-carboxylate according to general procedure E. [M+H^{+]} 423.0.

### Example 99

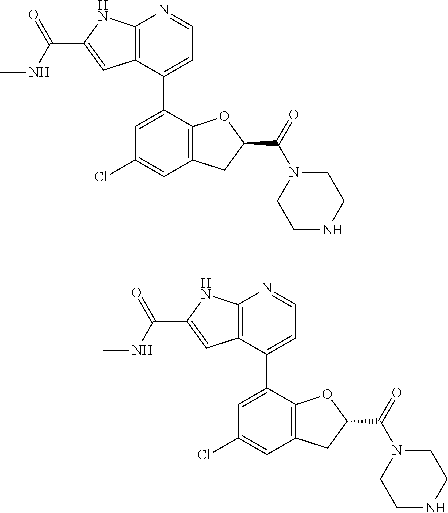 [SMILES: CNC(=O)c1cc2c(-c3cc(Cl)cc4c3O[C@@H](C(=O)N3CCNCC3)C4)ccnc2[nH]1 ; CNC(=O)c1cc2c(-c3cc(Cl)cc4c3O[C@H](C(=O)N3CCNCC3)C4)ccnc2[nH]1]

### Step 1: 1-((4-bromophenyl)sulfonyl)-4-chloro-N-methyl-1H-pyrrolo[2,3-b]pyridine-2-carboxamide

1-((4-bromophenyl)sulfonyl)-4-chloro-1H-pyrrolo[2,3-b]pyridine-2-carboxylic acid (50.5 mg, 0.15 mmol), 1-methylimidazole (24.6 mg, 2.0 eq), and methylamine (2M in THF, 0.15 mL, 2.0 eq) were combined in EtOAc (2 mL). 1-propanephosphonic acid cyclic anhydride (71.6 mg, 1.5 eq) was added and the reaction was stirred for 30 min. The mixture was diluted with 1M sodium hydroxide (2 mL) and extracted with EtOAc (3×5 mL). The organic layers were dried over sodium sulfate, filtered, and concentrated in vacuo. The crude residue was purified by flash column chromatography (50-100% EtOAc in hexanes) to afford 1-((4-bromophenyl)sulfonyl)-4-chloro-N-methyl-1H-pyrrolo[2,3-b]pyridine-2-carboxamide which was used directly in the following step.

### Step 2: tert-butyl 4-(7-(1-((4-bromophenyl)sulfonyl)-2-(methylcarbamoyl)-1H-pyrrolo[2,3-b]pyridin-4-yl)-5-chloro-2,3-dihydrobenzofuran-2-carbonyl)piperazine-1-carboxylate

1-(benzenesulfonyl)-4-chloro-N-methyl-pyrrolo[2,3-b]pyridine-2-carboxamide (52.5 mg, 0.15 mmol, 1.5 eq), tert-butyl 4-(5-chloro-7-tributylstannyl-2,3-dihydrobenzofuran-2-carbonyl)piperazine-1-carboxylate (65.6 mg, 0.1 mmol), and tetrakis(triphenylphosphine)palladium(0) (11.6 mg, 0.1 eq) in 1,4-dioxane (0.5 mL) were mixed and sparged with Ar gas for 5 min. The mixture was heated with stirring at 170° C. in a sealed microwave vial for 4 h before being diluted with EtOAc and filtered over celite. The mixture was concentrated to afford tert-butyl 4-(7-(1-((4-bromophenyl)sulfonyl)-2-(methylcarbamoyl)-1H-pyrrolo[2,3-b]pyridin-4-yl)-5-chloro-2,3-dihydrobenzofuran-2-carbonyl)piperazine-1-carboxylate which was used directly in the following step.

### Step 3: tert-butyl 4-(5-chloro-7-(2-(methylcarbamoyl)-1H-pyrrolo[2,3-b]pyridin-4-yl)-2,3-dihydrobenzofuran-2-carbonyl)piperazine-1-carboxylate

tert-butyl 4-(7-(1-((4-bromophenyl)sulfonyl)-2-(methylcarbamoyl)-1H-pyrrolo[2,3-b]pyridin-4-yl)-5-chloro-2,3-dihydrobenzofuran-2-carbonyl)piperazine-1-carboxylate (12 mg, 0.016 mmol) was dissolved in THF/MeOH (1:1) (2 mL) and Cs₂CO₃ was added (21 mg, 0.065 mmol). The reaction was stirred at 50° C. for 24 hr. The reaction mixture was filtered through silica gel, and washed with EtOAc. The filtrate was concentrated and purified by chromatography (3-7% MeOH/EtOAc) to afford tert-butyl 4-(5-chloro-7-(2-(methylcarbamoyl)-1H-pyrrolo[2,3-b]pyridin-4-yl)-2,3-dihydrobenzofuran-2-carbonyl)piperazine-1-carboxylate, which was used directly in the following step.

### Step 4

4-(5-chloro-2-(piperazine-1-carbonyl)-2,3-dihydrobenzofuran-7-yl)-N-methyl-1H-pyrrolo[2,3-b]pyridine-2-carboxamide was prepared from tert-butyl 4-(5-chloro-7-(2-(methylcarbamoyl)-1H-pyrrolo[2,3-b]pyridin-4-yl)-2,3-dihydrobenzofuran-2-carbonyl)piperazine-1-carboxylate according to general procedure D. ¹H NMR (400 MHz, CD₃OD): δ 8.52 (dd, J=6.1, 0.9 Hz, 1H), 7.85 (dd, J=6.1, 0.9 Hz, 1H), 7.64 (d, J=0.9 Hz, 1H), 7.60 (dd, J=2.2, 0.9 Hz, 1H), 7.50 (dd, J=2.2, 0.9 Hz, 1H), 5.91 (t, J=8.5 Hz, 1H), 3.95-3.67 (m, 7H), 3.18 (dd, J=54.6, 13.2 Hz, 3H), 2.97 (d, J=0.9 Hz, 3H); [M+H^{+]} 440.0.

### Example 100

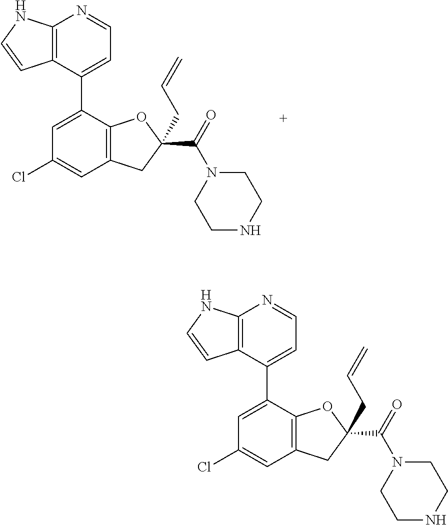 [SMILES: C=CC[C@]1(C(=O)N2CCNCC2)Cc2cc(Cl)cc(-c3ccnc4[nH]ccc34)c2O1 ; C=CC[C@@]1(C(=O)N2CCNCC2)Cc2cc(Cl)cc(-c3ccnc4[nH]ccc34)c2O1]

### Step 1: tert-butyl 4-(2-allyl-7-bromo-5-chloro-2,3-dihydrobenzofuran-2-carbonyl)piperazine-1-carboxylate

To tert-butyl 4-(7-bromo-5-chloro-2,3-dihydrobenzofuran-2-carbonyl)piperazine-1-carboxylate (446 mg, 1.0 mmol) in THF (5 mL) at −78° C. is added lithium hexamethyldisilazide solution (1M in THF, 1.05 mL, 1.05 eq). The reaction mixture was stirred for 15 min before the addition of allyl bromide (257 mg, 1.5 eq). The reaction was stirred for 1 h before being quenched with acetic acid (0.25 mL). The reaction mixture is diluted with water (25 mL) and EtOAc (25 mL) and the organic layer is dried, filtered, and concentrated. The crude residue is purified by flash column chromatography (0-25% EtOAc in hexanes) to afford tert-butyl 4-(2-allyl-7-bromo-5-chloro-2,3-dihydrobenzofuran-2-carbonyl)piperazine-1-carboxylate.

### Step 2: (2-allyl-5-chloro-7-(1H-pyrrolo[2,3-b]pyridin-4-yl)-2,3-dihydrobenzofuran-2-yl)(piperazin-1-yl)methanone was prepared from tert-butyl 4-(2-allyl-7-bromo-5-chloro-2,3-dihydrobenzofuran-2-carbonyl)piperazine-1-carboxylate using general procedures C and D. ¹H NMR (400 MHz, CD₃OD): δ 8.49 (d, J=6.2 Hz, 1H), 7.78 (d, J=3.6 Hz, 1H), 7.76 (d, J=6.1 Hz, 1H), 7.57 (d, J=2.2 Hz, 1H), 7.48-7.46 (m, 1H), 6.92 (d, J=3.6 Hz, 1H), 5.85-5.71 (m, 1H), 5.23 (dd, J=31.9, 12.7 Hz, 2H), 4.04 (d, J=17.1 Hz, 1H), 3.89 (br s, 4H), 3.47 (d, J=17.1 Hz, 1H), 3.13 (br m, 4H), 2.87 (dd, J=14.2, 7.5 Hz, 1H), 2.80-2.70 (m, 1H); [M+H⁺] 423.1

### Example 101

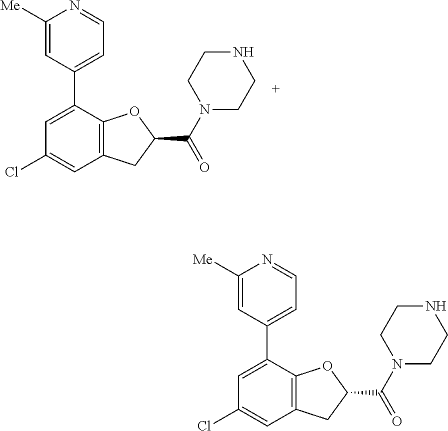 [SMILES: Cc1cc(-c2cc(Cl)cc3c2O[C@@H](C(=O)N2CCNCC2)C3)ccn1 ; Cc1cc(-c2cc(Cl)cc3c2O[C@H](C(=O)N2CCNCC2)C3)ccn1]

### (5-Chloro-7-(2-methylpyridin-4-yl)-2,3-dihydrobenzofuran-2-yl)(piperazin-1-yl)methanone

The example was prepared as the HCl salt using precursor I, general procedures C and D with 2-methylpyridine-4-boronic acid to give the desired compound as HCl salt. ¹H NMR (400 MHz, CD₃OD, hydrochloric acid salt): δ 8.67 (d, J=6.3 Hz, 1H), 8.34-8.28 (m, 2H), 7.71-7.68 (m, 1H), 7.49-7.46 (m, 1H), 5.93 (dd, J=10.2, 6.2 Hz, 1H), 4.05-3.75 (m, 4H), 3.75-3.52 (m, 2H), 3.42-3.20 (m, 4H), 2.81 (s, 3H). [M+H⁺] 358.0.

### Example 102

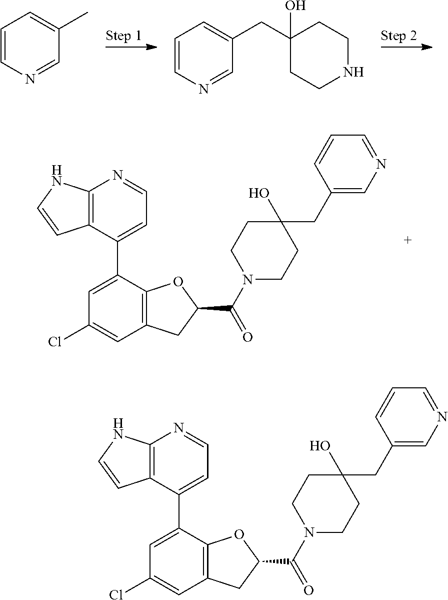

### Step 1: 4-(pyridin-3-ylmethyl)piperidin-4-ol

To a cooled solution of 3-methyl-pyridine (930 mg, 10 mmol) in THF (20 mL) at −40° C. was added LDA (1M, 11 mL, 11 mmol) dropwise. After 30 min, 4-Oxo-piperidine-1-carboxylic acid tert-butyl ester (2.00 g, 1 eq) in THF (5 mL) was added dropwise and warmed to 0° C. The reaction mixture was quenched with NH₄Cl solution and extracted with EtOAc (30 mL) and purified by column (EtOAc/MeOH, 10/1) to give a yellow oil (150 mg). This material was then dissolved in DCM and HCl (4M in dioxane, 5 eq) was added. After stirring for 3 hr., volatiles were removed under reduced pressure. The resulting residue was dissolved in MeOH/DCM (20/80, 2 mL) and filtered through a PL-HCO3 MP SPE tube to give the freebase of 4-(pyridin-3-ylmethyl)piperidin-4-ol.

### Step 2: (R)-(5-chloro-7-(1H-pyrrolo[2,3-b]pyridin-4-yl)-2,3-dihydrobenzofuran-2-yl)(4-hydroxy-4-(pyridin-3-ylmethyl)piperidin-1-yl)methanone and (R)-(5-chloro-7-(1H-pyrrolo[2,3-b]pyridin-4-yl)-2,3-dihydrobenzofuran-2-yl)(4-hydroxy-4-(pyridin-3-ylmethyl)piperidin-1-yl)methanone

The example was prepared using precursor I, general procedures A, C using 4-(pyridin-3-ylmethyl)piperidin-4-ol and purified by flash column chromatography eluting with 4 to 9% MeOH in EtOAc as the eluent followed to give the desired compound as the free-base. ¹H NMR (400 MHz, DMSO-D₆): δ 11.75 (d, J=18.4 Hz, 1H), 8.51-8.29 (m, 2H), 8.24 (dd, J=15.7, 4.9 Hz, 1H), 7.68-7.13 (m, 6H), 6.62-6.47 (m, 1H), 5.80-5.69 (m, 1H), 4.60-4.53 (m, 1H), 3.82-3.40 (m, 4H), 2.78-2.57 (m, 1H), 1.55-1.11 (m, 6H); [M+H] 489.

### Example 103

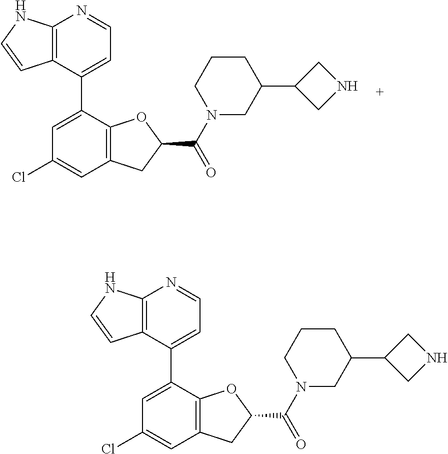 [SMILES: O=C([C@H]1Cc2cc(Cl)cc(-c3ccnc4[nH]ccc34)c2O1)N1CCCC(C2CNC2)C1 ; O=C([C@@H]1Cc2cc(Cl)cc(-c3ccnc4[nH]ccc34)c2O1)N1CCCC(C2CNC2)C1]

### (3-(azetidin-3-yl)piperidin-1-yl)((R)-5-chloro-7-(1H-pyrrolo[2,3-b]pyridin-4-yl)-2,3-dihydrobenzofuran-2-yl)methanone and (3-(azetidin-3-yl)piperidin-1-yl)((S)-5-chloro-7-(1H-pyrrolo[2,3-b]pyridin-4-yl)-2,3-dihydrobenzofuran-2-yl)methanone

The example was prepared using precursor I (step 5), general procedure A, C and D using tert-butyl 3-(3-piperidyl)azetidine-1-carboxylate to give the desired compound as the HCl salt. ¹H NMR (400 MHz, CD₃OD, hydrochloric acid salt): δ 8.52-8.37 (m, 1H), 7.93-7.80 (m, 1H), 7.75 (dd, J=7.3, 3.1 Hz, 1H), 7.56 (d, J=2.1 Hz, 1H), 7.48 (s, 1H), 7.07 (d, J=3.6 Hz, 1H), 5.87 (dd, J=10.0, 6.6 Hz, 1H), 4.20-3.68 (m, 7H), 3.64-3.41 (m, 2H), 3.28-3.10 (m, 1H), 2.92-2.63 (m, 2H), 2.07-1.07 (m, 4H); [M+H] 437.0

### Example 104

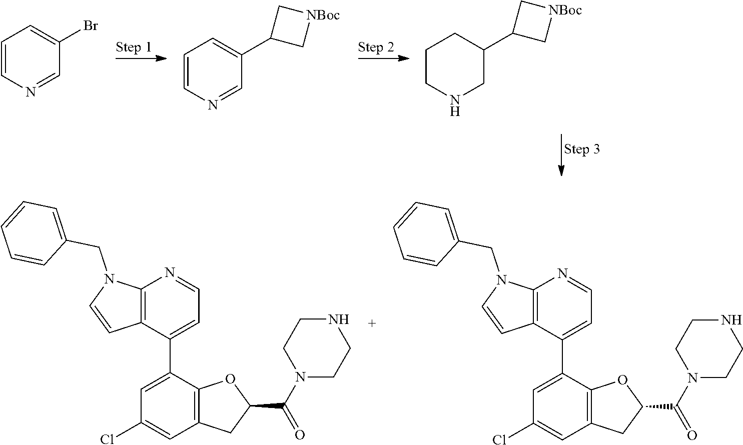 [SMILES: Brc1cccnc1 ; CC(C)(C)OC(=O)N1CC(c2cccnc2)C1 ; CC(C)(C)OC(=O)N1CC(C2CCCNC2)C1 ; O=C([C@H]1Cc2cc(Cl)cc(-c3ccnc4c3ccn4Cc3ccccc3)c2O1)N1CCNCC1 ; O=C([C@@H]1Cc2cc(Cl)cc(-c3ccnc4c3ccn4Cc3ccccc3)c2O1)N1CCNCC1]

### Step 1: tert-butyl 3-(pyridin-3-yl)azetidine-1-carboxylate

A solution of isopropylmagnesium chloride (2.0 M in THF, 371 mL, 742 mmol) was slowly added into a solution of the 3-bromopyridine (111.6 g, 706 mmol) and flame-dried LiCl (706 mmo. 29.9 gl) in THF (440 mL) at 5° C. over 20 min. The resulting mixture was stirred at the low temperature for 30 min, and then at room temperature until all LiCl went into solution (3-4 hrs). The above Grignard reagent mixture was cooled at 0° C. again. A solution of the iodocyclobutane (100.2 g, 354 mmol) and CoCl₂ (2.29 g, 17.6 mmol) in THF (440 mL) was slowly added into the above cold solution through a dropping funnel over 30 min. The reaction mixture was allowed to stir at room temperature for 1 hour. The reaction was quenched with a saturated NH₄Cl solution, and then diluted with water. The product was extracted with EtOAc 2 times. The combined organic layers were washed with brine and then dried over sodium sulfate. The crude product was purified by silica chromatography (5% methanol in DCM) to afford a pure tert-butyl 3-(pyridin-3-yl)azetidine-1-carboxylate (63.55 g, 77%) as a brownish liquid.

### Step 2: tert-butyl 3-(piperidin-3-yl)azetidine-1-carboxylate

A mixture of the tert-butyl 3-(pyridin-3-yl)azetidine-1-carboxylate (18.78 g, 80 mmol), sulfuric acid (7.85 g, 80 mmol), PtO₂ (Acros, 1.816 g, 10% by wt) and ethanol (200 mL) was stirred under atmosphere of hydrogen (60 psi) at room temperature for 2 h. After conversion of the starting material, nitrogen was purged through the reaction for 15 min. before it was filtered through a pad of celite, rinsing with ethanol. The volatiles were then removed under reduced pressure to give tert-butyl 3-(piperidin-3-yl)azetidine-1-carboxylate which was used without any further purification.

### Step 3: (R)-(7-(1-benzyl-1H-pyrrolo[2,3-b]pyridin-4-yl)-5-chloro-2,3-dihydrobenzofuran-2-yl)(piperazin-1-yl)methanone and (S)-(7-(1-benzyl-1H-pyrrolo[2,3-b]pyridin-4-yl)-5-chloro-2,3-dihydrobenzofuran-2-yl)(piperazin-1-yl)methanone

The title compound was synthesized by dissolving the product of General Procedures A and B, tert-butyl 4-(5-chloro-7-(1H-pyrrolo[2,3-b]pyridin-4-yl)-2,3-dihydrobenzofuran-2-carbonyl)piperazine-1-carboxylate (20 mg, 0.056 mmol), in THF (0.5 mL) and cooling to 0° C. before the addition of sodium hydride (60% dispersion in oil, 2.7 mg, 0.11 mmol). The mixture was stirred for 15 min before the addition of benzyl bromide (14.3 mg, 0.084 mmol) and subsequent warming to rt The reaction mixture was quenched with NH₄Cl solution and extracted with EtOAc (3×10 mL) and purified by column chromatography eluting with 0 to 70% EtOAc in hexanes. The sequence was then finished with General Procedure D to give the desired compound as the HCl salt. ¹H NMR (400 MHz, CD₃OD, hydrochloric acid salt): δ 8.49 (d, J=6.1 Hz, 1H), 7.86 (d, J=6.2 Hz, 1H), 7.75 (d, J=3.7 Hz, 1H), 7.57 (d, J=2.2 Hz, 1H), 7.51-7.46 (m, 1H), 7.44-7.31 (m, 3H), 7.29-7.22 (m, 2H), 7.14 (d, J=3.7 Hz, 1H), 5.88 (t, J=8.3 Hz, 1H), 5.69 (s, 2H), 3.99-3.64 (m, 6H), 3.29-3.08 (m, 4H); [M+H] 473.0.

### Example 105

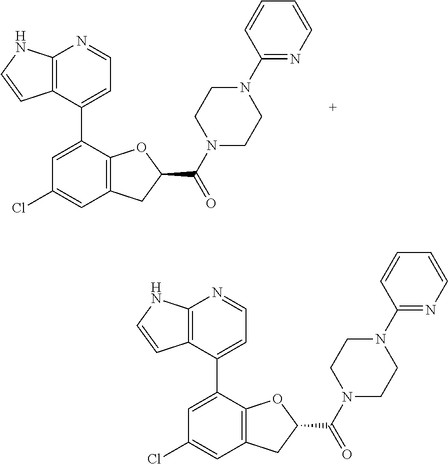 [SMILES: O=C([C@H]1Cc2cc(Cl)cc(-c3ccnc4[nH]ccc34)c2O1)N1CCN(c2ccccn2)CC1 ; O=C([C@@H]1Cc2cc(Cl)cc(-c3ccnc4[nH]ccc34)c2O1)N1CCN(c2ccccn2)CC1]

### (R)-(5-Chloro-7-(1H-pyrrolo[2,3-b]pyridin-4-yl)-2,3-dihydrobenzofuran-2-yl)(4-(pyridin-2-yl)piperazin-1-yl)methanone and (S)-(5-Chloro-7-(1H-pyrrolo[2,3-b]pyridin-4-yl)-2,3-dihydrobenzofuran-2-yl)(4-(pyridin-2-yl)piperazin-1-yl)methanone

The title compound was synthesized using precursor I (step 5), general procedure A and C using 1-(2-pyridyl)piperazine. The final product was purified by column chromatography eluting with 20 to 100% EtOAc in hexanes to give the desired compound as the freebase. ¹H NMR (400 MHz, CDCl₃): δ 10.69 (brs, 1H), 8.33 (d, J=5.0 Hz, 1H), 8.22-8.11 (m, 1H), 7.48-7.38 (m, 2H), 7.33 (d, J=3.5 Hz, 1H), 7.29-7.22 (m, 2H), 6.64 (dd, J=7.0, 4.9 Hz, 1H), 6.58 (d, J=3.5 Hz, 1H), 6.52 (d, J=8.6 Hz, 1H), 5.50 (dd, J=9.9, 7.0 Hz, 1H), 4.06-3.90 (m, 2H), 3.83-3.63 (m, 2H), 3.63-3.28 (m, 5H), 3.21-3.07 (m, 1H); [M+H] 460.0.

### Example 106

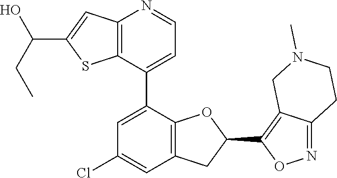 [SMILES: CCC(O)c1cc2nccc(-c3cc(Cl)cc4c3O[C@@H](c3onc5c3CN(C)CC5)C4)c2s1]

### 1-(7-((R)-5-chloro-2-(5-methyl-4,5,6,7-tetrahydroisoxazolo[4,3-c]pyridin-3-yl)-2,3-dihydrobenzofuran-7-yl)thieno[3,2-b]pyridin-2-yl)propan-1-ol

The title compound was synthesized using precursor III, general procedure A, C and E using 1-(7-(4,4,5,5-tetramethyl-1,3,2-dioxaborolan-2-yl)thieno[3,2-b]pyridin-2-yl)propan-1-ol. Reductive amination using paraformaldehyde and sodium triacetoxyborohydride gave the desired compound. [M+H] 482.1.

### Example 107

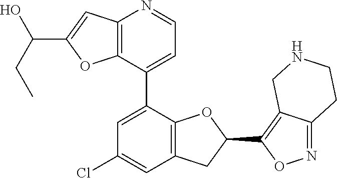 [SMILES: CCC(O)c1cc2nccc(-c3cc(Cl)cc4c3O[C@@H](c3onc5c3CNCC5)C4)c2o1]

### 1-(7-((R)-5-chloro-2-(4,5,6,7-tetrahydroisoxazolo[4,3-c]pyridin-3-yl)-2,3-dihydrobenzofuran-7-yl)furo[3,2-b]pyridin-2-yl)propan-1-ol

The title compound was synthesized using precursor III, general procedure A, C and D using 1-(7-(4,4,5,5-tetramethyl-1,3,2-dioxaborolan-2-yl)furo[3,2-b]pyridin-2-yl)propan-1-ol to give the desired compound as the HCl salt. [M+H] 452.1.

### Example 108

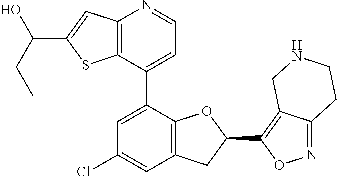 [SMILES: CCC(O)c1cc2nccc(-c3cc(Cl)cc4c3O[C@@H](c3onc5c3CNCC5)C4)c2s1]

### 1-(7-((R)-5-chloro-2-(4,5,6,7-tetrahydroisoxazolo[4,3-c]pyridin-3-yl)-2,3-dihydrobenzofuran-7-yl)thieno[3,2-b]pyridin-2-yl)propan-1-ol

The title compound was synthesized using precursor III, general procedure A, C and D using 1-(7-(4,4,5,5-tetramethyl-1,3,2-dioxaborolan-2-yl)thieno[3,2-b]pyridin-2-yl)propan-1-ol to give the desired compound as the HCl salt. [M+H] 468.0.

### Example 109

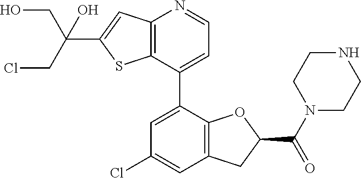 [SMILES: O=C([C@H]1Cc2cc(Cl)cc(-c3ccnc4cc(C(O)(CO)CCl)sc34)c2O1)N1CCNCC1]

### ((2R)-5-chloro-7-(2-(1-chloro-2,3-dihydroxypropan-2-yl)thieno[3,2-b]pyridin-7-yl)-2,3-dihydrobenzofuran-2-yl)(piperazin-1-yl)methanone

The title compound was synthesized using precursor II, general procedure A, C and D using 3-chloro-2-(7-(4,4,5,5-tetramethyl-1,3,2-dioxaborolan-2-yl)thieno[3,2-b]pyridin-2-yl)propane-1,2-diol to give the desired compound as the HCl salt. [M+H] 507.9.

### Example 110

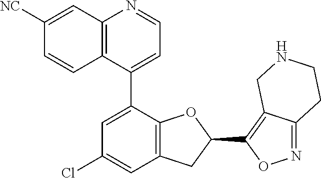 [SMILES: N#Cc1ccc2c(-c3cc(Cl)cc4c3O[C@@H](c3onc5c3CNCC5)C4)ccnc2c1]

### (R)-4-(5-chloro-2-(4,5,6,7-tetrahydroisoxazolo[4,3-c]pyridin-3-yl)-2,3-dihydrobenzofuran-7-yl)quinoline-7-carbonitrile

The title compound was synthesized using precursor III, general procedure A, C and D using 4-(4,4,5,5-tetramethyl-1,3,2-dioxaborolan-2-yl)quinoline-7-carbonitrile to give the desired compound as the HCl salt. [M+H] 429.1.

### Example 111

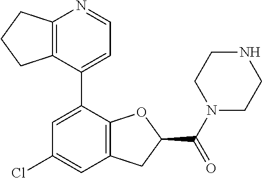 [SMILES: O=C([C@H]1Cc2cc(Cl)cc(-c3ccnc4c3CCC4)c2O1)N1CCNCC1]

### (R)-(5-chloro-7-(6,7-dihydro-5H-cyclopenta[b]pyridin-4-yl)-2,3-dihydrobenzofuran-2-yl)(piperazin-1-yl)methanone

The title compound was synthesized using precursor II, general procedure A, C and D using 4-(4,4,5,5-tetramethyl-1,3,2-dioxaborolan-2-yl)-6,7-dihydro-5H-cyclopenta[b]pyridine to give the desired compound as the HCl salt. [M+H] 384.1.

### Example 112

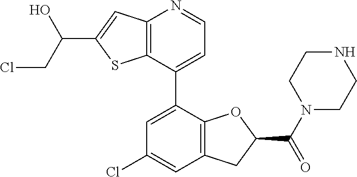 [SMILES: O=C([C@H]1Cc2cc(Cl)cc(-c3ccnc4cc(C(O)CCl)sc34)c2O1)N1CCNCC1]

### ((2R)-5-chloro-7-(2-(2-chloro-1-hydroxyethyl)thieno[3,2-b]pyridin-7-yl)-2,3-dihydrobenzofuran-2-yl)(piperazin-1-yl)methanone

The title compound was synthesized using precursor II, general procedure A, C and D using 2-chloro-1-(7-(4,4,5,5-tetramethyl-1,3,2-dioxaborolan-2-yl)thieno[3,2-b]pyridin-2-yl)ethan-1-ol to give the desired compound as the HCl salt. [M+H] 477.9.

### Example 113

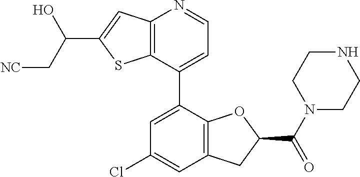 [SMILES: N#CCC(O)c1cc2nccc(-c3cc(Cl)cc4c3O[C@@H](C(=O)N3CCNCC3)C4)c2s1]

### 3-(7-((R)-5-chloro-2-(piperazine-1-carbonyl)-2,3-dihydrobenzofuran-7-yl)thieno[3,2-b]pyridin-2-yl)-3-hydroxypropanenitrile

The title compound was synthesized using precursor II, general procedure A, C and D using 2-hydroxy-2-(7-(4,4,5,5-tetramethyl-1,3,2-dioxaborolan-2-yl)thieno[3,2-b]pyridin-2-yl)acetonitrile to give the desired compound as the HCl salt. [M+H] 469.0.

### Example 114

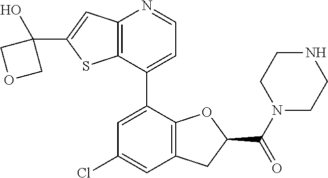 [SMILES: O=C([C@H]1Cc2cc(Cl)cc(-c3ccnc4cc(C5(O)COC5)sc34)c2O1)N1CCNCC1]

### (R)-(5-chloro-7-(2-(3-hydroxyoxetan-3-yl)thieno[3,2-b]pyridin-7-yl)-2,3-dihydrobenzofuran-2-yl)(piperazin-1-yl)methanone

The title compound was synthesized using precursor II, general procedure A, C and D using 3-(7-(4,4,5,5-tetramethyl-1,3,2-dioxaborolan-2-yl)thieno[3,2-b]pyridin-2-yl)oxetan-3-ol to give the desired compound as the HCl salt. [M+H] 472.0.

### Example 115

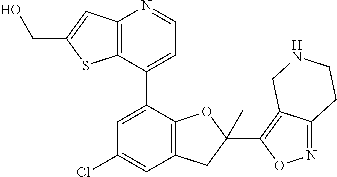 [SMILES: CC1(c2onc3c2CNCC3)Cc2cc(Cl)cc(-c3ccnc4cc(CO)sc34)c2O1]

### (7-(5-chloro-2-methyl-2-(4,5,6,7-tetrahydroisoxazolo[4,3-c]pyridin-3-yl)-2,3-dihydrobenzofuran-7-yl)thieno[3,2-b]pyridin-2-yl)methanol

The title compound was synthesized using precursor I, general procedure A, C and D using (7-(4,4,5,5-tetramethyl-1,3,2-dioxaborolan-2-yl)thieno[3,2-b]pyridin-2-yl)methanol to give the desired compound as the HCl salt. [M+H] 454.1.

### Example 116

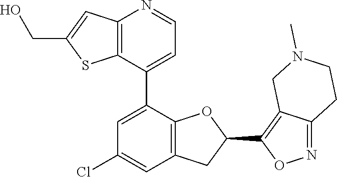 [SMILES: CN1CCc2noc([C@H]3Cc4cc(Cl)cc(-c5ccnc6cc(CO)sc56)c4O3)c2C1]

### (R)-(7-(5-chloro-2-(5-methyl-4,5,6,7-tetrahydroisoxazolo[4,3-c]pyridin-3-yl)-2,3-dihydrobenzofuran-7-yl)thieno[3,2-b]pyridin-2-yl)methanol

The title compound was synthesized using precursor III, general procedure A, C and E using (7-(4,4,5,5-tetramethyl-1,3,2-dioxaborolan-2-yl)thieno[3,2-b]pyridin-2-yl)methanol. Reductive amination using paraformaldehyde and sodium triacetoxyborohydride gave the desired product. [M+H] 454.1.

### Example 117

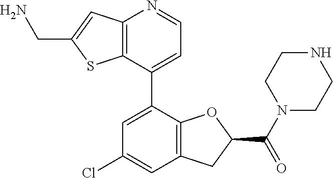 [SMILES: NCc1cc2nccc(-c3cc(Cl)cc4c3O[C@@H](C(=O)N3CCNCC3)C4)c2s1]

### (R)-(7-(2-(aminomethyl)thieno[3,2-b]pyridin-7-yl)-5-chloro-2,3-dihydrobenzofuran-2-yl)(piperazin-1-yl)methanone

The title compound was synthesized using precursor II, general procedure A, C and D using tert-butyl ((7-(4,4,5,5-tetramethyl-1,3,2-dioxaborolan-2-yl)thieno[3,2-b]pyridin-2-yl)methyl)carbamate to give the desired compound as the HCl salt. [M+H] 429.0.

### Example 118

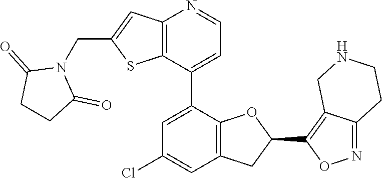 [SMILES: O=C1CCC(=O)N1Cc1cc2nccc(-c3cc(Cl)cc4c3O[C@@H](c3onc5c3CNCC5)C4)c2s1]

### (R)-1-((7-(5-chloro-2-(4,5,6,7-tetrahydroisoxazolo[4,3-c]pyridin-3-yl)-2,3-dihydrobenzofuran-7-yl)thieno[3,2-b]pyridin-2-yl)methyl)pyrrolidine-2,5-dione

The title compound was synthesized using precursor III, general procedure G and D using 1-((7-chlorothieno[3,2-b]pyridin-2-yl)methyl)pyrrolidine-2,5-dione to give the desired compound as the HCl salt. 1H NMR (400 MHz, Methanol-d4) δ 8.89 (s, 1H), 7.93 (s, 1H), 7.73 (s, 1H), 7.61 (s, 2H), 6.22 (s, 1H), 5.04 (s, 2H), 4.28-4.12 (m, 1H), 3.89-3.43 (m, 5H), 3.18-3.08 (m, 2H), 2.77 (s, 4H). [M+H] 521.0.

### Example 119

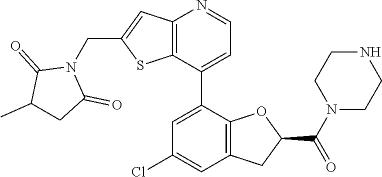 [SMILES: CC1CC(=O)N(Cc2cc3nccc(-c4cc(Cl)cc5c4O[C@@H](C(=O)N4CCNCC4)C5)c3s2)C1=O]

### 1-((7-((R)-5-chloro-2-(piperazine-1-carbonyl)-2,3-dihydrobenzofuran-7-yl)thieno[3,2-b]pyridin-2-yl)methyl)-3-methylpyrrolidine-2,5-dione

The title compound was synthesized using precursor II, general procedure B and D using 1-((7-chlorothieno[3,2-b]pyridin-2-yl)methyl)-3-methylpyrrolidine-2,5-dione to give the desired compound as the HCl salt. 1H NMR (400 MHz, Methanol-d4) δ 8.91 (d, J=6.2 Hz, 1H), 8.10 (d, J=6.0 Hz, 1H), 7.75 (s, 1H), 7.63 (d, J=5.0 Hz, 1H), 7.54 (d, J=2.1 Hz, 1H), 5.89 (dd, J=9.5, 7.2 Hz, 1H), 5.10 (s, 2H), 3.88-3.51 (m, 9H), 3.28-2.96 (m, 4H), 1.31 (d, J=6.9 Hz, 3H). [M+H] 525.0.

### Example 120

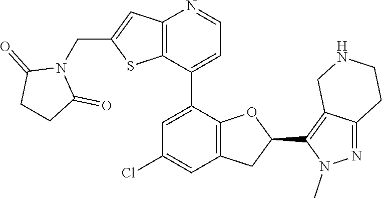 [SMILES: Cn1nc2c(c1[C@H]1Cc3cc(Cl)cc(-c4ccnc5cc(CN6C(=O)CCC6=O)sc45)c3O1)CNCC2]

### (R)-1-((7-(5-chloro-2-(2-methyl-4,5,6,7-tetrahydro-2H-pyrazolo[4,3-c]pyridin-3-yl)-2,3-dihydrobenzofuran-7-yl)thieno[3,2-b]pyridin-2-yl)methyl)pyrrolidine-2,5-dione

The title compound was synthesized using precursor IV, general procedure C and D using 1-((7-(4,4,5,5-tetramethyl-1,3,2-dioxaborolan-2-yl)thieno[3,2-b]pyridin-2-yl)methyl)pyrrolidine-2,5-dione to give the desired compound as the HCl salt.

### Example 121

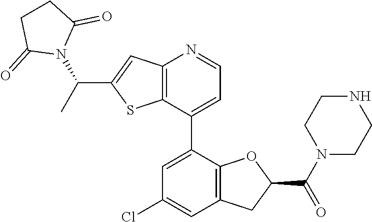 [SMILES: C[C@@H](c1cc2nccc(-c3cc(Cl)cc4c3O[C@@H](C(=O)N3CCNCC3)C4)c2s1)N1C(=O)CCC1=O]

### 1-((S)-1-(7-((R)-5-chloro-2-(piperazine-1-carbonyl)-2,3-dihydrobenzofuran-7-yl)thieno[3,2-b]pyridin-2-yl)ethyl)pyrrolidine-2,5-dione

The title compound was synthesized using precursor II, general procedure B and D using (S)-1-(1-(7-chlorothieno[3,2-b]pyridin-2-yl)ethyl)pyrrolidine-2,5-dione to give the desired compound as the HCl salt. 1H NMR (400 MHz, Methanol-d4) δ 8.95-8.88 (m, 1H), 8.10 (d, J=6.0 Hz, 1H), 7.75 (s, 1H), 7.63 (s, 1H), 7.54 (s, 1H), 5.93-5.79 (m, 1H), 4.90-4.81 (m, 1H), 3.92-3.42 (m, 8H), 3.32-3.25 (m, 2H), 2.86-2.68 (m, 4H), 1.94 (d, J=7.2 Hz, 3H). [M+H] 525.0.

### Example 122

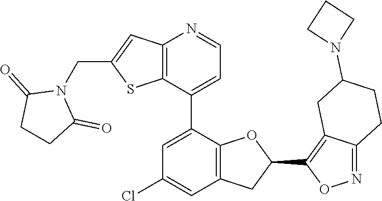 [SMILES: O=C1CCC(=O)N1Cc1cc2nccc(-c3cc(Cl)cc4c3O[C@@H](c3onc5c3CC(N3CCC3)CC5)C4)c2s1]

### 1-((7-((2R)-2-(5-(azetidin-1-yl)-4,5,6,7-tetrahydrobenzo[c]isoxazol-3-yl)-5-chloro-2,3-dihydrobenzofuran-7-yl)thieno[3,2-b]pyridin-2-yl)methyl)pyrrolidine-2,5-dione

The title compound was synthesized using precursor V, general procedure C and D using 1-((7-(4,4,5,5-tetramethyl-1,3,2-dioxaborolan-2-yl)thieno[3,2-b]pyridin-2-yl)methyl)pyrrolidine-2,5-dione to give the desired compound as the HCl salt. 1H NMR (400 MHz, CD₃OD): δ ppm 8.60 (dd, J=5.0, 1.7 Hz, 1H), 7.49-7.36 (m, 4H), 6.04 (ddd, J=10.1, 6.1, 4.2 Hz, 1H), 4.92 (s, 2H), 3.80-3.69 (m, 1H), 3.56-3.44 (m, 1H), 3.11 (q, J=7.1 Hz, 1H), 3.07-2.92 (m, 3H), 2.82-2.67 (m, 5H), 2.65-2.26 (m, 3H), 2.04-1.88 (m, 3H), 1.85-1.71 (m, 1H), 1.53-1.36 (m, 1H). [M+H] 575.0.

### Example 123

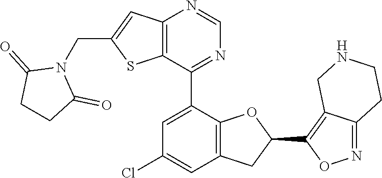 [SMILES: O=C1CCC(=O)N1Cc1cc2ncnc(-c3cc(Cl)cc4c3O[C@@H](c3onc5c3CNCC5)C4)c2s1]

### (R)-1-((4-(5-chloro-2-(4,5,6,7-tetrahydroisoxazolo[4,3-c]pyridin-3-yl)-2,3-dihydrobenzofuran-7-yl)thieno[3,2-d]pyrimidin-6-yl)methyl)pyrrolidine-2,5-dione

The title compound was synthesized using precursor III, general procedure G and D using 1-((4-chlorothieno[3,2-d]pyrimidin-6-yl)methyl)pyrrolidine-2,5-dione to give the desired compound as the HCl salt. 1H NMR (400 MHz, CD₃OD): δ ppm 9.20 (s, 1H), 7.66 (d, J=2.2 Hz, 1H), 7.55 (d, J=5.8 Hz, 2H), 6.22 (dd, J=9.8, 6.7 Hz, 1H), 5.00 (d, J=3.5 Hz, 2H), 4.31 (s, 1H), 3.86 (dd, J=16.3, 9.7 Hz, 1H), 3.77-3.70 (m, 1H), 3.68-3.61 (m, 2H), 3.61-3.51 (m, 3H), 3.14 (t, J=6.4 Hz, 2H), 2.81-2.72 (m, 2H).

### Example 124

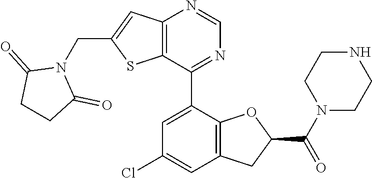 [SMILES: O=C([C@H]1Cc2cc(Cl)cc(-c3ncnc4cc(CN5C(=O)CCC5=O)sc34)c2O1)N1CCNCC1]

### (R)-1-((4-(5-chloro-2-(piperazine-1-carbonyl)-2,3-dihydrobenzofuran-7-yl)thieno[3,2-d]pyrimidin-6-yl)methyl)pyrrolidine-2,5-dione

The title compound was synthesized using precursor II, general procedure B and D using 1-((4-chlorothieno[3,2-d]pyrimidin-6-yl)methyl)pyrrolidine-2,5-dione to give the desired compound as the HCl salt. 1H NMR (400 MHz, CD₃OD): δ ppm 9.22 (s, 1H), 7.62 (d, J=2.3 Hz, 1H), 7.59 (s, 1H), 7.53 (s, 1H), 5.90 (dd, J=10.0, 7.3 Hz, 1H), 5.07 (s, 2H), 3.77-3.71 (m, 3H), 3.71-3.63 (m, 3H), 3.61-3.56 (m, 2H), 3.24 (s, 2H), 2.83-2.76 (m, 4H). [M+H] 513.0.

### Example 125

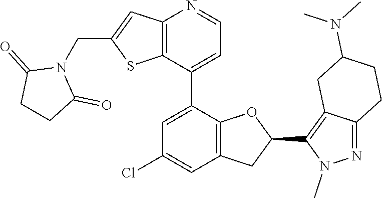 [SMILES: CN(C)C1CCc2nn(C)c([C@H]3Cc4cc(Cl)cc(-c5ccnc6cc(CN7C(=O)CCC7=O)sc56)c4O3)c2C1]

### 1-((7-((2R)-5-chloro-2-(5-(dimethylamino)-2-methyl-4,5,6,7-tetrahydro-2H-indazol-3-yl)-2,3-dihydrobenzofuran-7-yl)thieno[3,2-b]pyridin-2-yl)methyl)pyrrolidine-2,5-dione

The title compound was synthesized using precursor VI, general procedure G and 1-((7-chlorothieno[3,2-b]pyridin-2-yl)methyl)pyrrolidine-2,5-dione to give the desired compound. 1H NMR (400 MHz, CD₃OD) δ 8.47 (dd, J=5.0, 3.7 Hz, 1H), 7.40-7.20 (m, 3H), 6.65-6.44 (m, 1H), 5.77 (ddd, J=9.6, 7.4, 5.4 Hz, 1H), 4.80 (d, J=3.3 Hz, 2H), 2.60 (d, J=2.0 Hz, 4H), 2.55-2.31 (m, 4H), 2.18 (d, J=4.4 Hz, 3H), 2.12-1.85 (m, 9H), 1.53 (dq, J=17.2, 6.9, 5.5 Hz, 2H). [M+H] 576.1.

### Example 126

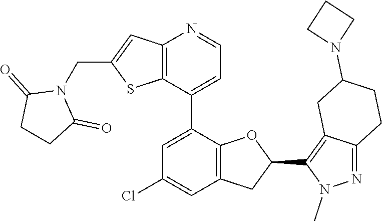 [SMILES: Cn1nc2c(c1[C@H]1Cc3cc(Cl)cc(-c4ccnc5cc(CN6C(=O)CCC6=O)sc45)c3O1)CC(N1CCC1)CC2]

### 1-((7-((2R)-2-(5-(azetidin-1-yl)-2-methyl-4,5,6,7-tetrahydro-2H-indazol-3-yl)-5-chloro-2,3-dihydrobenzofuran-7-yl)thieno[3,2-b]pyridin-2-yl)methyl)pyrrolidine-2,5-dione

The title compound was synthesized using precursor VI, general procedure G and 1-((7-chlorothieno[3,2-b]pyridin-2-yl)methyl)pyrrolidine-2,5-dione to give the desired compound. 1H NMR (400 MHz, CD₃OD) δ 8.48 (s, 1H), 7.31 (dddd, J=16.7, 8.9, 4.8, 2.7 Hz, 12H), 5.85-5.70 (m, 1H), 4.81 (d, J=5.8 Hz, 2H), 4.56 (d, J=5.6 Hz, 2H), 3.53 (d, J=3.3 Hz, 2H), 3.15-3.07 (m, 1H), 3.00 (dd, J=13.0, 6.8 Hz, 2H), 2.62 (dd, J=6.1, 1.0 Hz, 2H), 2.56-2.28 (m, 2H), 2.03 (d, J=1.0 Hz, 9H), 1.96-1.75 (m, 2H). [M+H] 588.1.

### Example 127

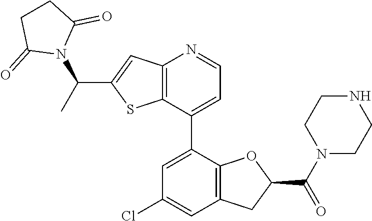 [SMILES: C[C@H](c1cc2nccc(-c3cc(Cl)cc4c3O[C@@H](C(=O)N3CCNCC3)C4)c2s1)N1C(=O)CCC1=O]

### 1-((R)-1-(7-((R)-5-chloro-2-(piperazine-1-carbonyl)-2,3-dihydrobenzofuran-7-yl)thieno[3,2-b]pyridin-2-yl)ethyl)pyrrolidine-2,5-dione

The title compound was synthesized using precursor II, general procedure B and D using (S)-1-(1-(7-chlorothieno[3,2-b]pyridin-2-yl)ethyl)pyrrolidine-2,5-dione to give the desired compound as the HCl salt. 1H NMR (400 MHz, Methanol-d4) δ 8.88 (d, J=6.1 Hz, 1H), 8.04 (d, J=5.9 Hz, 1H), 7.72 (s, 1H), 7.59 (s, 1H), 7.53 (s, 1H), 5.92-5.73 (m, 1H), 4.84-4.80 (m, 1H), 3.84-3.63 (m, 8H), 3.26-3.21 (m, 2H), 2.77-2.73 (m, 4H), 1.94 (d, J=7.1 Hz, 3H). [M+H] 525.0.

### Example 128

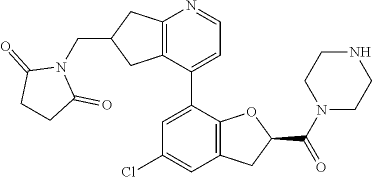 [SMILES: O=C([C@H]1Cc2cc(Cl)cc(-c3ccnc4c3CC(CN3C(=O)CCC3=O)C4)c2O1)N1CCNCC1]

### 1-((4-((R)-5-chloro-2-(piperazine-1-carbonyl)-2,3-dihydrobenzofuran-7-yl)-6,7-dihydro-5H-cyclopenta[b]pyridin-6-yl)methyl)pyrrolidine-2,5-dione

The title compound was synthesized using precursor II, general procedure B, E and F using 1-((4-chloro-6,7-dihydro-5H-cyclopenta[b]pyridin-6-yl)methyl)pyrrolidine-2,5-dione to give the desired compound. 1H NMR (400 MHz, Methanol-d4) δ 8.46-8.40 (m, 1H), 7.71-7.58 (m, 1H), 7.43-7.36 (m, 1H), 7.33-7.25 (m, 1H), 5.85-5.74 (m, 1H), 4.01-3.72 (m, 4H), 3.70-3.53 (m, 5H), 3.53-2.85 (m, 8H), 2.77-2.61 (m, 4H). [M+H] 495.0.

### Example 129

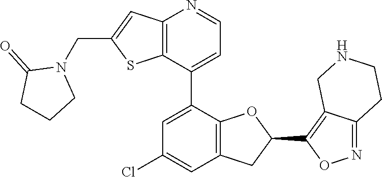 [SMILES: O=C1CCCN1Cc1cc2nccc(-c3cc(Cl)cc4c3O[C@@H](c3onc5c3CNCC5)C4)c2s1]

### (R)-1-((7-(5-chloro-2-(4,5,6,7-tetrahydroisoxazolo[4,3-c]pyridin-3-yl)-2,3-dihydrobenzofuran-7-yl)thieno[3,2-b]pyridin-2-yl)methyl)pyrrolidin-2-one

The title compound was synthesized using precursor III, general procedure G, E and F using 1-((7-chlorothieno[3,2-b]pyridin-2-yl)methyl)pyrrolidin-2-one to give the desired compound. 1H NMR (400 MHz, CD₃OD): δ ppm 8.63 (d, J=4.8 Hz, 1H), 7.47-7.43 (m, 3H), 6.09 (dd, J=10.0, 6.4 Hz, 1H), 4.78 (s, 2H), 3.84-3.44 (m, 6H), 3.17 (bs, 2H), 2.88 (t, J=6.0 Hz, 2H), 2.43 (t, J=8.0 Hz, 2H), 2.08 (d, J=8.0 Hz, 2H). [M+H] 507.0.

### Example 130

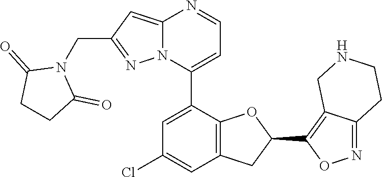 [SMILES: O=C1CCC(=O)N1Cc1cc2nccc(-c3cc(Cl)cc4c3O[C@@H](c3onc5c3CNCC5)C4)n2n1]

### (R)-1-((7-(5-chloro-2-(4,5,6,7-tetrahydroisoxazolo[4,3-c]pyridin-3-yl)-2,3-dihydrobenzofuran-7-yl)pyrazolo[1,5-a]pyrimidin-2-yl)methyl)pyrrolidine-2,5-dione

The title compound was synthesized using precursor III, general procedure G and E using 1-((7-chloropyrazolo[1,5-a]pyrimidin-2-yl)methyl)pyrrolidine-2,5-dione to give the desired compound as the TFA salt. [M+H] 505.0.

### Example 131

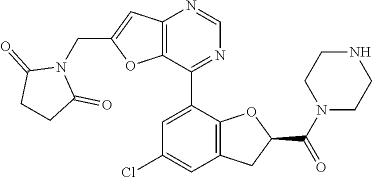 [SMILES: O=C([C@H]1Cc2cc(Cl)cc(-c3ncnc4cc(CN5C(=O)CCC5=O)oc34)c2O1)N1CCNCC1]

### (R)-1-((4-(5-chloro-2-(piperazine-1-carbonyl)-2,3-dihydrobenzofuran-7-yl)furo[3,2-d]pyrimidin-6-yl)methyl)pyrrolidine-2,5-dione

The title compound was synthesized using precursor II, general procedure B and D using 1-((4-chlorofuro[3,2-d]pyrimidin-6-yl)methyl)pyrrolidine-2,5-dione to give the desired compound as the HCl salt. 1H NMR (400 MHz, CD₃OD) δ 7.73 (s, 1H), 7.53 (dd, J=12.3, 7.4 Hz, 1H), 7.44 (s, 1H), 7.23 (d, J=8.6 Hz, 1H), 5.83 (t, J=8.6 Hz, 1H), 4.88 (s, 2H), 3.88 (d, J=16.0 Hz, 2H), 3.60 (ddd, J=6.0, 5.1, 0.9 Hz, 2H), 3.52 (s, 4H), 3.47-3.39 (m, 2H), 3.35 (q, J=7.0 Hz, 1H), 3.15-3.01 (m, 3H). [M+H] 496.1.

### Example 132

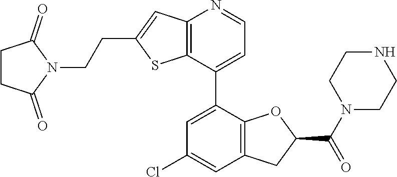 [SMILES: O=C([C@H]1Cc2cc(Cl)cc(-c3ccnc4cc(CCN5C(=O)CCC5=O)sc34)c2O1)N1CCNCC1]

### (R)-1-(2-(7-(5-chloro-2-(piperazine-1-carbonyl)-2,3-dihydrobenzofuran-7-yl)thieno[3,2-b]pyridin-2-yl)ethyl)pyrrolidine-2,5-dione

The title compound was synthesized using precursor II, general procedure B and D using 1-(2-(7-chlorothieno[3,2-b]pyridin-2-yl)ethyl)pyrrolidine-2,5-dione to give the desired compound as the HCl salt. [M+H] 525.0.

### Example 133

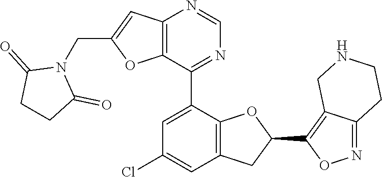 [SMILES: O=C1CCC(=O)N1Cc1cc2ncnc(-c3cc(Cl)cc4c3O[C@@H](c3onc5c3CNCC5)C4)c2o1]

### (R)-1-((4-(5-chloro-2-(4,5,6,7-tetrahydroisoxazolo[4,3-c]pyridin-3-yl)-2,3-dihydrobenzofuran-7-yl)furo[3,2-d]pyrimidin-6-yl)methyl)pyrrolidine-2,5-dione

The title compound was synthesized using precursor III, general procedure B and D using 1-((4-chlorofuro[3,2-d]pyrimidin-6-yl)methyl)pyrrolidine-2,5-dione to give the desired compound as the HCl salt. 1H NMR (400 MHz, CD₃OD) δ 8.94 (s, 1H), 7.73 (d, J=2.2 Hz, 1H), 7.46-7.38 (m, 1H), 6.93 (s, 1H), 6.13 (dd, J=10.1, 6.3 Hz, 1H), 4.81 (d, J=1.0 Hz, 2H), 4.29-4.11 (m, 2H), 3.75 (dd, J=16.4, 10.0 Hz, 1H), 3.61 (ddd, J=6.0, 5.1, 0.9 Hz, 1H), 3.57-3.50 (m, 2H), 3.51-3.42 (m, 1H), 3.41-3.30 (m, 1H), 2.98 (t, J=6.3 Hz, 2H). [M+H] 506.1.

### Example 134

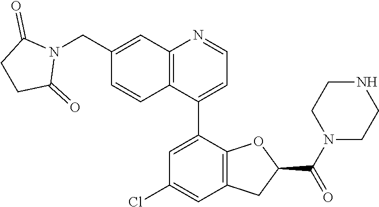 [SMILES: O=C([C@H]1Cc2cc(Cl)cc(-c3ccnc4cc(CN5C(=O)CCC5=O)ccc34)c2O1)N1CCNCC1]

### (R)-1-((4-(5-chloro-2-(piperazine-1-carbonyl)-2,3-dihydrobenzofuran-7-yl)quinolin-7-yl)methyl)pyrrolidine-2,5-dione

The title compound was synthesized using precursor II, general procedure B and D using 1-((4-chloroquinolin-7-yl)methyl)pyrrolidine-2,5-dione to give the desired compound as the HCl salt. 1H NMR (400 MHz, CD₃OD): δ ppm 9.20 (s, 1H), 8.19 (m, 2H), 8.06 (s, 1H), 7.87 (s, 1H), 7.53 (s, 1H), 7.43 (s, 1H), 5.80 (s, 1H), 5.11-4.93 (m, 2H), 3.89 (s, 2H), 3.81-3.37 (m, 8H), 3.12 (s, 2H), 2.82 (d, J=18.8 Hz, 2H).

### Example 135

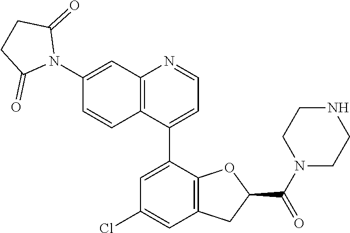 [SMILES: O=C([C@H]1Cc2cc(Cl)cc(-c3ccnc4cc(N5C(=O)CCC5=O)ccc34)c2O1)N1CCNCC1]

### (R)-1-(4-(5-chloro-2-(piperazine-1-carbonyl)-2,3-dihydrobenzofuran-7-yl)quinolin-7-yl)pyrrolidine-2,5-dione

The title compound was synthesized using precursor II, general procedure B and D using 1-(4-chloroquinolin-7-yl)pyrrolidine-2,5-dione to give the desired compound as the HCl salt. 1H NMR (400 MHz, CD₃OD): δ ppm 9.23-9.00 (m, 1H), 8.26-8.02 (m, 2H), 7.88-7.67 (m, 2H), 7.53 (s, 1H), 7.43 (s, 1H), 5.92-5.61 (m, 1H), 3.78 (s, 2H), 4.04-3.50 (m, 6H), 3.30 (s, 2H), 2.85-2.64 (m, 4H). [M+H] 491.0.

### Example 136

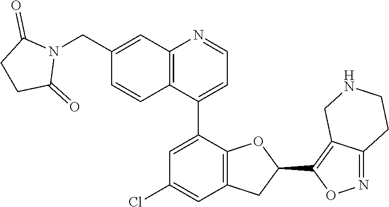 [SMILES: O=C1CCC(=O)N1Cc1ccc2c(-c3cc(Cl)cc4c3O[C@@H](c3onc5c3CNCC5)C4)ccnc2c1]

### (R)-1-((4-(5-chloro-2-(4,5,6,7-tetrahydroisoxazolo[4,3-c]pyridin-3-yl)-2,3-dihydrobenzofuran-7-yl)quinolin-7-yl)methyl)pyrrolidine-2,5-dione

The title compound was synthesized using precursor III, general procedure B and D using 1-((4-chloroquinolin-7-yl)methyl)pyrrolidine-2,5-dione to give the desired compound as the HCl salt. [M+H] 515.0.

### Example 137

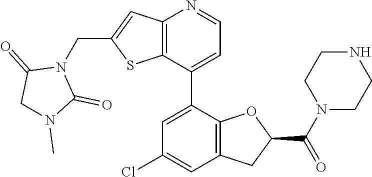 [SMILES: CN1CC(=O)N(Cc2cc3nccc(-c4cc(Cl)cc5c4O[C@@H](C(=O)N4CCNCC4)C5)c3s2)C1=O]

### (R)-3-((7-(5-chloro-2-(piperazine-1-carbonyl)-2,3-dihydrobenzofuran-7-yl)thieno[3,2-b]pyridin-2-yl)methyl)-1-methylimidazolidine-2,4-dione

The title compound was synthesized using precursor II, general procedure B and E using 3-((7-chlorothieno[3,2-b]pyridin-2-yl)methyl)-1-methylimidazolidine-2,4-dione to give the desired compound as the TFA salt. 1H NMR (400 MHz, Methanol-d) δ 8.64 (d, J=4.9 Hz, 1H), 7.53-7.50 (m, 2H), 7.43 (d, J=2.2 Hz, 1H), 7.41-7.39 (m, 1H), 5.74 (dd, J=10.0, 7.0 Hz, 1H), 3.76 (dd, J=16.3, 6.9 Hz, 2H), 4.97 (d, J=0.9 Hz, 2H), 4.04 (s, 2H), 3.55-3.48 (m, 1H), 3.35 (s, 1H), 2.98 (s, 3H), 2.75-2.64 (m, 2H), 2.85-2.75 (m, 1H), 2.54-2.43 (m, 2H). [M+H] 526.0.

### Example 138

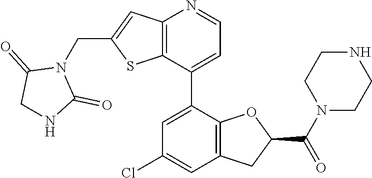 [SMILES: O=C([C@H]1Cc2cc(Cl)cc(-c3ccnc4cc(CN5C(=O)CNC5=O)sc34)c2O1)N1CCNCC1]

### (R)-3-((7-(5-chloro-2-(piperazine-1-carbonyl)-2,3-dihydrobenzofuran-7-yl)thieno[3,2-b]pyridin-2-yl)methyl)imidazolidine-2,4-dione

The title compound was synthesized using precursor II, general procedure B and D using 3-((7-chlorothieno[3,2-b]pyridin-2-yl)methyl)imidazolidine-2,4-dione to give the desired compound as the HCl salt. 1H NMR (400 MHz, CD₃OD): δ ppm 8.95 (s, 1H), 8.13 (s, 1H), 7.81 (s, 1H), 7.61 (s, 1H), 7.54 (s, 1H), 5.92 (s, 1H), 5.11 (s, 2H), 4.08 (s, 2H), 3.89-3.84 (m, 4H), 3.76-3.70 (m, 2H), 3.70-3.60 (m, 4H), 3.35-3.02 (m, 4H). [M+H] 512.1.

### Example 139

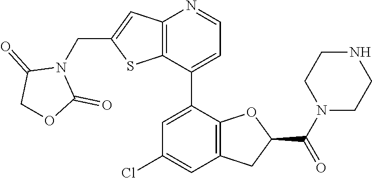 [SMILES: O=C([C@H]1Cc2cc(Cl)cc(-c3ccnc4cc(CN5C(=O)COC5=O)sc34)c2O1)N1CCNCC1]

### (R)-3-((7-(5-chloro-2-(piperazine-1-carbonyl)-2,3-dihydrobenzofuran-7-yl)thieno[3,2-b]pyridin-2-yl)methyl)oxazolidine-2,4-dione

The title compound was synthesized using precursor II, general procedure B and E using 3-((7-chlorothieno[3,2-b]pyridin-2-yl)methyl)oxazolidine-2,4-dione to give the desired compound as the TFA salt. 1H NMR (400 MHz, Methanol-d) δ 8.66 (d, J=4.9 Hz, 1H), 7.57 (s, 1H), 7.54 (d, J=4.9 Hz, 1H), 7.46-7.43 (m, 1H), 7.41 (d, J=2.1 Hz, 1H), 5.75 (dd, J=9.9, 6.9 Hz, 1H), 5.03 (d, J=0.9 Hz, 2H), 3.81-3.70 (m, 2H), 3.57-3.47 (m, 1H), 3.35 (s, 1H), 2.85-2.74 (m, 2H), 2.76-2.63 (m, 2H), 2.57-2.46 (m, 1H). [M+H] 512.9.

### Example 140

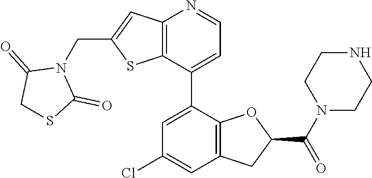 [SMILES: O=C([C@H]1Cc2cc(Cl)cc(-c3ccnc4cc(CN5C(=O)CSC5=O)sc34)c2O1)N1CCNCC1]

### (R)-3-((7-(5-chloro-2-(piperazine-1-carbonyl)-2,3-dihydrobenzofuran-7-yl)thieno[3,2-b]pyridin-2-yl)methyl)thiazolidine-2,4-dione

The title compound was synthesized using precursor II, general procedure B and E using 3-((7-chlorothieno[3,2-b]pyridin-2-yl)methyl)thiazolidine-2,4-dione to give the desired compound as the TFA salt. 1H NMR (400 MHz, Methanol-d) δ 8.65 (d, J=4.9 Hz, 1H), 7.55-7.49 (m, 2H), 7.44-7.41 (m, 1H), 7.41-7.40 (m, 1H), 5.74 (dd, J=9.9, 7.0 Hz, 1H), 5.08 (d, J=0.5 Hz, 1H), 3.80-3.71 (m, 2H), 3.66-3.58 (m, 1H), 3.55-3.40 (m, 2H), 3.35 (s, 4H), 2.79 (ddd, J=12.6, 5.8, 3.3 Hz, 1H), 2.73-2.63 (m, 2H), 2.49 (ddd, J=12.1, 7.9, 3.2 Hz, 1H). [M+H] 529.0.

### Example 141

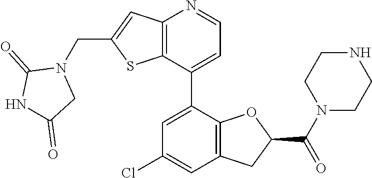 [SMILES: O=C1CN(Cc2cc3nccc(-c4cc(Cl)cc5c4O[C@@H](C(=O)N4CCNCC4)C5)c3s2)C(=O)N1]

### (R)-1-((7-(5-chloro-2-(piperazine-1-carbonyl)-2,3-dihydrobenzofuran-7-yl)thieno[3,2-b]pyridin-2-yl)methyl)imidazolidine-2,4-dione

The title compound was synthesized by treating the title compound from Example 142 with ceric ammonium nitrate and purification by reverse phase HPLC as the TFA salt.

### Example 142

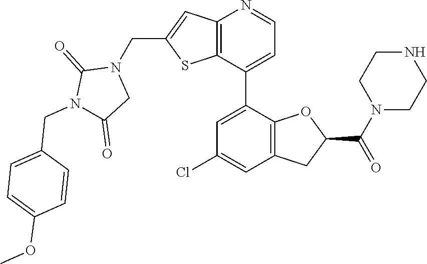 [SMILES: COc1ccc(CN2C(=O)CN(Cc3cc4nccc(-c5cc(Cl)cc6c5O[C@@H](C(=O)N5CCNCC5)C6)c4s3)C2=O)cc1]

### (R)-1-((7-(5-chloro-2-(piperazine-1-carbonyl)-2,3-dihydrobenzofuran-7-yl)thieno[3,2-b]pyridin-2-yl)methyl)-3-(4-methoxybenzyl)imidazolidine-2,4-dione

The title compound was synthesized using precursor II, general procedure B and E using 1-((7-chlorothieno[3,2-b]pyridin-2-yl)methyl)-3-(4-methoxybenzyl)imidazolidine-2,4-dione to give the desired compound as the TFA salt.

### Example 143

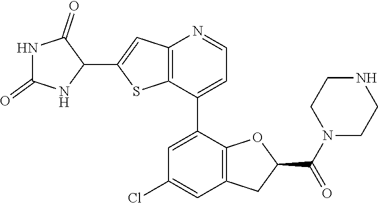 [SMILES: O=C1NC(=O)C(c2cc3nccc(-c4cc(Cl)cc5c4O[C@@H](C(=O)N4CCNCC4)C5)c3s2)N1]

### 5-(7-((R)-5-chloro-2-(piperazine-1-carbonyl)-2,3-dihydrobenzofuran-7-yl)thieno[3,2-b]pyridin-2-yl)imidazolidine-2,4-dione

The title compound was synthesized using precursor II, general procedure B, E and F using 5-(7-chlorothieno[3,2-b]pyridin-2-yl)imidazolidine-2,4-dione to give the desired compound. 1H NMR (400 MHz, CD₃OD, free base): δ ppm 8.74 (d, J=5.2 Hz, 1H), 8.13 (s, 1H), 7.61 (s, 1H), 7.46 (s, 1H), 7.39 (s, 1H), 5.75 (dd, J=10.0, 6.8 Hz, 1H), 3.74-3.39 (m, 7H), 2.77-2.49 (m, 4H). [M-urea] 443.0.

### Example 144

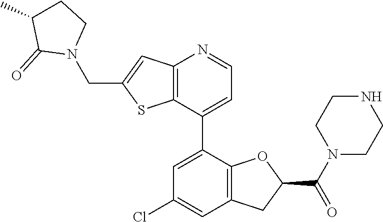 [SMILES: C[C@@H]1CCN(Cc2cc3nccc(-c4cc(Cl)cc5c4O[C@@H](C(=O)N4CCNCC4)C5)c3s2)C1=O]

### (R)-1-((7-((R)-5-chloro-2-(piperazine-1-carbonyl)-2,3-dihydrobenzofuran-7-yl)thieno[3,2-b]pyridin-2-yl)methyl)-3-methylpyrrolidin-2-one

The title compound was synthesized using precursor II, general procedure B and D using 1-((7-chlorothieno[3,2-b]pyridin-2-yl)methyl)-3-methylpyrrolidin-2-one to give the desired compound as the HCl salt. 1H NMR (400 MHz, CD₃OD): δ ppm 8.66-8.59 (m, 1H), 7.53 (d, J=4.8 Hz, 1H), 7.43 (d, J=6.7 Hz, 1H), 7.38 (s, 1H), 7.24 (s, 1H), 5.84-5.73 (m, 1H), 4.97 (s, 2H), 3.76 (s, 1H), 3.49 (dd, J=16.1, 14.4 Hz, 1H), 3.33-3.08 (m, 8H), 2.95-2.80 (m, 2H), 2.57-2.50 (m, 3H), 1.26 (d, J=7.1 Hz, 3H). [M+H] 511.0.

### Example 145

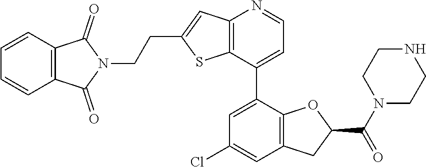 [SMILES: O=C([C@H]1Cc2cc(Cl)cc(-c3ccnc4cc(CCN5C(=O)c6ccccc6C5=O)sc34)c2O1)N1CCNCC1]

### (R)-2-(2-(7-(5-chloro-2-(piperazine-1-carbonyl)-2,3-dihydrobenzofuran-7-yl)thieno[3,2-b]pyridin-2-yl)ethyl)isoindoline-1,3-dione

The title compound was synthesized using precursor II, general procedure B and D using 2-(2-(7-chlorothieno[3,2-b]pyridin-2-yl)ethyl)isoindoline-1,3-dione to give the desired compound as the HCl salt. 1H NMR (400 MHz, Methanol-d4) δ 8.89 (s, 1H), 8.13 (s, 1H), 7.86-7.78 (m, 3H), 7.67-7.51 (m, 4H), 5.91 (t, J=8.3 Hz, 1H), 4.14 (t, J=6.7 Hz, 2H), 3.92-3.62 (m, 6H), 3.30-3.16 (m, 6H). [M+H] 573.0.

### Example 146

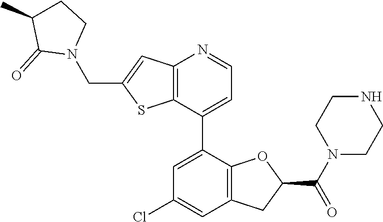 [SMILES: C[C@H]1CCN(Cc2cc3nccc(-c4cc(Cl)cc5c4O[C@@H](C(=O)N4CCNCC4)C5)c3s2)C1=O]

### (S)-1-((7-((R)-5-chloro-2-(piperazine-1-carbonyl)-2,3-dihydrobenzofuran-7-yl)thieno[3,2-b]pyridin-2-yl)methyl)-3-methylpyrrolidin-2-one

The title compound was synthesized using precursor II, general procedure B and D using 1-((7-chlorothieno[3,2-b]pyridin-2-yl)methyl)-3-methylpyrrolidin-2-one to give the desired compound as the HCl salt. 1H NMR (400 MHz, CD₃OD): δ ppm 8.88 (s, 1H), 8.08-8.00 (m, 1H), 7.74-7.52 (m, 2H), 7.34-7.20 (m, 2H), 5.92-5.82 (m, 1H), 3.85 (s, 2H), 3.47 (d, J=7.9 Hz, 2H), 3.42-3.10 (m, 10H), 2.66-2.51 (m, 1H), 2.40-2.25 (m, 1H), 1.83-1.63 (m, 1H), 1.21 (d, J=7.1 Hz, 3H). [M+H] 511.0.

### Example 147

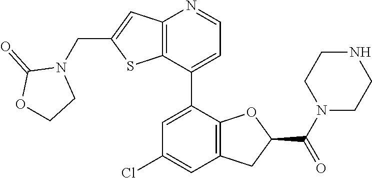 [SMILES: O=C1OCCN1Cc1cc2nccc(-c3cc(Cl)cc4c3O[C@@H](C(=O)N3CCNCC3)C4)c2s1]

### (R)-3-((7-(5-chloro-2-(piperazine-1-carbonyl)-2,3-dihydrobenzofuran-7-yl)thieno[3,2-b]pyridin-2-yl)methyl)oxazolidin-2-one

The title compound was synthesized using precursor II, general procedure B, E and F using 3-((7-chlorothieno[3,2-b]pyridin-2-yl)methyl)oxazolidin-2-one to give the desired compound. [M+H] 499.0.

### Example 148

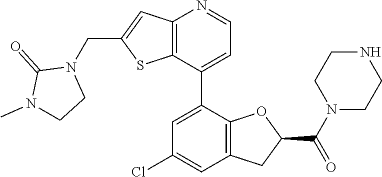 [SMILES: CN1CCN(Cc2cc3nccc(-c4cc(Cl)cc5c4O[C@@H](C(=O)N4CCNCC4)C5)c3s2)C1=O]

### (R)-1-((7-(5-chloro-2-(piperazine-1-carbonyl)-2,3-dihydrobenzofuran-7-yl)thieno[3,2-b]pyridin-2-yl)methyl)-3-methylimidazolidin-2-one

The title compound was synthesized using precursor II, general procedure B and D using 1-((7-chlorothieno[3,2-b]pyridin-2-yl)methyl)-3-methylimidazolidin-2-one to give the desired compound as the HCl salt. 1H NMR (400 MHz, CD₃OD): δ ppm 8.17-7.89 (m, 2H), 7.60-7.54 (m, 1H), 7.49 (s, 2H), 5.94-5.74 (m, 1H), 4.04-3.94 (m, 1H), 3.90-3.71 (m, 3H), 3.72-3.49 (m, 4H), 3.24-3.04 (m, 2H), 2.82 (s, 3H).

### Example 149

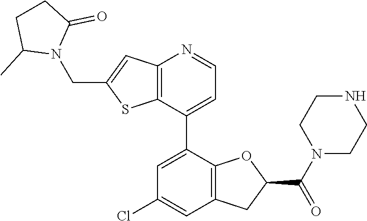 [SMILES: CC1CCC(=O)N1Cc1cc2nccc(-c3cc(Cl)cc4c3O[C@@H](C(=O)N3CCNCC3)C4)c2s1]

### 1-((7-((R)-5-chloro-2-(piperazine-1-carbonyl)-2,3-dihydrobenzofuran-7-yl)thieno[3,2-b]pyridin-2-yl)methyl)-5-methylpyrrolidin-2-one

The title compound was synthesized using precursor II, general procedure B and D using 1-((7-chlorothieno[3,2-b]pyridin-2-yl)methyl)-5-methylpyrrolidin-2-one to give the desired compound as the HCl salt. 1H NMR (400 MHz, Methanol-d4) δ 8.96-8.86 (m, 1H), 8.10 (d, J=6.0 Hz, 1H), 7.74 (s, 1H), 7.63 (s, 1H), 7.54 (d, J=1.5 Hz, 1H), 5.93-5.84 (m, 1H), 5.04 (dd, J=16.5, 5.1 Hz, 1H), 4.85-4.82 (m, 2H), 3.91-3.50 (m, 10H), 3.26-3.12 (m, 2H), 2.57-2.38 (m, 1H), 1.77-1.60 (m, 1H), 1.29 (dd, J=6.3, 1.6 Hz, 3H). [M+H] 511.0.

### Example 150

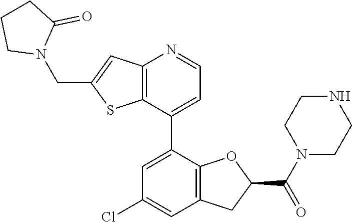 [SMILES: O=C1CCCN1Cc1cc2nccc(-c3cc(Cl)cc4c3O[C@@H](C(=O)N3CCNCC3)C4)c2s1]

### (R)-1-((7-(5-chloro-2-(piperazine-1-carbonyl)-2,3-dihydrobenzofuran-7-yl)thieno[3,2-b]pyridin-2-yl)methyl)pyrrolidin-2-one

The title compound was synthesized using precursor II, general procedure B, E and F using 1-((7-chlorothieno[3,2-b]pyridin-2-yl)methyl)pyrrolidin-2-one to give the desired compound. 1H NMR (400 MHz, CD₃OD): δ ppm 8.63 (bs, 1H), 7.54 (s, 1H), 7.45 (s, 1H), 7.44 (s, 1H), 7.39 (s, 1H), 5.74 (dd, J=9.6, 6.8 Hz, 1H), 4.59 (s, 2H), 3.74-3.39 (m, 10H), 2.43 (t, J=8.0 Hz, 2H), 2.09 (quin, J=7.2 Hz, 2H). [M+H] 497.0.

### Example 151

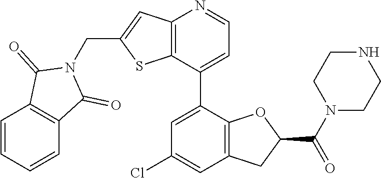 [SMILES: O=C([C@H]1Cc2cc(Cl)cc(-c3ccnc4cc(CN5C(=O)c6ccccc6C5=O)sc34)c2O1)N1CCNCC1]

### (R)-2-((7-(5-chloro-2-(piperazine-1-carbonyl)-2,3-dihydrobenzofuran-7-yl)thieno[3,2-b]pyridin-2-yl)methyl)isoindoline-1,3-dione

The title compound was synthesized using the product of Example 10 after general procedure E. To diisopropyl azodicarboxylate (0.18 g, 0.90 mmol) and phthalimide (0.13 g, 0.86 mmol) in THF (5 mL) was added triphenylphosphine polymer bound (1.14 mmol) and tert-butyl 4-[5-chloro-7-[2-(hydroxymethyl)thieno[3,2-b]pyridin-7-yl]-2,3-dihydrobenzofuran-2-carbonyl]piperazine-1-carboxylate (0.10 g, 0.50 mmol). Stirred at 60° C. overnight. Filtered all solids and washed the filtrate twice with EtOAc. Combined all organic phases and washed with water and brine. Concentrated and purified by silica gel column with hexanes:EtOAc (70% to 100%). The product was subjected to general procedure D to give the desired compound as the HCl salt. 1H NMR (400 MHz, CD₃OD): δ ppm 8.66 (d, J=5.1 Hz, 1H), 7.92 (dd, J=5.6, 2.9 Hz, 2H), 7.84 (dd, J=5.6, 3.0 Hz, 2H), 7.55 (d, J=5.0 Hz, 2H), 7.43 (d, J=7.8 Hz, 2H), 5.75 (dd, J=10.0, 7.2 Hz, 1H), 5.20 (s, 2H), 4.86-4.71 (m, 1H), 3.90-3.56 (m, 3H), 3.49 (d, J=7.7 Hz, 2H), 3.20-3.06 (m, 4H). [M+H] 559.0.

### Example 152

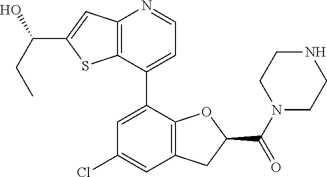 [SMILES: CC[C@H](O)c1cc2nccc(-c3cc(Cl)cc4c3O[C@@H](C(=O)N3CCNCC3)C4)c2s1]

### ((R)-5-chloro-7-(2-((S)-1-hydroxypropyl)thieno[3,2-b]pyridin-7-yl)-2,3-dihydrobenzofuran-2-yl)(piperazin-1-yl)methanone

The title compound was synthesized using precursor II, general procedure B and D using (S)-1-(7-chlorothieno[3,2-b]pyridin-2-yl)propan-1-ol to give the desired compound as an HCl salt. 1H NMR (400 MHz, Methanol-d4) δ 8.90 (s, 1H), 8.14 (d, J=5.4 Hz, 1H), 7.68 (d, J=8.6 Hz, 1H), 7.62 (d, J=7.1 Hz, 1H), 7.55 (d, J=1.8 Hz, 1H), 5.92 (t, J=8.1 Hz, 1H), 5.10 (dd, J=7.5, 4.9 Hz, 1H), 3.93-3.54 (m, 6H), 3.28-3.11 (m, 4H), 2.05-1.84 (m, 2H), 1.05 (t, J=7.3 Hz, 3H). [M+H] 458.0.

### Example 153

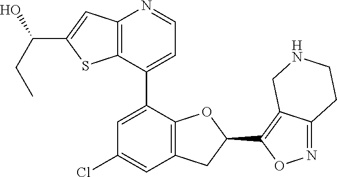 [SMILES: CC[C@H](O)c1cc2nccc(-c3cc(Cl)cc4c3O[C@@H](c3onc5c3CNCC5)C4)c2s1]

### (S)-1-(7-((R)-5-chloro-2-(4,5,6,7-tetrahydroisoxazolo[4,3-c]pyridin-3-yl)-2,3-dihydrobenzofuran-7-yl)thieno[3,2-b]pyridin-2-yl)propan-1-ol

The title compound was synthesized using precursor III, general procedure B and D using (S)-1-(7-chlorothieno[3,2-b]pyridin-2-yl)propan-1-ol to give the desired compound as an HCl salt. 1H NMR (400 MHz, Methanol-d) δ 8.61 (d, J=5.0 Hz, 1H), 7.52-7.49 (m, 1H), 7.49-7.43 (m, 2H), 7.43 (d, J=5.0 Hz, 1H), 7.39 (d, J=1.0 Hz, 1H), 6.09 (dd, J=9.9, 6.2 Hz, 1H), 4.92 (t, J=6.3 Hz, 1H), 3.81 (dd, J=16.4, 9.9 Hz, 1H), 3.63 (s, 1H), 3.61-3.51 (m, 1H), 2.97 (t, J=6.0 Hz, 2H), 2.74 (t, J=6.0 Hz, 2H), 1.88 (p, J=7.2 Hz, 2H), 1.01 (t, J=7.4 Hz, 3H). [M+H] 468.0.

### Example 154

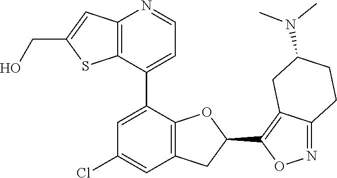 [SMILES: CN(C)[C@@H]1CCc2noc([C@H]3Cc4cc(Cl)cc(-c5ccnc6cc(CO)sc56)c4O3)c2C1]

### (7-((R)-5-chloro-2-((R)-5-(dimethylamino)-4,5,6,7-tetrahydrobenzo[c]isoxazol-3-yl)-2,3-dihydrobenzofuran-7-yl)thieno[3,2-b]pyridin-2-yl)methanol

The title compound was synthesized using precursor V, general procedure C and D using (7-chlorothieno[3,2-b]pyridin-2-yl)methanol to give the desired compound. 1H NMR (400 MHz, CD₃OD): δ ppm 8.60 (dd, J=5.0, 4.0 Hz, 1H), 7.53 (dq, J=1.8, 0.8 Hz, 1H), 7.50-7.43 (m, 2H), 7.41 (t, J=1.2 Hz, 1H), 6.14-6.04 (m, 1H), 4.89 (dd, J=1.1, 0.4 Hz, 2H), 3.79 (ddq, J=16.3, 9.9, 1.1 Hz, 1H), 3.65-3.50 (m, 1H), 2.96-2.81 (m, 1H), 2.69-2.44 (m, 3H), 2.36-2.15 (m, 1H), 2.11 (s, 3H), 2.07 (s, 3H), 2.06-1.98 (m, 1H), 1.71-1.53 (m, 1H). [M+H] 482.0.

### Example 155

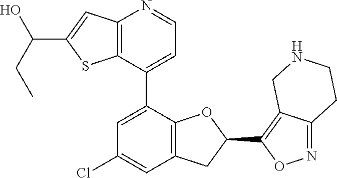 [SMILES: CCC(O)c1cc2nccc(-c3cc(Cl)cc4c3O[C@@H](c3onc5c3CNCC5)C4)c2s1]

### 1-(7-((R)-5-chloro-2-(4,5,6,7-tetrahydroisoxazolo[4,3-c]pyridin-3-yl)-2,3-dihydrobenzofuran-7-yl)thieno[3,2-b]pyridin-2-yl)propan-1-ol

The title compound was synthesized using precursor III, general procedure C, E and F using 1-(7-chlorothieno[3,2-b]pyridin-2-yl)propan-1-ol to give the desired product. 1H NMR (400 MHz, Methanol-d) δ 8.61 (d, J=5.0 Hz, 1H), 7.52 (t, J=2.6 Hz, 1H), 7.47-7.45 (m, 1H), 7.43 (dd, J=5.0, 1.9 Hz, 1H), 7.40-7.38 (m, 1H), 6.10 (dd, J=9.9, 6.2 Hz, 1H), 4.93 (q, J=6.1 Hz, 1H), 3.82 (dd, J=16.4, 9.9 Hz, 1H), 3.66 (d, J=7.6 Hz, 2H), 3.62-3.50 (m, 1H), 3.01 (t, J=6.2 Hz, 2H), 2.77 (t, J=6.0 Hz, 2H), 1.94-1.83 (m, 1H), 1.01 (t, J=7.4 Hz, 3H). [M+H] 468.0.

### Example 156

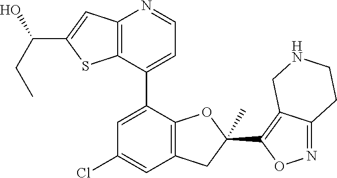 [SMILES: CC[C@H](O)c1cc2nccc(-c3cc(Cl)cc4c3O[C@@](C)(c3onc5c3CNCC5)C4)c2s1]

### (S)-1-(7-((R)-5-chloro-2-methyl-2-(4,5,6,7-tetrahydroisoxazolo[4,3-c]pyridin-3-yl)-2,3-dihydrobenzofuran-7-yl)thieno[3,2-b]pyridin-2-yl)propan-1-ol

The intermediate 7-bromo-5-chloro-2-methyl-2,3-dihydrobenzofuran-2-carboxylic acid was prepared from 2-bromo-4-chloro-phenol and 3-bromo-2-methylprop-1-ene (Example 91) as in steps 1-5 for precursor III. The title compound was synthesized using general procedure C and D using 1-(7-chlorothieno[3,2-b]pyridin-2-yl)propan-1-ol to give the desired product. 1H NMR (400 MHz, Methanol-d) δ 8.64 (d, J=4.9 Hz, 1H), 7.51 (d, J=2.2 Hz, 1H), 7.46 (d, J=5.0 Hz, 1H), 7.41 (d, J=1.0 Hz, 2H), 4.93 (t, J=6.6 Hz, 1H), 3.77-3.67 (m, 1H), 3.54 (d, J=16.2 Hz, 2H), 3.51-3.43 (m, 1H), 2.89 (t, J=6.2 Hz, 2H), 2.70 (t, J=5.9 Hz, 2H), 1.94-1.82 (m, 5H), 1.02 (t, J=7.4 Hz, 3H).

### Example 157

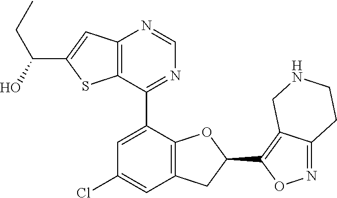 [SMILES: CC[C@@H](O)c1cc2ncnc(-c3cc(Cl)cc4c3O[C@@H](c3onc5c3CNCC5)C4)c2s1]

### (R)-1-(4-((R)-5-chloro-2-(4,5,6,7-tetrahydroisoxazolo[4,3-c]pyridin-3-yl)-2,3-dihydrobenzofuran-7-yl)thieno[3,2-d]pyrimidin-6-yl)propan-1-ol

The title compound was synthesized using precursor III, general procedure G and D using 1-(4-chlorothieno[3,2-d]pyrimidin-6-yl)propan-1-ol to give the desired product as the HCl salt. 1H NMR (400 MHz, CD₃OD): δ ppm 9.09 (s, 1H), 7.63 (s, 1H), 7.55 (s, 1H), 7.39 (s, 1), 6.09 (bs, 1H), 4.98 (q, J=6.9 Hz, 1H), 4.45-4.21 (m, 2H), 3.96-3.55 (m, 4H), 2.79 (t, J=5.2 Hz, 2H), 1.98-1.89 (m, 2H), 0.98 (t, J=6.2 Hz, 3H). [M+H] 469.1.

### Example 158

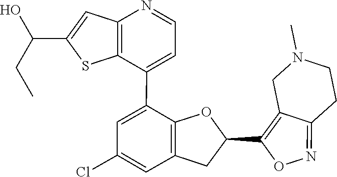 [SMILES: CCC(O)c1cc2nccc(-c3cc(Cl)cc4c3O[C@@H](c3onc5c3CN(C)CC5)C4)c2s1]

### 1-(7-((R)-5-chloro-2-(5-methyl-4,5,6,7-tetrahydroisoxazolo[4,3-c]pyridin-3-yl)-2,3-dihydrobenzofuran-7-yl)thieno[3,2-b]pyridin-2-yl)propan-1-ol

The title compound was synthesized using precursor III, general procedure C and D using 1-(7-chlorothieno[3,2-b]pyridin-2-yl)propan-1-ol to give the desired product as the HCl salt. 1H NMR (400 MHz, CD₃OD): δ ppm 8.63 (d, J=5.0 Hz, 1H), 7.55 (d, J=9.4 Hz, 1H), 7.51-7.38 (m, 2H), 7.41 (s, 1H), 6.13 (d, J=4.6 Hz, 1H), 4.93 (t, 1H), 3.90-3.75 (m, 1H), 3.60-3.42 (m, 1H), 3.15-2.95 (m, 2H), 2.78 (t, J=5.0 Hz, 2H), 2.63 (d, J=6.0 Hz, 2H), 2.11 (s, 1H), 2.08 (s, 1H), 1.91-1.81 (m, 1H), 1.28 (s, 1H), 1.00 (td, J=7.4, 3.9 Hz, 3H).

### Example 159

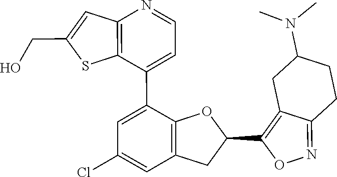 [SMILES: CN(C)C1CCc2noc([C@H]3Cc4cc(Cl)cc(-c5ccnc6cc(CO)sc56)c4O3)c2C1]

### (7-((2R)-5-chloro-2-(5-(dimethylamino)-4,5,6,7-tetrahydrobenzo[c]isoxazol-3-yl)-2,3-dihydrobenzofuran-7-yl)thieno[3,2-b]pyridin-2-yl)methanol

The title compound was synthesized using precursor V, general procedure C using 7-chloro-2-((2,2-dimethyl-1,1-diphenylpropoxy)methyl)thieno[3,2-b]pyridine. The product was disilylated with TBAF to afford the desired product. 1H NMR (400 MHz, CD₃OD): δ ppm 8.81 (dd, J=5.9, 1.5 Hz, 1H), 7.87 (dd, J=5.9, 2.0 Hz, 1H), 7.67-7.61 (m, 2H), 7.57 (dq, J=1.9, 1.0 Hz, 1H), 6.16 (ddd, J=9.9, 6.9, 2.2 Hz, 1H), 5.01 (s, 2H), 3.87-3.77 (m, 1H), 3.71-3.56 (m, 1H), 3.15-3.01 (m, 2H), 2.87 (s, 3H), 2.85 (s, 3H), 2.84-2.66 (m, 3H), 2.40-2.30 (m, 1H), 1.94 (qdd, J=12.5, 5.4, 1.8 Hz, 1H). [M+H] 482.0.

### Example 160

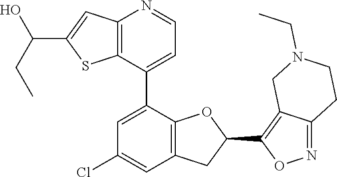 [SMILES: CCC(O)c1cc2nccc(-c3cc(Cl)cc4c3O[C@@H](c3onc5c3CN(CC)CC5)C4)c2s1]

### 1-(7-((R)-5-chloro-2-(5-ethyl-4,5,6,7-tetrahydroisoxazolo[4,3-c]pyridin-3-yl)-2,3-dihydrobenzofuran-7-yl)thieno[3,2-b]pyridin-2-yl)propan-1-ol

The title compound was synthesized from reductive amination of the title compound from Example 155 using acetaldehyde and sodium triacetoxyborohydride to give the desired product. 1H NMR (400 MHz, Chloroform-d) δ 8.70 (d, J=5.0 Hz, 1H), 7.59-7.21 (m, 3H), 6.11-5.90 (m, 1H), 3.89-3.70 (m, 1H), 2.08-1.43 (m, 12H), 1.34-1.14 (m, 2H), 1.02 (dt, J=17.7, 7.3 Hz, 4H). [M+H] 496.0.

### Example 161

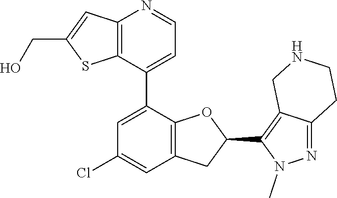 [SMILES: Cn1nc2c(c1[C@H]1Cc3cc(Cl)cc(-c4ccnc5cc(CO)sc45)c3O1)CNCC2]

### (R)-(7-(5-chloro-2-(2-methyl-4,5,6,7-tetrahydro-2H-pyrazolo[4,3-c]pyridin-3-yl)-2,3-dihydrobenzofuran-7-yl)thieno[3,2-b]pyridin-2-yl)methanol

The title compound was synthesized using precursor IV, general procedure C and D using 7-chloro-2-(((trimethylsilyl)oxy)methyl)thieno[3,2-b]pyridine to give the desired product as the HCl salt. 1H NMR (400 MHz, CD₃OD) δ 8.74 (d, J=6.0 Hz, 1H), 7.84 (d, J=6.0 Hz, 1H), 7.56-7.52 (m, 1H), 7.51 (d, J=2.1 Hz, 1H), 7.48-7.39 (m, 1H), 5.91 (t, J=8.5 Hz, 1H), 4.93 (d, J=1.2 Hz, 2H), 3.96 (q, J=14.5 Hz, 2H), 3.67-3.54 (m, 5H), 3.36 (td, J=6.4, 2.9 Hz, 2H), 2.89 (t, J=6.2 Hz, 2H). [M+H] 437.1.

### Example 162

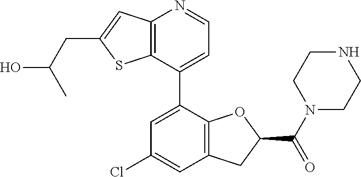 [SMILES: CC(O)Cc1cc2nccc(-c3cc(Cl)cc4c3O[C@@H](C(=O)N3CCNCC3)C4)c2s1]

### ((2R)-5-chloro-7-(2-(2-hydroxypropyl)thieno[3,2-b]pyridin-7-yl)-2,3-dihydrobenzofuran-2-yl)(piperazin-1-yl)methanone

The title compound was synthesized using precursor II, general procedure B and D using 1-(7-chlorothieno[3,2-b]pyridin-2-yl)propan-2-ol to give the desired product as the HCl salt. 1H NMR (400 MHz, Methanol-d4) δ 8.86 (bs, 1H), 8.11 (d, J=6.0 Hz, 1H), 7.69 (dd, J=2.2, 0.8 Hz, 1H), 7.64 (s, 1H), 7.54 (dt, J=2.2, 1.2 Hz, 1H), 5.90 (t, J=7.9 Hz, 1H), 4.19-4.09 (m, 1H), 3.92-3.80 (m, 4H), 3.68 (d, J=8.2 Hz, 2H), 3.37-3.11 (m, 6H), 1.30 (dd, J=6.2, 2.0 Hz, 3H). [M+H] 458.0.

### Example 163

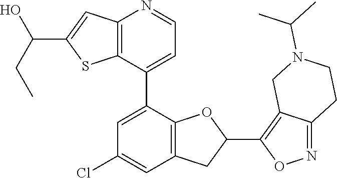 [SMILES: CCC(O)c1cc2nccc(-c3cc(Cl)cc4c3OC(c3onc5c3CN(C(C)C)CC5)C4)c2s1]

### 1-(7-(5-chloro-2-(5-isopropyl-4,5,6,7-tetrahydroisoxazolo[4,3-c]pyridin-3-yl)-2,3-dihydrobenzofuran-7-yl)thieno[3,2-b]pyridin-2-yl)propan-1-ol

The title compound was synthesized from reductive amination of the title compound from Example 155 using acetone and sodium triacetoxyborohydride to give the desired product. 1H NMR (400 MHz, Chloroform-d) δ 8.70 (dd, J=4.8, 2.3 Hz, 1H), 7.53-7.28 (m, 4H), 6.09-5.91 (m, 1H), 5.00 (d, J=8.6 Hz, 1H), 3.90-2.72 (m, 6H), 2.05-1.38 (m, 8H), 1.34-1.19 (m, 1H), 1.00 (tt, J=11.0, 5.9 Hz, 5H). [M+H] 510.0.

### Example 164

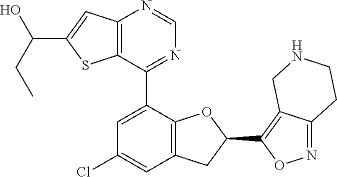 [SMILES: CCC(O)c1cc2ncnc(-c3cc(Cl)cc4c3O[C@@H](c3onc5c3CNCC5)C4)c2s1]

### 1-(4-((R)-5-chloro-2-(4,5,6,7-tetrahydroisoxazolo[4,3-c]pyridin-3-yl)-2,3-dihydrobenzofuran-7-yl)thieno[3,2-d]pyrimidin-6-yl)propan-1-ol

The title compound was synthesized using precursor III, general procedure G and D using 1-(4-chlorothieno[3,2-d]pyrimidin-6-yl)propan-1-ol to give the desired product as the HCl salt. 1H NMR (400 MHz, CD3OD): δ ppm 9.09 (s, 1H), 7.63 (s, 1H), 7.55 (s, 1H), 7.39 (s, 1), 6.09 (bs, 1H), 4.98 (q, J=6.9 Hz, 1H), 4.45-4.21 (m, 2H), 3.96-3.55 (m, 4H), 2.79 (t, J=5.2 Hz, 2H), 1.98-1.89 (m, 2H), 0.98 (t, J=6.2 Hz, 3H). [M+H] 469.1.

### Example 165

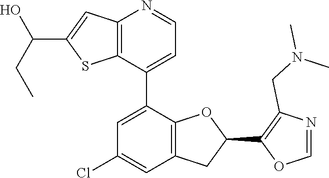 [SMILES: CCC(O)c1cc2nccc(-c3cc(Cl)cc4c3O[C@@H](c3ocnc3CN(C)C)C4)c2s1]

### 1-(7-((R)-5-chloro-2-(4-((dimethylamino)methyl)oxazol-5-yl)-2,3-dihydrobenzofuran-7-yl)thieno[3,2-b]pyridin-2-yl)propan-1-ol

The title compound was synthesized using precursor VII, general procedure C, E and F using 1-(7-(4,4,5,5-tetramethyl-1,3,2-dioxaborolan-2-yl)thieno[3,2-b]pyridin-2-yl)propan-1-ol to give the desired product. 1H NMR (400 MHz, Methanol-d) δ 8.57 (dd, J=5.0, 1.4 Hz, 1H), 8.16 (d, J=5.1 Hz, 1H), 7.49-7.47 (m, 1H), 7.44 (dt, J=2.3, 1.1 Hz, 1H), 7.38 (dd, J=5.0, 0.6 Hz, 1H), 7.37 (d, J=1.0 Hz, 1H), 6.16 (dd, J=9.8, 7.4 Hz, 1H), 4.94-4.89 (m, 2H), 3.73 (dd, J=16.3, 9.8 Hz, 1H), 3.60 (dd, J=16.3, 7.5 Hz, 1H), 3.54-3.37 (m, 2H), 2.18 (d, J=0.6 Hz, 6H), 1.93-1.76 (m, 2H), 0.99 (dt, J=9.6, 7.4 Hz, 3H). [M+H] 470.0.

### Example 166

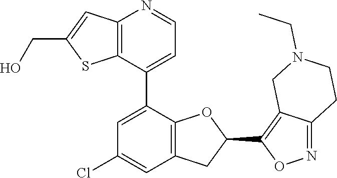 [SMILES: CCN1CCc2noc([C@H]3Cc4cc(Cl)cc(-c5ccnc6cc(CO)sc56)c4O3)c2C1]

### (R)-(7-(5-chloro-2-(5-ethyl-4,5,6,7-tetrahydroisoxazolo[4,3-c]pyridin-3-yl)-2,3-dihydrobenzofuran-7-yl)thieno[3,2-b]pyridin-2-yl)methanol

The title compound was synthesized from reductive amination of the intermediate from Example 116 using acetaldehyde and sodium triacetoxyborohydride to give the desired product. 1H NMR (400 MHz, Chloroform-d) δ 8.69 (d, J=4.8 Hz, 1H), 7.39 (s, 1H), 7.37-7.32 (m, 2H), 7.25 (d, J=4.8 Hz, 1H), 5.92 (dd, J=9.6, 6.1 Hz, 1H), 5.05-4.82 (m, 2H), 3.75 (qd, J=16.2, 7.8 Hz, 2H), 3.40 (d, J=14.6 Hz, 1H), 3.24 (t, J=15.7 Hz, 1H), 3.09-2.88 (m, 3H), 2.48 (hept, J=6.2 Hz, 2H), 1.30-1.21 (m, 1H), 0.99 (t, J=7.2 Hz, 3H). [M+H] 468.0.

### Example 167

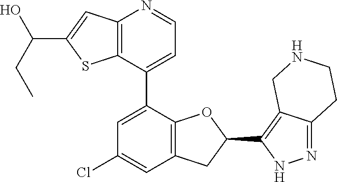 [SMILES: CCC(O)c1cc2nccc(-c3cc(Cl)cc4c3O[C@@H](c3[nH]nc5c3CNCC5)C4)c2s1]

### 1-(7-((R)-5-chloro-2-(4,5,6,7-tetrahydro-2H-pyrazolo[4,3-c]pyridin-3-yl)-2,3-dihydrobenzofuran-7-yl)thieno[3,2-b]pyridin-2-yl)propan-1-ol

The title compound was synthesized using precursor IV, general procedure C and D using 1-(7-chlorothieno[3,2-b]pyridin-2-yl)propan-1-ol to give the desired product as the HCl salt. 1H NMR (400 MHz, CD3OD): δ ppm 8.88 (d, J=5.2 Hz, 1H), 8.00 (d, J=5.2 Hz, 1H), 7.67 (s, 1H), 7.63 (s, 1H), 7.59 (s, 1H), 6.08 (t, J=8.4 Hz, 1H), 5.10 (bs, 1H), 4.08 (s, 2H), 3.80-3.56 (m, 4H), 1.97-1.86 (m, 2H), 1.03 (t, J=7.6 Hz, 3H). [M+H] 467.0.

### Example 168

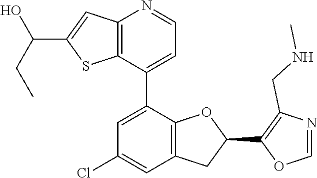 [SMILES: CCC(O)c1cc2nccc(-c3cc(Cl)cc4c3O[C@@H](c3ocnc3CNC)C4)c2s1]

### 1-(7-((R)-5-chloro-2-(4-((methylamino)methyl)oxazol-5-yl)-2,3-dihydrobenzofuran-7-yl)thieno[3,2-b]pyridin-2-yl)propan-1-ol

The title compound was synthesized using precursor VII, general procedure C, E and F using 1-(7-(4,4,5,5-tetramethyl-1,3,2-dioxaborolan-2-yl)thieno[3,2-b]pyridin-2-yl)propan-1-ol to give the desired product. 1H NMR (400 MHz, Methanol-d) δ 8.57 (dd, J=5.0, 0.9 Hz, 1H), 8.16 (d, J=4.3 Hz, 1H), 7.51-7.45 (m, 1H), 7.47-7.42 (m, 1H), 7.39 (d, J=5.0 Hz, 1H), 7.38-7.36 (m, 1H), 6.15 (dd, J=9.7, 7.5 Hz, 1H), 4.94-4.89 (m, 1H), 3.74 (dd, J=16.4, 9.8 Hz, 1H), 3.64 (d, J=2.1 Hz, 2H), 3.59 (dd, J=16.5, 7.2 Hz, 1H), 2.25 (s, 3H), 1.86 (h, J=7.3 Hz, 2H), 0.99 (q, J=7.6 Hz, 3H). [M+H] 456.0.

### Example 169

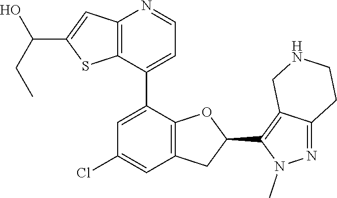 [SMILES: CCC(O)c1cc2nccc(-c3cc(Cl)cc4c3O[C@@H](c3c5c(nn3C)CCNC5)C4)c2s1]

### 1-(7-((R)-5-chloro-2-(2-methyl-4,5,6,7-tetrahydro-2H-pyrazolo[4,3-c]pyridin-3-yl)-2,3-dihydrobenzofuran-7-yl)thieno[3,2-b]pyridin-2-yl)propan-1-ol

The title compound was synthesized using precursor IV, general procedure C and D using 1-(7-chlorothieno[3,2-b]pyridin-2-yl)propan-1-ol to give the desired product as the HCl salt. [M+H] 481.0.

### Example 170

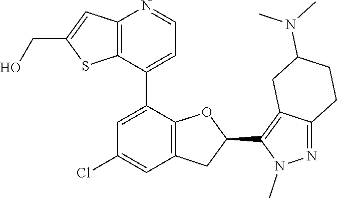 [SMILES: CN(C)C1CCc2nn(C)c([C@H]3Cc4cc(Cl)cc(-c5ccnc6cc(CO)sc56)c4O3)c2C1]

### (7-((2R)-5-chloro-2-(5-(dimethylamino)-2-methyl-4,5,6,7-tetrahydro-2H-indazol-3-yl)-2,3-dihydrobenzofuran-7-yl)thieno[3,2-b]pyridin-2-yl)methanol

The product of Step 4, Precursor V was subjected to general procedure C using 7-(4,4,5,5-tetramethyl-1,3,2-dioxaborolan-2-yl)-2-(((2,3,3-trimethylbutan-2-yl)oxy)methyl)thieno[3,2-b]pyridine. The title compound was synthesized by reductive amination using dimethylamine and sodium triacetoxyborohydride to give the desired product. 1H NMR (400 MHz, CD3OD) δ 8.44 (dd, J=5.0, 3.6 Hz, 1H), 7.34 (dd, J=7.3, 2.2 Hz, 1H), 7.30 (t, J=4.9 Hz, 1H), 7.27 (ddt, J=6.0, 2.5, 1.2 Hz, 2H), 5.76 (ddd, J=9.4, 7.7, 6.2 Hz, 1H), 4.76 (t, J=1.4 Hz, 2H), 3.57 (d, J=17.7 Hz, 1H), 3.52 (s, 3H), 3.51-3.39 (m, 1H), 2.72-2.51 (m, 1H), 2.41 (dd, J=16.7, 11.4 Hz, 3H), 2.24-2.07 (m, 1H), 2.04 (d, J=4.1 Hz, 6H), 1.94 (s, 1H), 1.62-1.42 (m, 1H). [M+H] 495.2.

### Example 171

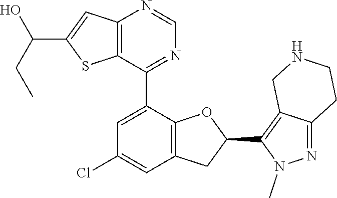 [SMILES: CCC(O)c1cc2ncnc(-c3cc(Cl)cc4c3O[C@@H](c3c5c(nn3C)CCNC5)C4)c2s1]

### 1-(4-((R)-5-chloro-2-(2-methyl-4,5,6,7-tetrahydro-2H-pyrazolo[4,3-c]pyridin-3-yl)-2,3-dihydrobenzofuran-7-yl)thieno[3,2-d]pyrimidin-6-yl)propan-1-ol

The title compound was synthesized using precursor IV, general procedure C and D using 1-(4-chlorothieno[3,2-d]pyrimidin-6-yl)propan-1-ol to give the desired product as the HCl salt. 1H NMR (400 MHz, CD3OD) δ 9.16 (s, 1H), 7.59 (dd, J=6.2, 2.1 Hz, 1H), 7.49 (s, 1H), 7.46-7.43 (m, 1H), 5.97 (td, J=8.3, 3.8 Hz, 1H), 4.97 (dd, J=7.6, 4.6 Hz, 1H), 4.03 (s, 2H), 3.64 (s, 3H), 3.61 (ddd, J=6.0, 4.5, 1.1 Hz, 2H), 3.54 (s, 1H), 3.48-3.43 (m, 1H), 3.37 (q, J=6.2 Hz, 2H), 2.89 (t, J=6.1 Hz, 2H), 0.91 (td, J=7.4, 5.4 Hz, 3H). [M+H] 482.1.

### Example 172

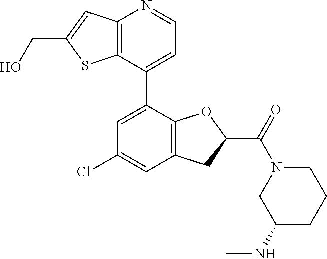 [SMILES: CN[C@H]1CCCN(C(=O)[C@H]2Cc3cc(Cl)cc(-c4ccnc5cc(CO)sc45)c3O2)C1]

### ((R)-5-chloro-7-(2-(hydroxymethyl)thieno[3,2-b]pyridin-7-yl)-2,3-dihydrobenzofuran-2-yl)((S)-3-(methylamino)piperidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.67 (d, J=4.8 Hz, 1H), 7.49-7.00 (m, 4H), 5.72-5.34 (m, 1H), 5.12-4.82 (m, 2H), 4.72-3.72 (m, 3H), 3.63-2.49 (m, 5H), 2.43 (s, 2H), 2.14-0.79 (m, 4H). [M+H] 458.0.

### Example 173

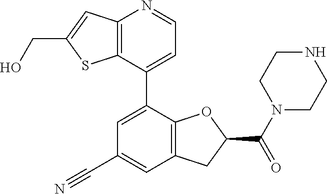 [SMILES: N#Cc1cc2c(c(-c3ccnc4cc(CO)sc34)c1)O[C@@H](C(=O)N1CCNCC1)C2]

### (R)-7-(2-(hydroxymethyl)thieno[3,2-b]pyridin-7-yl)-2-(piperazine-1-carbonyl)-2,3-dihydrobenzofuran-5-carbonitrile

The title compound was synthesized using precursor VIII, general procedure C and D using (7-chlorothieno[3,2-b]pyridin-2-yl)methanol to give the desired compound as the HCl salt. 1H NMR (400 MHz, CD3OD): δ ppm 8.63 (d, J=5.2 Hz, 1H), 7.86 (s, 1H), 7.76 (s, 1H), 7.51 (d, J=5.2 Hz, 1H), 7.43 (s, 1H), 5.840 (dd, J=10.0, 6.8 Hz, 1H), 4.92 (s, 2H), 3.82-3.40 (m, 6H), 2.81-2.51 (m, 4H). [M+H] 421.0.

### Example 174

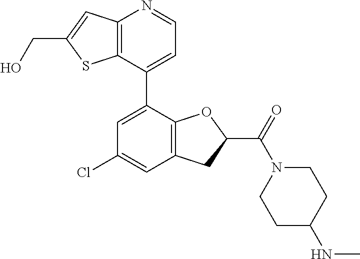 [SMILES: CNC1CCN(C(=O)[C@H]2Cc3cc(Cl)cc(-c4ccnc5cc(CO)sc45)c3O2)CC1]

### (R)-(5-chloro-7-(2-(hydroxymethyl)thieno[3,2-b]pyridin-7-yl)-2,3-dihydrobenzofuran-2-yl)(4-(methylamino)piperidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. [M+H] 458.0.

### Example 175

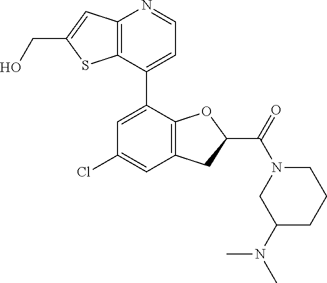 [SMILES: CN(C)C1CCCN(C(=O)[C@H]2Cc3cc(Cl)cc(-c4ccnc5cc(CO)sc45)c3O2)C1]

### ((R)-5-chloro-7-(2-(hydroxymethyl)thieno[3,2-b]pyridin-7-yl)-2,3-dihydrobenzofuran-2-yl)(3-(dimethylamino)piperidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.70 (dd, J=12.7, 4.7 Hz, 1H), 7.49-7.14 (m, 4H), 5.61-5.40 (m, 1H), 5.16-4.48 (m, 4H), 4.30-3.73 (m, 2H), 3.54-2.95 (m, 2H), 2.94-0.73 (m, 11H). [M+H] 472.0.

### Example 176

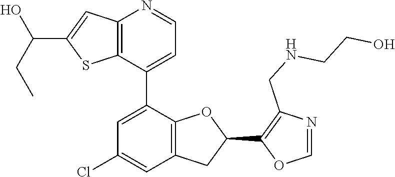 [SMILES: CCC(O)c1cc2nccc(-c3cc(Cl)cc4c3O[C@@H](c3ocnc3CNCCO)C4)c2s1]

### 1-(7-((R)-5-chloro-2-(4-(((2-hydroxyethyl)amino)methyl)oxazol-5-yl)-2,3-dihydrobenzofuran-7-yl)thieno[3,2-b]pyridin-2-yl)propan-1-ol

The title compound was synthesized using precursor VII, general procedure C, E and F using 1-(7-(4,4,5,5-tetramethyl-1,3,2-dioxaborolan-2-yl)thieno[3,2-b]pyridin-2-yl)propan-1-ol to give the desired product. 1H NMR (400 MHz, Chloroform-d) δ 8.58 (dd, J=4.9, 0.9 Hz, 1H), 8.16 (d, J=3.0 Hz, 1H), 7.51-7.45 (m, 1H), 7.47-7.42 (m, 1H), 7.39 (d, J=4.9 Hz, 1H), 7.37 (d, J=0.9 Hz, 1H), 6.17 (ddd, J=9.6, 7.4, 1.8 Hz, 1H), 4.93-4.89 (m, 1H), 3.78-3.69 (m, 3H), 3.60 (dd, J=16.0, 7.3 Hz, 1H), 3.56-3.50 (m, 4H), 2.62-2.54 (m, 1H), 1.86 (dq, J=14.2, 7.4 Hz, 2H), 0.99 (q, J=7.3 Hz, 2H). [M+H] 486.0.

### Example 177

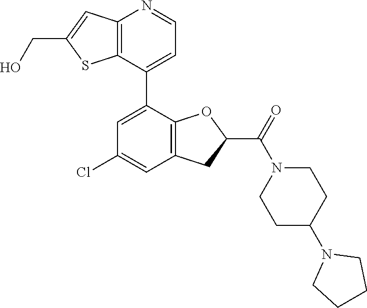 [SMILES: O=C([C@H]1Cc2cc(Cl)cc(-c3ccnc4cc(CO)sc34)c2O1)N1CCC(N2CCCC2)CC1]

### (R)-(5-chloro-7-(2-(hydroxymethyl)thieno[3,2-b]pyridin-7-yl)-2,3-dihydrobenzofuran-2-yl)(4-(pyrrolidin-1-yl)piperidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.69 (dd, J=29.9, 5.0 Hz, 1H), 7.66-7.48 (m, 1H), 7.39-7.27 (m, 3H), 5.53 (q, J=8.2, 7.3 Hz, 1H), 5.11-4.83 (m, 2H), 4.68 (t, J=15.5 Hz, 1H), 4.30 (d, J=13.7 Hz, 1H), 3.98 (td, J=16.2, 7.7 Hz, 1H), 3.78-2.52 (m, 5H), 2.02 (s, 2H), 1.90 (d, J=11.3 Hz, 1H), 1.78 (s, 8H). [M+H] 498.0.

### Example 178

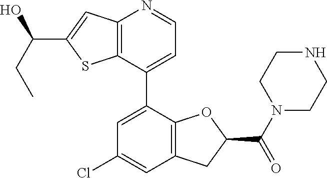 [SMILES: CC[C@@H](O)c1cc2nccc(-c3cc(Cl)cc4c3O[C@@H](C(=O)N3CCNCC3)C4)c2s1]

### ((R)-5-chloro-7-(2-((R)-1-hydroxypropyl)thieno[3,2-b]pyridin-7-yl)-2,3-dihydrobenzofuran-2-yl)(piperazin-1-yl)methanone

The title compound was synthesized using precursor II, general procedure B and D using (R)-1-(7-chlorothieno[3,2-b]pyridin-2-yl)propan-1-ol to give the compound as the HCl salt. 1H NMR (400 MHz, Methanol-d4) δ 8.90 (d, J=5.5 Hz, 1H), 8.12 (d, J=5.7 Hz, 1H), 7.67 (d, J=2.4 Hz, 1H), 7.62 (d, J=7.1 Hz, 1H), 7.55 (d, J=1.6 Hz, 1H), 5.91 (t, J=8.0 Hz, 1H), 5.13 (dd, J=7.5, 4.9 Hz, 1H), 3.96-3.54 (m, 6H), 3.28-3.11 (m, 4H), 2.04-1.84 (m, 2H), 1.05 (t, J=7.3 Hz, 3H). [M+H] 458.0.

### Example 179

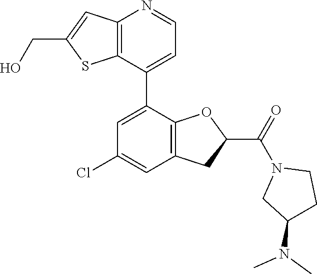 [SMILES: CN(C)[C@@H]1CCN(C(=O)[C@H]2Cc3cc(Cl)cc(-c4ccnc5cc(CO)sc45)c3O2)C1]

### ((R)-5-chloro-7-(2-(hydroxymethyl)thieno[3,2-b]pyridin-7-yl)-2,3-dihydrobenzofuran-2-yl)((R)-3-(dimethylamino)pyrrolidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.68 (dd, J=4.8, 3.1 Hz, 1H), 7.44-7.10 (m, 4H), 5.55-5.25 (m, 1H), 5.09-4.85 (m, 2H), 4.25-3.58 (m, 4H), 3.55-3.26 (m, 2H), 2.50 (s, 6H), 2.31-1.27 (m, 3H). [M+H] 458.0.

### Example 180

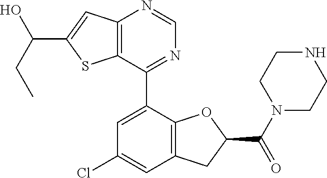 [SMILES: CCC(O)c1cc2ncnc(-c3cc(Cl)cc4c3O[C@@H](C(=O)N3CCNCC3)C4)c2s1]

### ((2R)-5-chloro-7-(6-(1-hydroxypropyl)thieno[3,2-d]pyrimidin-4-yl)-2,3-dihydrobenzofuran-2-yl)(piperazin-1-yl)methanone

The title compound was synthesized using precursor II, general procedure B and D using 1-(4-chlorothieno[3,2-d]pyrimidin-6-yl)propan-1-ol to give the desired compound as the HCl salt. 1H NMR (400 MHz, CD₃OD): δ ppm 9.12 (s, 1H), 7.56 (dd, J=5.6, 2.4 Hz, 1H), 7.47 (s, 1H), 7.42 (d, J=5.6 Hz, 1H), 5.81 (dd, J=9.6, 7.2 Hz, 1H), 5.02 (quin, J=6.8 Hz, 1H), 3.71-1.90 (m, 11H), 1.95-1.85 (m, 2H), 1.04 (t, J=7.2 Hz, 3H). [M+H] 459.0.

### Example 181

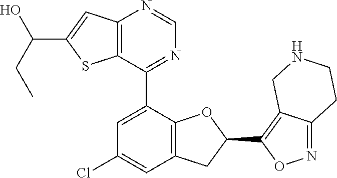 [SMILES: CCC(O)c1cc2ncnc(-c3cc(Cl)cc4c3O[C@@H](c3onc5c3CNCC5)C4)c2s1]

### 1-(4-((R)-5-chloro-2-(4,5,6,7-tetrahydroisoxazolo[4,3-c]pyridin-3-yl)-2,3-dihydrobenzofuran-7-yl)thieno[3,2-d]pyrimidin-6-yl)propan-1-ol

The title compound was synthesized using precursor III, general procedure B and D using 1-(4-chlorothieno[3,2-d]pyrimidin-6-yl)propan-1-ol to give the desired compound as the HCl salt. 1H NMR (400 MHz, CD3OD): δ ppm 9.28 (s, 1H), 7.73-7.70 (m, 1H), 7.62 (s, 1H), 7.53 (s, 1H), 6.28 (dd, J=9.6, 6.4 Hz, 1H), 5.09-5.05 (m, 1H), 4.30 (s, 2H), 3.91-3.47 (m, 4H), 3.12 (t, J=6.4 Hz, 2H), 1.96-1.84 (m, 2H), 1.02 (t, J=7.6 Hz, 3H). [M+H] 469.0.

### Example 182

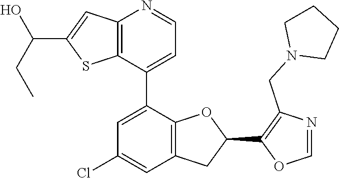 [SMILES: CCC(O)c1cc2nccc(-c3cc(Cl)cc4c3O[C@@H](c3ocnc3CN3CCCC3)C4)c2s1]

### 1-(7-((R)-5-chloro-2-(4-(pyrrolidin-1-ylmethyl)oxazol-5-yl)-2,3-dihydrobenzofuran-7-yl)thieno[3,2-b]pyridin-2-yl)propan-1-ol

The title compound was synthesized using precursor VII, general procedure C, E and F using 1-(7-(4,4,5,5-tetramethyl-1,3,2-dioxaborolan-2-yl)thieno[3,2-b]pyridin-2-yl)propan-1-ol to give the desired product. 1H NMR (400 MHz, Methanol-d) δ 8.59-8.55 (m, 1H), 8.14 (d, J=4.7 Hz, 1H), 7.51-7.45 (m, 1H), 7.47-7.41 (m, 1H), 7.39 (dd, J=5.0, 1.1 Hz, 1H), 7.37 (d, J=0.9 Hz, 1H), 6.18 (dd, J=9.9, 7.4 Hz, 1H), 3.74 (dd, J=16.1, 10.2 Hz, 1H), 3.67-3.50 (m, 2H), 2.53-2.43 (m, 4H), 1.91-1.79 (m, 2H), 1.74-1.63 (m, 6H), 1.05-0.93 (m, 3H). [M+H] 496.0.

### Example 183

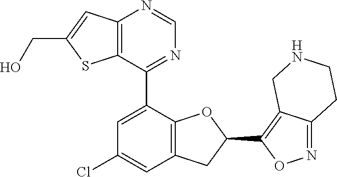 [SMILES: OCc1cc2ncnc(-c3cc(Cl)cc4c3O[C@@H](c3onc5c3CNCC5)C4)c2s1]

### (R)-(4-(5-chloro-2-(4,5,6,7-tetrahydroisoxazolo[4,3-c]pyridin-3-yl)-2,3-dihydrobenzofuran-7-yl)thieno[3,2-d]pyrimidin-6-yl)methanol

The title compound was synthesized using precursor III, general procedure G and D using (4-chlorothieno[3,2-d]pyrimidin-6-yl)methanol to give the desired product as the HCl salt. 1H NMR (400 MHz, CD3OD) δ 9.22 (s, 1H), 7.69 (s, 1H), 7.58 (s, 1H), 7.49 (s, 1H), 6.25 (s, 1H), 5.00 (s, 2H), 4.27 (s, 2H), 4.18-4.03 (m, 1H), 3.95-3.79 (m, 1H), 3.63 (d, J=2.0 Hz, 1H), 3.48 (s, 1H), 3.10 (s, 2H). [M+H] 441.1.

### Example 184

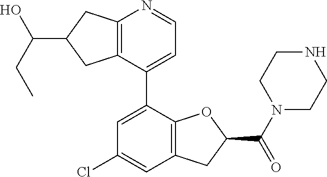 [SMILES: CCC(O)C1Cc2nccc(-c3cc(Cl)cc4c3O[C@@H](C(=O)N3CCNCC3)C4)c2C1]

### ((2R)-5-chloro-7-(6-(1-hydroxypropyl)-6,7-dihydro-5H-cyclopenta[b]pyridin-4-yl)-2,3-dihydrobenzofuran-2-yl)(piperazin-1-yl)methanone

The title compound was synthesized using precursor II, general procedure C, E and F using 1-(4-chloro-6,7-dihydro-5H-cyclopenta[b]pyridin-6-yl)propan-1-ol to give the desired product. 1H NMR (400 MHz, Methanol-d) δ 8.27 (d, J=5.3 Hz, 1H), 7.32 (s, 1H), 7.30-7.22 (m, 1H), 7.22-7.16 (m, 1H), 5.78-5.67 (m, 1H), 3.82-3.45 (m, 7H), 3.11-2.80 (m, 8H), 2.64-2.49 (m, 1H), 1.67-1.54 (m, 1H), 1.51-1.38 (m, 1H), 1.05-0.96 (m, 3H). [M+H] 442.1.

### Example 185

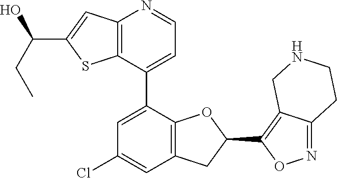 [SMILES: CC[C@@H](O)c1cc2nccc(-c3cc(Cl)cc4c3O[C@@H](c3onc5c3CNCC5)C4)c2s1]

### (R)-1-(7-((R)-5-chloro-2-(4,5,6,7-tetrahydroisoxazolo[4,3-c]pyridin-3-yl)-2,3-dihydrobenzofuran-7-yl)thieno[3,2-b]pyridin-2-yl)propan-1-ol

The title compound was synthesized using precursor III, general procedure C and E using 1-(thieno[3,2-b]pyridin-2-yl)propan-1-ol to give the desired product as the TFA salt. 1H NMR (400 MHz, Methanol-d) δ 8.61 (d, J=4.9 Hz, 1H), 7.52-7.49 (m, 1H), 7.47-7.45 (m, 1H), 7.43 (d, J=4.9 Hz, 1H), 7.39 (d, J=0.9 Hz, 1H), 6.09 (dd, J=9.9, 6.1 Hz, 1H), 4.94 (t, J=6.6 Hz, 1H), 3.81 (dd, J=16.4, 9.9 Hz, 1H), 3.60 (s, 2H), 3.60-3.51 (m, 1H), 2.96 (td, J=6.2, 3.0 Hz, 1H), 2.74 (t, J=6.0 Hz, 2H), 1.88 (p, J=7.3 Hz, 2H), 1.01 (t, J=7.4 Hz, 3H). [M+H] 468.0.

### Example 186

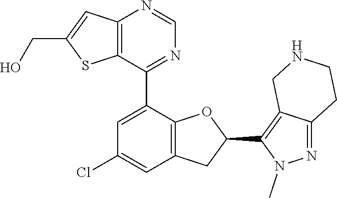 [SMILES: Cn1nc2c(c1[C@H]1Cc3cc(Cl)cc(-c4ncnc5cc(CO)sc45)c3O1)CNCC2]

### (R)-(4-(5-chloro-2-(2-methyl-4,5,6,7-tetrahydro-2H-pyrazolo[4,3-c]pyridin-3-yl)-2,3-dihydrobenzofuran-7-yl)thieno[3,2-d]pyrimidin-6-yl)methanol

The title compound was synthesized using precursor IV, general procedure G and D using (4-chlorothieno[3,2-d]pyrimidin-6-yl)methanol to give the desired product as the HCl salt. 1H NMR (400 MHz, CD3OD) δ 9.24 (s, 1H), 7.63 (d, J=2.1 Hz, 1H), 7.52 (d, J=4.5 Hz, 2H), 6.01 (t, J=8.5 Hz, 1H), 4.98 (s, 2H), 4.12-3.90 (m, 1H), 3.64 (s, 4H), 3.65-3.57 (m, 1H), 3.45 (dd, J=5.5, 4.1 Hz, 1H), 3.37 (q, J=8.1, 7.1 Hz, 2H), 2.90 (t, J=6.1 Hz, 2H). [M+H] 454.1.

### Example 187

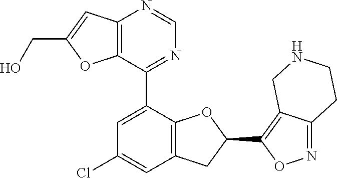 [SMILES: OCc1cc2ncnc(-c3cc(Cl)cc4c3O[C@@H](c3onc5c3CNCC5)C4)c2o1]

### (R)-(4-(5-chloro-2-(4,5,6,7-tetrahydroisoxazolo[4,3-c]pyridin-3-yl)-2,3-dihydrobenzofuran-7-yl)furo[3,2-d]pyrimidin-6-yl)methanol

The title compound was synthesized using precursor III, general procedure G and D using (4-chlorothieno[3,2-d]pyrimidin-6-yl)methanol to give the desired product as the HCl salt. 1H NMR (400 MHz, CD3OD) δ 9.04 (s, 1H), 7.77 (d, J=2.1 Hz, 1H), 7.50-7.44 (m, 1H), 6.99 (s, 1H), 6.14 (dd, J=10.0, 6.4 Hz, 1H), 4.22 (q, J=15.4 Hz, 2H), 3.76 (dd, J=16.4, 10.0 Hz, 1H), 3.53 (s, 6H), 3.50-3.31 (m, 3H), 3.00 (t, J=6.3 Hz, 2H). [M+H] 453.1.

### Example 188

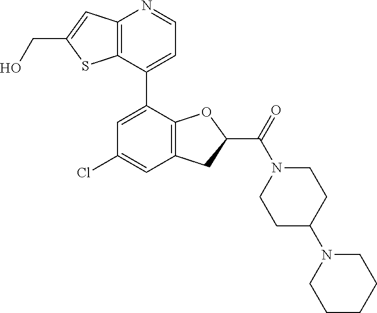 [SMILES: O=C([C@H]1Cc2cc(Cl)cc(-c3ccnc4cc(CO)sc34)c2O1)N1CCC(N2CCCCC2)CC1]

### (R)-[1,4′-bipiperidin]-1′-yl(5-chloro-7-(2-(hydroxymethyl)thieno[3,2-b]pyridin-7-yl)-2,3-dihydrobenzofuran-2-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.67 (d, J=5.1 Hz, 1H), 7.66 (s, 1H), 7.46-7.12 (m, 3H), 5.71-5.47 (m, 1H), 5.22-4.63 (m, 4H), 4.34 (d, J=13.8 Hz, 1H), 4.06-3.90 (m, 1H), 3.52-3.29 (m, 2H), 3.26-1.98 (m, 11H), 1.42-1.15 (m, 4H). [M+H] 512.2.

### Example 189

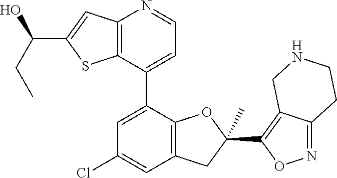 [SMILES: CC[C@@H](O)c1cc2nccc(-c3cc(Cl)cc4c3O[C@@](C)(c3onc5c3CNCC5)C4)c2s1]

### (R)-1-(7-((R)-5-chloro-2-methyl-2-(4,5,6,7-tetrahydroisoxazolo[4,3-c]pyridin-3-yl)-2,3-dihydrobenzofuran-7-yl)thieno[3,2-b]pyridin-2-yl)propan-1-ol

isomer of title compound from Example 156 obtained after chromatography. 1H NMR (400 MHz, Methanol-d) δ 8.64 (d, J=4.9 Hz, 1H), 7.50 (d, J=2.2 Hz, 1H), 7.45 (d, J=5.0 Hz, 1H), 7.42-7.40 (m, 2H), 4.95 (t, J=6.4 Hz, 1H), 3.74 (d, J=16.4 Hz, 1H), 3.58-3.44 (m, 3H), 2.88 (d, J=6.2 Hz, 2H), 2.70 (t, J=5.9 Hz, 2H), 1.97-1.84 (m, 5H), 1.02 (t, J=7.4 Hz, 3H).

### Example 190

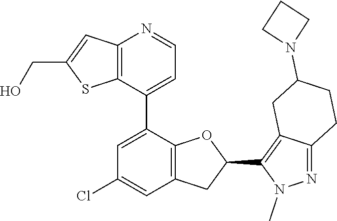 [SMILES: Cn1nc2c(c1[C@H]1Cc3cc(Cl)cc(-c4ccnc5cc(CO)sc45)c3O1)CC(N1CCC1)CC2]

### (7-((2R)-2-(5-(azetidin-1-yl)-2-methyl-4,5,6,7-tetrahydro-2H-indazol-3-yl)-5-chloro-2,3-dihydrobenzofuran-7-yl)thieno[3,2-b]pyridin-2-yl)methanol

The product of Step 4, Precursor V was subjected to general procedure C using 7-(4,4,5,5-tetramethyl-1,3,2-dioxaborolan-2-yl)-2-(((2,3,3-trimethylbutan-2-yl)oxy)methyl)thieno[3,2-b]pyridine. The title compound was synthesized by reductive amination using azetidine and sodium triacetoxyborohydride to give the desired product. 1H NMR (400 MHz, CD3OD) δ 8.46 (dd, J=5.0, 4.0 Hz, 1H), 7.39-7.18 (m, 4H), 5.78 (ddd, J=9.2, 7.9, 5.0 Hz, 1H), 4.82-4.76 (m, 2H), 4.03-3.87 (m, 1H), 3.65-3.55 (m, 1H), 3.54 (s, 3H), 3.46 (q, J=8.2, 7.5 Hz, 1H), 3.40-3.26 (m, 2H), 2.81 (s, 1H), 2.65-2.32 (m, 4H), 2.03 (t, J=7.4 Hz, 1H), 1.82 (d, J=17.8 Hz, 2H), 1.51 (t, J=10.4 Hz, 1H). [M+H] 507.0.

### Example 191

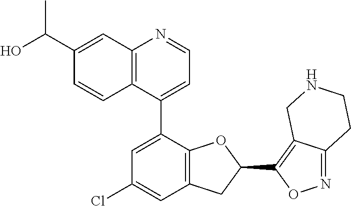 [SMILES: CC(O)c1ccc2c(-c3cc(Cl)cc4c3O[C@@H](c3onc5c3CNCC5)C4)ccnc2c1]

### 1-(4-((R)-5-chloro-2-(4,5,6,7-tetrahydroisoxazolo[4,3-c]pyridin-3-yl)-2,3-dihydrobenzofuran-7-yl)quinolin-7-yl)ethan-1-ol

The title compound was synthesized using precursor III, general procedure G and E using 1-(4-chloroquinolin-7-yl)ethan-1-ol to give the desired compound as the TFA salt. 1H NMR (400 MHz, CD3OD): δ ppm 8.20-8.10 (m, 2H), 7.95-7.79 (m, 2H), 7.71 (s, 1H), 7.64-7.48 (m, 1H), 7.41 (d, J=26.2 Hz, 1H), 6.20-6.00 (m, 1H), 5.09 (s, 1H), 3.99 (s, 1H), 3.88 (s, 1H), 3.46 (dd, J=23.0, 7.9 Hz, 2H), 3.19-2.95 (m, 4H), 2.03 (d, J=15.8 Hz, 3H).

### Example 192

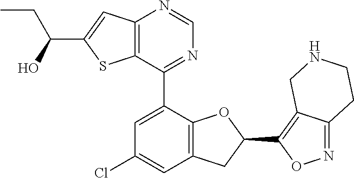 [SMILES: CC[C@H](O)c1cc2ncnc(-c3cc(Cl)cc4c3O[C@@H](c3onc5c3CNCC5)C4)c2s1]

### (S)-1-(4-((R)-5-chloro-2-(4,5,6,7-tetrahydroisoxazolo[4,3-c]pyridin-3-yl)-2,3-dihydrobenzofuran-7-yl)thieno[3,2-d]pyrimidin-6-yl)propan-1-ol

The title compound was synthesized using precursor III, general procedure G and D using 1-(4-chlorothieno[3,2-d]pyrimidin-6-yl)propan-1-ol to give the desired compound as the HCl salt. 1H NMR (400 MHz, CD3OD): δ ppm 9.09 (s, 1H), 7.63 (s, 1H), 7.55 (s, 1H), 7.39 (s, 1), 6.09 (bs, 1H), 4.98 (q, J=6.9 Hz, 1H), 4.45-4.21 (m, 2H), 3.96-3.55 (m, 4H), 2.79 (t, J=5.2 Hz, 2H), 1.98-1.89 (m, 2H), 0.98 (t, J=6.2 Hz, 3H). [M+H] 469.1.

### Example 193

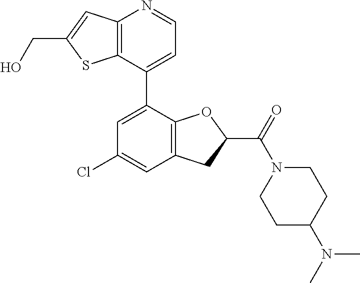 [SMILES: CN(C)C1CCN(C(=O)[C@H]2Cc3cc(Cl)cc(-c4ccnc5cc(CO)sc45)c3O2)CC1]

### (R)-(5-chloro-7-(2-(hydroxymethyl)thieno[3,2-b]pyridin-7-yl)-2,3-dihydrobenzofuran-2-yl)(4-(dimethylamino)piperidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.68 (d, J=4.8 Hz, 1H), 7.48 (s, 1H), 7.32 (dt, J=2.4, 1.1 Hz, 1H), 7.23 (d, J=2.2 Hz, 1H), 7.13 (d, J=4.8 Hz, 1H), 5.41 (t, J=9.1 Hz, 1H), 4.90-4.80 (m, 2H), 4.69 (d, J=13.8 Hz, 1H), 4.21-3.96 (m, 3H), 3.27 (dd, J=16.3, 9.7 Hz, 1H), 2.68-2.47 (m, 2H), 2.23 (s, 6H), 1.96 (d, J=12.8 Hz, 1H), 1.55 (d, J=25.1 Hz, 1H), 1.36 (qd, J=12.5, 4.4 Hz, 1H), 0.67 (qd, J=12.3, 4.2 Hz, 1H). [M+H] 472.1.

### Example 194

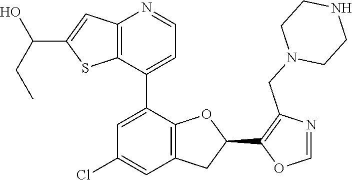 [SMILES: CCC(O)c1cc2nccc(-c3cc(Cl)cc4c3O[C@@H](c3ocnc3CN3CCNCC3)C4)c2s1]

### 1-(7-((R)-5-chloro-2-(4-(piperazin-1-ylmethyl)oxazol-5-yl)-2,3-dihydrobenzofuran-7-yl)thieno[3,2-b]pyridin-2-yl)propan-1-ol

The title compound was synthesized using precursor VII, general procedure C, E and F using 1-(7-(4,4,5,5-tetramethyl-1,3,2-dioxaborolan-2-yl)thieno[3,2-b]pyridin-2-yl)propan-1-ol to give the desired product. 1H NMR (400 MHz, Methanol-d) δ 8.58 (dd, J=5.0, 1.7 Hz, 1H), 8.18-8.15 (m, 1H), 7.50-7.43 (m, 2H), 7.39-7.36 (m, 2H), 6.22 (qd, J=7.3, 3.4 Hz, 1H), 3.93 (s, 1H), 3.68 (dd, J=18.4, 8.5 Hz, 1H), 3.56-3.49 (m, 2H), 2.67 (t, J=5.1 Hz, 2H), 2.53 (q, J=4.8 Hz, 1H), 2.40 (q, J=15.5, 12.0 Hz, 3H), 1.92-1.78 (m, 1H), 0.99 (q, J=7.5 Hz, 3H). [M+H] 551.0.

### Example 195

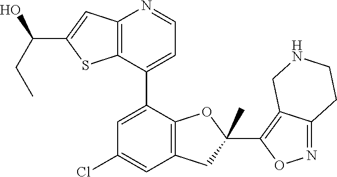 [SMILES: CC[C@@H](O)c1cc2nccc(-c3cc(Cl)cc4c3O[C@@](C)(c3onc5c3CNCC5)C4)c2s1]

### (R)-1-(7-((S)-5-chloro-2-methyl-2-(4,5,6,7-tetrahydroisoxazolo[4,3-c]pyridin-3-yl)-2,3-dihydrobenzofuran-7-yl)thieno[3,2-b]pyridin-2-yl)propan-1-ol

Isomer of title compound from Example 156 obtained after chromatography. 1H NMR (400 MHz, Methanol-d) δ 8.64 (d, J=5.0 Hz, 1H), 7.50 (d, J=2.2 Hz, 1H), 7.45 (d, J=4.9 Hz, 1H), 7.42-7.39 (m, 2H), 4.95 (t, J=6.5 Hz, 1H), 3.78-3.68 (m, 1H), 3.54 (d, J=16.3 Hz, 1H), 3.55-3.39 (m, 2H), 2.89 (t, J=6.0 Hz, 2H), 2.70 (t, J=5.9 Hz, 2H), 1.93-1.84 (m, 5H), 1.01 (t, J=7.4 Hz, 3H).

### Example 196

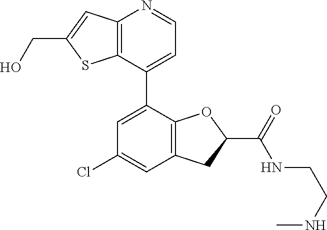 [SMILES: CNCCNC(=O)[C@H]1Cc2cc(Cl)cc(-c3ccnc4cc(CO)sc34)c2O1]

### (R)-5-chloro-7-(2-(hydroxymethyl)thieno[3,2-b]pyridin-7-yl)-N-(2-(methylamino)ethyl)-2,3-dihydrobenzofuran-2-carboxamide

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, CD3OD) δ 8.89 (d, J=6.1 Hz, 1H), 8.43 (dd, J=8.4, 1.4 Hz, 1H), 8.11 (d, J=6.1 Hz, 1H), 7.77-7.65 (m, 1H), 7.56-7.48 (m, 1H), 5.45 (dd, J=10.6, 7.5 Hz, 1H), 5.07 (d, J=1.2 Hz, 2H), 3.65 (s, 4H), 3.61-3.46 (m, 2H), 3.12 (t, J=5.9 Hz, 1H), 2.68 (s, 2H). [M+H] 418.1.

### Example 197

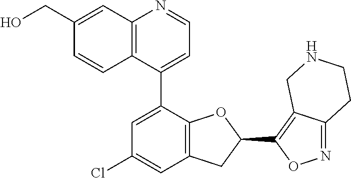 [SMILES: OCc1ccc2c(-c3cc(Cl)cc4c3O[C@@H](c3onc5c3CNCC5)C4)ccnc2c1]

### (R)-(4-(5-chloro-2-(4,5,6,7-tetrahydroisoxazolo[4,3-c]pyridin-3-yl)-2,3-dihydrobenzofuran-7-yl)quinolin-7-yl)methanol

The title compound was synthesized using precursor III, general procedure C and D using (4-chloroquinolin-7-yl)methanol to give the desired product as the HCl salt. 1H NMR (400 MHz, CD3OD): δ ppm 9.22 (d, J=5.4 Hz, 1H), 8.30 (s, 1H), 8.17-7.96 (m, 2H), 7.85 (d, J=8.6 Hz, 1H), 7.61 (s, 1H), 7.48 (d, J=1.8 Hz, 1H), 6.26-5.91 (m, 1H), 4.95 (s, 2H), 4.06 (s, 1H), 3.91 (dd, J=16.2, 10.1 Hz, 1H), 3.75-3.60 (m, 2H), 3.45 (d, J=6.0 Hz, 2H), 3.09 (t, J=5.9 Hz, 2H). [M+H] 434.0.

### Example 198

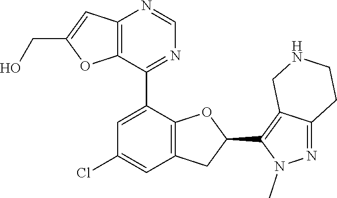 [SMILES: Cn1nc2c(c1[C@H]1Cc3cc(Cl)cc(-c4ncnc5cc(CO)oc45)c3O1)CNCC2]

### (R)-(4-(5-chloro-2-(2-methyl-4,5,6,7-tetrahydro-2H-pyrazolo[4,3-c]pyridin-3-yl)-2,3-dihydrobenzofuran-7-yl)furo[3,2-d]pyrimidin-6-yl)methanol

The title compound was synthesized using precursor IV, general procedure G and D using (4-chlorofuro[3,2-d]pyrimidin-6-yl)methanol to give the desired product as the HCl salt. 1H NMR (400 MHz, CD3OD) δ 9.05 (s, 1H), 7.74 (d, J=2.2 Hz, 1H), 7.49-7.39 (m, 1H), 7.03 (s, 1H), 5.94 (dd, J=9.7, 7.5 Hz, 1H), 4.72-4.64 (m, 2H), 4.19-3.97 (m, 2H), 3.64 (s, 5H), 3.53 (s, 2H), 3.48-3.29 (m, 2H), 2.89 (t, J=6.2 Hz, 2H). [M+H] 438.1.

### Example 199

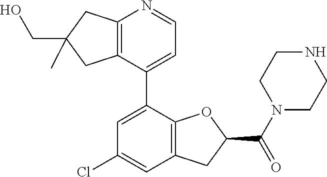 [SMILES: CC1(CO)Cc2nccc(-c3cc(Cl)cc4c3O[C@@H](C(=O)N3CCNCC3)C4)c2C1]

### ((2R)-5-chloro-7-(6-(hydroxymethyl)-6-methyl-6,7-dihydro-5H-cyclopenta[b]pyridin-4-yl)-2,3-dihydrobenzofuran-2-yl)(piperazin-1-yl)methanone

The title compound was synthesized using precursor II, general procedure B, E and F using (4-chloro-6-methyl-6,7-dihydro-5H-cyclopenta[b]pyridin-6-yl)methanol to give the desired product. 1H NMR (400 MHz, Methanol-d4) δ 8.51-8.43 (m, 1H), 7.86-7.79 (m, 1H), 7.45-7.38 (m, 1H), 7.36-7.30 (m, 1H), 5.87-5.78 (m, 1H), 3.87 (s, 4H), 3.62 (d, J=8.5 Hz, 2H), 3.53-3.42 (m, 2H), 3.39-2.65 (m, 8H), 1.19 (s, 3H). [M+H] 428.0.

### Example 200

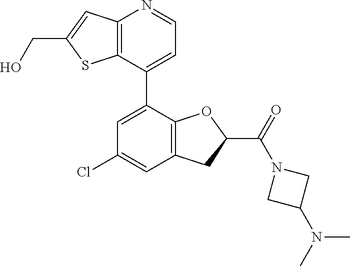 [SMILES: CN(C)C1CN(C(=O)[C@H]2Cc3cc(Cl)cc(-c4ccnc5cc(CO)sc45)c3O2)C1]

### (R)-(5-chloro-7-(2-(hydroxymethyl)thieno[3,2-b]pyridin-7-yl)-2,3-dihydrobenzofuran-2-yl)(3-(dimethylamino)azetidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.70 (dd, J=4.9, 3.1 Hz, 1H), 7.49-7.19 (m, 4H), 5.32 (ddd, J=79.8, 10.3, 7.2 Hz, 1H), 5.05-4.82 (m, 2H), 4.55-3.26 (m, 7H), 2.16 (s, 4H). [M+H] 444.0.

### Example 201

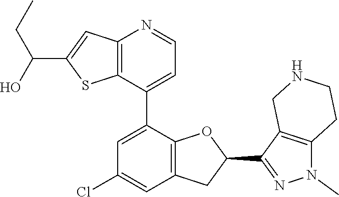 [SMILES: CCC(O)c1cc2nccc(-c3cc(Cl)cc4c3O[C@@H](c3nn(C)c5c3CNCC5)C4)c2s1]

### 1-(7-((R)-5-chloro-2-(1-methyl-4,5,6,7-tetrahydro-1H-pyrazolo[4,3-c]pyridin-3-yl)-2,3-dihydrobenzofuran-7-yl)thieno[3,2-b]pyridin-2-yl)propan-1-ol

The title compound was synthesized using precursor II, general procedure C and D using 1-(7-chlorothieno[3,2-b]pyridin-2-yl)propan-1-ol to give the desired product as the HCl salt. 1H NMR (400 MHz, CD3OD): δ ppm 8.89 (d, J=6.1 Hz, 1H), 8.01 (d, J=6.0 Hz, 1H), 7.70 (d, J=2.4 Hz, 1H), 7.66 (dd, J=2.6, 1.0 Hz, 1H), 7.64 (s, 1H), 6.28 (t, J=9.0 Hz, 1H), 5.17-5.05 (m, 1H), 4.28-4.08 (m, 1H), 4.03 (dd, J=14.6, 4.3 Hz, 1H), 3.92 (dd, J=16.8, 9.9 Hz, 1H), 3.85 (s, 3H), 3.55-3.33 (m, 3H), 2.96 (d, J=3.2 Hz, 2H), 2.10-1.75 (m, 2H), 1.04 (q, J=7.3 Hz, 3H). [M+H] 481.0.

### Example 202

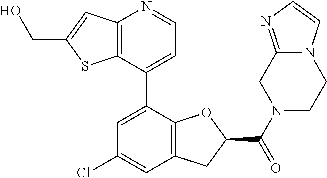 [SMILES: O=C([C@H]1Cc2cc(Cl)cc(-c3ccnc4cc(CO)sc34)c2O1)N1CCn2ccnc2C1]

### (R)-(5-chloro-7-(2-(hydroxymethyl)thieno[3,2-b]pyridin-7-yl)-2,3-dihydrobenzofuran-2-yl)(5,6-dihydroimidazo[1,2-a]pyrazin-7(8H)-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, CD3OD): δ ppm 8.61 (d, J=4.8 Hz, 1H), 7.58-7.45 (4H), 7.05-6.91 (m, 2H), 5.81 (q, J=8.8 Hz, 1H), 4.97-4.63 (m, 4H), 4.07-3.77 (m, 5H), 3.57-3.46 (m, 2H). [M+H] 467.0.

### Example 203

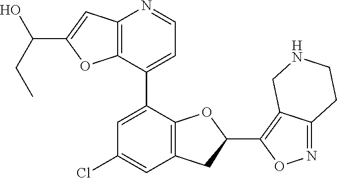 [SMILES: CCC(O)c1cc2nccc(-c3cc(Cl)cc4c3O[C@@H](c3onc5c3CNCC5)C4)c2o1]

### 1-(7-((R)-5-chloro-2-(4,5,6,7-tetrahydroisoxazolo[4,3-c]pyridin-3-yl)-2,3-dihydrobenzofuran-7-yl)furo[3,2-b]pyridin-2-yl)propan-1-ol

The title compound was synthesized using precursor III, general procedure C, E and F using 1-(7-chlorofuro[3,2-b]pyridin-2-yl)propan-1-ol to give the desired product. 1H NMR (400 MHz, Methanol-d) δ 8.46 (d, J=5.3 Hz, 1H), 7.78 (s, 1H), 7.64 (dd, J=5.2, 3.5 Hz, 1H), 7.44 (s, 1H), 7.32 (dd, J=8.3, 4.9 Hz, 1H), 6.14 (dd, J=10.0, 6.2 Hz, 1H), 4.80-4.68 (m, 1H), 3.82 (dd, J=16.8, 10.3 Hz, 1H), 3.69 (qd, J=15.6, 2.0 Hz, 2H), 3.53 (dd, J=16.5, 6.1 Hz, 1H), 3.05-2.97 (m, 2H), 2.78 (t, J=6.0 Hz, 2H), 2.07-1.80 (m, 2H), 1.05-0.96 (m, 3H). [M+H] 452.0.

### Example 204

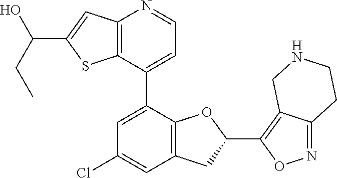 [SMILES: CCC(O)c1cc2nccc(-c3cc(Cl)cc4c3O[C@H](c3onc5c3CNCC5)C4)c2s1]

### 1-(7-((S)-5-chloro-2-(4,5,6,7-tetrahydroisoxazolo[4,3-c]pyridin-3-yl)-2,3-dihydrobenzofuran-7-yl)thieno[3,2-b]pyridin-2-yl)propan-1-ol

The title compound was synthesized using precursor III, general procedure C, E and F using 1-(7-chlorothieno[3,2-b]pyridin-2-yl)propan-1-ol to give the desired product. 1H NMR (400 MHz, Methanol-d) δ 8.61 (d, J=5.0 Hz, 1H), 7.52-7.50 (m, 1H), 7.47-7.45 (m, 1H), 7.43 (dd, J=5.0, 1.6 Hz, 1H), 7.40-7.38 (m, 1H), 6.09 (dd, J=10.0, 6.1 Hz, 1H), 4.97-4.90 (m, 1H), 3.81 (dd, J=16.5, 10.0 Hz, 1H), 3.59 (dd, J=13.2, 8.5 Hz, 2H), 3.61-3.50 (m, 1H), 2.96 (t, J=6.1 Hz, 2H), 2.74 (t, J=6.0 Hz, 2H), 1.93-1.82 (m, 2H), 1.01 (t, J=7.4 Hz, 3H). [M+H] 468.0.

### Example 205

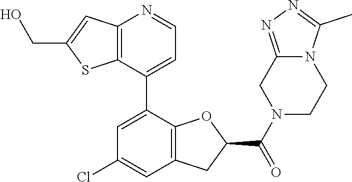 [SMILES: Cc1nnc2n1CCN(C(=O)[C@H]1Cc3cc(Cl)cc(-c4ccnc5cc(CO)sc45)c3O1)C2]

### (R)-(5-chloro-7-(2-(hydroxymethyl)thieno[3,2-b]pyridin-7-yl)-2,3-dihydrobenzofuran-2-yl)(3-methyl-5,6-dihydro-[1,2,4]triazolo[4,3-a]pyrazin-7(8H)-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, CD3OD) δ 8.65 (d, J=4.9 Hz, 1H), 7.46 (d, J=4.9 Hz, 1H), 7.45-7.38 (m, 4H), 5.86 (ddd, J=15.7, 9.9, 6.7 Hz, 1H), 4.86-4.81 (m, 2H), 4.48-4.34 (m, 1H), 4.28-4.20 (m, 1H), 4.19-4.04 (m, 2H), 4.03-3.91 (m, 4H), 3.00-2.94 (m, 3H). [M+H] 482.1.

### Example 206

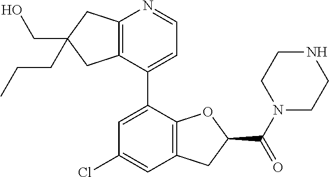 [SMILES: CCCC1(CO)Cc2nccc(-c3cc(Cl)cc4c3O[C@@H](C(=O)N3CCNCC3)C4)c2C1]

### ((2R)-5-chloro-7-(6-(hydroxymethyl)-6-propyl-6,7-dihydro-5H-cyclopenta[b]pyridin-4-yl)-2,3-dihydrobenzofuran-2-yl)(piperazin-1-yl)methanone

The title compound was synthesized using precursor II, general procedure B, E and F using (4-chloro-6-propyl-6,7-dihydro-5H-cyclopenta[b]pyridin-6-yl)methanol to give the desired product. 1H NMR (400 MHz, Methanol-d4) δ 8.47 (s, 1H), 7.77 (s, 1H), 7.41 (s, 1H), 7.33 (s, 1H), 5.82 (dd, J=9.6, 7.0 Hz, 1H), 3.95-3.76 (m, 4H), 3.67-3.55 (m, 2H), 3.55-2.80 (m, 10H), 1.62-1.47 (m, 2H), 1.39-1.17 (m, 2H), 0.93 (t, J=7.2 Hz, 3H). [M+H] 456.1.

### Example 207

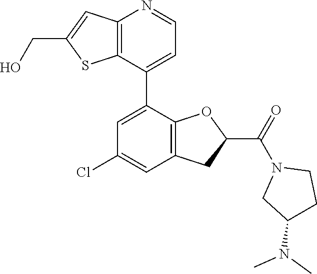 [SMILES: CN(C)[C@H]1CCN(C(=O)[C@H]2Cc3cc(Cl)cc(-c4ccnc5cc(CO)sc45)c3O2)C1]

### ((R)-5-chloro-7-(2-(hydroxymethyl)thieno[3,2-b]pyridin-7-yl)-2,3-dihydrobenzofuran-2-yl)((S)-3-(dimethylamino)pyrrolidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.68 (t, J=5.2 Hz, 1H), 7.42 (d, J=9.4 Hz, 1H), 7.36-7.15 (m, 3H), 5.48-5.34 (m, 1H), 4.95 (d, J=15.9 Hz, 2H), 4.01-3.21 (m, 6H), 2.71 (s, 2H), 2.22 (s, 4H), 2.14-1.98 (m, 1H), 1.61-1.11 (m, 2H). [M+H] 458.0.

### Example 208

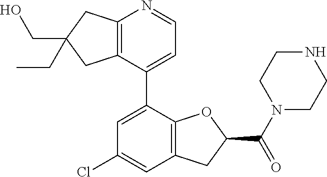 [SMILES: CCC1(CO)Cc2nccc(-c3cc(Cl)cc4c3O[C@@H](C(=O)N3CCNCC3)C4)c2C1]

### ((2R)-5-chloro-7-(6-ethyl-6-(hydroxymethyl)-6,7-dihydro-5H-cyclopenta[b]pyridin-4-yl)-2,3-dihydrobenzofuran-2-yl)(piperazin-1-yl)methanone

The title compound was synthesized using precursor II, general procedure B, E and F using (4-chloro-6-ethyl-6,7-dihydro-5H-cyclopenta[b]pyridin-6-yl)methanol to give the desired product. 1H NMR (400 MHz, Methanol-d4) δ 8.64-8.31 (m, 1H), 7.80-7.62 (m, 1H), 7.44-7.36 (m, 1H), 7.36-7.19 (m, 1H), 5.88-5.72 (m, 1H), 3.88 (s, 4H), 3.67-3.54 (m, 2H), 3.54-2.59 (m, 10H), 1.77-1.47 (m, 2H), 1.01-0.67 (m, 3H). [M+H] 442.0.

### Example 209

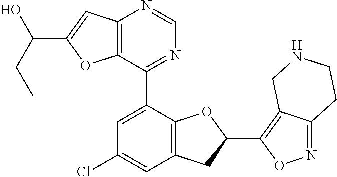 [SMILES: CCC(O)c1cc2ncnc(-c3cc(Cl)cc4c3O[C@@H](c3onc5c3CNCC5)C4)c2o1]

### 1-(4-((R)-5-chloro-2-(4,5,6,7-tetrahydroisoxazolo[4,3-c]pyridin-3-yl)-2,3-dihydrobenzofuran-7-yl)furo[3,2-d]pyrimidin-6-yl)propan-1-ol

The title compound was synthesized using precursor II, general procedure G and C using 1-(4-chlorofuro[3,2-d]pyrimidin-6-yl)propan-1-ol to give the desired product as the HCl salt. 1H NMR (400 MHz, CD3OD): δ ppm 9.04 (s, 1H), 7.84 (s, 1H), 7.73 (s, 1H), 6.97 (d, J=1.8 Hz, 1H), 6.30-6.16 (m, 1H), 4.73-4.50 (m, 3H), 4.36-4.10 (m, 2H), 3.94-3.81 (m, 2H), 3.48 (s, 2H), 1.01 (dd, J=8.9, 5.9 Hz, 3H).

### Example 210

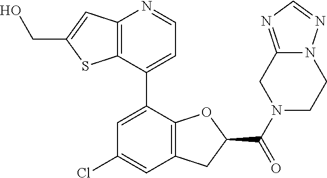 [SMILES: O=C([C@H]1Cc2cc(Cl)cc(-c3ccnc4cc(CO)sc34)c2O1)N1CCn2ncnc2C1]

### (R)-(5-chloro-7-(2-(hydroxymethyl)thieno[3,2-b]pyridin-7-yl)-2,3-dihydrobenzofuran-2-yl)(5,6-dihydro-[1,2,4]triazolo[1,5-a]pyrazin-7(8H)-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, CD3OD) δ 8.65-8.51 (m, 1H), 7.99-7.85 (m, 2H), 7.48-7.32 (m, 3H), 5.84 (ddd, J=20.4, 9.9, 6.7 Hz, 1H), 4.91 (s, 2H), 4.13 (s, 3H), 4.13-3.92 (m, 1H), 3.81-3.67 (m, 1H), 3.58-3.41 (m, 1H), 3.30 (p, J=1.7 Hz, 2H). [M+H] 468.1.

### Example 211

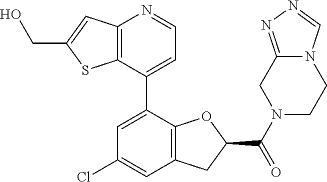 [SMILES: O=C([C@H]1Cc2cc(Cl)cc(-c3ccnc4cc(CO)sc34)c2O1)N1CCn2cnnc2C1]

### (R)-(5-chloro-7-(2-(hydroxymethyl)thieno[3,2-b]pyridin-7-yl)-2,3-dihydrobenzofuran-2-yl)(5,6-dihydro-[1,2,4]triazolo[4,3-a]pyrazin-7(8H)-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, CD3OD) δ 8.63 (dd, J=4.6, 1.4 Hz, 1H), 7.47-7.43 (m, 1H), 7.41 (d, J=1.8 Hz, 4H), 7.39 (d, J=4.7 Hz, 1H), 5.84 (dd, J=9.9, 6.6 Hz, 1H), 4.85-4.72 (m, 3H), 4.15 (t, J=5.1 Hz, 1H), 4.11-4.04 (m, 1H), 3.97-3.78 (m, 2H), 3.78-3.60 (m, 1H), 3.52 (ddt, J=16.4, 10.0, 1.1 Hz, 1H), 3.34 (s, 2H). [M+H] 468.1.

### Example 212

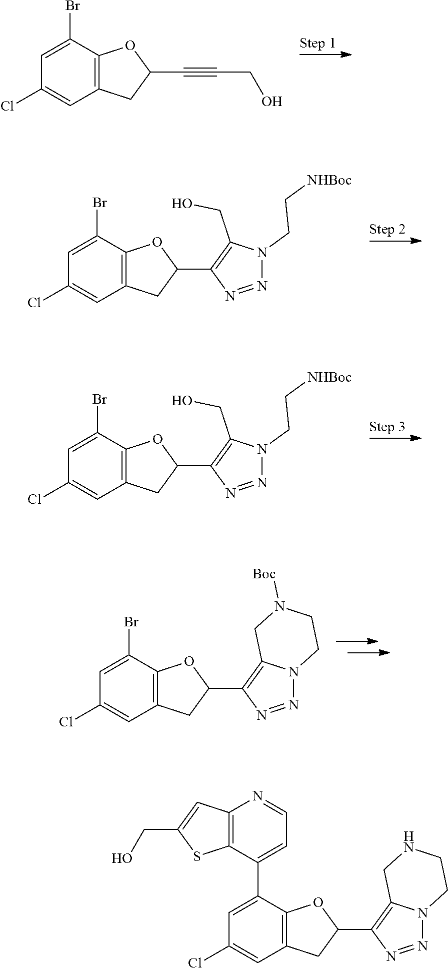 [SMILES: OCC#CC1Cc2cc(Cl)cc(Br)c2O1 ; CC(C)(C)OC(=O)NCCn1nnc(C2Cc3cc(Cl)cc(Br)c3O2)c1CO ; CC(C)(C)OC(=O)NCCn1nnc(C2Cc3cc(Cl)cc(Br)c3O2)c1CO ; CC(C)(C)OC(=O)N1CCn2nnc(C3Cc4cc(Cl)cc(Br)c4O3)c2C1 ; OCc1cc2nccc(-c3cc(Cl)cc4c3OC(c3nnn5c3CNCC5)C4)c2s1]

### (7-(5-chloro-2-(4,5,6,7-tetrahydro-[1,2,3]triazolo[1,5-a]pyrazin-3-yl)-2,3-dihydrobenzofuran-7-yl)thieno[3,2-b]pyridin-2-yl)methanol

### Step 1

Cp*Ru(PPh3)2Cl (69 mg, 0.090 mmol), 2-methyl-2-propanyl-(2-azidoethyl)carbamate (0.32 g, 1.74 mmol) and 3-(7-bromo-5-chloro-2,3-dihydrobenzofuran-2-yl)prop-2-yn-1-ol (0.50 g, 1.74 mmol) in 1,4-dioxane (8 mL) were sparged with Ar and heated to 60° C. in a microwave vial overnight. Concentrated in vacuo, flash chromatography 5-100% EA/hexanes. Obtained two regioisomers: isomer 1=0.32 g (39%), isomer 2=0.27 g (33%).

### Step 2

To tert-butyl N-[2-[4-(7-bromo-5-chloro-2,3-dihydrobenzofuran-2-yl)-5-(hydroxymethyl)triazol-1-yl]ethyl]carbamate (0.32 g, 0.68 mmol) in DCM (3.4 mL) is added tetrabromomethane (0.25 g, 0.75 mmol) and triphenylphosphine (0.20 g, 0.75 mmol).

### Step 3

To sodium hydride (34 mg, 0.86 mmol) in THF (5 mL) is added tert-butyl N-[2-[4-(7-bromo-5-chloro-2,3-dihydrobenzofuran-2-yl)-5-(bromomethyl)triazol-1-yl]ethyl] carbamate (0.31 g, 0.57 mmol) in THF (5 mL) stirred at RT overnight. Diluted with water, ethyl acetate. Extracted 2× with ethyl acetate, dried, filtered, concentrated. Flash chromatography 5-100% EA/hexanes gives 0.21 g (82%).

The title compound was synthesized using general procedure C and D using 7-chloro-2-(((2,3,3-trimethylbutan-2-yl)oxy)methyl)thieno[3,2-b]pyridine to give the desired product as the HCl salt. 1H NMR (400 MHz, Methanol-d4) δ 8.85 (d, J=5.8 Hz, 1H), 7.97 (d, J=5.8 Hz, 1H), 7.69-7.46 (m, 3H), 6.20 (t, J=8.5 Hz, 1H), 5.05 (s, 2H), 4.71 (q, J=5.5 Hz, 2H), 4.64-4.43 (m, 2H), 3.88-3.56 (m, 4H). [M+H] 440.0.

### Example 213

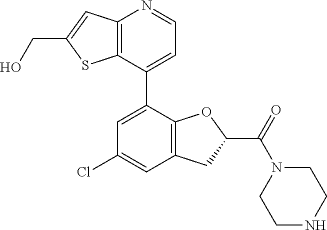 [SMILES: O=C([C@@H]1Cc2cc(Cl)cc(-c3ccnc4cc(CO)sc34)c2O1)N1CCNCC1]

### (S)-(5-chloro-7-(2-(hydroxymethyl)thieno[3,2-b]pyridin-7-yl)-2,3-dihydrobenzofuran-2-yl)(piperazin-1-yl)methanone

The title compound was synthesized using the enantiomer of precursor II, general procedure C and D using 7-chloro-2-(((2,3,3-trimethylbutan-2-yl)oxy)methyl)thieno[3,2-b]pyridine to give the desired product as the HCl salt. 1H NMR (400 MHz, DMSO-d6) δ 9.10 (bs, 2H), 8.74 (d, J=5.0 Hz, 1H), 7.64 (d, J=5.0 Hz, 1H), 7.53 (s, 1H), 7.51-7.47 (m, 2H), 5.82 (t, J=7.8 Hz, 1H), 4.84 (s, 2H), 3.80-3.44 (m, 6H), 3.15-2.88 (m, 4H). [M+H] 430.0.

### Example 214

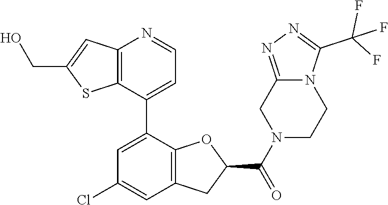 [SMILES: O=C([C@H]1Cc2cc(Cl)cc(-c3ccnc4cc(CO)sc34)c2O1)N1CCn2c(nnc2C(F)(F)F)C1]

### (R)-(5-chloro-7-(2-(hydroxymethyl)thieno[3,2-b]pyridin-7-yl)-2,3-dihydrobenzofuran-2-yl)(3-(trifluoromethyl)-5,6-dihydro-[1,2,4]triazolo[4,3-a]pyrazin-7(8H)-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, CD3OD) δ 8.61 (d, J=5.0 Hz, 1H), 7.54-7.31 (m, 4H), 5.84 (td, J=10.1, 6.7 Hz, 2H), 4.26-4.12 (m, 2H), 3.90 (dd, J=16.2, 6.4 Hz, 1H), 3.72 (p, J=6.7 Hz, 2H), 3.58-3.41 (m, 1H), 3.30 (p, J=1.7 Hz, 2H), 3.22 (q, J=7.4 Hz, 2H). [M+H] 536.1.

### Example 215

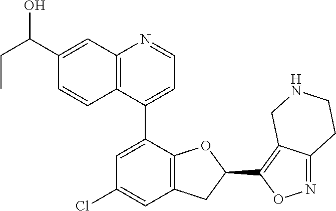 [SMILES: CCC(O)c1ccc2c(-c3cc(Cl)cc4c3O[C@@H](c3onc5c3CNCC5)C4)ccnc2c1]

### 1-(4-((R)-5-chloro-2-(4,5,6,7-tetrahydroisoxazolo[4,3-c]pyridin-3-yl)-2,3-dihydrobenzofuran-7-yl)quinolin-7-yl)propan-1-ol

The title compound was synthesized using precursor III, general procedure C and D using 1-(4-chloroquinolin-7-yl)propan-1-ol to give the desired product as the HCl salt. 1H NMR (400 MHz, CD3OD): δ ppm 9.21 (d, J=5.7 Hz, 1H), 8.26 (d, J=9.3 Hz, 1H), 8.14-7.95 (m, 2H), 7.89 (d, J=9.0 Hz, 1H), 7.62 (s, 1H), 7.48 (d, J=2.1 Hz, 1H), 6.21-6.09 (m, 1H), 4.88-4.92 (m, 1H), 4.08 (s, 1H), 3.91 (dd, J=16.4, 10.0 Hz, 1H), 3.63 (dd, J=22.9, 14.4 Hz, 1H), 3.45 (d, J=6.9 Hz, 2H), 3.09 (t, J=6.1 Hz, 2H), 2.00-1.72 (m, 2H), 1.26 (t, J=7.2 Hz, 1H), 0.99 (td, J=7.4, 3.0 Hz, 3H). [M+H] 462.0.

### Example 216

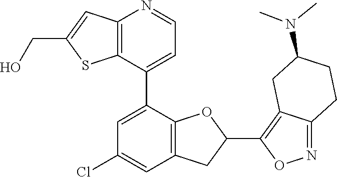 [SMILES: CN(C)[C@H]1CCc2noc(C3Cc4cc(Cl)cc(-c5ccnc6cc(CO)sc56)c4O3)c2C1]

### (7-(5-chloro-2-((S)-5-(dimethylamino)-4,5,6,7-tetrahydrobenzo[c]isoxazol-3-yl)-2,3-dihydrobenzofuran-7-yl)thieno[3,2-b]pyridin-2-yl)methanol

The title compound was synthesized using precursor V, general procedure C and D using (7-chlorothieno[3,2-b]pyridin-2-yl)methanol to give the desired compound. 1H NMR (400 MHz, CD3OD): δ ppm 8.60 (dd, J=5.0, 4.0 Hz, 1H), 7.53 (dq, J=1.8, 0.8 Hz, 1H), 7.50-7.43 (m, 2H), 7.41 (t, J=1.2 Hz, 1H), 6.14-6.04 (m, 1H), 4.89 (dd, J=1.1, 0.4 Hz, 2H), 3.79 (ddq, J=16.3, 9.9, 1.1 Hz, 1H), 3.65-3.50 (m, 1H), 2.96-2.81 (m, 1H), 2.69-2.44 (m, 3H), 2.36-2.15 (m, 1H), 2.11 (s, 3H), 2.07 (s, 3H), 2.06-1.98 (m, 1H), 1.71-1.53 (m, 1H). [M+H] 482.0.

### Example 217

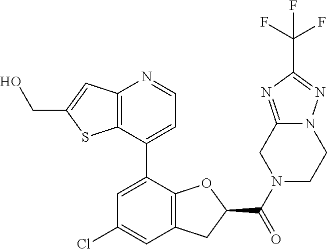 [SMILES: O=C([C@H]1Cc2cc(Cl)cc(-c3ccnc4cc(CO)sc34)c2O1)N1CCn2nc(C(F)(F)F)nc2C1]

### (R)-(5-chloro-7-(2-(hydroxymethyl)thieno[3,2-b]pyridin-7-yl)-2,3-dihydrobenzofuran-2-yl)(2-(trifluoromethyl)-5,6-dihydro-[1,2,4]triazolo[1,5-a]pyrazin-7(8H)-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, CD3OD) δ 8.63 (d, J=5.0 Hz, 1H), 7.49 (d, J=4.9 Hz, 1H), 7.46-7.31 (m, 3H), 5.84 (ddd, J=23.5, 9.9, 6.7 Hz, 1H), 4.25-4.11 (m, 1H), 3.86 (ddd, J=16.5, 8.1, 5.0 Hz, 1H), 3.72 (p, J=6.6 Hz, 2H), 3.30 (p, J=1.7 Hz, 2H), 3.22 (q, J=7.4 Hz, 2H). [M+H] 536.1.

### Example 218

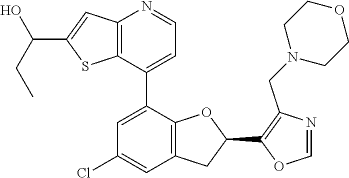 [SMILES: CCC(O)c1cc2nccc(-c3cc(Cl)cc4c3O[C@@H](c3ocnc3CN3CCOCC3)C4)c2s1]

### 1-(7-((R)-5-chloro-2-(4-(morpholinomethyl)oxazol-5-yl)-2,3-dihydrobenzofuran-7-yl)thieno[3,2-b]pyridin-2-yl)propan-1-ol

The title compound was synthesized using precursor VII, general procedure C, E and F using 1-(7-(4,4,5,5-tetramethyl-1,3,2-dioxaborolan-2-yl)thieno[3,2-b]pyridin-2-yl)propan-1-ol to give the desired product. 1H NMR (400 MHz, Methanol-d) δ 8.58 (dd, J=5.0, 1.5 Hz, 1H), 8.17 (d, J=5.5 Hz, 1H), 7.46 (dd, J=8.4, 2.5 Hz, 2H), 7.42-7.33 (m, 2H), 6.22 (ddd, J=9.9, 7.4, 2.4 Hz, 1H), 4.91 (d, J=6.7 Hz, 1H), 3.73 (dd, J=16.4, 9.8 Hz, 1H), 3.63 (dd, J=16.4, 7.6 Hz, 1H), 3.54-3.46 (m, 5H), 2.39-2.32 (m, 6H), 1.89-1.80 (m, 1H), 0.99 (dt, J=9.8, 7.4 Hz, 3H). [M+H] 512.0.

### Example 219

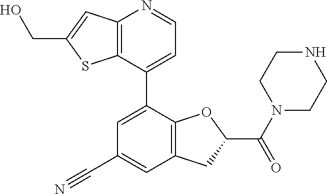 [SMILES: N#Cc1cc2c(c(-c3ccnc4cc(CO)sc34)c1)O[C@H](C(=O)N1CCNCC1)C2]

### (R)-7-(2-(hydroxymethyl)thieno[3,2-b]pyridin-7-yl)-2-(piperazine-1-carbonyl)-2,3-dihydrobenzofuran-5-carbonitrile

The title compound was synthesized using the enantiomer of precursor VIII, general procedure C and D using (7-chlorothieno[3,2-b]pyridin-2-yl)methanol to give the desired compound as the HCl salt. 1H NMR (400 MHz, CD3OD): δ ppm 8.63 (d, J=5.2 Hz, 1H), 7.86 (s, 1H), 7.76 (s, 1H), 7.51 (d, J=5.2 Hz, 1H), 7.43 (s, 1H), 5.840 (dd, J=10.0, 6.8 Hz, 1H), 4.92 (s, 2H), 3.82-3.40 (m, 6H), 2.81-2.51 (m, 4H). [M+H] 421.0.

### Example 220

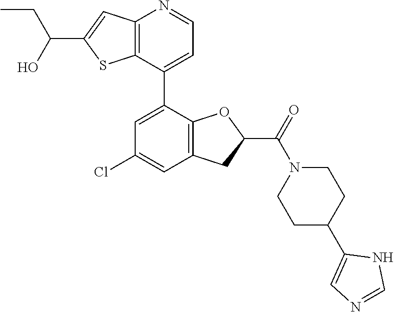 [SMILES: CCC(O)c1cc2nccc(-c3cc(Cl)cc4c3O[C@@H](C(=O)N3CCC(c5cnc[nH]5)CC3)C4)c2s1]

### (4-(1H-imidazol-5-yl)piperidin-1-yl)((2R)-5-chloro-7-(2-(1-hydroxypropyl)thieno[3,2-b]pyridin-7-yl)-2,3-dihydrobenzofuran-2-yl)methanone

The title compound was synthesized using general procedure A and C using 1-(7-(4,4,5,5-tetramethyl-1,3,2-dioxaborolan-2-yl)thieno[3,2-b]pyridin-2-yl)propan-1-ol. The SEM protected imidazole was cleaved with TBAF to give the final product. 1H NMR (400 MHz, Chloroform-d) δ 8.73-8.02 (m, 2H), 7.58-7.15 (m, 5H), 6.12 (d, J=35.8 Hz, 1H), 5.64-5.37 (m, 1H), 5.04 (dt, J=22.7, 6.8 Hz, 1H), 4.73 (s, 2H), 4.60-3.83 (m, 3H), 3.23-2.82 (m, 2H), 2.64 (d, J=13.7 Hz, 2H), 1.90 (td, J=18.8, 16.2, 6.5 Hz, 3H), 1.74-1.55 (m, 3H). [M+H] 523.0.

### Example 221

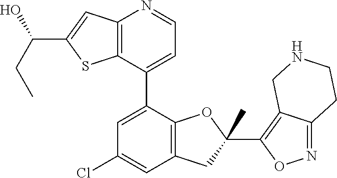 [SMILES: CC[C@H](O)c1cc2nccc(-c3cc(Cl)cc4c3O[C@@](C)(c3onc5c3CNCC5)C4)c2s1]

### (S)-1-(7-((S)-5-chloro-2-methyl-2-(4,5,6,7-tetrahydroisoxazolo[4,3-c]pyridin-3-yl)-2,3-dihydrobenzofuran-7-yl)thieno[3,2-b]pyridin-2-yl)propan-1-ol

Isomer of title compound from Example 156 obtained after chromatography. 1H NMR (400 MHz, Methanol-d) δ 8.63 (d, J=5.0 Hz, 1H), 7.51 (d, J=2.2 Hz, 1H), 7.46 (d, J=5.0 Hz, 1H), 7.44-7.38 (m, 2H), 4.96-4.91 (m, 1H), 3.72 (d, J=16.4 Hz, 1H), 3.59-3.43 (m, 3H), 2.89 (t, J=6.0 Hz, 2H), 2.70 (t, J=6.0 Hz, 2H), 1.95-1.85 (m, 5H), 1.02 (t, J=7.4 Hz, 3H).

### Example 222

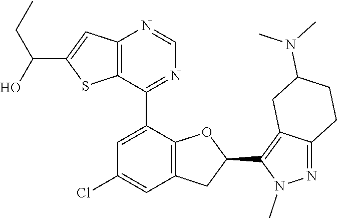 [SMILES: CCC(O)c1cc2ncnc(-c3cc(Cl)cc4c3O[C@@H](c3c5c(nn3C)CCC(N(C)C)C5)C4)c2s1]

### 1-(4-((2R)-5-chloro-2-(5-(dimethylamino)-2-methyl-4,5,6,7-tetrahydro-2H-indazol-3-yl)-2,3-dihydrobenzofuran-7-yl)thieno[3,2-d]pyrimidin-6-yl)propan-1-ol

The product of Step 4, Precursor V was subjected to general procedure C using 7-(4,4,5,5-tetramethyl-1,3,2-dioxaborolan-2-yl)-2-(((2,3,3-trimethylbutan-2-yl)oxy)methyl)thieno[3,2-b]pyridine. The title compound was synthesized by reductive amination using dimethylamine and sodium triacetoxyborohydride to give the desired product. 1H NMR (400 MHz, CD3OD) δ 8.94 (d, J=1.6 Hz, 1H), 7.42 (td, J=3.6, 2.2 Hz, 1H), 7.33 (tt, J=2.1, 1.2 Hz, 1H), 7.27-7.14 (m, 1H), 5.81 (dddd, J=12.1, 9.6, 7.5, 2.2 Hz, 1H), 4.89-4.79 (m, 1H), 3.18 (p, J=1.6 Hz, 2H), 2.76-2.55 (m, 1H), 2.55-2.32 (m, 3H), 2.30-2.10 (m, 1H), 2.04 (d, J=12.8 Hz, 12H), 1.98-1.87 (m, 1H), 1.87-1.61 (m, 2H), 1.61-1.44 (m, 1H). [M+H] 524.1.

### Example 223

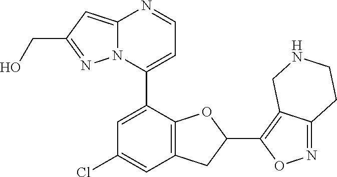 [SMILES: OCc1cc2nccc(-c3cc(Cl)cc4c3OC(c3onc5c3CNCC5)C4)n2n1]

### (7-(5-chloro-2-(4,5,6,7-tetrahydroisoxazolo[4,3-c]pyridin-3-yl)-2,3-dihydrobenzofuran-7-yl)pyrazolo[1,5-a]pyrimidin-2-yl)methanol

The title compound was synthesized using precursor III, general procedure G and D using 2-bromo-7-chloropyrazolo[1,5-a]pyrimidine to give the desired product as the HCl salt after vinyl Stille coupling and oxidation/reduction of the bromide intermediate. 1H NMR (400 MHz, CD3OD): δ ppm 8.61 (bs, 1H), 7.76 (s, 1H), 7.54 (s, 1H), 7.15 (s, 1H), 6.79 (s, 1H), 6.15 (dd, J=9.6, 6.4 Hz, 1H), 4.13-4.01 (m, 2H), 3.86-3.51 (m, 5H), 3.08 (t, J=6.4 Hz, 2H). [M+H] 424.0.

### Example 224

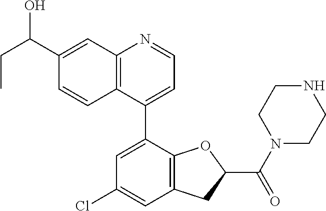 [SMILES: CCC(O)c1ccc2c(-c3cc(Cl)cc4c3O[C@@H](C(=O)N3CCNCC3)C4)ccnc2c1]

### ((2R)-5-chloro-7-(7-(1-hydroxypropyl)quinolin-4-yl)-2,3-dihydrobenzofuran-2-yl)(piperazin-1-yl)methanone

The title compound was synthesized using precursor II, general procedure B and D using 1-(4-chloroquinolin-7-yl)propan-1-ol to give the desired product as the HCl salt. 1H NMR (400 MHz, CD3OD): δ ppm 9.18 (s, 1H), 8.27 (d, J=8.8 Hz, 1H), 8.23 (d, J=7.6 Hz, 1H), 8.10 (d, J=4.8 Hz, 1H), 7.93 (dd, J=8.8, 7.2 Hz, 1H), 7.64 (m, 1H), 7.57 (s, 1H), 7.54 (s, 1H), 5.81 (dt, J=6.4, 2.8 Hz, 1H), 4.98-4.92 (m, 1H), 3.80-3.13 (m, 11H), 2.00-1.86 (m, 2H), 0.95 (t, J=7.2 Hz, 3H). [M+H] 452.0.

### Example 225

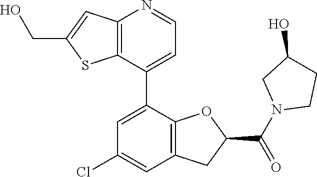 [SMILES: O=C([C@H]1Cc2cc(Cl)cc(-c3ccnc4cc(CO)sc34)c2O1)N1CC[C@H](O)C1]

### ((R)-5-chloro-7-(2-(hydroxymethyl)thieno[3,2-b]pyridin-7-yl)-2,3-dihydrobenzofuran-2-yl)((S)-3-hydroxypyrrolidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, CD3OD) δ 8.59 (dd, J=5.0, 1.1 Hz, 1H), 7.54-7.49 (m, 1H), 7.49-7.44 (m, 1H), 7.42-7.40 (m, 1H), 7.36 (dq, J=2.3, 1.2 Hz, 1H), 5.66-5.51 (m, 1H), 4.40 (dddt, J=10.3, 6.3, 4.4, 2.1 Hz, 1H), 3.76-3.63 (m, 1H), 3.63-3.52 (m, 2H), 3.52-3.44 (m, 1H), 3.30 (p, J=1.7 Hz, 2H), 2.11-1.83 (m, 2H). [M+H] 431.1.

### Example 226

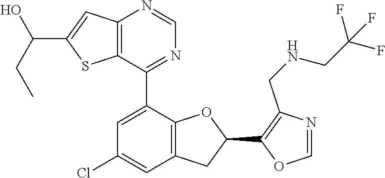 [SMILES: CCC(O)c1cc2ncnc(-c3cc(Cl)cc4c3O[C@@H](c3ocnc3CNCC(F)(F)F)C4)c2s1]

### 1-(4-((R)-5-chloro-2-(4-(((2,2,2-trifluoroethyl)amino)methyl)oxazol-5-yl)-2,3-dihydrobenzofuran-7-yl)thieno[3,2-d]pyrimidin-6-yl)propan-1-ol

The title compound was synthesized using precursor VII, general procedure C, E and F using 1-(7-(4,4,5,5-tetramethyl-1,3,2-dioxaborolan-2-yl)thieno[3,2-b]pyridin-2-yl)propan-1-ol to give the desired product.

### Example 227

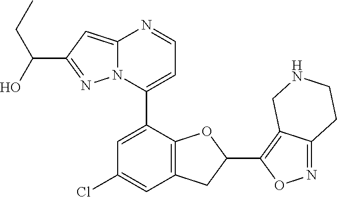 [SMILES: CCC(O)c1cc2nccc(-c3cc(Cl)cc4c3OC(c3onc5c3CNCC5)C4)n2n1]

### 1-(7-(5-chloro-2-(4,5,6,7-tetrahydroisoxazolo[4,3-c]pyridin-3-yl)-2,3-dihydrobenzofuran-7-yl)pyrazolo[1,5-a]pyrimidin-2-yl)propan-1-ol

The title compound was synthesized using precursor III, general procedure G and D using 2-bromo-7-chloropyrazolo[1,5-a]pyrimidine to give the desired product as the HCl salt after vinyl Stille coupling and oxidation/Grignard addition of the bromide intermediate. 1H NMR (400 MHz, CD3OD, hydrochloric salt): δ ppm 8.53 (bs, 1H), 7.82 (s, 1H), 7.55 (s, 1H), 7.15 (d, J=4.4 Hz, 1H), 6.76 (s, 1H), 6.16 (q, J=7.5 Hz, 1H), 4.75-4.69 (m 1H), 4.18-4.00 (m, 2H), 3.90-3.53 (m, 5H), 3.09 (t, J=9.2 Hz, 2H), 1.93 (quin, J=7.5 Hz, 2h), 0.98 (t, J=7.5 Hz, 3H). [M+H] 452.0.

### Example 228

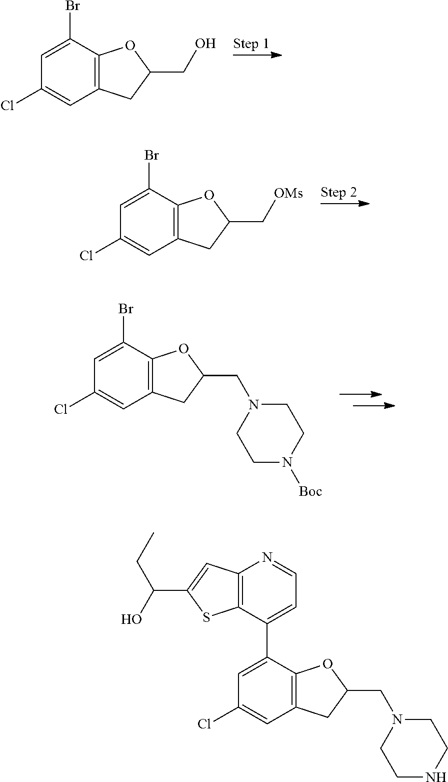 [SMILES: OCC1Cc2cc(Cl)cc(Br)c2O1 ; CS(=O)(=O)OCC1Cc2cc(Cl)cc(Br)c2O1 ; CC(C)(C)OC(=O)N1CCN(CC2Cc3cc(Cl)cc(Br)c3O2)CC1 ; CCC(O)c1cc2nccc(-c3cc(Cl)cc4c3OC(CN3CCNCC3)C4)c2s1]

### 1-(7-(5-chloro-2-(piperazin-1-ylmethyl)-2,3-dihydrobenzofuran-7-yl)thieno[3,2-b]pyridin-2-yl)propan-1-ol

### Step 1

To a solution of (7-bromo-5-chloro-2,3-dihydrobenzofuran-2-yl)methanol (1.25 g, 4.74 mmol) and triethylamine (0.99 mL, 7.12 mmol) in DCM (15.8 mL) at 0° C. was added methanesulfonyl chloride (0.55 mL, 7.12 mmol), immediately warmed to rt. After 1 hr reaction quenched with water, extracted with EtOAc, dried Na₂SO₄, concentrated.

### Step 2

To the crude mesylate was added 15 mL of CH₃CN then Boc-piperazine (1.32 g, 7.12 mmol) and potassium carbonate (1.3 g, 9.49 mmol) and the solution was refluxed overnight. Cooled, filtered, evaporated and loaded directly onto silica column 0-100% EtOAc in hexanes to give the product.

The title compound was synthesized using general procedure C and D using 1-(7-(4,4,5,5-tetramethyl-1,3,2-dioxaborolan-2-yl)thieno[3,2-b]pyridin-2-yl)propan-1-ol to give the desired product as the HCl salt. 1H NMR (400 MHz, CD3OD): δ ppm 8.88 (d, J=5.9 Hz, 1H), 8.13-8.08 (m, 1H), 7.69-7.64 (m, 2H), 7.55-7.54 (m, 1H), 5.58-5.45 (m, 1H), 5.13-5.06 (m, 1H), 3.62-3.47 (m, 11H), 3.23-3.15 (m, 1H), 2.03-1.84 (m, 2H), 1.10-1.01 (m, 3H). [M+H] 444.0.

### Example 229

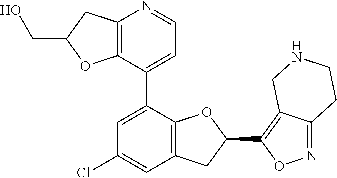 [SMILES: OCC1Cc2nccc(-c3cc(Cl)cc4c3O[C@@H](c3onc5c3CNCC5)C4)c2O1]

### (7-((R)-5-chloro-2-(4,5,6,7-tetrahydroisoxazolo[4,3-c]pyridin-3-yl)-2,3-dihydrobenzofuran-7-yl)-2,3-dihydrofuro[3,2-b]pyridin-2-yl)methanol

The title compound was synthesized using precursor III, general procedure G and D using (7-chloro-2,3-dihydrofuro[3,2-b]pyridin-2-yl)methanol to give the desired product as the HCl salt. 1H NMR (400 MHz, Methanol-d4) δ 7.85-7.79 (m, 2H), 7.28 (dt, J=2.2, 1.1 Hz, 1H), 7.16 (dd, J=8.5, 1.8 Hz, 1H), 6.22 (dd, J=10.0, 5.4 Hz, 1H), 5.07-4.95 (m, 1H), 4.19 (dd, J=15.3, 2.9 Hz, 1H), 4.00 (dd, J=15.3, 1.0 Hz, 1H), 3.93-3.69 (m, 3H), 3.54-3.36 (m, 4H), 3.29-3.18 (m, 1H), 3.11-3.01 (m, 2H). [M+H] 426.0.

### Example 230

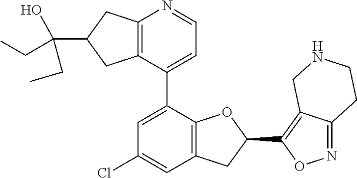 [SMILES: CCC(O)(CC)C1Cc2nccc(-c3cc(Cl)cc4c3O[C@@H](c3onc5c3CNCC5)C4)c2C1]

### 3-(4-((R)-5-chloro-2-(4,5,6,7-tetrahydroisoxazolo[4,3-c]pyridin-3-yl)-2,3-dihydrobenzofuran-7-yl)-6,7-dihydro-5H-cyclopenta[b]pyridin-6-yl)pentan-3-ol

The title compound was synthesized using precursor III, general procedure G, E and F using 3-(4-chloro-6,7-dihydro-5H-cyclopenta[b]pyridin-6-yl)pentan-3-ol to give the desired product. 1H NMR (400 MHz, Methanol-d4) δ 8.50-8.42 (m, 1H), 7.73-7.60 (m, 1H), 7.56-7.45 (m, 1H), 7.43-7.33 (m, 1H), 6.30-6.10 (m, 1H), 4.27 (s, 1H), 4.21-4.04 (m, 1H), 3.98-3.74 (m, 1H), 3.68-3.41 (m, 3H), 3.23-2.94 (m, 3H), 2.94-2.69 (m, 2H), 1.80-1.25 (m, 4H), 1.03-0.77 (m, 6H). [M+H] 480.1.

### Example 231

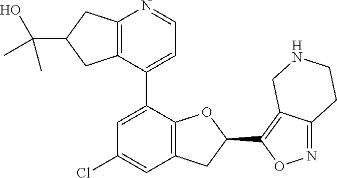 [SMILES: CC(C)(O)C1Cc2nccc(-c3cc(Cl)cc4c3O[C@@H](c3onc5c3CNCC5)C4)c2C1]

### 2-(4-((R)-5-chloro-2-(4,5,6,7-tetrahydroisoxazolo[4,3-c]pyridin-3-yl)-2,3-dihydrobenzofuran-7-yl)-6,7-dihydro-5H-cyclopenta[b]pyridin-6-yl)propan-2-ol

The title compound was synthesized using precursor III, general procedure G, E and F using 2-(4-chloro-6,7-dihydro-5H-cyclopenta[b]pyridin-6-yl)propan-2-ol to give the desired product. 1H NMR (400 MHz, Methanol-d4) δ 8.55-8.44 (m, 1H), 7.80-7.68 (m, 1H), 7.55-7.50 (m, 1H), 7.44-7.38 (m, 1H), 6.30-6.11 (m, 1H), 4.27 (s, 1H), 4.23-4.05 (m, 1H), 3.95-3.75 (m, 1H), 3.67-3.42 (m, 3H), 3.28-3.19 (m, 2H), 3.19-3.07 (m, 2H), 3.07-2.91 (m, 2H), 2.82-2.63 (m, 1H), 1.33-1.09 (m, 6H). [M+H] 452.1.

### Example 232

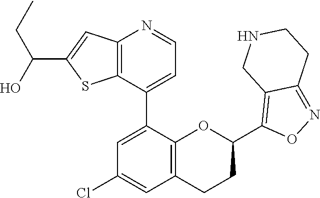 [SMILES: CCC(O)c1cc2nccc(-c3cc(Cl)cc4c3O[C@@H](c3onc5c3CNCC5)CC4)c2s1]

### 1-(7-((R)-6-chloro-2-(4,5,6,7-tetrahydroisoxazolo[4,3-c]pyridin-3-yl)chroman-8-yl)thieno[3,2-b]pyridin-2-yl)propan-1-ol

The title compound was synthesized using precursor X, general procedure C and D using 2-(((tert-butyldimethylsilyl)oxy)methyl)-7-(4,4,5,5-tetramethyl-1,3,2-dioxaborolan-2-yl)thieno[3,2-b]pyridine to give the desired product as the HCl salt. 1H NMR (400 MHz, CD3OD): δ ppm 8.89 (d, J=5.4 Hz, 1H), 7.92 (d, J=5.4 Hz, 1H), 7.64 (s, 1H), 7.48 (s, 1H), 7.47 (s, 1H), 5.59 (d, J=9.6 Hz, 1H), 5.07 (t, J=5.2 Hz, 1H), 4.10-3.87 (m, 2H), 3.65-3.42 (m, 2H), 3.24-2.95 (m, 4H), 2.55-2.36 (m, 2H), 1.99-1.82 (m, 2H), 1.01 (t, J=6.8 Hz, 3H). [M+H] 482.0.

### Example 233

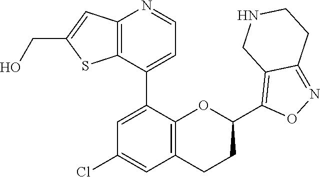 [SMILES: OCc1cc2nccc(-c3cc(Cl)cc4c3O[C@@H](c3onc5c3CNCC5)CC4)c2s1]

### (R)-(7-(6-chloro-2-(4,5,6,7-tetrahydroisoxazolo[4,3-c]pyridin-3-yl)chroman-8-yl)thieno[3,2-b]pyridin-2-yl)methanol

The title compound was synthesized using precursor X, general procedure C and D using 2-(((tert-butyldimethylsilyl)oxy)methyl)-7-(4,4,5,5-tetramethyl-1,3,2-dioxaborolan-2-yl)thieno[3,2-b]pyridine to give the desired product as the HCl salt. 1H NMR (400 MHz, CD3OD): δ ppm 8.90 (d, J=5.2 Hz, 1H), 7.93 (d, J=5.6 Hz, 1H), 7.68 (s, 1H), 7.49 (s, 1H), 7.48 (s, 1H), 5.60 (bd, J=8.8 Hz, 1H), 5.02 (s, 2H), 3.97 (d, J=14.8 Hz, 1H), 3.87 (d, J=14.8 Hz, 1H), 3.44 (bs, 2H), 3.21-2.98 (m, 4H), 2.48-2.28 (m, 2H). [M+H] 454.0.

### Example 234

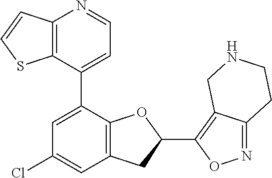 [SMILES: Clc1cc2c(c(-c3ccnc4ccsc34)c1)O[C@@H](c1onc3c1CNCC3)C2]

### (R)-3-(5-chloro-7-(thieno[3,2-b]pyridin-7-yl)-2,3-dihydrobenzofuran-2-yl)-4,5,6,7-tetrahydroisoxazolo[4,3-c]pyridine

The title compound was synthesized using precursor III, general procedure C and E using 7-(4,4,5,5-tetramethyl-1,3,2-dioxaborolan-2-yl)thieno[3,2-b]pyridine to give the desired product as the TFA salt. 1H NMR (400 MHz, Methanol-d4) δ 8.76 (d, J=7.2 Hz, 1H), 8.26-8.05 (m, 1H), 7.72-7.59 (m, 3H), 7.52 (d, J=3.1 Hz, 1H), 6.20 (dd, J=9.9, 6.8 Hz, 1H), 4.14-3.99 (m, 2H), 3.88 (dd, J=16.4, 9.9 Hz, 1H), 3.59 (dd, J=16.4, 6.8 Hz, 1H), 3.45 (t, J=6.3 Hz, 2H), 3.07 (t, J=6.3 Hz, 2H). [M+H] 410.1.

### Example 235

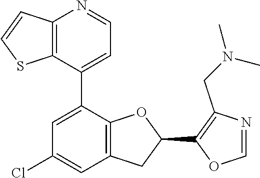 [SMILES: CN(C)Cc1ncoc1[C@H]1Cc2cc(Cl)cc(-c3ccnc4ccsc34)c2O1]

### (R)-1-(5-(5-chloro-7-(thieno[3,2-b]pyridin-7-yl)-2,3-dihydrobenzofuran-2-yl)oxazol-4-yl)-N,N-dimethylmethanamine

The title compound was synthesized using precursor VII, general procedure G using 7-(4,4,5,5-tetramethyl-1,3,2-dioxaborolan-2-yl)thieno[3,2-b]pyridine to give the desired product. 1H NMR (400 MHz, Methanol-d) δ 8.64 (d, J=4.9 Hz, 1H), 8.15 (d, J=0.6 Hz, 1H), 8.01 (dd, J=5.6, 0.4 Hz, 1H), 7.55 (d, J=5.6 Hz, 1H), 7.52-7.49 (m, 1H), 7.48-7.43 (m, 2H), 6.21-6.11 (m, 1H), 3.73 (dd, J=16.4, 9.8 Hz, 1H), 3.61 (dd, J=16.3, 7.6 Hz, 1H), 3.49 (d, J=13.6 Hz, 1H), 3.41 (d, J=13.7 Hz, 1H), 2.16 (s, 6H). [M+H] 412.0.

### Example 236

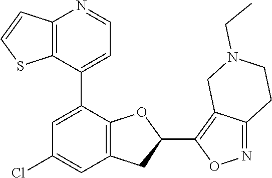 [SMILES: CCN1CCc2noc([C@H]3Cc4cc(Cl)cc(-c5ccnc6ccsc56)c4O3)c2C1]

### (R)-3-(5-chloro-7-(thieno[3,2-b]pyridin-7-yl)-2,3-dihydrobenzofuran-2-yl)-5-ethyl-4,5,6,7-tetrahydroisoxazolo[4,3-c]pyridine

The title compound was synthesized from reductive amination of the title compound from Example 234 using acetaldehyde and sodium triacetoxyborohydride to give the desired product. 1H NMR (400 MHz, Methanol-d4) δ 8.69 (d, J=4.9 Hz, 1H), 8.06 (d, J=5.6 Hz, 1H), 7.62-7.57 (m, 2H), 7.54 (d, J=4.9 Hz, 1H), 7.45 (dd, J=2.2, 1.2 Hz, 1H), 6.15 (dd, J=10.0, 5.3 Hz, 1H), 3.84 (dd, J=16.3, 10.0 Hz, 1H), 3.53 (dd, J=16.3, 5.3 Hz, 1H), 3.22 (d, J=14.5 Hz, 1H), 2.98 (d, J=14.5 Hz, 1H), 2.76 (t, J=5.9 Hz, 2H), 2.68 (dt, J=11.5, 5.6 Hz, 1H), 2.56 (dt, J=12.4, 6.3 Hz, 1H), 2.27 (dt, J=12.4, 7.2 Hz, 1H), 2.15 (dt, J=12.4, 7.2 Hz, 1H), 0.79 (t, J=5.9 Hz, 3H). [M+H] 438.0.

### Example 237

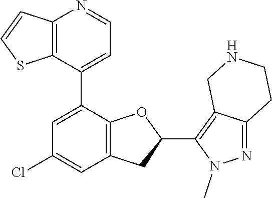 [SMILES: Cn1nc2c(c1[C@H]1Cc3cc(Cl)cc(-c4ccnc5ccsc45)c3O1)CNCC2]

### (R)-7-(5-chloro-2-(2-methyl-4,5,6,7-tetrahydro-2H-pyrazolo[4,3-c]pyridin-3-yl)-2,3-dihydrobenzofuran-7-yl)thieno[3,2-b]pyridine

The title compound was synthesized using precursor IV, general procedure G and D using 7-(4,4,5,5-tetramethyl-1,3,2-dioxaborolan-2-yl)thieno[3,2-b]pyridine to give the desired product as the HCl salt. 1H NMR (400 MHz, Methanol-d4) δ 8.81 (ddd J=5.7, 1.0, 0.9 Hz, 1H), 8.41 (ddd, J=5.7, 1.0, 0.9 Hz, 1H), 7.92-7.81 (m, 1H), 7.73 (ddd, J=5.7, 0.9, 0.9 Hz, 1H), 7.61 (ddd, J=1.7, 1.7, 0.9 Hz, 1H), 7.50 (ddd, J=2.3, 1.2, 1.2 Hz, 1H), 5.99 (ddd, J=9.2, 7.8, 0.9 Hz, 1H), 4.11-3.87 (m, 2H), 3.77-3.57 (m, 6H), 3.46-3.42 (m, 2H), 2.96 (t, J=6.1 Hz, 2H). [M+H] 423.0.

### Example 238

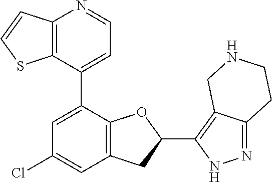 [SMILES: Clc1cc2c(c(-c3ccnc4ccsc34)c1)O[C@@H](c1[nH]nc3c1CNCC3)C2]

### (R)-7-(5-chloro-2-(4,5,6,7-tetrahydro-2H-pyrazolo[4,3-c]pyridin-3-yl)-2,3-dihydrobenzofuran-7-yl)thieno[3,2-b]pyridine

The title compound was synthesized using precursor IV, general procedure G and D using 7-(4,4,5,5-tetramethyl-1,3,2-dioxaborolan-2-yl)thieno[3,2-b]pyridine to give the desired product as the HCl salt. 1H NMR (400 MHz, Methanol-d4) δ 8.76 (d, J=5.2 Hz, 1H), 8.24 (dd, J=5.6, 0.4 Hz, 1H), 7.69 (dd, J=5.3, 0.4 Hz, 1H), 7.66 (d, J=5.6 Hz, 1H), 7.60 (ddd, J=2.2, 2.2, 0.8 Hz, 1H), 7.51 (dd, J=2.2, 1.0 Hz, 1H), 6.19 (dd, J=9.9, 6.8 Hz, 1H), 4.13-4.00 (m, 2H), 3.86 (ddt, J=16.4, 9.9, 1.0 Hz, 1H), 3.58 (ddt, J=16.4, 6.8, 1.0 Hz, 1H), 3.47-3.40 (m, 2H), 3.06 (t, J=6.4 Hz, 2H). [M+H] 409.0.

### Example 239

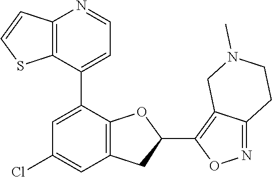 [SMILES: CN1CCc2noc([C@H]3Cc4cc(Cl)cc(-c5ccnc6ccsc56)c4O3)c2C1]

### (R)-3-(5-chloro-7-(thieno[3,2-b]pyridin-7-yl)-2,3-dihydrobenzofuran-2-yl)-5-methyl-4,5,6,7-tetrahydroisoxazolo[4,3-c]pyridine

The title compound was synthesized from reductive amination of the title compound from Example 234 using paraformaldehyde and sodium triacetoxyborohydride to give the desired product. 1H NMR (400 MHz, Methanol-d4) δ 8.84 (d, J=5.5 Hz, 1H), 8.38 (d, J=5.6 Hz, 1H), 7.81 (d, J=5.6 Hz, 1H), 7.73 (d, J=5.7 Hz, 1H), 7.64 (d, J=2.1 Hz, 1H), 7.54 (dd, J=1.3, 1.3 Hz, 1H), 6.18 (dd, J=9.9, 6.7 Hz, 1H), 4.18-4.10 (m, 2H), 3.86 (dd, J=16.4, 9.9 Hz, 1H), 3.64-3.42 (m, 3H), 3.13 (t, J=6.4 Hz, 2H), 2.81 (s, 3H). [M+H] 424.0.

### Example 240

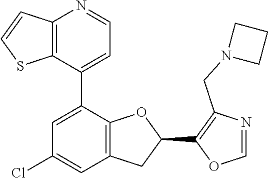 [SMILES: Clc1cc2c(c(-c3ccnc4ccsc34)c1)O[C@@H](c1ocnc1CN1CCC1)C2]

### (R)-4-(azetidin-1-ylmethyl)-5-(5-chloro-7-(thieno[3,2-b]pyridin-7-yl)-2,3-dihydrobenzofuran-2-yl)oxazole

The title compound was synthesized using precursor VII, general procedure G using 7-(4,4,5,5-tetramethyl-1,3,2-dioxaborolan-2-yl)thieno[3,2-b]pyridine to give the desired product. 1H NMR (400 MHz, Methanol-d) δ 8.64 (d, J=4.9 Hz, 1H), 8.12 (s, 1H), 8.02 (dd, J=5.6, 0.4 Hz, 1H), 7.55 (d, J=5.6 Hz, 1H), 7.51 (dd, J=2.0, 1.1 Hz, 1H), 7.48-7.43 (m, 2H), 6.21-6.12 (m, 1H), 3.74 (dd, J=16.3, 9.7 Hz, 1H), 3.64-3.55 (m, 1H), 3.57-3.44 (m, 2H), 3.24-3.14 (m, 3H), 2.01-1.93 (m, 3H). [M+H] 424.0.

### Example 241

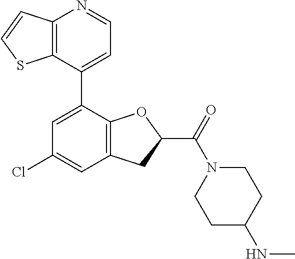 [SMILES: CNC1CCN(C(=O)[C@H]2Cc3cc(Cl)cc(-c4ccnc5ccsc45)c3O2)CC1]

### (R)-(5-chloro-7-(thieno[3,2-b]pyridin-7-yl)-2,3-dihydrobenzofuran-2-yl)(4-(methylamino)piperidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.82-8.69 (m, 1H), 7.78 (dd, J=8.9, 5.6 Hz, 1H), 7.61 (dt, J=7.3, 4.9 Hz, 1H), 7.51-7.28 (m, 3H), 5.48 (ddd, J=15.0, 9.8, 7.2 Hz, 1H), 4.79-3.82 (m, 3H), 3.38 (dt, J=17.3, 9.4 Hz, 1H), 3.16-0.83 (m, 12H). [M+H] 428.0.

### Example 242

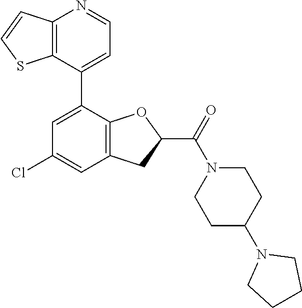 [SMILES: O=C([C@H]1Cc2cc(Cl)cc(-c3ccnc4ccsc34)c2O1)N1CCC(N2CCCC2)CC1]

### (R)-(5-chloro-7-(thieno[3,2-b]pyridin-7-yl)-2,3-dihydrobenzofuran-2-yl)(4-(pyrrolidin-1-yl)piperidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 9.11 (dd, J=237.8, 5.9 Hz, 1H), 8.38-8.20 (m, 3H), 7.50 (dd, J=47.9, 10.6 Hz, 2H), 5.68-5.40 (m, 1H), 4.75 (dd, J=33.4, 14.4 Hz, 1H), 4.07 (ddd, J=41.7, 16.5, 6.8 Hz, 2H), 3.75 (d, J=37.5 Hz, 2H), 3.61-2.48 (m, 5H), 2.48-1.81 (m, 9H). [M+H] 468.0.

### Example 243

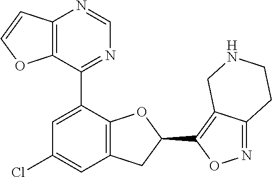 [SMILES: Clc1cc2c(c(-c3ncnc4ccoc34)c1)O[C@@H](c1onc3c1CNCC3)C2]

### (R)-3-(5-chloro-7-(furo[3,2-d]pyrimidin-4-yl)-2,3-dihydrobenzofuran-2-yl)-4,5,6,7-tetrahydroisoxazolo[4,3-c]pyridine

The title compound was synthesized using precursor III, general procedure G and D using 4-chlorofuro[3,2-d]pyrimidine to give the desired product as the HCl salt. 1H NMR (400 MHz, CD3OD) δ 9.21 (s, 1H), 8.54 (d, J=2.3 Hz, 1H), 7.95 (d, J=2.2 Hz, 1H), 7.61-7.58 (m, 1H), 7.27 (d, J=2.3 Hz, 1H), 6.28 (dd, J=10.0, 6.4 Hz, 1H), 4.50-4.48 (m, 1H), 4.35 (q, J=15.4 Hz, 2H), 3.89 (dd, J=16.4, 10.0 Hz, 1H), 3.62 (ddd, J=7.5, 5.2, 1.0 Hz, 1H), 3.53-3.48 (m, 2H), 3.12 (t, J=6.4 Hz, 1H). [M+H] 395.1.

### Example 244

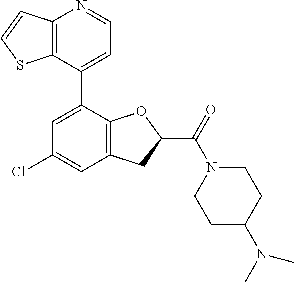 [SMILES: CN(C)C1CCN(C(=O)[C@H]2Cc3cc(Cl)cc(-c4ccnc5ccsc45)c3O2)CC1]

### (R)-(5-chloro-7-(thieno[3,2-b]pyridin-7-yl)-2,3-dihydrobenzofuran-2-yl)(4-(dimethylamino)piperidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.73 (dd, J=4.8, 1.9 Hz, 1H), 7.78 (dd, J=9.1, 5.6 Hz, 1H), 7.61 (d, J=5.6 Hz, 1H), 7.46 (d, J=2.3 Hz, 1H), 7.40 (d, J=4.8 Hz, 1H), 7.34-7.27 (m, 1H), 5.50 (dt, J=10.0, 6.7 Hz, 1H), 4.59 (t, J=15.6 Hz, 1H), 4.15-3.89 (m, 2H), 3.39 (dt, J=16.6, 8.9 Hz, 1H), 3.04-2.87 (m, 1H), 2.81-2.44 (m, 3H), 2.38 (s, 3H), 1.94 (t, J=14.4 Hz, 2H), 1.58-1.18 (m, 3H), 0.99-0.81 (m, 1H). [M+H] 442.0.

### Example 245

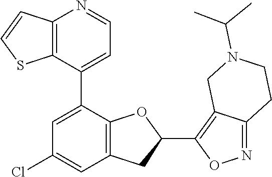 [SMILES: CC(C)N1CCc2noc([C@H]3Cc4cc(Cl)cc(-c5ccnc6ccsc56)c4O3)c2C1]

### (R)-3-(5-chloro-7-(thieno[3,2-b]pyridin-7-yl)-2,3-dihydrobenzofuran-2-yl)-5-isopropyl-4,5,6,7-tetrahydroisoxazolo[4,3-c]pyridine

The title compound was synthesized from reductive amination of the title compound from Example 234 using acetone and sodium triacetoxyborohydride to give the desired product. 1H NMR (400 MHz, Methanol-d4): ppm δ 8.75 (d, J=5.0 Hz, 1H), 8.15 (d, J=5.6 Hz, 1H), 7.67-7.63 (m, 3H), 7.51 (d, J=2.1 Hz, 1H), 6.25 (dd, J=10.0, 5.9 Hz, 1H), 4.05 (d, J=14.6 Hz, 1H), 3.91 (dd, J=16.4, 10.0 Hz, 1H), 3.61 (dd, J=16.4, 6.0 Hz, 1H), 3.21-3.32 (m, 6H), 1.02 (d, J=2.1 Hz, 6H). [M+H] 452.0.

### Example 246

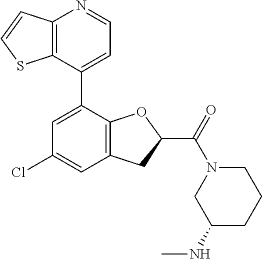 [SMILES: CN[C@H]1CCCN(C(=O)[C@H]2Cc3cc(Cl)cc(-c4ccnc5ccsc45)c3O2)C1]

### ((R)-5-chloro-7-(thieno[3,2-b]pyridin-7-yl)-2,3-dihydrobenzofuran-2-yl)((S)-3-(methylamino)piperidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 9.89-9.25 (m, 1H), 8.73 (t, J=4.6 Hz, 1H), 7.78 (t, J=5.7 Hz, 1H), 7.62 (d, J=5.6 Hz, 1H), 7.52-7.27 (m, 2H), 5.75-5.41 (m, 1H), 4.23-3.16 (m, 4H), 3.17-2.51 (m, 5H), 2.07 (d, J=79.2 Hz, 1H), 1.79-1.14 (m, 4H). [M+H] 428.0.

### Example 247

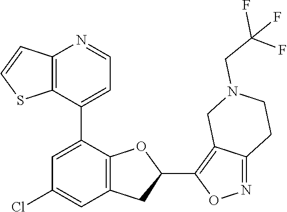 [SMILES: FC(F)(F)CN1CCc2noc([C@H]3Cc4cc(Cl)cc(-c5ccnc6ccsc56)c4O3)c2C1]

### (R)-3-(5-chloro-7-(thieno[3,2-b]pyridin-7-yl)-2,3-dihydrobenzofuran-2-yl)-5-(2,2,2-trifluoroethyl)-4,5,6,7-tetrahydroisoxazolo[4,3-c]pyridine

The title compound was synthesized from alkylation of the title compound from Example 234 using 2,2,2-trifluoroethyltrifluoromethanesulfonate and potassium carbonate to give the desired product. 1H NMR (400 MHz, Methanol-d4) δ 8.79 (s, 1H), 8.26 (d, J=5.5 Hz, 1H), 7.72 (d, J=5.2 Hz, 1H), 7.68 (d, J=5.7 Hz, 1H), 7.63 (s, 1H), 7.51 (s, 1H), 6.14 (dd, J=9.9, 5.9 Hz, 1H), 3.83 (dd, J=16.5, 9.8 Hz, 1H), 3.59-3.43 (m, 2H), 3.36 (d, J=14.6 Hz, 1H), 3.12-2.95 (m, 1H), 2.94-2.81 (m, 3H), 2.77 (t, J=5.8 Hz, 2H). [M+H] 492.0.

### Example 248

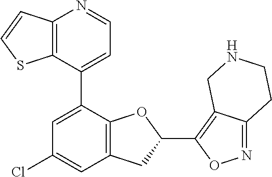 [SMILES: Clc1cc2c(c(-c3ccnc4ccsc34)c1)O[C@H](c1onc3c1CNCC3)C2]

### (S)-3-(5-chloro-7-(thieno[3,2-b]pyridin-7-yl)-2,3-dihydrobenzofuran-2-yl)-4,5,6,7-tetrahydroisoxazolo[4,3-c]pyridine

The title compound was synthesized using the enantiomer of precursor III, general procedure G and E using 7-(4,4,5,5-tetramethyl-1,3,2-dioxaborolan-2-yl)thieno[3,2-b]pyridine to give the desired product as the TFA salt. 1H NMR (400 MHz, Methanol-d4) δ 8.74 (d, J=5.2 Hz, 1H), 8.19 (d, J=5.6 Hz, 1H), 7.66-7.62 (m, 2H), 7.60 (d, J=2.1 Hz, 1H), 7.53-7.44 (m, 1H), 6.19 (dd, J=9.9, 6.8 Hz, 1H), 4.13-4.00 (m, 2H), 3.87 (dd, J=16.4, 9.9 Hz, 1H), 3.58 (dd, J=16.4, 6.8 Hz, 1H), 3.44 (t, J=6.4 Hz, 2H), 3.06 (t, J=6.4 Hz, 2H). [M+H] 410.1.

### Example 249

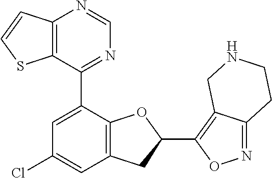 [SMILES: Clc1cc2c(c(-c3ncnc4ccsc34)c1)O[C@@H](c1onc3c1CNCC3)C2]

### (R)-3-(5-chloro-7-(thieno[3,2-d]pyrimidin-4-yl)-2,3-dihydrobenzofuran-2-yl)-4,5,6,7-tetrahydroisoxazolo[4,3-c]pyridine

The title compound was synthesized using precursor III, general procedure G and D using 4-chlorothieno[3,2-d]pyrimidine to give the desired product as the HCl salt. 1H NMR (400 MHz, CD3OD) δ 9.30 (s, 1H), 8.51 (d, J=5.5 Hz, 1H), 7.73 (d, J=2.9 Hz, 1H), 7.69 (d, J=5.5 Hz, 1H), 7.59 (d, J=1.1 Hz, 1H), 6.26 (dd, J=10.4, 5.2 Hz, 1H), 4.27 (s, 1H), 3.91 (dd, J=16.5, 10.0 Hz, 1H), 3.65-3.56 (m, 2H), 3.50 (t, J=6.3 Hz, 2H), 3.11 (t, J=6.4 Hz, 2H) [M+H] 411.1.

### Example 250

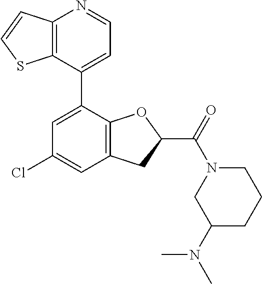 [SMILES: CN(C)C1CCCN(C(=O)[C@H]2Cc3cc(Cl)cc(-c4ccnc5ccsc45)c3O2)C1]

### ((R)-5-chloro-7-(thieno[3,2-b]pyridin-7-yl)-2,3-dihydrobenzofuran-2-yl)(3-(dimethylamino)piperidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.86-8.70 (m, 1H), 7.99-7.41 (m, 4H), 7.34 (d, J=9.4 Hz, 1H), 5.78-5.42 (m, 1H), 4.66-3.55 (m, 3H), 3.45 (q, J=14.7, 13.1 Hz, 2H), 2.92-2.75 (m, 4H), 2.19-1.59 (m, 8H). [M+H] 442.0.

### Example 251

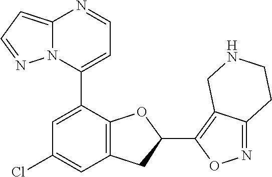 [SMILES: Clc1cc2c(c(-c3ccnc4ccnn34)c1)O[C@@H](c1onc3c1CNCC3)C2]

### (R)-3-(5-chloro-7-(pyrazolo[1,5-a]pyrimidin-7-yl)-2,3-dihydrobenzofuran-2-yl)-4,5,6,7-tetrahydroisoxazolo[4,3-c]pyridine

The title compound was synthesized using precursor III, general procedure G, E and F using 7-chloropyrazolo[1,5-a]pyrimidine to give the desired product. 1H NMR (400 MHz, CD3OD): δ ppm 8.51 (d, J=5.6 Hz, 1H), 8.16 (d, J=2.4 Hz, 1H), 7.96 (d, J=2.4 Hz, 1H), 7.37 (bs, 1H), 7.04 (d, J=5.6 Hz, 1H), 6.78 (s, 1H), 5.95 (dd, J=9.6, 6.4 Hz, 1H), 3.75-3.51 (m, 4H), 3.01 (t, J=6.0 Hz, 2H), 2.76 (t, J=6.0 Hz, 2H). [M+H] 394.0.

### Example 252

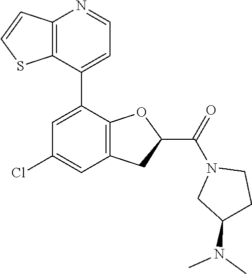 [SMILES: CN(C)[C@@H]1CCN(C(=O)[C@H]2Cc3cc(Cl)cc(-c4ccnc5ccsc45)c3O2)C1]

### ((R)-5-chloro-7-(thieno[3,2-b]pyridin-7-yl)-2,3-dihydrobenzofuran-2-yl)((R)-3-(dimethylamino)pyrrolidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.74 (dd, J=4.8, 1.7 Hz, 1H), 7.87 (dd, J=70.7, 5.6 Hz, 1H), 7.61 (dd, J=5.6, 2.3 Hz, 1H), 7.46-7.28 (m, 3H), 5.36 (ddd, J=25.6, 10.0, 6.8 Hz, 1H), 4.04-2.97 (m, 7H), 2.53 (d, J=32.7 Hz, 6H), 2.37-0.79 (m, 2H). [M+H] 428.0.

### Example 253

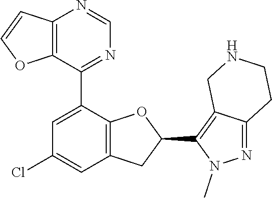 [SMILES: Cn1nc2c(c1[C@H]1Cc3cc(Cl)cc(-c4ncnc5ccoc45)c3O1)CNCC2]

### (R)-4-(5-chloro-2-(2-methyl-4,5,6,7-tetrahydro-2H-pyrazolo[4,3-c]pyridin-3-yl)-2,3-dihydrobenzofuran-7-yl)furo[3,2-d]pyrimidine

The title compound was synthesized using precursor IV, general procedure G and D using 4-chlorofuro[3,2-d]pyrimidine to give the desired product as the HCl salt. 1H NMR (400 MHz, CD3OD) δ 9.00 (s, 1H), 8.75 (s, 1H), 8.35 (d, J=2.3 Hz, 1H), 7.72 (d, J=2.3 Hz, 1H), 7.40 (d, J=2.2 Hz, 1H), 6.02-5.78 (m, 1H), 4.14-3.96 (m, 2H), 3.63 (d, J=2.8 Hz, 4H), 3.44 (dd, J=10.8, 6.1 Hz, 1H), 3.35 (q, J=7.0 Hz, 2H), 2.88 (d, J=6.3 Hz, 2H). [M+H] 408.1.

### Example 254

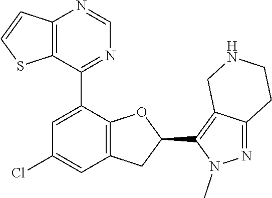 [SMILES: Cn1nc2c(c1[C@H]1Cc3cc(Cl)cc(-c4ncnc5ccsc45)c3O1)CNCC2]

### (R)-4-(5-chloro-2-(2-methyl-4,5,6,7-tetrahydro-2H-pyrazolo[4,3-c]pyridin-3-yl)-2,3-dihydrobenzofuran-7-yl)thieno[3,2-d]pyrimidine

The title compound was synthesized using precursor IV, general procedure G and D using 4-chlorothieno[3,2-d]pyrimidine to give the desired product as the HCl salt. 1H NMR (400 MHz, CD3OD) δ 9.15 (s, 1H), 8.42 (d, J=5.5 Hz, 1H), 7.56 (dd, J=3.9, 1.7 Hz, 2H), 7.47-7.39 (m, 1H), 5.94 (dd, J=9.4, 7.7 Hz, 1H), 4.04-3.88 (m, 2H), 3.62 (s, 4H), 3.53 (d, J=5.7 Hz, 2H), 3.34 (dq, J=12.6, 6.4 Hz, 1H), 2.87 (t, J=6.2 Hz, 2H). [M+H] 423.1.

### Example 255

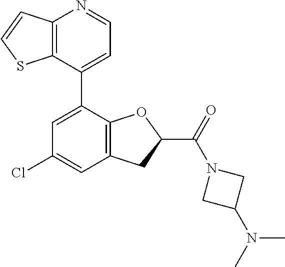 [SMILES: CN(C)C1CN(C(=O)[C@H]2Cc3cc(Cl)cc(-c4ccnc5ccsc45)c3O2)C1]

### (R)-(5-chloro-7-(thieno[3,2-b]pyridin-7-yl)-2,3-dihydrobenzofuran-2-yl)(3-(dimethylamino)azetidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt.

### Example 256

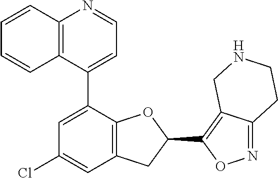 [SMILES: Clc1cc2c(c(-c3ccnc4ccccc34)c1)O[C@@H](c1onc3c1CNCC3)C2]

### (R)-3-(5-chloro-7-(quinolin-4-yl)-2,3-dihydrobenzofuran-2-yl)-4,5,6,7-tetrahydroisoxazolo[4,3-c]pyridine

The title compound was synthesized using precursor III, general procedure C and D using quinoline-4-boronic acid to give the desired product as the HCl salt. 1H NMR (400 MHz, CD3OD): δ ppm 9.29 (d, J=5.7 Hz, 1H), 8.33 (d, J=8.5 Hz, 1H), 8.21 (ddd, J=8.5, 7.0, 1.1 Hz, 1H), 8.17-8.10 (m, 2H), 7.95 (t, J=7.7 Hz, 1H), 7.65-7.58 (m, 1H), 7.50 (d, J=2.1 Hz, 1H), 6.17 (dd, J=9.9, 6.5 Hz, 1H), 4.17-4.00 (m, 2H), 4.00-3.82 (m, 1H), 3.53-3.10 (m, 5H).

### Example 257

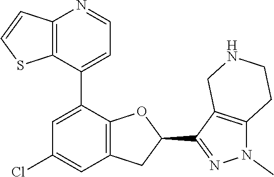 [SMILES: Cn1nc([C@H]2Cc3cc(Cl)cc(-c4ccnc5ccsc45)c3O2)c2c1CCNC2]

### (R)-7-(5-chloro-2-(1-methyl-4,5,6,7-tetrahydro-1H-pyrazolo[4,3-c]pyridin-3-yl)-2,3-dihydrobenzofuran-7-yl)thieno[3,2-b]pyridine

The title compound was synthesized using precursor IV, general procedure C and D using 7-chloro-2-(((2,3,3-trimethylbutan-2-yl)oxy)methyl)thieno[3,2-b]pyridine to give the desired product as the HCl salt. 1H NMR (400 MHz, Methanol-d4) δ 8.81 (d, J=5.8 Hz, 1H), 8.42 (dd, J=5.7, 0.4 Hz, 1H), 7.86 (dd, J=5.8, 0.4 Hz, 1H), 7.71 (d, J=5.7 Hz, 1H), 7.61 (d, J=2.2 Hz, 1H), 7.54-7.42 (m, 1H), 6.16 (dd, J=9.7, 8.3 Hz, 1H), 4.05 (d, J=14.7 Hz, 1H), 3.90 (d, J=14.7 Hz, 1H), 3.81 (ddt, J=16.5, 9.8, 1.0 Hz, 1H), 3.75 (s, 3H), 3.38 (dt, J=8.4, 1.2 Hz, 1H), 3.33 (td, J=6.3, 1.6 Hz, 3H), 2.84 (t, J=6.3 Hz, 2H). [M+H] 423.0.

### Example 258

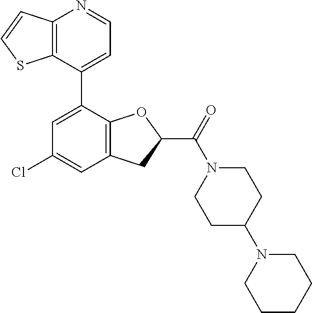 [SMILES: O=C([C@H]1Cc2cc(Cl)cc(-c3ccnc4ccsc34)c2O1)N1CCC(N2CCCCC2)CC1]

### (R)-[1,4′-bipiperidin]-1′-yl(5-chloro-7-(thieno[3,2-b]pyridin-7-yl)-2,3-dihydrobenzofuran-2-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.73 (dd, J=4.8, 1.8 Hz, 1H), 7.79 (dd, J=15.5, 5.6 Hz, 1H), 7.62 (t, J=5.3 Hz, 1H), 7.46 (d, J=2.4 Hz, 1H), 7.40 (d, J=4.8 Hz, 1H), 7.33-7.28 (m, 1H), 5.51 (ddd, J=16.4, 10.0, 6.8 Hz, 1H), 4.61 (d, J=13.6 Hz, 1H), 4.17-3.86 (m, 2H), 3.39 (dd, J=16.3, 9.7 Hz, 1H), 2.98-0.72 (m, 17H). [M+H] 482.0.

### Example 259

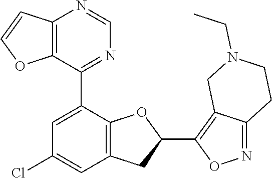 [SMILES: CCN1CCc2noc([C@H]3Cc4cc(Cl)cc(-c5ncnc6ccoc56)c4O3)c2C1]

### (R)-3-(5-chloro-7-(furo[3,2-d]pyrimidin-4-yl)-2,3-dihydrobenzofuran-2-yl)-5-ethyl-4,5,6,7-tetrahydroisoxazolo[4,3-c]pyridine

The title compound was synthesized from reductive amination of the title compound from Example 243 using acetaldehyde and sodium triacetoxyborohydride to give the desired product. 1H NMR (400 MHz, CD3OD) δ 8.93 (s, 1H), 8.21 (d, J=2.3 Hz, 1H), 7.67 (d, J=2.4 Hz, 1H), 7.45-7.31 (m, 1H), 7.01 (d, J=2.3 Hz, 1H), 6.04 (dd, J=9.9, 5.6 Hz, 1H), 3.69 (ddt, J=16.3, 9.9, 1.2 Hz, 1H), 3.38 (dd, J=16.3, 5.5 Hz, 1H), 3.26 (d, J=14.5 Hz, 1H), 3.05 (d, J=14.5 Hz, 1H), 2.67 (t, J=5.5 Hz, 2H), 2.56 (ddt, J=24.4, 12.2, 6.6 Hz, 2H), 2.38-2.18 (m, 2H), 0.79 (t, J=7.2 Hz, 3H). [M+H] 423.1.

### Example 260

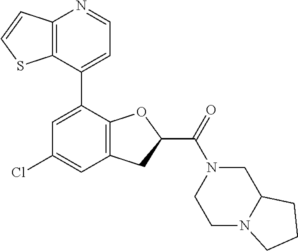 [SMILES: O=C([C@H]1Cc2cc(Cl)cc(-c3ccnc4ccsc34)c2O1)N1CCN2CCCC2C1]

### ((R)-5-chloro-7-(thieno[3,2-b]pyridin-7-yl)-2,3-dihydrobenzofuran-2-yl)(hexahydropyrrolo[1,2-a]pyrazin-2(1H)-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.72 (d, J=4.8 Hz, 1H), 7.78 (dd, J=5.6, 3.0 Hz, 1H), 7.62 (dd, J=7.9, 5.6 Hz, 1H), 7.51-7.28 (m, 3H), 5.48 (ddd, J=16.5, 9.9, 6.6 Hz, 1H), 4.52 (d, J=13.4 Hz, 1H), 4.11-3.78 (m, 2H), 3.50-2.44 (m, 6H), 2.26-1.01 (m, 6H). [M+H] 440.0.

### Example 261

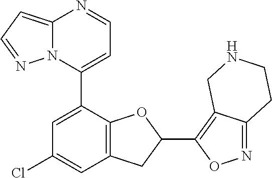 [SMILES: Clc1cc2c(c(-c3ccnc4ccnn34)c1)OC(c1onc3c1CNCC3)C2]

### 3-(5-chloro-7-(pyrazolo[1,5-a]pyrimidin-7-yl)-2,3-dihydrobenzofuran-2-yl)-4,5,6,7-tetrahydroisoxazolo[4,3-c]pyridine

The title compound was synthesized using precursor III, general procedure G and D using 7-chloropyrazolo[1,5-a]pyrimidine to give the desired product as the HCl salt. 1H NMR (400 MHz, CD3OD): δ ppm 8.58 (d, J=4.4 Hz, 1H), 8.21 (d, J=2.4 Hz, 1H), 7.87 (d, J=2.3 Hz, 1H), 7.56-7.54 (m, 1H), 7.22 (d, J=4.3 Hz, 1H), 6.82 (d, J=2.4 Hz, 1H), 6.18 (dd, J=9.9, 6.7 Hz, 1H), 4.18-4.04 (m, 2H), 3.92-3.83 (m, 1H), 3.61-3.52 (m, 1H), 3.49-3.44 (m, 2H), 3.11-3.06 (m, 2H). [M+H] 394.0.

### Example 262

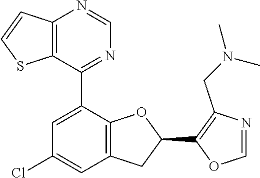 [SMILES: CN(C)Cc1ncoc1[C@H]1Cc2cc(Cl)cc(-c3ncnc4ccsc34)c2O1]

### (R)-1-(5-(5-chloro-7-(thieno[3,2-d]pyrimidin-4-yl)-2,3-dihydrobenzofuran-2-yl)oxazol-4-yl)-N,N-dimethylmethanamine

The title compound was synthesized using precursor VII, general procedure G using 4-chlorothieno[3,2-d]pyrimidine to give the desired product. 1H NMR (400 MHz, Methanol-d) δ 9.17 (s, 1H), 8.31 (d, J=5.6 Hz, 1H), 8.18 (d, J=0.6 Hz, 1H), 7.63-7.61 (m, 1H), 7.57 (d, J=5.6 Hz, 1H), 7.54-7.52 (m, 1H), 6.24 (ddd, J=9.8, 7.4, 0.5 Hz, 1H), 3.76 (dd, J=16.4, 9.8 Hz, 1H), 3.67-3.58 (m, 1H), 3.53 (d, J=13.7 Hz, 1H), 3.45 (d, J=13.7 Hz, 1H), 2.19 (s, 6H). [M+H] 413.0.

### Example 263

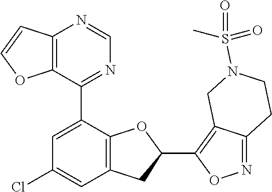 [SMILES: CS(=O)(=O)N1CCc2noc([C@H]3Cc4cc(Cl)cc(-c5ncnc6ccoc56)c4O3)c2C1]

### (R)-3-(5-chloro-7-(furo[3,2-d]pyrimidin-4-yl)-2,3-dihydrobenzofuran-2-yl)-5-(methylsulfonyl)-4,5,6,7-tetrahydroisoxazolo[4,3-c]pyridine

The title compound was synthesized by treatment of the title compound from Example 243 with methanesulfonyl chloride to give the desired product. 1H NMR (400 MHz, CD3OD) δ 9.11 (s, 1H), 8.64-8.11 (m, 2H), 7.96-7.32 (m, 1H), 7.11 (dd, J=21.4, 2.3 Hz, 1H), 6.29-6.04 (m, 1H), 3.92-3.73 (m, 2H), 3.65-3.46 (m, 2H), 2.88 (t, J=6.0 Hz, 3H), 2.84 (s, 1H), 2.74 (s, 3H). [M+H] 473.1.

### Example 264

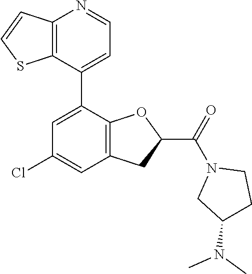 [SMILES: CN(C)[C@H]1CCN(C(=O)[C@H]2Cc3cc(Cl)cc(-c4ccnc5ccsc45)c3O2)C1]

### ((R)-5-chloro-7-(thieno[3,2-b]pyridin-7-yl)-2,3-dihydrobenzofuran-2-yl)((S)-3-(dimethylamino)pyrrolidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.74 (dd, J=15.4, 4.9 Hz, 1H), 7.92-7.27 (m, 5H), 5.40 (ddd, J=10.0, 7.0, 4.8 Hz, 1H), 4.08-3.59 (m, 3H), 3.44 (td, J=16.8, 16.3, 10.8 Hz, 4H), 3.12-2.63 (m, 2H), 2.42 (s, 3H), 2.13 (s, 3H). [M+H] 428.0.

### Example 265

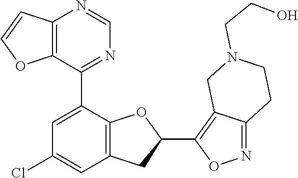 [SMILES: OCCN1CCc2noc([C@H]3Cc4cc(Cl)cc(-c5ncnc6ccoc56)c4O3)c2C1]

### (R)-2-(3-(5-chloro-7-(furo[3,2-d]pyrimidin-4-yl)-2,3-dihydrobenzofuran-2-yl)-6,7-dihydroisoxazolo[4,3-c]pyridin-5(4H)-yl)ethan-1-ol

The title compound was synthesized by treatment of the title compound from Example 243 with potassium carbonate and 2-bromoethanol to give the desired product. 1H NMR (400 MHz, CD3OD) δ 8.92 (s, 1H), 7.86 (d, J=2.2 Hz, 1H), 7.47 (d, J=2.2 Hz, 1H), 7.45-7.38 (m, 1H), 6.19 (dd, J=10.0, 6.5 Hz, 1H), 3.76 (td, J=15.5, 15.1, 10.5 Hz, 4H), 3.45 (tt, J=18.0, 8.7 Hz, 4H), 3.32-3.20 (m, 1H), 3.10 (d, J=6.5 Hz, 5H). [M+H] 439.1.

### Example 266

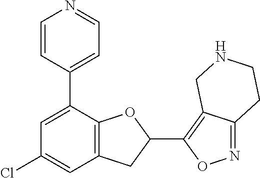 [SMILES: Clc1cc2c(c(-c3ccncc3)c1)OC(c1onc3c1CNCC3)C2]

### 3-(5-chloro-7-(pyridin-4-yl)-2,3-dihydrobenzofuran-2-yl)-4,5,6,7-tetrahydroisoxazolo[4,3-c]pyridine

The title compound was synthesized using precursor III, general procedure C and E using potassium trifluoro(4-pyridyl)borate to give the desired product as the TFA salt. ¹H NMR (400 MHz, DMSO-d6) δ 9.08-8.87 (m, 1H), 8.83-8.60 (m, 2H), 7.98-7.75 (m, 2H), 7.69 (s, 1H), 7.53 (s, 1H), 6.43-6.21 (m, 1H), 4.21-4.07 (m, 2H), 3.49-3.29 (m, 4H), 3.08-2.91 (m, 2H). [M+H] 354.1.

### Example 267

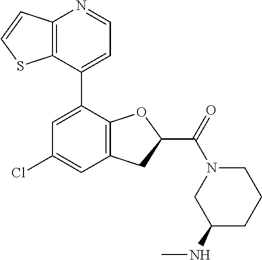 [SMILES: CN[C@@H]1CCCN(C(=O)[C@H]2Cc3cc(Cl)cc(-c4ccnc5ccsc45)c3O2)C1]

### ((R)-5-chloro-7-(thieno[3,2-b]pyridin-7-yl)-2,3-dihydrobenzofuran-2-yl)((R)-3-(methylamino)piperidin-1-yl)methanone

The title compound was synthesized using general procedure A, C and D using 7-chloro-2-(((2,3,3-trimethylbutan-2-yl)oxy)methyl)thieno[3,2-b]pyridine to give the desired product as the HCl salt. 1H NMR (400 MHz, Chloroform-d) δ 8.75 (dd, J=13.5, 4.9 Hz, 1H), 7.84-7.74 (m, 1H), 7.65-7.58 (m, 1H), 7.50-7.23 (m, 3H), 5.52 (td, J=10.0, 6.8 Hz, 1H), 4.28 (dd, J=36.2, 12.7 Hz, 1H), 4.12-3.61 (m, 2H), 3.39 (ddd, J=25.5, 16.3, 9.8 Hz, 1H), 3.22-2.59 (m, 2H), 2.52 (d, J=9.6 Hz, 1H), 2.24-1.32 (m, 6H). [M+H] 428.0.

### Example 268

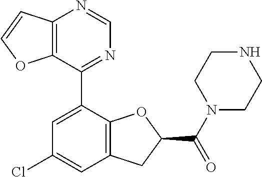 [SMILES: O=C([C@H]1Cc2cc(Cl)cc(-c3ncnc4ccoc34)c2O1)N1CCNCC1]

### (R)-(5-chloro-7-(furo[3,2-d]pyrimidin-4-yl)-2,3-dihydrobenzofuran-2-yl)(piperazin-1-yl)methanone

The title compound was synthesized using precursor II, general procedure B and D using 4-chlorofuro[3,2-d]pyrimidine to give the desired product as the HCl salt. 1H NMR (400 MHz, CD3OD) δ 8.48 (d, J=2.1 Hz, 1H), 7.85 (d, J=2.2 Hz, 1H), 7.66 (d, J=7.3 Hz, 1H), 7.55-7.49 (m, 1H), 7.23 (d, J=2.2 Hz, 1H), 5.85 (dd, J=10.0, 7.1 Hz, 1H), 4.07 (d, J=13.2 Hz, 2H), 3.94-3.74 (m, 2H), 3.65 (s, 2H), 3.58-3.42 (m, 2H), 3.23-3.08 (m, 2H). [M+H] 385.1.

### Example 269

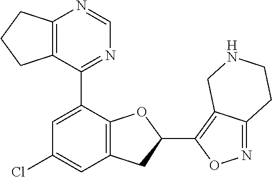 [SMILES: Clc1cc2c(c(-c3ncnc4c3CCC4)c1)O[C@@H](c1onc3c1CNCC3)C2]

### (R)-3-(5-chloro-7-(6,7-dihydro-5H-cyclopenta[d]pyrimidin-4-yl)-2,3-dihydrobenzofuran-2-yl)-4,5,6,7-tetrahydroisoxazolo[4,3-c]pyridine

The title compound was synthesized using precursor III, general procedure G and D using 4-chloro-6,7-dihydro-5H-cyclopenta[d]pyrimidine to give the desired product as the HCl salt. 1H NMR (400 MHz, Chloroform-d) δ 9.04 (s, 1H), 7.42 (d, J=2.2 Hz, 1H), 7.29 (d, J=2.0 Hz, 1H), 5.96 (dd, J=9.8, 6.3 Hz, 1H), 3.78-3.44 (m, 4H), 3.16-2.72 (m, 8H), 2.10 (dddd, J=18.7, 15.5, 10.3, 6.8 Hz, 2H). [M+H] 395.0.

### Example 270

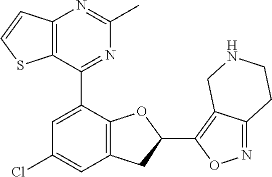 [SMILES: Cc1nc(-c2cc(Cl)cc3c2O[C@@H](c2onc4c2CNCC4)C3)c2sccc2n1]

### (R)-3-(5-chloro-7-(2-methylthieno[3,2-d]pyrimidin-4-yl)-2,3-dihydrobenzofuran-2-yl)-4,5,6,7-tetrahydroisoxazolo[4,3-c]pyridine

The title compound was synthesized using precursor III, general procedure G and D using 4-chloro-2-methylthieno[3,2-d]pyrimidine to give the desired product as the HCl salt. 1H NMR (400 MHz, CD3OD) δ 8.89 (d, J=5.5 Hz, 1H), 7.95 (S, 1H), 7.90-7.75 (m, 2H), 6.49 (dd, J=10.0, 6.7 Hz, 1H), 4.42 (dd, J=38.7, 15.4 Hz, 2H), 4.08 (dd, J=16.6, 10.1 Hz, 1H), 3.82-3.76 (m, 1H), 3.68 (t, J=6.3 Hz, 2H), 3.30 (t, J=6.4 Hz, 2H), 3.16 (s, 3H). [M+H] 425.1.

### Example 271

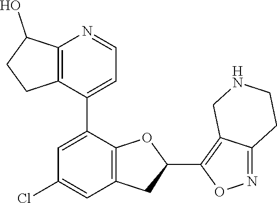 [SMILES: OC1CCc2c(-c3cc(Cl)cc4c3O[C@@H](c3onc5c3CNCC5)C4)ccnc21]

### 4-((R)-5-chloro-2-(4,5,6,7-tetrahydroisoxazolo[4,3-c]pyridin-3-yl)-2,3-dihydrobenzofuran-7-yl)-6,7-dihydro-5H-cyclopenta[b]pyridin-7-ol

The title compound was synthesized using precursor III, general procedure C and D using 4-(4,4,5,5-tetramethyl-1,3,2-dioxaborolan-2-yl)-6,7-dihydro-5H-cyclopenta[b]pyridin-7-ol to give the desired product as the HCl salt. 1H NMR (400 MHz, Methanol-d4) δ 8.67 (s, 1H), 8.10-7.91 (m, 1H), 7.55 (s, 1H), 7.47 (dd, J=4.5, 2.1 Hz, 1H), 6.33-6.17 (m, 1H), 5.61-5.41 (m, 1H), 4.22-4.00 (m, 1H), 3.96-3.79 (m, 1H), 3.65 (s, 2H), 3.63-3.43 (m, 2H), 3.28-3.17 (m, 1H), 3.17-3.07 (m, 2H), 3.07-2.93 (m, 1H), 2.82-2.56 (m, 1H), 2.16-1.93 (m, 1H). [M+H] 410.0.

### Example 272

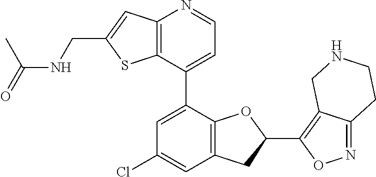 [SMILES: CC(=O)NCc1cc2nccc(-c3cc(Cl)cc4c3O[C@@H](c3onc5c3CNCC5)C4)c2s1]

### (R)-N-((7-(5-chloro-2-(4,5,6,7-tetrahydroisoxazolo[4,3-c]pyridin-3-yl)-2,3-dihydrobenzofuran-7-yl)thieno[3,2-b]pyridin-2-yl)methyl)acetamide

The title compound was synthesized using precursor III, general procedure C and D using N-((7-(4,4,5,5-tetramethyl-1,3,2-dioxaborolan-2-yl)thieno[3,2-b]pyridin-2-yl)methyl)acetamide to give the desired product as the HCl salt. ¹H NMR (400 MHz, CD3OD): δ ppm 8.80 (s, 1H), 7.78 (d, J=5.8 Hz, 1H), 7.59 (d, J=4.3 Hz, 2H), 6.21 (s, 1H), 4.80-4.68 (m, 2H), 4.17 (s, 1H), 3.97-3.81 (m, 1H), 3.52 (dt, J=14.0, 6.1 Hz, 4H), 3.12 (dd, J=8.0, 4.8 Hz, 2H), 2.03 (s, 3H).

### Example 273

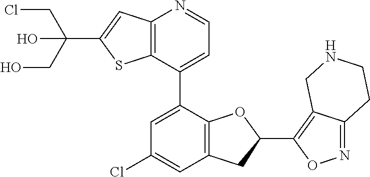 [SMILES: OCC(O)(CCl)c1cc2nccc(-c3cc(Cl)cc4c3O[C@@H](c3onc5c3CNCC5)C4)c2s1]

### 3-chloro-2-(7-((R)-5-chloro-2-(4,5,6,7-tetrahydroisoxazolo[4,3-c]pyridin-3-yl)-2,3-dihydrobenzofuran-7-yl)thieno[3,2-b]pyridin-2-yl)propane-1,2-diol

The title compound was synthesized using precursor IV, general procedure C and D using 3-(7-(4,4,5,5-tetramethyl-1,3,2-dioxaborolan-2-yl)thieno[3,2-b]pyridin-2-yl)oxetan-3-ol to give the desired product as the HCl salt. ¹H NMR (400 MHz, CD3OD): δ ppm 8.84 (bs, 1H), 7.93 (bs, 1H), 7.71 (s, 1H), 7.64 (s, 1H), 7.58 (s, 1H), 6.20 (bs, 1H), 4.15-3.50 (m, 10H), 3.09 (bs, 2H). [M+H] 517.9.

### Example 274

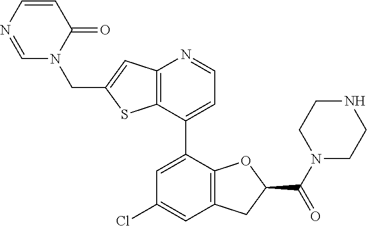 [SMILES: O=C([C@H]1Cc2cc(Cl)cc(-c3ccnc4cc(Cn5cnccc5=O)sc34)c2O1)N1CCNCC1]

### (R)-3-((7-(5-chloro-2-(piperazine-1-carbonyl)-2,3-dihydrobenzofuran-7-yl)thieno[3,2-b]pyridin-2-yl)methyl)pyrimidin-4(3H)-one

The title compound was synthesized using precursor II, general procedure B and D using 3-((7-chlorothieno[3,2-b]pyridin-2-yl)methyl)pyrimidin-4(3H)-one to give the desired product as the HCl salt. 1H NMR (400 MHz, Methanol-d4) δ 9.14 (s, 1H), 8.97 (d, J=6.0 Hz, 1H), 8.18 (d, J=6.1 Hz, 1H), 8.06 (d, J=7.0 Hz, 1H), 7.95 (s, 1H), 7.65 (d, J=2.1 Hz, 1H), 7.55 (dd, J=2.1, 1.1 Hz, 1H), 6.67 (d, J=6.9 Hz, 1H), 5.91 (dd, J=9.7, 7.0 Hz, 1H), 5.70 (s, 2H), 3.93-3.56 (m, 8H), 3.27-3.22 (m, 2H). [M+H] 508.0.

### Example 275

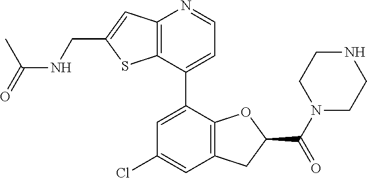 [SMILES: CC(=O)NCc1cc2nccc(-c3cc(Cl)cc4c3O[C@@H](C(=O)N3CCNCC3)C4)c2s1]

### (R)-N-((7-(5-chloro-2-(piperazine-1-carbonyl)-2,3-dihydrobenzofuran-7-yl)thieno[3,2-b]pyridin-2-yl)methyl)acetamide

The title compound was synthesized using precursor II, general procedure B and D using N-((7-chlorothieno[3,2-b]pyridin-2-yl)methyl)acetamide to give the desired product as the HCl salt. 1H NMR (400 MHz, CD3OD): δ ppm 8.93 (bs, 1H), 8.12 (s, 1H), 7.72 (s, 1H), 7.63 (s, 1H), 7.54 (s, 1H), 5.90 (bs, 1H), 4.80 (s, 2H), 3.95-3.12 (m, 10H), 2.04 (s, 3H). [M+H] 471.0.

### Example 276

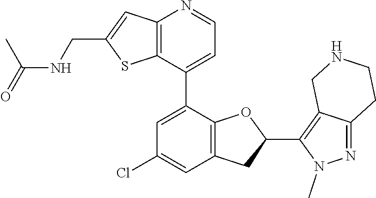 [SMILES: CC(=O)NCc1cc2nccc(-c3cc(Cl)cc4c3O[C@@H](c3c5c(nn3C)CCNC5)C4)c2s1]

### (R)-N-((7-(5-chloro-2-(2-methyl-4,5,6,7-tetrahydro-2H-pyrazolo[4,3-c]pyridin-3-yl)-2,3-dihydrobenzofuran-7-yl)thieno[3,2-b]pyridin-2-yl)methyl)acetamide

The title compound was synthesized using precursor IV, general procedure C and D using N-((7-(4,4,5,5-tetramethyl-1,3,2-dioxaborolan-2-yl)thieno[3,2-b]pyridin-2-yl)methyl)acetamide to give the desired product as the HCl salt. 1H NMR (400 MHz, Methanol-d4) δ 8.59 (d, J=5.0 Hz, 1H), 7.50-7.27 (m, 4H), 5.90 (dd, J=9.5, 7.9 Hz, 1H), 4.71-4.49 (m, 2H), 3.86-3.63 (m, 7H), 3.57 (dd, J=16.4, 8.0 Hz, 1H), 3.19 (dddd, J=18.9, 12.5, 6.4, 6.4 Hz, 2H), 2.79 (t, J=6.0 Hz, 2H), 2.00 (s, 3H). [M+H] 494.0.

### Example 277

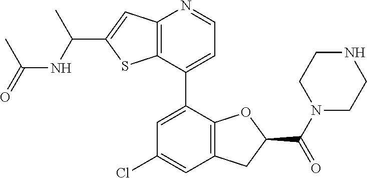 [SMILES: CC(=O)NC(C)c1cc2nccc(-c3cc(Cl)cc4c3O[C@@H](C(=O)N3CCNCC3)C4)c2s1]

### N-(1-(7-((R)-5-chloro-2-(piperazine-1-carbonyl)-2,3-dihydrobenzofuran-7-yl)thieno[3,2-b]pyridin-2-yl)ethyl)acetamide

The title compound was synthesized using precursor II, general procedure B and D using N-(1-(7-chlorothieno[3,2-b]pyridin-2-yl)ethyl)acetamide to give the desired product as the HCl salt. 1H NMR (400 MHz, CD3OD): δ ppm 8.91 (d, J=11.2 Hz, 1H), 8.11 (d, J=14.6 Hz, 1H), 7.69 (d, J=15.3 Hz, 1H), 7.67-7.59 (m, 1H), 7.54 (d, J=5.5 Hz, 1H), 5.90 (s, 1H), 5.57-5.22 (m, 1H), 3.86 (d, J=23.6 Hz, 2H), 3.79-3.39 (m, 6H), 3.19 (d, J=48.6 Hz, 2H), 2.03 (s, 3H), 1.78-1.54 (m, 3H). [M+H] 485.0.

### Example 278

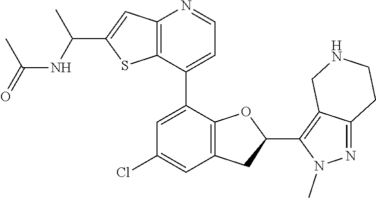 [SMILES: CC(=O)NC(C)c1cc2nccc(-c3cc(Cl)cc4c3O[C@@H](c3c5c(nn3C)CCNC5)C4)c2s1]

### N-(1-(7-((R)-5-chloro-2-(2-methyl-4,5,6,7-tetrahydro-2H-pyrazolo[4,3-c]pyridin-3-yl)-2,3-dihydrobenzofuran-7-yl)thieno[3,2-b]pyridin-2-yl)ethyl)acetamide

The title compound was synthesized using precursor IV, general procedure C and D using N-(1-(7-(4,4,5,5-tetramethyl-1,3,2-dioxaborolan-2-yl)thieno[3,2-b]pyridin-2-yl)ethyl)acetamide to give the desired product as the HCl salt. ¹H NMR (400 MHz, Methanol-d4) δ 8.59 (d, J=5.0 Hz, 1H), 7.62-7.13 (m, 4H), 5.90 (ddd, J=9.8, 7.9, 2.3 Hz, 1H), 5.42-5.21 (m, 1H), 3.90-3.41 (m, 7H), 3.41-2.97 (m, 6H), 2.80 (t, J=6.2 Hz, 2H), 1.98 (d, J=2.4 Hz, 3H), 1.61 (dd, J=7.0, 2.9 Hz, 3H). [M+H] 508.0.

### Example 279

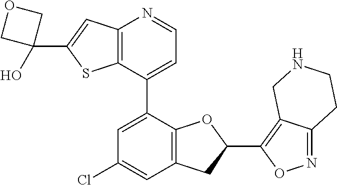 [SMILES: OC1(c2cc3nccc(-c4cc(Cl)cc5c4O[C@@H](c4onc6c4CNCC6)C5)c3s2)COC1]

### (R)-3-(7-(5-chloro-2-(4,5,6,7-tetrahydroisoxazolo[4,3-c]pyridin-3-yl)-2,3-dihydrobenzofuran-7-yl)thieno[3,2-b]pyridin-2-yl)oxetan-3-ol

The title compound was synthesized using precursor III, general procedure C and D using 3-(7-(4,4,5,5-tetramethyl-1,3,2-dioxaborolan-2-yl)thieno[3,2-b]pyridin-2-yl)oxetan-3-ol to give the desired product as the HCl salt. 1H NMR (400 MHz, CD3OD): δ ppm 8.64 (d, J=5.6 Hz, 1H), 7.67 (s, 1H), 7.49 (s, 1H), 7.45 (s, 1H), 7.43 (s, 1H), 6.07 (dd, J=10.0, 6.0 Hz, 1H), 5.48 (s, 4H), 3.83-3.29 (m, 4H), 2.95 (t, J=5.6 Hz, 2H), 2.72 (t, J=6.0 Hz, 2H). [M+H] 482.0.

### Example 280

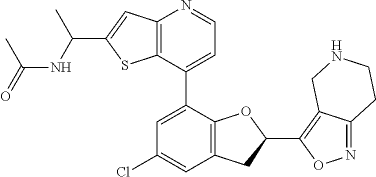 [SMILES: CC(=O)NC(C)c1cc2nccc(-c3cc(Cl)cc4c3O[C@@H](c3onc5c3CNCC5)C4)c2s1]

### N-(1-(7-((R)-5-chloro-2-(4,5,6,7-tetrahydroisoxazolo[4,3-c]pyridin-3-yl)-2,3-dihydrobenzofuran-7-yl)thieno[3,2-b]pyridin-2-yl)ethyl)acetamide

The title compound was synthesized using precursor III, general procedure G and D using N-(1-(7-chlorothieno[3,2-b]pyridin-2-yl)ethyl)acetamide to give the desired product as the HCl salt. 1H NMR (400 MHz, CD3OD): δ ppm 8.61 (d, J=4.8 Hz, 1H), 7.92 (d, J=6.8 Hz, 1H), 7.49-7.32 (m, 3H), 6.10-6.058 (m, 1H), 5.37-5.32 (m, 1H), 3.84-3.33 (m, 4H), 2.96 (t, J=5.2 Hz, 2H), 2.76-2.71 (m, 1H), 1.61 (d, J=6.8 Hz, 3H). [M+H] 495.0.

### Example 281

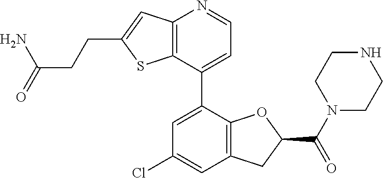 [SMILES: NC(=O)CCc1cc2nccc(-c3cc(Cl)cc4c3O[C@@H](C(=O)N3CCNCC3)C4)c2s1]

### (R)-3-(7-(5-chloro-2-(piperazine-1-carbonyl)-2,3-dihydrobenzofuran-7-yl)thieno[3,2-b]pyridin-2-yl)propenamide

The title compound was synthesized using precursor II, general procedure B using ethyl 3-(7-chlorothieno[3,2-b]pyridin-2-yl)propanoate followed by saponification, amide bond coupling with HATU, and general procedure D to give the desired product as the HCl salt. [M+H] 471.0.

### Example 282

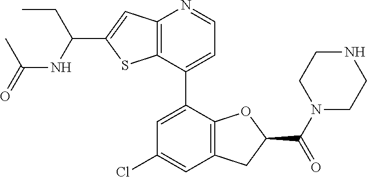 [SMILES: CCC(NC(C)=O)c1cc2nccc(-c3cc(Cl)cc4c3O[C@@H](C(=O)N3CCNCC3)C4)c2s1]

### N-(1-(7-((R)-5-chloro-2-(piperazine-1-carbonyl)-2,3-dihydrobenzofuran-7-yl)thieno[3,2-b]pyridin-2-yl)propyl)acetamide

The title compound was synthesized using precursor II, general procedure B and D using N-(1-(7-chlorothieno[3,2-b]pyridin-2-yl)propyl)acetamide to give the desired product as the HCl salt. 1H NMR (400 MHz, CD3OD): δ ppm 8.94-8.72 (m, 1H), 7.95-7.72 (m, 2H), 7.53 (d, J=32.7 Hz, 2H), 6.10-5.72 (m, 1H), 5.33-5.03 (m, 1H), 3.98-3.60 (m, 6H), 3.54 (s, 2H), 3.29-2.88 (m, 4H), 2.05 (s, 3H), 1.04 (d, J=6.5 Hz, 3H).

### Example 283

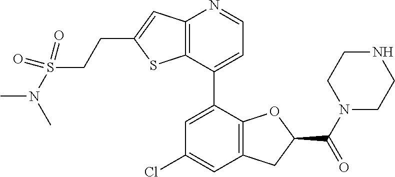 [SMILES: CN(C)S(=O)(=O)CCc1cc2nccc(-c3cc(Cl)cc4c3O[C@@H](C(=O)N3CCNCC3)C4)c2s1]

### (R)-2-(7-(5-chloro-2-(piperazine-1-carbonyl)-2,3-dihydrobenzofuran-7-yl)thieno[3,2-b]pyridin-2-yl)-N,N-dimethyl ethane-1-sulfonamide

The title compound was synthesized using precursor II, general procedure B and D using 2-(7-chlorothieno[3,2-b]pyridin-2-yl)-N,N-dimethylethane-1-sulfonamide to give the desired product as the HCl salt. 1H NMR (400 MHz, Methanol-d) δ 8.62 (d, J=5.0 Hz, 1H), 7.52 (d, J=5.0 Hz, 1H), 7.48-7.46 (m, 1H), 7.45 (s, 1H), 7.40-7.39 (m, 1H), 5.75 (dd, J=10.0, 6.9 Hz, 1H), 3.78-3.67 (m, 2H), 3.62 (ddd, J=13.5, 6.0, 3.2 Hz, 1H), 3.57-3.38 (m, 7H), 2.89 (s, 6H), 2.81 (ddd, J=12.8, 6.0, 3.4 Hz, 1H), 2.71 (ddd, J=12.0, 8.1, 4.3 Hz, 2H), 2.56 (ddd, J=12.3, 7.8, 3.3 Hz, 1H). [M+H] 535.0.

### Example 284

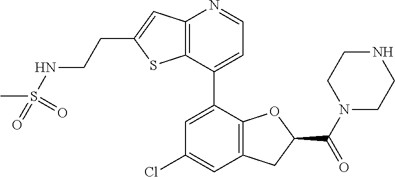 [SMILES: CS(=O)(=O)NCCc1cc2nccc(-c3cc(Cl)cc4c3O[C@@H](C(=O)N3CCNCC3)C4)c2s1]

### (R)-N-(2-(7-(5-chloro-2-(piperazine-1-carbonyl)-2,3-dihydrobenzofuran-7-yl)thieno[3,2-b]pyridin-2-yl)ethyl)methanesulfonamide

The title compound was synthesized using precursor II, general procedure B using 2-(2-(7-chlorothieno[3,2-b]pyridin-2-yl)ethyl)isoindoline-1,3-dione. The phthalimide protecting group was removed with hydrazine, and treatment with methanesulfonylchloride followed by procedure D to give the desired product as the HCl salt. 1H NMR (400 MHz, Methanol-d4) δ 8.99-8.76 (m, 1H), 8.03 (s, 1H), 7.73-7.56 (m, 2H), 7.51 (s, 1H), 5.99-5.82 (m, 1H), 4.00-3.09 (m, 14H), 2.93 (s, 3H). [M+H] 521.0.

### Example 285

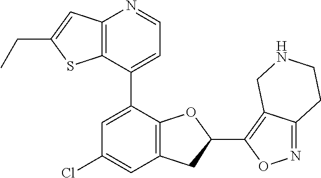 [SMILES: CCc1cc2nccc(-c3cc(Cl)cc4c3O[C@@H](c3onc5c3CNCC5)C4)c2s1]

### (R)-3-(5-chloro-7-(2-ethylthieno[3,2-b]pyridin-7-yl)-2,3-dihydrobenzofuran-2-yl)-4,5,6,7-tetrahydroisoxazolo[4,3-c]pyridine

The title compound was synthesized using precursor III, general procedure C, E and F using 2-ethyl-7-(4,4,5,5-tetramethyl-1,3,2-dioxaborolan-2-yl)thieno[3,2-b]pyridine to give the desired product. 1H NMR (400 MHz, Methanol-d) δ 8.58 (d, J=5.0 Hz, 1H), 7.51 (d, J=2.3 Hz, 1H), 7.45 (d, J=2.1 Hz, 1H), 7.41 (d, J=5.0 Hz, 1H), 7.28 (s, 1H), 6.09 (dd, J=9.9, 6.2 Hz, 1H), 3.81 (dd, J=16.4, 9.9 Hz, 1H), 3.61 (s, 2H), 3.54 (dd, J=16.4, 6.2 Hz, 1H), 3.06-2.98 (m, 2H), 2.95 (t, J=6.0 Hz, 2H), 2.73 (t, J=6.0 Hz, 2H), 1.40 (t, J=7.5 Hz, 3H). [M+H] 438.0.

### Example 286

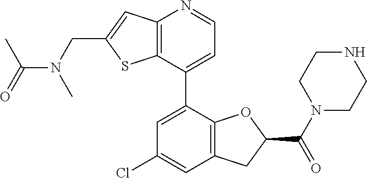 [SMILES: CC(=O)N(C)Cc1cc2nccc(-c3cc(Cl)cc4c3O[C@@H](C(=O)N3CCNCC3)C4)c2s1]

### (R)-N-((7-(5-chloro-2-(piperazine-1-carbonyl)-2,3-dihydrobenzofuran-7-yl)thieno[3,2-b]pyridin-2-yl)methyl)-N-methylacetamide

The title compound was synthesized using precursor II, general procedure B and E using N-((7-chlorothieno[3,2-b]pyridin-2-yl)methyl)-N-methylacetamide to give the desired product as the TFA salt. 1H NMR (400 MHz, CD3OD): δ ppm 7.95 (bs, 1H), 779 (s, 1H), 7.61 (s, 1H), 7.49 (s, 1H), 7.45 (s, 1H), 5.80 (dd, J=9.6, 6.4 Hz, 1H), 4.97 (s, 2H), 3.97-3.14 (m, 10H), 3.08 (s, 3H), 2.11 (s, 3H). [M+H] 485.0.

### Example 287

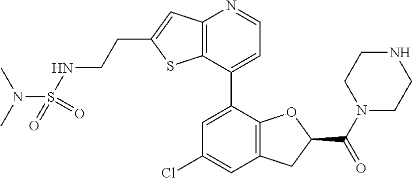 [SMILES: CN(C)S(=O)(=O)NCCc1cc2nccc(-c3cc(Cl)cc4c3O[C@@H](C(=O)N3CCNCC3)C4)c2s1]

### (R)-N-(2-(7-(5-chloro-2-(piperazine-1-carbonyl)-2,3-dihydrobenzofuran-7-yl)thieno[3,2-b]pyridin-2-yl)ethyl)dimethylamimosulfonylurea

The intermediate from Example 284 was treated with N,N-dimethylsulfamoyl chloride followed by procedure D to give the desired product as the HCl salt. [M+H] 550.0.

### Example 288

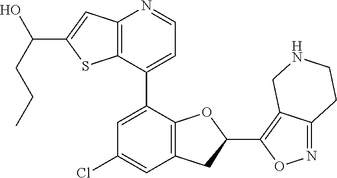 [SMILES: CCCC(O)c1cc2nccc(-c3cc(Cl)cc4c3O[C@@H](c3onc5c3CNCC5)C4)c2s1]

### 1-(7-((R)-5-chloro-2-(4,5,6,7-tetrahydroisoxazolo[4,3-c]pyridin-3-yl)-2,3-dihydrobenzofuran-7-yl)thieno[3,2-b]pyridin-2-yl)butan-1-ol

The title compound was synthesized using precursor III, general procedure C, E and F using 1-(7-(4,4,5,5-tetramethyl-1,3,2-dioxaborolan-2-yl)thieno[3,2-b]pyridin-2-yl)butan-1-ol to give the desired product. 1H NMR (400 MHz, Methanol-d) δ 8.61 (d, J=5.0 Hz, 1H), 7.51 (t, J=2.4 Hz, 1H), 7.49-7.43 (m, 1H), 7.43 (dd, J=5.0, 1.7 Hz, 1H), 7.38 (d, J=1.0 Hz, 1H), 6.09 (dd, J=9.9, 6.2 Hz, 1H), 5.01 (q, J=6.1 Hz, 1H), 3.81 (dd, J=16.4, 9.9 Hz, 1H), 3.62 (d, J=8.1 Hz, 2H), 3.59-3.50 (m, 1H), 3.01-2.92 (m, 2H), 2.74 (t, J=6.0 Hz, 2H), 1.89-1.78 (m, 2H), 1.58-1.37 (m, 2H), 0.98 (t, J=7.4 Hz, 3H). [M+H] 482.0.

### Example 289

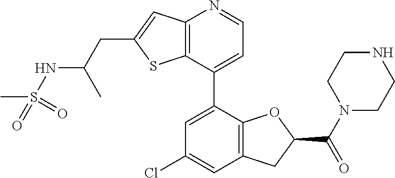 [SMILES: CC(Cc1cc2nccc(-c3cc(Cl)cc4c3O[C@@H](C(=O)N3CCNCC3)C4)c2s1)NS(C)(=O)=O]

### N-(1-(7-((R)-5-chloro-2-(piperazine-1-carbonyl)-2,3-dihydrobenzofuran-7-yl)thieno[3,2-b]pyridin-2-yl)propan-2-yl)methanesulfonamide

The title compound was synthesized using precursor II, general procedure B using 2-(1-(7-chlorothieno[3,2-b]pyridin-2-yl)propan-2-yl)isoindoline-1,3-dione. The phthalimide protecting group was removed with hydrazine, and treatment with methanesulfonylchloride followed by procedure D to give the desired product as the HCl salt.

### Example 290

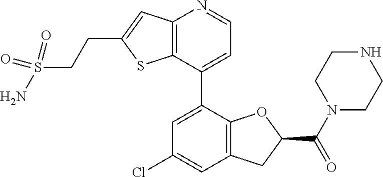 [SMILES: NS(=O)(=O)CCc1cc2nccc(-c3cc(Cl)cc4c3O[C@@H](C(=O)N3CCNCC3)C4)c2s1]

### (R)-2-(7-(5-chloro-2-(piperazine-1-carbonyl)-2,3-dihydrobenzofuran-7-yl)thieno[3,2-b]pyridin-2-yl)ethane-1-sulfonamide

The title compound was synthesized using precursor II, general procedure B and E using 2-(7-chlorothieno[3,2-b]pyridin-2-yl)ethane-1-sulfonamide to give the desired product as the TFA salt. [M+H] 507.0.

### Example 291

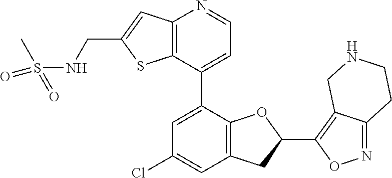 [SMILES: CS(=O)(=O)NCc1cc2nccc(-c3cc(Cl)cc4c3O[C@@H](c3onc5c3CNCC5)C4)c2s1]

### (R)-N-((7-(5-chloro-2-(4,5,6,7-tetrahydroisoxazolo[4,3-c]pyridin-3-yl)-2,3-dihydrobenzofuran-7-yl)thieno[3,2-b]pyridin-2-yl)methyl)methanesulfonamide

The title compound was synthesized using precursor II, general procedure G and D using N-((7-chlorothieno[3,2-b]pyridin-2-yl)methyl)methanesulfonamide to give the desired product as the HCl salt. ¹H NMR (400 MHz, CD3OD): δ ppm 8.05 (bs, 1H), 7.57 (s, 1H), 7.54 (s, 1H), 7.14 (s, 1H), 7.12 (s, 1H), 6.00 (bs, 1H), 4.68-4.66 (m 2H), 4.13 (bs, 2H), 3.89-3.48 (m, 4H), 3.10 (bs, 2H), 3.02 (s, 3H). [M+H] 517.0.

### Example 292

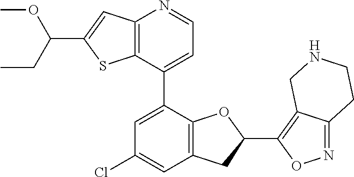 [SMILES: CCC(OC)c1cc2nccc(-c3cc(Cl)cc4c3O[C@@H](c3onc5c3CNCC5)C4)c2s1]

### 3-((2R)-5-chloro-7-(2-(1-methoxypropyl)thieno[3,2-b]pyridin-7-yl)-2,3-dihydrobenzofuran-2-yl)-4,5,6,7-tetrahydroisoxazolo[4,3-c]pyridine

The intermediate from Example 203 was treated with sodium hydride and methyl iodide, then general procedure E and F to give the desired product. ¹H NMR (400 MHz, Methanol-d) δ 8.63 (d, J=5.0 Hz, 1H), 7.52-7.50 (m, 1H), 7.48-7.45 (m, 2H), 7.45 (d, J=1.6 Hz, 1H), 6.08 (ddd, J=10.0, 6.2, 2.0 Hz, 1H), 4.52 (t, J=6.2 Hz, 1H), 3.81 (dd, J=16.4, 10.0 Hz, 1H), 3.64 (s, 2H), 3.56 (dd, J=16.4, 6.2 Hz, 1H), 3.33 (d, J=5.5 Hz, 3H), 2.96 (t, J=6.0 Hz, 2H), 2.79-2.67 (m, 2H), 1.98-1.85 (m, 1H), 1.85-1.71 (m, 1H), 0.95 (td, J=7.4, 4.3 Hz, 3H). [M+H] 482.0.

### Example 293

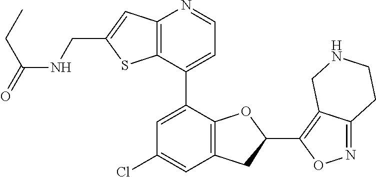 [SMILES: CCC(=O)NCc1cc2nccc(-c3cc(Cl)cc4c3O[C@@H](c3onc5c3CNCC5)C4)c2s1]

### (R)-N-((7-(5-chloro-2-(4,5,6,7-tetrahydroisoxazolo[4,3-c]pyridin-3-yl)-2,3-dihydrobenzofuran-7-yl)thieno[3,2-b]pyridin-2-yl)methyl)propionamide

The title compound was synthesized using precursor III, general procedure G and D using N-((7-chlorothieno[3,2-b]pyridin-2-yl)methyl)propionamide to give the desired product as the HCl salt. [M+H] 495.0.

### Example 294

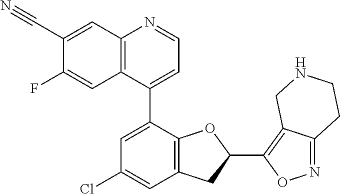 [SMILES: N#Cc1cc2nccc(-c3cc(Cl)cc4c3O[C@@H](c3onc5c3CNCC5)C4)c2cc1F]

### (R)-4-(5-chloro-2-(4,5,6,7-tetrahydroisoxazolo[4,3-c]pyridin-3-yl)-2,3-dihydrobenzofuran-7-yl)-6-fluoroquinoline-7-carbonitrile

The title compound was synthesized using precursor III, general procedure G and E using 4-chloro-6-fluoroquinoline-7-carbonitrile to give the desired product as the TFA salt. ¹H NMR (400 MHz, CD3OD): δ ppm 9.04 (d, J=4.4 Hz, 1H), 8.60 (d, J=6.3 Hz, 1H), 7.67 (d, J=4.4 Hz, 1H), 7.59 (d, J=10.7 Hz, 1H), 7.54-7.47 (m, 1H), 7.36 (d, J=2.2 Hz, 1H), 6.20-6.01 (m, 1H), 4.13-3.96 (m, 2H), 3.88 (dd, J=16.8, 9.6 Hz, 1H), 3.58 (dd, J=16.4, 6.3 Hz, 1H), 3.45 (t, J=6.5 Hz, 2H), 3.07 (t, J=6.5 Hz, 2H).

### Example 295

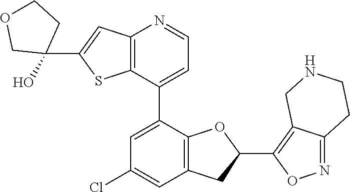 [SMILES: O[C@@]1(c2cc3nccc(-c4cc(Cl)cc5c4O[C@@H](c4onc6c4CNCC6)C5)c3s2)CCOC1]

### (S)-3-(7-((R)-5-chloro-2-(4,5,6,7-tetrahydroisoxazolo[4,3-c]pyridin-3-yl)-2,3-dihydrobenzofuran-7-yl)thieno[3,2-b]pyridin-2-yl)tetrahydrofuran-3-ol

The title compound was synthesized using precursor III, general procedure C and E using 3-(7-(4,4,5,5-tetramethyl-1,3,2-dioxaborolan-2-yl)thieno[3,2-b]pyridin-2-yl)tetrahydrofuran-3-ol to give the desired product as the TFA salt. The pure isomer was obtained after chromatography. ¹H NMR (400 MHz, Methanol-d4) δ 8.63 (d, J=5.0 Hz, 1H), 7.51-7.49 (m, 1H), 7.48 (s, 1H), 7.48-7.46 (m, 1H), 7.44 (d, J=5.0 Hz, 1H), 6.10 (dd, J=9.9, 6.0 Hz, 1H), 4.60 (s, 2H), 4.19-4.11 (m, 1H), 3.82 (dd, J=16.3, 10.0 Hz, 2H), 3.69 (s, 2H), 3.56 (dd, J=16.2, 6.0 Hz, 1H), 3.07-2.97 (m, 2H), 2.79 (t, J=6.1 Hz, 2H), 2.51 (dt, J=12.7, 9.0 Hz, 1H), 2.41-2.31 (m, 1H). [M+H] 496.0.

### Example 296

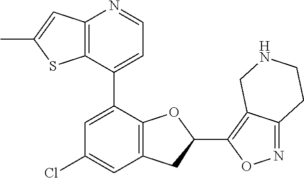 [SMILES: Cc1cc2nccc(-c3cc(Cl)cc4c3O[C@@H](c3onc5c3CNCC5)C4)c2s1]

### (R)-3-(5-chloro-7-(2-methylthieno[3,2-b]pyridin-7-yl)-2,3-dihydrobenzofuran-2-yl)-4,5,6,7-tetrahydroisoxazolo[4,3-c]pyridine

The title compound was synthesized using precursor III, general procedure C and D using 2-methyl-7-(4,4,5,5-tetramethyl-1,3,2-dioxaborolan-2-yl)thieno[3,2-b]pyridine to give the desired product as the HCl salt. ¹H NMR (400 MHz, Methanol-d4) δ 8.75 (d, J=5.8 Hz, 1H), 7.81 (d, J=5.8 Hz, 1H), 7.65 (d, J=2.1 Hz, 1H), 7.56 (d, J=2.1 Hz, 1H), 7.48 (d, J=1.6 Hz, 1H), 6.22 (dd, J=9.9, 6.8 Hz, 1H), 4.20-4.02 (m, 2H), 3.87 (dd, J=16.5, 9.9 Hz, 1H), 3.58 (dd, J=16.5, 6.8 Hz, 1H), 3.45 (t, J=6.4 Hz, 2H), 3.07 (t, J=6.4 Hz, 2H), 2.76 (s, 3H). [M+H] 424.0.

### Example 297

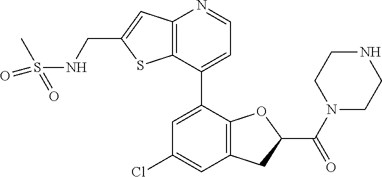 [SMILES: CS(=O)(=O)NCc1cc2nccc(-c3cc(Cl)cc4c3O[C@@H](C(=O)N3CCNCC3)C4)c2s1]

### (R)-N-((7-(5-chloro-2-(piperazine-1-carbonyl)-2,3-dihydrobenzofuran-7-yl)thieno[3,2-b]pyridin-2-yl)methyl)methanesulfonamide

The title compound was synthesized using precursor II, general procedure B and D using N-((7-chlorothieno[3,2-b]pyridin-2-yl)methyl)methanesulfonamide to give the desired product as the HCl salt. ¹H NMR (400 MHz, CD3OD): δ ppm 8.89 (s, 1H), 8.03 (s, 1H), 7.75 (d, J=6.3 Hz, 1H), 7.65 (d, J=25.9 Hz, 1H), 7.55 (s, 1H), 5.90 (s, 1H), 4.76 (s, 2H), 3.83 (d, J=27.0 Hz, 2H), 3.85-3.40 (m, 6H), 3.23 (s, 2H), 3.06 (d, J=11.3 Hz, 3H).

### Example 298

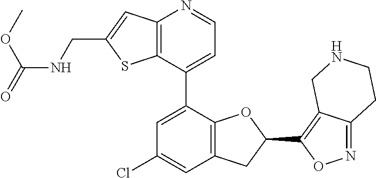 [SMILES: COC(=O)NCc1cc2nccc(-c3cc(Cl)cc4c3O[C@@H](c3onc5c3CNCC5)C4)c2s1]

### Methyl (R)-((7-(5-chloro-2-(4,5,6,7-tetrahydroisoxazolo[4,3-c]pyridin-3-yl)-2,3-dihydrobenzofuran-7-yl)thieno[3,2-b]pyridin-2-yl)methyl)carbamate

The title compound was synthesized using precursor III, general procedure G and D using methyl ((7-chlorothieno[3,2-b]pyridin-2-yl)methyl)carbamate to give the desired product as the HCl salt. [M+H] 497.0.

### Example 299

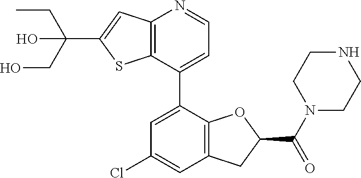 [SMILES: CCC(O)(CO)c1cc2nccc(-c3cc(Cl)cc4c3O[C@@H](C(=O)N3CCNCC3)C4)c2s1]

### ((2R)-5-chloro-7-(2-(1,2-dihydroxybutan-2-yl)thieno[3,2-b]pyridin-7-yl)-2,3-dihydrobenzofuran-2-yl)(piperazin-1-yl)methanone

The title compound was synthesized using precursor II, general procedure B and D using 7-chloro-2-(4-ethyl-2,2-dimethyl-1,3-dioxolan-4-yl)thieno[3,2-b]pyridine to give the desired product as the HCl salt. [M+H] 488.0.

### Example 300

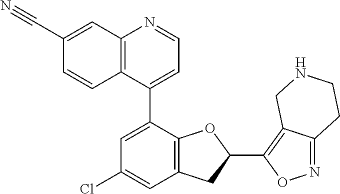 [SMILES: N#Cc1ccc2c(-c3cc(Cl)cc4c3O[C@@H](c3onc5c3CNCC5)C4)ccnc2c1]

### (R)-4-(5-chloro-2-(4,5,6,7-tetrahydroisoxazolo[4,3-c]pyridin-3-yl)-2,3-dihydrobenzofuran-7-yl)quinoline-7-carbonitrile

The title compound was synthesized using precursor III, general procedure C and D using 4-(4,4,5,5-tetramethyl-1,3,2-dioxaborolan-2-yl)quinoline-7-carbonitrile to give the desired product as the HCl salt. 1H NMR (400 MHz, CD3OD): δ ppm 9.44 (d, J=5.3 Hz, 1H), 8.77 (d, J=10.4 Hz, 1H), 8.29 (m, 3H), 7.63 (s, 1H), 7.50 (dd, J=14.4, 9.0 Hz, 1H), 6.20 (dd, J=9.8, 6.4 Hz, 1H), 4.19-4.03 (m, 2H), 3.93 (dd, J=16.6, 9.7 Hz, 1H), 3.85-3.06 (m, 5H).

### Example 301

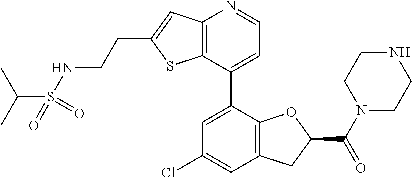 [SMILES: CC(C)S(=O)(=O)NCCc1cc2nccc(-c3cc(Cl)cc4c3O[C@@H](C(=O)N3CCNCC3)C4)c2s1]

### (R)-N-(2-(7-(5-chloro-2-(piperazine-1-carbonyl)-2,3-dihydrobenzofuran-7-yl)thieno[3,2-b]pyridin-2-yl)ethyl)propane-2-sulfonamide

The intermediate from Example 284 was treated with 2-propanesulfonyl chloride followed by procedure D to give the desired product as the HCl salt. ¹H NMR (400 MHz, Methanol-d4) δ 8.86 (d, J=5.8 Hz, 1H), 8.07 (d, J=6.0 Hz, 1H), 7.68 (s, 1H), 7.66 (d, J=2.2 Hz, 1H), 7.54 (d, J=2.0 Hz, 1H), 5.93 (dd, J=9.1, 7.5 Hz, 1H), 3.93-3.08 (m, 15H), 1.32 (d, J=6.8 Hz, 6H). [M+H] 549.0.

### Example 302

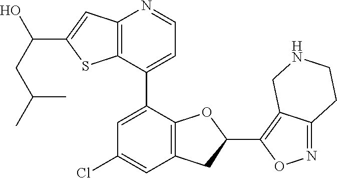 [SMILES: CC(C)CC(O)c1cc2nccc(-c3cc(Cl)cc4c3O[C@@H](c3onc5c3CNCC5)C4)c2s1]

### 1-(7-((R)-5-chloro-2-(4,5,6,7-tetrahydroisoxazolo[4,3-c]pyridin-3-yl)-2,3-dihydrobenzofuran-7-yl)thieno[3,2-b]pyridin-2-yl)-3-methylbutan-1-ol

The title compound was synthesized using precursor III, general procedure C, E and F using 3-methyl-1-(7-(4,4,5,5-tetramethyl-1,3,2-dioxaborolan-2-yl)thieno[3,2-b]pyridin-2-yl)butan-1-ol to give the desired product. ¹H NMR (400 MHz, Chloroform-d) δ 8.67 (t, J=4.6 Hz, 1H), 7.44-7.20 (m, 4H), 6.02-5.85 (m, 1H), 5.17-4.97 (m, 1H), 3.77-3.51 (m, 5H), 3.22-2.49 (m, 4H), 1.91-1.47 (m, 2H), 1.25 (s, 6H). [M+H] 496.0.

### Example 303

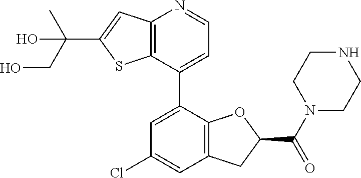 [SMILES: CC(O)(CO)c1cc2nccc(-c3cc(Cl)cc4c3O[C@@H](C(=O)N3CCNCC3)C4)c2s1]

### ((2R)-5-chloro-7-(2-(1,2-dihydroxypropan-2-yl)thieno[3,2-b]pyridin-7-yl)-2,3-dihydrobenzofuran-2-yl)(piperazin-1-yl)methanone

The title compound was synthesized using precursor II, general procedure B and D using 2-(7-chlorothieno[3,2-b]pyridin-2-yl)propane-1,2-diol to give the desired product as the HCl salt. ¹H NMR (400 MHz, CD3OD): δ ppm 8.99 (bs, 1H), 8.12 (bs, 1H), 7.67 (s, 1H), 7.64 (s, 1H), 7.54 (s, 1H), 5.90 (quin, J=5.3, 1H), 3.92-3.12 (m, 13H), 1.69 (s, 3H). [M+H] 474.0.

### Example 304

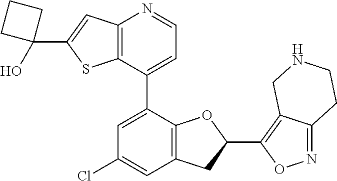 [SMILES: OC1(c2cc3nccc(-c4cc(Cl)cc5c4O[C@@H](c4onc6c4CNCC6)C5)c3s2)CCC1]

### (R)-1-(7-(5-chloro-2-(4,5,6,7-tetrahydroisoxazolo[4,3-c]pyridin-3-yl)-2,3-dihydrobenzofuran-7-yl)thieno[3,2-b]pyridin-2-yl)cyclobutan-1-ol

The title compound was synthesized using precursor III, general procedure C and D using 1-(7-(4,4,5,5-tetramethyl-1,3,2-dioxaborolan-2-yl)thieno[3,2-b]pyridin-2-yl)cyclobutan-1-ol to give the product as the HCl salt. ¹H NMR (400 MHz, CD3OD): δ ppm 8.60 (d, J=4.8 Hz, 1H), 7.49 (s, 2H), 7.44 (s, 1H), 7.41 (d, J=4.8 Hz, 1H), 6.06 (dd, J=10.0, 6.4 Hz, 1H), 3.82-3.51 (m, 3H), 2.93 (t, J=6.0 Hz, 2H), 2.72 (t, J=6.0 Hz, 2H), 2.58-2.43 (m, 4H), 2.03-1.84 (m, 2H). [M+H] 480.0.

### Example 305

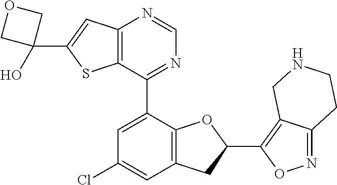 [SMILES: OC1(c2cc3ncnc(-c4cc(Cl)cc5c4O[C@@H](c4onc6c4CNCC6)C5)c3s2)COC1]

### (R)-3-(4-(5-chloro-2-(4,5,6,7-tetrahydroisoxazolo[4,3-c]pyridin-3-yl)-2,3-dihydrobenzofuran-7-yl)thieno[3,2-d]pyrimidin-6-yl)oxetan-3-ol

The title compound was synthesized using precursor III, general procedure G and D using 3-(4-chlorothieno[3,2-d]pyrimidin-6-yl)oxetan-3-ol to give the desired product as the HCl salt. ¹H NMR (400 MHz, CD3OD): δ ppm 9.15 (s, 1H), 7.72 (s, 1H), 7.51 (s, 1H), 7.44 (s, 1H), 6.13 (dd, J=10.0, 5.6 Hz, 1H), 4.93-4.80 (m, 4H), 3.83-3.57 (m, 5H), 2.99-2.95 (m, 2H), 2.74 (t, J=5.6 Hz, 2H). [M+H] 483.0.

### Example 306

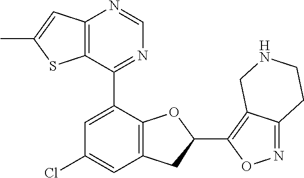 [SMILES: Cc1cc2ncnc(-c3cc(Cl)cc4c3O[C@@H](c3onc5c3CNCC5)C4)c2s1]

### (R)-3-(5-chloro-7-(6-methylthieno[3,2-d] pyrimidin-4-yl)-2,3-dihydrobenzofuran-2-yl)-4,5,6,7-tetrahydroisoxazolo[4,3-c]pyridine

The title compound was synthesized using precursor III, general procedure G and D using 4-chloro-6-methylthieno[3,2-d]pyrimidine to give the desired product as the HCl salt. ¹H NMR (400 MHz, CD3OD) δ 9.37 (s, 1H), 7.78 (d, J=2.2 Hz, 1H), 7.69-7.65 (m, 1H), 7.56 (d, J=1.1 Hz, 1H), 6.33 (dd, J=10.0, 6.7 Hz, 1H), 4.30 (s, 2H), 3.91 (dd, J=16.5, 10.0 Hz, 1H), 3.76-3.71 (m, 1H), 3.63-3.60 (m, 1H), 3.59-3.55 (m, 1H), 3.51 (t, J=6.7 Hz, 2H), 2.85 (d, J=1.1 Hz, 3H). [M+H] 425.1.

### Example 307

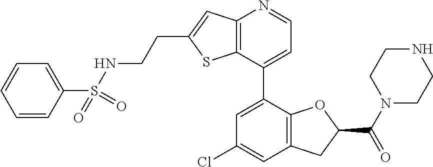 [SMILES: O=C([C@H]1Cc2cc(Cl)cc(-c3ccnc4cc(CCNS(=O)(=O)c5ccccc5)sc34)c2O1)N1CCNCC1]

### (R)-N-(2-(7-(5-chloro-2-(piperazine-1-carbonyl)-2,3-dihydrobenzofuran-7-yl)thieno[3,2-b]pyridin-2-yl)ethyl)benzenesulfonamide

The intermediate from Example 284 was treated with benzenesulfonyl chloride followed by procedure D to give the desired product as the HCl salt. ¹H NMR (400 MHz, Methanol-d4) δ 8.88-8.81 (m, 1H), 8.12-8.01 (m, 1H), 7.82-7.76 (m, 2H), 7.63 (s, 1H), 7.59-7.44 (m, 5H), 5.95 (q, J=7.8 Hz, 1H), 3.94-3.12 (m, 14H). [M+H] 583.0.

### Example 308

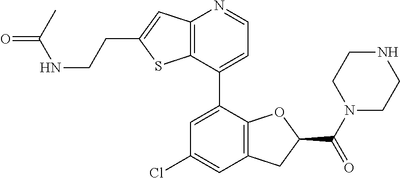 [SMILES: CC(=O)NCCc1cc2nccc(-c3cc(Cl)cc4c3O[C@@H](C(=O)N3CCNCC3)C4)c2s1]

### (R)-N-(2-(7-(5-chloro-2-(piperazine-1-carbonyl)-2,3-dihydrobenzofuran-7-yl)thieno[3,2-b]pyridin-2-yl)ethyl)acetamide

The intermediate from Example 284 was treated with acetic anhydride followed by procedure D to give the desired product as the HCl salt. ¹H NMR (400 MHz, Methanol-d4) δ 9.03-8.76 (m, 1H), 8.13 (s, 1H), 7.76-7.59 (m, 2H), 7.54 (s, 1H), 6.11-5.78 (m, 1H), 4.01-3.05 (m, 14H), 1.94 (s, 3H). [M+H] 485.0.

### Example 309

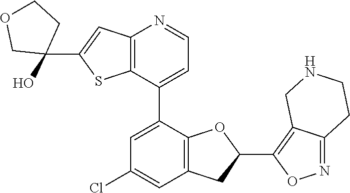 [SMILES: O[C@]1(c2cc3nccc(-c4cc(Cl)cc5c4O[C@@H](c4onc6c4CNCC6)C5)c3s2)CCOC1]

### (R)-3-(7-((R)-5-chloro-2-(4,5,6,7-tetrahydroisoxazolo[4,3-c]pyridin-3-yl)-2,3-dihydrobenzofuran-7-yl)thieno[3,2-b]pyridin-2-yl)tetrahydrofuran-3-ol

Other isomer of Example 295 obtained by chromatography. ¹H NMR (400 MHz, Methanol-d4) δ 8.62 (d, J=5.0 Hz, 1H), 7.52-7.49 (m, 1H), 7.48 (s, 1H), 7.47-7.45 (m, 1H), 7.44 (d, J=5.0 Hz, 1H), 6.10 (dd, J=9.9, 6.1 Hz, 1H), 4.60 (s, 2H), 4.19-4.10 (m, 2H), 3.82 (dd, J=16.4, 9.8 Hz, 1H), 3.67 (s, 2H), 3.56 (dd, J=16.4, 6.0 Hz, 1H), 3.03 (t, J=7.3 Hz, 2H), 2.78 (t, J=5.9 Hz, 2H), 2.49 (td, J=13.0, 8.9 Hz, 1H), 2.36 (ddd, J=13.0, 6.5, 4.2 Hz, 1H). [M+H] 496.0.

### Example 310

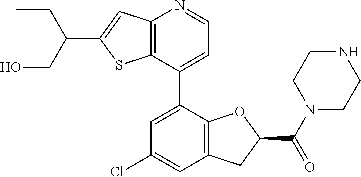 [SMILES: CCC(CO)c1cc2nccc(-c3cc(Cl)cc4c3O[C@@H](C(=O)N3CCNCC3)C4)c2s1]

### ((2R)-5-chloro-7-(2-(1-hydroxybutan-2-yl)thieno[3,2-b]pyridin-7-yl)-2,3-dihydrobenzofuran-2-yl)(piperazin-1-yl)methanone

The title compound was synthesized using precursor II, general procedure B and D using 2-(7-chlorothieno[3,2-b]pyridin-2-yl)butan-1-ol to give the desired product as the HCl salt. ¹H NMR (400 MHz, CD3OD): δ ppm 8.89 (s, 1H), 8.13 (s, 1H), 7.68 (d, J=5.1 Hz, 2H), 7.54 (s, 1H), 5.94 (s, 1H), 4.02-3.77 (m, 6H), 3.58 (dt, J=6.0, 4.2 Hz, 2H), 3.41-3.10 (m, 4H), 1.98 (d, J=23.5 Hz, 1H), 1.74 (dd, J=41.7, 7.2 Hz, 1H), 1.23-1.13 (m, 1H), 0.97 (dd, J=15.7, 8.5 Hz, 3H).

### Example 311

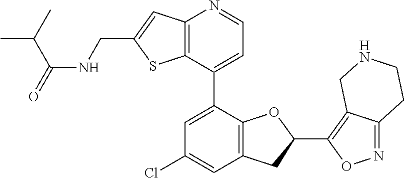 [SMILES: CC(C)C(=O)NCc1cc2nccc(-c3cc(Cl)cc4c3O[C@@H](c3onc5c3CNCC5)C4)c2s1]

### (R)-N-((7-(5-chloro-2-(4,5,6,7-tetrahydroisoxazolo[4,3-c]pyridin-3-yl)-2,3-dihydrobenzofuran-7-yl)thieno[3,2-b]pyridin-2-yl)methyl)isobutyramide

The title compound was synthesized using precursor III, general procedure G and D using N-((7-chlorothieno[3,2-b]pyridin-2-yl)methyl)isobutyramide to give the desired product as the HCl salt. ¹H NMR (400 MHz, CD3OD): δ ppm 8.88 (bs, 1H), 8.02 (s, 1H), 7.71 (s, 1H), 7.62 (s, 1H), 6.25 (bs, 1H), 4.95 (s, 2H), 4.19 (bs, 1H), 4.06-3.13 (m, 4H), 2.52 (hept, J=6.8 Hz, 1H), 1.09 (d, J=6.8 Hz, 6H). [M+H] 509.0.

### Example 312

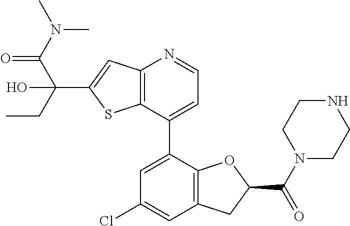 [SMILES: CCC(O)(C(=O)N(C)C)c1cc2nccc(-c3cc(Cl)cc4c3O[C@@H](C(=O)N3CCNCC3)C4)c2s1]

### 2-(7-((R)-5-chloro-2-(piperazine-1-carbonyl)-2,3-dihydrobenzofuran-7-yl)thieno[3,2-b]pyridin-2-yl)-2-hydroxy-N,N-dimethylbutanamide

The title compound was synthesized using precursor II, general procedure B and D using 2-(7-chlorothieno[3,2-b]pyridin-2-yl)-2-hydroxy-N,N-dimethylbutanamide to give the desired product as the HCl salt. [M+H] 529.1.

### Example 313

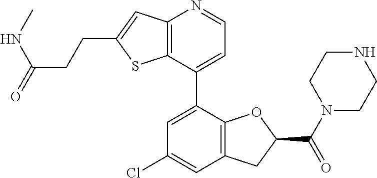 [SMILES: CNC(=O)CCc1cc2nccc(-c3cc(Cl)cc4c3O[C@@H](C(=O)N3CCNCC3)C4)c2s1]

### (R)-3-(7-(5-chloro-2-(piperazine-1-carbonyl)-2,3-dihydrobenzofuran-7-yl)thieno[3,2-b]pyridin-2-yl)-N-methylpropanamide

The title compound was synthesized using precursor II, general procedure B using ethyl 3-(7-methylthieno[3,2-b]pyridin-2-yl)propanoate. The ester was hydrolyzed with lithium hydroxide, coupled to methylamine using HATU, and general procedure D to give the desired compound as the HCl salt. [M+H] 485.0.

### Example 314

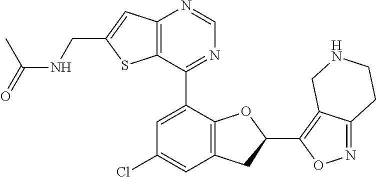 [SMILES: CC(=O)NCc1cc2ncnc(-c3cc(Cl)cc4c3O[C@@H](c3onc5c3CNCC5)C4)c2s1]

### (R)-N-((4-(5-chloro-2-(4,5,6,7-tetrahydroisoxazolo[4,3-c]pyridin-3-yl)-2,3-dihydrobenzofuran-7-yl)thieno[3,2-d]pyrimidin-6-yl)methyl)acetamide

The title compound was synthesized using precursor III, general procedure C using 2-((4-chlorothieno[3,2-d]pyrimidin-6-yl)methyl)isoindoline-1,3-dione. The phthalimide was deprotected with hydrazine, acylated with acetic anhydride, and general procedure D to give the desired product as the HCl salt. ¹H NMR (400 MHz, CD3OD): δ ppm 9.18 (s, 1H), 7.64-7.60 (m, 1H), 7.56 (s, 1H), 7.47 (s, 1H), 6.26-6.17 (m, 1H), 4.81-4.74 (m, 1H), 4.71 (s, 1H), 4.28 (s, 2H), 3.92-3.81 (m, 2H), 3.78-3.71 (m, 2H), 3.13 (s, 2H), 2.02 (s, 3H). [M+H] 483.0.

### Example 315

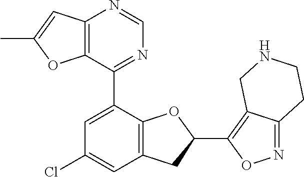 [SMILES: Cc1cc2ncnc(-c3cc(Cl)cc4c3O[C@@H](c3onc5c3CNCC5)C4)c2o1]

### (R)-3-(5-chloro-7-(6-methylfuro[3,2-d]pyrimidin-4-yl)-2,3-dihydrobenzofuran-2-yl)-4,5,6,7-tetrahydroisoxazolo[4,3-c]pyridine

The title compound was synthesized using precursor III, general procedure G and D using 4-chloro-6-methylfuro[3,2-d]pyrimidine to give the desired product as the HCl salt. ¹H NMR (400 MHz, CD3OD) δ 8.99 (s, 1H), 7.88-7.68 (m, 1H), 7.57-7.38 (m, 1H), 6.83 (d, J=1.0 Hz, 1H), 6.15 (dd, J=10.0, 6.2 Hz, 1H), 4.21 (d, J=5.1 Hz, 2H), 3.77 (dd, J=16.5, 10.1 Hz, 1H), 3.51-3.41 (m, 2H), 3.38 (t, J=6.4 Hz, 2H), 2.99 (t, J=6.4 Hz, 2H), 2.55 (d, J=1.0 Hz, 2H). [M+H] 409.1.

### Example 316

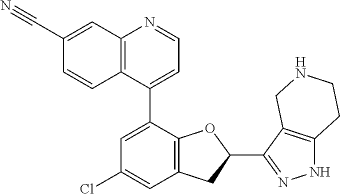 [SMILES: N#Cc1ccc2c(-c3cc(Cl)cc4c3O[C@@H](c3n[nH]c5c3CNCC5)C4)ccnc2c1]

### (R)-4-(5-chloro-2-(4,5,6,7-tetrahydro-1H-pyrazolo[4,3-c]pyridin-3-yl)-2,3-dihydrobenzofuran-7-yl)quinoline-7-carbonitrile

The title compound was synthesized using precursor IV, general procedure C and D using 4-(4,4,5,5-tetramethyl-1,3,2-dioxaborolan-2-yl)quinoline-7-carbonitrile to give the desired product as the HCl salt. ¹H NMR (400 MHz, CD3OD) δ 9.40 (d, J=5.6 Hz, 1H), 8.70 (d, J=1.0 Hz, 1H), 8.30 (d, J=8.9 Hz, 1H), 8.22 (d, J=5.5 Hz, 1H), 8.10 (d, J=10.0 Hz, 1H), 7.60 (d, J=2.1 Hz, 1H), 7.46 (d, J=2.1 Hz, 1H), 6.08-5.94 (m, 1H), 3.99 (dt, J=26.1, 12.9 Hz, 1H), 3.85-3.76 (m, 1H), 3.76-3.54 (m, 4H), 3.01 (t, J=6.0 Hz, 2H).

### Example 317

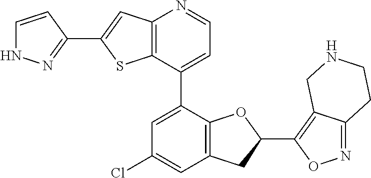 [SMILES: Clc1cc2c(c(-c3ccnc4cc(-c5cc[nH]n5)sc34)c1)O[C@@H](c1onc3c1CNCC3)C2]

### (R)-3-(7-(2-(1H-pyrazol-3-yl)thieno[3,2-b]pyridin-7-yl)-5-chloro-2,3-dihydrobenzofuran-2-yl)-4,5,6,7-tetrahydroisoxazolo[4,3-c]pyridine

The title compound was synthesized using precursor III, general procedure C and E using 2-(1H-pyrazol-3-yl)-7-(4,4,5,5-tetramethyl-1,3,2-dioxaborolan-2-yl)thieno[3,2-b]pyridine to give the desired product as the TFA salt. ¹H NMR (400 MHz, DMSO-d6) δ 9.06-8.84 (m, 2H), 8.72-8.67 (m, 1H), 7.95-7.92 (m, 1H), 7.91-7.86 (m, 1H), 7.63-7.57 (m, 2H), 7.43-7.38 (m, 1H), 6.96-6.92 (m, 1H), 6.33-6.24 (m, 1H), 4.21-4.07 (m, 2H), 3.49-3.29 (m, 4H), 3.08-2.91 (m, 2H). [M+H] 476.0.

### Example 318

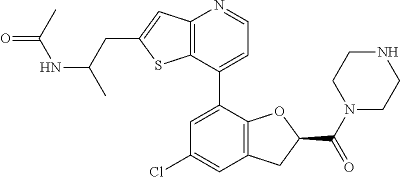 [SMILES: CC(=O)NC(C)Cc1cc2nccc(-c3cc(Cl)cc4c3O[C@@H](C(=O)N3CCNCC3)C4)c2s1]

### N-(1-(7-((R)-5-chloro-2-(piperazine-1-carbonyl)-2,3-dihydrobenzofuran-7-yl)thieno[3,2-b]pyridin-2-yl)propan-2-yl)acetamide

The title compound was synthesized by treatment of the intermediate from Example 289 with acetic anhydride followed by procedure D to give the desired product as the HCl salt.

### Example 319

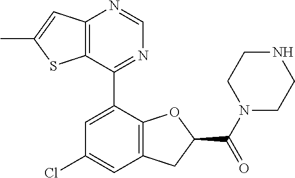 [SMILES: Cc1cc2ncnc(-c3cc(Cl)cc4c3O[C@@H](C(=O)N3CCNCC3)C4)c2s1]

### (R)-(5-chloro-7-(6-methylthieno[3,2-d]pyrimidin-4-yl)-2,3-dihydrobenzofuran-2-yl)(piperazin-1-yl)methanone

The title compound was synthesized using precursor II, general procedure B and D using 4-chloro-6-methylthieno[3,2-d]pyrimidine to give the desired product as the HCl salt. 1H NMR (400 MHz, CD3OD) δ 7.68-7.60 (m, 1H), 7.58-7.53 (m, 1H), 7.51 (s, 1H), 7.40 (s, 1H), 5.85 (dd, J=10.1, 7.0 Hz, 1H), 3.98 (s, 2H), 3.86 (dd, J=16.5, 7.0 Hz, 2H), 3.67 (s, 1H), 3.54 (dd, J=16.5, 9.8 Hz, 2H), 3.21 (d, J=20.9 Hz, 2H), 3.05 (s, 1H), 2.77 (s, 3H). [M+H] 415.1.

### Example 320

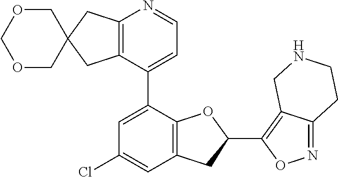 [SMILES: Clc1cc2c(c(-c3ccnc4c3CC3(COCOC3)C4)c1)O[C@@H](c1onc3c1CNCC3)C2]

### (R)-4-(5-chloro-2-(4,5,6,7-tetrahydroisoxazolo[4,3-c]pyridin-3-yl)-2,3-dihydrobenzofuran-7-yl)-5,7-dihydrospiro[cyclopenta[b]pyridine-6,5′-[1,3]dioxane]

The title compound was synthesized using precursor III, general procedure C and D using 4-(4,4,5,5-tetramethyl-1,3,2-dioxaborolan-2-yl)-5,7-dihydrospiro[cyclopenta[b]pyridine-6,5′-[1,3]dioxane] to give the desired product as the HCl salt. 1H NMR (400 MHz, Methanol-d4) δ 8.47 (d, J=5.8 Hz, 1H), 7.57 (d, J=5.7 Hz, 1H), 7.50-7.45 (m, 1H), 7.36 (d, J=2.2 Hz, 1H), 6.19 (dd, J=9.9, 6.5 Hz, 1H), 4.85 (s, 2H), 4.19 (s, 2H), 3.89-3.64 (m, 5H), 3.62-3.45 (m, 3H), 3.16-3.08 (m, 4H), 3.08-2.83 (m, 2H). [M+H] 466.0.

### Example 321

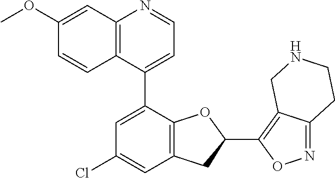 [SMILES: COc1ccc2c(-c3cc(Cl)cc4c3O[C@@H](c3onc5c3CNCC5)C4)ccnc2c1]

### (R)-3-(5-chloro-7-(7-methoxyquinolin-4-yl)-2,3-dihydrobenzofuran-2-yl)-4,5,6,7-tetrahydroisoxazolo[4,3-c]pyridine

The title compound was synthesized using precursor III, general procedure C and D using 7-methoxy-4-(4,4,5,5-tetramethyl-1,3,2-dioxaborolan-2-yl)quinoline to give the desired product as the HCl salt. 1H NMR (400 MHz, CD3OD): δ ppm 9.05 (d, J=5.7 Hz, 1H), 8.21 (d, J=9.4 Hz, 1H), 8.03 (d, J=5.7 Hz, 1H), 7.83 (dd, J=9.5, 2.6 Hz, 1H), 7.69-7.56 (m, 1H), 7.50 (d, J=2.2 Hz, 1H), 7.28 (d, J=2.7 Hz, 1H), 6.16 (s, 1H), 4.25-3.96 (m, 2H), 3.92 (s, 3H), 3.81-3.69 (m, 1H), 3.66-3.40 (m, 3H), 3.10 (t, J=6.4 Hz, 2H). [M+H] 434.0.

### Example 322

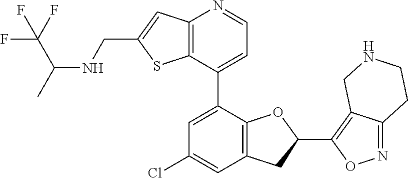 [SMILES: CC(NCc1cc2nccc(-c3cc(Cl)cc4c3O[C@@H](c3onc5c3CNCC5)C4)c2s1)C(F)(F)F]

### N-((7-((R)-5-chloro-2-(4,5,6,7-tetrahydroisoxazolo[4,3-c]pyridin-3-yl)-2,3-dihydrobenzofuran-7-yl)thieno[3,2-b]pyridin-2-yl)methyl)-1,1,1-trifluoropropan-2-amine

The title compound was synthesized using precursor III, general procedure G and D using N-((7-chlorothieno[3,2-b]pyridin-2-yl)methyl)-1,1,1-trifluoropropan-2-amine to give the desired product as the HCl salt. 1H NMR (400 MHz, CD3OD): δ ppm 8.96 (bs, 1H), 8.07 (bs, 1H), 7.95 (s, 1H), 7.74 (s, 1H), 7.63 (s, 1H), 6.28 (bs, 1H), 4.79 (q, J=8.4 Hz, 2H), 4.19-4.16 (m, 2H), 3.95-3.88 (m, 2H), 3.71-3.31 (m, 4H), 3.11 (t, J=5.2 Hz, 2H, 1.52 (d, J=5.4 Hz, 3H). [M+H] 535.0.

### Example 323

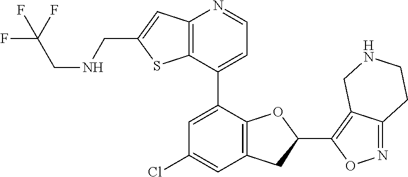 [SMILES: FC(F)(F)CNCc1cc2nccc(-c3cc(Cl)cc4c3O[C@@H](c3onc5c3CNCC5)C4)c2s1]

### (R)-N-((7-(5-chloro-2-(4,5,6,7-tetrahydroisoxazolo[4,3-c]pyridin-3-yl)-2,3-dihydrobenzofuran-7-yl)thieno[3,2-b]pyridin-2-yl)methyl)-2,2,2-trifluoroethan-1-amine

The title compound was synthesized using precursor III, general procedure G and D using N-((7-chlorothieno[3,2-b]pyridin-2-yl)methyl)-2,2,2-trifluoroethan-1-amine to give the desired product as the HCl salt. 1H NMR (400 MHz, CD3OD): δ ppm 8.97 (bs, 1H), 8.07, bs, 1H), 7.98 (bs, 1H), 7.74 (bs, 1H), 7.62 (bs, 1H), 6.28 (bs, 1H), 4.73 (bs, 2H), 4.17 (bs, 1H), 3.83 (bs, 2H), 3.74-3.30 (6H), 3.11 (bs, 2H). [M+H] 521.0.

### Example 324

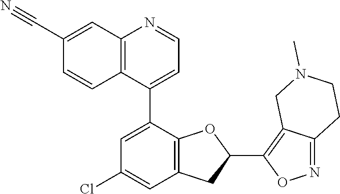 [SMILES: CN1CCc2noc([C@H]3Cc4cc(Cl)cc(-c5ccnc6cc(C#N)ccc56)c4O3)c2C1]

### (R)-4-(5-chloro-2-(5-methyl-4,5,6,7-tetrahydroisoxazolo[4,3-c]pyridin-3-yl)-2,3-dihydrobenzofuran-7-yl)quinoline-7-carbonitrile

The title compound was synthesized from reductive amination of the title compound from Example 300 using paraformaldehyde and sodium triacetoxyborohydride to give the desired product. 1H NMR (400 MHz, CD3OD) δ ppm 9.08 (d, J=4.4 Hz, 1H), 8.53 (d, J=1.3 Hz, 1H), 7.95 (d, J=8.7 Hz, 1H), 7.77 (d, J=8.4 Hz, 1H), 7.69 (d, J=4.4 Hz, 1H), 7.57-7.45 (m, 1H), 7.37 (d, J=2.2 Hz, 1H), 6.15-6.00 (m, 1H), 4.04 (s, 1H), 3.88 (dd, J=16.2, 10.1 Hz, 1H), 3.53-3.06 (m, 6H), 2.82 (s, 3H).

### Example 325

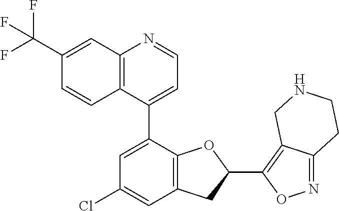 [SMILES: FC(F)(F)c1ccc2c(-c3cc(Cl)cc4c3O[C@@H](c3onc5c3CNCC5)C4)ccnc2c1]

### (R)-3-(5-chloro-7-(7-(trifluoromethyl)quinolin-4-yl)-2,3-dihydrobenzofuran-2-yl)-4,5,6,7-tetrahydroisoxazolo[4,3-c]pyridine

The title compound was synthesized using precursor III, general procedure C and D using 4-(4,4,5,5-tetramethyl-1,3,2-dioxaborolan-2-yl)-7-(trifluoromethyl)quinoline to give the desired product as the HCl salt. 1H NMR (400 MHz, CD3OD): δ ppm 9.41 (t, J=7.7 Hz, 1H), 8.62 (d, J=19.7 Hz, 1H), 8.32 (d, J=8.8 Hz, 1H), 8.20 (d, J=5.2 Hz, 1H), 8.09 (d, J=8.8 Hz, 1H), 7.63 (s, 1H), 7.50 (d, J=2.0 Hz, 1H), 6.23-6.08 (m, 1H), 4.06 (s, 1H), 3.92 (dd, J=16.6, 9.6 Hz, 1H), 3.45 (d, J=4.4 Hz, 2H), 3.30-3.01 (m, 4H).

### Example 326

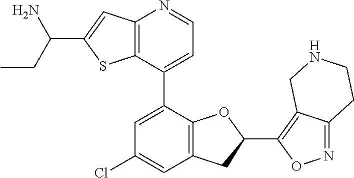 [SMILES: CCC(N)c1cc2nccc(-c3cc(Cl)cc4c3O[C@@H](c3onc5c3CNCC5)C4)c2s1]

### 1-(7-((R)-5-chloro-2-(4,5,6,7-tetrahydroisoxazolo[4,3-c]pyridin-3-yl)-2,3-dihydrobenzofuran-7-yl)thieno[3,2-b]pyridin-2-yl)propan-1-amine

The title compound was synthesized using precursor III, general procedure C and D using tert-butyl (1-(7-(4,4,5,5-tetramethyl-1,3,2-dioxaborolan-2-yl)thieno[3,2-b]pyridin-2-yl)propyl)carbamate to give the desired product as the HCl salt. 1H NMR (400 MHz, CD3OD): δ ppm 9.48 (bs, 1H), 8.10 (s, 1H), 8.02 (s, 1H), 7.74 (d, J=1.6 Hz, 1H), 7.63 (s, 1H), 6.29 (dd, J=9.6, 6.8 Hz, 1H), 4.20 (s, 2H), 3.94-3.42 (m, 5H), 3.12 (q, J=4.4 Hz, 2H), 2.21-2.11 (m, 2H), 1.05 (q, J=7.2 Hz, 3H). [M+H] 467.0.

### Example 327

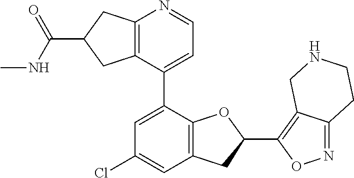 [SMILES: CNC(=O)C1Cc2nccc(-c3cc(Cl)cc4c3O[C@@H](c3onc5c3CNCC5)C4)c2C1]

### 4-((R)-5-chloro-2-(4,5,6,7-tetrahydroisoxazolo[4,3-c]pyridin-3-yl)-2,3-dihydrobenzofuran-7-yl)-N-methyl-6,7-dihydro-5H-cyclopenta[b]pyridine-6-carboxamide

The title compound was synthesized using precursor III, general procedure C using methyl 4-(4,4,5,5-tetramethyl-1,3,2-dioxaborolan-2-yl)-6,7-dihydro-5H-cyclopenta[b]pyridine-6-carboxylate. Ester hydrolysis with lithium hydroxide, amide coupling with methylamine and HATU, and general procedure E and F to give the desired product. 1H NMR (400 MHz, Methanol-d4) δ 8.56-8.43 (m, 1H), 7.73-7.64 (m, 1H), 7.55-7.44 (m, 1H), 7.40-7.34 (m, 1H), 6.30-6.15 (m, 1H), 4.26-3.96 (m, 2H), 3.84 (ddd, J=15.5, 10.0, 5.3 Hz, 1H), 3.63-3.04 (m, 10H), 2.79-2.69 (m, 3H). [M+H] 451.0.

### Example 328

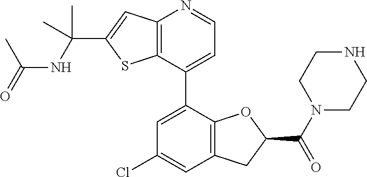 [SMILES: CC(=O)NC(C)(C)c1cc2nccc(-c3cc(Cl)cc4c3O[C@@H](C(=O)N3CCNCC3)C4)c2s1]

### (R)-N-(2-(7-(5-chloro-2-(piperazine-1-carbonyl)-2,3-dihydrobenzofuran-7-yl)thieno[3,2-b]pyridin-2-yl)propan-2-yl)acetamide

The title compound was synthesized using precursor II, general procedure B, E and F using N-(2-(7-chlorothieno[3,2-b]pyridin-2-yl)propan-2-yl)acetamide to give the desired product. 1H NMR (400 MHz, Methanol-d4) δ 8.66 (d, J=5.2 Hz, 1H), 7.56 (d, J=5.2 Hz, 1H), 7.49-7.44 (m, 2H), 7.40 (s, 1H), 5.76 (dd, J=10.0, 7.1 Hz, 1H), 4.12-3.96 (m, 1H), 3.87 (dd, J=16.4, 7.0 Hz, 2H), 3.76-3.43 (m, 3H), 3.33-2.96 (m, 3H), 2.80-2.59 (m, 1H), 1.97 (d, J=6.7 Hz, 3H), 1.84-1.65 (m, 6H). [M+H] 499.0.

### Example 329

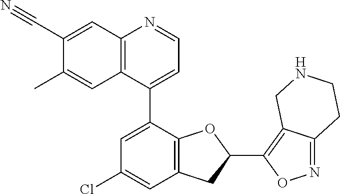 [SMILES: Cc1cc2c(-c3cc(Cl)cc4c3O[C@@H](c3onc5c3CNCC5)C4)ccnc2cc1C#N]

### (R)-4-(5-chloro-2-(4,5,6,7-tetrahydroisoxazolo[4,3-c]pyridin-3-yl)-2,3-dihydrobenzofuran-7-yl)-6-methylquinoline-7-carbonitrile

The title compound was synthesized using precursor III, general procedure G and E using 4-chloro-6-methylquinoline-7-carbonitrile to give the desired product as the TFA salt. 1H NMR (400 MHz, CD3OD): δ ppm 8.98 (d, J=4.5 Hz, 1H), 8.45 (s, 1H), 7.71 (s, 1H), 7.62 (d, J=4.4 Hz, 1H), 7.57-7.45 (m, 1H), 7.33 (d, J=2.2 Hz, 1H), 6.08 (s, 1H), 4.04 (s, 1H), 3.96-3.78 (m, 1H), 3.58 (dd, J=16.2, 6.4 Hz, 2H), 3.45 (t, J=6.2 Hz, 2H), 3.08 (t, J=6.1 Hz, 2H), 2.60 (s, 3H). [M+H] 443.0.

### Example 330

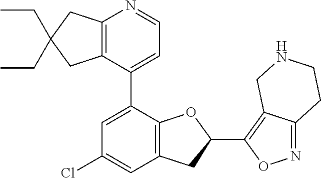 [SMILES: CCC1(CC)Cc2nccc(-c3cc(Cl)cc4c3O[C@@H](c3onc5c3CNCC5)C4)c2C1]

### (R)-3-(5-chloro-7-(6,6-diethyl-6,7-dihydro-5H-cyclopenta[b]pyridin-4-yl)-2,3-dihydrobenzofuran-2-yl)-4,5,6,7-tetrahydroisoxazolo[4,3-c]pyridine

The title compound was synthesized using precursor III, general procedure C and D using 6,6-diethyl-4-(4,4,5,5-tetramethyl-1,3,2-dioxaborolan-2-yl)-6,7-dihydro-5H-cyclopenta[b]pyridine to give the desired product as the HCl salt. 1H NMR (400 MHz, Methanol-d4) δ 8.39 (s, 1H), 7.46 (s, 2H), 7.31 (s, 1H), 6.18 (s, 1H), 4.16 (s, 2H), 3.91-2.60 (m, 10H), 1.51 (dd, J=23.1, 7.4 Hz, 4H), 0.83 (dt, J=22.9, 7.4 Hz, 6H). [M+H] 450.1.

### Example 331

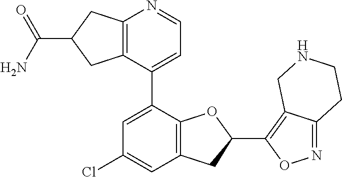 [SMILES: NC(=O)C1Cc2nccc(-c3cc(Cl)cc4c3O[C@@H](c3onc5c3CNCC5)C4)c2C1]

### 4-((R)-5-chloro-2-(4,5,6,7-tetrahydroisoxazolo[4,3-c]pyridin-3-yl)-2,3-dihydrobenzofuran-7-yl)-6,7-dihydro-5H-cyclopenta[b]pyridine-6-carboxamide

The intermediate from Example 327 was treated with ammonia and HATU, and general procedure E and F to give the desired product. 1H NMR (400 MHz, Methanol-d4) δ 8.48 (s, 1H), 7.68-7.54 (m, 1H), 7.53-7.42 (m, 1H), 7.42-7.21 (m, 1H), 6.21 (dd, J=9.9, 5.2 Hz, 1H), 4.24-3.99 (m, 2H), 3.84 (dd, J=16.4, 9.9 Hz, 1H), 3.69-3.01 (m, 10H). [M+H] 437.0.

### Example 332

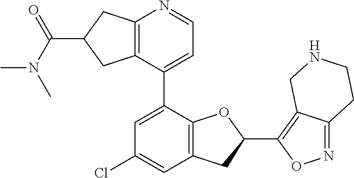 [SMILES: CN(C)C(=O)C1Cc2nccc(-c3cc(Cl)cc4c3O[C@@H](c3onc5c3CNCC5)C4)c2C1]

### 4-((R)-5-chloro-2-(4,5,6,7-tetrahydroisoxazolo[4,3-c]pyridin-3-yl)-2,3-dihydrobenzofuran-7-yl)-N,N-dimethyl-6,7-dihydro-5H-cyclopenta[b]pyridine-6-carboxamide

The intermediate from Example 327 was treated with dimethylamine and HATU, and general procedure E and F to give the desired product. 1H NMR (400 MHz, Methanol-d4) δ 8.54-8.45 (m, 1H), 7.70-7.60 (m, 1H), 7.54-7.46 (m, 1H), 7.42-7.34 (m, 1H), 6.28-6.14 (m, 1H), 4.25-3.97 (m, 2H), 3.95-3.78 (m, 1H), 3.66-3.20 (m, 8H), 3.19-3.12 (m, 3H), 3.12-3.02 (m, 2H), 3.01-2.87 (m, 3H). [M+H] 465.0.

### Example 333

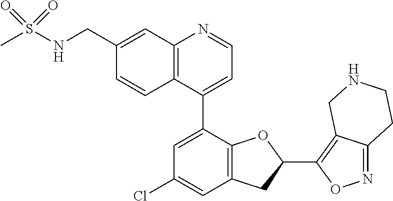 [SMILES: CS(=O)(=O)NCc1ccc2c(-c3cc(Cl)cc4c3O[C@@H](c3onc5c3CNCC5)C4)ccnc2c1]

### (R)-N-((4-(5-chloro-2-(4,5,6,7-tetrahydroisoxazolo[4,3-c]pyridin-3-yl)-2,3-dihydrobenzofuran-7-yl)quinolin-7-yl)methyl)methanesulfonamide

The title compound was synthesized using precursor III, general procedure C and D using 2-((4-(4,4,5,5-tetramethyl-1,3,2-dioxazolidin-2-yl)quinolin-7-yl)methyl)isoindoline-1,3-dione. The phthalimide protecting group was removed with hydrazine, and treatment with methanesulfonylchloride followed by procedure D to give the desired product as the HCl salt. 1H NMR (400 MHz, CD3OD): δ ppm 9.24 (s, 1H), 8.31 (s, 1H), 8.10-8.03 (m, 2H), 7.92 (s, 1H), 7.63 (s, 1H), 7.49 (s, 1H), 6.15 (s, 1H), 4.61 (s, 2H), 4.05 (s, 2H), 3.89 (s, 1H), 3.48 (s, 2H), 3.11 (s, 2H), 3.06 (s, 3H).

### Example 334

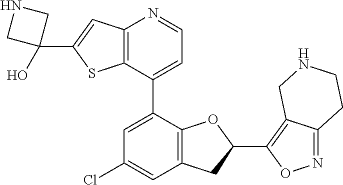 [SMILES: OC1(c2cc3nccc(-c4cc(Cl)cc5c4O[C@@H](c4onc6c4CNCC6)C5)c3s2)CNC1]

### (R)-3-(7-(5-chloro-2-(4,5,6,7-tetrahydroisoxazolo[4,3-c]pyridin-3-yl)-2,3-dihydrobenzofuran-7-yl)thieno[3,2-b]pyridin-2-yl)azetidin-3-ol

The title compound was synthesized using precursor III, general procedure C and D using tert-butyl 3-hydroxy-3-(7-(4,4,5,5-tetramethyl-1,3,2-dioxaborolan-2-yl)thieno[3,2-b]pyri din-2-yl)azetidine-1-carboxylate to give the desired product as the HCl salt. 1H NMR (400 MHz, CD3OD): δ ppm 8.68 (d, J=4.8 Hz, 1H), 7.69 (s, 1H), 7.50 (s, 1H), 7.42 (s, 1H), 7.41 (s, 1H), 6.09 (dd, J=9.2, 5.6 Hz, 1H), 4.33 (t, J=8.8 Hz, 2H), 4.15 (d, J=11.2 Hz, 2H), 3.84-3.52 (m, 4H), 2.96-2.93 (m, 2H), 2.74 (t, J=6.0 Hz, 2H). [M+H] 481.0.

### Example 335

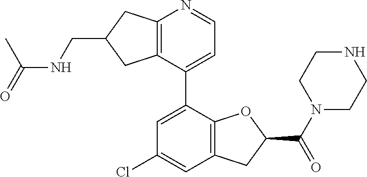 [SMILES: CC(=O)NCC1Cc2nccc(-c3cc(Cl)cc4c3O[C@@H](C(=O)N3CCNCC3)C4)c2C1]

### N-((4-((R)-5-chloro-2-(piperazine-1-carbonyl)-2,3-dihydrobenzofuran-7-yl)-6,7-dihydro-5H-cyclopenta[b]pyridin-6-yl)methyl)acetamide

The title compound was synthesized using precursor II, general procedure B using 2-((4-chloro-6,7-dihydro-5H-cyclopenta[b]pyridin-6-yl)methyl)isoindoline-1,3-dione. The phthalimide protecting group was removed with hydrazine, and treatment with acetic anhydride followed by procedure D to give the desired product as the HCl salt. 1H NMR (400 MHz, Methanol-d4) δ 8.53-8.43 (m, 1H), 7.85-7.72 (m, 1H), 7.47-7.39 (m, 1H), 7.37-7.30 (m, 1H), 5.89-5.74 (m, 1H), 3.86 (s, 4H), 3.72-3.49 (m, 2H), 3.45-3.16 (m, 8H), 3.16-2.99 (m, 2H), 2.97-2.72 (m, 1H), 1.99-1.83 (m, 3H). [M+H] 455.1.

### Example 336

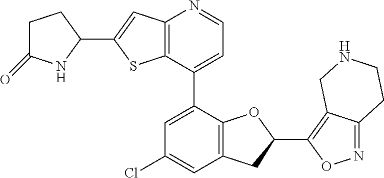 [SMILES: O=C1CCC(c2cc3nccc(-c4cc(Cl)cc5c4O[C@@H](c4onc6c4CNCC6)C5)c3s2)N1]

### 5-(7-((R)-5-chloro-2-(4,5,6,7-tetrahydroisoxazolo[4,3-c]pyridin-3-yl)-2,3-dihydrobenzofuran-7-yl)thieno[3,2-b]pyridin-2-yl)pyrrolidin-2-one

The title compound was synthesized using precursor III, general procedure C and D using 5-(7-(4,4,5,5-tetramethyl-1,3,2-dioxaborolan-2-yl)thieno[3,2-b]pyridin-2-yl)pyrrolidin-2-one to give the desired product as the HCl salt. 1H NMR (400 MHz, CD3OD): δ ppm 8.95 (bs, 1H), 8.02 (s, 1H), 7.90 (s, 1H), 7.70 (s, 1H), 7.69 (s, 1H), 7.63 (s, 1H), 6.24 (dd, J=10.0, 6.4 Hz, 1H), 5.34 (q, J=8.0 Hz, 1H), 4.24-4.4.10 (m, 2H), 3.92-3.52 (m, 5H), 2.84-2.76 (m, 1H), 2.58-2.44 (m, 2H), 2.22-2.15 (m, 1H). [M+H] 493.0.

### Example 337

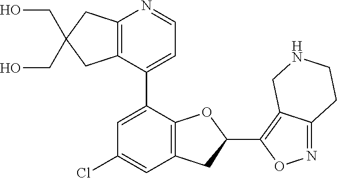 [SMILES: OCC1(CO)Cc2nccc(-c3cc(Cl)cc4c3O[C@@H](c3onc5c3CNCC5)C4)c2C1]

### (R)-(4-(5-chloro-2-(4,5,6,7-tetrahydroisoxazolo[4,3-c]pyridin-3-yl)-2,3-dihydrobenzofuran-7-yl)-6,7-dihydro-5H-cyclopenta[b]pyridine-6,6-diyl)dimethanol

The title compound was synthesized using precursor III, general procedure C using (4-(4,4,5,5-tetramethyl-1,3,2-dioxaborolan-2-yl)-6,7-dihydro-5H-cyclopenta[b]pyridine-6,6-diyl)bis(methylene) diacetate. The esters were hydrolyzed using lithium hydroxide and general procedure D to give the desired product as the HCl salt. 1H NMR (400 MHz, Methanol-d4) δ 8.54-8.45 (m, 1H), 7.75-7.68 (m, 1H), 7.50 (s, 1H), 7.41 (s, 1H), 6.27-6.13 (m, 1H), 4.16 (s, 2H), 3.92-3.41 (m, 8H), 3.26-2.79 (m, 6H). [M+H] 454.0.

### Example 338

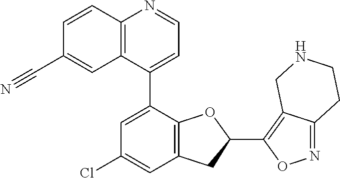 [SMILES: N#Cc1ccc2nccc(-c3cc(Cl)cc4c3O[C@@H](c3onc5c3CNCC5)C4)c2c1]

### (R)-4-(5-chloro-2-(4,5,6,7-tetrahydroisoxazolo[4,3-c]pyridin-3-yl)-2,3-dihydrobenzofuran-7-yl)quinoline-6-carbonitrile

The title compound was synthesized using precursor III, general procedure C and D using 4-(4,4,5,5-tetramethyl-1,3,2-dioxaborolan-2-yl)quinoline-6-carbonitrile to give the desired product as the HCl salt. 1H NMR (400 MHz, CD3OD): δ ppm 9.35 (d, J=5.4 Hz, 1H), 8.39 (d, J=8.8 Hz, 2H), 8.28 (d, J=8.7 Hz, 1H), 8.10 (d, J=5.5 Hz, 1H), 7.66 (d, J=9.0 Hz, 1H), 7.52 (t, J=8.9 Hz, 1H), 6.32-6.05 (m, 1H), 4.10 (s, 1H), 3.99-3.79 (m, 1H), 3.82-3.19 (m, 4H), 3.11 (d, J=6.2 Hz, 1H). [M+H] 429.1.

### Example 339

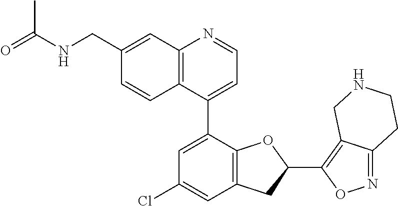 [SMILES: CC(=O)NCc1ccc2c(-c3cc(Cl)cc4c3O[C@@H](c3onc5c3CNCC5)C4)ccnc2c1]

### (R)-N-((4-(5-chloro-2-(4,5,6,7-tetrahydroisoxazolo[4,3-c]pyridin-3-yl)-2,3-dihydrobenzofuran-7-yl)quinolin-7-yl)methyl)acetamide

The intermediate from Example 333 was treated with acetic anhydride and general procedure D to give the desired product as the HCl salt. 1H NMR (400 MHz, CD3OD): δ ppm 9.23 (d, J=5.1 Hz, 1H), 8.15 (s, 1H), 8.12-7.98 (m, 2H), 7.83 (d, J=8.5 Hz, 1H), 7.62 (s, 1H), 7.49 (s, 1H), 6.14 (s, 1H), 4.67 (s, 2H), 4.03 (s, 2H), 3.88 (d, J=9.4 Hz, 1H), 3.70-3.61 (m, 1H), 3.47 (s, 2H), 3.11 (s, 2H), 2.09 (s, 3H).

### Example 340

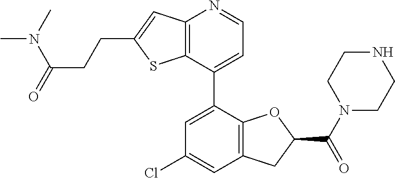 [SMILES: CN(C)C(=O)CCc1cc2nccc(-c3cc(Cl)cc4c3O[C@@H](C(=O)N3CCNCC3)C4)c2s1]

### (R)-3-(7-(5-chloro-2-(piperazine-1-carbonyl)-2,3-dihydrobenzofuran-7-yl)thieno[3,2-b]pyridin-2-yl)-N,N-dimethylpropanamide

The intermediate from Example 313 was coupled to dimethylamine using HATU, and general procedure D to give the desired compound as the HCl salt. [M+H] 499.0

### Example 341

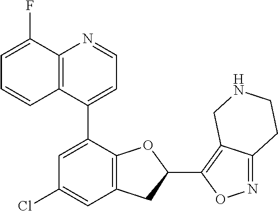 [SMILES: Fc1cccc2c(-c3cc(Cl)cc4c3O[C@@H](c3onc5c3CNCC5)C4)ccnc12]

### (R)-3-(5-chloro-7-(8-fluoroquinolin-4-yl)-2,3-dihydrobenzofuran-2-yl)-4,5,6,7-tetrahydroisoxazolo[4,3-c]pyridine

The title compound was synthesized using precursor III, general procedure C and D using 8-fluoro-4-(4,4,5,5-tetramethyl-1,3,2-dioxaborolan-2-yl)quinoline to give the desired product as the HCl salt. 1H NMR (400 MHz, CD3OD): δ ppm 9.25 (d, J=4.6 Hz, 1H), 8.14 (d, J=4.9 Hz, 1H), 7.92 (dt, J=18.9, 9.5 Hz, 2H), 7.61 (s, 1H), 7.47 (s, 1H), 6.16 (s, 1H), 4.06 (s, 1H), 3.91 (dd, J=16.2, 8.9 Hz, 1H), 3.59-3.36 (m, 4H), 3.14 (d, J=42.4 Hz, 2H).

### Example 342

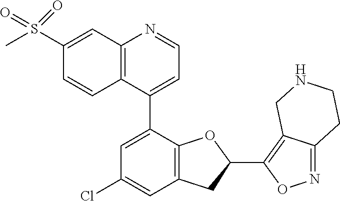 [SMILES: CS(=O)(=O)c1ccc2c(-c3cc(Cl)cc4c3O[C@@H](c3onc5c3CNCC5)C4)ccnc2c1]

### (R)-3-(5-chloro-7-(7-(methylsulfonyl)quinolin-4-yl)-2,3-dihydrobenzofuran-2-yl)-4,5,6,7-tetrahydroisoxazolo[4,3-c]pyridine

The title compound was synthesized using precursor III, general procedure G and D using 4-chloro-7-(methylsulfonyl)quinoline to give the desired product as the HCl salt. 1H NMR (400 MHz, CD3OD): δ ppm 9.44 (d, J=5.4 Hz, 1H), 8.84 (s, 1H), 8.30 (q, J=9.1 Hz, 2H), 8.24 (d, J=5.3 Hz, 1H), 7.63 (s, 1H), 7.51 (d, J=2.0 Hz, 1H), 6.19 (dd, J=9.8, 6.4 Hz, 1H), 4.11-4.07 (m, 2H), 3.97-3.82 (m, 1H), 3.76-3.52 (m, 5H), 3.52-3.39 (m, 2H), 3.18-3.01 (m, 2H).

### Example 343

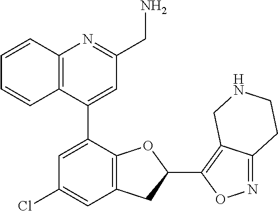 [SMILES: NCc1cc(-c2cc(Cl)cc3c2O[C@@H](c2onc4c2CNCC4)C3)c2ccccc2n1]

### (R)-(4-(5-chloro-2-(4,5,6,7-tetrahydroisoxazolo[4,3-c]pyridin-3-yl)-2,3-dihydrobenzofuran-7-yl)quinolin-2-yl)methanamine

The title compound was synthesized using precursor III, general procedure C and D using 2-((4-(4,4,5,5-tetramethyl-1,3,2-dioxazolidin-2-yl)quinolin-7-yl)methyl)isoindoline-1,3-dione. The phthalimide protecting group was removed with hydrazine followed by procedure D to give the desired product as the HCl salt. 1H NMR (400 MHz, CD3OD): δ ppm 9.26 (d, J=5.5 Hz, 1H), 8.37 (s, 1H), 8.15 (d, J=8.9 Hz, 1H), 8.07 (d, J=5.5 Hz, 1H), 7.95 (d, J=9.3 Hz, 1H), 7.62 (s, 1H), 7.47 (d, J=2.1 Hz, 1H), 6.21-6.08 (m, 1H), 4.50 (s, 2H), 4.08 (s, 2H), 3.91 (dd, J=16.5, 10.3 Hz, 1H), 3.68-3.54 (m, 1H), 3.47 (t, J=6.4 Hz, 2H), 3.11 (s, 2H). [M+H] 433.0.

### Example 344

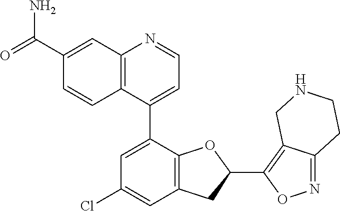 [SMILES: NC(=O)c1ccc2c(-c3cc(Cl)cc4c3O[C@@H](c3onc5c3CNCC5)C4)ccnc2c1]

### (R)-4-(5-chloro-2-(4,5,6,7-tetrahydroisoxazolo[4,3-c]pyridin-3-yl)-2,3-dihydrobenzofuran-7-yl)quinoline-7-carboxamide

The title compound was synthesized using precursor III, general procedure C and D using 4-(4,4,5,5-tetramethyl-1,3l2-oxaborolan-2-yl)quinoline-7-carboxamide to give the desired product as the HCl salt. 1H NMR (400 MHz, CD3OD): δ ppm 9.34 (s, 1H), 8.73 (s, 1H), 8.25 (s, 1H), 8.19 (m, 2H), 7.64 (s, 1H), 7.51 (s, 1H), 6.17 (s, 1H), 4.06 (d, J=7.7 Hz, 2H), 3.91 (m, 1H), 3.69-3.17 (m, 5H).

### Example 345

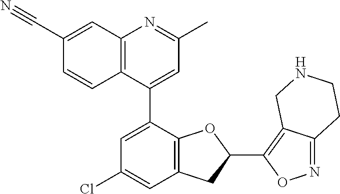 [SMILES: Cc1cc(-c2cc(Cl)cc3c2O[C@@H](c2onc4c2CNCC4)C3)c2ccc(C#N)cc2n1]

### (R)-4-(5-chloro-2-(4,5,6,7-tetrahydroisoxazolo[4,3-c]pyridin-3-yl)-2,3-dihydrobenzofuran-7-yl)-2-methylquinoline-7-carbonitrile

The title compound was synthesized using precursor III, general procedure G and E using 4-chloro-2-methylquinoline-7-carbonitrile to give the desired product as the TFA salt. 1H NMR (400 MHz, CD3OD): δ ppm 8.39 (d, J=1.2 Hz, 1H), 7.87 (d, J=8.8 Hz, 1H), 7.70 (s, 1H), 7.60 (s, 1H), 7.52 (d, J=2.1 Hz, 1H), 7.34 (d, J=2.2 Hz, 1H), 6.13-5.98 (m, 1H), 4.00 (s, 1H), 3.87 (dd, J=16.1, 9.9 Hz, 1H), 3.57 (dd, J=16.5, 6.6 Hz, 2H), 3.44 (t, J=6.3 Hz, 2H), 3.07 (t, J=6.3 Hz, 2H), 2.80 (s, 3H).

### Example 346

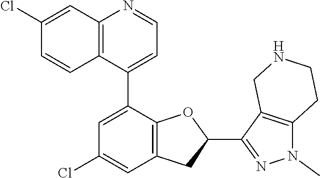 [SMILES: Cn1nc([C@H]2Cc3cc(Cl)cc(-c4ccnc5cc(Cl)ccc45)c3O2)c2c1CCNC2]

### (R)-7-chloro-4-(5-chloro-2-(1-methyl-4,5,6,7-tetrahydro-1H-pyrazolo[4,3-c]pyridin-3-yl)-2,3-dihydrobenzofuran-7-yl)quinoline

The title compound was synthesized using precursor IV, general procedure C and D using 7-chloro-4-(4,4,5,5-tetramethyl-1,3,2-dioxaborolan-2-yl)quinoline to give the desired product as the HCl salt. 1H NMR (400 MHz, CD3OD): δ ppm 9.31 (d, J=5.7 Hz, 1H), 8.34 (d, J=1.9 Hz, 1H), 8.15 (d, J=5.7 Hz, 1H), 8.11 (d, J=9.2 Hz, 1H), 7.94 (d, J=9.3 Hz, 1H), 7.64 (d, J=2.0 Hz, 1H), 7.51 (d, J=2.1 Hz, 1H), 6.26-6.14 (m, 1H), 4.13 (d, J=14.2 Hz, 1H), 3.92 (dd, J=15.7, 10.3 Hz, 1H), 3.76 (s, 3H), 3.75-3.70 (m, 2H), 3.69-3.62 (m, 2H), 2.93 (t, J=6.2 Hz, 2H). [M+H] 451.0.

### Example 347

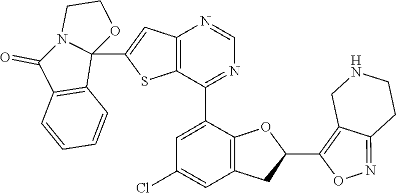 [SMILES: O=C1c2ccccc2C2(c3cc4ncnc(-c5cc(Cl)cc6c5O[C@@H](c5onc7c5CNCC7)C6)c4s3)OCCN12]

### 9b-(4-((R)-5-chloro-2-(4,5,6,7-tetrahydroisoxazolo[4,3-c]pyridin-3-yl)-2,3-dihydrobenzofuran-7-yl)thieno[3,2-d]pyrimidin-6-yl)-2,3-dihydrooxazolo[2,3-a]isoindol-5(9bH)-one

The title compound was synthesized using precursor III, general procedure G and D using 9b-(4-chlorothieno[3,2-d]pyrimidin-6-yl)-2,3-dihydrooxazolo[2,3-a]isoindol-5(9bH)-one to give the desired product as the HCl salt. 1H NMR (400 MHz, CD3OD): δ ppm 9.27 (s, 1H), 7.85 (d, J=6.2 Hz, 1H), 7.79-7.38 (m, 5H), 7.31 (s, 1H), 6.25 (dd, J=10.0, 5.4 Hz, 1H), 4.23 (s, 1H), 3.96-3.71 (m, 3H), 3.71-3.40 (m, 6H), 3.27-3.02 (m, 2H).

### Example 348

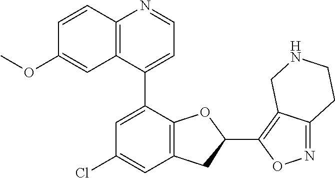 [SMILES: COc1ccc2nccc(-c3cc(Cl)cc4c3O[C@@H](c3onc5c3CNCC5)C4)c2c1]

### (R)-3-(5-chloro-7-(6-methoxyquinolin-4-yl)-2,3-dihydrobenzofuran-2-yl)-4,5,6,7-tetrahydroisoxazolo[4,3-c]pyridine

The title compound was synthesized using precursor III, general procedure C and D using 6-methoxy-4-(4,4,5,5-tetramethyl-1,3,2-dioxaborolan-2-yl)quinoline to give the desired product as the HCl salt. 1H NMR (400 MHz, CD3OD): δ ppm 8.79 (d, J=4.6 Hz, 1H), 7.60 (d, J=9.0 Hz, 1H), 7.48-7.31 (m, 3H), 7.25 (s, 1H), 7.14 (s, 1H), 6.00 (s, 1H), 4.13 (dt, J=14.4, 11.6 Hz, 2H), 3.95 (s, 3H), 3.85-3.36 (m, 4H), 2.73 (d, J=5.4 Hz, 2H). [M+H] 434.0.

### Example 349

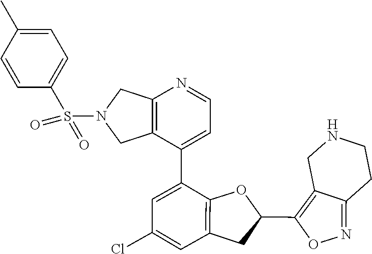 [SMILES: Cc1ccc(S(=O)(=O)N2Cc3nccc(-c4cc(Cl)cc5c4O[C@@H](c4onc6c4CNCC6)C5)c3C2)cc1]

### (R)-3-(5-chloro-7-(6-tosyl-6,7-dihydro-5H-pyrrolo[3,4-b]pyridin-4-yl)-2,3-dihydrobenzofuran-2-yl)-4,5,6,7-tetrahydroisoxazolo[4,3-c]pyridine

The title compound was synthesized using precursor III, general procedure G, E and F using 4-chloro-6-tosyl-6,7-dihydro-5H-pyrrolo[3,4-b]pyridine to give the desired product. 1H NMR (400 MHz, Methanol-d) δ 8.42 (d, J=5.4 Hz, 1H), 7.65 (d, J=8.3 Hz, 2H), 7.44 (s, 1H), 7.35 (d, J=8.1 Hz, 3H), 7.28 (d, J=5.2 Hz, 1H), 7.21 (s, 1H), 6.13 (dd, J=8, 4 Hz, 1H), 4.56 (d, J=10.4 Hz, 5H), 3.81-3.76 (m, 1H), 3.63 (d, J=18.7 Hz, 2H), 3.02 (t, J=5.9 Hz, 2H), 2.83-2.74 (m, 2H), 2.38 (s, 3H). [M+H] 549.0.

### Example 350

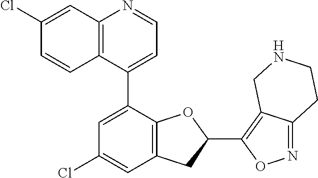 [SMILES: Clc1cc2c(c(-c3ccnc4cc(Cl)ccc34)c1)O[C@@H](c1onc3c1CNCC3)C2]

### (R)-3-(5-chloro-7-(7-chloroquinolin-4-yl)-2,3-dihydrobenzofuran-2-yl)-4,5,6,7-tetrahydroisoxazolo[4,3-c]pyridine

The title compound was synthesized using precursor III, general procedure C and D using 7-chloro-4-(4,4,5,5-tetramethyl-1,3,2-dioxaborolan-2-yl)quinoline to give the desired product as the HCl salt. 1H NMR (400 MHz, CD3OD): δ ppm 9.29 (d, J=5.6 Hz, 1H), 8.33 (d, J=1.9 Hz, 1H), 8.12 (dd, J=11.0, 7.4 Hz, 2H), 7.90 (d, J=9.0 Hz, 1H), 7.69-7.56 (m, 1H), 7.49 (d, J=2.2 Hz, 1H), 6.18 (dd, J=9.9, 6.4 Hz, 1H), 4.19-4.01 (m, 2H), 4.01-3.81 (m, 1H), 3.81-3.10 (m, 5H).

### Example 351

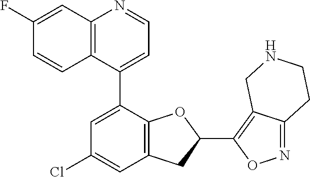 [SMILES: Fc1ccc2c(-c3cc(Cl)cc4c3O[C@@H](c3onc5c3CNCC5)C4)ccnc2c1]

### (R)-3-(5-chloro-7-(7-fluoroquinolin-4-yl)-2,3-dihydrobenzofuran-2-yl)-4,5,6,7-tetrahydroisoxazolo[4,3-c]pyridine

The title compound was synthesized using precursor III, general procedure C and D using 7-fluoro-4-(4,4,5,5-tetramethyl-1,3,2-dioxaborolan-2-yl)quinoline to give the desired product as the HCl salt. 1H NMR (400 MHz, CD3OD): δ ppm 9.30 (s, 1H), 8.24 (s, 1H), 8.12 (s, 1H), 8.04 (d, J=8.4 Hz, 1H), 7.79 (s, 1H), 7.63 (s, 1H), 7.52 (d, J=20.1 Hz, 1H), 6.19 (s, 1H), 4.09 (s, 1H), 4.01-3.84 (m, 1H), 3.84-3.54 (m, 2H), 3.47 (s, 2H), 3.10 (s, 2H). [M+H] 422.0.

### Example 352

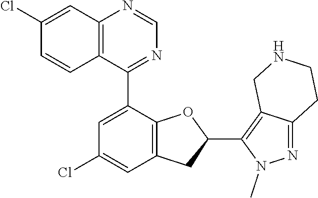 [SMILES: Cn1nc2c(c1[C@H]1Cc3cc(Cl)cc(-c4ncnc5cc(Cl)ccc45)c3O1)CNCC2]

### (R)-7-chloro-4-(5-chloro-2-(2-methyl-4,5,6,7-tetrahydro-2H-pyrazolo[4,3-c]pyridin-3-yl)-2,3-dihydrobenzofuran-7-yl)quinazoline

The title compound was synthesized using precursor IV, general procedure C and D using 7-chloro-4-(4,4,5,5-tetramethyl-1,3,2-dioxaborolan-2-yl)quinoline to give the desired product as the HCl salt. 1H NMR (400 MHz, CD3OD): δ ppm 9.29 (d, J=5.4 Hz, 1H), 8.35 (d, J=1.6 Hz, 1H), 8.18 (d, J=9.1 Hz, 1H), 8.13 (d, J=5.1 Hz, 1H), 7.94 (d, J=8.1 Hz, 1H), 7.58 (s, 1H), 7.45 (d, J=1.8 Hz, 1H), 5.97 (t, J=8.5 Hz, 1H), 3.98 (d, J=14.4 Hz, 1H), 3.88 (d, J=14.3 Hz, 1H), 3.78-3.67 (m, 5H), 3.62-3.54 (m, 2H), 2.99 (s, 2H). [M+H] 450.9.

### Example 353

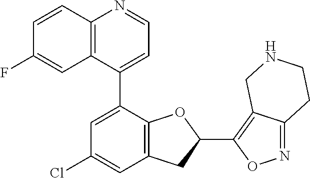 [SMILES: Fc1ccc2nccc(-c3cc(Cl)cc4c3O[C@@H](c3onc5c3CNCC5)C4)c2c1]

### (R)-3-(5-chloro-7-(6-fluoroquinolin-4-yl)-2,3-dihydrobenzofuran-2-yl)-4,5,6,7-tetrahydroisoxazolo[4,3-c]pyridine

The title compound was synthesized using precursor III, general procedure C and D using 6-fluoro-4-(4,4,5,5-tetramethyl-1,3,2-dioxaborolan-2-yl)quinoline to give the desired product as the HCl salt. [M+H] 422.1.

### Example 354

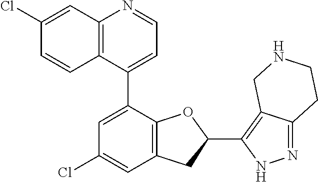 [SMILES: Clc1cc2c(c(-c3ccnc4cc(Cl)ccc34)c1)O[C@@H](c1[nH]nc3c1CNCC3)C2]

### (R)-7-chloro-4-(5-chloro-2-(4,5,6,7-tetrahydro-2H-pyrazolo[4,3-c]pyridin-3-yl)-2,3-dihydrobenzofuran-7-yl)quinoline

The title compound was synthesized using precursor IV, general procedure C and D using 7-chloro-4-(4,4,5,5-tetramethyl-1,3,2-dioxaborolan-2-yl)quinoline to give the desired product as the HCl salt. 1H NMR (400 MHz, CD3OD): δ ppm 9.28 (d, J=5.7 Hz, 1H), 8.32 (d, J=1.9 Hz, 1H), 8.15 (dd, J=19.5, 7.4 Hz, 2H), 7.92 (d, J=9.0 Hz, 1H), 7.70-7.52 (m, 2H), 7.46 (d, J=2.0 Hz, 1H), 6.01 (t, J=8.3 Hz, 1H), 3.88-3.70 (m, 2H), 3.62-3.36 (m, 4H), 3.01 (t, J=5.9 Hz, 2H). [M+H] 437.0.

### Example 355

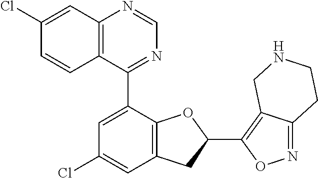 [SMILES: Clc1cc2c(c(-c3ncnc4cc(Cl)ccc34)c1)O[C@@H](c1onc3c1CNCC3)C2]

### (R)-3-(5-chloro-7-(7-chloroquinazolin-4-yl)-2,3-dihydrobenzofuran-2-yl)-4,5,6,7-tetrahydroisoxazolo[4,3-c]pyridine

The title compound was synthesized using precursor III, general procedure G and D using 4,7-dichloroquinazoline to give the desired product as the HCl salt. [M+H] 430.0.

### Example 356

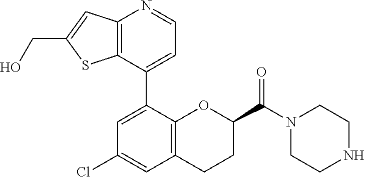 [SMILES: O=C([C@H]1CCc2cc(Cl)cc(-c3ccnc4cc(CO)sc34)c2O1)N1CCNCC1]

### (R)-(6-chloro-8-(2-(hydroxymethyl)thieno[3,2-b]pyridin-7-yl)chroman-2-yl)(piperazin-1-yl)methanone

The title compound was synthesized using precursor IX, general procedure C and D using (7-(4,4,5,5-tetramethyl-1,3,2-dioxaborolan-2-yl)thieno[3,2-b]pyridin-2-yl)methanol to give the desired product as the HCl salt. 1H NMR (400 MHz, CD3OD): δ ppm 8.85 (d, J=6.0 Hz, 1H), 7.95 (d, J=6.0 Hz, 1H), 7.67-7.66 (m, 1H), 7.48 (d, J=2.5 Hz, 1H), 7.41 (d, J=2.6 Hz, 1H), 5.28 (dd, J=7.2, 3.7 Hz, 1H), 5.04 (d, J=1.2 Hz, 2H), 3.81-3.70 (m, 4H), 3.22-3.10 (m, 4H), 3.09-2.86 (m, 2H), 2.33-2.10 (m, 2H). [M+H] 444.0.

### Example 357

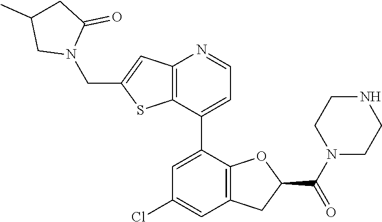 [SMILES: CC1CC(=O)N(Cc2cc3nccc(-c4cc(Cl)cc5c4O[C@@H](C(=O)N4CCNCC4)C5)c3s2)C1]

### 1-((7-((R)-5-chloro-2-(piperazine-1-carbonyl)-2,3-dihydrobenzofuran-7-yl)thieno[3,2-b]pyridin-2-yl)methyl)-4-methylpyrrolidin-2-one

The title compound was synthesized using precursor II, general procedure B and D using 1-((7-chlorothieno[3,2-b]pyridin-2-yl)methyl)-4-methylpyrrolidin-2-one to give the desired product as the HCl salt. 1H NMR (400 MHz, Methanol-d4) δ 8.90 (d, J=5.8 Hz, 1H), 8.09 (d, J=6.0 Hz, 1H), 7.72 (s, 1H), 7.64 (d, J=2.1 Hz, 1H), 7.54 (d, J=1.5 Hz, 1H), 5.88 (t, J=8.5 Hz, 1H), 4.92 (s, 2H), 3.88-3.38 (m, 12H), 2.14-2.07 (m, 1H), 1.69-1.62 (m, 1H), 1.42-1.34 (m, 1H), 1.13 (d, J=6.7 Hz, 3H). [M+H] 511.0.

### Example 358

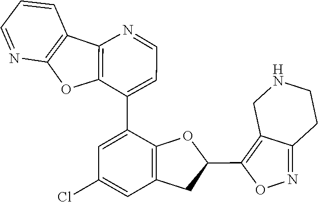 [SMILES: Clc1cc2c(c(-c3ccnc4c3oc3ncccc34)c1)O[C@@H](c1onc3c1CNCC3)C2]

### (R)-4-(5-chloro-2-(4,5,6,7-tetrahydroisoxazolo[4,3-c]pyridin-3-yl)-2,3-dihydrobenzofuran-7-yl)furo[2,3-b:4,5-b′]dipyridine

The title compound was synthesized using precursor III, general procedure G and D using 4-chlorofuro[2,3-b:4,5-b′]dipyridine to give the desired product as the HCl salt. 1H NMR (400 MHz, CD3OD) δ 9.32 (s, 1H), 8.88-8.72 (m, 2H), 7.92 (d, J=2.2 Hz, 1H), 7.71 (dd, J=7.7, 4.9 Hz, 1H), 7.61-7.52 (m, 1H), 6.24 (dd, J=9.9, 7.0 Hz, 1H), 4.67 (d, J=15.4 Hz, 1H), 4.41 (d, J=15.5 Hz, 1H), 3.97-3.85 (m, 1H), 3.74 (ddd, J=6.1, 5.2, 0.9 Hz, 1H), 3.68 (d, J=0.7 Hz, 1H), 3.60-3.52 (m, 1H), 3.15 (t, J=6.4 Hz, 2H). [M+H] 446.1.

### Example 359

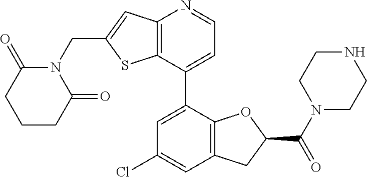 [SMILES: O=C([C@H]1Cc2cc(Cl)cc(-c3ccnc4cc(CN5C(=O)CCCC5=O)sc34)c2O1)N1CCNCC1]

### (R)-1-((7-(5-chloro-2-(piperazine-1-carbonyl)-2,3-dihydrobenzofuran-7-yl)thieno[3,2-b]pyridin-2-yl)methyl)piperidine-2,6-dione

The title compound was synthesized using precursor II, general procedure B and D using 1-((7-chlorothieno[3,2-b]pyridin-2-yl)methyl)piperidine-2,6-dione to give the desired product as the HCl salt. [M+H] 525.0.

### Example 360

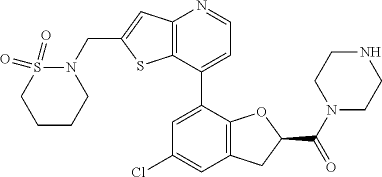 [SMILES: O=C([C@H]1Cc2cc(Cl)cc(-c3ccnc4cc(CN5CCCCS5(=O)=O)sc34)c2O1)N1CCNCC1]

### (R)-(5-chloro-7-(2-((1,1-dioxido-1,2-thiazinan-2-yl)methyl)thieno[3,2-b]pyridin-7-yl)-2,3-dihydrobenzofuran-2-yl)(piperazin-1-yl)methanone

The title compound was synthesized using precursor II, general procedure B and E using 2-((7-chlorothieno[3,2-b]pyridin-2-yl)methyl)-1,2-thiazinane 1,1-dioxide to give the desired product as the TFA salt. [M+H] 547.0.

### Example 361

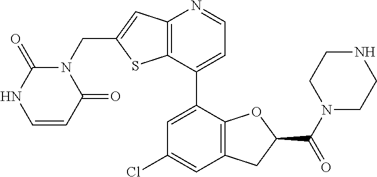 [SMILES: O=C([C@H]1Cc2cc(Cl)cc(-c3ccnc4cc(Cn5c(=O)cc[nH]c5=O)sc34)c2O1)N1CCNCC1]

### (R)-3-((7-(5-chloro-2-(piperazine-1-carbonyl)-2,3-dihydrobenzofuran-7-yl)thieno[3,2-b]pyridin-2-yl)methyl)pyrimidine-2,4(1H,3H)-dione

The title compound was synthesized using precursor II, general procedure B, E and F using 3-((7-chlorothieno[3,2-b]pyridin-2-yl)methyl)pyrimidine-2,4(1H,3H)-dione to give the desired product. [M+H] 524.0.

### Example 362

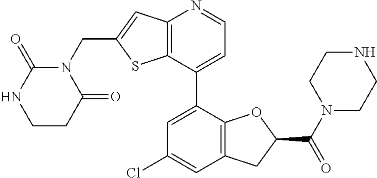 [SMILES: O=C([C@H]1Cc2cc(Cl)cc(-c3ccnc4cc(CN5C(=O)CCNC5=O)sc34)c2O1)N1CCNCC1]

### (R)-3-((7-(5-chloro-2-(piperazine-1-carbonyl)-2,3-dihydrobenzofuran-7-yl)thieno[3,2-b]pyridin-2-yl)methyl)dihydropyrimidine-2,4(1H,3H)-dione

The title compound was synthesized using precursor II, general procedure B, E and F using 3-((7-chlorothieno[3,2-b]pyridin-2-yl)methyl)dihydropyrimidine-2,4(1H,3H)-dione to give the desired product. [M+H] 526.0.

### Example 363

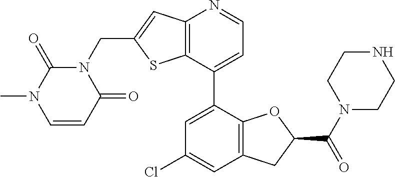 [SMILES: Cn1ccc(=O)n(Cc2cc3nccc(-c4cc(Cl)cc5c4O[C@@H](C(=O)N4CCNCC4)C5)c3s2)c1=O]

### (R)-3-((7-(5-chloro-2-(piperazine-1-carbonyl)-2,3-dihydrobenzofuran-7-yl)thieno[3,2-b]pyridin-2-yl)methyl)-1-methylpyrimidine-2,4(1H,3H)-dione

The title compound was synthesized using precursor II, general procedure B, E and F using 3-((7-chlorothieno[3,2-b]pyridin-2-yl)methyl)-1-methylpyrimidine-2,4(1H,3H)-dione to give the desired product. [M+H] 538.0.

### Example 364

The following compounds were synthesized using one or more of the synthetic methods described earlier herein:

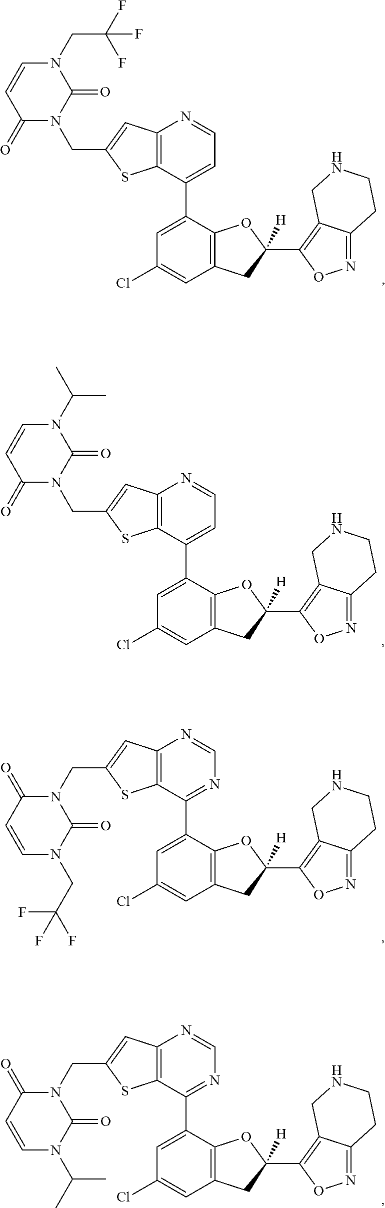 [SMILES: O=c1ccn(CC(F)(F)F)c(=O)n1Cc1cc2nccc(-c3cc(Cl)cc4c3O[C@H](c3onc5c3CNCC5)C4)c2s1 ; CC(C)n1ccc(=O)n(Cc2cc3nccc(-c4cc(Cl)cc5c4O[C@H](c4onc6c4CNCC6)C5)c3s2)c1=O ; O=c1ccn(CC(F)(F)F)c(=O)n1Cc1cc2ncnc(-c3cc(Cl)cc4c3O[C@H](c3onc5c3CNCC5)C4)c2s1 ; CC(C)n1ccc(=O)n(Cc2cc3ncnc(-c4cc(Cl)cc5c4O[C@H](c4onc6c4CNCC6)C5)c3s2)c1=O]

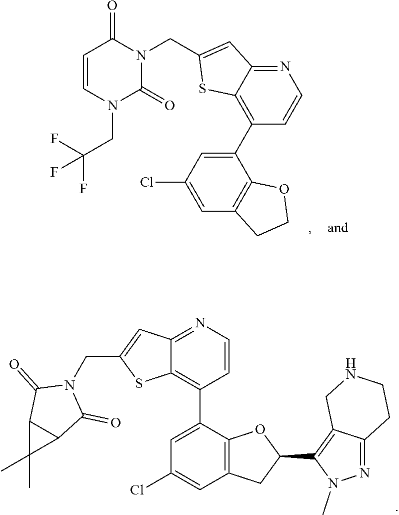 [SMILES: O=c1ccn(CC(F)(F)F)c(=O)n1Cc1cc2nccc(-c3cc(Cl)cc4c3OCC4)c2s1 ; Cn1nc2c(c1[C@H]1Cc3cc(Cl)cc(-c4ccnc5cc(CN6C(=O)C7C(C6=O)C7(C)C)sc45)c3O1)CNCC2]

### BIOLOGICAL EXAMPLES

### Example 365

Cellular Assay

USP7 has been reported to affect the level of polyubiquitination and cellular levels of a number of cellular proteins, including Foxp3 (van Loosdregt, Immunity 2013). As such, the effects of inhibitors of USP7 can be characterized in cellular systems which contain USP7 and its target proteins.

In one example, the potency of USP7 inhibitors can be determined in regulatory T cells (Treg). Treg can be either naturally occurring (nTreg) or induced from naïve T cells (iTreg) following standard protocols such as those described below. These Treg can be derived from either human or murine sources following standard protocols known to those schooled in the art.

CD4⁺ XΔ25⁻ T lymphocytes can be isolated from spleens and/or lymph nodes of mice by magnetic-activated cell sorting. The cells can be cultured in standard culture media [DMEM supplemented with 10% heat inactivated FBS, 1% penicillin streptomycin, 1% non-essential amino acid solution, and mercaptoethanol]. Treg differentiation, if using iTreg, can be induced by culturing these cells for 2-5 days on anti-CD3 antibody coated tissue culture plates with the addition of soluble anti-CD28 antibody, recombinant TGFβ, IL2, and 10 ug/ml each of neutralizing antibodies for IL4 and IFNγ. On or about the third day of stimulation the cells can be re-plated on new tissue culture plates supplemented with recombinant IL2. If using nTreg, cells can be isolated using standard techniques known to those schooled in the art.

After a few days of growth and differentiation (e.g. 5-6 days) the mouse iTreg can be seeded in 96-well plates and the compounds added at various concentrations for a period of time, usually 2-24 hours. Analysis of Foxp3 protein levels can be assessed using standard techniques such as flow cytometry, western blotting or ELISA using antibodies specific for Foxp3. In addition, other endpoints may include looking for up- or down-regulation of Foxp3-regulated gene expression (e.g. CTLA4, IL7r or IL2) and their corresponding proteins using techniques widely known to those schooled in the art. Functional cellular assays can also be used to characterize to the potency of USP7 inhibitors. For example, it is well known that FoxP3 is essential for the ability of Treg to suppress the proliferation of other non-Treg T lymphocytes (Fontenot, et al, Nat Immunol, 2003). As such, the potency USP7 inhibitors may be characterized in assays in which Treg are co-cultured with activated CD4 or CD8 cells following techniques known to those schooled in the art (Collison and Vignali, Methods Mol Biol, 2011). The addition of USP7 inhibitors can occur prior to or during the co-culture with activated CD4 or CD8 T lymphocytes.

USP7 has also been reported to affect the stability of a number of other proteins. Hence, the potency of USP7 inhibitors (e.g., compounds described herein) can be characterized in cellular models in which USP7 and the USP7-regulated proteins coexist. For example, USP7 has been described to regulated the levels of p53 (Li, et al, Nature, 2002), LSD1 (Yi, et al, Oncol Rep. 2016), ASXL1 (Inoue, et al, Leukemia, 2015), Chk1 (Alonso-de Vegal, et al, Cell Cycle, 2014), Tip60 (Dar, et al, Mol Cell Biol, 2013). USP7 inhibitor potency can be assessed by looking at the levels of these target proteins or indirectly, by looking at downstream effects of USP7 inhibition on these or other USP7 substrates. For example, for p53 one can measure p53 protein levels using standard technique or one can measure the impact of p53 stabilization by looking for changes in the expression of p53-regulated genes or their associated proteins (e.g. p21/WAF1/CIP1) (el-Deiry, et al, Cancer Res, 1994).

### Activity

### Example 366

Activity for compounds described herein is provided in Table 1, wherein potency levels are provided as follows: (Potency: USP7 IC₅₀: A<1 μM; B=1-10 μM; C 10-30 μM).

TABLE 1

| Compound | Compound Name | Potency |
|---|---|---|
| 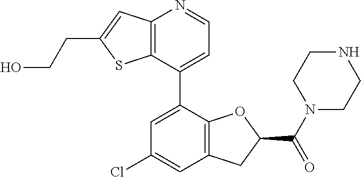 [SMILES: O=C([C@H]1Cc2cc(Cl)cc(-c3ccnc4cc(CCO)sc34)c2O1)N1CCNCC1] | (R)-(5-chloro-7-(2-(2-hydroxyethyl)thieno[3,2- b]pyridin-7-yl)-2,3-dihydrobenzofuran-2- yl)(piperazin-1-yl)methanone | A |
| 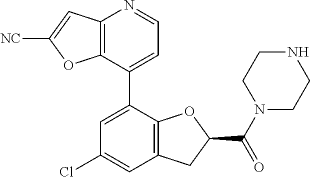 [SMILES: N#Cc1cc2nccc(-c3cc(Cl)cc4c3O[C@@H](C(=O)N3CCNCC3)C4)c2o1] | (R)-7-(5-chloro-2-(piperazine-1-carbonyl)-2,3- dihydrobenzofuran-7-yl)furo[3,2-b]pyridine-2- carbonitrile | B |
| 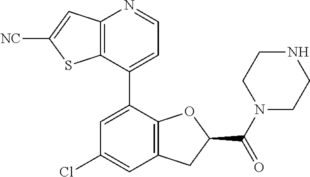 [SMILES: N#Cc1cc2nccc(-c3cc(Cl)cc4c3O[C@@H](C(=O)N3CCNCC3)C4)c2s1] | (R)-7-(5-chloro-2-(piperazine-1-carbonyl)-2,3- dihydrobenzofuran-7-yl)thieno[3,2-b]pyridine- 2-carbonitrile | B |
| 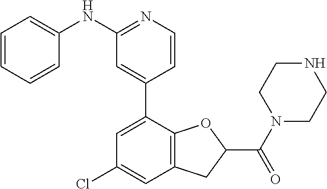 [SMILES: O=C(C1Cc2cc(Cl)cc(-c3ccnc(Nc4ccccc4)c3)c2O1)N1CCNCC1] | (5-chloro-7-(2-(phenylamino)pyridin-4-yl)-2,3- dihydrobenzofuran-2-yl)(piperazin-1- yl)methanone | C |
| 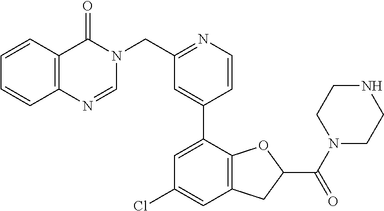 [SMILES: O=C(C1Cc2cc(Cl)cc(-c3ccnc(Cn4cnc5ccccc5c4=O)c3)c2O1)N1CCNCC1] | 3-((4-(5-chloro-2-(piperazine-1-carbonyl)-2,3- dihydrobenzofuran-7-yl)pyridin-2- yl)methyl)quinazolin-4(3H)-one | C |
| 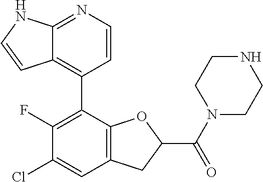 [SMILES: O=C(C1Cc2cc(Cl)c(F)c(-c3ccnc4[nH]ccc34)c2O1)N1CCNCC1] | 5-chloro-6-fluoro-7-(1H-pyrrolo[2,3-b]pyridin- 4-yl)-2,3-dihydrobenzofuran-2-yl)(piperazin-1- yl)methanone | C |
| 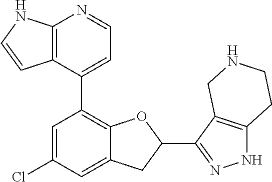 [SMILES: Clc1cc2c(c(-c3ccnc4[nH]ccc34)c1)OC(c1n[nH]c3c1CNCC3)C2] | 3-(5-chloro-7-(1H-pyrrolo[2,3-b]pyridin-4-yl)- 2,3-dihydrobenzofuran-2-yl)-4,5,6,7- tetrahydro-1H-pyrazolo[4,3-c]pyridine | B |
| 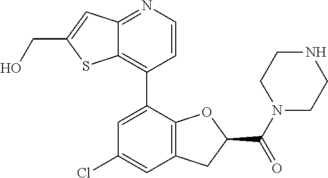 [SMILES: O=C([C@H]1Cc2cc(Cl)cc(-c3ccnc4cc(CO)sc34)c2O1)N1CCNCC1] | (R)-(5-chloro-7-(2-(hydroxymethyl)thieno[3,2- b]pyridin-7-yl)-2,3-dihydrobenzofuran-2- yl)(piperazin-1-yl)methanone | A |
| 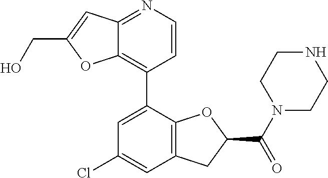 [SMILES: O=C([C@H]1Cc2cc(Cl)cc(-c3ccnc4cc(CO)oc34)c2O1)N1CCNCC1] | (R)-(5-chloro-7-(2-(hydroxymethyl)furo[3,2- b]pyridin-7-yl)-2,3-dihydrobenzofuran-2- yl)(piperazin-1-yl)methanone | A |
| 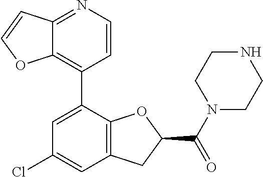 [SMILES: O=C([C@H]1Cc2cc(Cl)cc(-c3ccnc4ccoc34)c2O1)N1CCNCC1] | (R)-(5-chloro-7-(furo[3,2-b]pyridin-7-yl)-2,3- dihydrobenzofuran-2-yl)(piperazin-1- yl)methanone | A |
| 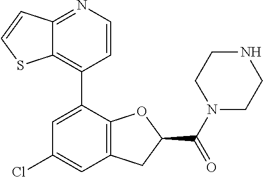 [SMILES: O=C([C@H]1Cc2cc(Cl)cc(-c3ccnc4ccsc34)c2O1)N1CCNCC1] | (R)-(5-chloro-7-(thieno[3,2-b]pyridin-7-yl)-2,3- dihydrobenzofuran-2-yl)(piperazin-1- yl)methanone | A |
| 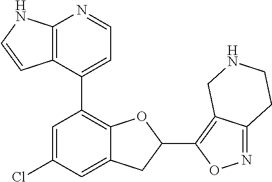 [SMILES: Clc1cc2c(c(-c3ccnc4[nH]ccc34)c1)OC(c1onc3c1CNCC3)C2] | 3-(5-chloro-7-(1H-pyrrolo[2,3-b]pyridin-4-yl)- 2,3-dihydrobenzofuran-2-yl)-4,5,6,7- tetrahydroisoxazolo[4,3-c]pyridine | A |
| 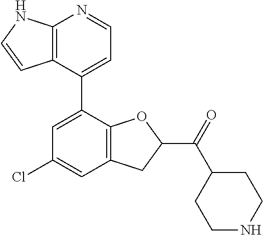 [SMILES: O=C(C1CCNCC1)C1Cc2cc(Cl)cc(-c3ccnc4[nH]ccc34)c2O1] | (5-chloro-7-(1H-pyrrolo[2,3-b]pyridin-4-yl)- 2,3-dihydrobenzofuran-2-yl)(piperidin-4- yl)methanone | B |
| 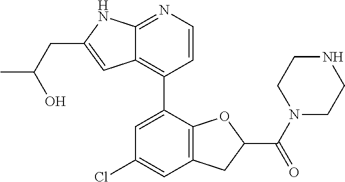 [SMILES: CC(O)Cc1cc2c(-c3cc(Cl)cc4c3OC(C(=O)N3CCNCC3)C4)ccnc2[nH]1] | 5-chloro-7-(2-((S)-2-hydroxypropyl)-1H- pyrrolo[2,3-b]pyridin-4-yl)-2,3- dihydrobenzofuran-2-yl)(piperazin-1- yl)methanone | B |
| 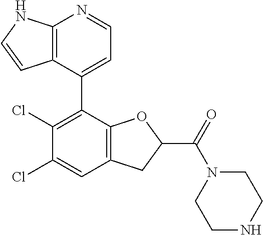 [SMILES: O=C(C1Cc2cc(Cl)c(Cl)c(-c3ccnc4[nH]ccc34)c2O1)N1CCNCC1] | (5,6-dichloro-7-(1H-pyrrolo[2,3-b]pyridin-4- yl)-2,3-dihydrobenzofuran-2-yl)(piperazin-1- yl)methanone | B |
| 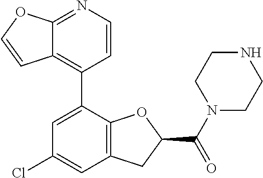 [SMILES: O=C([C@H]1Cc2cc(Cl)cc(-c3ccnc4occc34)c2O1)N1CCNCC1] | (R)-(5-chloro-7-(furo[2,3-b]pyridin-4-yl)-2,3- dihydrobenzofuran-2-yl)(piperazin-1- yl)methanone | B |
| 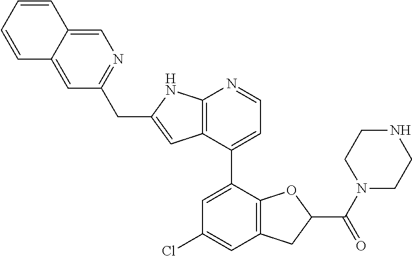 [SMILES: O=C(C1Cc2cc(Cl)cc(-c3ccnc4[nH]c(Cc5cc6ccccc6cn5)cc34)c2O1)N1CCNCC1] | (5-chloro-7-(2-(isoquinolin-3-ylmethyl)-1H- pyrrolo[2,3-b]pyridin-4-yl)-2,3- dihydrobenzofuran-2-yl)(piperazin-1- yl)methanone | B |
| 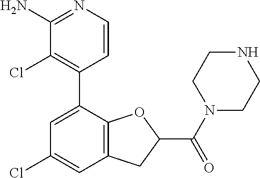 [SMILES: Nc1nccc(-c2cc(Cl)cc3c2OC(C(=O)N2CCNCC2)C3)c1Cl] | (7-(2-amino-3-chloropyridin-4-yl)-5-chloro- 2,3-dihydrobenzofuran-2-yl)(piperazin-1- yl)methanone | B |
| 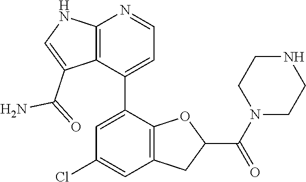 [SMILES: NC(=O)c1c[nH]c2nccc(-c3cc(Cl)cc4c3OC(C(=O)N3CCNCC3)C4)c12] | 4-(5-chloro-2-(piperazine-1-carbonyl)-2,3- dihydrobenzofuran-7-yl)-1H-pyrrolo[2,3- b]pyridine-3-carboxamide | B |
| 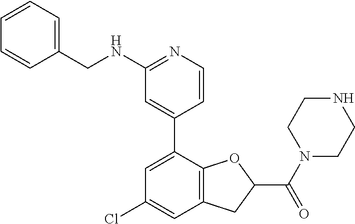 [SMILES: O=C(C1Cc2cc(Cl)cc(-c3ccnc(NCc4ccccc4)c3)c2O1)N1CCNCC1] | (7-(2-(benzylamino)pyridin-4-yl)-5-chloro-2,3- dihydrobenzofuran-2-yl)(piperazin-1- yl)methanone | B |
| 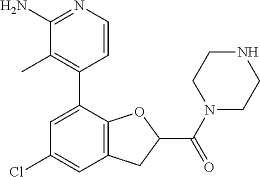 [SMILES: Cc1c(-c2cc(Cl)cc3c2OC(C(=O)N2CCNCC2)C3)ccnc1N] | (7-(2-amino-3-methylpyridin-4-yl)-5-chloro- 2,3-dihydrobenzofuran-2-yl)(piperazin-1- yl)methanone | B |
| 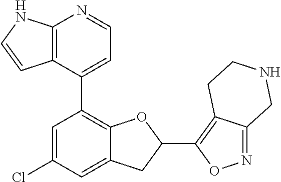 [SMILES: Clc1cc2c(c(-c3ccnc4[nH]ccc34)c1)OC(c1onc3c1CCNC3)C2] | 3-(5-chloro-7-(1H-pyrrolo[2,3-b]pyridin-4-yl)- 2,3-dihydrobenzofuran-2-yl)-4,5,6,7- tetrahydroisoxazolo[3,4-c]pyridine | B |
| 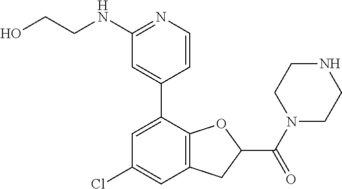 [SMILES: O=C(C1Cc2cc(Cl)cc(-c3ccnc(NCCO)c3)c2O1)N1CCNCC1] | (5-chloro-7-(2-((2- hydroxyethyl)amino)pyridin-4-yl)-2,3- dihydrobenzofuran-2-yl)(piperazin-1- yl)methanone | B |
| 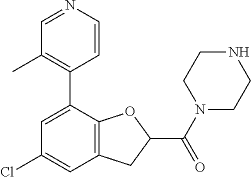 [SMILES: Cc1cnccc1-c1cc(Cl)cc2c1OC(C(=O)N1CCNCC1)C2] | 5-chloro-7-(3-methylpyridin-4-yl)-2,3- dihydrobenzofuran-2-yl)(piperazin-1- yl)methanone | C |
| 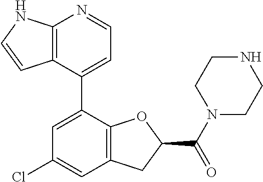 [SMILES: O=C([C@H]1Cc2cc(Cl)cc(-c3ccnc4[nH]ccc34)c2O1)N1CCNCC1] | (R)-(5-chloro-7-(1H-pyrrolo[2,3-b]pyridin-4- yl)-2,3-dihydrobenzofuran-2-yl)(piperazin-1- yl)methanone | A |
| 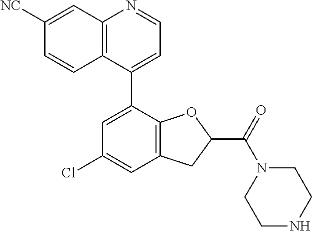 [SMILES: N#Cc1ccc2c(-c3cc(Cl)cc4c3OC(C(=O)N3CCNCC3)C4)ccnc2c1] | 4-(5-chloro-2-(piperazine-1-carbonyl)-2,3- dihydrobenzofuran-7-yl)quinoline-7- carbonitrile | A |
| 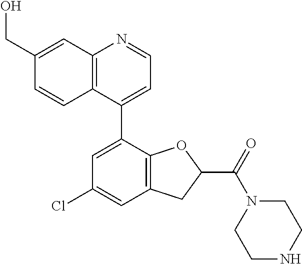 [SMILES: O=C(C1Cc2cc(Cl)cc(-c3ccnc4cc(CO)ccc34)c2O1)N1CCNCC1] | (5-chloro-7-(7-(hydroxymethyl)quinolin-4-yl)- 2,3-dihydrobenzofuran-2-yl)(piperazin-1- yl)methanone | B |
| 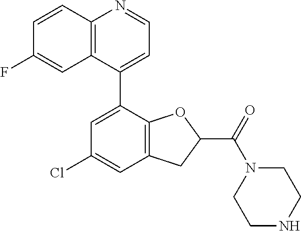 [SMILES: O=C(C1Cc2cc(Cl)cc(-c3ccnc4ccc(F)cc34)c2O1)N1CCNCC1] | (5-chloro-7-(6-fluoroquinolin-4-yl)-2,3- dihydrobenzofuran-2-yl)(piperazin-1- yl)methanone | B |
| 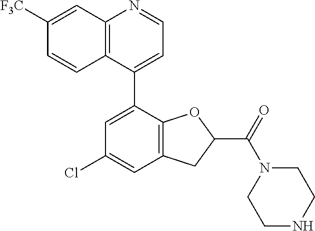 [SMILES: O=C(C1Cc2cc(Cl)cc(-c3ccnc4cc(C(F)(F)F)ccc34)c2O1)N1CCNCC1] | (5-chloro-7-(7-(trifluoromethyl)quinolin-4-yl)- 2,3-dihydrobenzofuran-2-yl)(piperazin-1- yl)methanone | B |
| 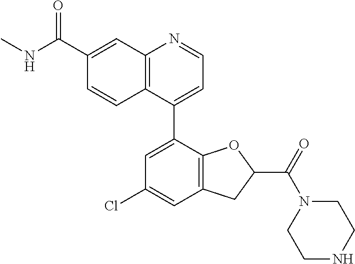 [SMILES: CNC(=O)c1ccc2c(-c3cc(Cl)cc4c3OC(C(=O)N3CCNCC3)C4)ccnc2c1] | 4-(5-chloro-2-(piperazine-1-carbonyl)-2,3- dihydrobenzofuran-7-yl)-N-methylquinoline-7- carboxamide | B |
| 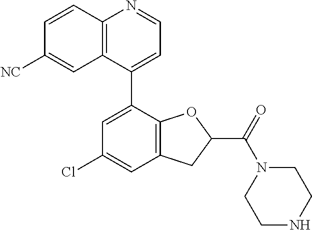 [SMILES: N#Cc1ccc2nccc(-c3cc(Cl)cc4c3OC(C(=O)N3CCNCC3)C4)c2c1] | 4-(5-chloro-2-(piperazine-1-carbonyl)-2,3- dihydrobenzofuran-7-yl)quinoline-6- carbonitrile | C |
| 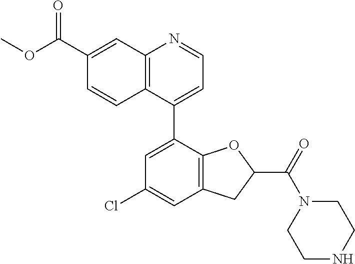 [SMILES: COC(=O)c1ccc2c(-c3cc(Cl)cc4c3OC(C(=O)N3CCNCC3)C4)ccnc2c1] | methyl 4-(5-chloro-2-(piperazine-1-carbonyl)- 2,3-dihydrobenzofuran-7-yl)quinoline-7- carboxylate | C |
| 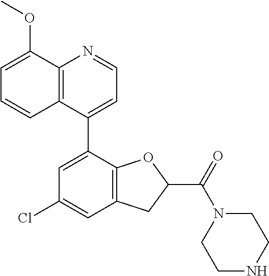 [SMILES: COc1cccc2c(-c3cc(Cl)cc4c3OC(C(=O)N3CCNCC3)C4)ccnc12] | (5-chloro-7-(8-methoxyquinolin-4-yl)-2,3- dihydrobenzofuran-2-yl)(piperazin-1- yl)methanone | C |
| 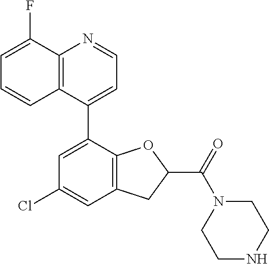 [SMILES: O=C(C1Cc2cc(Cl)cc(-c3ccnc4c(F)cccc34)c2O1)N1CCNCC1] | (5-chloro-7-(8-fluoroquinolin-4-yl)-2,3- dihydrobenzofuran-2-yl)(piperazin-1- yl)methanone | C |
| 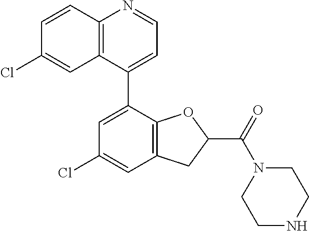 [SMILES: O=C(C1Cc2cc(Cl)cc(-c3ccnc4ccc(Cl)cc34)c2O1)N1CCNCC1] | (5-chloro-7-(6-chloroquinolin-4-yl)-2,3- dihydrobenzofuran-2-yl)(piperazin-1- yl)methanone | C |
| 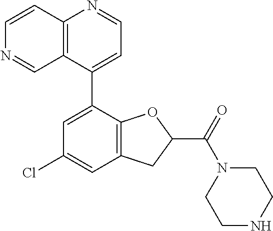 [SMILES: O=C(C1Cc2cc(Cl)cc(-c3ccnc4ccncc34)c2O1)N1CCNCC1] | (5-chloro-7-(1,6-naphthyridin-4-yl)-2,3- dihydrobenzofuran-2-yl)(piperazin-1- yl)methanone | C |
| 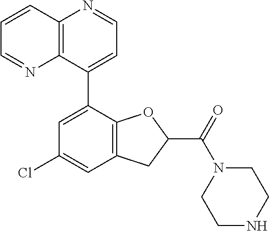 [SMILES: O=C(C1Cc2cc(Cl)cc(-c3ccnc4cccnc34)c2O1)N1CCNCC1] | (5-chloro-7-(1,5-naphthyridin-4-yl)-2,3- dihydrobenzofuran-2-yl)(piperazin-1- yl)methanone | C |
| 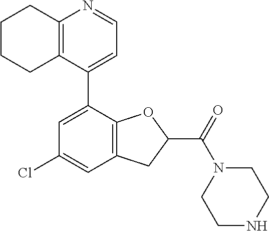 [SMILES: O=C(C1Cc2cc(Cl)cc(-c3ccnc4c3CCCC4)c2O1)N1CCNCC1] | (5-chloro-7-(5,6,7,8-tetrahydroquinolin-4-yl)- 2,3-dihydrobenzofuran-2-yl)(piperazin-1- yl)methanone | A |
| 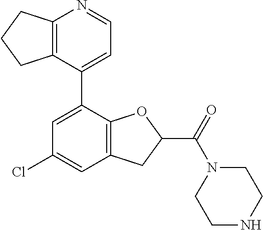 [SMILES: O=C(C1Cc2cc(Cl)cc(-c3ccnc4c3CCC4)c2O1)N1CCNCC1] | (5-chloro-7-(6,7-dihydro-5H- cyclopenta[b]pyridin-4-yl)-2,3- dihydrobenzofuran-2-yl)(piperazin-1- yl)methanone | A |
| 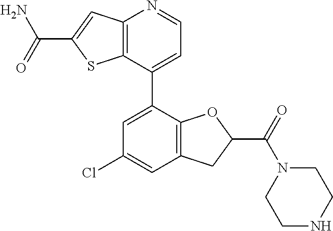 [SMILES: NC(=O)c1cc2nccc(-c3cc(Cl)cc4c3OC(C(=O)N3CCNCC3)C4)c2s1] | 7-(5-chloro-2-(piperazine-1-carbonyl)-2,3- dihydrobenzofuran-7-yl)thieno[3,2-b]pyridine- 2-carboxamide | B |
| 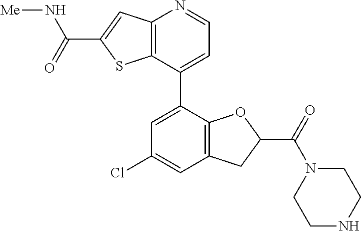 [SMILES: CNC(=O)c1cc2nccc(-c3cc(Cl)cc4c3OC(C(=O)N3CCNCC3)C4)c2s1] | 7-(5-chloro-2-(piperazine-1-carbonyl)-2,3- dihydrobenzofuran-7-yl)thieno[3,2-b]pyridine- 2-carboxamide | B |
| 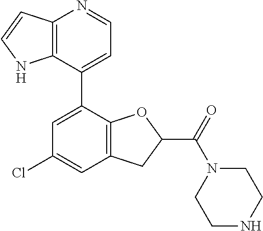 [SMILES: O=C(C1Cc2cc(Cl)cc(-c3ccnc4cc[nH]c34)c2O1)N1CCNCC1] | (5-chloro-7-(1H-pyrrolo[3,2-b]pyridin-7-yl)- 2,3-dihydrobenzofuran-2-yl)(piperazin-1- yl)methanone | B |
| 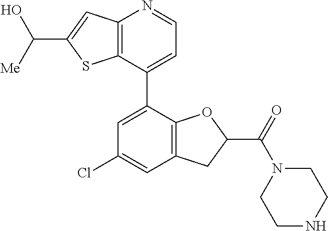 [SMILES: CC(O)c1cc2nccc(-c3cc(Cl)cc4c3OC(C(=O)N3CCNCC3)C4)c2s1] | ((2R)-5-chloro-7-(2-(1- hydroxyethyl)thieno[3,2-b]pyridin-7-yl)-2,3- dihydrobenzofuran-2-yl)(piperazin-1- yl)methanone | A |
| 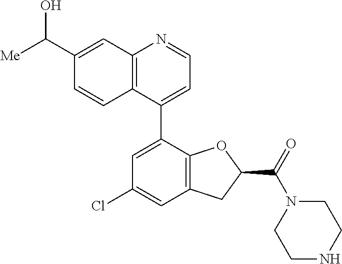 [SMILES: CC(O)c1ccc2c(-c3cc(Cl)cc4c3O[C@@H](C(=O)N3CCNCC3)C4)ccnc2c1] | ((2R)-5-chloro-7-(7-(1-hydroxyethyl)quinolin- 4-yl)-2,3-dihydrobenzofuran-2-yl)(piperazin-1- yl)methanone | A |
| 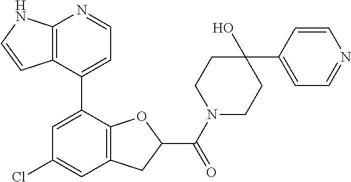 [SMILES: O=C(C1Cc2cc(Cl)cc(-c3ccnc4[nH]ccc34)c2O1)N1CCC(O)(c2ccncc2)CC1] | (5-chloro-7-(1H-pyrrolo[2,3-b]pyridin-4-yl)- 2,3-dihydrobenzofuran-2-yl)(4-hydroxy-4- (pyridin-4-yl)piperidin-1-yl)methanone | A |
| 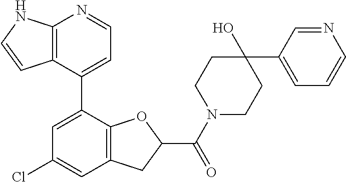 [SMILES: O=C(C1Cc2cc(Cl)cc(-c3ccnc4[nH]ccc34)c2O1)N1CCC(O)(c2cccnc2)CC1] | (5-chloro-7-(1H-pyrrolo[2,3-b]pyridin-4-yl)- 2,3-dihydrobenzofuran-2-yl)(4-hydroxy-4- (pyridin-3-yl)piperidin-1-yl)methanone | A |
| 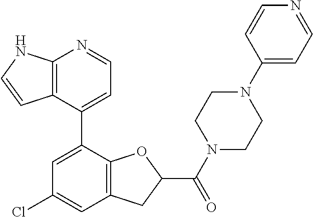 [SMILES: O=C(C1Cc2cc(Cl)cc(-c3ccnc4[nH]ccc34)c2O1)N1CCN(c2ccncc2)CC1] | (5-chloro-7-(1H-pyrrolo[2,3-b]pyridin-4-yl)- 2,3-dihydrobenzofuran-2-yl)(4-(pyridin-4- yl)piperazin-1-yl)methanone | B |
| 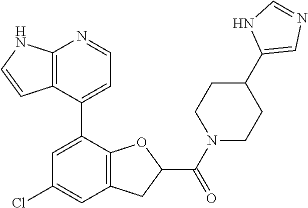 [SMILES: O=C(C1Cc2cc(Cl)cc(-c3ccnc4[nH]ccc34)c2O1)N1CCC(c2cnc[nH]2)CC1] | (4-(1H-imidazol-5-yl)piperidin-1-yl)(5-chloro- 7-(1H-pyrrolo[2,3-b]pyridin-4-yl)-2,3- dihydrobenzofuran-2-yl)methanone | B |
| 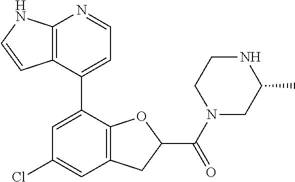 [SMILES: C[C@@H]1CN(C(=O)C2Cc3cc(Cl)cc(-c4ccnc5[nH]ccc45)c3O2)CCN1] | (5-chloro-7-(1H-pyrrolo[2,3-b]pyridin-4-yl)- 2,3-dihydrobenzofuran-2-yl)((R)-3- methylpiperazin-1-yl)methanone | B |
| 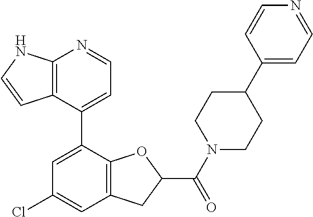 [SMILES: O=C(C1Cc2cc(Cl)cc(-c3ccnc4[nH]ccc34)c2O1)N1CCC(c2ccncc2)CC1] | (5-chloro-7-(1H-pyrrolo[2,3-b]pyridin-4-yl)- 2,3-dihydrobenzofuran-2-yl)(4-(pyridin-4- yl)piperidin-1-yl)methanone | B |
| 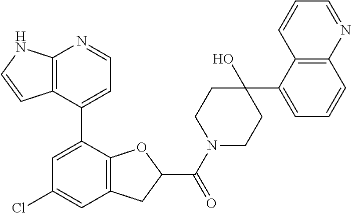 [SMILES: O=C(C1Cc2cc(Cl)cc(-c3ccnc4[nH]ccc34)c2O1)N1CCC(O)(c2cccc3ncccc23)CC1] | (5-chloro-7-(1H-pyrrolo[2,3-b]pyridin-4-yl)- 2,3-dihydrobenzofuran-2-yl)(4-hydroxy-4- (quinolin-5-yl)piperidin-1-yl)methanone | B |
| 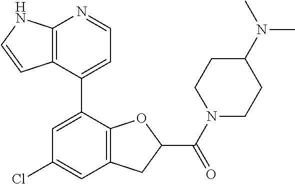 [SMILES: CN(C)C1CCN(C(=O)C2Cc3cc(Cl)cc(-c4ccnc5[nH]ccc45)c3O2)CC1] | (5-chloro-7-(1H-pyrrolo[2,3-b]pyridin-4-yl)- 2,3-dihydrobenzofuran-2-yl)(4- (dimethylamino)piperidin-1-yl)methanone | B |
| 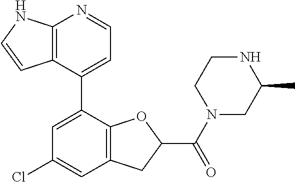 [SMILES: C[C@H]1CN(C(=O)C2Cc3cc(Cl)cc(-c4ccnc5[nH]ccc45)c3O2)CCN1] | (5-chloro-7-(1H-pyrrolo[2,3-b]pyridin-4-yl)- 2,3-dihydrobenzofuran-2-yl)((S)-3- methylpiperazin-1-yl)methanone | B |
| 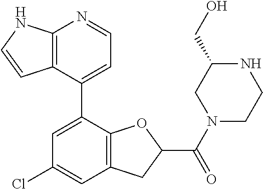 [SMILES: O=C(C1Cc2cc(Cl)cc(-c3ccnc4[nH]ccc34)c2O1)N1CCN[C@@H](CO)C1] | (5-chloro-7-(1H-pyrrolo[2,3-b]pyridin-4-yl)- 2,3-dihydrobenzofuran-2-yl)((R)-3- (hydroxymethyl)piperazin-1-yl)methanone | B |
| 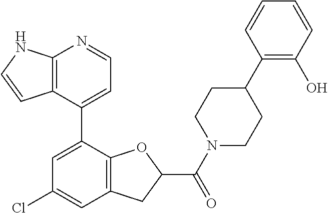 [SMILES: O=C(C1Cc2cc(Cl)cc(-c3ccnc4[nH]ccc34)c2O1)N1CCC(c2ccccc2O)CC1] | (5-chloro-7-(1H-pyrrolo[2,3-b]pyridin-4-yl)- 2,3-dihydrobenzofuran-2-yl)(4-(2- hydroxyphenyl)piperidin-1-yl)methanone | B |
| 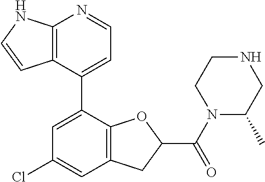 [SMILES: C[C@H]1CNCCN1C(=O)C1Cc2cc(Cl)cc(-c3ccnc4[nH]ccc34)c2O1] | (5-chloro-7-(1H-pyrrolo[2,3-b]pyridin-4-yl)- 2,3-dihydrobenzofuran-2-yl)((S)-2- methylpiperazin-1-yl)methanone | B |
| 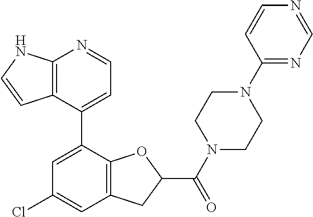 [SMILES: O=C(C1Cc2cc(Cl)cc(-c3ccnc4[nH]ccc34)c2O1)N1CCN(c2ccncn2)CC1] | (5-chloro-7-(1H-pyrrolo[2,3-b]pyridin-4-yl)- 2,3-dihydrobenzofuran-2-yl)(4-(pyrimidin-4- yl)piperazin-1-yl)methanone | B |
| 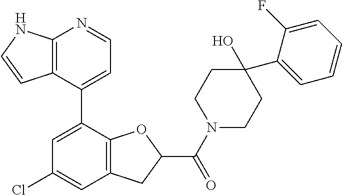 [SMILES: O=C(C1Cc2cc(Cl)cc(-c3ccnc4[nH]ccc34)c2O1)N1CCC(O)(c2ccccc2F)CC1] | (5-chloro-7-(1H-pyrrolo[2,3-b]pyridin-4-yl)- 2,3-dihydrobenzofuran-2-yl)(4-(2- fluorophenyl)-4-hydroxypiperidin-1- yl)methanone | B |
| 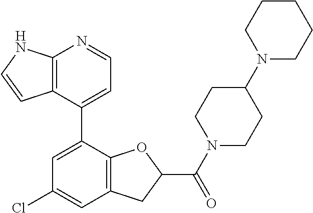 [SMILES: O=C(C1Cc2cc(Cl)cc(-c3ccnc4[nH]ccc34)c2O1)N1CCC(N2CCCCC2)CC1] | 1,4′-bipiperidin-1′-yl(5-chloro-7-(1H- pyrrolo[2,3-b]pyridin-4-yl)-2,3- dihydrobenzofuran-2-yl)methanone | B |
| 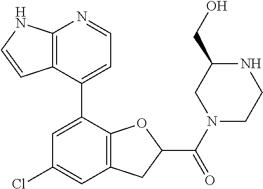 [SMILES: O=C(C1Cc2cc(Cl)cc(-c3ccnc4[nH]ccc34)c2O1)N1CCN[C@H](CO)C1] | (5-chloro-7-(1H-pyrrolo[2,3-b]pyridin-4-yl)- 2,3-dihydrobenzofuran-2-yl)((S)-3- (hydroxymethyl)piperazin-1-yl)methanone | B |
| 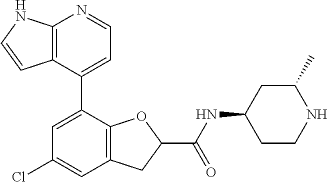 [SMILES: C[C@H]1C[C@H](NC(=O)C2Cc3cc(Cl)cc(-c4ccnc5[nH]ccc45)c3O2)CCN1] | 5-chloro-N-((2S,4R)-2-methylpiperidin-4-yl)-7- (1H-pyrrolo[2,3-b]pyridin-4-yl)-2,3- dihydrobenzofuran-2-carboxamide | B |
| 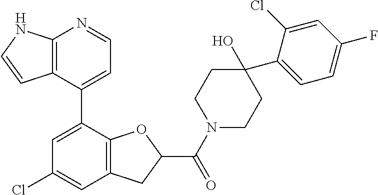 [SMILES: O=C(C1Cc2cc(Cl)cc(-c3ccnc4[nH]ccc34)c2O1)N1CCC(O)(c2ccc(F)cc2Cl)CC1] | (4-(2-chloro-4-fluorophenyl)-4- hydroxypiperidin-1-yl)(5-chloro-7-(1H- pyrrolo[2,3-b]pyridin-4-yl)-2,3- dihydrobenzofuran-2-yl)methanone | B |
| 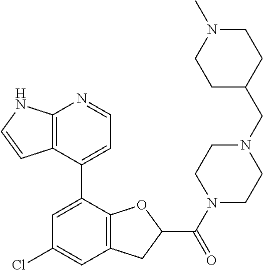 [SMILES: CN1CCC(CN2CCN(C(=O)C3Cc4cc(Cl)cc(-c5ccnc6[nH]ccc56)c4O3)CC2)CC1] | (5-chloro-7-(1H-pyrrolo[2,3-b]pyridin-4-yl)- 2,3-dihydrobenzofuran-2-yl)(4-((1- methylpiperidin-4-yl)methyl)piperazin-1- yl)methanone | B |
| 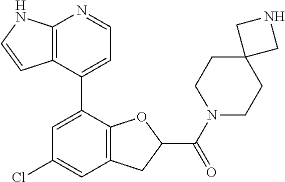 [SMILES: O=C(C1Cc2cc(Cl)cc(-c3ccnc4[nH]ccc34)c2O1)N1CCC2(CC1)CNC2] | (5-chloro-7-(1H-pyrrolo[2,3-b]pyridin-4-yl)- 2,3-dihydrobenzofuran-2-yl)(2,7- diazaspiro[3.5]nonan-7-yl)methanone | B |
| 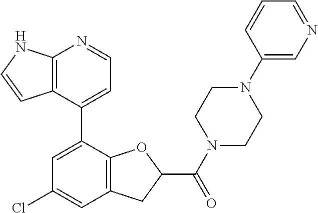 [SMILES: O=C(C1Cc2cc(Cl)cc(-c3ccnc4[nH]ccc34)c2O1)N1CCN(c2cccnc2)CC1] | (5-chloro-7-(1H-pyrrolo[2,3-b]pyridin-4-yl)- 2,3-dihydrobenzofuran-2-yl)(4-(pyridin-3- yl)piperazin-1-yl)methanone | B |
| 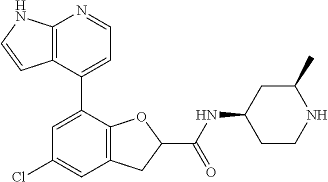 [SMILES: C[C@@H]1C[C@H](NC(=O)C2Cc3cc(Cl)cc(-c4ccnc5[nH]ccc45)c3O2)CCN1] | 5-chloro-N-((2R,4R)-2-methylpiperidin-4-yl)-7- (1H-pyrrolo[2,3-b]pyridin-4-yl)-2,3- dihydrobenzofuran-2-carboxamide | B |
| 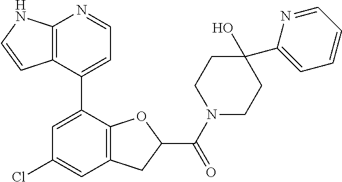 [SMILES: O=C(C1Cc2cc(Cl)cc(-c3ccnc4[nH]ccc34)c2O1)N1CCC(O)(c2ccccn2)CC1] | (5-chloro-7-(1H-pyrrolo[2,3-b]pyridin-4-yl)- 2,3-dihydrobenzofuran-2-yl)(4-hydroxy-4- (pyridin-2-yl)piperidin-1-yl)methanone | B |
| 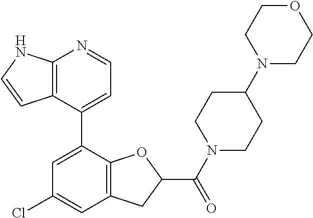 [SMILES: O=C(C1Cc2cc(Cl)cc(-c3ccnc4[nH]ccc34)c2O1)N1CCC(N2CCOCC2)CC1] | (5-chloro-7-(1H-pyrrolo[2,3-b]pyridin-4-yl)- 2,3-dihydrobenzofuran-2-yl)(4- morpholinopiperidin-1-yl)methanone | B |
| 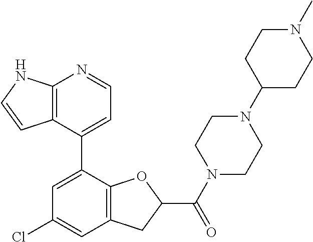 [SMILES: CN1CCC(N2CCN(C(=O)C3Cc4cc(Cl)cc(-c5ccnc6[nH]ccc56)c4O3)CC2)CC1] | (5-chloro-7-(1H-pyrrolo[2,3-b]pyridin-4-yl)- 2,3-dihydrobenzofuran-2-yl)(4-(1- methylpiperidin-4-yl)piperazin-1-yl)methanone | C |
| 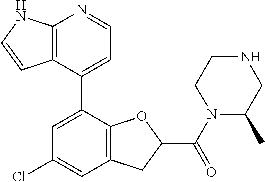 [SMILES: C[C@@H]1CNCCN1C(=O)C1Cc2cc(Cl)cc(-c3ccnc4[nH]ccc34)c2O1] | (5-chloro-7-(1H-pyrrolo[2,3-b]pyridin-4-yl)- 2,3-dihydrobenzofuran-2-yl)((R)-2- methylpiperazin-1-yl)methanone | C |
| 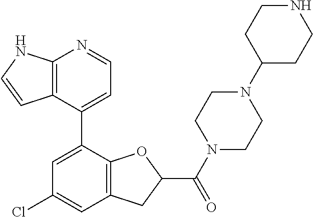 [SMILES: O=C(C1Cc2cc(Cl)cc(-c3ccnc4[nH]ccc34)c2O1)N1CCN(C2CCNCC2)CC1] | (5-chloro-7-(1H-pyrrolo[2,3-b]pyridin-4-yl)- 2,3-dihydrobenzofuran-2-yl)(4-(piperidin-4- yl)piperazin-1-yl)methanone | C |
| 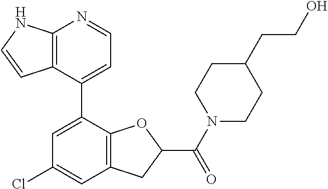 [SMILES: O=C(C1Cc2cc(Cl)cc(-c3ccnc4[nH]ccc34)c2O1)N1CCC(CCO)CC1] | (R)-(5-chloro-7-(1H-pyrrolo[2,3-b]pyridin-4- yl)-2,3-dihydrobenzofuran-2-yl)(4-(2- hydroxyethyl)piperidin-1-yl)methanone | C |
| 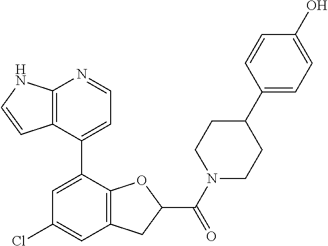 [SMILES: O=C(C1Cc2cc(Cl)cc(-c3ccnc4[nH]ccc34)c2O1)N1CCC(c2ccc(O)cc2)CC1] | (5-chloro-7-(1H-pyrrolo[2,3-b]pyridin-4-yl)- 2,3-dihydrobenzofuran-2-yl)(4-(4- hydroxyphenyl)piperidin-1-yl)methanone | C |
| 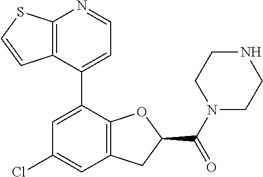 [SMILES: O=C([C@H]1Cc2cc(Cl)cc(-c3ccnc4sccc34)c2O1)N1CCNCC1] | (R)-(5-chloro-7-(thieno[2,3-b]pyridin-4-yl)-2,3- dihydrobenzofuran-2-yl)(piperazin-1- yl)methanone | B |
| 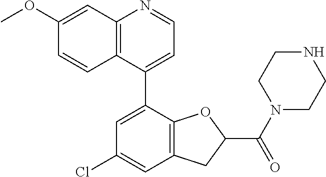 [SMILES: COc1ccc2c(-c3cc(Cl)cc4c3OC(C(=O)N3CCNCC3)C4)ccnc2c1] | (5-chloro-7-(7-methoxyquinolin-4-yl)-2,3- dihydrobenzofuran-2-yl)(piperazin-1- yl)methanone | B |
| 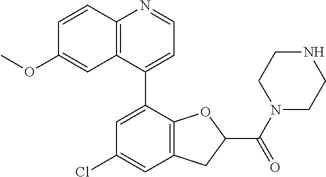 [SMILES: COc1ccc2nccc(-c3cc(Cl)cc4c3OC(C(=O)N3CCNCC3)C4)c2c1] | (5-chloro-7-(6-methoxyquinolin-4-yl)-2,3- dihydrobenzofuran-2-yl)(piperazin-1- yl)methanone | C |
| 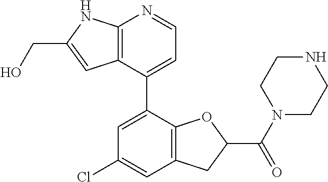 [SMILES: O=C(C1Cc2cc(Cl)cc(-c3ccnc4[nH]c(CO)cc34)c2O1)N1CCNCC1] | (5-chloro-7-(2-(hydroxymethyl)-1H- pyrrolo[2,3-b]pyridin-4-yl)-2,3- dihydrobenzofuran-2-yl)(piperazin-1- yl)methanone | A |
| 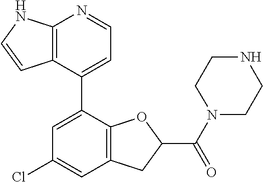 [SMILES: O=C(C1Cc2cc(Cl)cc(-c3ccnc4[nH]ccc34)c2O1)N1CCNCC1] | (5-chloro-7-(1H-pyrrolo[2,3-b]pyridin-4-yl)- 2,3-dihydrobenzofuran-2-yl)(piperazin-1- yl)methanone | A |
| 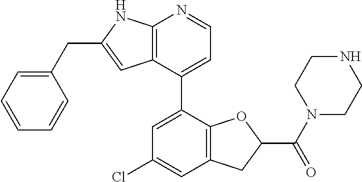 [SMILES: O=C(C1Cc2cc(Cl)cc(-c3ccnc4[nH]c(Cc5ccccc5)cc34)c2O1)N1CCNCC1] | (7-(2-benzyl-1H-pyrrolo[2,3-b]pyridin-4-yl)-5- chloro-2,3-dihydrobenzofuran-2-yl)(piperazin- 1-yl)methanone | B |
| 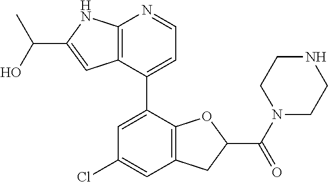 [SMILES: CC(O)c1cc2c(-c3cc(Cl)cc4c3OC(C(=O)N3CCNCC3)C4)ccnc2[nH]1] | (5-chloro-7-(2-((S)-1-hydroxyethyl)-1H- pyrrolo[2,3-b]pyridin-4-yl)-2,3- dihydrobenzofuran-2-yl)(piperazin-1- yl)methanone | B |
| 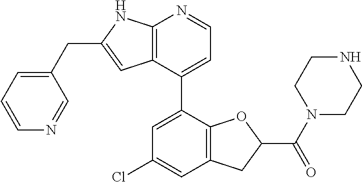 [SMILES: O=C(C1Cc2cc(Cl)cc(-c3ccnc4[nH]c(Cc5cccnc5)cc34)c2O1)N1CCNCC1] | (5-chloro-7-(2-(pyridin-3-ylmethyl)-1H- pyrrolo[2,3-b]pyridin-4-yl)-2,3- dihydrobenzofuran-2-yl)(piperazin-1- yl)methanone | B |
| 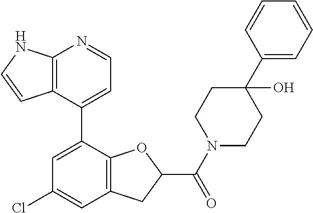 [SMILES: O=C(C1Cc2cc(Cl)cc(-c3ccnc4[nH]ccc34)c2O1)N1CCC(O)(c2ccccc2)CC1] | (5-chloro-7-(1H-pyrrolo[2,3-b]pyridin-4-yl)- 2,3-dihydrobenzofuran-2-yl)(4-hydroxy-4- phenylpiperidin-1-yl)methanone | B |
| 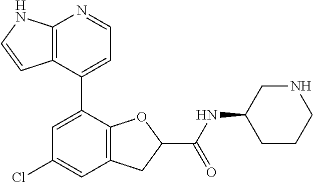 [SMILES: O=C(N[C@@H]1CCCNC1)C1Cc2cc(Cl)cc(-c3ccnc4[nH]ccc34)c2O1] | 5-Chloro-N-((R)-piperidin-3-yl)-7-(1H- pyrrolo[2,3-b]pyridin-4-yl)-2,3- dihydrobenzofuran-2-carboxamide | B |
| 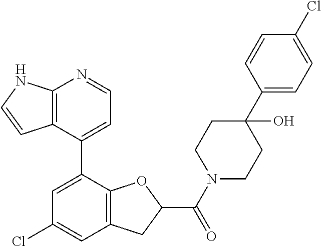 [SMILES: O=C(C1Cc2cc(Cl)cc(-c3ccnc4[nH]ccc34)c2O1)N1CCC(O)(c2ccc(Cl)cc2)CC1] | (5-chloro-7-(1H-pyrrolo[2,3-b]pyridin-4-yl)- 2,3-dihydrobenzofuran-2-yl)(4-(4- chlorophenyl)-4-hydroxypiperidin-1- yl)methanone | B |
| 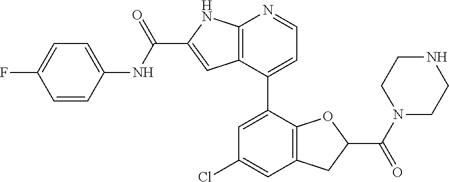 [SMILES: O=C(Nc1ccc(F)cc1)c1cc2c(-c3cc(Cl)cc4c3OC(C(=O)N3CCNCC3)C4)ccnc2[nH]1] | 4-(5-chloro-2-(piperazine-1-carbonyl)-2,3- dihydrobenzofuran-7-yl)-N-(4-fluorophenyl)- 1H-pyrrolo[2,3-b]pyridine-2-carboxamide | B |
| 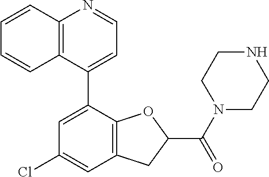 [SMILES: O=C(C1Cc2cc(Cl)cc(-c3ccnc4ccccc34)c2O1)N1CCNCC1] | (5-chloro-7-(quinolin-4-yl)-2,3- dihydrobenzofuran-2-yl)(piperazin-1- yl)methanone | B |
| 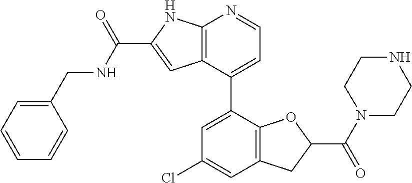 [SMILES: O=C(NCc1ccccc1)c1cc2c(-c3cc(Cl)cc4c3OC(C(=O)N3CCNCC3)C4)ccnc2[nH]1] | N-benzyl-4-(5-chloro-2-(piperazine-1- carbonyl)-2,3-dihydrobenzofuran-7-yl)-1H- pyrrolo[2,3-b]pyridine-2-carboxamide | B |
| 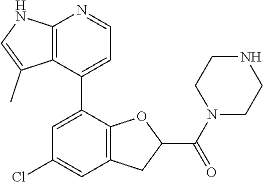 [SMILES: Cc1c[nH]c2nccc(-c3cc(Cl)cc4c3OC(C(=O)N3CCNCC3)C4)c12] | (5-chloro-7-(3-methyl-1H-pyrrolo[2,3- b]pyridin-4-yl)-2,3-dihydrobenzofuran-2- yl)(piperazin-1-yl)methanone | B |
| 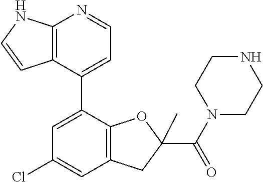 [SMILES: CC1(C(=O)N2CCNCC2)Cc2cc(Cl)cc(-c3ccnc4[nH]ccc34)c2O1] | (5-chloro-2-methyl-7-(1H-pyrrolo[2,3- b]pyridin-4-yl)-2,3-dihydrobenzofuran-2- yl)(piperazin-1-yl)methanone | B |
| 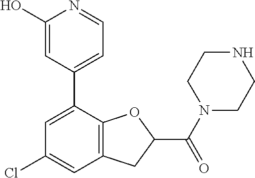 [SMILES: O=C(C1Cc2cc(Cl)cc(-c3ccnc(O)c3)c2O1)N1CCNCC1] | (5-chloro-7-(2-hydroxypyridin-4-yl)-2,3- dihydrobenzofuran-2-yl)(piperazin-1- yl)methanone | B |
| 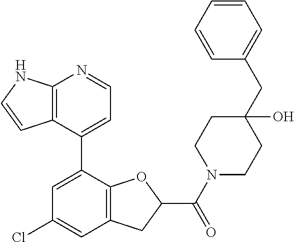 [SMILES: O=C(C1Cc2cc(Cl)cc(-c3ccnc4[nH]ccc34)c2O1)N1CCC(O)(Cc2ccccc2)CC1] | (4-benzyl-4-hydroxypiperidin-1-yl)(5-chloro-7- (1H-pyrrolo[2,3-b]pyridin-4-yl)-2,3- dihydrobenzofuran-2-yl)methanone | B |
| 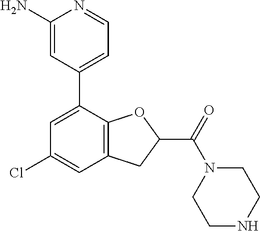 [SMILES: Nc1cc(-c2cc(Cl)cc3c2OC(C(=O)N2CCNCC2)C3)ccn1] | (7-(2-Aminopyridin-4-yl)-5-chloro-2,3- dihydrobenzofuran-2-yl)(piperazin-1- yl)methanone | B |
| 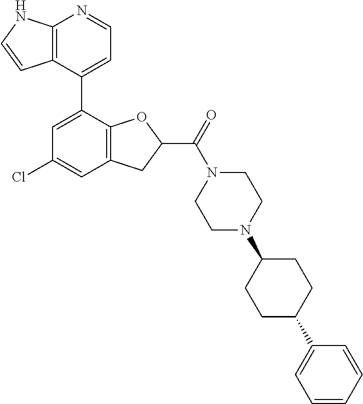 [SMILES: O=C(C1Cc2cc(Cl)cc(-c3ccnc4[nH]ccc34)c2O1)N1CCN([C@H]2CC[C@H](c3ccccc3)CC2)CC1] | (5-chloro-7-(1H-pyrrolo[2,3-b]pyridin-4-yl)- 2,3-dihydrobenzofuran-2-yl)(4-((1s,4r)-4- phenylcyclohexyl)piperazin-1-yl)methanone | B |
| 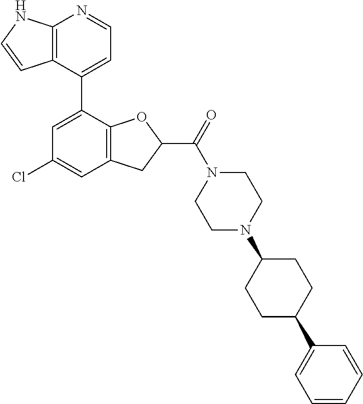 [SMILES: O=C(C1Cc2cc(Cl)cc(-c3ccnc4[nH]ccc34)c2O1)N1CCN([C@H]2CC[C@@H](c3ccccc3)CC2)CC1] | (5-chloro-7-(1H-pyrrolo[2,3-b]pyridin-4-yl)- 2,3-dihydrobenzofuran-2-yl)(4-((1s,4s)-4- phenylcyclohexyl)piperazin-1-yl)methanone | B |
| 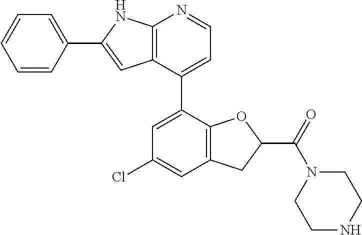 [SMILES: O=C(C1Cc2cc(Cl)cc(-c3ccnc4[nH]c(-c5ccccc5)cc34)c2O1)N1CCNCC1] | (5-chloro-7-(2-phenyl-1H-pyrrolo[2,3- b]pyridin-4-yl)-2,3-dihydrobenzofuran-2- yl)(piperazin-1-yl)methanone | C |
| 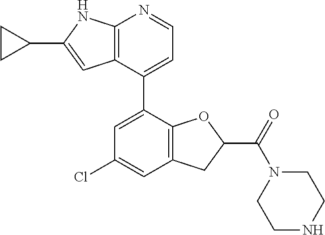 [SMILES: O=C(C1Cc2cc(Cl)cc(-c3ccnc4[nH]c(C5CC5)cc34)c2O1)N1CCNCC1] | (5-chloro-7-(2-cyclopropyl-1H-pyrrolo[2,3- b]pyridin-4-yl)-2,3-dihydrobenzofuran-2- yl)(piperazin-1-yl)methanone | C |
| 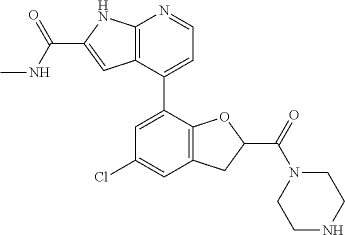 [SMILES: CNC(=O)c1cc2c(-c3cc(Cl)cc4c3OC(C(=O)N3CCNCC3)C4)ccnc2[nH]1] | 4-(5-Chloro-2-(piperazine-1-carbonyl)-2,3- dihydrobenzofuran-7-yl)-N-methyl-1H- pyrrolo[2,3-b]pyridine-2-carboxamide | C |
| 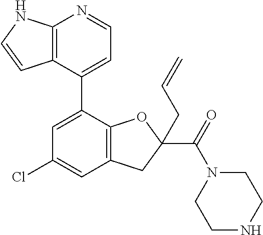 [SMILES: C=CCC1(C(=O)N2CCNCC2)Cc2cc(Cl)cc(-c3ccnc4[nH]ccc34)c2O1] | (2-allyl-5-chloro-7-(1H-pyrrolo[2,3-b]pyridin- 4-yl)-2,3-dihydrobenzofuran-2-yl)(piperazin-1- yl)methanone | C |
| 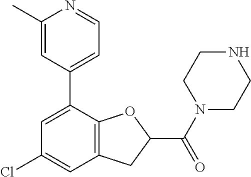 [SMILES: Cc1cc(-c2cc(Cl)cc3c2OC(C(=O)N2CCNCC2)C3)ccn1] | (5-Chloro-7-(2-methylpyridin-4-yl)-2,3- dihydrobenzofuran-2-yl)(piperazin-1- yl)methanone | C |
| 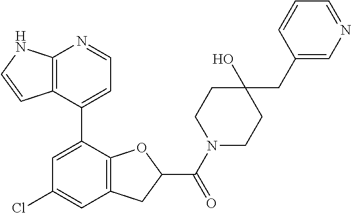 [SMILES: O=C(C1Cc2cc(Cl)cc(-c3ccnc4[nH]ccc34)c2O1)N1CCC(O)(Cc2cccnc2)CC1] | (5-chloro-7-(1H-pyrrolo[2,3-b]pyridin-4-yl)- 2,3-dihydrobenzofuran-2-yl)(4-hydroxy-4- (pyridin-3-ylmethyl)piperidin-1-yl)methanone | C |
| 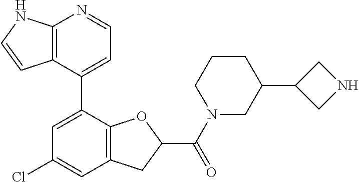 [SMILES: O=C(C1Cc2cc(Cl)cc(-c3ccnc4[nH]ccc34)c2O1)N1CCCC(C2CNC2)C1] | (3-(azetidin-3-yl)piperidin-1-yl)(5-chloro-7- (1H-pyrrolo[2,3-b]pyridin-4-yl)-2,3- dihydrobenzofuran-2-yl)methanone | C |
| 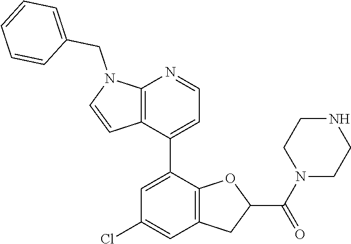 [SMILES: O=C(C1Cc2cc(Cl)cc(-c3ccnc4c3ccn4Cc3ccccc3)c2O1)N1CCNCC1] | (7-(1-Benzyl-1H-pyrrolo[2,3-b]pyridin-4-yl)-5- chloro-2,3-dihydrobenzofuran-2-yl)(piperazin- 1-yl)methanone | C |
| 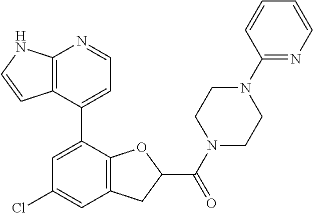 [SMILES: O=C(C1Cc2cc(Cl)cc(-c3ccnc4[nH]ccc34)c2O1)N1CCN(c2ccccn2)CC1] | (5-Chloro-7-(1H-pyrrolo[2,3-b]pyridin-4-yl)- 2,3-dihydrobenzofuran-2-yl)(4-(pyridin-2- yl)piperazin-1-yl)methanone | C |
| 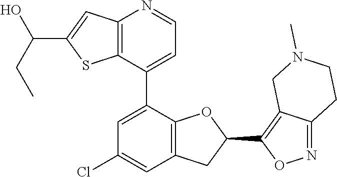 [SMILES: CCC(O)c1cc2nccc(-c3cc(Cl)cc4c3O[C@@H](c3onc5c3CN(C)CC5)C4)c2s1] | 1-(7-((R)-5-chloro-2-(5-methyl-4,5,6,7- tetrahydroisoxazolo[4,3-c]pyridin-3-yl)-2,3- dihydrobenzofuran-7-yl)thieno[3,2-b]pyridin-2- yl)propan-1-ol | A |
| 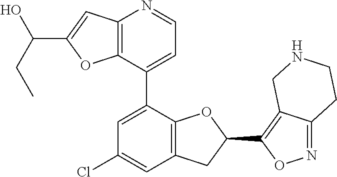 [SMILES: CCC(O)c1cc2nccc(-c3cc(Cl)cc4c3O[C@@H](c3onc5c3CNCC5)C4)c2o1] | 1-(7-((R)-5-chloro-2-(4,5,6,7- tetrahydroisoxazolo[4,3-c]pyridin-3-yl)-2,3- dihydrobenzofuran-7-yl)furo[3,2-b]pyridin-2- yl)propan-1-ol | A |
| 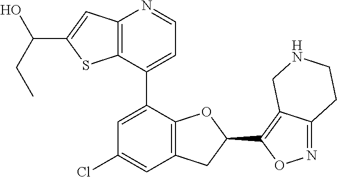 [SMILES: CCC(O)c1cc2nccc(-c3cc(Cl)cc4c3O[C@@H](c3onc5c3CNCC5)C4)c2s1] | 1-(7-((R)-5-chloro-2-(4,5,6,7- tetrahydroisoxazolo[4,3-c]pyridin-3-yl)-2,3- dihydrobenzofuran-7-yl)thieno[3,2-b]pyridin-2- yl)propan-1-ol | A |
| 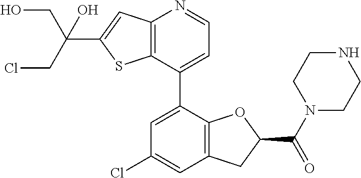 [SMILES: O=C([C@H]1Cc2cc(Cl)cc(-c3ccnc4cc(C(O)(CO)CCl)sc34)c2O1)N1CCNCC1] | ((2R)-5-chloro-7-(2-(1-chloro-2,3- dihydroxypropan-2-yl)thieno[3,2-b]pyridin-7- yl)-2,3-dihydrobenzofuran-2-yl)(piperazin-1- yl)methanone | A |
| 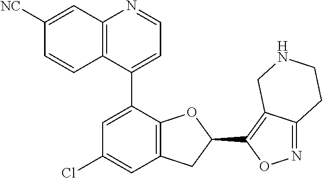 [SMILES: N#Cc1ccc2c(-c3cc(Cl)cc4c3O[C@@H](c3onc5c3CNCC5)C4)ccnc2c1] | (R)-4-(5-chloro-2-(4,5,6,7- tetrahydroisoxazolo[4,3-c]pyridin-3-yl)-2,3- dihydrobenzofuran-7-yl)quinoline-7- carbonitrile | A |
| 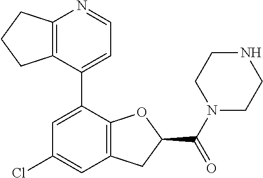 [SMILES: O=C([C@H]1Cc2cc(Cl)cc(-c3ccnc4c3CCC4)c2O1)N1CCNCC1] | (R)-(5-chloro-7-(6,7-dihydro-5H- cyclopenta[b]pyridin-4-yl)-2,3- dihydrobenzofuran-2-yl)(piperazin-1- yl)methanone | A |
| 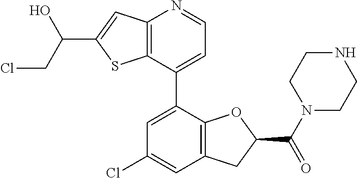 [SMILES: O=C([C@H]1Cc2cc(Cl)cc(-c3ccnc4cc(C(O)CCl)sc34)c2O1)N1CCNCC1] | ((2R)-5-chloro-7-(2-(2-chloro-1- hydroxyethyl)thieno[3,2-b]pyridin-7-yl)-2,3- dihydrobenzofuran-2-yl)(piperazin-1- yl)methanone | A |
| 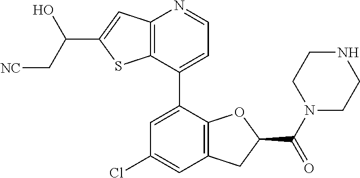 [SMILES: N#CCC(O)c1cc2nccc(-c3cc(Cl)cc4c3O[C@@H](C(=O)N3CCNCC3)C4)c2s1] | 3-(7-((R)-5-chloro-2-(piperazine-1-carbonyl)- 2,3-dihydrobenzofuran-7-yl)thieno[3,2- b]pyridin-2-yl)-3-hydroxypropanenitrile | A |
| 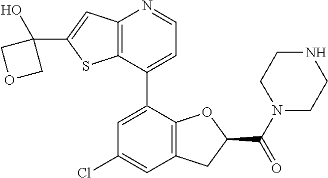 [SMILES: O=C([C@H]1Cc2cc(Cl)cc(-c3ccnc4cc(C5(O)COC5)sc34)c2O1)N1CCNCC1] | (R)-(5-chloro-7-(2-(3-hydroxyoxetan-3- yl)thieno[3,2-b]pyridin-7-yl)-2,3- dihydrobenzofuran-2-yl)(piperazin-1- yl)methanone | A |
| 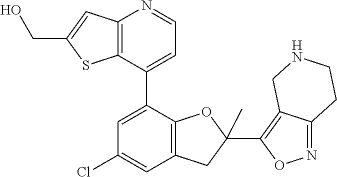 [SMILES: CC1(c2onc3c2CNCC3)Cc2cc(Cl)cc(-c3ccnc4cc(CO)sc34)c2O1] | (7-(5-chloro-2-methyl-2-(4,5,6,7- tetrahydroisoxazolo[4,3-c]pyridin-3-yl)-2,3- dihydrobenzofuran-7-yl)thieno[3,2-b]pyridin-2- yl)methanol | A |
| 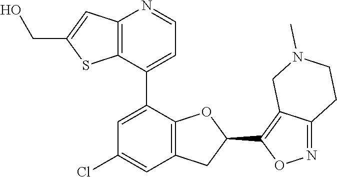 [SMILES: CN1CCc2noc([C@H]3Cc4cc(Cl)cc(-c5ccnc6cc(CO)sc56)c4O3)c2C1] | (R)-(7-(5-chloro-2-(5-methyl-4,5,6,7- tetrahydroisoxazolo[4,3-c]pyridin-3-yl)-2,3- dihydrobenzofuran-7-yl)thieno[3,2-b]pyridin-2- yl)methanol | A |
| 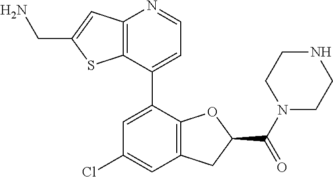 [SMILES: NCc1cc2nccc(-c3cc(Cl)cc4c3O[C@@H](C(=O)N3CCNCC3)C4)c2s1] | (R)-(7-(2-(aminomethyl)thieno[3,2-b]pyridin-7- yl)-5-chloro-2,3-dihydrobenzofuran-2- yl)(piperazin-1-yl)methanone | A |
| 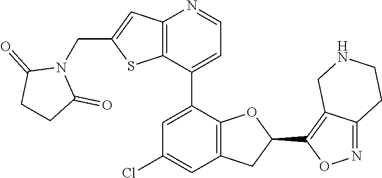 [SMILES: O=C1CCC(=O)N1Cc1cc2nccc(-c3cc(Cl)cc4c3O[C@@H](c3onc5c3CNCC5)C4)c2s1] | (R)-1-((7-(5-chloro-2-(4,5,6,7- tetrahydroisoxazolo[4,3-c]pyridin-3-yl)-2,3- dihydrobenzofuran-7-yl)thieno[3,2-b]pyridin-2- yl)methyl)pyrrolidine-2,5-dione | A |
| 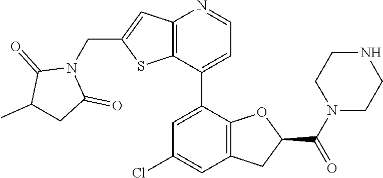 [SMILES: CC1CC(=O)N(Cc2cc3nccc(-c4cc(Cl)cc5c4O[C@@H](C(=O)N4CCNCC4)C5)c3s2)C1=O] | 1-((7-((R)-5-chloro-2-(piperazine-1-carbonyl)- 2,3-dihydrobenzofuran-7-yl)thieno[3,2- b]pyridin-2-yl)methyl)-3-methylpyrrolidine- 2,5-dione | A |
| 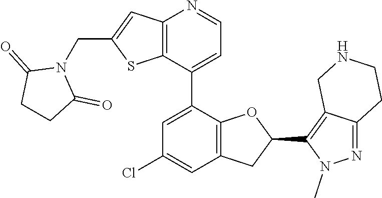 [SMILES: Cn1nc2c(c1[C@H]1Cc3cc(Cl)cc(-c4ccnc5cc(CN6C(=O)CCC6=O)sc45)c3O1)CNCC2] | (R)-1-((7-(5-chloro-2-(2-methyl-4,5,6,7- tetrahydro-2H-pyrazolo[4,3-c]pyridin-3-yl)- 2,3-dihydrobenzofuran-7-yl)thieno[3,2- b]pyridin-2-yl)methyl)pyrrolidine-2,5-dione | A |
| 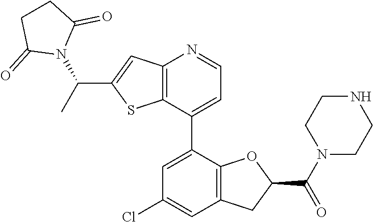 [SMILES: C[C@@H](c1cc2nccc(-c3cc(Cl)cc4c3O[C@@H](C(=O)N3CCNCC3)C4)c2s1)N1C(=O)CCC1=O] | 1-((S)-1-(7-((R)-5-chloro-2-(piperazine-1- carbonyl)-2,3-dihydrobenzofuran-7- yl)thieno[3,2-b]pyridin-2-yl)ethyl)pyrrolidine- 2,5-dione | A |
| 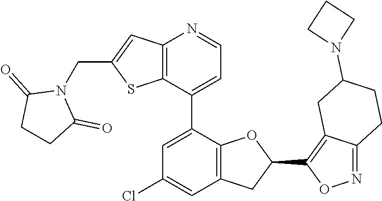 [SMILES: O=C1CCC(=O)N1Cc1cc2nccc(-c3cc(Cl)cc4c3O[C@@H](c3onc5c3CC(N3CCC3)CC5)C4)c2s1] | 1-((7-((2R)-2-(5-(azetidin-1-yl)-4,5,6,7- tetrahydrobenzo[c]isoxazol-3-yl)-5-chloro-2,3- dihydrobenzofuran-7-yl)thieno[3,2-b]pyridin-2- yl)methyl)pyrrolidine-2,5-dione | A |
| 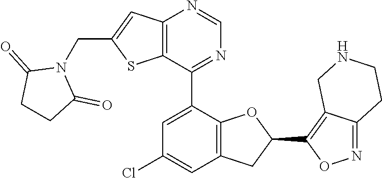 [SMILES: O=C1CCC(=O)N1Cc1cc2ncnc(-c3cc(Cl)cc4c3O[C@@H](c3onc5c3CNCC5)C4)c2s1] | (R)-1-((4-(5-chloro-2-(4,5,6,7- tetrahydroisoxazolo[4,3-c]pyridin-3-yl)-2,3- dihydrobenzofuran-7-yl)thieno[3,2- d]pyrimidin-6-yl)methyl)pyrrolidine-2,5-dione | A |
| 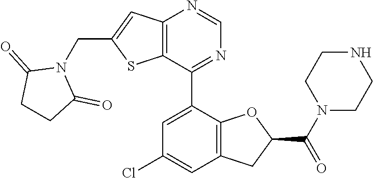 [SMILES: O=C([C@H]1Cc2cc(Cl)cc(-c3ncnc4cc(CN5C(=O)CCC5=O)sc34)c2O1)N1CCNCC1] | (R)-1-((4-(5-chloro-2-(piperazine-1-carbonyl)- 2,3-dihydrobenzofuran-7-yl)thieno[3,2- d]pyrimidin-6-yl)methyl)pyrrolidine-2,5-dione | A |
| 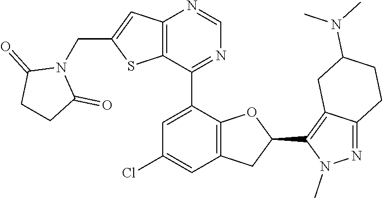 [SMILES: CN(C)C1CCc2nn(C)c([C@H]3Cc4cc(Cl)cc(-c5ncnc6cc(CN7C(=O)CCC7=O)sc56)c4O3)c2C1] | 1-((7-((2R)-5-chloro-2-(5-(dimethylamino)-2- methyl-4,5,6,7-tetrahydro-2H-indazol-3-yl)- 2,3-dihydrobenzofuran-7-yl)thieno[3,2- b]pyridin-2-yl)methyl)pyrrolidine-2,5-dione | A |
| 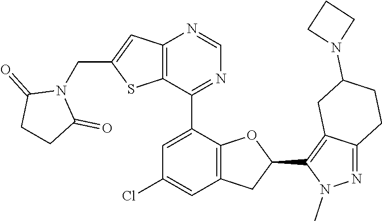 [SMILES: Cn1nc2c(c1[C@H]1Cc3cc(Cl)cc(-c4ncnc5cc(CN6C(=O)CCC6=O)sc45)c3O1)CC(N1CCC1)CC2] | 1-((7-((2R)-2-(5-(azetidin-1-yl)-2-methyl- 4,5,6,7-tetrahydro-2H-indazol-3-yl)-5-chloro- 2,3-dihydrobenzofuran-7-yl)thieno[3,2- b]pyridin-2-yl)methyl)pyrrolidine-2,5-dione | A |
| 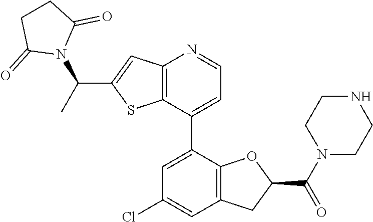 [SMILES: C[C@H](c1cc2nccc(-c3cc(Cl)cc4c3O[C@@H](C(=O)N3CCNCC3)C4)c2s1)N1C(=O)CCC1=O] | 1-((R)-1-(7-((R)-5-chloro-2-(piperazine-1- carbonyl)-2,3-dihydrobenzofuran-7- yl)thieno[3,2-b]pyridin-2-yl)ethyl)pyrrolidine- 2,5-dione | A |
| 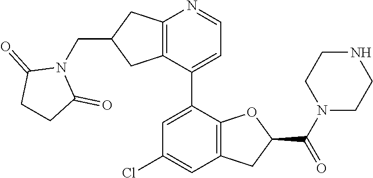 [SMILES: O=C([C@H]1Cc2cc(Cl)cc(-c3ccnc4c3CC(CN3C(=O)CCC3=O)C4)c2O1)N1CCNCC1] | 1-((4-((R)-5-chloro-2-(piperazine-1-carbonyl)- 2,3-dihydrobenzofuran-7-yl)-6,7-dihydro-5H- cyclopenta[b]pyridin-6-yl)methyl)pyrrolidine- 2,5-dione | A |
| 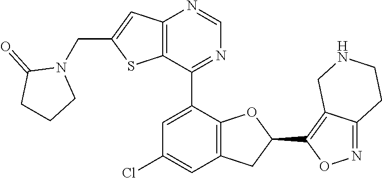 [SMILES: O=C1CCCN1Cc1cc2ncnc(-c3cc(Cl)cc4c3O[C@@H](c3onc5c3CNCC5)C4)c2s1] | (R)-1-((7-(5-chloro-2-(4,5,6,7- tetrahydroisoxazolo[4,3-c]pyridin-3-yl)-2,3- dihydrobenzofuran-7-yl)thieno[3,2-b]pyridin-2- yl)methyl)pyrrolidin-2-one | A |
| 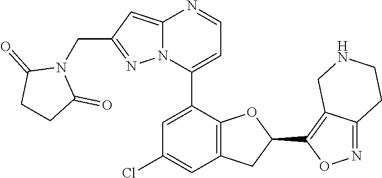 [SMILES: O=C1CCC(=O)N1Cc1cc2nccc(-c3cc(Cl)cc4c3O[C@@H](c3onc5c3CNCC5)C4)n2n1] | (R)-1-((7-(5-chloro-2-(4,5,6,7- tetrahydroisoxazolo[4,3-c]pyridin-3-yl)-2,3- dihydrobenzofuran-7-yl)pyrazolo[1,5- a]pyrimidin-2-yl)methyl)pyrrolidine-2,5-dione | A |
| 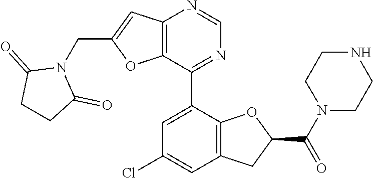 [SMILES: O=C([C@H]1Cc2cc(Cl)cc(-c3ncnc4cc(CN5C(=O)CCC5=O)oc34)c2O1)N1CCNCC1] | (R)-1-((4-(5-chloro-2-(piperazine-1-carbonyl)- 2,3-dihydrobenzofuran-7-yl)furo[3,2- d]pyrimidin-6-yl)methyl)pyrrolidine-2,5-dione | A |
| 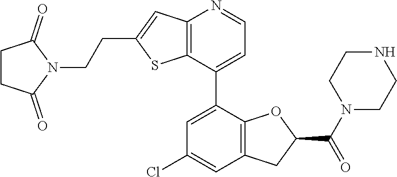 [SMILES: O=C([C@H]1Cc2cc(Cl)cc(-c3ccnc4cc(CCN5C(=O)CCC5=O)sc34)c2O1)N1CCNCC1] | (R)-1-(2-(7-(5-chloro-2-(piperazine-1- carbonyl)-2,3-dihydrobenzofuran-7- yl)thieno[3,2-b]pyridin-2-yl)ethyl)pyrrolidine- 2,5-dione | A |
| 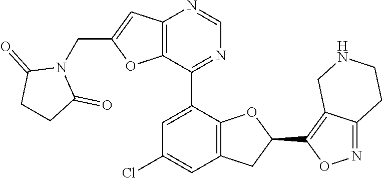 [SMILES: O=C1CCC(=O)N1Cc1cc2ncnc(-c3cc(Cl)cc4c3O[C@@H](c3onc5c3CNCC5)C4)c2o1] | (R)-1-((4-(5-chloro-2-(4,5,6,7- tetrahydroisoxazolo[4,3-c]pyridin-3-yl)-2,3- dihydrobenzofuran-7-yl)furo[3,2-d]pyrimidin- 6-yl)methyl)pyrrolidine-2,5-dione | A |
| 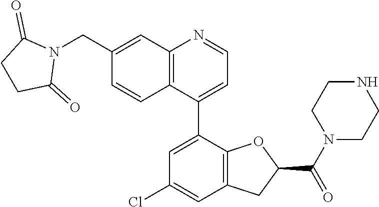 [SMILES: O=C([C@H]1Cc2cc(Cl)cc(-c3ccnc4cc(CN5C(=O)CCC5=O)ccc34)c2O1)N1CCNCC1] | (R)-1-((4-(5-chloro-2-(piperazine-1-carbonyl)- 2,3-dihydrobenzofuran-7-yl)quinolin-7- yl)methyl)pyrrolidine-2,5-dione | A |
| 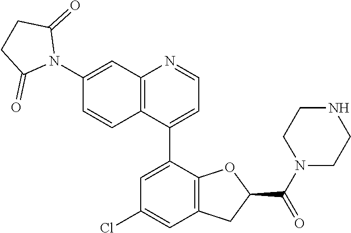 [SMILES: O=C([C@H]1Cc2cc(Cl)cc(-c3ccnc4cc(N5C(=O)CCC5=O)ccc34)c2O1)N1CCNCC1] | (R)-1-(4-(5-chloro-2-(piperazine-1-carbonyl)- 2,3-dihydrobenzofuran-7-yl)quinolin-7- yl)pyrrolidine-2,5-dione | A |
| 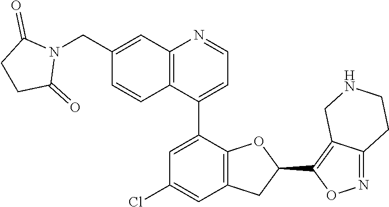 [SMILES: O=C1CCC(=O)N1Cc1ccc2c(-c3cc(Cl)cc4c3O[C@@H](c3onc5c3CNCC5)C4)ccnc2c1] | (R)-1-((4-(5-chloro-2-(4,5,6,7- tetrahydroisoxazolo[4,3-c]pyridin-3-yl)-2,3- dihydrobenzofuran-7-yl)quinolin-7- yl)methyl)pyrrolidine-2,5-dione | B |
| 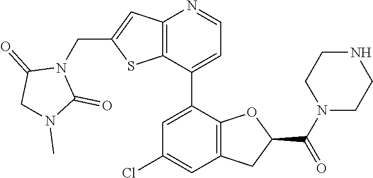 [SMILES: CN1CC(=O)N(Cc2cc3nccc(-c4cc(Cl)cc5c4O[C@@H](C(=O)N4CCNCC4)C5)c3s2)C1=O] | (R)-3-((7-(5-chloro-2-(piperazine-1-carbonyl)- 2,3-dihydrobenzofuran-7-yl)thieno[3,2- b]pyridin-2-yl)methyl)-1-methylimidazolidine- 2,4-dione | A |
| 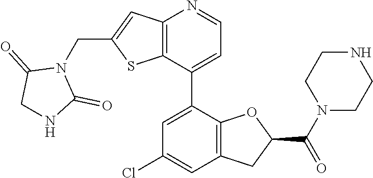 [SMILES: O=C([C@H]1Cc2cc(Cl)cc(-c3ccnc4cc(CN5C(=O)CNC5=O)sc34)c2O1)N1CCNCC1] | (R)-3-((7-(5-chloro-2-(piperazine-1-carbonyl)- 2,3-dihydrobenzofuran-7-yl)thieno[3,2- b]pyridin-2-yl)methyl)imidazolidine-2,4-dione | A |
| 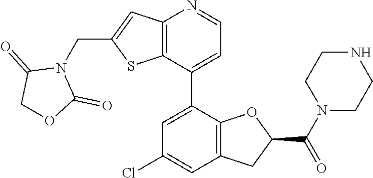 [SMILES: O=C([C@H]1Cc2cc(Cl)cc(-c3ccnc4cc(CN5C(=O)COC5=O)sc34)c2O1)N1CCNCC1] | (R)-3-((7-(5-chloro-2-(piperazine-1-carbonyl)- 2,3-dihydrobenzofuran-7-yl)thieno[3,2- b]pyridin-2-yl)methyl)oxazolidine-2,4-dione | A |
| 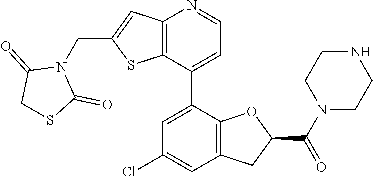 [SMILES: O=C([C@H]1Cc2cc(Cl)cc(-c3ccnc4cc(CN5C(=O)CSC5=O)sc34)c2O1)N1CCNCC1] | (R)-3-((7-(5-chloro-2-(piperazine-1-carbonyl)- 2,3-dihydrobenzofuran-7-yl)thieno[3,2- b]pyridin-2-yl)methyl)thiazolidine-2,4-dione | A |
| 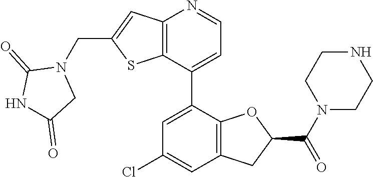 [SMILES: O=C1CN(Cc2cc3nccc(-c4cc(Cl)cc5c4O[C@@H](C(=O)N4CCNCC4)C5)c3s2)C(=O)N1] | (R)-1-((7-(5-chloro-2-(piperazine-1-carbonyl)- 2,3-dihydrobenzofuran-7-yl)thieno[3,2- b]pyridin-2-yl)methyl)imidazolidine-2,4-dione | A |
| 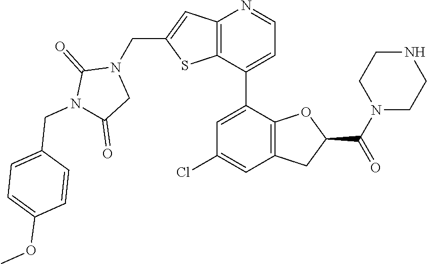 [SMILES: COc1ccc(CN2C(=O)CN(Cc3cc4nccc(-c5cc(Cl)cc6c5O[C@@H](C(=O)N5CCNCC5)C6)c4s3)C2=O)cc1] | (R)-1-((7-(5-chloro-2-(piperazine-1-carbonyl)- 2,3-dihydrobenzofuran-7-yl)thieno[3,2- b]pyridin-2-yl)methyl)-3-(4- methoxybenzyl)imidazolidine-2,4-dione | A |
| 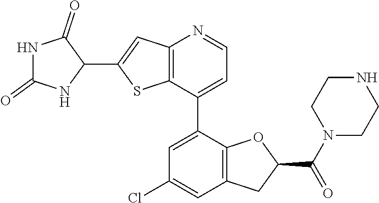 [SMILES: O=C1NC(=O)C(c2cc3nccc(-c4cc(Cl)cc5c4O[C@@H](C(=O)N4CCNCC4)C5)c3s2)N1] | 5-(7-((R)-5-chloro-2-(piperazine-1-carbonyl)- 2,3-dihydrobenzofuran-7-yl)thieno[3,2- b]pyridin-2-yl)imidazolidine-2,4-dione | A |
| 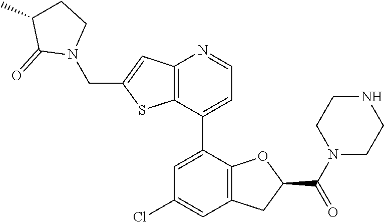 [SMILES: C[C@@H]1CCN(Cc2cc3nccc(-c4cc(Cl)cc5c4O[C@@H](C(=O)N4CCNCC4)C5)c3s2)C1=O] | (R)-1-((7-((R)-5-chloro-2-(piperazine-1- carbonyl)-2,3-dihydrobenzofuran-7- yl)thieno[3,2-b]pyridin-2-yl)methyl)-3- methylpyrrolidin-2-one | A |
| 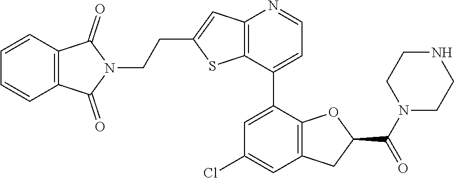 [SMILES: O=C([C@H]1Cc2cc(Cl)cc(-c3ccnc4cc(CCN5C(=O)c6ccccc6C5=O)sc34)c2O1)N1CCNCC1] | (R)-2-(2-(7-(5-chloro-2-(piperazine-1- carbonyl)-2,3-dihydrobenzofuran-7- yl)thieno[3,2-b]pyridin-2-yl)ethyl)isoindoline- 1,3-dione | A |
| 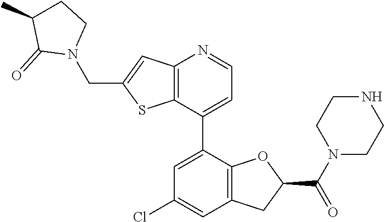 [SMILES: C[C@H]1CCN(Cc2cc3nccc(-c4cc(Cl)cc5c4O[C@@H](C(=O)N4CCNCC4)C5)c3s2)C1=O] | (S)-1-((7-((R)-5-chloro-2-(piperazine-1- carbonyl)-2,3-dihydrobenzofuran-7- yl)thieno[3,2-b]pyridin-2-yl)methyl)-3- methylpyrrolidin-2-one | A |
| 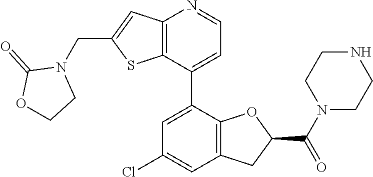 [SMILES: O=C1OCCN1Cc1cc2nccc(-c3cc(Cl)cc4c3O[C@@H](C(=O)N3CCNCC3)C4)c2s1] | (R)-3-((7-(5-chloro-2-(piperazine-1-carbonyl)- 2,3-dihydrobenzofuran-7-yl)thieno[3,2- b]pyridin-2-yl)methyl)oxazolidin-2-one | A |
| 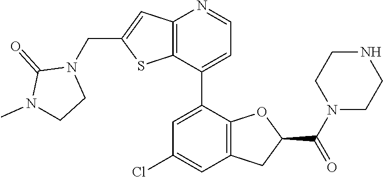 [SMILES: CN1CCN(Cc2cc3nccc(-c4cc(Cl)cc5c4O[C@@H](C(=O)N4CCNCC4)C5)c3s2)C1=O] | (R)-1-((7-(5-chloro-2-(piperazine-1-carbonyl)- 2,3-dihydrobenzofuran-7-yl)thieno[3,2- b]pyridin-2-yl)methyl)-3-methylimidazolidin- 2-one | A |
| 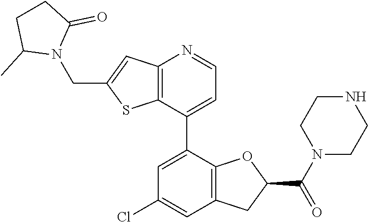 [SMILES: CC1CCC(=O)N1Cc1cc2nccc(-c3cc(Cl)cc4c3O[C@@H](C(=O)N3CCNCC3)C4)c2s1] | 1-((7-((R)-5-chloro-2-(piperazine-1-carbonyl)- 2,3-dihydrobenzofuran-7-yl)thieno[3,2- b]pyridin-2-yl)methyl)-5-methylpyrrolidin-2- one | A |
| 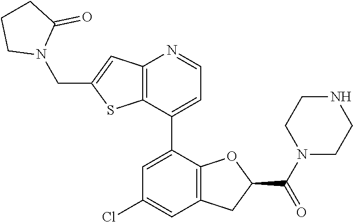 [SMILES: O=C1CCCN1Cc1cc2nccc(-c3cc(Cl)cc4c3O[C@@H](C(=O)N3CCNCC3)C4)c2s1] | (R)-1-((7-(5-chloro-2-(piperazine-1-carbonyl)- 2,3-dihydrobenzofuran-7-yl)thieno[3,2- b]pyridin-2-yl)methyl)pyrrolidin-2-one | A |
| 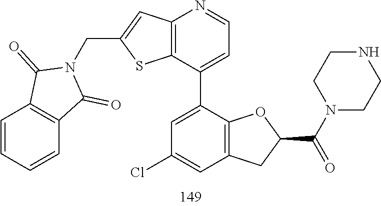 [SMILES: O=C([C@H]1Cc2cc(Cl)cc(-c3ccnc4cc(CN5C(=O)c6ccccc6C5=O)sc34)c2O1)N1CCNCC1] | (R)-2-((7-(5-chloro-2-(piperazine-1-carbonyl)- 2,3-dihydrobenzofuran-7-yl)thieno[3,2- b]pyridin-2-yl)methyl)isoindoline-1,3-dione | A |
| 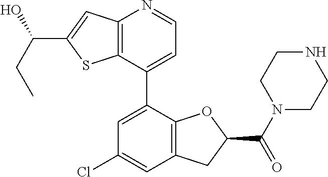 [SMILES: CC[C@H](O)c1cc2nccc(-c3cc(Cl)cc4c3O[C@@H](C(=O)N3CCNCC3)C4)c2s1] | ((R)-5-chloro-7-(2-((S)-1- hydroxypropyl)thieno[3,2-b]pyridin-7-yl)-2,3- dihydrobenzofuran-2-yl)(piperazin-1- yl)methanone | A |
| 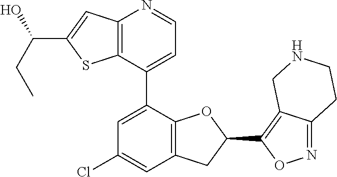 [SMILES: CC[C@H](O)c1cc2nccc(-c3cc(Cl)cc4c3O[C@@H](c3onc5c3CNCC5)C4)c2s1] | (S)-1-(7-((R)-5-chloro-2-(4,5,6,7- tetrahydroisoxazolo[4,3-c]pyridin-3-yl)-2,3- dihydrobenzofuran-7-yl)thieno[3,2-b]pyridin-2- yl)propan-1-ol | A |
| 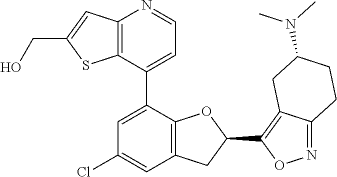 [SMILES: CN(C)[C@@H]1CCc2noc([C@H]3Cc4cc(Cl)cc(-c5ccnc6cc(CO)sc56)c4O3)c2C1] | (7-((R)-5-chloro-2-((R)-5-(dimethylamino)- 4,5,6,7-tetrahydrobenzo[c]isoxazol-3-yl)-2,3- dihydrobenzofuran-7-yl)thieno[3,2-b]pyridin-2- yl)methanol | A |
| 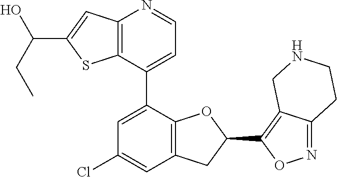 [SMILES: CCC(O)c1cc2nccc(-c3cc(Cl)cc4c3O[C@@H](c3onc5c3CNCC5)C4)c2s1] | 1-(7-((R)-5-chloro-2-(4,5,6,7- tetrahydroisoxazolo[4,3-c]pyridin-3-yl)-2,3- dihydrobenzofuran-7-yl)thieno[3,2-b]pyridin-2- yl)propan-1-ol | A |
| 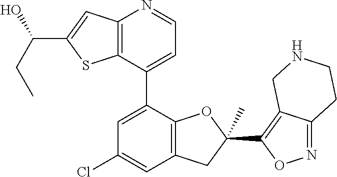 [SMILES: CC[C@H](O)c1cc2nccc(-c3cc(Cl)cc4c3O[C@@](C)(c3onc5c3CNCC5)C4)c2s1] | (S)-1-(7-((R)-5-chloro-2-methyl-2-(4,5,6,7- tetrahydroisoxazolo[4,3-c]pyridin-3-yl)-2,3- dihydrobenzofuran-7-yl)thieno[3,2-b]pyridin-2- yl)propan-1-ol | A |
| 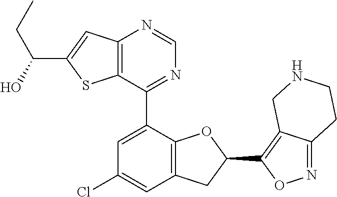 [SMILES: CC[C@@H](O)c1cc2ncnc(-c3cc(Cl)cc4c3O[C@@H](c3onc5c3CNCC5)C4)c2s1] | (R)-1-(4-((R)-5-chloro-2-(4,5,6,7- tetrahydroisoxazolo[4,3-c]pyridin-3-yl)-2,3- dihydrobenzofuran-7-yl)thieno[3,2- d]pyrimidin-6-yl)propan-1-ol | A |
| 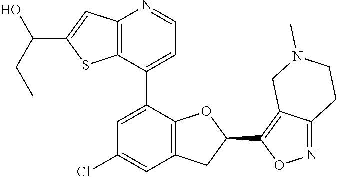 [SMILES: CCC(O)c1cc2nccc(-c3cc(Cl)cc4c3O[C@@H](c3onc5c3CN(C)CC5)C4)c2s1] | 1-(7-((R)-5-chloro-2-(5-methyl-4,5,6,7- tetrahydroisoxazolo[4,3-c]pyridin-3-yl)-2,3- dihydrobenzofuran-7-yl)thieno[3,2-b]pyridin-2- yl)propan-1-ol | A |
| 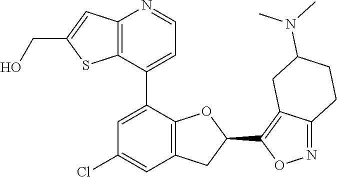 [SMILES: CN(C)C1CCc2noc([C@H]3Cc4cc(Cl)cc(-c5ccnc6cc(CO)sc56)c4O3)c2C1] | (7-((2R)-5-chloro-2-(5-(dimethylamino)- 4,5,6,7-tetrahydrobenzo[c]isoxazol-3-yl)-2,3- dihydrobenzofuran-7-yl)thieno[3,2-b]pyridin-2- yl)methanol | A |
| 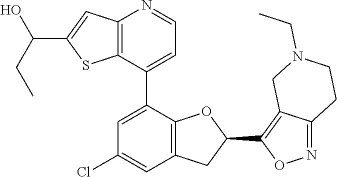 [SMILES: CCC(O)c1cc2nccc(-c3cc(Cl)cc4c3O[C@@H](c3onc5c3CN(CC)CC5)C4)c2s1] | 1-(7-((R)-5-chloro-2-(5-ethyl-4,5,6,7- tetrahydroisoxazolo[4,3-c]pyridin-3-yl)-2,3- dihydrobenzofuran-7-yl)thieno[3,2-b]pyridin-2- yl)propan-1-ol | A |
| 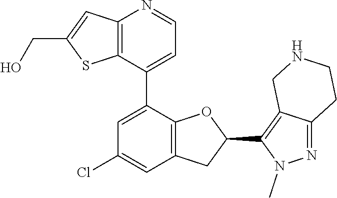 [SMILES: Cn1nc2c(c1[C@H]1Cc3cc(Cl)cc(-c4ccnc5cc(CO)sc45)c3O1)CNCC2] | (R)-(7-(5-chloro-2-(2-methyl-4,5,6,7- tetrahydro-2H-pyrazolo[4,3-c]pyridin-3-yl)- 2,3-dihydrobenzofuran-7-yl)thieno[3,2- b]pyridin-2-yl)methanol | A |
| 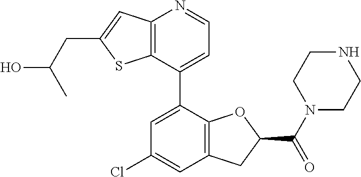 [SMILES: CC(O)Cc1cc2nccc(-c3cc(Cl)cc4c3O[C@@H](C(=O)N3CCNCC3)C4)c2s1] | ((2R)-5-chloro-7-(2-(2- hydroxypropyl)thieno[3,2-b]pyridin-7-yl)-2,3- dihydrobenzofuran-2-yl)(piperazin-1- yl)methanone | A |
| 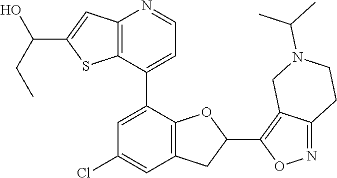 [SMILES: CCC(O)c1cc2nccc(-c3cc(Cl)cc4c3OC(c3onc5c3CN(C(C)C)CC5)C4)c2s1] | 1-(7-(5-chloro-2-(5-isopropyl-4,5,6,7- tetrahydroisoxazolo[4,3-c]pyridin-3-yl)-2,3- dihydrobenzofuran-7-yl)thieno[3,2-b]pyridin-2- yl)propan-1-ol | A |
| 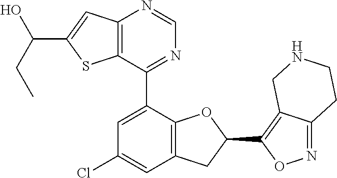 [SMILES: CCC(O)c1cc2ncnc(-c3cc(Cl)cc4c3O[C@@H](c3onc5c3CNCC5)C4)c2s1] | 1-(4-((R)-5-chloro-2-(4,5,6,7- tetrahydroisoxazolo[4,3-c]pyridin-3-yl)-2,3- dihydrobenzofuran-7-yl)thieno[3,2- d]pyrimidin-6-yl)propan-1-ol | A |
| 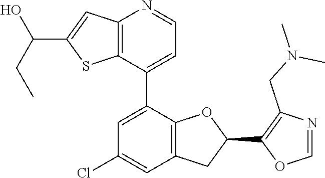 [SMILES: CCC(O)c1cc2nccc(-c3cc(Cl)cc4c3O[C@@H](c3ocnc3CN(C)C)C4)c2s1] | 1-(7-((R)-5-chloro-2-(4- ((dimethylamino)methyl)oxazol-5-yl)-2,3- dihydrobenzofuran-7-yl)thieno[3,2-b]pyridin-2- yl)propan-1-ol | A |
| 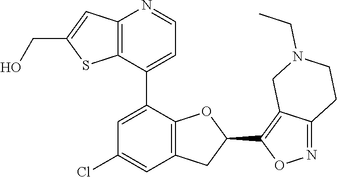 [SMILES: CCN1CCc2noc([C@H]3Cc4cc(Cl)cc(-c5ccnc6cc(CO)sc56)c4O3)c2C1] | (R)-(7-(5-chloro-2-(5-ethyl-4,5,6,7- tetrahydroisoxazolo[4,3-c]pyridin-3-yl)-2,3- dihydrobenzofuran-7-yl)thieno[3,2-b]pyridin-2- yl)methanol | A |
| 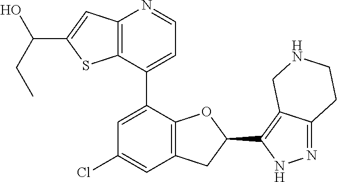 [SMILES: CCC(O)c1cc2nccc(-c3cc(Cl)cc4c3O[C@@H](c3[nH]nc5c3CNCC5)C4)c2s1] | 1-(7-((R)-5-chloro-2-(4,5,6,7-tetrahydro-2H- pyrazolo[4,3-c]pyridin-3-yl)-2,3- dihydrobenzofuran-7-yl)thieno[3,2-b]pyridin-2- yl)propan-1-ol | A |
| 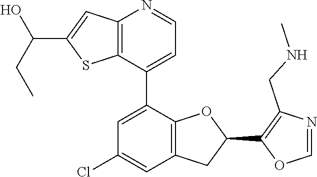 [SMILES: CCC(O)c1cc2nccc(-c3cc(Cl)cc4c3O[C@@H](c3ocnc3CNC)C4)c2s1] | 1-(7-((R)-5-chloro-2-(4- ((methylamino)methyl)oxazol-5-yl)-2,3- dihydrobenzofuran-7-yl)thieno[3,2-b]pyridin-2- yl)propan-1-ol | A |
| 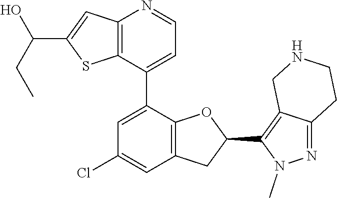 [SMILES: CCC(O)c1cc2nccc(-c3cc(Cl)cc4c3O[C@@H](c3c5c(nn3C)CCNC5)C4)c2s1] | 1-(7-((R)-5-chloro-2-(2-methyl-4,5,6,7- tetrahydro-2H-pyrazolo[4,3-c]pyridin-3-yl)- 2,3-dihydrobenzofuran-7-yl)thieno[3,2- b]pyridin-2-yl)propan-1-ol | A |
| 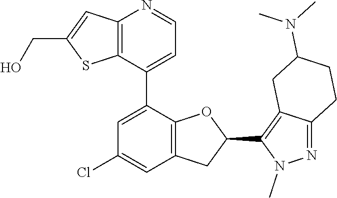 [SMILES: CN(C)C1CCc2nn(C)c([C@H]3Cc4cc(Cl)cc(-c5ccnc6cc(CO)sc56)c4O3)c2C1] | (7-((2R)-5-chloro-2-(5-(dimethylamino)-2- methyl-4,5,6,7-tetrahydro-2H-indazol-3-yl)- 2,3-dihydrobenzofuran-7-yl)thieno[3,2- b]pyridin-2-yl)methanol | A |
| 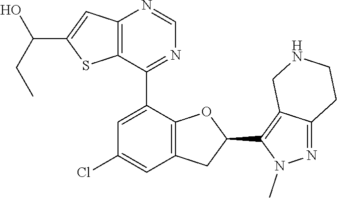 [SMILES: CCC(O)c1cc2ncnc(-c3cc(Cl)cc4c3O[C@@H](c3c5c(nn3C)CCNC5)C4)c2s1] | 1-(4-((R)-5-chloro-2-(2-methyl-4,5,6,7- tetrahydro-2H-pyrazolo[4,3-c]pyridin-3-yl)- 2,3-dihydrobenzofuran-7-yl)thieno[3,2- d]pyrimidin-6-yl)propan-1-ol | A |
| 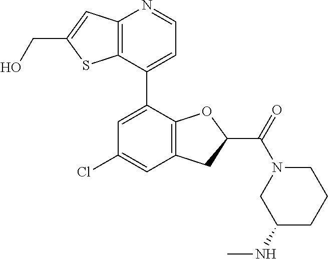 [SMILES: CN[C@H]1CCCN(C(=O)[C@H]2Cc3cc(Cl)cc(-c4ccnc5cc(CO)sc45)c3O2)C1] | ((R)-5-chloro-7-(2-(hydroxymethyl)thieno[3,2- b]pyridin-7-yl)-2,3-dihydrobenzofuran-2- yl)((S)-3-(methylamino)piperidin-1- yl)methanone | A |
| 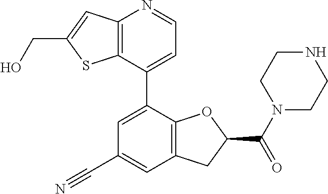 [SMILES: N#Cc1cc2c(c(-c3ccnc4cc(CO)sc34)c1)O[C@@H](C(=O)N1CCNCC1)C2] | (R)-7-(2-(hydroxymethyl)thieno[3,2-b]pyridin- 7-yl)-2-(piperazine-1-carbonyl)-2,3- dihydrobenzofuran-5-carbonitrile | A |
| 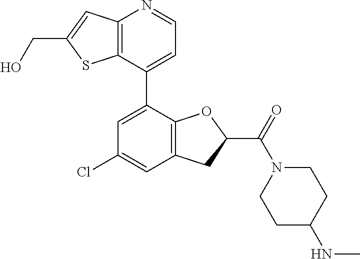 [SMILES: CNC1CCN(C(=O)[C@H]2Cc3cc(Cl)cc(-c4ccnc5cc(CO)sc45)c3O2)CC1] | (R)-(5-chloro-7-(2-(hydroxymethyl)thieno[3,2- b]pyridin-7-yl)-2,3-dihydrobenzofuran-2-yl)(4- (methylamino)piperidin-1-yl)methanone | A |
| 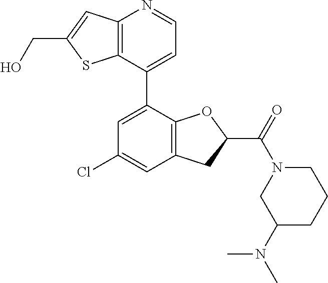 [SMILES: CN(C)C1CCCN(C(=O)[C@H]2Cc3cc(Cl)cc(-c4ccnc5cc(CO)sc45)c3O2)C1] | ((R)-5-chloro-7-(2-(hydroxymethyl)thieno[3,2- b]pyridin-7-yl)-2,3-dihydrobenzofuran-2-yl)(3- (dimethylamino)piperidin-1-yl)methanone | A |
| 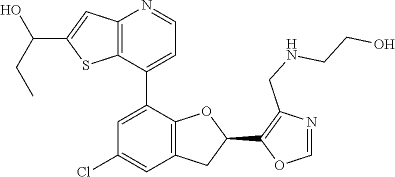 [SMILES: CCC(O)c1cc2nccc(-c3cc(Cl)cc4c3O[C@@H](c3ocnc3CNCCO)C4)c2s1] | 1-(7-((R)-5-chloro-2-(4-(((2- hydroxyethyl)amino)methyl)oxazol-5-yl)-2,3- dihydrobenzofuran-7-yl)thieno[3,2-b]pyridin-2- yl)propan-1-ol | A |
| 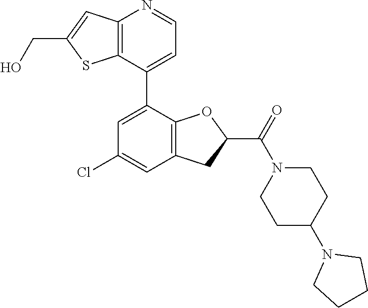 [SMILES: O=C([C@H]1Cc2cc(Cl)cc(-c3ccnc4cc(CO)sc34)c2O1)N1CCC(N2CCCC2)CC1] | (R)-(5-chloro-7-(2-(hydroxymethyl)thieno[3,2- b]pyridin-7-yl)-2,3-dihydrobenzofuran-2-yl)(4- (pyrrolidin-1-yl)piperidin-1-yl)methanone | A |
| 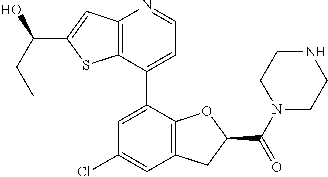 [SMILES: CC[C@@H](O)c1cc2nccc(-c3cc(Cl)cc4c3O[C@@H](C(=O)N3CCNCC3)C4)c2s1] | ((R)-5-chloro-7-(2-((R)-1- hydroxypropyl)thieno[3,2-b]pyridin-7-yl)-2,3- dihydrobenzofuran-2-yl)(piperazin-1- yl)methanone | A |
| 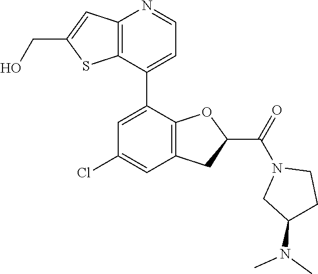 [SMILES: CN(C)[C@@H]1CCN(C(=O)[C@H]2Cc3cc(Cl)cc(-c4ccnc5cc(CO)sc45)c3O2)C1] | ((R)-5-chloro-7-(2-(hydroxymethyl)thieno[3,2- b]pyridin-7-yl)-2,3-dihydrobenzofuran-2- yl)((R)-3-(dimethylamino)pyrrolidin-1- yl)methanone | A |
| 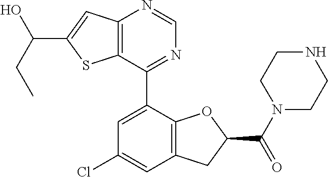 [SMILES: CCC(O)c1cc2ncnc(-c3cc(Cl)cc4c3O[C@@H](C(=O)N3CCNCC3)C4)c2s1] | ((2R)-5-chloro-7-(6-(1- hydroxypropyl)thieno[3,2-d]pyrimidin-4-yl)- 2,3-dihydrobenzofuran-2-yl)(piperazin-1- yl)methanone | A |
| 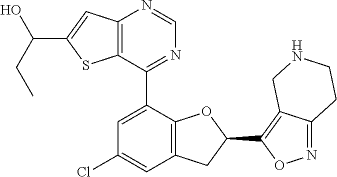 [SMILES: CCC(O)c1cc2ncnc(-c3cc(Cl)cc4c3O[C@@H](c3onc5c3CNCC5)C4)c2s1] | 1-(4-((R)-5-chloro-2-(4,5,6,7- tetrahydroisoxazolo[4,3-c]pyridin-3-yl)-2,3- dihydrobenzofuran-7-yl)thieno[3,2- d]pyrimidin-6-yl)propan-1-ol | A |
| 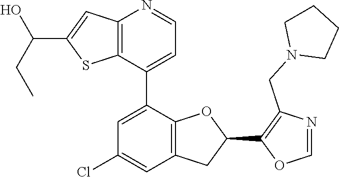 [SMILES: CCC(O)c1cc2nccc(-c3cc(Cl)cc4c3O[C@@H](c3ocnc3CN3CCCC3)C4)c2s1] | 1-(7-((R)-5-chloro-2-(4-(pyrrolidin-1- ylmethyl)oxazol-5-yl)-2,3-dihydrobenzofuran- 7-yl)thieno[3,2-b]pyridin-2-yl)propan-1-ol | A |
| 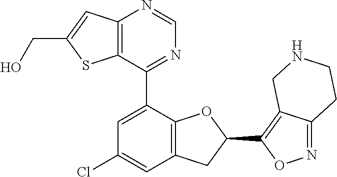 [SMILES: OCc1cc2ncnc(-c3cc(Cl)cc4c3O[C@@H](c3onc5c3CNCC5)C4)c2s1] | (R)-(4-(5-chloro-2-(4,5,6,7- tetrahydroisoxazolo[4,3-c]pyridin-3-yl)-2,3- dihydrobenzofuran-7-yl)thieno[3,2- d]pyrimidin-6-yl)methanol | A |
| 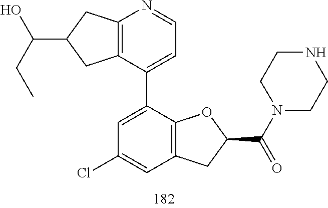 [SMILES: CCC(O)C1Cc2nccc(-c3cc(Cl)cc4c3O[C@@H](C(=O)N3CCNCC3)C4)c2C1] | ((2R)-5-chloro-7-(6-(1-hydroxypropyl)-6,7- dihydro-5H-cyclopenta[b]pyridin-4-yl)-2,3- dihydrobenzofuran-2-yl)(piperazin-1- yl)methanone | A |
| 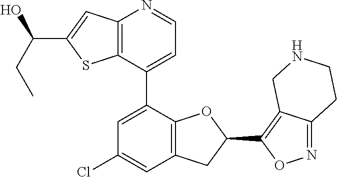 [SMILES: CC[C@@H](O)c1cc2nccc(-c3cc(Cl)cc4c3O[C@@H](c3onc5c3CNCC5)C4)c2s1] | (R)-1-(7-((R)-5-chloro-2-(4,5,6,7- tetrahydroisoxazolo[4,3-c]pyridin-3-yl)-2,3- dihydrobenzofuran-7-yl)thieno[3,2-b]pyridin-2- yl)propan-1-ol | A |
| 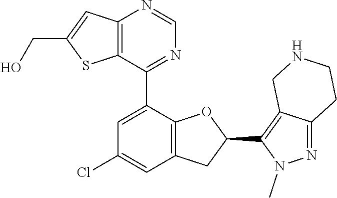 [SMILES: Cn1nc2c(c1[C@H]1Cc3cc(Cl)cc(-c4ncnc5cc(CO)sc45)c3O1)CNCC2] | (R)-(4-(5-chloro-2-(2-methyl-4,5,6,7- tetrahydro-2H-pyrazolo[4,3-c]pyridin-3-yl)- 2,3-dihydrobenzofuran-7-yl)thieno[3,2- d]pyrimidin-6-yl)methanol | A |
| 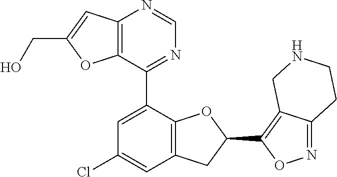 [SMILES: OCc1cc2ncnc(-c3cc(Cl)cc4c3O[C@@H](c3onc5c3CNCC5)C4)c2o1] | (R)-(4-(5-chloro-2-(4,5,6,7- tetrahydroisoxazolo[4,3-c]pyridin-3-yl)-2,3- dihydrobenzofuran-7-yl)furo[3,2-d]pyrimidin- 6-yl)methanol | A |
| 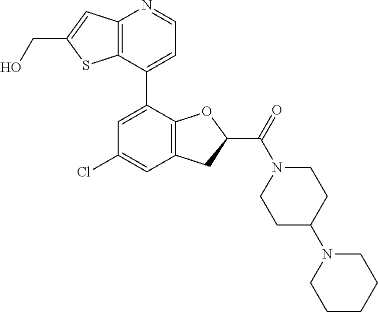 [SMILES: O=C([C@H]1Cc2cc(Cl)cc(-c3ccnc4cc(CO)sc34)c2O1)N1CCC(N2CCCCC2)CC1] | (R)-[1,4′-bipiperidin]-1′-yl(5-chloro-7-(2- (hydroxymethyl)thieno[3,2-b]pyridin-7-yl)-2,3- dihydrobenzofuran-2-yl)methanone | A |
| 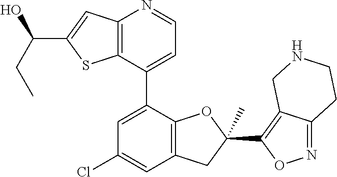 [SMILES: CC[C@@H](O)c1cc2nccc(-c3cc(Cl)cc4c3O[C@@](C)(c3onc5c3CNCC5)C4)c2s1] | (R)-1-(7-((R)-5-chloro-2-methyl-2-(4,5,6,7- tetrahydroisoxazolo[4,3-c]pyridin-3-yl)-2,3- dihydrobenzofuran-7-yl)thieno[3,2-b]pyridin-2- yl)propan-1-ol | A |
| 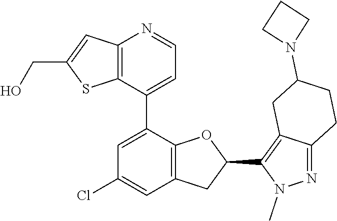 [SMILES: Cn1nc2c(c1[C@H]1Cc3cc(Cl)cc(-c4ccnc5cc(CO)sc45)c3O1)CC(N1CCC1)CC2] | (7-((2R)-2-(5-(azetidin-1-yl)-2-methyl-4,5,6,7- tetrahydro-2H-indazol-3-yl)-5-chloro-2,3- dihydrobenzofuran-7-yl)thieno[3,2-b]pyridin-2- yl)methanol | A |
| 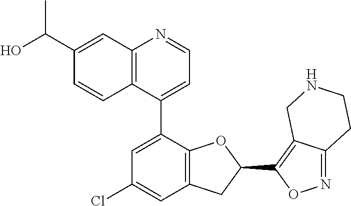 [SMILES: CC(O)c1ccc2c(-c3cc(Cl)cc4c3O[C@@H](c3onc5c3CNCC5)C4)ccnc2c1] | 1-(4-((R)-5-chloro-2-(4,5,6,7- tetrahydroisoxazolo[4,3-c]pyridin-3-yl)-2,3- dihydrobenzofuran-7-yl)quinolin-7-yl)ethan-1- ol | A |
| 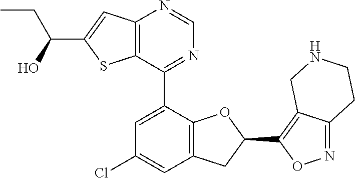 [SMILES: CC[C@H](O)c1cc2ncnc(-c3cc(Cl)cc4c3O[C@@H](c3onc5c3CNCC5)C4)c2s1] | (S)-1-(4-((R)-5-chloro-2-(4,5,6,7- tetrahydroisoxazolo[4,3-c]pyridin-3-yl)-2,3- dihydrobenzofuran-7-yl)thieno[3,2- d]pyrimidin-6-yl)propan-1-ol | A |
| 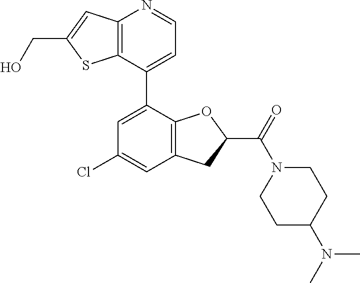 [SMILES: CN(C)C1CCN(C(=O)[C@H]2Cc3cc(Cl)cc(-c4ccnc5cc(CO)sc45)c3O2)CC1] | (R)-(5-chloro-7-(2-(hydroxymethyl)thieno[3,2- b]pyridin-7-yl)-2,3-dihydrobenzofuran-2-yl)(4- (dimethylamino)piperidin-1-yl)methanone | A |
| 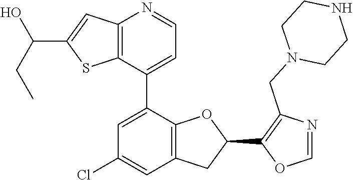 [SMILES: CCC(O)c1cc2nccc(-c3cc(Cl)cc4c3O[C@@H](c3ocnc3CN3CCNCC3)C4)c2s1] | 1-(7-((R)-5-chloro-2-(4-(piperazin-1- ylmethyl)oxazol-5-yl)-2,3-dihydrobenzofuran- 7-yl)thieno[3,2-b]pyridin-2-yl)propan-1-ol | A |
| 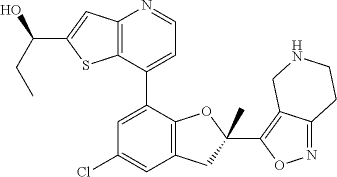 [SMILES: CC[C@@H](O)c1cc2nccc(-c3cc(Cl)cc4c3O[C@@](C)(c3onc5c3CNCC5)C4)c2s1] | (R)-1-(7-((S)-5-chloro-2-methyl-2-(4,5,6,7- tetrahydroisoxazolo[4,3-c]pyridin-3-yl)-2,3- dihydrobenzofuran-7-yl)thieno[3,2-b]pyridin-2- yl)propan-1-ol | A |
| 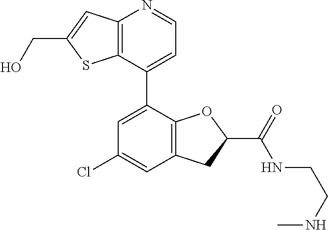 [SMILES: CNCCNC(=O)[C@H]1Cc2cc(Cl)cc(-c3ccnc4cc(CO)sc34)c2O1] | (R)-5-chloro-7-(2-(hydroxymethyl)thieno[3,2- b]pyridin-7-yl)-N-(2-(methylamino)ethyl)-2,3- dihydrobenzofuran-2-carboxamide | A |
| 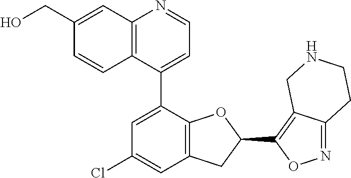 [SMILES: OCc1ccc2c(-c3cc(Cl)cc4c3O[C@@H](c3onc5c3CNCC5)C4)ccnc2c1] | (R)-(4-(5-chloro-2-(4,5,6,7- tetrahydroisoxazolo[4,3-c]pyridin-3-yl)-2,3- dihydrobenzofuran-7-yl)quinolin-7-yl)methanol | A |
| 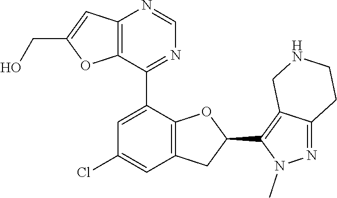 [SMILES: Cn1nc2c(c1[C@H]1Cc3cc(Cl)cc(-c4ncnc5cc(CO)oc45)c3O1)CNCC2] | (R)-(4-(5-chloro-2-(2-methyl-4,5,6,7- tetrahydro-2H-pyrazolo [4,3-c]pyridin-3-yl)- 2,3-dihydrobenzofuran-7-yl)furo[3,2- d]pyrimidin-6-yl)methanol | A |
| 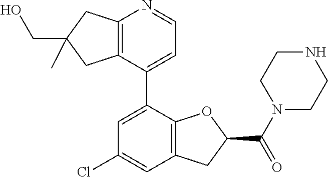 [SMILES: CC1(CO)Cc2nccc(-c3cc(Cl)cc4c3O[C@@H](C(=O)N3CCNCC3)C4)c2C1] | ((2R)-5-chloro-7-(6-(hydroxymethyl)-6- methyl-6,7-dihydro-5H-cyclopenta[b]pyridin-4- yl)-2,3-dihydrobenzofuran-2-yl)(piperazin-1- yl)methanone | A |
| 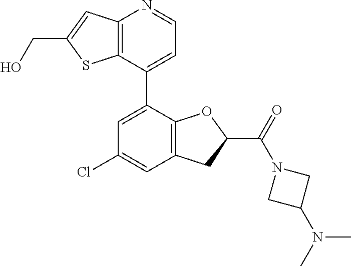 [SMILES: CN(C)C1CN(C(=O)[C@H]2Cc3cc(Cl)cc(-c4ccnc5cc(CO)sc45)c3O2)C1] | (R)-(5-chloro-7-(2-(hydroxymethyl)thieno[3,2- b]pyridin-7-yl)-2,3-dihydrobenzofuran-2-yl)(3- (dimethylamino)azetidin-1-yl)methanone | A |
| 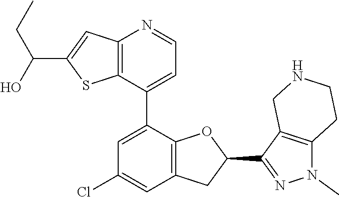 [SMILES: CCC(O)c1cc2nccc(-c3cc(Cl)cc4c3O[C@@H](c3nn(C)c5c3CNCC5)C4)c2s1] | 1-(7-((R)-5-chloro-2-(1-methyl-4,5,6,7- tetrahydro-1H-pyrazolo[4,3-c]pyridin-3-yl)- 2,3-dihydrobenzofuran-7-yl)thieno[3,2- b]pyridin-2-yl)propan-1-ol | A |
| 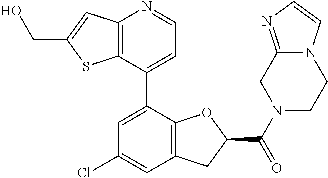 [SMILES: O=C([C@H]1Cc2cc(Cl)cc(-c3ccnc4cc(CO)sc34)c2O1)N1CCn2ccnc2C1] | (R)-(5-chloro-7-(2-(hydroxymethyl)thieno[3,2- b]pyridin-7-yl)-2,3-dihydrobenzofuran-2- yl)(5,6-dihydroimidazo[1,2-a]pyrazin-7(8H)- yl)methanone | A |
| 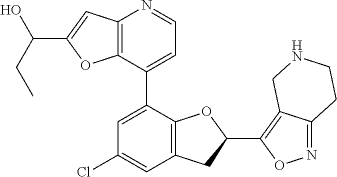 [SMILES: CCC(O)c1cc2nccc(-c3cc(Cl)cc4c3O[C@@H](c3onc5c3CNCC5)C4)c2o1] | 1-(7-((R)-5-chloro-2-(4,5,6,7- tetrahydroisoxazolo[4,3-c]pyridin-3-yl)-2,3- dihydrobenzofuran-7-yl)furo[3,2-b]pyridin-2- yl)propan-1-ol | A |
| 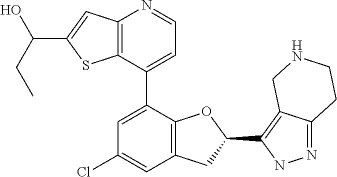 | 1-(7-((S)-5-chloro-2-(4,5,6,7- tetrahydroisoxazolo[4,3-c]pyridin-3-yl)-2,3- dihydrobenzofuran-7-yl)thieno[3,2-b]pyridin-2- yl)propan-1-ol | B |
| 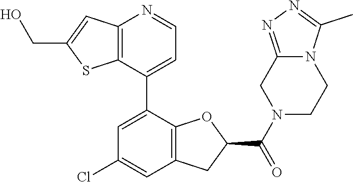 [SMILES: Cc1nnc2n1CCN(C(=O)[C@H]1Cc3cc(Cl)cc(-c4ccnc5cc(CO)sc45)c3O1)C2] | (R)-(5-chloro-7-(2-(hydroxymethyl)thieno[3,2- b]pyridin-7-yl)-2,3-dihydrobenzofuran-2-yl)(3- methyl-5,6-dihydro-[1,2,4]triazolo[4,3- a]pyrazin-7(8H)-yl)methanone | B |
| 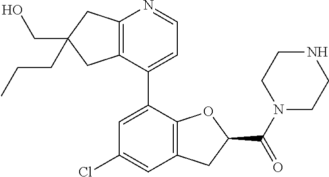 [SMILES: CCCC1(CO)Cc2nccc(-c3cc(Cl)cc4c3O[C@@H](C(=O)N3CCNCC3)C4)c2C1] | ((2R)-5-chloro-7-(6-(hydroxymethyl)-6-propyl- 6,7-dihydro-5H-cyclopenta[b]pyridin-4-yl)-2,3- dihydrobenzofuran-2-yl)(piperazin-1- yl)methanone | B |
| 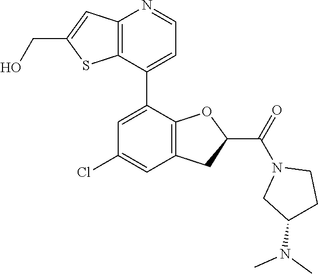 [SMILES: CN(C)[C@H]1CCN(C(=O)[C@H]2Cc3cc(Cl)cc(-c4ccnc5cc(CO)sc45)c3O2)C1] | ((R)-5-chloro-7-(2-(hydroxymethyl)thieno[3,2- b]pyridin-7-yl)-2,3-dihydrobenzofuran-2- yl)((S)-3-(dimethylamino)pyrrolidin-1- yl)methanone | B |
| 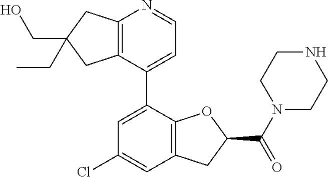 [SMILES: CCC1(CO)Cc2nccc(-c3cc(Cl)cc4c3O[C@@H](C(=O)N3CCNCC3)C4)c2C1] | ((2R)-5-chloro-7-(6-ethyl-6-(hydroxymethyl)- 6,7-dihydro-5H-cyclopenta[b]pyridin-4-yl)-2,3- dihydrobenzofuran-2-yl)(piperazin-1- yl)methanone | B |
| 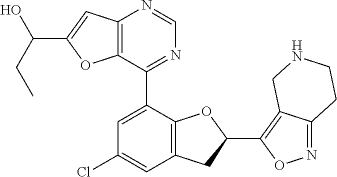 [SMILES: CCC(O)c1cc2ncnc(-c3cc(Cl)cc4c3O[C@@H](c3onc5c3CNCC5)C4)c2o1] | 1-(4-((R)-5-chloro-2-(4,5,6,7- tetrahydroisoxazolo[4,3-c]pyridin-3-yl)-2,3- dihydrobenzofuran-7-yl)furo[3,2-d]pyrimidin- 6-yl)propan-1-ol | B |
| 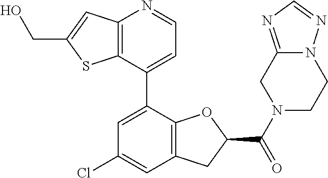 [SMILES: O=C([C@H]1Cc2cc(Cl)cc(-c3ccnc4cc(CO)sc34)c2O1)N1CCn2ncnc2C1] | (R)-(5-chloro-7-(2-(hydroxymethyl)thieno[3,2- b]pyridin-7-yl)-2,3-dihydrobenzofuran-2- yl)(5,6-dihydro-[1,2,4]triazolo[1,5-a]pyrazin- 7(8H)-yl)methanone | |
| 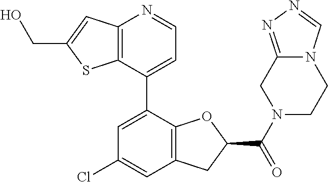 [SMILES: O=C([C@H]1Cc2cc(Cl)cc(-c3ccnc4cc(CO)sc34)c2O1)N1CCn2cnnc2C1] | (R)-(5-chloro-7-(2-(hydroxymethyl)thieno[3,2- b]pyridin-7-yl)-2,3-dihydrobenzofuran-2- yl)(5,6-dihydro-[1,2,4]triazolo[4,3-a]pyrazin- 7(8H)-yl)methanone | B |
| 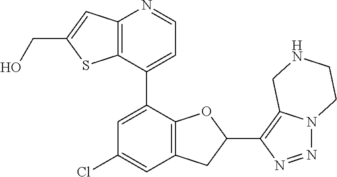 [SMILES: OCc1cc2nccc(-c3cc(Cl)cc4c3OC(c3nnn5c3CNCC5)C4)c2s1] | (7-(5-chloro-2-(4,5,6,7-tetrahydro- [1,2,3]triazolo[1,5-a]pyrazin-3-yl)-2,3- dihydrobenzofuran-7-yl)thieno[3,2-b]pyridin-2- yl)methanol | B |
| 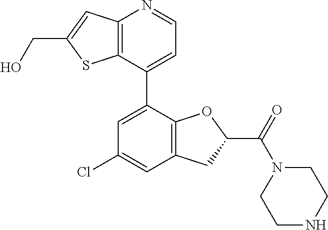 [SMILES: O=C([C@@H]1Cc2cc(Cl)cc(-c3ccnc4cc(CO)sc34)c2O1)N1CCNCC1] | (S)-(5-chloro-7-(2-(hydroxymethyl)thieno[3,2- b]pyridin-7-yl)-2,3-dihydrobenzofuran-2- yl)(piperazin-1-yl)methanone | B |
| 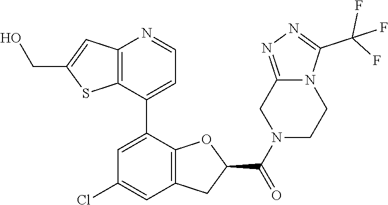 [SMILES: O=C([C@H]1Cc2cc(Cl)cc(-c3ccnc4cc(CO)sc34)c2O1)N1CCn2c(nnc2C(F)(F)F)C1] | (R)-(5-chloro-7-(2-(hydroxymethyl)thieno[3,2- b]pyridin-7-yl)-2,3-dihydrobenzofuran-2-yl)(3- (trifluoromethyl)-5,6-dihydro- [1,2,4]triazolo[4,3-a]pyrazin-7(8H)- yl)methanone | B |
| 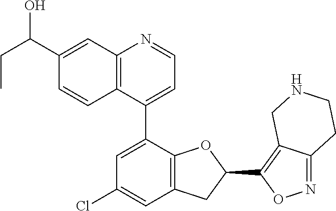 [SMILES: CCC(O)c1ccc2c(-c3cc(Cl)cc4c3O[C@@H](c3onc5c3CNCC5)C4)ccnc2c1] | 1-(4-((R)-5-chloro-2-(4,5,6,7- tetrahydroisoxazolo[4,3-c]pyridin-3-yl)-2,3- dihydrobenzofuran-7-yl)quinolin-7-yl)propan- 1-ol | B |
| 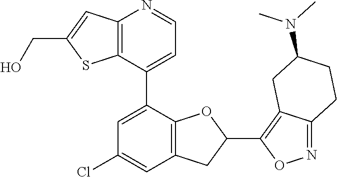 [SMILES: CN(C)[C@H]1CCc2noc(C3Cc4cc(Cl)cc(-c5ccnc6cc(CO)sc56)c4O3)c2C1] | (7-(5-chloro-2-((S)-5-(dimethylamino)-4,5,6,7- tetrahydrobenzo[c]isoxazol-3-yl)-2,3- dihydrobenzofuran-7-yl)thieno[3,2-b]pyridin-2- yl)methanol | B |
| 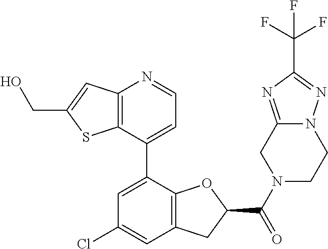 [SMILES: O=C([C@H]1Cc2cc(Cl)cc(-c3ccnc4cc(CO)sc34)c2O1)N1CCn2nc(C(F)(F)F)nc2C1] | (R)-(5-chloro-7-(2-(hydroxymethyl)thieno[3,2- b]pyridin-7-yl)-2,3-dihydrobenzofuran-2-yl)(2- (trifluoromethyl)-5,6-dihydro- [1,2,4]triazolo[1,5-a]pyrazin-7(8H)- yl)methanone | B |
| 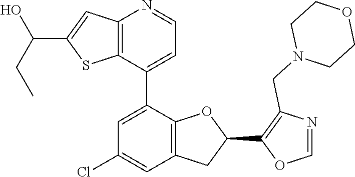 [SMILES: CCC(O)c1cc2nccc(-c3cc(Cl)cc4c3O[C@@H](c3ocnc3CN3CCOCC3)C4)c2s1] | 1-(7-((R)-5-chloro-2-(4- (morpholinomethyl)oxazol-5-yl)-2,3- dihydrobenzofuran-7-yl)thieno[3,2-b]pyridin-2- yl)propan-1-ol | B |
| 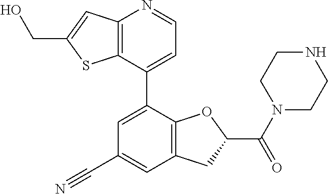 [SMILES: N#Cc1cc2c(c(-c3ccnc4cc(CO)sc34)c1)O[C@H](C(=O)N1CCNCC1)C2] | (R)-7-(2-(hydroxymethyl)thieno[3,2-b]pyridin- 7-yl)-2-(piperazine-1-carbonyl)-2,3- dihydrobenzofuran-5-carbonitrile | B |
| 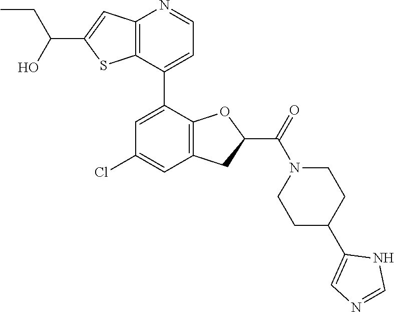 [SMILES: CCC(O)c1cc2nccc(-c3cc(Cl)cc4c3O[C@@H](C(=O)N3CCC(c5cnc[nH]5)CC3)C4)c2s1] | (4-(1H-imidazol-5-yl)piperidin-1-yl)((2R)-5- chloro-7-(2-(1-hydroxypropyl)thieno[3,2- b]pyridin-7-yl)-2,3-dihydrobenzofuran-2- yl)methanone | B |
| 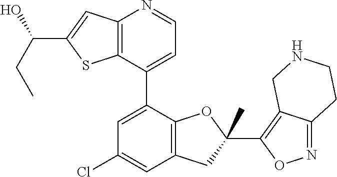 [SMILES: CC[C@H](O)c1cc2nccc(-c3cc(Cl)cc4c3O[C@@](C)(c3onc5c3CNCC5)C4)c2s1] | (S)-1-(7-((S)-5-chloro-2-methyl-2-(4,5,6,7- tetrahydroisoxazolo[4,3-c]pyridin-3-yl)-2,3- dihydrobenzofuran-7-yl)thieno[3,2-b]pyridin-2- yl)propan-1-ol | B |
| 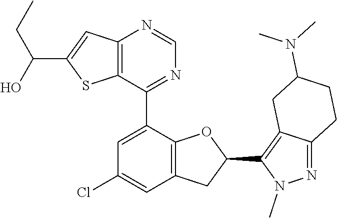 [SMILES: CCC(O)c1cc2ncnc(-c3cc(Cl)cc4c3O[C@@H](c3c5c(nn3C)CCC(N(C)C)C5)C4)c2s1] | 1-(4-((2R)-5-chloro-2-(5-(dimethylamino)-2- methyl-4,5,6,7-tetrahydro-2H-indazol-3-yl)-2,3- dihydrobenzofuran-7-yl)thieno[3,2- d]pyrimidin-6-yl)propan-1-ol | B |
| 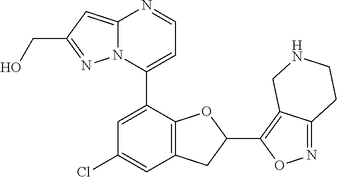 [SMILES: OCc1cc2nccc(-c3cc(Cl)cc4c3OC(c3onc5c3CNCC5)C4)n2n1] | (7-(5-chloro-2-(4,5,6,7- tetrahydroisoxazolo[4,3-c]pyridin-3-yl)-2,3- dihydrobenzofuran-7-yl)pyrazolo[1,5- a]pyrimidin-2-yl)methanol | B |
| 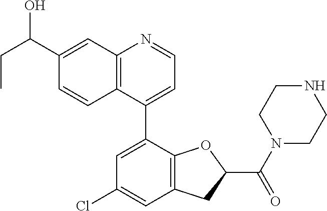 [SMILES: CCC(O)c1ccc2c(-c3cc(Cl)cc4c3O[C@@H](C(=O)N3CCNCC3)C4)ccnc2c1] | ((2R)-5-chloro-7-(7-(1- hydroxypropyl)quinolin-4-yl)-2,3- dihydrobenzofuran-2-yl)(piperazin-1- yl)methanone | B |
| 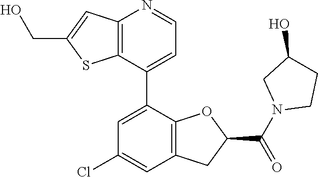 [SMILES: O=C([C@H]1Cc2cc(Cl)cc(-c3ccnc4cc(CO)sc34)c2O1)N1CC[C@H](O)C1] | ((R)-5-chloro-7-(2-(hydroxymethyl)thieno[3,2- b]pyridin-7-yl)-2,3-dihydrobenzofuran-2- yl)((S)-3-hydroxypyrrolidin-1-yl)methanone | C |
| 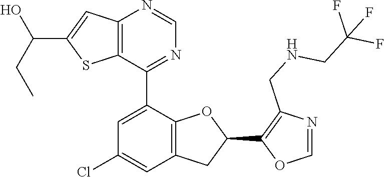 [SMILES: CCC(O)c1cc2ncnc(-c3cc(Cl)cc4c3O[C@@H](c3ocnc3CNCC(F)(F)F)C4)c2s1] | 1-(4-((R)-5-chloro-2-(4-(((2,2,2- trifluoroethyl)amino)methyl)oxazol-5-yl)-2,3- dihydrobenzofuran-7-yl)thieno[3,2- d]pyrimidin-6-yl)propan-1-ol | C |
| 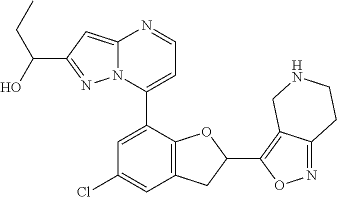 [SMILES: CCC(O)c1cc2nccc(-c3cc(Cl)cc4c3OC(c3onc5c3CNCC5)C4)n2n1] | 1-(7-(5-chloro-2-(4,5,6,7- tetrahydroisoxazolo[4,3-c]pyridin-3-yl)-2,3- dihydrobenzofuran-7-yl)pyrazolo[1,5- a]pyrimidin-2-yl)propan-1-ol | B |
| 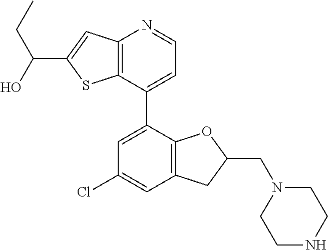 [SMILES: CCC(O)c1cc2nccc(-c3cc(Cl)cc4c3OC(CN3CCNCC3)C4)c2s1] | 1-(7-(5-chloro-2-(piperazin-1-ylmethyl)-2,3- dihydrobenzofuran-7-yl)thieno[3,2-b]pyridin-2- yl)propan-1-ol | C |
| 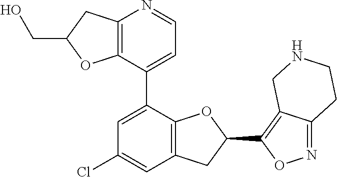 [SMILES: OCC1Cc2nccc(-c3cc(Cl)cc4c3O[C@@H](c3onc5c3CNCC5)C4)c2O1] | (7-((R)-5-chloro-2-(4,5,6,7- tetrahydroisoxazolo[4,3-c]pyridin-3-yl)-2,3- dihydrobenzofuran-7-yl)-2,3-dihydrofuro[3,2- b]pyridin-2-yl)methanol | C |
| 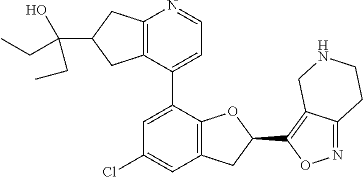 [SMILES: CCC(O)(CC)C1Cc2nccc(-c3cc(Cl)cc4c3O[C@@H](c3onc5c3CNCC5)C4)c2C1] | 3-(4-((R)-5-chloro-2-(4,5,6,7- tetrahydroisoxazolo[4,3-c]pyridin-3-yl)-2,3- dihydrobenzofuran-7-yl)-6,7-dihydro-5H- cyclopenta[b]pyridin-6-yl)pentan-3-ol | B |
| 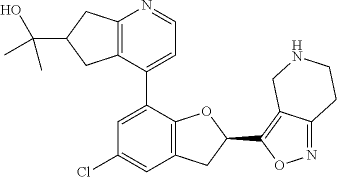 [SMILES: CC(C)(O)C1Cc2nccc(-c3cc(Cl)cc4c3O[C@@H](c3onc5c3CNCC5)C4)c2C1] | 2-(4-((R)-5-chloro-2-(4,5,6,7- tetrahydroisoxazolo[4,3-c]pyridin-3-yl)-2,3- dihydrobenzofuran-7-yl)-6,7-dihydro-5H- cyclopenta[b]pyridin-6-yl)propan-2-ol | B |
| 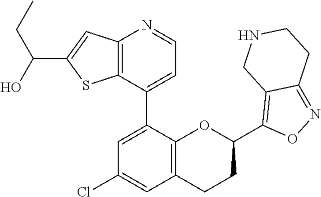 [SMILES: CCC(O)c1cc2nccc(-c3cc(Cl)cc4c3O[C@@H](c3onc5c3CNCC5)CC4)c2s1] | 1-(7-((R)-6-chloro-2-(4,5,6,7- tetrahydroisoxazolo[4,3-c]pyridin-3- yl)chroman-8-yl)thieno[3,2-b]pyridin-2- yl)propan-1-ol | A |
| 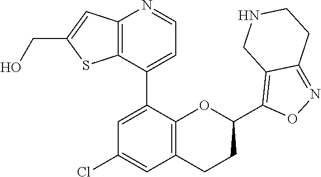 [SMILES: OCc1cc2nccc(-c3cc(Cl)cc4c3O[C@@H](c3onc5c3CNCC5)CC4)c2s1] | (R)-(7-(6-chloro-2-(4,5,6,7- tetrahydroisoxazolo[4,3-c]pyridin-3- yl)chroman-8-yl)thieno[3,2-b]pyridin-2- yl)methanol | A |
| 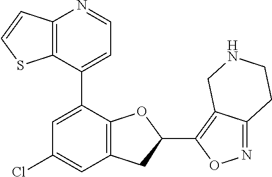 [SMILES: Clc1cc2c(c(-c3ccnc4ccsc34)c1)O[C@@H](c1onc3c1CNCC3)C2] | (R)-3-(5-chloro-7-(thieno[3,2-b]pyridin-7-yl)- 2,3-dihydrobenzofuran-2-yl)-4,5,6,7- tetrahydroisoxazolo[4,3-c]pyridine | A |
| 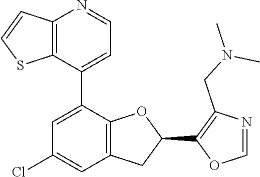 [SMILES: CN(C)Cc1ncoc1[C@H]1Cc2cc(Cl)cc(-c3ccnc4ccsc34)c2O1] | (R)-1-(5-(5-chloro-7-(thieno[3,2-b]pyridin-7- yl)-2,3-dihydrobenzofuran-2-yl)oxazol-4-yl)- N,N-dimethylmethanamine | A |
| 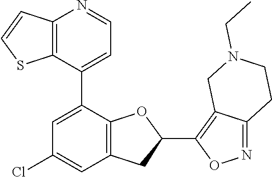 [SMILES: CCN1CCc2noc([C@H]3Cc4cc(Cl)cc(-c5ccnc6ccsc56)c4O3)c2C1] | (R)-3-(5-chloro-7-(thieno[3,2-b]pyridin-7-yl)- 2,3-dihydrobenzofuran-2-yl)-5-ethyl-4,5,6,7- tetrahydroisoxazolo[4,3-c]pyridine | A |
| 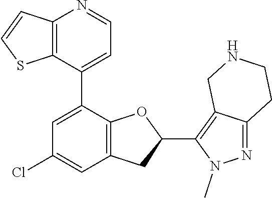 [SMILES: Cn1nc2c(c1[C@H]1Cc3cc(Cl)cc(-c4ccnc5ccsc45)c3O1)CNCC2] | (R)-7-(5-chloro-2-(2-methyl-4,5,6,7-tetrahydro- 2H-pyrazolo[4,3-c]pyridin-3-yl)-2,3- dihydrobenzofuran-7-yl)thieno[3,2-b]pyridine | A |
| 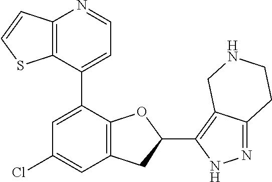 [SMILES: Clc1cc2c(c(-c3ccnc4ccsc34)c1)O[C@@H](c1[nH]nc3c1CNCC3)C2] | (R)-7-(5-chloro-2-(4,5,6,7-tetrahydro-2H- pyrazolo[4,3-c]pyridin-3-yl)-2,3- dihydrobenzofuran-7-yl)thieno[3,2-b]pyridine | A |
| 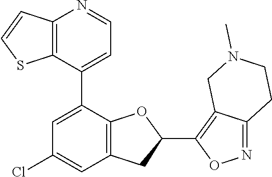 [SMILES: CN1CCc2noc([C@H]3Cc4cc(Cl)cc(-c5ccnc6ccsc56)c4O3)c2C1] | (R)-3-(5-chloro-7-(thieno[3,2-b]pyridin-7-yl)- 2,3-dihydrobenzofuran-2-yl)-5-methyl-4,5,6,7- tetrahydroisoxazolo[4,3-c]pyridine | A |
| 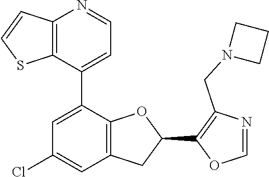 [SMILES: Clc1cc2c(c(-c3ccnc4ccsc34)c1)O[C@@H](c1ocnc1CN1CCC1)C2] | (R)-4-(azetidin-1-ylmethyl)-5-(5-chloro-7- (thieno[3,2-b]pyridin-7-yl)-2,3- dihydrobenzofuran-2-yl)oxazole | A |
| 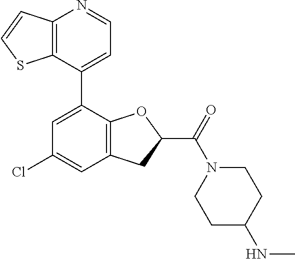 [SMILES: CNC1CCN(C(=O)[C@H]2Cc3cc(Cl)cc(-c4ccnc5ccsc45)c3O2)CC1] | (R)-(5-chloro-7-(thieno[3,2-b]pyridin-7-yl)-2,3- dihydrobenzofuran-2-yl)(4- (methylamino)piperidin-1-yl)methanone | A |
| 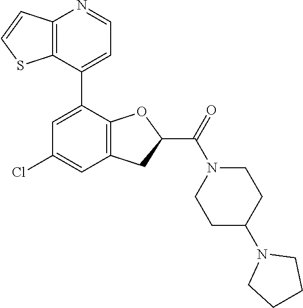 [SMILES: O=C([C@H]1Cc2cc(Cl)cc(-c3ccnc4ccsc34)c2O1)N1CCC(N2CCCC2)CC1] | (R)-(5-chloro-7-(thieno[3,2-b]pyridin-7-yl)-2,3- dihydrobenzofuran-2-yl)(4-(pyrrolidin-1- yl)piperidin-1-yl)methanone | A |
| 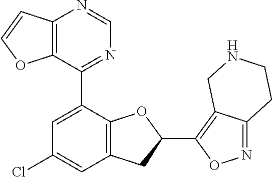 [SMILES: Clc1cc2c(c(-c3ncnc4ccoc34)c1)O[C@@H](c1onc3c1CNCC3)C2] | (R)-3-(5-chloro-7-(furo[3,2-d]pyrimidin-4-yl)- 2,3-dihydrobenzofuran-2-yl)-4,5,6,7- tetrahydroisoxazolo[4,3-c]pyridine | A |
| 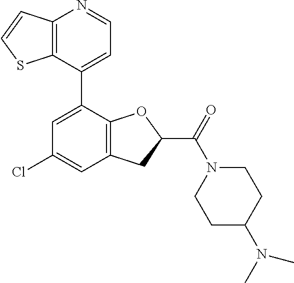 [SMILES: CN(C)C1CCN(C(=O)[C@H]2Cc3cc(Cl)cc(-c4ccnc5ccsc45)c3O2)CC1] | (R)-(5-chloro-7-(thieno[3,2-b]pyridin-7-yl)-2,3- dihydrobenzofuran-2-yl)(4- (dimethylamino)piperidin-1-yl)methanone | A |
| 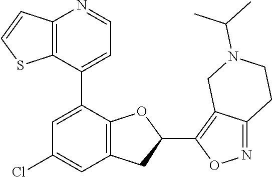 [SMILES: CC(C)N1CCc2noc([C@H]3Cc4cc(Cl)cc(-c5ccnc6ccsc56)c4O3)c2C1] | (R)-3-(5-chloro-7-(thieno[3,2-b]pyridin-7-yl)- 2,3-dihydrobenzofuran-2-yl)-5-isopropyl- 4,5,6,7-tetrahydroisoxazolo[4,3-c]pyridine | A |
| 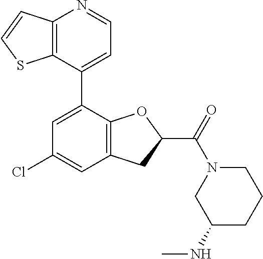 [SMILES: CN[C@H]1CCCN(C(=O)[C@H]2Cc3cc(Cl)cc(-c4ccnc5ccsc45)c3O2)C1] | ((R)-5-chloro-7-(thieno[3,2-b]pyridin-7-yl)-2,3- dihydrobenzofuran-2-yl)((S)-3- (methylamino)piperidin-1-yl)methanone | A |
| 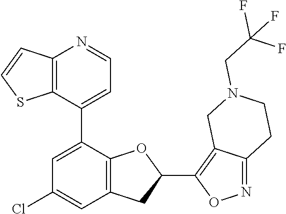 [SMILES: FC(F)(F)CN1CCc2noc([C@H]3Cc4cc(Cl)cc(-c5ccnc6ccsc56)c4O3)c2C1] | (R)-3-(5-chloro-7-(thieno[3,2-b]pyridin-7-yl)- 2,3-dihydrobenzofuran-2-yl)-5-(2,2,2- trifluoroethyl)-4,5,6,7-tetrahydroisoxazolo[4,3- c]pyridine | A |
| 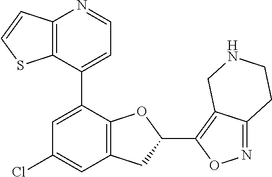 [SMILES: Clc1cc2c(c(-c3ccnc4ccsc34)c1)O[C@H](c1onc3c1CNCC3)C2] | (S)-3-(5-chloro-7-(thieno[3,2-b]pyridin-7-yl)- 2,3-dihydrobenzofuran-2-yl)-4,5,6,7- tetrahydroisoxazolo[4,3-c]pyridine | A |
| 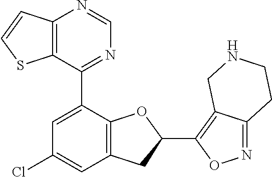 [SMILES: Clc1cc2c(c(-c3ncnc4ccsc34)c1)O[C@@H](c1onc3c1CNCC3)C2] | (R)-3-(5-chloro-7-(thieno[3,2-d]pyrimidin-4- yl)-2,3-dihydrobenzofuran-2-yl)-4,5,6,7- tetrahydroisoxazolo[4,3-c]pyridine | A |
| 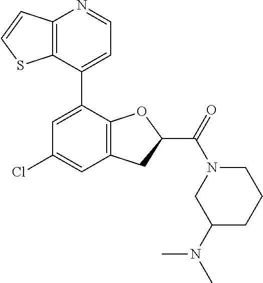 [SMILES: CN(C)C1CCCN(C(=O)[C@H]2Cc3cc(Cl)cc(-c4ccnc5ccsc45)c3O2)C1] | ((R)-5-chloro-7-(thieno[3,2-b]pyridin-7-yl)-2,3- dihydrobenzofuran-2-yl)(3- (dimethylamino)piperidin-1-yl)methanone | A |
| 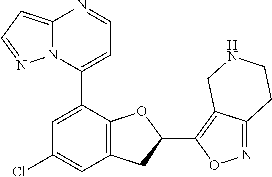 [SMILES: Clc1cc2c(c(-c3ccnc4ccnn34)c1)O[C@@H](c1onc3c1CNCC3)C2] | (R)-3-(5-chloro-7-(pyrazolo[1,5-a]pyrimidin-7- yl)-2,3-dihydrobenzofuran-2-yl)-4,5,6,7- tetrahydroisoxazolo[4,3-c]pyridine | A |
| 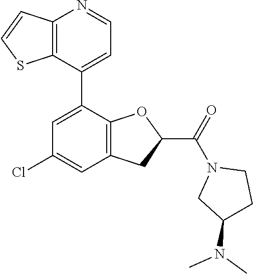 [SMILES: CN(C)[C@@H]1CCN(C(=O)[C@H]2Cc3cc(Cl)cc(-c4ccnc5ccsc45)c3O2)C1] | ((R)-5-chloro-7-(thieno[3,2-b]pyridin-7-yl)-2,3- dihydrobenzofuran-2-yl)((R)-3- (dimethylamino)pyrrolidin-1-yl)methanone | A |
| 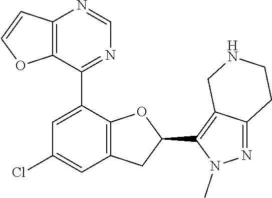 [SMILES: Cn1nc2c(c1[C@H]1Cc3cc(Cl)cc(-c4ncnc5ccoc45)c3O1)CNCC2] | (R)-4-(5-chloro-2-(2-methyl-4,5,6,7-tetrahydro- 2H-pyrazolo[4,3-c]pyridin-3-yl)-2,3- dihydrobenzofuran-7-yl)furo[3,2-d]pyrimidine | A |
| 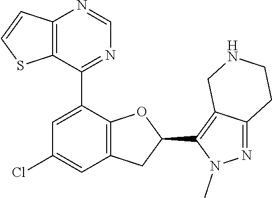 [SMILES: Cn1nc2c(c1[C@H]1Cc3cc(Cl)cc(-c4ncnc5ccsc45)c3O1)CNCC2] | (R)-4-(5-chloro-2-(2-methyl-4,5,6,7-tetrahydro- 2H-pyrazolo[4,3-c]pyridin-3-yl)-2,3- dihydrobenzofuran-7-yl)thieno[3,2- d]pyrimidine | A |
| 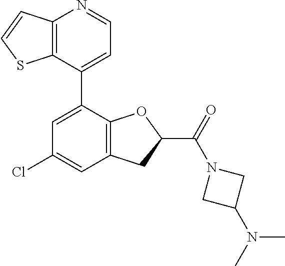 [SMILES: CN(C)C1CN(C(=O)[C@H]2Cc3cc(Cl)cc(-c4ccnc5ccsc45)c3O2)C1] | (R)-(5-chloro-7-(thieno[3,2-b]pyridin-7-yl)-2,3- dihydrobenzofuran-2-yl)(3- (dimethylamino)azetidin-1-yl)methanone | A |
| 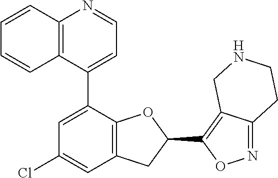 [SMILES: Clc1cc2c(c(-c3ccnc4ccccc34)c1)O[C@@H](c1onc3c1CNCC3)C2] | (R)-3-(5-chloro-7-(quinolin-4-yl)-2,3- dihydrobenzofuran-2-yl)-4,5,6,7- tetrahydroisoxazolo[4,3-c]pyridine | A |
| 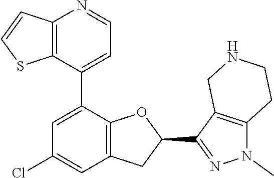 [SMILES: Cn1nc([C@H]2Cc3cc(Cl)cc(-c4ccnc5ccsc45)c3O2)c2c1CCNC2] | (R)-7-(5-chloro-2-(1-methyl-4,5,6,7-tetrahydro- 1H-pyrazolo[4,3-c]pyridin-3-yl)-2,3- dihydrobenzofuran-7-yl)thieno[3,2-b]pyridine | B |
| 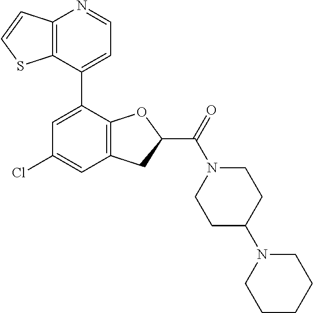 [SMILES: O=C([C@H]1Cc2cc(Cl)cc(-c3ccnc4ccsc34)c2O1)N1CCC(N2CCCCC2)CC1] | (R)-[1,4′-bipiperidin]-1′-yl(5-chloro-7- (thieno[3,2-b]pyridin-7-yl)-2,3- dihydrobenzofuran-2-yl)methanone | B |
| 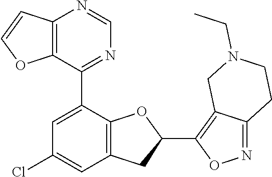 [SMILES: CCN1CCc2noc([C@H]3Cc4cc(Cl)cc(-c5ncnc6ccoc56)c4O3)c2C1] | (R)-3-(5-chloro-7-(furo[3,2-d]pyrimidin-4-yl)- 2,3-dihydrobenzofuran-2-yl)-5-ethyl-4,5,6,7- tetrahydroisoxazolo[4,3-c]pyridine | B |
| 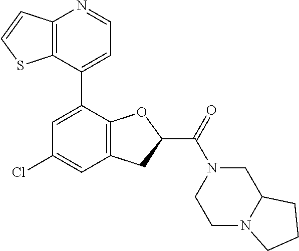 [SMILES: O=C([C@H]1Cc2cc(Cl)cc(-c3ccnc4ccsc34)c2O1)N1CCN2CCCC2C1] | ((R)-5-chloro-7-(thieno[3,2-b]pyridin-7-yl)-2,3- dihydrobenzofuran-2- yl)(hexahydropyrrolo[1,2-a]pyrazin-2(1H)- yl)methanone | B |
| 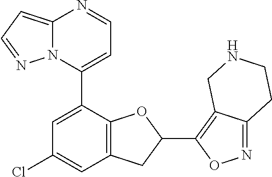 [SMILES: Clc1cc2c(c(-c3ccnc4ccnn34)c1)OC(c1onc3c1CNCC3)C2] | 3-(5-chloro-7-(pyrazolo[1,5-a]pyrimidin-7-yl)- 2,3-dihydrobenzofuran-2-yl)-4,5,6,7- tetrahydroisoxazolo[4,3-c]pyridine | B |
| 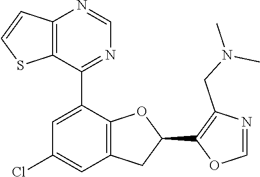 [SMILES: CN(C)Cc1ncoc1[C@H]1Cc2cc(Cl)cc(-c3ncnc4ccsc34)c2O1] | (R)-1-(5-(5-chloro-7-(thieno[3,2-d]pyrimidin-4- yl)-2,3-dihydrobenzofuran-2-yl)oxazol-4-yl)- N,N-dimethylmethanamine | B |
| 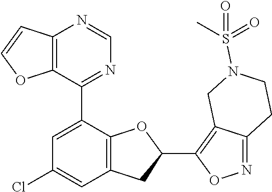 [SMILES: CS(=O)(=O)N1CCc2noc([C@H]3Cc4cc(Cl)cc(-c5ncnc6ccoc56)c4O3)c2C1] | (R)-3-(5-chloro-7-(furo[3,2-d]pyrimidin-4-yl)- 2,3-dihydrobenzofuran-2-yl)-5- (methylsulfonyl)-4,5,6,7- tetrahydroisoxazolo[4,3-c]pyridine | B |
| 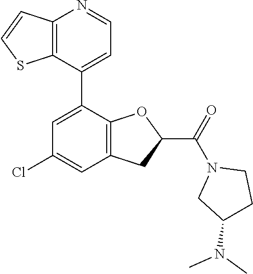 [SMILES: CN(C)[C@H]1CCN(C(=O)[C@H]2Cc3cc(Cl)cc(-c4ccnc5ccsc45)c3O2)C1] | ((R)-5-chloro-7-(thieno[3,2-b]pyridin-7-yl)-2,3- dihydrobenzofuran-2-yl)((S)-3- (dimethylamino)pyrrolidin-1-yl)methanone | B |
| 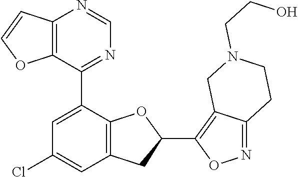 [SMILES: OCCN1CCc2noc([C@H]3Cc4cc(Cl)cc(-c5ncnc6ccoc56)c4O3)c2C1] | (R)-2-(3-(5-chloro-7-(furo[3,2-d]pyrimidin-4- yl)-2,3-dihydrobenzofuran-2-yl)-6,7- dihydroisoxazolo[4,3-c]pyridin-5(4H)-yl)ethan- 1-ol | B |
| 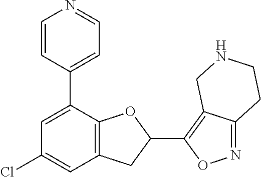 [SMILES: Clc1cc2c(c(-c3ccncc3)c1)OC(c1onc3c1CNCC3)C2] | 3-(5-chloro-7-(pyridin-4-yl)-2,3- dihydrobenzofuran-2-yl)-4,5,6,7- tetrahydroisoxazolo[4,3-c]pyridine | B |
| 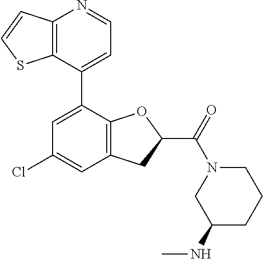 [SMILES: CN[C@@H]1CCCN(C(=O)[C@H]2Cc3cc(Cl)cc(-c4ccnc5ccsc45)c3O2)C1] | ((R)-5-chloro-7-(thieno[3,2-b]pyridin-7-yl)-2,3- dihydrobenzofuran-2-yl)((R)-3- (methylamino)piperidin-1-yl)methanone | C |
| 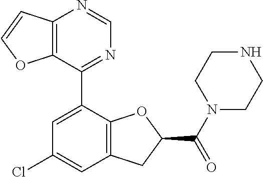 [SMILES: O=C([C@H]1Cc2cc(Cl)cc(-c3ncnc4ccoc34)c2O1)N1CCNCC1] | (R)-(5-chloro-7-(furo[3,2-d]pyrimidin-4-yl)- 2,3-dihydrobenzofuran-2-yl)(piperazin-1- yl)methanone | C |
| 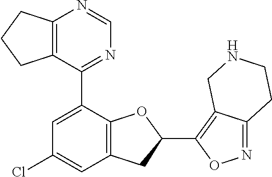 [SMILES: Clc1cc2c(c(-c3ncnc4c3CCC4)c1)O[C@@H](c1onc3c1CNCC3)C2] | (R)-3-(5-chloro-7-(6,7-dihydro-5H- cyclopenta[d]pyrimidin-4-yl)-2,3- dihydrobenzofuran-2-yl)-4,5,6,7- tetrahydroisoxazolo[4,3-c]pyridine | C |
| 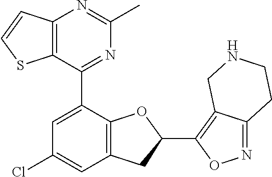 [SMILES: Cc1nc(-c2cc(Cl)cc3c2O[C@@H](c2onc4c2CNCC4)C3)c2sccc2n1] | (R)-3-(5-chloro-7-(2-methylthieno[3,2- d]pyrimidin-4-yl)-2,3-dihydrobenzofuran-2-yl)- 4,5,6,7-tetrahydroisoxazolo[4,3-c]pyridine | C |
| 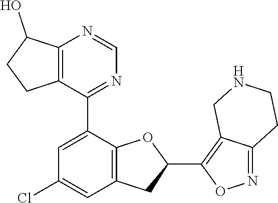 [SMILES: OC1CCc2c(-c3cc(Cl)cc4c3O[C@@H](c3onc5c3CNCC5)C4)ncnc21] | 4-((R)-5-chloro-2-(4,5,6,7- tetrahydroisoxazolo[4,3-c]pyridin-3-yl)-2,3- dihydrobenzofuran-7-yl)-6,7-dihydro-5H- cyclopenta[b]pyridin-7-ol | B |
| 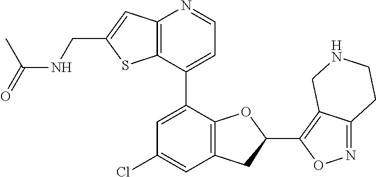 [SMILES: CC(=O)NCc1cc2nccc(-c3cc(Cl)cc4c3O[C@@H](c3onc5c3CNCC5)C4)c2s1] | (R)-N-((7-(5-chloro-2-(4,5,6,7- tetrahydroisoxazolo[4,3-c]pyridin-3-yl)-2,3- dihydrobenzofuran-7-yl)thieno[3,2-b]pyridin-2- yl)methyl)acetamide | A |
| 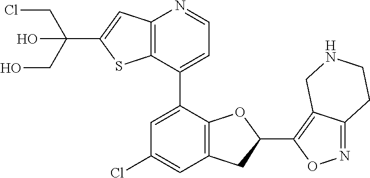 [SMILES: OCC(O)(CCl)c1cc2nccc(-c3cc(Cl)cc4c3O[C@@H](c3onc5c3CNCC5)C4)c2s1] | 3-chloro-2-(7-((R)-5-chloro-2-(4,5,6,7- tetrahydroisoxazolo[4,3-c]pyridin-3-yl)-2,3- dihydrobenzofuran-7-yl)thieno[3,2-b]pyridin-2- yl)propane-1,2-diol | A |
| 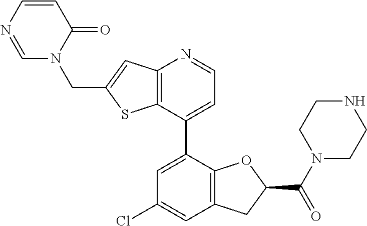 [SMILES: O=C([C@H]1Cc2cc(Cl)cc(-c3ccnc4cc(Cn5cnccc5=O)sc34)c2O1)N1CCNCC1] | (R)-3-((7-(5-chloro-2-(piperazine-1-carbonyl)- 2,3-dihydrobenzofuran-7-yl)thieno[3,2- b]pyridin-2-yl)methyl)pyrimidin-4(3H)-one | A |
| 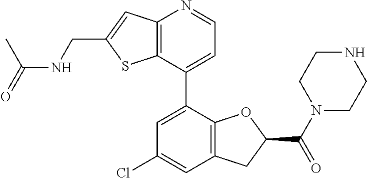 [SMILES: CC(=O)NCc1cc2nccc(-c3cc(Cl)cc4c3O[C@@H](C(=O)N3CCNCC3)C4)c2s1] | (R)-N-((7-(5-chloro-2-(piperazine-1-carbonyl)- 2,3-dihydrobenzofuran-7-yl)thieno[3,2- b]pyridin-2-yl)methyl)acetamide | A |
| 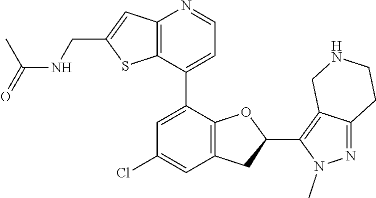 [SMILES: CC(=O)NCc1cc2nccc(-c3cc(Cl)cc4c3O[C@@H](c3c5c(nn3C)CCNC5)C4)c2s1] | (R)-N-((7-(5-chloro-2-(2-methyl-4,5,6,7- tetrahydro-2H-pyrazolo[4,3-c]pyridin-3-yl)- 2,3-dihydrobenzofuran-7-yl)thieno[3,2- b]pyridin-2-yl)methyl)acetamide | A |
| 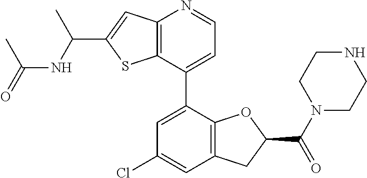 [SMILES: CC(=O)NC(C)c1cc2nccc(-c3cc(Cl)cc4c3O[C@@H](C(=O)N3CCNCC3)C4)c2s1] | N-(1-(7-((R)-5-chloro-2-(piperazine-1- carbonyl)-2,3-dihydrobenzofuran-7- yl)thieno[3,2-b]pyridin-2-yl)ethyl)acetamide | A |
| 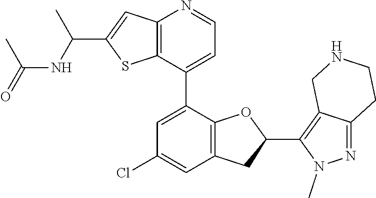 [SMILES: CC(=O)NC(C)c1cc2nccc(-c3cc(Cl)cc4c3O[C@@H](c3c5c(nn3C)CCNC5)C4)c2s1] | N-(1-(7-((R)-5-chloro-2-(2-methyl-4,5,6,7- tetrahydro-2H-pyrazolo[4,3-c]pyridin-3-yl)- 2,3-dihydrobenzofuran-7-yl)thieno[3,2- b]pyridin-2-yl)ethyl)acetamide | A |
| 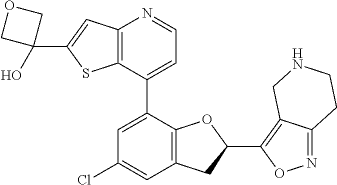 [SMILES: OC1(c2cc3nccc(-c4cc(Cl)cc5c4O[C@@H](c4onc6c4CNCC6)C5)c3s2)COC1] | (R)-3-(7-(5-chloro-2-(4,5,6,7- tetrahydroisoxazolo[4,3-c]pyridin-3-yl)-2,3- dihydrobenzofuran-7-yl)thieno[3,2-b]pyridin-2- yl)oxetan-3-ol | A |
| 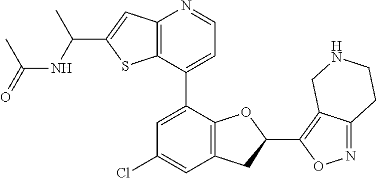 [SMILES: CC(=O)NC(C)c1cc2nccc(-c3cc(Cl)cc4c3O[C@@H](c3onc5c3CNCC5)C4)c2s1] | N-(1-(7-((R)-5-chloro-2-(4,5,6,7- tetrahydroisoxazolo[4,3-c]pyridin-3-yl)-2,3- dihydrobenzofuran-7-yl)thieno[3,2-b]pyridin-2- yl)ethyl)acetamide | A |
| 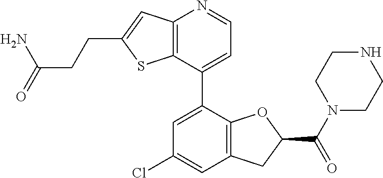 [SMILES: NC(=O)CCc1cc2nccc(-c3cc(Cl)cc4c3O[C@@H](C(=O)N3CCNCC3)C4)c2s1] | (R)-3-(7-(5-chloro-2-(piperazine-1-carbonyl)- 2,3-dihydrobenzofuran-7-yl)thieno[3,2- b]pyridin-2-yl)propenamide | A |
| 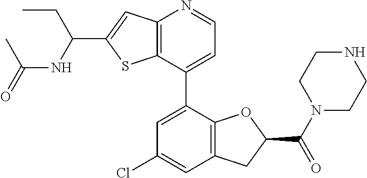 [SMILES: CCC(NC(C)=O)c1cc2nccc(-c3cc(Cl)cc4c3O[C@@H](C(=O)N3CCNCC3)C4)c2s1] | N-(1-(7-((R)-5-chloro-2-(piperazine-1- carbonyl)-2,3-dihydrobenzofuran-7- yl)thieno[3,2-b]pyridin-2-yl)propyl)acetamide | A |
| 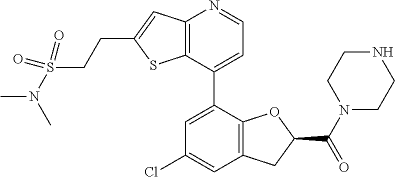 [SMILES: CN(C)S(=O)(=O)CCc1cc2nccc(-c3cc(Cl)cc4c3O[C@@H](C(=O)N3CCNCC3)C4)c2s1] | (R)-2-(7-(5-chloro-2-(piperazine-1-carbonyl)- 2,3-dihydrobenzofuran-7-yl)thieno[3,2- b]pyridin-2-yl)-N,N-dimethylethane-1- sulfonamide | A |
| 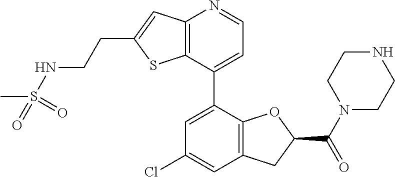 [SMILES: CS(=O)(=O)NCCc1cc2nccc(-c3cc(Cl)cc4c3O[C@@H](C(=O)N3CCNCC3)C4)c2s1] | (R)-N-(2-(7-(5-chloro-2-(piperazine-1- carbonyl)-2,3-dihydrobenzofuran-7- yl)thieno[3,2-b]pyridin-2- yl)ethyl)methanesulfonamide | A |
| 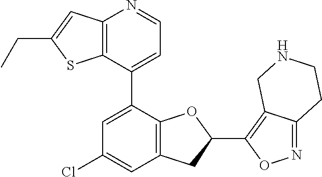 [SMILES: CCc1cc2nccc(-c3cc(Cl)cc4c3O[C@@H](c3onc5c3CNCC5)C4)c2s1] | (R)-3-(5-chloro-7-(2-ethylthieno[3,2-b]pyridin- 7-yl)-2,3-dihydrobenzofuran-2-yl)-4,5,6,7- tetrahydroisoxazolo[4,3-c]pyridine | A |
| 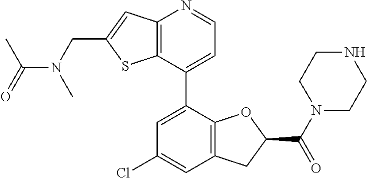 [SMILES: CC(=O)N(C)Cc1cc2nccc(-c3cc(Cl)cc4c3O[C@@H](C(=O)N3CCNCC3)C4)c2s1] | (R)-N-((7-(5-chloro-2-(piperazine-1-carbonyl)- 2,3-dihydrobenzofuran-7-yl)thieno[3,2- b]pyridin-2-yl)methyl)-N-methylacetamide | A |
| 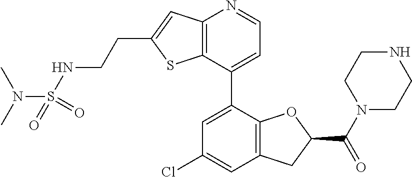 [SMILES: CN(C)S(=O)(=O)NCCc1cc2nccc(-c3cc(Cl)cc4c3O[C@@H](C(=O)N3CCNCC3)C4)c2s1] | (R)-N-(2-(7-(5-chloro-2-(piperazine-1- carbonyl)-2,3-dihydrobenzofuran-7- yl)thieno[3,2-b]pyridin-2- yl)ethyl)dimethylamimosulfonylurea | A |
| 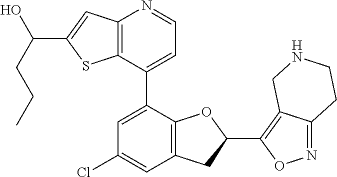 [SMILES: CCCC(O)c1cc2nccc(-c3cc(Cl)cc4c3O[C@@H](c3onc5c3CNCC5)C4)c2s1] | 1-(7-((R)-5-chloro-2-(4,5,6,7- tetrahydroisoxazolo[4,3-c]pyridin-3-yl)-2,3- dihydrobenzofuran-7-yl)thieno[3,2-b]pyridin-2- yl)butan-1-ol | A |
| 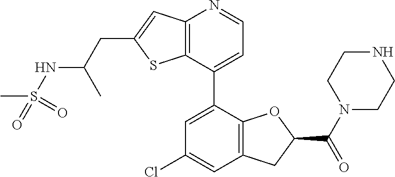 [SMILES: CC(Cc1cc2nccc(-c3cc(Cl)cc4c3O[C@@H](C(=O)N3CCNCC3)C4)c2s1)NS(C)(=O)=O] | N-(1-(7-((R)-5-chloro-2-(piperazine-1- carbonyl)-2,3-dihydrobenzofuran-7- yl)thieno[3,2-b]pyridin-2-yl)propan-2- yl)methanesulfonamide | A |
| 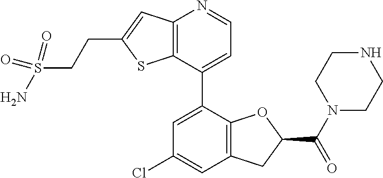 [SMILES: NS(=O)(=O)CCc1cc2nccc(-c3cc(Cl)cc4c3O[C@@H](C(=O)N3CCNCC3)C4)c2s1] | (R)-2-(7-(5-chloro-2-(piperazine-1-carbonyl)- 2,3-dihydrobenzofuran-7-yl)thieno[3,2- b]pyridin-2-yl)ethane-1-sulfonamide | A |
| 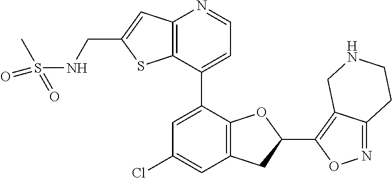 [SMILES: CS(=O)(=O)NCc1cc2nccc(-c3cc(Cl)cc4c3O[C@@H](c3onc5c3CNCC5)C4)c2s1] | (R)-N-((7-(5-chloro-2-(4,5,6,7- tetrahydroisoxazolo[4,3-c]pyridin-3-yl)-2,3- dihydrobenzofuran-7-yl)thieno[3,2-b]pyridin-2- yl)methyl)methanesulfonamide | A |
| 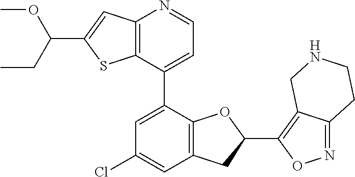 [SMILES: CCC(OC)c1cc2nccc(-c3cc(Cl)cc4c3O[C@@H](c3onc5c3CNCC5)C4)c2s1] | 3-((2R)-5-chloro-7-(2-(1- methoxypropyl)thieno[3,2-b]pyridin-7-yl)-2,3- dihydrobenzofuran-2-yl)-4,5,6,7- tetrahydroisoxazolo[4,3-c]pyridine | A |
| 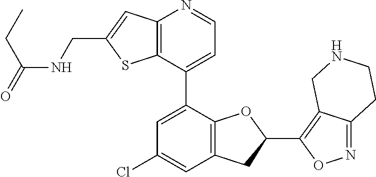 [SMILES: CCC(=O)NCc1cc2nccc(-c3cc(Cl)cc4c3O[C@@H](c3onc5c3CNCC5)C4)c2s1] | (R)-N-((7-(5-chloro-2-(4,5,6,7- tetrahydroisoxazolo[4,3-c]pyridin-3-yl)-2,3- dihydrobenzofuran-7-yl)thieno[3,2-b]pyridin-2- yl)methyl)propionamide | A |
| 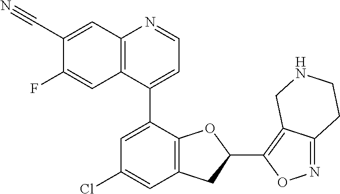 [SMILES: N#Cc1cc2nccc(-c3cc(Cl)cc4c3O[C@@H](c3onc5c3CNCC5)C4)c2cc1F] | (R)-4-(5-chloro-2-(4,5,6,7- tetrahydroisoxazolo[4,3-c]pyridin-3-yl)-2,3- dihydrobenzofuran-7-yl)-6-fluoroquinoline-7- carbonitrile | A |
| 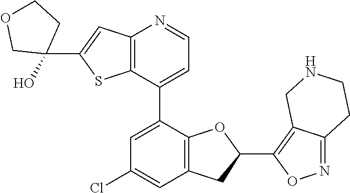 [SMILES: O[C@@]1(c2cc3nccc(-c4cc(Cl)cc5c4O[C@@H](c4onc6c4CNCC6)C5)c3s2)CCOC1] | (S)-3-(7-((R)-5-chloro-2-(4,5,6,7- tetrahydroisoxazolo[4,3-c]pyridin-3-yl)-2,3- dihydrobenzofuran-7-yl)thieno[3,2-b]pyridin-2- yl)tetrahydrofuran-3-ol | A |
| 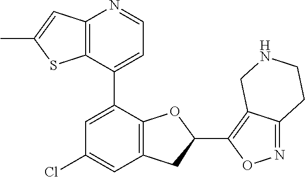 [SMILES: Cc1cc2nccc(-c3cc(Cl)cc4c3O[C@@H](c3onc5c3CNCC5)C4)c2s1] | (R)-3-(5-chloro-7-(2-methylthieno[3,2- b]pyridin-7-yl)-2,3-dihydrobenzofuran-2-yl)- 4,5,6,7-tetrahydroisoxazolo[4,3-c]pyridine | A |
| 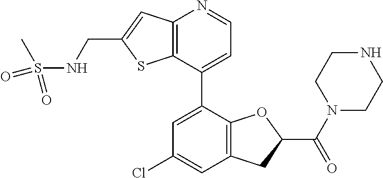 [SMILES: CS(=O)(=O)NCc1cc2nccc(-c3cc(Cl)cc4c3O[C@@H](C(=O)N3CCNCC3)C4)c2s1] | (R)-N-((7-(5-chloro-2-(piperazine-1-carbonyl)- 2,3-dihydrobenzofuran-7-yl)thieno[3,2- b]pyridin-2-yl)methyl)methanesulfonamide | A |
| 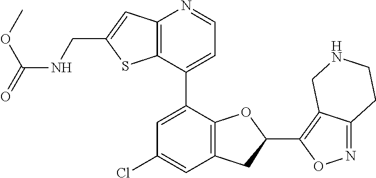 [SMILES: COC(=O)NCc1cc2nccc(-c3cc(Cl)cc4c3O[C@@H](c3onc5c3CNCC5)C4)c2s1] | Methyl (R)-((7-(5-chloro-2-(4,5,6,7- tetrahydroisoxazolo[4,3-c]pyridin-3-yl)-2,3- dihydrobenzofuran-7-yl)thieno[3,2-b]pyridin-2- yl)methyl)carbamate | A |
| 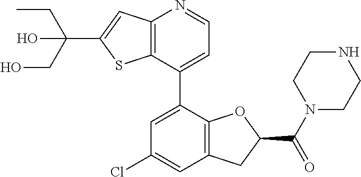 [SMILES: CCC(O)(CO)c1cc2nccc(-c3cc(Cl)cc4c3O[C@@H](C(=O)N3CCNCC3)C4)c2s1] | ((2R)-5-chloro-7-(2-(1,2-dihydroxybutan-2- yl)thieno[3,2-b]pyridin-7-yl)-2,3- dihydrobenzofuran-2-yl)(piperazin-1- yl)methanone | A |
| 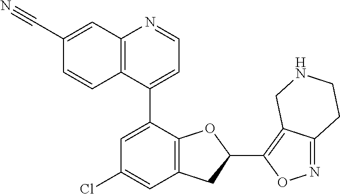 [SMILES: N#Cc1ccc2c(-c3cc(Cl)cc4c3O[C@@H](c3onc5c3CNCC5)C4)ccnc2c1] | (R)-4-(5-chloro-2-(4,5,6,7- tetrahydroisoxazolo[4,3-c]pyridin-3-yl)-2,3- dihydrobenzofuran-7-yl)quinoline-7- carbonitrile | A |
| 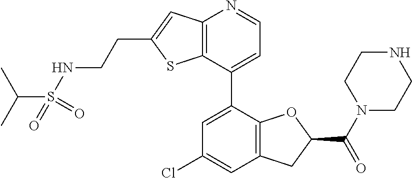 [SMILES: CC(C)S(=O)(=O)NCCc1cc2nccc(-c3cc(Cl)cc4c3O[C@@H](C(=O)N3CCNCC3)C4)c2s1] | (R)-N-(2-(7-(5-chloro-2-(piperazine-1- carbonyl)-2,3-dihydrobenzofuran-7- yl)thieno[3,2-b]pyridin-2-yl)ethyl)propane-2- sulfonamide | A |
| 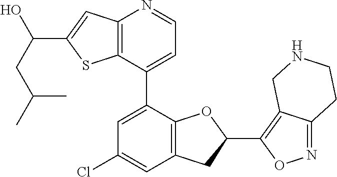 [SMILES: CC(C)CC(O)c1cc2nccc(-c3cc(Cl)cc4c3O[C@@H](c3onc5c3CNCC5)C4)c2s1] | 1-(7-((R)-5-chloro-2-(4,5,6,7- tetrahydroisoxazolo[4,3-c]pyridin-3-yl)-2,3- dihydrobenzofuran-7-yl)thieno[3,2-b]pyridin-2- yl)-3-methylbutan-1-ol | A |
| 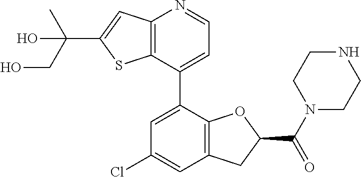 [SMILES: CC(O)(CO)c1cc2nccc(-c3cc(Cl)cc4c3O[C@@H](C(=O)N3CCNCC3)C4)c2s1] | ((2R)-5-chloro-7-(2-(1,2-dihydroxypropan-2- yl)thieno[3,2-b]pyridin-7-yl)-2,3- dihydrobenzofuran-2-yl)(piperazin-1- yl)methanone | A |
| 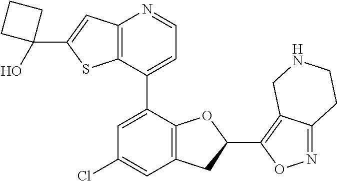 [SMILES: OC1(c2cc3nccc(-c4cc(Cl)cc5c4O[C@@H](c4onc6c4CNCC6)C5)c3s2)CCC1] | (R)-1-(7-(5-chloro-2-(4,5,6,7- tetrahydroisoxazolo[4,3-c]pyridin-3-yl)-2,3- dihydrobenzofuran-7-yl)thieno[3,2-b]pyridin-2- yl)cyclobutan-1-ol | A |
| 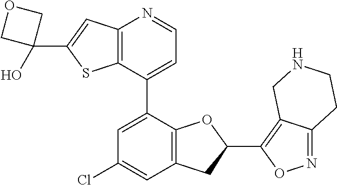 [SMILES: OC1(c2cc3nccc(-c4cc(Cl)cc5c4O[C@@H](c4onc6c4CNCC6)C5)c3s2)COC1] | (R)-3-(4-(5-chloro-2-(4,5,6,7- tetrahydroisoxazolo[4,3-c]pyridin-3-yl)-2,3- dihydrobenzofuran-7-yl)thieno[3,2- d]pyrimidin-6-yl)oxetan-3-ol | A |
| 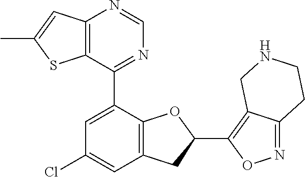 [SMILES: Cc1cc2ncnc(-c3cc(Cl)cc4c3O[C@@H](c3onc5c3CNCC5)C4)c2s1] | (R)-3-(5-chloro-7-(6-methylthieno[3,2- d]pyrimidin-4-yl)-2,3-dihydrobenzofuran-2-yl)- 4,5,6,7-tetrahydroisoxazolo[4,3-c]pyridine | A |
| 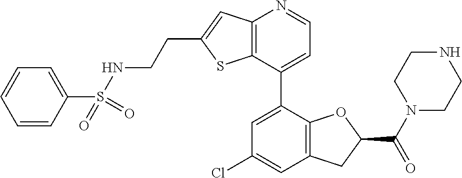 [SMILES: O=C([C@H]1Cc2cc(Cl)cc(-c3ccnc4cc(CCNS(=O)(=O)c5ccccc5)sc34)c2O1)N1CCNCC1] | (R)-N-(2-(7-(5-chloro-2-(piperazine-1- carbonyl)-2,3-dihydrobenzofuran-7- yl)thieno[3,2-b]pyridin-2- yl)ethyl)benzenesulfonamide | A |
| 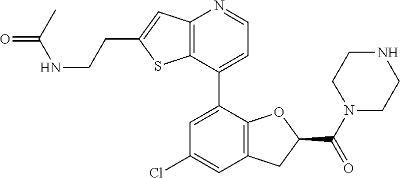 [SMILES: CC(=O)NCCc1cc2nccc(-c3cc(Cl)cc4c3O[C@@H](C(=O)N3CCNCC3)C4)c2s1] | (R)-N-(2-(7-(5-chloro-2-(piperazine-1- carbonyl)-2,3-dihydrobenzofuran-7- yl)thieno[3,2-b]pyridin-2-yl)ethyl)acetamide | A |
| 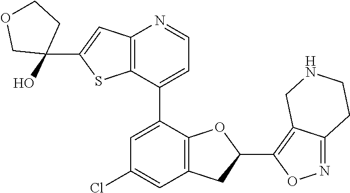 [SMILES: O[C@]1(c2cc3nccc(-c4cc(Cl)cc5c4O[C@@H](c4onc6c4CNCC6)C5)c3s2)CCOC1] | (R)-3-(7-((R)-5-chloro-2-(4,5,6,7- tetrahydroisoxazolo[4,3-c]pyridin-3-yl)-2,3- dihydrobenzofuran-7-yl)thieno[3,2-b]pyridin-2- yl)tetrahydrofuran-3-ol | A |
| 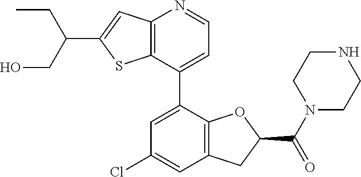 [SMILES: CCC(CO)c1cc2nccc(-c3cc(Cl)cc4c3O[C@@H](C(=O)N3CCNCC3)C4)c2s1] | ((2R)-5-chloro-7-(2-(1-hydroxybutan-2- yl)thieno[3,2-b]pyridin-7-yl)-2,3- dihydrobenzofuran-2-yl)(piperazin-1- yl)methanone | A |
| 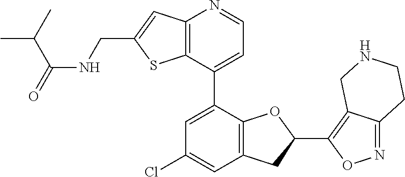 [SMILES: CC(C)C(=O)NCc1cc2nccc(-c3cc(Cl)cc4c3O[C@@H](c3onc5c3CNCC5)C4)c2s1] | (R)-N-((7-(5-chloro-2-(4,5,6,7- tetrahydroisoxazolo[4,3-c]pyridin-3-yl)-2,3- dihydrobenzofuran-7-yl)thieno[3,2-b]pyridin-2- yl)methyl)isobutyramide | A |
| 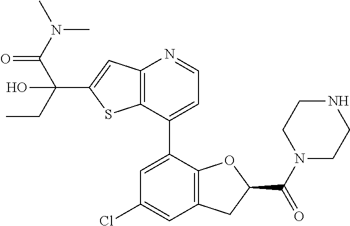 [SMILES: CCC(O)(C(=O)N(C)C)c1cc2nccc(-c3cc(Cl)cc4c3O[C@@H](C(=O)N3CCNCC3)C4)c2s1] | 2-(7-((R)-5-chloro-2-(piperazine-1-carbonyl)- 2,3-dihydrobenzofuran-7-yl)thieno[3,2- b]pyridin-2-yl)-2-hydroxy-N,N- dimethylbutanamide | A |
| 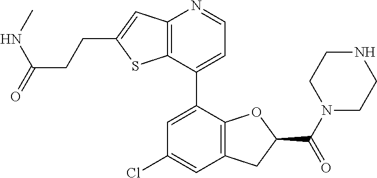 [SMILES: CNC(=O)CCc1cc2nccc(-c3cc(Cl)cc4c3O[C@@H](C(=O)N3CCNCC3)C4)c2s1] | (R)-3-(7-(5-chloro-2-(piperazine-1-carbonyl)- 2,3-dihydrobenzofuran-7-yl)thieno[3,2- b]pyridin-2-yl)-N-methylpropanamide | A |
| 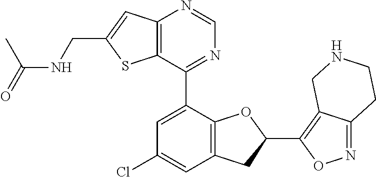 [SMILES: CC(=O)NCc1cc2ncnc(-c3cc(Cl)cc4c3O[C@@H](c3onc5c3CNCC5)C4)c2s1] | (R)-N-((4-(5-chloro-2-(4,5,6,7- tetrahydroisoxazolo[4,3-c]pyridin-3-yl)-2,3- dihydrobenzofuran-7-yl)thieno[3,2- d]pyrimidin-6-yl)methyl)acetamide | A |
| 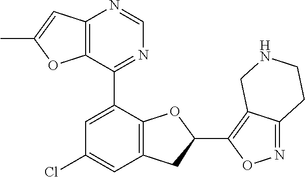 [SMILES: Cc1cc2ncnc(-c3cc(Cl)cc4c3O[C@@H](c3onc5c3CNCC5)C4)c2o1] | (R)-3-(5-chloro-7-(6-methylfuro[3,2- d]pyrimidin-4-yl)-2,3-dihydrobenzofuran-2-yl)- 4,5,6,7-tetrahydroisoxazolo[4,3-c]pyridine | A |
| 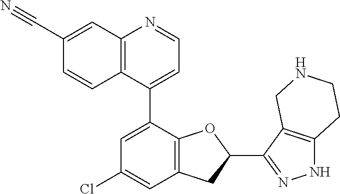 [SMILES: N#Cc1ccc2c(-c3cc(Cl)cc4c3O[C@@H](c3n[nH]c5c3CNCC5)C4)ccnc2c1] | (R)-4-(5-chloro-2-(4,5,6,7-tetrahydro-1H- pyrazolo[4,3-c]pyridin-3-yl)-2,3- dihydrobenzofuran-7-yl)quinoline-7- carbonitrile | A |
| 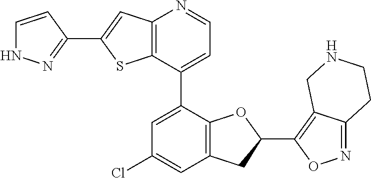 [SMILES: Clc1cc2c(c(-c3ccnc4cc(-c5cc[nH]n5)sc34)c1)O[C@@H](c1onc3c1CNCC3)C2] | (R)-3-(7-(2-(1H-pyrazol-3-yl)thieno[3,2- b]pyridin-7-yl)-5-chloro-2,3- dihydrobenzofuran-2-yl)-4,5,6,7- tetrahydroisoxazolo[4,3-c]pyridine | A |
| 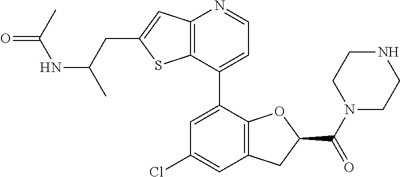 [SMILES: CC(=O)NC(C)Cc1cc2nccc(-c3cc(Cl)cc4c3O[C@@H](C(=O)N3CCNCC3)C4)c2s1] | N-(1-(7-((R)-5-chloro-2-(piperazine-1- carbonyl)-2,3-dihydrobenzofuran-7- yl)thieno[3,2-b]pyridin-2-yl)propan-2- yl)acetamide | A |
| 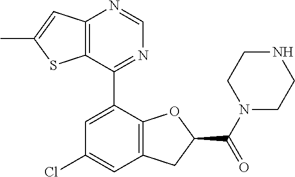 [SMILES: Cc1cc2ncnc(-c3cc(Cl)cc4c3O[C@@H](C(=O)N3CCNCC3)C4)c2s1] | (R)-(5-chloro-7-(6-methylthieno[3,2- d]pyrimidin-4-yl)-2,3-dihydrobenzofuran-2- yl)(piperazin-1-yl)methanone | A |
| 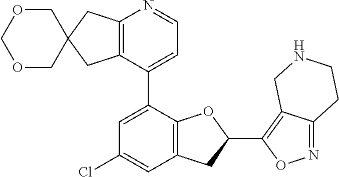 [SMILES: Clc1cc2c(c(-c3ccnc4c3CC3(COCOC3)C4)c1)O[C@@H](c1onc3c1CNCC3)C2] | (R)-4-(5-chloro-2-(4,5,6,7- tetrahydroisoxazolo[4,3-c]pyridin-3-yl)-2,3- dihydrobenzofuran-7-yl)-5,7- dihydrospiro[cyclopenta[b]pyridine-6,5′- [1,3]dioxane] | A |
| 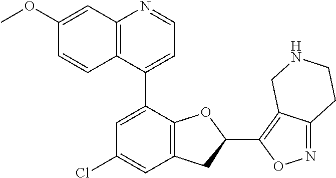 [SMILES: COc1ccc2c(-c3cc(Cl)cc4c3O[C@@H](c3onc5c3CNCC5)C4)ccnc2c1] | (R)-3-(5-chloro-7-(7-methoxyquinolin-4-yl)- 2,3-dihydrobenzofuran-2-yl)-4,5,6,7- tetrahydroisoxazolo[4,3-c]pyridine | A |
| 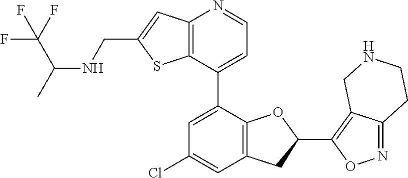 [SMILES: CC(NCc1cc2nccc(-c3cc(Cl)cc4c3O[C@@H](c3onc5c3CNCC5)C4)c2s1)C(F)(F)F] | N-((7-((R)-5-chloro-2-(4,5,6,7- tetrahydroisoxazolo[4,3-c]pyridin-3-yl)-2,3- dihydrobenzofuran-7-yl)thieno[3,2-b]pyridin-2- yl)methyl)-1,1,1-trifluoropropan-2-amine | A |
| 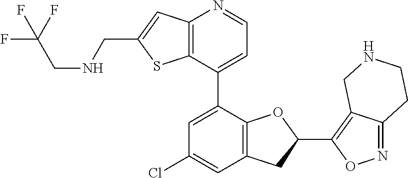 [SMILES: FC(F)(F)CNCc1cc2nccc(-c3cc(Cl)cc4c3O[C@@H](c3onc5c3CNCC5)C4)c2s1] | (R)-N-((7-(5-chloro-2-(4,5,6,7- tetrahydroisoxazolo[4,3-c]pyridin-3-yl)-2,3- dihydrobenzofuran-7-yl)thieno[3,2-b]pyridin-2- yl)methyl)-2,2,2-trifluoroethan-1-amine | A |
| 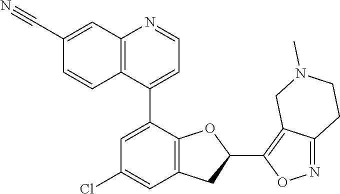 [SMILES: CN1CCc2noc([C@H]3Cc4cc(Cl)cc(-c5ccnc6cc(C#N)ccc56)c4O3)c2C1] | (R)-4-(5-chloro-2-(5-methyl-4,5,6,7- tetrahydroisoxazolo[4,3-c]pyridin-3-yl)-2,3- dihydrobenzofuran-7-yl)quinoline-7- carbonitrile | B |
| 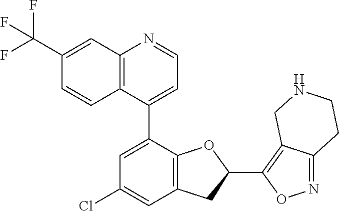 [SMILES: FC(F)(F)c1ccc2c(-c3cc(Cl)cc4c3O[C@@H](c3onc5c3CNCC5)C4)ccnc2c1] | (R)-3-(5-chloro-7-(7-(trifluoromethyl)quinolin- 4-yl)-2,3-dihydrobenzofuran-2-yl)-4,5,6,7- tetrahydroisoxazolo[4,3-c]pyridine | B |
| 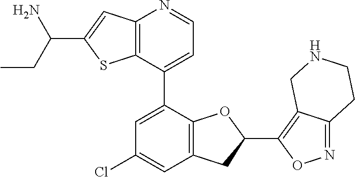 [SMILES: CCC(N)c1cc2nccc(-c3cc(Cl)cc4c3O[C@@H](c3onc5c3CNCC5)C4)c2s1] | 1-(7-((R)-5-chloro-2-(4,5,6,7- tetrahydroisoxazolo[4,3-c]pyridin-3-yl)-2,3- dihydrobenzofuran-7-yl)thieno[3,2-b]pyridin-2- yl)propan-1-amine | B |
| 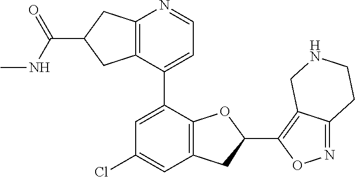 [SMILES: CNC(=O)C1Cc2nccc(-c3cc(Cl)cc4c3O[C@@H](c3onc5c3CNCC5)C4)c2C1] | 4-((R)-5-chloro-2-(4,5,6,7- tetrahydroisoxazolo[4,3-c]pyridin-3-yl)-2,3- dihydrobenzofuran-7-yl)-N-methyl-6,7- dihydro-5H-cyclopenta[b]pyridine-6- carboxamide | B |
| 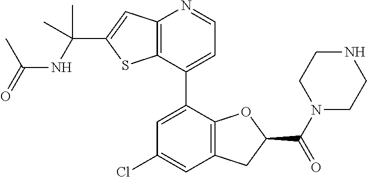 [SMILES: CC(=O)NC(C)(C)c1cc2nccc(-c3cc(Cl)cc4c3O[C@@H](C(=O)N3CCNCC3)C4)c2s1] | (R)-N-(2-(7-(5-chloro-2-(piperazine-1- carbonyl)-2,3-dihydrobenzofuran-7- yl)thieno[3,2-b]pyridin-2-yl)propan-2- yl)acetamide | B |
| 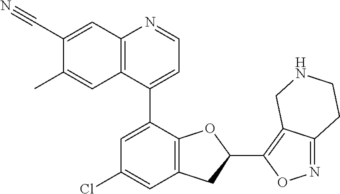 [SMILES: Cc1cc2c(-c3cc(Cl)cc4c3O[C@@H](c3onc5c3CNCC5)C4)ccnc2cc1C#N] | (R)-4-(5-chloro-2-(4,5,6,7- tetrahydroisoxazolo[4,3-c]pyridin-3-yl)-2,3- dihydrobenzofuran-7-yl)-6-methylquinoline-7- carbonitrile | B |
| 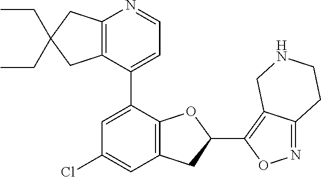 [SMILES: CCC1(CC)Cc2nccc(-c3cc(Cl)cc4c3O[C@@H](c3onc5c3CNCC5)C4)c2C1] | (R)-3-(5-chloro-7-(6,6-diethyl-6,7-dihydro-5H- cyclopenta[b]pyridin-4-yl)-2,3- dihydrobenzofuran-2-yl)-4,5,6,7- tetrahydroisoxazolo[4,3-c]pyridine | B |
| 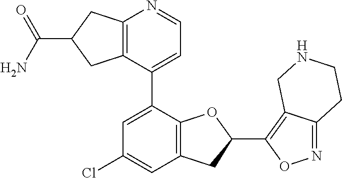 [SMILES: NC(=O)C1Cc2nccc(-c3cc(Cl)cc4c3O[C@@H](c3onc5c3CNCC5)C4)c2C1] | 4-((R)-5-chloro-2-(4,5,6,7- tetrahydroisoxazolo[4,3-c]pyridin-3-yl)-2,3- dihydrobenzofuran-7-yl)-6,7-dihydro-5H- cyclopenta[b]pyridine-6-carboxamide | B |
| 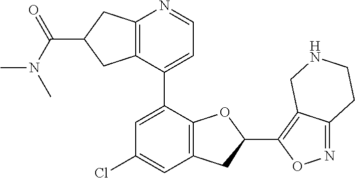 [SMILES: CN(C)C(=O)C1Cc2nccc(-c3cc(Cl)cc4c3O[C@@H](c3onc5c3CNCC5)C4)c2C1] | 4-((R)-5-chloro-2-(4,5,6,7- tetrahydroisoxazolo[4,3-c]pyridin-3-yl)-2,3- dihydrobenzofuran-7-yl)-N,N-dimethyl-6,7- dihydro-5H-cyclopenta[b]pyridine-6- carboxamide | B |
| 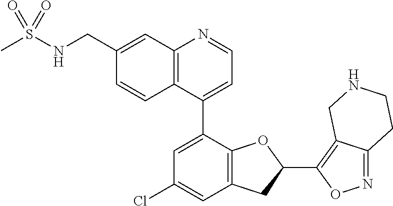 [SMILES: CS(=O)(=O)NCc1ccc2c(-c3cc(Cl)cc4c3O[C@@H](c3onc5c3CNCC5)C4)ccnc2c1] | (R)-N-((4-(5-chloro-2-(4,5,6,7- tetrahydroisoxazolo[4,3-c]pyridin-3-yl)-2,3- dihydrobenzofuran-7-yl)quinolin-7- yl)methyl)methanesulfonamide | B |
| 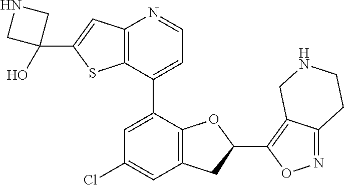 [SMILES: OC1(c2cc3nccc(-c4cc(Cl)cc5c4O[C@@H](c4onc6c4CNCC6)C5)c3s2)CNC1] | (R)-3-(7-(5-chloro-2-(4,5,6,7- tetrahydroisoxazolo[4,3-c]pyridin-3-yl)-2,3- dihydrobenzofuran-7-yl)thieno[3,2-b]pyridin-2- yl)azetidin-3-ol | B |
| 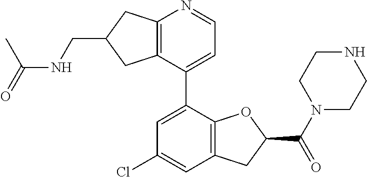 [SMILES: CC(=O)NCC1Cc2nccc(-c3cc(Cl)cc4c3O[C@@H](C(=O)N3CCNCC3)C4)c2C1] | N-((4-((R)-5-chloro-2-(piperazine-1-carbonyl)- 2,3-dihydrobenzofuran-7-yl)-6,7-dihydro-5H- cyclopenta[b]pyridin-6-yl)methyl)acetamide | B |
| 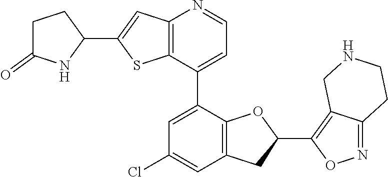 [SMILES: O=C1CCC(c2cc3nccc(-c4cc(Cl)cc5c4O[C@@H](c4onc6c4CNCC6)C5)c3s2)N1] | 5-(7-((R)-5-chloro-2-(4,5,6,7- tetrahydroisoxazolo[4,3-c]pyridin-3-yl)-2,3- dihydrobenzofuran-7-yl)thieno[3,2-b]pyridin-2- yl)pyrrolidin-2-one | B |
| 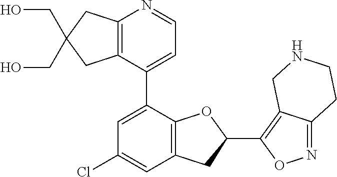 [SMILES: OCC1(CO)Cc2nccc(-c3cc(Cl)cc4c3O[C@@H](c3onc5c3CNCC5)C4)c2C1] | (R)-(4-(5-chloro-2-(4,5,6,7- tetrahydroisoxazolo[4,3-c]pyridin-3-yl)-2,3- dihydrobenzofuran-7-yl)-6,7-dihydro-5H- cyclopenta[b]pyridine-6,6-diyl)dimethanol | B |
| 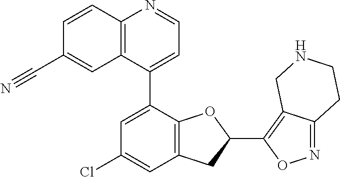 [SMILES: N#Cc1ccc2nccc(-c3cc(Cl)cc4c3O[C@@H](c3onc5c3CNCC5)C4)c2c1] | (R)-4-(5-chloro-2-(4,5,6,7- tetrahydroisoxazolo[4,3-c]pyridin-3-yl)-2,3- dihydrobenzofuran-7-yl)quinoline-6- carbonitrile | B |
| 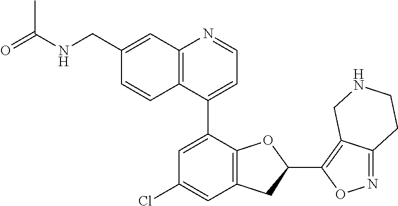 [SMILES: CC(=O)NCc1ccc2c(-c3cc(Cl)cc4c3O[C@@H](c3onc5c3CNCC5)C4)ccnc2c1] | (R)-N-((4-(5-chloro-2-(4,5,6,7- tetrahydroisoxazolo[4,3-c]pyridin-3-yl)-2,3- dihydrobenzofuran-7-yl)quinolin-7- yl)methyl)acetamide | B |
| 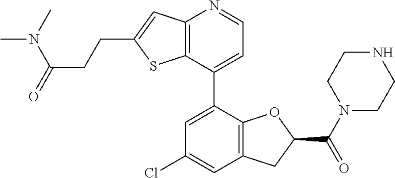 [SMILES: CN(C)C(=O)CCc1cc2nccc(-c3cc(Cl)cc4c3O[C@@H](C(=O)N3CCNCC3)C4)c2s1] | (R)-3-(7-(5-chloro-2-(piperazine-1-carbonyl)- 2,3-dihydrobenzofuran-7-yl)thieno[3,2- b]pyridin-2-yl)-N,N-dimethylpropanamide | B |
| 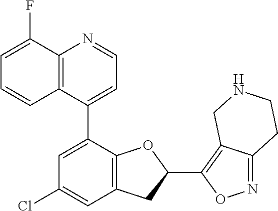 [SMILES: Fc1cccc2c(-c3cc(Cl)cc4c3O[C@@H](c3onc5c3CNCC5)C4)ccnc12] | (R)-3-(5-chloro-7-(8-fluoroquinolin-4-yl)-2,3- dihydrobenzofuran-2-yl)-4,5,6,7- tetrahydroisoxazolo[4,3-c]pyridine | B |
| 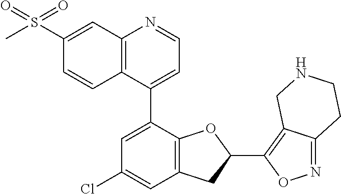 [SMILES: CS(=O)(=O)c1ccc2c(-c3cc(Cl)cc4c3O[C@@H](c3onc5c3CNCC5)C4)ccnc2c1] | (R)-3-(5-chloro-7-(7-(methylsulfonyl)quinolin- 4-yl)-2,3-dihydrobenzofuran-2-yl)-4,5,6,7- tetrahydroisoxazolo[4,3-c]pyridine | B |
| 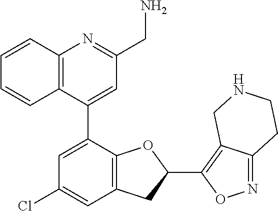 [SMILES: NCc1cc(-c2cc(Cl)cc3c2O[C@@H](c2onc4c2CNCC4)C3)c2ccccc2n1] | (R)-(4-(5-chloro-2-(4,5,6,7- tetrahydroisoxazolo[4,3-c]pyridin-3-yl)-2,3- dihydrobenzofuran-7-yl)quinolin-2- yl)methanamine | B |
| 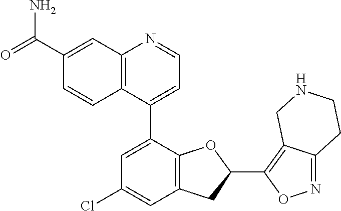 [SMILES: NC(=O)c1ccc2c(-c3cc(Cl)cc4c3O[C@@H](c3onc5c3CNCC5)C4)ccnc2c1] | (R)-4-(5-chloro-2-(4,5,6,7- tetrahydroisoxazolo[4,3-c]pyridin-3-yl)-2,3- dihydrobenzofuran-7-yl)quinoline-7- carboxamide | B |
| 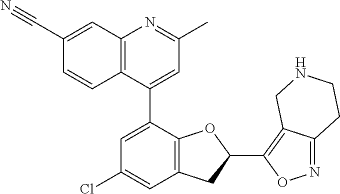 [SMILES: Cc1cc(-c2cc(Cl)cc3c2O[C@@H](c2onc4c2CNCC4)C3)c2ccc(C#N)cc2n1] | (R)-4-(5-chloro-2-(4,5,6,7- tetrahydroisoxazolo[4,3-c]pyridin-3-yl)-2,3- dihydrobenzofuran-7-yl)-2-methylquinoline-7- carbonitrile | C |
| 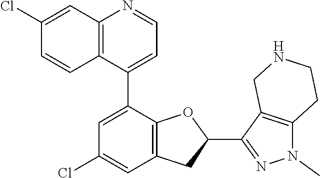 [SMILES: Cn1nc([C@H]2Cc3cc(Cl)cc(-c4ccnc5cc(Cl)ccc45)c3O2)c2c1CCNC2] | (R)-7-chloro-4-(5-chloro-2-(1-methyl-4,5,6,7- tetrahydro-1H-pyrazolo[4,3-c]pyridin-3-yl)- 2,3-dihydrobenzofuran-7-yl)quinoline | C |
| 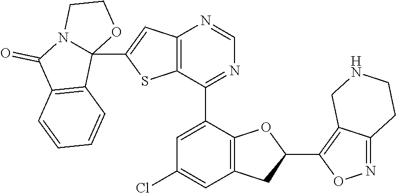 [SMILES: O=C1c2ccccc2C2(c3cc4ncnc(-c5cc(Cl)cc6c5O[C@@H](c5onc7c5CNCC7)C6)c4s3)OCCN12] | 9b-(4-((R)-5-chloro-2-(4,5,6,7- tetrahydroisoxazolo[4,3-c]pyridin-3-yl)-2,3- dihydrobenzofuran-7-yl)thieno[3,2- d]pyrimidin-6-yl)-2,3-dihydrooxazolo[2,3- a]isoindol-5(9bH)-one | C |
| 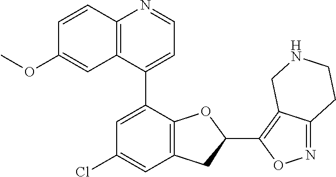 [SMILES: COc1ccc2nccc(-c3cc(Cl)cc4c3O[C@@H](c3onc5c3CNCC5)C4)c2c1] | (R)-3-(5-chloro-7-(6-methoxyquinolin-4-yl)- 2,3-dihydrobenzofuran-2-yl)-4,5,6,7- tetrahydroisoxazolo[4,3-c]pyridine | C |
| 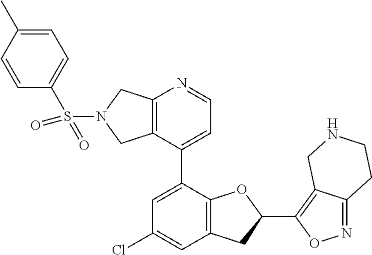 [SMILES: Cc1ccc(S(=O)(=O)N2Cc3nccc(-c4cc(Cl)cc5c4O[C@@H](c4onc6c4CNCC6)C5)c3C2)cc1] | (R)-3-(5-chloro-7-(6-tosyl-6,7-dihydro-5H- pyrrolo[3,4-b]pyridin-4-yl)-2,3- dihydrobenzofuran-2-yl)-4,5,6,7- tetrahydroisoxazolo[4,3-c]pyridine | C |
| 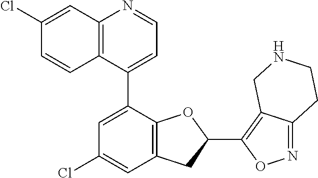 [SMILES: Clc1cc2c(c(-c3ccnc4cc(Cl)ccc34)c1)O[C@@H](c1onc3c1CNCC3)C2] | (R)-3-(5-chloro-7-(7-chloroquinolin-4-yl)-2,3- dihydrobenzofuran-2-yl)-4,5,6,7- tetrahydroisoxazolo[4,3-c]pyridine | A |
| 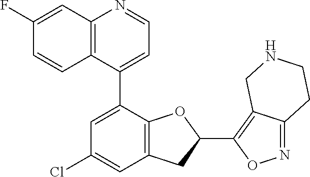 [SMILES: Fc1ccc2c(-c3cc(Cl)cc4c3O[C@@H](c3onc5c3CNCC5)C4)ccnc2c1] | (R)-3-(5-chloro-7-(7-fluoroquinolin-4-yl)-2,3- dihydrobenzofuran-2-yl)-4,5,6,7- tetrahydroisoxazolo[4,3-c]pyridine | A |
| 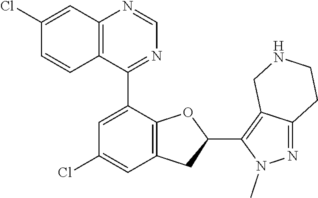 [SMILES: Cn1nc2c(c1[C@H]1Cc3cc(Cl)cc(-c4ncnc5cc(Cl)ccc45)c3O1)CNCC2] | (R)-7-chloro-4-(5-chloro-2-(2-methyl-4,5,6,7- tetrahydro-2H-pyrazolo[4,3-c]pyridin-3-yl)- 2,3-dihydrobenzofuran-7-yl)quinazoline | A |
| 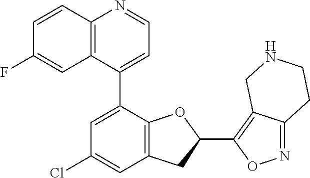 [SMILES: Fc1ccc2nccc(-c3cc(Cl)cc4c3O[C@@H](c3onc5c3CNCC5)C4)c2c1] | (R)-3-(5-chloro-7-(6-fluoroquinolin-4-yl)-2,3- dihydrobenzofuran-2-yl)-4,5,6,7- tetrahydroisoxazolo[4,3-c]pyridine | A |
| 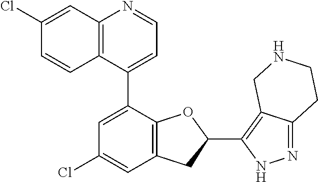 [SMILES: Clc1cc2c(c(-c3ccnc4cc(Cl)ccc34)c1)O[C@@H](c1[nH]nc3c1CNCC3)C2] | (R)-7-chloro-4-(5-chloro-2-(4,5,6,7-tetrahydro- 2H-pyrazolo[4,3-c]pyridin-3-yl)-2,3- dihydrobenzofuran-7-yl)quinoline | B |
| 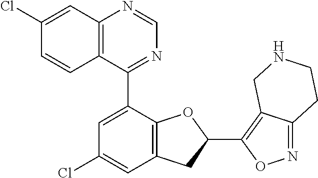 [SMILES: Clc1cc2c(c(-c3ncnc4cc(Cl)ccc34)c1)O[C@@H](c1onc3c1CNCC3)C2] | (R)-3-(5-chloro-7-(7-chloroquinazolin-4-yl)- 2,3-dihydrobenzofuran-2-yl)-4,5,6,7- tetrahydroisoxazolo[4,3-c]pyridine | B |
| 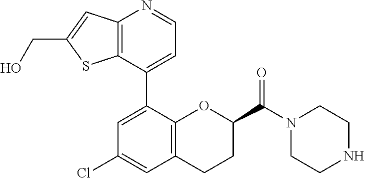 [SMILES: O=C([C@H]1CCc2cc(Cl)cc(-c3ccnc4cc(CO)sc34)c2O1)N1CCNCC1] | (R)-(6-chloro-8-(2-(hydroxymethyl)thieno[3,2- b]pyridin-7-yl)chroman-2-yl)(piperazin-1- yl)methanone | A |
| 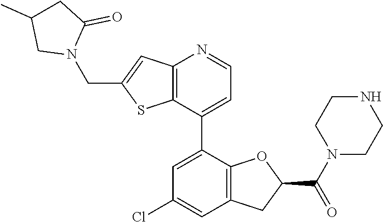 [SMILES: CC1CC(=O)N(Cc2cc3nccc(-c4cc(Cl)cc5c4O[C@@H](C(=O)N4CCNCC4)C5)c3s2)C1] | 1-((7-((R)-5-chloro-2-(piperazine-1-carbonyl)- 2,3-dihydrobenzofuran-7-yl)thieno[3,2- b]pyridin-2-yl)methyl)-4-methylpyrrolidin-2- one | A |
| 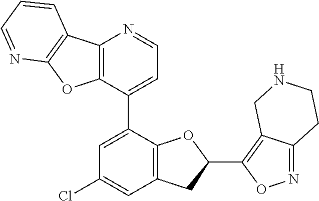 [SMILES: Clc1cc2c(c(-c3ccnc4c3oc3ncccc34)c1)O[C@@H](c1onc3c1CNCC3)C2] | (R)-4-(5-chloro-2-(4,5,6,7- tetrahydroisoxazolo[4,3-c]pyridin-3-yl)-2,3- dihydrobenzofuran-7-yl)furo[2,3-b:4,5- b′]dipyridine | |
| 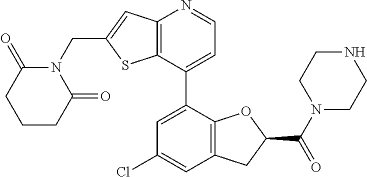 [SMILES: O=C([C@H]1Cc2cc(Cl)cc(-c3ccnc4cc(CN5C(=O)CCCC5=O)sc34)c2O1)N1CCNCC1] | (R)-1-((7-(5-chloro-2-(piperazine-1-carbonyl)- 2,3-dihydrobenzofuran-7-yl)thieno[3,2- b]pyridin-2-yl)methyl)piperidine-2,6-dione | A |
| 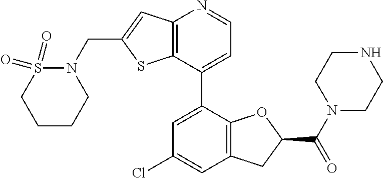 [SMILES: O=C([C@H]1Cc2cc(Cl)cc(-c3ccnc4cc(CN5CCCCS5(=O)=O)sc34)c2O1)N1CCNCC1] | (R)-(5-chloro-7-(2-((1,1-dioxido-1,2-thiazinan- 2-yl)methyl)thieno[3,2-b]pyridin-7-yl)-2,3- dihydrobenzofuran-2-yl)(piperazin-1- yl)methanone | A |
| 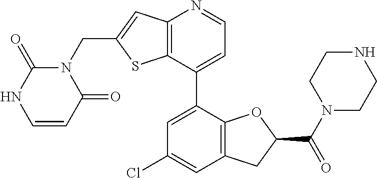 [SMILES: O=C([C@H]1Cc2cc(Cl)cc(-c3ccnc4cc(Cn5c(=O)cc[nH]c5=O)sc34)c2O1)N1CCNCC1] | (R)-3-((7-(5-chloro-2-(piperazine-1-carbonyl)- 2,3-dihydrobenzofuran-7-yl)thieno[3,2- b]pyridin-2-yl)methyl)pyrimidine-2,4(1H,3H)- dione | A |
| 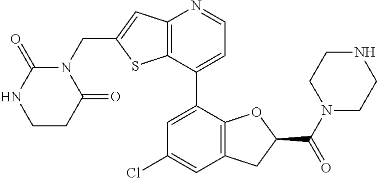 [SMILES: O=C([C@H]1Cc2cc(Cl)cc(-c3ccnc4cc(CN5C(=O)CCNC5=O)sc34)c2O1)N1CCNCC1] | (R)-3-((7-(5-chloro-2-(piperazine-1-carbonyl)- 2,3-dihydrobenzofuran-7-yl)thieno[3,2- b]pyridin-2-yl)methyl)dihydropyrimidine- 2,4(1H,3H)-dione | A |
| 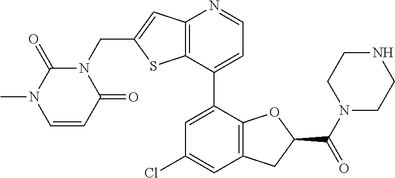 [SMILES: Cn1ccc(=O)n(Cc2cc3nccc(-c4cc(Cl)cc5c4O[C@@H](C(=O)N4CCNCC4)C5)c3s2)c1=O] | (R)-3-((7-(5-chloro-2-(piperazine-1-carbonyl)- 2,3-dihydrobenzofuran-7-yl)thieno[3,2- b]pyridin-2-yl)methyl)-1-methylpyrimidine- 2,4(1H,3H)-dione | A |
| 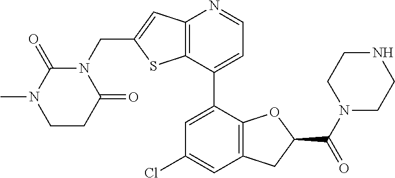 [SMILES: CN1CCC(=O)N(Cc2cc3nccc(-c4cc(Cl)cc5c4O[C@@H](C(=O)N4CCNCC4)C5)c3s2)C1=O] | (R)-3-((7-(5-chloro-2-(piperazine-1-carbonyl)- 2,3-dihydrobenzofuran-7-yl)thieno[3,2- b]pyridin-2-yl)methyl)-1- methyldihydropyrimidine-2,4(1H,3H)-dione | A |

1. A compound of structural Formula (VII) or a pharmaceutically acceptable salt thereof: 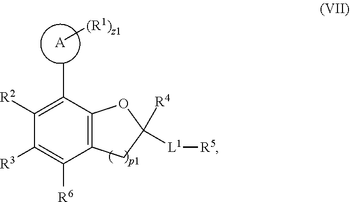
wherein:
Ring A-(R¹)_{z1} is 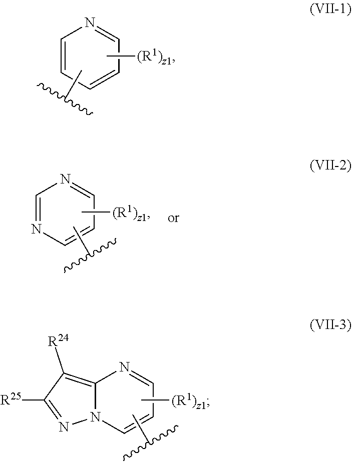
wherein,
p1 is 1 or 2;
L¹ is a bond, —O—, —S—, —NH—, —C(O)—, —C(O)NH, —C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is independently halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —N(O)ₘ₁, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein two R¹ substituents bonded to adjacent carbon atoms may optionally be joined to form a substituted or unsubstituted cycloalkyl, substituted or unsubstituted heterocycloalkyl, substituted or unsubstituted aryl, or substituted or unsubstituted heteroaryl;
R² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₂H, —SOᵥ₂NH₂, —NHNH₂, —ONH₂, —NHC(O)NHNH₂, —NHC(O)NH₂, —N(O)ₘ₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₃H, —SOᵥ₃NH₂, —NHNH₂, —ONH₂, —NHC(O)NHNH₂, —NHC(O)NH₂, —N(O)ₘ₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₄H, —SOᵥ₄NH₂, —NHNH₂, —ONH₂, —NHC(O)NHNH₂, —NHC(O)NH₂, —N(O)ₘ₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₅R^{5A}, —SOᵥ₅NR^{5B}R^{5C}, —NHNR^{5B}R^{5C}, —ONR^{5B}R^{5C}, —NHC(O)NHNR^{5B}R^{5C}, —NHC(O)NR^{5B}R^{5C}, —N(O)ₘ₅, —NR^{5B}R^{5C}, —C(O)R^{5D}, —C(O)OR^{5D}, —C(O)NR^{5B}R^{5C}, —OR^{5A}, —NR^{5B}SO₂R^{5A}, —NR^{5B}C(O)R^{5D}, —NR^{5B}C(O)OR^{5D}, —NR^{5B}OR^{5D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₆H, —SOᵥ₆NH₂, —NHNH₂, —ONH₂, —NHC(O)NHNH₂, —NHC(O)NH₂, —N(O)ₘ₆, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₄H, —SOᵥ₂₄NH₂, —NHNH₂, —ONH₂, —NHC(O)NHNH₂, —NHC(O)NH₂, —N(O)ₘ₂₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁵ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₅R^{25A}, —SOᵥ₂₅NR^{25B}R^{25C}, —NHNR^{25B}R^{25C}, —ONR^{25B}R^{25C}, —NHC(O)NHNR^{25B}R^{25C}, —NHC(O)NR^{25B}R^{25C}, —N(O)ₘ₂₅, —NR^{25B}R^{25C}, —C(O)R^{25D}, —C(O)OR^{25D}, —C(O)NR^{25B}R^{25C}, —OR^{25A}, —NR^{25B}SO₂R^{25A}, —NR^{25B}C(O)R^{25D}, —NR^{25B}C(O)OR^{25D}, —NR^{25B}OR^{25D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R^{1A}, R^{1B}, R^{1C}, R^{1D}, R^{5A}, R^{5B}, R^{5C}, R^{5D}, R^{25A}, R^{25B}, R^{25C}, and R^{25D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C}, R^{5B} and R^{5C}, or R^{25B} and R^{25C} substituents bonded to the same nitrogen atom may optionally be joined to form a substituted or unsubstituted heterocycloalkyl or substituted or unsubstituted heteroaryl;
n1, n2, n3, n4, n5, n6, n24, and n25 are independently an integer from 0 to 4;
m1, m2, m3, m4, m5, m6, m24, m25, v1, v2, v3, v4, v5, v6, v24, and v25 are independently 1 or 2; and
z1 is an integer from 0 to 4.

2. (canceled)

3. The compound of claim 1, or a pharmaceutically acceptable salt thereof, wherein the compound has structural Formula (IX): 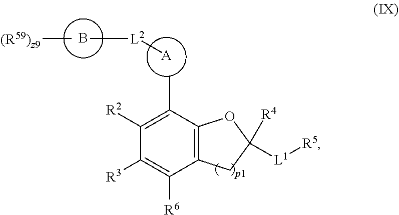
wherein:
Ring B is substituted or unsubstituted cycloalkyl, substituted or unsubstituted heterocycloalkyl, substituted or unsubstituted aryl, or substituted or unsubstituted heteroaryl;
L² is a bond, —O—, —S—, —NH—, —C(O)—, —C(O)NH, —C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R⁵⁹ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₅₉H, —SOᵥ₅₉NH₂, —NHNH₂, —ONH₂, —NHC(O)NHNH₂, —NHC(O)NH₂, —N(O)ₘ₅₉, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein two R⁵⁹ substituents bonded to adjacent atoms may optionally be joined to form a substituted or unsubstituted cycloalkyl, substituted or unsubstituted heterocycloalkyl, substituted or unsubstituted aryl, or substituted or unsubstituted heteroaryl;
n59 is an integer from 0 to 4;
m59 and v59 are independently 1 or 2; and
z9 is an integer from 0 to 6.

4.-7. (canceled)

8. The compound of claim 1, or a pharmaceutically acceptable salt thereof, wherein the compound has structural Formula (X): 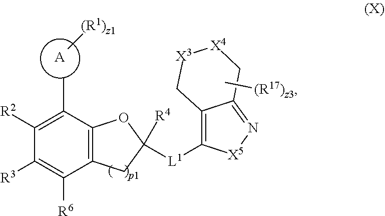
wherein:
X³ is 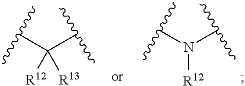
X⁴ is 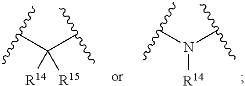
X⁵ is —O— or 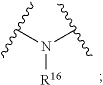
R¹² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —N₃, —CN, —SO₂CH₃, —SOₙ₁₂H, —SOᵥ₁₂NH₂, —NHNH₂, —ONH₂, —NHC(O)NHNH₂, —NHC(O)NH₂, —N(O)ₘ₁₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹³ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₃H, —SOᵥ₁₃NH₂, —NHNH₂, —ONH₂, —NHC(O)NHNH₂, —NHC(O)NH₂, —N(O)ₘ₁₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² and R¹³ are optionally together oxo;
R¹⁴ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₄H, —SOᵥ₁₄NH₂, —NHNH₂, —ONH₂, —NHC(O)NHNH₂, —NHC(O)NH₂, —N(O)ₘ₁₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁵ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₅H, —SOᵥ₁₅NH₂, —NHNH₂, —ONH₂, —NHC(O)NHNH₂, —NHC(O)NH₂, —N(O)ₘ₁₅, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁴ and R¹⁵ are optionally together oxo;
R¹⁶ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₆H, —SOᵥ₁₆NH₂, —NHNH₂, —ONH₂, —NHC(O)NHNH₂, —NHC(O)NH₂, —N(O)ₘ₁₆, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R¹⁷ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₁₇H, —SOᵥ₁₇NH₂, —NHNH₂, —ONH₂, —NHC(O)NHNH₂, —NHC(O)NH₂, —N(O)ₘ₁₇, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n12, n13, n14, n15, n16 and n17 are independently an integer from 0 to 4;
m12, m13, m14, m15, m16, m17, v12, v13, v14, v15, v16 and v17 are independently 1 or 2; and
z3 is an integer from 0 to 4.

9.-17. (canceled)

18. The compound of claim 1, or a pharmaceutically acceptable salt thereof, wherein the compound has structural Formula (XI):
3. 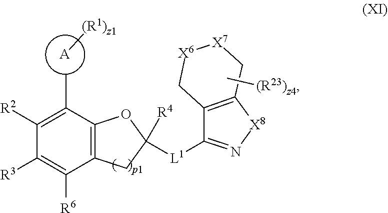
wherein:
X⁶ is 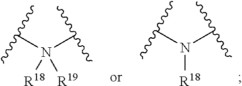
X⁷ is 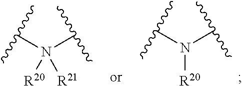
X⁸ is —O— or 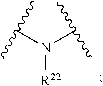
R¹⁸ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₈H, —SOᵥ₁₈NH₂, —NHNH₂, —ONH₂, —NHC(O)NHNH₂, —NHC(O)NH₂, —N(O)ₘ₁₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₁₉H, —SOᵥ₁₉NH₂, —NHNH₂, —ONH₂, —NHC(O)NHNH₂, —NHC(O)NH₂, —N(O)ₘ₁₉, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁸ and R¹⁹ are optionally together oxo;
R²⁰ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₂₀H, —SOᵥ₂₀NH₂, —NHNH₂, —ONH₂, —NHC(O)NHNH₂, —NHC(O)NH₂, —N(O)ₘ₂₀, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²¹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SO₂CH₃, —SOₙ₂₁H, —SOᵥ₂₁NH₂, —NHNH₂, —ONH₂, —NHC(O)NHNH₂, —NHC(O)NH₂, —N(O)ₘ₂₁, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁰ and R²¹ are optionally together oxo;
R²² is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₂H, —SO₂CH₃, —SOᵥ₂₂NH₂, —NHNH₂, —ONH₂, —NHC(O)NHNH₂, —NHC(O)NH₂, —N(O)ₘ₂₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R²³ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₂₃H, —SOᵥ₂₃NH₂, —NHNH₂, —ONH₂, —NHC(O)NHNH₂, —NHC(O)NH₂, —N(O)ₘ₂₃, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n18, n19, n20, n21, n22 and n23 are independently an integer from 0 to 4;
m18, m19, m20, m21, m22, m23, v18, v19, v20, v21, v22 and v23 are independently 1 or 2; and
z4 is an integer from 0 to 4.

19.-27. (canceled)

28. The compound of claim 1, or a pharmaceutically acceptable salt thereof, wherein the compound has structural Formula (X-1A):

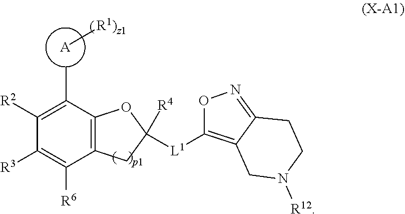

29. The compound of claim 1, or a pharmaceutically acceptable salt thereof, wherein the compound has structural Formula (XII):
4. 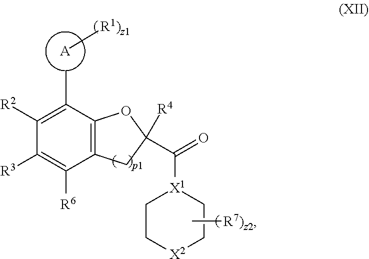
wherein:
X¹ is or 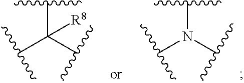
X² is 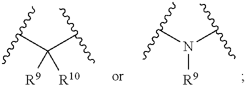
n7, n8, n9 and n10 are independently an integer from 0 to 4;
m7, m8, m9, m10, v7, v8, v9 and v10 are independently 1 or 2;
z2 is an integer from 0 to 8;
R⁷ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₇H, —SOᵥ₇NH₂, —NHNH₂, —ONH₂, —NHC(O)NHNH₂, —NHC(O)NH₂, —N(O)ₘ₇, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₈H, —SOᵥ₈NH₂, —NHNH₂, —ONH₂, —NHC(O)NHNH₂, —NHC(O)NH₂, —N(O)ₘ₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₉R^{9A}, —SOᵥ₉NR^{9B}R^{9C}, —NHNR^{9B}R^{9C}, —ONR^{9B}R^{9C}, —NHC(O)NHNR^{9B}R^{9C}, —NHC(O)NR^{9B}R^{9C}, —N(O)ₘ₉, —NR^{9B}R^{9C}, —C(O)R^{9D}, —C(O)OR^{9D}, —C(O)NR^{9B}R^{9C}, —OR^{9A}, —NR^{9B}SO₂R^{9A}, —NR^{9B}C(O)R^{9D}, —NR^{9B}C(O)OR^{9D}, —NR^{9B}OR^{9D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ is hydrogen, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —SOₙ₁₀R^{10A}, —SOᵥ₁₀NR^{10B}R^{10C}, —NHNR^{10B}R^{10C}, —ONR^{10B}R^{10C}, —NHC(O)NHNR^{10B}R^{10C}, —NHC(O)NR^{10B}R^{10C}, —N(O)ₘ₁₀, —NR^{10B}R^{10C}, —C(O)R^{10D}, —C(O)OR^{10D}, —C(O)NR^{10B}R^{10C}, —OR^{10A}, —NR^{10B}SO₂R^{10A}, —NR^{10B}C(O)R^{10D}, —NR^{10B}C(O)OR^{10D}, —NR^{10B}OR^{10D}, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ and R¹⁰ are optionally together oxo; and
R^{9A}, R^{9B}, R^{9C}, R^{9D}, R^{10A}, R^{10B}, R^{10C} and R^{10D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{9B} and R^{9C} or R^{10B} and R^{10C} substituents bonded to the same nitrogen atom may optionally be joined to form a substituted or unsubstituted heterocycloalkyl or substituted or unsubstituted heteroaryl.

30.-53. (canceled)

54. The compound of claim 1, wherein the compound is of structural Formula (I): 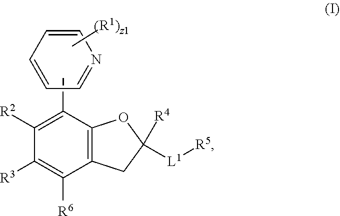
or a pharmaceutically acceptable salt thereof.

55. The compound of claim 1, wherein the compound is of structural Formula (II): 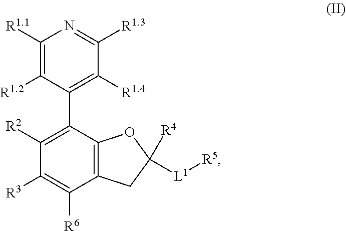
or a pharmaceutically acceptable salt thereof,
wherein:
n1.1, n1.2, n1.3 and n1.4 are independently and integer from 0 to 4; m1.1, m1.2, m1.3, m1.4, v1.1, v1.2, v1.3 and v1.4 are independently 1 or 2;
R^{1.1} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.1}R^{1.1A}, —SO_{v1.1}NR^{1.1B}R^{1.1C}, —NHNR^{1.1B}R^{1.1C}, —ONR^{1.1B}R^{1.1C}, —NHC(O)NHNR^{1.1B}R^{1.1C}, —NHC(O)NR^{1.1B}R^{1.1C}, —N(O)_{m1.1}, —NR^{1.1B}R^{1.1C}, —C(O)R^{1.1D}, —C(O)OR^{1.1D}, —C(O)NR^{1.1B}R^{1.1C}, —OR^{1.1A}, —NR^{1.1B}SO₂R^{1.1A}, —NR^{1.1B}C(O)R^{1.1D}, —NR^{1.1B}C(O)OR^{1.1D}, —NR^{1.1B}OR^{1.1D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R^{1.2} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.2}H, —SO_{v1.2}NH₂, —NHNH₂, —ONH₂, —NHC(O)NHNH₂, —NHC(O)NH₂, —N(O)_{m1.2}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1.3} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.3}H, —SO_{v1.3}NH₂, —NHNH₂, —ONH₂, —NHC(O)NHNH₂, —NHC(O)NH₂, —N(O)_{m1.3}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1.4} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.4}H, —SO_{v1.4}NH₂, —NHNH₂, —ONH₂, —NHC(O)NHNH₂, —NHC(O)NH₂, —N(O)_{m1.4}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{1.1A}, R^{1.1B}, R^{1.1C} and R^{1.1D} are independently hydrogen, halogen, —CF₃, —CCl₃, —CBr₃, —Cl₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1.1B} and R^{1.1C} substituents bonded to the same nitrogen atom may optionally be joined to form a substituted or unsubstituted heterocycloalkyl or substituted or unsubstituted heteroaryl; and wherein R^{1.1} and R^{1.2} or R^{1.3} and R^{1.4} may optionally be joined to form a substituted or unsubstituted cycloalkyl, substituted or unsubstituted heterocycloalkyl, substituted or unsubstituted aryl or substituted or unsubstituted heteroaryl, and further wherein at least one of R^{1.1}, R^{1.2}, R^{1.3} and R^{1.4} is not hydrogen.

56. The compound of claim 1, wherein the compound is of structural Formula (III): 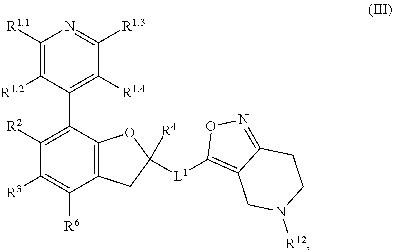
or a pharmaceutically acceptable salt thereof:
wherein:
n1.1, n1.2, n1.3, n1.4 and n12 are independently and integer from 0 to 4;
m1.1, m1.2, m1.3, m1.4, m12, v1.1, v1.2, v1.3, v1.4 and v12 are independently 1 or 2;
R^{1.1} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.1}R^{1.11A}, —SO_{v1.1}NR^{1.1B}R^{1.1C}, —NHNR^{1.1B}R^{1.1C}, —ONR^{1.1B}R^{1.1C}, —NHC(O)NHNR^{1.1B}R^{1.1C}, —NHC(O)NR^{1.1B}R^{1.1C}, —N(O)_{m1.1}, —NR^{1.1B}R^{1.1C}, —C(O)R^{1.1D}, —C(O)OR^{1.1D}, —C(O)NR^{1.1B}R^{1.1C}, —OR^{1.1A}, —NR^{1.1B}SO₂R^{1.1A}, —NR^{1.1B}C(O)R^{1.1D}, —NR^{1.1B}C(O)OR^{1.1D}, —NR^{1.1B}OR^{1.1D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R^{1.2} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.2}H, —SO_{v1.2}NH₂, —NHNH₂, —ONH₂, —NHC(O)NHNH₂, —NHC(O)NH₂, —N(O)_{m1.2}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1.3} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.3}H, —SO_{v1.3}NH₂, —NHNH₂, —ONH₂, —NHC(O)NHNH₂, —NHC(O)NH₂, —N(O)_{m1.3}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1.4} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.4}H, —SO_{v1.4}NH₂, —NHNH₂, —ONH₂, —NHC(O)NHNH₂, —NHC(O)NH₂, —N(O)_{m1.4}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₂H, —SOᵥ₁₂NH₂, —NHNH₂, —ONH₂, —NHC(O)NHNH₂, —NHC(O)NH₂, —N(O)ₘ₁₂, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R^{1.1A}, R^{1.1B}, R^{1.1C} and R^{1.1D} are independently hydrogen, halogen, —CF₃, —CCl₃, —CBr₃, —Cl₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1.1B} and R^{1.1C} substituents bonded to the same nitrogen atom may optionally be joined to form a substituted or unsubstituted heterocycloalkyl or substituted or unsubstituted heteroaryl; and
wherein R^{1.1} and R^{1.2} or R^{1.3} and R^{1.4} may optionally be joined to form a substituted or unsubstituted cycloalkyl, substituted or unsubstituted heterocycloalkyl, substituted or unsubstituted aryl or substituted or unsubstituted heteroaryl, and further wherein at least one of R^{1.1}, R^{1.2}, R^{1.3} and R^{1.4} is not hydrogen.

57. The compound of claim 1, wherein the compound is of structural Formula (IV): 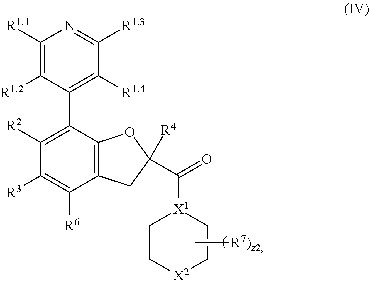
or a pharmaceutically acceptable salt thereof,
wherein:
X¹ is C(R⁸) or N;
X² is C(R⁹)(R¹⁰) or N(R⁹);
n1.1, n1.2, n1.3, n1.4, n7, n8, n9 and n10 are independently and integer from 0 to 4;
m1, m1.2, m1.3, m1.4, m7, m8, m9, m10, v1.1, v1.2, v1.3, v1.4, v7, v8, v9 and v10 are independently 1 or 2;
z2 is an integer from 0 to 8;
R^{1.1} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.1}R^{1.1A}, —SO_{v1.1}NR^{1.1B}R^{1.1C}, —NHNR^{1.1B}R^{1.1C}, —ONR^{1.1B}R^{1.1C}, —NHC(O)NHNR^{1.1B}R^{1.1C}, —NHC(O)NR^{1.1B}R^{1.1C}, —N(O)_{m1.1}, —NR^{1.1B}R^{1.1C}, —C(O)R^{1.1D}, —C(O)OR^{1.1D}, —C(O)NR^{1.1B}R^{1.1C}, —OR^{1.1A}, —NR^{1.1B}SO₂R^{1.1A}, —NR^{1.1B}C(O)R^{1.1D}, —NR^{1.1B}C(O)OR^{1.1D}, —NR^{1.1B}OR^{1.1D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R^{1.2} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.2}H, —SO_{v1.2}NH₂, —NHNH₂, —ONH₂, —NHC(O)NHNH₂, —NHC(O)NH₂, —N(O)_{m1.2}, —NH₂, —C(O)H, —C(O)OH, —C(O)NH₂, —OH, —NHSO₂H, —NHC(O)H, —NHC(O)OH, —NHOH—OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1.3} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.3}H, —SO_{v1.3}NH₂, —NHNH₂, —ONH₂, —NHC(O)NHNH₂, —NHC(O)NH₂, —N(O)_{m1.3}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1.4} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.4}H, —SO_{v1.4}NH₂, —NHNH₂, —ONH₂, —NHC(O)NHNH₂, —NHC(O)NH₂, —N(O)_{m1.4}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₇H, —SOᵥ₇NH₂, —NHNH₂, —ONH₂, —NHC(O)NHNH₂, —NHC(O)NH₂, —N(O)ₘ₇, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R⁸ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₈H, —SOᵥ₈NH₂, —NHNH₂, —ONH₂, —NHC(O)NHNH₂, —NHC(O)NH₂, —N(O)ₘ₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R⁹ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₉R^{9A}, —SOᵥ₉NR^{9B}R^{9C}, —NHNR^{9B}R^{9C}, —ONR^{9B}R^{9C}, —NHC(O)NHNR^{9B}R^{9C}, —NHC(O)NR^{9B}R^{9C}, —N(O)ₘ₉, —NR^{9B}R^{9C}, —C(O)R^{9D}, —C(O)OR^{9D}, —C(O)NR^{9B}R^{9C}, —OR^{9A}, —NR^{9B}SO₂R^{9A}, —NR^{9B}C(O)R^{9D}, —NR^{9B}C(O)OR^{9D}, —NR^{9B}OR^{9D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R¹⁰ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₁₀R^{10A}, —SOᵥ₁₀NR^{10B}R^{10C}, —NHNR^{10B}R^{10C}, —ONR^{10B}R^{10C}, —NHC(O)NHNR^{10B}R^{10C}, —NHC(O)NR^{10B}R^{10C}, —N(O)ₘ₁₀, —NR^{10B}R^{10C}, —C(O)R^{10D}, —C(O)OR^{10D}, —C(O)NR^{10B}R^{10C}, —OR^{10A}, —NR^{10B}SO₂R^{10A}, —NR^{10B}C(O)R^{10D}, —NR^{10B}C(O)OR^{10D}, —NR^{10B}OR^{10D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and
R^{1.1A}, R^{1.1B}, R^{1.1C}, R^{1.1D}, R^{9A}, R^{9B}, R^{9C}, R^{9A}, R^{10A}, R^{10B}, R^{10C} and R^{10D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1.1B} and R^{1.1C}, R^{9B} and R^{9C} or R^{10B} and R^{10C} substituents bonded to the same nitrogen atom may optionally be joined to form a substituted or unsubstituted heterocycloalkyl or substituted or unsubstituted heteroaryl; and wherein R^{1.1} and R^{1.2} or R^{1.3} and R^{1.4} may optionally be joined to form a substituted or unsubstituted cycloalkyl, substituted or unsubstituted heterocycloalkyl, substituted or unsubstituted aryl or substituted or unsubstituted heteroaryl, and further wherein at least one of R^{1.1}, R^{1.2}, R^{1.3} and R^{1.4} is not hydrogen.

58. The compound of claim 1, wherein the compound is of structural Formula (V): 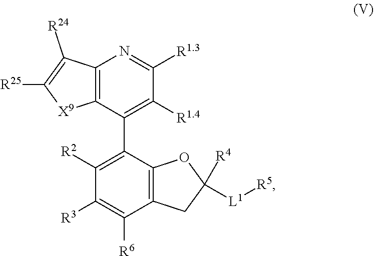
or a pharmaceutically acceptable salt thereof,
wherein:
n1.3, n1.4, n24 and n25 are independently and integer from 0 to 4;
m1.3, m1.4, m24, m25, v1.3, v1.4, v24 and v25 are independently 1 or 2;
X⁹ is —O— or —S—;
R¹³ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.3}H, —SO_{v1.3}NH₂, —NHNH₂, —ONH₂, —NHC(O)NHNH₂, —NHC(O)NH₂, —N(O)_{m1.3}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1.4} is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.4}H, —SO_{v1.4}NH₂, —NHNH₂, —ONH₂, —NHC(O)NHNH₂, —NHC(O)NH₂, —N(O)_{m1.4}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁴ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₄H, —SOᵥ₂₄NH₂, —NHNH₂, —ONH₂, —NHC(O)NHNH₂, —NHC(O)NH₂, —N(O)ₘ₂₄, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁵ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₅R^{25A}, —SOᵥ₂₅NR^{25B}R^{25C}, —NHNR^{25B}R^{25C}, —ONR^{25B}R^{25C}, —NHC(O)NHNR^{25B}R^{25C}, —NHC(O)NR^{25B}R^{25C}, —N(O)ₘ₂₅, —NR^{25B}R^{25C}, —C(O)R^{25D}, —C(O)OR^{25D}, —C(O)NR^{25B}R^{25C}, —OR^{25A}, —NR^{25B}SO₂R^{25A}, —NR^{25B}C(O)R^{25D}, —NR^{25B}C(O)OR^{25D}, —NR^{25B}OR^{25D}, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; and
R^{25A}, R^{25B}, R^{25C} and R^{25D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{25B} and R^{25C} substituents bonded to the same nitrogen atom may optionally be joined to form a substituted or unsubstituted heterocycloalkyl or substituted or unsubstituted heteroaryl.

59. The compound of claim 1, wherein the compound is of structural Formula (VI): 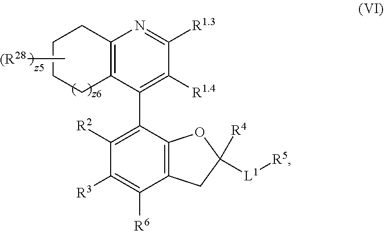
or a pharmaceutically acceptable salt thereof,
wherein:
z5 is an integer from 0 to 8;
z6 is 0 or 1;
n1.3, n1.4 and n28 are independently and integer from 0 to 4;
m1.3, m1.4, m28, v1.3, v1.4 and v28 are independently 1 or 2;
R¹³ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.3}H, —SO_{v1.3}NH₂, —NHNH₂, —ONH₂, —NHC(O)NHNH₂, —NHC(O)NH₂, —N(O)_{m1.3}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁴ is hydrogen, halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SO_{n1.4}H, —SO_{v1.4}NH₂, —NHNH₂, —ONH₂, —NHC(O)NHNH₂, —NHC(O)NH₂, —N(O)_{m1.4}, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R²⁸ is independently halogen, oxo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —SOₙ₂₈H, —SOᵥ₂₈NH₂, —NHNH₂, —ONH₂, —NHC(O)NHNH₂, —NHC(O)NH₂, —N(O)ₘ₂₈, —NH₂, —C(O)H, —C(O)OH, —C(O)NH₂, —OH, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

60. The compound of claim 1, wherein the compound is:

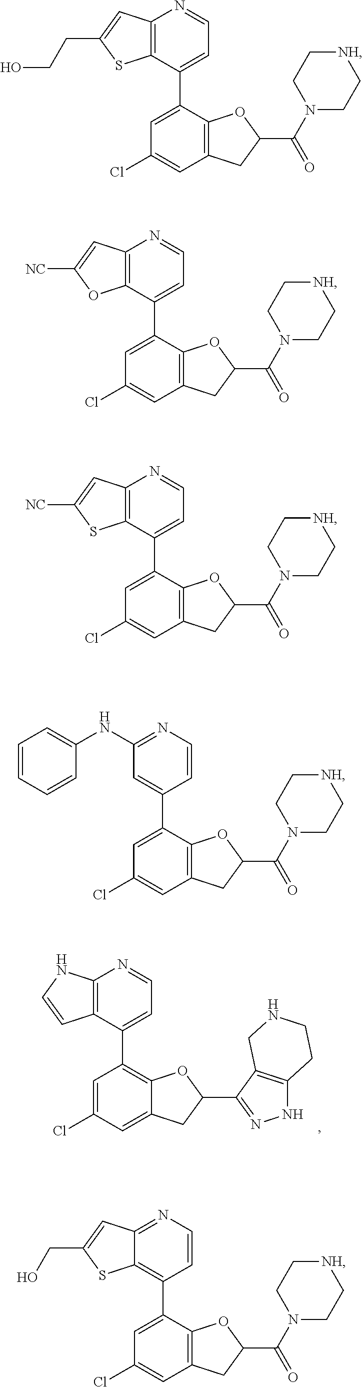 [SMILES: O=C(C1Cc2cc(Cl)cc(-c3ccnc4cc(CCO)sc34)c2O1)N1CCNCC1 ; N#Cc1cc2nccc(-c3cc(Cl)cc4c3OC(C(=O)N3CCNCC3)C4)c2o1 ; N#Cc1cc2nccc(-c3cc(Cl)cc4c3OC(C(=O)N3CCNCC3)C4)c2s1 ; O=C(C1Cc2cc(Cl)cc(-c3ccnc(Nc4ccccc4)c3)c2O1)N1CCNCC1 ; Clc1cc2c(c(-c3ccnc4[nH]ccc34)c1)OC(c1n[nH]c3c1CNCC3)C2 ; O=C(C1Cc2cc(Cl)cc(-c3ccnc4cc(CO)sc34)c2O1)N1CCNCC1]

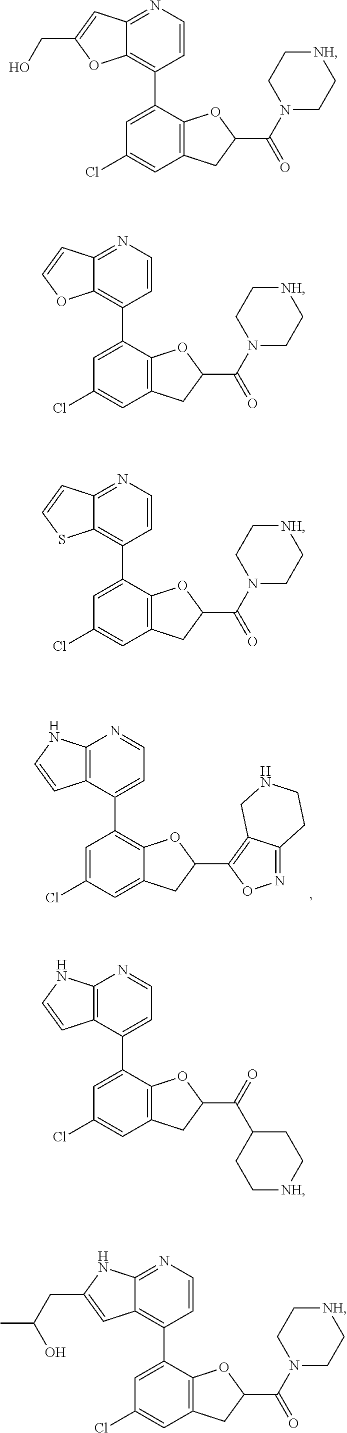 [SMILES: O=C(C1Cc2cc(Cl)cc(-c3ccnc4cc(CO)oc34)c2O1)N1CCNCC1 ; O=C(C1Cc2cc(Cl)cc(-c3ccnc4ccoc34)c2O1)N1CCNCC1 ; O=C(C1Cc2cc(Cl)cc(-c3ccnc4ccsc34)c2O1)N1CCNCC1 ; Clc1cc2c(c(-c3ccnc4[nH]ccc34)c1)OC(c1onc3c1CNCC3)C2 ; O=C(C1CCNCC1)C1Cc2cc(Cl)cc(-c3ccnc4[nH]ccc34)c2O1 ; CC(O)Cc1cc2c(-c3cc(Cl)cc4c3OC(C(=O)N3CCNCC3)C4)ccnc2[nH]1]

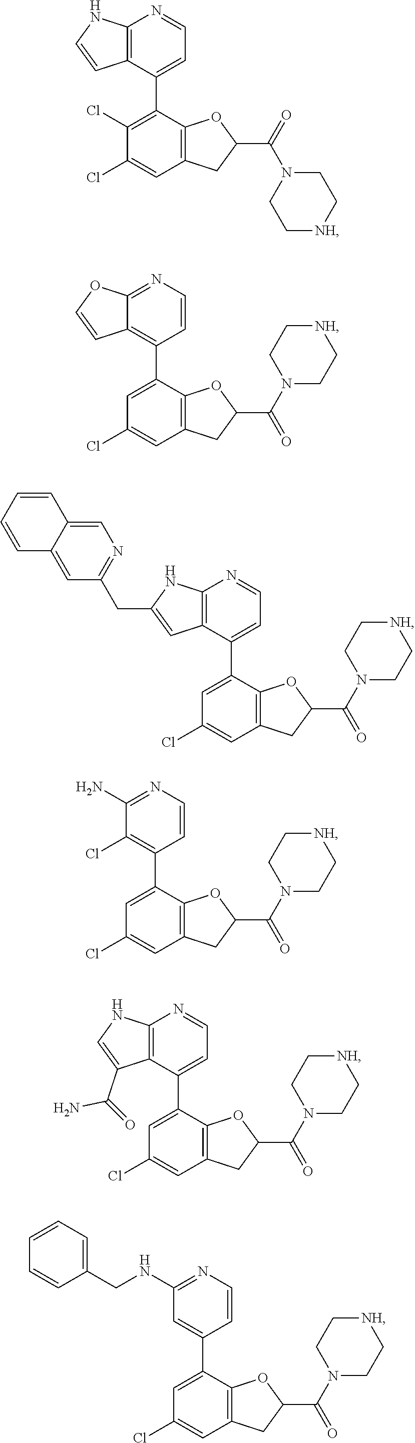 [SMILES: O=C(C1Cc2cc(Cl)c(Cl)c(-c3ccnc4[nH]ccc34)c2O1)N1CCNCC1 ; O=C(C1Cc2cc(Cl)cc(-c3ccnc4occc34)c2O1)N1CCNCC1 ; O=C(C1Cc2cc(Cl)cc(-c3ccnc4[nH]c(Cc5cc6ccccc6cn5)cc34)c2O1)N1CCNCC1 ; Nc1nccc(-c2cc(Cl)cc3c2OC(C(=O)N2CCNCC2)C3)c1Cl ; NC(=O)c1c[nH]c2nccc(-c3cc(Cl)cc4c3OC(C(=O)N3CCNCC3)C4)c12 ; O=C(C1Cc2cc(Cl)cc(-c3ccnc(NCc4ccccc4)c3)c2O1)N1CCNCC1]

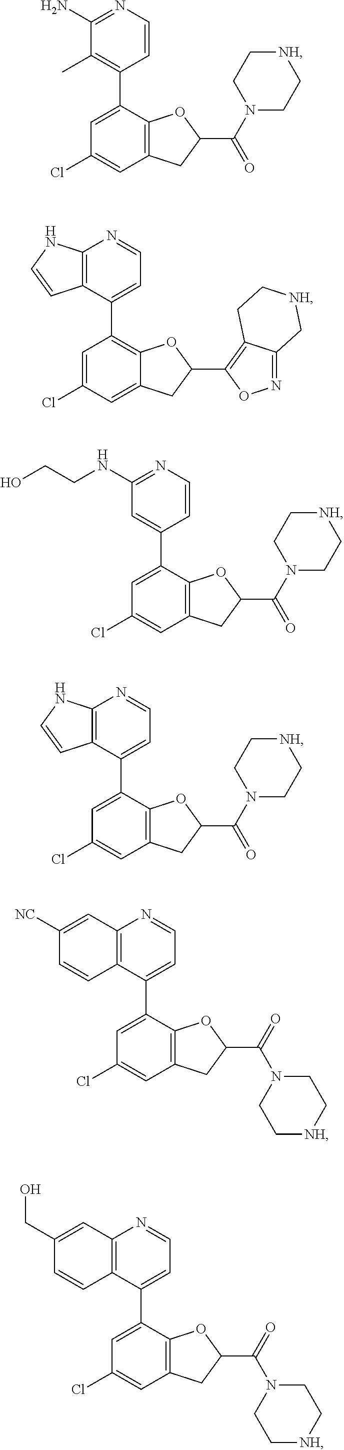 [SMILES: Cc1c(-c2cc(Cl)cc3c2OC(C(=O)N2CCNCC2)C3)ccnc1N ; Clc1cc2c(c(-c3ccnc4[nH]ccc34)c1)OC(c1onc3c1CCNC3)C2 ; O=C(C1Cc2cc(Cl)cc(-c3ccnc(NCCO)c3)c2O1)N1CCNCC1 ; O=C(C1Cc2cc(Cl)cc(-c3ccnc4[nH]ccc34)c2O1)N1CCNCC1 ; N#Cc1ccc2c(-c3cc(Cl)cc4c3OC(C(=O)N3CCNCC3)C4)ccnc2c1 ; O=C(C1Cc2cc(Cl)cc(-c3ccnc4cc(CO)ccc34)c2O1)N1CCNCC1]

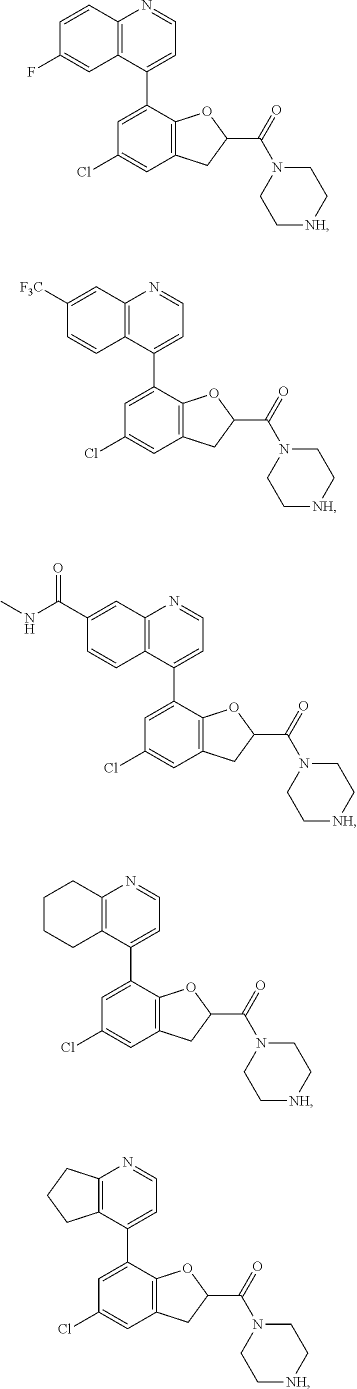 [SMILES: O=C(C1Cc2cc(Cl)cc(-c3ccnc4ccc(F)cc34)c2O1)N1CCNCC1 ; O=C(C1Cc2cc(Cl)cc(-c3ccnc4cc(C(F)(F)F)ccc34)c2O1)N1CCNCC1 ; CNC(=O)c1ccc2c(-c3cc(Cl)cc4c3OC(C(=O)N3CCNCC3)C4)ccnc2c1 ; O=C(C1Cc2cc(Cl)cc(-c3ccnc4c3CCCC4)c2O1)N1CCNCC1 ; O=C(C1Cc2cc(Cl)cc(-c3ccnc4c3CCC4)c2O1)N1CCNCC1]

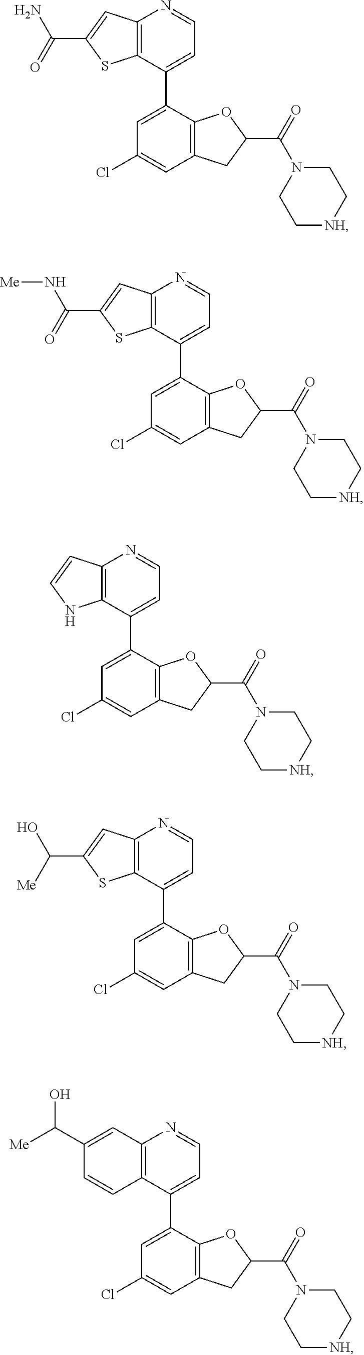 [SMILES: NC(=O)c1cc2nccc(-c3cc(Cl)cc4c3OC(C(=O)N3CCNCC3)C4)c2s1 ; CNC(=O)c1cc2nccc(-c3cc(Cl)cc4c3OC(C(=O)N3CCNCC3)C4)c2s1 ; O=C(C1Cc2cc(Cl)cc(-c3ccnc4cc[nH]c34)c2O1)N1CCNCC1 ; CC(O)c1cc2nccc(-c3cc(Cl)cc4c3OC(C(=O)N3CCNCC3)C4)c2s1 ; CC(O)c1ccc2c(-c3cc(Cl)cc4c3OC(C(=O)N3CCNCC3)C4)ccnc2c1]

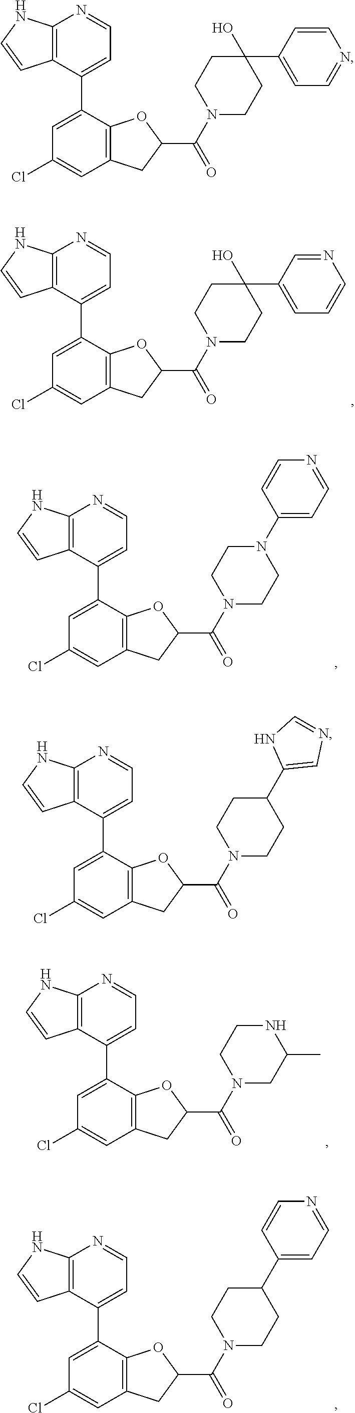 [SMILES: O=C(C1Cc2cc(Cl)cc(-c3ccnc4[nH]ccc34)c2O1)N1CCC(O)(c2ccncc2)CC1 ; O=C(C1Cc2cc(Cl)cc(-c3ccnc4[nH]ccc34)c2O1)N1CCC(O)(c2cccnc2)CC1 ; O=C(C1Cc2cc(Cl)cc(-c3ccnc4[nH]ccc34)c2O1)N1CCN(c2ccncc2)CC1 ; O=C(C1Cc2cc(Cl)cc(-c3ccnc4[nH]ccc34)c2O1)N1CCC(c2cnc[nH]2)CC1 ; CC1CN(C(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(c2ccncc2)CC1]

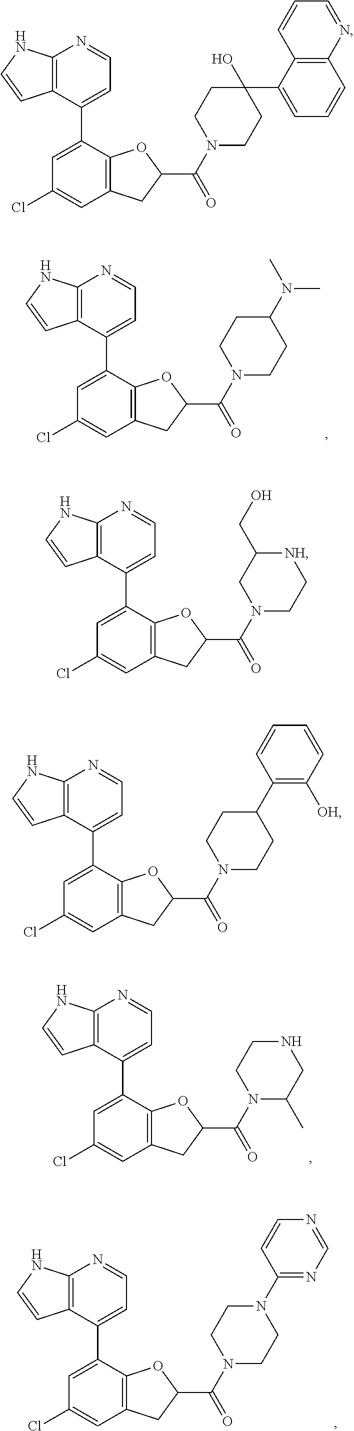 [SMILES: O=C(C1Cc2cc(Cl)cc(-c3ccnc4[nH]ccc34)c2O1)N1CCC(O)(c2cccc3ncccc23)CC1 ; CN(C)C1CCN(C(=O)C2Cc3cc(Cl)cc(-c4ccnc5[nH]ccc45)c3O2)CC1 ; O=C(C1Cc2cc(Cl)cc(-c3ccnc4[nH]ccc34)c2O1)N1CCNC(CO)C1 ; O=C(C1Cc2cc(Cl)cc(-c3ccnc4[nH]ccc34)c2O1)N1CCC(c2ccccc2O)CC1 ; CC1CNCCN1C(=O)C1Cc2cc(Cl)cc(-c3ccnc4[nH]ccc34)c2O1 ; O=C(C1Cc2cc(Cl)cc(-c3ccnc4[nH]ccc34)c2O1)N1CCN(c2ccncn2)CC1]

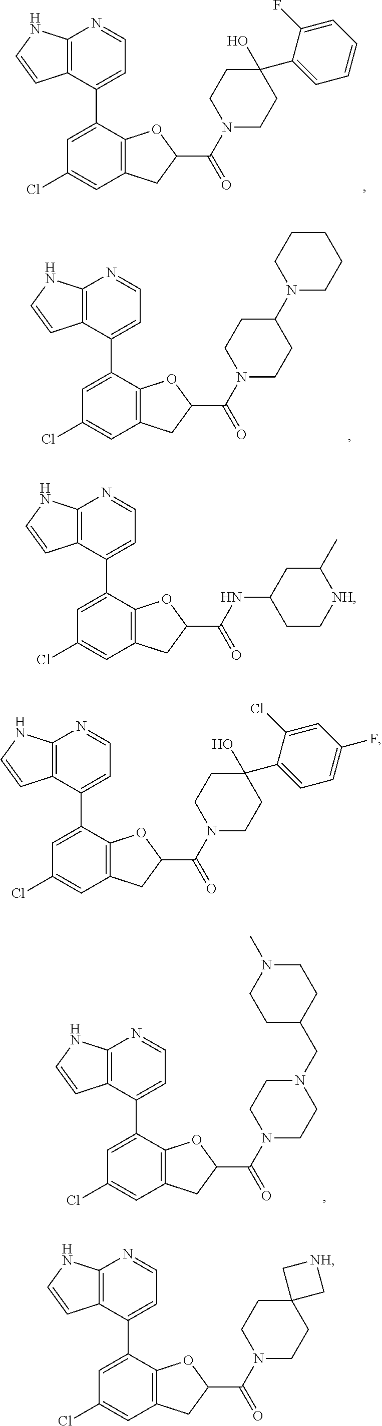 [SMILES: O=C(C1Cc2cc(Cl)cc(-c3ccnc4[nH]ccc34)c2O1)N1CCC(O)(c2ccccc2F)CC1 ; O=C(C1Cc2cc(Cl)cc(-c3ccnc4[nH]ccc34)c2O1)N1CCC(N2CCCCC2)CC1 ; CC1CC(NC(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(O)(c2ccc(F)cc2Cl)CC1 ; CN1CCC(CN2CCN(C(=O)C3Cc4cc(Cl)cc(-c5ccnc6[nH]ccc56)c4O3)CC2)CC1 ; O=C(C1Cc2cc(Cl)cc(-c3ccnc4[nH]ccc34)c2O1)N1CCC2(CC1)CNC2]

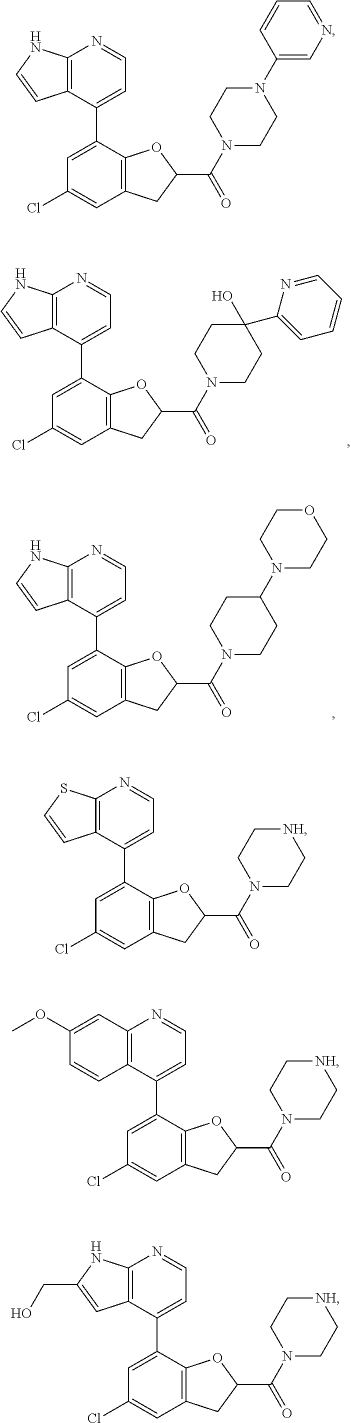 [SMILES: O=C(C1Cc2cc(Cl)cc(-c3ccnc4[nH]ccc34)c2O1)N1CCN(c2cccnc2)CC1 ; O=C(C1Cc2cc(Cl)cc(-c3ccnc4[nH]ccc34)c2O1)N1CCC(O)(c2ccccn2)CC1 ; O=C(C1Cc2cc(Cl)cc(-c3ccnc4[nH]ccc34)c2O1)N1CCC(N2CCOCC2)CC1 ; O=C(C1Cc2cc(Cl)cc(-c3ccnc4sccc34)c2O1)N1CCNCC1 ; COc1ccc2c(-c3cc(Cl)cc4c3OC(C(=O)N3CCNCC3)C4)ccnc2c1 ; O=C(C1Cc2cc(Cl)cc(-c3ccnc4[nH]c(CO)cc34)c2O1)N1CCNCC1]

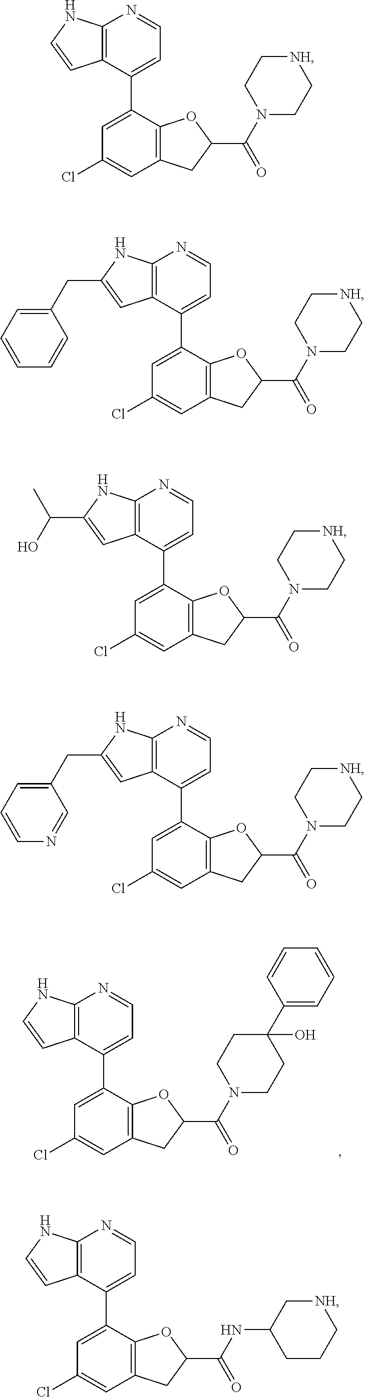 [SMILES: O=C(C1Cc2cc(Cl)cc(-c3ccnc4[nH]ccc34)c2O1)N1CCNCC1 ; O=C(C1Cc2cc(Cl)cc(-c3ccnc4[nH]c(Cc5ccccc5)cc34)c2O1)N1CCNCC1 ; CC(O)c1cc2c(-c3cc(Cl)cc4c3OC(C(=O)N3CCNCC3)C4)ccnc2[nH]1 ; O=C(C1Cc2cc(Cl)cc(-c3ccnc4[nH]c(Cc5cccnc5)cc34)c2O1)N1CCNCC1 ; O=C(C1Cc2cc(Cl)cc(-c3ccnc4[nH]ccc34)c2O1)N1CCC(O)(c2ccccc2)CC1 ; O=C(NC1CCCNC1)C1Cc2cc(Cl)cc(-c3ccnc4[nH]ccc34)c2O1]

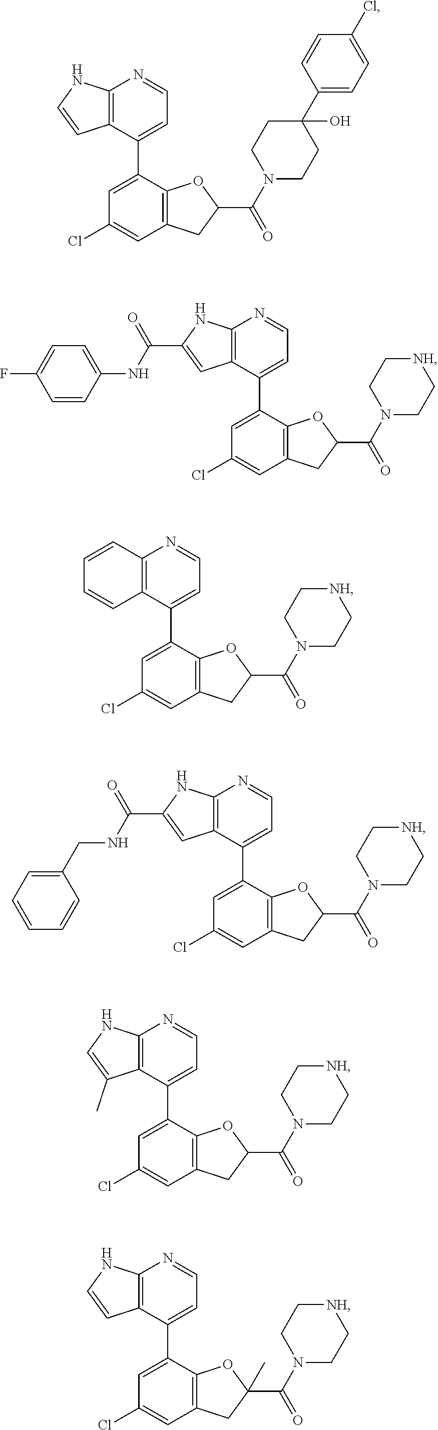 [SMILES: O=C(C1Cc2cc(Cl)cc(-c3ccnc4[nH]ccc34)c2O1)N1CCC(O)(c2ccc(Cl)cc2)CC1 ; O=C(Nc1ccc(F)cc1)c1cc2c(-c3cc(Cl)cc4c3OC(C(=O)N3CCNCC3)C4)ccnc2[nH]1 ; O=C(C1Cc2cc(Cl)cc(-c3ccnc4ccccc34)c2O1)N1CCNCC1 ; O=C(NCc1ccccc1)c1cc2c(-c3cc(Cl)cc4c3OC(C(=O)N3CCNCC3)C4)ccnc2[nH]1 ; Cc1c[nH]c2nccc(-c3cc(Cl)cc4c3OC(C(=O)N3CCNCC3)C4)c12 ; CC1(C(=O)N2CCNCC2)Cc2cc(Cl)cc(-c3ccnc4[nH]ccc34)c2O1]

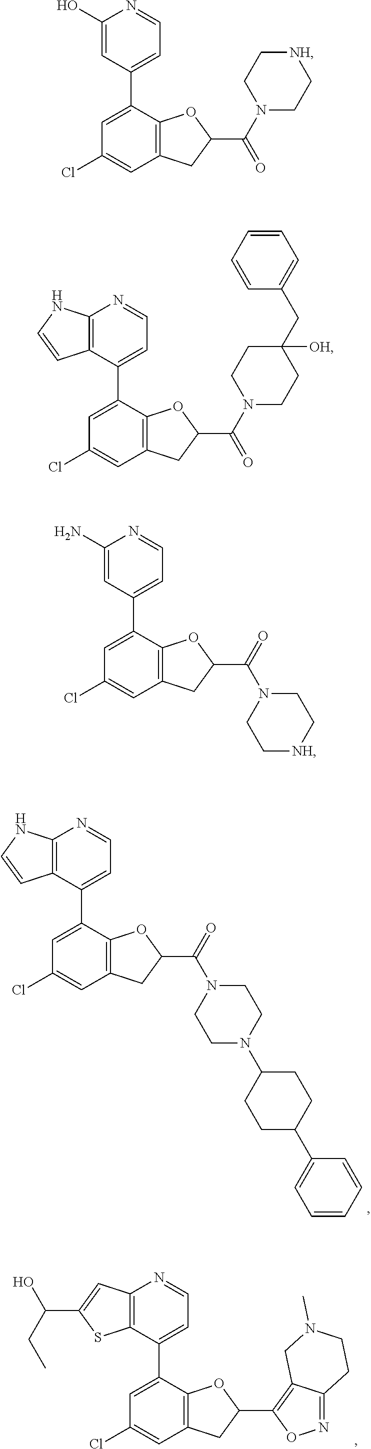 [SMILES: O=C(C1Cc2cc(Cl)cc(-c3ccnc(O)c3)c2O1)N1CCNCC1 ; O=C(C1Cc2cc(Cl)cc(-c3ccnc4[nH]ccc34)c2O1)N1CCC(O)(Cc2ccccc2)CC1 ; Nc1cc(-c2cc(Cl)cc3c2OC(C(=O)N2CCNCC2)C3)ccn1 ; O=C(C1Cc2cc(Cl)cc(-c3ccnc4[nH]ccc34)c2O1)N1CCN(C2CCC(c3ccccc3)CC2)CC1 ; CCC(O)c1cc2nccc(-c3cc(Cl)cc4c3OC(c3onc5c3CN(C)CC5)C4)c2s1]

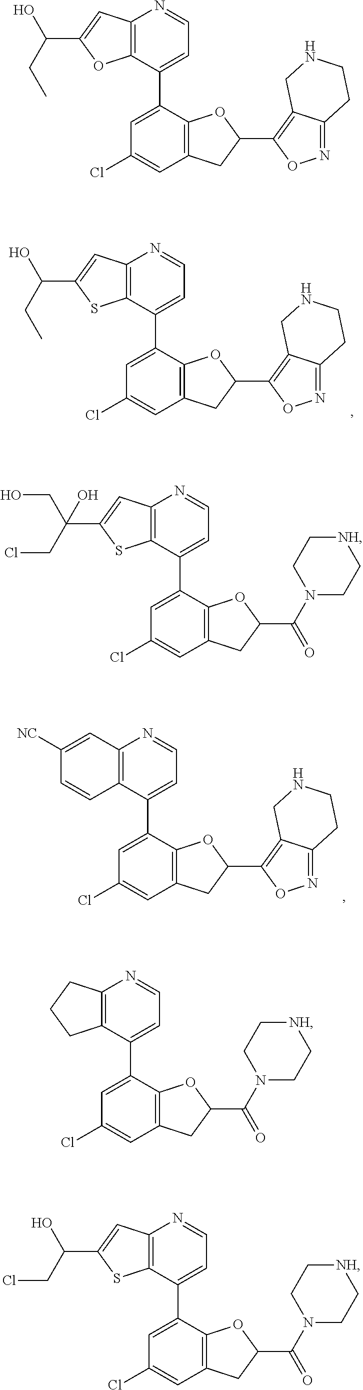 [SMILES: CCC(O)c1cc2nccc(-c3cc(Cl)cc4c3OC(c3onc5c3CNCC5)C4)c2o1 ; CCC(O)c1cc2nccc(-c3cc(Cl)cc4c3OC(c3onc5c3CNCC5)C4)c2s1 ; O=C(C1Cc2cc(Cl)cc(-c3ccnc4cc(C(O)(CO)CCl)sc34)c2O1)N1CCNCC1 ; N#Cc1ccc2c(-c3cc(Cl)cc4c3OC(c3onc5c3CNCC5)C4)ccnc2c1 ; O=C(C1Cc2cc(Cl)cc(-c3ccnc4c3CCC4)c2O1)N1CCNCC1 ; O=C(C1Cc2cc(Cl)cc(-c3ccnc4cc(C(O)CCl)sc34)c2O1)N1CCNCC1]

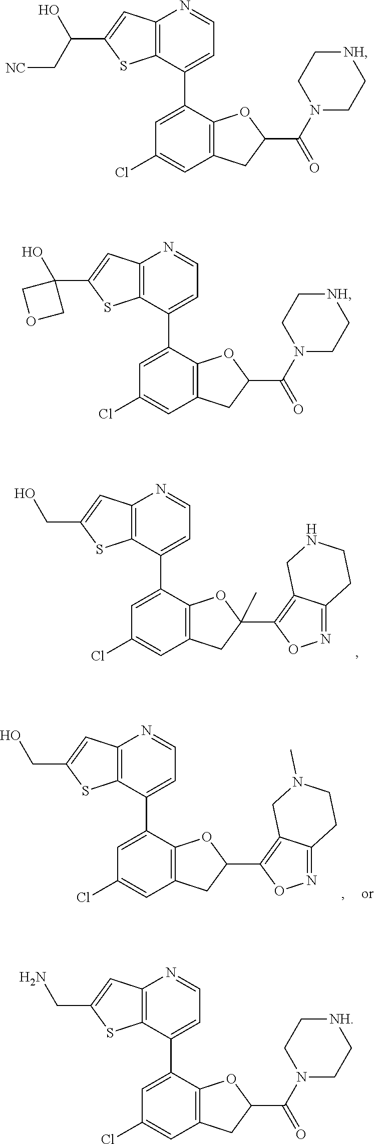 [SMILES: N#CCC(O)c1cc2nccc(-c3cc(Cl)cc4c3OC(C(=O)N3CCNCC3)C4)c2s1 ; O=C(C1Cc2cc(Cl)cc(-c3ccnc4cc(C5(O)COC5)sc34)c2O1)N1CCNCC1 ; CC1(c2onc3c2CNCC3)Cc2cc(Cl)cc(-c3ccnc4cc(CO)sc34)c2O1 ; CN1CCc2noc(C3Cc4cc(Cl)cc(-c5ccnc6cc(CO)sc56)c4O3)c2C1 ; NCc1cc2nccc(-c3cc(Cl)cc4c3OC(C(=O)N3CCNCC3)C4)c2s1]

61. The compound of claim 1, wherein the compound is

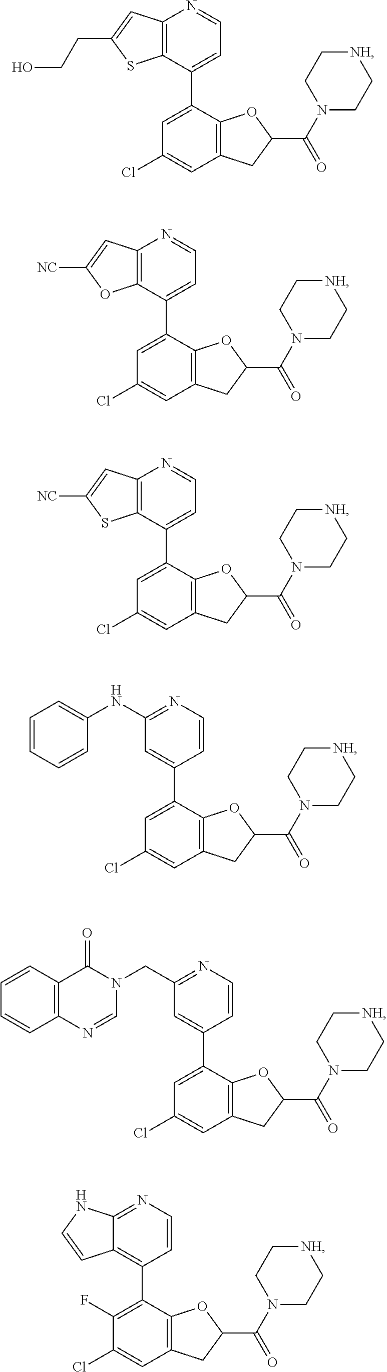 [SMILES: O=C(C1Cc2cc(Cl)cc(-c3ccnc4cc(CCO)sc34)c2O1)N1CCNCC1 ; N#Cc1cc2nccc(-c3cc(Cl)cc4c3OC(C(=O)N3CCNCC3)C4)c2o1 ; N#Cc1cc2nccc(-c3cc(Cl)cc4c3OC(C(=O)N3CCNCC3)C4)c2s1 ; O=C(C1Cc2cc(Cl)cc(-c3ccnc(Nc4ccccc4)c3)c2O1)N1CCNCC1 ; O=C(C1Cc2cc(Cl)cc(-c3ccnc(Cn4cnc5ccccc5c4=O)c3)c2O1)N1CCNCC1 ; O=C(C1Cc2cc(Cl)c(F)c(-c3ccnc4[nH]ccc34)c2O1)N1CCNCC1]

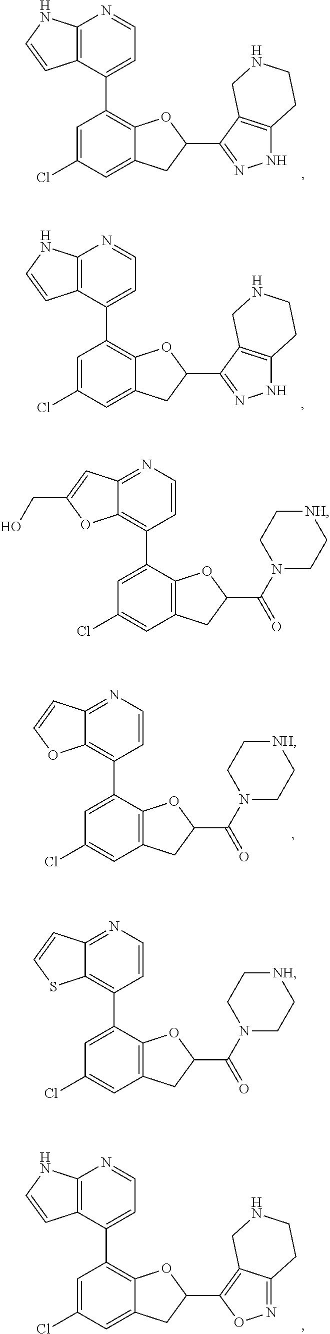 [SMILES: Clc1cc2c(c(-c3ccnc4[nH]ccc34)c1)OC(c1n[nH]c3c1CNCC3)C2 ; Clc1cc2c(c(-c3ccnc4[nH]ccc34)c1)OC(c1n[nH]c3c1CNCC3)C2 ; O=C(C1Cc2cc(Cl)cc(-c3ccnc4cc(CO)oc34)c2O1)N1CCNCC1 ; O=C(C1Cc2cc(Cl)cc(-c3ccnc4ccoc34)c2O1)N1CCNCC1 ; O=C(C1Cc2cc(Cl)cc(-c3ccnc4ccsc34)c2O1)N1CCNCC1 ; Clc1cc2c(c(-c3ccnc4[nH]ccc34)c1)OC(c1onc3c1CNCC3)C2]

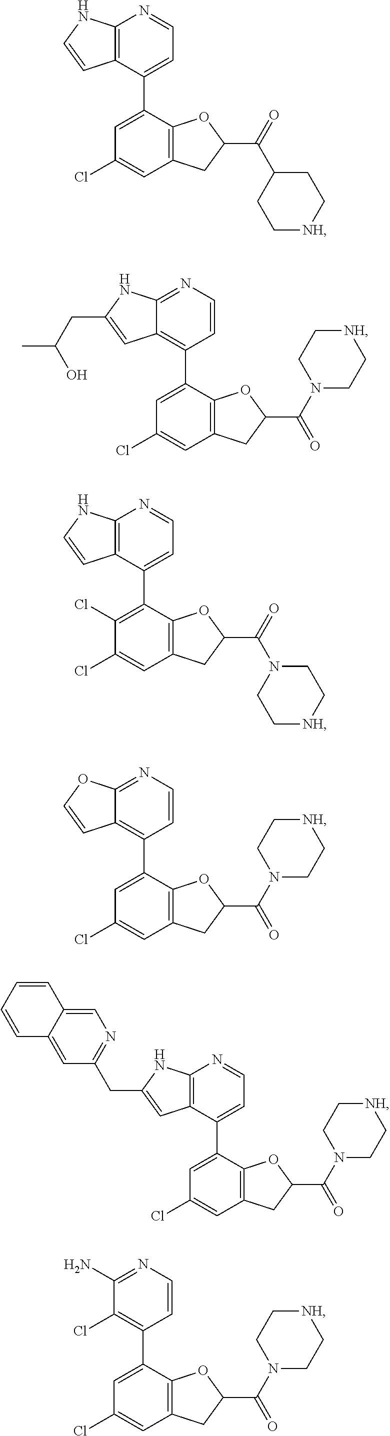 [SMILES: O=C(C1CCNCC1)C1Cc2cc(Cl)cc(-c3ccnc4[nH]ccc34)c2O1 ; CC(O)Cc1cc2c(-c3cc(Cl)cc4c3OC(C(=O)N3CCNCC3)C4)ccnc2[nH]1 ; O=C(C1Cc2cc(Cl)c(Cl)c(-c3ccnc4[nH]ccc34)c2O1)N1CCNCC1 ; O=C(C1Cc2cc(Cl)cc(-c3ccnc4occc34)c2O1)N1CCNCC1 ; O=C(C1Cc2cc(Cl)cc(-c3ccnc4[nH]c(Cc5cc6ccccc6cn5)cc34)c2O1)N1CCNCC1 ; Nc1nccc(-c2cc(Cl)cc3c2OC(C(=O)N2CCNCC2)C3)c1Cl]

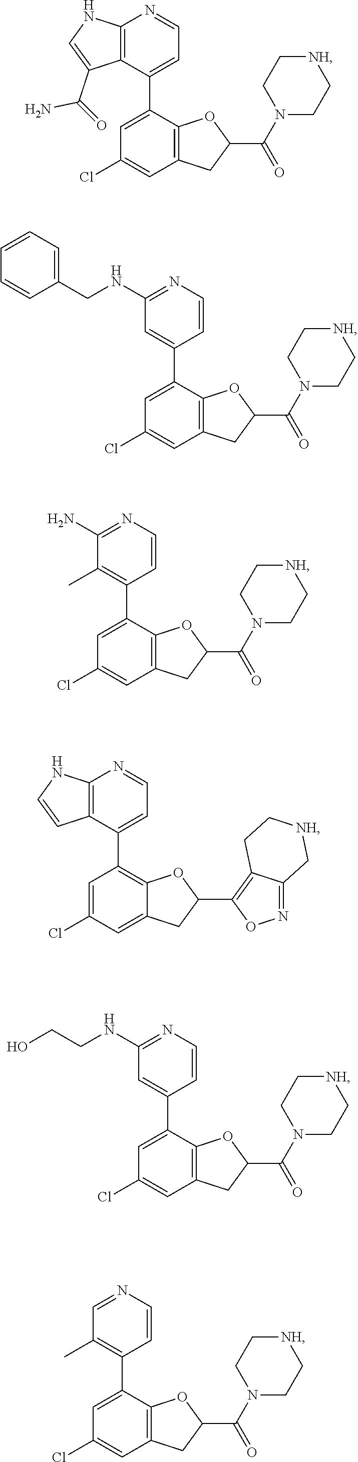 [SMILES: NC(=O)c1c[nH]c2nccc(-c3cc(Cl)cc4c3OC(C(=O)N3CCNCC3)C4)c12 ; O=C(C1Cc2cc(Cl)cc(-c3ccnc(NCc4ccccc4)c3)c2O1)N1CCNCC1 ; Cc1c(-c2cc(Cl)cc3c2OC(C(=O)N2CCNCC2)C3)ccnc1N ; Clc1cc2c(c(-c3ccnc4[nH]ccc34)c1)OC(c1onc3c1CCNC3)C2 ; O=C(C1Cc2cc(Cl)cc(-c3ccnc(NCCO)c3)c2O1)N1CCNCC1 ; Cc1cnccc1-c1cc(Cl)cc2c1OC(C(=O)N1CCNCC1)C2]

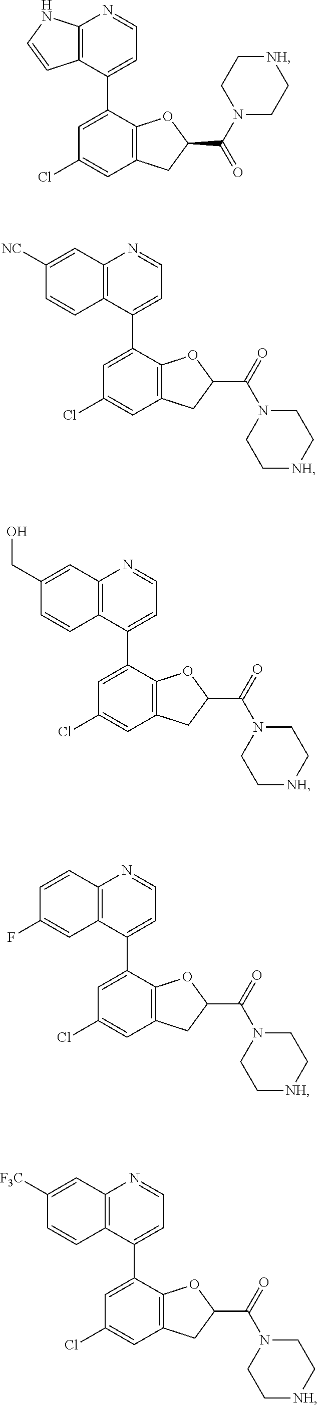 [SMILES: O=C([C@H]1Cc2cc(Cl)cc(-c3ccnc4[nH]ccc34)c2O1)N1CCNCC1 ; N#Cc1ccc2c(-c3cc(Cl)cc4c3OC(C(=O)N3CCNCC3)C4)ccnc2c1 ; O=C(C1Cc2cc(Cl)cc(-c3ccnc4cc(CO)ccc34)c2O1)N1CCNCC1 ; O=C(C1Cc2cc(Cl)cc(-c3ccnc4ccc(F)cc34)c2O1)N1CCNCC1 ; O=C(C1Cc2cc(Cl)cc(-c3ccnc4cc(C(F)(F)F)ccc34)c2O1)N1CCNCC1]

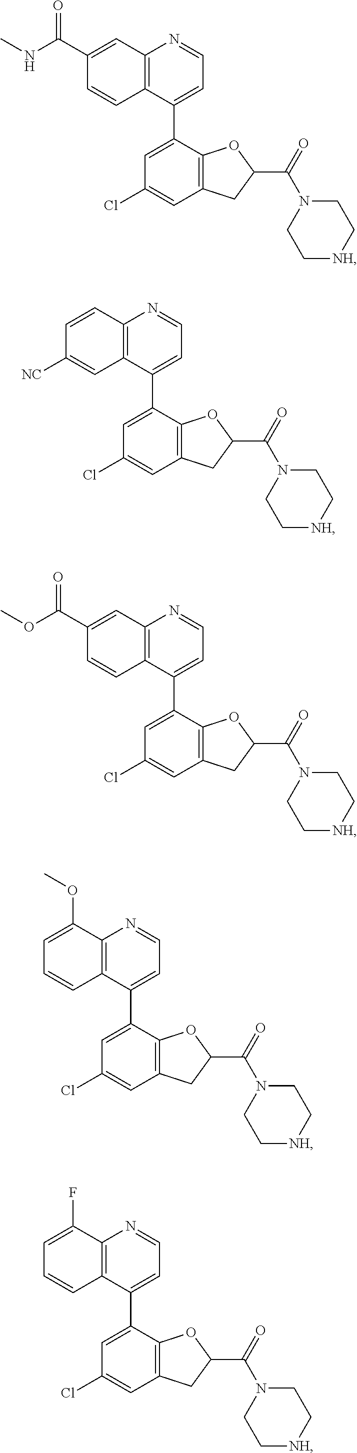 [SMILES: CNC(=O)c1ccc2c(-c3cc(Cl)cc4c3OC(C(=O)N3CCNCC3)C4)ccnc2c1 ; N#Cc1ccc2nccc(-c3cc(Cl)cc4c3OC(C(=O)N3CCNCC3)C4)c2c1 ; COC(=O)c1ccc2c(-c3cc(Cl)cc4c3OC(C(=O)N3CCNCC3)C4)ccnc2c1 ; COc1cccc2c(-c3cc(Cl)cc4c3OC(C(=O)N3CCNCC3)C4)ccnc12 ; O=C(C1Cc2cc(Cl)cc(-c3ccnc4c(F)cccc34)c2O1)N1CCNCC1]

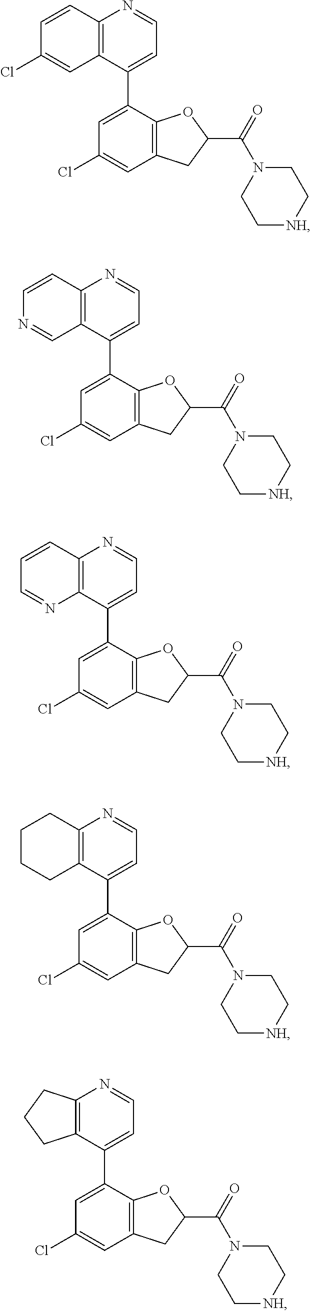 [SMILES: O=C(C1Cc2cc(Cl)cc(-c3ccnc4ccc(Cl)cc34)c2O1)N1CCNCC1 ; O=C(C1Cc2cc(Cl)cc(-c3ccnc4ccncc34)c2O1)N1CCNCC1 ; O=C(C1Cc2cc(Cl)cc(-c3ccnc4cccnc34)c2O1)N1CCNCC1 ; O=C(C1Cc2cc(Cl)cc(-c3ccnc4c3CCCC4)c2O1)N1CCNCC1 ; O=C(C1Cc2cc(Cl)cc(-c3ccnc4c3CCC4)c2O1)N1CCNCC1]

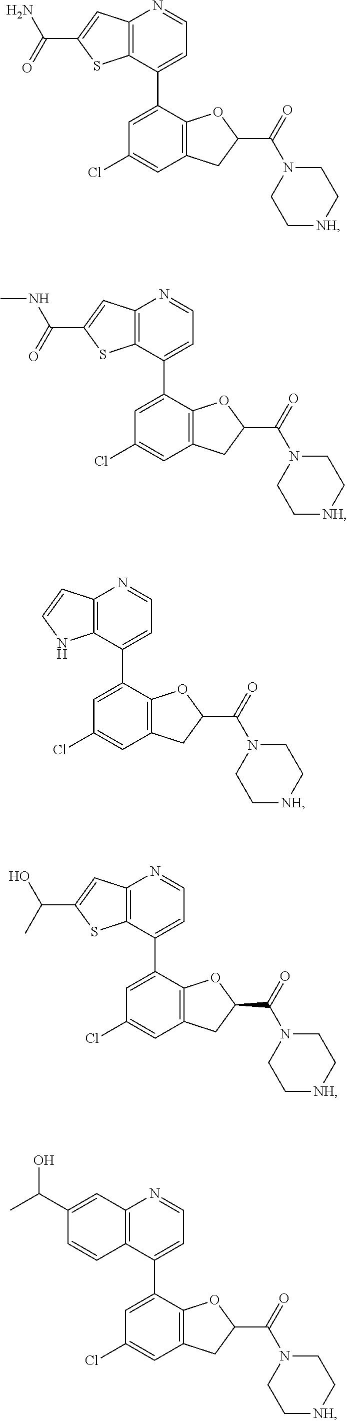 [SMILES: NC(=O)c1cc2nccc(-c3cc(Cl)cc4c3OC(C(=O)N3CCNCC3)C4)c2s1 ; CNC(=O)c1cc2nccc(-c3cc(Cl)cc4c3OC(C(=O)N3CCNCC3)C4)c2s1 ; O=C(C1Cc2cc(Cl)cc(-c3ccnc4cc[nH]c34)c2O1)N1CCNCC1 ; CC(O)c1cc2nccc(-c3cc(Cl)cc4c3O[C@@H](C(=O)N3CCNCC3)C4)c2s1 ; CC(O)c1ccc2c(-c3cc(Cl)cc4c3OC(C(=O)N3CCNCC3)C4)ccnc2c1]

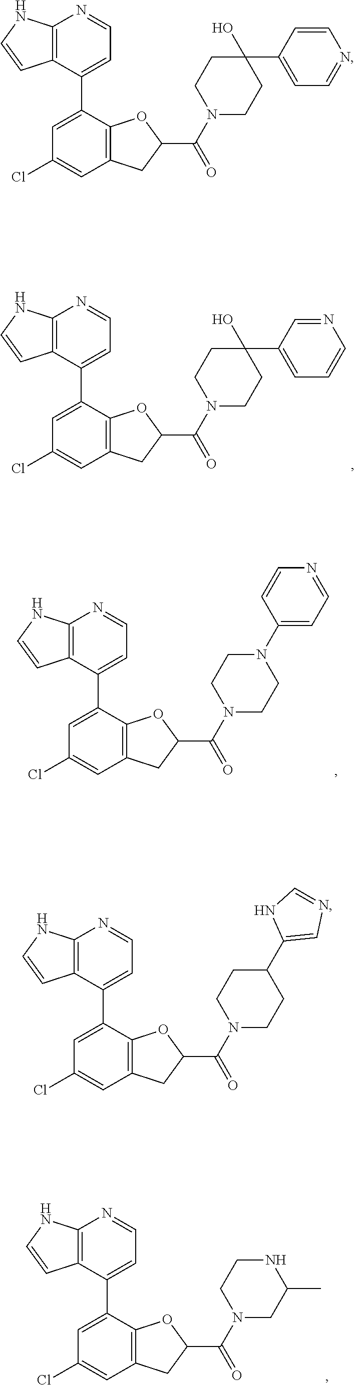 [SMILES: O=C(C1Cc2cc(Cl)cc(-c3ccnc4[nH]ccc34)c2O1)N1CCC(O)(c2ccncc2)CC1 ; O=C(C1Cc2cc(Cl)cc(-c3ccnc4[nH]ccc34)c2O1)N1CCC(O)(c2cccnc2)CC1 ; O=C(C1Cc2cc(Cl)cc(-c3ccnc4[nH]ccc34)c2O1)N1CCN(c2ccncc2)CC1 ; O=C(C1Cc2cc(Cl)cc(-c3ccnc4[nH]ccc34)c2O1)N1CCC(c2cnc[nH]2)CC1 ; CC1CN(C(=O)C2Cc3cc(Cl)cc(-c4ccnc5[nH]ccc45)c3O2)CCN1]

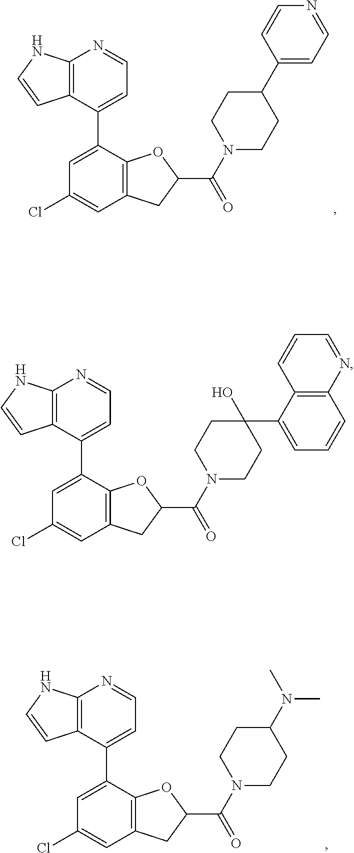 [SMILES: O=C(C1Cc2cc(Cl)cc(-c3ccnc4[nH]ccc34)c2O1)N1CCC(c2ccncc2)CC1 ; O=C(C1Cc2cc(Cl)cc(-c3ccnc4[nH]ccc34)c2O1)N1CCC(O)(c2cccc3ncccc23)CC1 ; CN(C)C1CCN(C(=O)C2Cc3cc(Cl)cc(-c4ccnc5[nH]ccc45)c3O2)CC1]

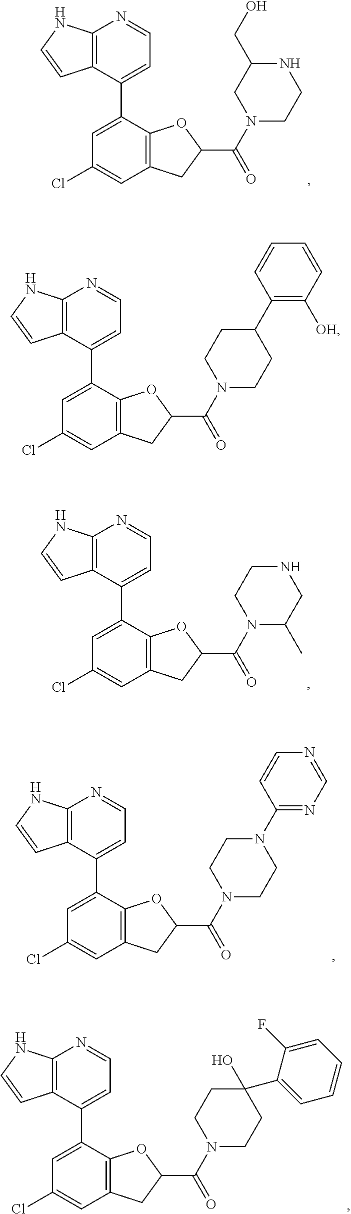 [SMILES: O=C(C1Cc2cc(Cl)cc(-c3ccnc4[nH]ccc34)c2O1)N1CCNC(CO)C1 ; O=C(C1Cc2cc(Cl)cc(-c3ccnc4[nH]ccc34)c2O1)N1CCC(c2ccccc2O)CC1 ; CC1CNCCN1C(=O)C1Cc2cc(Cl)cc(-c3ccnc4[nH]ccc34)c2O1 ; O=C(C1Cc2cc(Cl)cc(-c3ccnc4[nH]ccc34)c2O1)N1CCN(c2ccncn2)CC1 ; O=C(C1Cc2cc(Cl)cc(-c3ccnc4[nH]ccc34)c2O1)N1CCC(O)(c2ccccc2F)CC1]

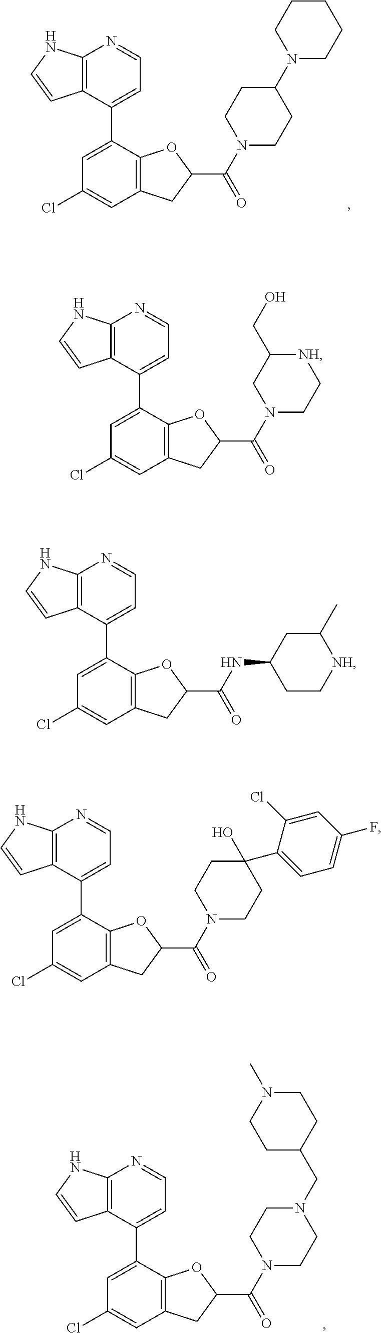 [SMILES: O=C(C1Cc2cc(Cl)cc(-c3ccnc4[nH]ccc34)c2O1)N1CCC(N2CCCCC2)CC1 ; O=C(C1Cc2cc(Cl)cc(-c3ccnc4[nH]ccc34)c2O1)N1CCNC(CO)C1 ; CC1C[C@H](NC(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(O)(c2ccc(F)cc2Cl)CC1 ; CN1CCC(CN2CCN(C(=O)C3Cc4cc(Cl)cc(-c5ccnc6[nH]ccc56)c4O3)CC2)CC1]

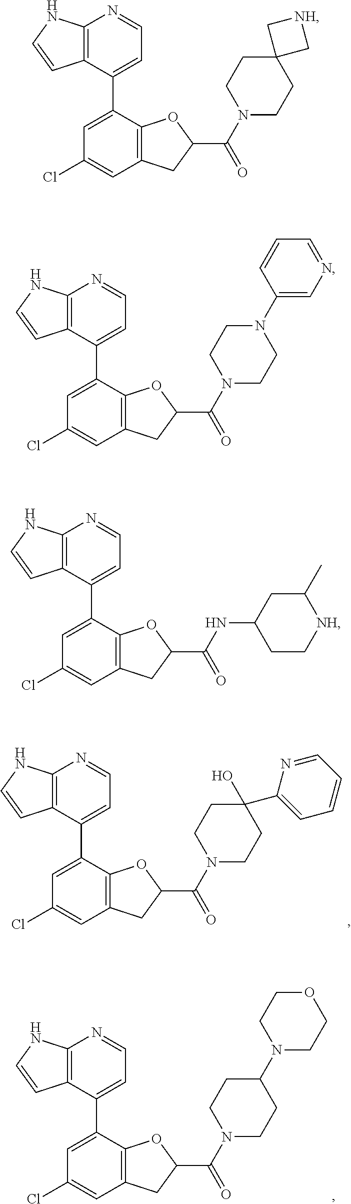 [SMILES: O=C(C1Cc2cc(Cl)cc(-c3ccnc4[nH]ccc34)c2O1)N1CCC2(CC1)CNC2 ; O=C(C1Cc2cc(Cl)cc(-c3ccnc4[nH]ccc34)c2O1)N1CCN(c2cccnc2)CC1 ; CC1CC(NC(=O)C2Cc3cc(Cl)cc(-c4ccnc5[nH]ccc45)c3O2)CCN1 ; O=C(C1Cc2cc(Cl)cc(-c3ccnc4[nH]ccc34)c2O1)N1CCC(O)(c2ccccn2)CC1 ; O=C(C1Cc2cc(Cl)cc(-c3ccnc4[nH]ccc34)c2O1)N1CCC(N2CCOCC2)CC1]

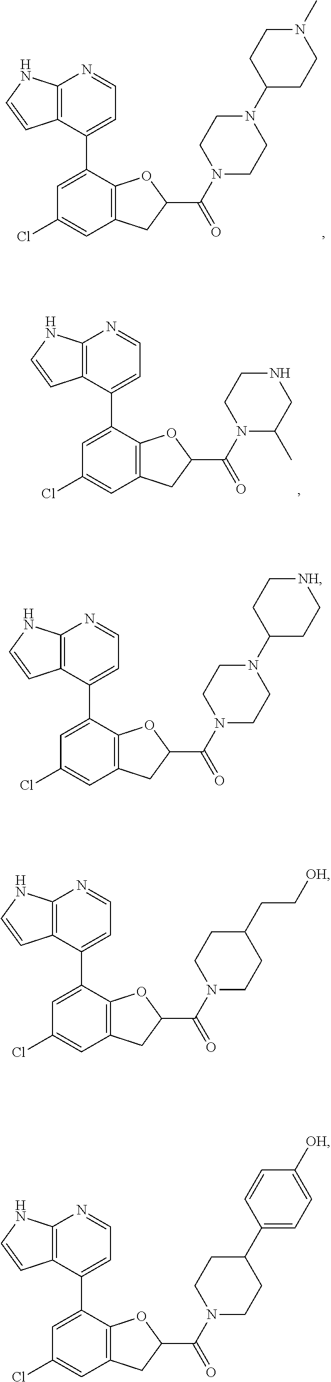 [SMILES: CN1CCC(N2CCN(C(=O)C3Cc4cc(Cl)cc(-c5ccnc6[nH]ccc56)c4O3)CC2)CC1 ; CC1CNCCN1C(=O)C1Cc2cc(Cl)cc(-c3ccnc4[nH]ccc34)c2O1 ; O=C(C1Cc2cc(Cl)cc(-c3ccnc4[nH]ccc34)c2O1)N1CCN(C2CCNCC2)CC1 ; O=C(C1Cc2cc(Cl)cc(-c3ccnc4[nH]ccc34)c2O1)N1CCC(CCO)CC1 ; O=C(C1Cc2cc(Cl)cc(-c3ccnc4[nH]ccc34)c2O1)N1CCC(c2ccc(O)cc2)CC1]

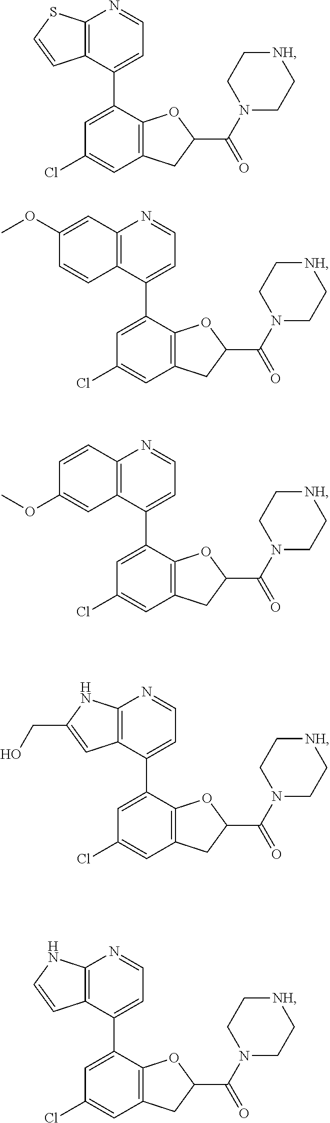 [SMILES: O=C(C1Cc2cc(Cl)cc(-c3ccnc4sccc34)c2O1)N1CCNCC1 ; COc1ccc2c(-c3cc(Cl)cc4c3OC(C(=O)N3CCNCC3)C4)ccnc2c1 ; COc1ccc2nccc(-c3cc(Cl)cc4c3OC(C(=O)N3CCNCC3)C4)c2c1 ; O=C(C1Cc2cc(Cl)cc(-c3ccnc4[nH]c(CO)cc34)c2O1)N1CCNCC1 ; O=C(C1Cc2cc(Cl)cc(-c3ccnc4[nH]ccc34)c2O1)N1CCNCC1]

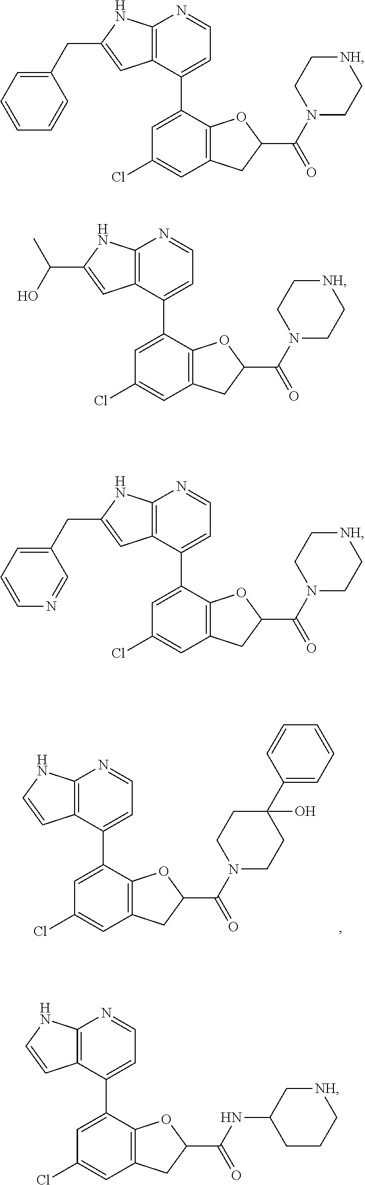 [SMILES: O=C(C1Cc2cc(Cl)cc(-c3ccnc4[nH]c(Cc5ccccc5)cc34)c2O1)N1CCNCC1 ; CC(O)c1cc2c(-c3cc(Cl)cc4c3OC(C(=O)N3CCNCC3)C4)ccnc2[nH]1 ; O=C(C1Cc2cc(Cl)cc(-c3ccnc4[nH]c(Cc5cccnc5)cc34)c2O1)N1CCNCC1 ; O=C(C1Cc2cc(Cl)cc(-c3ccnc4[nH]ccc34)c2O1)N1CCC(O)(c2ccccc2)CC1 ; O=C(NC1CCCNC1)C1Cc2cc(Cl)cc(-c3ccnc4[nH]ccc34)c2O1]

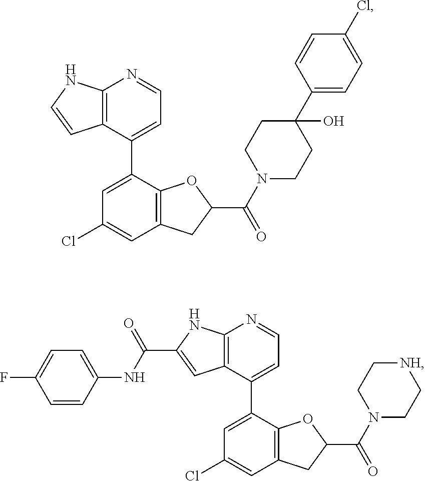 [SMILES: O=C(C1Cc2cc(Cl)cc(-c3ccnc4[nH]ccc34)c2O1)N1CCC(O)(c2ccc(Cl)cc2)CC1 ; O=C(Nc1ccc(F)cc1)c1cc2c(-c3cc(Cl)cc4c3OC(C(=O)N3CCNCC3)C4)ccnc2[nH]1]

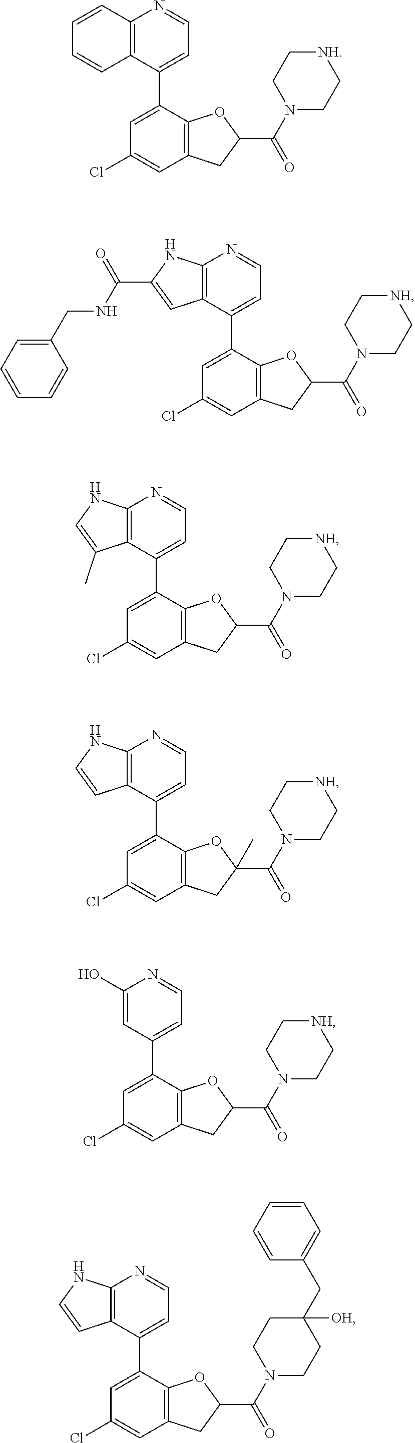 [SMILES: O=C(C1Cc2cc(Cl)cc(-c3ccnc4ccccc34)c2O1)N1CCNCC1 ; O=C(NCc1ccccc1)c1cc2c(-c3cc(Cl)cc4c3OC(C(=O)N3CCNCC3)C4)ccnc2[nH]1 ; Cc1c[nH]c2nccc(-c3cc(Cl)cc4c3OC(C(=O)N3CCNCC3)C4)c12 ; CC1(C(=O)N2CCNCC2)Cc2cc(Cl)cc(-c3ccnc4[nH]ccc34)c2O1 ; O=C(C1Cc2cc(Cl)cc(-c3ccnc(O)c3)c2O1)N1CCNCC1 ; O=C(C1Cc2cc(Cl)cc(-c3ccnc4[nH]ccc34)c2O1)N1CCC(O)(Cc2ccccc2)CC1]

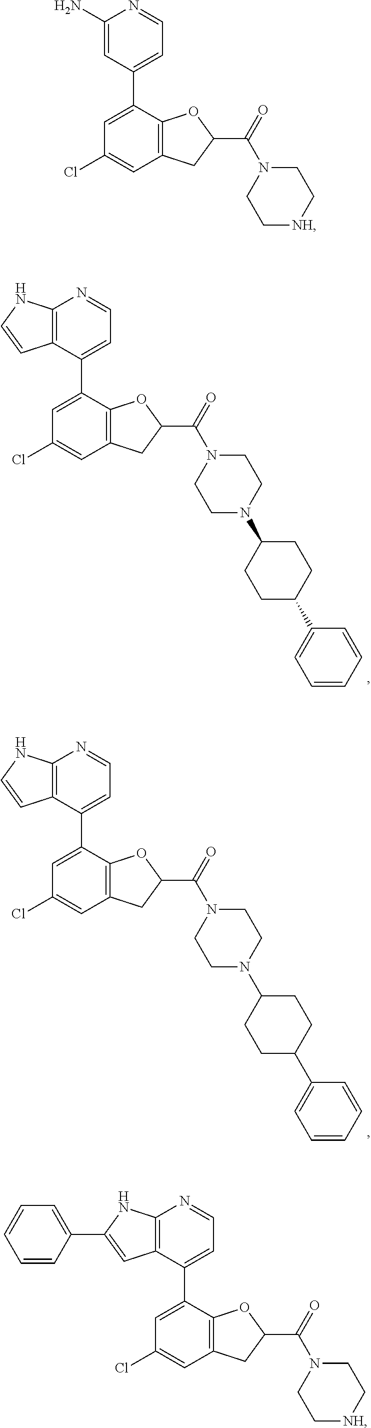 [SMILES: Nc1cc(-c2cc(Cl)cc3c2OC(C(=O)N2CCNCC2)C3)ccn1 ; O=C(C1Cc2cc(Cl)cc(-c3ccnc4[nH]ccc34)c2O1)N1CCN([C@H]2CC[C@H](c3ccccc3)CC2)CC1 ; O=C(C1Cc2cc(Cl)cc(-c3ccnc4[nH]ccc34)c2O1)N1CCN(C2CCC(c3ccccc3)CC2)CC1 ; O=C(C1Cc2cc(Cl)cc(-c3ccnc4[nH]c(-c5ccccc5)cc34)c2O1)N1CCNCC1]

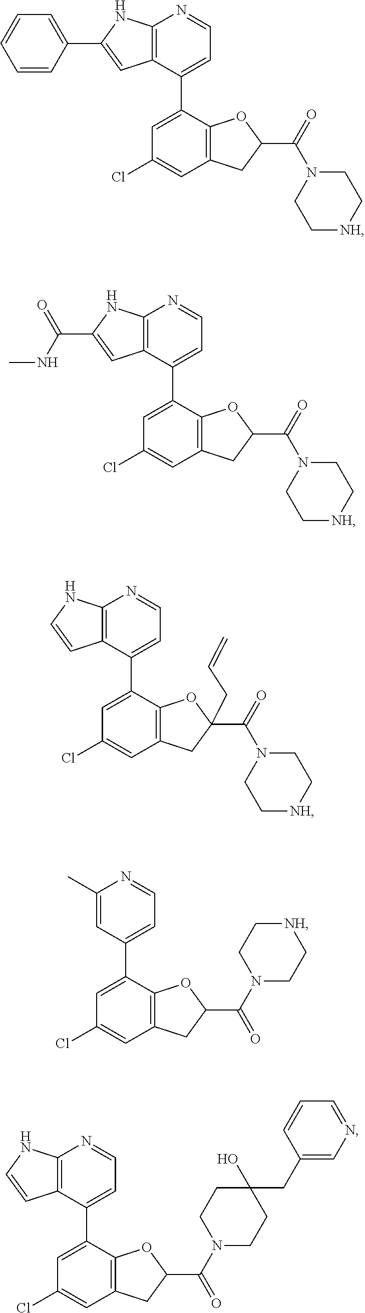 [SMILES: O=C(C1Cc2cc(Cl)cc(-c3ccnc4[nH]c(-c5ccccc5)cc34)c2O1)N1CCNCC1 ; CNC(=O)c1cc2c(-c3cc(Cl)cc4c3OC(C(=O)N3CCNCC3)C4)ccnc2[nH]1 ; C=CCC1(C(=O)N2CCNCC2)Cc2cc(Cl)cc(-c3ccnc4[nH]ccc34)c2O1 ; Cc1cc(-c2cc(Cl)cc3c2OC(C(=O)N2CCNCC2)C3)ccn1 ; O=C(C1Cc2cc(Cl)cc(-c3ccnc4[nH]ccc34)c2O1)N1CCC(O)(Cc2cccnc2)CC1]

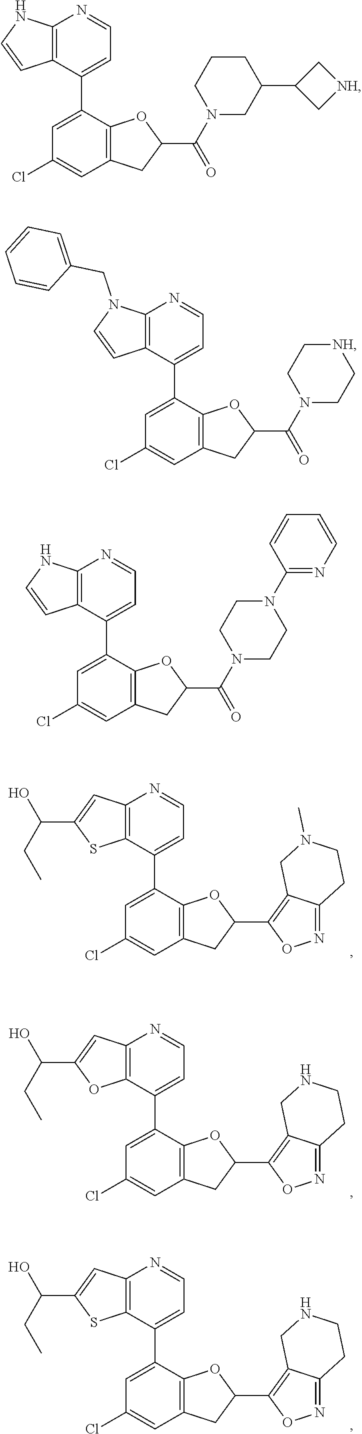 [SMILES: O=C(C1Cc2cc(Cl)cc(-c3ccnc4[nH]ccc34)c2O1)N1CCCC(C2CNC2)C1 ; O=C(C1Cc2cc(Cl)cc(-c3ccnc4c3ccn4Cc3ccccc3)c2O1)N1CCNCC1 ; O=C(C1Cc2cc(Cl)cc(-c3ccnc4[nH]ccc34)c2O1)N1CCN(c2ccccn2)CC1 ; CCC(O)c1cc2nccc(-c3cc(Cl)cc4c3OC(c3onc5c3CN(C)CC5)C4)c2s1 ; CCC(O)c1cc2nccc(-c3cc(Cl)cc4c3OC(c3onc5c3CNCC5)C4)c2o1 ; CCC(O)c1cc2nccc(-c3cc(Cl)cc4c3OC(c3onc5c3CNCC5)C4)c2s1]

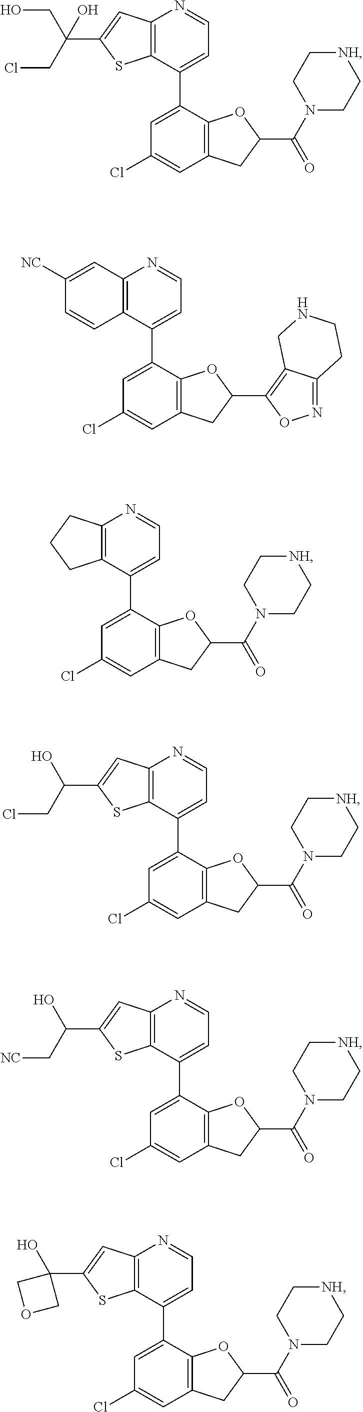 [SMILES: O=C(C1Cc2cc(Cl)cc(-c3ccnc4cc(C(O)(CO)CCl)sc34)c2O1)N1CCNCC1 ; N#Cc1ccc2c(-c3cc(Cl)cc4c3OC(c3onc5c3CNCC5)C4)ccnc2c1 ; O=C(C1Cc2cc(Cl)cc(-c3ccnc4c3CCC4)c2O1)N1CCNCC1 ; O=C(C1Cc2cc(Cl)cc(-c3ccnc4cc(C(O)CCl)sc34)c2O1)N1CCNCC1 ; N#CCC(O)c1cc2nccc(-c3cc(Cl)cc4c3OC(C(=O)N3CCNCC3)C4)c2s1 ; O=C(C1Cc2cc(Cl)cc(-c3ccnc4cc(C5(O)COC5)sc34)c2O1)N1CCNCC1]

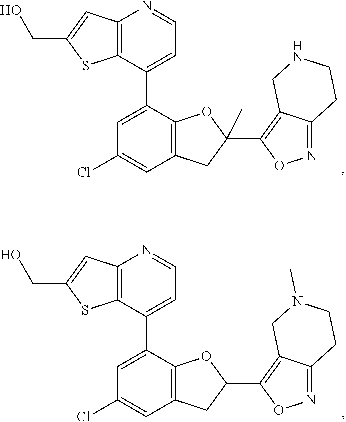 [SMILES: CC1(c2onc3c2CNCC3)Cc2cc(Cl)cc(-c3ccnc4cc(CO)sc34)c2O1 ; CN1CCc2noc(C3Cc4cc(Cl)cc(-c5ccnc6cc(CO)sc56)c4O3)c2C1]

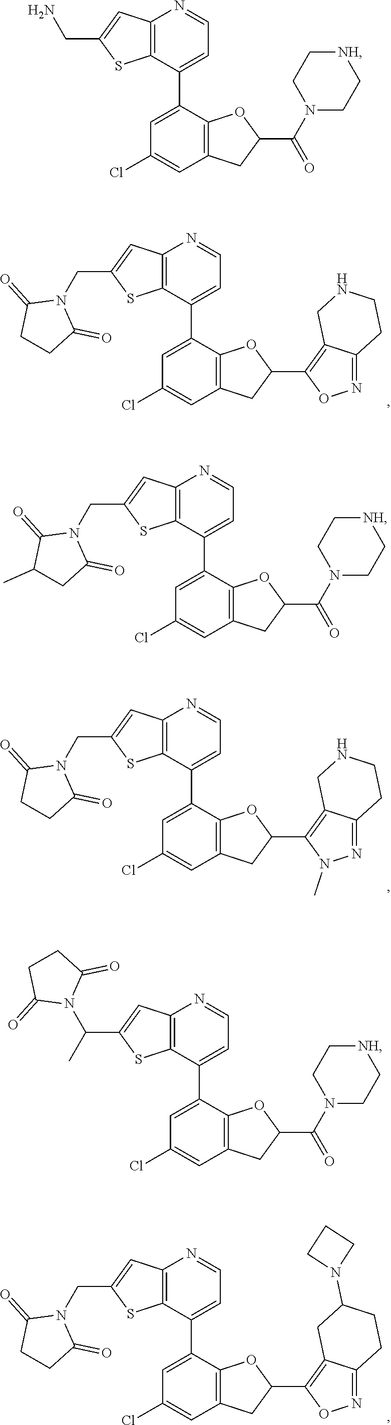 [SMILES: NCc1cc2nccc(-c3cc(Cl)cc4c3OC(C(=O)N3CCNCC3)C4)c2s1 ; O=C1CCC(=O)N1Cc1cc2nccc(-c3cc(Cl)cc4c3OC(c3onc5c3CNCC5)C4)c2s1 ; CC1CC(=O)N(Cc2cc3nccc(-c4cc(Cl)cc5c4OC(C(=O)N4CCNCC4)C5)c3s2)C1=O ; Cn1nc2c(c1C1Cc3cc(Cl)cc(-c4ccnc5cc(CN6C(=O)CCC6=O)sc45)c3O1)CNCC2 ; CC(c1cc2nccc(-c3cc(Cl)cc4c3OC(C(=O)N3CCNCC3)C4)c2s1)N1C(=O)CCC1=O ; O=C1CCC(=O)N1Cc1cc2nccc(-c3cc(Cl)cc4c3OC(c3onc5c3CC(N3CCC3)CC5)C4)c2s1]

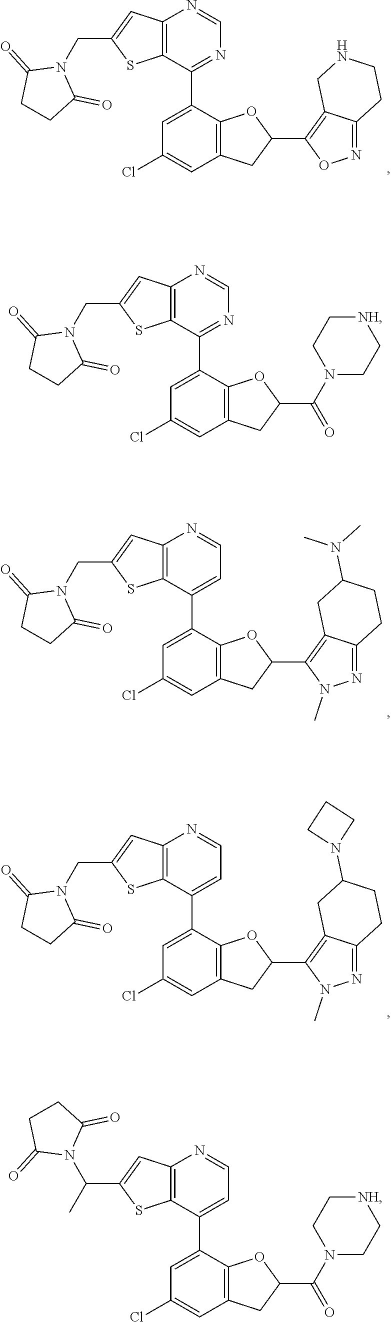 [SMILES: O=C1CCC(=O)N1Cc1cc2ncnc(-c3cc(Cl)cc4c3OC(c3onc5c3CNCC5)C4)c2s1 ; O=C(C1Cc2cc(Cl)cc(-c3ncnc4cc(CN5C(=O)CCC5=O)sc34)c2O1)N1CCNCC1 ; CN(C)C1CCc2nn(C)c(C3Cc4cc(Cl)cc(-c5ccnc6cc(CN7C(=O)CCC7=O)sc56)c4O3)c2C1 ; Cn1nc2c(c1C1Cc3cc(Cl)cc(-c4ccnc5cc(CN6C(=O)CCC6=O)sc45)c3O1)CC(N1CCC1)CC2 ; CC(c1cc2nccc(-c3cc(Cl)cc4c3OC(C(=O)N3CCNCC3)C4)c2s1)N1C(=O)CCC1=O]

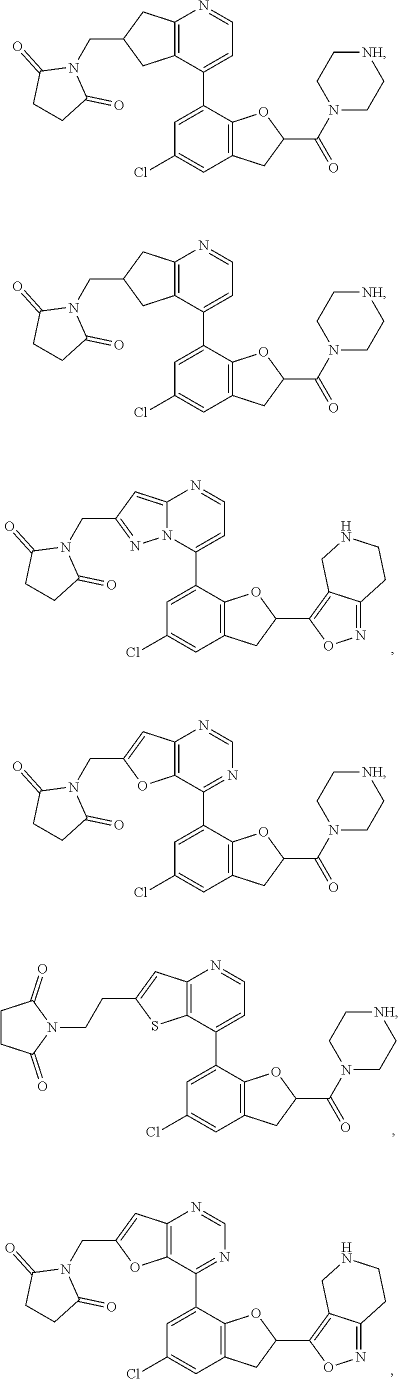 [SMILES: O=C(C1Cc2cc(Cl)cc(-c3ccnc4c3CC(CN3C(=O)CCC3=O)C4)c2O1)N1CCNCC1 ; O=C(C1Cc2cc(Cl)cc(-c3ccnc4c3CC(CN3C(=O)CCC3=O)C4)c2O1)N1CCNCC1 ; O=C1CCC(=O)N1Cc1cc2nccc(-c3cc(Cl)cc4c3OC(c3onc5c3CNCC5)C4)n2n1 ; O=C(C1Cc2cc(Cl)cc(-c3ncnc4cc(CN5C(=O)CCC5=O)oc34)c2O1)N1CCNCC1 ; O=C(C1Cc2cc(Cl)cc(-c3ccnc4cc(CCN5C(=O)CCC5=O)sc34)c2O1)N1CCNCC1 ; O=C1CCC(=O)N1Cc1cc2ncnc(-c3cc(Cl)cc4c3OC(c3onc5c3CNCC5)C4)c2o1]

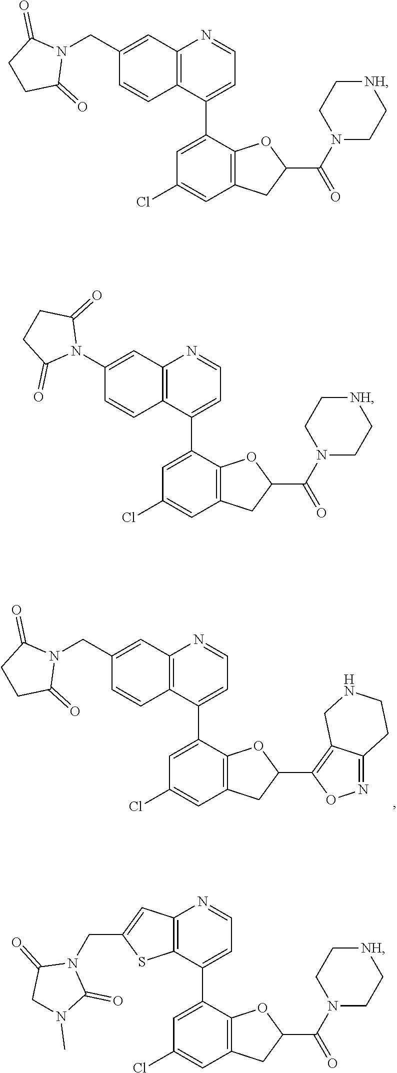 [SMILES: O=C(C1Cc2cc(Cl)cc(-c3ccnc4cc(CN5C(=O)CCC5=O)ccc34)c2O1)N1CCNCC1 ; O=C(C1Cc2cc(Cl)cc(-c3ccnc4cc(N5C(=O)CCC5=O)ccc34)c2O1)N1CCNCC1 ; O=C1CCC(=O)N1Cc1ccc2c(-c3cc(Cl)cc4c3OC(c3onc5c3CNCC5)C4)ccnc2c1 ; CN1CC(=O)N(Cc2cc3nccc(-c4cc(Cl)cc5c4OC(C(=O)N4CCNCC4)C5)c3s2)C1=O]

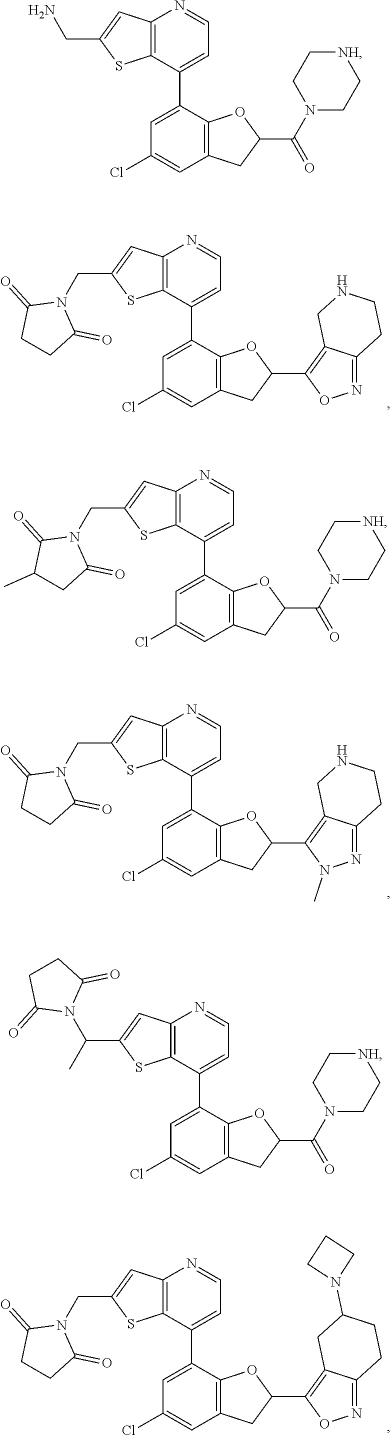 [SMILES: NCc1cc2nccc(-c3cc(Cl)cc4c3OC(C(=O)N3CCNCC3)C4)c2s1 ; O=C1CCC(=O)N1Cc1cc2nccc(-c3cc(Cl)cc4c3OC(c3onc5c3CNCC5)C4)c2s1 ; CC1CC(=O)N(Cc2cc3nccc(-c4cc(Cl)cc5c4OC(C(=O)N4CCNCC4)C5)c3s2)C1=O ; Cn1nc2c(c1C1Cc3cc(Cl)cc(-c4ccnc5cc(CN6C(=O)CCC6=O)sc45)c3O1)CNCC2 ; CC(c1cc2nccc(-c3cc(Cl)cc4c3OC(C(=O)N3CCNCC3)C4)c2s1)N1C(=O)CCC1=O ; O=C1CCC(=O)N1Cc1cc2nccc(-c3cc(Cl)cc4c3OC(c3onc5c3CC(N3CCC3)CC5)C4)c2s1]

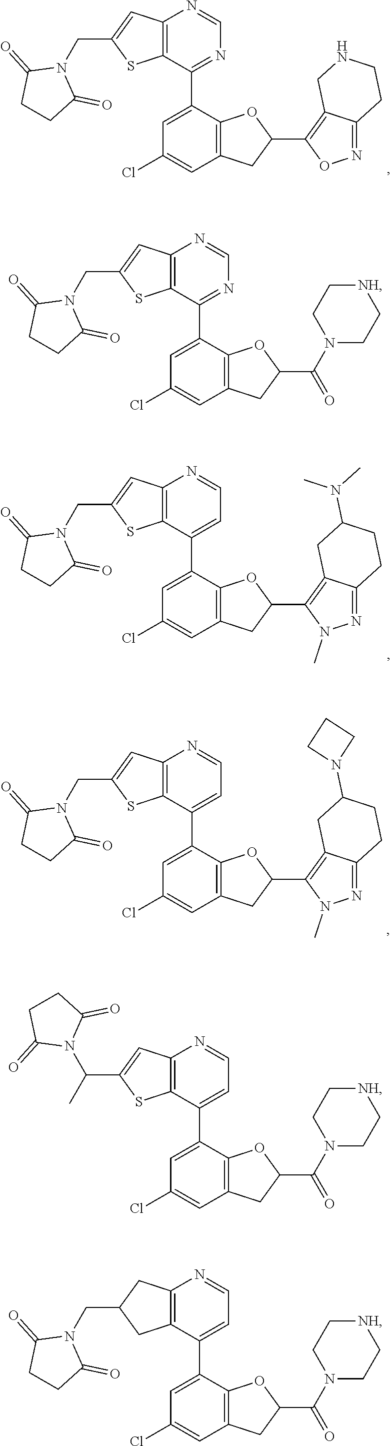 [SMILES: O=C1CCC(=O)N1Cc1cc2ncnc(-c3cc(Cl)cc4c3OC(c3onc5c3CNCC5)C4)c2s1 ; O=C(C1Cc2cc(Cl)cc(-c3ncnc4cc(CN5C(=O)CCC5=O)sc34)c2O1)N1CCNCC1 ; CN(C)C1CCc2nn(C)c(C3Cc4cc(Cl)cc(-c5ccnc6cc(CN7C(=O)CCC7=O)sc56)c4O3)c2C1 ; Cn1nc2c(c1C1Cc3cc(Cl)cc(-c4ccnc5cc(CN6C(=O)CCC6=O)sc45)c3O1)CC(N1CCC1)CC2 ; CC(c1cc2nccc(-c3cc(Cl)cc4c3OC(C(=O)N3CCNCC3)C4)c2s1)N1C(=O)CCC1=O ; O=C(C1Cc2cc(Cl)cc(-c3ccnc4c3CC(CN3C(=O)CCC3=O)C4)c2O1)N1CCNCC1]

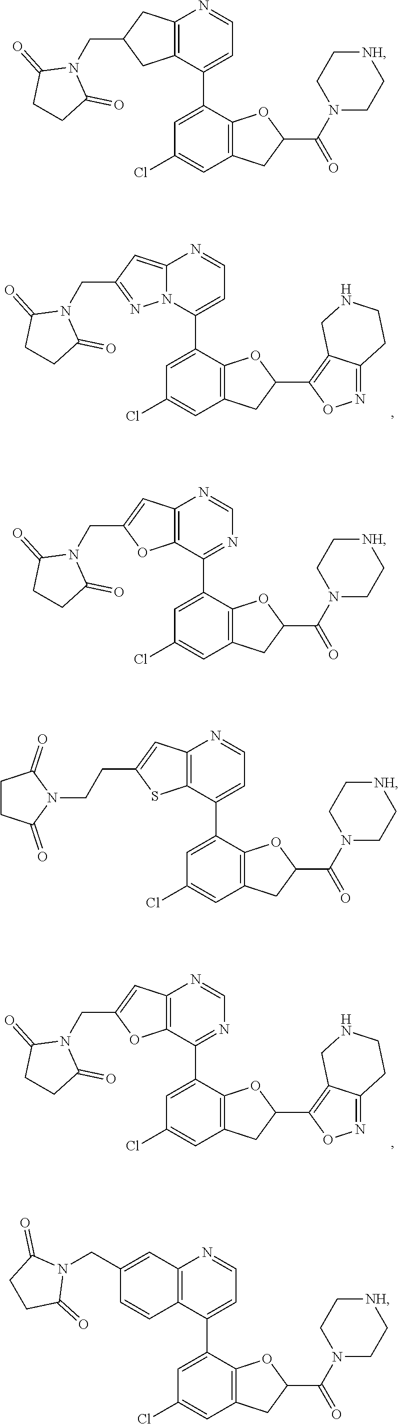 [SMILES: O=C(C1Cc2cc(Cl)cc(-c3ccnc4c3CC(CN3C(=O)CCC3=O)C4)c2O1)N1CCNCC1 ; O=C1CCC(=O)N1Cc1cc2nccc(-c3cc(Cl)cc4c3OC(c3onc5c3CNCC5)C4)n2n1 ; O=C(C1Cc2cc(Cl)cc(-c3ncnc4cc(CN5C(=O)CCC5=O)oc34)c2O1)N1CCNCC1 ; O=C(C1Cc2cc(Cl)cc(-c3ccnc4cc(CCN5C(=O)CCC5=O)sc34)c2O1)N1CCNCC1 ; O=C1CCC(=O)N1Cc1cc2ncnc(-c3cc(Cl)cc4c3OC(c3onc5c3CNCC5)C4)c2o1 ; O=C(C1Cc2cc(Cl)cc(-c3ccnc4cc(CN5C(=O)CCC5=O)ccc34)c2O1)N1CCNCC1]

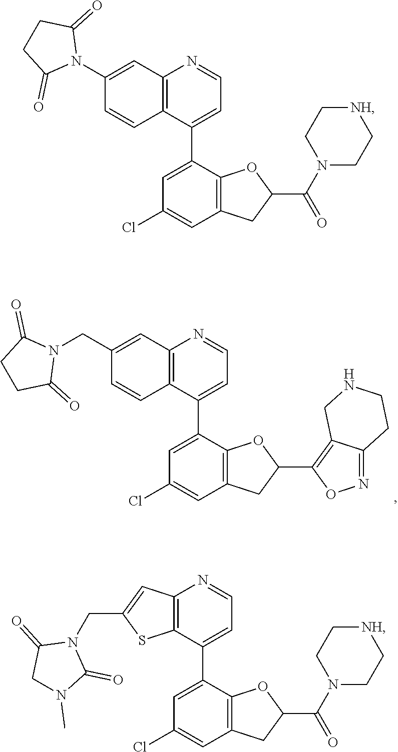 [SMILES: O=C(C1Cc2cc(Cl)cc(-c3ccnc4cc(N5C(=O)CCC5=O)ccc34)c2O1)N1CCNCC1 ; O=C1CCC(=O)N1Cc1ccc2c(-c3cc(Cl)cc4c3OC(c3onc5c3CNCC5)C4)ccnc2c1 ; CN1CC(=O)N(Cc2cc3nccc(-c4cc(Cl)cc5c4OC(C(=O)N4CCNCC4)C5)c3s2)C1=O]

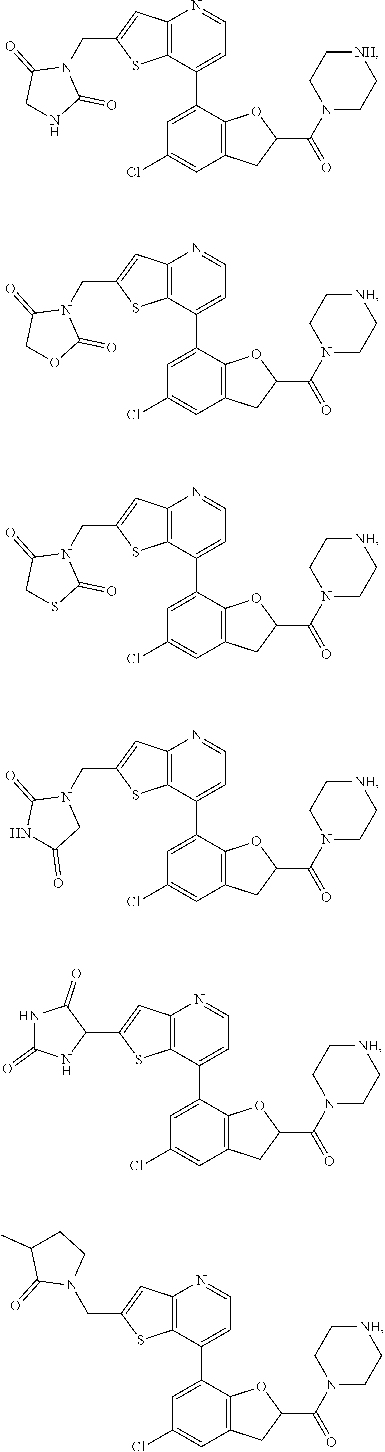 [SMILES: O=C(C1Cc2cc(Cl)cc(-c3ccnc4cc(CN5C(=O)CNC5=O)sc34)c2O1)N1CCNCC1 ; O=C(C1Cc2cc(Cl)cc(-c3ccnc4cc(CN5C(=O)COC5=O)sc34)c2O1)N1CCNCC1 ; O=C(C1Cc2cc(Cl)cc(-c3ccnc4cc(CN5C(=O)CSC5=O)sc34)c2O1)N1CCNCC1 ; O=C1CN(Cc2cc3nccc(-c4cc(Cl)cc5c4OC(C(=O)N4CCNCC4)C5)c3s2)C(=O)N1 ; O=C1NC(=O)C(c2cc3nccc(-c4cc(Cl)cc5c4OC(C(=O)N4CCNCC4)C5)c3s2)N1 ; CC1CCN(Cc2cc3nccc(-c4cc(Cl)cc5c4OC(C(=O)N4CCNCC4)C5)c3s2)C1=O]

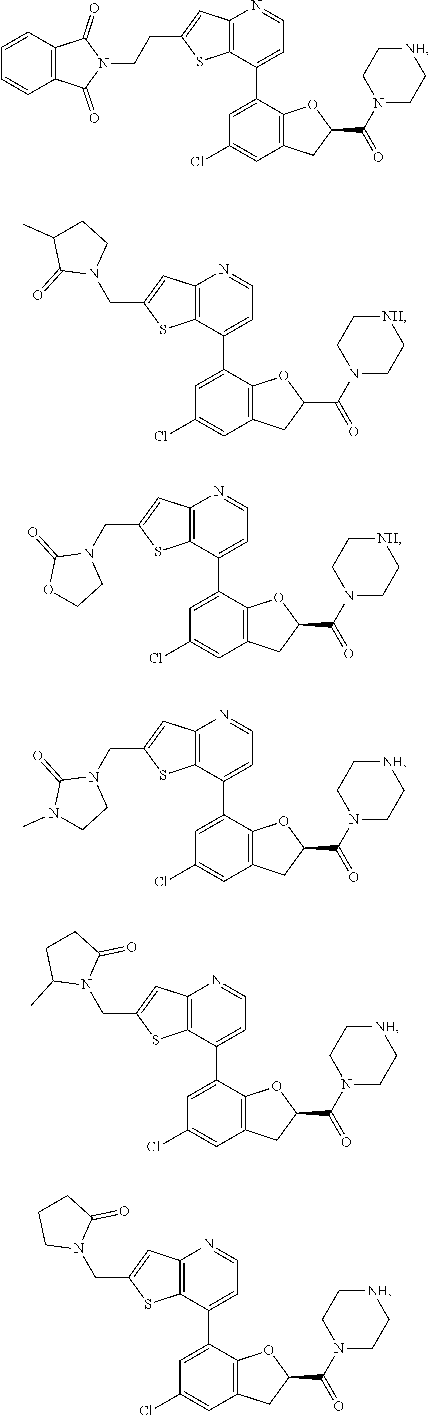 [SMILES: O=C([C@H]1Cc2cc(Cl)cc(-c3ccnc4cc(CCN5C(=O)c6ccccc6C5=O)sc34)c2O1)N1CCNCC1 ; CC1CCN(Cc2cc3nccc(-c4cc(Cl)cc5c4OC(C(=O)N4CCNCC4)C5)c3s2)C1=O ; O=C1OCCN1Cc1cc2nccc(-c3cc(Cl)cc4c3O[C@@H](C(=O)N3CCNCC3)C4)c2s1 ; CN1CCN(Cc2cc3nccc(-c4cc(Cl)cc5c4O[C@@H](C(=O)N4CCNCC4)C5)c3s2)C1=O ; CC1CCC(=O)N1Cc1cc2nccc(-c3cc(Cl)cc4c3O[C@@H](C(=O)N3CCNCC3)C4)c2s1 ; O=C1CCCN1Cc1cc2nccc(-c3cc(Cl)cc4c3O[C@@H](C(=O)N3CCNCC3)C4)c2s1]

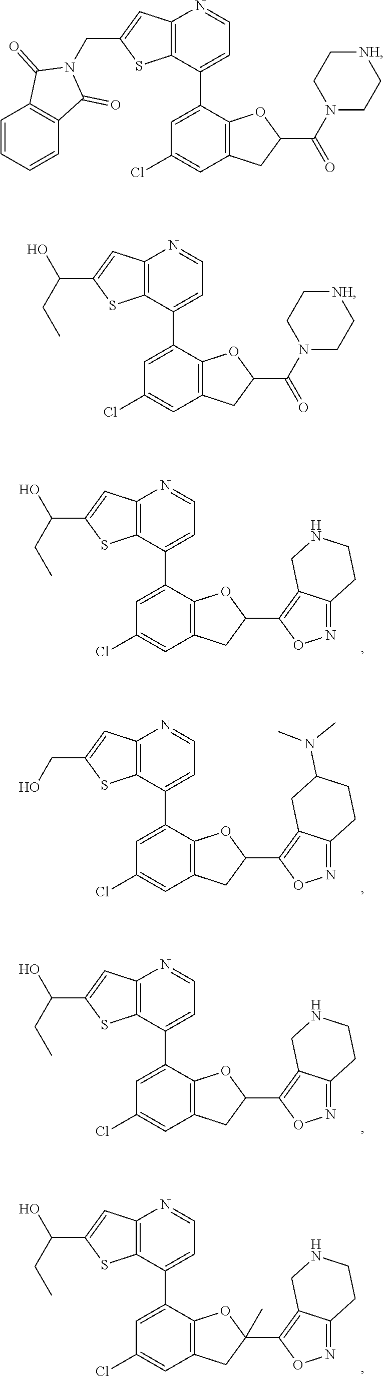 [SMILES: O=C(C1Cc2cc(Cl)cc(-c3ccnc4cc(CN5C(=O)c6ccccc6C5=O)sc34)c2O1)N1CCNCC1 ; CCC(O)c1cc2nccc(-c3cc(Cl)cc4c3OC(C(=O)N3CCNCC3)C4)c2s1 ; CCC(O)c1cc2nccc(-c3cc(Cl)cc4c3OC(c3onc5c3CNCC5)C4)c2s1 ; CN(C)C1CCc2noc(C3Cc4cc(Cl)cc(-c5ccnc6cc(CO)sc56)c4O3)c2C1 ; CCC(O)c1cc2nccc(-c3cc(Cl)cc4c3OC(c3onc5c3CNCC5)C4)c2s1 ; CCC(O)c1cc2nccc(-c3cc(Cl)cc4c3OC(C)(c3onc5c3CNCC5)C4)c2s1]

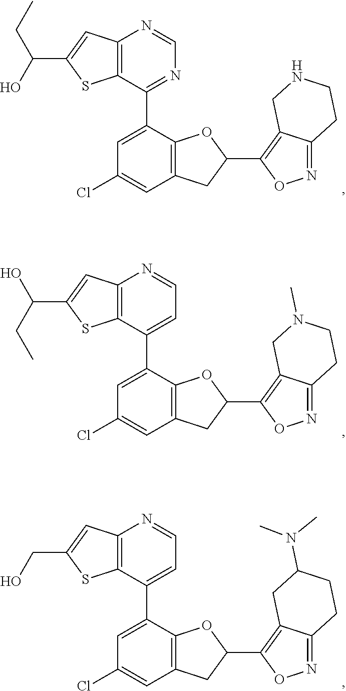 [SMILES: CCC(O)c1cc2ncnc(-c3cc(Cl)cc4c3OC(c3onc5c3CNCC5)C4)c2s1 ; CCC(O)c1cc2nccc(-c3cc(Cl)cc4c3OC(c3onc5c3CN(C)CC5)C4)c2s1 ; CN(C)C1CCc2noc(C3Cc4cc(Cl)cc(-c5ccnc6cc(CO)sc56)c4O3)c2C1]

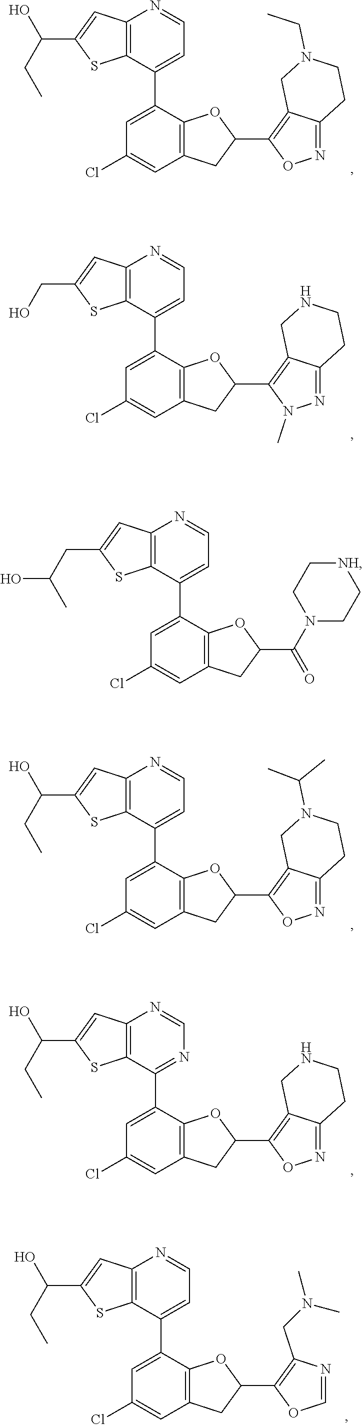 [SMILES: CCC(O)c1cc2nccc(-c3cc(Cl)cc4c3OC(c3onc5c3CN(CC)CC5)C4)c2s1 ; Cn1nc2c(c1C1Cc3cc(Cl)cc(-c4ccnc5cc(CO)sc45)c3O1)CNCC2 ; CC(O)Cc1cc2nccc(-c3cc(Cl)cc4c3OC(C(=O)N3CCNCC3)C4)c2s1 ; CCC(O)c1cc2nccc(-c3cc(Cl)cc4c3OC(c3onc5c3CN(C(C)C)CC5)C4)c2s1 ; CCC(O)c1cc2ncnc(-c3cc(Cl)cc4c3OC(c3onc5c3CNCC5)C4)c2s1 ; CCC(O)c1cc2nccc(-c3cc(Cl)cc4c3OC(c3ocnc3CN(C)C)C4)c2s1]

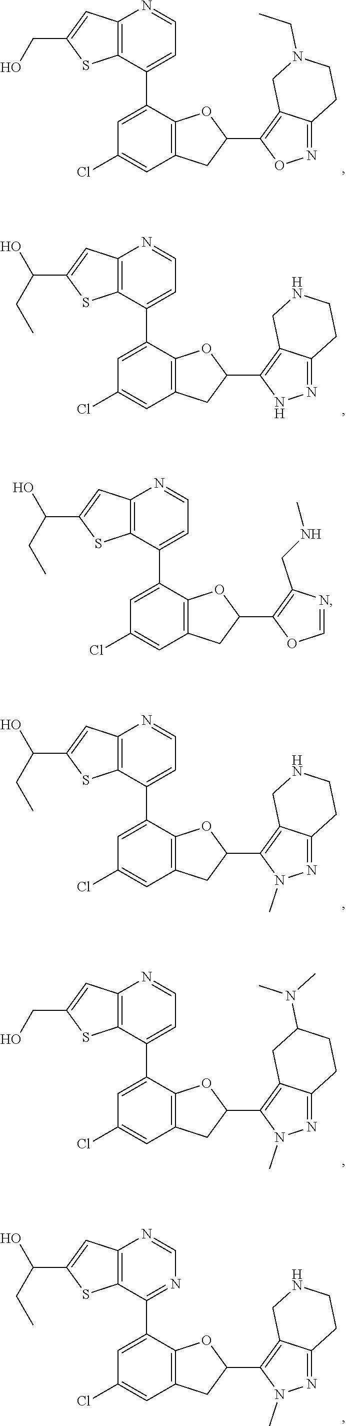 [SMILES: CCN1CCc2noc(C3Cc4cc(Cl)cc(-c5ccnc6cc(CO)sc56)c4O3)c2C1 ; CCC(O)c1cc2nccc(-c3cc(Cl)cc4c3OC(c3[nH]nc5c3CNCC5)C4)c2s1 ; CCC(O)c1cc2nccc(-c3cc(Cl)cc4c3OC(c3ocnc3CNC)C4)c2s1 ; CCC(O)c1cc2nccc(-c3cc(Cl)cc4c3OC(c3c5c(nn3C)CCNC5)C4)c2s1 ; CN(C)C1CCc2nn(C)c(C3Cc4cc(Cl)cc(-c5ccnc6cc(CO)sc56)c4O3)c2C1 ; CCC(O)c1cc2ncnc(-c3cc(Cl)cc4c3OC(c3c5c(nn3C)CCNC5)C4)c2s1]

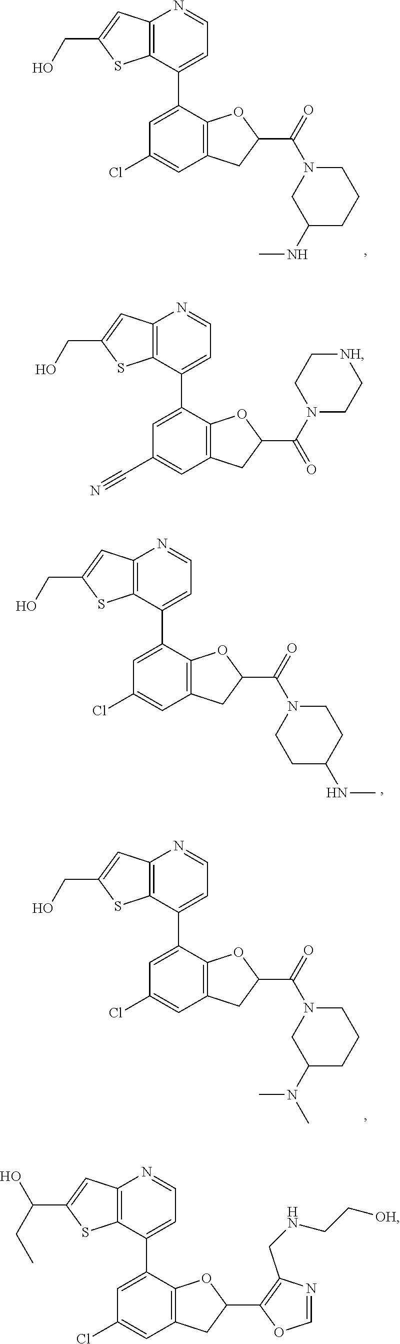 [SMILES: CNC1CCCN(C(=O)C2Cc3cc(Cl)cc(-c4ccnc5cc(CO)sc45)c3O2)C1 ; N#Cc1cc2c(c(-c3ccnc4cc(CO)sc34)c1)OC(C(=O)N1CCNCC1)C2 ; CNC1CCN(C(=O)C2Cc3cc(Cl)cc(-c4ccnc5cc(CO)sc45)c3O2)CC1 ; CN(C)C1CCCN(C(=O)C2Cc3cc(Cl)cc(-c4ccnc5cc(CO)sc45)c3O2)C1 ; CCC(O)c1cc2nccc(-c3cc(Cl)cc4c3OC(c3ocnc3CNCCO)C4)c2s1]

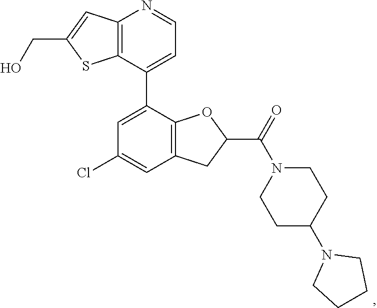 [SMILES: O=C(C1Cc2cc(Cl)cc(-c3ccnc4cc(CO)sc34)c2O1)N1CCC(N2CCCC2)CC1]

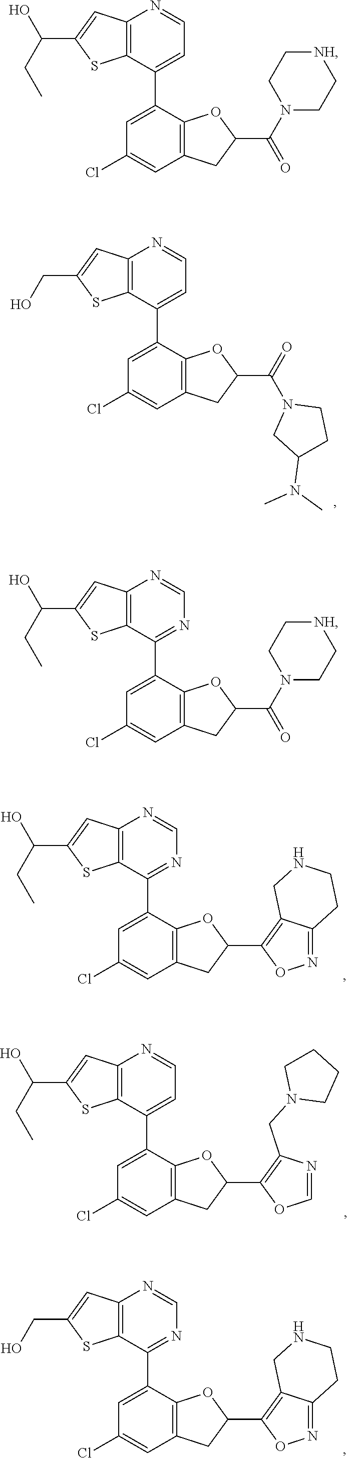 [SMILES: CCC(O)c1cc2nccc(-c3cc(Cl)cc4c3OC(C(=O)N3CCNCC3)C4)c2s1 ; CN(C)C1CCN(C(=O)C2Cc3cc(Cl)cc(-c4ccnc5cc(CO)sc45)c3O2)C1 ; CCC(O)c1cc2ncnc(-c3cc(Cl)cc4c3OC(C(=O)N3CCNCC3)C4)c2s1 ; CCC(O)c1cc2ncnc(-c3cc(Cl)cc4c3OC(c3onc5c3CNCC5)C4)c2s1 ; CCC(O)c1cc2nccc(-c3cc(Cl)cc4c3OC(c3ocnc3CN3CCCC3)C4)c2s1 ; OCc1cc2ncnc(-c3cc(Cl)cc4c3OC(c3onc5c3CNCC5)C4)c2s1]

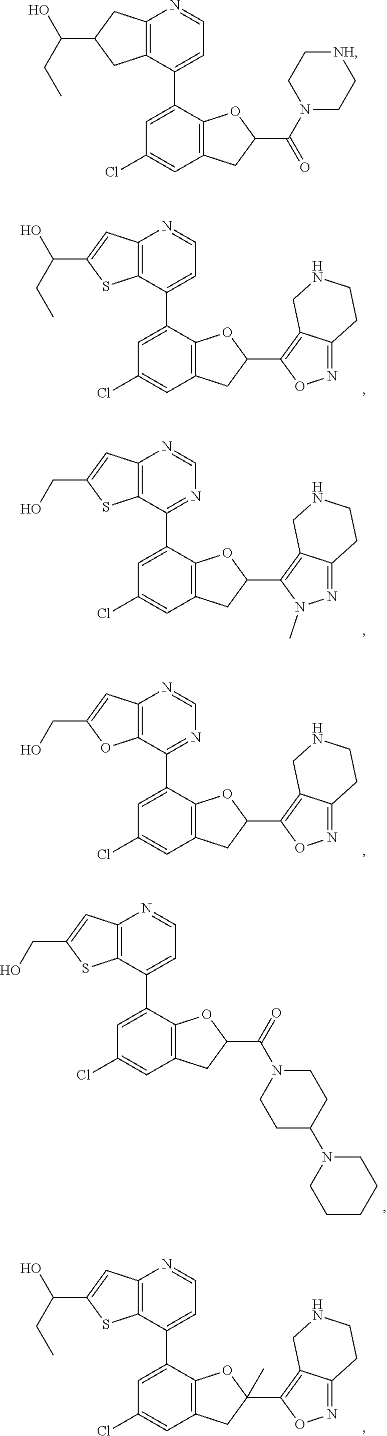 [SMILES: CCC(O)C1Cc2nccc(-c3cc(Cl)cc4c3OC(C(=O)N3CCNCC3)C4)c2C1 ; CCC(O)c1cc2nccc(-c3cc(Cl)cc4c3OC(c3onc5c3CNCC5)C4)c2s1 ; Cn1nc2c(c1C1Cc3cc(Cl)cc(-c4ncnc5cc(CO)sc45)c3O1)CNCC2 ; OCc1cc2ncnc(-c3cc(Cl)cc4c3OC(c3onc5c3CNCC5)C4)c2o1 ; O=C(C1Cc2cc(Cl)cc(-c3ccnc4cc(CO)sc34)c2O1)N1CCC(N2CCCCC2)CC1 ; CCC(O)c1cc2nccc(-c3cc(Cl)cc4c3OC(C)(c3onc5c3CNCC5)C4)c2s1]

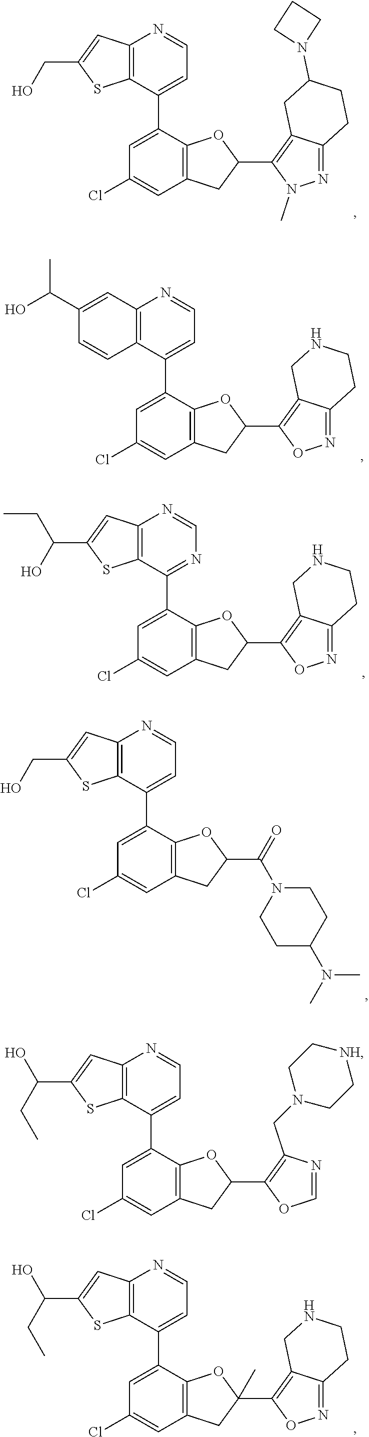

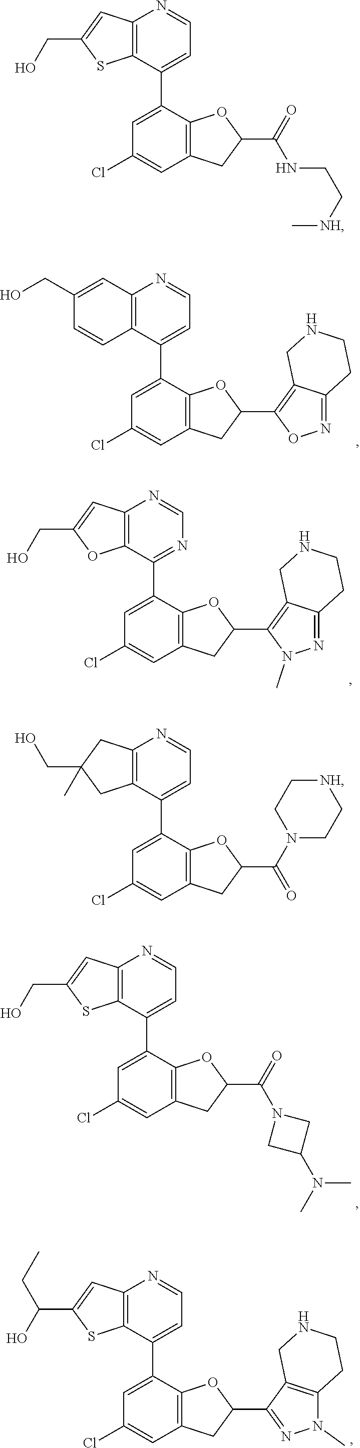 [SMILES: CNCCNC(=O)C1Cc2cc(Cl)cc(-c3ccnc4cc(CO)sc34)c2O1 ; OCc1ccc2c(-c3cc(Cl)cc4c3OC(c3onc5c3CNCC5)C4)ccnc2c1 ; Cn1nc2c(c1C1Cc3cc(Cl)cc(-c4ncnc5cc(CO)oc45)c3O1)CNCC2 ; CC1(CO)Cc2nccc(-c3cc(Cl)cc4c3OC(C(=O)N3CCNCC3)C4)c2C1 ; CN(C)C1CN(C(=O)C2Cc3cc(Cl)cc(-c4ccnc5cc(CO)sc45)c3O2)C1 ; CCC(O)c1cc2nccc(-c3cc(Cl)cc4c3OC(c3nn(C)c5c3CNCC5)C4)c2s1]

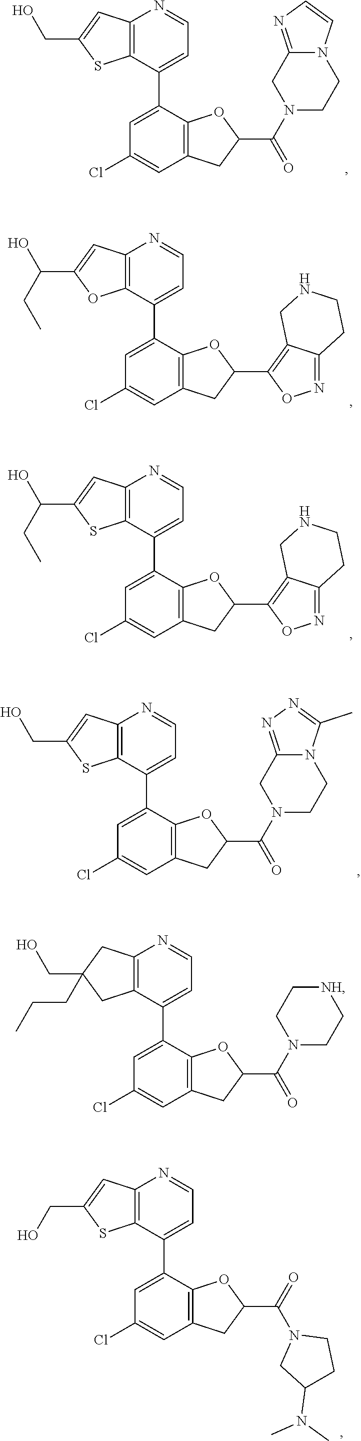

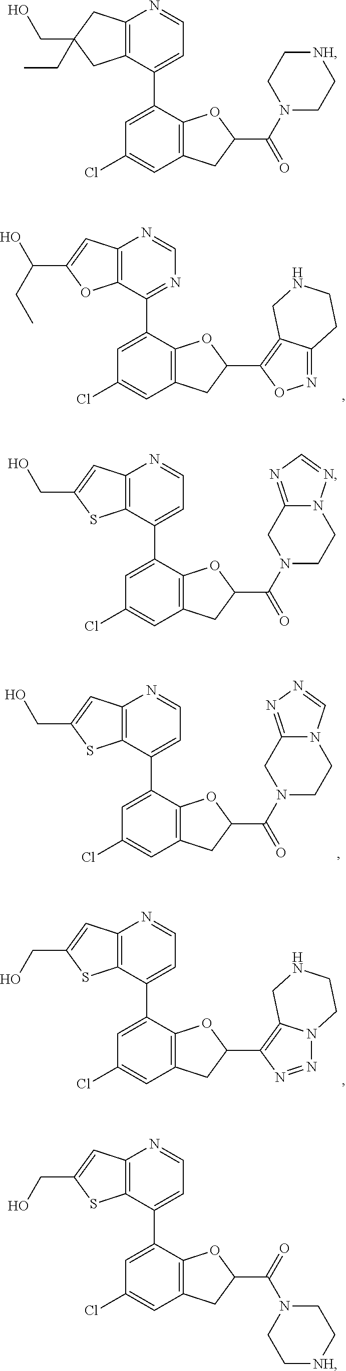 [SMILES: CCC1(CO)Cc2nccc(-c3cc(Cl)cc4c3OC(C(=O)N3CCNCC3)C4)c2C1 ; CCC(O)c1cc2ncnc(-c3cc(Cl)cc4c3OC(c3onc5c3CNCC5)C4)c2o1 ; O=C(C1Cc2cc(Cl)cc(-c3ccnc4cc(CO)sc34)c2O1)N1CCn2ncnc2C1 ; O=C(C1Cc2cc(Cl)cc(-c3ccnc4cc(CO)sc34)c2O1)N1CCn2cnnc2C1 ; OCc1cc2nccc(-c3cc(Cl)cc4c3OC(c3nnn5c3CNCC5)C4)c2s1 ; O=C(C1Cc2cc(Cl)cc(-c3ccnc4cc(CO)sc34)c2O1)N1CCNCC1]

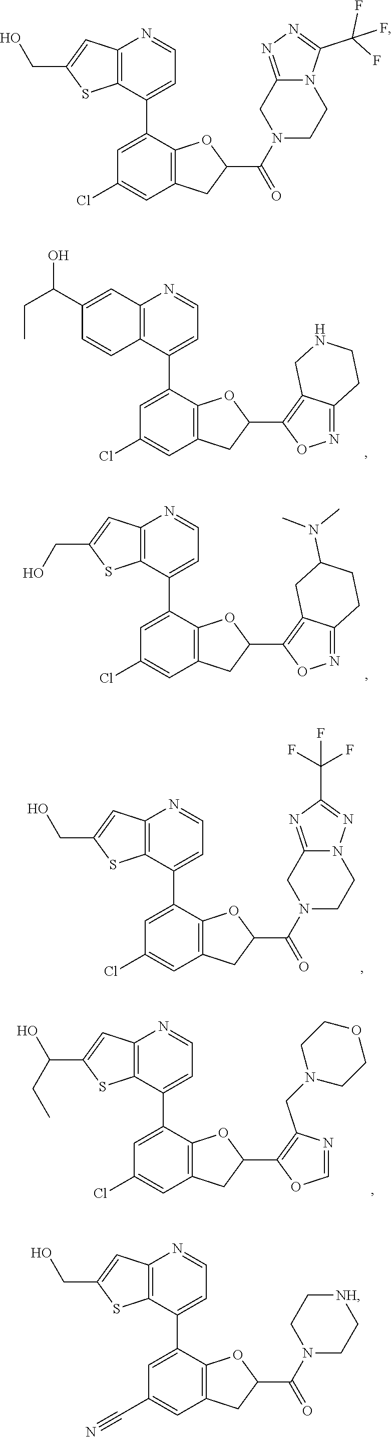 [SMILES: O=C(C1Cc2cc(Cl)cc(-c3ccnc4cc(CO)sc34)c2O1)N1CCn2c(nnc2C(F)(F)F)C1 ; CCC(O)c1ccc2c(-c3cc(Cl)cc4c3OC(c3onc5c3CNCC5)C4)ccnc2c1 ; CN(C)C1CCc2noc(C3Cc4cc(Cl)cc(-c5ccnc6cc(CO)sc56)c4O3)c2C1 ; O=C(C1Cc2cc(Cl)cc(-c3ccnc4cc(CO)sc34)c2O1)N1CCn2nc(C(F)(F)F)nc2C1 ; CCC(O)c1cc2nccc(-c3cc(Cl)cc4c3OC(c3ocnc3CN3CCOCC3)C4)c2s1 ; N#Cc1cc2c(c(-c3ccnc4cc(CO)sc34)c1)OC(C(=O)N1CCNCC1)C2]

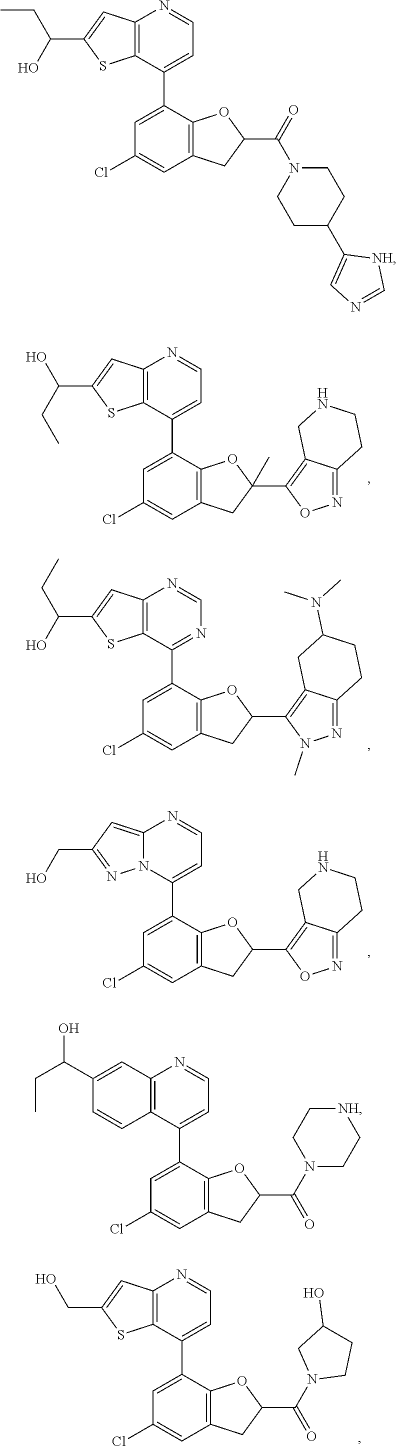

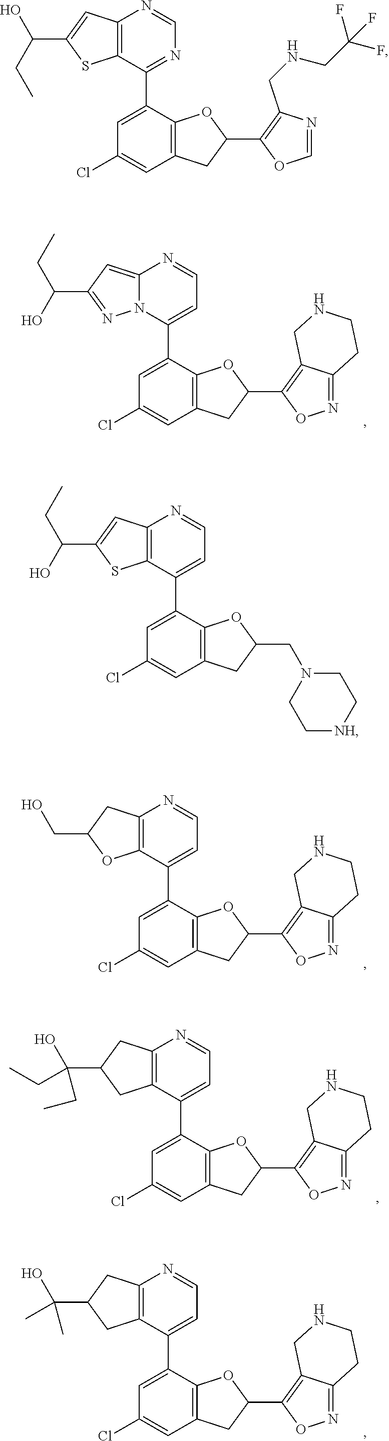 [SMILES: CCC(O)c1cc2ncnc(-c3cc(Cl)cc4c3OC(c3ocnc3CNCC(F)(F)F)C4)c2s1 ; CCC(O)c1cc2nccc(-c3cc(Cl)cc4c3OC(c3onc5c3CNCC5)C4)n2n1 ; CCC(O)c1cc2nccc(-c3cc(Cl)cc4c3OC(CN3CCNCC3)C4)c2s1 ; OCC1Cc2nccc(-c3cc(Cl)cc4c3OC(c3onc5c3CNCC5)C4)c2O1 ; CCC(O)(CC)C1Cc2nccc(-c3cc(Cl)cc4c3OC(c3onc5c3CNCC5)C4)c2C1 ; CC(C)(O)C1Cc2nccc(-c3cc(Cl)cc4c3OC(c3onc5c3CNCC5)C4)c2C1]

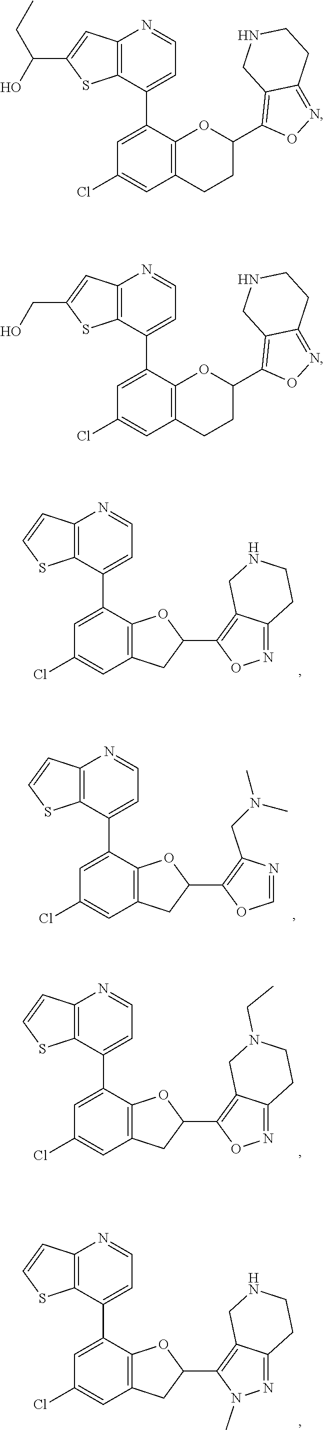 [SMILES: CCC(O)c1cc2nccc(-c3cc(Cl)cc4c3OC(c3onc5c3CNCC5)CC4)c2s1 ; OCc1cc2nccc(-c3cc(Cl)cc4c3OC(c3onc5c3CNCC5)CC4)c2s1 ; Clc1cc2c(c(-c3ccnc4ccsc34)c1)OC(c1onc3c1CNCC3)C2 ; CN(C)Cc1ncoc1C1Cc2cc(Cl)cc(-c3ccnc4ccsc34)c2O1 ; CCN1CCc2noc(C3Cc4cc(Cl)cc(-c5ccnc6ccsc56)c4O3)c2C1 ; Cn1nc2c(c1C1Cc3cc(Cl)cc(-c4ccnc5ccsc45)c3O1)CNCC2]

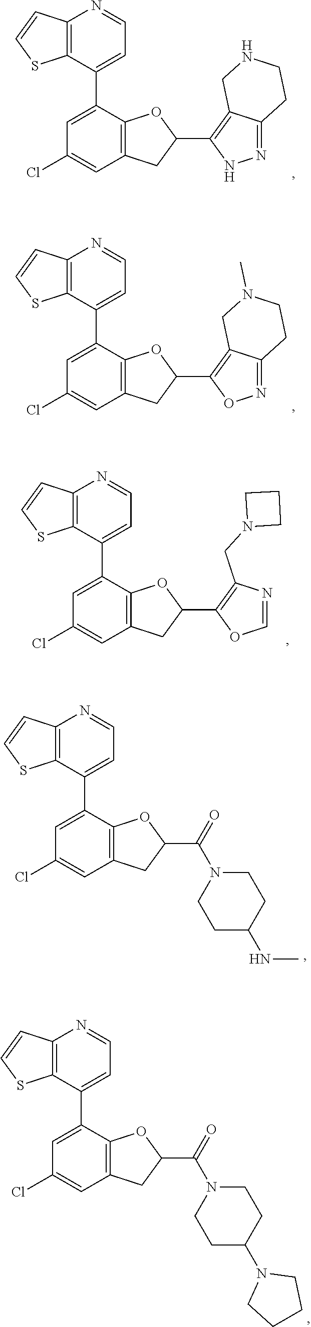 [SMILES: Clc1cc2c(c(-c3ccnc4ccsc34)c1)OC(c1[nH]nc3c1CNCC3)C2 ; CN1CCc2noc(C3Cc4cc(Cl)cc(-c5ccnc6ccsc56)c4O3)c2C1 ; Clc1cc2c(c(-c3ccnc4ccsc34)c1)OC(c1ocnc1CN1CCC1)C2 ; CNC1CCN(C(=O)C2Cc3cc(Cl)cc(-c4ccnc5ccsc45)c3O2)CC1 ; O=C(C1Cc2cc(Cl)cc(-c3ccnc4ccsc34)c2O1)N1CCC(N2CCCC2)CC1]

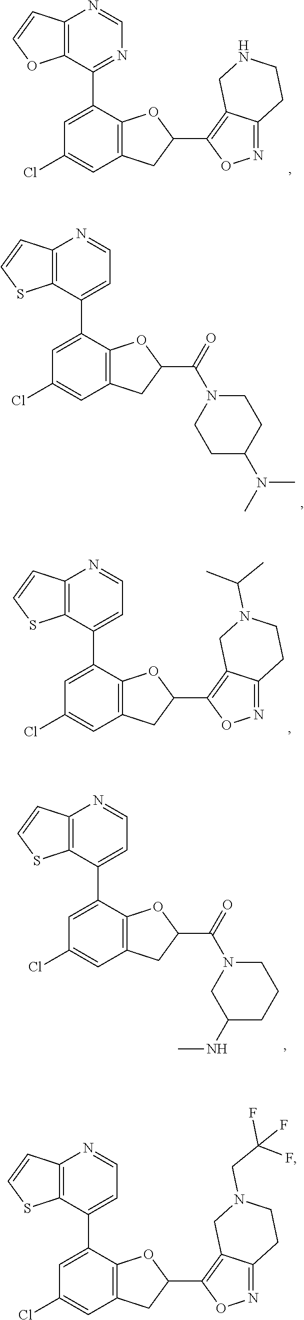 [SMILES: Clc1cc2c(c(-c3ncnc4ccoc34)c1)OC(c1onc3c1CNCC3)C2 ; CN(C)C1CCN(C(=O)C2Cc3cc(Cl)cc(-c4ccnc5ccsc45)c3O2)CC1 ; CC(C)N1CCc2noc(C3Cc4cc(Cl)cc(-c5ccnc6ccsc56)c4O3)c2C1 ; CNC1CCCN(C(=O)C2Cc3cc(Cl)cc(-c4ccnc5ccsc45)c3O2)C1 ; FC(F)(F)CN1CCc2noc(C3Cc4cc(Cl)cc(-c5ccnc6ccsc56)c4O3)c2C1]

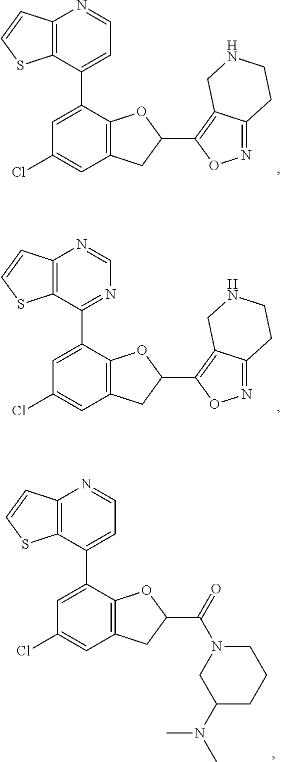 [SMILES: Clc1cc2c(c(-c3ccnc4ccsc34)c1)OC(c1onc3c1CNCC3)C2 ; Clc1cc2c(c(-c3ncnc4ccsc34)c1)OC(c1onc3c1CNCC3)C2 ; CN(C)C1CCCN(C(=O)C2Cc3cc(Cl)cc(-c4ccnc5ccsc45)c3O2)C1]

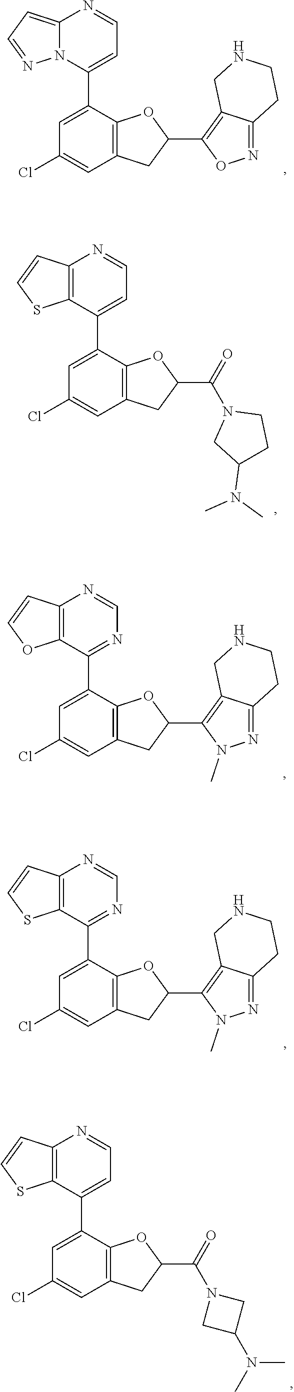 [SMILES: Clc1cc2c(c(-c3ccnc4ccnn34)c1)OC(c1onc3c1CNCC3)C2 ; CN(C)C1CCN(C(=O)C2Cc3cc(Cl)cc(-c4ccnc5ccsc45)c3O2)C1 ; Cn1nc2c(c1C1Cc3cc(Cl)cc(-c4ncnc5ccoc45)c3O1)CNCC2 ; Cn1nc2c(c1C1Cc3cc(Cl)cc(-c4ncnc5ccsc45)c3O1)CNCC2 ; CN(C)C1CN(C(=O)C2Cc3cc(Cl)cc(-c4ccnc5ccsc45)c3O2)C1]

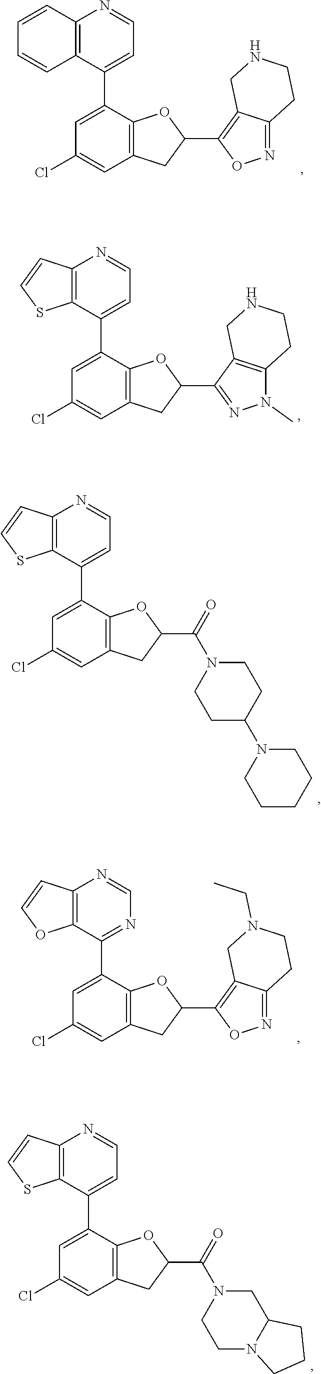 [SMILES: Clc1cc2c(c(-c3ccnc4ccccc34)c1)OC(c1onc3c1CNCC3)C2 ; Cn1nc(C2Cc3cc(Cl)cc(-c4ccnc5ccsc45)c3O2)c2c1CCNC2 ; O=C(C1Cc2cc(Cl)cc(-c3ccnc4ccsc34)c2O1)N1CCC(N2CCCCC2)CC1 ; CCN1CCc2noc(C3Cc4cc(Cl)cc(-c5ncnc6ccoc56)c4O3)c2C1 ; O=C(C1Cc2cc(Cl)cc(-c3ccnc4ccsc34)c2O1)N1CCN2CCCC2C1]

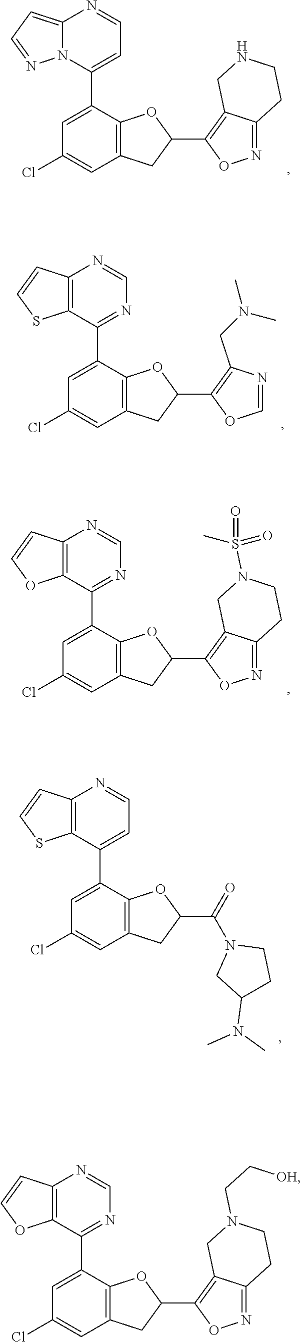 [SMILES: Clc1cc2c(c(-c3ccnc4ccnn34)c1)OC(c1onc3c1CNCC3)C2 ; CN(C)Cc1ncoc1C1Cc2cc(Cl)cc(-c3ncnc4ccsc34)c2O1 ; CS(=O)(=O)N1CCc2noc(C3Cc4cc(Cl)cc(-c5ncnc6ccoc56)c4O3)c2C1 ; CN(C)C1CCN(C(=O)C2Cc3cc(Cl)cc(-c4ccnc5ccsc45)c3O2)C1 ; OCCN1CCc2noc(C3Cc4cc(Cl)cc(-c5ncnc6ccoc56)c4O3)c2C1]

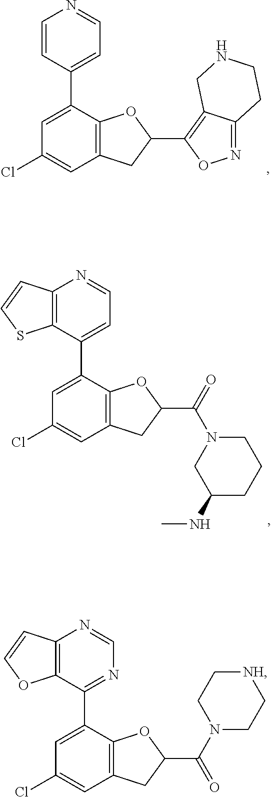 [SMILES: Clc1cc2c(c(-c3ccncc3)c1)OC(c1onc3c1CNCC3)C2 ; CN[C@@H]1CCCN(C(=O)C2Cc3cc(Cl)cc(-c4ccnc5ccsc45)c3O2)C1 ; O=C(C1Cc2cc(Cl)cc(-c3ncnc4ccoc34)c2O1)N1CCNCC1]

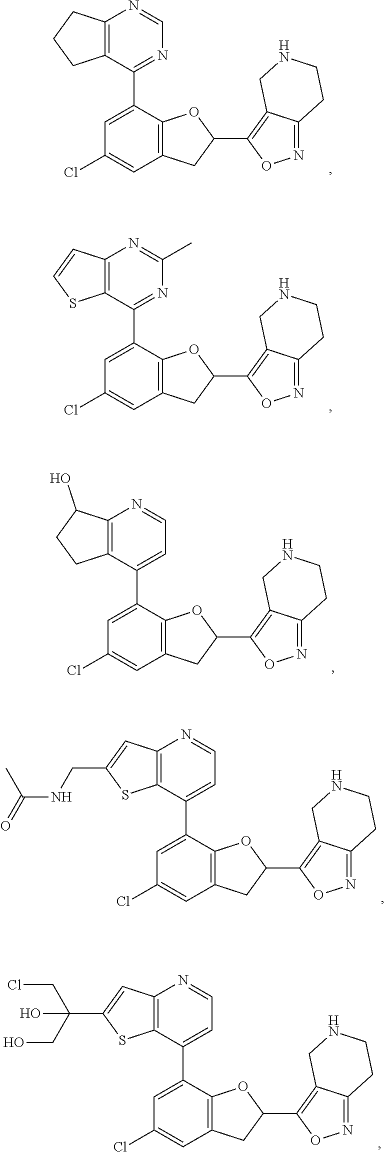 [SMILES: Clc1cc2c(c(-c3ncnc4c3CCC4)c1)OC(c1onc3c1CNCC3)C2 ; Cc1nc(-c2cc(Cl)cc3c2OC(c2onc4c2CNCC4)C3)c2sccc2n1 ; OC1CCc2c(-c3cc(Cl)cc4c3OC(c3onc5c3CNCC5)C4)ccnc21 ; CC(=O)NCc1cc2nccc(-c3cc(Cl)cc4c3OC(c3onc5c3CNCC5)C4)c2s1 ; OCC(O)(CCl)c1cc2nccc(-c3cc(Cl)cc4c3OC(c3onc5c3CNCC5)C4)c2s1]

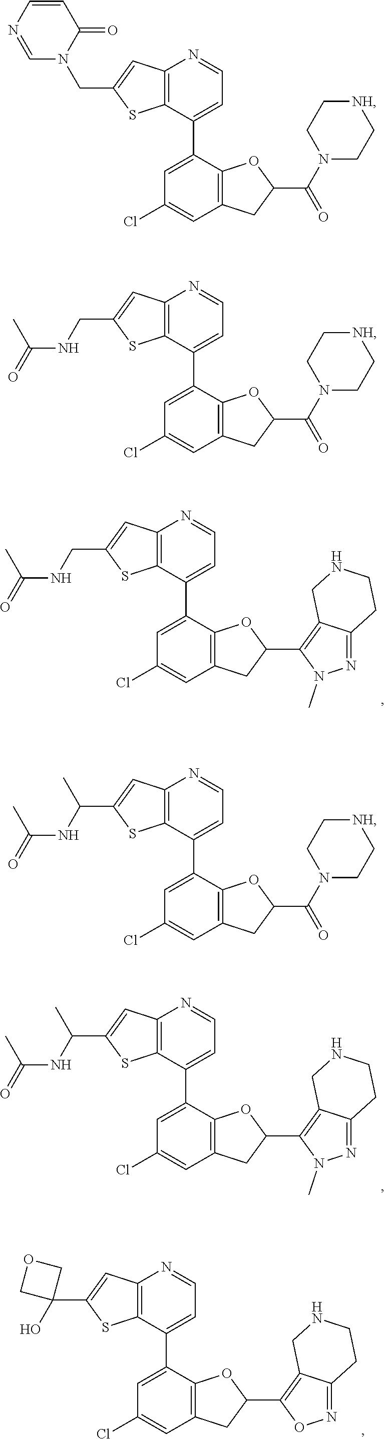 [SMILES: O=C(C1Cc2cc(Cl)cc(-c3ccnc4cc(Cn5cnccc5=O)sc34)c2O1)N1CCNCC1 ; CC(=O)NCc1cc2nccc(-c3cc(Cl)cc4c3OC(C(=O)N3CCNCC3)C4)c2s1 ; CC(=O)NCc1cc2nccc(-c3cc(Cl)cc4c3OC(c3c5c(nn3C)CCNC5)C4)c2s1 ; CC(=O)NC(C)c1cc2nccc(-c3cc(Cl)cc4c3OC(C(=O)N3CCNCC3)C4)c2s1 ; CC(=O)NC(C)c1cc2nccc(-c3cc(Cl)cc4c3OC(c3c5c(nn3C)CCNC5)C4)c2s1 ; OC1(c2cc3nccc(-c4cc(Cl)cc5c4OC(c4onc6c4CNCC6)C5)c3s2)COC1]

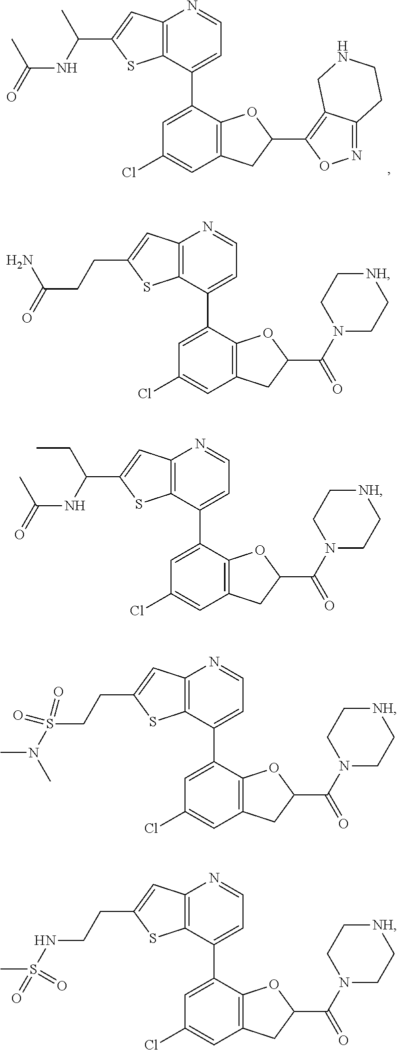 [SMILES: CC(=O)NC(C)c1cc2nccc(-c3cc(Cl)cc4c3OC(c3onc5c3CNCC5)C4)c2s1 ; NC(=O)CCc1cc2nccc(-c3cc(Cl)cc4c3OC(C(=O)N3CCNCC3)C4)c2s1 ; CCC(NC(C)=O)c1cc2nccc(-c3cc(Cl)cc4c3OC(C(=O)N3CCNCC3)C4)c2s1 ; CN(C)S(=O)(=O)CCc1cc2nccc(-c3cc(Cl)cc4c3OC(C(=O)N3CCNCC3)C4)c2s1 ; CS(=O)(=O)NCCc1cc2nccc(-c3cc(Cl)cc4c3OC(C(=O)N3CCNCC3)C4)c2s1]

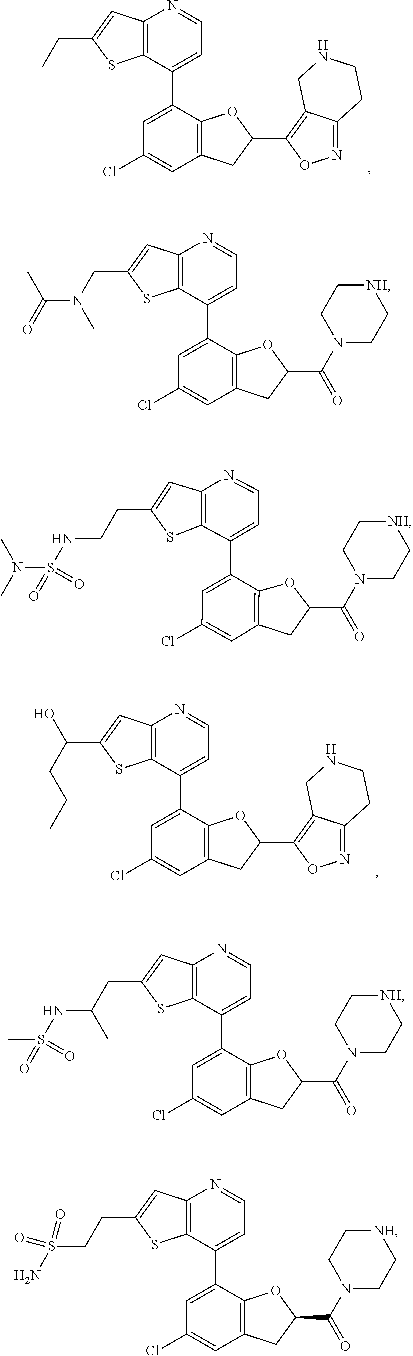 [SMILES: CCc1cc2nccc(-c3cc(Cl)cc4c3OC(c3onc5c3CNCC5)C4)c2s1 ; CC(=O)N(C)Cc1cc2nccc(-c3cc(Cl)cc4c3OC(C(=O)N3CCNCC3)C4)c2s1 ; CN(C)S(=O)(=O)NCCc1cc2nccc(-c3cc(Cl)cc4c3OC(C(=O)N3CCNCC3)C4)c2s1 ; CCCC(O)c1cc2nccc(-c3cc(Cl)cc4c3OC(c3onc5c3CNCC5)C4)c2s1 ; CC(Cc1cc2nccc(-c3cc(Cl)cc4c3OC(C(=O)N3CCNCC3)C4)c2s1)NS(C)(=O)=O ; NS(=O)(=O)CCc1cc2nccc(-c3cc(Cl)cc4c3O[C@@H](C(=O)N3CCNCC3)C4)c2s1]

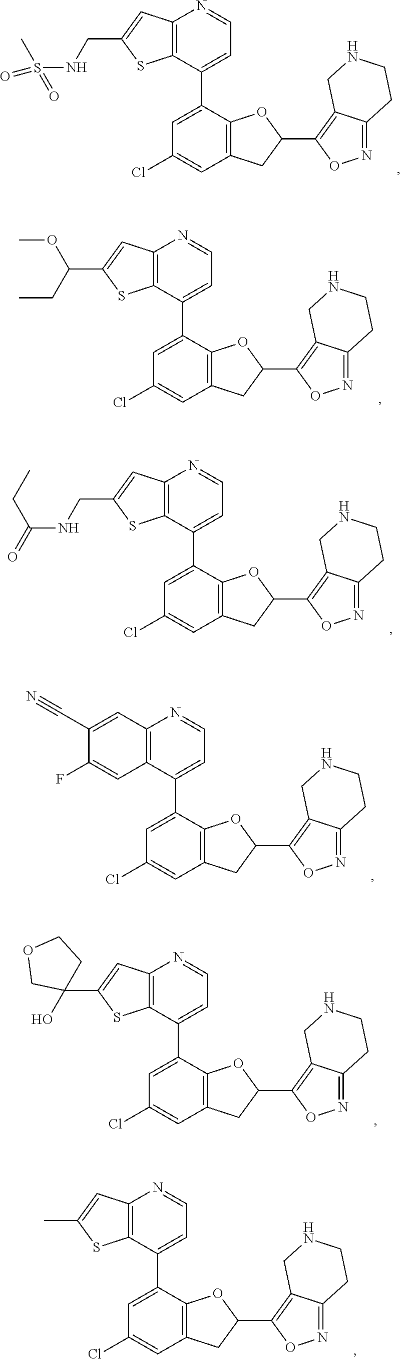 [SMILES: CS(=O)(=O)NCc1cc2nccc(-c3cc(Cl)cc4c3OC(c3onc5c3CNCC5)C4)c2s1 ; CCC(OC)c1cc2nccc(-c3cc(Cl)cc4c3OC(c3onc5c3CNCC5)C4)c2s1 ; CCC(=O)NCc1cc2nccc(-c3cc(Cl)cc4c3OC(c3onc5c3CNCC5)C4)c2s1 ; N#Cc1cc2nccc(-c3cc(Cl)cc4c3OC(c3onc5c3CNCC5)C4)c2cc1F ; OC1(c2cc3nccc(-c4cc(Cl)cc5c4OC(c4onc6c4CNCC6)C5)c3s2)CCOC1 ; Cc1cc2nccc(-c3cc(Cl)cc4c3OC(c3onc5c3CNCC5)C4)c2s1]

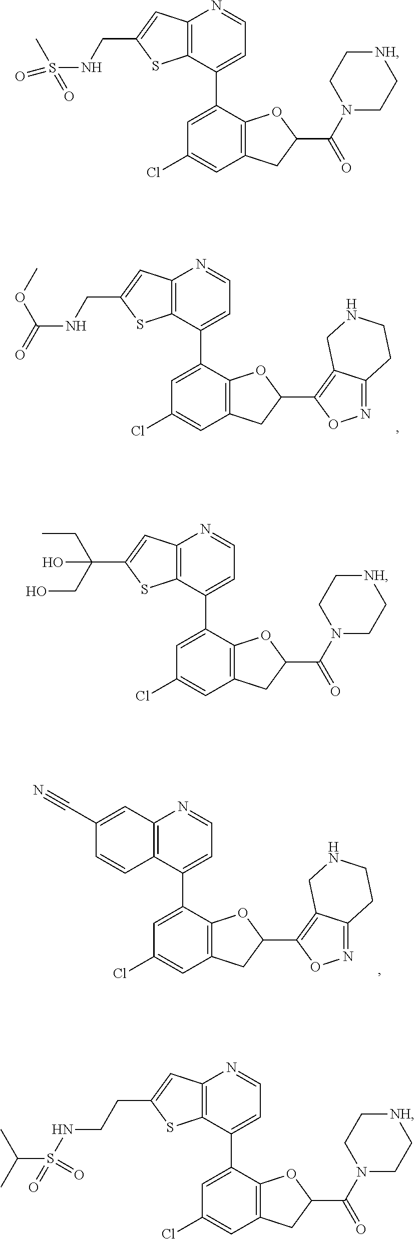 [SMILES: CS(=O)(=O)NCc1cc2nccc(-c3cc(Cl)cc4c3OC(C(=O)N3CCNCC3)C4)c2s1 ; COC(=O)NCc1cc2nccc(-c3cc(Cl)cc4c3OC(c3onc5c3CNCC5)C4)c2s1 ; CCC(O)(CO)c1cc2nccc(-c3cc(Cl)cc4c3OC(C(=O)N3CCNCC3)C4)c2s1 ; N#Cc1ccc2c(-c3cc(Cl)cc4c3OC(c3onc5c3CNCC5)C4)ccnc2c1 ; CC(C)S(=O)(=O)NCCc1cc2nccc(-c3cc(Cl)cc4c3OC(C(=O)N3CCNCC3)C4)c2s1]

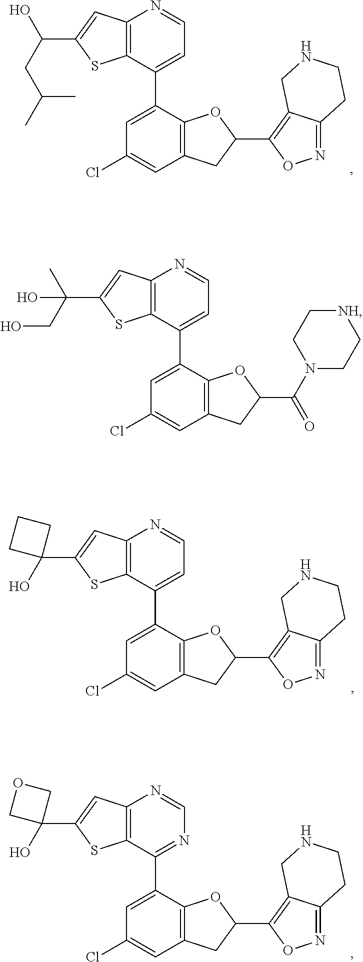 [SMILES: CC(C)CC(O)c1cc2nccc(-c3cc(Cl)cc4c3OC(c3onc5c3CNCC5)C4)c2s1 ; CC(O)(CO)c1cc2nccc(-c3cc(Cl)cc4c3OC(C(=O)N3CCNCC3)C4)c2s1 ; OC1(c2cc3nccc(-c4cc(Cl)cc5c4OC(c4onc6c4CNCC6)C5)c3s2)CCC1 ; OC1(c2cc3ncnc(-c4cc(Cl)cc5c4OC(c4onc6c4CNCC6)C5)c3s2)COC1]

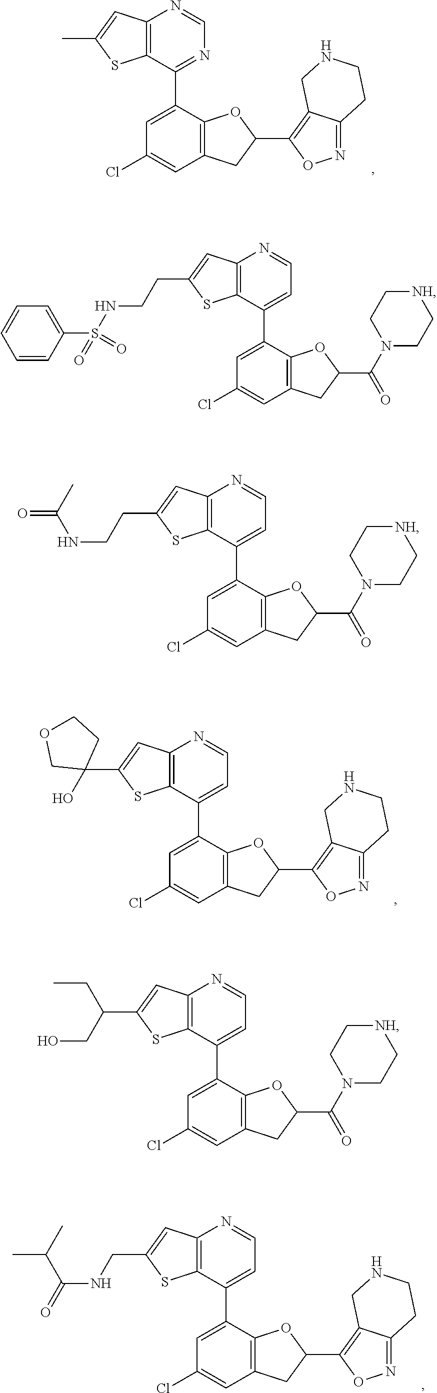 [SMILES: Cc1cc2ncnc(-c3cc(Cl)cc4c3OC(c3onc5c3CNCC5)C4)c2s1 ; O=C(C1Cc2cc(Cl)cc(-c3ccnc4cc(CCNS(=O)(=O)c5ccccc5)sc34)c2O1)N1CCNCC1 ; CC(=O)NCCc1cc2nccc(-c3cc(Cl)cc4c3OC(C(=O)N3CCNCC3)C4)c2s1 ; OC1(c2cc3nccc(-c4cc(Cl)cc5c4OC(c4onc6c4CNCC6)C5)c3s2)CCOC1 ; CCC(CO)c1cc2nccc(-c3cc(Cl)cc4c3OC(C(=O)N3CCNCC3)C4)c2s1 ; CC(C)C(=O)NCc1cc2nccc(-c3cc(Cl)cc4c3OC(c3onc5c3CNCC5)C4)c2s1]

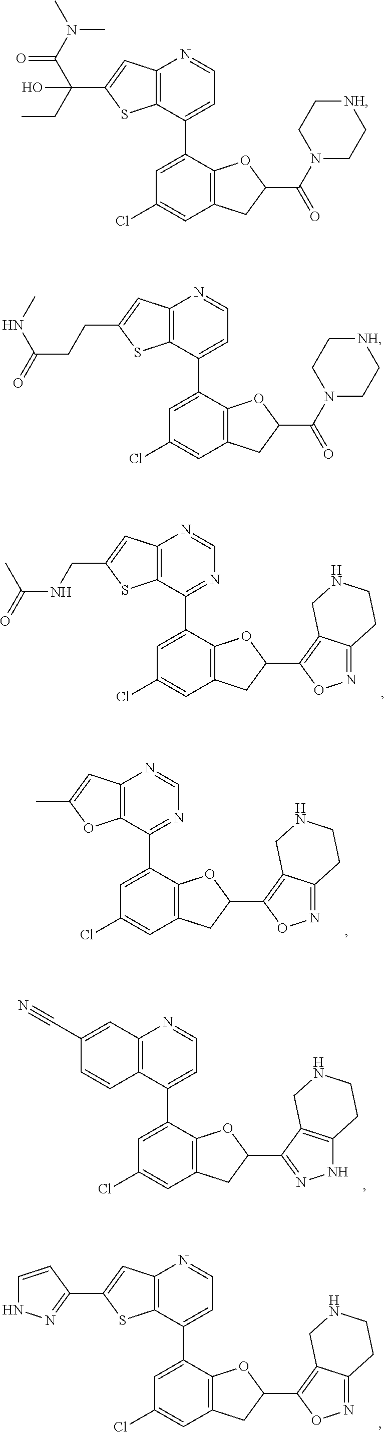 [SMILES: CCC(O)(C(=O)N(C)C)c1cc2nccc(-c3cc(Cl)cc4c3OC(C(=O)N3CCNCC3)C4)c2s1 ; CNC(=O)CCc1cc2nccc(-c3cc(Cl)cc4c3OC(C(=O)N3CCNCC3)C4)c2s1 ; CC(=O)NCc1cc2ncnc(-c3cc(Cl)cc4c3OC(c3onc5c3CNCC5)C4)c2s1 ; Cc1cc2ncnc(-c3cc(Cl)cc4c3OC(c3onc5c3CNCC5)C4)c2o1 ; N#Cc1ccc2c(-c3cc(Cl)cc4c3OC(c3n[nH]c5c3CNCC5)C4)ccnc2c1 ; Clc1cc2c(c(-c3ccnc4cc(-c5cc[nH]n5)sc34)c1)OC(c1onc3c1CNCC3)C2]

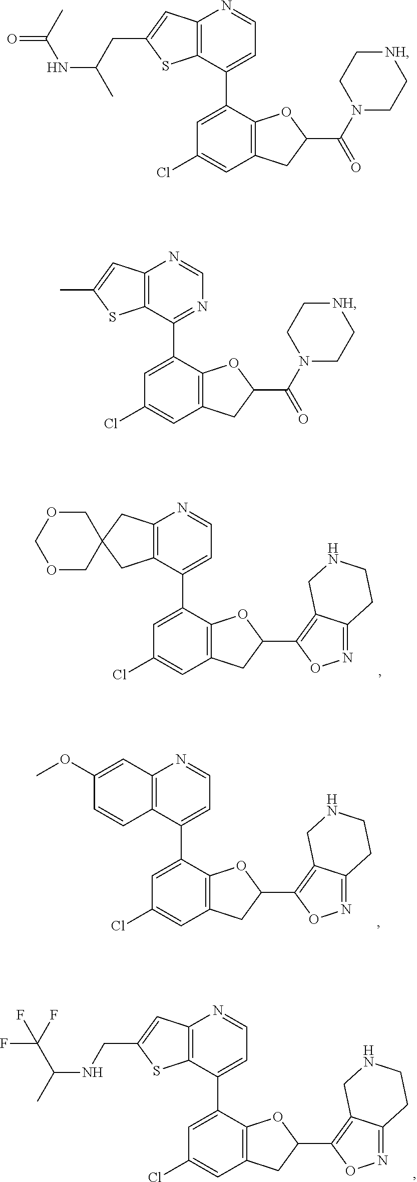 [SMILES: CC(=O)NC(C)Cc1cc2nccc(-c3cc(Cl)cc4c3OC(C(=O)N3CCNCC3)C4)c2s1 ; Cc1cc2ncnc(-c3cc(Cl)cc4c3OC(C(=O)N3CCNCC3)C4)c2s1 ; Clc1cc2c(c(-c3ccnc4c3CC3(COCOC3)C4)c1)OC(c1onc3c1CNCC3)C2 ; COc1ccc2c(-c3cc(Cl)cc4c3OC(c3onc5c3CNCC5)C4)ccnc2c1 ; CC(NCc1cc2nccc(-c3cc(Cl)cc4c3OC(c3onc5c3CNCC5)C4)c2s1)C(F)(F)F]

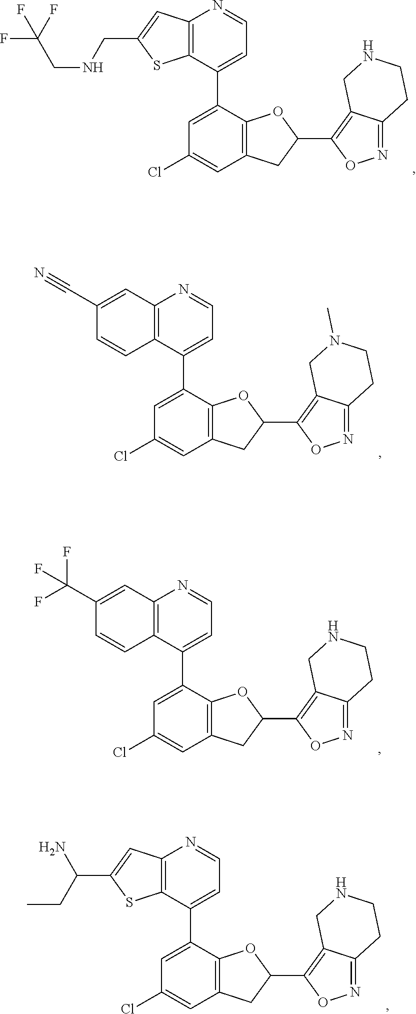 [SMILES: FC(F)(F)CNCc1cc2nccc(-c3cc(Cl)cc4c3OC(c3onc5c3CNCC5)C4)c2s1 ; CN1CCc2noc(C3Cc4cc(Cl)cc(-c5ccnc6cc(C#N)ccc56)c4O3)c2C1 ; FC(F)(F)c1ccc2c(-c3cc(Cl)cc4c3OC(c3onc5c3CNCC5)C4)ccnc2c1 ; CCC(N)c1cc2nccc(-c3cc(Cl)cc4c3OC(c3onc5c3CNCC5)C4)c2s1]

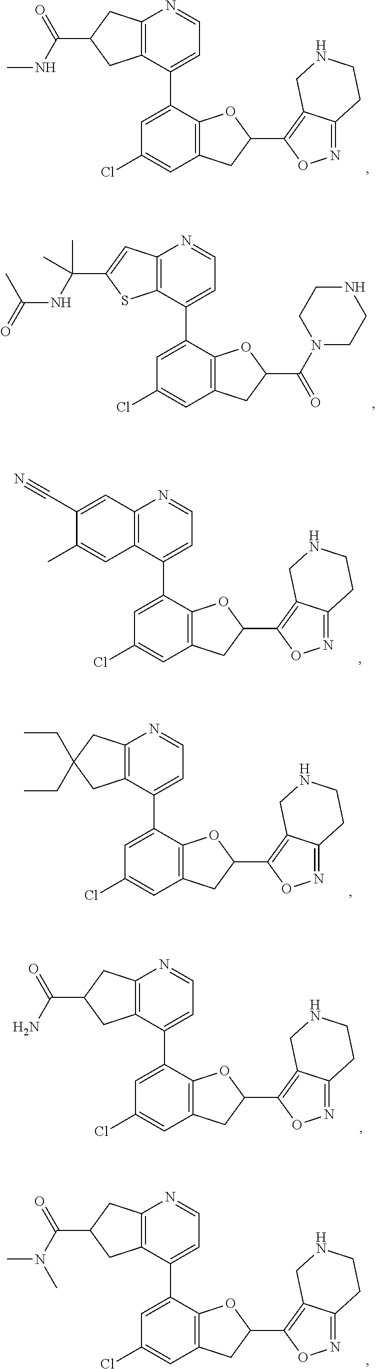 [SMILES: CNC(=O)C1Cc2nccc(-c3cc(Cl)cc4c3OC(c3onc5c3CNCC5)C4)c2C1 ; CC(=O)NC(C)(C)c1cc2nccc(-c3cc(Cl)cc4c3OC(C(=O)N3CCNCC3)C4)c2s1 ; Cc1cc2c(-c3cc(Cl)cc4c3OC(c3onc5c3CNCC5)C4)ccnc2cc1C#N ; CCC1(CC)Cc2nccc(-c3cc(Cl)cc4c3OC(c3onc5c3CNCC5)C4)c2C1 ; NC(=O)C1Cc2nccc(-c3cc(Cl)cc4c3OC(c3onc5c3CNCC5)C4)c2C1 ; CN(C)C(=O)C1Cc2nccc(-c3cc(Cl)cc4c3OC(c3onc5c3CNCC5)C4)c2C1]

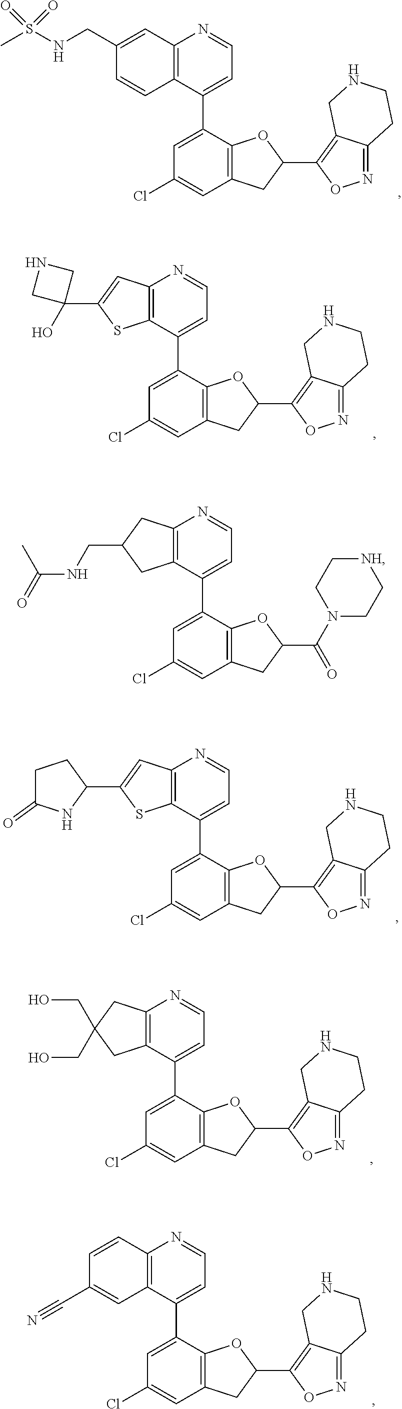 [SMILES: CS(=O)(=O)NCc1ccc2c(-c3cc(Cl)cc4c3OC(c3onc5c3CNCC5)C4)ccnc2c1 ; OC1(c2cc3nccc(-c4cc(Cl)cc5c4OC(c4onc6c4CNCC6)C5)c3s2)CNC1 ; CC(=O)NCC1Cc2nccc(-c3cc(Cl)cc4c3OC(C(=O)N3CCNCC3)C4)c2C1 ; O=C1CCC(c2cc3nccc(-c4cc(Cl)cc5c4OC(c4onc6c4CNCC6)C5)c3s2)N1 ; OCC1(CO)Cc2nccc(-c3cc(Cl)cc4c3OC(c3onc5c3CNCC5)C4)c2C1 ; N#Cc1ccc2nccc(-c3cc(Cl)cc4c3OC(c3onc5c3CNCC5)C4)c2c1]

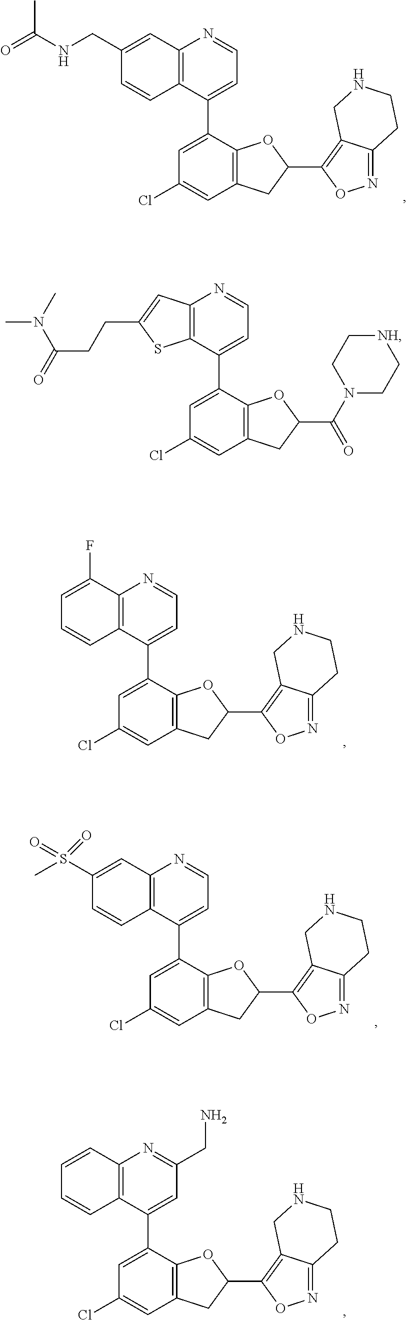 [SMILES: CC(=O)NCc1ccc2c(-c3cc(Cl)cc4c3OC(c3onc5c3CNCC5)C4)ccnc2c1 ; CN(C)C(=O)CCc1cc2nccc(-c3cc(Cl)cc4c3OC(C(=O)N3CCNCC3)C4)c2s1 ; Fc1cccc2c(-c3cc(Cl)cc4c3OC(c3onc5c3CNCC5)C4)ccnc12 ; CS(=O)(=O)c1ccc2c(-c3cc(Cl)cc4c3OC(c3onc5c3CNCC5)C4)ccnc2c1 ; NCc1cc(-c2cc(Cl)cc3c2OC(c2onc4c2CNCC4)C3)c2ccccc2n1]

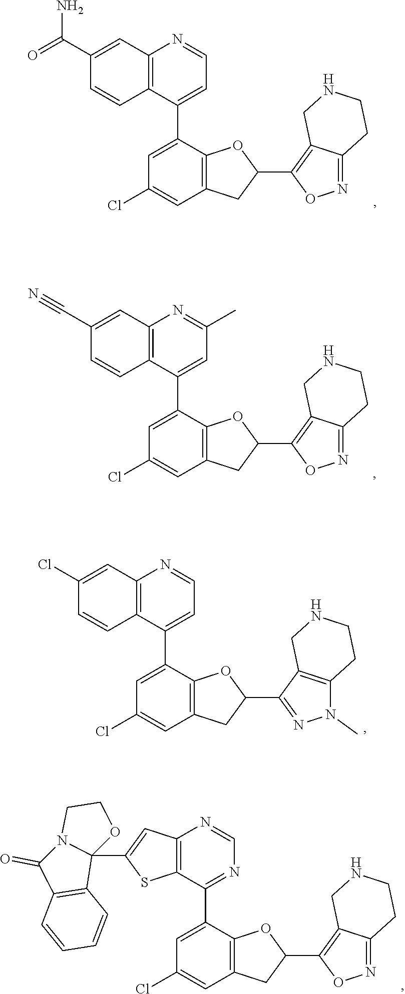 [SMILES: NC(=O)c1ccc2c(-c3cc(Cl)cc4c3OC(c3onc5c3CNCC5)C4)ccnc2c1 ; Cc1cc(-c2cc(Cl)cc3c2OC(c2onc4c2CNCC4)C3)c2ccc(C#N)cc2n1 ; Cn1nc(C2Cc3cc(Cl)cc(-c4ccnc5cc(Cl)ccc45)c3O2)c2c1CCNC2 ; O=C1c2ccccc2C2(c3cc4ncnc(-c5cc(Cl)cc6c5OC(c5onc7c5CNCC7)C6)c4s3)OCCN12]

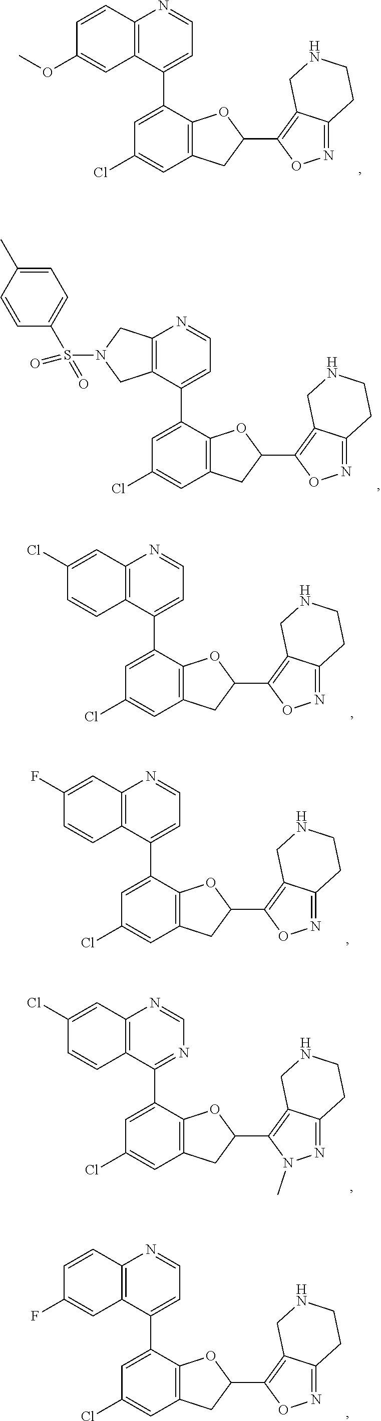 [SMILES: COc1ccc2nccc(-c3cc(Cl)cc4c3OC(c3onc5c3CNCC5)C4)c2c1 ; Cc1ccc(S(=O)(=O)N2Cc3nccc(-c4cc(Cl)cc5c4OC(c4onc6c4CNCC6)C5)c3C2)cc1 ; Clc1cc2c(c(-c3ccnc4cc(Cl)ccc34)c1)OC(c1onc3c1CNCC3)C2 ; Fc1ccc2c(-c3cc(Cl)cc4c3OC(c3onc5c3CNCC5)C4)ccnc2c1 ; Cn1nc2c(c1C1Cc3cc(Cl)cc(-c4ncnc5cc(Cl)ccc45)c3O1)CNCC2 ; Fc1ccc2nccc(-c3cc(Cl)cc4c3OC(c3onc5c3CNCC5)C4)c2c1]

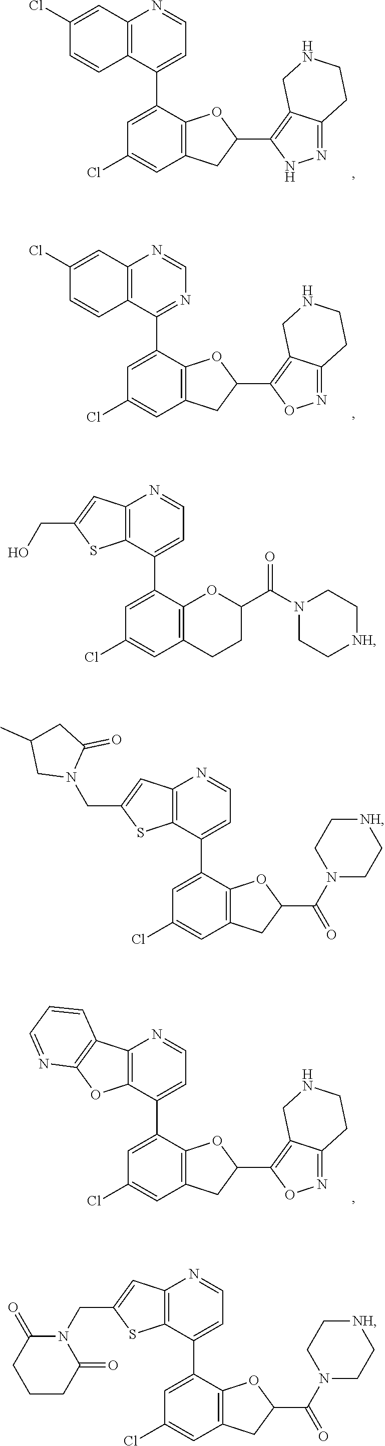 [SMILES: Clc1cc2c(c(-c3ccnc4cc(Cl)ccc34)c1)OC(c1[nH]nc3c1CNCC3)C2 ; Clc1cc2c(c(-c3ncnc4cc(Cl)ccc34)c1)OC(c1onc3c1CNCC3)C2 ; O=C(C1CCc2cc(Cl)cc(-c3ccnc4cc(CO)sc34)c2O1)N1CCNCC1 ; CC1CC(=O)N(Cc2cc3nccc(-c4cc(Cl)cc5c4OC(C(=O)N4CCNCC4)C5)c3s2)C1 ; Clc1cc2c(c(-c3ccnc4c3oc3ncccc34)c1)OC(c1onc3c1CNCC3)C2 ; O=C(C1Cc2cc(Cl)cc(-c3ccnc4cc(CN5C(=O)CCCC5=O)sc34)c2O1)N1CCNCC1]

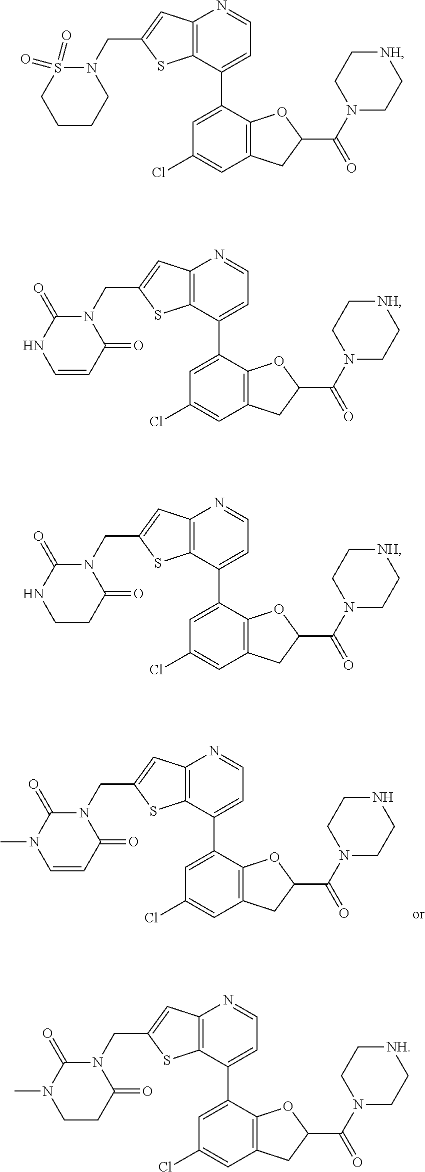 [SMILES: O=C(C1Cc2cc(Cl)cc(-c3ccnc4cc(CN5CCCCS5(=O)=O)sc34)c2O1)N1CCNCC1 ; O=C(C1Cc2cc(Cl)cc(-c3ccnc4cc(Cn5c(=O)cc[nH]c5=O)sc34)c2O1)N1CCNCC1 ; O=C(C1Cc2cc(Cl)cc(-c3ccnc4cc(CN5C(=O)CCNC5=O)sc34)c2O1)N1CCNCC1 ; Cn1ccc(=O)n(Cc2cc3nccc(-c4cc(Cl)cc5c4OC(C(=O)N4CCNCC4)C5)c3s2)c1=O ; CN1CCC(=O)N(Cc2cc3nccc(-c4cc(Cl)cc5c4OC(C(=O)N4CCNCC4)C5)c3s2)C1=O]

62. A pharmaceutical composition, comprising the compound of claim 1 and a pharmaceutically acceptable excipient.

63. A method of inhibiting ubiquitin-specific-processing protease 7 (USP7), comprising contacting USP7 with a compound of claim 1.

64. A method of treating a USP7-mediated disease or disorder, comprising administering to a patient in need thereof a therapeutically effective amount of a compound of claim 1.

65. The method of claim 64, wherein the USP7-mediated disease or disorder is an immune disorder, a cardiovascular disease, a viral infection, inflammation, a metabolism/endocrine disorder or a neurological disorder.

66. The method of claim 64, wherein the USP7-mediated disease or disorder is cancer.

67. The method of claim 66, wherein the cancer is breast cancer, ovarian cancer, cervical cancer, prostate cancer, cancer of the testes, genitourinary tract cancer, cancer of the esophagus, cancer of the larynx, glioblastoma, neuroblastoma, stomach cancer, skin cancer, keratoacanthoma, lung cancer, epidermoid carcinoma, large cell carcinoma, non-small cell lung carcinoma (NSCLC), small cell carcinoma, lung adenocarcinoma, bone cancer, colon cancer, adenoma, pancreatic cancer, adenocarcinoma, thyroid cancer, follicular carcinoma, undifferentiated carcinoma, papillary carcinoma, seminoma, melanoma, sarcoma, bladder carcinoma, liver carcinoma and biliary passages, kidney carcinoma, lymphoma, cancer of the pharynx, cancer of the lip, cancer of the tongue, cancer of the mouth, cancer of the small intestine, colorectal cancer, cancer of the large intestine, rectal cancer, brain cancer Hodgkin's, leukemia, cancer of the bronchus, cancer of the thyroid, liver cancer, intrahepatic bile duct cancer, gastric cancer, glioma/glioblastoma, endometrial cancer, melanoma, kidney cancer, renal cancer, pelvic cancer, urinary bladder cancer, multiple myeloma, acute myelogenous leukemia, chronic myelogenous leukemia, lymphocytic leukemia, chronic lymphoid leukemia (CLL), myeloid leukemia, cancer of the oral cavity and pharynx, non-Hodgkin lymphoma, melanoma or villous colon adenoma.

68. The method of claim 66, further comprising administering to the patient a chemotherapeutic agent in combination with the compound of claim 1.
